(19) United States
(12) Patent Application Publication
(10) Pub. No.: US 2025/0235531 A1
(43) Pub. Date: Jul. 24, 2025

(54) MUCOSAL ADMINISTRATION METHODS AND FORMULATIONS

(71) Applicant: MODERNATX, INC., Cambridge, MA (US)

(72) Inventors: Darin Edwards, Cambridge, MA (US); Gaby Baldeon Vaca, Cambridge, MA (US); Kapil Bahl, Medford, MA (US); Ana Cadete Pires

(73) Assignee: ModernaTX, Inc., Cambridge, MA (US)

(21) Appl. No.: 18/836,530

(22) PCT Filed: Feb. 9, 2023
(86) PCT No.: PCT/US2023/062315

§ 371 Date: Aug. 7, 2024

Related U.S. Application Data
(60) Provisional application No. 63/437,070, filed on Jan. 4, 2023.
(60) Provisional application No. 63/408,799, filed on Sep. 21, 2022.
(60) Provisional application No. 63/308,409, filed on Feb. 9, 2022.

(51) Int. Cl. A61K 39/385; A61K 39/00

(52) U.S. Cl. CPC A61K 39/385; A61K 2039/53; A61K 2039/541; A61K 2039/542; A61K 2039/543; A61K 2039/544; A61K 2039/6018

(57) ABSTRACT

The present disclosure provides compositions and methods for the preparation, manufacture, and therapeutic use of lipid nanoparticles comprising nucleic acid vaccines, e.g., mRNA vaccines, for delivery to mucosal surfaces.

### RELATED APPLICATIONS

This application claims the benefit under 35 U.S.C. § 119(e) of U.S. provisional application No. 63/308,409, filed Feb. 9, 2022, U.S. provisional application No. 63/408,799, filed Sep. 21, 2022, and U.S. provisional application No. 63/437,070, filed Jan. 4, 2023, each of which is incorporated by reference herein in its entirety.

### REFERENCE TO AN ELECTRONIC SEQUENCE LISTING

The contents of the electronic sequence listing (M137870218W000-SEQ-JXV.xml; Size: 59,862 bytes; and Date of Creation: Feb. 7, 2023) is herein incorporated by reference in its entirety.

### BACKGROUND

The mucosa is a mucous membrane that lines various cavities in the body, covering the surface of internal organs. It comprises one or more layers of epithelial cells overlying a layer of loose connective tissue. The function of the mucosa is to prevent pathogens and harmful foreign substances from entering the body and to prevent bodily tissues from becoming dehydrated.

One example of a mucosal cells is the respiratory epithelial cell. Respiratory epithelial cells line the respiratory tract. The primary functions of the respiratory epithelial cells are to moisten the respiratory tract, protect the airway tract from potential pathogens, infections and tissue injury, and/or facilitate gas exchange. Delivery of payloads to respiratory epithelial cells can be used to induce immunity to antigens of interest (e.g., vaccination and therapeutic delivery) or to treat other disorders that would benefit from therapeutic delivery of nucleic acid molecules or other payload molecules to airway epithelial cells.

### SUMMARY

The present disclosure provides lipid nanoparticles (LNPs) for delivery of polynucleotide or polypeptide payloads, e.g., nucleic acid molecules, mRNA vaccines and nucleic acid therapeutics, to the mucosa (e.g., airway epithelial cells) for the prevention and/or treatment of diseases, including respiratory diseases. In one embodiment, the subject LNPs can be used to administer nucleic acid vaccines and/or therapeutics. The instant disclosure provides LNPs which have improved properties when administered to cells, e.g., in vitro and in vivo, for example, improved delivery of payloads to mucosal cells as measured, e.g., by cellular accumulation of LNP, expression of a desired protein, and/or mRNA expression. For example, intranasal delivery of mRNA vaccines was found to result in meaningful immunogenic responses, as measured by, e.g., neutralization titers and binding assays.

The disclosure, in some aspects, provides a method for inducing a mucosal immune response, comprising administering to a mucosal surface of a subject a composition comprising an mRNA encoding an antigen and a nanoparticle, wherein the nanoparticle comprises a lipid nanoparticle core comprising an ionizable lipid, a phospholipid, a structural lipid, and a PEG-lipid, and a cationic agent dispersed primarily on the outer surface of the core in an effective amount to induce a mucosal immune response.

In some embodiments, the mRNA is encapsulated within the core. In some embodiments, the nanoparticle has a greater than neutral zeta potential at physiological pH. In some embodiments, a weight ratio of the cationic agent to nucleic acid vaccine is about 1:1 to about 4:1, about 1.25:1 to about 3.75:1, about 1.25:1, about 2.5:1, or about 3.75:1.

In some embodiments, the antigen is an infectious disease antigen.

In some embodiments, the mucosal surface comprises a cell population selected from respiratory mucosal cells, oral mucosal cells, intestinal mucosal cells, vaginal mucosal cells, rectal mucosal cells, and buccal mucosal cells.

The disclosure, in some aspects, provides a method for expressing a protein in mucosal tissue, comprising administering to a mucosal surface of a subject a composition comprising an mRNA encoding an protein and a nanoparticle, wherein the nanoparticle comprises a lipid nanoparticle core comprising an ionizable lipid, a phospholipid, a structural lipid, and a PEG-lipid, and a cationic agent dispersed primarily on the outer surface of the core in an effective amount to induce expression of the protein in a mucosal tissue.

In some embodiments, the mRNA encodes a therapeutic protein. In some embodiments, the mRNA is encapsulated within the core. In some embodiments, the nanoparticle has a greater than neutral zeta potential at physiological pH. In some embodiments, a weight ratio of the cationic agent to nucleic acid vaccine is about 1:1 to about 4:1, about 1.25:1 to about 3.75:1, about 1.25:1, about 2.5:1, or about 3.75:1.

In some embodiments, the mucosal surface comprises a cell population selected from respiratory mucosal cells, oral mucosal cells, intestinal mucosal cells, vaginal mucosal cells, rectal mucosal cells, and buccal mucosal cells.

The disclosure, in some embodiments, provides a composition comprising an mRNA vaccine, comprising an mRNA comprising an open reading frame encoding an antigen and a nanoparticle, wherein the nanoparticle comprises a lipid nanoparticle core comprising an ionizable lipid, a phospholipid, a structural lipid, a PEG-lipid, and the mRNA, and a cationic agent dispersed primarily on the outer surface of the core.

In some embodiments, the antigen is an infectious disease antigen. In some embodiments, the infectious disease antigen is a viral antigen.

The disclosure, in some aspects, provides a composition comprising an mRNA therapeutic, comprising an mRNA comprising an open reading frame encoding a therapeutic protein, wherein the therapeutic protein is not a lung protein and a nanoparticle, wherein the nanoparticle comprises a lipid nanoparticle core comprising the mRNA and a cationic agent dispersed primarily on the outer surface of the core.

In some embodiments, the mRNA is encapsulated within the core. In some embodiments, the nanoparticle has a greater than neutral zeta potential at physiological pH. In some embodiments, a weight ratio of the cationic agent to nucleic acid vaccine is about 1:1 to about 4:1, about 1.25:1 to about 3.75:1, about 1.25:1, about 2.5:1, or about 3.75:1. In some embodiments, the nanoparticle has a zeta potential of about 5 mV to about 20 mV, about 5 mV to about 20 mV, about 5 mV to about 15 mV, or about 5 mV to about 10 mV.

In some embodiments, greater than about 80%, greater than 90%, greater than 95%, or greater than 95% of the cationic agent is on the surface on the nanoparticle. In some embodiments, at least about 50%, at least about 75%, at least about 90%, or at least about 95% of the mRNA is encapsulated within the core.

In some embodiments, a general polarization of laurdan (GPL) of the nanoparticle is greater than or equal to about 0.6. In some embodiments, the nanoparticle has a d-spacing of greater than about 6 nm or greater than about 7 nm. In some embodiments, at least 50%, at least 75%, at least 90%, or at least 95% of the nanoparticles have a surface fluidity value of greater than a threshold polarization level. In some embodiments, about 10% or greater, about 15% or greater, or about 20% or greater of cell population has accumulated the nanoparticle when the nanoparticle is contacted with a population of mucosal cells.

In some embodiments, the cationic agent has a solubility of greater than about 1 mg/mL, greater than about 5 mg/mL, greater than about 10 mg/mL, or greater than about 20 mg/mL in alcohol.

In some embodiments, the cationic agent is a cationic lipid and the cationic lipid is a water-soluble amphiphilic molecule. In some embodiments, the amphiphilic molecule comprises a lipid moiety and a hydrophilic moiety. In some embodiments, the cationic agent is a cationic lipid and the cationic lipid comprises a structural lipid, fatty acid, or hydrocarbyl group. In some embodiments, the cationic agent is a cationic lipid and the cationic lipid is a sterol amine comprising a hydrophobic moiety and a hydrophilic moiety.

In some embodiments, the hydrophilic moiety comprises an amine group comprising one to four primary, secondary, or tertiary amines or mixtures thereof. In some embodiments, the amine group comprises one or two terminal primary amines. In some embodiments, the amine group comprises one or two terminal primary amines and one internal secondary amine. In some embodiments, the amine group comprises one or two tertiary amines. In some embodiments, the amine group has a pKa value of greater than about 8. In some embodiments, the amine group has a pKa value of greater than about 9.

In some embodiments, the sterol amine is a compound of Formula (A1): A-L-B (A1) or a salt thereof, wherein: A is an amine group, L is an optional linker, and B is a sterol.

In some embodiments, the sterol amine has Formula A2a:

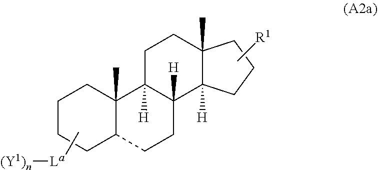

or a salt thereof, wherein:
[symbol] is a single or double bond
R¹ is C₁₋₁₄ alkyl or C₁₋₁₄ alkenyl;
L^{a} is absent, —O—, —S—S—, —OC(═O), —C(═O)N—, —OC(═O)N—, CH₂—NH—C(O)—, —C(═O)O—, —OC(═O)—CH₂—CH₂—C(═O)N—, —S—S—CH₂—, —SS—CH₂—CH₂—C(═O)N—, or a group of formula (a):

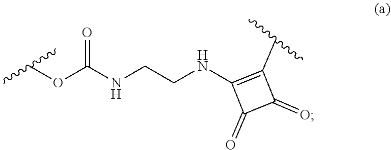 [SMILES: *OC(=O)NCCNc1c(*)c(=O)c1=O]

Y¹ is C₁₋₁₀ alkyl, 3 to 8-membered heterocycloalkyl, 5 to 6-membered heteroaryl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) are each optionally substituted with 1, 2, 3, or 4 substituents independently selected from C₁₋₆ alkyl, halo, —OH, —O(C₁₋₆ alkyl), —C₁₋₆ alkyl-OH, —NH₂, —NH(C₁₋₆ alkyl), —N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH-(3 to 8-membered heterocycloalkyl), and —NH(5 to 6-membered heteroaryl); and
n is 1 or 2, and
   optionally:
wherein [symbol] is a double bond,
wherein [symbol] is a single bond,
wherein L^{a} is —OC(═O)—, —OC(═O)N—, or —OC(═O)—CH₂—CH₂—C(═O)N—,
wherein n is 1,
wherein n is 2,
wherein R¹ is C₁₋₁₄ alkyl,
wherein R¹ is C₁₋₁₄ alkenyl
wherein R¹ is

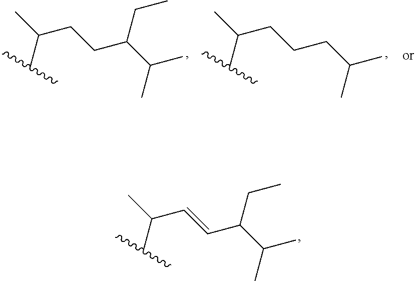

and/or
wherein Y¹ is C₁₋₁₀ alkyl, 3 to 8-membered heterocycloalkyl, —C₁₋₆ alkyl-(3 to 8-membered heterocycloalkyl), or —C₁₋₆ alkyl-(5 to 6-membered heteroaryl), wherein the C₁₋₁₀ alkyl, 3 to 8-membered heterocycloalkyl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof; and wherein the C₁₋₁₀ alkyl, C₁₋₆ alkyl-(3 to 8-membered heterocycloalkyl), and C₁₋₆ alkyl-(5 to 6-membered heteroaryl) are each optionally substituted with C₁₋₆ alkyl, —OH, —C₁₋₆alkyl-OH, or —NH₂.

In some embodiments, Y¹ is selected from:

(1);

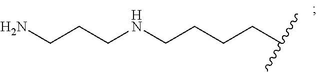 [SMILES: *CCCCNCCCN]

(2)

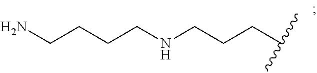 [SMILES: *CCCNCCCCN]

(3)

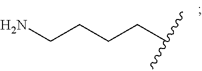 [SMILES: *CCCCN]

(4)

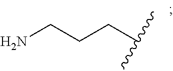 [SMILES: *CCCN]

(5)

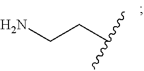 [SMILES: *CCN]

(6)

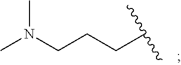 [SMILES: *CCCN(C)C]

(7)

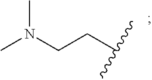 [SMILES: *CCN(C)C]

(8)

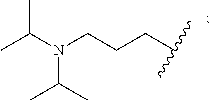 [SMILES: *CCCN(C(C)C)C(C)C]

(9)

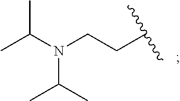 [SMILES: *CCN(C(C)C)C(C)C]

(10)

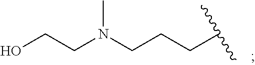 [SMILES: *CCCN(C)CCO]

(11)

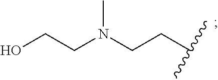 [SMILES: *CCN(C)CCO]

(12)

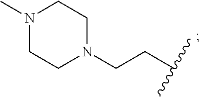 [SMILES: *CCN1CCN(C)CC1]

(13)

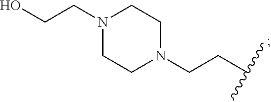 [SMILES: *CCN1CCN(CCO)CC1]

(14)

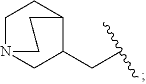 [SMILES: *CC1CN2CCC1CC2]

(15)

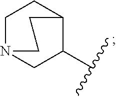 [SMILES: *C1CN2CCC1CC2]

(16)

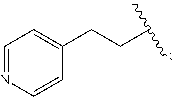 [SMILES: *CCc1ccncc1]

(17)

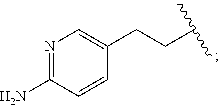 [SMILES: *CCc1ccc(N)nc1]

(18)

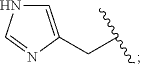 [SMILES: *Cc1c[nH]cn1]

(19)

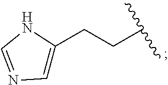 [SMILES: *CCc1cnc[nH]1]

(20)

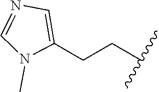 [SMILES: *CCc1cncn1C]

(21)

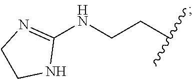 [SMILES: *CCNC1=NCCN1]

(22)

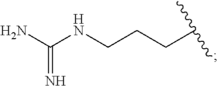 [SMILES: *CCCNC(=N)N]

(23)

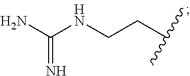 [SMILES: *CCNC(=N)N]

(28) —N(CH₃)₂; (29)

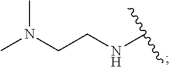 [SMILES: *NCCN(C)C]

(30)

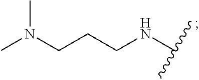 [SMILES: *NCCCN(C)C]

(31)

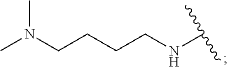 [SMILES: *NCCCCN(C)C]

and (32)

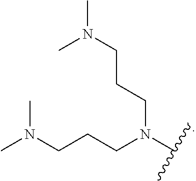 [SMILES: *N(CCCN(C)C)CCCN(C)C]

In some embodiments, the sterol amine has Formula A4:

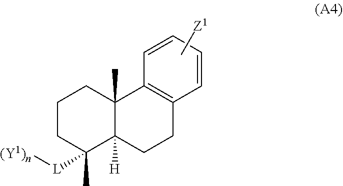

or a salt thereof, wherein:
Z¹ is —OH or C₃₋₆ alkyl;
L is absent, —O—, —S—S—, —OC(═O)—, —C(═O)N—, —OC(═O)N—, —CH₂—NH—C(═O)—, —C(═O)O—, —OC(═O)—CH₂—CH₂—C(═O)N—, —S—S—CH₂—, or —SS—CH₂—CH₂—C(O)N—;
Y¹ is C₁₋₁₀ alkyl, 3 to 8-membered heterocycloalkyl, 5 to 6-membered heteroaryl, —C₁₋₆ alkyl-(3 to 8 membered heterocycloalkyl), or —C₁₋₆ alkyl-(5 to 6 membered heteroaryl),
wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, 3 to 8-membered heterocycloalkyl, 5 to 6 membered heteroaryl, —C₁₋₆ alkyl-(3 to 8-membered heterocycloalkyl), and —C₁₋₆ alkyl-(5 to 6-membered heteroaryl) are each optionally substituted with 1, 2, 3, or 4 substituents selected from C₁₋₆ alkyl, halo, —OH, —O(C₁₋₆ alkyl), —C₁₋₆ alkyl-OH, —NH₂, —NH(C₁₋₆ alkyl), —N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH(3 to 8-membered heterocycloalkyl), and —NH(5 to 6-membered heteroaryl); and
n is 1 or 2, and
   optionally:
wherein Z¹ is —OH,
wherein Z¹ is C₃₋₆ alkyl,
wherein L is —C(═O)N—, —CH₂—NH—C(═O)—, or —C(═O)O—,
wherein Y¹ is C₁₋₁₀ alkyl comprising one to five primary, secondary, or tertiary amines or combination thereof,
wherein Y¹ is

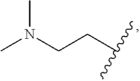

wherein n is 1, and/or
wherein n is 2.

In some embodiments, the sterol amine is selected from: SA3, SA10, SA18, SA24, SA58, SA78, SA121, SA137, SA138, SA158, and SA183. In some embodiments, the cationic agent is a non-lipid cationic agent. In some embodiments, the non-lipid cationic agent is benzalkonium chloride, cetylpyridium chloride, L-lysine monohydrate, or tromethamine. In some embodiments, the cationic agent is a modified arginine.

In some embodiments, the nanoparticle comprises about 30 mol % to about 60 mol % or about 40 mol % to about 50 mol % of ionizable lipid.

In some embodiments, the ionizable lipid is a compound of Formula (I):

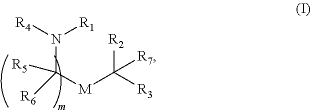

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR⁹)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR₉)N(R)₂, —C(═NR⁹)N(R)₂, —C(═NR⁹)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, —CN, —NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₃ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, the nanoparticle comprises about 5 mol % to about 15 mol %, about 8 mol % to about 13 mol %, or about 10 mol % to about 12 mol % of phospholipid. In some embodiments, the phospholipid is 1,2-distearoyl-sn-glycero-3-phosphocholine (DSPC).

In some embodiments, the nanoparticle comprises about 20 mol % to about 60 mol %, about 30 mol % to about 50 mol %, about 35 mol %, or about 40 mol % structural lipid.

In some embodiments, the mRNA is in a nebulizer or inhaler or droplet.

In some embodiments, the mRNA encoding a therapeutic protein does not comprise a cystic fibrosis transmembrane conductance regulator (CFTR) protein.

### BRIEF DESCRIPTION OF THE DRAWINGS

FIG. 1 is a diagram of exemplary first generation post-hoc loading (PHL) process for preparing LNP.

FIG. 2 is a diagram of exemplary second generation PHL process (generic) for preparing LNP.

FIG. 3 is a diagram of exemplary second generation PHL process (specific) for preparing LNP.

FIG. 4 is a diagram of exemplary process of preparing an empty lipid nanoparticle prototype (“Neutral assembly”), where the empty LNP is mixed at pH 8.0 and the final formulation is pH 5.0.

FIG. 5 is a diagram of exemplary process of preparing an LNP with a sterol amine.

FIGS. 6A-6D are graphs showing the expression of luciferase in mice 6 hours (FIGS. 6A and 6B) and 24 hours (FIGS. 6C and 6D) after intranasal administration of mRNA encoding luciferase formulated in lipid nanoparticles. The results were quantified using whole body IVIS imaging, focusing on the nasal cavity (FIGS. 6A and 6C) and lungs (FIGS. 6B and 6D).

FIGS. 7A-7B are graphs showing percentage of V5-positive cells relative to the total number of cells (FIG. 7A) and number of V5-positive cells (FIG. 7B) in mice six and 24 hours after intranasal administration of mRNA encoding luciferase formulated in lipid nanoparticles. Cells were counted at three different levels of the nasal cavity (1 represents the region most cranial and 3 represents the region most caudal).

FIGS. 8A-8D are graphs showing antigen-specific binding titers in hamster sera after intranasal administration of an mRNA vaccine comprising an open reading frame (ORF) encoding Antigen 1 (AG1) in nanoparticle (FIG. 8A), the neutralizing titers in hamster sera after intranasal administration of an mRNA vaccine comprising an ORF encoding AG1 in nanoparticle (FIG. 8B), the percent change in body weight in hamsters following administration of two doses of an mRNA vaccine comprising an ORF encoding AG1 in nanoparticle and challenge with a virus comprising AG1 (FIG. 8C), and viral load in different compartments 3 days after challenge (FIG. 8D). In FIGS. 8A-8D, “Compound SA3” represents an LNP comprising SA3 and compound 18, and “Compound SA23” represents an LNP comprising SA23 and compound 18.

FIG. 9 is a series of graphs shown the IgG binding titers resulting following intranasal administration of an mRNA vaccine comprising an ORF encoding Antigen2 (AG2) formatted in respiratory LNPs. The results following administration of low doses (5 μg) or high doses (20 μg) are shown in the top and bottom panels, respectively. In FIG. 9, “Compound SA3” represents an LNP comprising SA3 and compound 18, and “Compound SA23” represents an LNP comprising SA23 and compound 18.

FIG. 10 is a series of graphs showing the IgA binding titers resulting following intranasal administration of an mRNA vaccine comprising an ORF encoding AG2 formatted in respiratory LNPs. The results following administration of low doses (5 μg) or high doses (20 μg) are shown in the top and bottom panels, respectively. In FIG. 10, “Compound SA3” represents an LNP comprising SA3 and compound 18, and “Compound SA23” represents an LNP comprising SA23 and compound 18.

FIG. 11 is a series of graphs showing the results of a B-cell ELISpot assay following administration of two high doses (20 μg) of an mRNA vaccine comprising an ORF encoding AG2 formatted in respiratory LNPs administered intranasally in mice. In FIG. 11, “Compound SA3” represents an LNP comprising SA3 and compound 18, and “Compound SA10” represents an LNP comprising SA10 and compound 18.

FIG. 12 is a series of graphs showing the results of a B-cell ELISspot assay following administration of two low doses (5 μg) of an mRNA vaccine comprising an ORF encoding AG2 formatted in respiratory LNPs administered intranasally in mice. In FIG. 12, “Compound SA3” represents an LNP comprising SA3 and compound 18, and “Compound SA10” represents an LNP comprising SA10 and compound 18.

FIG. 13 is two graphs showing neutralization results following administration of two high doses (20 μg, right) or two low doses (5 μg, left) of an mRNA vaccine comprising an ORF encoding AG2 formatted in respiratory LNPs administered intranasally in mice. In FIG. 13, “Compound SA3” represents an LNP comprising SA3 and compound 18, and “Compound SA10” represents an LNP comprising SA10 and compound 18.

FIG. 14 is a series of graphs showing the percent of CD4+ cells (top) and percent of CD8+ cells (bottom) measured after intranasal administration of an mRNA vaccine comprising an ORF encoding AG1 formatted in respiratory LNPs in mice. In FIG. 14, “Compound SA3” represents an LNP comprising SA3 and compound 18, and “Compound SA10” represents an LNP comprising SA10 and compound 18.

FIGS. 15A-15D are graphs showing the protein levels of COV2-2072 antibodies (in ng/mL) detected in sera (FIG. 15A), lung (FIG. 15B), nasal washes (FIG. 15C), and bronchoalveolar lavage fluid (FIG. 15D) in BALB/c mice at hours 0, 24, 48, 72, and 96 post-intranasal administration of a mRNA vaccine encapsulated in different LNP formulations.

FIGS. 16A-16E are graphs showing percentage of each compartment targeted after administration of an mRNA vaccine (10 μL or 25 μL dose) encapsulated in different LNP formulations and administered intravenously (FIG. 16A) or intranasally (FIG. 16B-16E).

FIGS. 17A-17D are graphs showing Luciferase expression measured by bioluminescence imaging in flux (photons per second) on the dorsal side 6 hours (FIG. 17A) and 18 hours (FIG. 17B) after oral administration of a Luciferase mRNA encapsulated in a LNP and on the ventral side 6 hours (FIG. 17C) and 18 hours (FIG. 17D) after intranasal administration of a Luciferase mRNA encapsulated in an LNP.

FIGS. 18A-18D are graphs showing Luciferase expression in the dorsal nose (FIG. 18A), dorsal lung (FIG. 18B), ventral nose (FIG. 18C), and ventral lung (FIG. 18D) measured by bioluminescence imaging in flux (photons per second) at 6 hours and 18 hours after intranasal administration of a Luciferase mRNA encapsulated in an LNP.

FIG. 19 shows an immunization schedule to evaluate the immunogenicity and efficacy of vaccine compositions for HSV-2 administered intramuscularly or intranasally in guinea pigs against a PBS control and a positive control.

FIG. 20 is a schematic illustrating a study design (see Example 28). Intranasal vaccination of an mRNA-based SARS-CoV-2 vaccine was evaluated in Syrian golden hamsters. Hamsters (n=10 per group) were intranasally immunized with 2 doses (Day 0 and Day 21) of vaccines (5 μg or 25 μg) formulated in 2 different LNP compositions or were mock-vaccinated with 2 doses of tris/sucrose buffer administered intranasally; separate groups of animals were intramuscularly immunized with 2 doses of vaccine (0.4 μg or 1 μg). Sera were collected 3 weeks after dose 1 (Day 21) and 3 weeks after dose 2 (Day 41). At Day 42, hamsters were intranasally challenged with SARS-CoV-2 (2019-nCOV/USA-WA1/2020). Post-viral challenge assessments included viral load and histopathology (3 days [Day 45] and 14 days [Day 56] after challenge), immunohistochemistry (3 and 14 days after challenge), as well as body weight (daily after challenge). IM, intramuscular; IN, intranasal; LNP, lipid nanoparticle; mRNA, messenger RNA; PFU, plaque-forming units; SARS-CoV-2, severe acute respiratory syndrome coronavirus 2.

FIGS. 21A-21C show S-specific serum binding IgG antibody (FIG. 21A), S-specific serum binding IgA antibody (FIG. 21B), and serum neutralizing antibody reciprocal endpoint titers (FIG. 21C) (log scale) at 3 weeks after dose 1 (Day 21) or 3 weeks after dose 2 (Day 41) by vaccine group. In each panel, animal-level data are shown as dots (n=9-10 animals per group), with boxes and horizontal bars denoting the IQR and median, respectively, and whiskers representing the maximum and minimum values. Geometric mean titers for each vaccine group are indicated by the plus (+) symbol of each boxplot, with the exact values shown above each vaccine group. Horizontal dotted lines represent the LLOD. *P<0.05, **P<0.01, ***P<0.001, ****P<0.0001. Antibodies were under the limit of detection for all hamsters in the mRNA-LNP1 5 μg group after dose 1, which had a much lower antibody level compared to other groups. IgA, immunoglobulin A; IgG, immunoglobulin G; IM, intramuscular; IN, intranasal; LLOD, lower limit of detection; LNP, lipid nanoparticle; mRNA, messenger RNA; S2-P, S-protein with 2 proline mutations; SD, standard deviation.

FIGS. 22A-22C illustrate viral load and weight loss characteristics after SARS-CoV-2 challenge in vaccinated hamsters. FIG. 22A shows the viral load (PFU per gram of tissue) in lungs and FIG. 22B shows the viral load in nasal turbinates of mock-vaccinated and vaccinated hamsters at 3 days and 14 days after SARS-CoV-2 challenge. Animal-level data are shown as dots (n=5 animals per group), with grey lines representing the geometric mean titer for each group; exact values are shown above each vaccine group. Statistical comparisons were only performed for viral loads at day 3 after challenge, as viral loads at day 14 were zero for all hamsters. *P<0.05, **P<0.01, ***P<0.001, ****P<0.0001. FIG. 22C shows the mean percentage of weight change (error bars represent SEM) over 14 days after SARS-CoV-2 challenge in mock-vaccinated and vaccinated hamsters. IM, intramuscular; IN, intranasal; LNP, lipid nanoparticle; mRNA, messenger RNA; PFU, plaque-forming units; SARS-CoV-2, severe acute respiratory syndrome coronavirus 2; SEM, standard error of the mean.

FIGS. 23A-23C illustrate pulmonary histopathological characteristics at 3 days after SARS-CoV-2 challenge in vaccinated hamsters. Lung sections from hamsters at 3 days after SARS-CoV-2 challenge were stained with H&E. Representative images are shown for mock-vaccinated, intranasally vaccinated (25 μg), or intramuscularly immunized (1 μg) hamsters. FIG. 23A shows moderate, interstitial infiltration by mixed inflammatory cells within alveolar walls, multifocal deposits of fibrin, and alveolar hemorrhage in the pulmonary parenchyma. FIG. 23B shows airways, including bronchi and bronchioles, which were frequently obstructed by high numbers of neutrophils in mock-vaccinated hamsters. The suppurative inflammation was not observed in vaccinated hamsters. FIG. 23C shows vascular and perivascular mixed cell infiltrates observed in medium to large-sized blood vessels. Decreased severity of vascular inflammation was observed in vaccinated hamsters. Scale bars represent 100 μm. H&E, hematoxylin and eosin; IN, intranasal; LNP, lipid nanoparticle; mRNA, messenger RNA; SARS-CoV-2, severe acute respiratory syndrome coronavirus 2.

FIGS. 24A-24B illustrate immunohistochemistry for SARS-CoV-2 nucleocapsid (N) protein in lungs after SARS-Cov-2 challenge. Lung sections from hamsters necropsied at 3 and 14 days after SARS-CoV-2 challenge were stained with an antibody raised against the SARS-CoV-2 nucleocapsid protein (N Protein). FIG. 24A shows representational images lungs from mock-vaccinated, intranasally vaccinated (mRNA-LNP1 or mRNA-LNP2 [5 μg and 25 μg]), or intramuscularly vaccinated (0.4 μg and 1 μg) hamsters. Arrowheads designate areas of positive signal within tissue. FIG. 24B shows quantification of N-protein+ cells by vaccine group. Scale bars represent 200 μm. N=5 animals per group.

FIGS. 25A-25B show viral load as determined via qRT-PCR through 14 days after SARS-CoV-2 challenge in vaccinated hamsters. Viral loads (sgRNA copies per gram of tissue) at 3 days and 14 days after SARS-CoV-2 challenge in lungs (FIG. 25A) and nasal turbinates (FIG. 25B) of vaccinated hamsters are shown. Animal-level data are shown as dots (n=5 animals per group), with the grey lines representing the geometric mean of each group. LLOD=10 copies/g of tissue. IM, intramuscular; IN, intranasal; LNP, lipid nanoparticle; mRNA, messenger RNA; qRT-PCR, quantitative reverse transcription polymerase chain reaction; SARS-CoV-2, severe acute respiratory syndrome coronavirus 2; SEM, standard error of the mean; sgRNA, subgenomic RNA.

FIGS. 26A-26C show pulmonary pathology characteristics at 14 days after SARS-CoV-2 challenge in vaccinated hamsters. Lung sections from hamsters at 14 days after SARS-CoV-2 challenge were stained with H&E. Representative images of interstitial inflammation (FIG. 26A), type II pneumocyte hyperplasia (arrows) (FIG. 26B), or airways and blood vessels (FIG. 26C) are shown for hamsters intranasally administered 2 doses of Tris/sucrose buffer (mock-vaccinated), mRNA-LNP1 (25 g), mRNA-LNP2 (25 μg), or intramuscularly vaccinated with 2 doses of vaccine (1.0 μg). Scale bars=100 μm. H&E, hematoxylin and eosin; IN, intranasal; SARS-CoV-2, severe acute respiratory syndrome coronavirus 2.

FIGS. 27A-27C show anti-gB (HSV) IgA titers at day 36 following intranasal (IN) and intramuscular administration (see Example 22). Reciprocal endpoint titers from sera (FIG. 27A), female genital tract (FGT) (FIG. 27B), and bronchoalveolar lavage (BAL) fluid (FIG. 27C) are shown.

FIG. 28 shows anti-gC (HSV) IgA titers at day 36 following intranasal (IN) and intramuscular administration (see Example 22). Reciprocal endpoint titers from sera (top graph), female genital tract (FGT) (middle graph), and bronchoalveolar lavage (BAL) fluid (bottom graph) are shown.

FIG. 29 shows anti-gD (HSV) IgA titers at day 36 following intranasal (IN) and intramuscular administration (see Example 22). Reciprocal endpoint titers from sera (top graph), female genital tract (FGT) (middle graph), and bronchoalveolar lavage (BAL) fluid (bottom graph) are shown.

### DETAILED DESCRIPTION

Despite substantial progress, disease caused by respiratory pathogens remains a preeminent threat to global public health. Lower respiratory tract infections caused an estimated 2.4 million deaths worldwide among individuals of all ages in 2016, primarily due to Streptococcus pneumoniae, respiratory syncytial virus (RSV), Haemophilus influenzae type B, and influenza virus. Further, there remains a risk for emerging infectious diseases, as highlighted by the ongoing coronavirus disease 2019 (COVID-19) pandemic caused by the severe acute respiratory syndrome coronavirus 2 (SARS-CoV-2), which is attributable to at least 587.3 million global cases and 6.5 million deaths. Vaccination remains a strategy to address respiratory infectious disease-related morbidity and mortality, and innovative immunization strategies and technologies that can establish local immunity at a key site of infection, the mucous membranes of the respiratory tract, have potential to further address the global burden of infectious disease caused by respiratory pathogens.

Most licensed vaccines are administered intramuscularly, which can induce robust systemic immunity, but can be generally poor at eliciting local or durable immunity at upper respiratory mucosal sites. Therefore, an alternative or additional preventative approach to respiratory pathogens is mucosal administration, such as intranasal immunization, which may advantageously also induce mucosal immunity to neutralize respiratory pathogens and limit infection and minimize transmission. In addition, the approach could also increase vaccination coverage, as it is minimally invasive and may facilitate self-dosing and administration without the need for a trained healthcare professional, and could bypass injection injury phobias that are a known predictor for vaccine hesitancy.

The messenger RNA (mRNA) vaccine platform has demonstrated potential for protection against infectious respiratory pathogens, as shown by mRNA-1273 (Spikevax; Moderna Inc., Cambridge, MA, USA), a lipid nanoparticle (LNP) encapsulated SARS-CoV-2 vaccine with an acceptable safety profile and high efficacy and effectiveness against symptomatic disease, hospitalization, and death. Compared with more traditional platforms, the mRNA platform has several advantages, including a flexible antigen design that eliminates vector-specific immune responses, with rapid and scalable production that can be translated across respiratory disease platforms. Further, as a delivery system, LNPs have potential for targeted delivery of mRNA to specific cells, tissues, and organs.

As described herein, an intranasally administered messenger RNA (mRNA)-lipid nanoparticle (LNP) encapsulated vaccine was found to be immunogenic and protective against a respiratory virus in Syrian golden hamsters (Examples 26 and 29). An intranasally administered mRNA-based vaccine formulated with pulmonary optimized LNP induced significantly higher immune responses than the same mRNA-based vaccine formulated with an alternative LNP composition. Further, the intranasally administered mRNA-LNP elicited similar immune responses as intramuscular administration. After viral challenge, viral loads were lower in the respiratory tracts of animals immunized with the intranasally administered mRNA-LNP or intramuscularly immunized than with placebo. Both intranasally and intramuscularly immunized animals were protected against viral pathology in the lungs.

Thus, the present disclosure, in some aspects, provides LNPs for the delivery of polynucleotide payloads to, or through, the mucosa (e.g., airway epithelial cells). For example, such LNPs can be used to deliver payloads, including nucleic acids, e.g., mRNA vaccines encoding one or more antigens or mRNA encoding therapeutic peptides to, or through, the mucosa (e.g., airway epithelial cells). Formulations comprising the nanoparticles described herein have been shown herein to be muco-penetrant, passing through the protective mucous layer of mucosal tissue to reach underlying cells that can translate their respective payloads. As is shown herein, the mucosal delivery of polynucleotide payloads using the nanoparticles effectively delivers active agent locally and systemically to produce a response. For instance, delivery of mRNA vaccines in the nanoparticles promotes protective and durable mucosal and systemic immunity.

LNPs are useful for the safe and effective delivery of payload molecules, e.g., mRNA encoding at least one antigen or therapeutic peptide, to target cells. LNPs have the unique ability to deliver nucleic acids by a mechanism involving cellular uptake, intracellular transport and endosomal release or endosomal escape. Some embodiments provided herein feature LNPs that have improved properties. In some embodiments, the LNP provided herein comprises a lipid nanoparticle core, a polynucleotide or polypeptide payload encapsulated within the core for delivery into a cell, and a cationic agent disposed primarily on the outer surface of the nanoparticle. Without being bound by a particular theory, LNPs having a cationic agent disposed primarily on the outer surface of the core can improve accumulation of the LNP in cells such as human bronchial epithelial (HBE) and also improve function of the payload molecule, e.g., as measured by mRNA expression in cells, e.g., mucosal cells and/or expression in cells underlying the mucosa.

In some aspects, provided herein is a composition, comprising a polynucleotide payload and a nanoparticle, wherein the nanoparticle has a greater than neutral zeta potential at physiologic pH, wherein the nanoparticle comprises a lipid nanoparticle core and the payload, and a cationic agent dispersed primarily on the outer surface of the core.

In some aspects, provided herein is a composition, comprising a polynucleotide or polypeptide payload and a nanoparticle, wherein the nanoparticle comprises a lipid nanoparticle core comprising an ionizable lipid, a phospholipid, a structural lipid, a PEG-lipid, and the payload, and a cationic agent dispersed primarily on the outer surface of the core.

In some aspects, provided herein is a polynucleotide or polypeptide payload and a nanoparticle, wherein the nanoparticle comprises:
(a) a lipid nanoparticle core comprising:
   (i) an ionizable lipid,
   (ii) a phospholipid,
   (iii) a structural lipid, and
   (iv) a PEG-lipid, and
(b) the payload encapsulated within the core for delivery into a cell, and
(c) a cationic agent disposed primarily on the outer surface of the core.

In one aspect, provided herein is a polynucleotide payload and a nanoparticle, wherein the nanoparticle comprises:
(a) a lipid nanoparticle core,
(b) the polynucleotide payload is encapsulated within the core for delivery into a cell, and
(c) a cationic agent,
wherein the nanoparticle exhibits a cellular accumulation of at least about 20% of cells and exhibits about 5% or greater expression in cells. In some embodiments, the nanoparticle exhibits a cellular accumulation of about 1% to about 75%, 5% to about 50%, about 10% to about 40%, or about 15% to about 25% of cells. In some embodiments, the nanoparticle exhibits about 0.5% to about 50%, about 1% to about 40%, about 3% to about 20%, or about 5% to about 15% expression in cells.

In one aspect, provided herein is a polynucleotide payload and a nanoparticle comprising:
(a) a lipid nanoparticle core,
(b) the polynucleotide payload is encapsulated within the core for delivery into a cell, and
(c) a cationic agent disposed primarily on the outer surface of the core.

In individual aspects the payload nanoparticle exhibits any one or more or all of:
(i) a cellular accumulation of at least about 20% of cells and exhibits about 5% or greater expression in cells. In some embodiments, the nanoparticle exhibits a cellular accumulation of about 1% to about 75%, 5% to about 50%, about 10% to about 40%, or about 15% to about 25% of cells. In some embodiments, the nanoparticle exhibits about 0.5% to about 50%, about 1% to about 40%, about 3% to about 20%, or about 5% to about 15% expression in cells,
(ii) nucleic acid expression of about 0.5% to 50% in cells. In some embodiments, the nanoparticle exhibits antigen expression of about 0.1% to about 60%, about 0.5% to about 40%, about 1% to about 30%, or about 1% to about 20% in cells,
(iii) nucleic acid expression of about 0.5% to 50% in cells. In some embodiments, the nanoparticle exhibits antigen expression of about 0.1% to about 60%, about 0.5% to about 40%, about 1% to about 30%, or about 1% to about 20% in cells,
(iv) a cellular accumulation of at least about 20% in mucosal cells and exhibits about 5% or greater expression in mucosal cells. In some embodiments, the nanoparticle exhibits a cellular accumulation of about 1% to about 75%, 5% to about 50%, about 10% to about 40%, or about 15% to about 25% of mucosal cells. In some embodiments, the nanoparticle exhibits about 0.5% to about 50%, about 1% to about 40%, about 3% to about 20%, or about 5% to about 15% expression in mucosal cells (in some embodiments, the mucosal cells are HBE cells),
(v) nucleic acid expression of about 0.5% to 50% of mucosal cells. In some embodiments, the nanoparticle exhibits antigen expression of about 0.1% to about 60%, about 0.5% to about 40%, about 1% to about 30%, or about 1% to about 20% of mucosal cells,
(vi) nucleic acid expression in about 0.5% to about 50% of nasal cells,
(vii) nucleic acid expression of about 0.1% to about 60%, about 0.5% to about 40%, about 1% to about 30%, or about 1% to about 20% of nasal cells,
(viii) nucleic acid expression in about 0.5% to about 50% of macrophages. In some embodiments, the nanoparticle exhibits antigen expression of about 0.1% to about 60%, about 0.5% to about 40%, about 1% to about 30%, or about 1% to about 20% of macrophages,
(ix) nucleic acid expression in about 0.5% to about 50% of HeLa cells. In some embodiments, the nanoparticle exhibits antigen expression of about 0.1% to about 60%, about 0.5% to about 40%, about 1% to about 30%, or about 1% to about 20% of HeLa cells.

In some embodiments, the cells referred to herein-above and herein-throughout can be in vitro cells or in vivo cells. In some embodiments, the cells are in vitro cells. In some embodiments, the cells are in vivo cells.

In some embodiments, the nanoparticles of the invention have increased cellular accumulation (e.g., in mucosal cells, such as airway epithelial cells) relative to nanoparticles of the substantially the same composition but prepared without post addition of the cationic agent (e.g., layering or contacting of the cationic agent with the pre-formed lipid nanoparticle). In some embodiments, the nanoparticles of the invention have increased cellular expression (e.g., in mucosal cells, such as airway epithelial cells) relative to nanoparticles of the substantially the same composition but prepared without post addition of the cationic agent (e.g., layering or contacting of the cationic agent with the pre-formed lipid nanoparticle).

In some embodiments, a weight ratio of the cationic agent to polynucleotide (e.g., mRNA) is about 0.1:1 to about 15:1. In some embodiments, a weight ratio of the cationic agent to polynucleotide is about 0.2:1 to about 10:1. In some embodiments, a weight ratio of the cationic agent to polynucleotide is about 1:1 to about 10:1. In some embodiments, a weight ratio of the cationic agent to polynucleotide is about 1:1 to about 8:1. In some embodiments, a weight ratio of the cationic agent to polynucleotide is about 1:1 to about 7:1. In some embodiments, a weight ratio of the cationic agent to polynucleotide is about 1:1 to about 6:1. In some embodiments, a weight ratio of the cationic agent to polynucleotide is about 1:1 to about 5:1. In some embodiments, a weight ratio of the cationic agent to polynucleotide is about 1:1 to about 4:1. In some embodiments, a weight ratio of the cationic agent to polynucleotide is about 1.25:1 to about 3.75:1. In some embodiments, a weight ratio of the cationic agent to polynucleotide is about 1.25:1. In some embodiments, a weight ratio of the cationic agent to polynucleotide is about 2.5:1. In some embodiments, a weight ratio of the cationic agent to polynucleotide is about 3.75:1.

In some embodiments, a molar ratio of the cationic agent to polynucleotide (e.g., mRNA) is about 0.1:1 to about 20:1. In some embodiments, a molar ratio of the cationic agent to polynucleotide is about 1.5:1 to about 10:1. In some embodiments, a molar ratio of the cationic agent to polynucleotide is about 1.5:1 to about 9:1. In some embodiments, a molar ratio of the cationic agent to polynucleotide is about 1.5:1 to about 8:1. In some embodiments, a molar ratio of the cationic agent to polynucleotide is about 1.5:1 to about 7:1. In some embodiments, a molar ratio of the cationic agent to polynucleotide is about 1.5:1 to about 6:1. In some embodiments, a molar ratio of the cationic agent to polynucleotide is about 1.5:1 to about 5:1. In some embodiments, a molar ratio of the cationic agent to polynucleotide is about 1.5:1. In some embodiments, a molar ratio of the cationic agent to polynucleotide is about 2:1. In some embodiments, a molar ratio of the cationic agent to polynucleotide is about 3:1. In some embodiments, a molar ratio of the cationic agent to polynucleotide is about 4:1. In some embodiments, a molar ratio of the cationic agent to polynucleotide is about 5:1.

In some embodiments, the nanoparticle of the invention has a zeta potential of about 5 mV to about mV. In some embodiments, the nanoparticle has a zeta potential of about 5 mV to about 15 mV. In some embodiments, the nanoparticle has a zeta potential of about 5 mV to about 10 mV.

Zeta potential measures the surface charge of colloidal dispersions. The magnitude of the zeta potential indicates the degree of electrostatic repulsion between adjacent, similarly charged particles in the dispersion. Zeta potential can be measured on a Wyatt Technologies Mobius Zeta Potential instrument. This instrument characterizes the mobility and zeta potential by the principle of “Massively Parallel Phase Analysis Light Scattering” or MP-PALS. This measurement is more sensitive and less stress inducing than ISO Method 13099-1:2012 which only uses one angle of detection and required higher voltage for operation. In some embodiments, the zeta potential of the herein described empty lipid nanoparticle compositions lipid is measured using an instrument employing the principle of MP-PALS. Zeta potential can be measured on a Malvern Zetasizer (Nano ZS).

In some embodiments, the lipid nanoparticle core has a neutral charge at a neutral pH.

In some embodiments, greater than about 80% of the cationic agent is on the surface on the nanoparticle. In some embodiments, greater than about 90% of the cationic agent is on the surface on the nanoparticle. In some embodiments, greater than about 95% of the cationic agent is on the surface on the nanoparticle.

In some embodiments, at least about 50% of the polynucleotide (e.g., mRNA) is encapsulated within the core. In some embodiments, at least about 75% of the polynucleotide or polypeptide payload is encapsulated within the core. In some embodiments, at least about 90% of the polynucleotide is encapsulated within the core. In some embodiments, at least about 95% of the polynucleotide is encapsulated within the core.

In some embodiments, the nanoparticle has a polydispersity value of less than about 0.4. In some embodiments, the nanoparticle has a polydispersity value of less than about 0.3. In some embodiments, the nanoparticle has a polydispersity value of less than about 0.2.

In some embodiments, the nanoparticle has a mean diameter of about 40 nm to about 150 nm. In some embodiments, the nanoparticle has a mean diameter of about 50 nm to about 100 nm. In some embodiments, the nanoparticle has a mean diameter of about 60 nm to about 120 nm. In some embodiments, the nanoparticle has a mean diameter of about 60 nm to about 100 nm. In some embodiments, the nanoparticle has a mean diameter of about 60 nm to about 80 nm.

In some embodiments, a general polarization of laurdan (2-dimethylamino-6-lauroylnaphtalene) of the nanoparticle is greater than or equal to about 0.6. In some embodiments, the nanoparticle has a d-spacing of greater than about 6 nm. In some embodiments, the nanoparticle has a d-spacing of greater than about 7 nm.

In some embodiments, at least 50% of the nanoparticles have a surface fluidity value of greater than a threshold polarization level. In some embodiments, at least 75% of the nanoparticles have a surface fluidity value of greater than a threshold polarization level. In some embodiments, at least 90% of the nanoparticles have a surface fluidity value of greater than a threshold polarization level. In some embodiments, at least 95% of the nanoparticles have a surface fluidity value of greater than a threshold polarization level.

In some embodiments, about 10% or greater of cell population has accumulated the nanoparticle when the nanoparticle is contacted with a population of cells. In some embodiments, about 15% or greater of cell population has accumulated the nanoparticle when the nanoparticle is contacted with a population of cells. In some embodiments, about 20% or greater of cell population has accumulated the nanoparticle when the nanoparticle is contacted with a population of cells. In some embodiments, about 5% or greater of cell expresses the polynucleotide or polypeptide when the nanoparticle is contacted with a population of cells. In some embodiments, about 10% or greater of cell expresses the polynucleotide or polypeptide when the nanoparticle is contacted with a population of cells. In some embodiments, the cell population is a mucosal cell population. In some embodiments, the cell population is an epithelial cell population. In some embodiments, the cell population is a respiratory epithelial cell population. In some embodiments, the respiratory epithelial cell population is a nasal cell population. In some embodiments, the cell population is a nasal cell population. In some embodiments, the cell population is HeLa population.

### Cationic Agent

The cationic agent can comprise any aqueous soluble molecule or substance that has a net positive charge at physiological pH and can adhere to the surface of a lipid nanoparticle core. Such agent may also be lipid soluble but will also be soluble in aqueous solution. The cationic agent can be charged at physiologic pH. Physiological pH is the pH level normally observed in the human body. Physiological pH can be about 7.30-7.45 or about 7.35-7.45. Physiological pH can be about 7.40. Generally speaking, the cationic agent features a net positive charge at physiologic pH because it contains one or more basic functional groups that are protonated at physiologic pH in aqueous media. For example, the cationic agent can contain one or more amine groups, e.g. primary, secondary, or tertiary amines each having a pKa of 8.0 or greater. The pKa can be greater than about 9. The pKa can be from 9.5-11.0, inclusive.

In some embodiments, the cationic agent can be a cationic lipid which is a water-soluble, amphiphilic molecule in which one portion of the molecule is hydrophobic comprising, for example, a lipid moiety, and where the other portion of the molecule is hydrophilic, containing one or more functional groups which are typically charged at physiologic pH. The hydrophobic portion, comprising the lipid moiety, can serve to anchor the cationic agent to a lipid nanoparticle core. The hydrophilic portion can serve to increase the charge on the surface of a lipid nanoparticle core. For example, the cationic agent can have a solubility of greater than about 1 mg/mL in alcohol. The solubility in alcohol can be greater than about 5 mg/mL. The solubility in alcohol can be greater than about 10 mg/mL. The solubility in alcohol can be greater than about 20 mg/mL in alcohol. The alcohol can be C₁₋₆ alcohol such as ethanol.

The lipid portion of the molecule can be, for example, a structural lipid, fatty acid, or similar hydrocarbyl group.

The structural lipid can be selected from, but is not limited to, a steroid, diterpeniod, triterpenoid, cholestane, ursolic acid, and derivatives thereof.

In some embodiments, the structural lipid is a steroid selected from, but not limited to, cholesterol or a phystosterol. In some embodiments, the structural lipid is an analog of cholesterol. In some embodiments, the structural lipid is a sitosterol, campesterol, or stigmasterol. In some embodiments, the structural lipid is an analog of sitosterol, campesterol, or stigmasterol. In some embodiments, the structural lipid is β-sitosterol.

The fatty acid comprises 1 to 4 C₆₋₂₀ hydrocarbon chains. The fatty acid can be fully saturated or can contain 1 to 7 double bonds. The fatty acid can contain 1 to 5 heteroatoms either along the main chain or pendent to the main chain.

In some embodiments, the fatty acid comprises two C₁₀₋₁₈ hydrocarbon chains. In some embodiments, the fatty acid comprises two C₁₀₋₁₈ saturated hydrocarbon chains. In some embodiments, the fatty acid comprises two C₁₆ saturated hydrocarbon chain. In some embodiments, the fatty acid comprises two C₁₄ saturated hydrocarbon chain. In some embodiments, the fatty acid comprises two unsaturated C₁₀₋₁₈ hydrocarbon chains. In some embodiments, the fatty acid comprises two C₁₆₋₁₈ hydrocarbon chains, each with one double bond. In some embodiments, the fatty acid comprises three C₈₋₁₈ saturated hydrocarbon chains.

The hydrocarbyl group consists of 1 to 4 C₆₋₂₀ alkyl, alkenyl, or alkynyl chains or 3 to 10 membered cycloalkyl, cycloalkenyl, or cycloalkynyl groups.

In some embodiments, the hydrocarbyl group is a C₈₋₁₀ alkyl. In some embodiments, the hydrocarbyl group is C₈₋₁₀ alkenyl.

The hydrophilic portion can comprise 1 to 5 functional groups that would be charged at physiologic pH, 7.3 to 7.4. The hydrophilic group can comprise a basic functional group that would be protonated and positively charged at physiologic pH. At least one of the basic functional groups has a pKa of 8 or greater. In some embodiments, at least one of the basic functional groups has a pKa of 9 or greater. In some embodiments, at least one of the basic functional groups has a pKa of 9.5 to 11.

In some embodiments, the hydrophilic portion comprises an amine group. The amine group can comprise one to four primary, secondary, or tertiary amines and mixtures thereof. The primary, secondary, or tertiary amines can be part of larger amine containing functional group selected from, but not limited to, —C(═N—)—N—, —C═C—N—, —C═N—, or —N—C(═N—)—N—. The amine can be contained in a three to eight membered heteroalkyl or heteroaryl ring.

In some embodiments, the amine group comprises one or two terminal primary amines. In some embodiments, the amine group comprises one or two terminal primary amines and one internal secondary amine. In some embodiments, the amine group comprises one or two tertiary amines. In some embodiments, the tertiary amine is (CH₃)₂N—. In some embodiments, amine group comprises one to two terminal (CH₃)₂N—.

The hydrophilic portion can comprise a phosphonium group. The counterion of the phosphonium ion consists of an anion with a charge of one.

In some embodiments, three of the substituents on the phosphonium are isopropyl groups. In some embodiments, the counterion is a halo, hydrogen sulfate, nitrite, chlorate, or hydrogen carbonate. In some embodiments, the counterion is a bromide.

In some embodiments, the cationic agent is a cationic lipid which is a sterol amine. A sterol amine has, for its hydrophobic portion, a sterol, and for its hydrophilic portion, an amine group. The sterol group is selected from, but not limited to, cholesterol, sitosterol, campesterol, stigmasterol or derivatives thereof. The amine group can comprise one to five primary, secondary, tertiary amines, or mixtures thereof. At least one of the amines has a pKa of 8 or greater and is charged at physiological pH. The primary, secondary, or tertiary amines can be part of a larger amine containing functional group selected from, but not limited to —C(═N—)—N—, —C═C—N—, —C═N—, or —N—C(═N—)—N—. The amine can be contained in a three to eight membered heteroalkyl or heteroaryl ring.

In some embodiments, the amine group of the sterol amine comprises one or two terminal primary amines. In some embodiments, the amine group comprises one or two terminal primary amines and one internal secondary amine. In some embodiments, the amine group comprises one or two tertiary amines. In some embodiments, the tertiary amine is (CH₃)₂N—. In some embodiments, amine group comprises one to two terminal (CH₃)₂N—.

Sterol amines useful in the nanoparticles of the invention include molecules having Formula (A1):

A-L-B (A1)

or a salt thereof, wherein:

A is an amine group, L is an optional linker, and B is a sterol.

In some embodiments, the amine group is an alkyl (e.g., C₁₋₁₄ alkyl, C₁₋₁₂ alkyl, C₁₋₁₀ alkyl, etc.), 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl), wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof, wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, —OH, —O(C₁₋₆ alkyl), —C₁₋₆ alkyl-OH, —NH₂, —NH(C₁_s alkyl), —N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, —NH(3 to 8 membered heterocycloalkyl), and —NH(5 to 6 membered heteroaryl). In some embodiments, the linker is absent, —O—, —S—S—, —OC(═O), —C(═O)N—, —OC(═O)N—, —CH₂—NH—C(O)—, —C(O)O—, —OC(O)—CH₂—CH₂—C(═O)N—, —S—S—CH₂—, or —SS—CH₂—CH₂—C(O)N—. In some embodiments, the sterol group is a cholesterol, sitosterol, campesterol, stigmasterol or derivatives thereof.

In some embodiments, the sterol amine has Formula A2a:

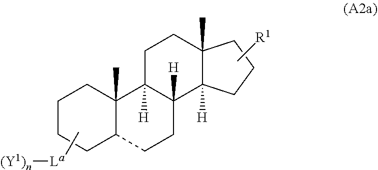

or a salt thereof, wherein:
[symbol] is a single or double bond
R¹ is C₁₋₁₄ alkyl or C₁₋₁₄ alkenyl;
L^{a} is absent, —O—, —S—S—, —OC(═O)—, —C(═O)N—, —OC(═O)N—, CH₂—NH—C(O)—, —C(O)O—, —OC(O)—CH₂—CH₂—C(═O)N—, —S—S—CH₂, —SS—CH₂—CH₂—C(O)N—, or a group of formula (a):

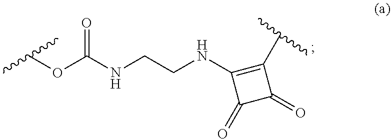 [SMILES: *OC(=O)NCCNc1c(*)c(=O)c1=O]

Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl)
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, —OH, —O(C₁₋₆ alkyl), —C₁₋₆ alkyl-OH, —NH₂, —NH(C₁₋₆ alkyl), —N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, —NH(3 to 8 membered heterocycloalkyl), and —NH(5 to 6 membered heteroaryl); and
n=1 or 2.

In some embodiments, n=1.

In some embodiments, the sterol amine has Formula A2a with the proviso that the compound of Formula A2a is other than: SA1, SA2, SA3, SA4, SA5, SA6, SA7, SA8, SA9, SA10, SA11, SA22, SA23, SA29, SA30, SA39, and SA40.

In some embodiments, ---- is a double bond. In some embodiments, ---- is a single bond.

In some embodiments, L^{a} is —OC(═O)—, —OC(═O)N—, or —OC(═O)—CH₂—CH₂—C(═O)N—.

In some embodiments, n is 1. In some embodiments, n is 2.

In some embodiments, R¹ is C₁₋₁₄ alkyl. In some embodiments, R¹ is C₁₋₁₄ alkenyl. In some embodiments, R¹ is

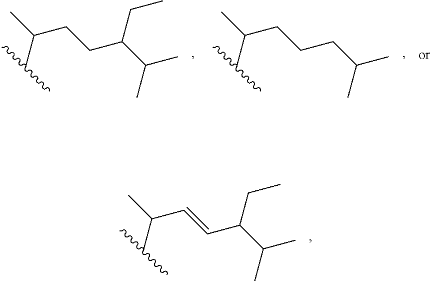

In some embodiments, Y¹ is C₁₋₁₀ alkyl, 3 to 8-membered heterocycloalkyl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, C₁₋₆ alkyl-(3 to 8-membered heterocycloalkyl), and C₁₋₆ alkyl-(5 to 6-membered heteroaryl) are each optionally substituted with C₁₋₆ alkyl, —OH, —C₁₆ alkyl-OH, or —NH₂.

In some embodiments, the sterol amine has Formula A2:

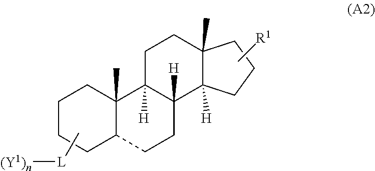

or a salt thereof, wherein:
[symbol] is a single or double bond
R¹ is C₁₋₁₄ alkyl or C₁₋₁₄ alkenyl;
L is absent, —O—, —S—S—, —OC(═O)—, —C(═O)N—, —OC(═O)N—, —CH₂—NH—C(O)—, —C(O)O—, —OC(O)—CH₂—CH₂—C(═O)N—, —S—S—CH₂, or —SS—CH₂—CH₂—C(O)N—;
Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, —OH, —O(C₁₋₆ alkyl), —C₁₋₆ alkyl-OH, —NH₂, —NH(C₁₋₆ alkyl), —N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, —NH(3 to 8 membered heterocycloalkyl), and —NH(5 to 6 membered heteroaryl); and n=1 or 2. In some embodiments, n=1.

In some embodiments, the sterol amine has Formula A3a:

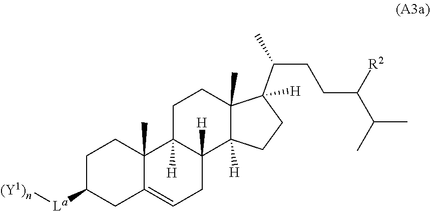

or a salt thereof, wherein:
[symbol] is a single or double bond;
R² is H or C₁₋₆ alkyl;
L^{a} is absent, —O—, —S—S—, —OC(═O)—, —C(═O)N—, —OC(═O)N—, —CH₂—NH—C(O)—, —C(O)O—, —OC(O)—CH₂—CH₂—C(═O)N—, —S—S—CH₂, —SS—CH₂—CH₂—C(O)N—, or a group of formula (a):

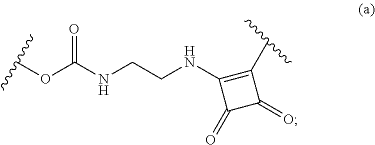 [SMILES: *OC(=O)NCCNc1c(*)c(=O)c1=O]

Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, —OH, —O(C₁₋₆ alkyl), —C₁₋₆ alkyl-OH, —NH₂, —NH(C₁₋₆ alkyl), —N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, —NH(3 to 8 membered heterocycloalkyl), and —NH(5 to 6 membered heteroaryl); and
n=1 or 2.
   In some embodiments, n=1.

In some embodiments, the sterol amine has Formula A3a with the proviso that the compound of Formula A3a is other than: SA1, SA2, SA3, SA4, SA5, SA9, SA10, SA11, SA22, SA23, SA29, SA30, SA39, and SA40.

In some embodiments, [symbol] is a double bond. In some embodiments, [symbol] is a single bond.

In some embodiments, L^{a} is —OC(═O)—, —OC(═O)N—, or —OC(═O)—CH₂—CH₂—C(═O)N—. In some embodiments, n is 1. In some embodiments, n is 2. In some embodiments, R² is H. In some embodiment, R² is ethyl.

In some embodiments, Y¹ is C₁₋₁₀ alkyl, 3 to 8-membered heterocycloalkyl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, C₁₋₆ alkyl-(3 to 8-membered heterocycloalkyl), and C₁₋₆ alkyl-(5 to 6-membered heteroaryl) are each optionally substituted with C₁₋₆ alkyl, —OH, —C₁₋₆alkyl-OH, or —NH₂.

In some embodiments, the sterol amine has Formula A3:

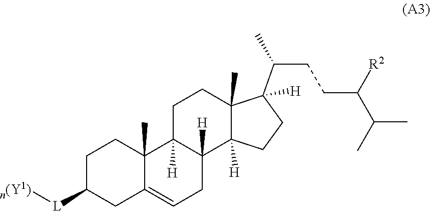

or a salt thereof, wherein:
[symbol] is a single or double bond;
R² is H or C₁₋₆ alkyl;
L is absent, —O—, —S—S—, —OC(═O)—, —C(═O)N—, —OC(═O)N—, CH₂—NH—C(O)—, —C(O)O—, —OC(O)—CH₂—CH₂—C(═O)N—, —S—S—CH₂, or —SS—CH₂—CH₂—C(O)N—;
Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, —OH, —O(C₁₋₆alkyl), —C₁₋₆alkyl-OH, —NH₂, —NH(C₁₋₆ alkyl), —N(C₁₋₆ alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, —NH(3 to 8 membered heterocycloalkyl), and —NH(5 to 6 membered heteroaryl); and
n=1 or 2. In some embodiments, n=1.

In some embodiments, Y¹ is selected from:

(1)

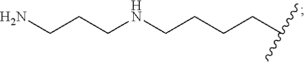 [SMILES: *CCCCNCCCN]

(2)

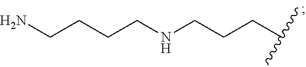 [SMILES: *CCCNCCCCN]

(3)

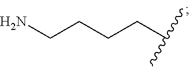 [SMILES: *CCCCN]

(4)

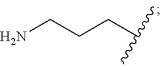 [SMILES: *CCCN]

(5)

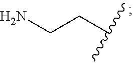 [SMILES: *CCN]

(6)

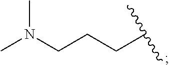 [SMILES: *CCCN(C)C]

(7)

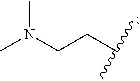 [SMILES: *CCN(C)C]

(8)

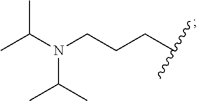 [SMILES: *CCCN(C(C)C)C(C)C]

(9)

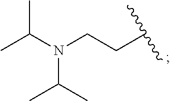 [SMILES: *CCN(C(C)C)C(C)C]

(10)

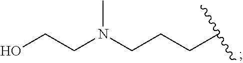 [SMILES: *CCCN(C)CCO]

(11)

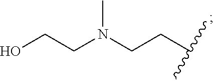 [SMILES: *CCN(C)CCO]

(12)

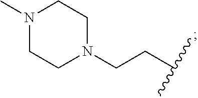 [SMILES: *CCN1CCN(C)CC1]

(13)

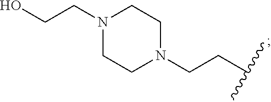 [SMILES: *CCN1CCN(CCO)CC1]

(14)

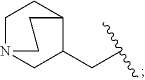 [SMILES: *CC1CN2CCC1CC2]

(15)

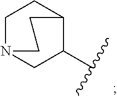 [SMILES: *C1CN2CCC1CC2]

(16)

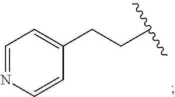 [SMILES: *CCc1ccncc1]

(17)

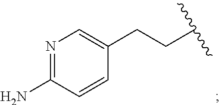 [SMILES: *CCc1ccc(N)nc1]

(18)

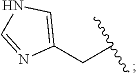 [SMILES: *Cc1c[nH]cn1]

(19)

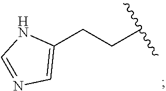 [SMILES: *CCc1cnc[nH]1]

(20)

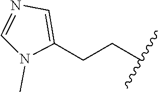 [SMILES: *CCc1cncn1C]

(21)

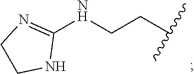 [SMILES: *CCNC1=NCCN1]

(22)

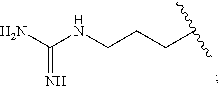 [SMILES: *CCCNC(=N)N]

(23)

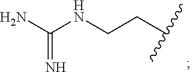 [SMILES: *CCNC(=N)N]

(28) —N(CH₃)₂; (29)

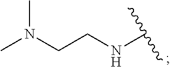 [SMILES: *NCCN(C)C]

(30)

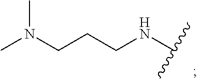 [SMILES: *NCCCN(C)C]

(31)

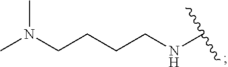 [SMILES: *NCCCCN(C)C]

and (32)

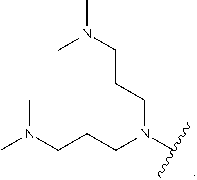 [SMILES: *N(CCCN(C)C)CCCN(C)C]

In some embodiments Y¹ is selected from:

(1)

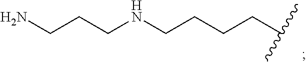 [SMILES: *CCCCNCCCN]

(2)

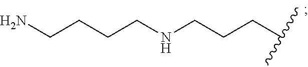 [SMILES: *CCCNCCCCN]

(3)

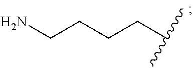 [SMILES: *CCCCN]

(4)

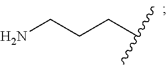 [SMILES: *CCCN]

(5)

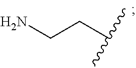 [SMILES: *CCN]

(6)

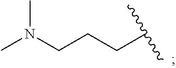 [SMILES: *CCCN(C)C]

(7)

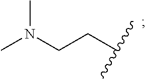 [SMILES: *CCN(C)C]

(8)

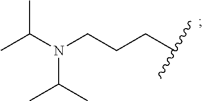 [SMILES: *CCCN(C(C)C)C(C)C]

(9)

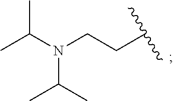 [SMILES: *CCN(C(C)C)C(C)C]

(10)

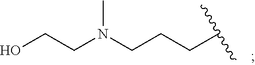 [SMILES: *CCCN(C)CCO]

(11)

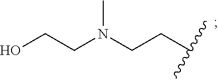 [SMILES: *CCN(C)CCO]

(12)

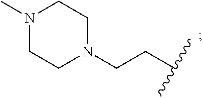 [SMILES: *CCN1CCN(C)CC1]

(13)

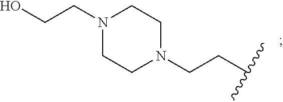 [SMILES: *CCN1CCN(CCO)CC1]

(14)

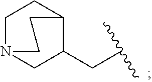 [SMILES: *CC1CN2CCC1CC2]

(15)

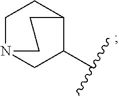 [SMILES: *C1CN2CCC1CC2 ; ";"]

(16)

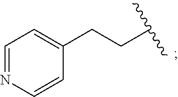 [SMILES: *CCc1ccncc1]

(17)

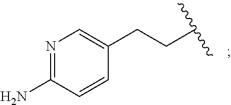 [SMILES: *CCc1ccc(N)nc1]

(18)

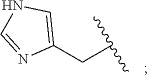 [SMILES: *Cc1c[nH]cn1]

(19)

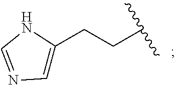 [SMILES: *CCc1cnc[nH]1]

(20)

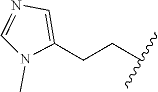 [SMILES: *CCc1cncn1C]

(21)

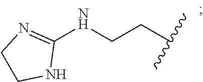 [SMILES: *CCNC1=NCCN1]

(22)

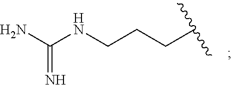 [SMILES: *CCCNC(=N)N]

(23)

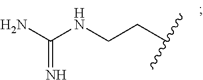 [SMILES: *CCNC(=N)N]

(24)

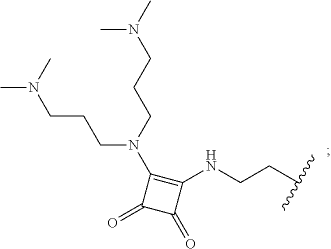 [SMILES: *CCNc1c(N(CCCN(C)C)CCCN(C)C)c(=O)c1=O]

(25)

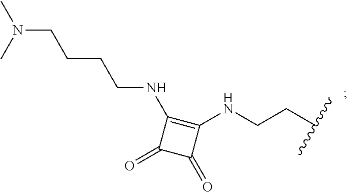 [SMILES: *CCNc1c(NCCCCN(C)C)c(=O)c1=O]

(26)

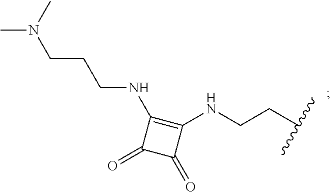 [SMILES: *CCNc1c(NCCCN(C)C)c(=O)c1=O]

(27)

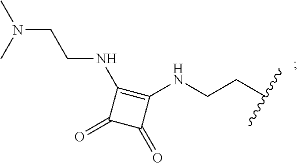 [SMILES: *CCNc1c(NCCN(C)C)c(=O)c1=O]

and (28) —N(CH₃)₂.

In some embodiments, the sterol amine has Formula A4:

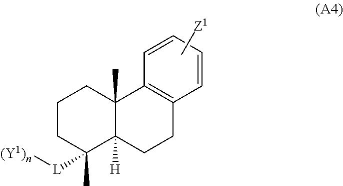

or a salt thereof, wherein:
Z¹ is —OH or C₃₋₆ alkyl;
L is absent, —O—, —S—S—, —OC(═O)—, —C(═O)N—, —OC(═O)N—, CH₂—NH—C(O)—, —C(O)O—, —OC(O)—CH₂—CH₂—C(═O)N—, —S—S—CH₂—, or —SS—CH₂—CH₂—C(O)N—;
Y¹ is C₁₋₁₀ alkyl, 3 to 8 membered heterocycloalkyl, 5 to 6 membered heteroaryl, C₁₋₆ alkyl-(3 to 8 membered heterocycloalkyl), or C₁₋₆ alkyl-(5 to 6 membered heteroaryl),
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) comprises one to five primary, secondary, or tertiary amines or combination thereof,
wherein the alkyl, 3 to 8 membered heterocycloalkyl, 5 to 6 membered heteroaryl, C₁₋₆ alkyl-(3 to 8 membered heterocycloalkyl), and C₁₋₆ alkyl-(5 to 6 membered heteroaryl) are each optionally substituted with 1, 2, 3, or 4 substituents selected from C₁₋₆ alkyl, halo, —OH, —O(C₁₋₆ alkyl), —C₁₋₆ alkyl-OH, —NH₂, —NH(C₁₋₆ alkyl), —N(C₁₋₆alkyl)₂, 3 to 8 membered heterocycloalkyl (optionally substituted with C₁₋₁₄ alkyl comprising one to five primary, secondary, or tertiary amines or combination thereof), 5 to 6 membered heteroaryl, —NH(3 to 8 membered heterocycloalkyl), and —NH(5 to 6 membered heteroaryl); and
n=1 or 2.

In some embodiments, Z¹ is —OH. In some embodiments, Z¹ is C₃₋₆ alkyl.

In some embodiments, L is —C(═O)N—, —CH₂—NH—C(═O)—, or —C(═O)O—.

In some embodiments, Y¹ is C₁₋₁₀ alkyl comprising one to five primary, secondary, or tertiary amines or combination thereof. In some embodiments, Y¹ is

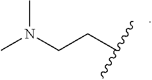 [SMILES: *CCN(C)C]

In some embodiments, n is 1. In some embodiments, n is 2.

In some embodiments, the sterol amine has Formula A5:

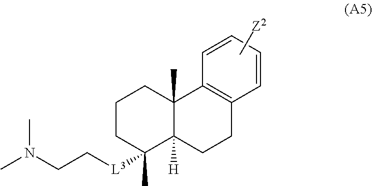

or a salt thereof, wherein:
Z² is —OH or isopropyl;
L³ is —CH₂—NH—C(O)—, —C(O)NH—, or —C(O)O—.

In some embodiments, the sterol amine has Formula A6:

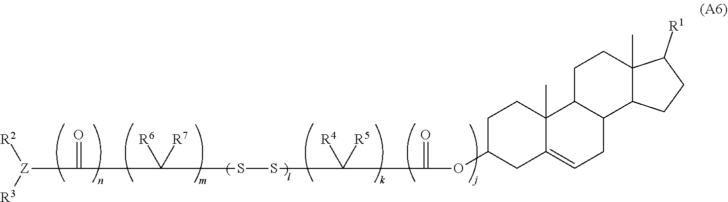

or a salt thereof, wherein:
Z is N or CH;
R¹ is C₁₋₁₄ alkyl, C₁₋₁₄ alkenyl, or C₁₋₁₄ hydroxyalkyl;
R² and R³ are each C₂ 20 alkyl, wherein:
(i) the C₂₋₂₀ alkyl is substituted by 1, 2, 3, 4, or 5 substituents independently selected from —NR⁸R⁹, —OH, and halo, wherein at least one substituent is —NR⁸R⁹;
(ii) 1, 2, 3, or 4 non-terminal carbons of the C₂ 20 alkyl are optionally replaced with —O—;
(iii) 1, 2, 3, or 4 non-terminal carbons of the C₂₋₂₀ alkyl are optionally replaced with —NR¹⁰—.
(iv) 1, 2, 3, or 4 non-terminal carbons of the C₂₋₂₀ alkyl are optionally replaced with —C(═O)—; and
(v) 1, 2, 3, or 4 non-terminal carbons of the C₂₋₂₀ alkyl are optionally replaced with —CR^{a}R^{b}— wherein R^{a} and R^{b} together with the C atom to which they are attached form a C₃₋₆ cycloalkyl group;
wherein R² and R³ are the same or different;
or R² and R³ together with the N atom to which they are attached form a 7-18 membered heterocycloalkyl group comprising 1, 2, or 3 ring-forming —NR¹⁰— groups, wherein the 7-18 membered heterocycloalkyl group is optionally substituted with 1, 2, or 3 substituents independently selected from C₁₋₄ alkyl, —NR⁸R⁹, —OH, and halo;
or R², R³, and R⁶, together with the atoms to which they are attached and any intervening atoms, form a 7-18 membered bridged heterocycloalkyl group optionally substituted with 1, 2, or 3 substituents independently selected from C₁₋₄ alkyl, —NR⁸R⁹, —OH, and halo;
R⁴, R¹, R⁶, and R⁷ are each independently selected from H, halo, and C₁₋₄ alkyl;
or R⁴ and R⁵ together with the carbon atom to which they are attached form a C₃₇ cycloalkyl group;
or R⁶ and R⁷ together with the carbon atom to which they are attached form a C₃₇ cycloalkyl group;
R⁸, R⁹, and R¹⁰ are each independently selected from H and C₁₋₄ alkyl;
j is 0 or 1;
k is 0, 1, 2, 3, 4, 5, or 6;
l is 0 or 1;
m is 0, 1, 2, 3, 4, 5, or 6; and
n is 0 or 1;
wherein when j is 0, then l is 1,
wherein j and 1 are not both 0.

In some embodiments, the compound is other than:

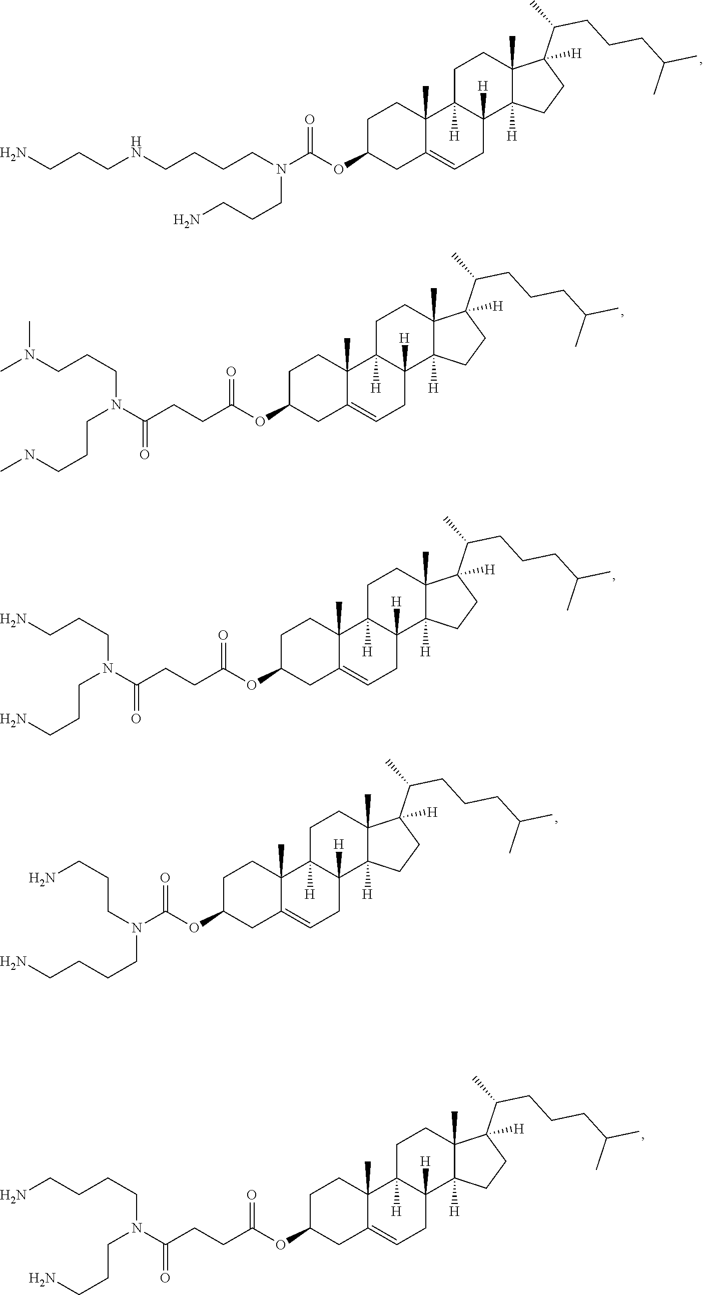

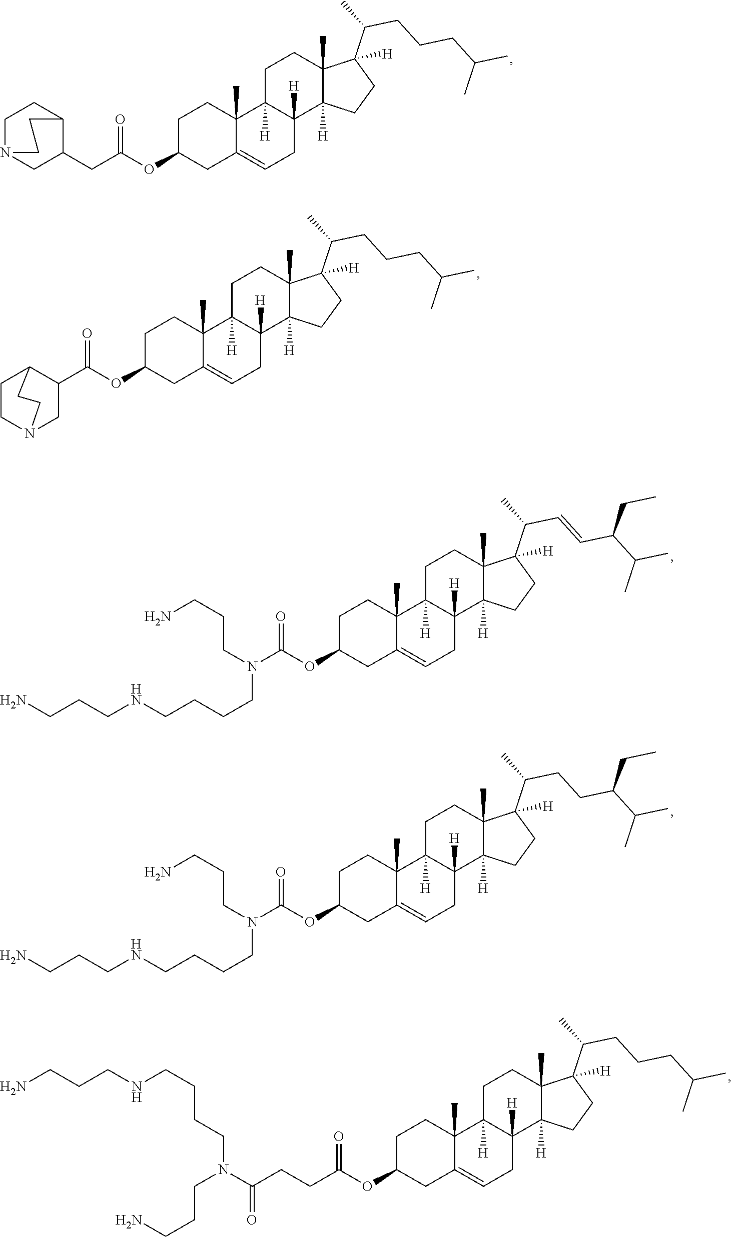

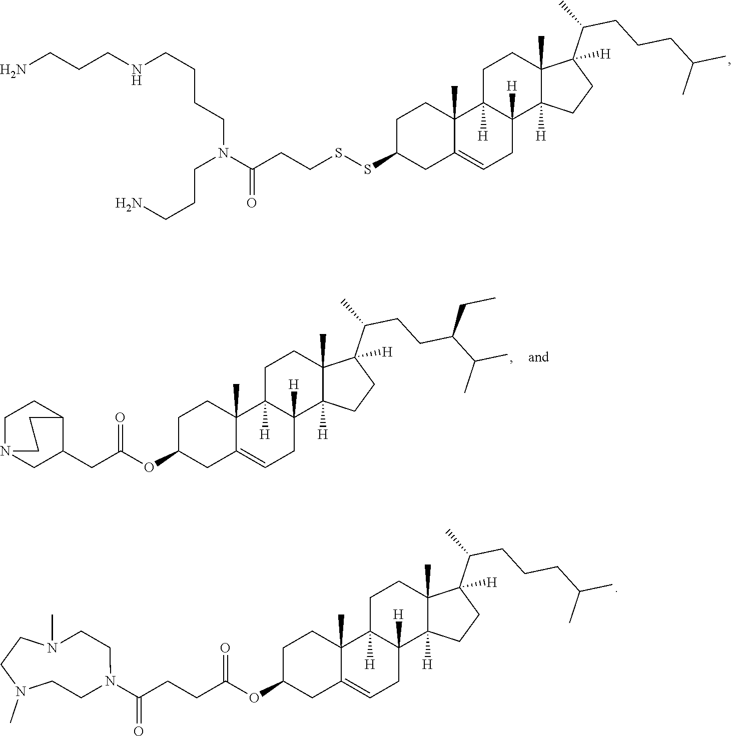

In some embodiments, Z is N. In some embodiments, Z is CH.

In some embodiments, R¹ is C₁₋₁₄ alkyl. In some embodiments, R¹ is C₃₋₁₂ alkyl. In some embodiments, R¹ is C₆₋₁₂ alkyl. In some embodiments, R¹ is C₈₋₁₀ alkyl. In some embodiments, R¹ is C₈ alkyl. In some embodiments, R¹ is C₁₀ alkyl.

In some embodiments, R¹ is C₁₋₁₄ hydroxyalkyl. In some embodiments, R¹ is C₃₋₁₂ hydroxyalkyl. In some embodiments, R¹ is C₆₋₁₂ hydroxyalkyl. In some embodiments, R¹ is C₈₋₁₀ hydroxyalkyl. In some embodiments, R¹ is C₈ hydroxyalkyl. In some embodiments, R¹ is C₁₀ hydroxyalkyl.

In some embodiments, R¹ is C₁₋₁₄ alkenyl. In some embodiments, R¹ is C₃₋₁₂ alkenyl. In some embodiments, R¹ is C₆₋₁₂ alkenyl. In some embodiments, R¹ is C₈₋₁₀ alkenyl. In some embodiments, R¹ is C₈ alkenyl. In some embodiments, R¹ is C₁₀ alkenyl.

In some embodiments, R¹ is

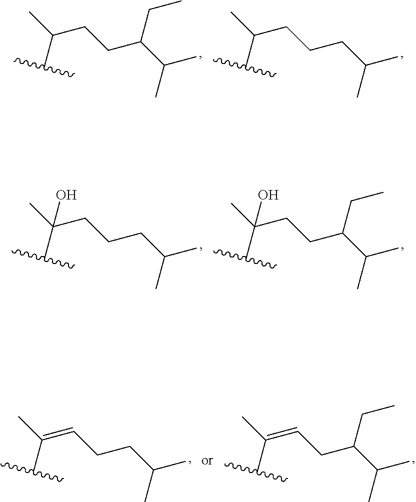

In some embodiments, R¹ is

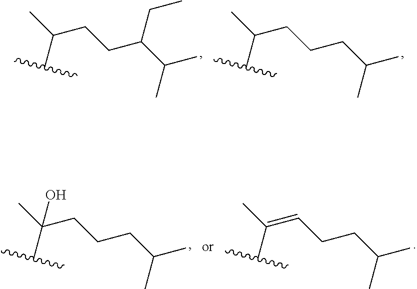

In some embodiments, R¹ is

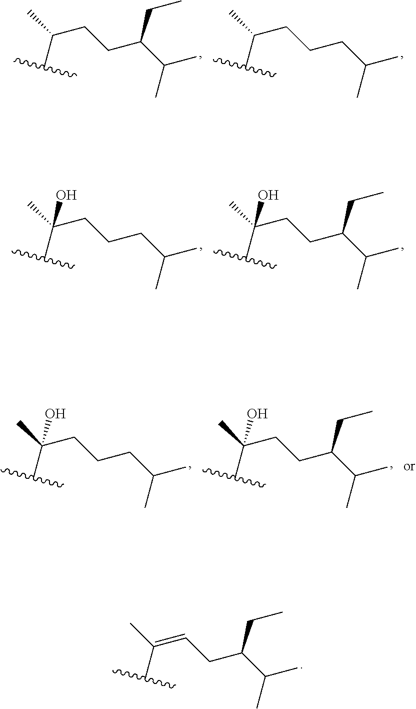

In some embodiments, when j is 1, then l is 0.

In some embodiments, when j is 0, then l is 1.

In some embodiments, when one of j and l is 1, then the other is 0.

In some embodiments, j is 0. In some embodiments, j is 1.

In some embodiments, k is 0, 1, 2, 3, or 4. In some embodiments, k is 0, 2, 3, or 4. In some embodiments, k is 0. In some embodiments, k is 1. In some embodiments, k is 2. In some embodiments, k is 3. In some embodiments, k is 4. In some embodiments, k is 5. In some embodiments, k is 6.

In some embodiments, l is 0. In some embodiments, l is 1.

In some embodiments, m is 0, 1, 2, or 4. In some embodiments, m is 0. In some embodiments, m is 1.

In some embodiments, m is 2. In some embodiments, m is 3. In some embodiments, m is 4. In some embodiments, m is 5. In some embodiments, m is 6.

In some embodiments, n is 0. In some embodiments, n is 1.

In some embodiments, j is 0, k is 0, 1 is 1, m is 1, and n is 1. In some embodiments, j is 0, k is 0, 1 is 1, m is 2, and n is 1. In some embodiments, j is 0, k is 0, 1 is 1, m is 4, and n is 1. In some embodiments, j is 1, k is 0, 1 is 0, m is 0, and n is 0. In some embodiments, j is 1, k is 1, l is 0, m is 0, and n is 0. In some embodiments, j is 1, k is 1, l is 0, m is 0, and n is 1. In some embodiments, j is 1, k is 1, l is 0, m is 2, and n is 0. In some embodiments, j is 1, k is 1, l is 1, m is 1, and n is 1. In some embodiments, j is 1, k is 2, l is 0, m is 0, and n is 0. In some embodiments, j is 1, k is 2, l is 0, m is 0, and n is 1. In some embodiments, j is 1, k is 3, l is 0, m is 0, and n is 1. In some embodiments, j is 1, k is 4, l is 0, m is 0, and n is 1.

In some embodiments, k is 1 and both R⁴ and R⁵ are H. In some embodiments, k is 1 and one of R⁴ and R⁵ is C₁₋₄ alkyl and the other of R⁴ and R⁵ is H. In some embodiments, k is 1 and one of R⁴ and R⁵ is methyl and the other of R⁴ and R⁵ is H. In some embodiments, k is 2 and each R⁴ and R⁵ is H. In some embodiments, k is 2 and one R⁴ is C₁₄ alkyl and the remaining R⁴ and R⁵ substituents are H. In some embodiments, k is 2 and one R⁴ is methyl and the remaining R⁴ and R¹ substituents are H. In some embodiments, k is 3 and each R⁴ and R¹ is H. In some embodiments, k is 4 and each R⁴ and R¹ is H.

In some embodiments, m is 1 and both R⁶ and R⁷ are H. In some embodiments, m is 2 and each R⁶ and R⁷ is H. In some embodiments, m is 4 and each R⁶ and R⁷ is H. In some embodiments, m is 2, one R⁶ with R² and R³ form, together with the atoms to which they are attached and any intervening atoms, a 7-18 membered bridged heterocycloalkyl group and the other R⁶ is H, and both R⁷ are H.

In some embodiments, j is 0, k is 0, 1 is 1, m is 1, both R⁶ and R⁷ are H, and n is 1. In some embodiments, j is 0, k is 0, 1 is 1, m is 2, each R⁶ and R⁷ is H, and n is 1. In some embodiments, j is 0, k is 0, 1 is 1, m is 4, each R⁶ and R⁷ is H, and n is 1. In some embodiments, j is 1, k is 1, each R⁴ and R⁵ is H, I is 0, m is 0, and n is 0. In some embodiments, j is 1, k is 1, one of R⁴ and R⁵ is C₁₋₄ alkyl and the other of R⁴ and R⁵ is H, l is 0, m is 0, and n is 0. In some embodiments, j is 1, k is 1, each R⁴ and R⁵ is H, l is 0, m is 0, and n is 1. In some embodiments, j is 1, k is 1, one of R⁴ and R¹ is C₁₋₄ alkyl and the other of R⁴ and R⁵ is H, l is 0, m is 0, and n is 1. In some embodiments, j is 1, k is 2, each R⁴ and R⁵ is H, l is 0, m is 0, and n is 0. In some embodiments, j is 1, k is 2, one R⁴ is C₁₋₄ alkyl and the remaining R⁴ and R⁵ substituents are H, l is 0, m is 0, and n is 0. In some embodiments, j is 1, k is 2, each R⁴ and R¹ is H, l is 0, m is 0, and n is 1. In some embodiments, j is 1, k is 3, each R⁴ and R⁵ is H, l is 0, m is 0, and n is 1. In some embodiments, j is 1, k is 4, each R⁴ and R⁵ is H, l is 0, m is 0, and n is 1. In some embodiments, j is 1, k is 1, each R⁴ and R⁵ is H, l is 1, m is 1, both R⁶ and R⁷ are H, and n is 1. In some embodiments, j is 1, k is 1, each R⁴ and R¹ is H, l is 0, m is 2, one of R⁶ with R² and R³ form, together with the atoms to which they are attached and any intervening atoms, a 7-18 membered bridged heterocycloalkyl group and the other R⁶ is H, both R⁷ are H, and n is 0.

In some embodiments, j is 1, k is 1, one of R⁴ and R⁵ is methyl and the other of R⁴ and R¹ is H, l is 0, m is 0, and n is 0. In some embodiments, j is 1, k is 1, one of R⁴ and R¹ is methyl and the other of R⁴ and R⁵ is H, l is 0, m is 0, and n is 1. In some embodiments, j is 1, k is 2, one of R⁴ is methyl and the remaining R⁴ and R⁵ substituents are H, l is 0, m is 0, and n is 0.

In some embodiments, R² and R³ are each independently selected from C₂₋₁₀ alkyl, wherein the C₂₋₁₀ alkyl is substituted by 1, 2, 3, 4, or 5 substituents independently selected from —NR⁸R⁹, —OH, and halo, wherein at least one substituent is —NR⁸R⁹.

In some embodiments, R² and R³ are each independently selected from C₂₋₁₀ alkyl, wherein:
(i) the C₂₋₁₀ alkyl is substituted by 1, 2, 3, 4, or 5 substituents independently selected from —NR⁸R⁹, —OH, and halo, wherein at least one substituent is —NR⁸R⁹; and
(ii) 1, 2, 3, or 4 non-terminal carbons of the C₂₋₁₀ alkyl are optionally replaced with —O—.

In some embodiments, R² and R³ are each independently selected from C₂₋₁₀ alkyl, wherein:
(i) the C₂₋₁₀ alkyl is substituted by 1, 2, 3, 4, or 5 substituents independently selected from —NR⁸R⁹, —OH, and halo, wherein at least one substituent is —NR⁸R⁹; and
(iii) 1, 2, 3, or 4 non-terminal carbons of the C₂₋₁₀ alkyl are optionally replaced with —NR¹⁰—.

In some embodiments, R² and R³ are each independently selected from C₂₋₁₀ alkyl, wherein:
(i) the C₂₋₁₀ alkyl is substituted by 1, 2, 3, 4, or 5 substituents independently selected from —NR⁸R⁹, —OH, and halo, wherein at least one substituent is —NR⁸R⁹; and
(iv) 1, 2, 3, or 4 non-terminal carbons of the C₂₋₁₀ alkyl are optionally replaced with —C(═O)—.

In some embodiments, R² and R³ are each independently selected from C₂₋₁₀ alkyl, wherein:
(i) the C₂₋₂₀ alkyl is substituted by 1, 2, 3, 4, or 5 substituents independently selected from —NR⁸R⁹, —OH, and halo, wherein at least one substituent is —NR⁸R⁹; and
(v) 1, 2, 3, or 4 non-terminal carbons of the C₂₋₂₀ alkyl are optionally replaced with —CR^{a}R^{b}— wherein R^{a} and R^{b} together with the C atom to which they are attached form a C₃₋₆ cycloalkyl group.

In some embodiments, R² and R³ are each independently selected from C₂₋₁₀ alkyl, wherein:
(i) the C₂₋₁₀ alkyl is substituted by 1, 2, 3, or 4 substituents independently selected from —NR⁸R⁹, —OH, and halo, wherein at least one substituent is —NR⁸R⁹;
(ii) 1 or 2 non-terminal carbons of the C₂₋₁₀ alkyl are optionally replaced with —O—;
(iii) 1 or 2 non-terminal carbons of the C₂₋₁₀ alkyl are optionally replaced with —NR¹⁰—;
(iv) 1 or 2 non-terminal carbons of the C₂₋₁₀ alkyl are optionally replaced with —C(═O)—; and
(v) 1 or 2 non-terminal carbons of the C₂₋₁₀ alkyl are optionally replaced with —CR^{a}R^{b}— wherein R^{a} and R^{b} together with the C atom to which they are attached form a C₃₋₆ cycloalkyl group.

In some embodiments, R₂ and R₃ are each independently selected from C₂₋₁₀ alkyl, wherein:
(i) the C₂₋₁₀ alkyl is substituted by 1, 2, 3, or 4 substituents independently selected from —NR⁸R⁹, —OH, and halo, wherein at least one substituent is —NR⁸R⁹; and
(ii) 1 or 2 non-terminal carbons of the C₂₋₁₀ alkyl are optionally replaced with —O—.

In some embodiments, R₂ and R₃ are each independently selected from C₂₋₁₀ alkyl, wherein:
(i) the C₂₋₁₀ alkyl is substituted by 1, 2, 3, or 4 substituents independently selected from —NR⁸R⁹, —OH, and halo, wherein at least one substituent is —NR⁸R⁹; and
(iii) 1 or 2 non-terminal carbons of the C₂₋₁₀ alkyl are optionally replaced with —NR¹⁰—.

In some embodiments, R₂ and R₃ are each independently selected from C₂₋₁₀ alkyl, wherein:
(i) the C₂₋₁₀ alkyl is substituted by 1, 2, 3, or 4 substituents independently selected from —NR⁸R⁹, —OH, and halo, wherein at least one substituent is —NR⁸R⁹; and
(iv) 1 or 2 non-terminal carbons of the C₂₋₁₀ alkyl are optionally replaced with —C(═O)—.

In some embodiments, R₂ and R₃ are each independently selected from C₂₋₁₀ alkyl, wherein:
(i) the C₂₋₂₀ alkyl is substituted by 1, 2, 3, or 4 substituents independently selected from —NR⁸R⁹, —OH, and halo, wherein at least one substituent is —NR⁸R⁹; and
(v) 1 or 2 non-terminal carbons of the C₂₋₁₀ alkyl are optionally replaced with —CR^{a}R^{b}— wherein R^{a} and R^{b} together with the C atom to which they are attached form a C₃₋₆ cycloalkyl group.

In some embodiments, R₂ and R₃ are each independently selected from C₂₋₁₀ alkyl, wherein:
(i) the C₂₋₁₀ alkyl is substituted by 1, 2, 3, or 4 substituents independently selected from —NR⁸R⁹, —OH, and halo, wherein at least one substituent is —NR⁸R⁹;
(ii) 1 or 2 non-terminal carbons of the C₂₋₁₀ alkyl are optionally replaced with —O—; and
(iii) 1 or 2 non-terminal carbons of the C₂₋₁₀ alkyl are optionally replaced with —NR¹⁰—.

In some embodiments, R² and R³ are each independently selected from C₄₋₁₀ alkyl, wherein:
(i) the C₄₋₁₀ alkyl is substituted by 1, 2, 3, or 4 substituents independently selected from —NR⁸R⁹, —OH, and halo, wherein at least one substituent is —NR⁸R⁹;
(ii) 1 or 2 non-terminal carbons of the C₄₋₁₀ to alkyl are optionally replaced with —O—;
(iii) 1 or 2 non-terminal carbons of the C₄₋₁₀ alkyl are optionally replaced with —NR¹⁰—;
(iv) 1 or 2 non-terminal carbons of the C₄₋₁₀ alkyl are optionally replaced with —C(═O)—; and
(v) 1 or 2 non-terminal carbons of the C₄₋₁₀ alkyl are optionally replaced with —CR^{a}R^{b}— wherein R^{a} and R^{b} together with the C atom to which they are attached form a C₃₋₆ cycloalkyl group.

In some embodiments, R² and R³ are each independently selected from C₄₋₁₀ alkyl, wherein:
(i) the C₄₋₁₀ alkyl is substituted by 1, 2, 3, or 4 substituents independently selected from —NR⁸R⁹, —OH, and halo, wherein at least one substituent is —NR⁸R⁹;
(ii) 1 or 2 non-terminal carbons of the C₄₋₁₀ alkyl are optionally replaced with —O—; and
(iii) 1 or 2 non-terminal carbons of the C₄₋₁₀ alkyl are optionally replaced with —NR¹⁰—.

In some embodiments, one of R² and R³ is C₂₋₅ alkyl, wherein:
the C₂₋₅ alkyl is substituted by 1, 2, 3, 4, or 5 substituents independently selected from —NR⁸R⁹, —OH, and halo, wherein at least one substituent is —NR⁸R⁹;
and wherein the other of R₂ and R₃ is C₇₋₁₀ alkyl, wherein:
(i) the C₇₋₁₀ alkyl is substituted by 1, 2, 3, 4, or 5 substituents independently selected from —NR⁸R⁹, —OH, and halo, wherein at least one substituent is —NR⁸R⁹;
(ii) 1, 2, 3, or 4 non-terminal carbons of the C₇₋₁₀ alkyl are optionally replaced with —O—;
(iii) 1, 2, 3, or 4 non-terminal carbons of the C₇₋₁₀ alkyl are optionally replaced with —NR¹⁰—;
(iv) 1, 2, 3, or 4 non-terminal carbons of the C₇₋₁₀ alkyl are optionally replaced with —C(═O)—; and
(v) 1, 2, 3, or 4 non-terminal carbons of the C₇₋₁₀ alkyl are optionally replaced with —CR^{a}R^{b}— wherein R^{a} and R^{b} together with the C atom to which they are attached form a C₃₋₆ cycloalkyl group.

In some embodiments, one of R₂ and R₃ is C₂₋₅ alkyl, wherein:
(i) the C₂₋₅ alkyl is substituted by 1, 2, 3, 4, or 5 substituents independently selected from —NR⁸R⁹, —OH, and halo, wherein at least one substituent is —NR⁸R⁹;
and wherein the other of R₂ and R₃ is C₇₋₁₀ alkyl, wherein:
(i) the C₇₋₁₀ alkyl is substituted by 1, 2, 3, 4, or 5 substituents independently selected from —NR⁸R⁹, —OH, and halo, wherein at least one substituent is —NR⁸R⁹;
(ii) 1, 2, 3, or 4 non-terminal carbons of the C₇₋₁₀ alkyl are optionally replaced with —O—; and
(iii) 1, 2, 3, or 4 non-terminal carbons of the C₇₋₁₀ alkyl are optionally replaced with —NR¹⁰—.

In some embodiments, one of R² and R³ is C₂₋₂₀ alkyl substituted by 1-NR⁸R⁹. In some embodiments, one of R² and R³ is C₂₋₂₀ alkyl substituted by 1-NR⁸R⁹ and 1 non-terminal carbon of the C₂₋₂₀ alkyl is replaced with —NR¹⁰—. In some embodiments, one of R² and R³ is C₂₋₂₀ alkyl substituted by 1-NR⁸R⁹ and 1 non-terminal carbon of the C₂₋₂₀ alkyl is replaced with —O—. In some embodiments, one of R² and R³ is C₂₋₂₀ alkyl substituted by 1-NR⁸R⁹ and 2 halo and 1 non-terminal carbon of the C₂₋₂₀ alkyl is replaced with —NR¹⁰—. In some embodiments, one of R² and R³ is C₂₋₂₀ alkyl substituted by 1-NR⁸R⁹ and 2-F and 1 non-terminal carbon of the C₂₋₂₀ alkyl is replaced with —NR¹⁰—. In some embodiments, one of R² and R³ is C₂₋₂₀ alkyl substituted by 1-NR⁸R⁹ and 2 halo. In some embodiments, one of R² and R³ is C₂₋₂₀ alkyl substituted by 1-NR⁸R⁹ and 2-F. In some embodiments, one of R² and R³ is C₂₋₂₀ alkyl substituted by 1-NR⁸R⁹ and 1 halo, wherein 1 non-terminal carbon of the C₂₋₂₀ alkyl is replaced with —NR¹⁰—. In some embodiments, one of R² and R³ is C₂₋₂₀ alkyl substituted by 1-NR⁸R⁹ and 1-F, wherein 1 non-terminal carbon of the C₂₋₂₀ alkyl is replaced with —NR¹⁰—. In some embodiments, one of R² and R³ is C₂₋₂₀ alkyl substituted by 1-NR⁸R⁹ and 1 halo. In some embodiments, one of R² and R³ is C₂₋₂₀ alkyl substituted by 1-NR⁸R⁹ and 1-F. In some embodiments, one of R² and R³ is C₂₋₂₀ alkyl substituted by 1-NR⁸R⁹ and 1-OH.

In some embodiments, one of R² and R³ is C₂₋₂₀ alkyl substituted by 1-NR⁸R⁹ and 1 non-terminal carbon of the C₂₋₂₀ alkyl is replaced with —C(═O)—. In some embodiments, one of R² and R³ is C₂₋₂₀ alkyl substituted by 2-NR⁸R⁹ and 1 non-terminal carbon of the C₂₋₂₀ alkyl is replaced with —C(═O)—. In some embodiments, one of R² and R³ is C₂₋₂₀ alkyl substituted by 1-NR⁸R⁹, 1 non-terminal carbon of the C₂₋₂₀ alkyl is replaced with —NR¹⁰—, and 1 non-terminal carbon of the C₂₋₂₀ alkyl is replaced with —CR^{a}R^{b}— wherein R^{a} and R^{b} together with the C atom to which they are attached form a C₃₋₆ cycloalkyl group. In some embodiments, one of R² and R³ is C₂₋₂₀ alkyl substituted by 1-NR⁸R⁹ and 1 non-terminal carbon of the C₂₋₂₀ alkyl is replaced with —CR^{a}R^{b}— wherein R^{a} and R^{b} together with the C atom to which they are attached form a C₃₋₆ cycloalkyl group.

In some embodiments, one of R² and R³ is selected from:
C₂₋₂₀ alkyl substituted by 1-NR⁸R⁹,
C₂₋₂₀ alkyl substituted by 1-NR⁸R⁹ wherein 1 non-terminal carbon of the C₂₋₂₀ alkyl is replaced with —NR¹⁰—,
C₂₋₂₀ alkyl substituted by 1-NR⁸R⁹ wherein 1 non-terminal carbon of the C₂₋₂₀ alkyl is replaced with —O—,
C₂₋₂₀ alkyl substituted by 1-NR⁸R⁹ and 2 halo wherein 1 non-terminal carbon of the C₂₋₂₀ alkyl is replaced with —NR¹⁰—,
C₂₋₂₀ alkyl substituted by 1-NR⁸R⁹ and 1 halo wherein 1 non-terminal carbon of the C₂₋₂₀ alkyl is replaced with —NR¹⁰—,
C₂₋₂₀ alkyl substituted by 1-NR⁸R⁹ wherein 1 non-terminal carbon of the C₂₋₂₀ alkyl is replaced with —C(═O)—,
C₂₋₂₀ alkyl substituted by 2-NR⁸R⁹ wherein 1 non-terminal carbon of the C₂₋₂₀ alkyl is replaced with —C(═O)—, and
C₂₋₂₀ alkyl substituted by 1-NR⁸R⁹ wherein 1 non-terminal carbon of the C₂₋₂₀ alkyl is replaced with —NR¹⁰— and 1 non-terminal carbon of the C₂₋₂₀ alkyl is replaced with —CR^{a}R^{b}— wherein R^{a} and R^{b} together with the C atom to which they are attached form a C₃₋₆ cycloalkyl group, and
the other of R² and R³ is selected from:
C₂₋₂₀ alkyl substituted by 1-NR⁸R⁹,
C₂₋₂₀ alkyl substituted by 1-NR⁸R⁹ wherein 1 non-terminal carbon of the C₂₋₂₀ alkyl is replaced with —NR¹⁰—,
C₂₋₂₀ alkyl substituted by 1-NR⁸R⁹ and 2 halo,
C₂₋₂₀ alkyl substituted by 1-NR⁸R⁹ and 1 halo,
C₂₋₂₀ alkyl substituted by 1-NR⁸R⁹ and 1-OH, and
C₂₋₂₀ alkyl substituted by 1-NR⁸R⁹ wherein 1 non-terminal carbon of the C₂₋₂₀ alkyl is replaced with CR^{a}R^{b} wherein R^{a} and R^{b} together with the C atom to which they are attached form a C₃₋₆ cycloalkyl group.

In some embodiments, one of R² and R³ is selected from C₂₋₂₀ alkyl substituted by 1-NR⁸R⁹, C₂₋₂₀ alkyl substituted by 1-NR⁸R⁹ wherein 1 non-terminal carbon of the C₂₋₂₀ alkyl is replaced with —NR¹⁰—, C₂₋₂₀ alkyl substituted by 1-NR⁸R⁹ wherein 1 non-terminal carbon of the C₂₋₂₀ alkyl is replaced with 0, C₂₋₂₀ alkyl substituted by 1-NR⁸R⁹ and 2 halo wherein 1 non-terminal carbon of the C₂₋₂₀ alkyl is replaced with —NR¹⁰—, and C₂₋₂₀ alkyl substituted by 1-NR⁸R⁹ and 1 halo wherein 1 non-terminal carbon of the C₂₋₂₀ alkyl is replaced with —NR¹⁰—, and the other of R₂ and R₃ is selected from C₂₋₂₀ alkyl substituted by 1-NR⁸R⁹, C₂₋₂₀ alkyl substituted by 1-NR⁸R⁹ wherein 1 non-terminal carbon of the C₂₋₂₀ alkyl is replaced with —NR¹⁰—, C₂₋₂₀ alkyl substituted by 1-NR⁸R⁹ and 2 halo, C₂₋₂₀ alkyl substituted by 1-NR⁸R⁹ and 1 halo, and C₂₋₂₀ alkyl substituted by 1-NR⁸R⁹ and 1-OH.

In some embodiments, one of R² and R³ is C₂₋₁₀ alkyl substituted by 1-NR⁸R⁹. In some embodiments, one of R² and R³ is C₂₋₁₀ alkyl substituted by 1-NR⁸R⁹ and 1 non-terminal carbon of the C₂₋₁₀ alkyl is replaced with —NR¹⁰—. In some embodiments, one of R² and R³ is C₂₋₁₀ alkyl substituted by 1-NR⁸R⁹ and 1 non-terminal carbon of the C₂₋₁₀ alkyl is replaced with —O—. In some embodiments, one of R² and R³ is C₂ alkyl substituted by 1-NR⁸R⁹ and 2 halo and 1 non-terminal carbon of the C₂₋₁₀ alkyl is replaced with —NR¹⁰—. In some embodiments, one of R² and R³ is C₂₋₁₀ alkyl substituted by 1-NR⁸R⁹ and 2-F and 1 non-terminal carbon of the C₂₋₁₀ alkyl is replaced with —NR¹⁰—. In some embodiments, one of R² and R³ is C₂₋₁₀ alkyl substituted by 1-NR⁸R⁹ and 2 halo. In some embodiments, one of R² and R³ is C₂₋₁₀ alkyl substituted by 1-NR⁸R⁹ and 2-F. In some embodiments, one of R² and R³ is C₂₋₁₀ alkyl substituted by 1-NR⁸R⁹ and 1 halo wherein 1 non-terminal carbon of the C₂₋₁₀ alkyl is replaced with —NR¹⁰—. In some embodiments, one of R² and R³ is C₂₋₁₀ alkyl substituted by 1-NR⁸R⁹ and 1-F wherein 1 non-terminal carbon of the C₂₋₁₀ alkyl is replaced with —NR—. In some embodiments, one of R² and R³ is C₂₋₁₀ alkyl substituted by 1-NR⁸R⁹ and 1 halo. In some embodiments, one of R² and R³ is C₂₋₁₀ alkyl substituted by 1-NR⁸R⁹ and 1-F. In some embodiments, one of R² and R³ is C₂₋₁₀ alkyl substituted by 1-NR⁸R⁹ and 1-OH.

In some embodiments, one of R² and R³ is C₂₋₁₀ alkyl substituted by 1-NR⁸R⁹ and 1 non-terminal carbon of the C₂₋₁₀ alkyl is replaced with —C(═O)—. In some embodiments, one of R² and R³ is C₂₋₁₀ alkyl substituted by 2-NR⁸R⁹ and 1 non-terminal carbon of the C₂₋₁₀ alkyl is replaced with —C(═O)—. In some embodiments, one of R² and R³ is C₂₋₁₀ alkyl substituted by 1-NR⁸R⁹, 1 non-terminal carbon of the C₂₋₁₀ alkyl is replaced with —NR⁸R⁹ and 1 non-terminal carbon of the C₂₋₁₀ alkyl is replaced with CR^{a}R^{b} wherein R^{a} and R^{b} together with the C atom to which they are attached form a C₃₋₆ cycloalkyl group. In some embodiments, one of R² and R³ is C₂₋₁₀ alkyl substituted by 1-NR⁸R⁹ and 1 non-terminal carbon of the C₂ alkyl is replaced with —CR^{a}R^{b}— wherein R^{a} and R^{b} together with the C atom to which they are attached form a C₃₋₆ cycloalkyl group.

In some embodiments, one of R² and R³ is selected from:
C₂₋₁₀ alkyl substituted by 1-NR⁸R⁹,
C₂₋₁₀ alkyl substituted by 1-NR⁸R⁹ wherein 1 non-terminal carbon of the C₂₋₁₀ alkyl is replaced with —NR¹⁰—,
C₂₋₁₀ alkyl substituted by 1-NR⁸R⁹ wherein 1 non-terminal carbon of the C₂₋₁₀ alkyl is replaced with —O—,
C₂₋₁₀ alkyl substituted by 1-NR⁸R⁹ and 2 halo wherein 1 non-terminal carbon of the C₂₋₁₀ alkyl is replaced with —NR¹⁰—,
C₂₋₁₀ alkyl substituted by 1-NR⁸R⁹ and 1 halo wherein 1 non-terminal carbon of the C₂₋₁₀ alkyl is replaced with —NR¹⁰—,
C₂₋₁₀ alkyl substituted by 1-NR⁸R⁹ wherein 1 non-terminal carbon of the C₂₋₁₀ alkyl is replaced with —C(═O)—,
C₂₋₁₀ alkyl substituted by 2-NR⁸R⁹ wherein 1 non-terminal carbon of the C₂₋₁₀ alkyl is replaced with —C(═O)—, and
C₂₋₁₀ alkyl substituted by 1-NR⁸R⁹ wherein 1 non-terminal carbon of the C₂₋₁₀ alkyl is replaced with NR¹⁰ and 1 non-terminal carbon of the C₂₋₁₀ alkyl is replaced with —CR^{a}R^{b}— wherein R^{a} and R^{b} together with the C atom to which they are attached form a C₃₋₆ cycloalkyl group, and
the other of R² and R³ is selected from:
C₂₋₁₀ alkyl substituted by 1-NR⁸R⁹,
C₂₋₁₀ alkyl substituted by 1-NR⁸R⁹ wherein 1 non-terminal carbon of the C₂₋₁₀ alkyl is replaced with —NR¹⁰—,
C₂₋₁₀ alkyl substituted by 1-NR⁸R⁹ and 2 halo,
C₂₋₁₀ alkyl substituted by 1-NR⁸R⁹ and 1 halo,
C₂₋₁₀ alkyl substituted by 1-NR⁸R⁹ and 1-OH, and
C₂₋₁₀ alkyl substituted by 1-NR⁸R⁹ wherein 1 non-terminal carbon of the C₂₋₁₀ alkyl is replaced with —CR^{a}R^{b}— wherein R^{a} and R^{b} together with the C atom to which they are attached form a C₃₋₆ cycloalkyl group.

In some embodiments, one of R² and R³ is selected from C₂₋₁₆ alkyl substituted by 1-NR⁸R⁹, C₂₋₁₀ alkyl substituted by 1-NR⁸R⁹ wherein 1 non-terminal carbon of the C₂₋₁₀ alkyl is replaced with —NR¹⁰—, C₂₋₁₀ alkyl substituted by 1-NR⁸R⁹ wherein 1 non-terminal carbon of the C₂₋₁₀ alkyl is replaced with —O—, C₂₋₁₀ alkyl substituted by 1-NR⁸R⁹ and 2 halo wherein 1 non-terminal carbon of the C₂₋₁₀ alkyl is replaced with —NR¹⁰—, and C₂₋₁₀ alkyl substituted by 1-NR⁸R⁹ and 1 halo wherein 1 non-terminal carbon of the C₂₋₁₀ alkyl is replaced with —NR¹⁰—, and the other of R² and R³ is selected from C₂₋₁₀ alkyl substituted by 1-NR⁸R⁹, C₂₋₁₀ alkyl substituted by 1-NR⁸R⁹ wherein 1 non-terminal carbon of the C₂₋₁₀ alkyl is replaced with —NR¹⁰—, C₂₋₁₀ alkyl substituted by 1-NR⁸R⁹ and 2 halo, C₂₋₁₀ alkyl substituted by 1-NR⁸R⁹ and 1 halo, and C₂₋₁₀ alkyl substituted by 1-NR⁸R⁹ and 1-OH.

In some embodiments, one of R² and R³ is selected from:
C₅₋₁₀ alkyl substituted by 1-NR⁸R⁹,
C₅₋₁₀ alkyl substituted by 1-NR⁸R⁹ wherein 1 non-terminal carbon of the C₅₋₁₀ alkyl is replaced with —NR¹⁰—,
C₅₋₁₀ alkyl substituted by 1-NR⁸R⁹ wherein 1 non-terminal carbon of the CS₁₀ alkyl is replaced with —O—,
C₅₋₁₀ alkyl substituted by 1-NR⁸R⁹ and 2 halo wherein 1 non-terminal carbon of the C₅₋₁₀ alkyl is replaced with —NR¹⁰—,
C₅₋₁₀ alkyl substituted by 1-NR⁸R⁹ and 1 halo wherein 1 non-terminal carbon of the C₅₋₁₀ alkyl is replaced with —NR¹⁰—, and
C₁₀ alkyl substituted by 1-NR⁸R⁹ wherein 1 non-terminal carbon of the C₂₋₁₀ alkyl is replaced with —NR¹⁰— and 1 non-terminal carbon of the C₂₋₁₀ alkyl is replaced with —CR^{a}R^{b}— wherein R^{a} and R^{b} together with the C atom to which they are attached form a C₃₋₆ cycloalkyl group,
and the other of R² and R³ is selected from:
C₃₋₆ alkyl substituted by 1-NR⁸R⁹,
C₃₋₆ alkyl substituted by 1-NR⁸R⁹ wherein 1 non-terminal carbon of the C₃₋₆ alkyl is replaced with —NR¹⁰—,
C₃₋₆ alkyl substituted by 1-NR⁸R⁹ and 2 halo,
C₃₋₆ alkyl substituted by 1-NR⁸R⁹ and 1 halo,
C₃₋₆ alkyl substituted by 1-NR⁸R⁹ and 1-OH,
C₃₋₆ alkyl substituted by 1-NR⁸R⁹ wherein 1 non-terminal carbon of the C₂₋₁₀ alkyl is replaced with —C(═O)—,
C₃₋₆ alkyl substituted by 2-NR⁸R⁹ wherein 1 non-terminal carbon of the C₂₋₁₀ alkyl is replaced with —C(═O)—, and
C₃₋₆ alkyl substituted by 1-NR⁸R⁹ wherein 1 non-terminal carbon of the C₂₋₁₀ alkyl is replaced with —CR^{a}R^{b}— wherein R^{a} and R^{b} together with the C atom to which they are attached form a C₃₋₆ cycloalkyl group.

In some embodiments, one of R² and R³ is selected from C₅₋₁₀ alkyl substituted by 1-NR⁸R⁹, C₅₋₁₀ alkyl substituted by 1-NR⁸R⁹ wherein 1 non-terminal carbon of the C₅₋₁₀ alkyl is replaced with —NR¹⁰—, C₅₋₁₀ alkyl substituted by 1-NR⁸R⁹ wherein 1 non-terminal carbon of the C₅₋₁₀ alkyl is replaced with —O—, C₅₋₁₀ alkyl substituted by 1-NR⁸R⁹ and 2 halo wherein 1 non-terminal carbon of the C₅₋₁₀ alkyl is replaced with —NR¹⁰—, and C₅₋₁₀ alkyl substituted by 1-NR⁸R⁹ and 1 halo wherein 1 non-terminal carbon of the C₅₋₁₀ alkyl is replaced with —NR¹⁰—, and the other of R² and R³ is selected from C₃₋₆ alkyl substituted by 1-NR⁸R⁹, C₃₋₆ alkyl substituted by 1-NR⁸R⁹ wherein 1 non-terminal carbon of the C₃₋₆ alkyl is replaced with —NR¹⁰—, C₃₋₆ alkyl substituted by 1-NR⁸R⁹ and 2 halo, C₃₋₆ alkyl substituted by 1-NR⁸R⁹ and 1 halo, and C₃₋₆ alkyl substituted by 1-NR⁸R⁹ and 1-OH.

In some embodiments, one of R² and R³ is C₃ alkyl which is substituted by at least one —NR⁸R⁹ group and is further optionally substituted by one or two groups selected from —OH and halo.

In some embodiments, one of R² and R³ is selected from

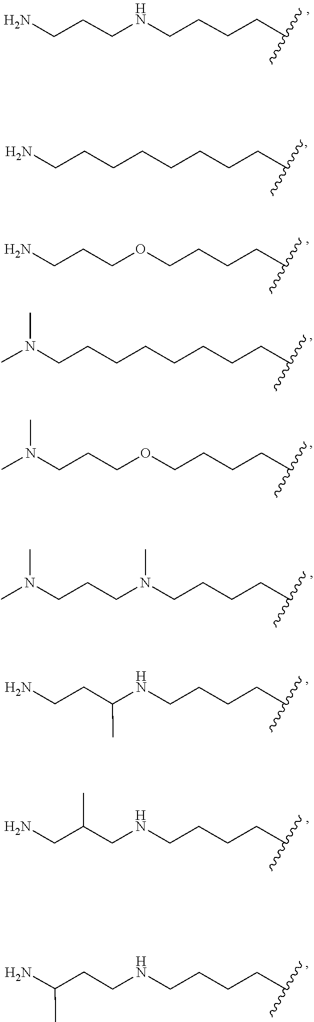

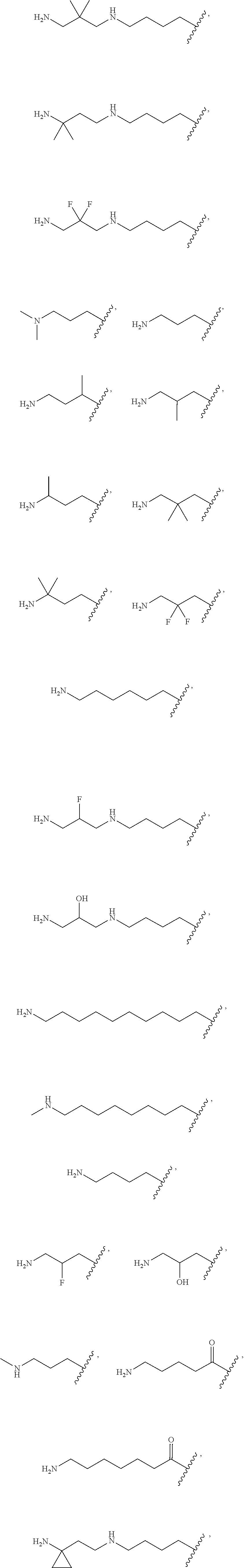

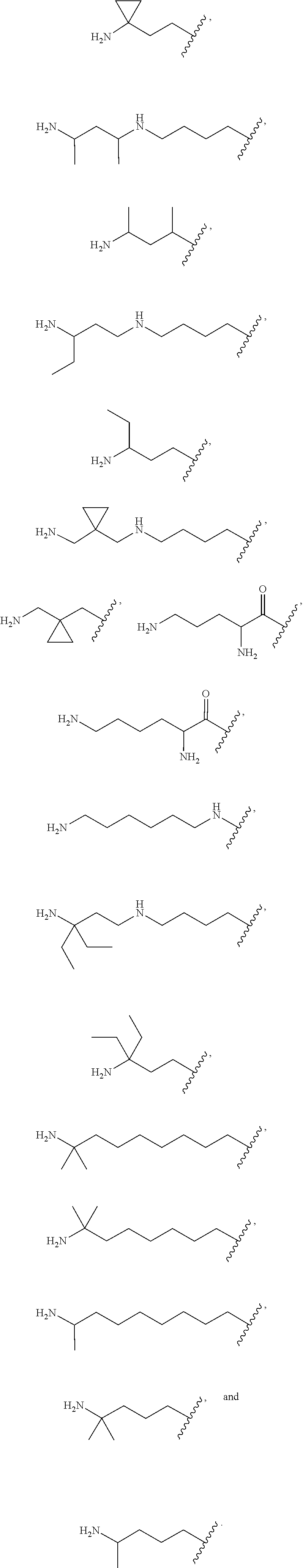

In some embodiments, one of R² and R³ is selected from

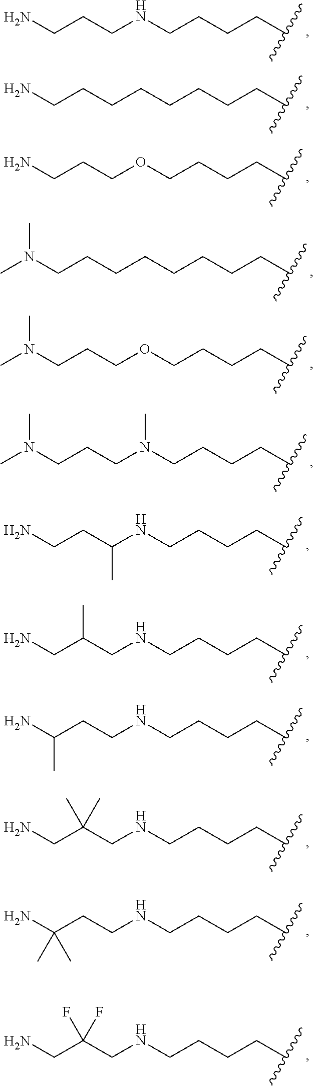

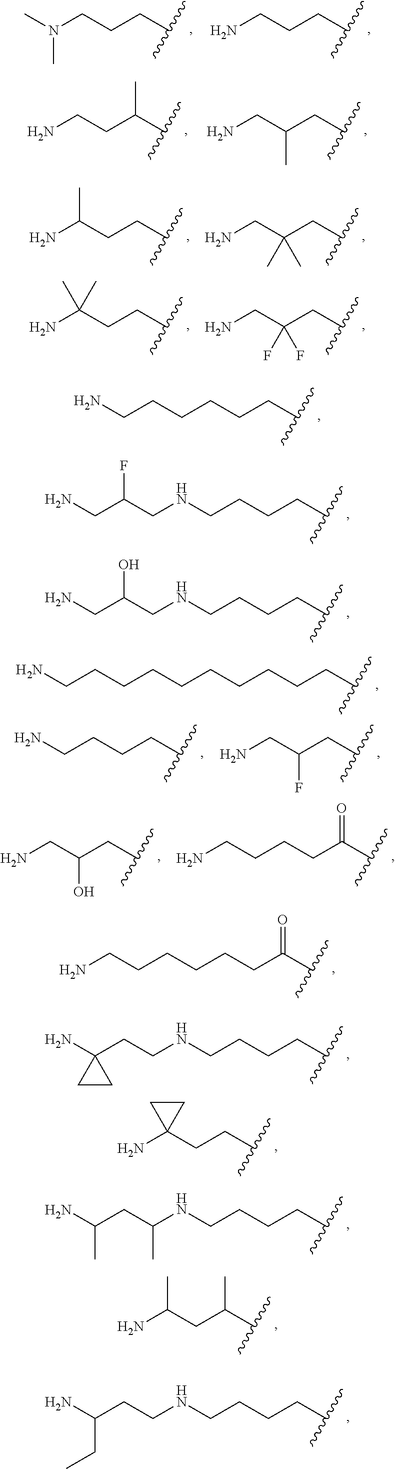

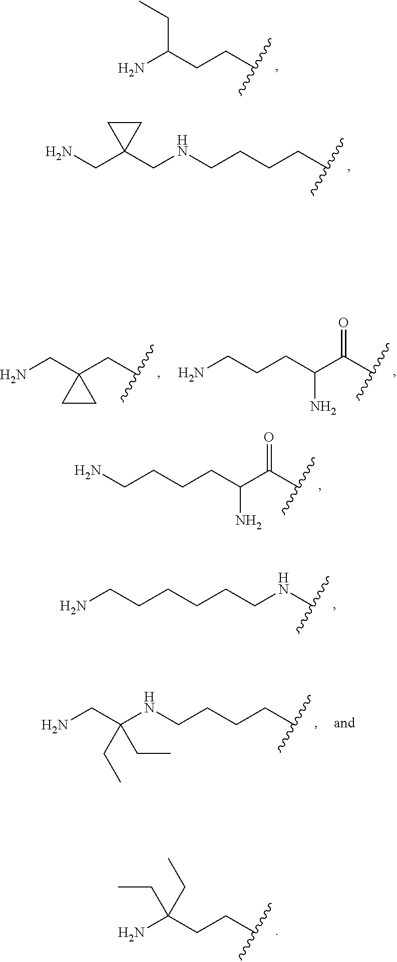

In some embodiments, one of R² and R³ is selected from

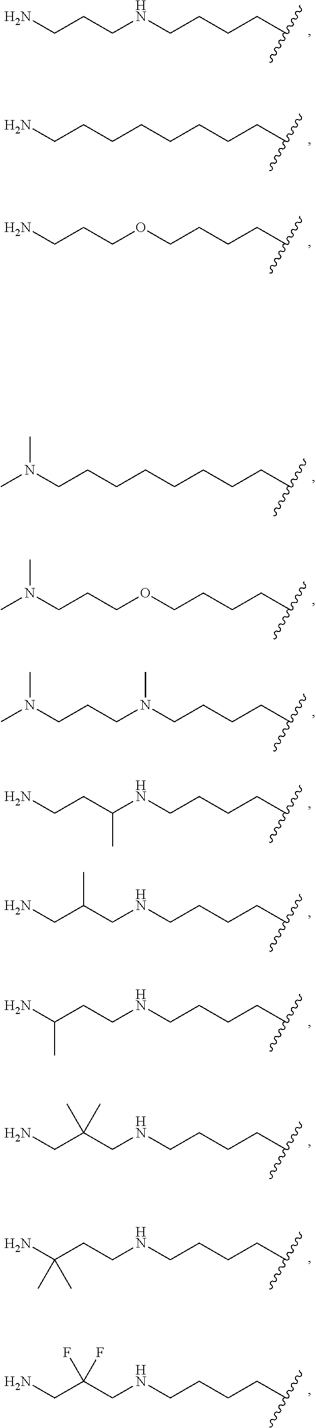

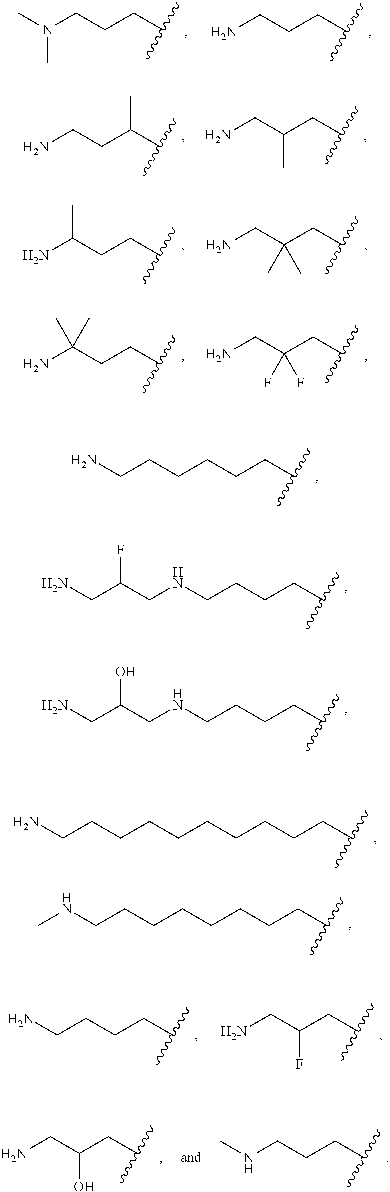

In some embodiments, one of R² and R³ is selected from

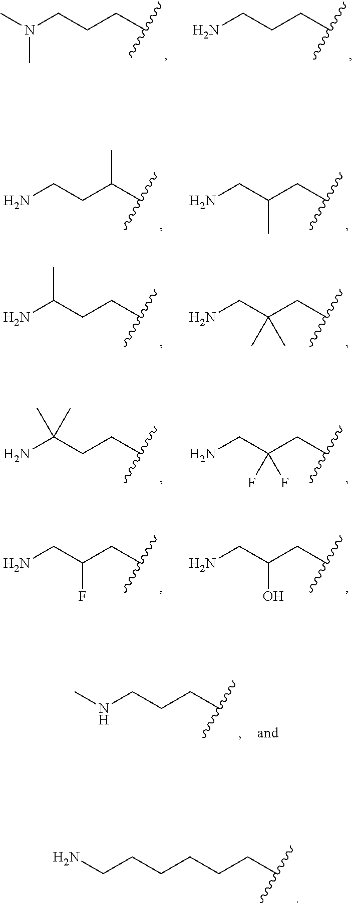

In some embodiments, one of R² and R³ is selected from

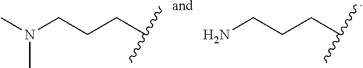

In some embodiments, one of R² and R³ is selected from

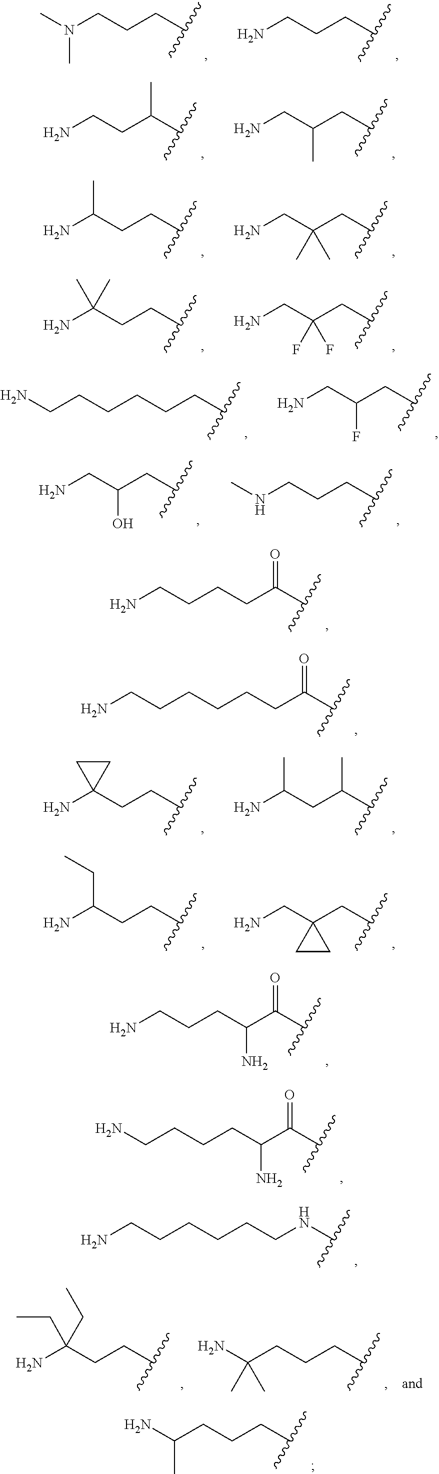

and the other of R² and R³ is selected from

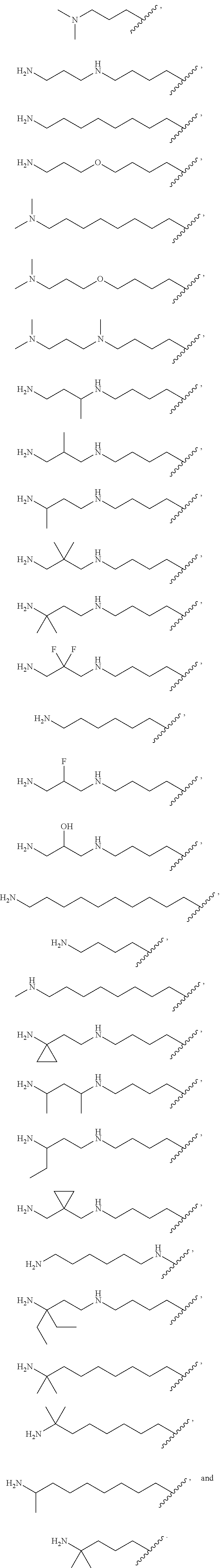

In some embodiments, one of R² and R³ is selected from

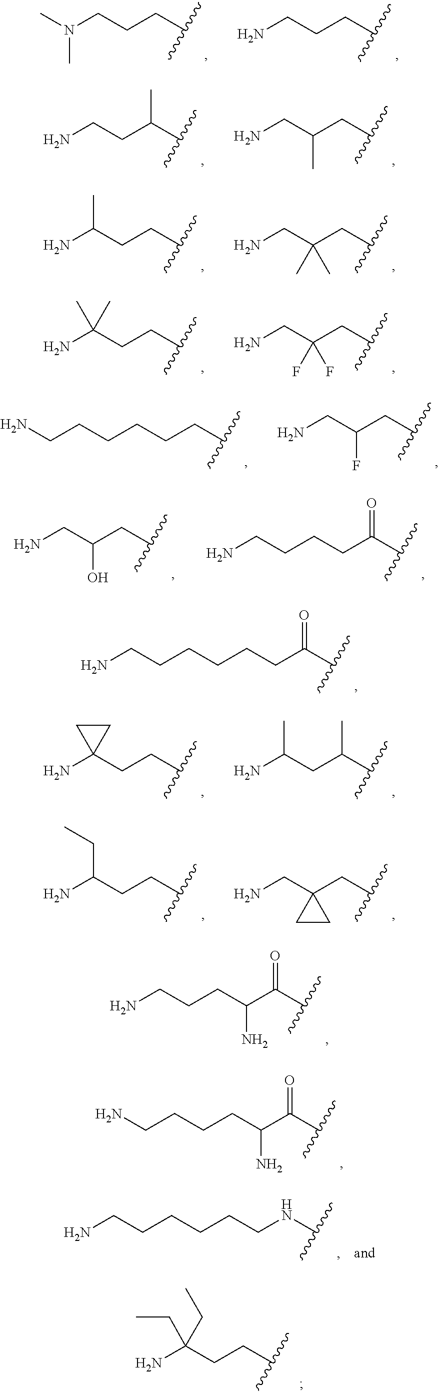

and the other of R² and R³ is selected from

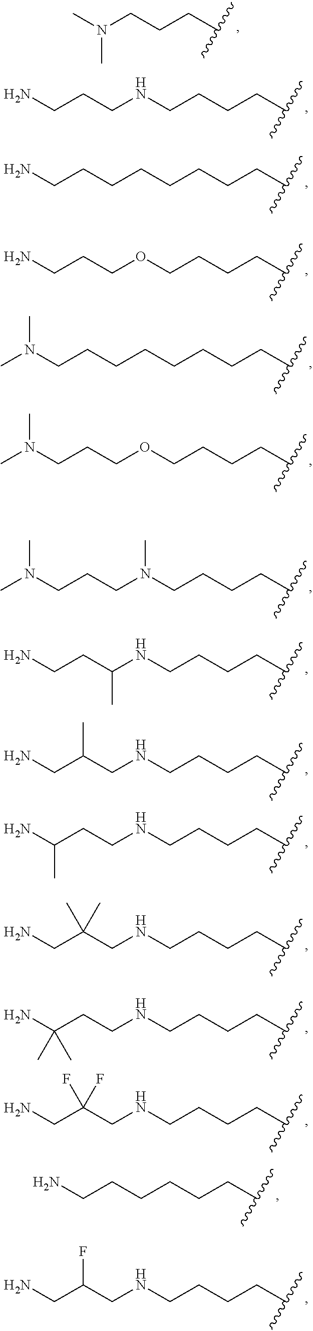

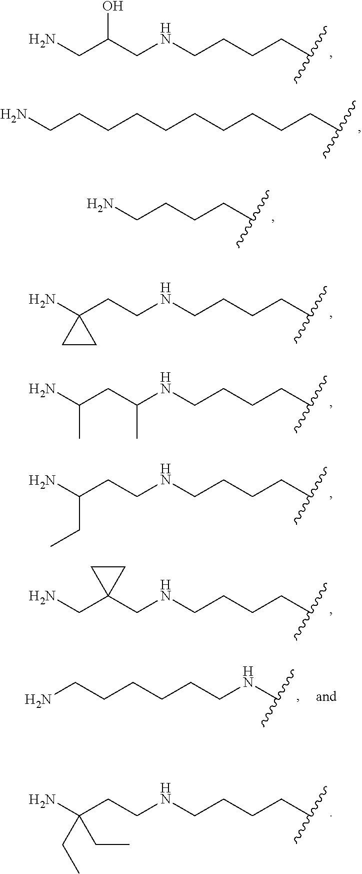

In some embodiments, one of R² and R³ is selected from

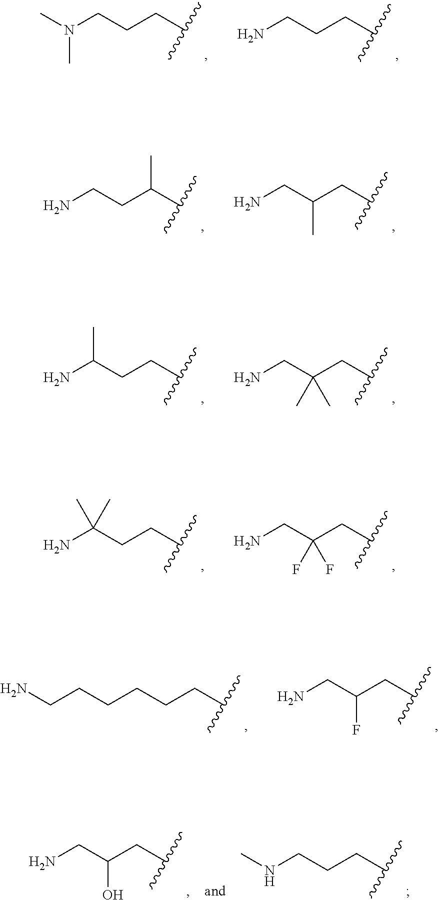

and the other of R² and R³ is selected from

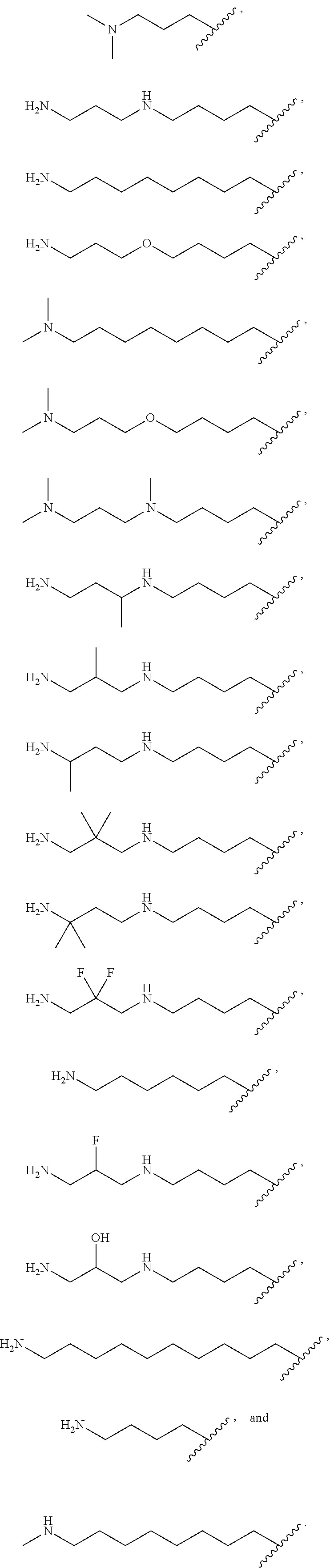

In some embodiments, R² and R³ together with the N atom to which they are attached form a 7-18 membered heterocycloalkyl group comprising 1, 2, or 3 ring-forming —NR¹⁰— groups, wherein the 7-18 membered heterocycloalkyl group is optionally substituted with 1, 2, or 3 substituents independently selected from C₁₋₄ alkyl, —NR⁸R⁹, —OH, and halo.

In some embodiments, R² and R³ together with the N atom to which they are attached form a 7-12 membered heterocycloalkyl group comprising 1, 2, or 3 ring-forming —NR¹⁰— groups, wherein the 7-12 membered heterocycloalkyl group is optionally substituted with 1, 2, or 3 substituents independently selected from C₁₋₄ alkyl, —NR⁸R⁹, —OH, and halo.

In some embodiments, R² and R³ together with the N atom to which they are attached form a 8-10 membered heterocycloalkyl group comprising 1, 2, or 3 ring-forming —NR¹⁰— groups, wherein the 8-10 membered heterocycloalkyl group is optionally substituted with 1, 2, or 3 substituents independently selected from C₁₋₄ alkyl, —NR⁸R⁹, —OH, and halo.

In some embodiments, R² and R³ together with the N atom to which they are attached form a 8-10 membered heterocycloalkyl group comprising 1, 2, or 3 ring-forming —NCH₃— or —NH— groups, wherein the 8-10 membered heterocycloalkyl group is optionally substituted with 1, 2, or 3 substituents independently selected from C₁₋₄ alkyl, —NR⁸R⁹, —OH, and halo.

In some embodiments, R² and R³ together with the N atom to which they are attached form an 8-10 membered heterocycloalkyl group comprising 1, 2, or 3 ring-forming —NCH₃— or —NH— groups.

In some embodiments, R² and R³ together with the N atom to which they are attached form a heterocycloalkyl group of formula:

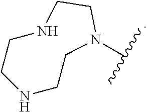 [SMILES: *N1CCNCCNCC1 ; "."]

In some embodiments, R², R³, and R⁶, together with the atoms to which they are attached and any intervening atoms, form a 7-18 membered bridged heterocycloalkyl group optionally substituted with 1, 2, or 3 substituents independently selected from C₁₋₄ alkyl, —NR⁸R⁹, —OH, and halo.

In some embodiments, R², R³, and R⁶, together with the atoms to which they are attached and any intervening atoms, form a 7-13 membered bridged heterocycloalkyl group optionally substituted with 1, 2, or 3 substituents independently selected from C₁₋₄ alkyl, —NR₈R₉, —OH, and halo.

In some embodiments, R, R³, and R⁶, together with the atoms to which they are attached and any intervening atoms, form a 7-10 membered bridged heterocycloalkyl group optionally substituted with 1, 2, or 3 substituents independently selected from C₁ 4 alkyl, —NR₈R₉, —OH, and halo.

In some embodiments, R², R³, and R⁶, together with the atoms to which they are attached and any intervening atoms, form a 7-10 membered bridged heterocycloalkyl group.

In some embodiments, R², R³, and R⁶, together with the atoms to which they are attached and any intervening atoms, form a 7-18 membered bridged heterocycloalkyl group having the formula:

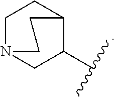 [SMILES: *C1CN2CCC1CC2 ; "."]

In some embodiments, R⁴ and R⁵ are each independently H or C₁₋₄ alkyl. In some embodiments, R⁴ and R⁵ are each independently H or methyl. In some embodiments, both R⁴ and R⁵ are H. In some embodiments, both R⁴ and R⁵ are C₁₋₄ alkyl. In some embodiments, both R⁴ and R⁵ are methyl. In some embodiments, one of R⁴ and R⁵ is H and the other of R⁴ and R⁵ is C₁₋₄ alkyl. In some embodiments, one of R⁴ and R⁵ is H and the other of R⁴ and R¹ is methyl.

In some embodiments, R⁶ and R⁷ are each independently H or C₁₋₄ alkyl. In some embodiments, R⁶ and R⁷ are each independently H or methyl. In some embodiments, both R⁶ and R⁷ are H. In some embodiments, both R⁶ and R⁷ are C₁₋₄ alkyl. In some embodiments, both R⁶ and R⁵ are methyl. In some embodiments, one of R⁶ and R⁷ is H and the other of R⁶ and R⁷ is C₁₋₄ alkyl. In some embodiments, one of R⁶ and R⁷ is H and the other of R⁶ and R⁷ is methyl.

In some embodiments, R⁸, R⁹, and R¹⁰ are each independently selected from H and methyl. In some embodiments, R⁸ and R⁹ are both H. In some embodiments, R⁸ and R⁹ are both C₁₋₄ alkyl. In some embodiments, R⁸ and R⁹ are both methyl. In some embodiments, one of R⁸ and R⁹ is H and the other of R⁸ and R⁹ is C₁₋₄ alkyl. In some embodiments, one of R⁸ and R⁹ is H and the other of R⁸ and R⁹ is methyl. In some embodiments, R¹⁰ is H or methyl. In some embodiments, R¹⁰ is H. In some embodiments, R¹⁰ is methyl.

In some embodiments, R^{a} and R^{b} together with the C atom to which they are attached form a C₃ cycloalkyl group such as cyclopropyl. In some embodiments, R^{a} and R^{b} together with the C atom to which they are attached form a C₄ cycloalkyl group such as cyclobutyl. In some embodiments, R^{a} and R^{b} together with the C atom to which they are attached form a C₅₋cycloalkyl group such as cyclopentyl. In some embodiments, R^{a} and R¹ together with the C atom to which they are attached form a C₆ cycloalkyl group such as cyclopentyl.

In some embodiments:
Z is N or CH;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1 or 2 substituents independently selected from —NR⁸R⁹, —OH, and halo, wherein at least one substituent is —NR⁸R⁹;
(ii) one non-terminal carbons of the C₂₋₂₀ alkyl are optionally replaced with —O—;
(iii) one non-terminal carbons of the C₂₋₂₀ alkyl are optionally replaced with —NR¹⁰—;
(iv) one non-terminal carbons of the C₂₋₂₀ alkyl are optionally replaced with —C(═O)—; and
(v) one non-terminal carbons of the C₂₋₂₀ alkyl are optionally replaced with —CR^{a}R^{b}— wherein R^{a} and R^{b} together with the C atom to which they are attached form a C₃₋₆ cycloalkyl group;
wherein R² and R³ are the same or different;
or R² and R³ together with the N atom to which they are attached form a 7-18 membered heterocycloalkyl group comprising 2 ring-forming —NR¹⁰— groups;
R⁴ is selected from H and C₁₋₄ alkyl;
R⁵, R⁶, and R⁷ are each H;
R⁸, R⁹, and R¹⁰ are each independently selected from H and C₁₋₄ alkyl;
j is 0 or 1;
k is 0, 1, 2, 3, or 4;
l is 0 or 1;
m is 0, 1, 2, or 4; and
n is 0 or 1;
wherein when j is 0, then l is 1,
wherein j and 1 are not both 0.

In some embodiments:
Z is N or CH;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1 or 2 substituents independently selected from —NR⁸R⁹, —OH, and halo, wherein at least one substituent is —NR⁸R⁹;
(ii) one non-terminal carbon of the C₂₋₂₀ alkyl are optionally replaced with —O—; and
(iii) one non-terminal carbon of the C₂₋₂₀ alkyl are optionally replaced with —NR¹⁰—;
wherein R² and R³ are the same or different;
or R² and R³ together with the N atom to which they are attached form a 7-18 membered heterocycloalkyl group comprising two ring-forming —NR¹⁰— groups;
R⁴ is selected from H and C₁₋₄ alkyl;
R⁵, R⁶, and R⁷ are each H;
R⁸, R⁹, and R¹⁰ are each independently selected from H and C₁₋₄ alkyl;
j is 0 or 1;
k is 0, 1, 2, 3, or 4;
l is 0 or 1;
m is 0, 1, 2, or 4; and
n is 0 or 1;
wherein j and 1 are not both 0,
wherein when j is 0, then l is 1.

In some embodiments,
Z is N;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1 or 2 substituents independently selected from —NR⁸R⁹, —OH, and halo, wherein at least one substituent is —NR⁸R⁹;
(ii) one non-terminal carbon of the C₂₋₂₀ alkyl is optionally replaced with —O—; and
(iii) one non-terminal carbon of the C₂₋₂₀ alkyl is optionally replaced with —NR¹⁰—;
wherein R² and R³ are the same or different;
or R² and R³ together with the N atom to which they are attached form a 7-18 membered heterocycloalkyl group comprising two ring-forming —NR¹⁰— groups;
R⁴ is selected from H and C₁₋₄ alkyl;
R⁵, R⁶, and R⁷ are each H;
R⁸, R⁹, and R¹⁰ are each independently selected from H and C₁₋₄ alkyl;
j is 0 or 1;
k is 0, 1, 2, 3, or 4;
l is 0 or 1;
m is 0, 1, or 4; and
n is 0 or 1;
wherein j and 1 are not both 0,
wherein when j is 0, then l is 1.

In some embodiments, the compound of Formula A6 is a compound of Formula A7:

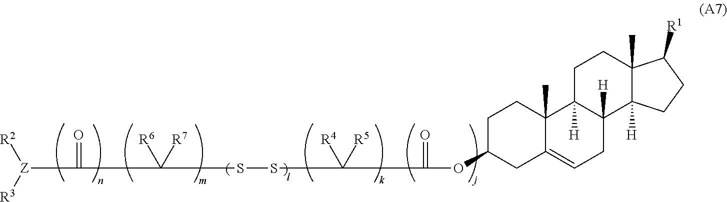

or a salt thereof.

In some embodiments, the sterol amine has Formula A8:

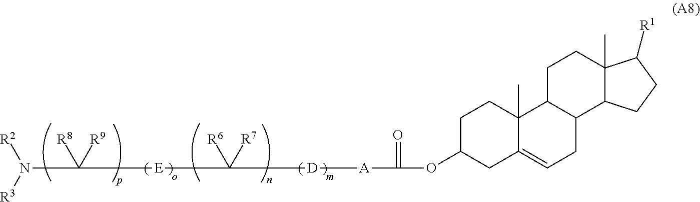

or a salt thereof, wherein:
A is —NR^{a}— or —CR⁴R⁵—;
D is —O— or —S—S—;
E is —C(O)—, —C(O)NH—, or —O—;
R¹ is C₁₋₁₄ alkyl, C₁₋₁₄ alkenyl, or C₁₋₁₄ hydroxyalkyl;
R² and R³ are each independently selected from H, methyl, and ethyl, wherein the methyl or ethyl is optionally substituted by —OH;
or R² and R³ together with the N atom to which they are attached form a 7-18 membered heterocycloalkyl group comprising 1, 2, or 3 ring-forming —NR¹⁰— groups, wherein the 7-18 membered heterocycloalkyl group is optionally substituted with 1, 2, or 3 substituents independently selected from C₁₋₄ alkyl, —NR₈R₉, —OH, and halo;
or R², R³, and R⁸, together with the atoms to which they are attached and any intervening atoms, form a 7-18 membered bridged heterocycloalkyl group optionally substituted with 1, 2, or 3 substituents independently selected from C₁₋₄ alkyl, —NR₈R₉, —OH, and halo;
R^{a} is H or methyl;
R⁴, R⁵, R⁶, R⁷, R⁸, R⁹, and R¹⁰ are each independently selected from H and C₁₋₄ alkyl;
or R⁴ and R⁵ together with the carbon atom to which they are attached form a C₃₋₅ cycloalkyl group;
or R⁶ and R⁷ together with the carbon atom to which they are attached form a C₃₋₅ cycloalkyl group;
or R⁸ and R⁹ together with the carbon atom to which they are attached form a C₃₋₅ cycloalkyl group;
m is 0 or 1;
n is 0, 1, 2, 3, 4, or 5;
o is 0 or 1; and
p is 0, 1, 2, 3, 4, 5, 6, 7, 8, 9, 10, 11, or 12;
wherein at least one of m, n, o, and p is other than 0;
wherein p is 1 when R², R³, and R⁸, together with the atoms to which they are attached and any intervening atoms, form a 7-18 membered bridged heterocycloalkyl group optionally substituted with 1, 2, or 3 substituents independently selected from C₁₋₄ alkyl, —NR₈R₉, —OH, and halo; and
wherein when m is 1, then A is —CR⁴R⁵— and n is 1.

In some embodiments, the compound is other than:

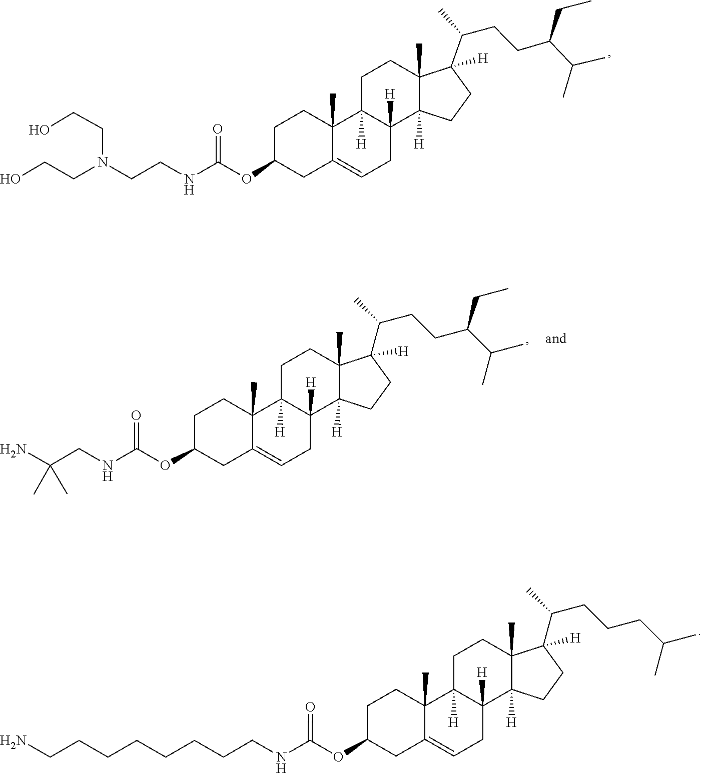

In some embodiments, the compound is other than:

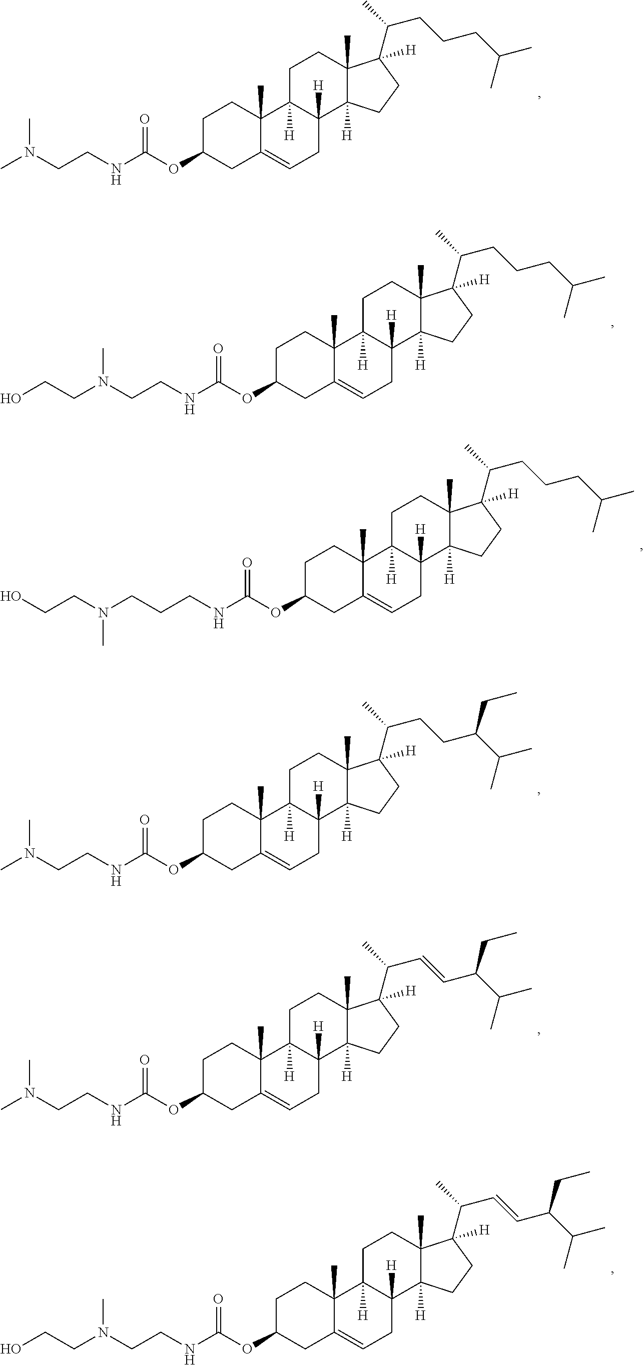

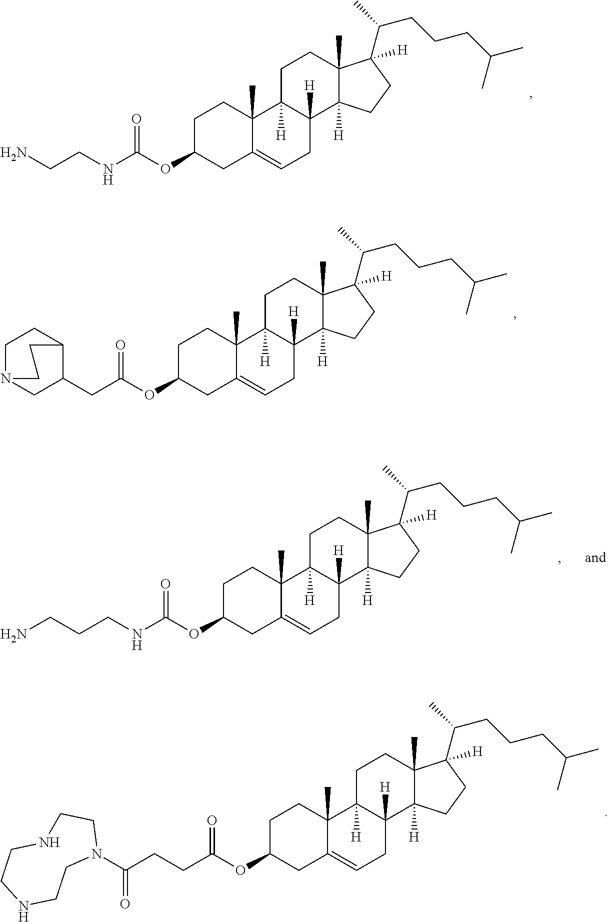

In some embodiments, R¹ is C₁₋₁₄ alkyl. In some embodiments, R¹ is C₃₋₁₂ alkyl. In some embodiments, R¹ is C₆₋₁₂ alkyl. In some embodiments, R¹ is C₈₋₁₀ alkyl. In some embodiments, R¹ is C₈ alkyl. In some embodiments, R¹ is C₁₀ alkyl.

In some embodiments, R¹ is C₁₋₁₄ hydroxyalkyl. In some embodiments, R¹ is C₃₋₁₂ hydroxyalkyl. In some embodiments, R¹ is C₆₋₁₂ hydroxyalkyl. In some embodiments, R¹ is C₈₋₁₀ hydroxyalkyl. In some embodiments, R¹ is C₈ hydroxyalkyl. In some embodiments, R¹ is C₁₀ hydroxyalkyl.

In some embodiments, R¹ is C₁₋₁₄ alkenyl. In some embodiments, R¹ is C₃₋₁₂ alkenyl. In some embodiments, R¹ is C₆₋₁₂ alkenyl. In some embodiments, R¹ is C₅₋₁₀ alkenyl. In some embodiments, R¹ is C₈ alkenyl. In some embodiments, R¹ is C₁₀ alkenyl.

In some embodiments, R¹ is

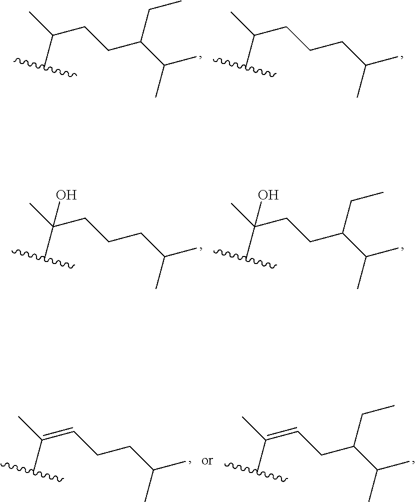

In some embodiments, R¹ is

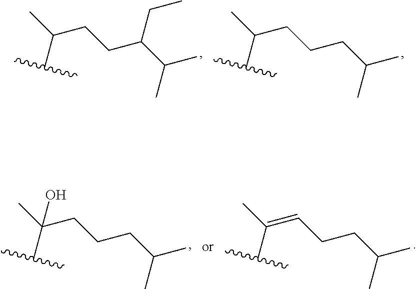

In some embodiments, R¹ is

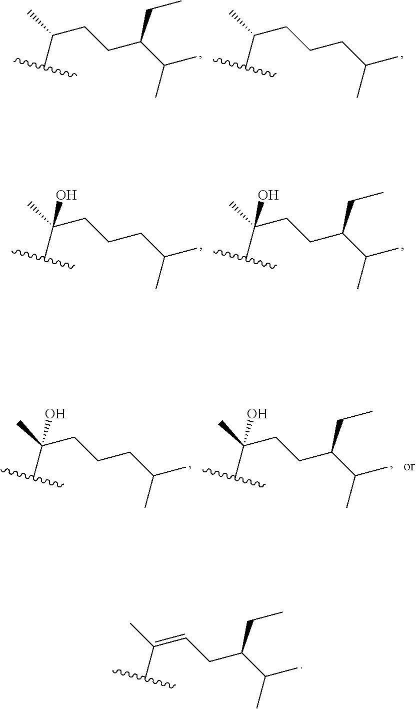

In some embodiments, R¹ is

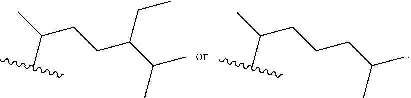

In some embodiments, R¹ is

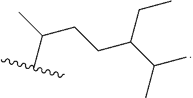 [SMILES: *C(C)CCC(CC)C(C)C ; "."]

In some embodiments, R¹ is

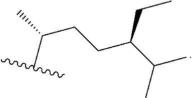 [SMILES: *[C@H](C)CC[C@@H](CC)C(C)C ; "."]

In some embodiments, A is —NR^{a}—. In some embodiments, A is —CR⁴R⁵—.

In some embodiments, R^{a} is H. In some embodiments, R^{a} is methyl.

In some embodiments, R⁴ and R⁵ are both H. In some embodiments, R⁴ and R⁵ are both C₁₋₄ alkyl. In some embodiments, R⁴ and R⁵ are both methyl. In some embodiments, one of R⁴ and R⁵ is H and the other of R⁴ and R⁵ is C₁₋₄ alkyl. In some embodiments, one of R⁴ and R⁵ is H and the other of R⁴ and R⁵ is methyl. In some embodiments, R⁴ and R⁵ together with the carbon atom to which they are attached form a C₃₋₅ cycloalkyl group. In some embodiments, R⁴ and R⁵ together with the carbon atom to which they are attached form a C₃ cycloalkyl group. In some embodiments, at least one R⁴ is C₁₋₄ alkyl. In some embodiments, at least one R⁴ is methyl.

In some embodiments, m is 0. In some embodiments, m is 1.

In some embodiments, D is —O—. In some embodiments, D is —S—S—.

In some embodiments, n is 0. In some embodiments, n is 1. In some embodiments, n is 2. In some embodiments, n is 3. In some embodiments, n is 4. In some embodiments, n is 5. In some embodiments, n is 0, 1, or 2.

In some embodiments, R⁶ and R⁷ are both H. In some embodiments, R⁶ and R⁷ are both C₁₋₄ alkyl. In some embodiments, R⁶ and R¹ are both methyl. In some embodiments, one of R⁶ and R⁷ is H and the other of R⁶ and R⁷ is C₁₋₄ alkyl. In some embodiments, one of R⁶ and R⁷ is H and the other of R⁴ and R⁵ is methyl. In some embodiments, R⁶ and R¹ together with the carbon atom to which they are attached form a C₃₋₅ cycloalkyl group. In some embodiments, R⁶ and R⁷ together with the carbon atom to which they are attached form a C₃ cycloalkyl group. In some embodiments, at least one R⁶ is C₁₋₄ alkyl. In some embodiments, at least one R⁶ is methyl.

In some embodiments, o is 0. In some embodiments, o is 1.

In some embodiments, E is —C(O)NH—. In some embodiments, E is —O—. In some embodiments, E is —C(O)—.

In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12. In some embodiments, p is 1, 2, 3, 4, 6, 8, or 10. In some embodiments, p is 2, 6, 8, or 10.

In some embodiments, R⁸ and R⁹ are both H. In some embodiments, R⁸ and R⁹ are both C₁₋₄ alkyl. In some embodiments, R⁸ and R⁹ are both methyl. In some embodiments, one of R⁸ and R⁹ is H and the other of R⁸ and R⁹ is C₁₋₄ alkyl. In some embodiments, one of R⁸ and R⁹ is H and the other of R⁸ and R⁹ is methyl. In some embodiments, R⁸ and R⁹ together with the carbon atom to which they are attached form a C₃₋₅ cycloalkyl group. In some embodiments, R⁸ and R⁹ together with the carbon atom to which they are attached form a C₃ cycloalkyl group. In some embodiments, at least one R¹ is C₁₋₄ alkyl. In some embodiments, at least one R¹ is methyl.

In some embodiments, n is 1, R⁶ is H, and R⁷ is H. In some embodiments, n is 2 and both R⁶ and R⁷ are H. In some embodiments, p is 1, R¹ is C₁₋₄ alkyl, and R⁹ is C₁₋₄ alkyl. In some embodiments, p is 1, R¹ is methyl, and R⁹ is methyl. In some embodiments, p is 1 and R⁸ and R⁹ together with the carbon atom to which they are attached form a C₃₋₅ cycloalkyl group. In some embodiments, p is 1 and R⁸ and R⁹ together with the carbon atom to which they are attached form a C₃ cycloalkyl group. In some embodiments, p is 2 and each R⁸ and R⁹ are H. In some embodiments, p is 3 and each R⁸ and R⁹ are H. In some embodiments, p is 4 and each R⁸ and R⁹ are H. In some embodiments, p is 6 and each of R⁸ and R⁹ are H. In some embodiments, p is 8 and each R⁸ and R⁹ are H. In some embodiments, p is 10 and each R⁸ and R⁹ are H.

In some embodiments, m is 0, n is 0, o is 0, and p is 2. In some embodiments, m is 0, n is 0, o is 0, and p is 3. In some embodiments, m is 0, n is 0, o is 0, and p is 4. In some embodiments, m is 0, n is 0, o is 0, and p is 8. In some embodiments, m is 0, n is 0, o is 0, and p is 10. In some embodiments, m is 0, n is 1, o is 0, and p is 1. In some embodiments, m is 0, n is 2, o is 1, and p is 2. In some embodiments, m is 1, n is 1, o is 1, and p is 2. In some embodiments, m is 1, n is 1, o is 1, and p is 6. In some embodiments, m is 1, n is 1, o is 1, and p is 8. In some embodiments, m is 1, n is 1, o is 1, and p is 10.

In some embodiments, m is 0, n is 0, o is 0, p is 2, and each R⁸ and R⁹ are H. In some embodiments, m is 0, n is 0, o is 0, p is 3, and each R⁸ and R⁹ are H. In some embodiments, m is 0, n is 0, o is 0, p is 4, and each R⁸ and R⁹ are H. In some embodiments, m is 0, n is 0, o is 0, p is 8, and each R⁸ and R⁹ are H. In some embodiments, m is 0, n is 0, o is 0, p is 10, and each R⁸ and R⁹ are H. In some embodiments, m is 0, n is 1, R⁶ is H, R⁷ is H, o is 0, p is 1, R¹ is C₁₋₄ alkyl, and R⁹ is C₁₋₄ alkyl. In some embodiments, m is 0, n is 1, R⁶ is H, R⁷ is H, o is 0, p is 1, R¹ is methyl, and R⁹ is methyl. In some embodiments, m is 0, n is 1, R⁶ is H, R⁷ is H, o is 0, p is 1, R⁸ and R⁹ together with the carbon atom to which they are attached form a C₃₋₅ cycloalkyl group. In some embodiments, m is 0, n is 1, R⁶ is H, R⁷ is H, o is 0, p is 1, R⁸ and R⁹ together with the carbon atom to which they are attached form a C₃ cycloalkyl group. In some embodiments, m is 0, n is 2, each of R⁶ and R⁷ are H, o is 1, E is —O—, p is 2, and each of R⁸ and R⁹ are H. In some embodiments, m is 1, n is 1, R⁶ is H, R⁷ is H, o is 1, E is —C(O)NH—, p is 2, and each of R⁸ and R⁹ are H. In some embodiments, m is 1, n is 1, R⁶ is H, R⁷ is H, o is 1, E is —C(O)NH—, p is 6, and each of R⁸ and R⁹ are H. In some embodiments, m is 1, n is 1, R⁶ is H, R⁷ is H, o is 1, E is —C(O)NH—, p is 8, and each of R¹ and R⁹ are H. In some embodiments, m is 1, n is 1, R⁶ is H, R⁷ is H, o is 1, E is —C(O)NH—, p is 10, and each of R⁸ and R⁹ are H.

In some embodiments, m is 0, n is 0, o is 0, p is 1, and R⁸ with R² and R³ together with the atoms to which they are attached and any intervening atoms, form a 7-18 membered bridged heterocycloalkyl group and R⁹ is H. In some embodiments, m is 0, n is 0, o is 0, p is 1, and R¹ with R² and R³ together with the atoms to which they are attached and any intervening atoms, form a 7-12 membered bridged heterocycloalkyl group and R⁹ is H. In some embodiments, m is 0, n is 0, o is 0, p is 1, and R¹ with R² and R³ together with the atoms to which they are attached and any intervening atoms, form a 8 membered bridged heterocycloalkyl group and R⁹ is H. In some embodiments, m is 0, n is 0, o is 0, p is 1, and R⁹ is H and R⁸ with R² and R³ together with the atoms to which they are attached and any intervening atoms, form a 7-18 membered bridged heterocycloalkyl group having the formula:

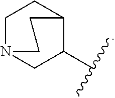 [SMILES: *C1CN2CCC1CC2 ; "."]

In some embodiments, R² and R³ are both H. In some embodiments, R² and R³ are both methyl. In some embodiments, R² and R³ are both methyl substituted by —OH. In some embodiments, R² and R³ are both ethyl. In some embodiments, R² and R³ are both ethyl substituted by —OH.

In some embodiments, one of R² and R³ is H and the other of R² and R³ is methyl. In some embodiments, one of R² and R³ is H and the other of R² and R³ is methyl substituted with —OH. In some embodiments, one of R² and R³ is H and the other of R² and R³ is ethyl. In some embodiments, one of R² and R³ is H and the other of R² and R³ is ethyl substituted with —OH.

In some embodiments, one of R² and R³ is methyl and the other is ethyl. In some embodiments, one of R² and R³ is methyl substituted with OH and the other of R² and R³ is ethyl. In some embodiments, one of R² and R³ is methyl and the other of R² and R³ is ethyl substituted with OH. In some embodiments, one of R² and R³ is methyl substituted with —OH and the other of R² and R³ is ethyl substituted with —OH.

In some embodiments, both R² and R³ are

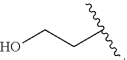 [SMILES: *CCO ; "."]

In some embodiments, one of R² and R³ is methyl and the other of R² and R³ is

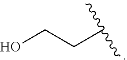 [SMILES: *CCO ; "."]

In some embodiments, R², R³, and R⁸, together with the atoms to which they are attached and any intervening atoms, form a 7-18 membered bridged heterocycloalkyl group. In some embodiments, R², R³, and R⁸, together with the atoms to which they are attached and any intervening atoms, form a 7-12 membered bridged heterocycloalkyl group. In some embodiments, R², R³, and R⁸, together with the atoms to which they are attached and any intervening atoms, form an 8 membered bridged heterocycloalkyl group. In some embodiments, R², R³, and R⁶, together with the atoms to which they are attached and any intervening atoms, form a 7-18 membered bridged heterocycloalkyl group having the formula:

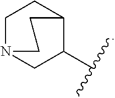 [SMILES: *C1CN2CCC1CC2 ; "."]

In some embodiments, R² and R³ together with the N atom to which they are attached form a 7-18 membered heterocycloalkyl group comprising 1, 2, or 3 ring-forming —NR¹⁰— groups, wherein the 7-18 membered heterocycloalkyl group is optionally substituted with 1, 2, or 3 substituents independently selected from C₁₋₄ alkyl, —NR⁸R⁹, —OH, and halo.

In some embodiments, R² and R³ together with the N atom to which they are attached form a 7-12 membered heterocycloalkyl group comprising 1, 2, or 3 ring-forming —NR¹⁰— groups, wherein the 7-12 membered heterocycloalkyl group is optionally substituted with 1, 2, or 3 substituents independently selected from C₁₋₄ alkyl, —NR⁸R⁹, —OH, and halo.

In some embodiments, R² and R³ together with the N atom to which they are attached form a 8-10 membered heterocycloalkyl group comprising 1, 2, or 3 ring-forming —NR¹⁰— groups, wherein the 8-10 membered heterocycloalkyl group is optionally substituted with 1, 2, or 3 substituents independently selected from C₁₋₄ alkyl, —NR⁸R⁹, —OH, and halo.

In some embodiments, R² and R³ together with the N atom to which they are attached form a 8-10 membered heterocycloalkyl group comprising 1, 2, or 3 ring-forming —NCH₃— or —NH— groups, wherein the 8-10 membered heterocycloalkyl group is optionally substituted with 1, 2, or 3 substituents independently selected from C₁₋₄ alkyl, —NR⁸R⁹, —OH, and halo.

In some embodiments, R² and R³ together with the N atom to which they are attached form an 8-10 membered heterocycloalkyl group comprising 1, 2, or 3 ring-forming —NCH₃— or —NH— groups.

In some embodiments, R² and R³ together with the N atom to which they are attached form a heterocycloalkyl group of formula:

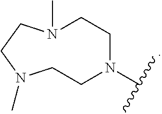 [SMILES: *N1CCN(C)CCN(C)CC1 ; "."]

In some embodiments:
A is —NR^{a}— or —CR⁴R⁵—;
D is —S—S—;
E is —C(O)—, —C(O)NH—, or —O—;
R¹ is C₁₋₁₄ alkyl;
R² and R³ are each independently selected from H, methyl, and ethyl substituted by OH;
or R² and R³ together with the N atom to which they are attached form a 7-18 membered heterocycloalkyl group comprising two ring-forming —NR¹⁰— groups;
or R², R³, and R⁸, together with the atoms to which they are attached and any intervening atoms, form a 7-18 membered bridged heterocycloalkyl group;
R^{a} is H;
R⁴, R⁵, R⁶, and R⁷ are each H;
R⁸ and R⁹ are each independently selected from H and C₁₋₄ alkyl;
or R⁸ and R⁹ together with the carbon atom to which they are attached form a C₃₋₅ cycloalkyl group;
R¹⁰ is C₁₋₄ alkyl;
m is 0 or 1;
n is 0, 1, or 2;
is 0 or 1; and
p is 0, 1, 2, 3, 4, 6, 8, or 10,
wherein at least one of m, n, o, and p is other than 0;
wherein p is 1 when R², R³, and R⁸, together with the atoms to which they are attached and any intervening atoms, form a 7-18 membered bridged heterocycloalkyl group; and
wherein when m is 1, then A is —CR⁴R⁵— and n is 1.

In some embodiments:
A is —NR^{a}— or —CR⁴R⁵;
D is —S—S—;
E is —C(O)—, —C(O)NH—, or —O—;
R¹ is C₁₋₁₄ alkyl;
R² and R³ are each independently selected from H, methyl, and ethyl substituted by —OH;
or R² and R³ together with the N atom to which they are attached form a 7-18 membered heterocycloalkyl group comprising two ring-forming —NR¹⁰— groups;
or R², R³, and R⁸, together with the atoms to which they are attached and any intervening atoms, form a 7-18 membered bridged heterocycloalkyl group;
R^{a} is H or methyl;
R⁴, R⁵, R⁶, and R⁷ are each H;
R⁸ and R⁹ are each independently selected from H and C₁₋₄ alkyl;
or R⁸ and R⁹ together with the carbon atom to which they are attached form a C₃₋₅ cycloalkyl group;
R¹⁰ is C₁₋₄ alkyl;
m is 0 or 1;
n is 0, 1, or 2;
o is 0 or 1; and
p is 0, 1, 2, 3, 4, 6, 8, or 10,
wherein at least one of m, n, o, and p is other than 0;
wherein p is 1 when R², R³, and R⁸, together with the atoms to which they are attached and any intervening atoms, form a 7-18 membered bridged heterocycloalkyl group; and
wherein when m is 1, then A is —CR⁴R⁵— and n is 1.

In some embodiments, the compound of Formula A8 is a compound of Formula A9:

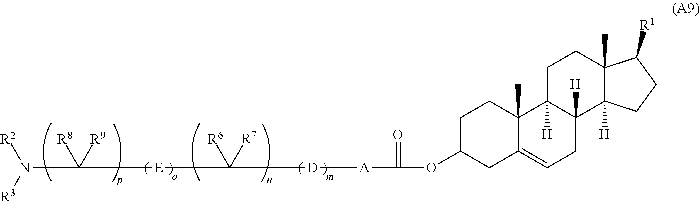

or a salt thereof.

In some embodiments, the sterol amine is selected from:

TABLE 1

| Sterol | |
|---|---|
| amine no. | Structure |
| SA1 | 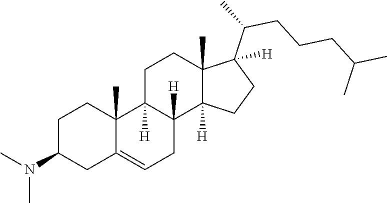 |
| SA2 | 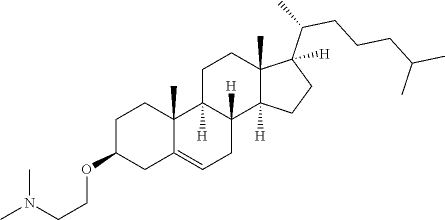 |
| SA3 | 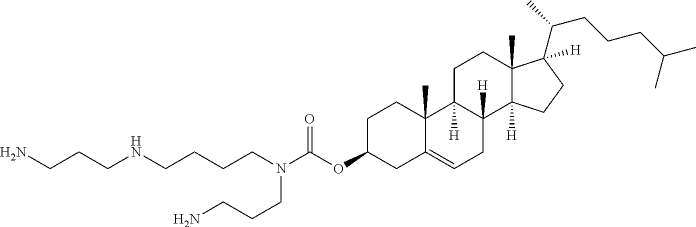 |
| SA4 | 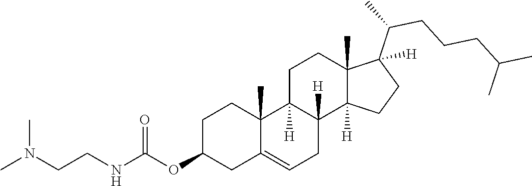 |
| SA5 | 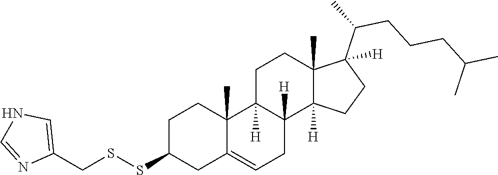 |
| SA6 | 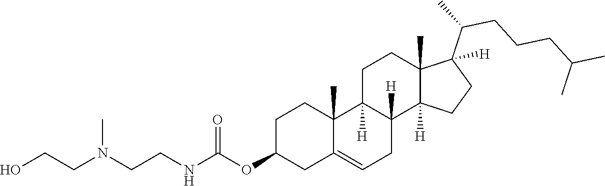 |
| SA7 | 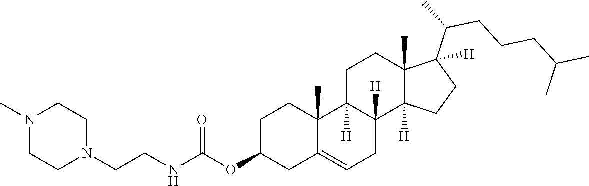 |
| SA8 | 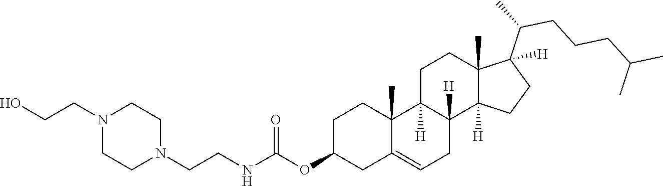 |
| SA9 | 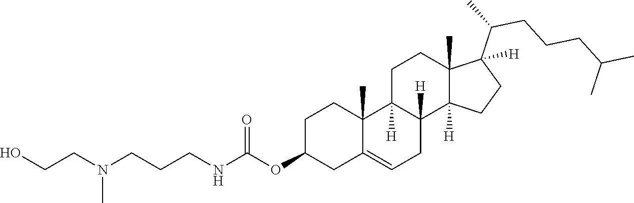 |
| SA10 | 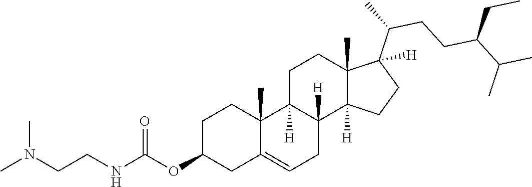 |
| SA11 | 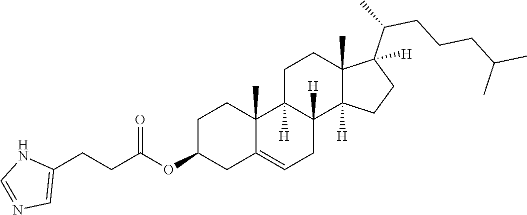 |
| SA12 | 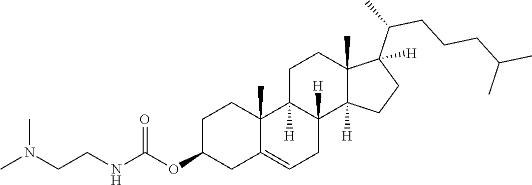 |
| SA13 | 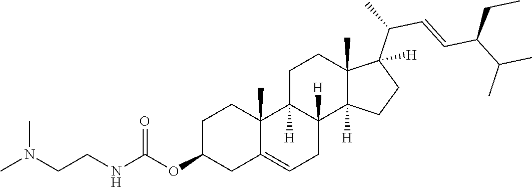 |
| SA14 | 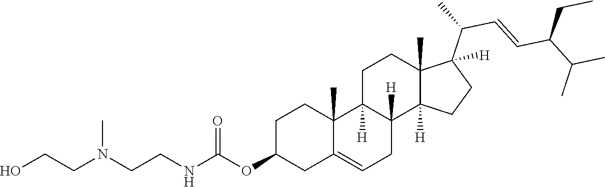 |
| SA15 | 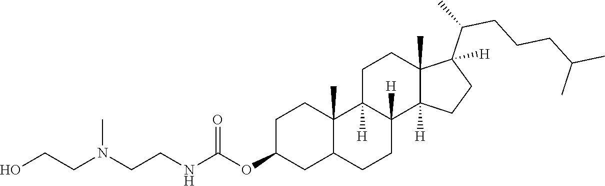 |
| SA16 | 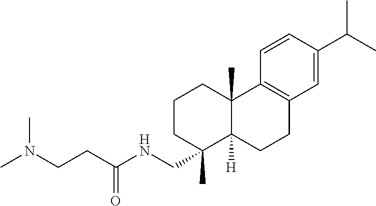 [SMILES: CC(C)c1ccc2c(c1)CC[C@H]1[C@](C)(CNC(=O)CCN(C)C)CCC[C@]21C] |
| SA17 | 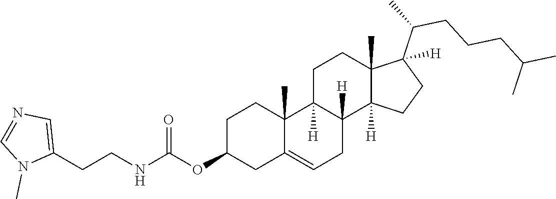 |
| SA18 | 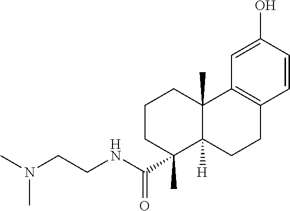 [SMILES: CN(C)CCNC(=O)[C@]1(C)CCC[C@]2(C)c3cc(O)ccc3CC[C@@H]12] |
| SA19 | 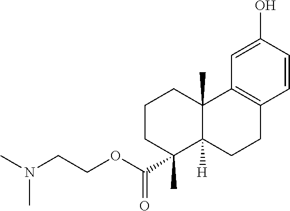 [SMILES: CN(C)CCOC(=O)[C@]1(C)CCC[C@]2(C)c3cc(O)ccc3CC[C@@H]12] |
| SA20 | 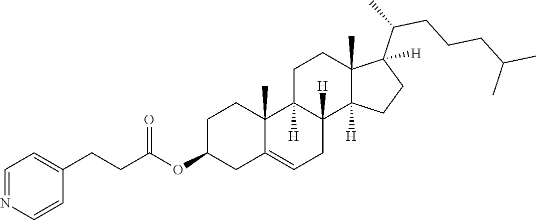 |
| SA21 | 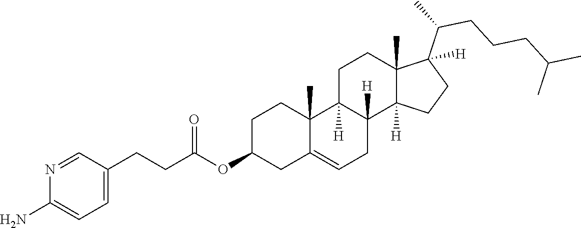 |
| SA22 | 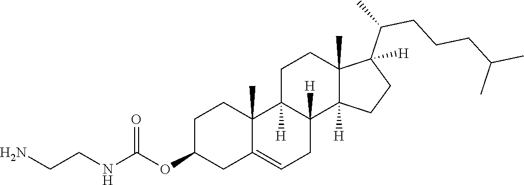 |
| SA23 | 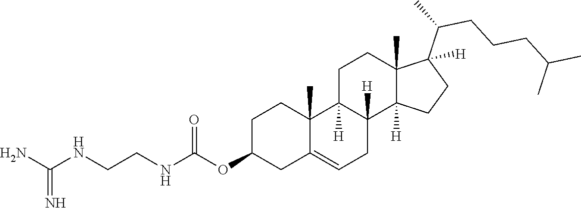 |
| SA24 | 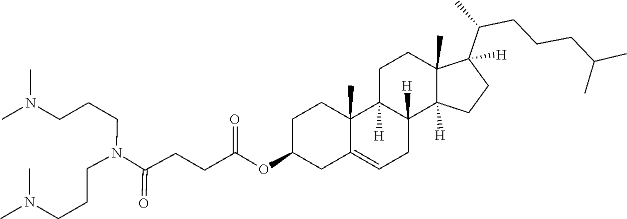 |
| SA25 | 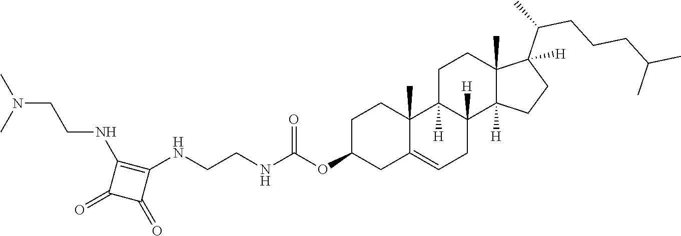 |
| SA26 | 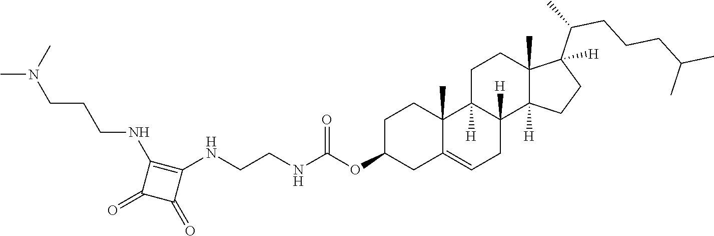 |
| SA27 | 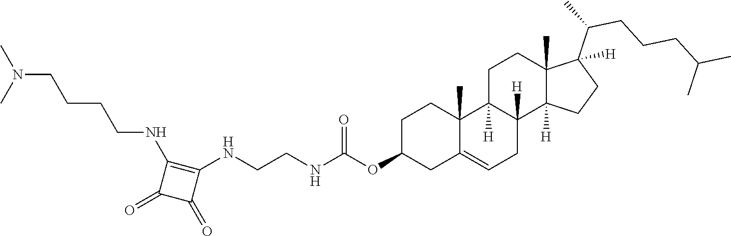 |
| SA28 | 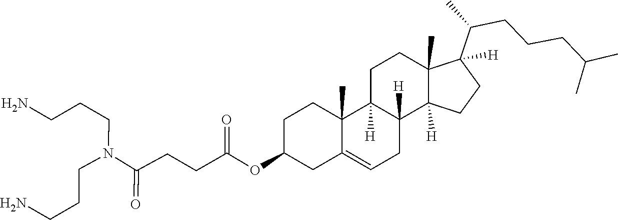 |
| SA29 | 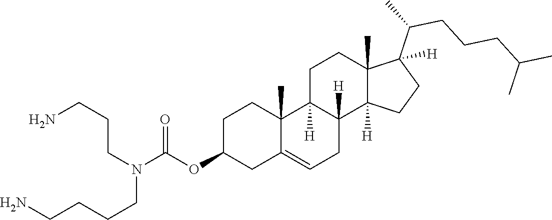 |
| SA30 | 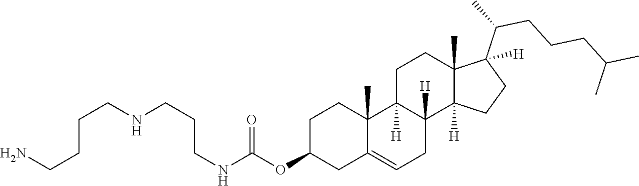 |
| SA31 | 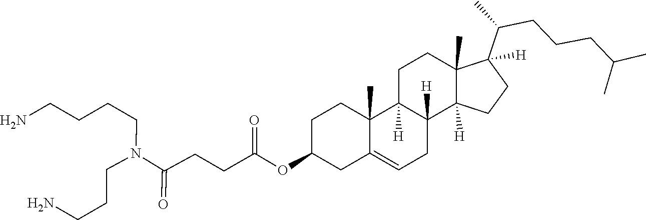 |
| SA32 | 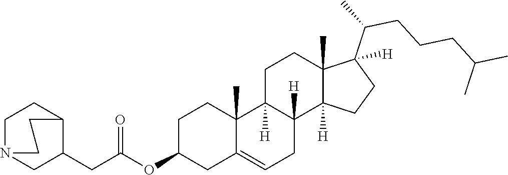 |
| SA33 | 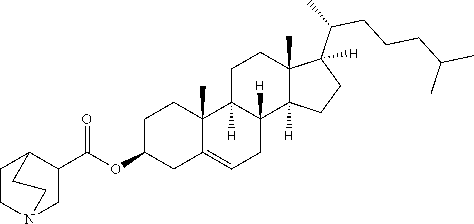 |
| SA34 | 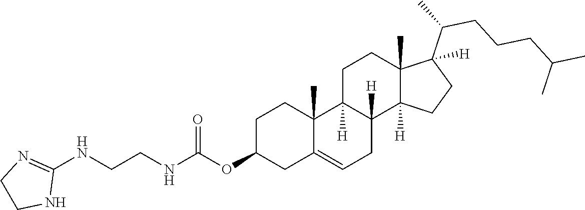 |
| SA35 | 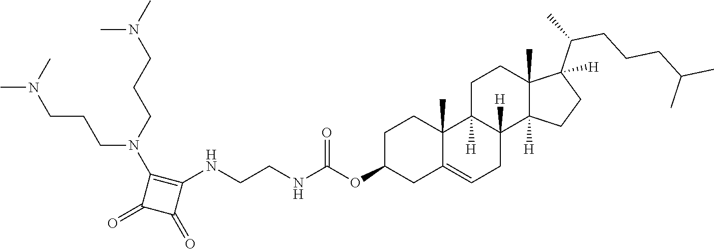 |
| SA36 | 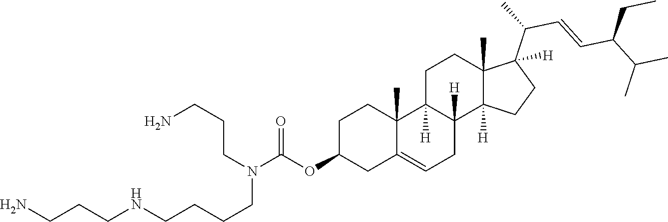 |
| SA37 | 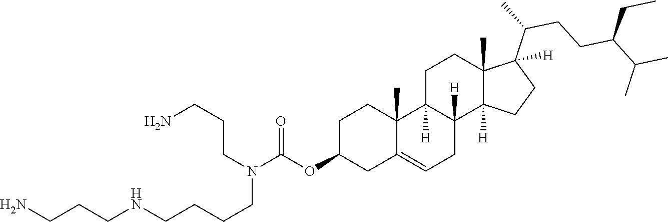 |
| SA38 | 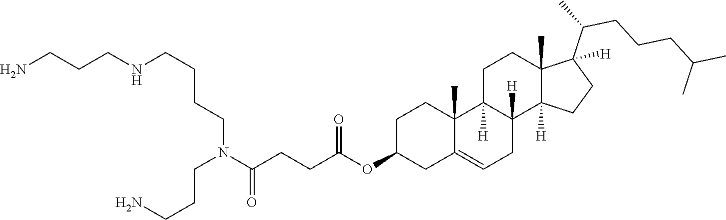 |
| SA39 | 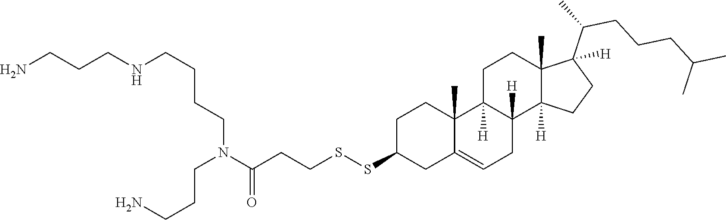 |
| SA40 | 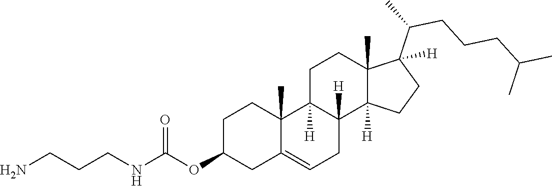 |
| SA41 | 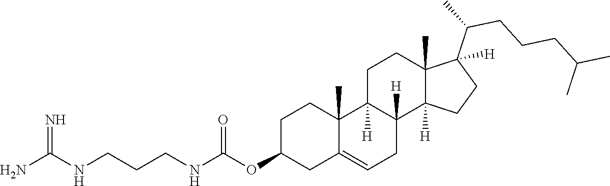 |
| SA42 | 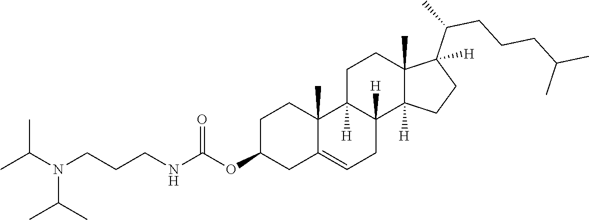 |
| SA43 | 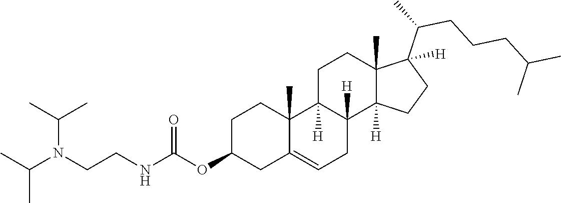 |
| SA44 | 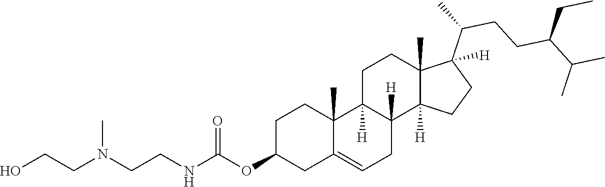 |
| SA45 | 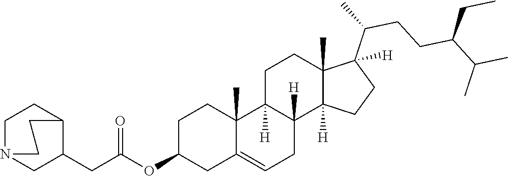 |
| SA47 | 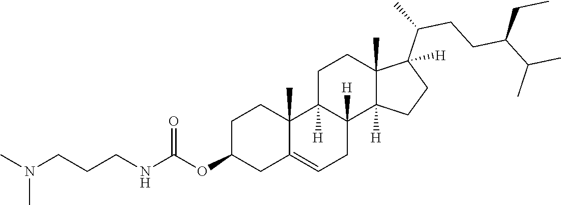 |
| SA48 | 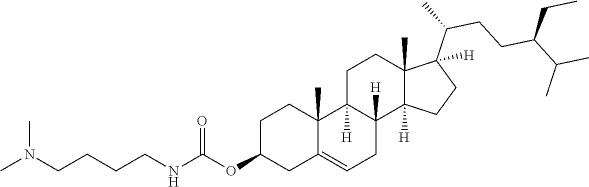 |
| SA49 | 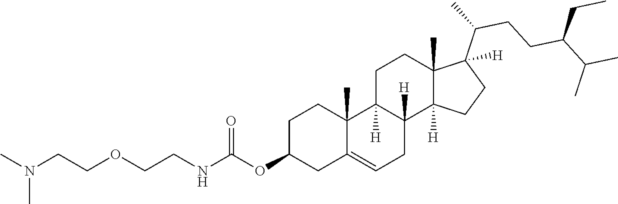 |

or a salt thereof.

In some embodiments, the sterol amine is selected from:

TABLE 2

| Sterol | |
|---|---|
| amine | |
| no. | Structure |
| SA50 | 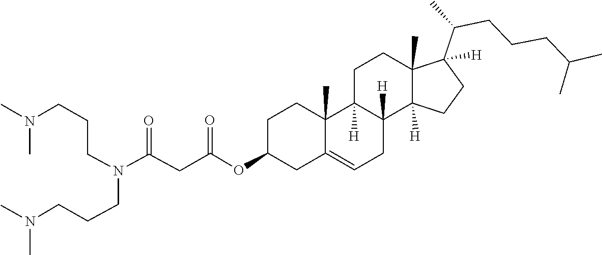 |
| SA51 | 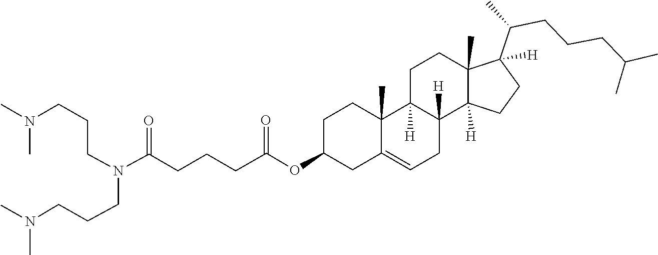 |
| SA52 | 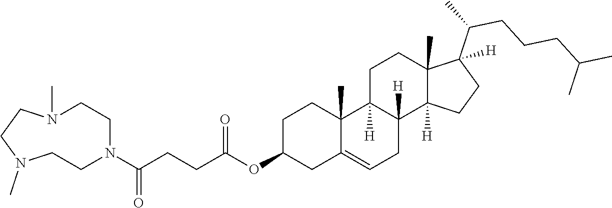 |
| SA53 | 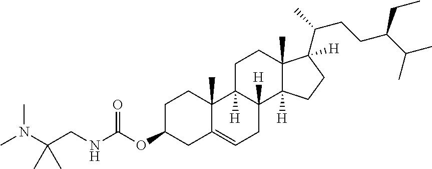 |
| SA54 | 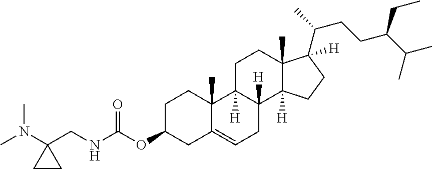 |
| SA55 | 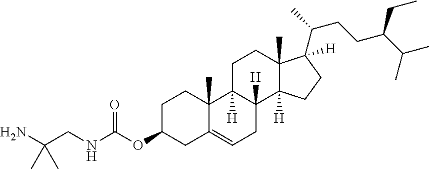 |
| SA56 | 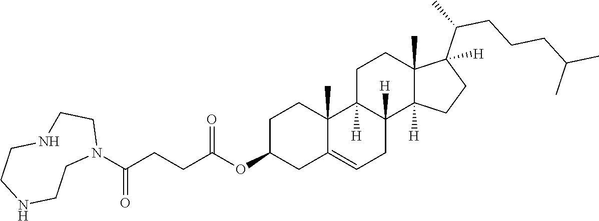 |
| SA57 | 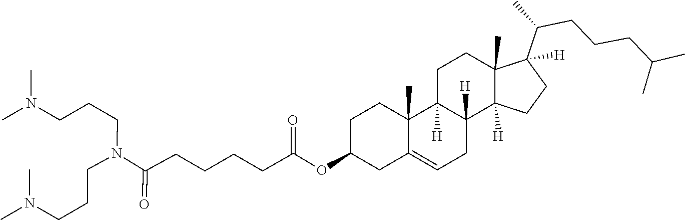 |
| SA58 | 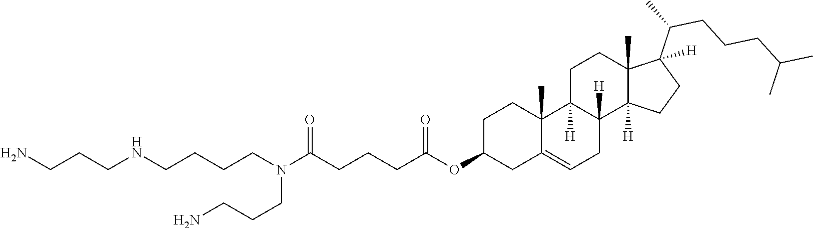 |
| SA59 | 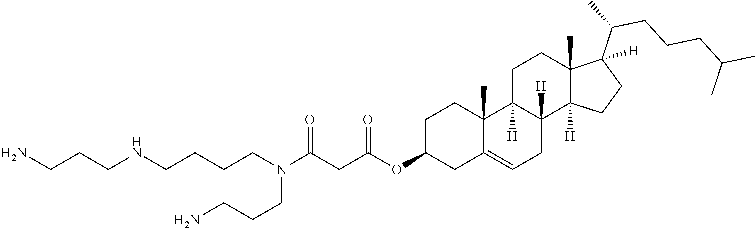 |
| SA60 | 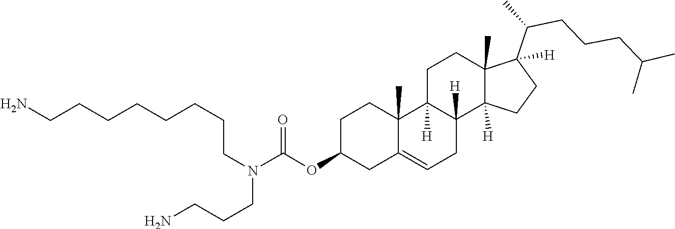 |
| SA61 | 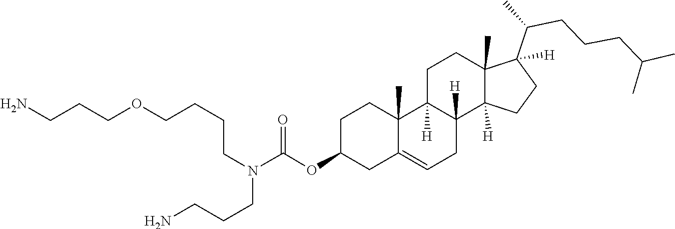 |
| SA62 | 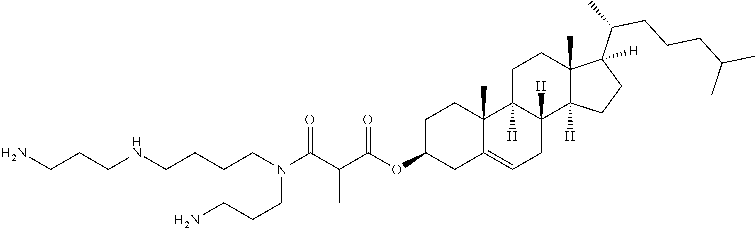 |
| SA63 | 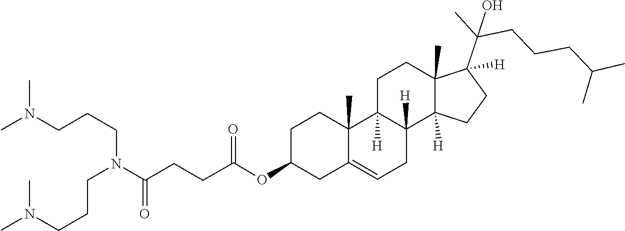 |
| SA64 | 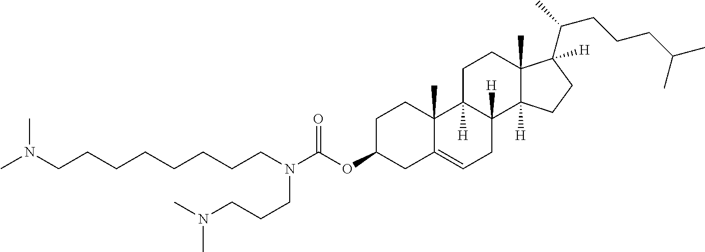 |
| SA65 | 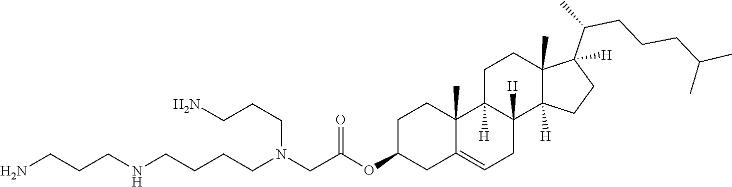 |
| SA66 | 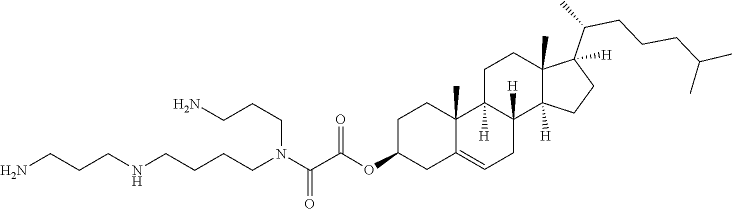 |
| SA67 | 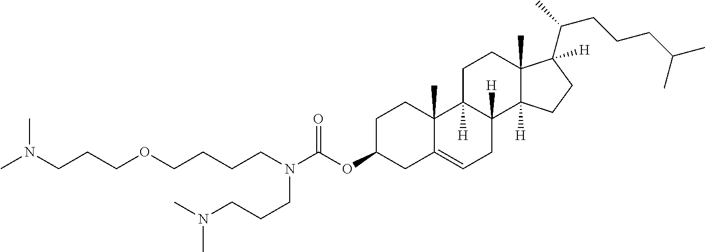 |
| SA68 | 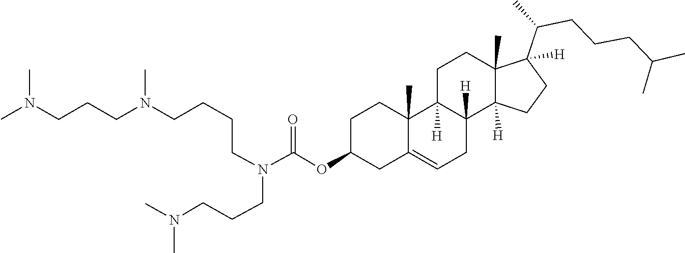 |
| SA69 | 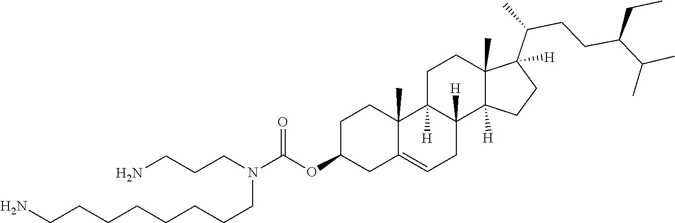 |
| SA70 | 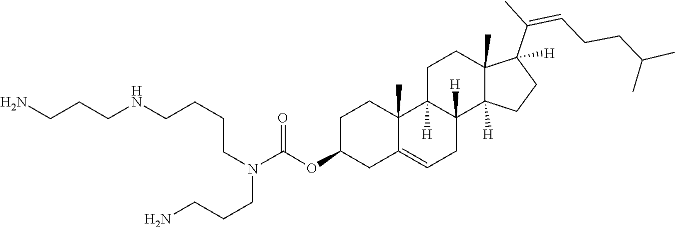 |
| SA71 | 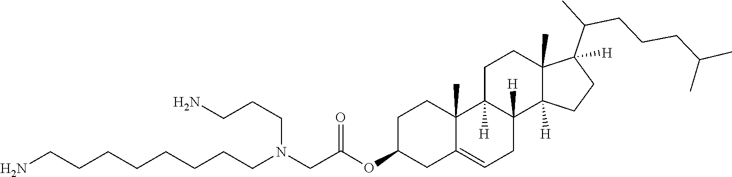 |
| SA72 | 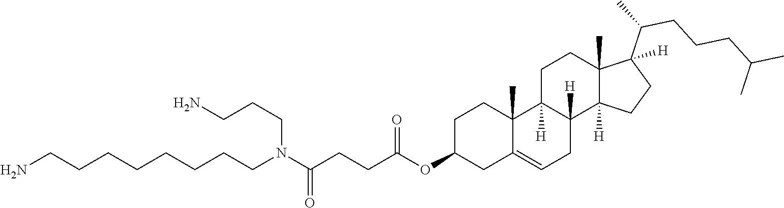 |
| SA73 | 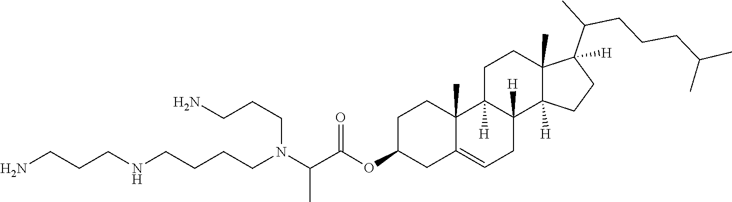 |
| SA74 | 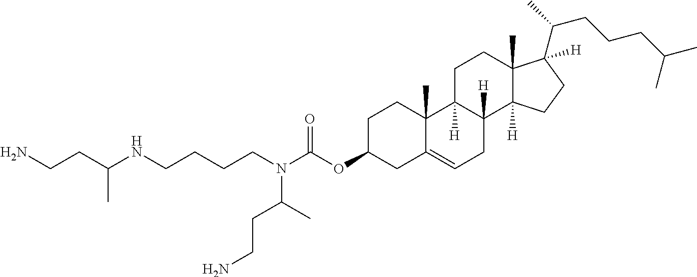 |
| SA75 | 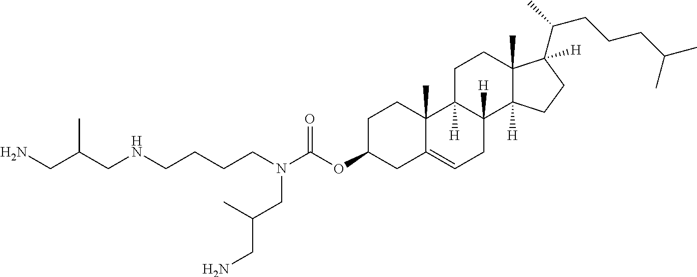 |
| SA76 | 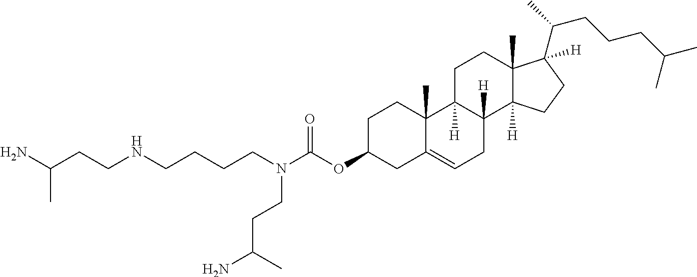 |
| SA77 | 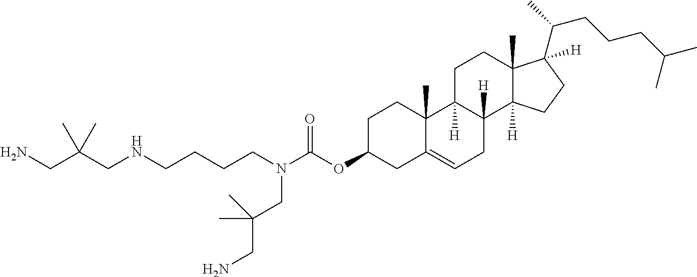 |
| SA78 | 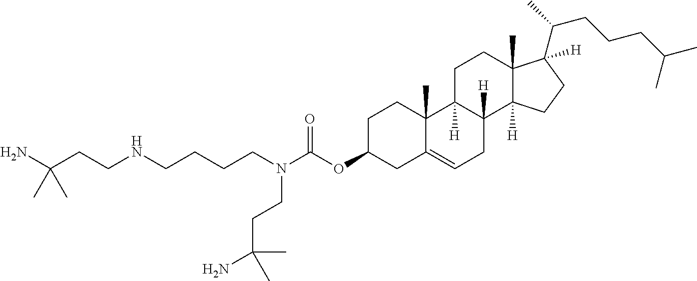 |
| SA79 | 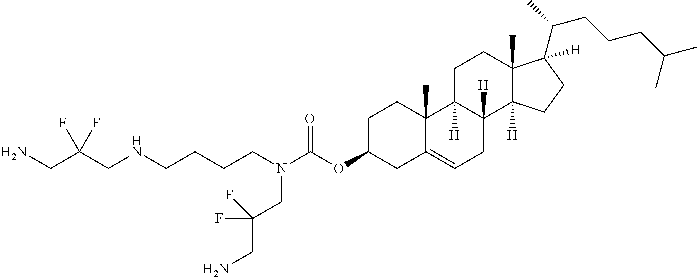 |
| SA81 | 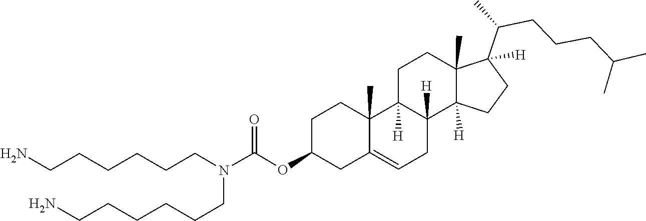 |
| SA82 | 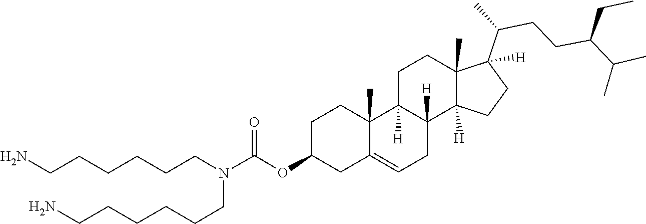 |
| SA83 | 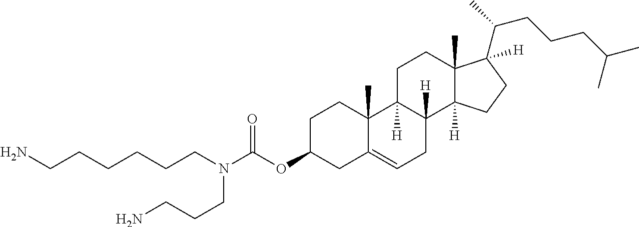 |
| SA84 | 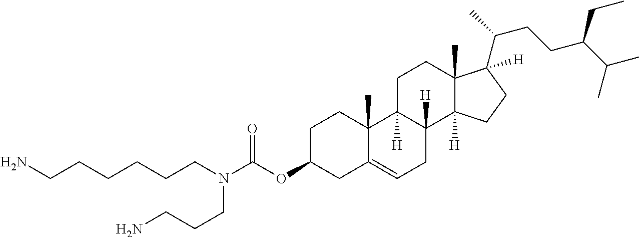 |
| SA85 | 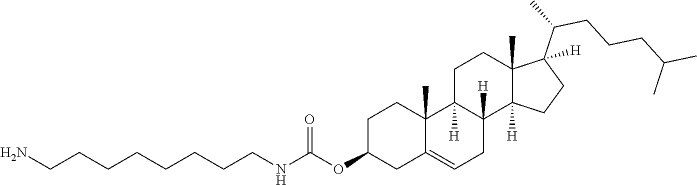 |
| SA86 | 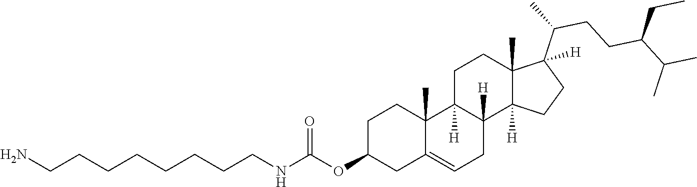 |
| SA87 | 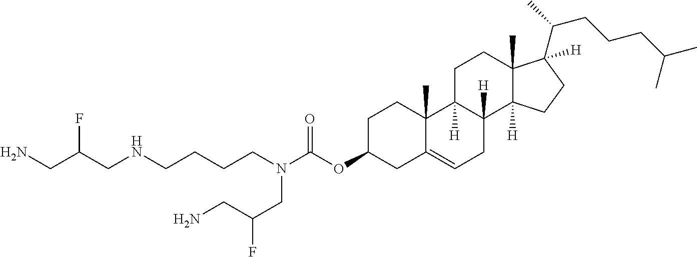 |
| SA88 | 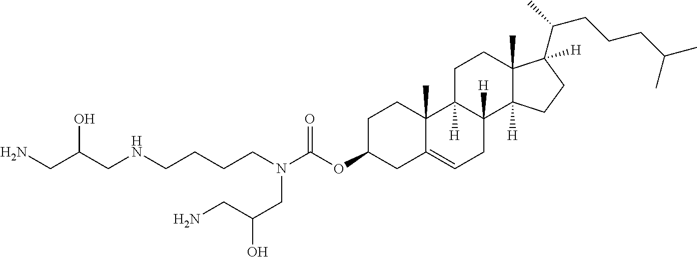 |
| SA89 | 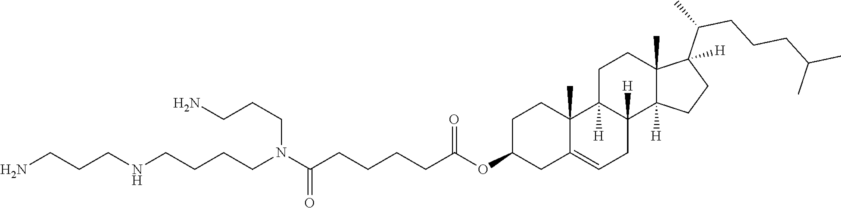 |
| SA90 | 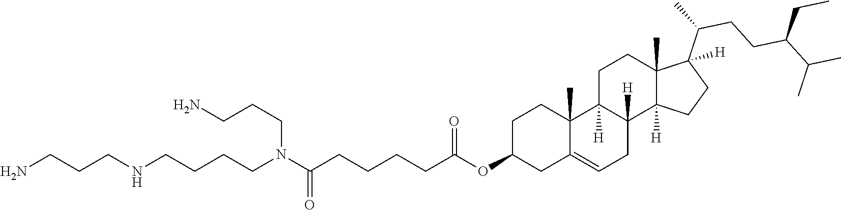 |
| SA91 | 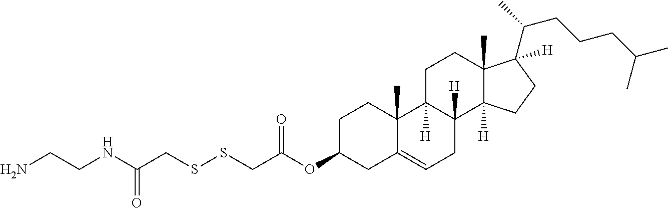 |
| SA92 | 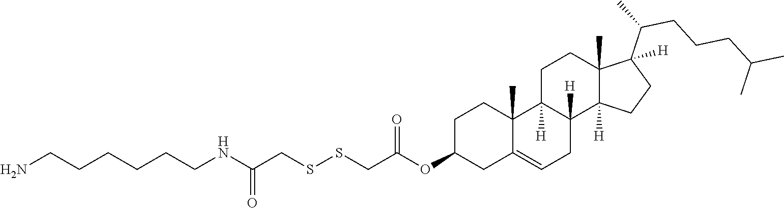 |
| SA93 | 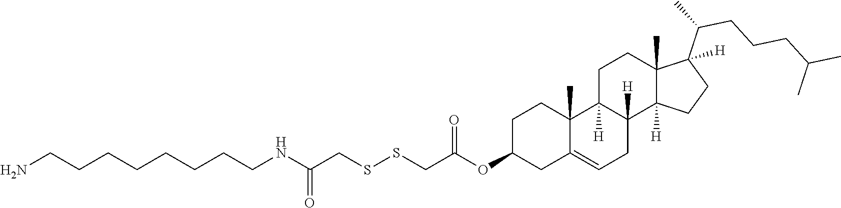 |
| SA94 | 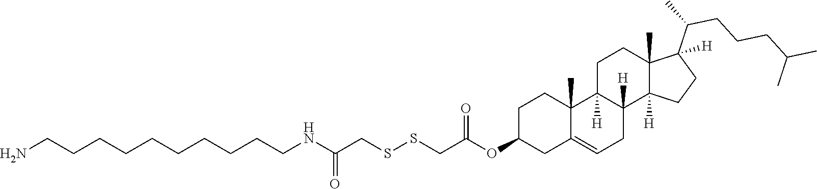 |
| SA95 | 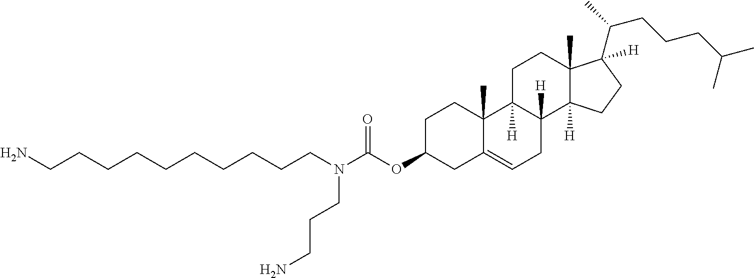 |
| SA96 | 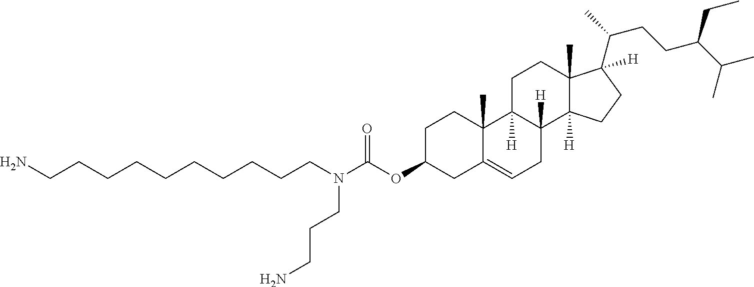 |
| SA97 | 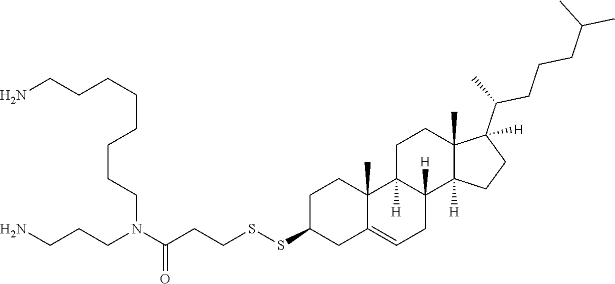 |
| SA98 | 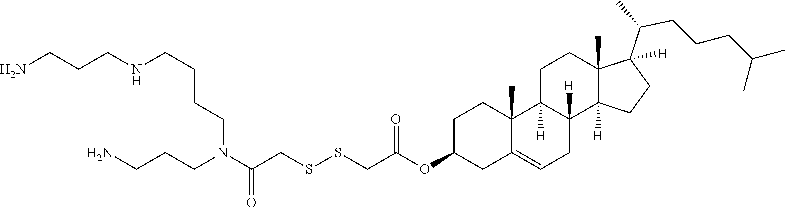 |
| SA110 | 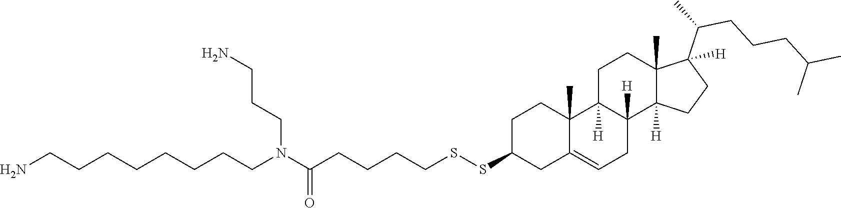 |
| SA111 | 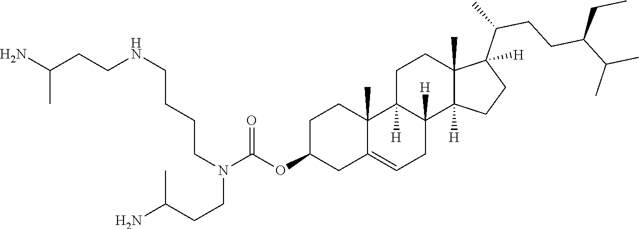 |
| SA113 | 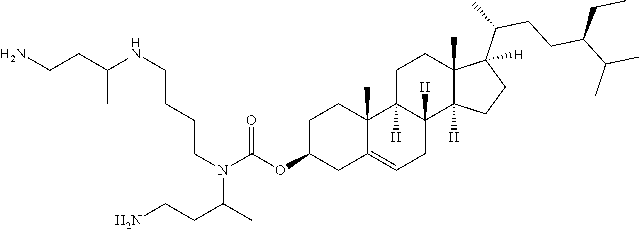 |
| SA114 | 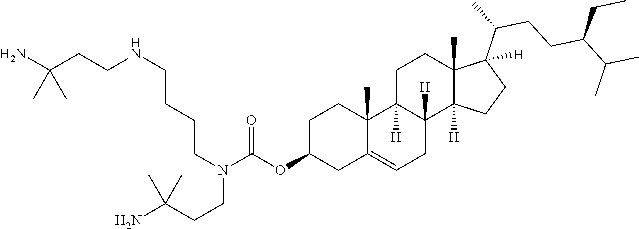 |
| SA116 | 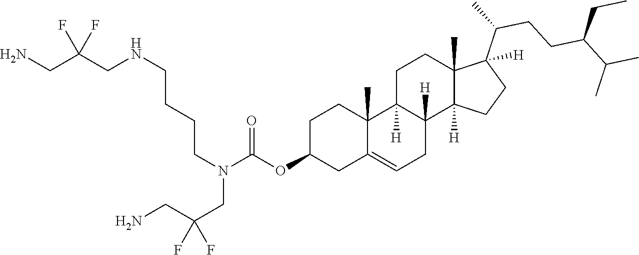 |
| SA117 | 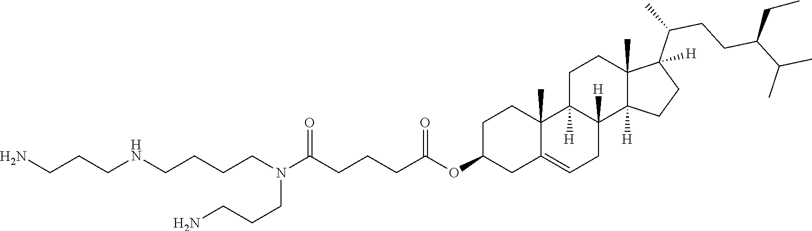 |
| SA118 | 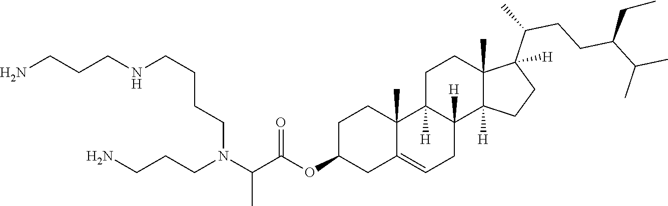 |
| SA119 | 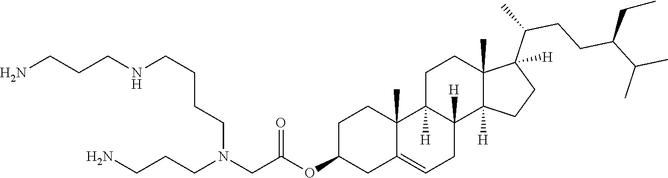 |
| SA120 | 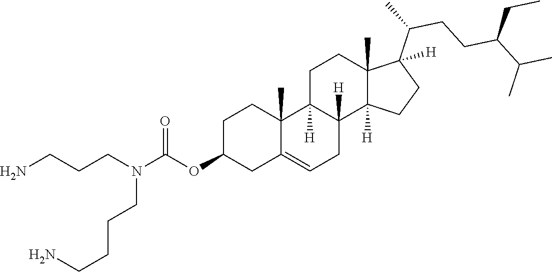 |
| SA121 | 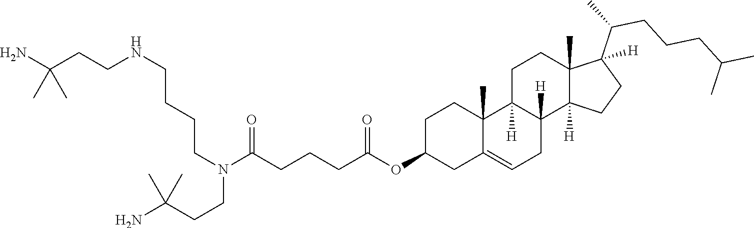 |
| SA122 | 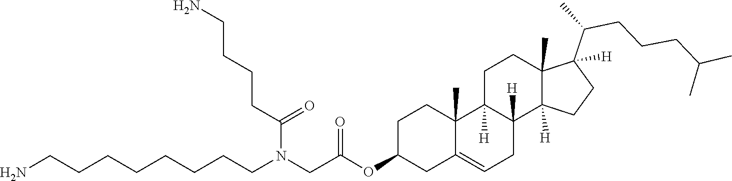 |
| SA123 | 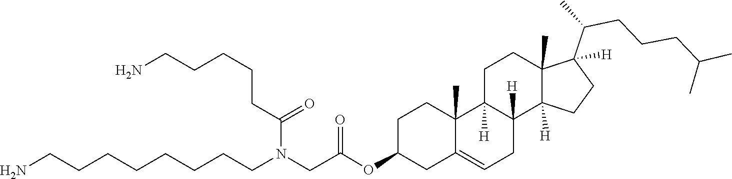 |
| SA124 | 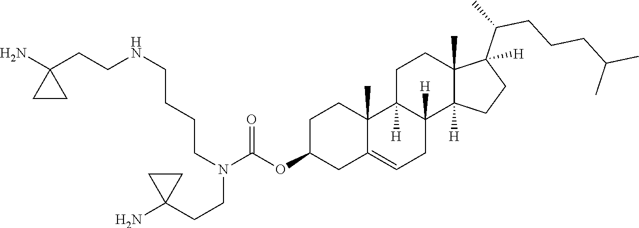 |
| SA125 | 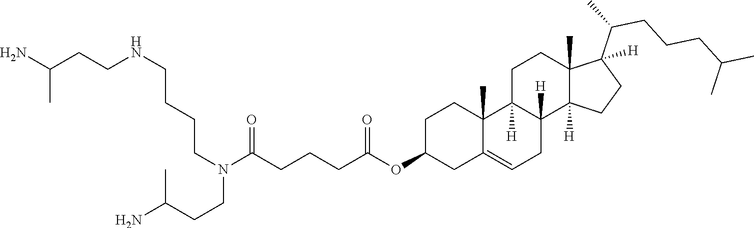 |
| SA126 | 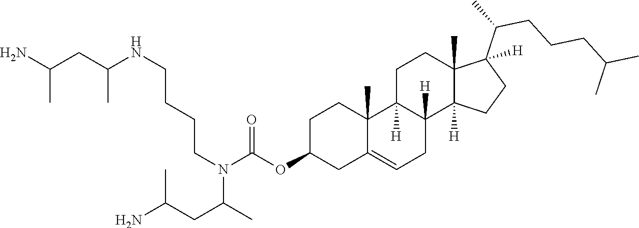 |
| SA127 | 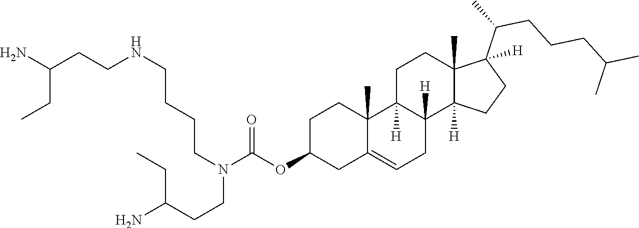 |
| SA128 | 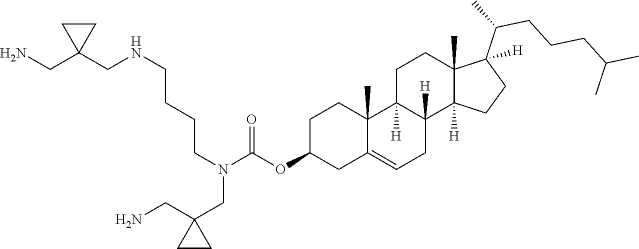 |
| SA129 | 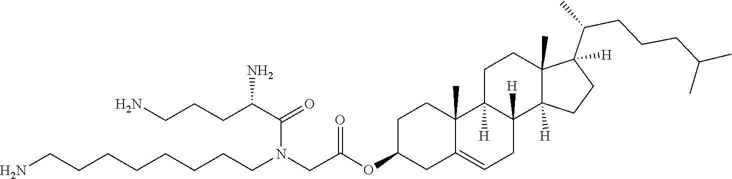 |
| SA130 | 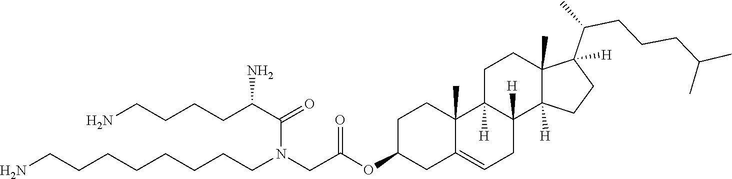 |
| SA131 | 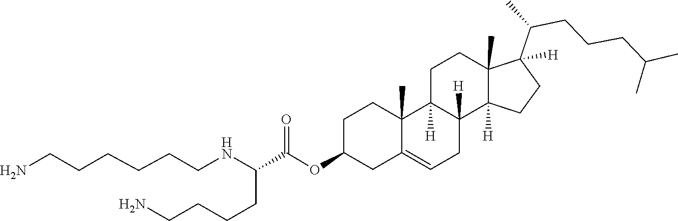 |
| SA132 | 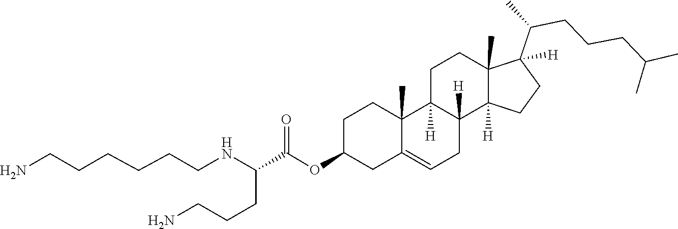 |
| SA133 | 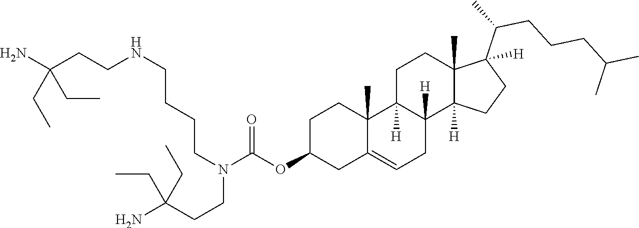 |
| SA134 | 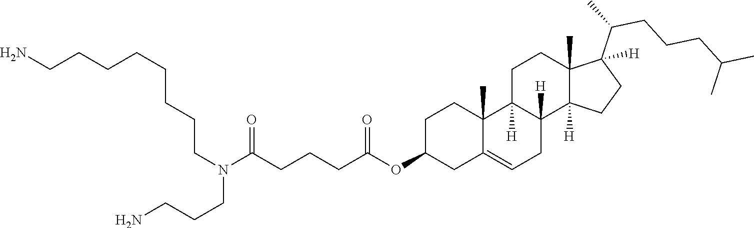 |
| SA135 | 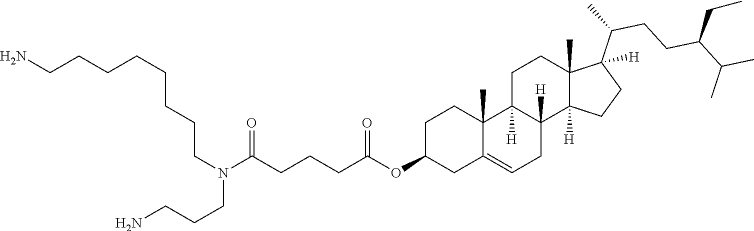 |
| SA136 | 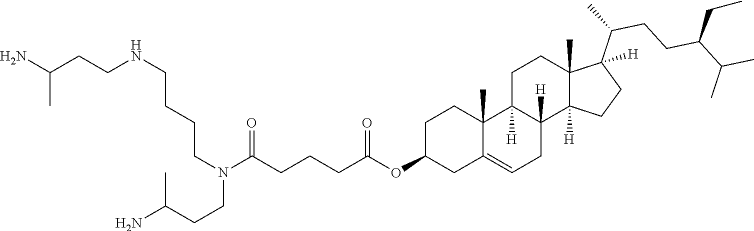 |
| SA137 | 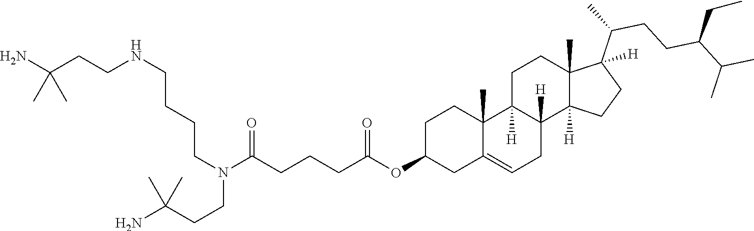 |
| SA138 | 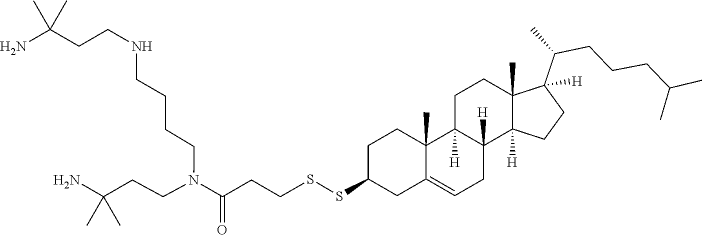 |
| SA139 | 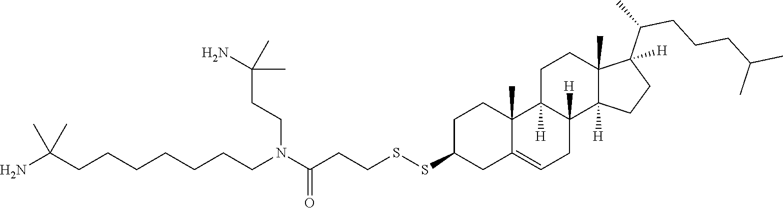 |
| SA141 | 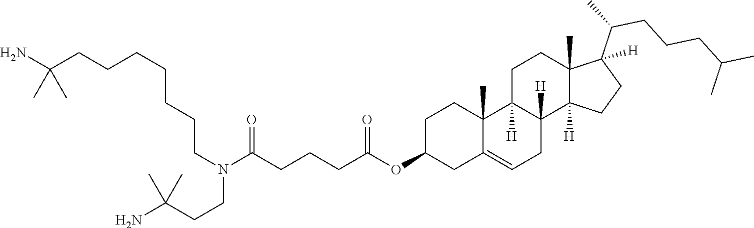 |
| SA142 | 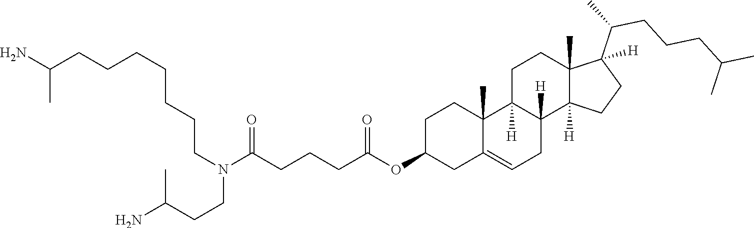 |
| SA144 | 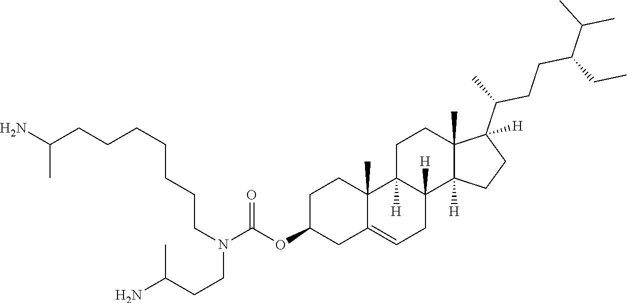 |
| SA145 | 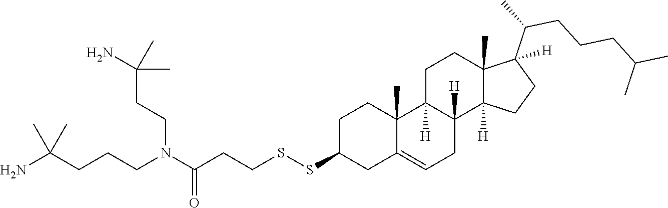 |
| SA149 | 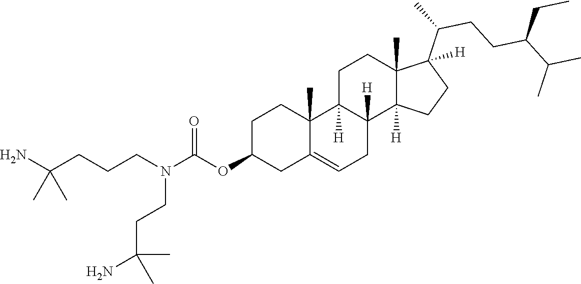 |
| SA151 | 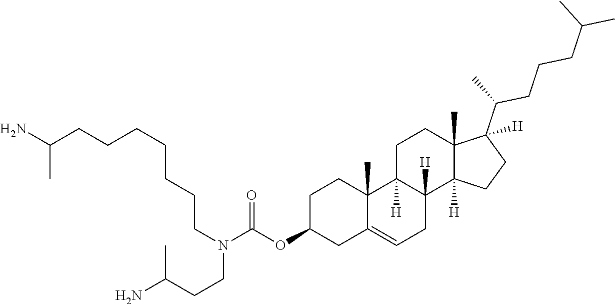 |
| SA152 | 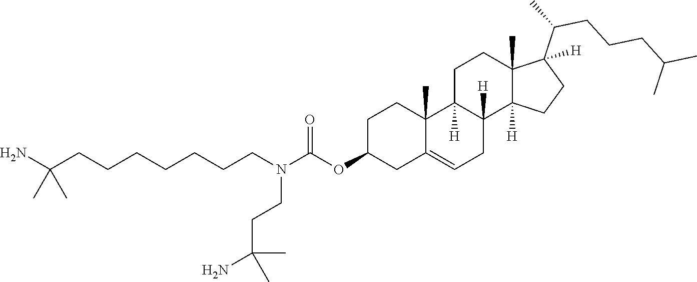 |
| SA153 | 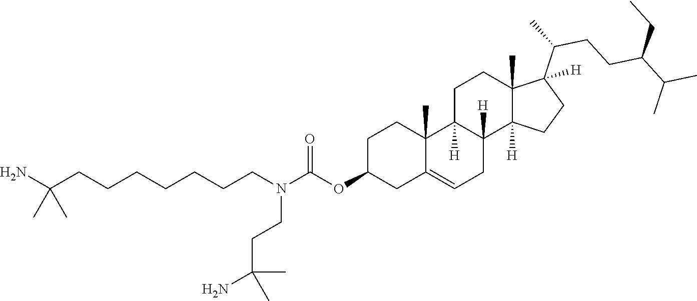 |
| SA154 | 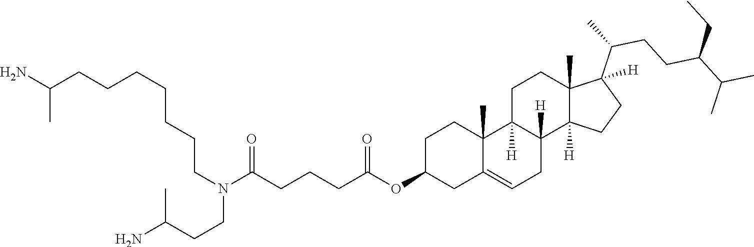 |
| SA155 | 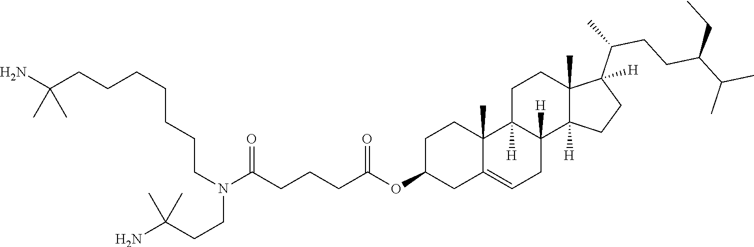 |
| SA156 | 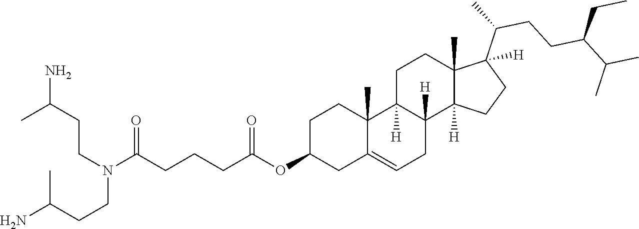 |
| SA157 | 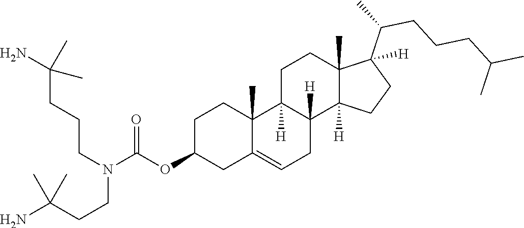 |
| SA158 | 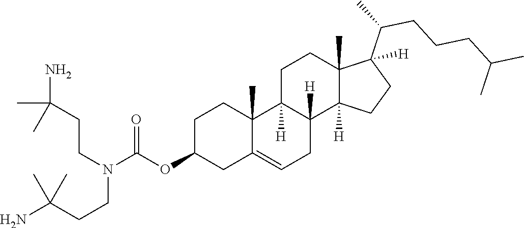 |
| SA159 | 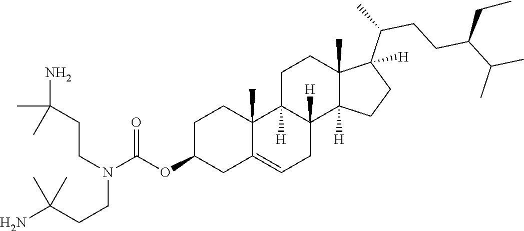 |
| SA160 | 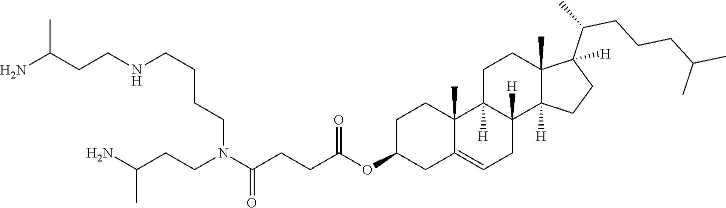 |
| SA161 | 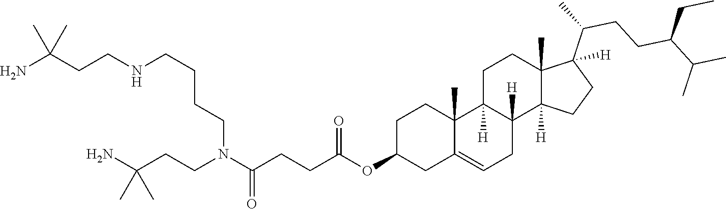 |
| SA162 | 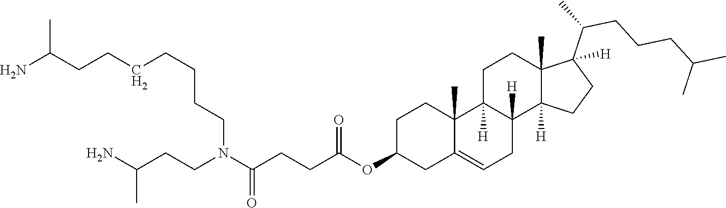 |
| SA163 | 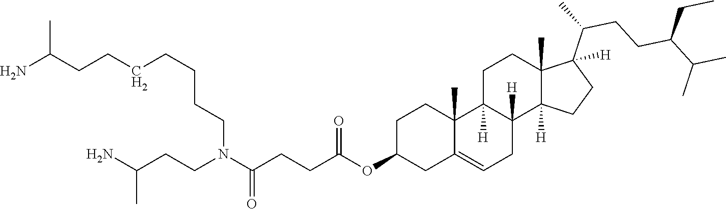 |
| SA164 | 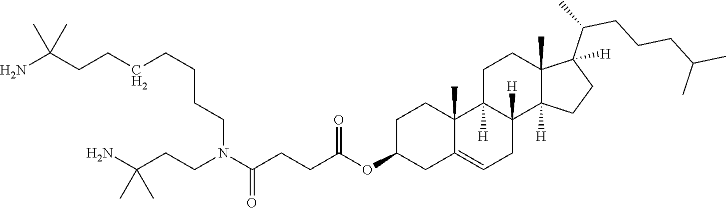 |
| SA165 | 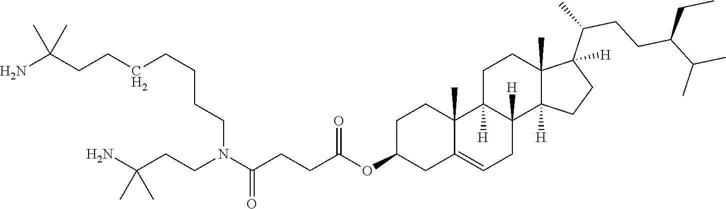 |
| SA166 | 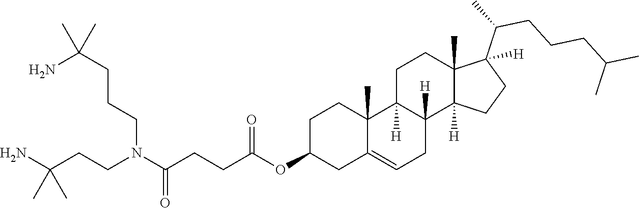 |
| SA167 | 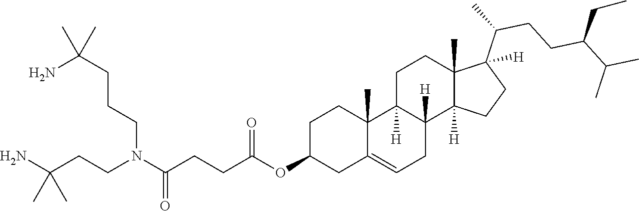 |
| SA168 | 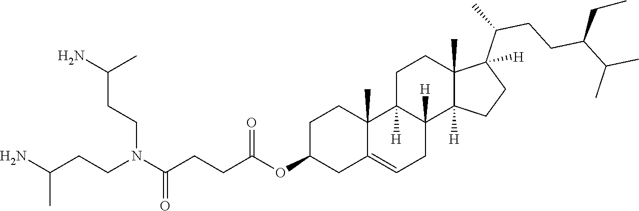 |
| SA169 | 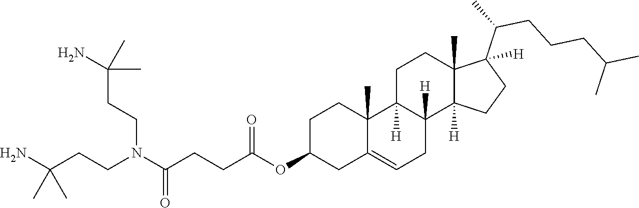 |
| SA170 | 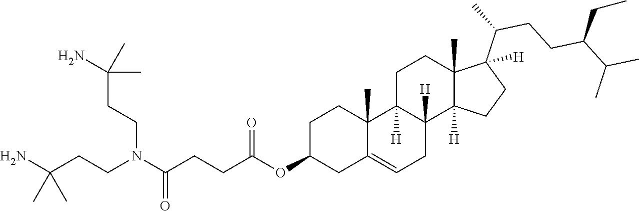 |
| SA171 | 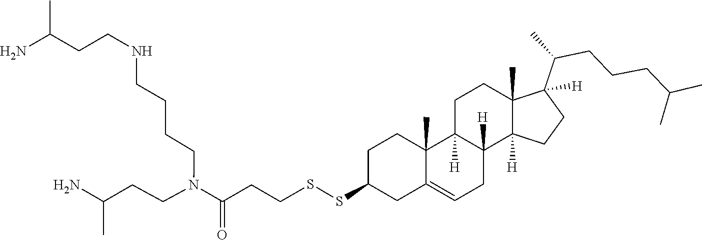 |
| SA172 | 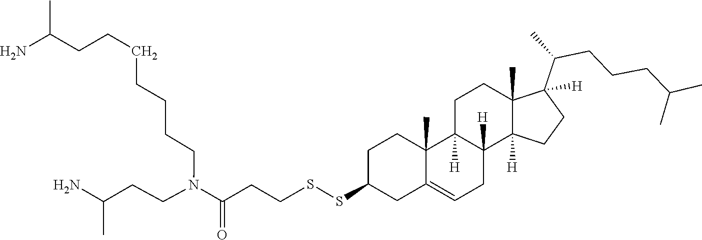 |
| SA173 | 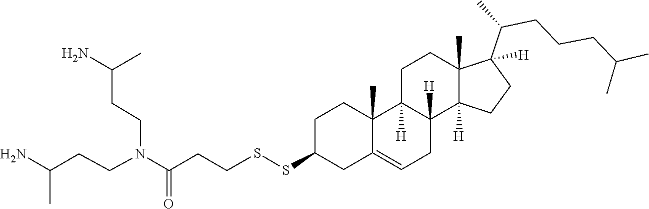 |
| SA174 | 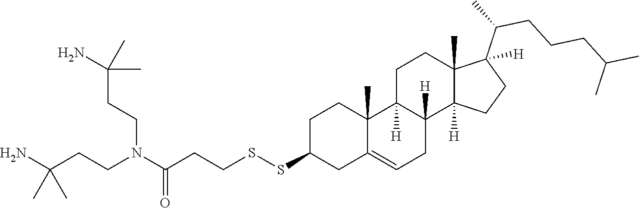 |
| SA175 | 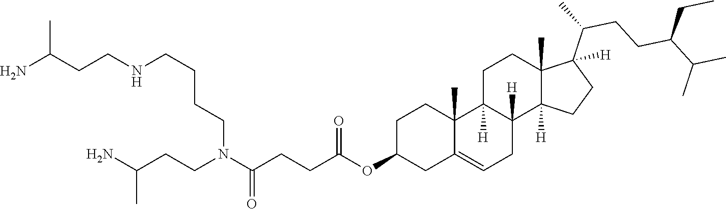 |
| SA176 | 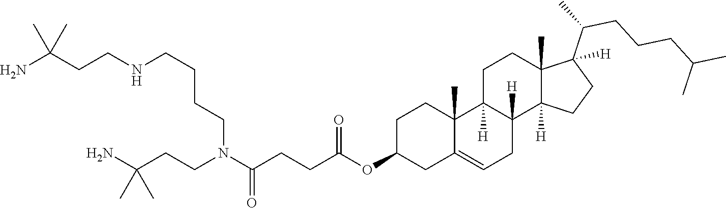 |
| SA177 | 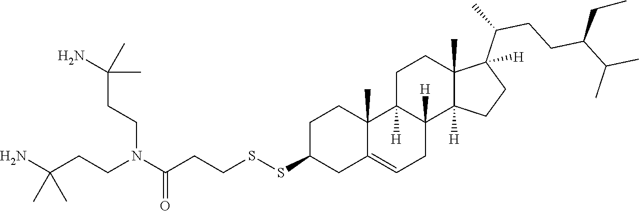 |
| SA178 | 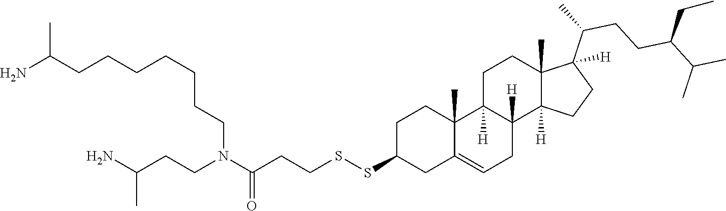 |
| SA179 | 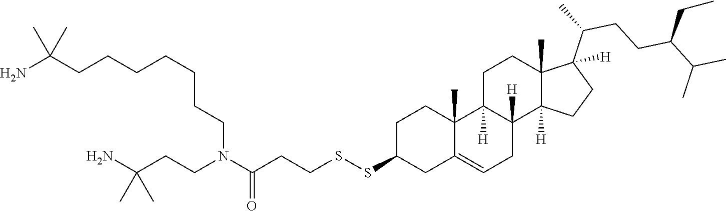 |
| SA180 | 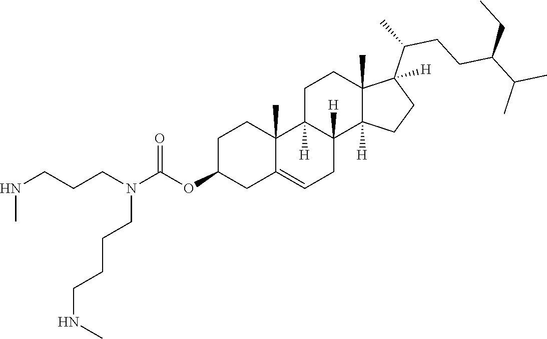 |
| SA181 | 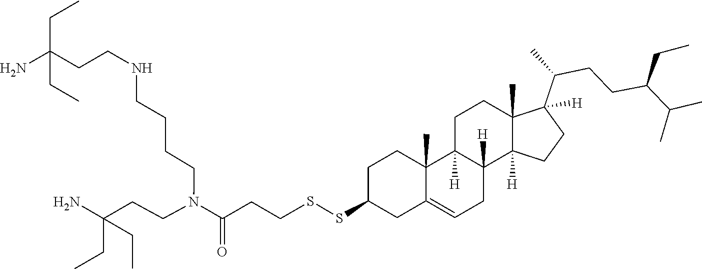 |
| SA182 | 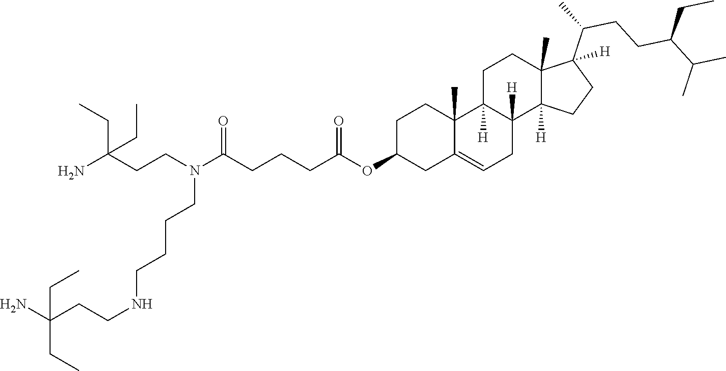 |
| SA183 | 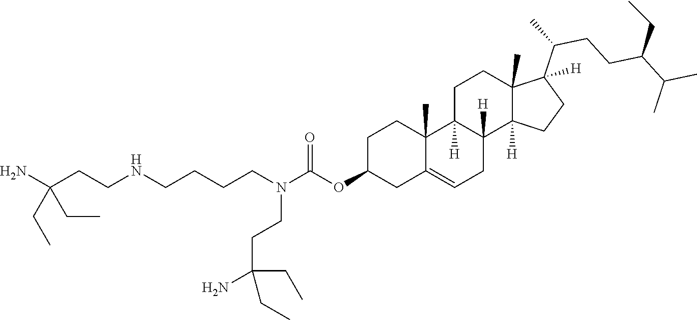 |
| SA184 | 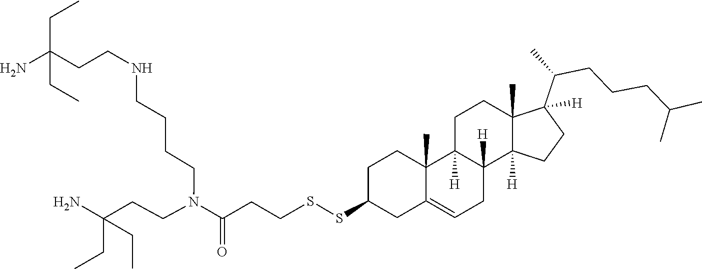 |
| SA185 | 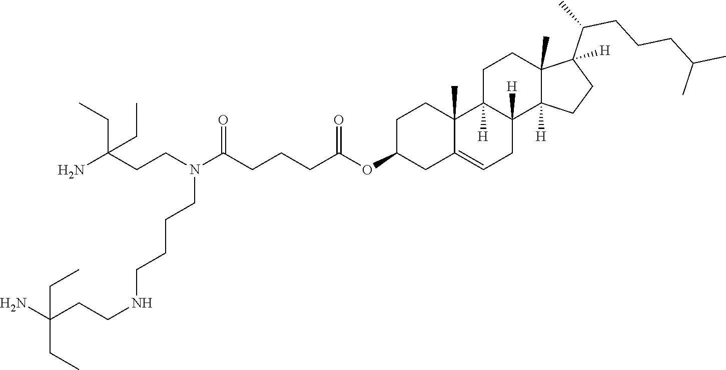 |
| SA186 | 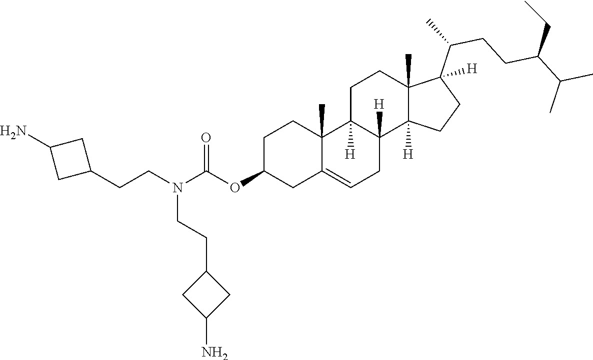 |
| SA187 | 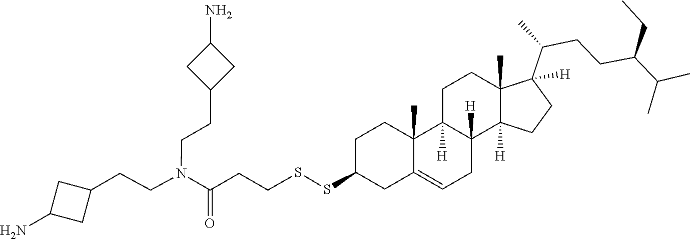 |
| SA188 | 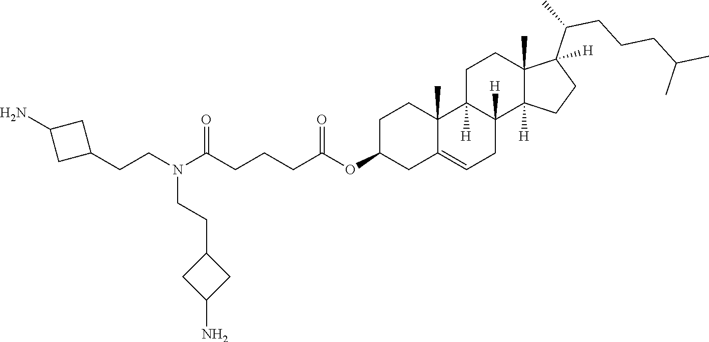 |
| SA189 | 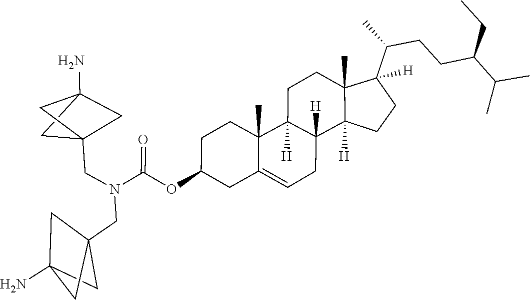 |

or a salt of any of the aforementioned. In some embodiments, the sterol amine of the present invention is selected from the group consisting of: SA186, SA187, SA188 and SA189. In some embodiments, the sterol amine of the present invention is selected from: SA3, SA10, SA18, SA24, SA58, SA78, SA121, SA137, SA138, SA158, and SA183

In some embodiments, the sterol amine of the present invention is a compound having the formula:

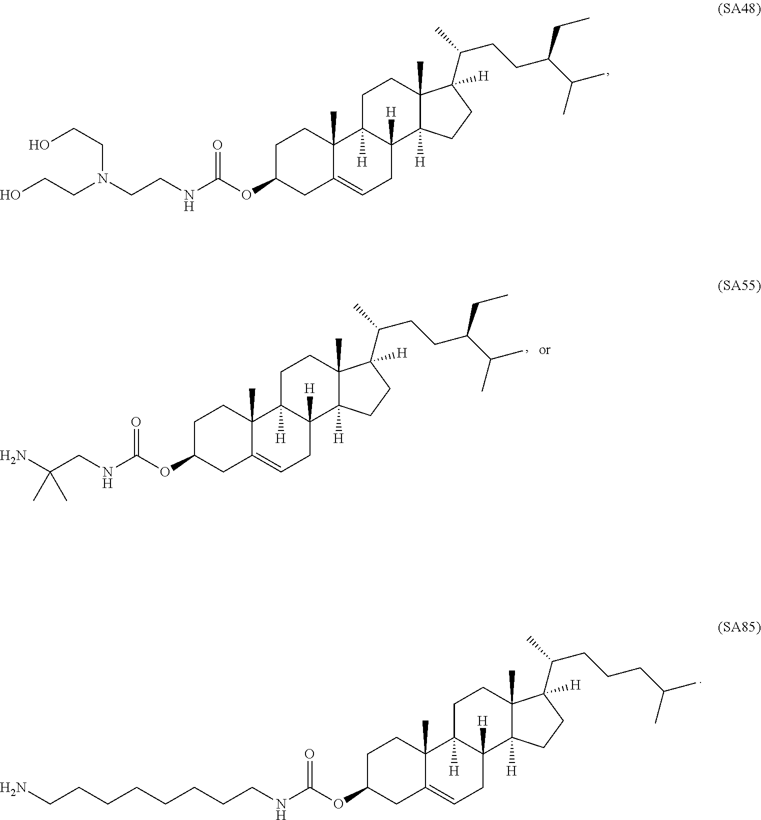

or salt thereof.

In some embodiments, the sterol amine is SA3:

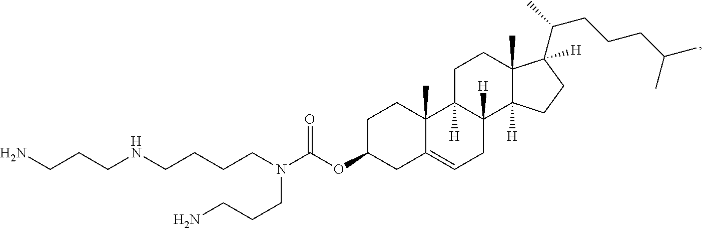

or a salt thereof, which is also referred to as SA3. SA3 can be prepared according to known processes in the art or purchased from a commercial vendor such as Avanti® Polar Lipids, Inc. (SKU 890893).

In some embodiments, the sterol amine is a compound described in WO 2022/032154, the entire contents of which is incorporated herein by reference.

### Lipid Nanoparticle Compositions

The present invention further provides a lipid nanoparticle (LNP) composition comprising a cationic agent (e.g., lipid amine) disclosed herein, such as a lipid amine of Formula A1. In some embodiments, the lipid nanoparticle composition further comprises, in addition to the lipid amine, at least one of an ionizable lipid, a phospholipid, a structural lipid, and a PEG-lipid. In some embodiments, the lipid nanoparticles of the lipid nanoparticle composition are loaded with payload. In some embodiments, the lipid amine is disposed primarily on the outer surface of the lipid nanoparticles of the lipid nanoparticle composition. In some embodiments, the lipid nanoparticle composition has a greater than neutral zeta potential at physiologic pH.

In some embodiments, the lipid nanoparticle composition of the present invention comprises:
(i) an ionizable lipid,
(ii) a phospholipid,
(iii) a structural lipid,
(iv) optionally a PEG-lipid,
(v) optionally a payload for delivery into a cell, and
(vi) a lipid amine as disclosed herein, such as the lipid amine of Formula A1.

The lipid nanoparticle compositions of the invention can further comprise additional components, including but not limited to, helper lipids, stabilizers, salts, buffers, and solvents. The helper lipid is a non-cationic lipid. The helper lipid may comprise at least one fatty acid chain of at least eight carbons and at least one polar headgroup moiety. In some embodiments, the lipid nanoparticle core has a neutral charge at a neutral pH.

In some embodiments, the weight ratio of the lipid amine to payload in the lipid nanoparticle compositions of the invention is about 0.1:1 to about 15:1, about 0.2:1 to about 10:1, about 1:1 to about 10:1, about 1:1 to about 8:1, about 1:1 to about 7:1, about 1:1 to about 6:1, about 1:1 to about 5:1, about 1:1 to about 4:1, or about 1.25:1 to about 3.75:1. In some embodiments, a weight ratio of the lipid amine to payload is about 1.25:1, about 2.5:1, or about 3.75:1. In some embodiments, a molar ratio of the lipid amine to payload is about 0.1:1 to about 20:1, about 1.5:1 to about 10:1, about 1.5:1 to about 9:1, about 1.5:1 to about 8:1, about 1.5:1 to about 7:1, about 1.5:1 to about 6:1, or about 1.5:1 to about 5:1. In some embodiments, a molar ratio of the lipid amine to payload is about 1.5:1, about 2:1, about 3:1, about 4:1, or about 5:1.

In some embodiments, the lipid nanoparticle composition of the invention is characterized as having a zeta potential of about 5 mV to about 20 mV. In some embodiments, the lipid nanoparticle composition has a zeta potential of about 5 mV to about 15 mV. In some embodiments, the lipid nanoparticle composition has a zeta potential of about 5 mV to about 10 mV. Zeta potential measures the surface charge of colloidal dispersions. The magnitude of the zeta potential indicates the degree of electrostatic repulsion between adjacent, similarly charged particles in the dispersion. Zeta potential can be measured on a Wyatt Technologies Mobius Zeta Potential instrument. This instrument characterizes the mobility and zeta potential by the principle of “Massively Parallel Phase Analysis Light Scattering” or MP-PALS. This measurement is more sensitive and less stress inducing than ISO Method 13099-1:2012 which only uses one angle of detection and required higher voltage for operation. In some embodiments, the zeta potential of the herein described empty lipid nanoparticle compositions lipid is measured using an instrument employing the principle of MP-PALS. Zeta potential can be measured on a Malvern Zetasizer (Nano ZS).

In some embodiments, greater than about 80%, greater than about 90%, or greater than about 95% of the lipid amine is on the surface on the lipid nanoparticles of the lipid nanoparticle composition.

In some embodiments, the lipid nanoparticle composition has a polydispersity value of less than about 0.4, less than about 0.3 or less than about 0.2. In some embodiments, the LNP has a polydispersity value of about 0.1 to about 1, about 0.1 to about 0.5 or about 0.1 to about 0.3.

In some embodiments, the lipid nanoparticles of the lipid nanoparticle composition has a mean diameter of about 40 nm to about 150 nm, about 50 nm to about 100 nm, about 60 nm to about 120 nm, about 60 nm to about 100 nm, or about 60 nm to about 80 nm.

In some embodiments, a general polarization of laurdan of the lipid nanoparticles of the lipid nanoparticle composition is greater than or equal to about 0.6. In some embodiments, the LNP has a d-spacing of greater than about 6 nm or greater than about 7 nm.

In some embodiments, at least about 50%, at least about 75%, at least about 90%, at least about 95% of the lipid nanoparticles of the lipid nanoparticle composition have a surface fluidity value of greater than a threshold polarization level.

In some embodiments, the cationic lipid is a modified amino acid, such as a modified arginine, in which an amino acid residue having an amine-containing side chain is appended to a hydrophobic group such as a sterol (e.g., cholesterol or derivative thereof), fatty acid, or similar hydrocarbyl group. At least one amine of the modified amino acid portion has a pKa of 8.0 or greater. At least one amine of the modified amino acid portion is positively charged at physiological pH. The amino acid residue can include but is not limited to arginine, histidine, lysine, tryptophan, ornithine, and 5-hydroxylysine. The amino acid is bonded to the hydrophobic group through a linker.

In some embodiments, the modified amino acid is a modified arginine.

In some embodiments, the cationic agent is a non-lipid cationic agent. Examples of non-lipid cationic agent include e.g., benzalkonium chloride, cetylpyridium chloride, L-lysine monohydrate, or tromethamine.

In some embodiments, the lipid nanoparticle comprises a cationic agent (e.g., a sterol amine) at a molar ratio of 2-15%, 3-10%, 4-10%, 5-10%, 6-10%, 2-3%, 2-4%, 2-5%, 2-6%, 2-7%, 2-8%, 3-4%, 3-5%, 3-6%, 3-7%, 3-8%, 4-5%, 4-6%, 4-7%, 4-8%, 5-6%, 5-7%, 5-8%, 6-7%, 6-8%, 2%, 2.5%, 3%, 3.5%, 4%, 4.5%, 5%, 5.5%, 6%, 6.5%, 7%, 7.5%, 8%, 8.5%, 9%, 9.5%, 10%, at least 2%, at least 3%, at least 4%, at least 5%, at least 6%, at least 7%, at least 8%, less than 15%, less than 14%, less than 13%, less than 12%, less than 11%, or less than 10%. In some embodiments, the lipid nanoparticle comprises a molar ratio of 20-60% ionizable cationic lipid, 5-25% non-cationic lipid, 25-55% sterol, 0.5-15% PEG-modified lipid, and 2-10% cationic agent (e.g., a sterol amine). In some embodiments, the lipid nanoparticle comprises a molar ratio of 40-60% ionizable cationic lipid, 5-15% non-cationic lipid, 30-50% sterol, 0.5-10% PEG-modified lipid, and 3-7% cationic agent. In some embodiments, the lipid nanoparticle comprises a molar ratio of 45-55% ionizable cationic lipid, 7.5-12.5% non-cationic lipid, 35-45% sterol, 0.5-5% PEG-modified lipid, and 4.5-6% cationic agent. In some instances, the cationic agent is SA3 or a salt thereof.

Other exemplary embodiments include (Compound, as used in the table refers to an ionizable amino lipid):

TABLE 3

| Composition (mol %) | Components |
|---|---|
| 48:9.5:35.5:1.5:5.5 | Compound:Phospholipid:Chol: |
| | PEG-lipid:SA3 |
| 47:10:36:1.5:5.5 | Compound:Phospholipid:Chol: |
| | PEG-lipid:SA3 |
| 46:10.5:36.5:1.5:5.5 | Compound:Phospholipid:Chol: |
| | PEG-lipid:SA3 |
| 45:10.5:37.5:1.5:5.5 | Compound:Phospholipid:Chol: |
| | PEG-lipid:SA3 |
| 48:9.5:36:1.5:5 | Compound:Phospholipid:Chol: |
| | PEG-lipid:SA3 |
| 47:10:36.5:1.5:5 | Compound:Phospholipid:Chol: |
| | PEG-lipid:SA3 |
| 46:10.5:37:1.5:5 | Compound:Phospholipid:Chol: |
| | PEG-lipid:SA3 |
| 45:10.5:38:1.5:5 | Compound:Phospholipid:Chol: |
| | PEG-lipid:SA3 |
| 47.6:9.5:36.6:1.4:4.9 | Compound:Phospholipid:Chol: |
| | PEG-lipid:SA3 |
| 45.8:10.5:36.8:1.4:5.5 | Compound:Phospholipid:Chol: |
| | PEG-lipid:SA3 |

TABLE 4

| | mass | MW | Core LNP | PA-LNP | PA-LNP |
|---|---|---|---|---|---|
| Components | (mg) | (g/mol) | mol % | mol % | mass % |
| Ionizable | 11.99 | 710.18 | 50.0% | 47.6% | 51.7% |
| amino Lipid | | | | | |
| DSPC | 2.67 | 790.15 | 10.0% | 9.5% | 11.5% |
| Cholesterol | 5.02 | 386.65 | 38.5% | 36.6% | 21.6% |
| DMG-PEG 2k | 1.27 | 2500 | 1.5% | 1.4% | 5.5% |
| SA3•3HCl | 1.25 | 724 | — | 4.9% | 5.4% |
| HS 15 (excip) | 0.25 | — | — | — | — |
| mRNA | 1 | — | — | — | 4.3% |

HS 15 is macrogol 15 hydroysterarate (Solutol, Kolliphor) having a MW of 960-1900, with average MW of 1430.

TABLE 5

| | mass | MW | Core LNP | PA-LNP | PA-LNP |
|---|---|---|---|---|---|
| Components | (mg) | (g/mol) | mol % | mol % | mass % |
| Ionizable amino | 10.50 | 710.18 | 50.5% | 47.3% | 51.1% |
| Lipid | | | | | |
| DSPC | 2.34 | 790.15 | 10.1% | 9.5% | 11.4% |
| Cholesterol | 4.40 | 386.65 | 38.9% | 36.4% | 21.4% |
| DMG-PEG 2k | 1.06 | 2440 | 0.5% | 1.4% | 5.2% |
| SA3•3HCl | 1.25 | 724 | — | 5.5% | 6.1% |
| mRNA | 1 | — | — | — | 4.9% |

TABLE 6

| | mass | MW | Core LNP | PA-LNP | PA-LNP |
|---|---|---|---|---|---|
| Components | (mg) | (g/mol) | mol % | mol % | mass % |
| Ionizable | 10.18 | 710.18 | 49.0% | 45.8% | 49.6% |
| amino Lipid | | | | | |
| DSPC | 2.58 | 790.15 | 11.2% | 10.5% | 12.6% |
| Cholesterol | 4.45 | 386.65 | 39.3% | 36.8% | 21.7% |
| DMG-PEG 2k | 1.08 | 2500 | 0.5% | 1.4% | 5.3% |
| SA3•3HCl | 1.25 | 724 | — | 5.5% | 6.1% |
| HS 15 (excip) | | | | | |
| mRNA | 1.00 | — | — | — | 4.7% |

In some embodiments, a weight ratio of the cationic agent to polynucleotide is about 0.1:1 to about 15:1. In some embodiments, a weight ratio of the cationic agent to polynucleotide is about 0.2:1 to about 10:1. In some embodiments, a weight ratio of the cationic agent to polynucleotide is about 1:1 to about 10:1. In some embodiments, a weight ratio of the cationic agent to polynucleotide is about 1:1 to about 8:1. In some embodiments, a weight ratio of the cationic agent to polynucleotide is about 1:1 to about 7:1. In some embodiments, a weight ratio of the cationic agent to polynucleotide is about 1:1 to about 6:1. In some embodiments, a weight ratio of the cationic agent to polynucleotide is about 1:1 to about 5:1. In some embodiments, a weight ratio of the cationic agent to polynucleotide is about 1:1 to about 4:1. In some embodiments, a weight ratio of the cationic agent to polynucleotide is about 1.25:1 to about 3.75:1. In some embodiments, a weight ratio of the cationic agent to polynucleotide is about 1.25:1. In some embodiments, a weight ratio of the cationic agent to polynucleotide is about 2.5:1. In some embodiments, a weight ratio of the cationic agent to polynucleotide is about 3.75:1.

In some embodiments, a molar ratio of the cationic agent to polynucleotide is about 0.1:1 to about 20:1. In some embodiments, a molar ratio of the cationic agent to polynucleotide is about 1.5:1 to about 10:1. In some embodiments, a molar ratio of the cationic agent to polynucleotide is about 1.5:1 to about 9:1. In some embodiments, a molar ratio of the cationic agent to polynucleotide is about 1.5:1 to about 8:1. In some embodiments, a molar ratio of the cationic agent to polynucleotide is about 1.5:1 to about 7:1. In some embodiments, a molar ratio of the cationic agent to polynucleotide is about 1.5:1 to about 6:1. In some embodiments, a molar ratio of the cationic agent to polynucleotide is about 1.5:1 to about 5:1. In some embodiments, a molar ratio of the cationic agent to polynucleotide is about 1.5:1. In some embodiments, a molar ratio of the cationic agent to polynucleotide is about 2:1. In some embodiments, a molar ratio of the cationic agent to polynucleotide is about 3:1. In some embodiments, a molar ratio of the cationic agent to polynucleotide is about 4:1. In some embodiments, a molar ratio of the cationic agent to polynucleotide is about 5:1.

In some embodiments, the nanoparticle has a zeta potential of about 5 mV to about 20 mV. In some embodiments, the nanoparticle has a zeta potential of about 5 mV to about 20 mV. In some embodiments, the nanoparticle has a zeta potential of about 5 mV to about 15 mV. In some embodiments, the nanoparticle has a zeta potential of about 5 mV to about 10 mV.

In some embodiments, the lipid nanoparticle core has a neutral charge at a neutral pH.

In some embodiments, greater than about 80% of the cationic agent is on the surface on the nanoparticle. In some embodiments, greater than about 90% of the cationic agent is on the surface on the nanoparticle. In some embodiments, greater than about 95% of the cationic agent is on the surface on the nanoparticle.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

### Ionizable Lipid

As used herein, the term “ionizable lipid” has its ordinary meaning in the art and may refer to a lipid comprising one or more charged moieties. In some embodiments, an ionizable lipid may be positively charged or negatively charged. For instance, an ionizable lipid may be positively charged at lower pHs, in which case it could be referred to as “cationic lipid.” For example, an ionizable lipid may be protonated and therefore positively charged at physiological pH, in which case it could be referred to as “cationic lipid.” An ionizable lipid may be a cationic lipid, and vice versa. In certain embodiments, an ionizable lipid molecule may comprise an amine group, and can be referred to as an ionizable amino lipids.

As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

The terms “charged” or “charged moiety” do not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid. In one embodiment, the ionizable amino lipid may have a positively charged hydrophilic head and a hydrophobic tail that are connected via a linker structure.

In some embodiments, the nanoparticle described herein comprises about 30 mol % to about 60 mol % of ionizable lipid. In some embodiments, the nanoparticle comprises about 40 mol % to about 50 mol % of ionizable lipid. In some embodiments, the nanoparticle comprises about 35 mol % to about 55 mol % of ionizable lipid. In some embodiments, the nanoparticle comprises about 45 mol % to about 50 mol % of ionizable lipid.

A lipid nanoparticle composition of the invention may include one or more ionizable (e.g., ionizable amino) lipids (e.g., lipids that may have a positive or partial positive charge at physiological pH).

Ionizable lipids may be selected from the non-limiting group consisting of 3-(didodecylamino)-N1,N1,4-tridodecyl-1-piperazineethanamine (KL10), Ni-[2-(didodecylamino)ethyl]N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl-4-(dimethylamino)butanoate (DLin-MC3-DMA), 2,2-dilinoleyl-4-(2 dimethylaminoethyl)-[1,3]-dioxolane (DLin-KC2-DMA), 1,2-dioleyloxy-N,N-dimethylaminopropane (DODMA), 2-({8 [(3β)-cholest-5-en-3-yloxy]octyl}oxy) N,N dimethyl-3-[(9Z,12Z)-octadeca-9,12-dien-1-yloxy]propan-1-amine (Octyl-CLinDMA), (2R)-2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S) 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable lipid may also be a lipid including a cyclic amine group.

Examples of ionizable amino lipids can be found in, e.g., International PCT Application Publication Nos. WO 2017/049245, published Mar. 23, 2017; WO 2017/112865, published Jun. 29, 2017; WO 2018/170306, published Sep. 20, 2018; WO 2018/232120, published Dec. 20, 2018; WO 2020/061367, published Mar. 26, 2020; WO 2021/055835, published Mar. 25, 2021; WO 2021/055833, published Mar. 25, 2021; WO 2021/055849, published Mar. 25, 2021; and WO 2022/204288, published Sep. 29, 2022, the entire contents of each of which is incorporated herein by reference.

Ionizable lipids can also be the compounds disclosed in International Publication No. WO 2017/075531 A1, hereby incorporated by reference in its entirety. For example, the ionizable amino lipids include, but not limited to:

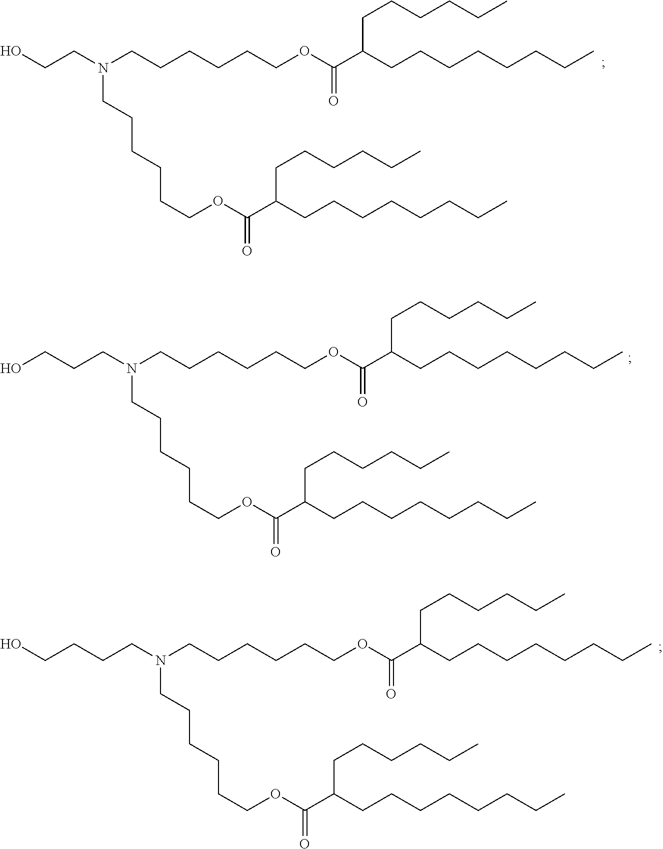 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC]

and any combination thereof.

Ionizable lipids can also be the compounds disclosed in International Publication No. WO 2015/199952 A1, hereby incorporated by reference in its entirety. For example, the ionizable amino lipids include, but not limited to:

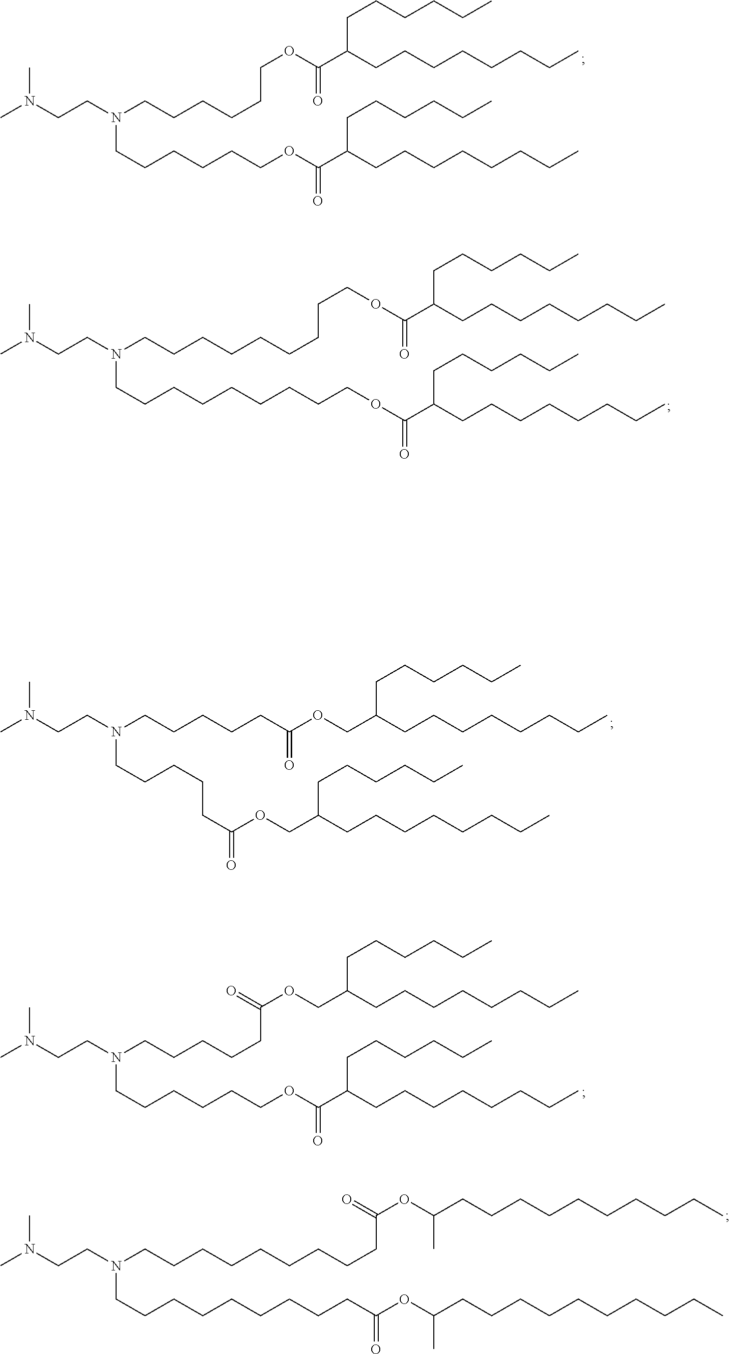

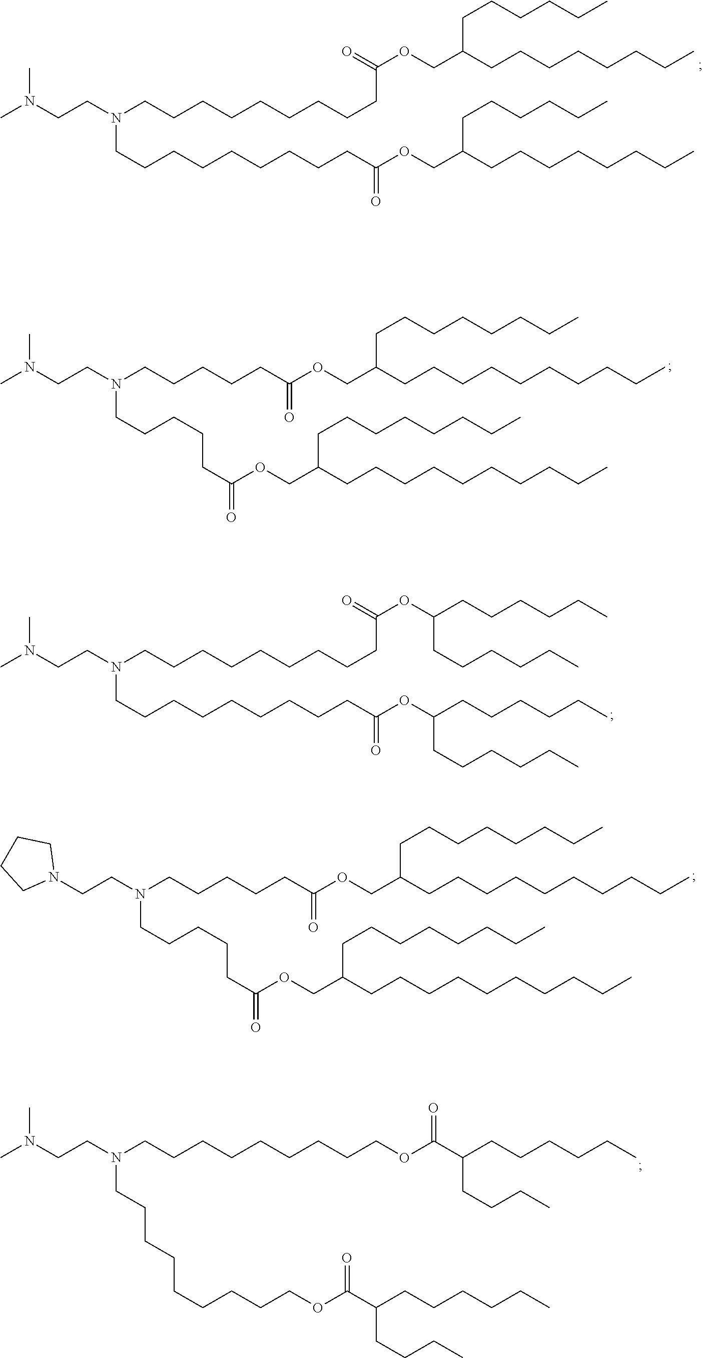

and any combination thereof.

In one embodiment, the ionizable lipid may be selected from, but not limited to, an ionizable lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and S20130225836; the contents of each of which are herein incorporated by reference in their entirety.

In another embodiment, the ionizable lipid may be selected from, but not limited to, formula A described in International Publication Nos. WO2013116126 or US20130225836; the contents of each of which is herein incorporated by reference in their entirety. In yet another embodiment, the ionizable lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115, formula I of US Patent Publication No US20130123338; each of which is herein incorporated by reference in their entirety.

As a non-limiting example, a cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16, 19-dien-8-amine, (13Z,16Z)—N,N-dimethyldocosa-13,16-dien-5-amine, (12Z,15Z)—N,N dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2 undecylcyclopropyl]tetradecan-5-amine, N,N-dimethyl-3-{7-[(1S,2R)-2-octylcyclopropyl]heptyl}dodecan-1-amine, 1-[(1R,2S)-2-hepty lcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethylIpyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)- docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-octylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

Additional examples of ionizable lipids include the following:

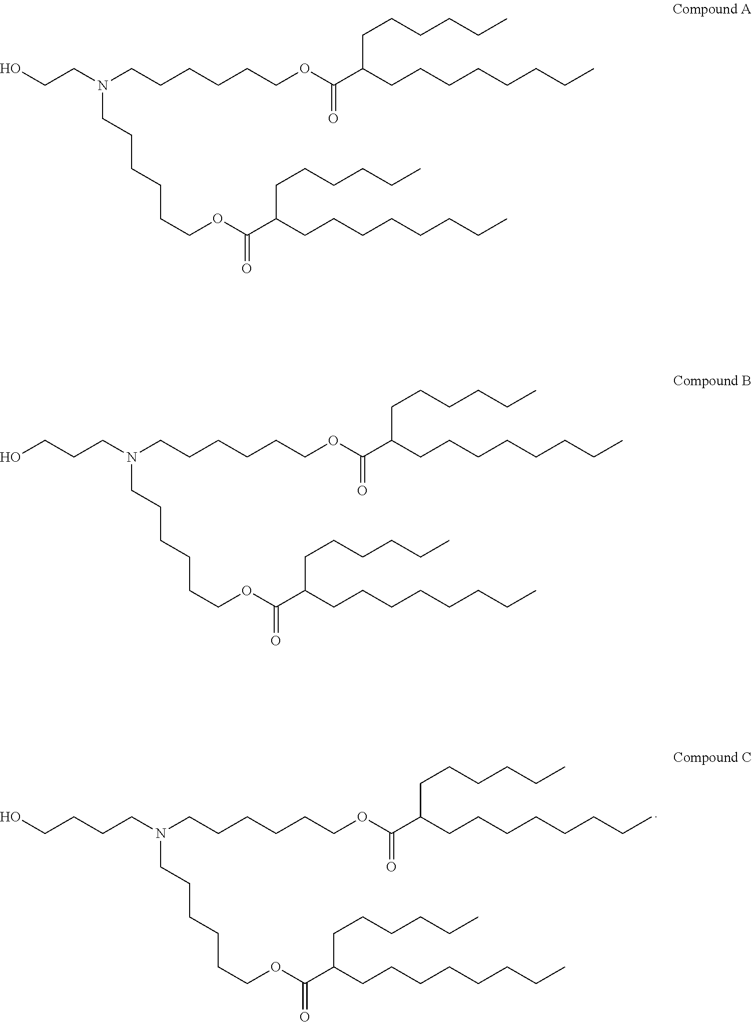 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; "."]

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety. In another embodiment, the lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

In some embodiments, the ionizable lipid may be a compound of Formula (I):

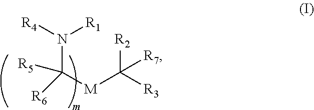

or a salt or isomer thereof, wherein:
R¹ is selected from the group consisting of H, C₅₋₃₀ alkyl, C₅₋₃₀ alkenyl, —R*YR″, —YR″, —(CH₂)ₙ(NR⁴)R″M′R′, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle, wherein the carbocycle is optionally substituted with C₆ cycloalkyl or C₅₋alkyl;
R⁴ is selected from the group consisting of a C₃₋₆ carbocycle, —(CH₂)ₙQ, —(CH₂)ₙCHQR, —CHQR, —CQ(R)₂, —CH(CH₂Q)₂, and unsubstituted C₁₋₆ alkyl, wherein the C₃₋₆ carbocycle is optionally substituted with —OH or —OMe;
each Q is independently selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —(CH₂)nOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O) N(R)OR, and —C(R)N(R)₂C(O)OR;
or Q is selected from:

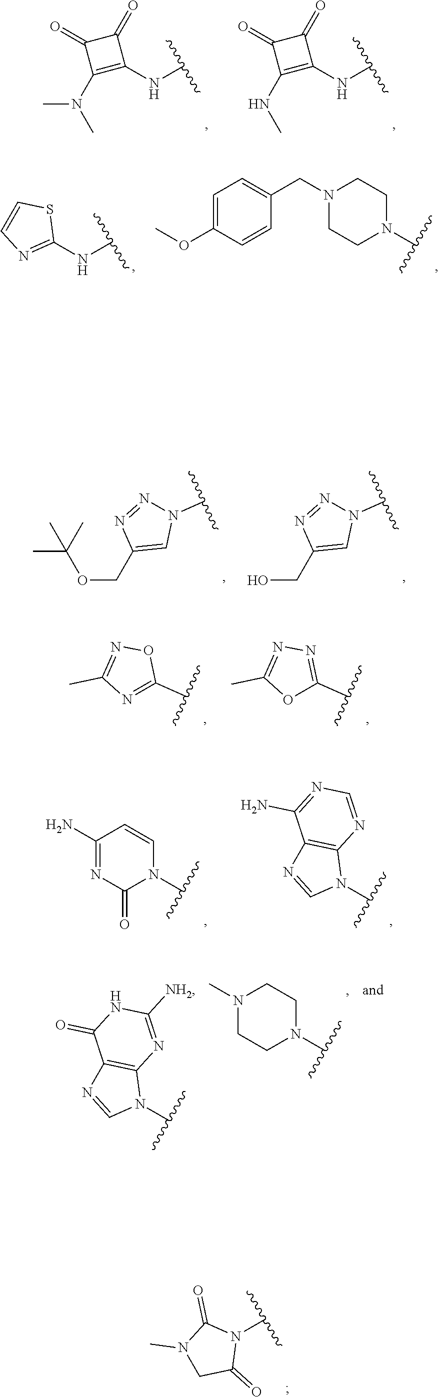

each n is independently selected from 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H, wherein C₁₋₃ alkyl is optionally substituted with —OH, —C(O)OH, —OMe, —O-benzyl,
each R′ is independently selected from the group consisting of C₁₋₁₈ alkyl, C₂₋₁₈ alkenyl, —R*YR″, —YR″, and H, wherein C₁₋₁₈ alkyl is optionally substituted with —OMe;
each R″ is independently selected from the group consisting of H, C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, the ionizable lipid may be a compound of Formula (I):

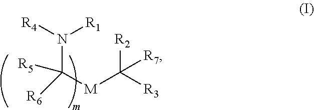

or a salt or isomer thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, —CN, —NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)·CHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR⁹)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (=O), OH, amino, mono- or di-alkylamino, and C₁₋₃ alkyl, and each n is independently selected from 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, —CN, —NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R₁ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, another subset of compounds of Formula (I) includes those in which
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)nOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R⁷ is selected from the group consisting of C₁₋₃ alkyl, C₂₋₃ alkenyl, and H;
R⁸ is selected from the group consisting of C₃₋₆ carbocycle and heterocycle;
R⁹ is selected from the group consisting of H, —CN, —NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, another subset of compounds of Formula (I) includes those in which
R¹ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of a C₃₋₆ carbocycle, —(CH₂)ₙQ, —(CH₂)·CHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, —CN, —NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R¹ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R¹ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, another subset of compounds of Formula (I) includes those in which
R¹ is selected from the group consisting of C₅₋₃₀ alkyl, C₅₋₂₀ alkenyl, —R*YR″, —YR″, and —R″M′R′;
R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of —(CH₂)ₙQ, —(CH₂)·CHQR, —CHQR, and —CQ(R)₂, where Q is —N(R)₂, and n is selected from 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R⁷ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
   or salts or isomers thereof.

In some embodiments, a subset of compounds of Formula (I) includes those of Formula (IA):

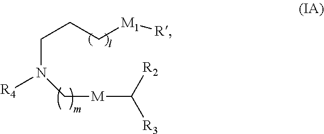

or a salt or isomer thereof, wherein l is selected from 1, 2, 3, 4, and 5; m is selected from 5, 6, 7, 8, and 9; M₁ is a bond or M′; R⁴ is unsubstituted C₁₋₃ alkyl, or —(CH₂)ₙQ, in which Q is —OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

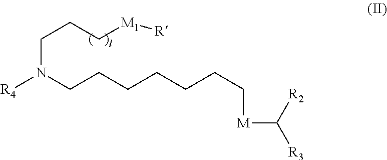

or a salt or isomer thereof, wherein l is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

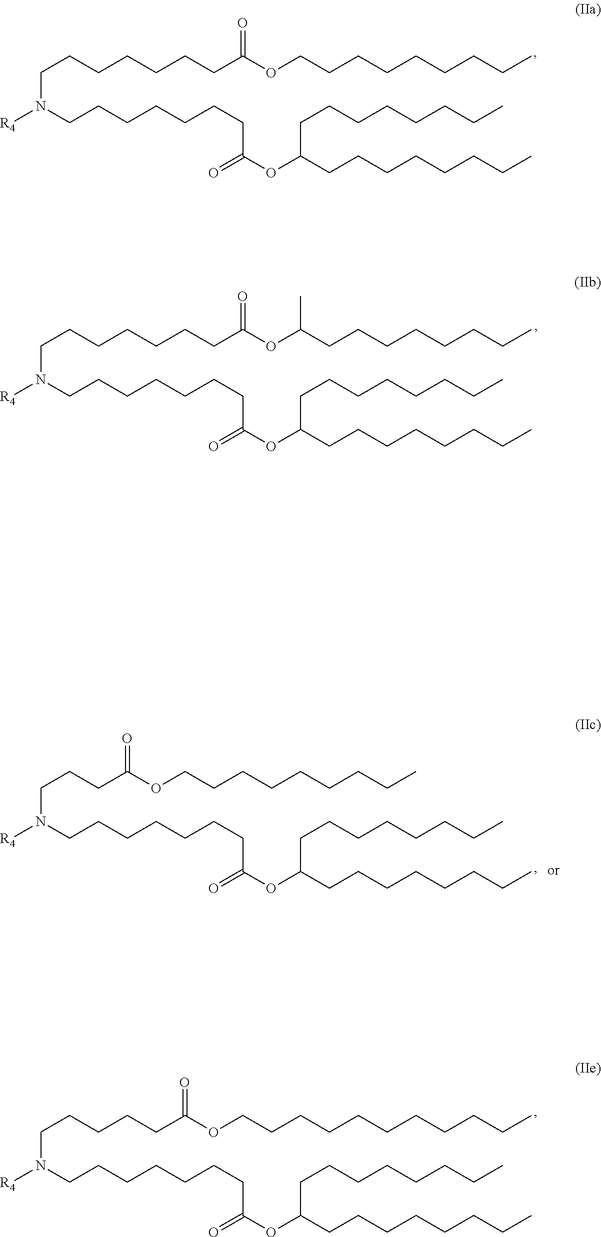

or a salt or isomer thereof, wherein R₄ is as described herein.

In some embodiments, a subset of compounds of Formula (I) includes those of Formula (IId):

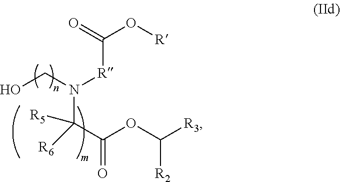

or a salt or isomer thereof, wherein n is 2, 3, or 4; and m, R′, R″, and R₂ through R₆ are as described herein. For example, each of R₂ and R₃ may be independently selected from the group consisting of C₅₋₁₄alkyl and C₅₋₁₄ alkenyl.

In some embodiments, the compound of Formula (I) is selected from the group consisting of:

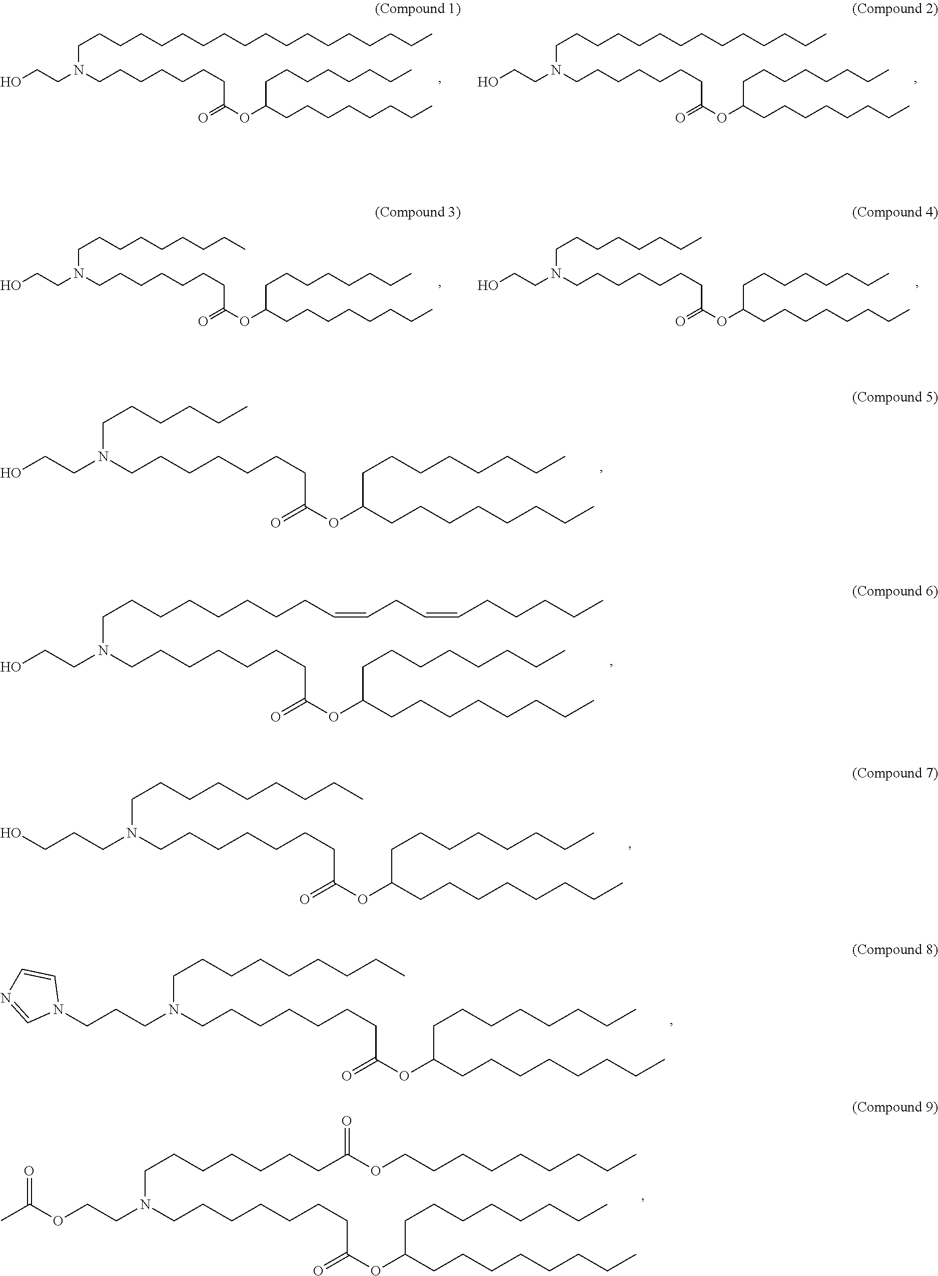

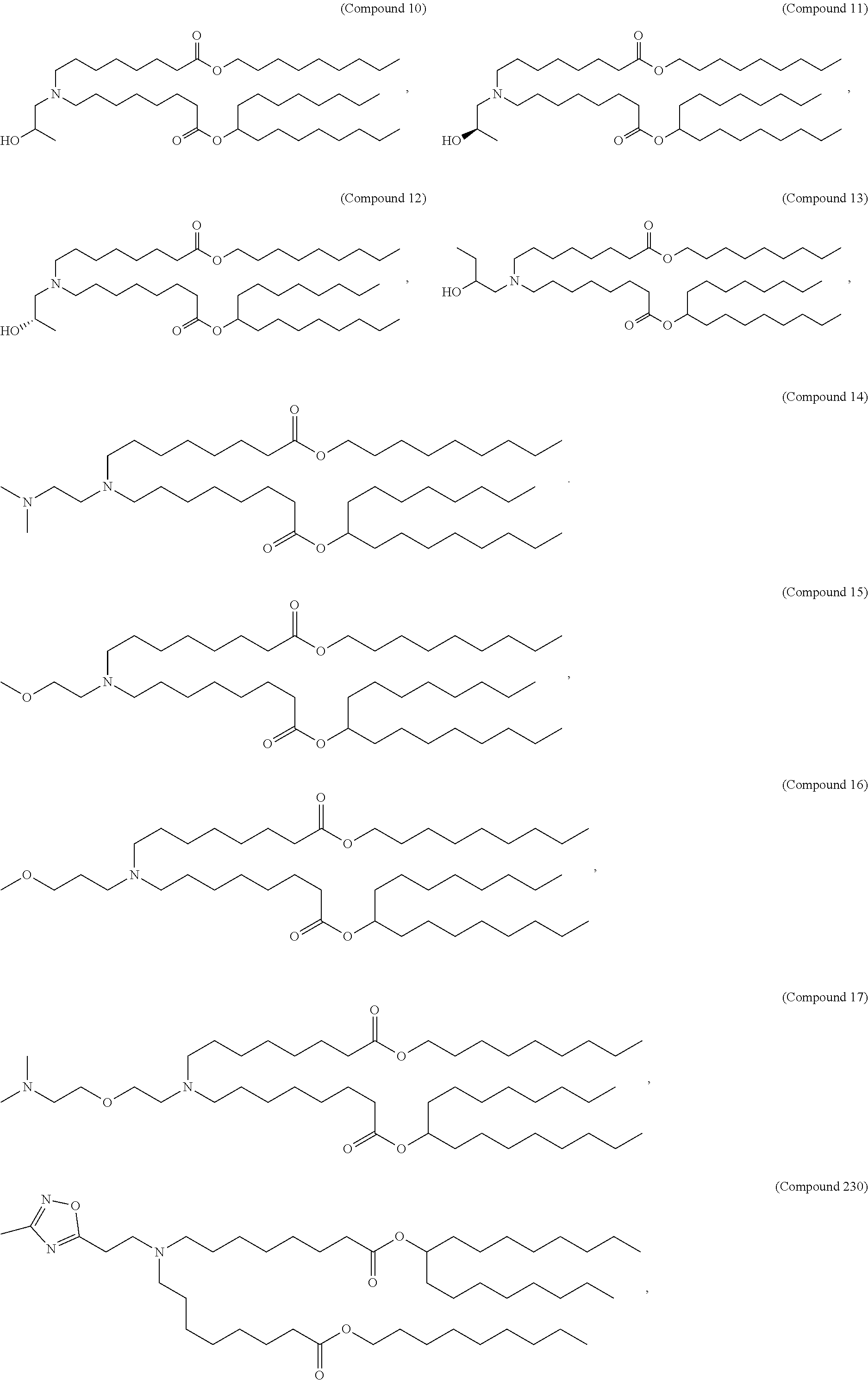

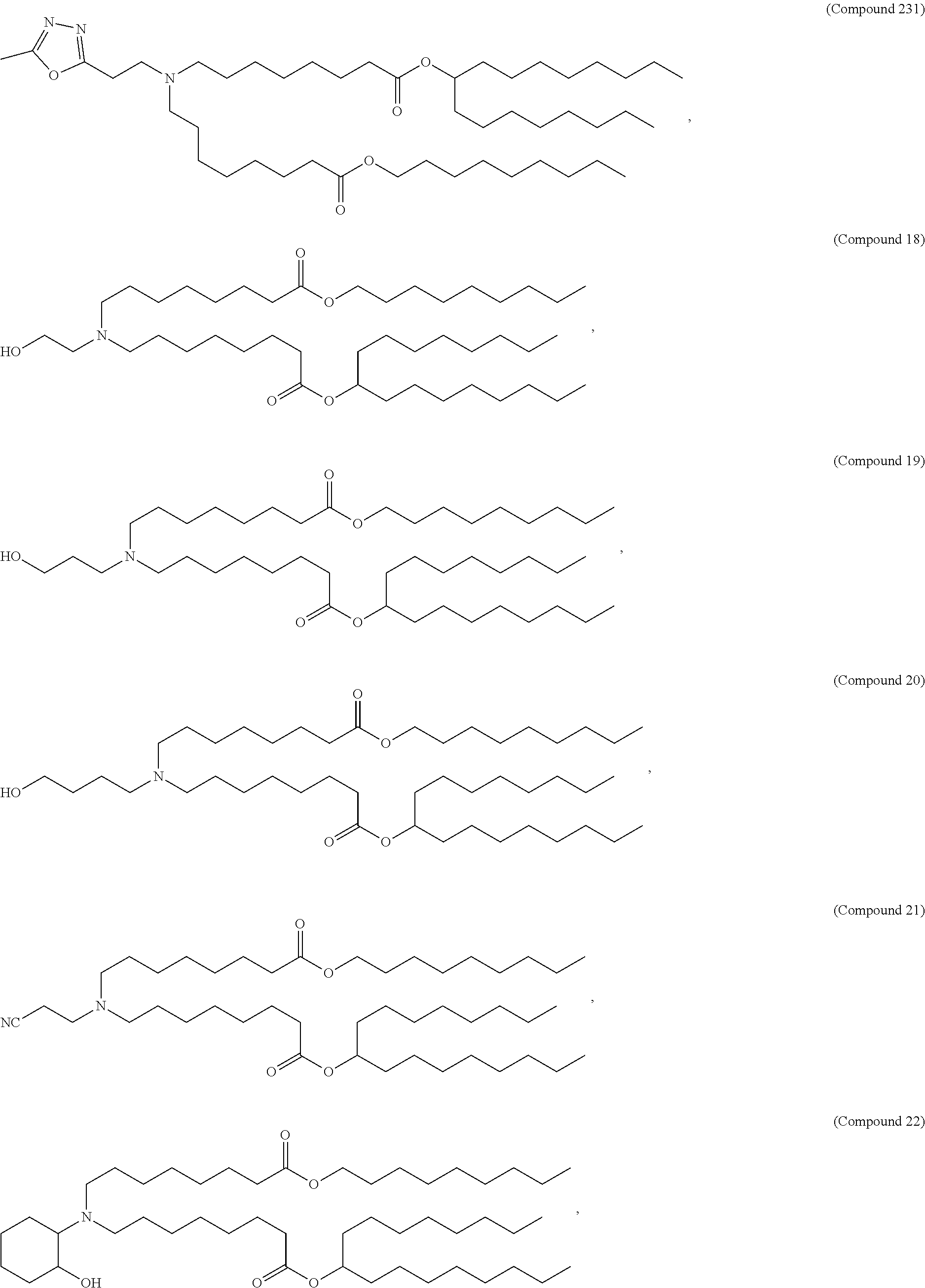 [SMILES: CCCCCCCCCOC(=O)CCCCCCCN(CCCCCCCC(=O)OC(CCCCCCCC)CCCCCCCC)CCc1nnc(C)o1 ; CCCCCCCCCOC(=O)CCCCCCCN(CCO)CCCCCCCC(=O)OC(CCCCCCCC)CCCCCCCC ; CCCCCCCCCOC(=O)CCCCCCCN(CCCO)CCCCCCCC(=O)OC(CCCCCCCC)CCCCCCCC ; CCCCCCCCCOC(=O)CCCCCCCN(CCCCO)CCCCCCCC(=O)OC(CCCCCCCC)CCCCCCCC ; CCCCCCCCCOC(=O)CCCCCCCN(CCC#N)CCCCCCCC(=O)OC(CCCCCCCC)CCCCCCCC ; CCCCCCCCCOC(=O)CCCCCCCN(CCCCCCCC(=O)OC(CCCCCCCC)CCCCCCCC)C1CCCCC1O]

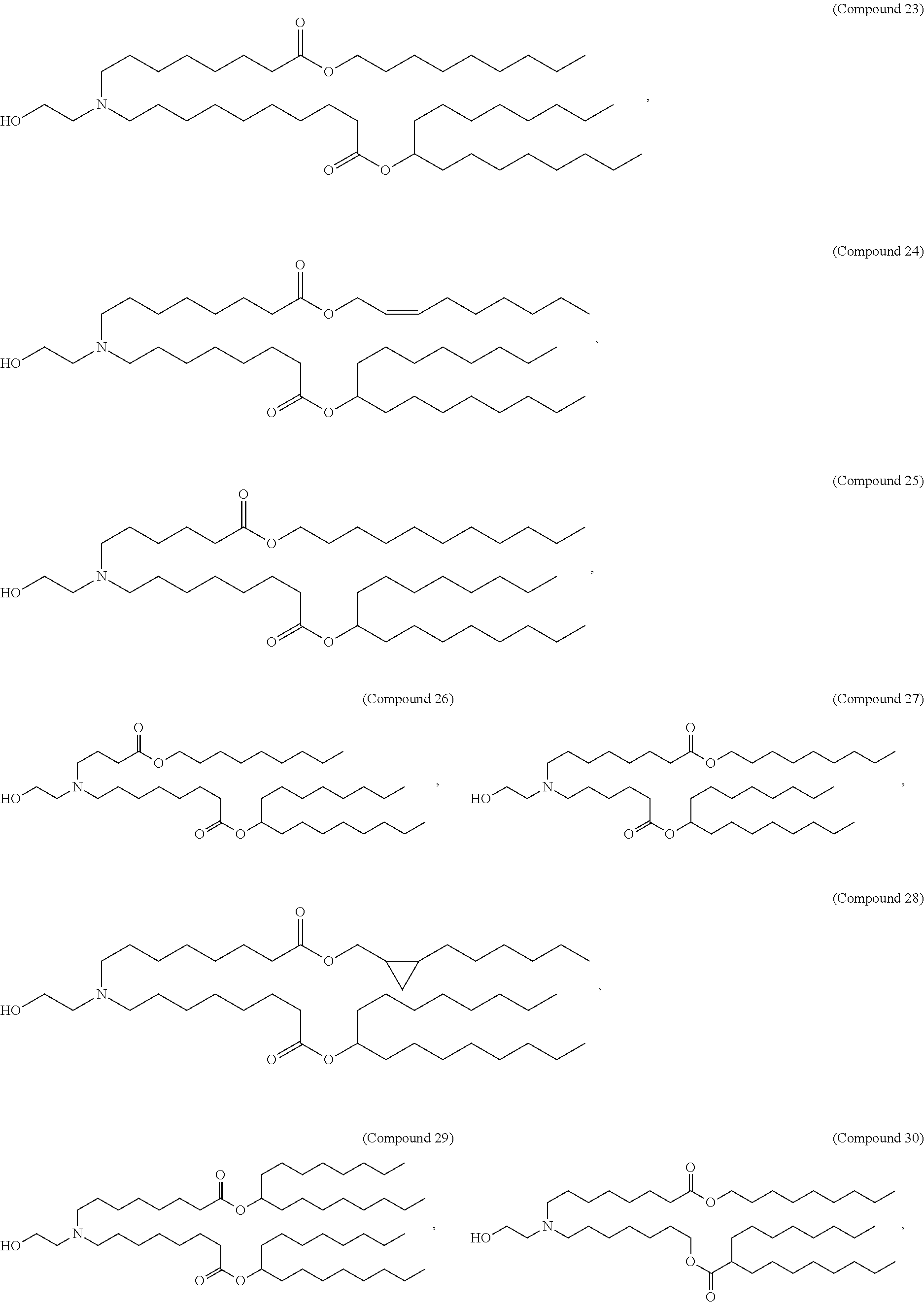

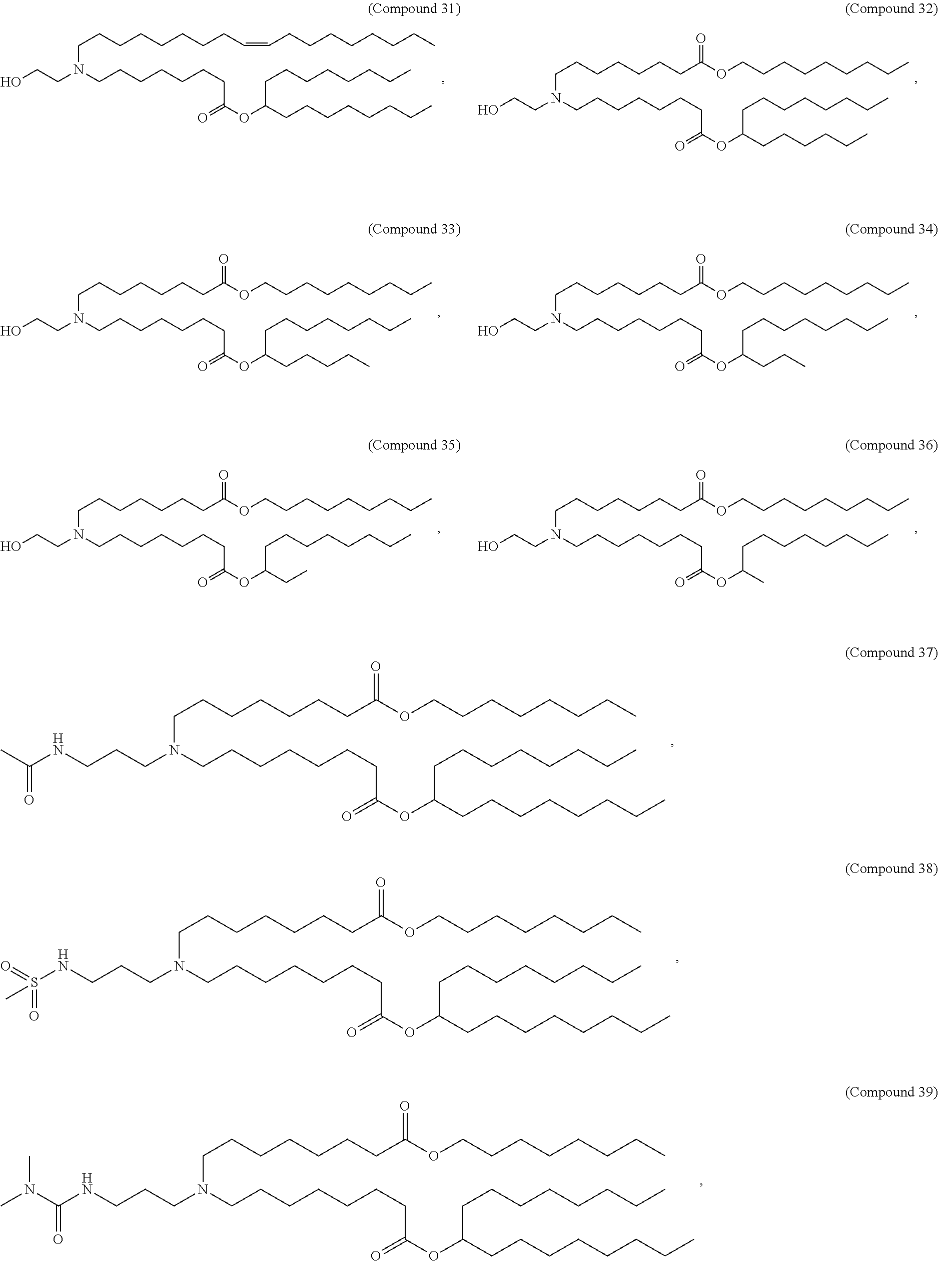

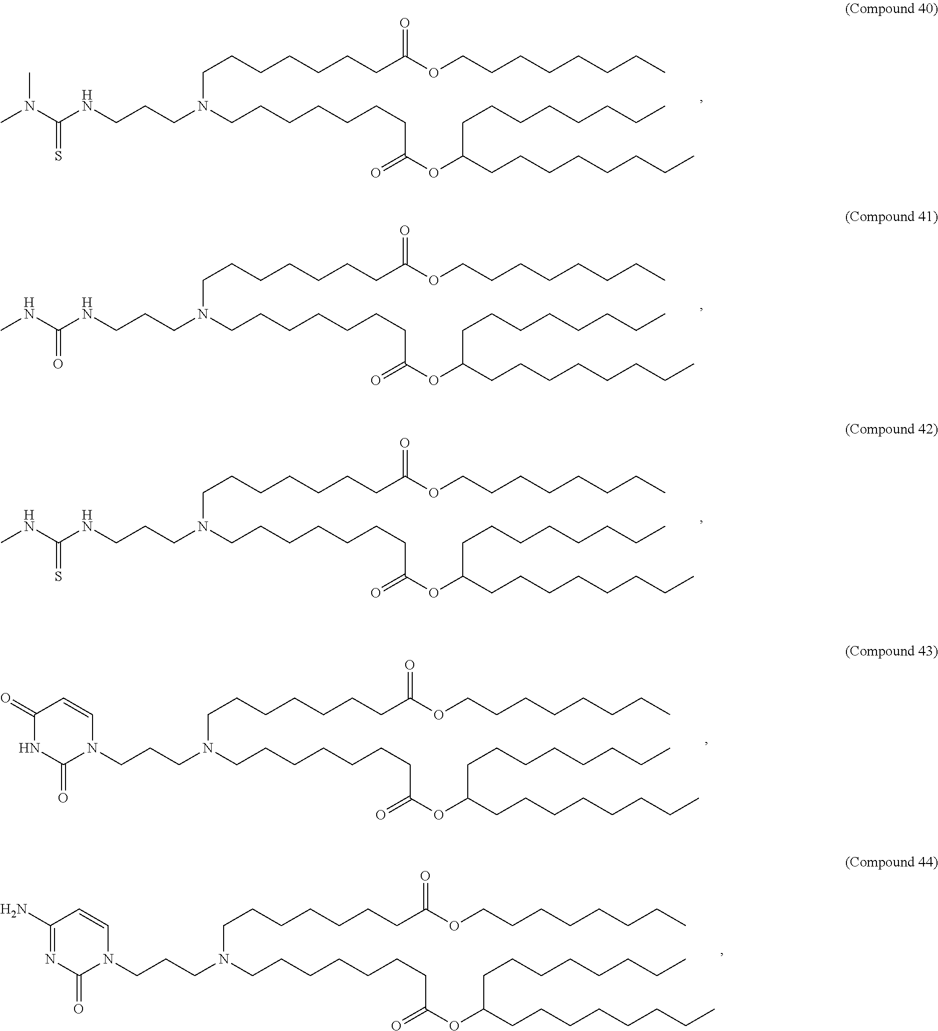

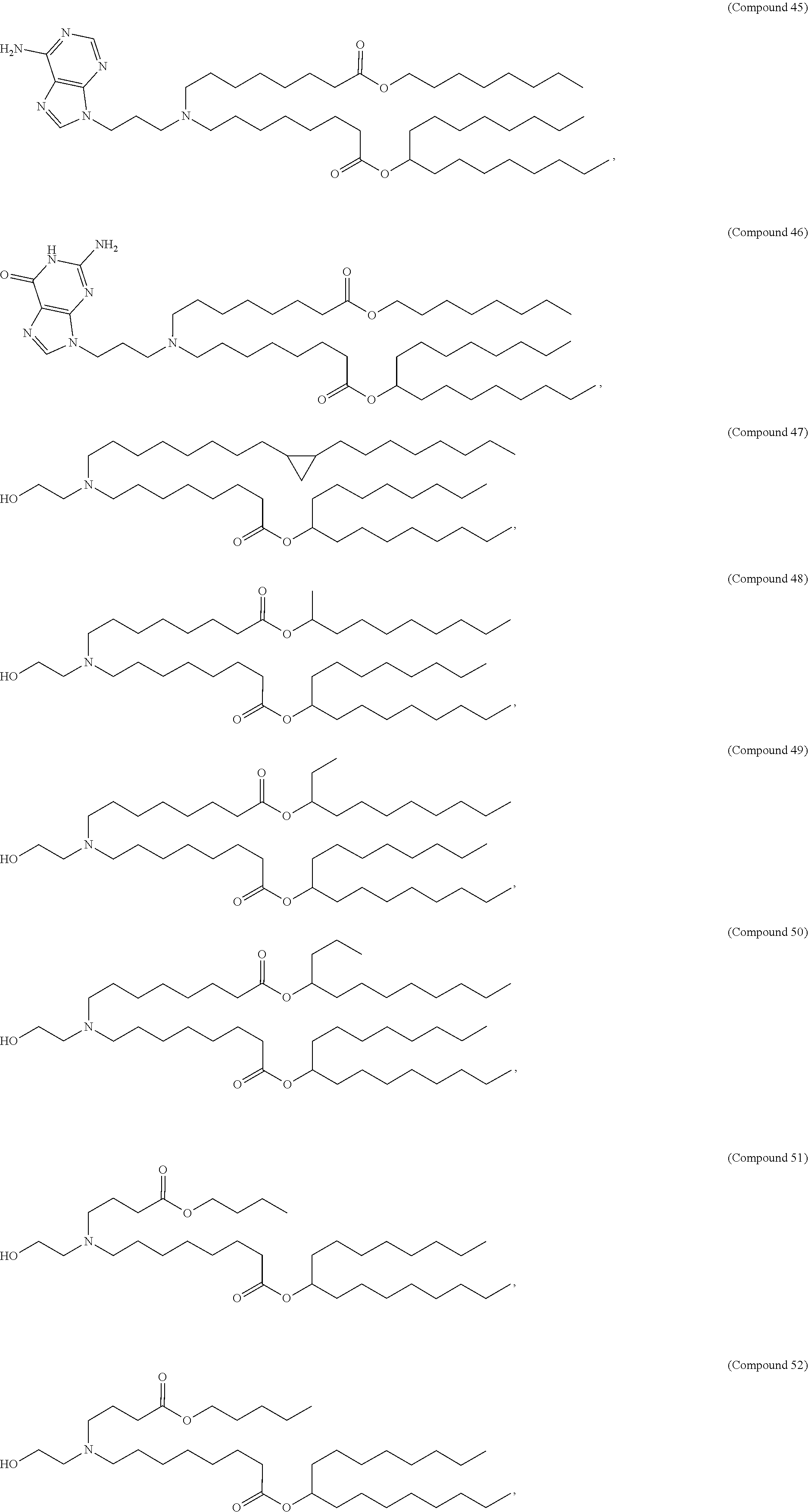

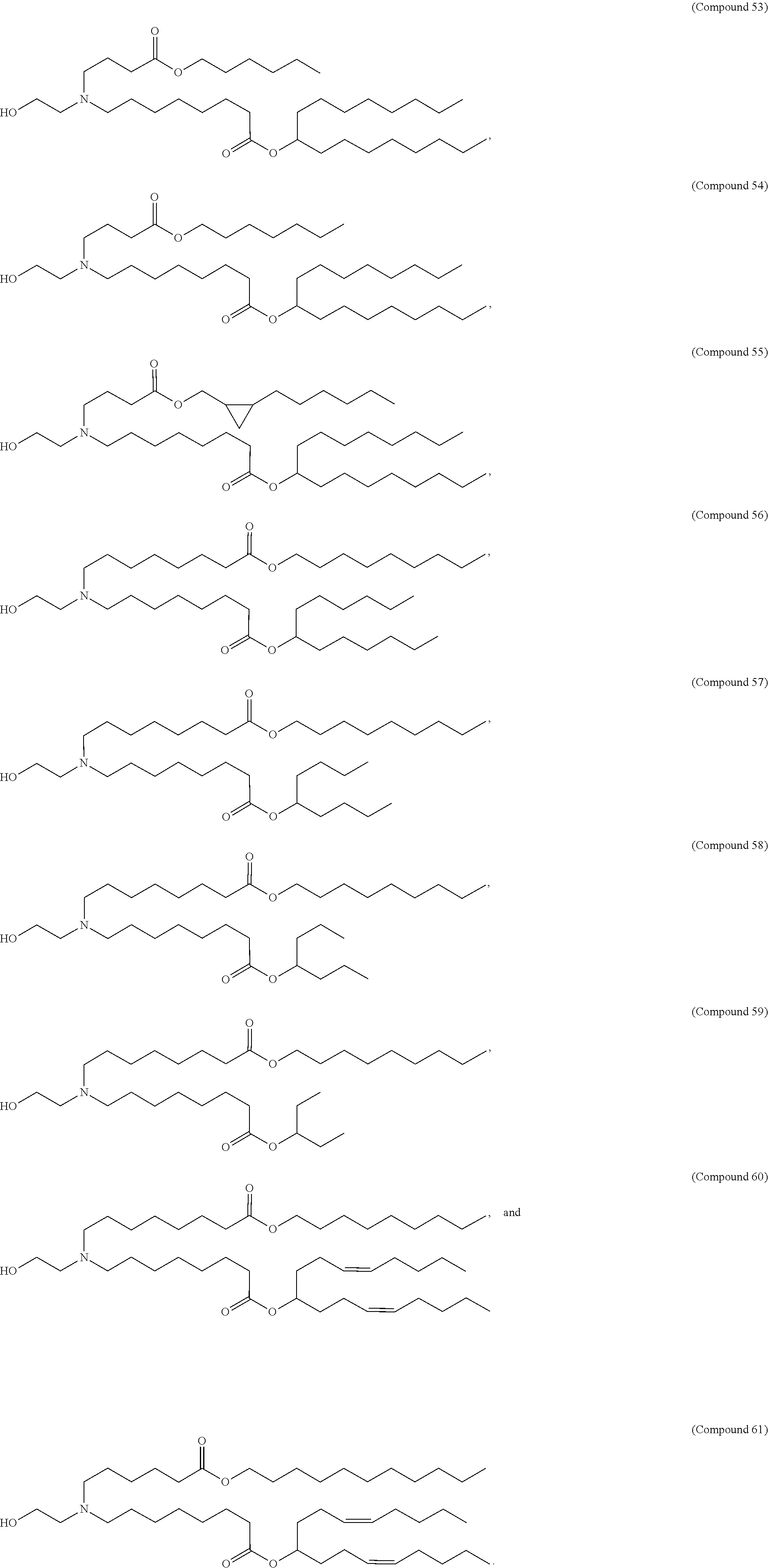

In further embodiments, the compound of Formula (I) is selected from the group consisting of:

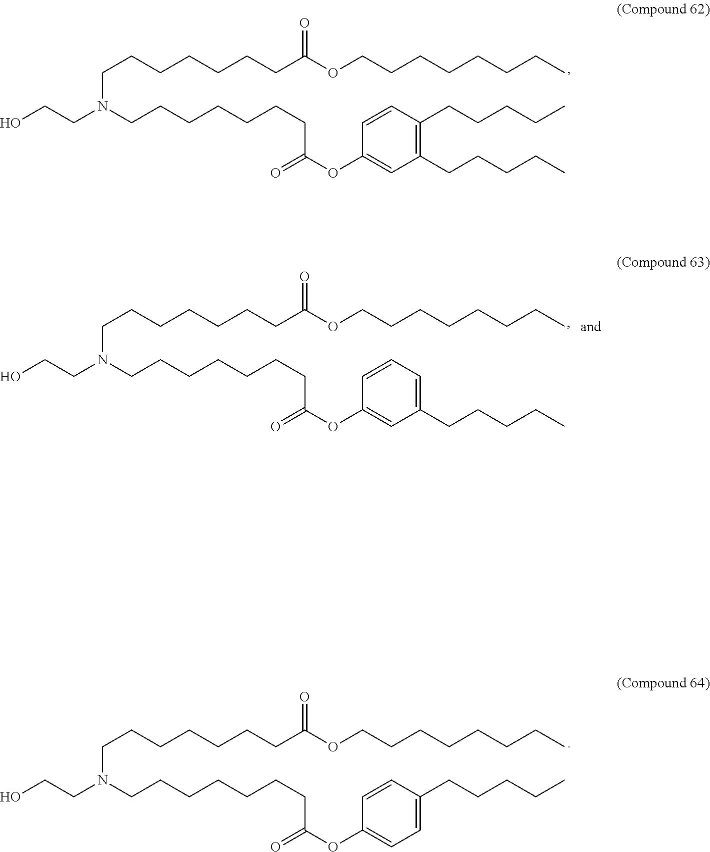

In some embodiments, the compound of Formula (I) is selected from the group consisting of:

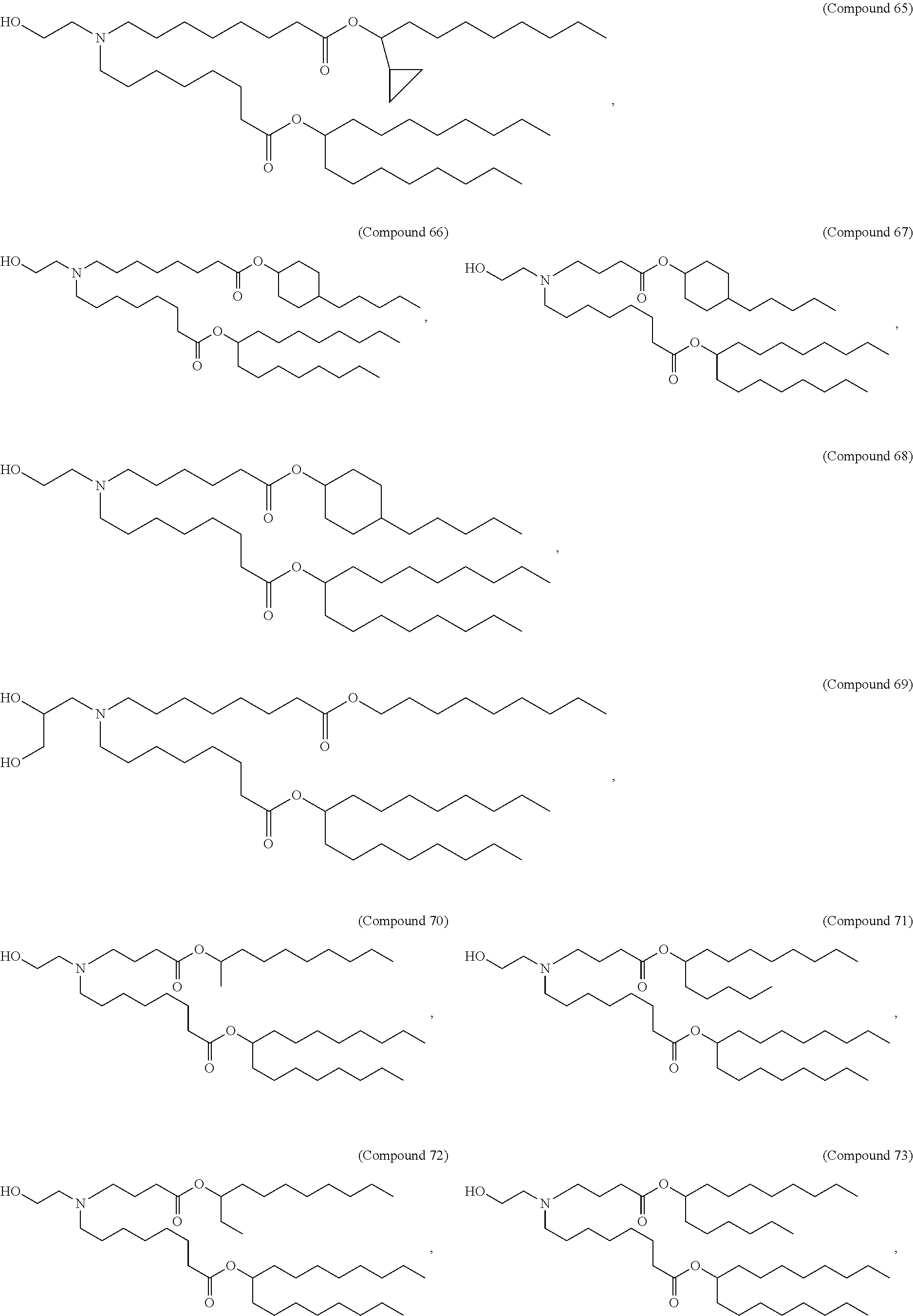

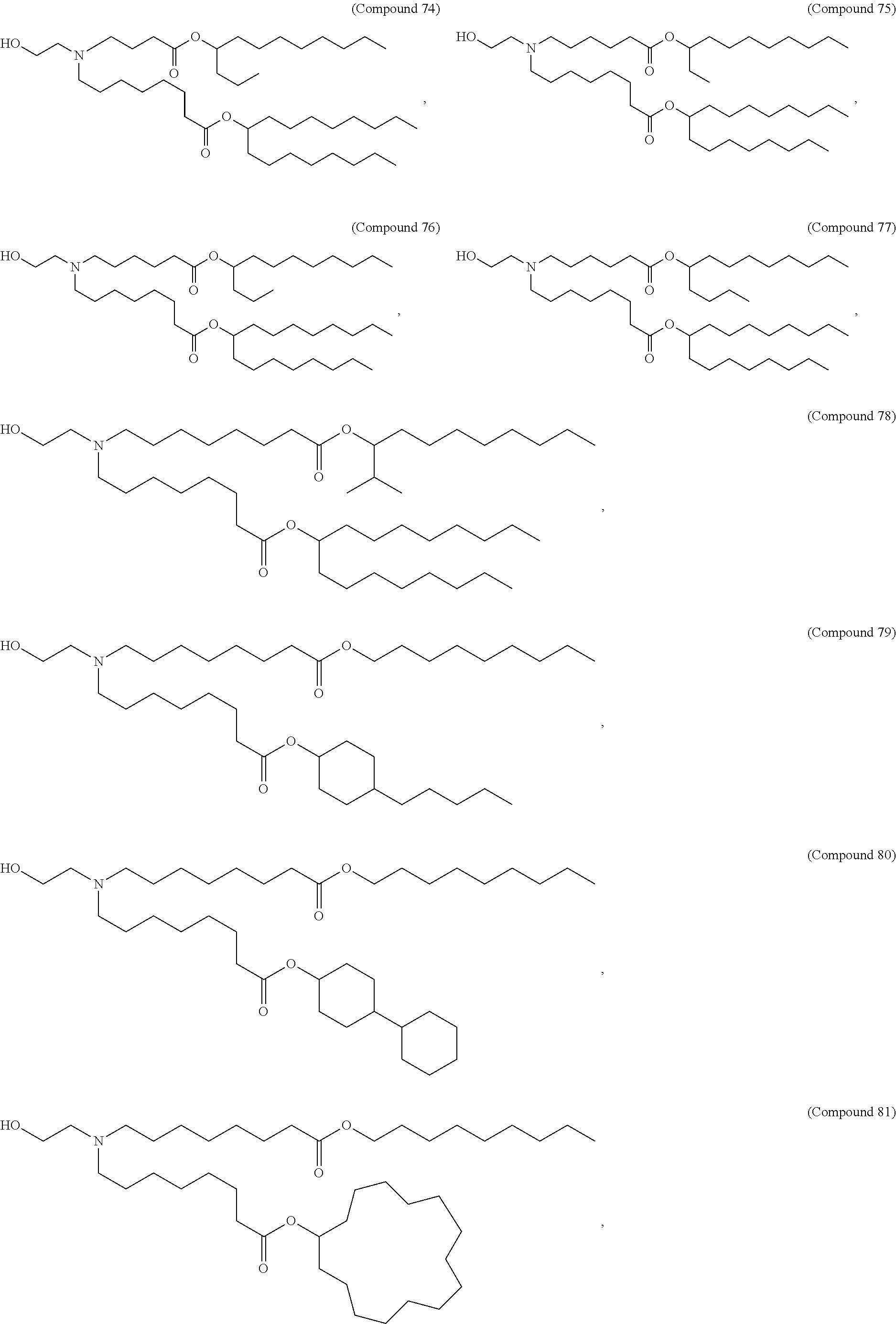

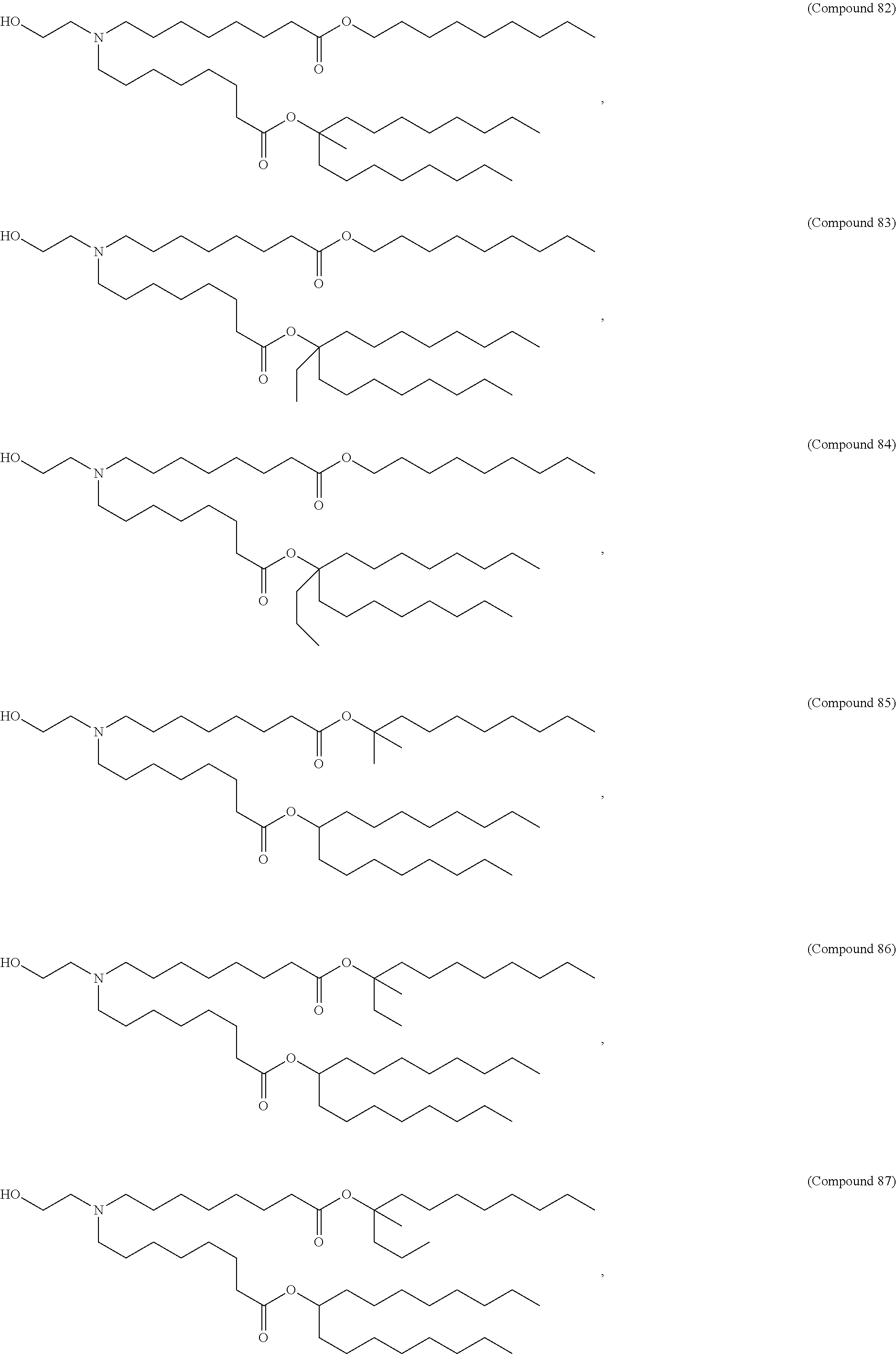 [SMILES: CCCCCCCCCOC(=O)CCCCCCCN(CCO)CCCCCCCC(=O)OC(C)(CCCCCCCC)CCCCCCCC ; CCCCCCCCCOC(=O)CCCCCCCN(CCO)CCCCCCCC(=O)OC(CC)(CCCCCCCC)CCCCCCCC ; CCCCCCCCCOC(=O)CCCCCCCN(CCO)CCCCCCCC(=O)OC(CCC)(CCCCCCCC)CCCCCCCC ; CCCCCCCCC(CCCCCCCC)OC(=O)CCCCCCCN(CCO)CCCCCCCC(=O)OC(C)(C)CCCCCCCC ; CCCCCCCCC(CCCCCCCC)OC(=O)CCCCCCCN(CCO)CCCCCCCC(=O)OC(C)(CC)CCCCCCCC ; CCCCCCCCC(CCCCCCCC)OC(=O)CCCCCCCN(CCO)CCCCCCCC(=O)OC(C)(CCC)CCCCCCCC]

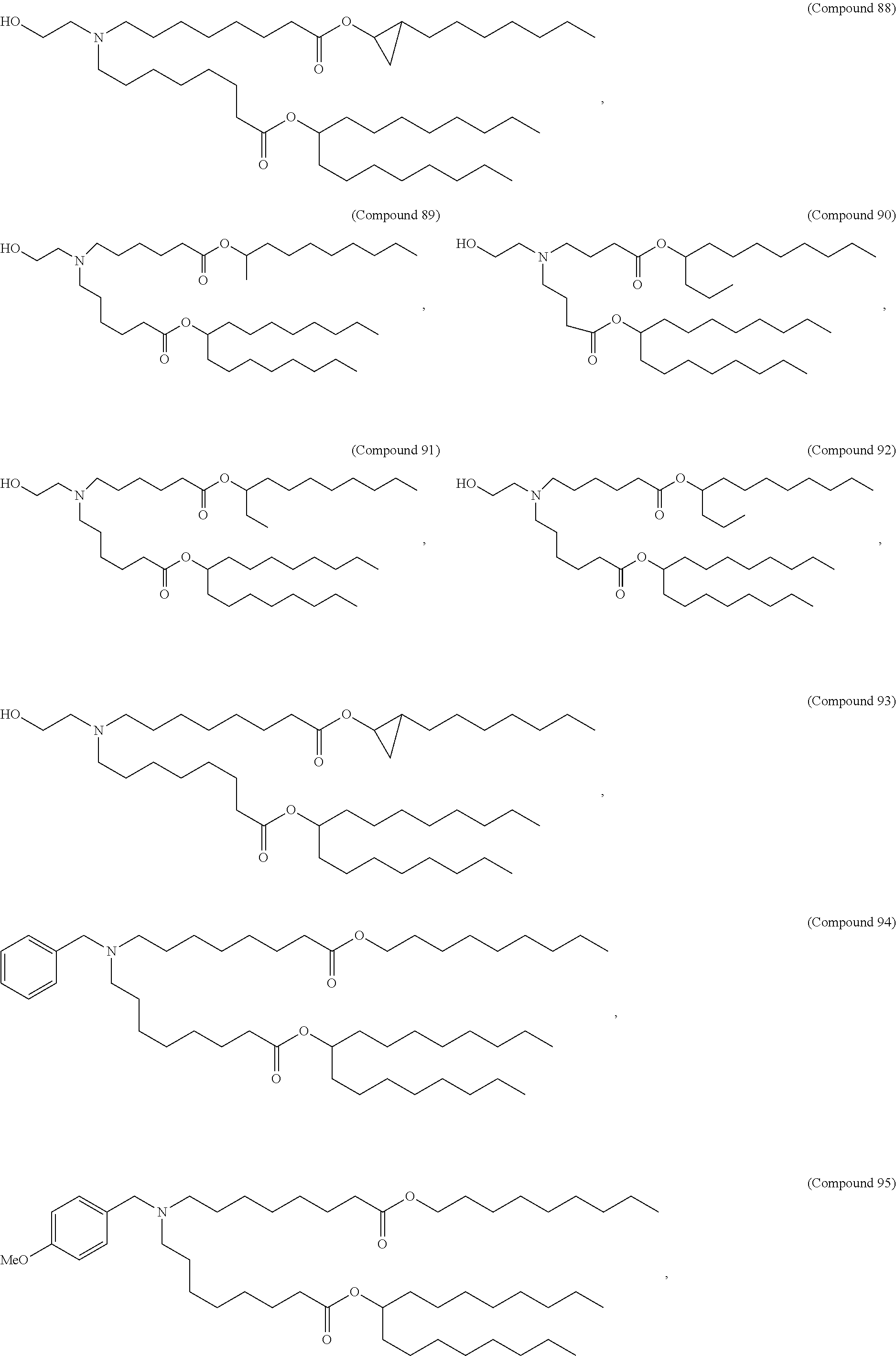

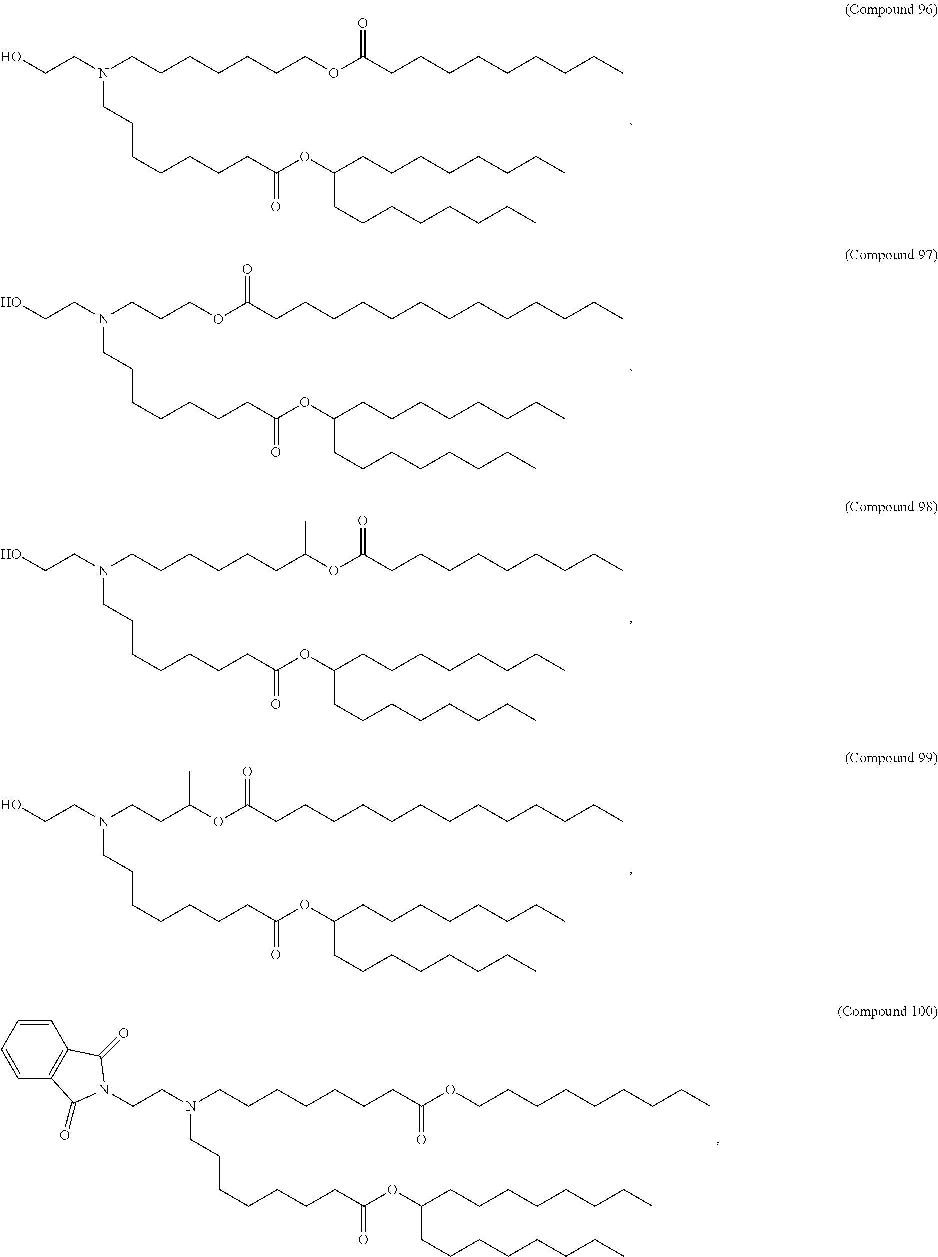 [SMILES: CCCCCCCCCC(=O)OCCCCCCCN(CCO)CCCCCCCC(=O)OC(CCCCCCCC)CCCCCCCC ; CCCCCCCCCCCCCC(=O)OCCCN(CCO)CCCCCCCC(=O)OC(CCCCCCCC)CCCCCCCC ; CCCCCCCCCC(=O)OC(C)CCCCCCN(CCO)CCCCCCCC(=O)OC(CCCCCCCC)CCCCCCCC ; CCCCCCCCCCCCCC(=O)OC(C)CCN(CCO)CCCCCCCC(=O)OC(CCCCCCCC)CCCCCCCC ; CCCCCCCCCOC(=O)CCCCCCCN(CCCCCCCC(=O)OC(CCCCCCCC)CCCCCCCC)CCN1C(=O)c2ccccc2C1=O]

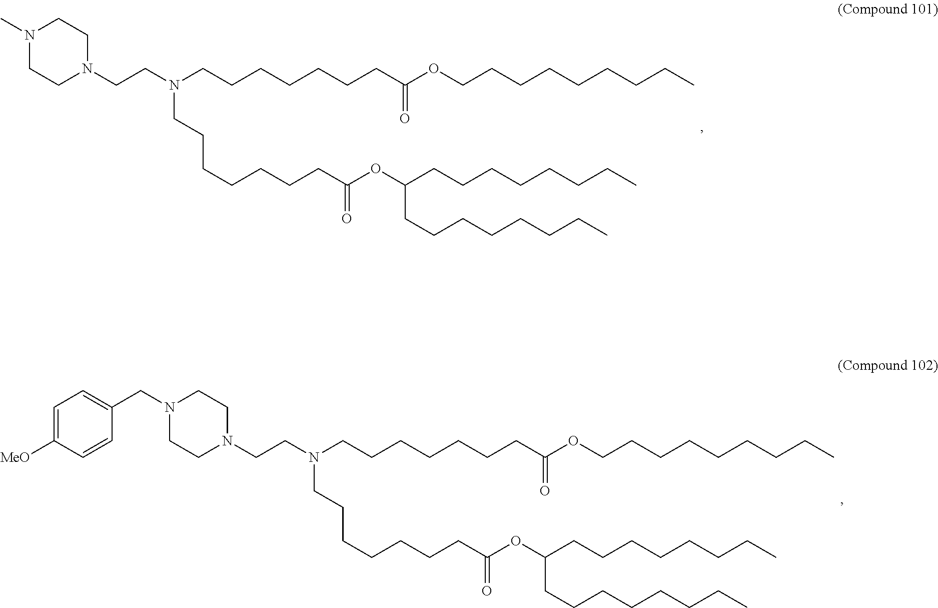 [SMILES: CCCCCCCCCOC(=O)CCCCCCCN(CCCCCCCC(=O)OC(CCCCCCCC)CCCCCCCC)CCN1CCN(C)CC1 ; CCCCCCCCCOC(=O)CCCCCCCN(CCCCCCCC(=O)OC(CCCCCCCC)CCCCCCCC)CCN1CCN(Cc2ccc(OC)cc2)CC1]

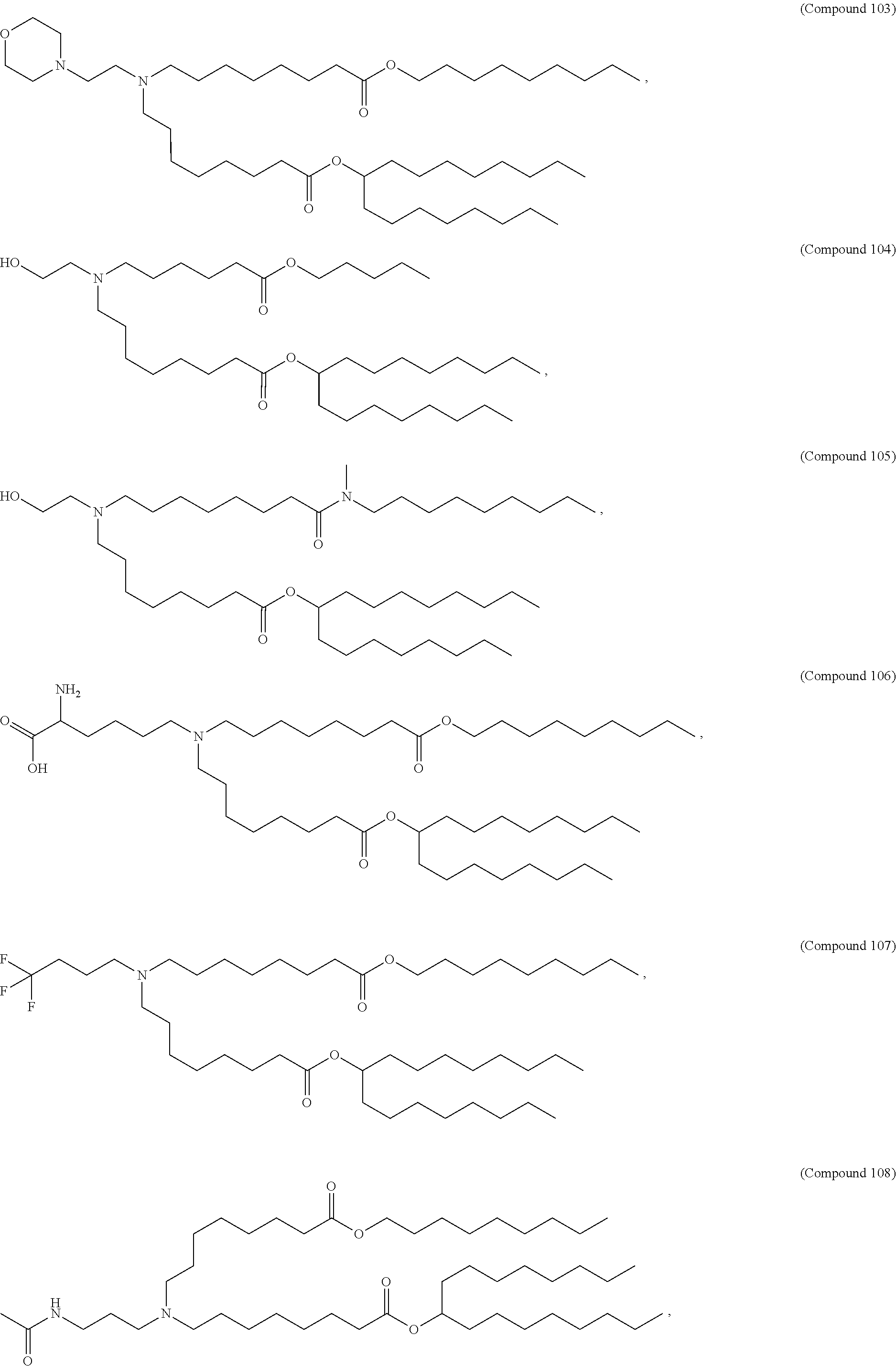

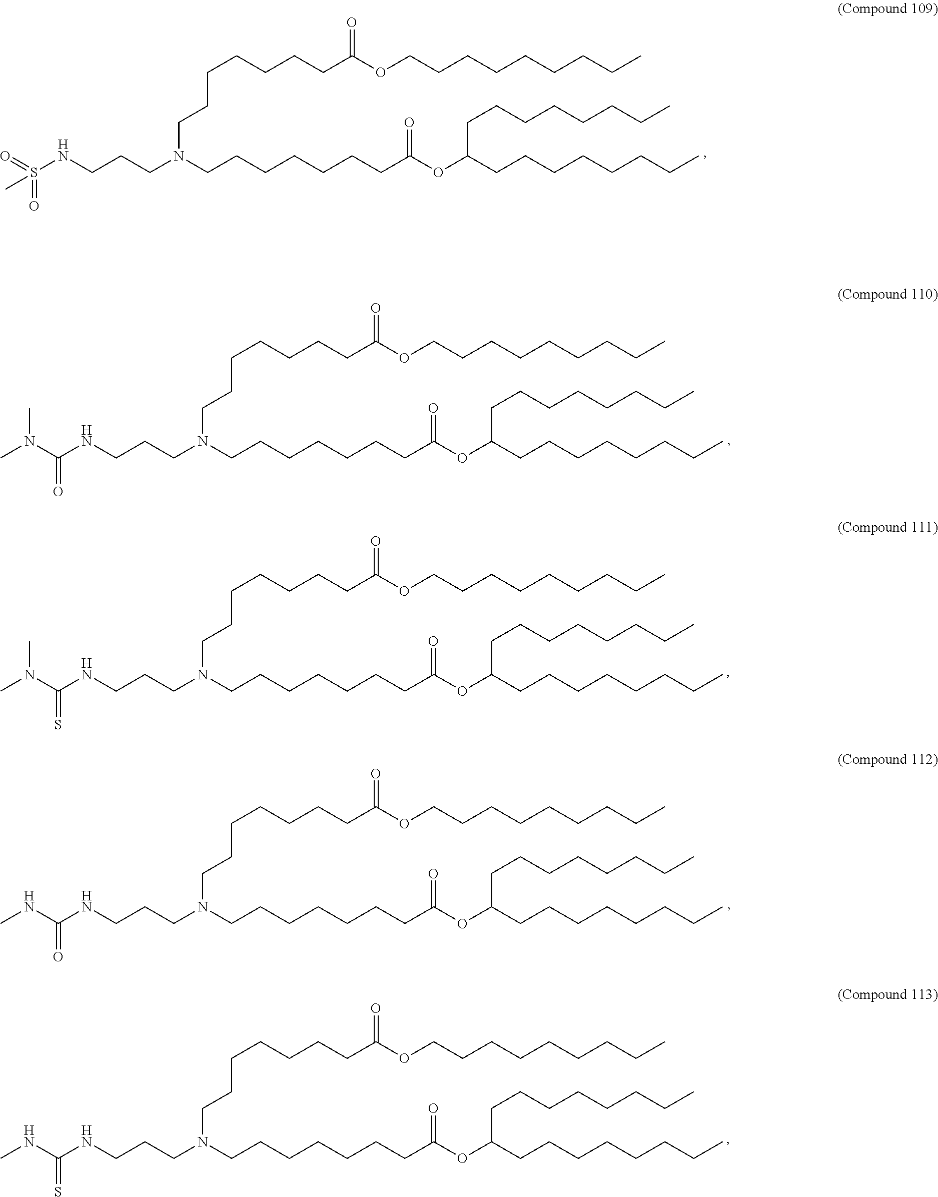

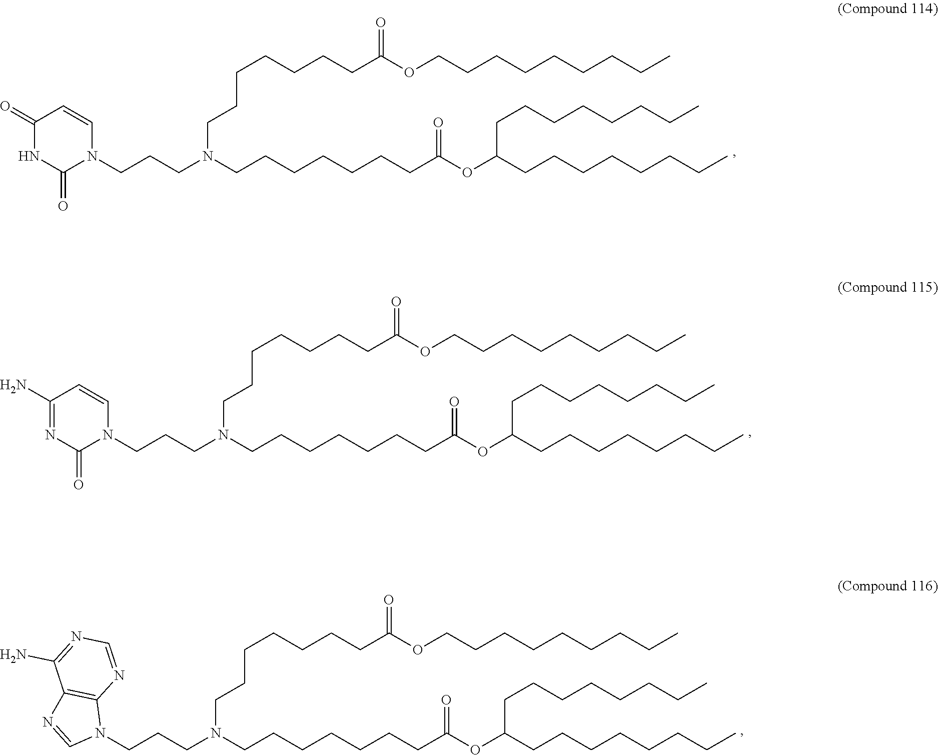 [SMILES: CCCCCCCCCOC(=O)CCCCCCCN(CCCCCCCC(=O)OC(CCCCCCCC)CCCCCCCC)CCCn1ccc(=O)[nH]c1=O ; CCCCCCCCCOC(=O)CCCCCCCN(CCCCCCCC(=O)OC(CCCCCCCC)CCCCCCCC)CCCn1ccc(N)nc1=O ; CCCCCCCCCOC(=O)CCCCCCCN(CCCCCCCC(=O)OC(CCCCCCCC)CCCCCCCC)CCCn1cnc2c(N)ncnc21]

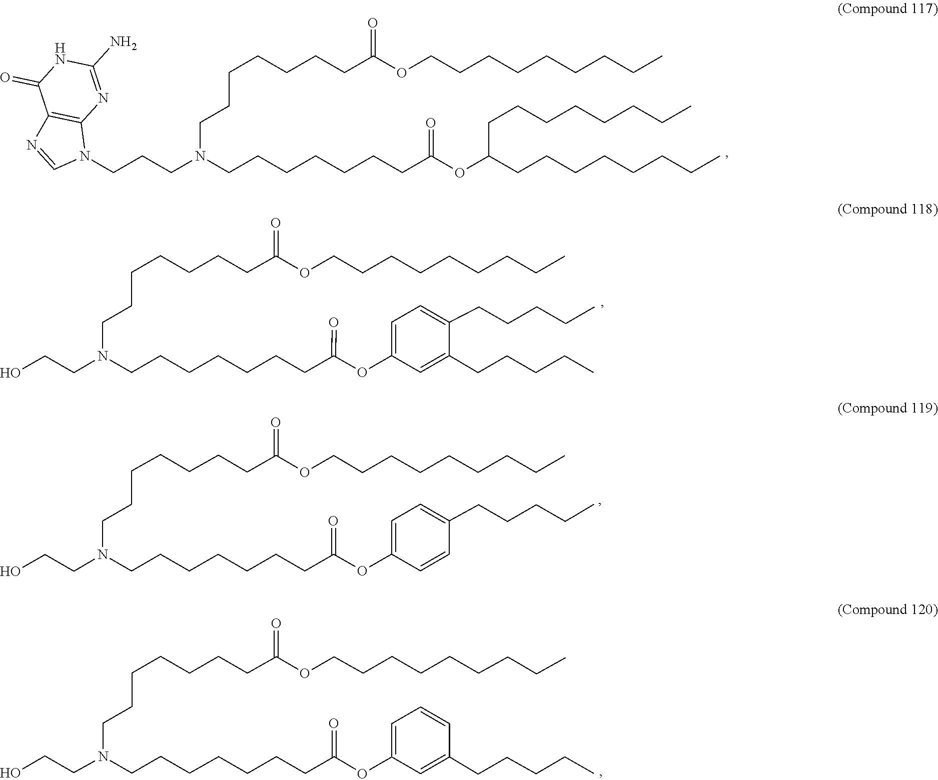 [SMILES: CCCCCCCCCOC(=O)CCCCCCCN(CCCCCCCC(=O)OC(CCCCCCCC)CCCCCCCC)CCCn1cnc2c(=O)[nH]c(N)nc21 ; CCCCCCCCCOC(=O)CCCCCCCN(CCO)CCCCCCCC(=O)Oc1ccc(CCCCC)c(CCCCC)c1 ; CCCCCCCCCOC(=O)CCCCCCCN(CCO)CCCCCCCC(=O)Oc1ccc(CCCCC)cc1 ; CCCCCCCCCOC(=O)CCCCCCCN(CCO)CCCCCCCC(=O)Oc1cccc(CCCCC)c1]

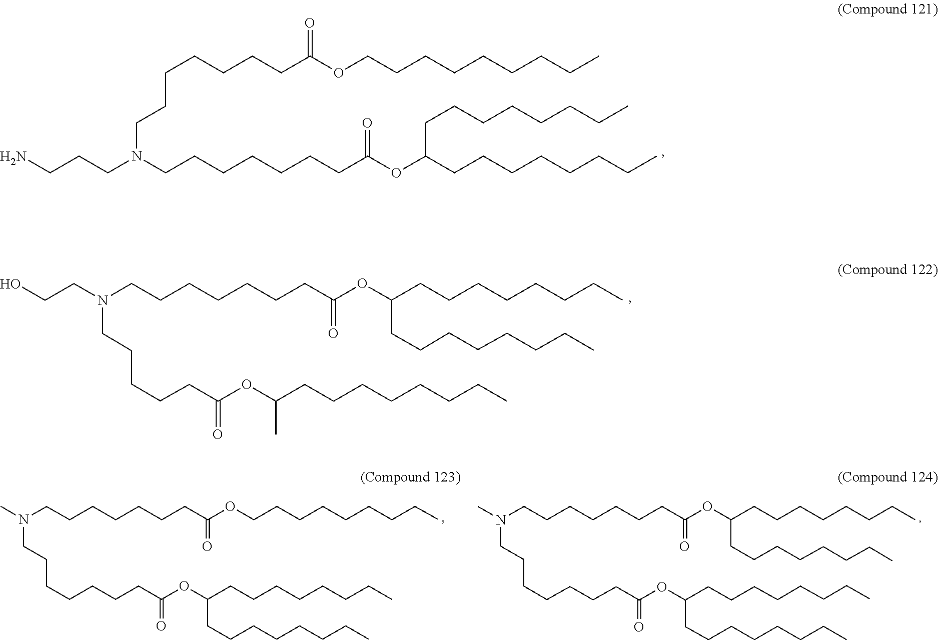

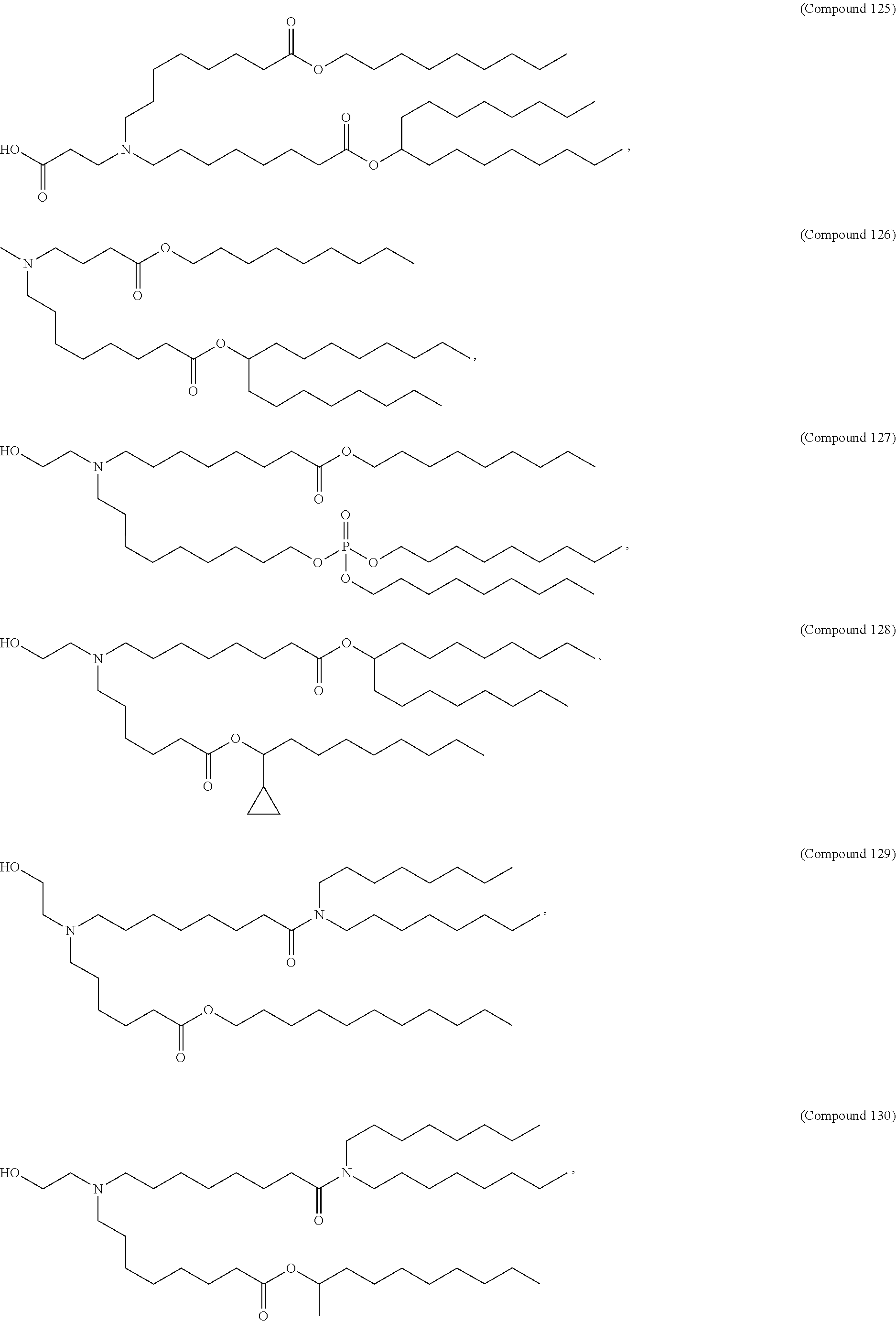

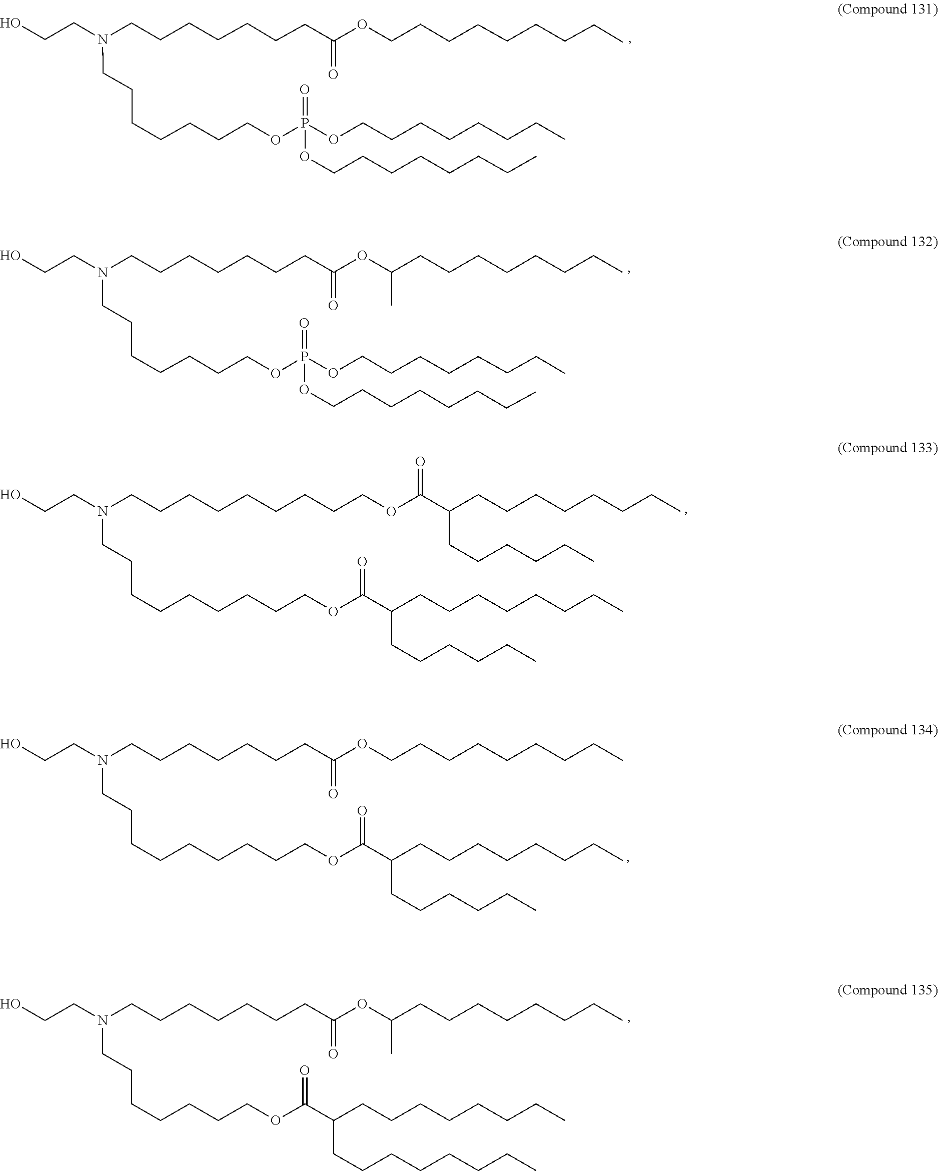 [SMILES: CCCCCCCCCOC(=O)CCCCCCCN(CCO)CCCCCCCOP(=O)(OCCCCCCCC)OCCCCCCCC ; CCCCCCCCOP(=O)(OCCCCCCCC)OCCCCCCCN(CCO)CCCCCCCC(=O)OC(C)CCCCCCCC ; CCCCCCCCC(CCCCCC)C(=O)OCCCCCCCCCN(CCO)CCCCCCCCCOC(=O)C(CCCCCC)CCCCCCCC ; CCCCCCCCCOC(=O)CCCCCCCN(CCO)CCCCCCCCCOC(=O)C(CCCCCC)CCCCCCCC ; CCCCCCCCC(C)OC(=O)CCCCCCCN(CCO)CCCCCCCOC(=O)C(CCCCCCCC)CCCCCCCC]

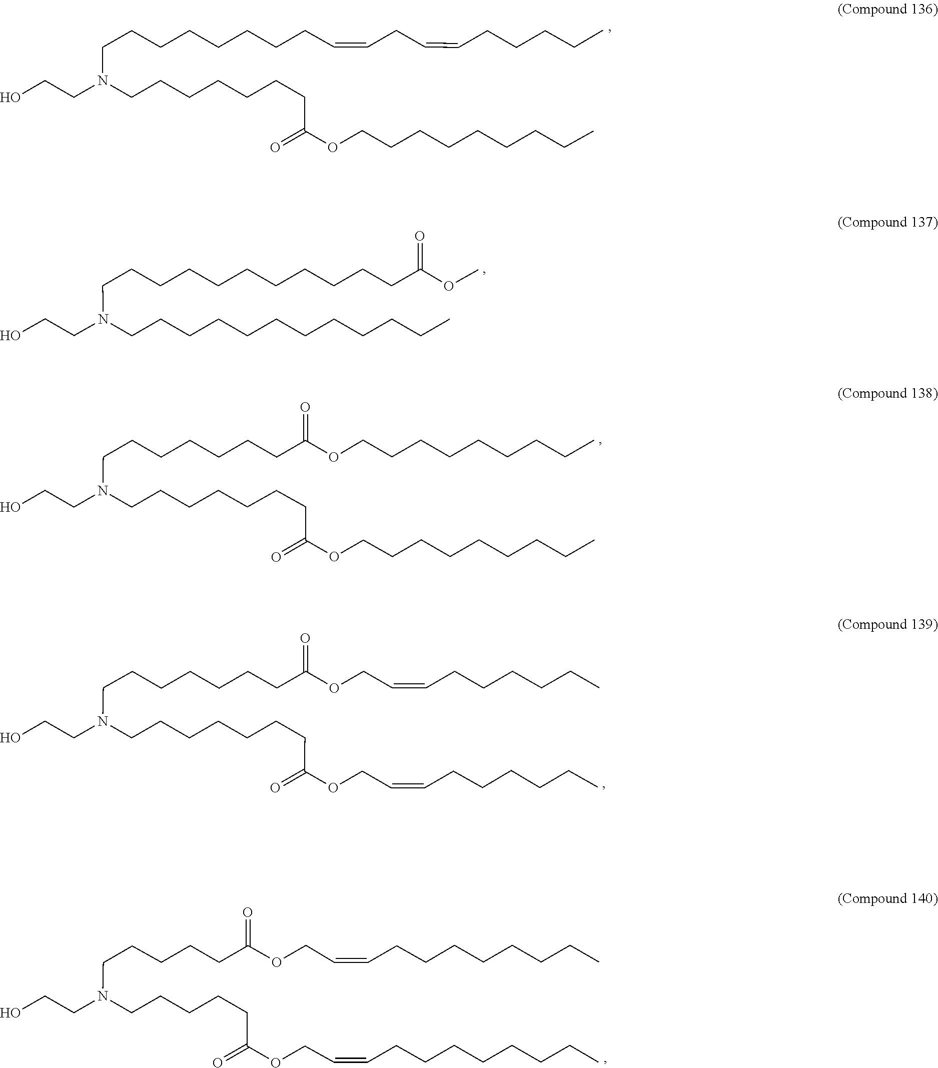 [SMILES: CCCCC/C=C\C/C=C\CCCCCCCCN(CCO)CCCCCCCC(=O)OCCCCCCCCC ; CCCCCCCCCCCCN(CCO)CCCCCCCCCCCC(=O)OC ; CCCCCCCCCOC(=O)CCCCCCCN(CCO)CCCCCCCC(=O)OCCCCCCCCC ; CCCCCC/C=C\COC(=O)CCCCCCCN(CCO)CCCCCCCC(=O)OC/C=C\CCCCCC ; CCCCCCCC/C=C\COC(=O)CCCCCN(CCO)CCCCCC(=O)OC/C=C\CCCCCCCC]

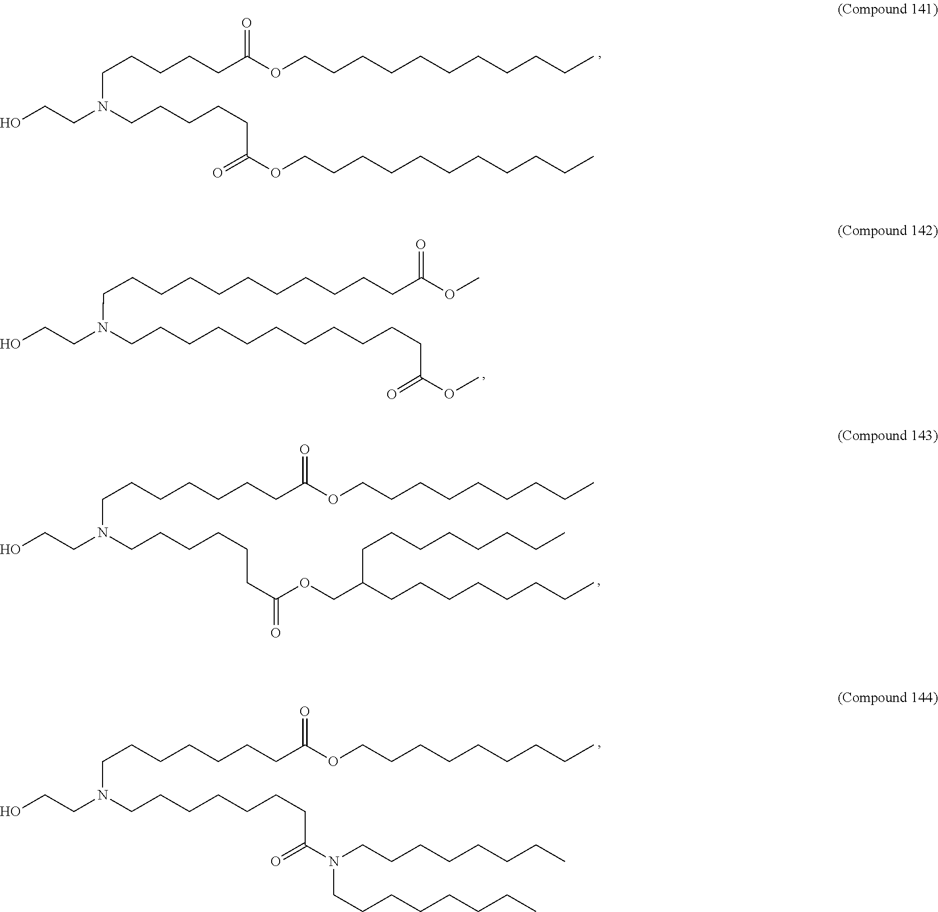 [SMILES: CCCCCCCCCCCOC(=O)CCCCCN(CCO)CCCCCC(=O)OCCCCCCCCCCC ; COC(=O)CCCCCCCCCCCN(CCO)CCCCCCCCCCCC(=O)OC ; CCCCCCCCCOC(=O)CCCCCCCN(CCO)CCCCCCC(=O)OCC(CCCCCCCC)CCCCCCCC ; CCCCCCCCCOC(=O)CCCCCCCN(CCO)CCCCCCCC(=O)N(CCCCCCCC)CCCCCCCC]

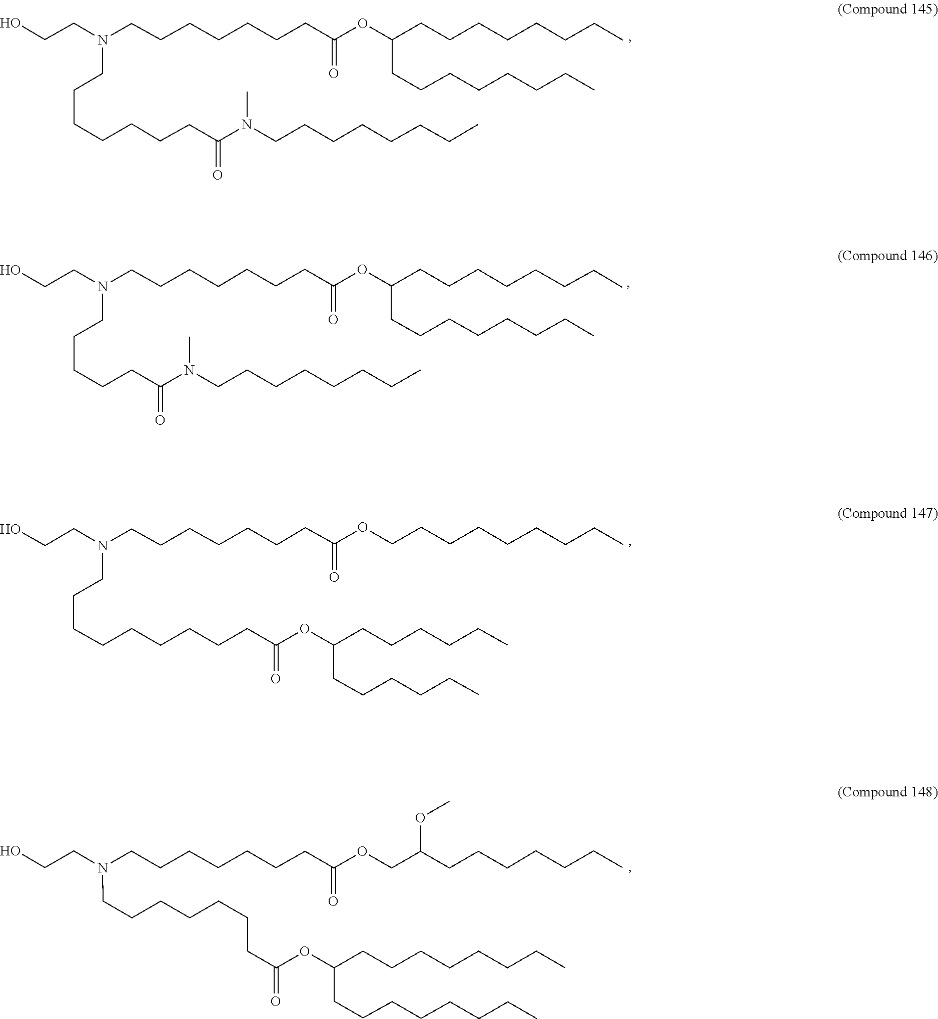 [SMILES: CCCCCCCCC(CCCCCCCC)OC(=O)CCCCCCCN(CCO)CCCCCCCC(=O)N(C)CCCCCCCC ; CCCCCCCCC(CCCCCCCC)OC(=O)CCCCCCCN(CCO)CCCCCC(=O)N(C)CCCCCCCC ; CCCCCCCCCOC(=O)CCCCCCCN(CCO)CCCCCCCCCC(=O)OC(CCCCCC)CCCCCC ; CCCCCCCCC(CCCCCCCC)OC(=O)CCCCCCCN(CCO)CCCCCCCC(=O)OCC(CCCCCCC)OC]

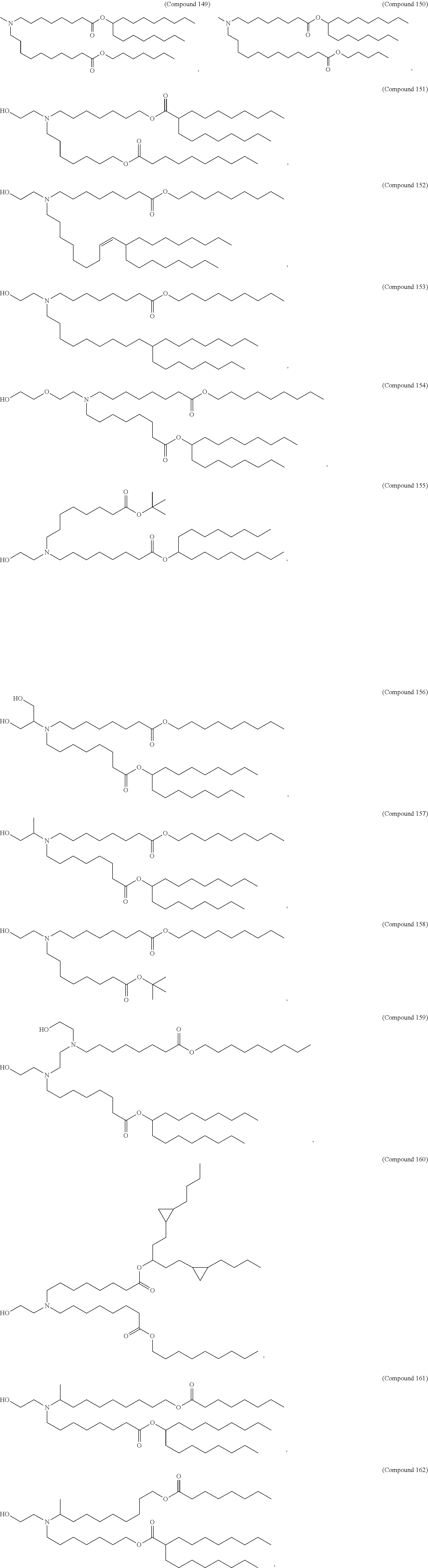

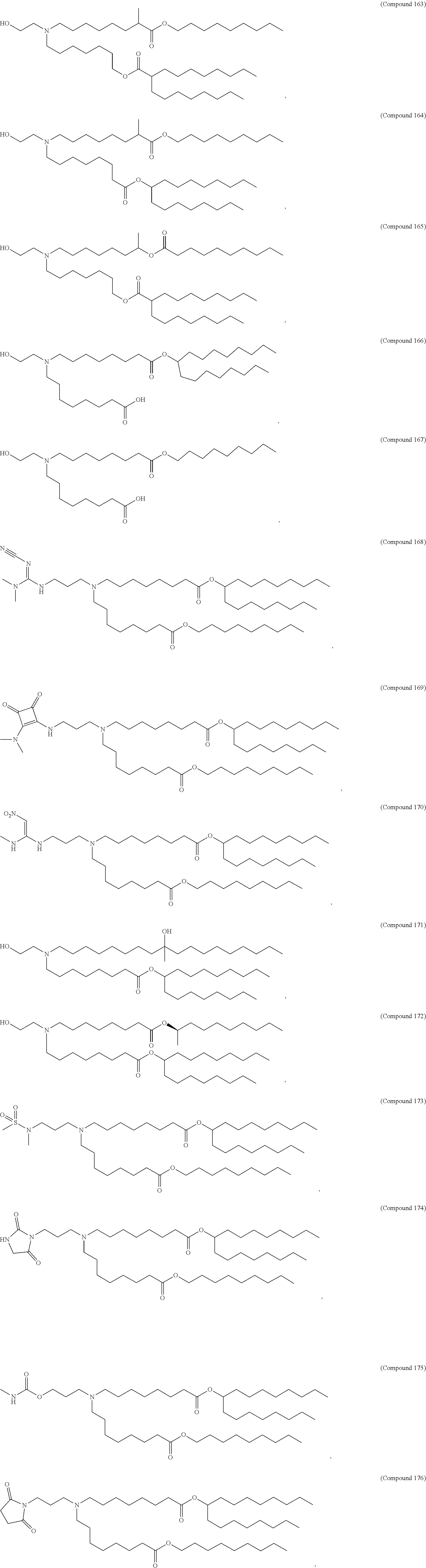

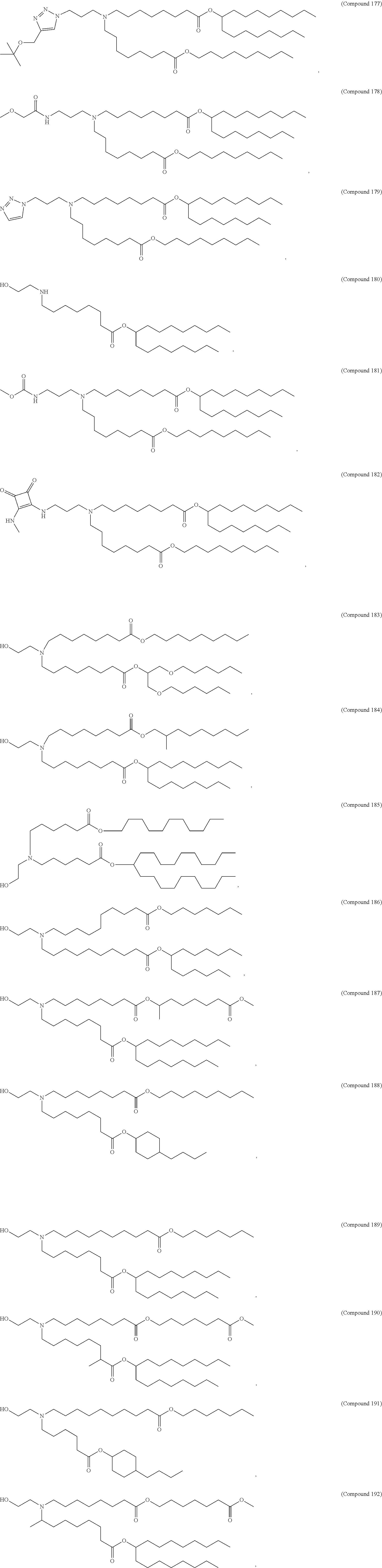

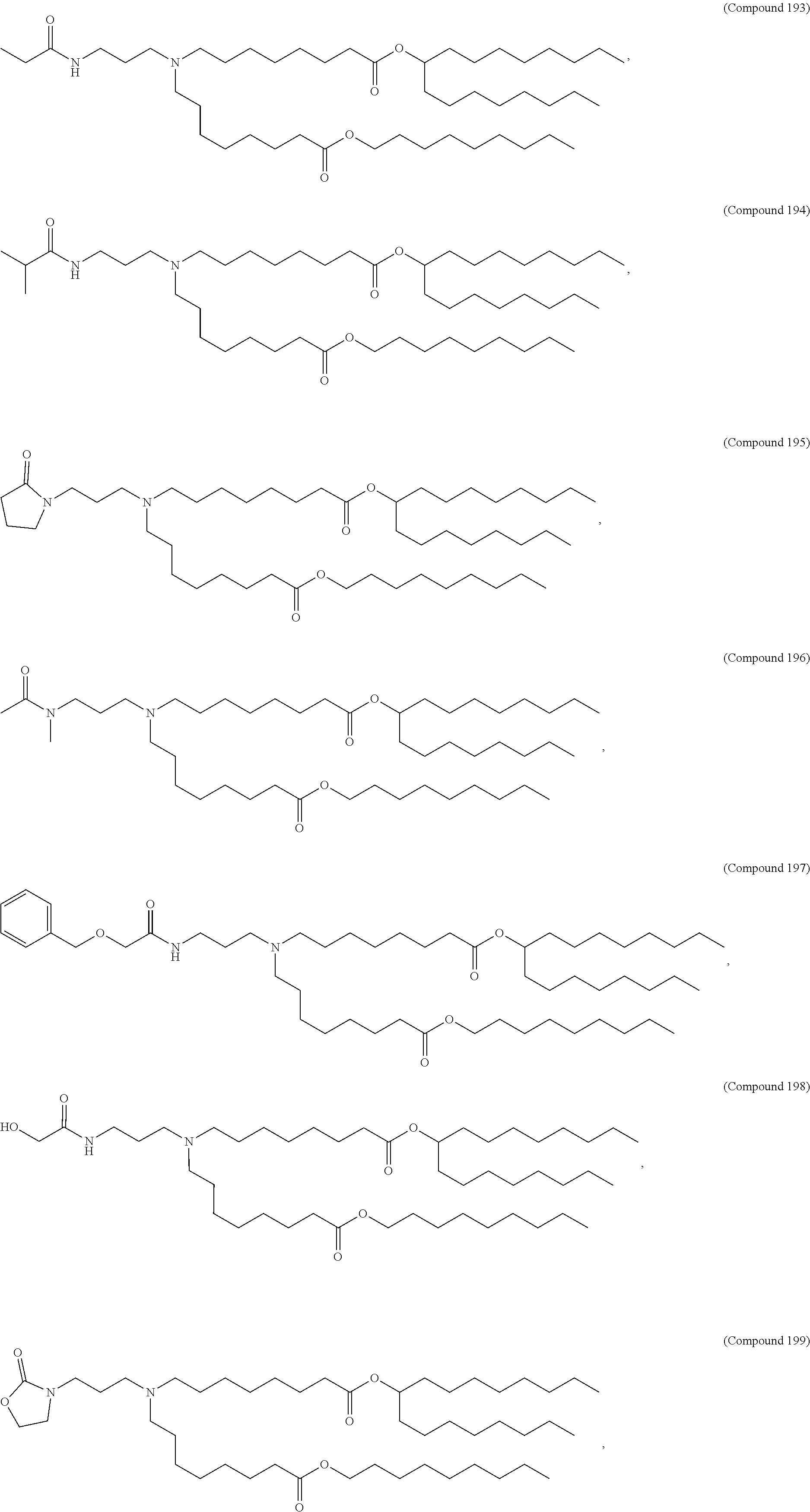

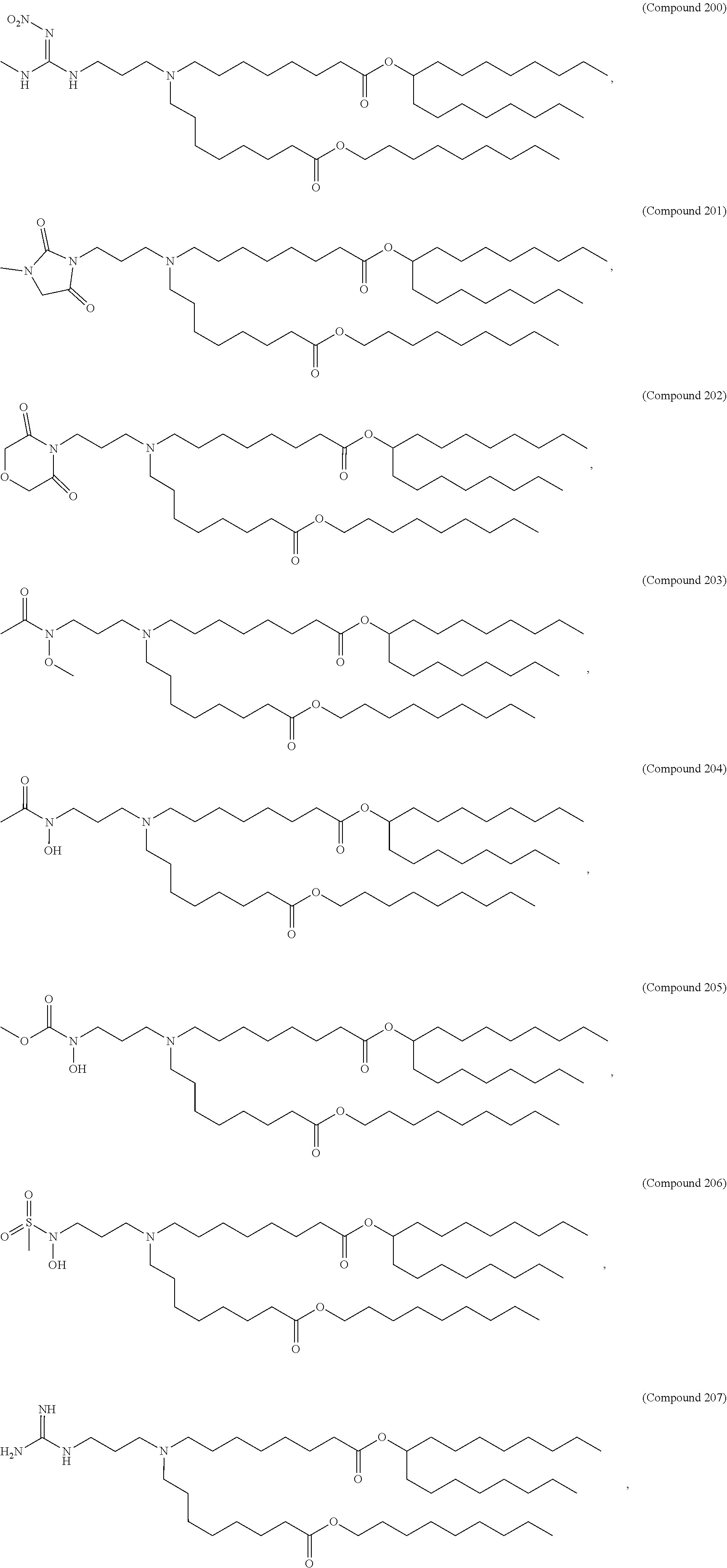

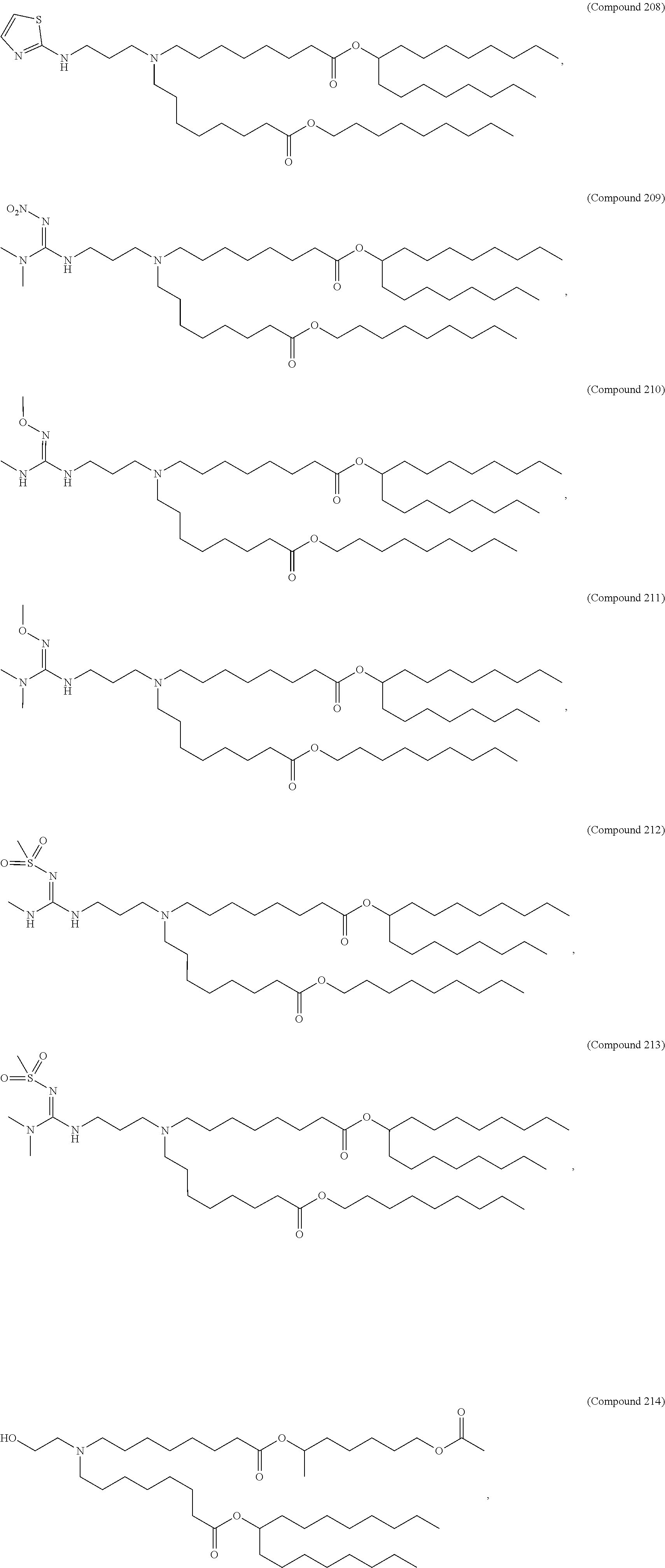

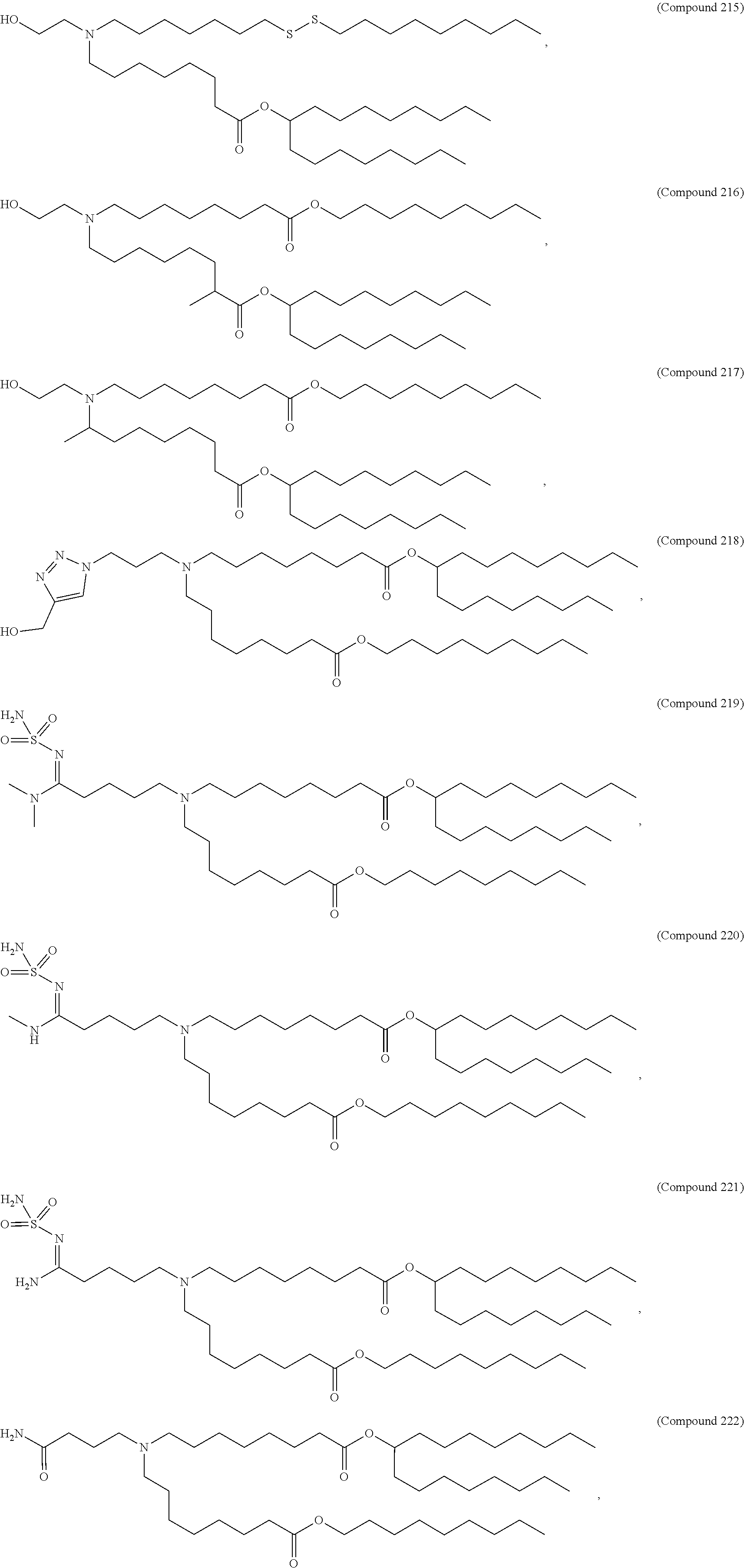

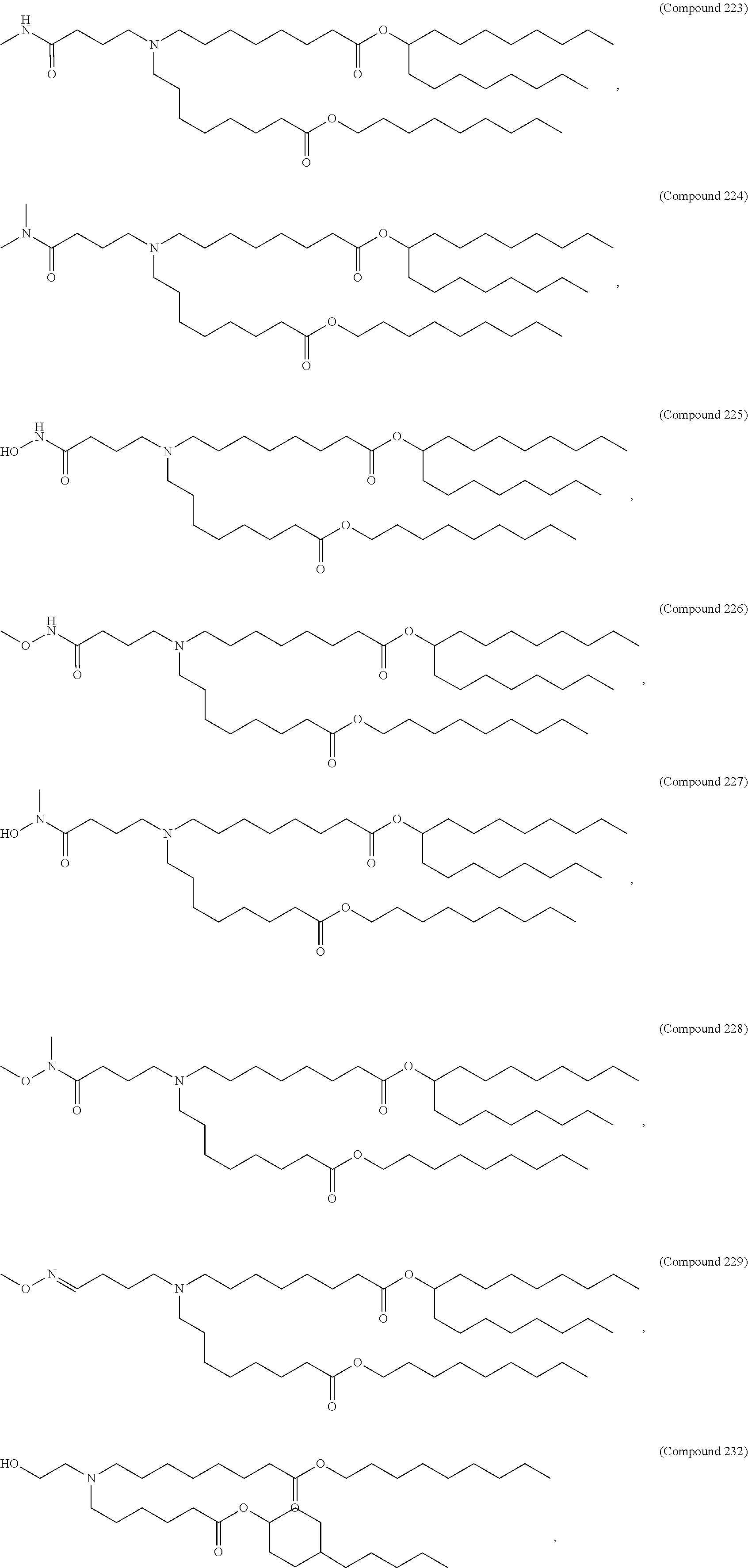

and salts and isomers thereof.

In some embodiments, the ionizable lipid is compound 429:

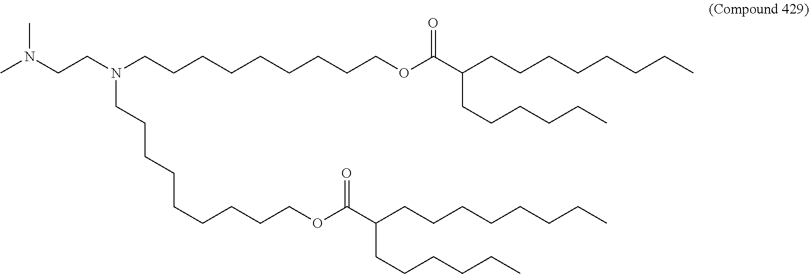 [SMILES: CCCCCCCCC(CCCCCC)C(=O)OCCCCCCCCCN(CCCCCCCCCOC(=O)C(CCCCCC)CCCCCCCC)CCN(C)C]

or a salt thereof.

In some embodiments, the ionizable lipid is compound 18:

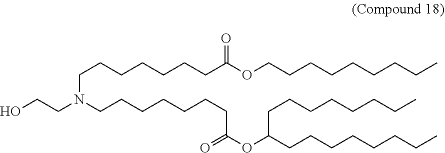

or a salt thereof.

In some embodiments, the ionizable lipid is a compound of Formula (X):

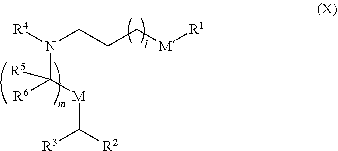

or an N-oxide or a salt thereof, wherein:
R¹ is

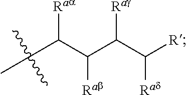

wherein

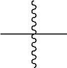

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is selected from —(CH₂)ₙOH and

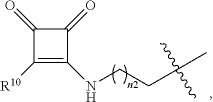

wherein n is selected from 1, 2, 3, 4, and 5;
wherein

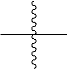

denotes a point of attachment,
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R¹ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (X), or an N-oxide or a salt thereof, wherein:
R¹ is

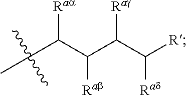

wherein

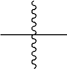

denotes a point of attachment;
R^{aα}, R^{aβ}, R^{aγ}, and R^{aδ} are each H;
R² and R³ are each C₁₋₁₄ alkyl;
R⁴ is —(CH₂)nOH;
n is 2;
each R⁵ is H;
each R⁶ is H;
M and M′ are each —C(O)O—;
R¹ is C₁₋₁₂ alkyl;
l is 5; and
m is 7.

In some embodiments, the ionizable lipid is a compound of Formula (X), or an N-oxide or a salt thereof, wherein:
R¹ is

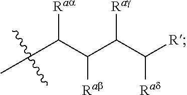

wherein

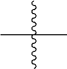

denotes a point of attachment;
R^{aα}, R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R¹ is C₁₋₁₂ alkyl;
l is 3; and
m is 7.

In some embodiments, the ionizable lipid is a compound of Formula (X), or an N-oxide or a salt thereof, wherein:
R¹ is

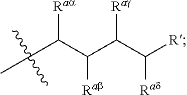

wherein

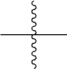

denotes a point of attachment;
R^{aα} is C₂₋₁₂ alkyl;
R^{aβ}, R^{aγ}, and R^{aδ} are each H;
R² and R³ are each C₁₋₁₄ alkyl;
R⁴ is

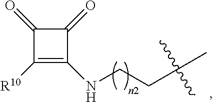

R¹⁰ is —NH(C₁₋₆ alkyl);
n2 is 2;
each R⁵ is H;
each R⁶ is H;
M and M′ are each —C(O)O—;
R¹ is C₁₋₁₂ alkyl;
l is 5; and
m is 7.

In some embodiments, the ionizable lipid is a compound of Formula (X), or an N-oxide or a salt thereof, wherein:
R¹ is

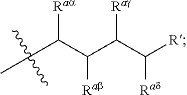

wherein

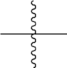

denotes a point of attachment;
R^{aα}, R^{aβ}, and R^{aδ} are each H;
R^{aγ} is C₂₋₁₂ alkyl;
R² and R³ are each C₁₋₁₄ alkyl;
R⁴ is —(CH₂)ₙOH;
n is 2;
each R⁵ is H;
each R⁶ is H;
M and M′ are each —C(O)O—;
R¹ is C₁₋₁₂ alkyl;
l is 5; and
m is 7.

In some embodiments, the ionizable lipid is selected from:

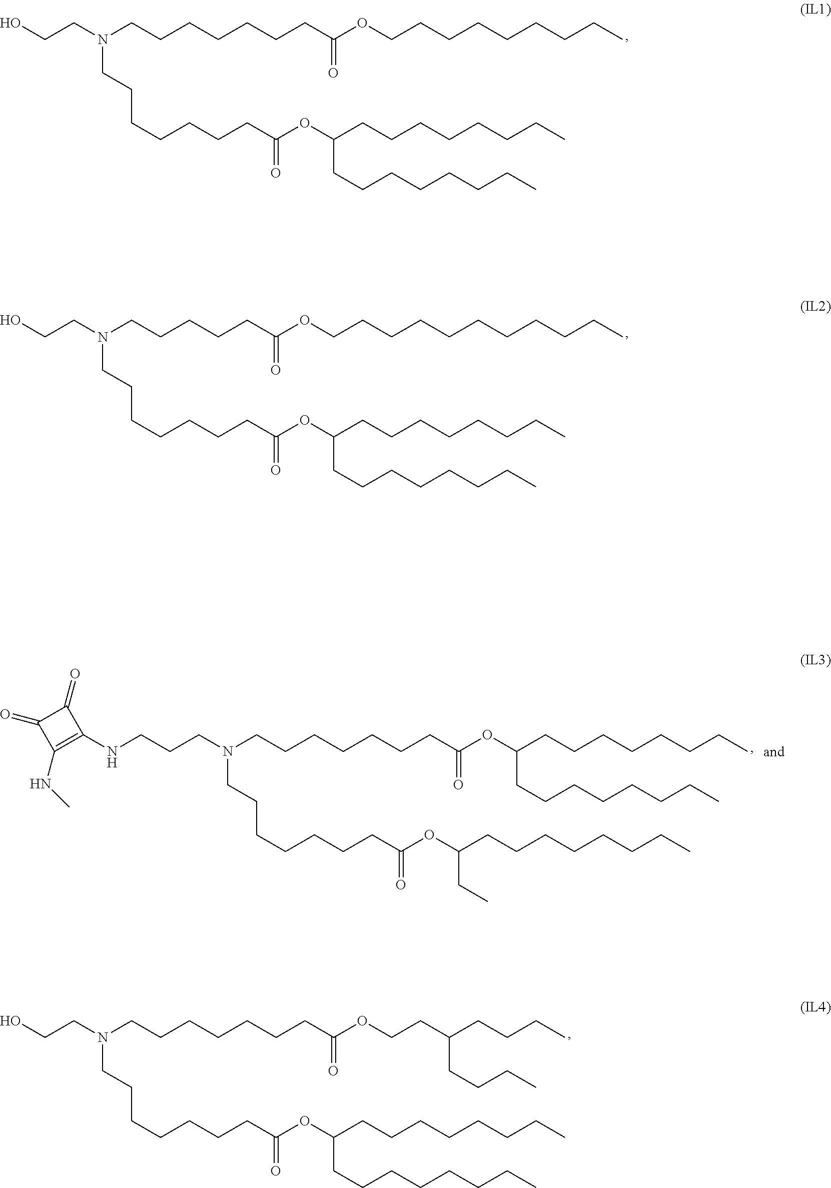

or an N-oxide or a salt thereof.

In some embodiments, the ionizable lipid is the compound:

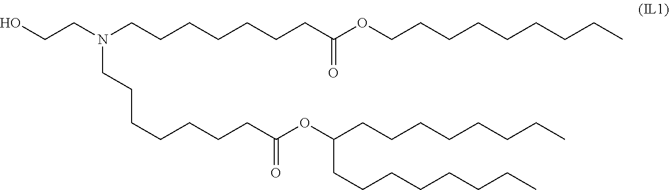 [SMILES: CCCCCCCCCOC(=O)CCCCCCCN(CCO)CCCCCCCC(=O)OC(CCCCCCCC)CCCCCCCC]

or an N-oxide or a salt thereof.

In some embodiments, the ionizable lipid is the compound:

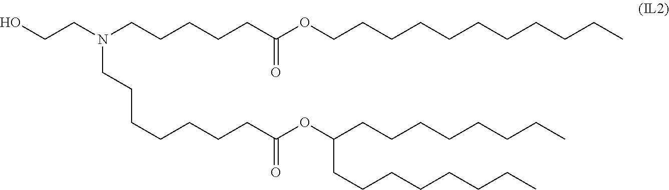 [SMILES: CCCCCCCCCCCOC(=O)CCCCCN(CCO)CCCCCCCC(=O)OC(CCCCCCCC)CCCCCCCC]

or an N-oxide or a salt thereof.

In some embodiments, the ionizable lipid is the compound:

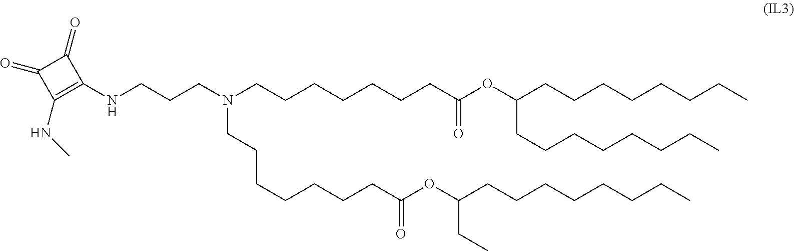 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or an N-oxide or a salt thereof.

In some embodiments, the ionizable lipid is the compound:

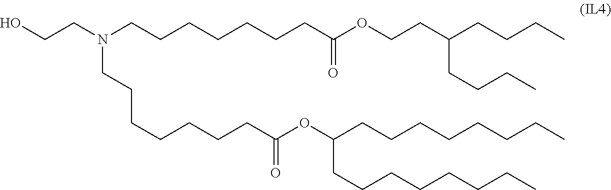 [SMILES: CCCCCCCCC(CCCCCCCC)OC(=O)CCCCCCCN(CCO)CCCCCCCC(=O)OCCC(CCCC)CCCC]

or an N-oxide or a salt thereof.

In some embodiments, the ionizable lipid is a compound of Formula (X):

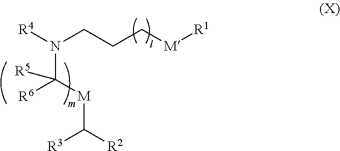

or an N-oxide or a salt thereof, wherein:
R¹ is:

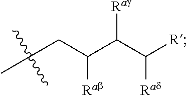

wherein

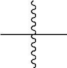

denotes a point of attachment;
R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is selected from —(CH₂)ₙOH and

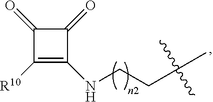

wherein

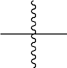

denotes a point of attachment;
wherein n is selected from 1, 2, 3, 4, and 5;
wherein R¹⁰ is —N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R¹ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (X):

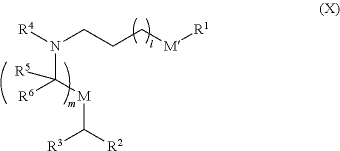

or an N-oxide or a salt thereof, wherein:
R¹ is:

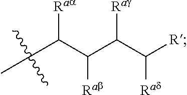

wherein

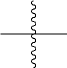

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is —(CH₂)ₙOH, wherein n is selected from 1, 2, 3, 4, and 5;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R¹ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (X), or an N-oxide or a salt thereof, wherein:
R¹ is

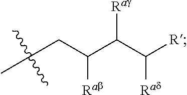

wherein

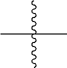

denotes a point of attachment;
R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R¹ is C₁₋₁₂ alkyl;
l is 5; and
m is 7.

In some embodiments, the ionizable lipid is a compound of Formula (X), or an N-oxide or a salt thereof, wherein:
R¹ is

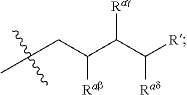

wherein

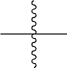

denotes a point of attachment;
R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R¹ is C₁₋₁₂ alkyl;
l is 3; and
m is 7.

In some embodiments, the ionizable lipid is a compound of Formula (X), or an N-oxide or a salt thereof, wherein:

R¹ is

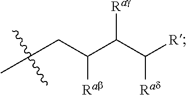

wherein

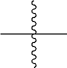

denotes a point of attachment;
R^{aβ} and R^{aδ} are each H;
R^{aγ} is C₂₋₁₂ alkyl;
R² and R³ are each C₁₋₁₄ alkyl;
R⁴ is —(CH₂)ₙOH;
n is 2;
each R⁵ is H;
each R⁶ is H;
M and M′ are each —C(O)O—;
R¹ is C₁₋₁₂ alkyl;
l is 5; and
m is 7.

In some embodiments, the ionizable lipid is a compound of Formula (X):

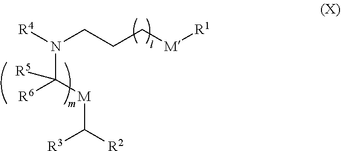

or an N-oxide or a salt thereof, wherein:
R¹ is:

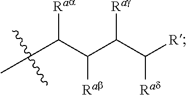

wherein

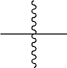

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is

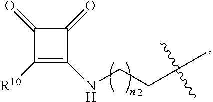

wherein

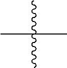

denotes a point of attachment;
wherein R¹⁰ is —N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R¹ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments:
R¹ is

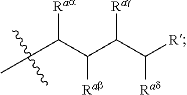

wherein

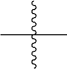

denotes a point of attachment;
R^{aβ}, R^{aγ}, and R^{aδ} are each H;
R^{aα} is C₂₋₁₂ alkyl;
R² and R³ are each C₁₋₁₄ alkyl;
R⁴ is

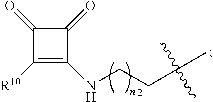

wherein

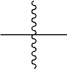

denotes a point of attachment;
wherein R¹⁰ is —NH(C₁₋₆ alkyl);
wherein n2 is 2;
each R⁵ is H;
each R⁶ is H;
M and M′ are each —C(O)O—;
R¹ is C₁₋₁₂ alkyl;
l is 5; and
m is 7.

In some embodiments, the ionizable lipid of Formula (X) is:

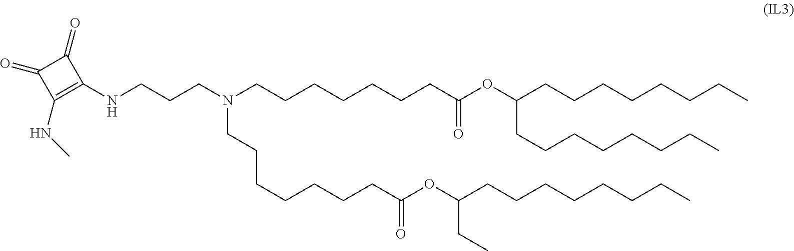 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or an N-oxide or a salt thereof.

In some embodiments, the ionizable lipid is a compound of Formula (XI):

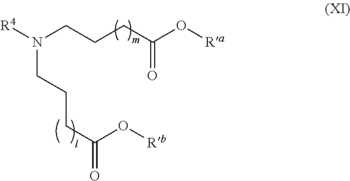

or an N-oxide or a salt thereof, wherein:
R′^{a} is R′^{branched} or R′^{cyclic}; wherein
R′^{branched} is

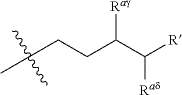

### and R′^{cyclic} is:

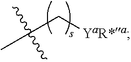

and
R′^{b} is:

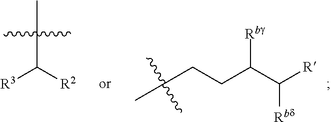

wherein

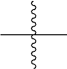

denotes a point of attachment;
R^{aγ} and R^{aδ} are each independently selected from H, C₁₋₁₂ alkyl, and C₂₋₁₂ alkenyl, wherein at least one of R^{aγ} and R^{aδ} is selected from C₁₋₁₂ alkyl and C₂₋₁₂ alkenyl;
R^{bγ} and R^{bδ} are each independently selected from H, C₁₋₁₂ alkyl, and C₂₋₁₂ alkenyl, wherein at least one of R^{bγ} and R^{bδ} is selected from C₁₋₁₂ alkyl and C₂₋₁₂ alkenyl;
R² and R³ are each independently selected from the C₁₋₁₄ alkyl and C₂₋₁₄ alkenyl;
R⁴ is selected from —(CH₂)ₙOH and

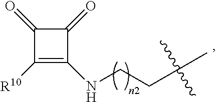

wherein

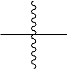

denotes a point of attachment;
wherein n is selected from 1, 2, 3, 4, and 5;
wherein R¹⁰ is —N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R′ independently is C₁₋₁₂ alkyl or C₂₋₁₂ alkenyl;
Y^{a} is a C₃₋₆ carbocycle;
R*″^{a} is selected from C₁₋₁₅ alkyl and C₂₋₁₅ alkenyl;
s is 2 or 3;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (XI):

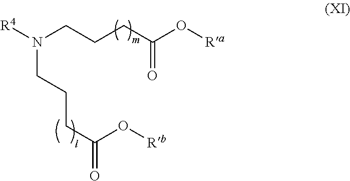

or an N-oxide or a salt thereof, wherein:
R′^{a} is R′^{branched} or R′^{cyclic}; wherein
R′^{branched} is

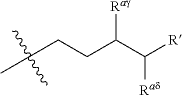

### and R′^{b} is:

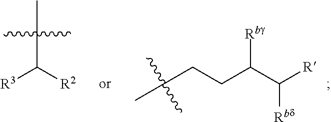

wherein

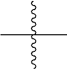

denotes a point of attachment;
R^{aγ} and R^{aδ} are each independently selected from H, C₁₋₁₂ alkyl, and C₂₋₁₂ alkenyl, wherein at least one of R^{aγ} and R^{aδ} is selected from C₁₋₁₂ alkyl and C₂₋₁₂ alkenyl;
R^{bγ} and R^{bδ} are each independently selected from H, C₁₋₁₂ alkyl, and C₂₋₁₂ alkenyl, wherein at least one of R^{bγ} and R^{bδ} is selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

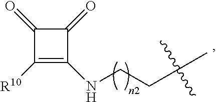

wherein

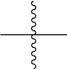

denotes a point of attachment;
wherein n is selected from of 1, 2, 3, 4, and 5;
wherein R¹⁰ is —N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R¹ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (XI):

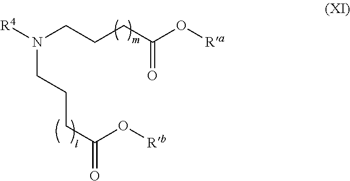

or an N-oxide or a salt thereof, wherein:
R′^{a} is R′^{branched} or R′^{cyclic}; wherein
R′^{branched} is:

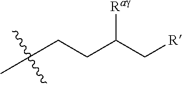

### and R′^{b} is:

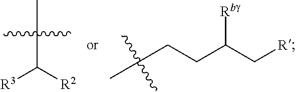

wherein

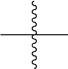

denotes a point of attachment;
R^{aγ} and R^{bγ} are each independently selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

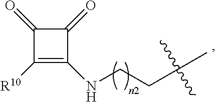

wherein

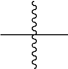

denotes a point of attachment;
wherein n is selected from 1, 2, 3, 4, and 5;
wherein R¹⁰ is —N(R)₂;
wherein each R is independently selected from C₁₋₆ alkyl, C₂₋₃ alkenyl, and H; and wherein n2 is selected from 1, 2, 3, 4, 5, 6, 7, 8, 9, and 10;
each R¹ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (XI):

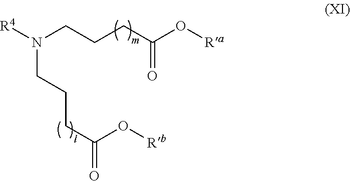

or an N-oxide or a salt thereof, wherein:
R′^{a} is R′^{branched} or R′^{cyclic};
R′^{branched} is:

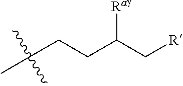

### and R′^{b} is:

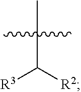

wherein

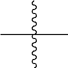

denotes a point of attachment;
R^{aγ} is selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

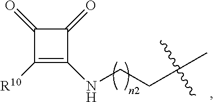

wherein

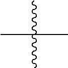

denotes a point of attachment;
wherein n is selected from 1, 2, 3, 4, and 5;
wherein R¹⁰ is —N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
R¹ is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (XI):

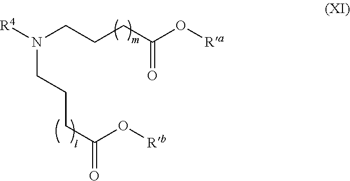

or an N-oxide or a salt thereof, wherein:
R′^{a} is R′^{branched} or R′^{cyclic};
R′^{branched} is:

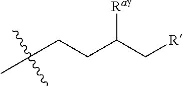

### and R′^{b} is:

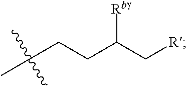

wherein

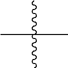

denotes a point of attachment;
R^{aγ} and R^{bγ} are each independently selected from C₁₋₁₂ alkyl and C₂₋₁₂ alkenyl;
R⁴ is selected from —(CH₂)ₙOH and

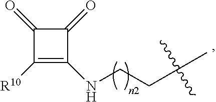

wherein

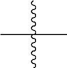

denotes a point of attachment;
wherein n is selected from 1, 2, 3, 4, and 5;
wherein R¹⁰ is —N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R¹ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (XI):

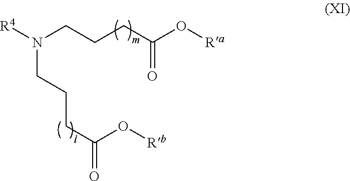

or an N-oxide or a salt thereof, wherein:
R′^{a} is R′^{branched} or R′^{cyclic}; wherein
R′^{branched} is:

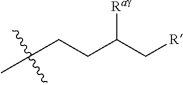

### and R′^{b} is:

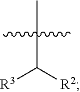

wherein

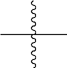

denotes a point of attachment;
R^{aγ} is selected from C₁₋₁₂ alkyl and C₂₋₁₂ alkenyl;
R² and R³ are each independently selected from C₁₋₁₄ alkyl and C₂₋₁₄ alkenyl;
R⁴ is —(CH₂)ₙOH wherein n is selected from 1, 2, 3, 4, and 5;
R¹ is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, m and l are each independently selected from 4, 5, and 6. In some embodiments m and l are each 5.

In some embodiments each R¹ independently is C₁₋₁₂ alkyl. In some embodiments, each R¹ independently is C₂₋₅ alkyl.

In some embodiments, R′^{b} is:

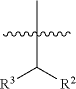

and R² and R³ are each independently C₁₋₁₄ alkyl.

In some embodiments, R′^{b} is:

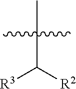

and R² and R³ are each independently C₆₋₁₀ alkyl.

In some embodiments, R′^{b} is:

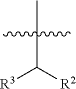

and R² and R³ are each C₈ alkyl.

In some embodiments, R′^{branched} is:

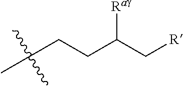

### and R′b is:

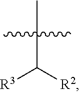

R^{aγ is} C₁₋₁₂ alkyl and R² and R³ are each independently C₆₋₁₀ alkyl.

In some embodiments, R′^{branched} is:

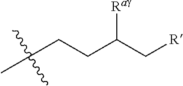

### and R′^{b} is:

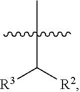

R^{aγ} is a C₂₋₆ alkyl and R² and R³ are each independently C₆₋₁₀ alkyl. In some embodiments, R′^{branched} is:

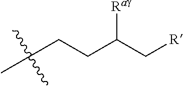

### and R′^{b} is:

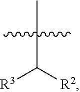

R^{aγ} is C₂₋₆ alkyl, and R² and R³ are each a C₈ alkyl.

In some embodiments, R′^{branched} is:

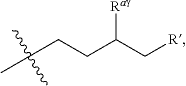

### R′^{b} is:

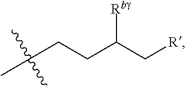

and R^{aγ} and R^{bγ} are each C₁₋₁₂ alkyl.

In some embodiments, R′^{branched} is:

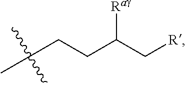

### R′^{b} is

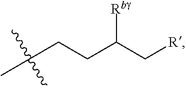

and R^{aγ} and R^{bγ} are each a C₂₋₆ alkyl.

In some embodiments, m and l are each independently selected from 4, 5, and 6 and each R¹ independently is C₁₋₁₂ alkyl. In some embodiments, m and l are each 5 and each R¹ independently is C₂₋₅ alkyl.

In some embodiments, R′^{branched} is:

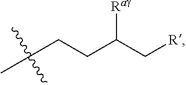

### R′^{b} is:

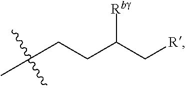

m and l are each independently selected from 4, 5, and 6, each R¹ independently is C₁₋₁₂ alkyl, and R^{aγ} and R^{bγ} are each C₁₋₁₂ alkyl.

In some embodiments, R′^{branched} is:

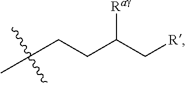

### R′^{b} is:

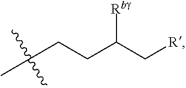

m and l are each 5, each R′ independently is a C₂₋₅ alkyl, and R^{aγ} and R^{bγ} are each a C₂₋₆ alkyl.

In some embodiments, R′^{branched} is:

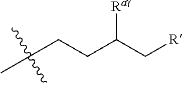

### and R′^{b} is:

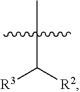

m and l are each independently selected from 4, 5, and 6, R′ is C₁₋₁₂ alkyl, R^{aγ} is C₁₋₁₂ alkyl and R² and R³ are each independently a C₆₋₁₀ alkyl.

In some embodiments, R′^{branched} is:

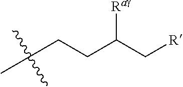

### and R′^{b} is:

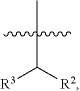

m and l are each 5, R′ is a C₂₋₅ alkyl, R^{aγ} is a C₂₋₆ alkyl, and R² and R³ are each a C₈ alkyl.

In some embodiments, R⁴ is

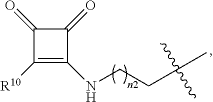

wherein R¹⁰ is —NH(C₁₋₆ alkyl) and n2 is 2.

In some embodiments, R⁴ is

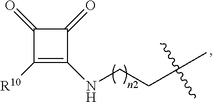

wherein R¹⁰ is —NH(CH₃) and n2 is 2.

In some embodiments, R′^{branched} is:

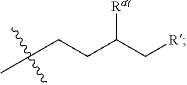

### R′^{b} is:

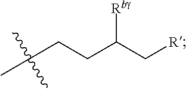

m and l are each independently selected from 4, 5, and 6; each R′ independently is C₁₋₁₂ alkyl; R^{aγ} and R^{bγ} are each C₁₋₁₂alkyl; and R⁴ is

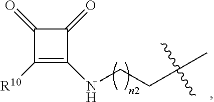

wherein R¹⁰ is —NH(C₁₋₆ alkyl), and n2 is 2.

In some embodiments, R′^{branched} is:

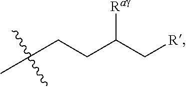

### R′^{b} is:

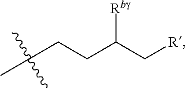

m and l are each 5, each R′ independently is a C₂₋₅ alkyl, R^{aγ} and R^{bγ} are each a C₂₋₆ alkyl, and R⁴ is

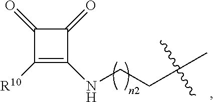

wherein R¹⁰ is —NH(CH₃) and n2 is 2.

In some embodiments, R′^{branched} is:

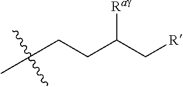

### and R′^{b} is:

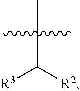

m and l are each independently selected from 4, 5, and 6, R′ is C₁₋₁₂ alkyl, R² and R³ are each independently a C₆₋₁₀ alkyl, R^{aγ} is C₁₋₁₂ alkyl, and R⁴ is

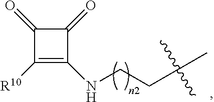

wherein R¹⁰ is —NH(C₁₋₆ alkyl) and n2 is 2.

In some embodiments, R′^{branched} is:

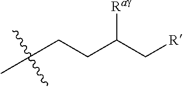

### and R′^{b} is:

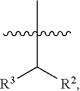

m and l are each 5, R′ is a C₂₋₅ alkyl, R^{aγ} is a C₂₋₆ alkyl, R² and R³ are each a C₈ alkyl, and R⁴ is

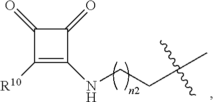

wherein R¹⁰ is —NH(CH₃) and n2 is 2.

In some embodiments, R⁴ is —(CH₂)ₙOH and n is 2, 3, or 4. In some embodiments, R⁴ is —(CH₂)ₙOH and n is 2.

In some embodiments, R′^{branched} is

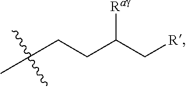

### R′^{b} is:

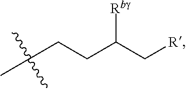

m and l are each independently selected from 4, 5, and 6, each R¹ independently is C₁₋₁₂ alkyl, R^{aγ} and R^{bγ} are each C₁₋₁₂alkyl, R⁴ is —(CH₂)ₙOH, and n is 2, 3, or 4.

In some embodiments, R′^{branched} is:

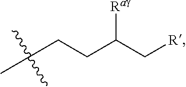

### R′^{b} is:

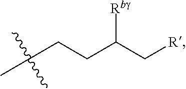

m and l are each 5, each R′ independently is a C₂₋₅ alkyl, R^{aγ} and R^{bγ} are each a C₂₋₅alkyl, R⁴ is —(CH₂)ₙOH, and n is 2.

In some embodiments, the ionizable lipid is a compound of Formula (XI):

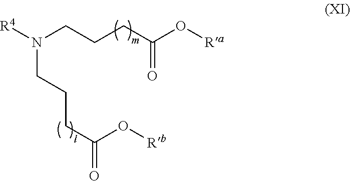

or an N-oxide or a salt thereof, wherein:
R′^{a} is R′^{branched} or R′^{cyclic}; wherein
R′^{branched} is:

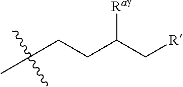

### and R′^{b} is:

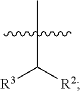

wherein

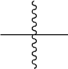

denotes a point of attachment;
R^{aγ} is C₁₋₁₂ alkyl;
R² and R³ are each independently C₁₋₁₄ alkyl;
R⁴ is —(CH₂)ₙOH wherein n is selected from 1, 2, 3, 4, and 5;
R′ is C₁₋₁₂ alkyl;
m is selected from 4, 5, and 6; and
l is selected from 4, 5, and 6.

In some embodiments, m and l are each 5, and n is 2, 3, or 4.

In some embodiments, R′ is a C₂₋₅ alkyl, R^{aγ} is a C₂₋₆ alkyl, and R² and R³ are each C₆₋₁₀ alkyl.

In some embodiments, m and l are each 5, n is 2, 3, or 4, R¹ is a C₂₋₅ alkyl, R^{aγ} is C₂₋₆ alkyl, and R² and R³ are each a C₆₋₁₀ alkyl.

In some embodiments, the ionizable lipid is a compound of Formula (XI-g):

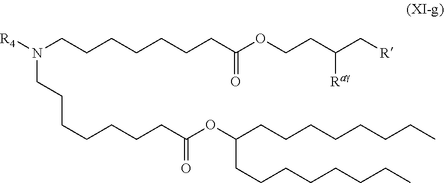

or an N-oxide or salt thereof, wherein:
R^{aγ} is C₂₋₆ alkyl;
R′ is C₂₋₅ alkyl; and
R⁴ is selected from —(CH₂)ₙOH and

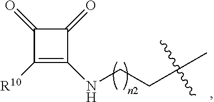

wherein

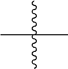

denotes a point of attachment,
wherein n is selected from 3, 4, and 5; and
wherein R¹⁰ is —NH(C₁₋₆ alkyl); and
wherein n2 is selected from 1, 2, and 3.

In some embodiments, the ionizable lipid is a compound of Formula (XI-h):

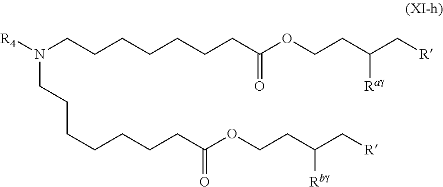

or an N-oxide or salt thereof, wherein:
R^{aγ} and R^{bγ} are each independently a C₂₋₆ alkyl;
each R′ independently is a C₂₋₅ alkyl; and
R⁴ is selected from —(CH₂)ₙOH and

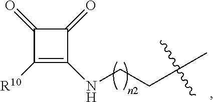

wherein

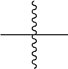

denotes a point of attachment,
wherein n is selected from 3, 4, and 5;
wherein R¹⁰ is —NH(C₁₋₆ alkyl); and
wherein and n2 is selected from 1, 2, and 3.

In some embodiments, R⁴ is

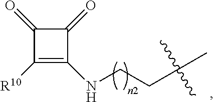

wherein R¹⁰ is —NH(CH₃) and n2 is 2.

In some embodiments, R⁴ is —(CH₂)₂OH.

In some embodiments, the ionizable lipid is a compound having Formula (XII):

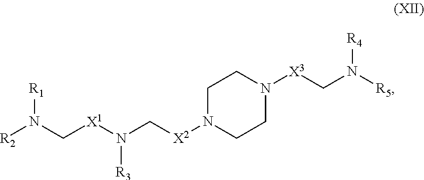

or an N-oxide or a salt thereof, wherein:
R¹, R², R³, R⁴, and R⁵ are independently selected from C₅₋₂₀ alkyl, C₅₋₂₀ alkenyl, —R″MR′, —R*YR″, —YR″, and —R*OR″;
each M is independently selected
from —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are each independently selected from a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from C₁₋₁₂ alkyl and C₂₋₁₂ alkenyl;
each R is independently selected from C₁₋₃ alkyl and a C₃₋₆ carbocycle;
each R′ is independently selected from C₁₋₁₂ alkyl, C₂₋₁₂ alkenyl, and H; and
each R″ is independently selected from C₃₋₁₂ alkyl and C₃₋₁₂ alkenyl, and wherein:
i) at least one of X¹, X², and X³ is not —CH₂—; and/or
ii) at least one of R¹, R², R³, R⁴, and R⁵ is —R″MR′.

In some embodiments, R¹, R², R³, R⁴, and R⁵ are each C₅₋₂₀ alkyl; X¹ is —CH₂—; and X² and X³ are each —C(O)—.

In some embodiments, the compound of Formula (XII) is:

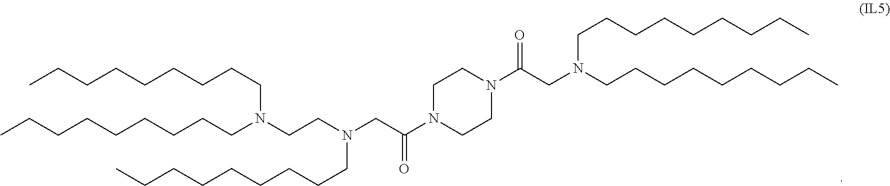 [SMILES: CCCCCCCCCN(CCCCCCCCC)CCN(CCCCCCCCC)CC(=O)N1CCN(C(=O)CN(CCCCCCCCC)CCCCCCCCC)CC1]

In some embodiments, a lipid nanoparticle composition includes a lipid component comprising a compound as described herein (e.g., a compound according to Formula (I), (IA), (II), (IIa), (IIb), (IIc), (IId), (IIe), (X), (XI), (XI-g), (XI-h), or (XII)).

In some embodiments LNPs may be comprised of ionizable lipids including a central piperazine moiety. Such LNPs advantageously may be composed of an ionizable lipid, a phospholipid and a PEG lipid and may optionally include a structural lipid or may lack a structural lipid. In some embodiments the phospholipid is a DSPC or DOP.

The ionizable lipids including a central piperazine moiety described herein may be advantageously used in lipid nanoparticle compositions for the delivery of therapeutic and/or prophylactic agents to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent has an increased therapeutic index as compared to a corresponding formulation which comprises a reference lipid (e.g., MC3, KC2, or DLinDMA) and the same therapeutic or prophylactic agent.

Lipids may be compounds of Formula (III),

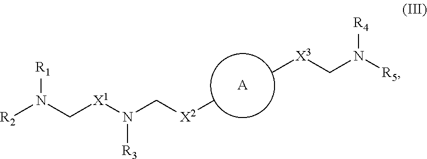

or salts or isomers thereof, wherein
ring A is

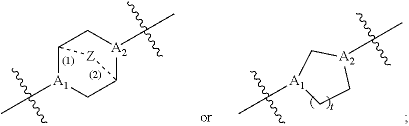

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R¹, R², R³, R⁴, and R⁵ are independently selected from the group consisting of C₅₋₂₀ alkyl, C₅₋₂₀alkenyl, —R″MR′, —R*YR″, —YR″, and —R*OR″;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, —(CH₂)₂, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R¹ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl,
wherein when ring A is

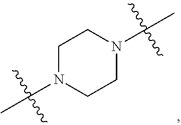 [SMILES: *N1CCN(*)CC1]

then
i) at least one of X¹, X², and X³ is not —CH₂—; and/or
ii) at least one of R¹, R², R³, R⁴, and R⁵ is —R″MR′.

In some embodiments, the compound is of any of formulae (IIIa1)-(IIIa6):

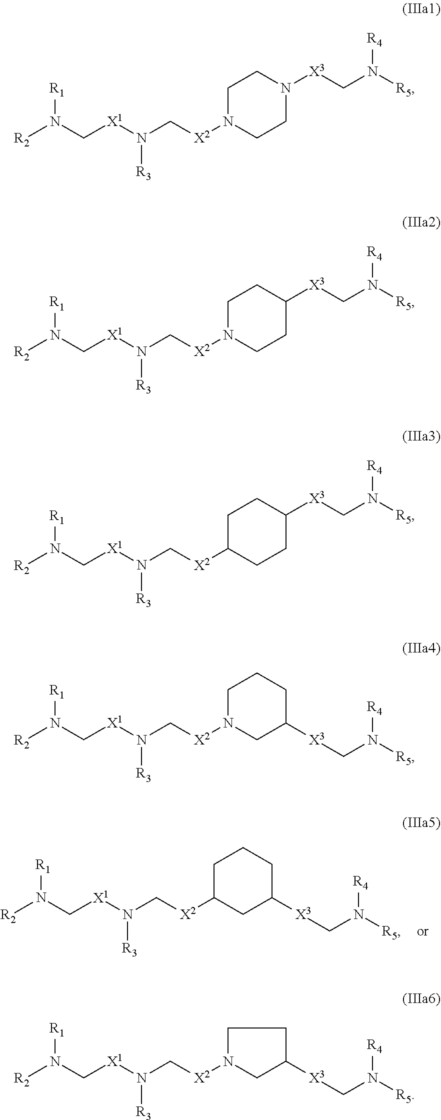

The compounds of Formula (III) or any of (IIIa1)-(IIIa6) include one or more of the following features when applicable.

In some embodiments, ring A is

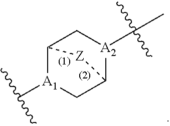

In some embodiments, ring A is

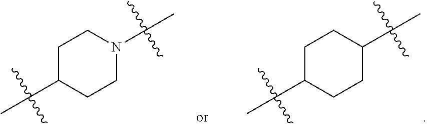 [SMILES: *C1CCN(*)CC1 ; *C1CCC(*)CC1]

In some embodiments, ring A is

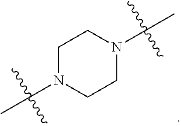 [SMILES: *N1CCN(*)CC1]

In some embodiments, ring A is

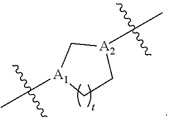

In some embodiments, ring A is

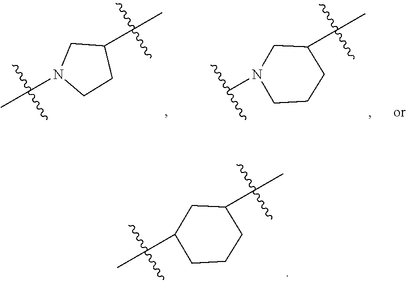 [SMILES: *C1CCN(*)C1 ; *C1CCCN(*)C1 ; *C1CCCC(*)C1]

In some embodiments, ring A is

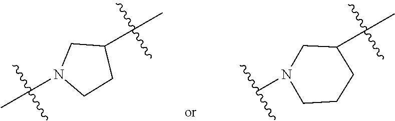 [SMILES: *C1CCN(*)C1 ; *C1CCCN(*)C1]

wherein ring, in which the N atom is connected with X².

In some embodiments, Z is CH₂.

In some embodiments, Z is absent.

In some embodiments, at least one of A₁ and A₂ is N.

In some embodiments, each of A₁ and A₂ is N.

In some embodiments, each of A₁ and A₂ is CH.

In some embodiments, A₁ is N and A₂ is CH.

In some embodiments, A₁ is CH and A₂ is N.

In some embodiments, at least one of X¹, X², and X³ is not —CH₂—. For example, in certain embodiments, X¹ is not —CH₂—. In some embodiments, at least one of X¹, X², and X³ is —C(O)—.

In some embodiments, X² is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—.

In some embodiments, X³ is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—. In other embodiments, X³ is —CH₂—.

In some embodiments, X³ is a bond or —(CH₂)₂—.

In some embodiments, R¹ and R² are the same. In certain embodiments, R¹, R², and R³ are the same.

In some embodiments, R⁴ and R⁵ are the same. In certain embodiments, R¹, R², R³, R⁴, and R⁵ are the same.

In some embodiments, at least one of R¹, R², R³, R⁴, and R⁵ is —R″MR′. In some embodiments, at most one of R¹, R², R³, R⁴, and R⁵ is —R″MR′. For example, at least one of R¹, R², and R³ may be —R″MR′, and/or at least one of R⁴ and R⁵ is —R″MR′. In certain embodiments, at least one M is —C(O)O—. In some embodiments, each M is —C(O)O—. In some embodiments, at least one M is —OC(O)—. In some embodiments, each M is —OC(O)—. In some embodiments, at least one M is —OC(O)O—. In some embodiments, each M is —OC(O)O—. In some embodiments, at least one R″ is C₃ alkyl. In certain embodiments, each R″ is C₃ alkyl. In some embodiments, at least one R″ is C₅ alkyl. In certain embodiments, each R″ is C₅₋alkyl. In some embodiments, at least one R″ is C₆ alkyl. In certain embodiments, each R″ is C₆ alkyl. In some embodiments, at least one R″ is C₇ alkyl. In certain embodiments, each R″ is C₇ alkyl. In some embodiments, at least one R′ is C₅ alkyl. In certain embodiments, each R′ is C₅₋alkyl. In other embodiments, at least one R′ is C₁ alkyl. In certain embodiments, each R′ is C₁ alkyl. In some embodiments, at least one R′ is C₂ alkyl. In certain embodiments, each R′ is C₂ alkyl.

In some embodiments, at least one of R₁, R₂, R₃, R₄, and R₅ is C₁₂ alkyl. In certain embodiments, each of R₁, R₂, R₃, R₄, and R₅ are C₁₂ alkyl.

In certain embodiments, the compound is selected from the group consisting of:

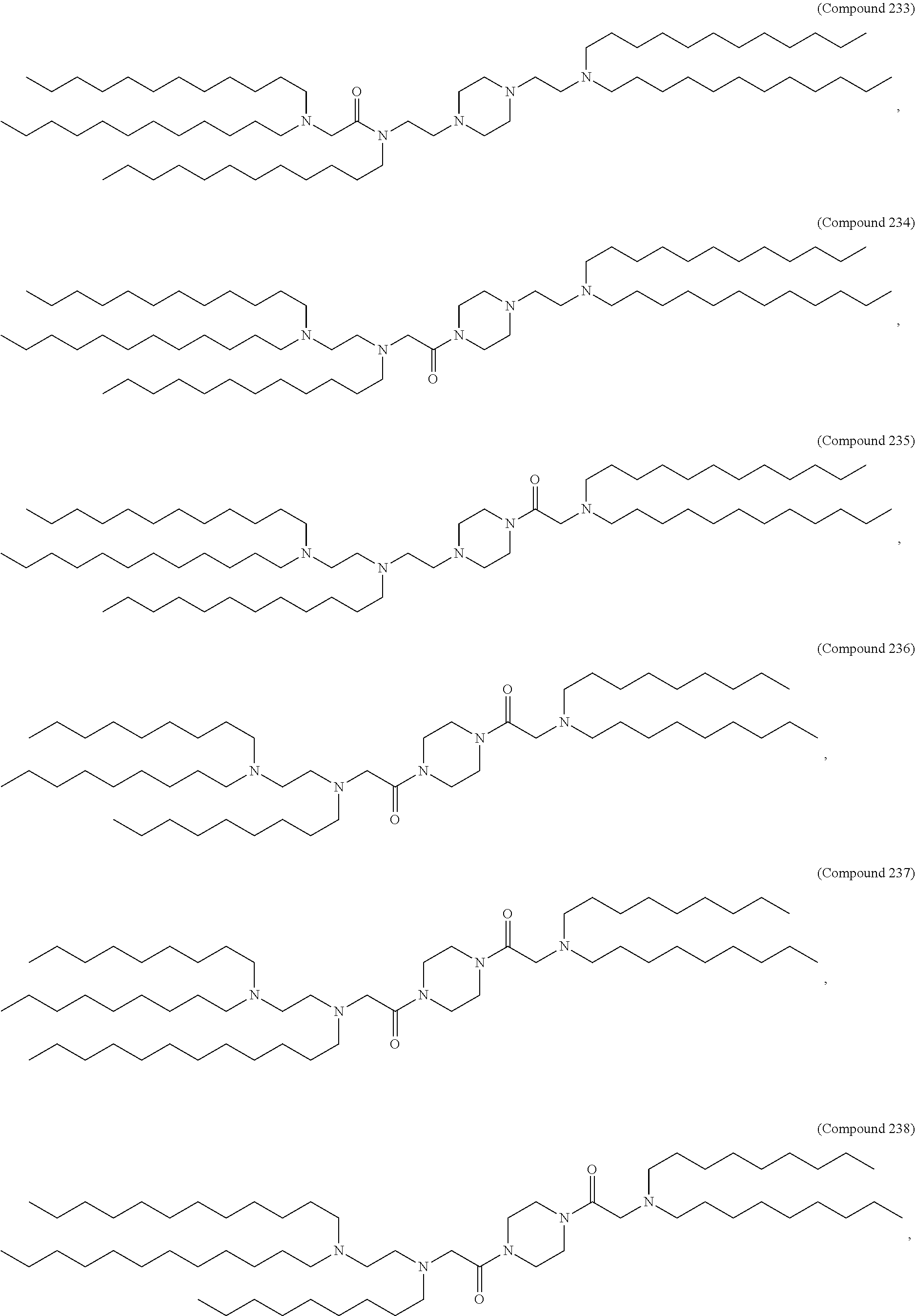

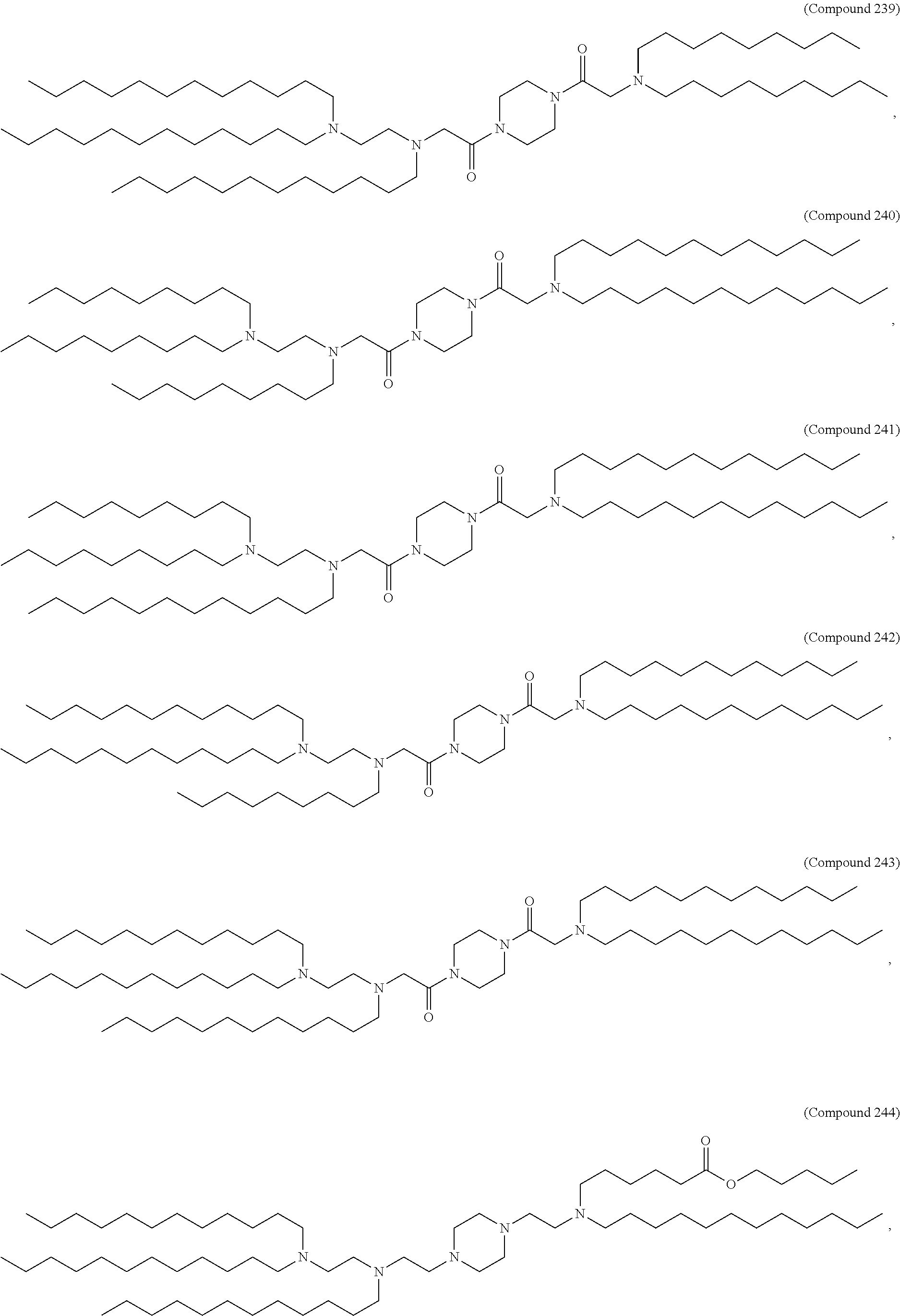

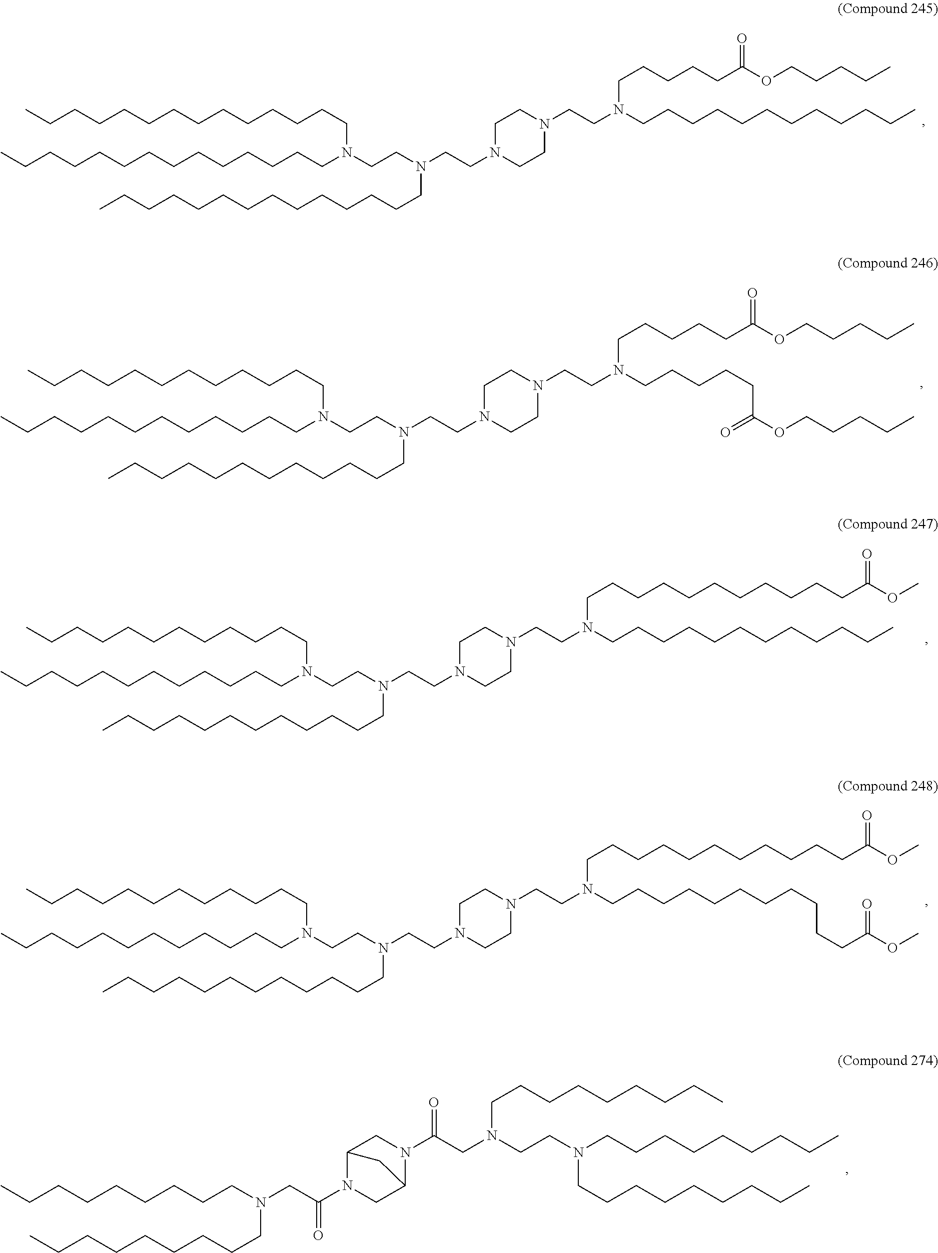

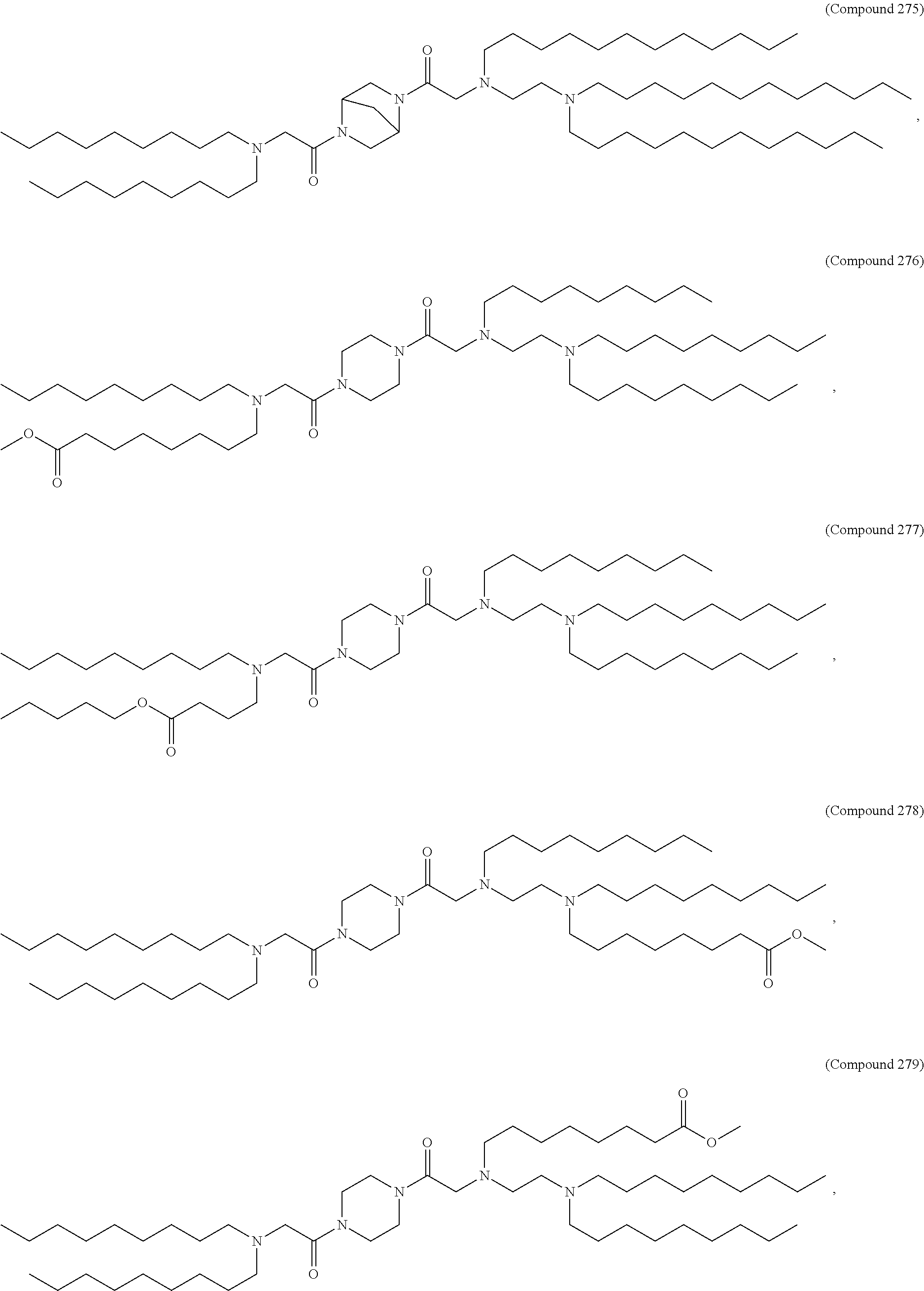

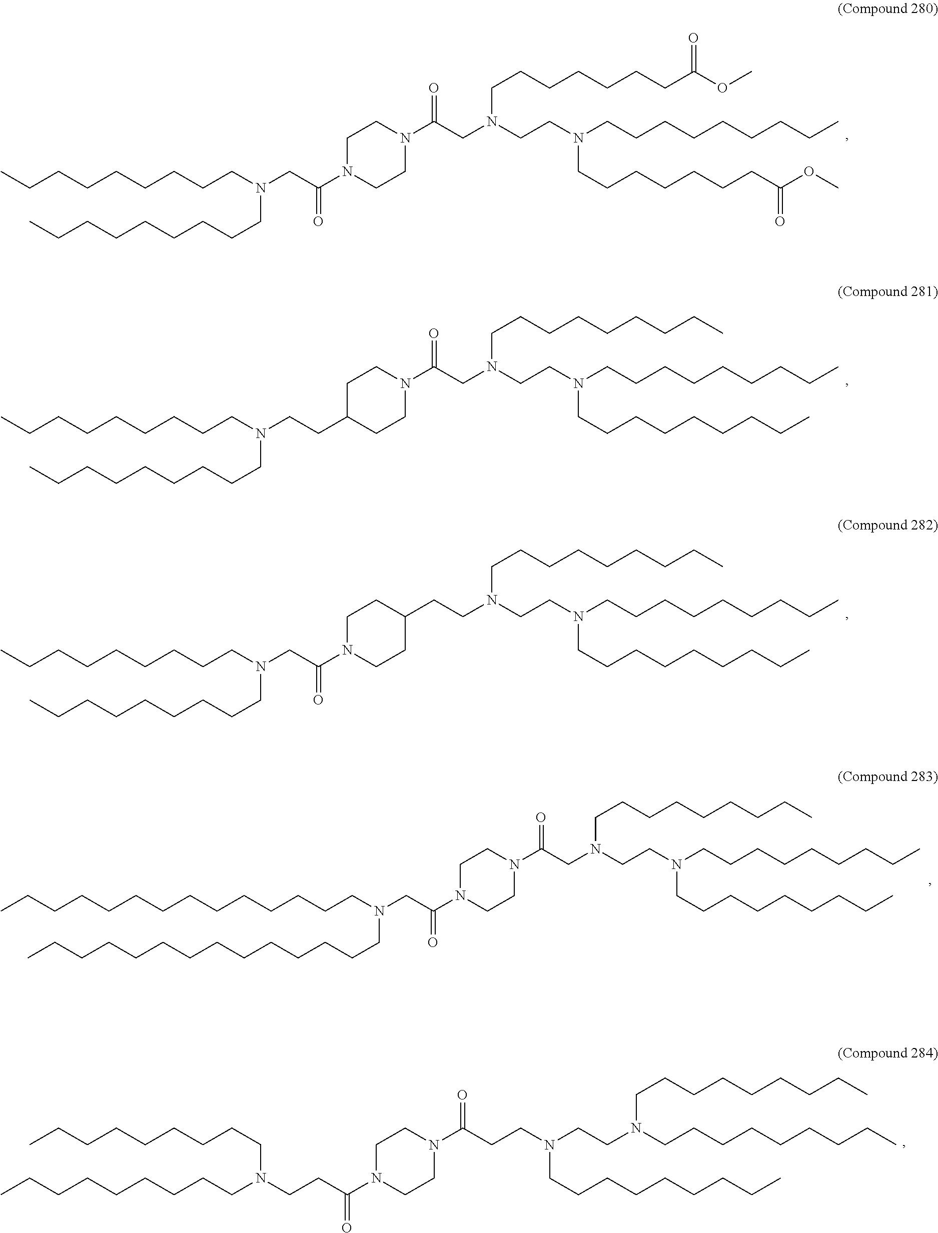

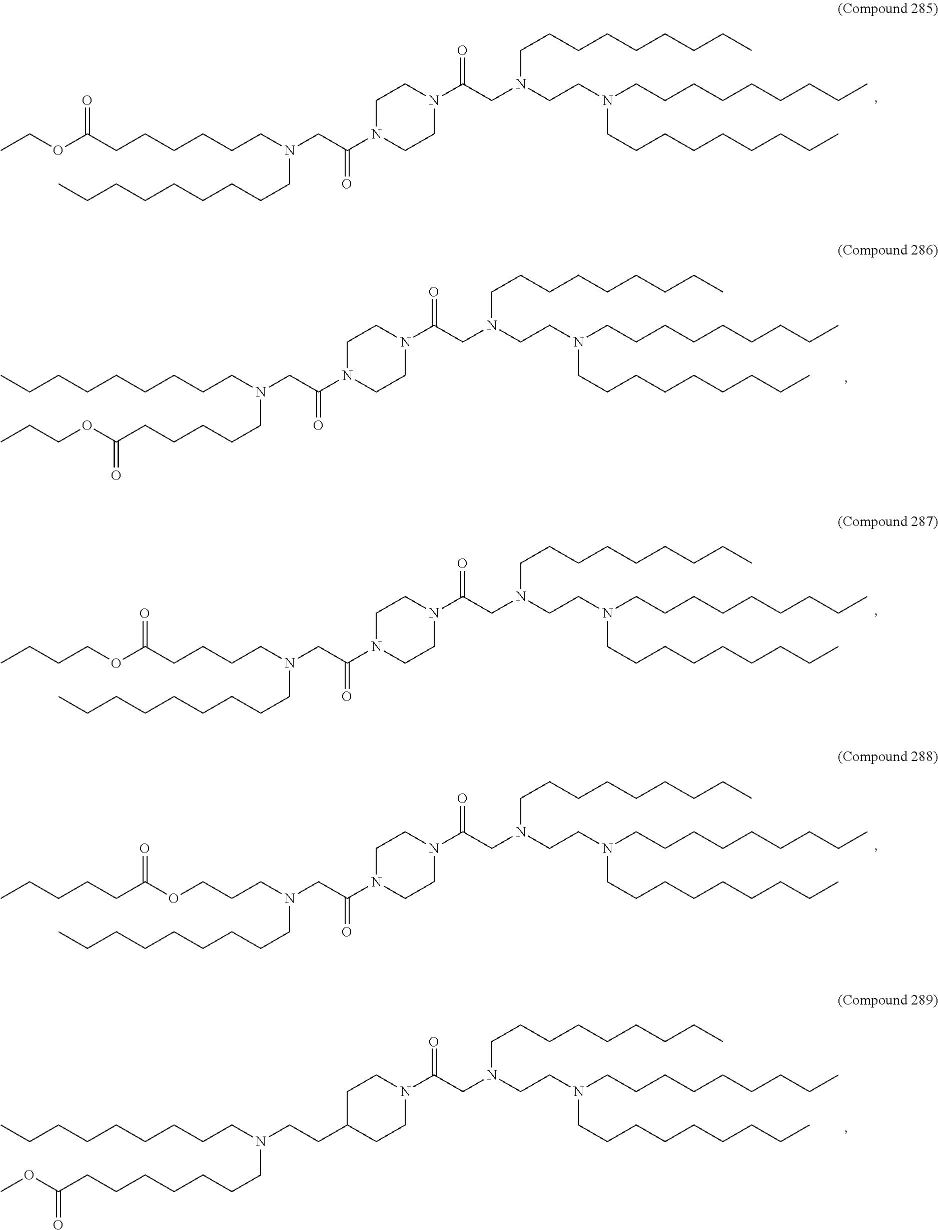

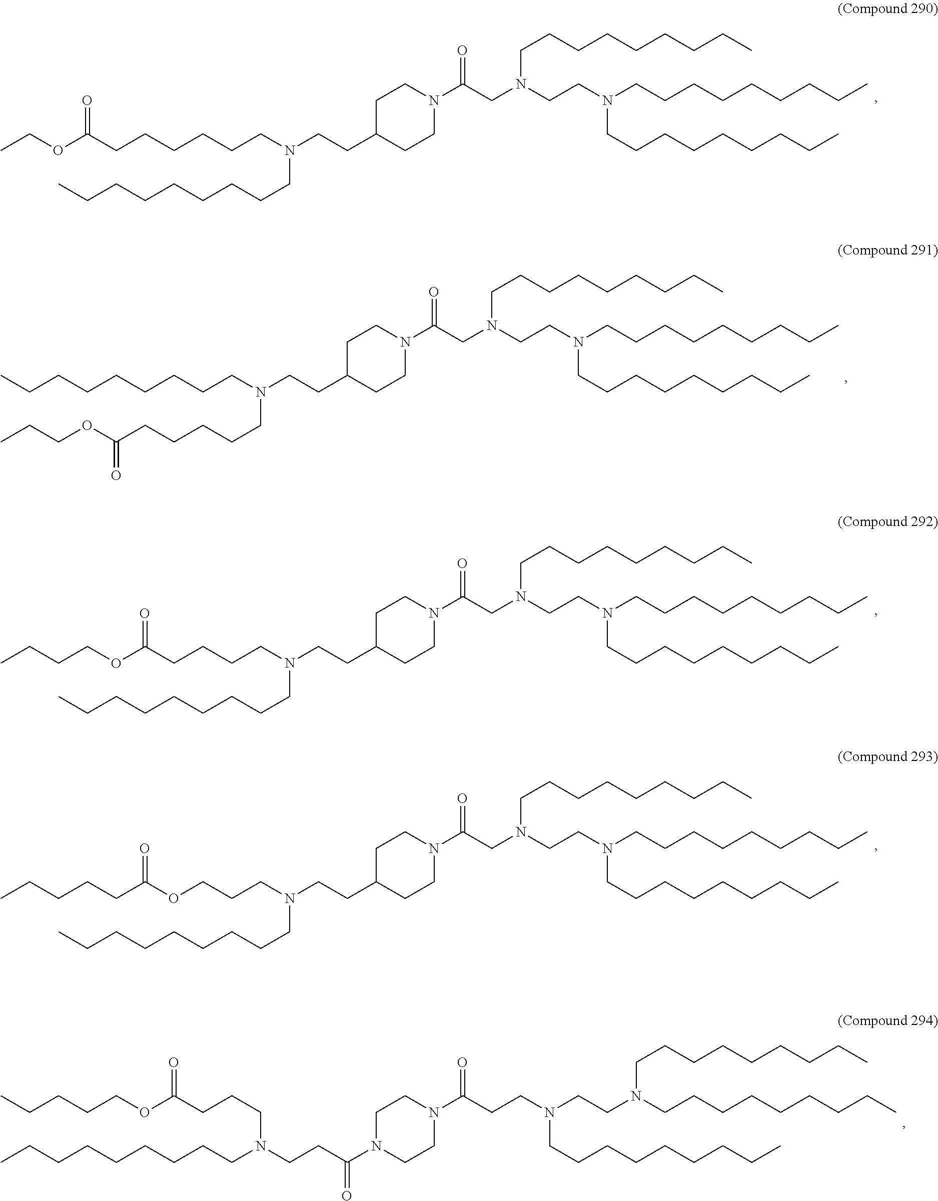

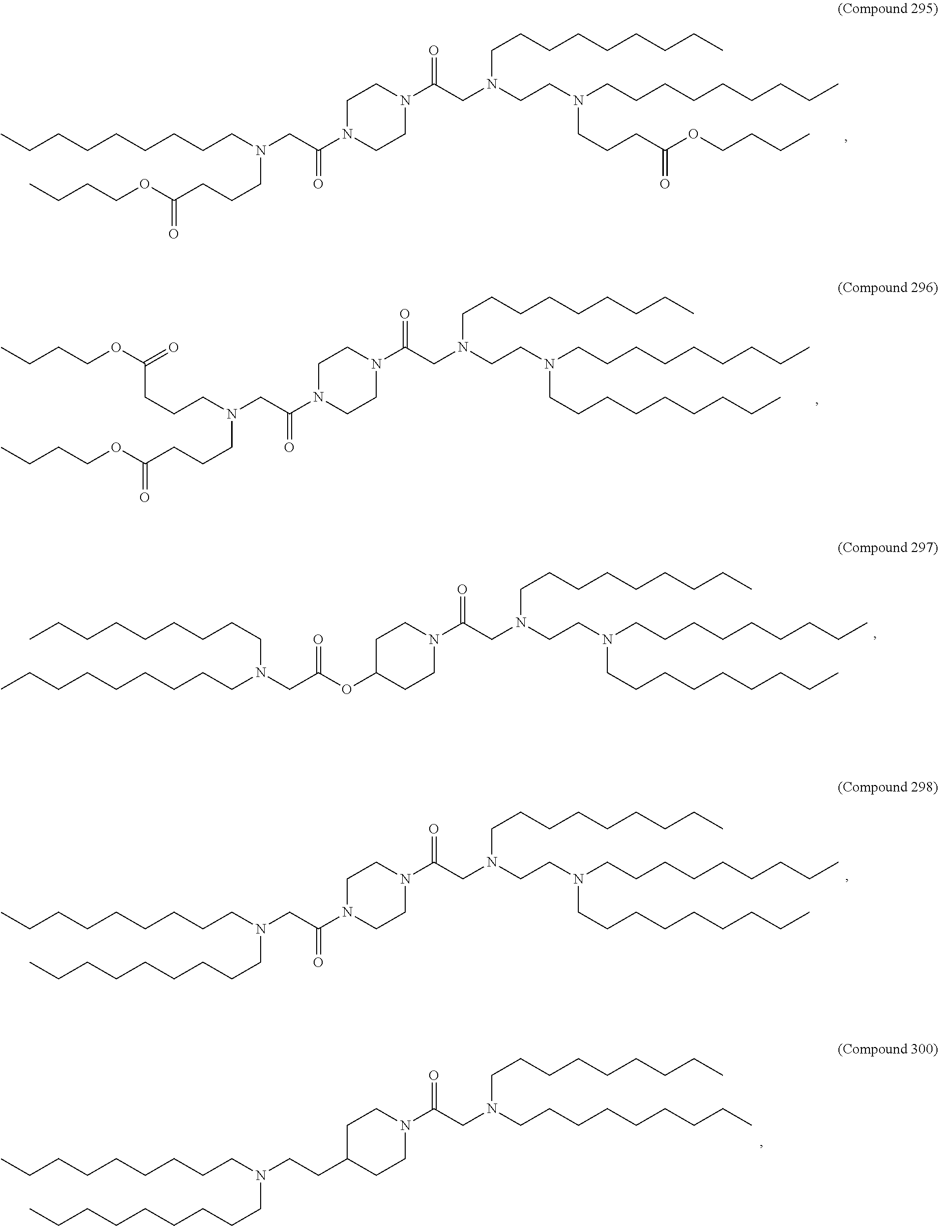

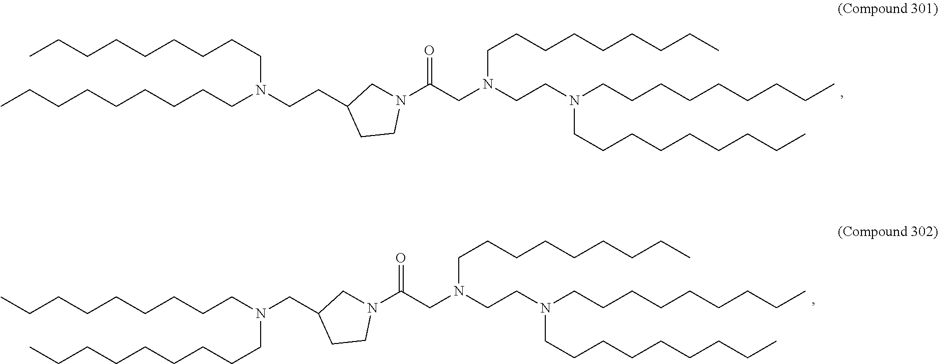

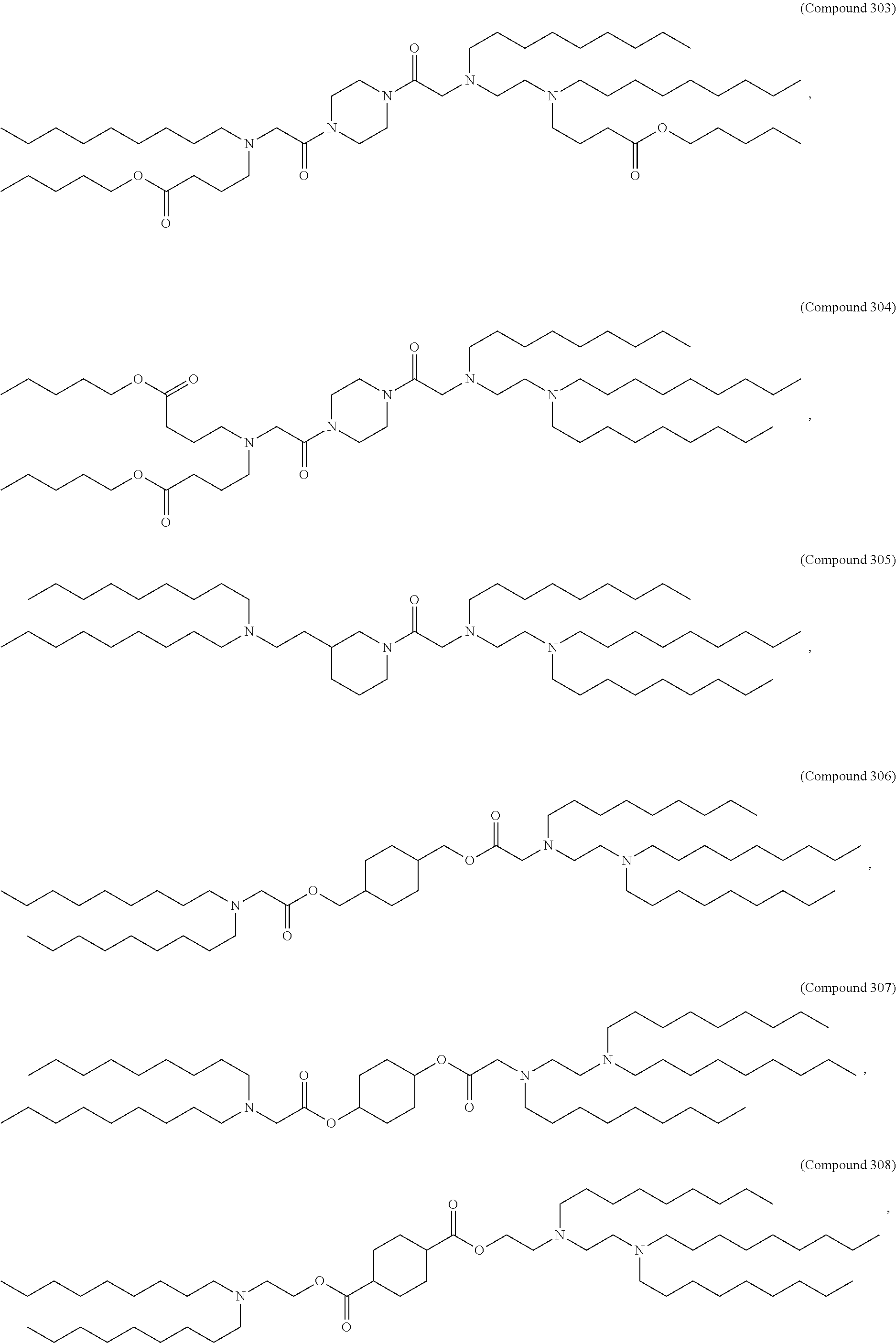

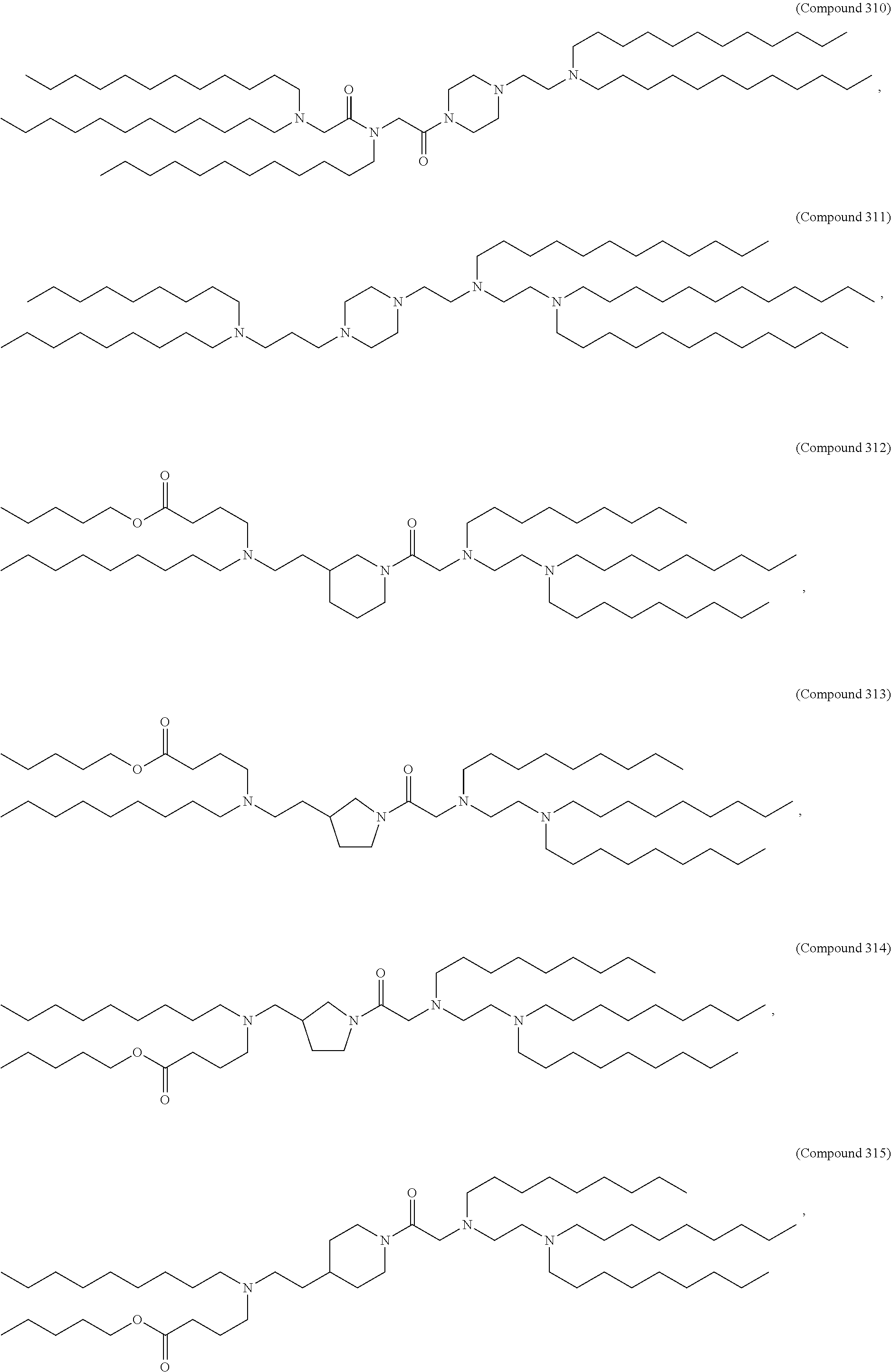

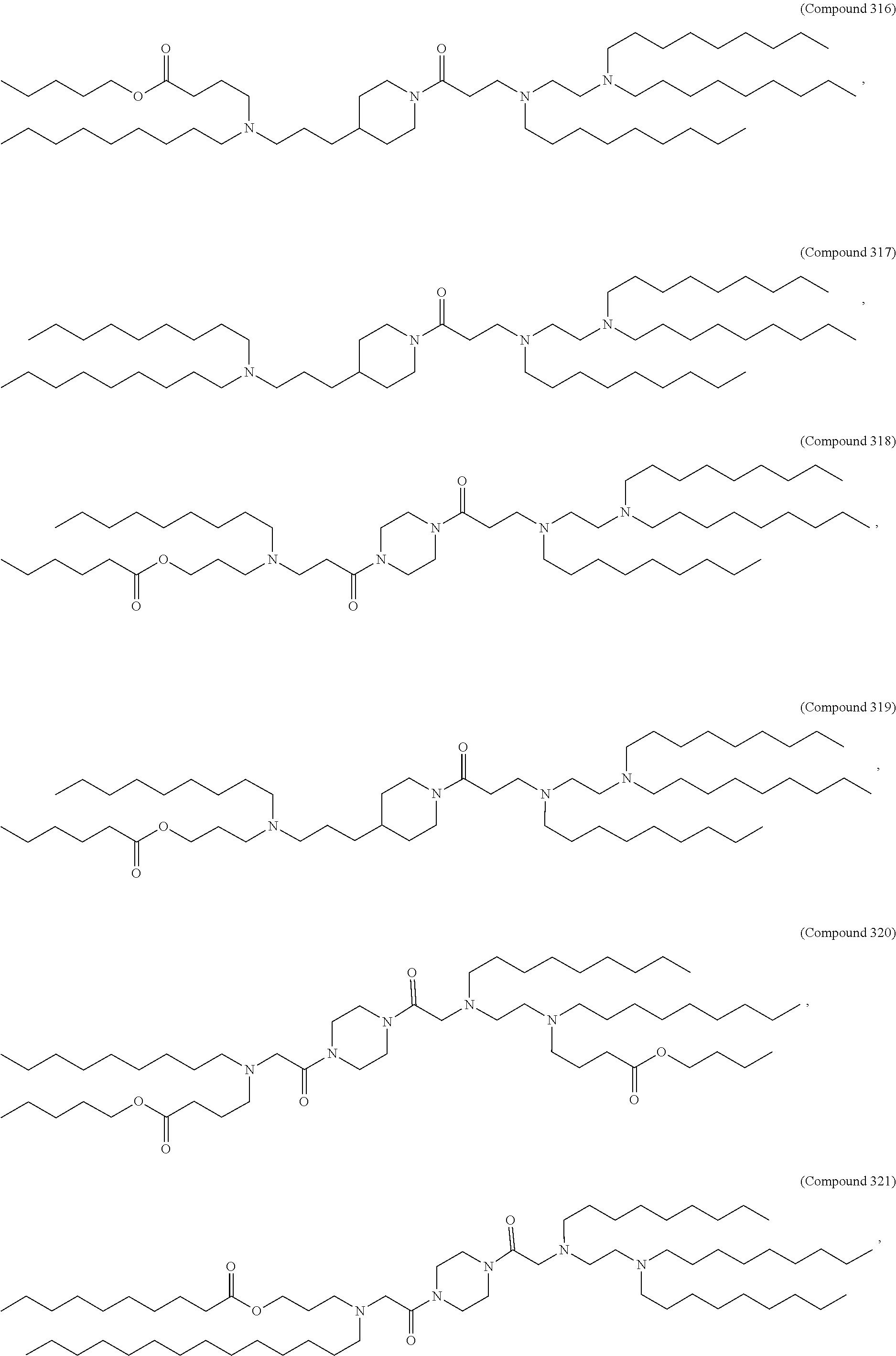

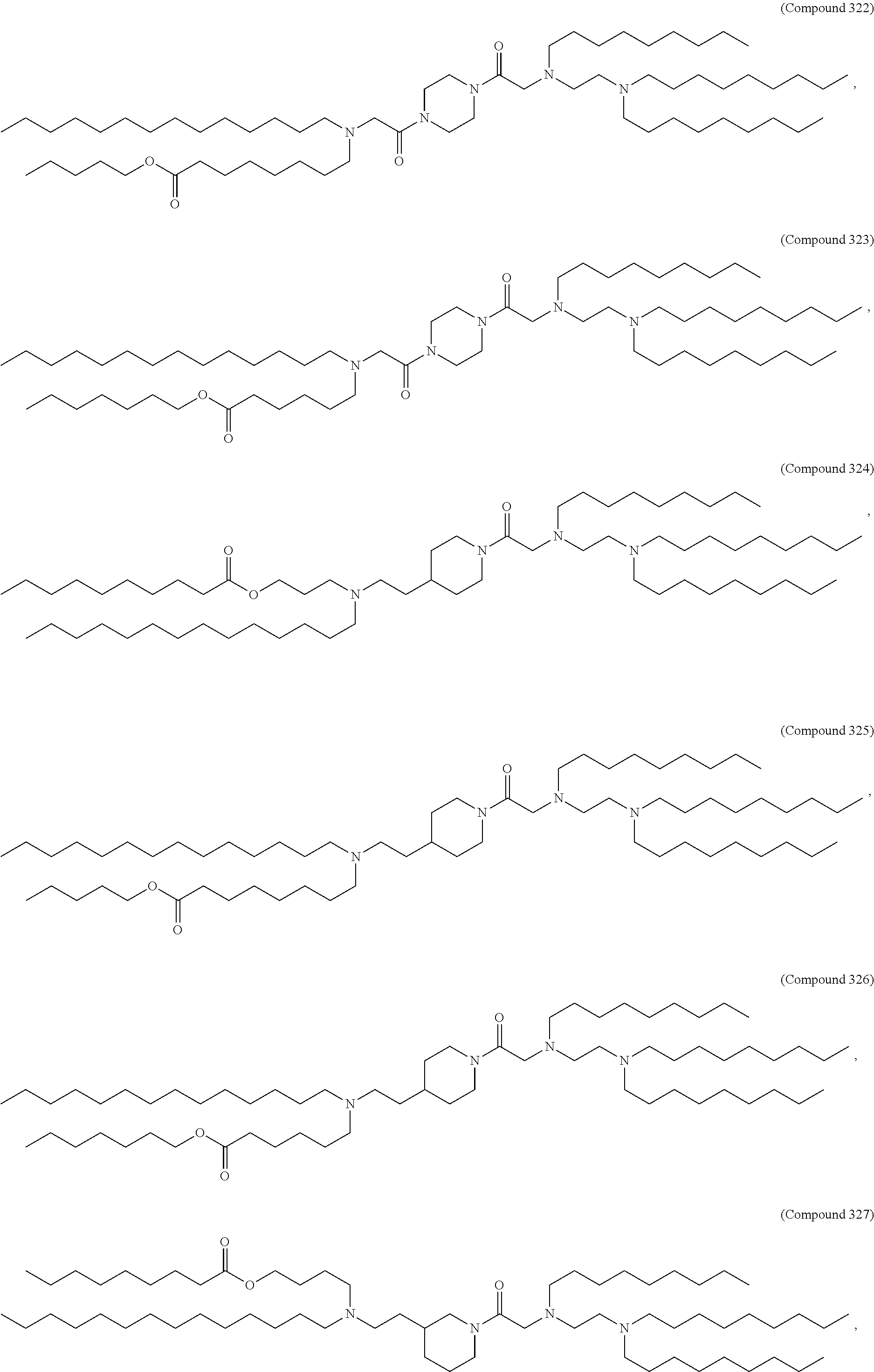

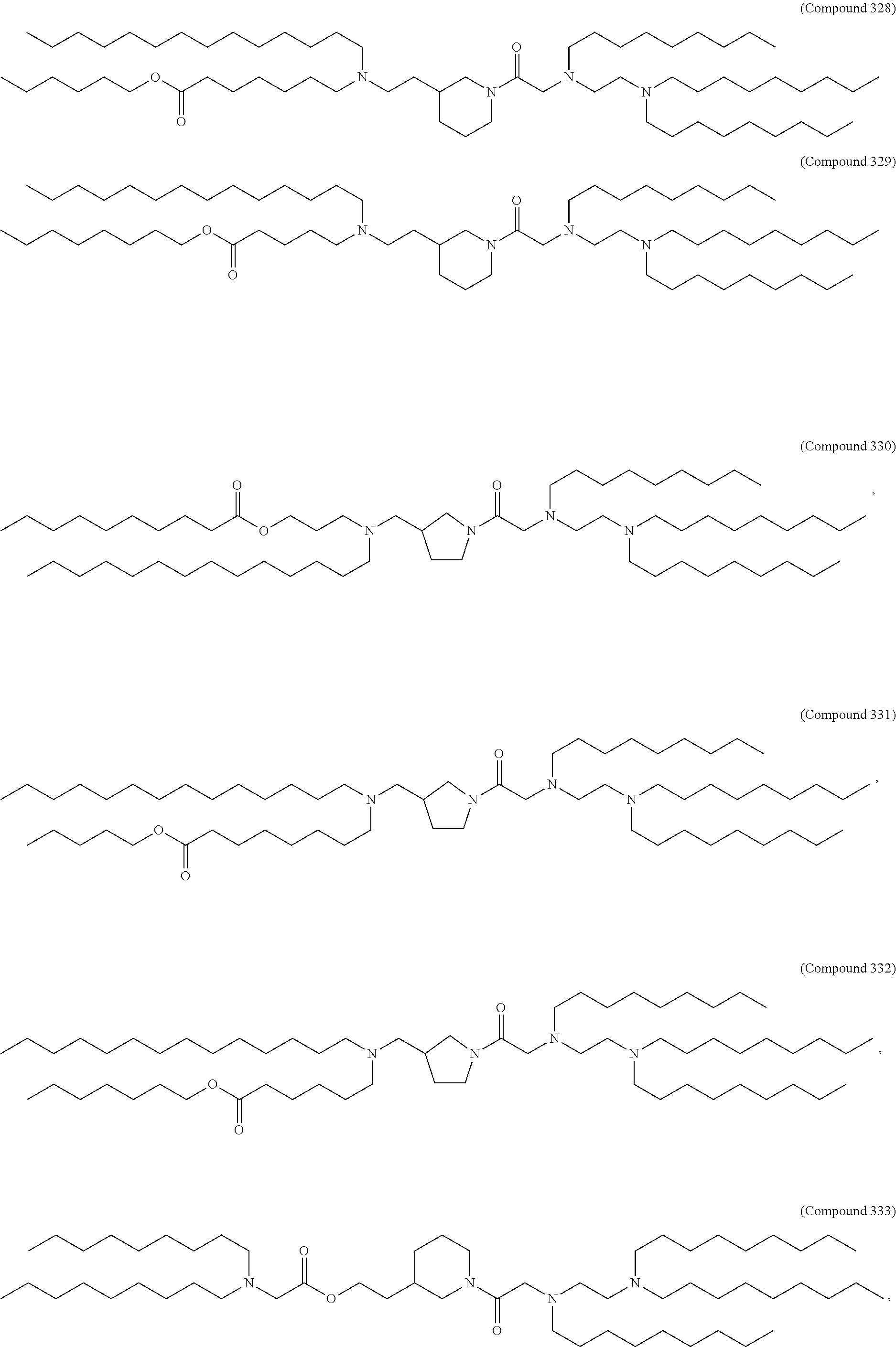

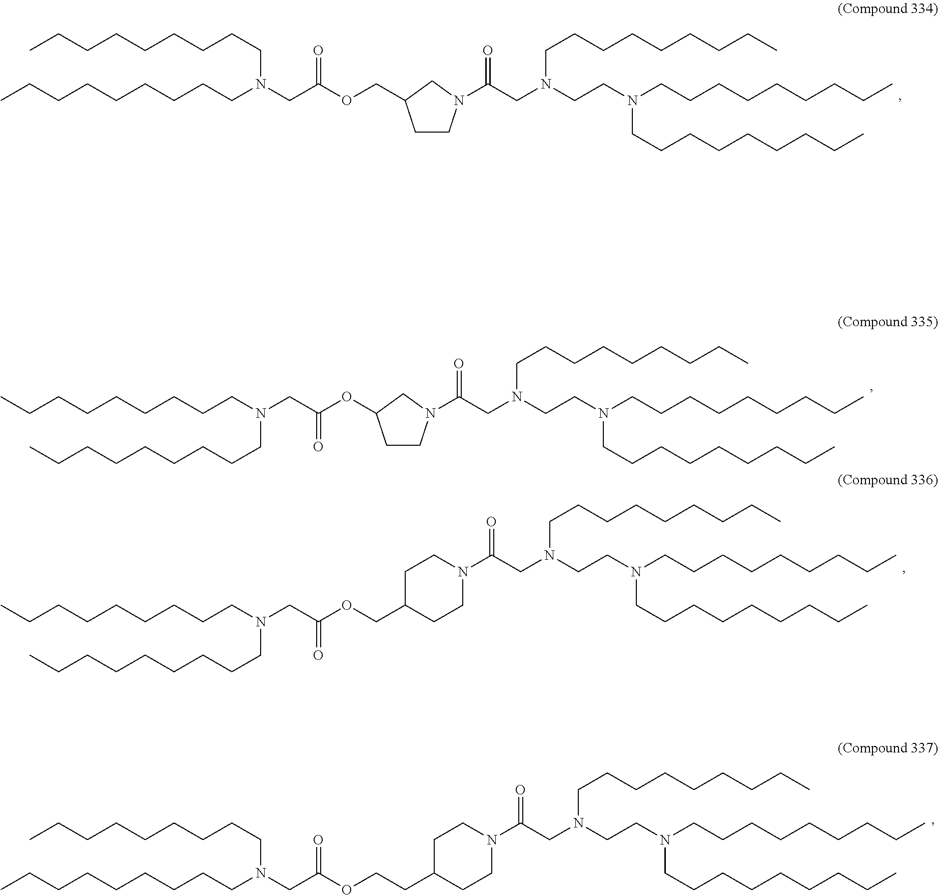

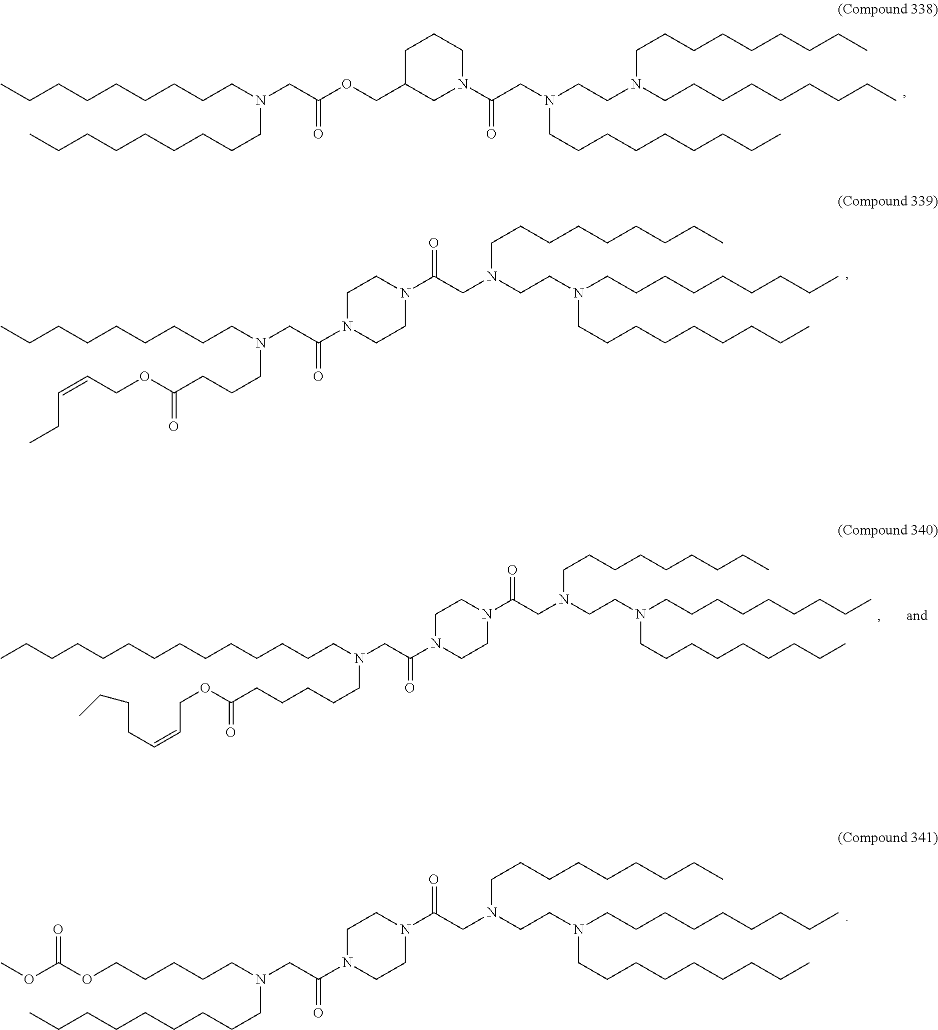

In other embodiments, a lipid has the Formula (IV)

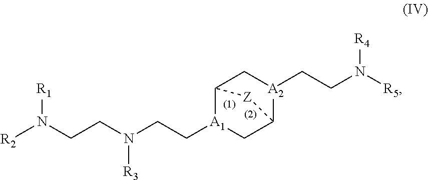

or a salt or isomer thereof, wherein
A₁ and A₂ are each independently selected from CH or N and at least one of A₁ and A₂ is N;
Z is CH₂ or absent wherein when Z is —CH₂—, the dashed lines (1) and (2) each represent a single bond; and when Z is absent, the dashed lines (1) and (2) are both absent;
R₁, R₂, R₃, R₄, and R₅ are independently selected from the group consisting of C₆₋₂₀ alkyl and C₆₋₂₀ alkenyl;
wherein when ring A is

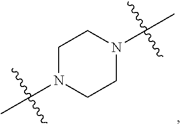 [SMILES: *N1CCN(*)CC1]

then
i) R¹, R², R³, R⁴, and R⁵ are the same, wherein R¹ is not C₁₂ alkyl, C₁₈ alkyl, or C₁₈ alkenyl;
ii) only one of R¹, R², R³, R⁴, and R⁵ is selected from C₆₋₂₀ alkenyl;
iii) at least one of R¹, R², R³, R⁴, and R⁵ have a different number of carbon atoms than at least one other of R¹, R², R³, R⁴, and R⁵;
iv) R¹, R², and R³ are selected from C₆₋₂₀ alkenyl, and R⁴ and R⁵ are selected from C₆₋₂₀ alkyl; or
v) R¹, R², and R³ are selected from C₆₋₂₀ alkyl, and R⁴ and R⁵ are selected from C₆₋₂₀ alkenyl.

In some embodiments, the compound is of Formula (IVa):

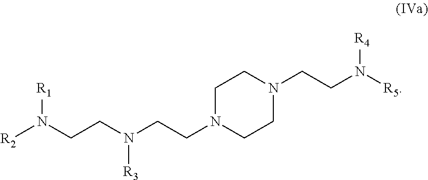

The compounds of Formula (IV) or (IVa) include one or more of the following features when applicable.

In some embodiments, Z is —CH₂—.

In some embodiments, Z is absent.

In some embodiments, at least one of A₁ and A₂ is N.

In some embodiments, each of A₁ and A₂ is N.

In some embodiments, each of A₁ and A₂ is CH.

In some embodiments, A₁ is N and A₂ is CH.

In some embodiments, A₁ is CH and A₂ is N.

In some embodiments, R₁, R₂, R₃, R₄, and R₅ are the same, and are not C₁₂ alkyl, C₁₈ alkyl, or C₁₈ alkenyl. In some embodiments, R₁, R₂, R₃, R₄, and R₅ are the same and are C₉ alkyl or C₁₄ alkyl.

In some embodiments, only one of R₁, R₂, R₃, R₄, and R₅ is selected from C₆₋₂₀ alkenyl. In certain such embodiments, R₁, R₂, R₃, R₄, and R₅ have the same number of carbon atoms. In some embodiments, R₄ is selected from C₅₋₂₀ alkenyl. For example, R₄ may be C₁₂ alkenyl or Cis alkenyl.

In some embodiments, at least one of R₁, R₂, R₃, R₄, and R₅ have a different number of carbon atoms than at least one other of R₁, R₂, R₃, R₄, and R₅.

In certain embodiments, R₁, R₂, and R₃ are selected from C₆₋₂₀ alkenyl, and R₄ and R₅ are selected from C₆₋₂₀ alkyl. In other embodiments, R₁, R₂, and R₃ are selected from C₆₋₂₀ alkyl, and R₄ and R₅ are selected from C₆₋₂₀ alkenyl. In some embodiments, R₁, R₂, and R₃ have the same number of carbon atoms, and/or R₄ and R₅ have the same number of carbon atoms. For example, R₁, R₂, and R₃, or R₄ and R₅, may have 6, 8, 9, 12, 14, or 18 carbon atoms. In some embodiments, R₁, R₂, and R₃, or R₄ and R₅, are C₁₈ alkenyl (e.g., linoleyl). In some embodiments, R₁, R₂, and R₃, or R₄ and R₅, are alkyl groups including 6, 8, 9, 12, or 14 carbon atoms.

In some embodiments, R₁ has a different number of carbon atoms than R₂, R₃, R₄, and R₅. In other embodiments, R3 has a different number of carbon atoms than R₁, R₂, R₄, and R₅. In further embodiments, R4 has a different number of carbon atoms than R₁, R₂, R₃, and R₅.

In some embodiments, the compound is selected from the group consisting of:

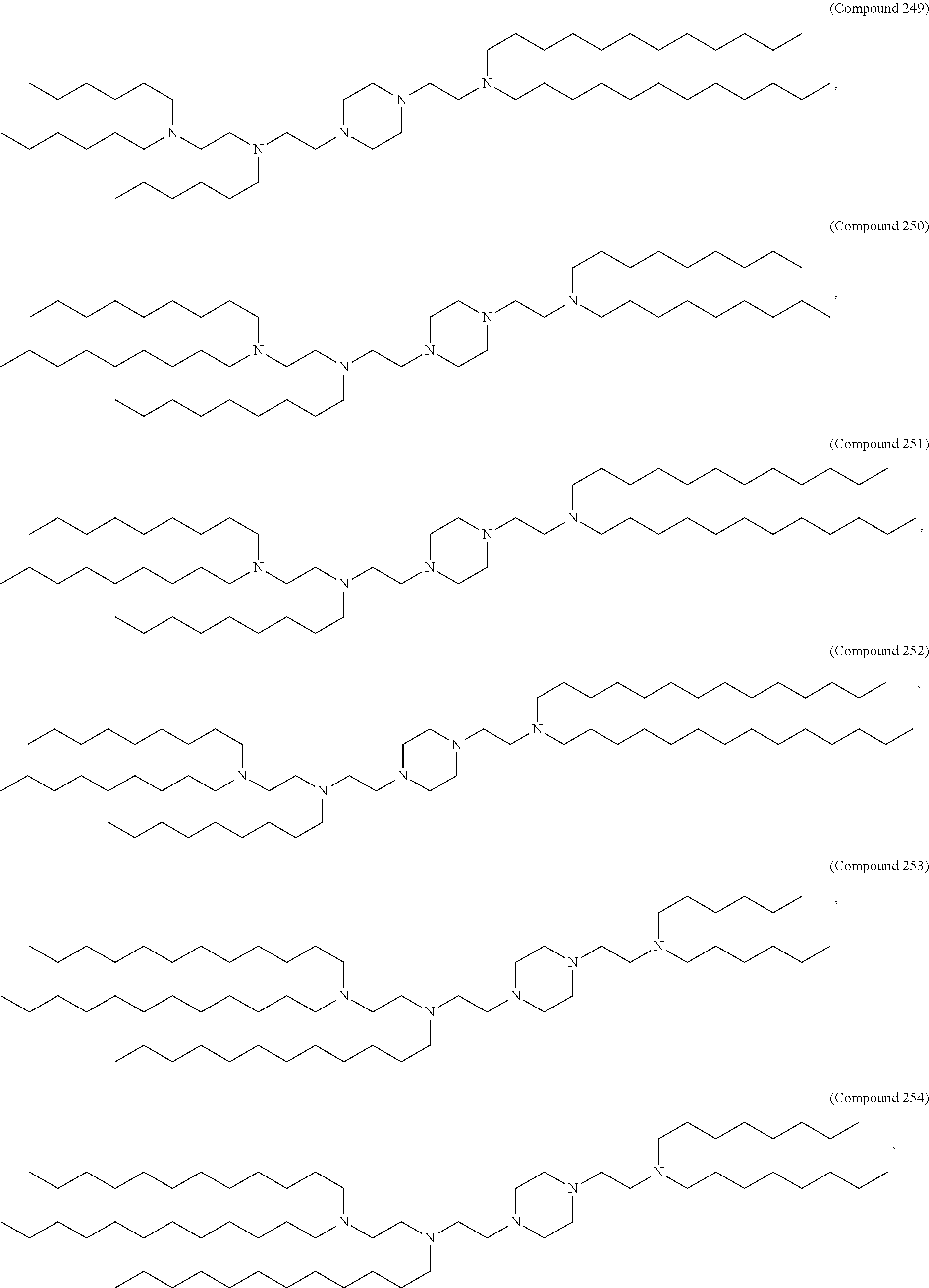

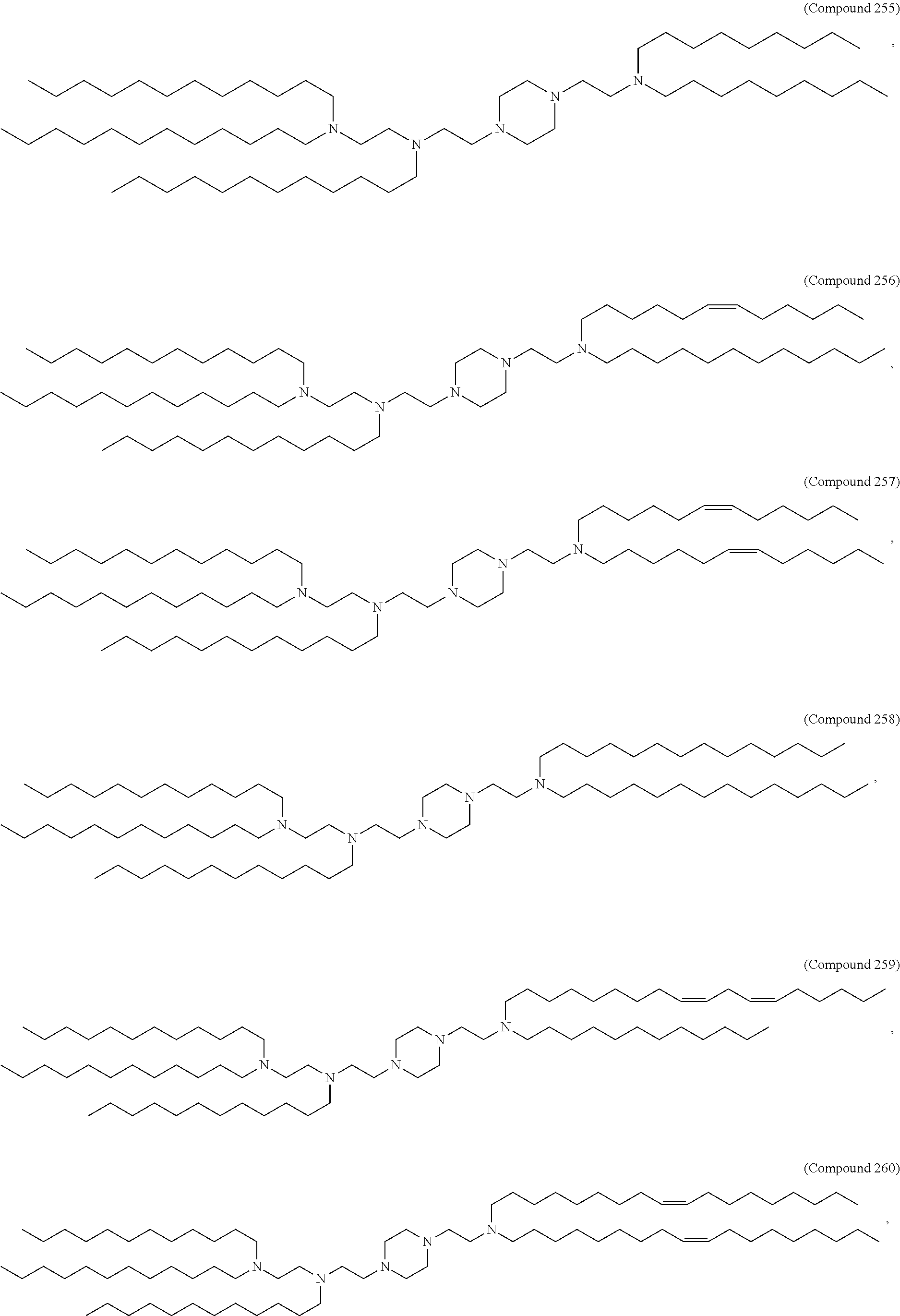

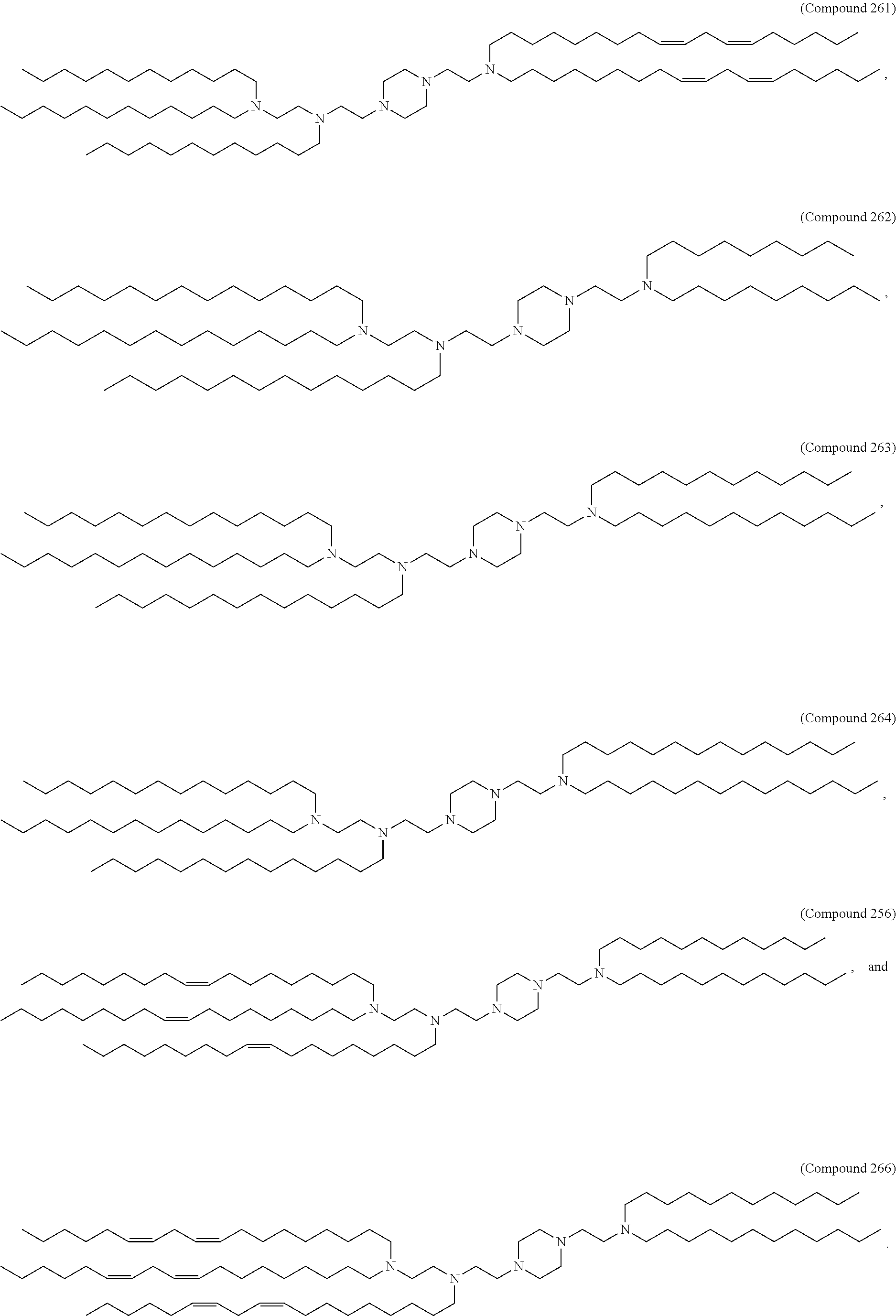

In other embodiments, the compound has the Formula (V)

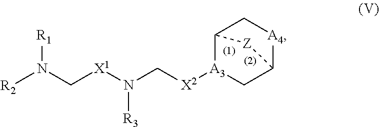

or a salt or isomer thereof, in which
A₃ is CH or N;
A₄ is CH₂ or NH; and at least one of A₃ and A₄ is N or NH;
Z is —CH₂— or absent wherein when Z is —CH₂—, the dashed lines (1) and (2) each represent a single bond; and when Z is absent, the dashed lines (1) and (2) are both absent;
R¹, R², and R³ are independently selected from the group consisting of C₅₋₂₀ alkyl, C₅₋₂₀ alkenyl, —R″MR′, —R*YR″, —YR″, and —R*OR″;
each M is independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹ and X² are independently selected from the group consisting of —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R¹ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl.

In some embodiments, the compound is of Formula (Va):

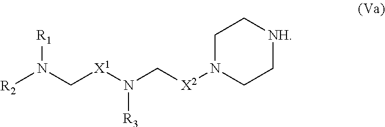

The compounds of Formula (V) or (Va) include one or more of the following features when applicable.

In some embodiments, Z is —CH₂—.

In some embodiments, Z is absent.

In some embodiments, at least one of A₃ and A₄ is N or NH.

In some embodiments, A₃ is N and A₄ is NH.

In some embodiments, A₃ is N and A₄ is CH₂.

In some embodiments, A₃ is CH and A₄ is NH.

In some embodiments, at least one of X¹ and X² is not —CH₂—. For example, in certain embodiments, X¹ is not —CH₂—. In some embodiments, at least one of X¹ and X² is —C(O)—.

In some embodiments, X² is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—.

In some embodiments, R₁, R₂, and R₃ are independently selected from the group consisting of C₅₋₂₀ alkyl and C₅₋₂₀ alkenyl. In some embodiments, R₁, R₂, and R₃ are the same. In certain embodiments, R₁, R₂, and R₃ are C₆, C₉, C₁₂, or C₁₄ alkyl. In other embodiments, R₁, R₂, and R₃ are C₁₈ alkenyl. For example, R₁, R₂, and R₃ may be linoleyl.

In some embodiments, the compound is selected from the group consisting of:

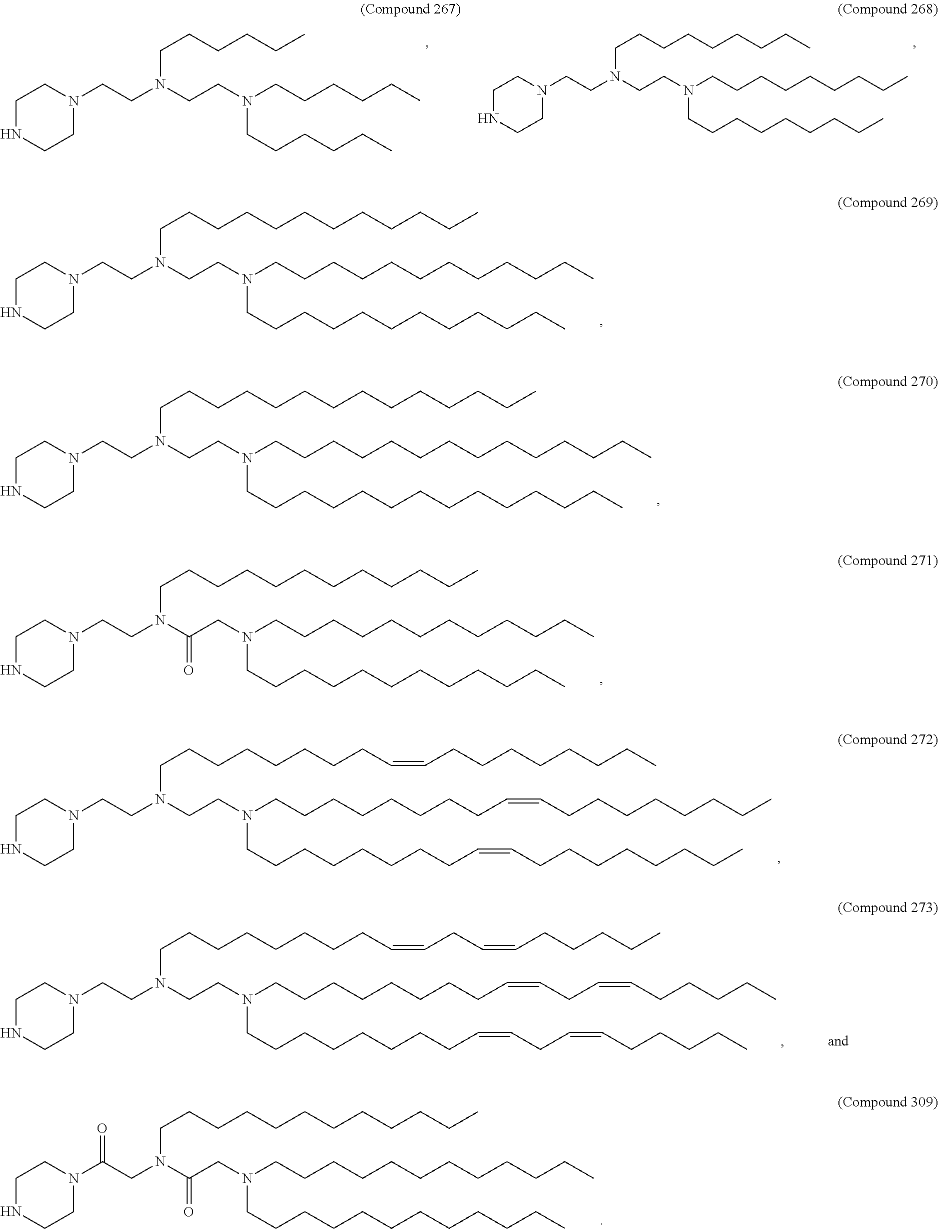

In another aspect, the disclosure provides a compound according to Formula (VI):

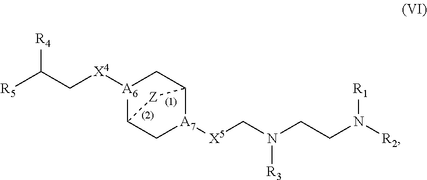

or a salt or isomer thereof, in which
A₆ and A₇ are each independently selected from CH or N, wherein at least one of A₆ and A₇ is N;
Z is —CH₂— or absent wherein when Z is —CH₂—, the dashed lines (1) and (2) each represent a single bond; and when Z is absent, the dashed lines (1) and (2) are both absent;
X⁴ and X⁵ are independently selected from the group consisting of —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
R₁, R₂, R₃, R₄, and R₅ each are independently selected from the group consisting of C₅₋₂₀ alkyl, C₅₋₂₀alkenyl, —R″MR′, —R*YR″, —YR″, and —R*OR″;
each M is independently selected from the group consisting of —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R¹ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl.

In some embodiments, R₁, R₂, R₃, R₄, and R₅ each are independently selected from the group consisting of C₆₋₂₀ alkyl and C₆₋₂₀ alkenyl.

In some embodiments, R₁ and R₂ are the same. In certain embodiments, R₁, R₂, and R₃ are the same.

In some embodiments, R₄ and R₅ are the same. In certain embodiments, R₁, R₂, R₃, R₄, and R₅ are the same.

In some embodiments, at least one of R₁, R₂, R₃, R₄, and R₅ is C₉₋₁₂ alkyl. In certain embodiments, each of R₁, R₂, R₃, R₄, and R₅ independently is C₉, C₁₂ or C₁₄ alkyl. In certain embodiments, each of R₁, R₂, R₃, R₄, and R₅ is C₉ alkyl.

In some embodiments, A₆ is N and A₇ is N. In some embodiments, A₆ is CH and A₇ is N.

In some embodiments, X₄ is —CH₂— and X₅ is —C(O)—. In some embodiments, X₄ and X₅ are —C(O)—.

In some embodiments, when A₆ is N and A₇ is N, at least one of X₄ and X₅ is not —CH₂—, e.g., at least one of X₄ and X₅ is —C(O)—. In some embodiments, when A₆ is N and A₇ is N, at least one of R₁, R₂, R₃, R₄, and R₅ is —R″MR′.

In some embodiments, at least one of R₁, R₂, R₃, R₄, and R₅ is not —R″MR′.

In some embodiments, the compound is

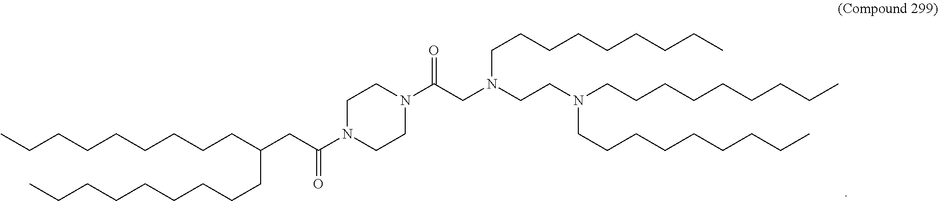 [SMILES: CCCCCCCCCC(CCCCCCCCC)CC(=O)N1CCN(C(=O)CN(CCCCCCCCC)CCN(CCCCCCCCC)CCCCCCCCC)CC1]

In an embodiment, the compound has the following formula:

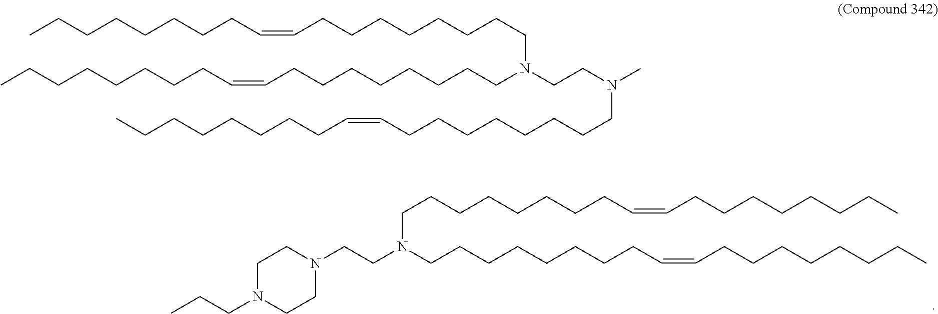

### PEG and PEG-Modified Lipids

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid. In some embodiments, a PEG lipid is DMG-PEG 2k or Compound 428.

In some embodiments, the PEG lipid is PEG-DMG. In some embodiments, the PEG lipid is PEG-DMG 2k. In some embodiments, a PEG lipid has the structure:

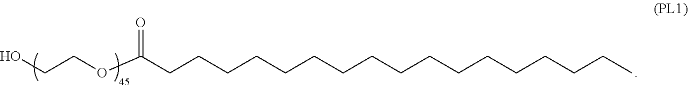

DMG-PEG 2k has the following structure:

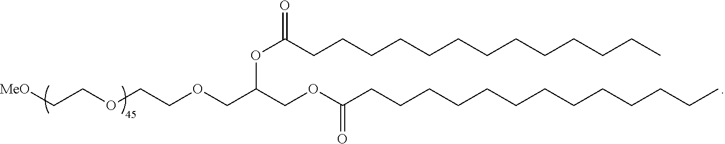

In some embodiments, the PEG-modified lipids are a modified form of PEG DMG. PEG-DMG has the following structure:

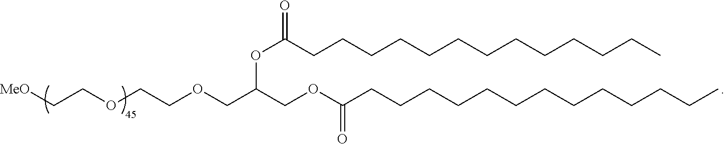

In some embodiments, the nanoparticle described herein comprises about 1 mol % to about 5 mol % of PEG-lipid. In some embodiments, the nanoparticle comprises about 1 mol % to about 2.5 mol % of PEG-lipid.

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (VII).

Provided herein are compounds of Formula (VII):

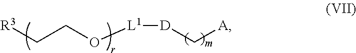

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
D is a moiety obtained by click chemistry or a moiety cleavable under physiological conditions;
m is 0, 1, 2, 3, 4, 5, 6, 7, 8, 9, or 10;
A is of the formula:

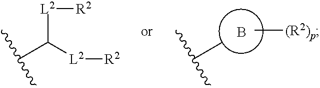

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁ 30 alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (VII) is a PEG-OH lipid (i.e., R³ is —OR^{O}, and R^{O} is hydrogen). In certain embodiments, the compound of Formula (VII) is of Formula (VII—OH):

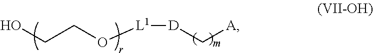

or a salt thereof.

In certain embodiments, D is a moiety obtained by click chemistry (e.g., triazole). In certain embodiments, the compound of Formula (VII) is of Formula (VII-a-1) or (VII-a-2):

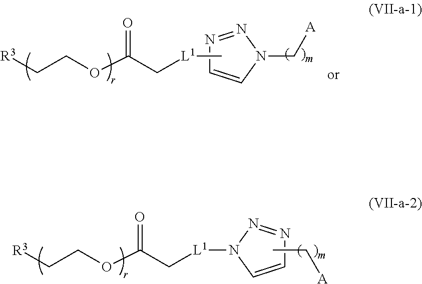

or a salt thereof.

In certain embodiments, the compound of Formula (VII) is of one of the following formulae:

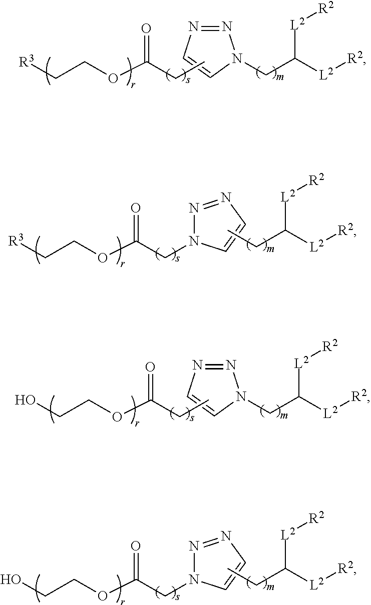

or a salt thereof, wherein
s is 0, 1, 2, 3, 4, 5, 6, 7, 8, 9, or 10.

In certain embodiments, the compound of Formula (VII) is of one of the following formulae:

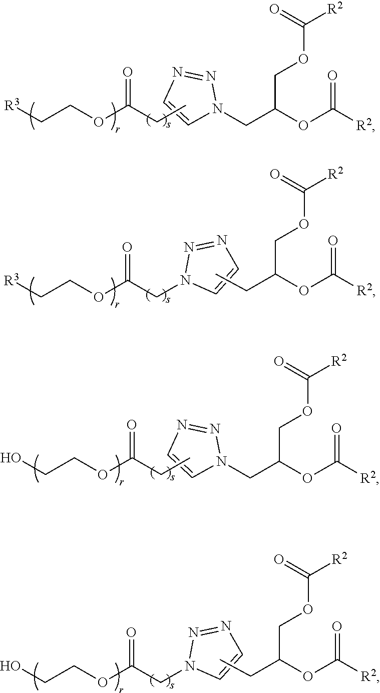

or a salt thereof.

In certain embodiments, a compound of Formula (VII) is of one of the following formulae:

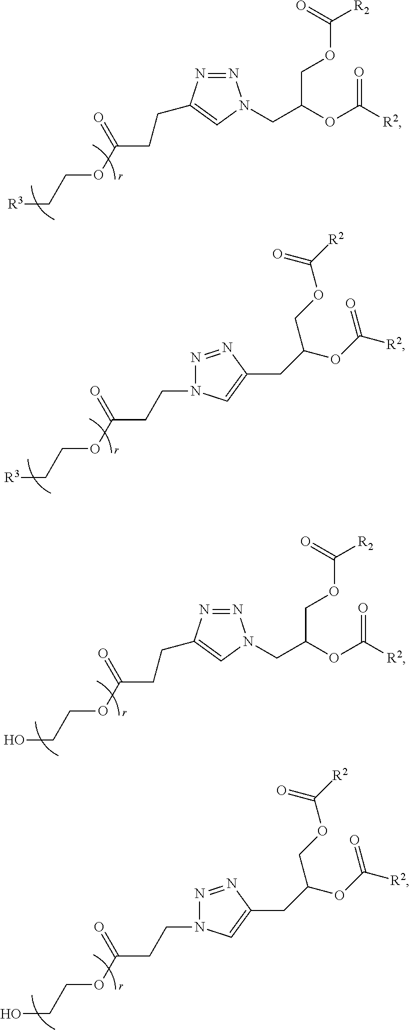

or a salt thereof.

In certain embodiments, a compound of Formula (VII) is of one of the following formulae, wherein r is 1-100:

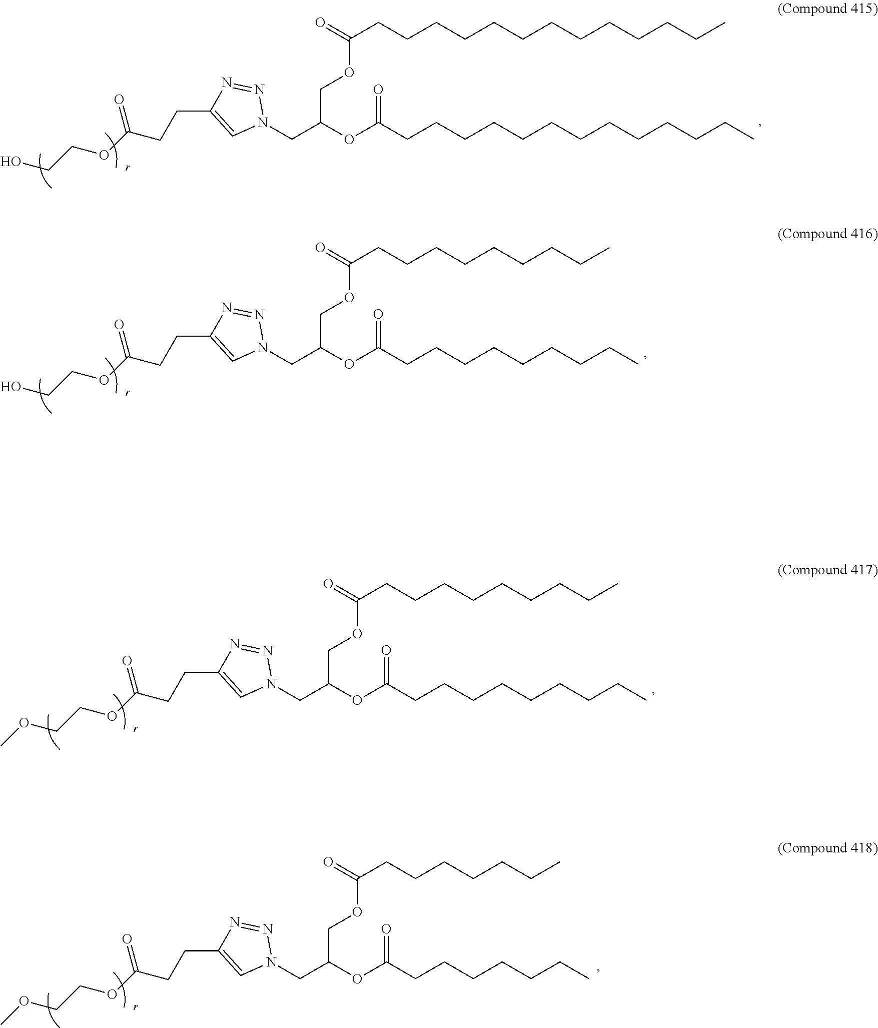

or a salt thereof.

In certain embodiments, D is a moiety cleavable under physiological conditions (e.g., ester, amide, carbonate, carbamate, urea). In certain embodiments, a compound of Formula (VII) is of Formula (VII-b-1) or (VII-b-2):

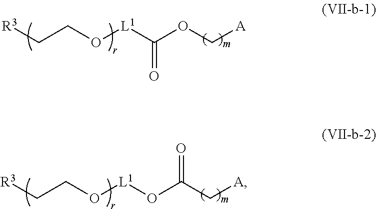

or a salt thereof.

In certain embodiments, a compound of Formula (VII) is of Formula (VII-b-1-OH) or (VII-b-2-OH):

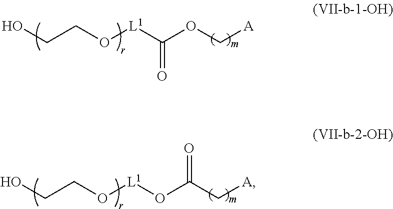

or a salt thereof.

In certain embodiments, the compound of Formula (VII) is of one of the following formulae:

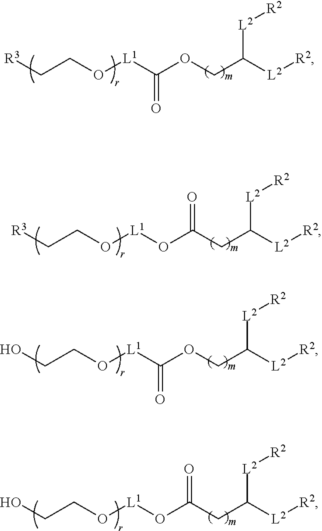

or a salt thereof.

In certain embodiments, a compound of Formula (VII) is of one of the following formulae:

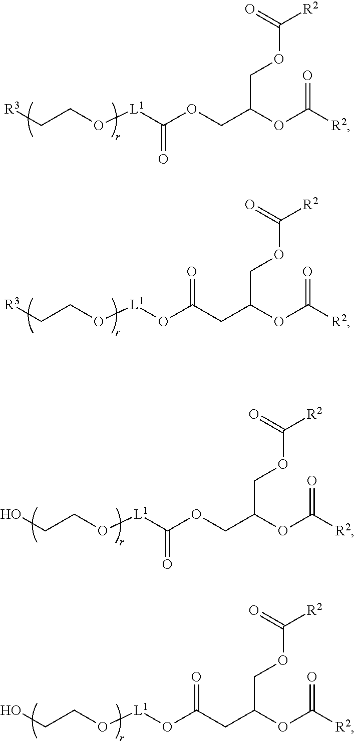

or a salt thereof.

In certain embodiments, a compound of Formula (VII) is of one of the following formulae:

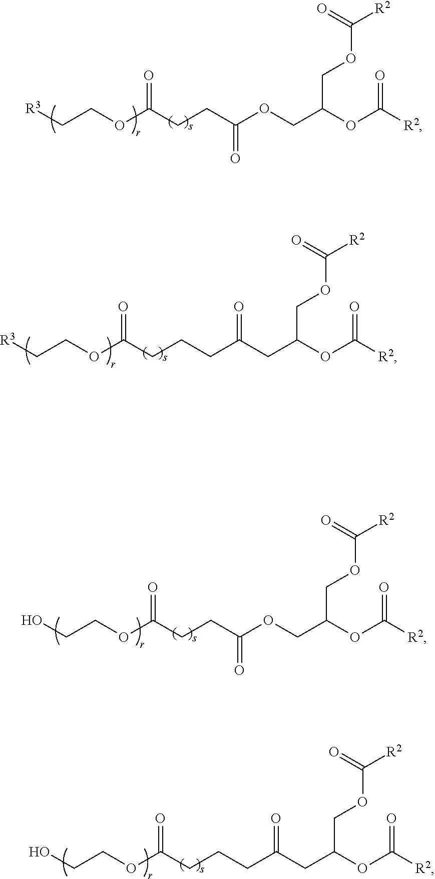

or a salt thereof.

In certain embodiments, a compound of Formula (VII) is of one of the following formulae:

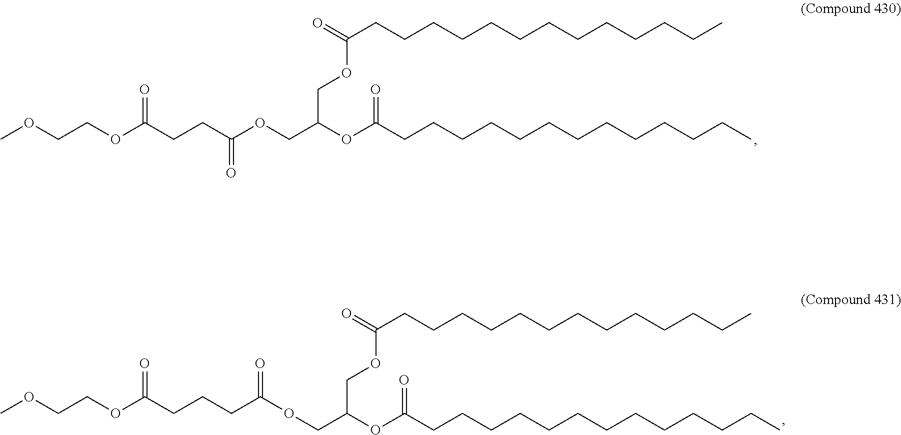

or salts thereof.

In certain embodiments, a PEG lipid useful in the present invention is a PEGylated fatty acid. In certain embodiments, a PEG lipid useful in the present invention is a compound of Formula (VIII). Provided herein are compounds of Formula (VIII):

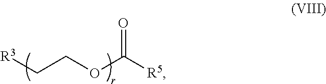

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and
each instance of R^{N} is independently hydrogen, optionally substituted alkyl, or a nitrogen protecting group.

In certain embodiments, the compound of Formula (VIII) is of Formula (VIII-OH):

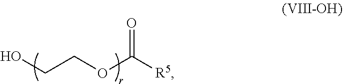

or a salt thereof.

In certain embodiments, a compound of Formula (VIII) is of one of the following formulae:

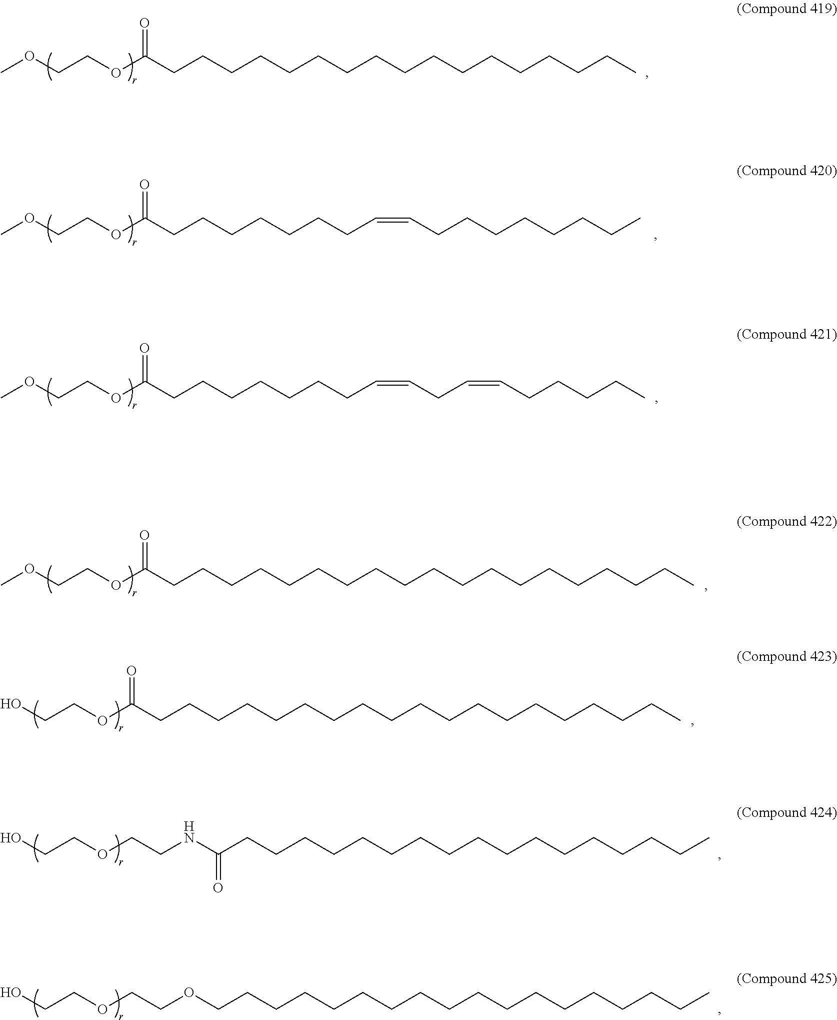

or a salt thereof. In some embodiments, r is 45.

In certain embodiments, a compound of Formula (VIII) is of one of the following formulae:

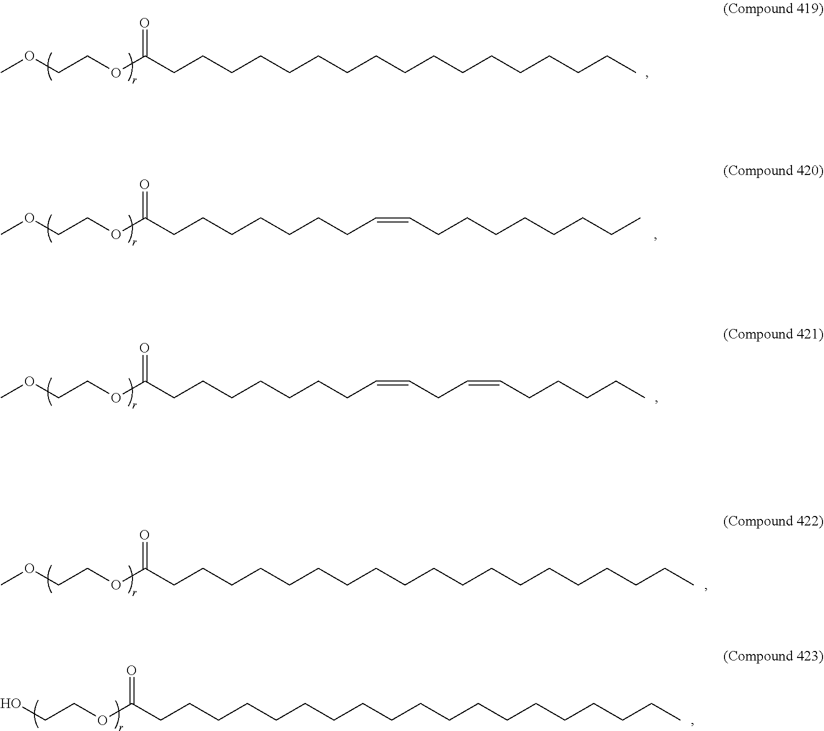

or a salt thereof. In some embodiments, r is 45.

In yet other embodiments the compound of Formula (VIII) is:

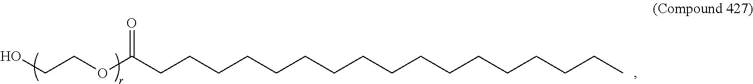

or a salt thereof.

In some embodiments, the compound of Formula (VIII) is

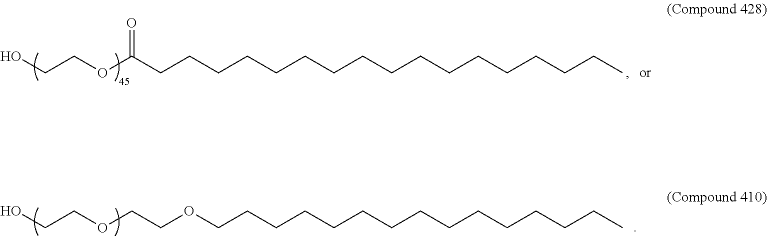

In certain embodiments, the PEG lipid is one of the following formula:

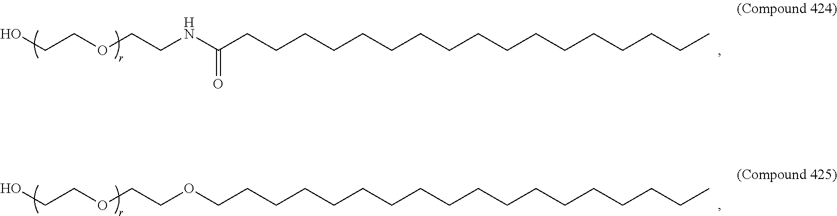

or a salt thereof. In some embodiments, r is 45.

In one embodiment, PEG-lipids useful in the present invention can be PEGylated lipids described in International Publication No. WO2012099755, the contents of which is herein incorporated by reference in its entirety.

Any of the PEG-lipids described herein may be modified to comprise one or more hydroxyl group on the PEG chain (OH-PEG-lipids) or one or more hydroxyl group on the lipid (PEG-lipid-OH). In some embodiments, the PEG-lipid is an OH-PEG-lipid. In some embodiments, the OH-PEG-lipid comprises a hydroxyl group at the terminus of the PEG chain. In some embodiments, the PEG-lipids described herein may be modified to comprise one or more alkyl group on the PEG chain (alkyl-PEG-lipid). In some embodiments, the alkyl-PEG-lipid is a methoxy-PEG-lipid.

In some embodiments, the LNP comprises about 0.1 mol % to about 5.0 mol %, about 0.5 mol % to about 5.0 mol %, about 1.0 mol % to about 5.0 mol %, about 1.0 mol % to about 2.5 mol %, about 0.5 mol % to about 2.0 mol %, or about 1.0 mol % to about 1.5 mol % of PEG-lipid. In some embodiments, the LNP comprises about 1.5 mol % or about 3.0 mol % PEG-lipid.

Certain of the LNPs provided herein comprise no or low levels of PEG-lipid. Some LNPs comprise less than 0.5 mol % PEG-lipid.

In some embodiments, PEG is used as a stabilizer. In some embodiments, the PEG stabilizer is a PEG-lipid. In some embodiments, the LNP comprises less than 0.5 mol % PEG stabilizer.

Other non-limiting examples of PEG lipids can be found in, e.g., International PCT Application Publication Nos. WO 2020/061284, published Mar. 26, 2020; and WO 2020/061295, published Mar. 26, 2020, the entire contents of each of which (including any generic or specific structures disclosed therein) is incorporated herein by reference.

### Phospholipids

Phospholipids, as defined herein, are any lipids that comprise a phosphate group. Phospholipids are a subset of non-cationic lipids. The lipid component of a lipid nanoparticle composition may include one or more phospholipids, such as one or more (poly)unsaturated lipids. Phospholipids may assemble into one or more lipid bilayers. In general, phospholipids may include a phospholipid moiety and one or more fatty acid moieties. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

In some embodiments, the nanoparticle described herein comprises about 5 mol % to about 15 mol % of phospholipid. In some embodiments, the nanoparticle comprises about 8 mol % to about 13 mol % of phospholipid. In some embodiments, the nanoparticle comprises about 10 mol % to about 12 mol % of phospholipid.

Phospholipids useful or potentially useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-diphytanoyl-sn-glycero-3-phosphocholine (4ME 16:0 PC), 1,2-diphytanoyl-sn-glycero-3-phospho-(1′-rac-glycerol) (sodium salt) (4ME 16:0 PG), 1,2-diphytanoyl-sn-glycero-3-phospho-L-serine (sodium salt) (4ME 16:0 PS), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, and 1,2-dioleoyl-sn-glycero-3-phospho-rac-(1-glycerol) sodium salt (DOPG), and sphingomyelin. Each possibility represents a separate embodiment of the present invention.

In some embodiments, a lipid nanoparticle composition includes DSPC. In certain embodiments, a lipid nanoparticle composition includes DOPE. In some embodiments, a lipid nanoparticle composition includes both DSPC and DOPE. In some embodiments, the lipid nanoparticle includes:

### 1,2-diphytanoyl-sn-glycero-3-phosphoethanolamine (4ME 16:0 PE)

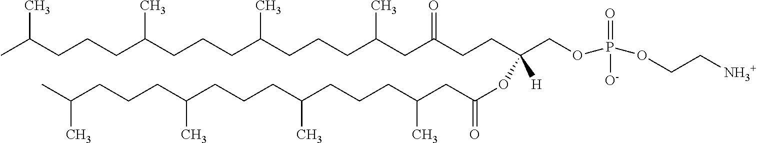 [SMILES: CC(C)CCCC(C)CCCC(C)CCCC(C)CC(=O)CC[C@H](COP(=O)([O-])OCC[NH3+])OC(=O)CC(C)CCCC(C)CCCC(C)CCCC(C)C]

### 1,2-diphytanoyl-sn-glycero-3-phosphocholine (4ME 16:0 PC)

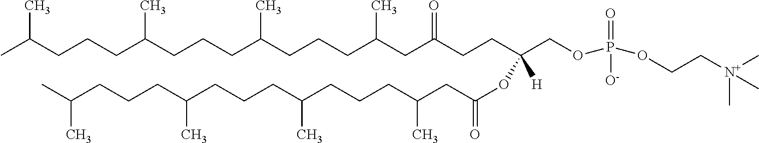 [SMILES: CC(C)CCCC(C)CCCC(C)CCCC(C)CC(=O)CC[C@H](COP(=O)([O-])OCC[N+](C)(C)C)OC(=O)CC(C)CCCC(C)CCCC(C)CCCC(C)C]

### 1,2-diphytanoyl-sn-glycero-3-phospho-(1′-rac-glycerol) (sodium salt) (4ME 16:0 PG), or

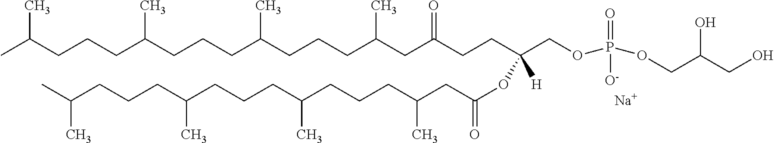 [SMILES: CC(C)CCCC(C)CCCC(C)CCCC(C)CC(=O)CC[C@H](COP(=O)([O-])OCC(O)CO)OC(=O)CC(C)CCCC(C)CCCC(C)CCCC(C)C ; [Na+]]

### 1,2-diphytanoyl-sn-glycero-3-phospho-L-serine (sodium salt) (4ME 16:0 PS)

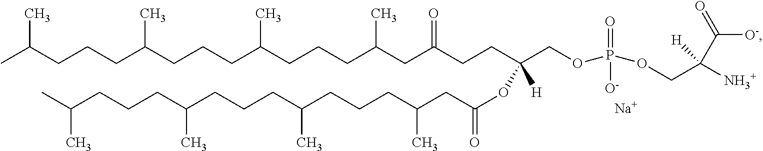

or a mixture thereof.

Examples of phospholipids include, but are not limited to, the following:

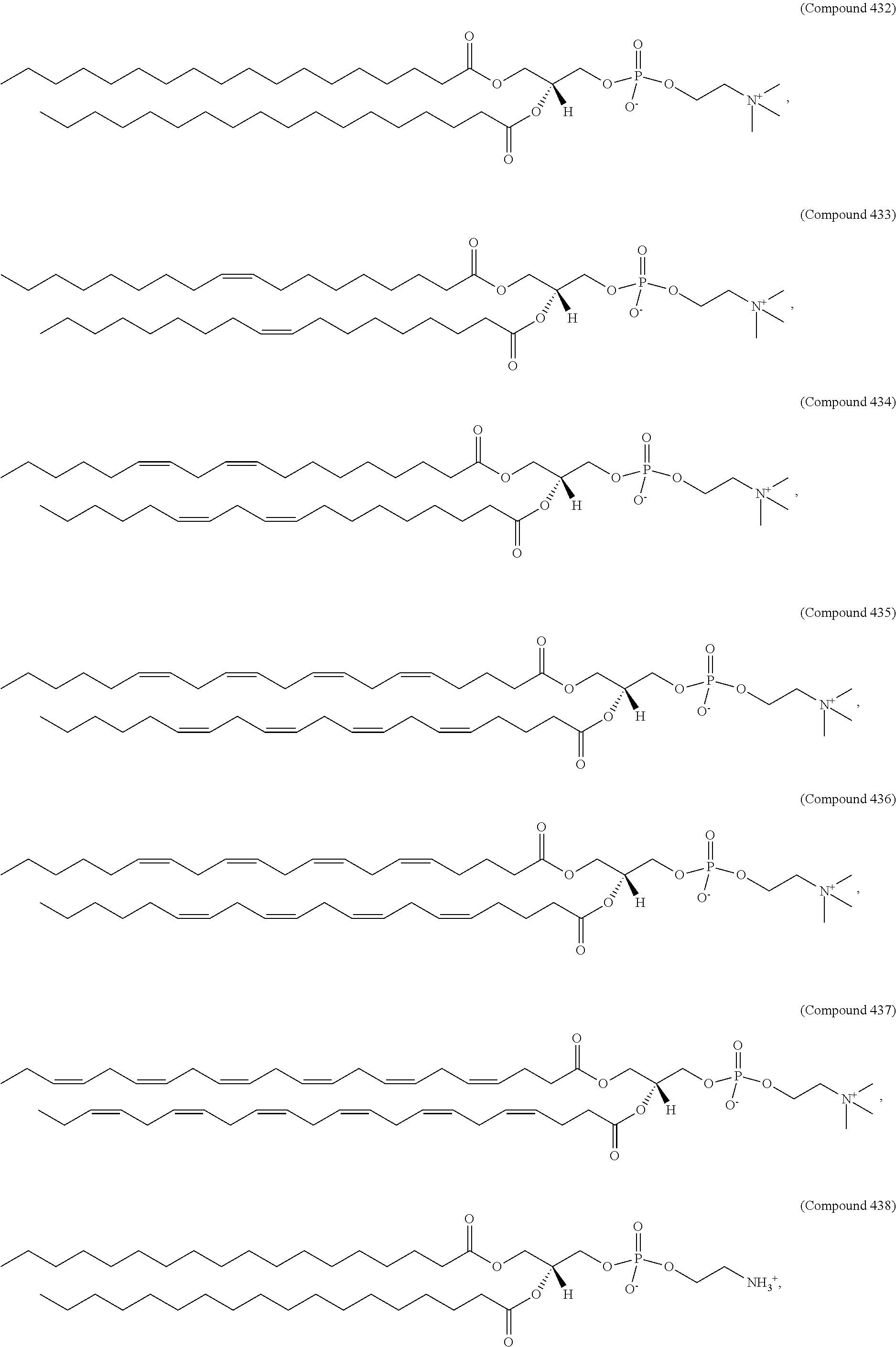

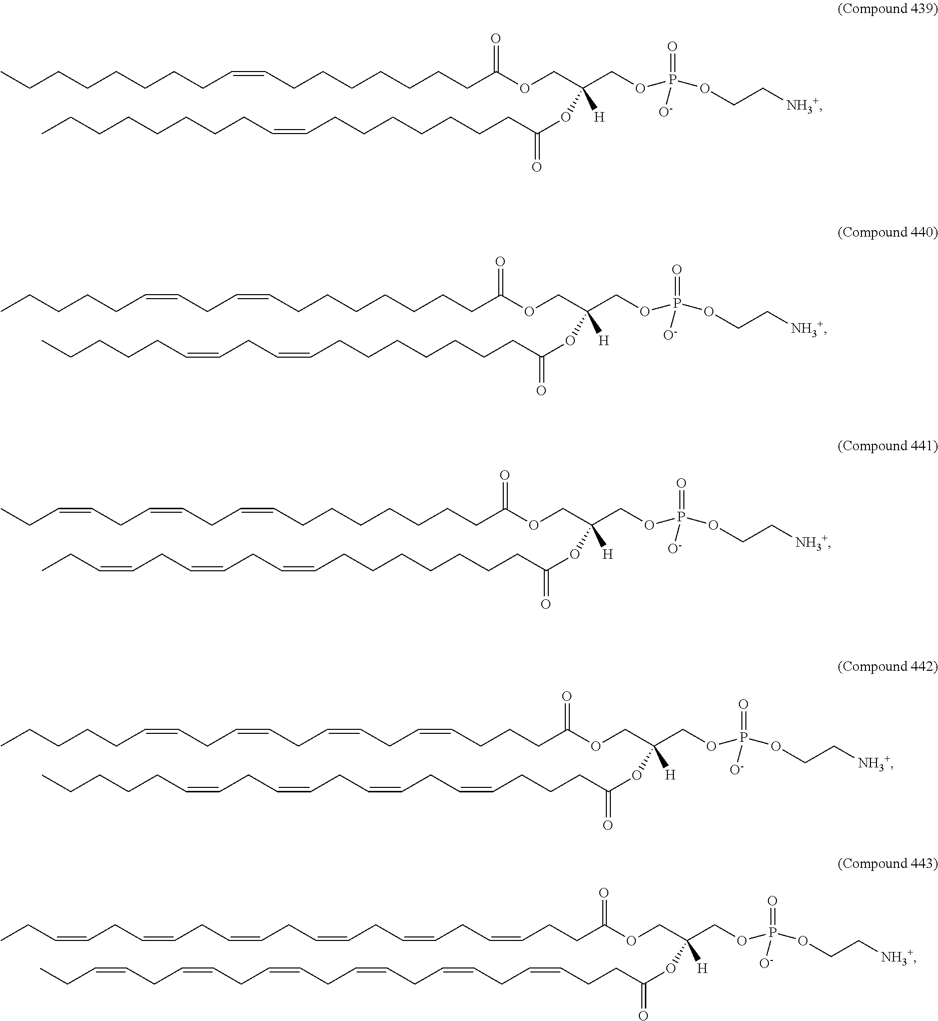

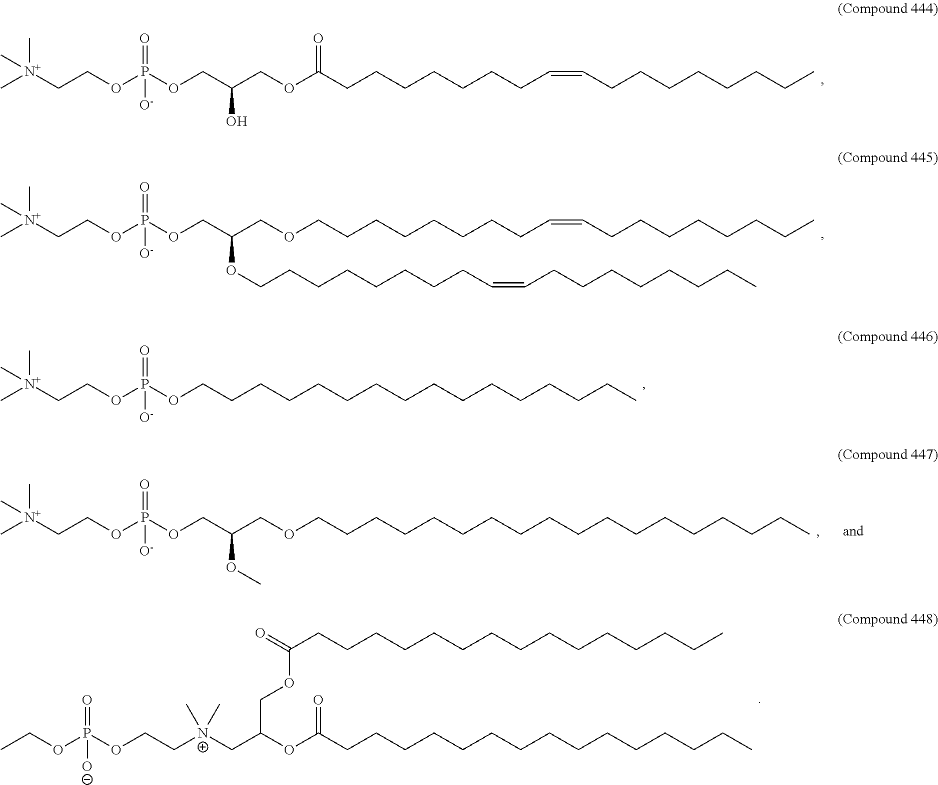

In certain embodiments, a phospholipid useful or potentially useful in the present invention is an analog or variant of DSPC.

In certain embodiments, a phospholipid useful or potentially useful in the present invention is a compound of Formula (IX):

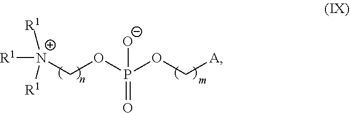

or a salt thereof, wherein:
each R¹ is independently H or optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

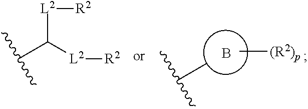

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

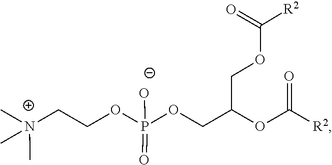

wherein each instance of R² is independently unsubstituted alkyl, unsubstituted alkenyl, or unsubstituted alkynyl.

In certain embodiments, a phospholipid useful or potentially useful in the present invention is a compound of Formula (IX):

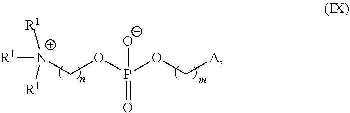

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

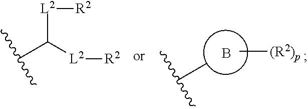

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁ 30 alkyl, optionally substituted C₁₋₃₀alkenyl, or optionally substituted C₁ 30 alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

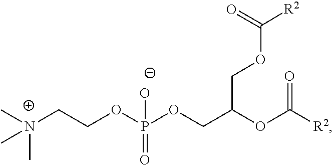

wherein each instance of R² is independently unsubstituted alkyl, unsubstituted alkenyl, or unsubstituted alkynyl.

In some embodiments, the phospholipid is selected from: 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-diphytanoyl-sn-glycero-3-phosphocholine (4ME 16:0 PC), 1,2-diphytanoyl-sn-glycero-3-phospho-(1′-rac-glycerol) (sodium salt) (4ME 16:0 PG), 1,2-diphytanoyl-sn-glycero-3-phospho-L-serine (sodium salt) (4ME 16:0 PS), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and Sphingomyelin.

In some embodiments, the phospholipid is DSPC, DOPE, or combinations thereof. In some embodiments, the phospholipid is DSPC. In some embodiments, the phospholipid is DOPE. In some embodiments, the phospholipid is 4ME 16:0 PE, 4ME 16:0 PC, 4ME 16:0 PG, 4ME 16:0 PS, or combination thereof.

In some embodiments, the phospholipid is N-lauroyl-D-erythro-sphinganylphosphorylcholine.

### Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

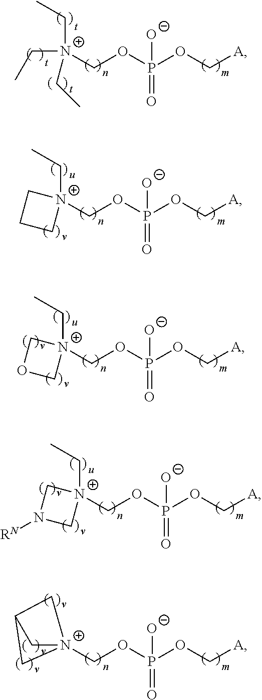

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, the compound of Formula (IX) is of one of the following formulae:

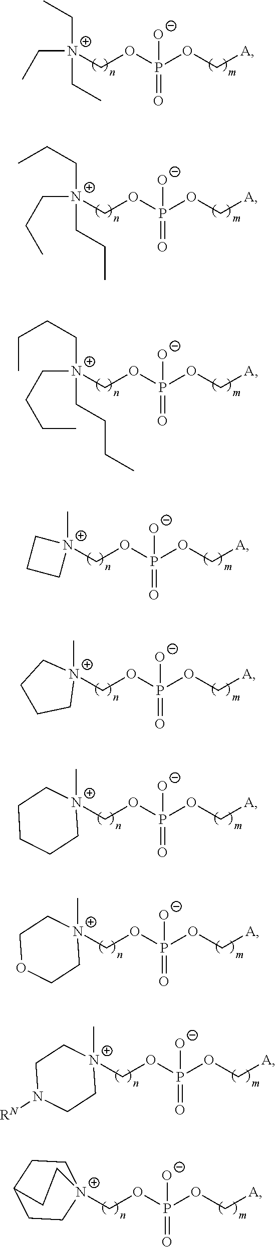

or a salt thereof.

In certain embodiments, a compound of Formula (IX) is one of the following:

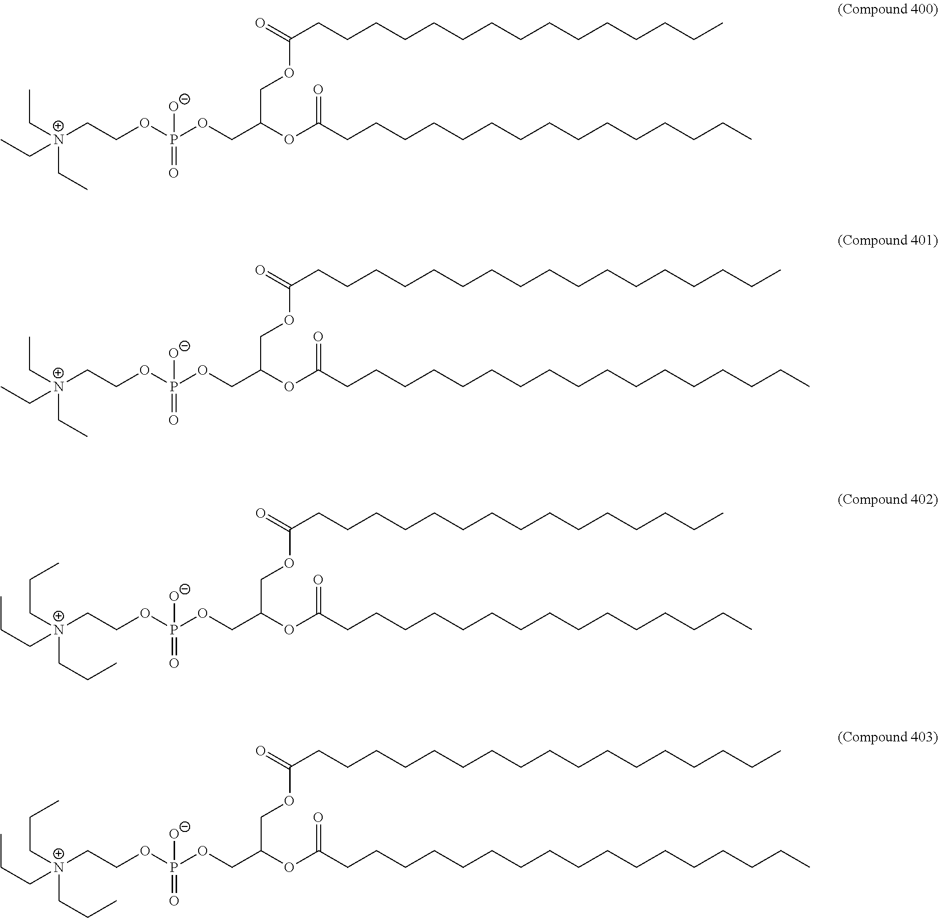

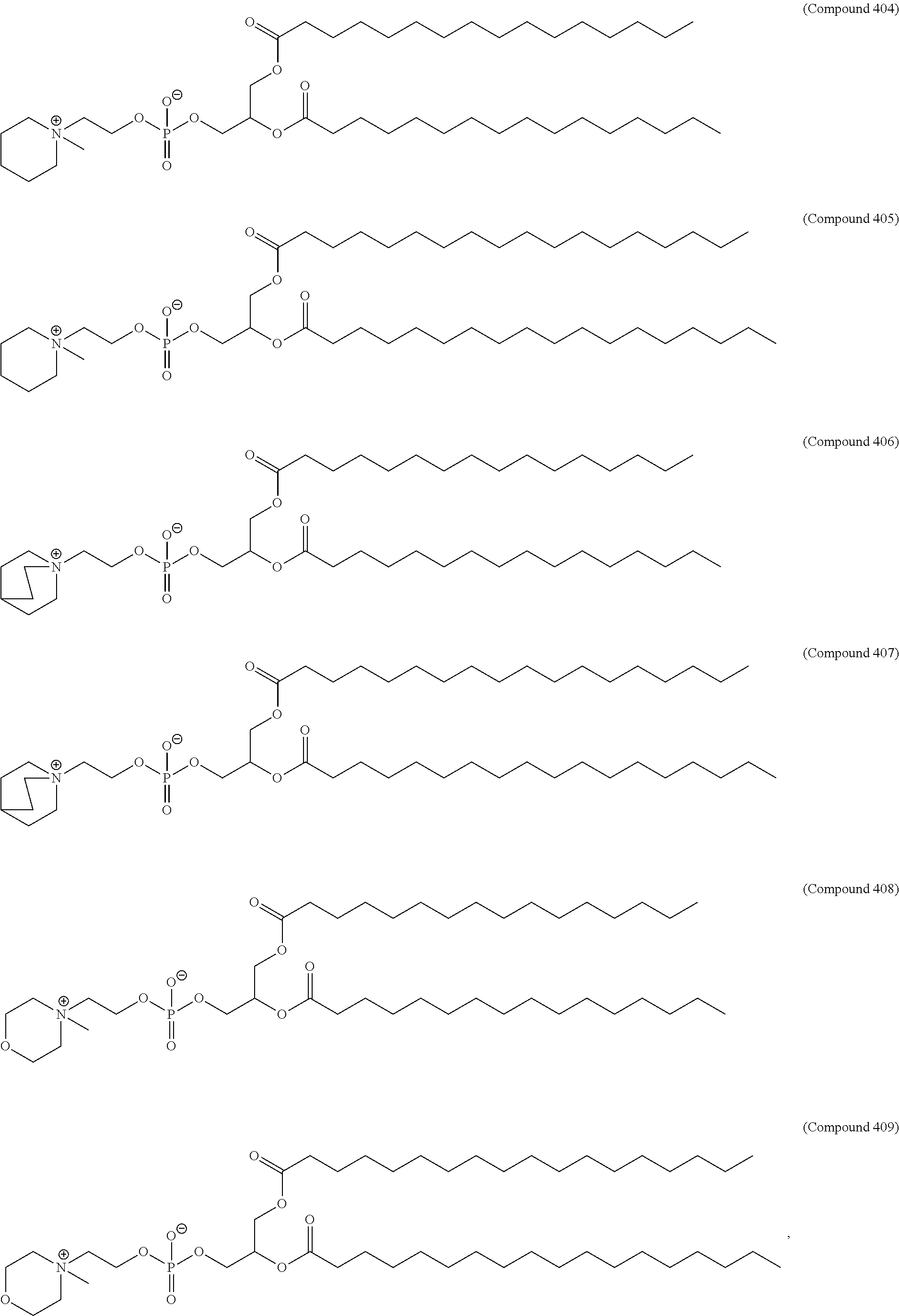 [SMILES: CCCCCCCCCCCCCCCC(=O)OCC(COP(=O)([O-])OCC[N+]1(C)CCCCC1)OC(=O)CCCCCCCCCCCCCCC ; CCCCCCCCCCCCCCCCCC(=O)OCC(COP(=O)([O-])OCC[N+]1(C)CCCCC1)OC(=O)CCCCCCCCCCCCCCCCC ; CCCCCCCCCCCCCCCC(=O)OCC(COP(=O)([O-])OCC[N+]12CCC(CC1)CC2)OC(=O)CCCCCCCCCCCCCCC ; CCCCCCCCCCCCCCCCCC(=O)OCC(COP(=O)([O-])OCC[N+]12CCC(CC1)CC2)OC(=O)CCCCCCCCCCCCCCCCC ; CCCCCCCCCCCCCCCC(=O)OCC(COP(=O)([O-])OCC[N+]1(C)CCOCC1)OC(=O)CCCCCCCCCCCCCCC ; CCCCCCCCCCCCCCCCCC(=O)OCC(COP(=O)([O-])OCC[N+]1(C)CCOCC1)OC(=O)CCCCCCCCCCCCCCCCC]

or a salt thereof.

In certain embodiments, a compound of Formula (IX) is of Formula (IX-a):

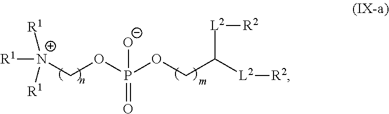

or a salt thereof.

In certain embodiments, phospholipids useful or potentially useful in the present invention comprise a modified core. In certain embodiments, a phospholipid with a modified core described herein is DSPC, or analog thereof, with a modified core structure. For example, in certain embodiments of Formula (IX-a), group A is not of the following formula:

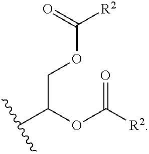

In certain embodiments, the compound of Formula (IX-a) is of one of the following formulae:

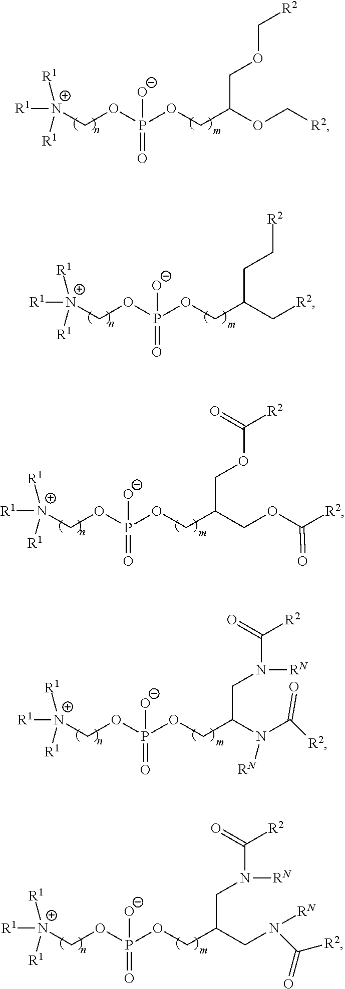

or a salt thereof.

In certain embodiments, a compound of Formula (IX) is one of the following:

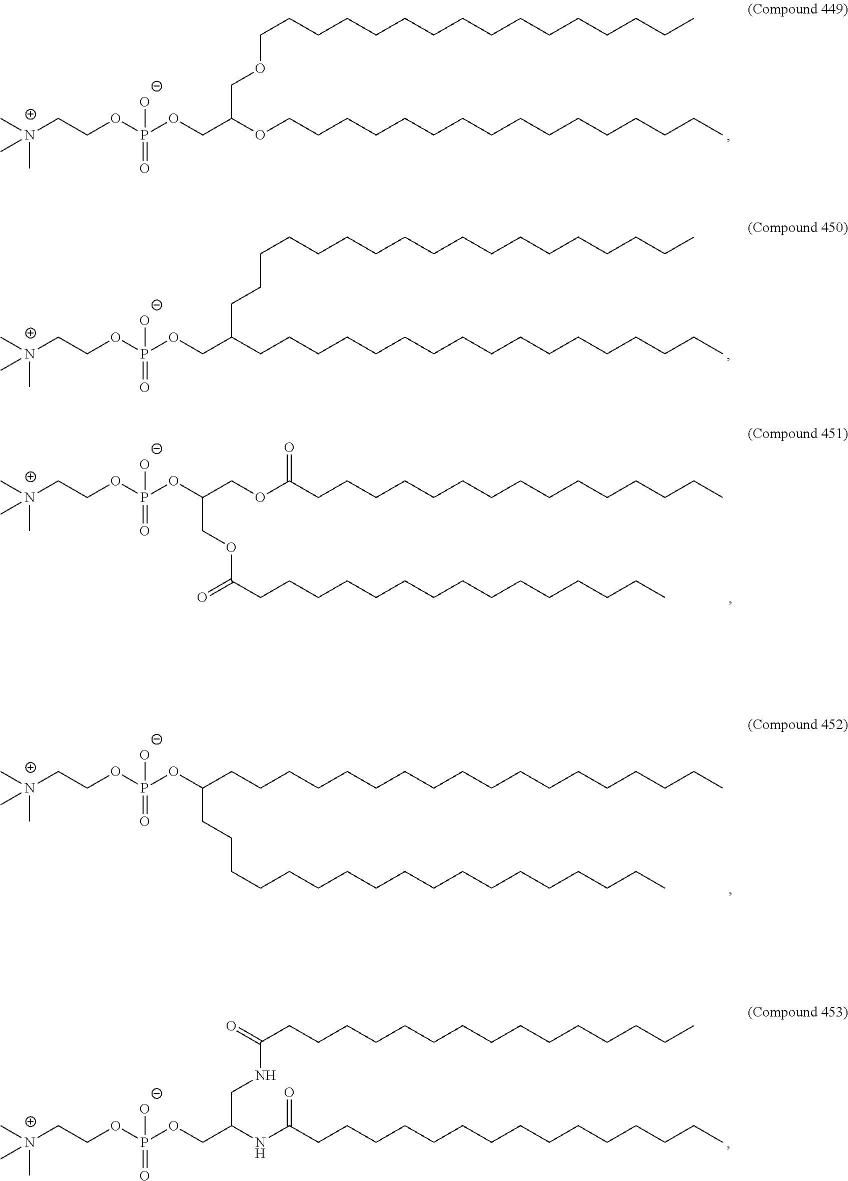

or salts thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IX) is of Formula (IX-b):

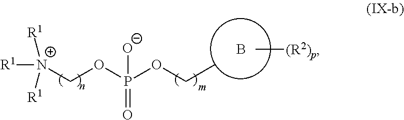

or a salt thereof.

In certain embodiments, the compound of Formula (IX-b) is of Formula (IX-b-1):

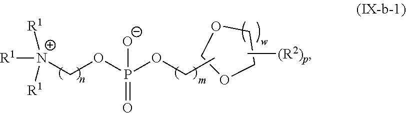

or a salt thereof, wherein:
w is 0, 1, 2, or 3.

In certain embodiments, the compound of Formula (TX-b) is of Formula (IX-b-2):

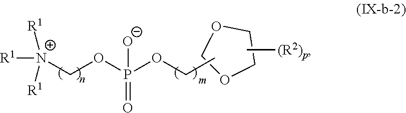

or a salt thereof.

In certain embodiments, the compound of Formula (IX-b) is of Formula (IX-b-3):

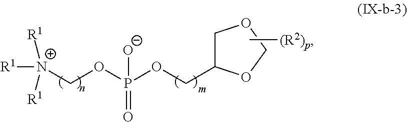

or a salt thereof.

In certain embodiments, the compound of Formula (IX-b) is of Formula (IX-b-4):

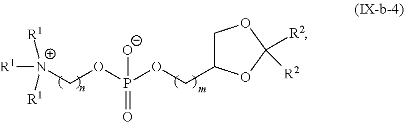

or a salt thereof.

In certain embodiments, the compound of Formula (IX-b) is one of the following:

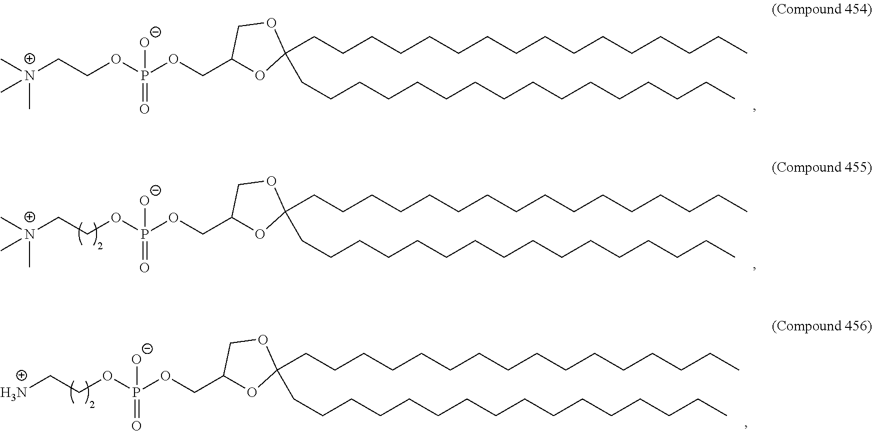

or salts thereof.

### Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IX) is of Formula (IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (IX) is of Formula (IX-c):

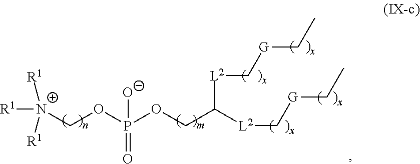

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (IX-c) is of Formula (IX-c-1):

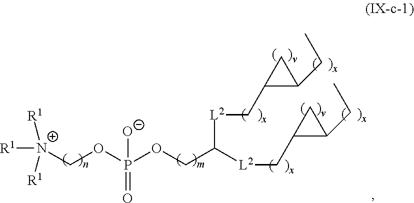

or salt thereof, wherein:
each instance of v is independently 1, 2, or 3.

In certain embodiments, the compound of Formula (IX-c) is of Formula (IX-c-2):

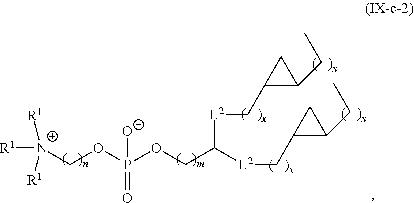

or a salt thereof.

In certain embodiments, the compound of Formula (IX-c) is of the following formula:

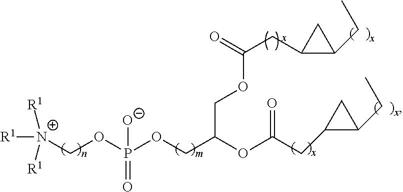

or a salt thereof.

In certain embodiments, the compound of Formula (IX-c) is the following:

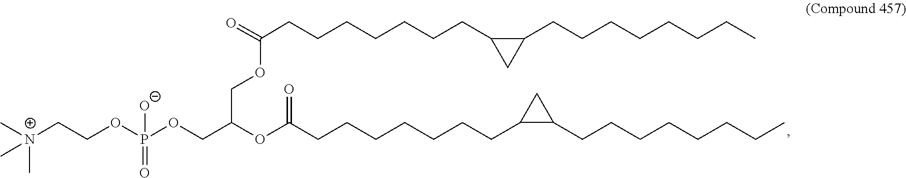 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (IX-c) is of Formula (IX-c-3):

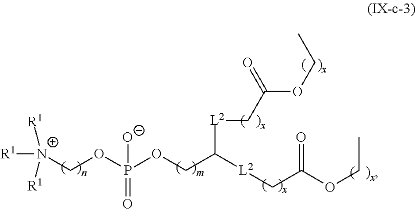

or a salt thereof.

In certain embodiments, the compound of Formula (IX-c) is of the following formulae:

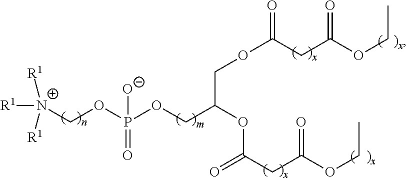

or a salt thereof.

In certain embodiments, the compound of Formula (IX-c) is the following:

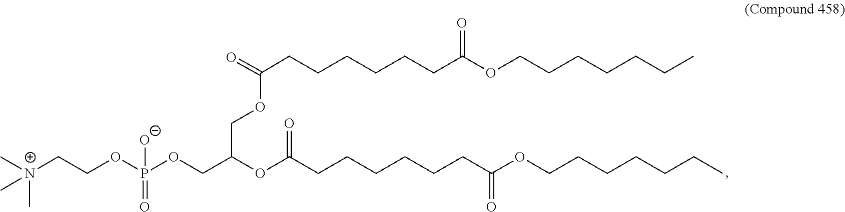 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IX), wherein n is 1, 3, 4, 5, 6, 7, 8, 9, or 10. For example, in certain embodiments, a compound of Formula (IX) is of one of the following formulae:

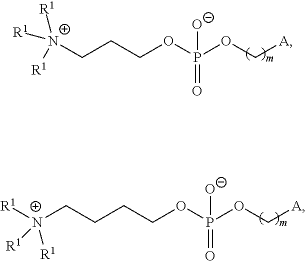

or a salt thereof.

In certain embodiments, a compound of Formula (IX) is one of the following:

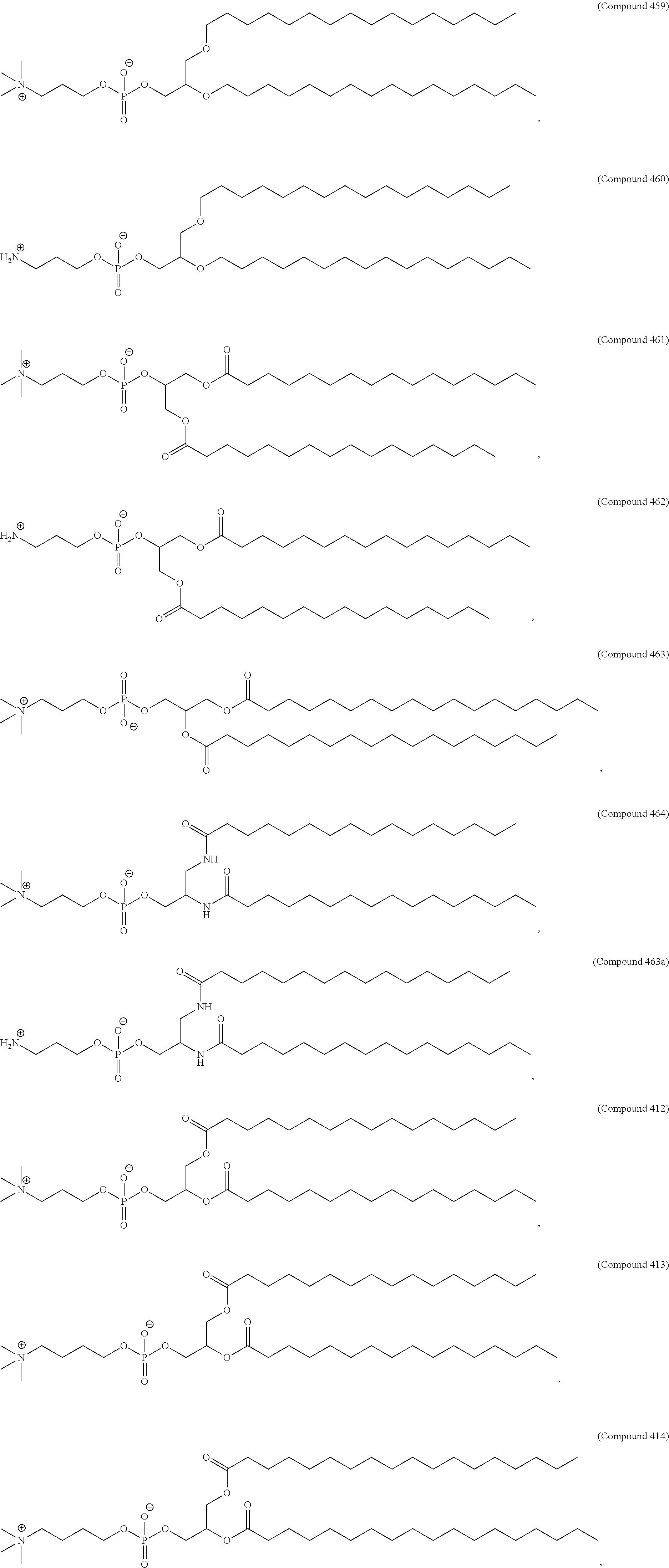

or salts thereof.

### Alternative Lipids

In certain embodiments, an alternative lipid is used in place of a phospholipid of the invention. Non-limiting examples of such alternative lipids include the following:

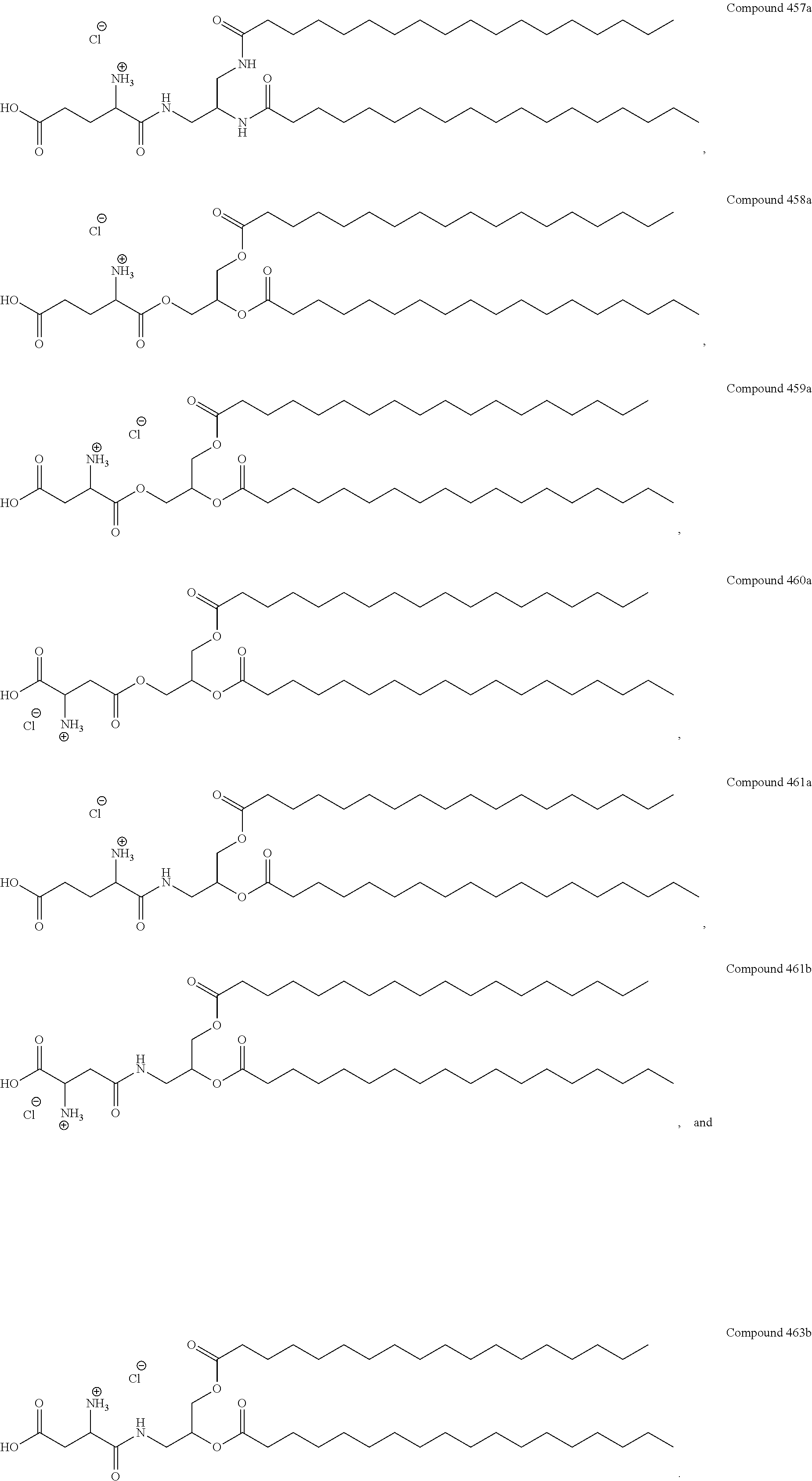

### Structural Lipids

The lipid component of a lipid nanoparticle composition may include one or more structural lipids. Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. In some embodiments, the structural lipid is β-sitosterol. In certain embodiments, the structural lipid is cholesteryl hemisuccinate. Cholesteryl hemisuccinate has the following structure:

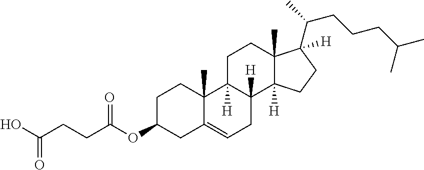

Examples of structural lipids include, but are not limited to, the following:

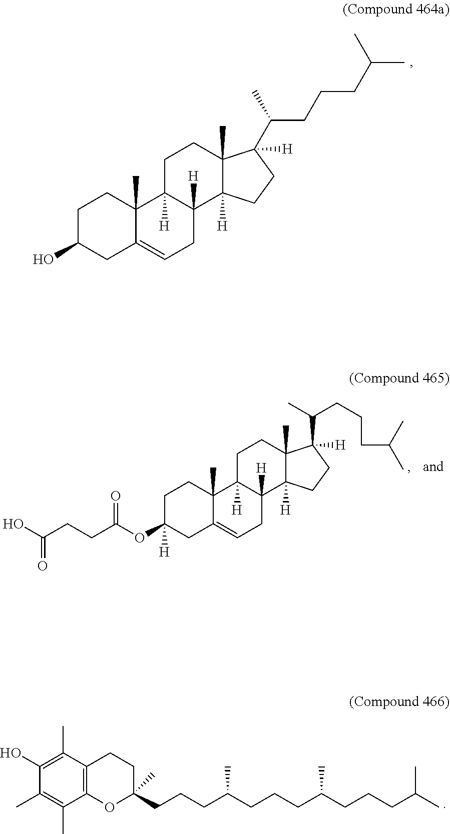

In some embodiments, the nanoparticle described herein can comprise about 20 mol % to about 60 mol % structural lipid. In some embodiments, the nanoparticle comprises about 30 mol % to about 50 mol % of structural lipid. In some embodiments, the nanoparticle comprises about 35 mol % of structural lipid. In some embodiments, the nanoparticle comprises about 40 mol % structural lipid. In some embodiments, the structural lipid is cholesterol or a compound having the following structure:

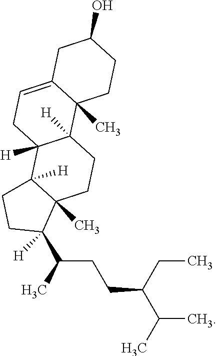

### Molar Ratios of Lipid Nanoparticle Components

In some embodiments, the polynucleotide (e.g., polynucleotide encoding an antigen) is formulated with a delivery agent comprising, e.g., a compound having the Formula (I), e.g., any of Compounds 1-232, e.g., Compound 18; a compound having the Formula (III), (IV), (V), or (VI), e.g., any of Compounds 233-342, e.g., Compound 236; a compound having the Formula (VIII), e.g., any of Compounds 419-428, e.g., Compound 428, or a compound having the Formula A1, A2, A3, A4, or A5, e.g., any one of SA1-SA41, or any combination thereof. In some embodiments, the delivery agent comprises Compound 18, DSPC, Cholesterol, Compound 428 or PEG-DMG, and SA3 or a salt thereof, e.g., with a mole ratio of about 47.6±25:9.5±8:36.6±20:1.4±1.25:4.9±2.5. In some embodiments, the delivery agent comprises Compound 18, DSPC, Cholesterol, Compound 428 or PEG-DMG, and SA3 or a salt thereof, e.g., with a mole ratio of about 47.6±12.5:9.5±4:36.6±10:1.4±0.75:4.9±1.25. In some embodiments, the delivery agent comprises Compound 18, DSPC, Cholesterol, and Compound 428 or PEG-DMG, e.g., with a mole ratio of about 47.6±6.25:9.5±2:36.6±5:1.4±0.375:4.9±0.625. In some embodiments, the delivery agent comprises Compound 18, DSPC, Cholesterol, Compound 428 or PEG-DMG, and SA3 or a salt thereof, e.g., with a mole ratio of about 47.6:9.5:36.6:1.4:4.9. In some embodiments, the delivery agent comprises Compound 236, DSPC, Cholesterol, Compound 428 or PEG-DMG, and SA3 or a salt thereof, e.g., with a mole ratio of about 47.6±25:9.5±8:36.6±20:1.4±1.25:4.9±2.5. In some embodiments, the delivery agent comprises Compound 236, DSPC, Cholesterol, Compound 428 or PEG-DMG, and SA3 or a salt thereof, e.g., with a mole ratio of about 47.6±12.5:9.5±4:36.6±10:1.4±0.75:4.9±1.25. In some embodiments, the delivery agent comprises Compound 236, DSPC, Cholesterol, and Compound 428 or PEG-DMG, e.g., with a mole ratio of about 47.6±6.25:9.5±2:36.6±5:1.4±0.375:4.9±0.625. In some embodiments, the delivery agent comprises Compound 236, DSPC, Cholesterol, Compound 428 or PEG-DMG, and SA3 or a salt thereof, e.g., with a mole ratio of about 47.6:9.5:36.6:1.4:4.9. In some embodiments, the delivery agent comprises Compound 18, DSPC, Cholesterol, Compound 428 or PEG-DMG, and SA3 or a salt thereof, e.g., with a mole ratio of about 47.3±25:9.5±8:36.4±20:1.4±1.25:5.5±2.5. In some embodiments, the delivery agent comprises Compound 18, DSPC, Cholesterol, Compound 428 or PEG-DMG, and SA3 or a salt thereof, e.g., with a mole ratio of about 47.3±12.5:9.5±4:36.4±10:1.4±0.75:5.5±1.25. In some embodiments, the delivery agent comprises Compound 18, DSPC, Cholesterol, and Compound 428 or PEG-DMG, e.g., with a mole ratio of about 47.3±6.25:9.5±2:36.4±5:1.4±0.375:5.5±0.625. In some embodiments, the delivery agent comprises Compound 18, DSPC, Cholesterol, Compound 428 or PEG-DMG, and SA3 or a salt thereof, e.g., with a mole ratio of about 47.3:9.5:36.4:1.4:5.5. In some embodiments, the delivery agent comprises Compound 236, DSPC, Cholesterol, Compound 428 or PEG-DMG, and SA3 or a salt thereof, e.g., with a mole ratio of about 47.3±25:9.5±8:36.4±20:1.4±1.25:5.5±2.5. In some embodiments, the delivery agent comprises Compound 236, DSPC, Cholesterol, Compound 428 or PEG-DMG, and SA3 or a salt thereof, e.g., with a mole ratio of about 47.3±12.5:9.5±4:36.4±10:1.4±0.75:5.5±1.25. In some embodiments, the delivery agent comprises Compound 236, DSPC, Cholesterol, and Compound 428 or PEG-DMG, e.g., with a mole ratio of about 47.3±6.25:9.5±2:36.4±5:1.4±0.375:5.5±0.625. In some embodiments, the delivery agent comprises Compound 236, DSPC, Cholesterol, Compound 428 or PEG-DMG, and SA3 or a salt thereof, e.g., with a mole ratio of about 47.3:9.5:36.4:1.4:5.5. In some embodiments, the delivery agent comprises Compound 18, DSPC, Cholesterol, Compound 428 or PEG-DMG, and SA3 or a salt thereof, e.g., with a mole ratio of about 45.8±25:10.5±8:36.8±20:1.4±1.25:5.5±2.5. In some embodiments, the delivery agent comprises Compound 18, DSPC, Cholesterol, Compound 428 or PEG-DMG, and SA3 or a salt thereof, e.g., with a mole ratio of about 45.8±12.5:10.5±4:36.8±10:1.4±0.75:5.5±1.25. In some embodiments, the delivery agent comprises Compound 18, DSPC, Cholesterol, and Compound 428 or PEG-DMG, e.g., with a mole ratio of about 45.8±6.25:10.5±2:36.8±5:1.4±0.375:5.5±0.625. In some embodiments, the delivery agent comprises Compound 18, DSPC, Cholesterol, Compound 428 or PEG-DMG, and SA3 or a salt thereof, e.g., with a mole ratio of about 45.8:10.5:36.8:1.4:5.5. In some embodiments, the delivery agent comprises Compound 236, DSPC, Cholesterol, Compound 428 or PEG-DMG, and SA3 or a salt thereof, e.g., with a mole ratio of about 45.8±25:10.5±8:36.8±20:1.4±1.25:5.5±2.5. In some embodiments, the delivery agent comprises Compound 236, DSPC, Cholesterol, Compound 428 or PEG-DMG, and SA3 or a salt thereof, e.g., with a mole ratio of about 45.8±12.5:10.5±4:36.8±10:1.4±0.75:5.5±1.25. In some embodiments, the delivery agent comprises Compound 236, DSPC, Cholesterol, and Compound 428 or PEG-DMG, e.g., with a mole ratio of about 45.8±6.25:10.5±2:36.8±5:1.4±0.375:5.5±0.625. In some embodiments, the delivery agent comprises Compound 236, DSPC, Cholesterol, Compound 428 or PEG-DMG, and SA3 or a salt thereof, e.g., with a mole ratio of about 45.8:10.5:36.8:1.4:5.5. In some embodiments, the delivery agent comprises Compound 18 or 236, DSPC, Cholesterol, Compound 428 or PEG-DMG, and SA3 or a salt thereof, e.g., with a mole ratio in the range of about 30 to about 60 mol % Compound 18 or 236 (or related suitable amino lipid) (e.g., 30-40, 40-45, 45-50, 50-55 or 55-60 mol % Compound 18 or 236 (or related suitable amino lipid)), about 5 to about 20 mol % phospholipid (or related suitable phospholipid or “helper lipid”) (e.g., 5-10, 10-15, or 15-20 mol % phospholipid (or related suitable phospholipid or “helper lipid”)), about 20 to about 50 mol % cholesterol (or related sterol or “non-cationic” lipid) (e.g., about 20-30, 30-35, 35-40, 40-45, or 45-50 mol % cholesterol (or related sterol or “non-cationic” lipid)), about 0.05 to about 10 mol % PEG lipid (or other suitable PEG lipid) (e.g., 0.05-1, 1-2, 2-3, 3-4, 4-5, 5-7, or 7-10 mol % PEG lipid (or other suitable PEG lipid)), and about 1 to about 10 mol % SA3 or a salt thereof (e.g., 1-3, 3-5, 5-7, 7-10, 3-8, 3.5-6.5 mol % SA3 or a salt thereof). An exemplary delivery agent can comprise mole ratios of, for example, 47.6:9.5:36.6:1.4:4.9, 47.3:9.5:36.4:1.4:5.5, or 45.8:10.5:36.8:1.4:5.5. In certain instances, an exemplary delivery agent can comprise mole ratios of, for example, 48:9.5:35.5:1.5:5.5; 47:10:36:1.5:5.5; 46:10.5:36.5:1.5:5.5; 45:10.5:37.5:1.5:5.5; 48:9.5:36:1.5:5; 47:10:36.5:1.5:5; 46:10.5:37:1.5:5; or 45:10.5:38:1.5:5. In some embodiments, the delivery agent comprises Compound 18 or 236, DSPC, Cholesterol, Compound 428 or PEG-DMG, and SA3 or a salt thereof, e.g., with a mole ratio of about 47.6:9.5:36.6:1.4:4.9. In some embodiments, the delivery agent comprises Compound 18 or 236, DSPC, Cholesterol, Compound 428 or PEG-DMG, and SA3 or a salt thereof, e.g., with a mole ratio of about 47.3:9.5:36.4:1.4:5.5. In some embodiments, the delivery agent comprises Compound 18 or 236, DSPC, Cholesterol, Compound 428 or PEG-DMG, and SA3 or a salt thereof, e.g., with a mole ratio of about 45.8:10.5:36.8:1.4:5.5.

In some embodiments, the polynucleotide (e.g., polynucleotide encoding an antigen) disclosed herein is formulated with a delivery agent comprising, e.g., a compound having the Formula (I), e.g., any of Compounds 1-232, e.g., Compound 18; a compound having the Formula (III), (IV), (V), or (VI), e.g., any of Compounds 233-342, e.g., Compound 236; or a compound having the Formula (VIII), e.g., any of Compounds 419-428, e.g., Compound 428, or any combination thereof. In some embodiments, the delivery agent comprises Compound 18, DSPC, Cholesterol, and Compound 428 or PEG-DMG, e.g., with a mole ratio of about 49.5±3:10.5±2:39±3:1±0.75. In some embodiments, the delivery agent comprises Compound 236, DSPC, Cholesterol, and Compound 428 or PEG-DMG, e.g., with a mole ratio of about 49.5±3:10.5±2:39±3:1±0.75. In some embodiments, the delivery agent comprises about 48-52 mol % Compound 18 or 236 (or related suitable amino lipid) (e.g., 48-51, 48-50, 49-52, or 49-51 mol % Compound 18 or 236 (or related suitable amino lipid)), about 9-12 mol % phospholipid (or related suitable phospholipid or “helper lipid”) (e.g., 9-11, 9-10, 10-12, 10-11.5, 10-11 mol % phospholipid (or related suitable phospholipid or “helper lipid”)), about 36-42 mol % cholesterol (or related sterol or “non-cationic”lipid) (e.g., about 36-41, 36-40, 37-40, or 38-40 mol % cholesterol (or related sterol or “non-cationic” lipid)) and about 0.25-2.5 mol % PEG lipid (or other suitable PEG lipid) (e.g., 0.25-2, 0.25-1.5, 0.25-2, or 0.5-1.5 mol % PEG lipid (or other suitable PEG lipid)).

In some embodiments, the polynucleotide (e.g., polynucleotide encoding an antigen) disclosed herein is formulated with a delivery agent comprising, e.g., a compound having the Formula (I), e.g., any of Compounds 1-232, e.g., Compound 18; a compound having the Formula (III), (IV), (V), or (VI), e.g., any of Compounds 233-342, e.g., Compound 236; a compound having the Formula (VIII), e.g., any of Compounds 419-428, e.g., Compound 428, or a compound having the Formula A1, A2, A3, A4, or A5, e.g., any one of SA1-SA41, or any combination thereof. In some embodiments, the delivery agent comprises Compound 18, DSPC, Cholesterol, and Compound 428 or PEG-DMG, e.g., with a mole ratio of about 46.5±3:10±2:36±3:1.25±0.75:4.5±1.5. In some embodiments, the delivery agent comprises Compound 236, DSPC, Cholesterol, and Compound 428 or PEG-DMG, e.g., with a mole ratio of about 46.5±3:10±2:36±3:1.25±0.75:4.5±1.5. In some embodiments, the delivery agent comprises about 43-49 mol % Compound 18 or 236 (or related suitable amino lipid) (e.g., 43-48, 44-48, 45-48, or 45.5-48 mol % Compound 18 or 236 (or related suitable amino lipid)), about 8-12 mol % phospholipid (or related suitable phospholipid or “helper lipid”) (e.g., 8-11, 8-10, 9-12, 9-11, 9.5-10.5 mol % phospholipid (or related suitable phospholipid or “helper lipid”)), about 33-39 mol % cholesterol (or related sterol or “non-cationic” lipid) (e.g., about 33-38, 34-38, 35-38, or 36-37 mol % cholesterol (or related sterol or “non-cationic” lipid)), about 0.5-2 mol % PEG lipid (or other suitable PEG lipid) (e.g., 0.5-1.5, 0.75-1.5, or 1-1.5 mol % PEG lipid (or other suitable PEG lipid)), and about 3-6 mol % cationic agent (e.g., sterol amine) (e.g., 3-5, 3-4.5, 4-6, or 5-6 mol % cationic agent (e.g., sterol amine)).

In some embodiments, the polynucleotide (e.g., polynucleotide encoding an antigen) disclosed herein is formulated with a delivery agent comprising, e.g., a compound having the Formula (I), e.g., any of Compounds 1-232, e.g., Compound 18; a compound having the Formula (III), (IV), (V), or (VI), e.g., any of Compounds 233-342, e.g., Compound 236; a compound having the Formula (VIII), e.g., any of Compounds 419-428, e.g., Compound 428, or a compound having the Formula A1, A2, A3, A4, or A5, e.g., any one of SA1-SA41, or any combination thereof. In some embodiments, the delivery agent comprises Compound 18, DSPC, Cholesterol, and Compound 428 or PEG-DMG, e.g., with a mole ratio of about 47±3:10±2:36±3:1.25±0.75:4.5±1.5. In some embodiments, the delivery agent comprises Compound 236, DSPC, Cholesterol, and Compound 428 or PEG-DMG, e.g., with a mole ratio of about 46.5±3:10±2:36±3:1.25±0.75:4.5±1.5. In some embodiments, the delivery agent comprises about 43-49 mol % Compound 18 or 236 (or related suitable amino lipid) (e.g., 43-48, 44-48, 45-48, or 45.5-48 mol % Compound 18 or 236 (or related suitable amino lipid)), about 8-12 mol % phospholipid (or related suitable phospholipid or “helper lipid”) (e.g., 8-11, 8-10, 9-12, 9-11, 9.5-10.5 mol % phospholipid (or related suitable phospholipid or “helper lipid”)), about 33-39 mol % cholesterol (or related sterol or “non-cationic” lipid) (e.g., about 33-38, 34-38, 35-38, or 36-37 mol % cholesterol (or related sterol or “non-cationic” lipid)), about 0.5-2 mol % PEG lipid (or other suitable PEG lipid) (e.g., 0.5-1.5, 0.75-1.5, or 1-1.5 mol % PEG lipid (or other suitable PEG lipid)), and about 3-6 mol % cationic agent (e.g., sterol amine) (e.g., 3-5, 3-4.5, 4-6, or 5-6 mol % cationic agent (e.g., sterol amine)). In some embodiments, the delivery agent comprises Compound 18, DSPC, Cholesterol, DMG-PEG-2k, and SA3. In further embodiments, the delivery agent comprises about 45-48 mol % Compound 18, about 9-11 mol % DSPC, about 35-38 mol % cholesterol, about 1-3 mol % DMG-PEG-2k, and about 4-6 mol % SA3. In further embodiments, the delivery agent comprises about 45-48 mol % Compound 18, about 9-11 mol % DSPC, about 35-38 mol % cholesterol, about 1-3 mol % DMG-PEG-2k, and about 4-6 mol % SA3. In further embodiments, the delivery agent comprises about 45.8-47.6 mol % Compound 18, about 9.5-10.5 mol % DSPC, about 36.4-36.8 mol % cholesterol, about 1.4 mol % DMG-PEG-2k, and about 4.9-5.5 mol % SA3.

Unless otherwise specified, mole ratios/percentages described herein refer to the composition for delivery and do not refer to the cargo (e.g., nucleic acid therapeutic, e.g., polynucleotide, e.g., mRNA).

### Payload Molecules

The compositions of the disclosure can be used to deliver a wide variety of different agents to an airway cell. An airway cell can be a cell lining the respiratory tract, e.g., in the mouth, nose, throat, or lungs. The therapeutic agent is capable of mediating (e.g., directly mediating or via a bystander effect) a therapeutic effect in such an airway cell. Typically the therapeutic agent delivered by the composition is a nucleic acid, although non-nucleic acid agents, such as small molecules, chemotherapy drugs, peptides, polypeptides and other biological molecules are also encompassed by the disclosure. Nucleic acids that can be delivered include DNA-based molecules (i.e., comprising deoxyribonucleotides) and RNA-based molecules (i.e., comprising ribonucleotides). Furthermore, the nucleic acid can be a naturally occurring form of the molecule or a chemically-modified form of the molecule (e.g., comprising one or more modified nucleotides).

### Agents for Enhancing Protein Expression

In one embodiment, the therapeutic agent is an agent that enhances (i.e., increases, stimulates, upregulates) protein expression. Non-limiting examples of types of therapeutic agents that can be used for enhancing protein expression include RNAs, mRNAs, dsRNAs, CRISPR/Cas9 technology, ssDNAs and DNAs (e.g., expression vectors).

In one embodiment, the therapeutic agent is a DNA therapeutic agent. The DNA molecule can be a double-stranded DNA, a single-stranded DNA (ssDNA), or a molecule that is a partially double-stranded DNA, i.e., has a portion that is double-stranded and a portion that is single-stranded. In some cases, the DNA molecule is triple-stranded or is partially triple-stranded, i.e., has a portion that is triple stranded and a portion that is double stranded. The DNA molecule can be a circular DNA molecule or a linear DNA molecule.

A DNA therapeutic agent can be a DNA molecule that is capable of transferring a gene into a cell, e.g., that encodes and can express a transcript. For example, the DNA therapeutic agent can encode a protein of interest, to thereby increase expression of the protein of interest in an airway upon delivery by an LNP. In some embodiments, the DNA molecule can be naturally-derived, e.g., isolated from a natural source. In other embodiments, the DNA molecule is a synthetic molecule, e.g., a synthetic DNA molecule produced in vitro. In some embodiments, the DNA molecule is a recombinant molecule. Non-limiting exemplary DNA therapeutic agents include plasmid expression vectors and viral expression vectors.

The DNA therapeutic agents described herein, e.g., DNA vectors, can include a variety of different features. The DNA therapeutic agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

In some embodiments, the payload comprises a genetic modulator, i.e., at least one component of a system which modifies a nucleic acid sequence in a DNA molecule, e.g., by altering a nucleobase, e.g., introducing an insertion, a deletion, a mutation (e.g., a missense mutation, a silent mutation or a nonsense mutation), a duplication, or an inversion, or any combination thereof. In some embodiments, the genetic modulator comprises a DNA base editor, CRISPR/Cas gene editing system, a zinc finger nuclease (ZFN) system, a Transcription activator-like effector nuclease (TALEN) system, a meganuclease system, or a transposase system, or any combination thereof.

In some embodiments, the genetic modulator comprises a template DNA. In some embodiments, the genetic modulator does not comprise a template DNA. In some embodiments, the genetic modulator comprises a template RNA. In some embodiments, the genetic modulator does not comprise a template RNA.

In some embodiments, the genetic modulator is a CRISPR/Cas gene editing system. In some embodiments, the CRISPR/Cas gene editing system comprises a guide RNA (gRNA) molecule comprising a targeting sequence specific to a sequence of a target gene and a peptide having nuclease activity, e.g., endonuclease activity, e.g., a Cas protein or a fragment (e.g., biologically active fragment) or a variant thereof, e.g., a Cas9 protein, a fragment (e.g., biologically active fragment) or a variant thereof; a Cas3 protein, a fragment (e.g., biologically active fragment) or a variant thereof; a Cas12a protein, a fragment (e.g., biologically active fragment) or a variant thereof; a Cas 12e protein, a fragment (e.g., biologically active fragment) or a variant thereof; a Cas 13 protein, a fragment (e.g., biologically active fragment) or a variant thereof; or a Cas14 protein, a fragment (e.g., biologically active fragment) or a variant thereof.

In some embodiments, the CRISPR/Cas gene editing system comprises a gRNA molecule comprising a targeting sequence specific to a sequence of a target gene, and a nucleic acid encoding a peptide having nuclease activity, e.g., endonuclease activity, e.g., a Cas protein or a fragment (e.g., biologically active fragment) or variant thereof, e.g., a Cas9 protein, a fragment (e.g., biologically active fragment) or a variant thereof; a Cas3 protein, a fragment (e.g., biologically active fragment) or a variant thereof; a Cas12a protein, a fragment (e.g., biologically active fragment) or a variant thereof; a Cas12e protein, a fragment (e.g., biologically active fragment) or a variant thereof; a Cas13 protein, a fragment (e.g., biologically active fragment) or a variant thereof; or a Cas14 protein, a fragment (e.g., biologically active fragment) or a variant thereof.

In some embodiments, the CRISPR/Cas gene editing system comprises a nucleic acid encoding a gRNA molecule comprising a targeting sequence specific to a sequence of a target gene, and a Cas9 protein, a fragment (e.g., biologically active fragment) or a variant thereof.

In some embodiments, the CRISPR/Cas gene editing system comprises a nucleic acid encoding a gRNA molecule comprising a targeting sequence specific to a sequence of a target gene, and a nucleic acid encoding a Cas9 protein, a fragment (e.g., biologically active fragment) or a variant thereof.

In some embodiments, the CRISPR/Cas gene editing system further comprises a template DNA. In some embodiments, the CRISPR/Cas gene editing system further comprises a template RNA. In some embodiments, the CRISPR/Cas gene editing system further comprises a Reverse transcriptase.

In some embodiments of any of the methods, compositions, or cells disclosed herein, the genetic modulator is a zinc finger nuclease (ZFN) system. In some embodiments, the ZFN system comprises a peptide having: a Zinc finger DNA binding domain, a fragment (e.g., biologically active fragment) or a variant thereof; and/or nuclease activity, e.g., endonuclease activity. In some embodiments, the ZFN system comprises a peptide having a Zn finger DNA binding domain. In some embodiments, the Zn finger binding domain comprises 1, 2, 3, 4, 5, 6, 7, 8 or more Zinc fingers. In some embodiments, the ZFN system comprises a peptide having nuclease activity e.g., endonuclease activity. In some embodiments, the peptide having nuclease activity is a type-IL restriction 1-like endonuclease, e.g., a FokI endonuclease. In some embodiments, the ZFN system comprises a nucleic acid encoding a peptide having: a Zinc finger DNA binding domain, a fragment (e.g., biologically active fragment) or a variant thereof; and/or nuclease activity, e.g., endonuclease activity.

In some embodiments, the ZFN system comprises a nucleic acid encoding a peptide having a Zn finger DNA binding domain. In some embodiments, the Zn finger binding domain comprises 1, 2, 3, 4, 5, 6, 7, 8 or more Zinc fingers. In some embodiments, the ZFN system comprises a nucleic acid encoding a peptide having nuclease activity e.g., endonuclease activity. In some embodiments, the peptide having nuclease activity is a type-IL restriction 1-like endonuclease, e.g., a FokI endonuclease.

In some embodiments, the system further comprises a template, e.g., template DNA.

In some embodiments of any of the methods, compositions, or cells disclosed herein, the genetic modulator is a Transcription activator-like effector nuclease (TALEN) system. In some embodiments, the system comprises a peptide having: a Transcription activator-like (TAL) effector DNA binding domain, a fragment (e.g., biologically active fragment) or a variant thereof; and/or nuclease activity, e.g., endonuclease activity. In some embodiments, the system comprises a peptide having a TAL effector DNA binding domain, a fragment (e.g., biologically active fragment) or a variant thereof. In some embodiments, the system comprises a peptide having nuclease activity, e.g., endonuclease activity. In some embodiments, the peptide having nuclease activity is a type-II restriction 1-like endonuclease, e.g., a FokI endonuclease.

In some embodiments, the system comprises a nucleic acid encoding a peptide having: a Transcription activator-like (TAL) effector DNA binding domain, a fragment (e.g., biologically active fragment) or a variant thereof; and/or nuclease activity, e.g., endonuclease activity. In some embodiments, the system comprises a nucleic acid encoding a peptide having a Transcription activator-like (TAL) effector DNA binding domain, a fragment (e.g., biologically active fragment) or a variant thereof. In some embodiments, the system comprises a nucleic acid encoding a peptide having nuclease activity, e.g., endonuclease activity. In some embodiments, the peptide having nuclease activity is a type-II restriction 1-like endonuclease, e.g., a FokI endonuclease.

In some embodiments, the system further comprises a template, e.g., a template DNA.

In some embodiments of any of the methods, compositions, or cells disclosed herein, the genetic modulator is a meganuclease system. In some embodiments, the meganuclease system comprises a peptide having a DNA binding domain and nuclease activity, e.g., a homing endonuclease. In some embodiments, the homing endonuclease comprises a LAGLIDADG endonuclease, GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.

In some embodiments, the meganuclease system comprises a nucleic acid encoding a peptide having a DNA binding domain and nuclease activity, e.g., a homing endonuclease. In some embodiments, the homing endonuclease comprises a LAGLIDADG endonuclease, GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.

In some embodiments, the system further comprises a template, e.g., a template DNA.

In some embodiments of any of the methods, compositions, or cells disclosed herein, the genetic modulator is a transposase system. In some embodiments, the transposase system comprises a nucleic acid sequence encoding a peptide having reverse transcriptase and/or nuclease activity, e.g., a retrotransposon, e.g., an LTR retrotransposon or a non-LTR retrotransposon. In some embodiments, the transposase system comprises a template, e.g., an RNA template.

In one embodiment, the therapeutic agent is an RNA therapeutic agent. The RNA molecule can be a single-stranded RNA, a double-stranded RNA (dsRNA) or a molecule that is a partially double-stranded RNA, i.e., has a portion that is double-stranded and a portion that is single-stranded. The RNA molecule can be a circular RNA molecule or a linear RNA molecule.

An RNA therapeutic agent can be an RNA therapeutic agent that is capable of transferring a gene into a cell, e.g., encodes a protein of interest, to thereby increase expression of the protein of interest in an airway cell. In some embodiments, the RNA molecule can be naturally-derived, e.g., isolated from a natural source. In other embodiments, the RNA molecule is a synthetic molecule, e.g., a synthetic RNA molecule produced in vitro.

Non-limiting examples of RNA therapeutic agents include messenger RNAs (mRNAs) (e.g., encoding a protein of interest), modified mRNAs (mmRNAs), mRNAs that incorporate a micro-RNA binding site(s) (miR binding site(s)), modified RNAs that comprise functional RNA elements, microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNA), locked nucleic acids (LNAs) and that encode components of CRISPR/Cas9 technology, each of which is described further in subsections below. In some embodiments, the RNA modulator comprises an RNA base editor system. In some embodiments, the RNA base editor system comprises: a deaminase, e.g., an RNA-specific adenosine deaminase (ADAR); a Cas protein, a fragment (e.g., biologically active fragment) or a variant thereof; and/or a guide RNA. In some embodiments, the RNA base editor system further comprises a template, e.g., a DNA or RNA template.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (tm5U), 1-taurinomethyl-pseudouridine, 5-taurinomethyl-2-thio-uridine(tm5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQO), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In one embodiment, the modified nucleobase is N1-methylpseudouridine (m1ψ) and the mRNA of the disclosure is fully modified with N1-methylpseudouridine (m1ψ). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m1ψ), 5-methoxy-uridine (mo5U), 5-methyl-cytidine (m5C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

### Therapeutic Agents for Reducing Protein Expression

In one embodiment, the therapeutic agent is a therapeutic agent that reduces (i.e., decreases, inhibits, downregulates) protein expression. In one embodiment, the therapeutic agent reduces protein expression in the target airway cell Non-limiting examples of types of therapeutic agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

### Peptide/Polypeptide Therapeutic Agents

In one embodiment, the therapeutic agent is a peptide therapeutic agent. In one embodiment the therapeutic agent is a polypeptide therapeutic agent.

In some embodiments, the therapeutic payload or prophylactic payload comprises an mRNA encoding: a secreted protein; a membrane-bound protein; or an intercellular protein, or peptides, polypeptides or biologically active fragments thereof.

In some embodiments, the therapeutic payload or prophylactic payload comprises an mRNA encoding a secreted protein, a peptide, a polypeptide or a biologically active fragment thereof. In some embodiments, the therapeutic payload or prophylactic payload comprises an mRNA encoding a membrane-bound protein, a peptide, a polypeptide or a biologically active fragment thereof. In some embodiments, the therapeutic payload or prophylactic payload comprises an mRNA encoding an intracellular protein, a peptide, a polypeptide or a biologically active fragment thereof. In some embodiments, the therapeutic payload or prophylactic payload comprises a protein, polypeptide, or peptide. In some embodiments, the peptide therapeutic agent is used to treat an autoimmune disease associated with the mucosa, such as ulcerative colitis or Crohn's disease. In some embodiments, the polypeptide therapeutic agent is not cystic fibrosis transmembrane regulator (CFTR).

In some embodiments, the peptide or polypeptide is naturally-derived, e.g., isolated from a natural source. In other embodiments, the peptide or polypeptide is a synthetic molecule, e.g., a synthetic peptide or polypeptide produced in vitro. In some embodiments, the peptide or polypeptide is a recombinant molecule. In some embodiments, the peptide or polypeptide is a chimeric molecule. In some embodiments, the peptide or polypeptide is a fusion molecule. In one embodiment, the peptide or polypeptide therapeutic agent of the composition is a naturally occurring peptide or polypeptide. In one embodiment, the peptide or polypeptide therapeutic agent of the composition is a modified version of a naturally occurring peptide or polypeptide (e.g., contains less than 3, less than 5, less than 10, less than 15, less than 20, or less than 25 amino substitutions, deletions, or additions compared to its wild type, naturally occurring peptide or polypeptide counterpart).

### LNPs Comprising Cationic Agents

The LNPs of the invention comprise a LNP core and a cationic agent disposed primarily on the outer surface of the core. Such LNPs have a greater than neutral zeta potential at physiologic pH.

Core lipid nanoparticles typically comprise one or more of the following components: lipids (which may include ionizable amino lipids, phospholipids, helper lipids which may be neutral lipids, zwitterionic lipid, anionic lipids, and the like), structural lipids such as cholesterol or cholesterol analogs, fatty acids, polymers, stabilizers, salts, buffers, solvent, and the like.

Certain of the LNP cores provided herein comprise an ionizable lipid, such as an ionizable lipid, e.g., an ionizable amino lipid, a phospholipid, a structural lipid, and optionally a stabilizer (e.g., a molecule comprising polyethylene glycol) which may or may not be provided conjugated to another lipid.

The structural lipid may be but is not limited to a sterol such as for example cholesterol. The structural lipid can be β-sitosterol.

The helper lipid is a non-cationic lipid. The helper lipid may comprise at least one fatty acid chain of at least 8C and at least one polar headgroup moiety.

When a molecule comprising polyethylene glycol (i.e. PEG) is used, it may be used as a stabilizer. In some embodiments, the molecule comprising polyethylene glycol may be polyethylene glycol conjugated to a lipid and thus may be provided as PEG-c-DOMG or PEG-DMG, for example. Certain of the LNPs provided herein comprise no or low levels of PEGylated lipids, including no or low levels of alkyl-PEGylated lipids, and may be referred to herein as being free of PEG or PEGylated lipid. Thus, some LNPs comprise less than 0.5 mol % PEGylated lipid. In some instances, PEG may be an alkyl-PEG such as methoxy-PEG. Still other LNPs comprise non-alkyl-PEG such as hydroxy-PEG, and/or non-alkyl-PEGylated lipids such as hydroxy-PEGylated lipids. Certain LNPs provided herein comprise high levels of PEGylated lipids. Some LNPS comprise 0.5 mol % PEGylated lipid. Some LNPs comprise more than 0.5 mol % PEGylated lipid. In some embodiments, the LNPs comprise 1.5 mol % PEGylated lipid. In some embodiments, the LNPs comprise 3.0 mol % PEGylated lipid. In some embodiments, the LNPs comprise 0.1 mol % to 3.0 mol % PEGylated lipid, 0.5 mol % to 2.0 mol % PEGylated lipid, or 1.0 mol % to 1.5 mol % PEGylated lipid.

In some embodiments, a core nanoparticle composition can have the formulation of Compound 18:Phospholipid:Chol: N-lauroyl-D-erythro-sphinganylphosphorylcholine with a mole ratio of 50:10:38.5:1.5. In some embodiments, a nanoparticle core composition can have the formulation of Compound 18:DSPC:Chol:Compound 428 with a mole ratio of 50:10:38.5:1.5.

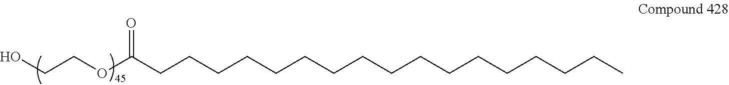

Nanoparticles of the present disclosure comprise at least one compound according to Formula (I). For example, the nanoparticle composition can include one or more of Compounds 1-147. Nanoparticles can also include a variety of other components. For example, the nanoparticle composition can include one or more other lipids in addition to a lipid according to Formula (I) or (II), for example (i) at least one phospholipid, (ii) at least one structural lipid, (iii) at least one PEG-lipid, or (iv) any combination thereof.

In some embodiments, the nanoparticle composition comprises a compound of Formula (I), (e.g., Compounds 18, 25, 26 or 48). In some embodiments, the nanoparticle composition comprises a compound of Formula (I) (e.g., Compounds 18, 25, 26 or 48) and a phospholipid (e.g., DSPC, DOPE, or MSPC). In some embodiments, the nanoparticle composition comprises a compound of Formula (I) (e.g., Compounds 18, 25, 26 or 48) and a phospholipid (e.g., DSPC, DPPC, DOPE, or MSPC).

The present disclosure also provides process of preparing a nanoparticle comprising contacting a lipid nanoparticle with a cationic agent, wherein the lipid nanoparticle comprises:
(a) a lipid nanoparticle core comprising:
   (i) an ionizable lipid,
   (ii) a phospholipid,
   (iii) a structural lipid, and
   (iv) a PEG-lipid, and
(b) a polynucleotide (e.g., polynucleotide encoding an antigen) encapsulated within the core for delivery into a cell.

In some embodiments, the contacting of the lipid nanoparticle with a cationic agent comprises dissolving the cationic agent in a non-ionic excipient. In some embodiments, the non-ionic excipient is selected from macrogol 15 hydroxystearate (HS 15), 1,2-dimyristoyl-rac-glycero-3-methoxypolyethylene glycol-2000 (DMG-PEG2K), Compound 428, polyoxyethylene sorbitan monooleate [TWEEN®80], and d-α-Tocopherol polyethylene glycol succinate (TPGS). In some embodiments, the non-ionic excipient is macrogol 15 hydroxystearate (HS 15). In some embodiments, the contacting of the lipid nanoparticle with a cationic agent comprises the cationic agent dissolved in a buffer solution. In some embodiments, the buffer solution is a phosphate buffered saline (PBS). In some embodiments, the buffer solution is a Tris-based buffer.

Provided are nanoparticles prepared by the process as described herein, e.g., by contacting the lipid nanoparticle with a cationic agent. In some embodiments, the cationic agent can be a sterol amine such as SA3. In some embodiments, the lipid nanoparticle core of the lipid nanoparticle optionally comprises a PEG-lipid. In some embodiments, the lipid nanoparticle core forming the lipid nanoparticle which is contacted with the cationic agent is substantially free of PEG-lipid. In some embodiments, the PEG-lipid is added to the lipid nanoparticle together with the cationic agent, prior to the contacting with the cationic agent, or after the contacting with the cationic agent.

In one embodiment, an LNP of the invention can be made using traditional mixing technology in which the polynucleotide is mixed with core LNP components to create the core LNP plus payload. Once this loaded core LNP is prepared, the cationic agent is contacted with the loaded core LNP.

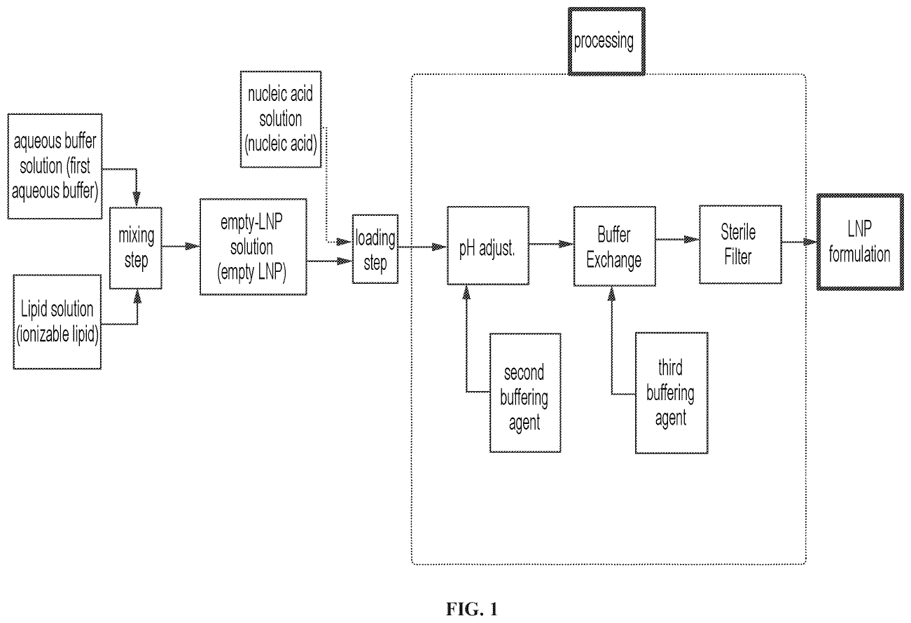

In another embodiment, an LNP of the invention can be made using empty LNPs as the starting point. For example, as shown in FIG. 1, empty LNPs are made prior to loading in the polynucleotide. Once the polynucleotide is contacted with the LNP, the cationic agent can be added to form an LNP of the invention.

For example, in one embodiment, in the post-hoc loading (PHL) method, empty LNPs are formulated first in a nanoprecipitation step, and buffer exchanged into a low pH buffer (i.e. pH 5). Next, these empty LNPs are introduced to mRNA (also acidified at low pH) through a mixing event. After the mixing step, a pH adjustment method is used to neutralize the pH. Finally, a PEG lipid, e.g., DMG-PEG-2k is added to stabilize the particle. These particles are then concentrated to the target concentration and filtered. A cationic agent, e.g., SA3 is added.

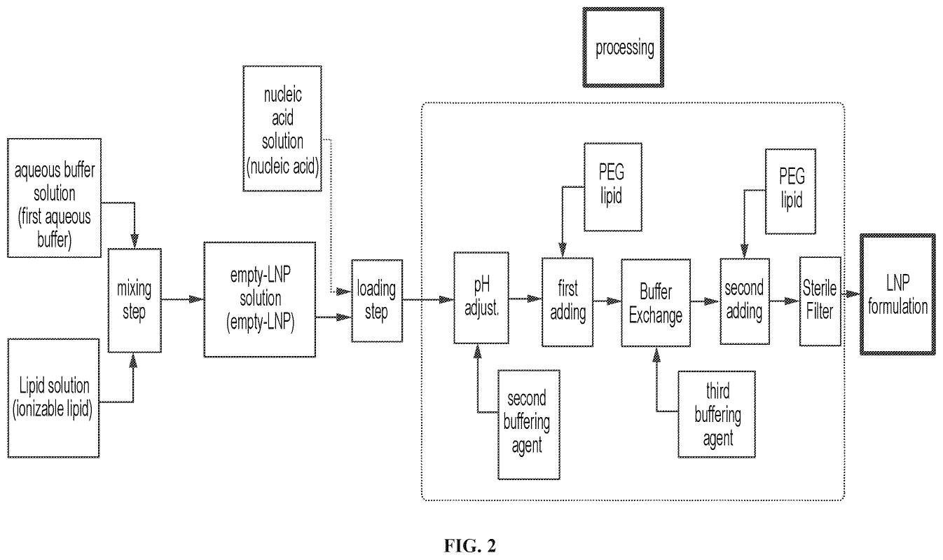

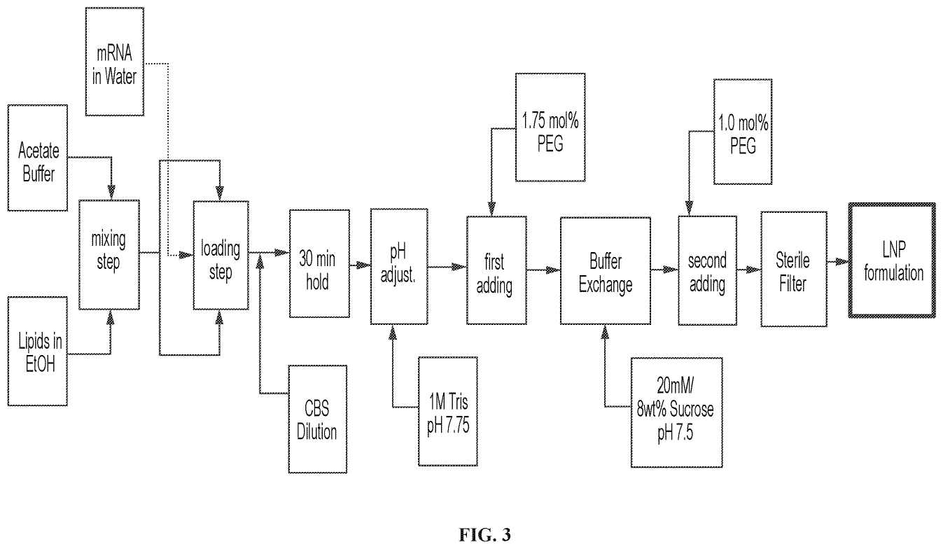

A variation of the empty LNP starting point is illustrated in FIG. 2. FIG. 2 shows that the lipids of the LNP, excluding the PEG lipids, are used to form an empty LNP. The nucleic acid solution is then contacted with the empty LNPs, forming loaded LNPs. The PEG lipids are added at one or two points during further processing of the loaded LNPs and the cationic agent can be added at any point during that further processing, illustrated by the dotted box in FIG. 2. FIG. 3 is a more specific version of the process in FIG. 2 and, again, the cationic agent can be added at any point during the further processing of the loaded LNP.

In some embodiments, an LNP of the invention can be prepared using nanoprecipitation, which is the unit operation in which the LNPs are self-assembled from their individual lipid components by way of kinetic mixing and subsequent maturation and continuous dilution. This unit operation includes three individual steps, which are: mixing of the aqueous and organic inputs, maturation of the LNPs, and dilution after a controlled residence time. Due to the continuous nature of these steps, they are considered one unit operation. The unit operation includes the continuous inline combination of three liquid streams with one inline maturation step: mixing of the aqueous buffer with lipid stock solution, maturation via controlled residence time, and dilution of the nanoparticles. The nanoprecipitation itself occurs in the scale-appropriate mixer, which is designed to allow continuous, high-energy, combination of the aqueous solution with the lipid stock solution dissolved in ethanol. The aqueous solution and the lipid stock solution both flow simultaneously into the mixing hardware continuously throughout this operation. The ethanol content, which keeps the lipids dissolved, is abruptly reduced and the lipids all precipitate with each other. The particles are thus self-assembled in the mixing chamber.

One of the objectives of unit operation is to exchange the solution into a fully aqueous buffer, free of ethanol, and to reach a target concentration of LNP. This can be achieved by first reaching a target processing concentration, then diafiltering, and then (if necessary) a final concentration step once the ethanol has been completely removed.

In some embodiments, an LNP of the invention can be prepared using nanoprecipitation, which is the unit operation in which the LNPs are self-assembled from their individual lipid components by way of kinetic mixing and subsequent maturation and continuous dilution. This unit operation includes three individual steps, which are: mixing of the aqueous and organic inputs, maturation of the LNPs, and dilution after a controlled residence time. Due to the continuous nature of these steps, they are considered one unit operation. The unit operation includes the continuous inline combination of three liquid streams with one inline maturation step: mixing of the aqueous buffer with lipid stock solution, maturation via controlled residence time, and dilution of the nanoparticles. The nanoprecipitation itself occurs in the scale-appropriate mixer, which is designed to allow continuous, high-energy, combination of the aqueous solution with the lipid stock solution dissolved in ethanol. The aqueous solution and the lipid stock solution both flow simultaneously into the mixing hardware continuously throughout this operation. The ethanol content, which keeps the lipids dissolved, is abruptly reduced and the lipids all precipitate with each other. The particles are thus self-assembled in the mixing chamber.

One of the objectives of unit operation is to exchange the solution into a fully aqueous buffer, free of ethanol, and to reach a target concentration of LNP. This can be achieved by first reaching a target processing concentration, then diafiltering, and then (if necessary) a final concentration step once the ethanol has been completely removed.

In some aspects, the present disclosure provides a method of preparing an empty-lipid nanoparticle solution (empty-LNP solution) comprising an empty lipid nanoparticle (empty LNP), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time; and
   i-c) adding a diluting solution to the intermediate empty-LNP solution, thereby forming the empty-LNP solution comprising the empty LNP.

In some aspects, the present disclosure provides a method of preparing an empty-lipid nanoparticle solution (empty-LNP solution) comprising an empty lipid nanoparticle (empty LNP), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time;
   i-c) adding a diluting solution to the intermediate empty-LNP solution, thereby forming the empty-LNP solution comprising the empty LNP; and
ii) processing the empty-LNP solution.

In some aspects, the present disclosure provides a method of preparing an empty-lipid nanoparticle solution (empty-LNP solution) comprising an empty lipid nanoparticle (empty LNP), comprising:
ii) processing an empty-LNP solution comprising the empty LNP.

In some aspects, the present disclosure provides a method of preparing a lipid nanoparticle formulation (LNP formulation), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time;
   i-c) adding a diluting solution to the intermediate empty-LNP solution, thereby forming the empty-LNP solution comprising the empty LNP; and
ii) processing the empty-LNP solution; and
iii) a loading step, comprising mixing a nucleic acid solution comprising a nucleic acid with the empty-LNP solution, thereby forming a loaded LNP solution comprising a loaded lipid nanoparticle (loaded LNP).

In some aspects, the present disclosure provides a method of preparing a lipid nanoparticle formulation (LNP formulation), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time;
   i-c) adding a diluting solution to the intermediate empty-LNP solution, thereby forming the empty-LNP solution comprising the empty LNP; and
ii) processing the empty-LNP solution;
iii) a loading step, comprising mixing a nucleic acid solution comprising a nucleic acid with the empty-LNP solution, thereby forming a loaded LNP solution comprising a loaded lipid nanoparticle (loaded LNP); and
iv) processing the loaded LNP solution, thereby forming the loaded LNP formulation.

In some aspects, the present disclosure provides a method of preparing a lipid nanoparticle formulation (LNP formulation), comprising:
i) a nanoprecipitation step, comprising:
   i-a) mixing step, comprising mixing a lipid solution comprising an ionizable lipid, a structural lipid, a phospholipid, and a PEG lipid, with an aqueous buffer solution comprising a first buffering agent, thereby forming an intermediate empty-lipid nanoparticle solution (intermediate empty-LNP solution) comprising an intermediate empty nanoparticle (intermediate empty LNP);
   i-b) holding the intermediate empty-LNP solution for a residence time;
   i-c) adding a diluting solution to the intermediate empty-LNP solution, thereby forming the empty-LNP solution comprising the empty LNP; and
ii) processing the empty-LNP solution;
iii) a loading step, comprising mixing a nucleic acid solution comprising a nucleic acid with the empty-LNP solution, thereby forming a loaded LNP solution comprising a loaded lipid nanoparticle (loaded LNP);
iv) processing the loaded LNP solution, thereby forming the loaded LNP formulation; and
v) adding a cationic agent.

In some aspects, the present disclosure provides a method of preparing a lipid nanoparticle formulation (LNP formulation), comprising:
iii) a loading step, comprising mixing a nucleic acid solution comprising a nucleic acid with an empty-LNP solution comprising an empty LNP, thereby forming a loaded nanoparticle solution (loaded LNP solution) comprising a loaded lipid nanoparticle (loaded LNP).

In some aspects, the present disclosure provides a method of preparing a lipid nanoparticle formulation (LNP formulation), comprising:
iii) a loading step, comprising mixing a nucleic acid solution comprising a nucleic acid with an empty-LNP solution comprising an empty LNP, thereby forming a loaded nanoparticle solution (loaded LNP solution) comprising a loaded lipid nanoparticle (loaded LNP); and
iv) processing the loaded LNP solution, thereby forming the loaded LNP formulation.

In some aspects, the present disclosure provides a method of preparing a lipid nanoparticle formulation (LNP formulation), comprising:
iii) a loading step, comprising mixing a nucleic acid solution comprising a nucleic acid with an empty-LNP solution comprising an empty LNP, thereby forming a loaded nanoparticle solution (loaded LNP solution) comprising a loaded lipid nanoparticle (loaded LNP)
iv) processing the loaded LNP solution, thereby forming the loaded LNP formulation; and
v) adding a cationic agent.

In some embodiments, steps i-a) to i-c) are performed in separate operation units (e.g., separate reaction devices).

In some embodiments, steps i-a) to i-c) are performed in a single operation unit. In some embodiments, steps i-a) to i-c) are performed in a continuous flow device, such that step i-c) is downstream from step i-b) which is downstream from step i-a).

In some embodiments, in step i-c), the diluting solution is added once.

In some embodiments, in step i-c), the diluting solution is added continuously.

In some aspects, the present disclosure provides a method of producing an empty lipid nanoparticle (empty LNP), the method comprising: i) a mixing step, comprising mixing an ionizable lipid with a first buffering agent, thereby forming the empty LNP, wherein the empty LNP comprises from about 0.1 mol % to about 0.5 mol % of a polymeric lipid (for example, a PEG lipid).

In some aspects, the present disclosure provides a method of preparing an empty-lipid nanoparticle solution (empty-LNP solution) comprising an empty lipid nanoparticle (empty LNP), comprising:
i) a mixing step, comprising mixing a lipid solution comprising an ionizable lipid, a structural lipid, a phospholipid, and a PEG lipid, with an aqueous buffer solution comprising a first buffering agent, thereby forming an empty-lipid nanoparticle solution (empty-LNP solution) comprising the empty LNP.

In some aspects, the present disclosure provides a method of preparing an empty-lipid nanoparticle solution (empty-LNP solution) comprising an empty lipid nanoparticle (empty LNP), comprising:
i) a mixing step, comprising mixing a lipid solution comprising an ionizable lipid, a structural lipid, a phospholipid, and a PEG lipid, with an aqueous buffer solution comprising a first buffering agent, thereby forming an empty-lipid nanoparticle solution (empty-LNP solution) comprising the empty LNP; and
ii) processing the empty-LNP solution.

In some embodiments, the mixing step comprises mixing a lipid solution comprising the ionizable lipid with an aqueous buffer solution comprising the first buffering agent, thereby forming an empty-lipid nanoparticle solution (empty-LNP solution) comprising the empty LNP.

In some aspects, the present disclosure provides a method of preparing a loaded lipid nanoparticle (loaded LNP) associated with a nucleic acid, comprising: ii) a loading step, comprising mixing a nucleic acid with an empty LNP followed by addition of a cationic agent, thereby forming the loaded LNP.

In some embodiments, the loading step comprises mixing the nucleic acid solution comprising the nucleic acid with the empty-LNP solution followed by addition of a cationic agent, thereby forming a loaded lipid nanoparticle solution (loaded-LNP solution) comprising the loaded LNP.

In some embodiments, the empty LNP or the empty-LNP solution is subjected to the loading step without holding or storage.

In some embodiments, the empty LNP or the empty-LNP solution is subjected to the loading step after holding for a period of time.

In some embodiments, the empty LNP or the empty-LNP solution is subjected to the loading step after holding for about 1 minute, about 2 minutes, about 3 minutes, about 4 minutes, about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, or about 24 hours.

In some embodiments, the empty LNP or the empty-LNP solution is subjected to the loading step after storage for about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, or about 5 years.

In some embodiments, upon formation, the empty LNP or the empty-LNP solution is subjected to the loading step without storage or holding for a period of time.

In some aspects, the present disclosure provides a method, further comprising: ii) processing the empty-LNP solution.

In some aspects, the present disclosure provides a method, further comprising: iv) processing the loaded-LNP solution, thereby forming a lipid nanoparticle formulation (LNP formulation).

In contrast to other techniques for production (e.g., thin film rehydration/extrusion), ethanol-drop precipitation has been the industry standard for generating nucleic acid lipid nanoparticles. Precipitation reactions are favored due to their continuous nature, scalability, and ease of adoption. Those processes usually use high energy mixers (e.g., T-junction, confined impinging jets, microfluidic mixers, vortex mixers) to introduce lipids (in ethanol) to a suitable anti-solvent (i.e. water) in a controllable fashion, driving liquid supersaturation and spontaneous precipitation into lipid particles. In some embodiments, the vortex mixers used are those described in U.S. Patent Application Nos. 62/799,636 and 62/886,592, which are incorporated herein by reference in their entirety. In some embodiments, the microfluidic mixers used are those described in PCT Application No. WO/2014/172045, which is incorporated herein by reference in their entirety.

In some embodiments, the mixing step is performed with a T-junction, confined impinging jets, microfluidic mixer, or vortex mixer.

In some embodiments, the loading step is performed with a T-junction, confined impinging jets, microfluidic mixer, or vortex mixer.

In some embodiments, the mixing step is performed at a temperature of less than about 30° C., less than about 28° C., less than about 26° C., less than about 24° C., less than about 22° C., less than about 20° C., or less than about ambient temperature.

In some embodiments, the loading step is performed at a temperature of less than about 30° C., less than about 28° C., less than about 26° C., less than about 24° C., less than about 22° C., less than about 20° C., or less than about ambient temperature.

In some embodiments, the step of processing the empty-LNP solution or loaded-LNP solution comprises a first adding step, comprising adding a polyethylene glycol lipid (PEG lipid) to the empty LNP or the loaded LNP.

In some embodiments, the step of processing the empty-LNP solution comprises a first adding step, comprising adding a polyethylene glycol lipid (PEG lipid) to the empty LNP solution.

In some embodiments, the step of processing the empty-LNP solution comprises a first adding step, comprising adding a polyethylene glycol lipid (PEG lipid) to the loaded LNP.

In some embodiments, the step of processing the loaded-LNP solution comprises a first adding step, comprising adding a polyethylene glycol lipid (PEG lipid) to the loaded LNP solution.

In some embodiments, the step of processing the loaded-LNP solution comprises a first adding step, comprising adding a polyethylene glycol lipid (PEG lipid) to the empty LNP.

In some embodiments, the first adding step comprises adding a polyethylene glycol solution (PEG solution) comprising the PEG lipid to the empty-LNP solution or loaded-LNP solution.

In some embodiments, the step of processing the empty-LNP solution or loaded-LNP solution comprises a second adding step, comprising adding a polyethylene glycol lipid (PEG lipid) to the empty LNP or the loaded LNP.

In some embodiments, the step of processing the empty-LNP solution comprises a second adding step, comprising adding a polyethylene glycol lipid (PEG lipid) to the empty LNP solution.

In some embodiments, the step of processing the empty-LNP solution comprises a second adding step, comprising adding a polyethylene glycol lipid (PEG lipid) to the loaded LNP.

In some embodiments, the step of processing the loaded-LNP solution comprises a second adding step, comprising adding a polyethylene glycol lipid (PEG lipid) to the loaded LNP solution.

In some embodiments, the step of processing the loaded-LNP solution comprises a second adding step, comprising adding a polyethylene glycol lipid (PEG lipid) to the empty LNP.

In some embodiments, the second adding step comprises adding a polyethylene glycol solution (PEG solution) comprising the PEG lipid to the empty-LNP solution or loaded-LNP solution.

In some embodiments, first adding step comprises adding about 0.1 mol % to about 3.0 mol % PEG, about 0.2 mol % to about 2.5 mol % PEG, about 0.5 mol % to about 2.0 mol % PEG, about 0.75 mol % to about 1.5 mol % PEG, about 1.0 mol % to about 1.25 mol % PEG to the empty LNP or the loaded LNP.

In some embodiments, the first adding step comprises adding about 0.1 mol % to about 3.0 mol % PEG, about 0.2 mol % to about 2.5 mol % PEG, about 0.5 mol % to about 2.0 mol % PEG, about 0.75 mol % to about 1.5 mol % PEG, about 1.0 mol % to about 1.25 mol % PEG to the empty-LNP or The loaded-LNP.

In some embodiments, the first adding step comprises adding about 0.1 mol %, about 0.2 mol %, about 0.3 mol %, about 0.4 mol %, about 0.5 mol %, about 0.6 mol %, about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, or about 3.0 mol % of PEG lipid (e.g., PEG₂ₖ-DMG).

In some embodiments, the first adding step comprises adding about 1.75±0.5 mol %, about 1.75±0.4 mol %, about 1.75±0.3 mol %, about 1.75±0.2 mol %, or about 1.75±0.1 mol % (e.g., about 1.75 mol %) of PEG lipid (e.g., PEG₂ₖ-DMG).

In some embodiments, after the first adding step, the empty LNP solution (e.g., the empty LNP) comprises about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, about 4.0 mol %, about 4.1 mol %, about 4.2 mol %, about 4.3 mol %, about 4.4 mol %, about 4.5 mol %, about 4.6 mol %, about 4.7 mol %, about 4.8 mol %, about 4.9 mol %, or about 5.0 mol % of PEG lipid (e.g., PEG₂ₖ-DMG).

In some embodiments, after the first adding step, the loaded LNP solution (e.g., the loaded LNP) comprises about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, about 4.0 mol %, about 4.1 mol %, about 4.2 mol %, about 4.3 mol %, about 4.4 mol %, about 4.5 mol %, about 4.6 mol %, about 4.7 mol %, about 4.8 mol %, about 4.9 mol %, or about 5.0 mol % of PEG lipid (e.g., PEG₂ₖ-DMG).

In some embodiments, the second adding step comprises adding about 0.1 mol % to about 3.0 mol % PEG, about 0.2 mol % to about 2.5 mol % PEG, about 0.5 mol % to about 2.0 mol % PEG, about 0.75 mol % to about 1.5 mol % PEG, about 1.0 mol % to about 1.25 mol % PEG to the empty LNP or the loaded LNP.

In some embodiments, the second adding step comprises adding about 0.1 mol % to about 3.0 mol % PEG, about 0.2 mol % to about 2.5 mol % PEG, about 0.5 mol % to about 2.0 mol % PEG, about 0.75 mol % to about 1.5 mol % PEG, about 1.0 mol % to about 1.25 mol % PEG to the empty LNP or the loaded LNP.

In some embodiments, the second adding step comprises adding about 0.1 mol %, about 0.2 mol %, about 0.3 mol %, about 0.4 mol %, about 0.5 mol %, about 0.6 mol %, about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, or about 3.0 mol % of PEG lipid (e.g., PEG₂ₖ-DMG).

In some embodiments, the second adding step comprises adding about 1.0±0.5 mol %, about 1.0±0.4 mol %, about 1.0±0.3 mol %, about 1.0±0.2 mol %, or about 1.0±0.1 mol % (e.g., about 1.0 mol %) of PEG lipid (e.g., PEG₂ₖ-DMG).

In some embodiments, the second adding step comprises adding about 1.0 mol % PEG lipid to the empty LNP or the loaded LNP.

In some embodiments, after the second adding step, the empty LNP solution (e.g., the empty LNP) comprises about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, about 4.0 mol %, about 4.1 mol %, about 4.2 mol %, about 4.3 mol %, about 4.4 mol %, about 4.5 mol %, about 4.6 mol %, about 4.7 mol %, about 4.8 mol %, about 4.9 mol %, or about 5.0 mol % of PEG lipid (e.g., PEG₂ₖ-DMG).

In some embodiments, after the second adding step, the loaded LNP solution (e.g., the loaded LNP) comprises about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, about 4.0 mol %, about 4.1 mol %, about 4.2 mol %, about 4.3 mol %, about 4.4 mol %, about 4.5 mol %, about 4.6 mol %, about 4.7 mol %, about 4.8 mol %, about 4.9 mol %, or about 5.0 mol % of PEG lipid (e.g., PEG₂ₖ-DMG).

In some embodiments, the first adding step is performed at a temperature of less than about 30° C., less than about 28° C., less than about 26° C., less than about 24° C., less than about 22° C., less than about 20° C., or less than about ambient temperature.

In some embodiments, the second adding step is performed at a temperature of less than about 30° C., less than about 28° C., less than about 26° C., less than about 24° C., less than about 22° C., less than about 20° C., or less than about ambient temperature.

In some embodiments, the step of processing the empty-LNP solution or loaded-LNP solution further comprises at least one step selected from filtering, pH adjusting, buffer exchanging, diluting, dialyzing, concentrating, freezing, lyophilizing, storing, and packing.

In some embodiments, the step of processing the empty-LNP solution or loaded-LNP solution further comprises pH adjusting.

In some embodiments, the pH adjusting comprises adding a second buffering agent is selected from the group consisting of an acetate buffer, a citrate buffer, a phosphate buffer, and a tris buffer. In some embodiments, the first adding step is performed prior to the pH adjusting.

In some embodiments, the first adding step is performed after the pH adjusting.

In some embodiments, the second adding step is performed prior to the pH adjusting.

In some embodiments, the second adding step is performed after the pH adjusting.

In some embodiments, the step of processing the empty-LNP solution or loaded-LNP solution further comprises filtering.

In some embodiments, the filtering is a tangential flow filtration (TFF).

In some embodiments, the filtering removes an organic solvent (e.g., an alcohol or ethanol) from the LNP solution. In some embodiments, upon removal of the organic solvent (e.g. an alcohol or ethanol), the LNP solution is converted to a solution buffered at a neutral pH, pH 6.5 to 7.8, pH 6.8 to pH 7.5, preferably, pH 7.0 to pH 7.2 (e.g., a phosphate or HEPES buffer). In some embodiments, the LNP solution is converted to a solution buffered at a pH of about 7.0 to pH to about 7.2. In some embodiments, the resulting LNP solution is sterilized before storage or use, e.g., by filtration (e.g., through a 0.1-0.5 μm filter).

In some embodiments, the step of processing the empty-LNP solution or loaded-LNP solution further comprises buffer exchanging.

In some embodiments, the buffer exchanging comprises addition of an aqueous buffer solution comprising a third buffering agent.

In some embodiments, the first adding step is performed prior to the buffer exchanging.

In some embodiments, the first adding step is performed after the buffer exchanging.

In some embodiments, the second adding is performed prior to the buffer exchanging.

In some embodiments, the second adding step is performed after the buffer exchanging.

In some embodiments, the step of processing the empty-LNP solution or loaded-LNP solution further comprises diluting.

In some embodiments, the step of processing the empty-LNP solution or loaded-LNP solution further comprises dialyzing.

In some embodiments, the step of processing the empty-LNP solution or loaded-LNP solution further comprises concentrating.

In some embodiments, the step of processing the empty-LNP solution or loaded-LNP solution further comprises freezing.

In some embodiments, the step of processing the empty-LNP solution or loaded-LNP solution further comprises lyophilizing.

In some embodiments, the lyophilizing comprises freezing the loaded-LNP solution at a temperature from about −100° C. to about 0° C., about −80° C. to about −10° C., about −60° C. to about −20° C., about −50° C. to about −25° C., or about −40° C. to about −30° C.

In some embodiments, the lyophilizing further comprises drying the frozen loaded-LNP solution to form a lyophilized empty LNP or lyophilized loaded LNP.

In some embodiments, the drying is performed at a vacuum ranging from about 50 mTorr to about 150 mTorr.

In some embodiments, the drying is performed at about −35° C. to about −15° C.

In some embodiments, the drying is performed at about room temperature to about 25° C.

In some embodiments, the step of processing the empty-LNP solution or loaded-LNP solution further comprises storing.

In some embodiments, the storing comprises storing the empty LNP or the loaded LNP at a temperature of about −80° C., about −78° C., about −76° C., about −74° C., about −72° C., about −70° C., about −65° C., about −60° C., about −55° C., about −50° C., about −45° C., about −40° C., about −35° C., or about −30° C. for at least 1 day, at least 2 days, at least 1 week, at least 2 weeks, at least 4 weeks, at least 1 month, at least 2 months, at least 3 months, at least 6 months, at least 8 months, or at least 1 year.

In some embodiments, the storing comprises storing the empty LNP or the loaded LNP at a temperature of about −40° C., about −35° C., about −30° C., about −25° C., about −20° C., about −15° C., about −10° C., about −5° C., about 0° C., about 5° C., about 10° C., about 15° C., about 20° C., or about 25° C. for at least 1 day, at least 2 days, at least 1 week, at least 2 weeks, at least 4 weeks, at least 1 month, at least 2 months, at least 3 months, at least 6 months, at least 8 months, or at least 1 year.

In some embodiments, the storing comprises storing the empty LNP or the loaded LNP at a temperature of about −40° C. to about 0° C., from about −35° C. to about −5° C., from about −30° C. to about −10° C., from about −25° C. to about −15° C., from about −22° C. to about −18° C., or from about −21° C. to about −19° C. for at least 1 day, at least 2 days, at least 1 week, at least 2 weeks, at least 4 weeks, at least 1 month, at least 2 months, at least 3 months, at least 6 months, at least 8 months, or at least 1 year.

In some embodiments, the storing comprises storing the empty LNP or the loaded LNP at a temperature of about −20° C. for at least 1 day, at least 2 days, at least 1 week, at least 2 weeks, at least 4 weeks, at least 1 month, at least 2 months, at least 3 months, at least 6 months, at least 8 months, or at least 1 year.

In some embodiments, the step of processing the empty-LNP solution or loaded-LNP solution further comprises packing.

As used herein, “packing” may refer to storing a drug product in its final state or in-process storage of an empty LNP, loaded LNP, or LNP formulation before they are placed into final packaging. Modes of storage and/or packing include, but are not limited to, refrigeration in sterile bags, refrigerated or frozen formulations in vials, lyophilized formulations in vials and syringes, etc.

In some embodiments, the step of processing the empty-LNP solution or loaded-LNP solution comprises: iia) adding a cryoprotectant to the empty-LNP solution or loaded-LNP solution.

In some embodiments, the step of processing the empty-LNP solution or loaded-LNP solution comprises: iib) filtering the empty-LNP solution or loaded-LNP solution.

In some embodiments, the step of processing the empty-LNP solution or loaded-LNP solution comprises:
iia) adding a cryoprotectant to the empty-LNP solution or loaded-LNP solution; and
iic) filtering the empty-LNP solution or loaded-LNP solution.

In some embodiments, the step of processing the empty-LNP solution or loaded-LNP solution comprises one or more of the following steps:
iib) adding a cryoprotectant to the empty-LNP solution or loaded-LNP solution;
iic) lyophilizing the empty-LNP solution or loaded-LNP solution, thereby forming a lyophilized LNP composition;
iid) storing the empty-LNP solution or loaded-LNP solution of the lyophilized LNP composition; and
iie) adding a buffering solution to the empty-LNP solution, loaded-LNP solution or the lyophilized LNP composition, thereby forming the LNP formulation.

In some embodiments, the step of processing the empty-LNP solution comprises: iia) adding a cryoprotectant to the empty-LNP solution.

In some embodiments, the step of processing the empty-LNP solution comprises: iib) filtering the empty-LNP solution.

In some embodiments, the step of processing the empty-LNP solution comprises:
iia) adding a cryoprotectant to the empty-LNP solution; and
iic) filtering the empty-LNP solution.

In some embodiments, the cryoprotectant is added to the empty-LNP solution or loaded-LNP solution prior to the lyophilization. In some embodiments, the cryoprotectant comprises one or more cryoprotective agents, and each of the one or more cryoprotective agents is independently a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG₂ₖ-DMG, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium acetate, sodium chloride, sodium formate, sodium malonate, sodium nitrate, sodium sulfate, or any hydrate thereof), or any combination thereof. In some embodiments, the cryoprotectant comprises sucrose. In some embodiments, the cryoprotectant and/or excipient is sucrose. In some embodiments, the cryoprotectant comprises sodium acetate. In some embodiments, the cryoprotectant and/or excipient is sodium acetate. In some embodiments, the cryoprotectant comprises sucrose and sodium acetate.

In some embodiments, the cryoprotectant comprises a cryoprotective agent present at a concentration from about 10 g/L to about 1000 g/L, from about 25 g/L to about 950 g/L, from about 50 g/L to about 900 g/L, from about 75 g/L to about 850 g/L, from about 100 g/L to about 800 g/L, from about 150 g/L to about 750 g/L, from about 200 g/L to about 700 g/L, from about 250 g/L to about 650 g/L, from about 300 g/L to about 600 g/L, from about 350 g/L to about 550 g/L, from about 400 g/L to about 500 g/L, and from about 450 g/L to about 500 g/L. In some embodiments, the cryoprotectant comprises a cryoprotective agent present at a concentration from about 10 g/L to about 500 g/L, from about 50 g/L to about 450 g/L, from about 100 g/L to about 400 g/L, from about 150 g/L to about 350 g/L, from about 200 g/L to about 300 g/L, and from about 200 g/L to about 250 g/L. In some embodiments, the cryoprotectant comprises a cryoprotective agent present at a concentration of about 10 g/L, about 25 g/L, about 50 g/L, about 75 g/L, about 100 g/L, about 150 g/L, about 200 g/L, about 250 g/L, about 300 g/L, about 300 g/L, about 350 g/L, about 400 g/L, about 450 g/L, about 500 g/L, about 550 g/L, about 600 g/L, about 650 g/L, about 700 g/L, about 750 g/L, about 800 g/L, about 850 g/L, about 900 g/L, about 950 g/L, and about 1000 g/L.

In some embodiments, the cryoprotectant comprises a cryoprotective agent present at a concentration from about 0.1 mM to about 100 mM, from about 0.5 mM to about 90 mM, from about 1 mM to about 80 mM, from about 2 mM to about 70 mM, from about 3 mM to about 60 mM, from about 4 mM to about 50 mM, from about 5 mM to about 40 mM, from about 6 mM to about 30 mM, from about 7 mM to about 25 mM, from about 8 mM to about 20 mM, from about 9 mM to about 15 mM, and from about 10 mM to about 15 mM. In some embodiments, the cryoprotectant comprises a cryoprotective agent present at a concentration from about 0.1 mM to about 10 mM, from about 0.5 mM to about 9 mM, from about 1 mM to about 8 mM, from about 2 mM to about 7 mM, from about 3 mM to about 6 mM, and from about 4 mM to about 5 mM. In some embodiments, the cryoprotectant comprises a cryoprotective agent present at a concentration of about 0.1 mM, about 0.5 mM, about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, and about 100 mM.

In some embodiments, the cryoprotectant comprises sucrose.

In some embodiments, the cryoprotectant comprises an aqueous solution comprising sucrose.

In some embodiments, the cryoprotectant comprises an aqueous solution comprising about 700±300 g/L, 700±200 g/L, 700±100 g/L, 700±90 g/L, 700±80 g/L, 700±70 g/L, 700±60 g/L, 700±50 g/L, 700±40 g/L, 700±30 g/L, 700±20 g/L, 700±10 g/L, 700±9 g/L, 700±8 g/L, 700±7 g/L, 700±6 g/L, 700±5 g/L, 700±4 g/L, 700±3 g/L, 700±2 g/L, or 700±1 g/L of sucrose.

In some embodiments, the cryoprotectant comprises an aqueous solution comprising sodium acetate and sucrose.

In some embodiments, the cryoprotectant comprises an aqueous solution comprising:
(a) about 5±1 mM, about 5±0.9 mM, about 5±0.8 mM, about 5±0.5 mM, about 5±0.6 mM, about 5±0.5 mM, about 5±0.4 mM, about 5±0.3 mM, about 5±0.2 mM, or about 5±0.1 mM of sodium acetate; and
(b) about 700±300 g/L, 700±200 g/L, 700±100 g/L, 700±90 g/L, 700±80 g/L, 700±70 g/L, 700±60 g/L, 700±50 g/L, 700±40 g/L, 700±30 g/L, 700±20 g/L, 700±10 g/L, 700±9 g/L, 700±8 g/L, 700±7 g/L, 700±6 g/L, 700±5 g/L, 700±4 g/L, 700±3 g/L, 700±2 g/L, or 700±1 g/L of sucrose.

In some embodiments, the cryoprotectant comprises an aqueous solution comprising sodium acetate and sucrose, wherein the aqueous solution has a pH value of 5.0±2.0, 5.0±1.5, 5.0±1.0, 5.0±0.9, 5.0±0.8, 5.0±0.7, 5.0±0.6, 5.0±0.5, 5.0±0.4, 5.0±0.3, 5.0±0.2, or 5.0±0.1.

In some embodiments, the cryoprotectant comprises an aqueous solution comprising:
(a) about 5±1 mM, about 5±0.9 mM, about 5±0.8 mM, about 5±0.5 mM, about 5±0.6 mM, about 5±0.5 mM, about 5±0.4 mM, about 5±0.3 mM, about 5±0.2 mM, or about 5±0.1 mM of sodium acetate; and
(b) about 700±300 g/L, 700±200 g/L, 700±100 g/L, 700±90 g/L, 700±80 g/L, 700±70 g/L, 700±60 g/L, 700±50 g/L, 700±40 g/L, 700±30 g/L, 700±20 g/L, 700±10 g/L, 700±9 g/L, 700±8 g/L, 700±7 g/L, 700±6 g/L, 700±5 g/L, 700±4 g/L, 700±3 g/L, 700±2 g/L, or 700±1 g/L of sucrose; and
wherein the aqueous solution has a pH value of 5.0±2.0, 5.0±1.5, 5.0±1.0, 5.0±0.9, 5.0±0.8, 5.0±0.7, 5.0±0.6, 5.0±0.5, 5.0±0.4, 5.0±0.3, 5.0±0.2, or 5.0±0.1.

In some embodiments, the lyophilization is carried out in a suitable glass receptacle (e.g., a 10 mL cylindrical glass vial). In some embodiments, the glass receptacle withstands extreme changes in temperatures between lower than −40° C. and higher than room temperature in short periods of time, and/or be cut in a uniform shape. In some embodiments, the step of lyophilizing comprises freezing the LNP solution at a temperature higher than about −40° C., thereby forming a frozen LNP solution; and drying the frozen LNP solution to form the lyophilized LNP composition. In some embodiments, the step of lyophilizing comprises freezing the LNP solution at a temperature higher than about −40° C. and lower than about −30° C. The freezing step results in a linear decrease in temperature to the final over about 6 minutes, preferably at about 1° C. per minute from 20° C. to −40° C. In some embodiments, the freezing step results in a linear decrease in temperature to the final over about 6 minutes at about 1° C. per minute from 20° C. to −40° C. In some embodiments, sucrose at 12-15% may be used, and the drying step is performed at a vacuum ranging from about 50 mTorr to about 150 mTorr. In some embodiments, sucrose at 12-15% may be used, and the drying step is performed at a vacuum ranging from about 50 mTorr to about 150 mTorr, first at a low temperature ranging from about −35° C. to about −15° C., and then at a higher temperature ranging from room temperature to about 25° C. In some embodiments, sucrose at 12-15% may be used, and the drying step is performed at a vacuum ranging from about 50 mTorr to about 150 mTorr, and the drying step is completed in three to seven days. In some embodiments, sucrose at 12-15% may be used, and the drying step is performed at a vacuum ranging from about 50 mTorr to about 150 mTorr, first at a low temperature ranging from about −35° C. to about −15° C., and then at a higher temperature ranging from room temperature to about 25° C., and the drying step is completed in three to seven days. In some embodiments, the drying step is performed at a vacuum ranging from about 50 mTorr to about 100 mTorr. In some embodiments, the drying step is performed at a vacuum ranging from about 50 mTorr to about 100 mTorr, first at a low temperature ranging from about −15° C. to about 0° C., and then at a higher temperature.

In some embodiments, the empty-LNP solution, loaded-LNP solution, or the lyophilized LNP composition is stored at a pH from about 3.5 to about 8.0, from about 4.0 to about 7.5, from about 4.5 to about 7.0, from about 5.0 to about 6.5, and from about 5.5 to about 6.0. In some embodiments, the empty-LNP solution, loaded-LNP solution, or the lyophilized LNP composition is stored at a pH of about 3.5, about 4.0, about 4.5, about 4.6, about 4.7, about 4.8, about 4.9, about 5.0, about 5.1, about 5.2, about 5.3, about 5.4, about 4.5, about 5.5, about 6.5, about 7.0, about 7.5, and about 8.0.

In some embodiments, the LNP solution, loaded-LNP solution, or the lyophilized LNP composition is stored in a cryoprotectant comprising sucrose and sodium acetate. In some embodiments, the LNP solution, loaded-LNP solution, or the lyophilized LNP composition is stored in a cryoprotectant comprising from about 150 g/L to about 350 g/L sucrose and from about 3 mM to about 6 mM sodium acetate at a pH from about 4.5 to about 7.0. In some embodiments, the LNP solution, loaded-LNP solution, or the lyophilized LNP composition is stored in a cryoprotectant comprising about 200 g/L sucrose and 5 mM sodium acetate at about pH 5.0.

In some embodiments, the empty-LNP solution, loaded-LNP solution, or the lyophilized LNP composition is stored at a temperature of about −80° C., about −78° C., about −76° C., about −74° C., about −72° C., about −70° C., about −65° C., about −60° C., about −55° C., about −50° C., about −45° C., about −40° C., about −35° C., or about −30° C. prior to adding the buffering solution.

In some embodiments, the empty-LNP solution, loaded-LNP solution, or the lyophilized LNP composition is stored at a temperature of about −40° C., about −35° C., about −30° C., about −25° C., about −20° C., about −15° C., about −10° C., about −5° C., about 0° C., about 5° C., about 10° C., about 15° C., about 20° C., or about 25° C. prior to adding the buffering solution.

In some embodiments, the empty-LNP solution, loaded-LNP solution, or the lyophilized LNP composition is stored at a temperature of ranging from about −40° C. to about 0° C., from about −35° C. to about −5° C., from about −30° C. to about −10° C., from about −25° C. to about −15° C., from about −22° C. to about −18° C., or from about −21° C. to about −19° C. prior to adding the buffering solution.

In some embodiments, the empty-LNP solution, loaded-LNP solution, or the lyophilized LNP composition is stored at a temperature of about −20° C. prior to adding the buffering solution.

Certain aspects of the methods are described in PCT Application No. WO/2020/160397 which is incorporated herein by reference in their entirety.

Described herein are also cells comprising a nanoparticle. The cells can be mucosal cells. The cells can be epithelial cells. In some embodiments, the cells are not epithelial cells. The cells can be respiratory epithelial cells. For example, the cells can be nasal cells. The cells can be HeLa cells. Such cells can be contacted with LNPs in vitro or in vivo.

### Nucleic Acid Vaccines

The present disclosure, in some embodiments, provides nanoparticles comprising a nucleic acid vaccine (e.g., mRNA vaccine). Exemplary vaccines feature mRNAs encoding a particular antigen or epitope of interest (or an mRNA or mRNAs encoding antigens of interest). In exemplary aspects, the vaccines feature an mRNA or mRNAs encoding antigen(s) derived from infectious diseases or cancers. In some embodiments, the infectious disease is an infectious respiratory disease (e.g., influenza, coronavirus, parainfluenza, respiratory syncytial virus, rhinovirus, parainfluenza, human metapneumovirus, etc.). In some embodiments, the cancer is related to the respiratory system (e.g., tracheal or bronchial cancer).

In some embodiments, the nucleic acid encodes an antigen. Antigens, as used herein, are proteins capable of inducing an immune response (e.g., causing an immune system to produce antibodies against the antigens). The vaccines of the present disclosure provide a unique advantage over traditional protein-based vaccination approaches in which protein antigens are purified or produced in vitro, e.g., recombinant protein production technologies. The vaccines of the present disclosure feature mRNA encoding the desired antigens, which when introduced into the body, i.e., administered to a mammalian subject (for example a human) in vivo, cause the cells of the body to express the desired antigens. In order to facilitate delivery of the mRNAs of the present disclosure to the cells of the body, the mRNAs are encapsulated in lipid nanoparticles (LNPs), as described herein. Upon delivery and uptake by cells of the body, the mRNAs are translated in the cytosol and protein antigens are generated by the host cell machinery. The protein antigens are presented and elicit an adaptive humoral and cellular immune response. Neutralizing antibodies are directed against the expressed protein antigens and hence the protein antigens are considered relevant target antigens for vaccine development. Herein, use of the term “antigen” encompasses immunogenic proteins and immunogenic fragments (an immunogenic fragment that induces (or is capable of inducing) an immune response), unless otherwise stated. It should be understood that the term “protein” encompasses peptides and the term “antigen” encompasses antigenic fragments.

The antigen may be from an infectious disease. A non-limiting list of infectious diseases includes, but is not limited to, viral infectious diseases such as AIDS (HIV), HIV resulting in mycobacterial infection, AIDS related Cacheixa, AIDS related Cytomegalovirus infection, HIV-associated nephropathy, Lipodystrophy, AID related cryptococcal meningitis, AIDS related neutropaenia, Pneumocysitis jiroveci (Pneumocystis carinii) infections, AID related toxoplasmosis, hepatitis A, B, C, D or E, herpes, herpes zoster (chicken pox), German measles (rubella virus), yellow fever, dengue fever etc. (flavi viruses), flu (influenza viruses), haemorrhagic infectious diseases (Marburg or Ebola viruses), bacterial infectious diseases such as Legionnaires' disease (Legionella), gastric ulcer (Helicobacter), cholera (Vibrio), E. coli infections, staphylococcal infections, salmonella infections or streptococcal infections, tetanus (Clostridium tetani), protozoan infectious diseases (malaria, sleeping sickness, leishmaniasis, toxoplasmosis, i.e. infections caused by plasmodium, trypanosomes, leishmania and toxoplasma), diphtheria, leprosy, measles, pertussis, rabies, tetanus, tuberculosis, typhoid, varicella, diarrheal infections such as Amoebiasis, Clostridium difficile-associated diarrhea (CDAD), Cryptosporidiosis, Giardiasis, Cyclosporiasis and Rotaviral gastroenteritis, encephalitis such as Japanese encephalitis, Wester equine encephalitis and Tick-borne encephalitis (TBE), fungal skin diseases such as candidiasis, onychomycosis, Tinea captis/scal ringworm, Tinea corporis/body ringworm, Tinea cruris/jock itch, sporotrichosis and Tinea pedis/Athlete's foot, Meningitis such as Haemophilus influenza type b (Hib), Meningitis, viral, meningococcal infections and pneumococcal infection, neglected tropical diseases such as Argentine haemorrhagic fever, Leishmaniasis, Nematode/roundworm infections, Ross river virus infection and West Nile virus (WNV) disease, Non-HIV STDs such as Trichomoniasis, Human papillomavirus (HPV) infections, sexually transmitted chlamydial diseases, Chancroid and Syphilis, Non-septic bacterial infections such as cellulitis, lyme disease, MRSA infection, pseudomonas, staphylococcal infections, Boutonneuse fever, Leptospirosis, Rheumatic fever, Botulism, Rickettsial disease and Mastoiditis, parasitic infections such as Cysticercosis, Echinococcosis, Trematode/Fluke infections, Trichinellosis, Babesiosis, Hypodermyiasis, Diphyllobothriasis and Trypanosomiasis, respiratory infections such as adenovirus infection, aspergillosis infections, avian (H5N1) influenza, influenza, RSV infections, severe acute respiratory syndrome (SARS), sinusitis, Legionellosis, Coccidioidomycosis and swine (H1N1) influenza, sepsis such as bacteraemia, sepsis/septic shock, sepsis in premature infants, urinary tract infection such as vaginal infections (bacterial), vaginal infections (fungal) and gonococcal infection, viral skin diseases such as B19 parvovirus infections, warts, genital herpes, orofacial herpes, shingles, inner ear infections, fetal cytomegalovirus syndrome, foodborn illnesses such as brucellosis (Brucella species), Clostridium perfringens (Epsilon toxin), E. Coli O157:H7 (Escherichia coli), Salmonellosis (Salmonella species), Shingellosis (Shingella), Vibriosis and Listeriosis, bioterrorism and potential epidemic diseases such as Ebola haemorrhagic fever, Lassa fever, Marburg haemorrhagic fever, plague, Anthrax Nipah virus disease, Hanta virus, Smallpox, Glanders (Burkholderia mallei), Melioidosis (Burkholderia pseudomallei), Psittacosis (Chlamydia psittaci), Q fever (Coxiella burnetii), Tularemia (Fancisella tularensis), rubella, mumps and polio.

In some embodiments, the antigen is from a respiratory infectious disease. Examples of respiratory infectious diseases include tuberculosis, pertussis, influenza, coronavirus (e.g., SARS, MERS), diphtheria, streptococcus, Legionnaires' disease, measles, mumps, pneumonia, pneumococcal menigitis, rubella, and tuberculosis. In some embodiments, the vaccine is a coronavirus vaccine (e.g., a SARS-CoV-2 vaccine). In some embodiments, the vaccine is an influenza vaccine. In some embodiments, the vaccine is a parainfluenza vaccine (e.g., PIV3 vaccine). In some embodiments, the vaccine is a respiratory syncytial virus (RSV) vaccine. In some embodiments, the vaccine is a human metapneumovirus (hMPV) vaccine. In some embodiements, the vaccine comprises a combination of antigens from a single virus (e.g., is multivalent) or from multiple viruses (e.g., is a combination vaccine). For example, the vaccine may be a coronavirus (e.g., SARS-CoV-2) and flu vaccine; a coronavirus (e.g., SARS-CoV-2), flu, and RSV vaccine; an PIV3 and hMPV vaccine; an RSV, PIV3, and hMPV vaccine; or any combination of the vaccines provided herein.

In some embodiments, the vaccine is a CMV vaccine.

In some embodiments, the vaccine is a cancer vaccine, and the nucleic acids encode one or more cancer antigens. In some embodiments, the one or more cancer antigens are specific to the subject (that is, the vaccine is a personalized cancer vaccine). In some embodiments, the one or more cancer antigens are shared cancer antigens (also called traditional cancer antigens). Cancer antigens, or tumor-associated antigens are antigens that are expressed in or by tumor cells. A particular tumor associated antigen may or may not also be expressed in non-cancerous cells. Many tumor mutations are well known in the art. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes are completely foreign to the body and thus would not produce an immune response against healthy tissue or be masked by the protective components of the immune system. In some embodiments personalized vaccines based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and/or translocations.

Examples of tumor-associated antigens include, but are not limited to, 5 alpha reductase, alpha-fetoprotein, AM-1, APC, April, BAGE, beta-catenin, Bcl12, bcr-abl, CA-125, CASP-8/FLICE, Cathepsins, CD19, CD20, CD21, CD23, CD22, CD33 CD35, CD44, CD45, CD46, CD5, CD52, CD55, CD59, CDC27, CDK4, CEA, c-myc, Cox-2, DCC, DcR3, E6/E7, CGFR, EMBP, Dna78, farnesyl transferase, FGF8b, FGF8a, FLK-1/KDR, folic acid receptor, G250, GAGE-family, gastrin 17, gastrin-releasing hormone, GD2/GD3/GM2, GnRH, GnTV, GP1, gp100/Pme117, gp-100-in4, gp15, gp75/TRP-1, hCG, heparanse, Her2/neu, HMTV, Hsp70, hTERT, IGFR1, IL-13R, iNOS, Ki67, KIAA0205, K-ras, H-ras, N-ras, KSA, LKLR-FUT, MAGE-family, mammaglobin, MAP17, melan-A/MART-1, mesothelin, MIC A/B, MT-MMPs, mucin, NY-ESO-1, osteonectin, p15, P170/MDR1, p53, p97/melanotransferrin, PAI-1, PDGF, uPA, PRAME, probasin, progenipoientin, PSA, PSM, RAGE-1, Rb, RCAS1, SART-1, SSX-family, STAT3, STn, TAG-72, TGF-alpha, TGF-beta, Thymosin-beta-15, TNF-alpha, TRP-1, TRP-2, tyrosinase, VEGF, ZAG, p16INK4, and glutathione-S-transferase.

The nucleic acid vaccines of the present disclosure, in some embodiments, comprise a (at least one) messenger RNA (mRNA) having an open reading frame (ORF) encoding an influenza virus antigen. In some embodiments, the mRNA further comprises a 5′ UTR, 3?UTR, a poly(A) tail and/or a 5′ cap analog.

It should also be understood that the vaccines of the present disclosure may include any 5′ untranslated region (UTR) and/or any 3′ UTR. Exemplary UTR sequences include SEQ ID NOs: 1-4; however, other UTR sequences may be used or exchanged for any of the UTR sequences described herein. In some embodiments, a 5′ UTR of the present disclosure comprises a sequence selected from

SEQ ID NO: 1
(GGGAAAUAAGAGAGAAAAGAAGAGUAAGAAGAAAUAUAAGAGCCACC)
and
SEQ ID NO: 2
(GGGAAAUAAGAGAGAAAAGAAGAGUAAGAAGAAAUAUAAGACCCCGGC
GCCGCCACC).

In some embodiments, a 3′ UTR of the present disclosure comprises a sequence selected from SEQ ID NO: 3 (UGAUAAUAGGCUGGAGCCUCGGUGGCCAUGCUUCUUGCCCCUUGGGCCUCCCCCCAGCCC CUCCUCCCCUUCCUGCACCCGUACCCCCGUGGUCUUUGAAUAAAGUCUGAGUGGGCGGC) and SEQ ID NO: 4 (UGAUAAUAGGCUGGAGC CUCGGUGGCCUAGCUUCUUGCCCCUUGGGCCUCCCCCCAGCCCCUCCUCCCCUUCCUGCAC CCGUACCCCCGUGGUCUUUGAAUAAAGUCUGAGUGGGCGGC). UTRs may also be omitted from the RNA polynucleotides provided herein.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents mRNA, the “T”s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

In some embodiments, the nucleic acids of the vaccines comprise one or more stabilizing agents. Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition comprises an mRNA having an ORF that encodes a signal peptide fused to the virus antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, an mRNA is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

Pharmaceutical Compositions and Formulations The present disclosure provides pharmaceutical compositions and formulations that comprise any of nanoparticles described herein and polynucleotide or polypeptide payload vaccines (e.g., mRNA vaccines or therapeutics).

Pharmaceutical compositions or formulations can optionally comprise one or more additional active substances, e.g., therapeutically and/or prophylactically active substances. The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Pharmaceutical compositions or formulations of the present disclosure can be sterile and/or pyrogen-free. General considerations in the formulation and/or manufacture of pharmaceutical agents can be found, for example, in Remington: The Science and Practice of Pharmacy 21^{st} ed., Lippincott Williams & Wilkins, 2005 (incorporated herein by reference in its entirety). In some embodiments, compositions are administered to humans, human patients or subjects. For the purposes of the present disclosure, the phrase “active ingredient” generally refers to the nanoparticle comprising the payload to be delivered as described herein.

Formulations and pharmaceutical compositions described herein can be prepared by any method known or hereafter developed in the art of pharmacology. In general, such preparatory methods include the step of associating the nanoparticle with an excipient and/or one or more other accessory ingredients, and then, if necessary and/or desirable, dividing, shaping and/or packaging the product into a desired single- or multi-dose unit.

A pharmaceutical composition or formulation in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered.

Although the descriptions of pharmaceutical compositions and formulations provided herein are principally directed to pharmaceutical compositions and formulations that are suitable for administration to humans, it will be understood by the skilled artisan that such compositions are generally suitable for administration to any other animal, e.g., to non-human animals, e.g. non-human mammals.

A pharmaceutically acceptable excipient, as used herein, includes, but is not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, MD, 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary granulating and/or dispersing agents include, but are not limited to, starches, pregelatinized starches, or microcrystalline starch, alginic acid, guar gum, agar, poly(vinyl-pyrrolidone), (providone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate (VEEGUM®), sodium lauryl sulfate, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN@80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ@30]), PLUORINC®F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Oxidation is a potential degradation pathway for mRNA, especially for liquid mRNA formulations. In order to prevent oxidation, antioxidants can be added to the formulations. Exemplary antioxidants include, but are not limited to, alpha tocopherol, ascorbic acid, acorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of polynucleotide solutions are maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition described here can contain a bulking agent in lyophilized polynucleotide formulations to yield a “pharmaceutically elegant” cake, stabilize the lyophilized polynucleotides during long term (e.g., 36 month) storage. Exemplary bulking agents of the present disclosure can include, but are not limited to sucrose, trehalose, mannitol, glycine, lactose, raffinose, and combinations thereof.

The compositions can be in a liquid form or a solid form. In some embodiments, the compositions or formulations are in a liquid form. In some embodiments, the compositions are suitable for inhalation.

In some embodiments, the compositions are administered to the mucosa (e.g., mucosal surface or mucosal membrane). The term “mucosa” refers to an internal wall of, particularly, a hollow organ which communicates with the outside, such as the digestive organ, the respiratory organ, the urogenital organ, or the eye, in vertebrates. As used herein, “mucosal administration” refers to the introduction of any one of the compositions described herein into the body via any mucosal surface, such as intragastrically, pulmonarily, transdermally, intestinally, ocularly, intranasally, orally, vaginally, or rectally.

The compositions can be administered to the respiratory tract. Aerosolized pharmaceutical formulations can be delivered to the nasal passages, preferably using a number of commercially available devices.

Compositions can be administered to the respiratory tract by suitable methods such as intranasal instillation, intratracheal instillation, and intratracheal injection. In some embodiments, the compositions or the nanoparticle is administered by intranasal, or intrabronchial administration. In some embodiments, the compositions or the nanoparticle is administered via intranasal administration. Intranasal administration, in some embodiments, refers to administration of a dosage form formulated and delivered topically to the nasal epithelium. For example, the compositions and nanoparticles are administered by nebulizer or inhaler or droplet administration to a nasal surface.

In some embodiments, the compositions are delivered into the respiratory system (e.g., nose and/or trachea) by inhalation of an aerosolized pharmaceutical formulation. Inhalation can occur through the nose and/or the mouth of the subject. In some embodiments, inhalation occurs through the nose (e.g., a liquid solution or droplet or dry powder is inhaled through the nose). Administration can occur by self-administration of the formulation while inhaling, or by administration of the formulation via a respirator to a subject on a respirator. Exemplary devices for delivering formulations to the respiratory system (e.g., nose and/or trachea) include, but are not limited to, dry powder inhalers, pressurized metered dose inhalers, nebulizers, and electrohydrodynamic aerosol devices.

Liquid formulations can be administered to the respiratory system (e.g., nose and/or trachea) of a patient using a pressurized metered dose inhaler (pMDI). pMDIs generally include at least two components: a canister in which the liquid formulation is held under pressure in combination with one or more propellants, and a receptacle used to hold and actuate the canister. The canister may contain a single or multiple doses of the formulation. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the liquid formulation is atomized, forming an aerosol. pMDIs typically employ one or more propellants to pressurize the contents of the canister and to propel the liquid formulation out of the receptacle outlet, forming an aerosol. Any suitable propellants may be utilized. The propellant may take a variety of forms. For example, the propellant may be a compressed gas or a liquefied gas.

The liquid formulations can also be administered using a nebulizer. Nebulizers are liquid aerosol generators that convert the liquid formulation into mists or clouds of small droplets, preferably having diameters less than 5 microns mass median aerodynamic diameter, which can be inhaled into the lower respiratory tract. This process is called atomization. The droplets carry the one or more active agents into the nose or upper airways when the aerosol cloud is inhaled. Any type of nebulizer may be used to administer the formulation to a patient, including, but not limited to pneumatic (jet) nebulizers and electromechanical nebulizers. Pneumatic (jet) nebulizers use a pressurized gas supply as a driving force for atomization of the liquid formulation. Compressed gas is delivered through a nozzle or jet to create a low pressure field which entrains a surrounding liquid formulation and shears it into a thin film or filaments. The film or filaments are unstable and break up into small droplets that are carried by the compressed gas flow into the inspiratory breath. Baffles inserted into the droplet plume screen out the larger droplets and return them to the bulk liquid reservoir. Electromechanical nebulizers use electrically generated mechanical force to atomize liquid formulations. The electromechanical driving force can be applied, for example, by vibrating the liquid formulation at ultrasonic frequencies, or by forcing the bulk liquid through small holes in a thin film. The forces generate thin liquid films or filament streams which break up into small droplets to form a slow moving aerosol stream which can be entrained in an inspiratory flow. Liquid formulations can also be administered using an electrohydrodynamic (EHD) aerosol device. EHD aerosol devices use electrical energy to aerosolize liquid drug solutions or suspensions.

Dry powder inhalers (DPIs) typically use a mechanism such as a burst of gas to create a cloud of dry powder inside a container, which can then be inhaled by the subject. In a DPI, the dose to be administered is stored in the form of a non-pressurized dry powder and, on actuation of the inhaler, the particles of the powder are inhaled by the subject. In some cases, a compressed gas (i.e., propellant) may be used to dispense the powder, similar to pressurized metered dose inhalers (pMDIs). In some cases, the DPI may be breath actuated, meaning that an aerosol is created in precise response to inspiration. Typically, dry powder inhalers administer a dose of less than a few tens of milligrams per inhalation to avoid provocation of cough. Examples of DPIs include the Turbohaler® inhaler (Astrazeneca, Wilmington, Del.), the Clickhaler® inhaler (Innovata, Ruddington, Nottingham, UKL), the Diskus® inhaler (Glaxo, Greenford, Middlesex, UK), the EasyHaler® (Orion, Expoo, FI), the Exubera® inhaler (Pfizer, New York, N.Y.), the Qdose® inhaler (Microdose, Monmouth Junction, N.J.), and the Spiros® inhaler (Dura, San Diego, Calif.).

In some embodiments, the compositions are administered to the mucosa (e.g., mucosal surface or mucosal membrane). As used herein, “mucosal administration” refers to the introduction of any one of the compositions described herein into the body via any mucosal surface, such as sublingually, intragastrically, buccally, intestinally, ocularly, intranasally, orally, vaginally, or rectally. As used herein, the term “sublingual administration” means absorption of a compound or a pharmaceutically acceptable formulation of a compound by administering under the tongue. “Intragastric administration” refers to the administration of any one of the formulations described herein directly to a subject's stomach (e.g., via gastric tube). Intestinal administration refers to the administration of any one of the formulations described herein directly to a subject's intestine (e.g., small intestine). In some embodiments the administration is not pulmonary administration. In some embodiments the compositions are not administered to the lung epithelial cells.

In some embodiments, the formulations are administered buccally. Buccal administration is administration by absorption into the gum, into the cheek, or both. Sublingual administration is by placement of the dosage form under the tongue. Buccal and sublingual administration are typically accomplished using a solid oral dosage form, or gel. As a non-limiting example, buccal and/or sublingual administration may be used for administration of microorganisms from the mouth of a donor.

In some embodiments, the formulations provided herein are administered orally. Oral administration is administration into the mouth or administration into the mouth with swallowing. Oral administration includes, without limitation, the administration of solid oral dosage forms, liquid dosage forms, gels, pastes, sprays, or any combination thereof. Solid oral dosage forms include, without limitation, capsules, both hard shell and soft shell, tablets, pills, powders, and granules. Liquid dosage forms for oral administration include, without limitation, emulsions, solutions, suspensions, syrups and elixirs. Granules or powders may be reconstituted as an oral suspension or solution for administration.

In some embodiments, the formulations provided herein are administered ocularly. As used herein, “ocular administration” refers to the application of the compositions described herein to the eye of the subject (e.g., the mucous membranes around the eye, such as the conjunctiva).

In some embodiments, the formulations provided herein are delivered intravaginally. As used herein, “intravaginal administration” refers to a mode of administration wherein the compositions or formulations are administered via the vagina so that the formulations are locally absorbed by the vaginal mucosa. Intravaginal administration provides for rapid delivery of the agents to localized areas and tissues such that therapeutically effective drug concentrations are achieved locally, in the region of the diseased or otherwise abnormal tissue, i.e., the tissues or organs in proximity to the vagina, such as the uterus. In some embodiments, the compositions provided herein comprise one or more pharmaceutically acceptable carriers and/or excipients suitable for incorporation into a formulation or delivery system for intravaginal administration, and selected according to the particular type of formulation, i.e., gel, ointment, vaginal suppository, or others. In general, these auxiliary agents are physiologically acceptable and may be naturally occurring or may be of synthetic origin. Ideally, the carriers and/or excipients will be gradually broken down into innocuous substances in the body, or are of a nature that allows them to be secreted by the vagina and washed cleanly from the skin. In either case, they do not clog pores in skin or mucous membranes, leave any unacceptable residues, or cause other adverse effects. In some embodiments, the pharmaceutical compositions comprise liquid carriers (e.g., water or saline), preservatives, thickening agents, lubricating agents, permeation enhancers, emulsifying agents, pH buffering agents, disintegrating agents, binders, coloring agents, viscosity controlling agents, and the like. Mucoadhesive agents such as hydroxypropyl methylcellulose (HPMC) for facilitating prolonged contact with the vaginal wall are also exemplary excipients.

In some embodiments, the formulations provided herein are delivered rectally. Rectal administration refers to a type of administration of a therapeutic agent, wherein the formulation is administered into the rectum. In some embodiments, the compositions described herein are formulation for rectal delivery, which encompasses pharmaceutical formulations that are suitable for the rectum such as a suppository. In some embodiments, the composition is provided as an enema.

The pharmaceutical compositions of the invention are administered in an effective effective amount to cause a desired biological effect, e.g., a prophylactic effect, e.g., owing to expression of an antigen. The formulations may be administered in an effective amount to deliver LNP to, e.g., the apical membrane of respiratory and non-respiratory epithelial cells to deliver a polynucleotide (e.g., polynucleotide encoding an antigen). In some embodiments, the pharmaceutical compositions are administered in an effective amount to induce an immune response sufficient to provide an induced or boosted immune response as a function of antigen production in the cells of the subject. An “effective amount” of a composition (e.g., comprising RNA) is based, at least in part, on the target tissue, target cell type, means of administration, physical characteristics of the payload such as RNA (e.g., length, nucleotide composition, and/or extent of modified nucleosides), other components of the composition, and other determinants, such as age, body weight, height, sex and general health of the subject. The effective amount of the RNA, as provided herein, may be as low as 50 μg (total mRNA), administered for example as a single dose or as two 25 μg doses. A “dose” as used herein, represents the sum total of RNA in the composition (e.g., including all of the antigens in the formulation). In some embodiments, the effective amount is a total dose of 50 μg-300 μg, 100 μg-300 μg, 150 μg-300 μg, 200 μg-300 μg, 250 μg-300 μg, 150 μg-200 μg, 150 μg-250 μg, 150 μg-300 μg, 200 μg-250 μg, or 250 μg-300 μg. For example, the effective amount may be a total dose of 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, or 300 μg.

### Methods of Use

Described herein are methods of treating or preventing a disease in a patient. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, or 6 months. In exemplary embodiments, the time of administration between the initial administration of the prophylactic composition and the booster may be, but is not limited to, 1 week, 2 weeks, 3 weeks, 1 month, 2 months, 3 months, or 6 months. In some embodiments, the time of administration between the initial administration of the prophylactic composition and the booster is 21 days. In some embodiments, the time of administration between the initial administration of the prophylactic composition and the booster is 28 days. In some embodiments, the time of administration between the initial administration of the prophylactic composition and the booster is 36 days. In some embodiments, the time of administration between the initial administration of the prophylactic composition and the booster is 5 months. In some embodiments, the time of administration between the initial administration of the prophylactic composition and the booster is 6 months.

In some embodiments, a composition may be administered intramuscularly, intranasally or intradermally, similarly to the administration of inactivated vaccines known in the art. In some embodiments, the administration schedule is heterologous: for example, a first composition is administered intranasally, and a booster composition is administered via a different route (e.g., intramuscularly). In some embodiments, the first composition is administered intramuscularly, and the booster composition is administered intranasally. In some embodiments, a “prime and pull” vaccination strategy is employed. That is, in some embodiments, a first vaccine (“prime”) is administered intramuscularly to elicit systemic T-cell responses and a second vaccine (booster, “pull”) is administered intranasally to recruit activated T-cells (for example, to a site of infection). In some embodiments, the prime and booster combination is synergistic—that is, the vaccination strategy elicits a stronger and/or more durable immune response than that of each component administered alone.

A composition may be utilized in various settings depending on the prevalence of the disease or disorder, for instance an infection or the degree or level of unmet medical need. As a non-limiting example, an RNA vaccine may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including RNA and/or complexes optionally in combination with one or more pharmaceutically acceptable excipients.

The RNA may be formulated or administered alone or in conjunction with one or more other components. For example, an immunizing composition may comprise other components including, but not limited to, adjuvants. In some embodiments, an immunizing composition does not include an adjuvant (they are adjuvant free).

An RNA may be formulated or administered in combination with one or more pharmaceutically-acceptable excipients. In some embodiments, compositions comprise at least one additional active substances, such as, for example, a therapeutically-active substance, a prophylactically-active substance, or a combination of both. Compositions may be sterile, pyrogen-free or both sterile and pyrogen-free. General considerations in the formulation and/or manufacture of pharmaceutical agents, such as compositions, may be found, for example, in Remington: The Science and Practice of Pharmacy 21st ed., Lippincott Williams & Wilkins, 2005 (incorporated herein by reference in its entirety).

In some embodiments, an immunizing composition is administered to humans, human patients or subjects. In some embodiments, the subject is a human subject under the age of one year (e.g., 1 month, 2 months, 3 months, 4 months, 5 months, 6 months, 7 months, 8 months, 9 months, 10 months, or 11 months of age). In other embodiments, the subject is 1, 2, 3, 4, 5, 6, 7, 8, 9, 10, 11, 12, 13, 14, 15, 16, 17, or 18 years of age. In other embodiments, the subject is 20-25 years of age, 25-30 years of age, 30-35 years of age, 40-45 years of age, 45-50 years of age, 50-60 years of age, 60-70 years of age, 70-80 years of age, 80-90 years of age, 90-100 years of age, or older. For the purposes of the present disclosure, the phrase “active ingredient” generally refers to the RNA or the polynucleotides contained therein, for example, RNA polynucleotides (e.g., mRNA polynucleotides) encoding antigens or therapeutics.

In some embodiments, the mucosal (e.g., intranasal) administration of any one of the compositions provided herein results in the systemic delivery of the composition. As used herein, “systemic delivery” refers to the delivery of a therapeutic product that can result in a broad exposure of an active agent within a subject (e.g., through the circulation). As the nasal mucosa is vascularized, most compositions will be absorbed through the mucosa and into the subject's circulatory system for systemic administration. In this way, mucosal administration bypasses some of the difficulties associated with other types of administration. With respect to vaccines, it is noted that the nasal mucosa is frequently exposed to dust and microbes and is therefore immune competent. Due to the presence of nasal-associated lymphoid tissue (NALT) in the nasal mucosa, intranasal vaccines, in some embodiments, may result in mucosal protection (at the site of infection) in addition to systemic protection (antibody formation and activation of circulating immune cells). In some embodiments, the systemic delivery is a therapeutic effective amount of a polynucleotide or polypeptide payload.

In some embodiments, the disclosure provides for the mucosal (e.g., intranasal) delivery of a payload (e.g., mRNA encoding a therapeutic protein) to the central nervous system (CNS). Delivery to the CNS is complicated due to blood-brain barrier (BBB), a network of endothelial cells coupled by tight junctions that govern solution flow and movement of compounds in and out of the brain parenchyma and that consequently reduces the effective concentration of a systemically administered compound able to reach the brain. Existing methods for delivering therapeutics including systemic administration and precises surgical injections. Certain small molecule, peptide, and protein therapeutics given systemically may reach the brain parenchyma by crossing the BBB; however, high systemic doses are needed to achieve therapeutic levels. High systemic doses may, in some instances, lead to adverse effects. Alternatively, therapeutics may be introduced directly into the CNS with intracerebroventricular or intraparenchymal injections, but these delivery methods are invasive and risky, requiring surgical expertise. In addition, the injections may result in inadequate CNS exposure due to slow diffusion from the injection site and rapid turnover of the cerebrospinal fluid (CSF). Without wishing to be bound by theory, it is thought that mucosal administration of the compositions described herein bypasses the BBB and rapidly targets payload molecules directly to the CNS using pathways along the olfactory and trigeminal nerves innervating the nasal passages.

Therefore, in some embodiments, the present disclosure provides methods of treating or preventing diseases or disorders of the CNS. CNS disorders include genetic disorders, neurodegenerative disorders, psychiatric disorders, and tumors. Exemplary CNS disorders include, but are not limited to, Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder, mania, manic psychosis,), schizophrenia, schizoaffective disorder, schizophreniform disorder, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions, psychosis not otherwise specified (Psychosis NOS),), dementia, aging, paranoia, attention deficit disorder, psychosexual disorders, sleeping disorders, pain disorders, eating or weight disorders (e.g., obesity, cachexia, anorexia nervosa, and bulimia), ophthalmic disorders involving the retina, posterior tract, and optic nerve (e.g., retinitis pigmentosa, diabetic retinopathy and other retinal degenerative diseases, uveitis, age-related macular degeneration, glaucoma), and cancers and tumors (e.g., pituitary tumors) of the CNS.

Formulations of the compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Kits and Devices

The present disclosure provides a variety of kits for conveniently and/or effectively using the claimed nanoparticles of the present disclosure. Typically, kits will comprise sufficient amounts and/or numbers of components to allow a user to perform multiple treatments of a subject(s) and/or to perform multiple experiments.

In one aspect, the present disclosure provides kits comprising the nanoparticles of the present disclosure.

The kit can further comprise packaging and instructions and/or a delivery agent to form a formulation composition. The delivery agent can comprise a saline, a buffered solution, a lipidoid or any delivery agent disclosed herein. In one embodiment, such a kit further comprises an administration device such as a nebulizer or an inhaler.

### Respiratory Function and Other Tests for Improvement in Respiratory Symptoms

In some embodiments, a nanoparticle or pharmaceutical composition comprising an mRNA comprising an open reading frame (ORF) encoding a polypeptide or protein, such as an antigen. Such a polypeptide or protein can be tested for improvement to respiratory function or symptoms (e.g., after exposure to a virus). Respiratory volumes are the amount of air inhaled, exhaled and stored within the lungs at any given time. Non-limiting examples of various respiratory volumes that may be measured are provided below.

Total lung capacity (TLC) is the volume in the lungs at maximal inflation, the sum of VC and RV. The average total lung capacity is 6000 ml, although this varies with age, height, sex and health.

Tidal volume (TV) is the volume of air moved into or out of the lungs during quiet breathing (TV indicates a subdivision of the lung; when tidal volume is precisely measured, as in gas exchange calculation, the symbol TV or VT is used). The average tidal volume is 500 ml.

Residual volume (RV) is the volume of air remaining in the lungs after a maximal exhalation. Residual volume (RV/TLC %) is expressed as percent of TLC.

Expiratory reserve volume (ERV) is the maximal volume of air that can be exhaled (above tidal volume) during a forceful breath out.

Inspiratory reserve volume (IRV) is the maximal volume that can be inhaled from the end-inspiratory position.

Inspiratory capacity (IC) is the sum of IRV and TV.

Inspiratory vital capacity (IVC) is the maximum volume of air inhaled from the point of maximum expiration.

Vital capacity (VC) is the volume of air breathed out after the deepest inhalation.

Functional residual capacity (FRC) is the volume in the lungs at the end-expiratory position.

Forced vital capacity (FVC) is the determination of the vital capacity from a maximally forced expiratory effort.

Forced expiratory volume (time) (FEVₜ) is a generic term indicating the volume of air exhaled under forced conditions in the first t seconds. FEV₁ is the volume that has been exhaled at the end of the first second of forced expiration. FEFₓ is the forced expiratory flow related to some portion of the FVC curve; modifiers refer to amount of FVC already exhaled. FEFₘₐₓ is the maximum instantaneous flow achieved during a FVC maneuver.

Forced inspiratory flow (FIF) is a specific measurement of the forced inspiratory curve, denoted by nomenclature analogous to that for the forced expiratory curve. For example, maximum inspiratory flow is denoted FIFₘₐₓ. Unless otherwise specified, volume qualifiers indicate the volume inspired from RV at the point of measurement.

Peak expiratory flow (PEF) is the highest forced expiratory flow measured with a peak flow meter.

Maximal voluntary ventilation (MVV) is the volume of air expired in a specified period during repetitive maximal effort.

### Process of Preparing LNPs

The present disclosure also provides a process of preparing a lipid nanoparticle composition comprising combining the lipid amine compound disclosed herein, or a salt thereof, with one or more additional lipids selected from:
(i) an ionizable lipid,
(ii) a phospholipid,
(iii) a structural lipid, and
(iv) a PEG-lipid.

In some embodiments, a process of preparing a lipid nanoparticle composition comprises:
(a) mixing a nucleic acid payload with a lipid solution comprising:
   (1) an ionizable lipid,
   (2) a phospholipid,
   (3) a structural lipid, and
   (4) optionally a PEG-lipid
      resulting in a filled lipid nanoparticle (fLNP) core; and
(c) contacting the fLNP core with the lipid amine.

In some embodiments, a process of preparing a nanoparticle comprises:
(a) mixing a lipid solution comprising:
   (1) an ionizable lipid,
   (2) a phospholipid,
   (3) a structural lipid, and
   (4) optionally a PEG-lipid
      resulting in an empty lipid nanoparticle (eLNP) core;
(b) contacting the eLNP core with a nucleic acid payload forming an fLNP; and
(c) contacting the fLNP core with the lipid amine.

In some embodiments, the combining comprises nanoprecipitation. Nanoprecipitation is the unit operation in which the nanoparticles are self-assembled from their individual lipid components by way of kinetic mixing and subsequent maturation and continuous dilution.

In some embodiments, the present disclosure provides a process for preparing a lipid nanoparticle composition comprising:
(1) mixing of an aqueous input and an organic input,
(2) optionally allowing for maturation of the resulting lipid nanoparticle composition, and
(3) optionally diluting the resulting lipid nanoparticle composition.

In some embodiments, the process includes the continuous inline combination of more than 1 (e.g., three) liquid streams with one inline maturation step.

In some embodiments, the organic input comprises a lipid amine compound disclosed herein (e.g., Formula A1) and one or more additional lipids. In some embodiments, the organic input comprises a lipid amine compound disclosed herein, an ionizable lipid, a phospholipid, a structural lipid, and optionally a PEG-lipid. In some embodiments, the organic input comprises a lipid amine compound disclosed herein, an ionizable lipid, a phospholipid, and a structural lipid.

In some embodiments, the organic input comprises a lipid amine and one or more additional lipids dissolved in an organic solvent. In some embodiments, the organic solvent is dimethylsulfoxide, acetone, acetonitrile, ethylene glycol, 1,4-dioxane, 1,3-butanediol, 2-butoxyethanol, or dimethylformamide. In some embodiments, the organic solvent is an organic alcohol. In some embodiments, the organic alcohol is a C₁₋₁₀ hydroxyalkyl. In some embodiments, the organic alcohol is methanol, ethanol, or isopropanol. In some embodiments, the organic alcohol is ethanol.

In some embodiments, the organic input has a lipid concentration of about 1 to about 50 mM, about 5 to about 35 mM, about 10 to about 20 mM, or about 12.5 mM.

In some embodiments, the organic input comprises about 20 mol % to about 50 mol %, about 25 mol % to about 45 mol %, or about 30 mol % to about 40 mol % of ionizable lipid with respect to total lipids. In some embodiments, the organic input comprises about 5 mol % to about 20 mol %, about 8 mol % to about 15 mol %, or about 10 mol % to about 12 mol % of phospholipid with respect to total lipids. In some embodiments, the organic input comprises about 30 mol % to about 50 mol %, about 35 mol % to about 45 mol %, or about 37 mol % to about 42 mol % of structural lipid with respect to total lipids. In some embodiments, the organic input comprises about 0.1 mol % to about 5 mol %, about 0.5 mol % to about 2.5 mol %, or about 1 mol % to about 2 mol % of PEG-lipid with respect to total lipids. In some embodiments, the organic input comprises about 5 mol % to about 30 mol %, about 10 mol % to about 25 mol %, or about 12 mol % to about 20 mol % of lipid amine with respect to total lipids.

In some embodiments, the lipid solution comprises:
about 30 mol % to about 40 mol % of ionizable lipid;
about 10 mol % to about 12 mol % of phospholipid;
about 37 mol % to about 42 mol % of structural lipid;
about 1 mol % to about 2 mol % of PEG-lipid; and
about 12 mol % to about 20 mol % of lipid amine; each with respect to total lipids.

In some embodiments, the lipid solution comprises:
about 33 mol % of ionizable lipid;
about 11 mol % to about 12 mol % of phospholipid;
about 39.5 mol % of structural lipid;
about 1.5 mol % of PEG-lipid; and
about 15 mol % lipid amine; each with respect to total lipids.

In some embodiments, the aqueous input comprises water. In some embodiments, the aqueous input comprises an aqueous buffer solution. In some embodiments, the aqueous buffer solution has a pH of about 3.5 to about 4.5. In further embodiments, the aqueous buffer solution has a pH of about 4. In some embodiments, the aqueous buffer solution has a pH of about 4.6 to about 6.5. In some embodiments, the aqueous buffer solution has a pH of about 5.

In some embodiments, the aqueous buffer solution can comprise an acetate buffer, a citrate buffer, a phosphate buffer, or a Tris buffer. In some embodiments, the aqueous buffer solution comprises an acetate buffer or a citrate buffer. In further embodiments, the aqueous buffer solution is an acetate buffer, such as a sodium acetate buffer.

In some embodiments, the aqueous buffer solution has a buffer concentration greater than about 30 mM. In some embodiments, the aqueous buffer solution has a buffer concentration greater than about 40 mM. In some embodiments, the aqueous buffer solution has a buffer concentration of about 30 mM to about 100 mM. In some embodiments, the aqueous buffer solution has a buffer concentration of about 40 mM to about 75 mM. In some embodiments, the aqueous buffer solution has a buffer concentration of about 25 mM. In further embodiments, the aqueous buffer solution has a buffer concentration of about 33 mM, about 37.5 mM, or about 45 mM.

In some embodiments, the aqueous buffer solution can have an ionic strength of about 15 mM or less, about 10 mM or less, or about 5 mM or less. In some embodiments, the aqueous buffer solution has an ionic strength of about 0.1 mM to about 15 mM, about 0.1 mM to about 10 mM, or about 0.1 mM to about 5 mM.

In some embodiments, the lipid solution has a lipid concentration of about 5 to about 100 mg/mL, about 15 to about 35 mg/mL, about 20 to about 30 mg/mL, or about 24 mg/mL.

The lipid solution can further comprise an organic solvent such as an alcohol, e.g., ethanol. The organic solvent can be present in an amount of about 1% to about 50%, about 5% to about 40%, or about 10% to about 33% by volume. In further embodiments, the solvent in is 100% ethanol or greater than 95% ethanol by volume.

In some embodiments, the lipid solution comprises about 30 mol % to about 60 mol %, about 35 mol % to about 55 mol %, or about 40 mol % to about 50 mol % of ionizable lipid with respect to total lipids. In some embodiments, the lipid solution comprises about 5 mol % to about 15 mol %, about 8 mol % to about 13 mol %, or about 10 mol % to about 12 mol % of phospholipid with respect to total lipids. In some embodiments, the lipid solution comprises about 30 mol % to about 50 mol %, about 35 mol % to about 45 mol %, or about 37 mol % to about 42 mol % of structural lipid with respect to total lipids. In some embodiments, the lipid solution comprises about 0.1 mol % to about 2 mol %, about 0.1 mol % to about 1 mol %, or about 0.25 mol % to about 0.75 mol % of PEG-lipid with respect to total lipids.

In some embodiments, the lipid solution comprises:
about 40 mol % to about 50 mol % of ionizable lipid;
about 10 mol % to about 12 mol % of phospholipid;
about 37 mol % to about 42 mol % of structural lipid; and
about 0.25 mol % to about 0.75 mol % of PEG-lipid; each with respect to total lipids.

In some embodiments, the lipid solution comprises:
about 49 mol % of ionizable lipid;
about 11 mol % to about 12 mol % of phospholipid;
about 39 mol % of structural lipid; and
about 0.5 mol % of PEG-lipid; each with respect to total lipids.

The mixing of the lipid solution and buffer solution results in precipitation of the lipid nanoparticles and preparation of the herein described empty lipid nanoparticle compositions. Precipitation can be carried out by ethanol-drop precipitation using, for example, high energy mixers (e.g., T-junction, confined impinging jets, microfluidic mixers, vortex mixers) to introduce lipids (in ethanol) to a suitable anti-solvent (i.e. water) in a controllable fashion, driving liquid supersaturation and spontaneous precipitation into lipid particles. In some embodiments, the mixing is carried out with a multi-inlet vortex mixer. In some embodiments, the mixing is carried out with a microfluidic mixer, such as described in WO 2014/172045. The mixing step can be performed at ambient temperature or, for example, at a temperature of less than about 30° C., less than about 28° C., less than about 26° C., less than about 25° C., less than about 24° C., less than about 22° C., or less than about 20° C.

In some embodiments, the mixing comprises nanoprecipitation. Nanoprecipitation is the unit operation in which the nanoparticles are self-assembled from their individual lipid components by way of kinetic mixing and subsequent maturation and continuous dilution. This unit operation includes three individual steps: mixing of the aqueous and organic inputs, maturation of the nanoparticles, and dilution after a controlled residence time. Due to the continuous nature of these steps, they are considered one unit operation. The unit operation includes the continuous inline combination of three liquid streams with one inline maturation step: mixing of the aqueous buffer with lipid stock solution, maturation via controlled residence time, and dilution of the nanoparticles. The nanoprecipitation itself occurs in the scale-appropriate mixer, which is designed to allow continuous, high-energy, combination of the aqueous solution with the lipid stock solution dissolved in ethanol. The aqueous solution and the lipid stock solution both flow simultaneously into the mixing hardware continuously throughout this operation. The ethanol content, which keeps the lipids dissolved, is abruptly reduced and the lipids all precipitate with each other. The particles are thus self-assembled in the mixing chamber. One of the objectives of unit operation is to exchange the solution into a fully aqueous buffer, free of ethanol, and to reach a target concentration of nanoparticle. This can be achieved by first reaching a target processing concentration, then using diafiltration, and then (if necessary) a final concentration step once the ethanol has been completely removed.

In some embodiments, the lipid nanoparticle core, which is contacted with the lipid amine, comprises the PEG-lipid. In some embodiments, the lipid nanoparticle core, which is contacted with the lipid amine, is substantially free of PEG-lipid. In some embodiments, the PEG-lipid is added to the lipid nanoparticle together with the lipid amine, prior to the contacting with the lipid amine, or after the contacting with the lipid amine. In some embodiments, the PEG-lipid is used as a stabilizer.

In some embodiments, the contacting of step (b) is carried out at a pH of about 3.5 to about 6.5. In some embodiments, the combining is carried out at a pH of about 5. In some embodiments, the pH of the empty lipid nanoparticle composition is adjusted to about 4.5 to about 5.5 prior to combining the empty lipid nanoparticle composition with payload. In some embodiments, the pH of the empty lipid nanoparticle composition is adjusted to about 5 prior to combining the empty lipid nanoparticle composition with payload.

In some embodiments, the aqueous input further comprises a payload. In some embodiments, the payload is a nucleic acid such as RNA or DNA. In some embodiments, the RNA is mRNA. In some embodiments, the aqueous input can include the nucleic acid at a concentration of about 0.05 to about 5.0 mg/mL, 0.05 to about 2.0 mg/mL, about 0.05 to about 1.0 mg/mL, about 0.1 to about 0.5 mg/mL, or about 0.2 to about 0.3 mg/mL. In some embodiments, the nucleic acid concentration is about 0.25 mg/mL. The nucleic acid payload can be provided as a nucleic acid solution comprising (i) a nucleic acid, such as DNA or RNA (e.g., mRNA), and (ii) a buffer capable of maintaining acidic pH, such as a pH of about 3 to about 6, about 4 to about 6, or about 5 to about 6. In some embodiments, the pH of the nucleic acid solution is about 5.

The mixing of the aqueous and organic inputs can occur in a scale-appropriate mixer, which is designed to allow continuous, high-energy, combination of the aqueous input with the organic input. In some embodiments, the aqueous input and organic input flow simultaneously into the mixing hardware continuously throughout this operation. In some embodiments, the aqueous input and organic input are mixed at a volume ratio of about 5:1, about 4:1, about 3:1, about 2:1, about 1:1, about 1:2, about 1:3, about 1:4, or about 1:4 aqueous input to organic input. The precipitation of the lipid amine and one or more additional lipids can be caused by reducing the organic solvent content.

In some embodiments, the maturation comprises controlled residence time. In some embodiments, the residence time is about 5 to about 120 seconds, about 10 to about 90 seconds, about 20 to about 70 seconds, about 30 to about 60 seconds, about 30 seconds, about 45 seconds, or about 60 seconds.

In some embodiments, the nanoparticles are diluted with a dilution buffer. The dilution buffer can be an aqueous buffer solution with a buffer concentration of about 0.1 mM to about 100 mM, about 0.5 mM to about 90 mM, about 1.0 mM to about 80 mM, about 2 mM to about 70 mM, about 3 mM to about 60 mM, about 4 mM to about 50 mM, about 5 mM to about 40 mM, about 6 mM to about 30 mM, about 7 mM to about 20 mM, about 8 mM to about 15 mM, or about 9 mM to about 12 mM. In some embodiments, the buffer concentration is about 30 mM to about 75 mM, about 30 mM to about 60 mM, or about 30 mM to about 50 mM. In some embodiments, the dilution buffer comprises an acetate buffer, a citrate buffer, a phosphate buffer, or a tris buffer. In some embodiments, the dilution buffer comprises an acetate buffer or a citrate buffer. In further embodiments, the dilution buffer is an acetate buffer, such as a sodium acetate. In some embodiments, the pH of the dilution buffer is about 3 to about 7, about 3 to about 6, about 3 to about 5, about 4, about 5, about 5.5, or about 6. In some embodiments, the dilution buffer comprises the same buffer as in the aqueous input.

In some embodiments, the process of preparing a lipid nanoparticle composition further comprises filtering. In some embodiments, the filtering comprises dialysis. In some embodiments, the dialysis is performed using a Slide-A-Lyzer dialysis cassette. In some embodiments, the dialysis cassette has a molecular weight cut off of about 5 kDa, about 10 kDa, about 15 kDa, or about 20 kDa. The dialysis can be carried out at about 25° C., about 20° C., about 10° C., about 5° C., or about 4° C. In some embodiments, the filtering further comprises filtering through a 0.1 μm to about 1 μm filter. In some embodiments, the filtering further comprises filtering through a 0.22 μm filter.

In some embodiments, the buffer of the nucleic acid solution is an acetate buffer, a citrate buffer, a phosphate buffer, or a tris buffer. In some embodiments, the buffer is an acetate buffer or a citrate buffer.

In further embodiments, the buffer is an acetate buffer, such as a sodium acetate buffer. The buffer concentration of the nucleic acid solution can be about 5 mM to about 140 mM. In some embodiments, the buffer concentration is about 20 mM to about 100 mM, about 30 mM to about 70 mM, or about 40 mM to about 50 mM. In some embodiments, the buffer concentration is about 42.5 mM.

The nucleic acid solution can include the nucleic acid at a concentration of about 0.05 to about 5.0 mg/mL, 0.05 to about 2.0 mg/mL, about 0.05 to about 1.0 mg/mL, about 0.1 to about 0.5 mg/mL, or about 0.2 to about 0.3 mg/mL. In some embodiments, the nucleic acid concentration is about 0.25 mg/mL.

High energy mixers (e.g., T-junction, confined impinging jets, microfluidic mixers, vortex mixers) can be used for the contacting of step (b). In some embodiments, the combining is carried out with a multi-inlet vortex mixer. In some embodiments, the combining is carried out with a microfluidic mixer, such as described in WO 2014/172045. The combining step can be performed at ambient temperature or, for example, at a temperature of less than about 30° C., less than about 28° C., less than about 26° C., less than about 25° C., less than about 24° C., less than about 22° C., or less than about 20° C.

In some embodiments, the contacting of the LNP core with a lipid amine comprises dissolving the lipid amine in a non-ionic excipient. In some embodiments, the non-ionic excipient is selected from macrogol 15-hydroxystearate (HS 15), 1,2-dimyristoyl-rac-glycero-3-methoxypolyethylene glycol-2000 (PEG-DMG-2K), PL1, polyoxyethylene sorbitan monooleate [TWEEN®80], and d-α-Tocopherol polyethylene glycol succinate (TPGS). In some embodiments, the non-ionic excipient is macrogol 15-hydroxystearate (HS 15).

In some embodiments, the contacting of the lipid nanoparticle core with a lipid amine comprises the lipid amine dissolved in a buffer solution. In some embodiments, the buffer is an acetate buffer, a citrate buffer, a phosphate buffer, or a tris buffer. In some embodiments, the buffer solution is a phosphate buffered saline (PBS). In some embodiments, the buffer solution is a Tris-based buffer. In some embodiments, the buffer solution concentration is about 5 mM to about 100 mM, about 5 mM to about 50 mM, about 10 mM to about 30 mM, or about 20 mM.

In some embodiments, the lipid amine solution has a pH of about 7 to about 8, or about 7.5. In some embodiments, the concentration of the lipid amine solution is about 0.1 to about 50 mg/mL, about 1 to about 30 mg/mL, about 1 to about 10 mg/mL, or about 2 to about 3 mg/mL.

In some embodiments, the lipid nanoparticle composition undergoes maturation via controlled residence time after loading and prior to neutralization. In some embodiments, the residence time is about to about 120 seconds, about 10 to about 90 seconds, about 20 to about 70 seconds, about 30 to about 60 seconds, about 30 seconds, about 45 seconds, or about 60 seconds.

In some embodiments, the lipid nanoparticle composition undergoes maturation via controlled residence time after neutralization and prior to addition of cationic agent. In some embodiments, the residence time is about 1 to about 30 seconds, about 2 to about 20 seconds, about 5 to about 15 seconds, about 7 to about 12 seconds, or about 10 seconds.

In some embodiments, the processes of preparing lipid nanoparticle compositions further comprise one or more additional steps selected from:
diluting the composition with a dilution buffer;
adjusting the pH of the composition;
adding one or more surface-acting agents to the composition;
filtering the composition;
concentrating the composition;
exchanging buffer of the composition;
adding cryoprotectant to the composition; and
adding an osmolality modifier to the composition.

In some embodiments, the processes of preparing lipid nanoparticle compositions can further comprise 1, 2, 3, 4, 5, 6, 7, or all of the above-listed steps. Some steps may be repeated. The steps can be, but need not be, carried out in the order listed. Each of the steps refers to an action relating to the composition that results from the prior enacted step. For example, if the process includes the step of adding one or more surface-acting agents to the composition, then the surface-acting agent is added to the composition resulting from the previous step, where the previous step could be any of the above-listed steps.

In some embodiments, the one or more additional steps is adjusting the pH of the composition to a pH of about 7 to about 8. In some embodiments, the pH is adjusted to a pH of about 7.5.

In some embodiments, the one or more additional steps is adding a further surface-acting agent to the filled lipid nanoparticle (e.g., in addition to the lipid amine). A surface-acting agent may be disposed within a nanoparticle and/or on its surface (e.g., by coating, adsorption, covalent linkage, or other process). Surface-acting agents may include, but are not limited to, PEG derivatives (e.g., PEG-DMG), lipid amines (e.g. sterol amines and related), anionic proteins (e.g., bovine serum albumin), surfactants (e.g., cationic surfactants such as dimethyldioctadecylammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). In some embodiments, the further surface-acting agent is a PEG lipid, such as PEG-DMG. In some embodiments, the further surface-acting agent is provided together with the lipid amine. In some embodiments, the further surface-acting agent is present together with the lipid amine in the lipid amine solution. In some embodiments, the further surface-acting agent is a PEG-lipid having a concentration of about 0.1 to about 50 mg/mL, about 1 to about 10 mg/mL, or about 1 to about 3 mg/mL.

In some embodiments, the one or more additional step is adding an osmolality modifier to the composition. The osmolality modifier can be a salt or a sugar. In some embodiments, the osmolality modifier is a sugar. The sugar can be selected from, but not limited to glucose, fructose, galactose, sucrose, lactose, maltose, and dextrose. In some embodiments, the osmolality modifier is a salt. The salt can be an inorganic salt, e.g., sodium chloride, potassium chloride, calcium chloride, or magnesium chloride. In some embodiments, the inorganic salt is sodium chloride. In some embodiments, the salt is 4-(2-hydroxyethyl)piperazine-1-ethanesulfonic acid sodium salt. The salt can be provided as a salt solution having a salt concentration of about 100 to about 500 mM, about 200 to about 400 mM, about 250 to about 350 mM, or about 300 mM. The pH of the salt solution can be about 7 to about 8. The salt solution can further include a buffer comprising, for example, an acetate buffer, a citrate buffer, a phosphate buffer, or a tris buffer. The buffer concentration can be, for example, about 0.1 mM to about 100 mM, about 0.5 mM to about 90 mM, about 1.0 mM to about 80 mM, about 2 mM to about 70 mM, about 3 mM to about 60 mM, about 4 mM to about 50 mM, about 5 mM to about 40 mM, about 6 mM to about 30 mM, about 7 mM to about 20 mM, about 8 mM to about 15 mM, or about 9 mM to about 12 mM.

Cryoprotectant can be added to the filled nanoparticle composition by the addition of an aqueous cryoprotectant solution which can include an aqueous buffer with a buffer concentration of about 0.1 mM to about 100 mM, about 0.5 mM to about 90 mM, about 1.0 mM to about 80 mM, about 2 mM to about 70 mM, about 3 mM to about 60 mM, about 4 mM to about 50 mM, about 5 mM to about 40 mM, about 6 mM to about 30 mM, about 7 mM to about 20 mM, about 8 mM to about 15 mM, or about 9 mM to about 12 mM. In some embodiments, the buffer concentration is about 1 to 20 mM about 1 to about 10 mM, or about 5 mM. In some embodiments, the buffer in the cryoprotectant solution comprises an acetate buffer, a citrate buffer, a phosphate buffer, or a tris buffer. In some embodiments, the buffer is an acetate buffer or a citrate buffer. In further embodiments, the buffer is an acetate buffer, such as a sodium acetate. In some embodiments, the pH of the cryoprotectant solution is about 7 to about 8, such as about 7.5. In some embodiments, the cryoprotectant solution comprises about 40% to about 90%, about 50% to about 85%, about 60% to about 80%, or about 70% by weight of sucrose.

In some embodiments, the processes of the invention further include the step of diluting the composition with a dilution buffer. The dilution buffer can be an aqueous buffer solution with a buffer concentration of about 0.1 mM to about 100 mM, about 0.5 mM to about 90 mM, about 1.0 mM to about 80 mM, about 2 mM to about 70 mM, about 3 mM to about 60 mM, about 4 mM to about 50 mM, about 5 mM to about 40 mM, about 6 mM to about 30 mM, about 7 mM to about 20 mM, about 8 mM to about 15 mM, or about 9 mM to about 12 mM. In some embodiments, the buffer concentration is about 30 mM to about 75 mM, about 30 mM to about 60 mM, or about 30 mM to about 50 mM. In some embodiments, the dilution buffer comprises an acetate buffer, a citrate buffer, a phosphate buffer, or a tris buffer. In some embodiments, the dilution buffer comprises an acetate buffer or a citrate buffer. In further embodiments, the dilution buffer is an acetate buffer, such as a sodium acetate. In some embodiments, the pH of the dilution buffer is about 3 to about 7, about 3 to about 6, about 3 to about 5, about 4, about 5, about 5.5, or about 6. In some embodiments, the dilution buffer comprises the same buffer as in the aqueous buffer solution used during the combining of the of the empty lipid nanoparticle composition with the nucleic acid solution.

In some embodiments, the processes of the invention further include any one or more of the steps of: filtering the composition; concentrating the composition; and exchanging buffer of the composition. The filtration, concentration, and buffer exchange steps can be accomplished with tangential flow filtration (TFF). Residual organic solvent can be removed by the filtration step.

In some embodiments, buffer exchange can change the composition of the filled lipid nanoparticle composition by raising or lowering buffer concentration, changing buffer composition, or changing pH.

In some embodiments, the concentration step can increase the concentration of the filled lipid nanoparticles in the composition.

In some embodiments, the processes of preparing filled lipid nanoparticle compositions further comprise at least the steps of: adjusting the pH of the composition to a pH of about 7 to about 8 (e.g., about pH 7.5); and adding an osmolality modifier (e.g., an inorganic salt) to the composition.

In some embodiments, the processes of preparing filled lipid nanoparticle compositions further comprise at least the steps of: adjusting the pH of the composition to a pH of about 7 to about 8 (e.g., about pH 7.5); adding a surface-acting agent to the composition; and adding an osmolality modifier (e.g., an inorganic salt) to the composition.

In some embodiments, the processes of preparing lipid nanoparticle compositions can further include:
(i) adjusting the pH of the composition to a pH of about 7 to about 8;
(ii) adding one or more surface-acting agents to the composition;
(iii) concentrating the composition;
(iv) adding an inorganic salt to the composition; and
(v) diluting the composition.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the schemes below.

The reactions for preparing compounds described herein can be carried out in suitable solvents, which can be readily selected by one of skill in the art of organic synthesis. Suitable solvents can be substantially non-reactive with the starting materials (reactants), the intermediates, or products at the temperatures at which the reactions are carried out, (e.g., temperatures, which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

Compounds of Formula A2a can be prepared, e.g., using a process as illustrated in the schemes below:

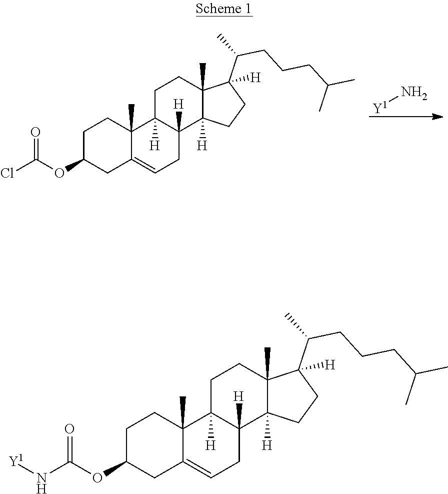

Compounds of Formula A2a can be prepared via the synthetic route outlined in Scheme 1. An appropriate reaction between cholesteryl chloroformate and amines can be carried out under suitable conditions to generate a compound of Formula A2a.

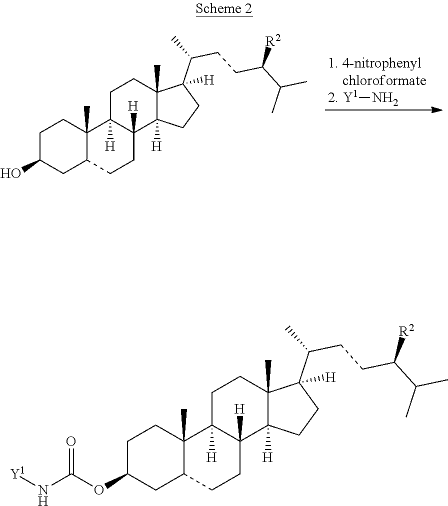

Compounds of Formula A2a can be prepared via the synthetic route outlined in Scheme 2. An appropriate reaction between cholesterol or a cholesterol derivative (such as stigmasterol) and 4-nitrophenyl chloroformate can be carried out under suitable conditions (such as using triethylamine and 4-dimethylaminopyridine). The product of said reaction can be reacted with an amine under suitable conditions (such as using triethylamine) to give a compound of Formula A2a.

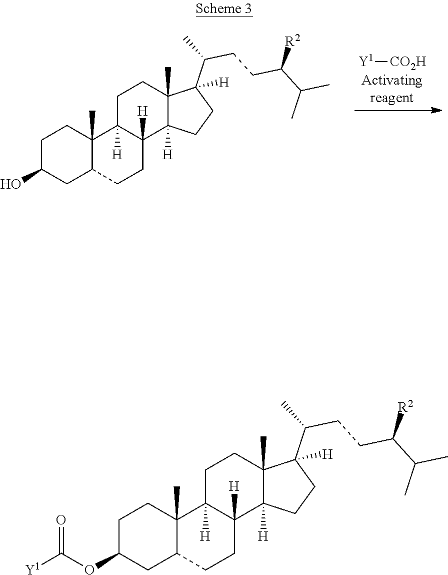

Compounds of Formula A2a can be prepared via the synthetic route outlined in Scheme 3. An appropriate reaction between cholesterol or a cholesterol derivative (such as stigmasterol) and a carboxylic acid can be carried out in the presences of an activating reagent (such as, e.g., EDC-HCl, DMAP, DCC, or pivalic anhydride) in suitable conditions to give compounds of Formula A2a.

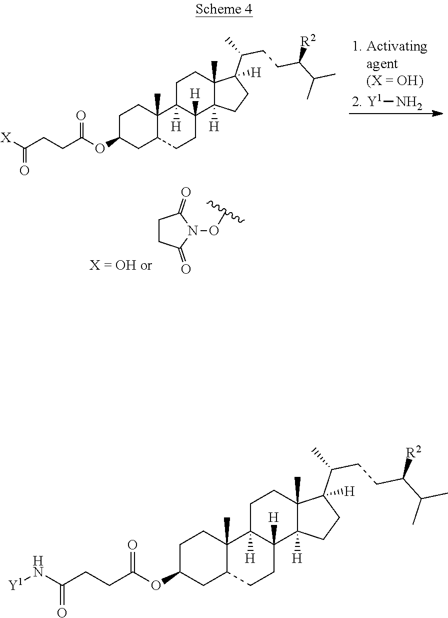

Compounds of Formula A2a can be prepared via the synthetic route outlined in Scheme 4. An appropriate reaction between cholesterol hemisuccinate or a cholesterol derivative hemisuccinate and an activating agent can be carried out under suitable conditions. The product of said reaction can be reacted with an amine under suitable conditions to give compounds of Formula A2a.

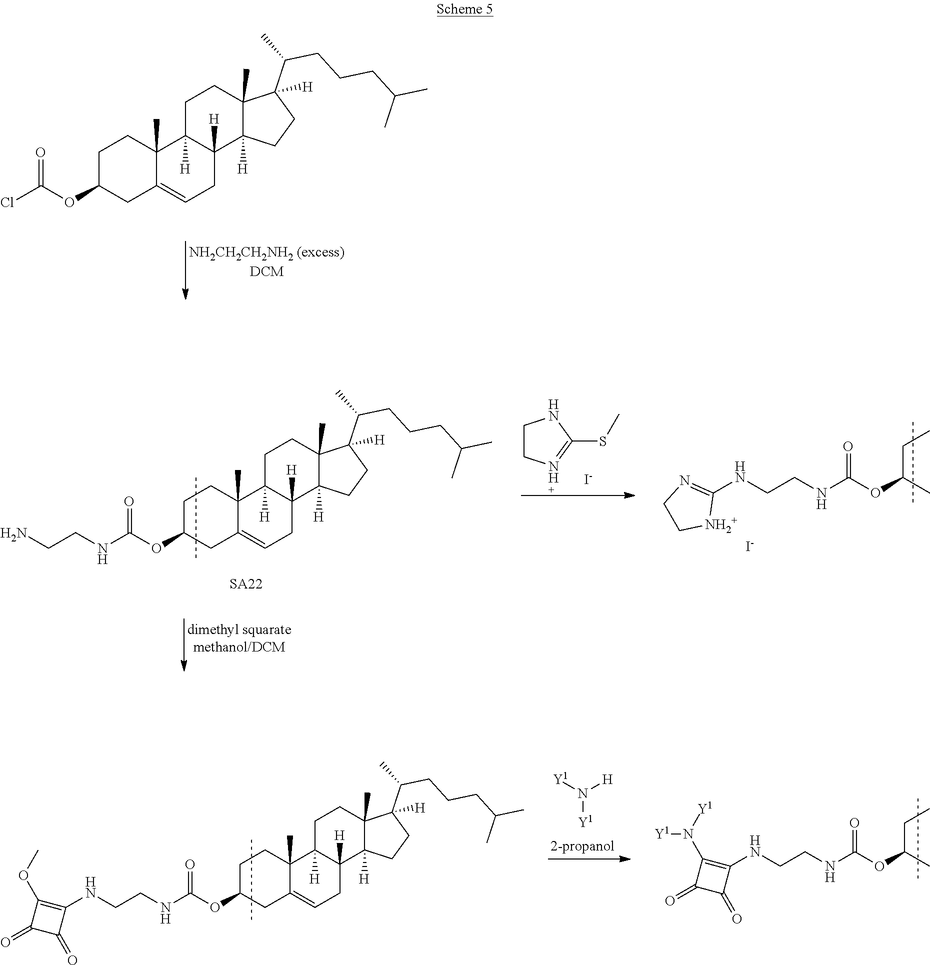

Compounds of Formula A2a can be prepared via the synthetic route outlined in Scheme 5. An appropriate reaction between cholesteryl chloroformate and ethane-1,2-diamine can be carried out under suitable conditions to give a SA22. SA22 can be reacted with 2-(methylthio)-4,5-dihydro-1H-imidazole hydroiodide under suitable conditions to give a compound of Formula A2a. SA22 can also be reacted with dimethyl squarate under suitable conditions, and the product of the reaction can be further reacted with a secondary amine under suitable conditions to give a compound of Formula A2a.

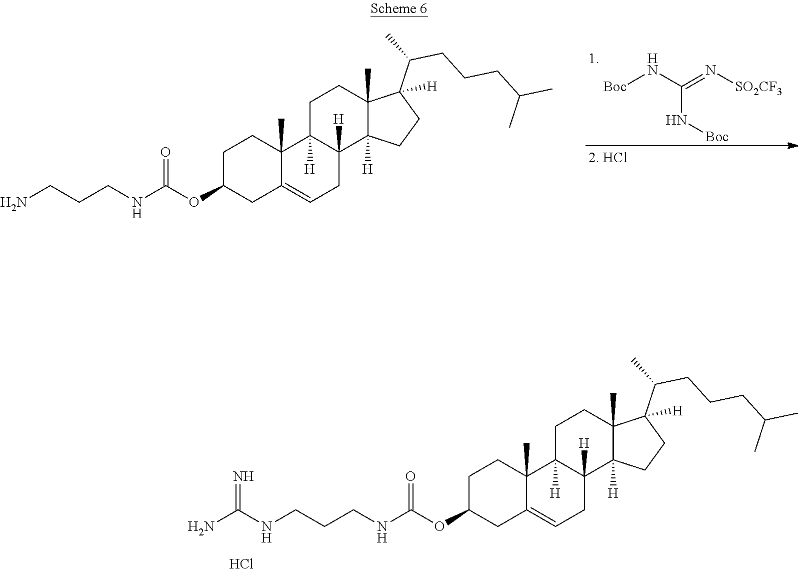

Compounds of Formula A2a can be prepared via the synthetic route outlined in Scheme 6. An appropriate reaction between an aminoalkyl carbamate and a guanidinylation agent can be carried out under suitable conditions. The product of said reaction can be reacted with HCl under suitable conditions to give a compound of Formula A2a.

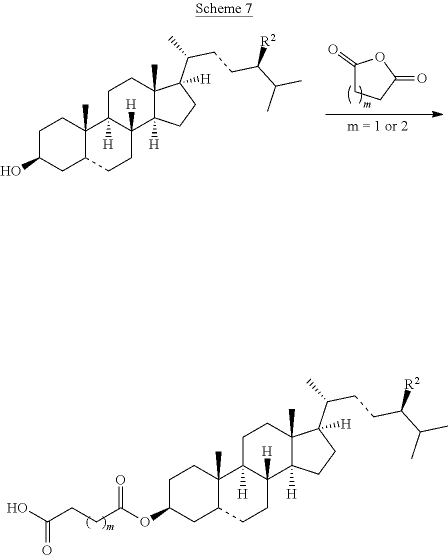

Precursors to compounds of Formula A2a can be prepared via the synthetic route outlined in Scheme 7. An appropriate reaction between cholesterol or a cholesterol derivative (such as stigmasterol) and can be carried out under suitable conditions (such as using triethylamine and 4-dimethylaminopyridine). The product of said reaction can be reacted with an amine under suitable conditions (such as using triethylamine) to give a precursor to a compound of Formula A2a.

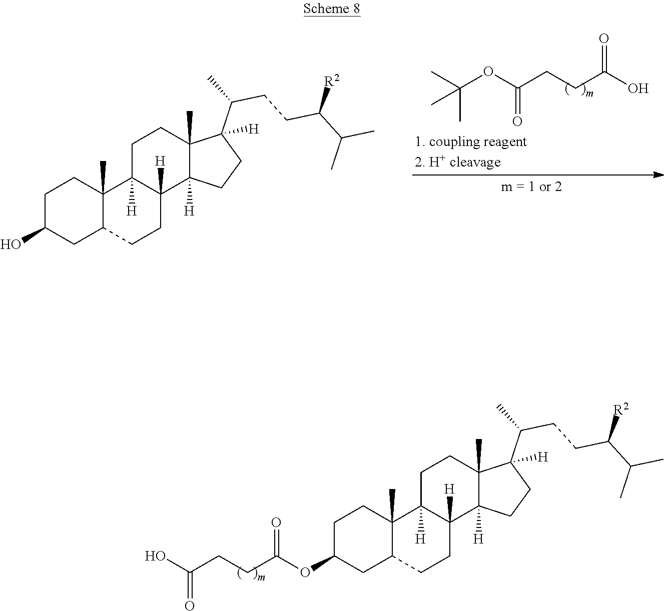

Precursors to compounds of Formula A2a can be prepared via the synthetic route outlined in Scheme 8. An appropriate reaction between cholesterol or a cholesterol derivative (such as stigmasterol) and a boc-hemiester can be carried out under suitable conditions. The product of said reaction can be reacted under suitable conditions to give a precursor to a compound of Formula A2a.

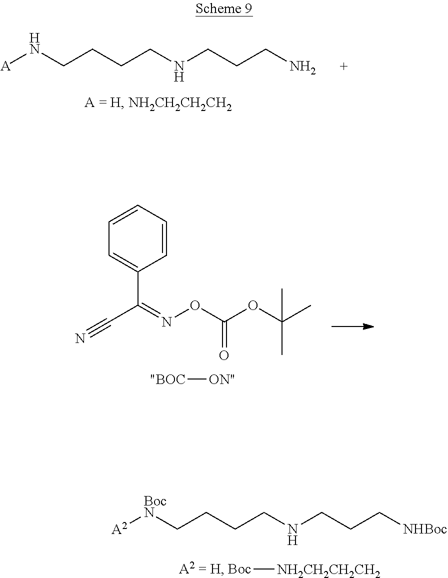

Intermediates for the synthesis of compounds of Formula A2a can be prepared via the synthetic route outlined in Scheme 9. An appropriate reaction between spermidine or spermine and (E)-N—((tert-butoxycarbonyl)oxy)benzimidoyl cyanide (BOC—ON) can be carried out under suitable conditions to give an intermediate for the synthesis of compounds of Formula A2a.

Compounds of Formula A6 can be prepared, e.g., using a process as illustrated in the schemes below:

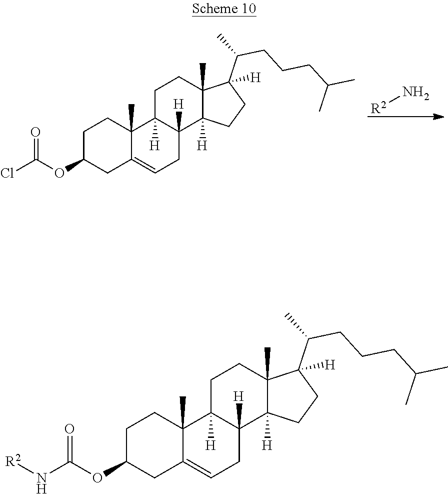

Compounds of Formula A6 can be prepared via the synthetic route outlined in Scheme 10. An appropriate reaction between cholesteryl chloroformate and amines can be carried out under suitable conditions to generate a precursor to a compound of Formula A6 or a compound of Formula A6.

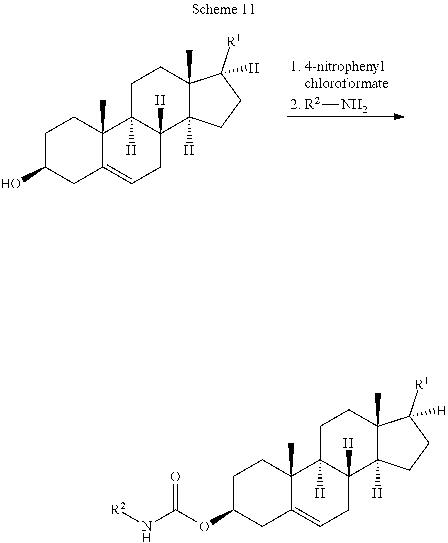

Compounds of Formula A6 can be prepared via the synthetic route outlined in Scheme 11. An appropriate reaction between cholesterol or a cholesterol derivative (such as stigmasterol) and 4-nitrophenyl chloroformate can be carried out under suitable conditions (such as using triethylamine and 4-dimethylaminopyridine). The product of said reaction can be reacted with an amine under suitable conditions (such as using triethylamine) to generate a precursor to a compound of Formula A6 or a compound of Formula A6.

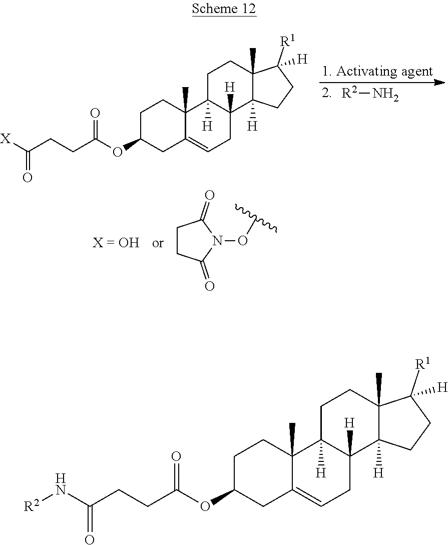

Compounds of Formula A6 can be prepared via the synthetic route outlined in Scheme 12. An appropriate reaction between cholesterol hemisuccinate or a cholesterol derivative hemisuccinate and an activating agent can be carried out under suitable conditions. The product of said reaction can be reacted with an amine under suitable conditions to generate a precursor to a compound of Formula A6 or a compound of Formula A6.

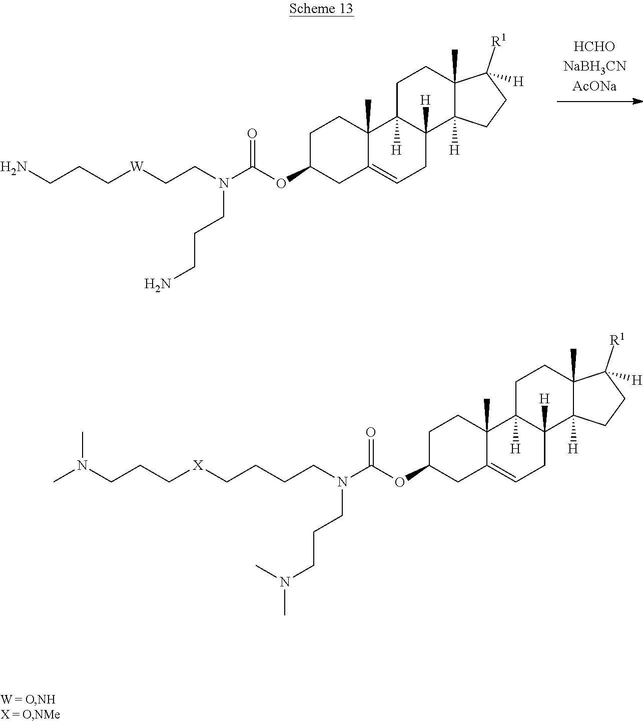

Compounds of Formula A6 can be prepared via the synthetic route outlined in Scheme 13. An appropriate reaction between a compound of Formula A6, HCHO, NaBH₃CN, and AcONa can be carried out under suitable conditions to generate a compound of Formula A6.

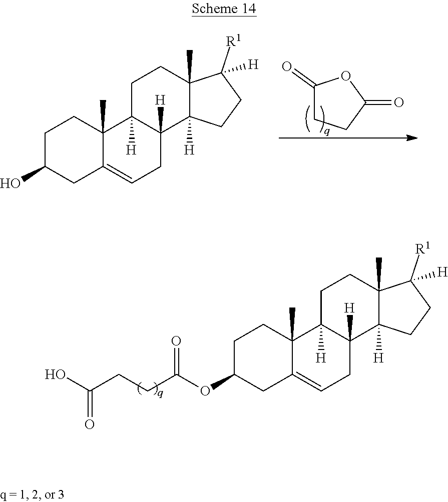

Precursors to compounds of Formula A6 can be prepared via the synthetic route outlined in Scheme 14. An appropriate reaction between cholesterol or a cholesterol derivative (such as stigmasterol) and can be carried out under suitable conditions (such as using triethylamine and 4-dimethylaminopyridine). The product of said reaction can be reacted with an amine under suitable conditions (such as using triethylamine) to give a precursor to a compound of Formula A6.

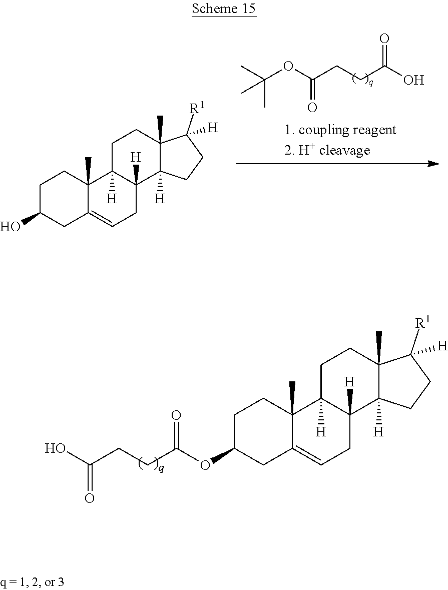

Precursors to compounds of Formula A6 can be prepared via the synthetic route outlined in Scheme 15. An appropriate reaction between cholesterol or a cholesterol derivative (such as stigmasterol) and a boc-hemiester can be carried out under suitable conditions. The product of said reaction can be reacted under suitable conditions to give a precursor to a compound of Formula A6.

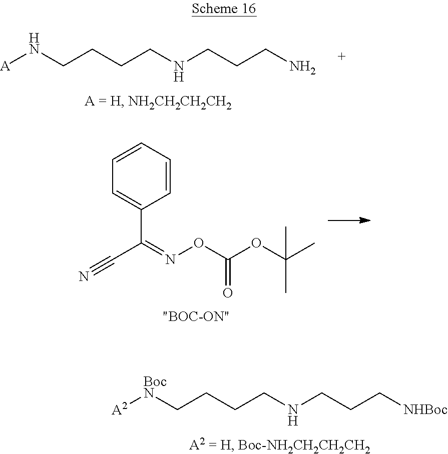

Intermediates for the synthesis of compounds of Formula A6 can be prepared via the synthetic route outlined in Scheme 16. An appropriate reaction between spermidine or spermine and (E)-N—((tert-butoxycarbonyl)oxy)benzimidoyl cyanide (BOC—ON) can be carried out under suitable conditions to give an intermediate for the synthesis of compounds of Formula A6.

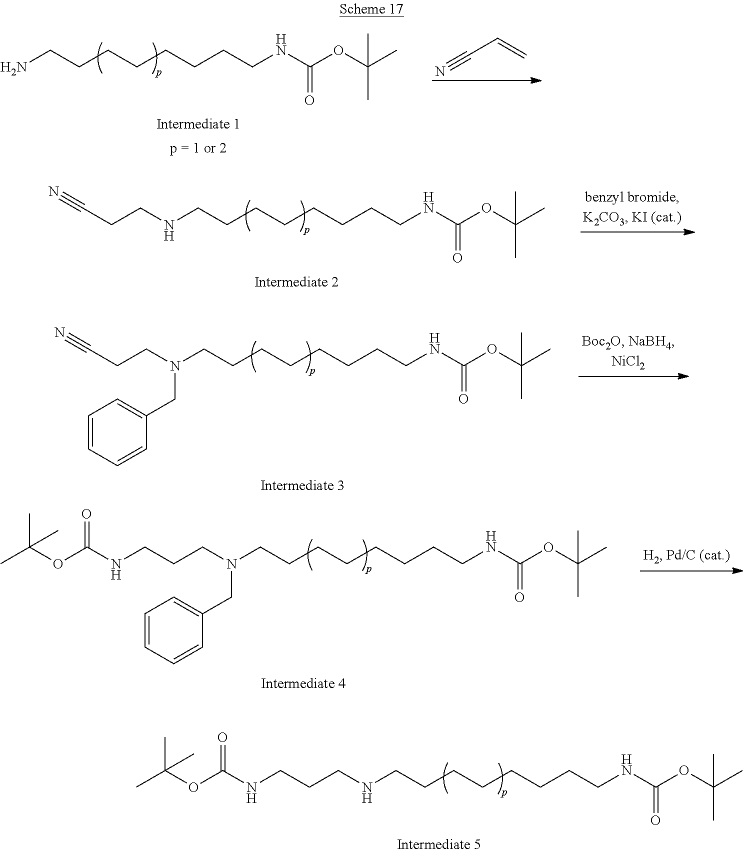

Intermediates for the synthesis of compounds of Formula A6 can be prepared via the synthetic route outlined in Scheme 17. An appropriate reaction between Intermediate 1 and acrylonitrile can be carried out under suitable conditions to give Intermediate 2. Intermediate 2 can be reacted with benzyl bromide under suitable conditions (such as, e.g. K₂CO₃ and KI) to give Intermediate 3. Intermediate 3 can be reacted with Boc₂O under suitable conditions (such as, e.g. NaBH₄ and NiCl₂) to give Intermediate 4. The benzyl group of Intermediate 4 can be removed under suitable conditions (such as H₂ and Pd/C) to give Intermediate 5.

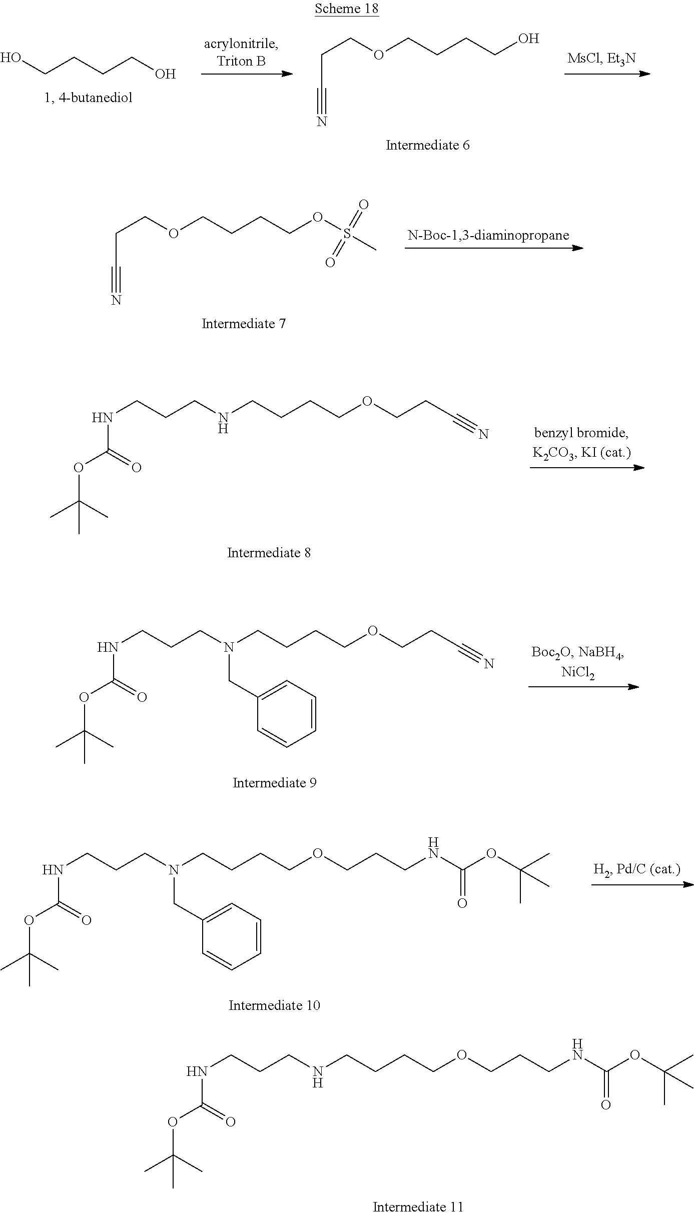

Intermediates for the synthesis of compounds of Formula A6 can be prepared via the synthetic route outlined in Scheme 18. An appropriate reaction between 1,4-butanediol and acrylonitrile can be carried out under suitable conditions (such as, e.g. Triton B) to give Intermediate 6. Intermediate 6 can be reacted with methanesulfonyl chloride under suitable conditions (such as, e.g. triethylamine) to give Intermediate 7. Intermediate 7 can be reacted with N-Boc-1,3-diaminopropane under suitable conditions to give intermediate 8. Intermediate 8 can be reacted with benzyl bromide under suitable conditions (such as, e.g. K₂CO₃ and KI) to give Intermediate 9. Intermediate 9 can be reacted with Boc₂O under suitable conditions (such as, e.g. NaBH₄ and NiCl2) to give Intermediate 10. The benzyl group of Intermediate 10 can be removed under suitable conditions (such as, e.g. H₂ and Pd/C) to give Intermediate 11.

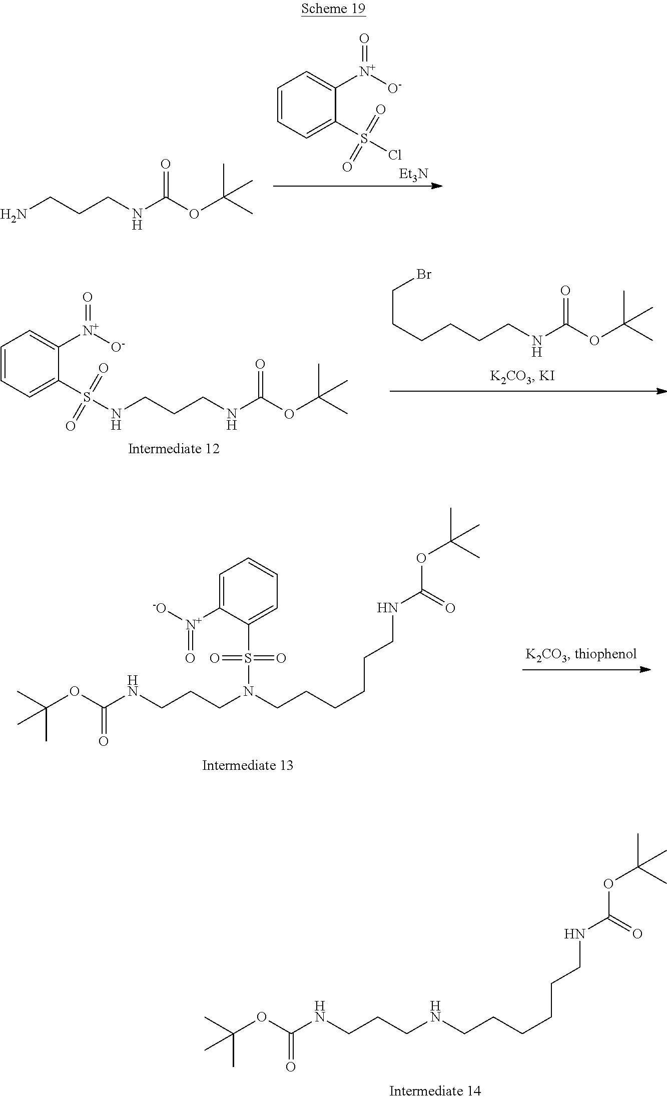

Intermediates for the synthesis of compounds of Formula A6 can be prepared via the synthetic route outlined in Scheme 19. An appropriate reaction between N-Boc-1,3-diaminopropane and 2-nitrobenzenesulfonyl chloride under suitable conditions (such as, e.g. triethylamine) to give Intermediate 12. Intermediate 12 can be reacted with tert-butyl N-(6-bromohexyl)carbamate under suitable conditions (such as, e.g. K₂CO₃ and KI) to give Intermediate 13. The 2-nitrobenzenesulfonyl group can be removed under suitable conditions (such as, e.g. K₂CO₃ and thiophenol) to give Intermediate 14.

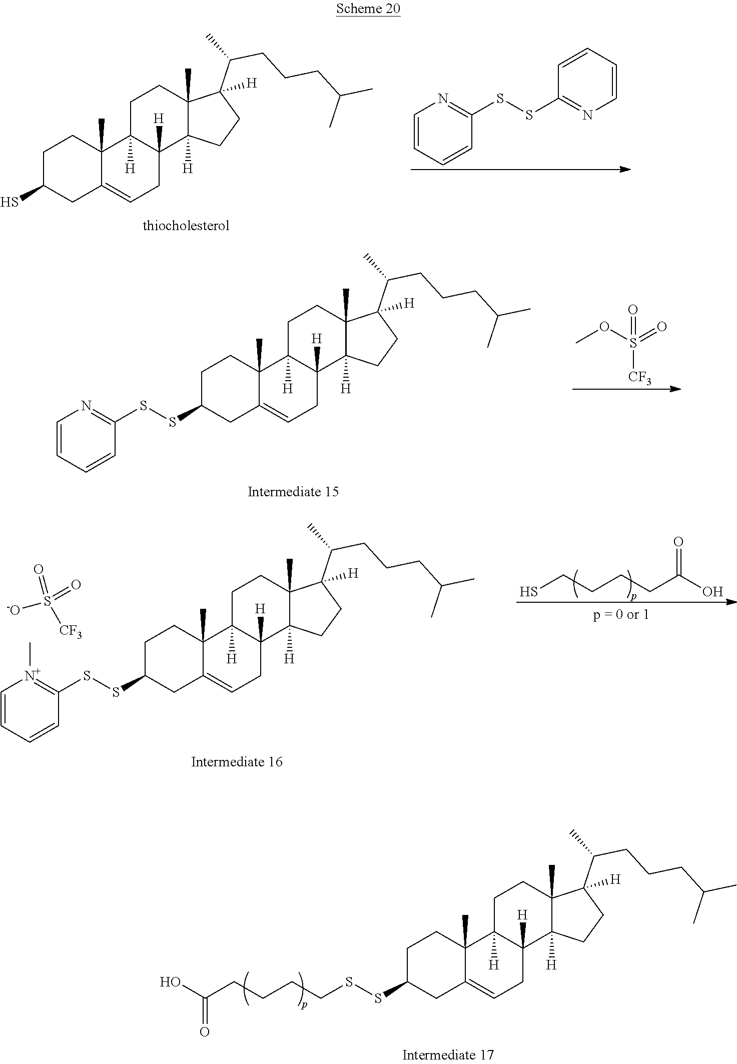

Intermediates for the synthesis of compounds of Formula A6 can be prepared via the synthetic route outlined in Scheme 20. An appropriate reaction between thiocholesterol and 2,2′-dipyridyldisulfide under suitable conditions give Intermediate 15. Intermediate 15 can be reacted with methyl trifluoromethanesulfonate (methyl triflate) under suitable conditions to give Intermediate 16. Intermediate 16 can be reacted with an appropriate mercaptocarboxylic acid to afford Intermediate 17.

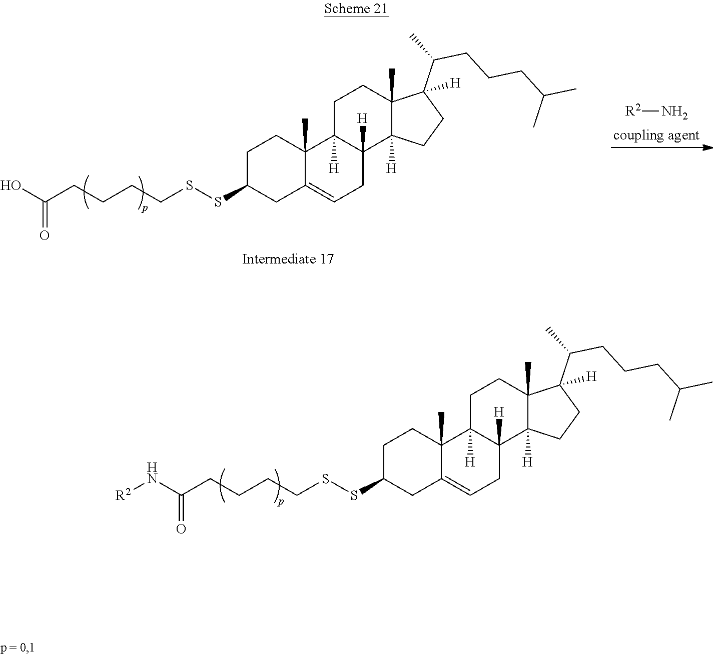

Compounds of Formula A6 can be prepared via the synthetic route outlined in Scheme 21. An appropriate reaction between Intermediate 17 and an amine can be carried out under suitable conditions (such as using a coupling agent) to generate a precursor to a compound of Formula A6 or a compound of Formula A6.

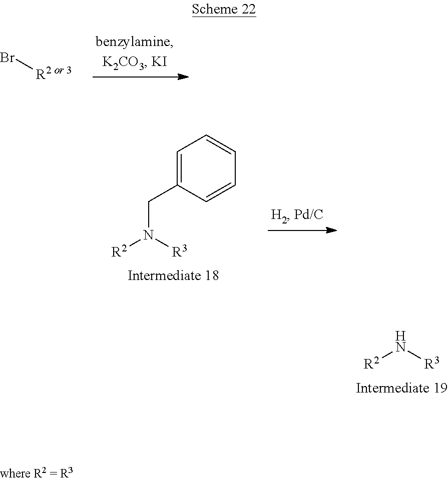

Intermediates for the synthesis of compounds of Formula A6 can be prepared via the synthetic route outlined in Scheme 22. An appropriate reaction between benzylamine and an alkyl halide under suitable conditions (such as, e.g. K₂CO₃ and KI) gives Intermediate 18. The benzyl group of Intermediate 18 is removed under suitable conditions (such as, e.g. H2 and Pd/C) to give Intermediate 19.

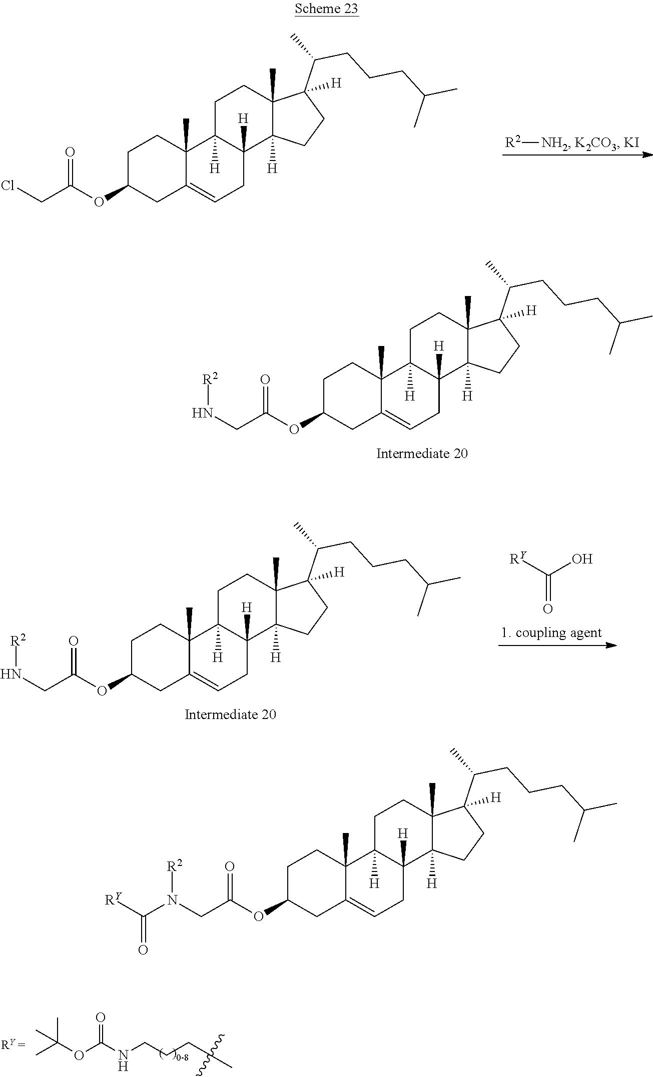

Compounds of Formula A6 or precursors for the synthesis of compounds of Formula A6 can be prepared via the synthetic route outlined in Scheme 23. An appropriate reaction between cholesterol chloroacetate and an amine under suitable conditions (such using, e.g. K₂CO₃ and KI) to give Intermediate 20. Intermediate 20 can be reacted with an appropriate carboxylic acid under suitable conditions to generate a precursor compound of Formula A6 or a compound of Formula A6. In some embodiments, R^{Y} is

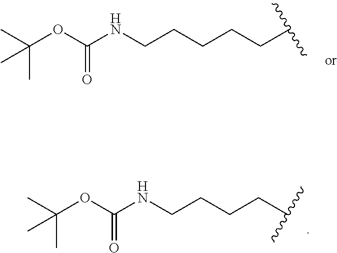 [SMILES: *CCCCCNC(=O)OC(C)(C)C ; *CCCCNC(=O)OC(C)(C)C]

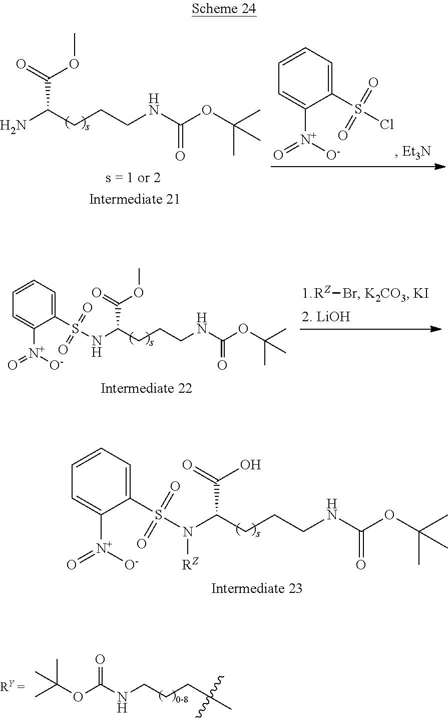

Precursors to compounds of Formula A6 can be prepared via the synthetic route outlined in Scheme 24. An appropriate between Intermediate 21 and nosyl chloride can be carried out under suitable conditions (such as, e.g., triethylamine) to give Intermediate 22. Intermediate 22 can be reacted with an alkyl bromide under suitable conditions (such as, e.g., K₂CO₃ and KI) to give Intermediate 23. In some embodiments, R^{Z} is

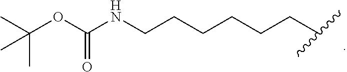 [SMILES: *CCCCCCNC(=O)OC(C)(C)C]

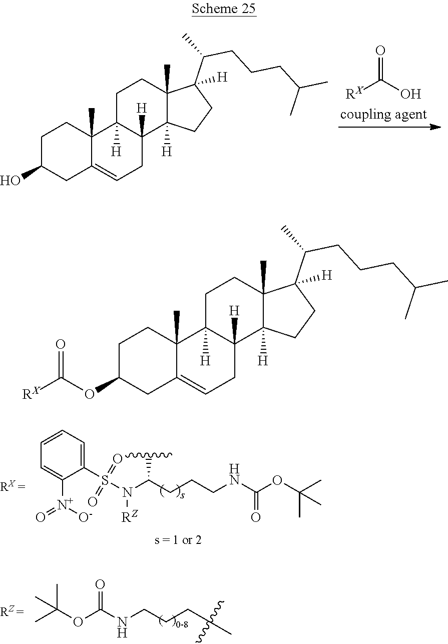

Precursors to compounds of Formula A6 can be prepared via the synthetic route outlined in Scheme 25. An appropriate reaction between cholesterol and a carboxylic acid can be carried out under suitable conditions in the presence of a coupling agent. The product of said reaction can be reacted under suitable conditions to give a compound of Formula A6 or a precursor of a compound of Formula A6. In some embodiments, R^{X} is

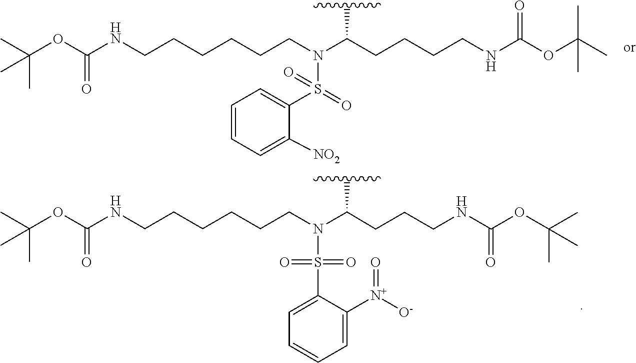

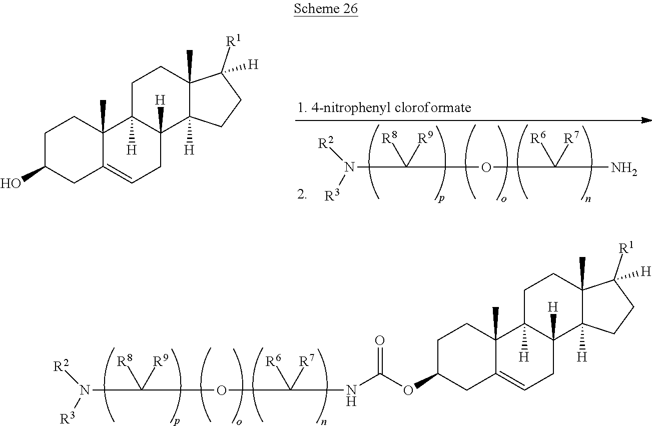

Compounds of Formula A8 can be prepared via the synthetic route outlined in Scheme 26. An appropriate reaction between cholesterol or a cholesterol derivative (such as stigmasterol) and 4-nitrophenyl chloroformate can be carried out under suitable conditions (such as using triethylamine and 4-dimethylaminopyridine). The product of said reaction can be reacted with an amine under suitable conditions (such as using triethylamine) to generate a precursor to a compound of Formula A8 or a compound of Formula A8.

### Definitions

In order that the present disclosure can be more readily understood, certain terms are first defined. As used in this application, except as otherwise expressly provided herein, each of the following terms shall have the meaning set forth below. Additional definitions are set forth throughout the application.

The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the present disclosure. Where a value is explicitly recited, it is to be understood that values which are about the same quantity or amount as the recited value are also within the scope of the present disclosure. Where a combination is disclosed, each subcombination of the elements of that combination is also specifically disclosed and is within the scope of the present disclosure. Conversely, where different elements or groups of elements are individually disclosed, combinations thereof are also disclosed. Where any element of a present disclosure is disclosed as having a plurality of alternatives, examples of that present disclosure in which each alternative is excluded singly or in any combination with the other alternatives are also hereby disclosed; more than one element of an present disclosure can have such exclusions, and all combinations of elements having such exclusions are hereby disclosed.

About: The term “about” as used in connection with a numerical value throughout the specification and the claims denotes an interval of accuracy, familiar and acceptable to a person skilled in the art. Such interval of accuracy is ±10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

Administered in combination: As used herein, the term “administered in combination” or “combined administration” means that two or more agents are administered to a subject at the same time or within an interval such that there can be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Compound: As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Contacting: As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal can be performed by varied routes of administration (e.g., intravenous, intramuscular, intradermal, and subcutaneous) and can involve varied amounts of nanoparticle compositions. Moreover, more than one mammalian cell can be contacted by a nanoparticle composition. A further example of contacting is between a nanoparticle and a cationic agent. Contacting a nanoparticle and a cationic agent can mean that the surface of the nanoparticle is put in physical connection with the cationic agent so that, the cationic agent can form a non-bonded interaction with the nanoparticle. In some embodiments, contacting a nanoparticle and a cationic agent intercalates the cationic agent into the nanoparticle, for example, starting at the surface of the nanoparticle. In some embodiments, the terms “layering,” “coating,” and “post addition” and “addition” can be used to mean “contacting” in reference to contacting a nanoparticle with a cationic agent

Delivering: As used herein, the term “delivering” means providing an entity to a destination. For example, delivering a polynucleotide to a subject can involve administering a nanoparticle composition including the polynucleotide to the subject (e.g., by an intravenous, intramuscular, intradermal, or subcutaneous route). Administration of a nanoparticle composition to a mammal or mammalian cell can involve contacting one or more cells with the nanoparticle composition.

Delivery Agent: As used herein, “delivery agent” refers to any substance that facilitates, at least in part, the in vivo, in vitro, or ex vivo delivery of a polynucleotide to targeted cells.

Diastereomer: As used herein, the term “diastereomer,” means stereoisomers that are not mirror images of one another and are non-superimposable on one another.

Disposed: As used herein, the term “disposed” means that a molecule formed a non-bonding interaction with a nanoparticle after the two were contacted with each other.

Dosing regimen: As used herein, a “dosing regimen” or a “dosing regimen” is a schedule of administration or physician determined regimen of treatment, prophylaxis, or palliative care.

Effective Amount: As used herein, the term “effective amount” of an agent is that amount sufficient to effect beneficial or desired results, for example, clinical results, and, as such, an “effective amount” depends upon the context in which it is being applied. Typically, an effective amount of a composition provides an induced or boosted immune response as a function of antigen production in the cells of the subject. In some embodiments, an effective amount of the composition containing RNA polynucleotides having at least one chemical modification are more efficient than a composition containing a corresponding unmodified polynucleotide encoding the same antigen or a peptide antigen. Increased antigen production may be demonstrated by increased cell transfection (the percentage of cells transfected with the RNA), increased protein translation and/or expression from the polynucleotide, decreased nucleic acid degradation (as demonstrated, for example, by increased duration of protein translation from a modified polynucleotide), or altered antigen specific immune response of the host cell. The term “effective amount” can be used interchangeably with “effective dose,” “therapeutically effective amount,” or “therapeutically effective dose.”

Enantiomer: As used herein, the term “enantiomer” means each individual optically active form of a compound of the present disclosure, having an optical purity or enantiomeric excess (as determined by methods standard in the art) of at least 80% (i.e., at least 90% of one enantiomer and at most 10% of the other enantiomer), at least 90%, or at least 98%.

Encapsulate: As used herein, the term “encapsulate” means to enclose, surround or encase.

Encapsulation Efficiency: As used herein, “encapsulation efficiency” refers to the amount of a polynucleotide that becomes part of a nanoparticle composition, relative to the initial total amount of polynucleotide used in the preparation of a nanoparticle composition. For example, if 97 mg of polynucleotide are encapsulated in a nanoparticle composition out of a total 100 mg of polynucleotide initially provided to the composition, the encapsulation efficiency can be given as 97%. As used herein, “encapsulation” can refer to complete, substantial, or partial enclosure, confinement, surrounding, or encasement.

Expression: As used herein, “expression” of a nucleic acid sequence refers to one or more of the following events: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

Ex Vivo: As used herein, the term “ex vivo” refers to events that occur outside of an organism (e.g., animal, plant, or microbe or cell or tissue thereof). Ex vivo events can take place in an environment minimally altered from a natural (e.g., in vivo) environment.

Helper Lipid: As used herein, the term “helper lipid” refers to a compound or molecule that includes a lipidic moiety (for insertion into a lipid layer, e.g., lipid bilayer) and a polar moiety (for interaction with physiologic solution at the surface of the lipid layer). Typically the helper lipid is a phospholipid. A function of the helper lipid is to “complement” the amino lipid and increase the fusogenicity of the bilayer and/or to help facilitate endosomal escape, e.g., of nucleic acid delivered to cells. Helper lipids are also believed to be a key structural component to the surface of the LNP.

In Vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In Vivo: As used herein, the term “in vivo” refers to events that occur within an organism (e.g., animal, plant, or microbe or cell or tissue thereof).

Ionizable amino lipid: The term “ionizable amino lipid” includes those lipids having one, two, three, or more fatty acid or fatty alkyl chains and a pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. Such ionizable amino lipids include, but are not limited to DLin-MC3-DMA (MC3) and (13Z,165Z)—N,N-dimethyl-3-nonydocosa-13-16-dien-1-amine (L608).

Isomer: As used herein, the term “isomer” means any tautomer, stereoisomer, enantiomer, or diastereomer of any compound of the present disclosure. It is recognized that the compounds of the present disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the present disclosure, the chemical structures depicted herein, and therefore the compounds of the present disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the present disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

Lipid nanoparticle core: As used herein, a lipid nanoparticle core is a lipid nanoparticle to which post addition layers of additional components can be added, such as a cationic agent and/or a PEG-lipid or other lipid. In some embodiments, the lipid nanoparticle core comprises: (i) an ionizable lipid, (ii) a phospholipid, (iii) a structural lipid, and (iv) optionally a PEG-lipid. In further embodiments, the lipid nanoparticle core comprises: (i) an ionizable lipid, (ii) a phospholipid, (iii) a structural lipid, and (iv) a PEG-lipid.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Methods of Administration: As used herein, “methods of administration” can include intravenous, intramuscular, intradermal, subcutaneous, or other methods of delivering a composition to a subject. A method of administration can be selected to target delivery (e.g., to specifically deliver) to a specific region or system of a body.

Mucosal Cells: As used herein, “mucosal cells” refer to cells which make up any mucous membrane (the moist membrane lining many tubular structures). Many are cells which provide a protective layer between the external environment and the internal organs of a subject. Examples of mucosal cells include the epithelial cells of the skin, the mucosal cells of the alimentary canal, and the tissue covering the eye. Further examples of mucosal tissue include: bronchial mucosa, endometrium, gastric mucosa, esophageal mucosa, intestinal mucosa, nasal mucosa, olfactory mucosa, oral mucosa, penile mucosa, vaginal mucosa, frenulum (of tongue), tongue, anal canal, and palpebral conjunctiva. Specific examples of mucosal cells include endocrine cells, such as K cells, L cells, S cells, G cells, D cells, I cells, Mo cells, Gr cells, and enteroendocrine cells. Non-endocrine mucosal cells include epithelial cells, mucous cells, villous cells, columnar cells, stromal cells, and paneto cells that line the outer surface of most mucosal tissues.

The term “nucleic acid,” in its broadest sense, includes any compound and/or substance that comprises a polymer of nucleotides. These polymers are often referred to as polynucleotides. Exemplary nucleic acids or polynucleotides of the present disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

Patient: As used herein, “patient” refers to a subject who can seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “solvate,” as used herein, means a compound of the present disclosure wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding mRNA sequences (comprising U) are considered codon-optimized nucleotide sequence of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a PTC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

Preventing: As used herein, the term “preventing” refers to partially or completely delaying onset of an infection, disease, disorder and/or condition; partially or completely delaying onset of one or more signs and symptoms, features, or clinical manifestations of a particular infection, disease, disorder, and/or condition; partially or completely delaying onset of one or more signs and symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune prophylaxis” refers to a measure to produce

Salts: In some aspects, the pharmaceutical composition disclosed herein and comprises salts of some of their lipid constituents. The term “salt” includes any anionic and cationic complex. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

Sample: As used herein, the term “sample” or “biological sample” refers to a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). A sample further can include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which can contain cellular components, such as proteins or nucleic acid molecule.

Single unit dose: As used herein, a “single unit dose” is a dose of any therapeutic administered in one dose/at one time/single route/single point of contact, i.e., single administration event.

Split dose: As used herein, a “split dose” is the division of single unit dose or total daily dose into two or more doses.

Stereoisomer: As used herein, the term “stereoisomer” refers to all possible different isomeric as well as conformational forms that a compound can possess (e.g., a compound of any formula described herein). This includes all possible stereochemically and conformationally isomeric forms, all diastereomers, enantiomers and/or conformers of the basic molecular structure. Some compounds of the present disclosure can exist in different tautomeric forms, all of the latter being included within the scope of the present disclosure.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of treatment.

Substantially: As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

Suffering from: An individual who is “suffering from” a disease, disorder, and/or condition has been diagnosed with or displays one or more signs and symptoms of the disease, disorder, and/or condition.

Susceptible to: An individual who is “susceptible to” a disease, disorder, and/or condition has not been diagnosed with and/or cannot exhibit signs and symptoms of the disease, disorder, and/or condition but harbors a propensity to develop a disease or its signs and symptoms. In some embodiments, an individual who is susceptible to a disease, disorder, and/or condition (for example, an infectious respiratory disease) can be characterized by, for example, exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or other molecules of the present disclosure can be chemical or enzymatic.

Therapeutic Agent: The term “therapeutic agent” refers to an agent that, when administered to a subject, has a therapeutic, diagnostic, and/or prophylactic effect and/or elicits a desired biological and/or pharmacological effect. For example, in some embodiments, an mRNA encoding an antigen can be a therapeutic agent. In some embodiments, the therapeutic agent is not cystic fibrosis transmembrane conductance regulator (CFTR).

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve signs and symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve signs and symptoms of, diagnose, prevent, and/or delay the onset of the infection, disease, disorder, and/or condition.

Total daily dose: As used herein, a “total daily dose” is an amount given or prescribed in 24 hour period. The total daily dose can be administered as a single unit dose or a split dose.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms).

The notation “C₁₋₁₄ alkyl” means a linear or branched, saturated hydrocarbon including 1-14 carbon atoms. An alkyl group can be optionally substituted.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond.

The notation “C₂₋₁₄ alkenyl” means a linear or branched hydrocarbon including 2-14 carbon atoms and at least one double bond. An alkenyl group can include one, two, three, four, or more double bonds. An alkenyl group can be optionally substituted.

As used herein, the term “carbocycle” or “carbocyclic group” means a mono- or multi-cyclic system including one or more rings of carbon atoms. Rings can be three, four, five, six, seven, eight, nine, ten, eleven, twelve, thirteen, fourteen, or fifteen membered rings.

The notation “C₃₋₆ carbocycle” means a carbocycle including a single ring having 3-6 carbon atoms. Carbocycles can include one or more double bonds and can be aromatic (e.g., aryl groups). Examples of carbocycles include cyclopropyl, cyclopentyl, cyclohexyl, phenyl, naphthyl, and 1,2-dihydronaphthyl groups. Carbocycles can be optionally substituted.

As used herein, the term “heterocycle” or “heterocyclic group” means a mono- or multi-cyclic system including one or more rings, where at least one ring includes at least one heteroatom. Heteroatoms can be, for example, nitrogen, oxygen, or sulfur atoms. Rings can be three, four, five, six, seven, eight, nine, ten, eleven, or twelve membered rings. Heterocycles can include one or more double bonds and can be aromatic (e.g., heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. Heterocycles can be optionally substituted.

As used herein, an “aryl group” is a carbocyclic group including one or more aromatic rings. Examples of aryl groups include phenyl and naphthyl groups.

As used herein, a “heteroaryl group” is a heterocyclic group including one or more aromatic rings. Examples of heteroaryl groups include pyrrolyl, furyl, thiophenyl, imidazolyl, oxazolyl, and thiazolyl. Both aryl and heteroaryl groups can be optionally substituted.

Alkyl, alkenyl, and cyclyl (e.g., carbocyclyl and heterocyclyl) groups can be optionally substituted unless otherwise specified. Optional substituents can be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR or OC(O)R), an aldehyde (e.g., —C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)₂R″″, in which each OR are alkoxy groups that can be the same or different and R″″ is an alkyl or alkenyl group), a phosphate (e.g., P(O)₄₃), a thiol (e.g., SH), a sulfoxide (e.g., S(O)R), a sulfinic acid (e.g., S(O)OH), a sulfonic acid (e.g., S(O)₂OH), a thial (e.g., C(S)H), a sulfate (e.g., S(O)₄²), a sulfonyl (e.g., S(O)₂), an amide (e.g., C(O)NR², or N(R)C(O)R), an azido (e.g., N₃), a nitro (e.g., NO₂), a cyano (e.g., CN), an isocyano (e.g., NC), an acyloxy (e.g., OC(O)R), an amino (e.g., NR², NRH, or NH₂), a carbamoyl (e.g., OC(O)NR², OC(O)NRH, or OC(O)NH₂), a sulfonamide (e.g., S(O)₂NR², S(O)₂NRH, S(O)₂NH₂, N(R)S(O)₂R, N(H)S(O)₂R, N(R)S(O)₂H, or N(H)S(O)₂H), an alkyl group, an alkenyl group, and a cyclyl (e.g., carbocyclyl or heterocyclyl) group. R is an alkyl or alkenyl group, as defined herein.

As used herein, “comprises one to five primary, secondary, or tertiary amines or combination thereof” refers to alkyl, heterocycloalkyl, cycloalkyl, aryl, or heteroaryl groups that comprise, in addition to the other atoms, at least one nitrogen atom. The nitrogen atom is part of a primary, secondary, or tertiary amine group. The amine group can be selected from, but not limited to,

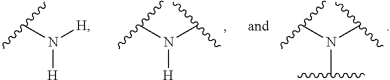

The primary, secondary, or tertiary amine can be part of a larger amine containing functional group selected from, but not limited to, —C(═N—)—N—, —C═C—N—, —C═N—, and —N—C(═N—)—N—.

Those skilled in the art will recognize, or be able to ascertain using no more than routine experimentation, many equivalents to the specific embodiments in accordance with the present disclosure described herein. The scope of the present disclosure is not intended to be limited to the above Description, but rather is as set forth in the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any embodiment of the compositions of the present disclosure (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### Additional Embodiments

1. A composition, comprising
   a polynucleotide payload and a nanoparticle, wherein the nanoparticle comprises a lipid nanoparticle core comprising an ionizable lipid, a phospholipid, a structural lipid, and a PEG-lipid, and a cationic agent dispersed primarily on the outer surface of the core.
2. An mRNA vaccine, comprising
   an mRNA comprising an open reading frame encoding an antigen, optionally an infectious disease antigen, optionally a viral antigen, and a nanoparticle, wherein the nanoparticle comprises a lipid nanoparticle core comprising an ionizable lipid, a phospholipid, a structural lipid, a PEG-lipid, and the mRNA, and a cationic agent dispersed primarily on the outer surface of the core.
3. An mRNA therapeutic, comprising
   an mRNA comprising an open reading frame encoding a therapeutic protein, wherein the therapeutic protein is not a lung protein and a nanoparticle, wherein the nanoparticle comprises a lipid nanoparticle core comprising the mRNA and a cationic agent dispersed primarily on the outer surface of the core.
4. The composition, mRNA vaccine or mRNA therapeutic of any one of paragraphs 1-3, wherein the polynucleotide or mRNA is encapsulated within the core.
5. The composition, mRNA vaccine or mRNA therapeutic of any one of paragraphs 1-3, wherein the nanoparticle has a greater than neutral zeta potential at physiological pH.
6. The composition, mRNA vaccine or mRNA therapeutic of any one of paragraphs 1-5, wherein a weight ratio of the cationic agent to nucleic acid vaccine is about 1:1 to about 4:1, about 1.25:1 to about 3.75:1, about 1.25:1, about 2.5:1, or about 3.75:1.
7. The composition, mRNA vaccine or mRNA therapeutic of any one of paragraphs 1-5, wherein the nanoparticle has a zeta potential of about 5 mV to about 20 mV, about 5 mV to about 20 mV, about 5 mV to about 15 mV, or about 5 mV to about 10 mV.
8. The composition, mRNA vaccine or mRNA therapeutic of any one of paragraphs 1-7, wherein greater than about 80%, greater than 90%, greater than 95%, or greater than 95% of the cationic agent is on the surface on the nanoparticle.
9. The composition, mRNA vaccine or mRNA therapeutic of any one of paragraphs 1-8, wherein at least about 50%, at least about 75%, at least about 90%, or at least about 95% of the mRNA is encapsulated within the core.
10. The composition, mRNA vaccine or mRNA therapeutic of any one of paragraphs 1-9, wherein a general polarization of laurdan (GPL) of the nanoparticle is greater than or equal to about 0.6.
11. The composition, mRNA vaccine or mRNA therapeutic of any one of paragraphs 1-10, wherein the nanoparticle has a d-spacing of greater than about 6 nm or greater than about 7 nm.
12. The composition, mRNA vaccine or mRNA therapeutic of any one of paragraphs 1-11, wherein at least 50%, at least 75%, at least 90%, or at least 95% of the nanoparticles have a surface fluidity value of greater than a threshold polarization level.
13. The composition, mRNA vaccine or mRNA therapeutic of any one of paragraphs 1-12, wherein about 10% or greater, about 15% or greater, or about 20% or greater of cell population has accumulated the nanoparticle when the nanoparticle is contacted with a population of mucosal cells.
14. The composition, mRNA vaccine or mRNA therapeutic of any one of paragraphs 1-13, wherein the cationic agent has a solubility of greater than about 1 mg/mL, greater than about 5 mg/mL, greater than about 10 mg/mL, or greater than about 20 mg/mL in alcohol.
15. The composition, mRNA vaccine or mRNA therapeutic of any one of paragraphs 1-14, wherein the cationic agent is a cationic lipid and the cationic lipid is a water-soluble amphiphilic molecule.
16. The composition, mRNA vaccine or mRNA therapeutic of paragraph 15, wherein the amphiphilic molecule comprises a lipid moiety and a hydrophilic moiety.
17. The composition, mRNA vaccine or mRNA therapeutic of any one of paragraphs 1-14, wherein the cationic agent is a cationic lipid and the cationic lipid comprises a structural lipid, fatty acid, or hydrocarbyl group.
18. The composition, mRNA vaccine or mRNA therapeutic of any one of paragraphs 1-14, wherein the cationic agent is a cationic lipid and the cationic lipid is a sterol amine comprising a hydrophobic moiety and a hydrophilic moiety.
19. The composition, mRNA vaccine or mRNA therapeutic of paragraph 18, wherein the hydrophilic moiety comprises an amine group comprising one to four primary, secondary, or tertiary amines or mixtures thereof.
20. The composition, mRNA vaccine or mRNA therapeutic of paragraph 19, wherein the amine group comprises one or two terminal primary amines.
21. The composition, mRNA vaccine or mRNA therapeutic of paragraph 19, wherein the amine group comprises one or two terminal primary amines and one internal secondary amine.
22. The composition, mRNA vaccine or mRNA therapeutic of paragraph 19, wherein the amine group comprises one or two tertiary amines.
23. The composition, mRNA vaccine or mRNA therapeutic of any one of paragraphs 19-22, wherein the amine group has a pKa value of greater than about 8.
24. The composition, mRNA vaccine or mRNA therapeutic of any one of paragraphs 19-22, wherein the amine group has a pKa value of greater than about 9.
25. The composition, mRNA vaccine or mRNA therapeutic of any one of paragraphs 18-24, wherein the sterol amine is a compound of Formula (A1):

A-L-B (A1)
or a salt thereof, wherein:
A is an amine group, L is an optional linker, and B is a sterol.
26. The composition, mRNA vaccine or mRNA therapeutic of any one of paragraphs 18-25, wherein the sterol amine has Formula A2a:

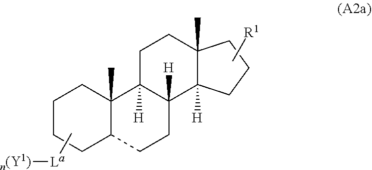

or a salt thereof, wherein:
   [symbol] is a single or double bond
   R¹ is C₁₋₁₄ alkyl or C₁₋₁₄ alkenyl;
   L^{a} is absent, —O—, —S—S—, —OC(═O), —C(═O)N—, —OC(═O)N—, CH₂—NH—C(O)—, —C(═O)O—, —OC(═O)—CH₂—CH₂—C(═O)N—, —S—S—CH₂, —SS—CH₂—CH₂—C(═O)N—, or a group of formula (a):

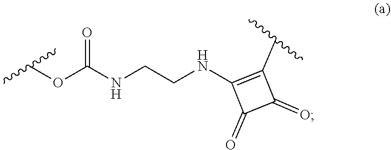 [SMILES: *OC(=O)NCCNc1c(*)c(=O)c1=O]

Y¹ is C₁₋₁₀ alkyl, 3 to 8-membered heterocycloalkyl, 5 to 6-membered heteroaryl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) are each optionally substituted with 1, 2, 3, or 4 substituents independently selected from C₁₋₆ alkyl, halo, OH, —O(C₁₋₆ alkyl), —C₁₋₆ alkyl-OH, NH₂, —NH(C₁₋₆ alkyl), N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH-(3 to 8-membered heterocycloalkyl), and —NH(5 to 6-membered heteroaryl); and
n is 1 or 2, and
optionally:
   wherein [symbol] is a double bond
   wherein [symbol] is a single bond,
   wherein L^{a} is —OC(═O), —OC(═O)N—, or —OC(═O)—CH₂—CH₂—C(═O)N—,
   wherein n is 1,
   wherein n is 2,
   wherein R¹ is C₁₋₁₄ alkyl,
   wherein R¹ is C₁₋₁₄ alkenyl,
   wherein R¹ is

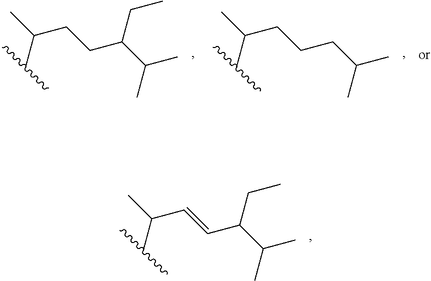

and/or
wherein Y¹ is C₁₋₁₀ alkyl, 3 to 8-membered heterocycloalkyl, —C₁₋₆ alkyl-(3 to 8-membered heterocycloalkyl), or —C₁₋₆ alkyl-(5 to 6-membered heteroaryl), wherein the C₁₋₁₀ alkyl, 3 to 8-membered heterocycloalkyl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof; and wherein the C₁₋₁₀ alkyl, C₁₋₆ alkyl-(3 to 8-membered heterocycloalkyl), and C₁₋₆ alkyl-(5 to 6-membered heteroaryl) are each optionally substituted with C₁₋₆ alkyl, OH, —C₁₆ alkyl-OH, or NH₂.
27. The composition, mRNA vaccine or mRNA therapeutic of paragraph 26, wherein Y¹ is selected from:
   (1)

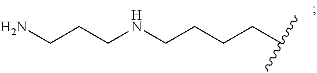 [SMILES: *CCCCNCCCN]

(2)

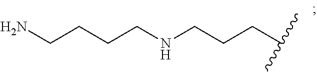 [SMILES: *CCCNCCCCN]

(3)

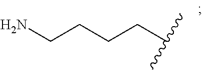 [SMILES: *CCCCN]

(4)

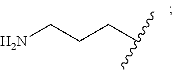 [SMILES: *CCCN]

(5)

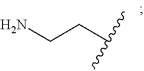 [SMILES: *CCN]

(6)

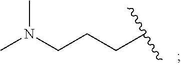 [SMILES: *CCCN(C)C]

(7)

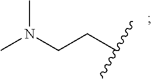 [SMILES: *CCN(C)C]

(8)

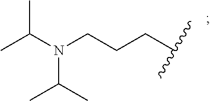 [SMILES: *CCCN(C(C)C)C(C)C]

(9)

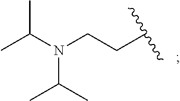 [SMILES: *CCN(C(C)C)C(C)C]

(10)

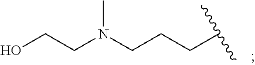 [SMILES: *CCCN(C)CCO]

(11)

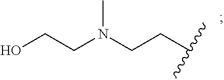 [SMILES: *CCN(C)CCO]

(12)

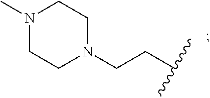 [SMILES: *CCN1CCN(C)CC1]

(13)

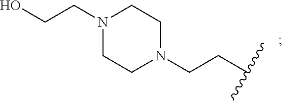 [SMILES: *CCN1CCN(CCO)CC1]

(14)

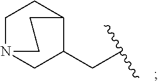 [SMILES: *CC1CN2CCC1CC2]

(15)

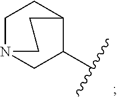 [SMILES: *C1CN2CCC1CC2]

(16)

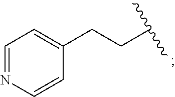 [SMILES: *CCc1ccncc1]

(17)

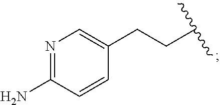 [SMILES: *CCc1ccc(N)nc1]

(18)

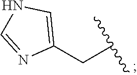 [SMILES: *Cc1c[nH]cn1]

(19)

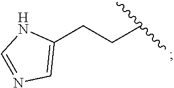 [SMILES: *CCc1cnc[nH]1]

(20)

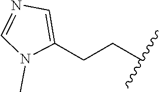 [SMILES: *CCc1cncn1C]

(21)

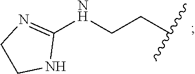 [SMILES: *CCNC1=NCCN1]

(22)

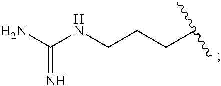 [SMILES: *CCCNC(=N)N]

(23)

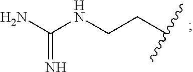 [SMILES: *CCNC(=N)N]

(28) N(CH₃)₂; (29)

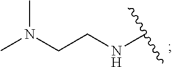 [SMILES: *NCCN(C)C]

(30)

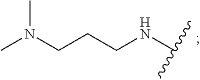 [SMILES: *NCCCN(C)C]

(31)

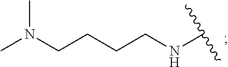 [SMILES: *NCCCCN(C)C]

and (32)

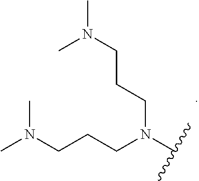 [SMILES: *N(CCCN(C)C)CCCN(C)C]

28. The composition, mRNA vaccine or mRNA therapeutic of any one of paragraphs 18-25, wherein the sterol amine has Formula A4

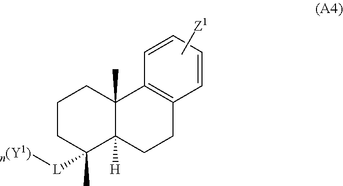

or a salt thereof, wherein:
   Z¹ is OH or C₃₋₆ alkyl;
   L is absent, —O—, —S—S—, —OC(═O), —C(═O)N—, —OC(═O)N—, —CH₂—NH—C(═O)—, —C(═O)O—, —OC(═O)—CH₂—CH₂—C(═O)N—, —S—S—CH₂, or —SS—CH₂—CH₂—C(O)N—;
Y¹ is C₁₋₁₀ alkyl, 3 to 8-membered heterocycloalkyl, 5 to 6-membered heteroaryl, —C₁₋₆ alkyl-(3 to 8 membered heterocycloalkyl), or —C₁₋₆ alkyl-(5 to 6 membered heteroaryl),
   wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
   and wherein the C₁₋₁₀ alkyl, 3 to 8-membered heterocycloalkyl, 5 to 6 membered heteroaryl, —C₁₋₆ alkyl-(3 to 8-membered heterocycloalkyl), and —C₁₋₆ alkyl-(5 to 6-membered heteroaryl) are each optionally substituted with 1, 2, 3, or 4 substituents selected from C₁₋₆ alkyl, halo, OH, —O(C₁₋₆ alkyl), —C₁₋₆ alkyl-OH, NH₂, —NH(C₁₋₆ alkyl), —N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH(3 to 8-membered heterocycloalkyl), and —NH(5 to 6-membered heteroaryl); and
   n is 1 or 2, and
optionally:
   wherein Z¹ is OH,
   wherein Z¹ is C₃₋₆ alkyl,
   wherein L is —C(═O)N—, —CH₂—NH—C(═O)—, or —C(═O)O—,
   wherein Y¹ is C₁₋₁₀ alkyl comprising one to five primary, secondary, or tertiary amines or combination thereof,
   wherein Y¹ is

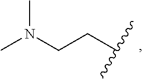

wherein n is 1, and/or
wherein n is 2.
29. The composition, mRNA vaccine or mRNA therapeutic of paragraph 18, wherein the sterol amine is selected from:
   (a) SA1, SA2, SA3, SA4, SA5, SA6, SA7, SA8, SA9, SA10, SA11, SA12, SA13, SA14, SA15, SA16, SA17, SA18, SA19, SA20, SA21, SA22, SA23, SA24, SA25, SA26, SA27, SA28, SA29, SA30, SA31, SA32, SA33, SA34, SA35, SA36, SA37, SA38, SA39, SA40, SA41, SA42, SA43, SA44, SA45, SA46, SA47, SA48, SA49, SA50, SA51, SA52, SA53, SA54, SA55, SA56, SA57, SA58, SA59, SA60, SA61, SA62, SA63, SA64, SA65, SA66, SA67, SA68, SA69, SA70, SA71, SA72, SA73, SA74, SA75, SA76, SA77, SA78, SA79, SA81, SA82, SA83, SA84, SA85, SA86, SA87, SA88, SA89, SA90, SA91, SA92, SA93, SA94, SA95, SA96, SA97, SA98, SA110, SA111, SA113, SA114, SA116, SA117, SA118, SA119, SA120, SA121, SA122, SA123, SA124, SA125, SA126, SA127, SA128, SA129, SA130, SA131, SA132, SA133, SA134, SA135, SA136, SA137, SA138, SA139, SA141, SA142, SA144, SA145, SA149, SA151, SA152, SA153, SA154, SA155, SA156, SA157, SA158, SA159, SA160, SA161, SA162, SA163, SA164, SA165, SA166, SA167, SA168, SA169, SA170, SA171, SA172, SA173, SA174, SA175, SA176, SA177, SA178, SA179, SA180, SA181, SA182, SA183, SA184, and SA185; or
   (b) SA186, SA187, SA188, and SA189.
30. The composition, mRNA vaccine or mRNA therapeutic of any one of paragraphs 1-29, wherein cationic agent is a non-lipid cationic agent.
31. The composition, mRNA vaccine or mRNA therapeutic of paragraph 30, wherein the non-lipid cationic agent is benzalkonium chloride, cetylpyridium chloride, L-lysine monohydrate, or tromethamine.
32. The composition, mRNA vaccine or mRNA therapeutic of any one of paragraphs 1-25, wherein the cationic agent is a modified arginine.
33. The composition, mRNA vaccine or mRNA therapeutic of any one of paragraphs 1-32, wherein the nanoparticle comprises about 30 mol % to about 60 mol % or about 40 mol % to about 50 mol % of ionizable lipid.
34. The composition of any one of paragraphs 1-33 wherein the ionizable lipid is compound 18:

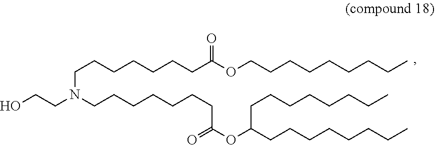 [SMILES: CCCCCCCCCOC(=O)CCCCCCCN(CCO)CCCCCCCC(=O)OC(CCCCCCCC)CCCCCCCC]

or a salt thereof.
35. The composition, mRNA vaccine or mRNA therapeutic of any one of paragraphs 1-34, wherein the nanoparticle comprises about 5 mol % to about 15 mol %, about 8 mol % to about 13 mol %, or about 10 mol % to about 12 mol % of phospholipid.
36. The composition, mRNA vaccine or mRNA therapeutic of any one of paragraphs 1-35, wherein the phospholipid is 1,2-distearoyl sn glycerol 3-phosphocholine (DSPC).
37. The composition, mRNA vaccine or mRNA therapeutic of any one of paragraphs 1-36, wherein the nanoparticle comprises about 20 mol % to about 60 mol %, about 30 mol % to about 50 mol %, about 35 mol %, or about 40 mol % structural lipid.
38. The composition, mRNA vaccine or mRNA therapeutic of paragraph 36 or 37, wherein the mRNA is administered by mucosal administration, intranasal or intrabronchial administration.
39. The composition, mRNA vaccine or mRNA therapeutic of paragraph 38, wherein the mRNA vaccine or composition is administered by nebulizer or inhaler or droplet.
40. The composition, mRNA vaccine or mRNA therapeutic of paragraph 1, wherein the polynucleotide payload comprises an mRNA encoding a polypeptide, wherein the polypeptide does not comprise a cystic fibrosis transmembrane conductance regulator (CFTR) protein.
41. A method, comprising
   administering to a mucosal surface of a subject a composition comprising a polynucleotide payload and a nanoparticle, wherein the nanoparticle comprises a lipid nanoparticle core comprising an ionizable lipid, a phospholipid, a structural lipid, and a PEG-lipid, and a cationic agent dispersed primarily on the outer surface of the core.
42. The method of paragraph 41, wherein the polynucleotide or mRNA is encapsulated within the core.
43. The method of paragraph 41 or 42, wherein the nanoparticle has a greater than neutral zeta potential at physiological pH.
44. The method of any one of paragraphs 41-43, wherein a weight ratio of the cationic agent to nucleic acid vaccine is about 1:1 to about 4:1, about 1.25:1 to about 3.75:1, about 1.25:1, about 2.5:1, or about 3.75:1.
45. The method of any one of paragraphs 41-43, wherein the polynucleotide payload is a mRNA.
46. The method of paragraph 45, wherein the mRNA is an mRNA encoding an antigen and wherein the composition is administered in an effective amount to induce an immune response to the antigen.
47. The method of paragraph 46, wherein the antigen is an infectious disease antigen.
48. The method of paragraph 45, wherein the mRNA is an mRNA encoding a therapeutic protein.
49. The method of any one of paragraph 41-48, wherein the mucosal surface comprises a cell population selected from respiratory mucosal cells, oral mucosal cells, intestinal mucosal cells, vaginal mucosal cells, rectal mucosal cells, and buccal mucosal cells.
50. The composition, mRNA vaccine or mRNA therapeutic of any one of paragraphs 18-25, wherein the sterol amine has Formula A6:

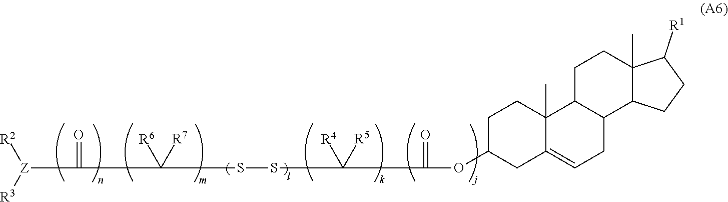

or a salt thereof, wherein:
   Z is N or CH;
   R¹ is C₁₋₁₄ alkyl, C₁₋₁₄ alkenyl, or C₁₋₁₄ hydroxyalkyl;
   R² and R³ are each C₂₋₂₀ alkyl, wherein:
   (i) the C₂₋₂₀ alkyl is substituted by 1, 2, 3, 4, or 5 substituents independently selected from —NR⁸R⁹, OH, and halo, wherein at least one substituent is —NR⁸R⁹;
   (ii) 1, 2, 3, or 4 non-terminal carbons of the C₂₋₂₀ alkyl are optionally replaced with 0;
   (iii) 1, 2, 3, or 4 non-terminal carbons of the C₂₋₂₀ alkyl are optionally replaced with NR¹⁰;
   (iv) 1, 2, 3, or 4 non-terminal carbons of the C₂₋₂₀ alkyl are optionally replaced with C(═O); and
   (v) 1, 2, 3, or 4 non-terminal carbons of the C₂₋₂₀ alkyl are optionally replaced with CR^{a}R^{b} wherein R^{a} and R^{b} together with the C atom to which they are attached form a C₃₋₆ cycloalkyl group;
   wherein R² and R³ are the same or different;
   or R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR⁸R⁹, OH, and halo;
   or R², R³, and R⁶, together with the atoms to which they are attached and any intervening atoms, form a 7-18 membered bridged heterocycloalkyl group optionally substituted with 1, 2, or 3 substituents independently selected from C₁₋₄ alkyl, —NR⁸R⁹, OH, and halo;
   R⁴, R⁵, R⁶, and R⁷ are each independently selected from H, halo, and C₁₋₄ alkyl;
   or R⁴ and R⁵ together with the carbon atom to which they are attached form a C₃_a cycloalkyl group;
   or R⁶ and R⁷ together with the carbon atom to which they are attached form a C₃_a cycloalkyl group;
   R⁸, R⁹, and R¹⁰ are each independently selected from H and C₁₋₄ alkyl;
   j is 0 or 1;
   k is 0, 1, 2, 3, 4, 5, or 6;
   l is 0 or 1;
   m is 0, 1, 2, 3, 4, 5, or 6; and
   n is 0 or 1;
   wherein when j is 0, then l is 1,
   wherein j and 1 are not both 0.
51. The composition, mRNA vaccine or mRNA therapeutic of any one of paragraphs 18-25, wherein the sterol amine has Formula A8:

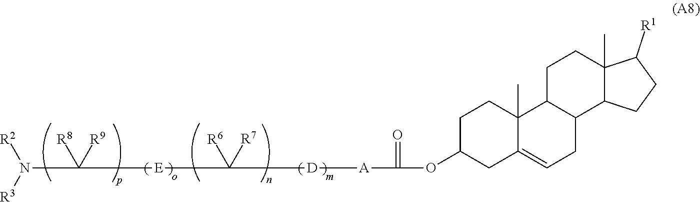

or a salt thereof, wherein:
   A is NR^{a} or CR⁴R⁵;
   D is O or S—S;
   E is C(O), C(O)NH, or 0;
   R¹ is C₁₋₁₄ alkyl, C₁₋₁₄ alkenyl, or C₁₋₁₄ hydroxyalkyl;
   R² and R³ are each independently selected from H, methyl, and ethyl, wherein the methyl or ethyl is optionally substituted by OH;
   or R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR⁸R⁹, OH, and halo;
   or R², R³, and R⁸, together with the atoms to which they are attached and any intervening atoms, form a 7-18 membered bridged heterocycloalkyl group optionally substituted with 1, 2, or 3 substituents independently selected from C₁₋₄ alkyl, —NR⁸R⁹, OH, and halo;
   R^{a} is H or methyl;
   R⁴, R⁵, R⁶, R⁷, R¹, R⁹, and R¹⁰ are each independently selected from H and C₁₋₄ alkyl;
   or R⁴ and R⁵ together with the carbon atom to which they are attached form a C₃₋₅ cycloalkyl group; or R⁶ and R⁷ together with the carbon atom to which they are attached form a C₃₋₅ cycloalkyl group; or R⁸ and R⁹ together with the carbon atom to which they are attached form a C₃₋₅ cycloalkyl group; m is 0 or 1;
   n is 0, 1, 2, 3, 4, or 5;
   is 0 or 1; and
   p is 0, 1, 2, 3, 4, 5, 6, 7, 8, 9, 10, 11, or 12;
   wherein at least one of m, n, o, and p is other than 0;
   wherein p is 1 when R², R³, and R⁸, together with the atoms to which they are attached and any intervening atoms, form a 7-18 membered bridged heterocycloalkyl group optionally substituted with 1, 2, or 3 substituents independently selected from C₁₋₄ alkyl, —NR⁸R⁹, OH, and halo; and
   wherein when m is 1, then A is CR⁴R⁵ and n is 1.

### EXAMPLES

### Abbreviations

ACN: Acetonitrile
Aq.: Aqueous
Boc₂O: Di-tert-butyl pyrocarbonate
DBU: 1,8-Diazabicyclo[5.4.0]undec-7-ene
DCC: N,N′-Dicyclohexylcarbodiimide
DCM: Dichloromethane
DMAP: 4-Dimethylaminopyridine
DMF: Dimethylformamide
EtOAc: Ethyl acetate
Hrs: hours
h: hour/hours
LCMS: Liquid chromatography-mass spectrometry
MTBE: Methyl tert-butyl ether
PMA: Phosphomolybdic Acid
rt: room temperature
THF: Tetrahydrofuran
TLC: Thin layer chromatography

### Example 1

### Production of Nanoparticle Compositions

Lipid nanoparticle cores were prepared using ethanol drop nanoprecipitation followed by solvent exchange into an aqueous buffer using a desalting chromatography column. An exemplary lipid nanoparticle can be prepared by a process where lipids were dissolved in ethanol at concentration of 15.4 mM and molar ratios of 50:10:38.5:1.5 (ionizable lipid:DSPC:cholesterol:DMG-PEG2K lipid) and mixed with mRNA at a concentration of 0.1515 mg/mL diluted in 25 mM sodium acetate pH 5.0. The N:P ratio was set to 5.8 in each formulation. The lipid solution and mRNA were mixed using a micro-tee mixer at a 1:3 volumetric ratio of lipid:mRNA. Once the nanoparticles were formed, they underwent solvent exchange over a desalting chromatography column preconditioned with 1×PBS buffer at pH 7.0. The elution profile of the nanoparticle was captured by UV, pH, and conductivity detectors. The UV profile was used to collect the solvent-exchanged nanoparticles. The resulting nanoparticle suspension underwent concentration using Amicon ultra-centrifugal filters and was passed through a 0.22 μm syringe filter. The nanoparticles were prepared to a specific concentration.

SA3 was added to the nanoparticle core by dissolving SA3 in macrogol (15)-hydroxy stearate, Kolliphor® HS15 (HS15) and post-added to LNP at a mass ratio of 1.25 (SA3 to mRNA). Specifically, 3HCl-SA3 was dissolved directly in HS15 (1 mg/mL, ˜70 μM, water) to generate initial stock solution at 5 mg/mL (6.92 mM), which could be in micellar form in solution. SA3 at 5 mg/mL was further diluted ([SA3] required for post-addition (PA) at a specific SA3:mRNA weight ratio) with HS15 (1 mg/mL) and added to LNPs (1:1 by volume) at ambient temperature via simple mixing:
[mRNA]0.2 mg/mL, [3HCl-SA3] 0.25 mg/mL, [HS15] 0.5 mg/mL, [PBS] 0.5×.
LNPs further diluted with 1×PBS (1:1 by volume):
[mRNA] 0.1 mg/mL, [3HCl-SA3] 0.125 mg/mL, [HS15] 0.5 mg/mL, [PBS] 0.75×.

An example LNP core, designated LNP-1a is as follow:

TABLE 7
LNP-1a

| | mass | MW | Core LNP |
|---|---|---|---|
| Components | (mg) | (g/mol) | mol % |
| Compound 18 | 11.99 | 710.18 | 50.0% |
| DSPC | 2.67 | 790.15 | 10.0% |
| Cholesterol | 5.02 | 386.65 | 38.5% |
| DMG-PEG 2k | 1.27 | 2500 | 1.5% |
| mRNA | 1 | — | — |

An example LNP as described, designated LNP-1 is as follows:

TABLE 8
LNP-1

| | mass | MW | Core LNP | LNP-1 | LNP-1 |
|---|---|---|---|---|---|
| Components | (mg) | (g/mol) | mol % | mol % | mass % |
| Compound 18 | 11.99 | 710.18 | 50.0% | 47.6% | 51.7% |
| DSPC | 2.67 | 790.15 | 10.0% | 9.5% | 11.5% |
| Cholesterol | 5.02 | 386.65 | 38.5% | 36.6% | 21.6% |
| DMG-PEG 2k | 1.27 | 2500 | 1.5% | 1.4% | 5.5% |
| SA3•3HCl | 1.25 | 724 | — | 4.9% | 5.4% |
| HS 15 | 0.25 | — | — | — | — |
| mRNA | 1 | — | — | — | 4.3% |

HS15 has a MW of 960-1900, with average MW of 1430.

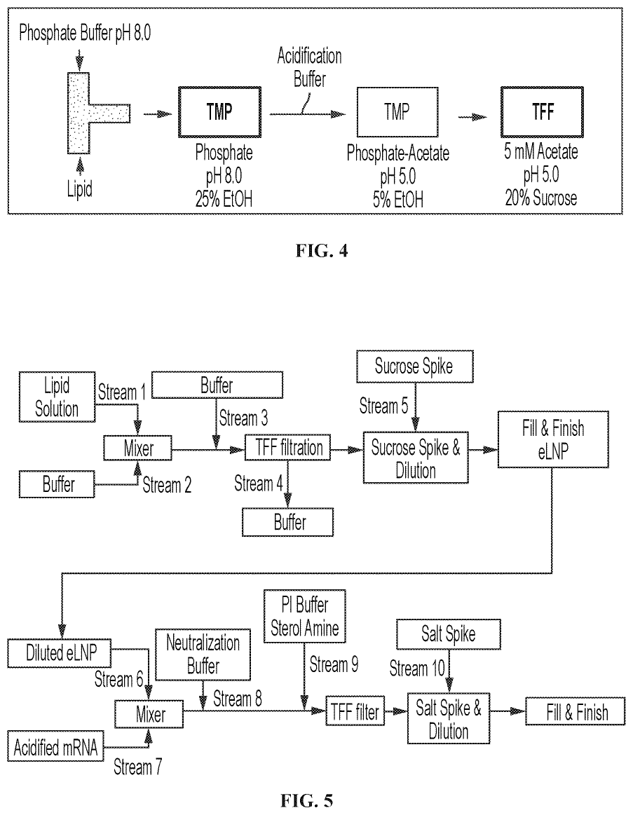

Exemplary LNP (without SA3) can be prepared according to the schematic in FIGS. 1-3. FIG. 1 refers to post-hoc loading (PHL) process of generating an empty lipid nanoparticle and the solution containing nucleic acid is then added to an empty-LNP. FIG. 2 refers to post-insertion/post-addition (PHL-PIPA) process refers to adding PEG lipid to a lipid nanoparticle. FIG. 3 refers to second generation post-hoc loading process, which includes post-insertion/post-addition of PEG steps. FIG. 4 refers to empty lipid nanoparticle prototype (“Neutral assembly”), where the empty LNP is mixed at pH 8.0 and the final formulation is pH 5.0.

### Example 2

### Percent mRNA Encapsulation

Encapsulation efficiency (EE %) was measured using a modified Quant-iT RiboGreen assay. To determine the EE %, nanoparticles (or PBS, blank) were diluted in 1× TE to achieve a concentration of 2-4 μg/mL mRNA per well. These samples were aliquoted and diluted 1:1 in 1×TE or 1×TE with 2.5 mg/mL heparin buffer (measuring free mRNA) or TE buffer with 2% Triton X-100 or 2% Triton with 2.5 mg/mL heparin (measuring total mRNA). Quant-iT RiBogreen reagent was added and fluorescent signal was quantified using a plate reader. Encapsulation efficiency was calculated as follows:

$$EE \% = ( 1 - \frac{Free mRNA}{Total mRNA} ) * 100$$

Total mRNA: quantification of the total amount of mRNA by dissolving the particles with the detergent Triton (TX) with or without heparin.
Free mRNA: quantification of the amount of mRNA that is not encapsulated by diluting the particles in TE (Tris+EDTA buffer) with or without heparin.
Heparin is an anionic glycosaminoglycan, which competes with the sterol amine for the mRNA, and is used to quantify the amount of mRNA in LNP with a cationic agent such as sterol amine.
LNP-1 prepared according to Example 1 has 98% encapsulated mRNA.

### Example 3

### LNP Cellular Uptake and Protein Expression in Healthy Human Bronchial Epithelial Cell Models

To evaluate LNP cellular uptake and protein expression in healthy human bronchial epithelial cells (HBE), the EpiAirway model from MatTek (Ashland, MA) a ready-to-use 3D tissue model is used. The model consists of human-derived tracheal/bronchial epithelial cells from healthy donors.

The cells are plated on 24 mm transwells inserts with a pore size of 0.4 μm, and upon developing a confluent monolayer, media is removed from the apical chamber, with cultures being kept at the air-liquid interface (ALI) for up to 4 weeks to achieve complete cell differentiation and pseudo-stratification. The model recapitulates in vivo phenotypes of mucociliary barriers and exhibits human relevant tissue structure and cellular morphology, with a 3D structure consisting of organized Keratin 5+ basal cells, mucus producing goblet cells, functional tight junctions and beating cilia.

### Accumulation and Expression Assay in Healthy HBE Cells

LNPs incorporating 0.1 mole % Rhodamine-DOPE and encapsulating NPI-Luc reporter mRNA were dosed apically in healthy HBE in Hyclone Phosphate Buffered Saline. The cells were washed with 1 mM DTT in PBS for 10 min prior to LNP addition to remove the mucus accumulated during post-ALI differentiation. The NPI-Luc reporter includes a nuclear localization sequence and multiple V5 tags at N-terminus for enhanced detection sensitivity of expressed protein molecules. LNP transfected cells were incubated 4-72 h, after that the cells were detached from membranes using trypsin EDTA and fixed in suspension with 4% PFA in PBS.

Cells were processed separately for LNP accumulation and protein expression. To quantify LNP accumulation, PFA fixed cells were transferred in 96 well Cell Carrier Ultra plates (PerkinElmer) with optically-clear cyclic olefin bottom for high content analysis, and imaged using Opera Phenix spinning disk confocal microscope (PerkinElmer). Cells were detected using DAPI (405 nm channel), and LNP accumulation was detected using the Rhodamine-DOPE (561 nm channel). Image analysis was performed in Harmony 4.8, using spot segmentation in the 561 nm channel to quantify LNP accumulation in endocytic organelles, and to derive % cells positive for LNP uptake as wells as LNP accumulation per cell.

To quantify protein expression, PFA fixed cells were transferred in 96 well v-bottom plates and processed for immunofluorescence (IF) using an anti-V5 rabbit monoclonal antibody. Briefly, the cells were permeabilized with 0.5% TX-100 for 5 min, blocked with 1% bovine serum albumin (BSA) in PBS for 30 min, followed by incubation with anti-V5 primary antibody for 1 h at room temperature, and Alexa 488 conjugated secondary antibody for 30 min. Between the different incubation steps the cells were spun down and washed by resuspension in PBS. Following anti-V5 IF staining, the cells were transferred in 96 well Cell Carrier Ultra plates for imaging with the Opera Phenix, NPI-Luc expression was detected was using the 488 nm channel. Image analysis was performed in Harmony 4.8, with mean nuclear intensity in the 488 nm channel being used to derive % cells positive for protein expression and protein expression per cell.

### Example 4

### Protein Expression in Human Cervical Cancer Epithelial Cell (HeLa) Model

To evaluate protein expression In Vitro, HeLa cells from ATCC.org (ATCC CCL-2) are used. The cells are cultured in complete Minimum Essential Medium (MEM) and are plated in 96 well Cell Carrier Ultra plate with PDL coated surface (PerkinElmer) prior to running an experiment.

### Expression Assay in HeLa Cells

LNPs encapsulating NPI-Luc mRNA were dosed with MEM media in the absence of serum. LNP transfected cell were incubated for 5 h post LNP transfection, the cells were imaged live using Opera Phoenix spinning disk confocal microscope (PerkinElmer). Cells were detected using DAPI (405 nm channel), and image analysis was performed in Harmony 4.9, to quantify the number of cells. After imaging the cells were processed with One-Glo Luciferase assay (Promega) to quantify protein expression. Results were reported in relative luminescence units (RLU) normalized to cells counts.

### Example 5

### Production of Nanoparticle Compositions Using a Post-Hoc Approach

Exemplary empty lipid nanoparticles can be prepared by a process where lipids were dissolved in ethanol at concentration of 40 mM and molar ratios of 50.5:10.1:38.9:0.5 (ionizable lipid:DSPC:cholesterol:DMG-PEG2K lipid) and mixed with 7.15 mM sodium acetate pH 5.0. The lipid solution and buffer were mixed using a multi-inlet vortex mixer at a 3:7 volumetric ratio of lipid:buffer. After a 5 second residence time, the eLNPs were mixed with 5 mM sodium acetate pH 5.0 at a volumetric ratio of 5:7 of eLNP:buffer. The dilute eLNPs were then buffer exchanged and concentrated using tangential flow filtration into a final buffer containing 5 mM sodium acetate pH 5.0 and a sucrose solution was subsequently added to complete the storage matrix. mRNA loading into the eLNP took place using the PHL process. An exemplary mRNA-loaded nanoparticle can be prepared by mixing eLNP at a lipid concentration of 2.85 mg/mL with mRNA at a concentration of 0.25 mg/mL in 42.5 mM sodium acetate pH 5.0. The N:P ratio was set to 4.93 in each formulation. The eLNP solution and mRNA were mixed using a multi-inlet vortex mixer at a 3:2 volumetric ratio of eLNP:mRNA. Once the eLNP were loaded with mRNA, they underwent a 30 s-60 s residence time prior to mixing in-line with a buffer containing 120 mM TRIS pH 8.12 at a volumetric ratio of 5:1 of nanoparticle:buffer. After this addition step, the nanoparticle formulation was mixed in-line with a buffer containing 20 mM TRIS, 0.352 mg/mL DMG-PEG2k, 0.625 mg/mL SA3, pH 7.5 at a volumetric ratio of 6:1 of nanoparticle:buffer. The resulting nanoparticle suspension underwent concentration using tangential flow filtration and was diluted with a salt solution to a final buffer matrix containing 70 mM NaCl. The resulting nanoparticle suspension was filtered through a 0.8/0.2 μm capsule filter and filled into glass vials a mRNA strength of 0.5-2 mg/mL.

### Example 6

### Synthesis of Sterol Amines

The synthesis of sterol amines SA1-SA43 is described in WO 2022/032154, the entire contents of which are incorporated herein by reference in their entirety.

### A. Compound SA44: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-((2-hydroxyethyl)(methyl)amino)ethyl)carbamate

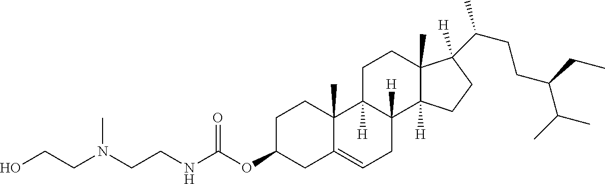

To a solution of β-sitosterol 4-nitrophenyl carbonate (0.120 g, 0.207 mmol) and triethylamine (0.04 mL, 0.3 mmol) in DCM (2.0 mL) was added a solution of 2-[(2-aminoethyl)(methyl)amino]ethanol (0.029 g, 0.25 mmol) in DCM (0.5 mL). The reaction mixture stirred at 40° C. and was monitored by LCMS. At 3 h, the reaction mixture was diluted with DCM and washed with water. The organics were passed through a hydrophobic frit, dried over Na₂SO₄, and concentrated. The crude material was purified via silica gel chromatography (0-12% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-((2-hydroxyethyl)(methyl)amino)ethyl)carbamate (0.072 g, 0.12 mmol, 58.5%) as a white foam. UPLC/ELSD: RT=2.30 min. MS (ES): m/z=559.6 [M+H]⁺ for C₃₅H₆₂N₂O₃; ¹H NMR (300 MHz, CDCl₃): δ 5.34-5.41 (m, 1H), 4.89 (br. s, 1H), 4.42-4.57 (m, 1H), 3.61 (t, 2H, J=5.4 Hz), 3.28 (dt, 2H, J=5.9, 5.6 Hz), 2.51-2.61 (m, 4H), 2.21-2.41 (m, 2H), 2.28 (s, 3H), 1.76-2.07 (br. m, 5H), 0.88-1.74 (br. m, 22H), 1.01 (s, 3H), 0.92 (d, 3H, J=6.5 Hz), 0.79-0.87 (m, 9H), 0.68 (s, 3H).

### B. Compound SA45: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-(quinuclidin-3-yl)acetate

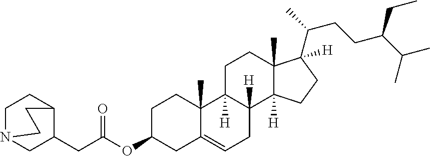

To a stirred solution of β-sitosterol (150 mg, 0.362 mmol), 3-(carboxymethyl)-1-azabicyclo[2.2.2]octan-1-ium chloride (AstaTech, Inc., Bristol, PA) (97 mg, 0.47 mmol), triethylamine (0.08 mL, 0.5 mmol), and 4-(dimethylamino)pyridine (0.022 g, 0.18 mmol) in DCM (2.4 mL) was added 1-ethyl-3-(3-dimethylaminopropyl)carbodiimide hydrochloride (0.104 g, 0.543 mmol). The reaction mixture stirred at rt and was monitored by LCMS. At 15 h, water (ca. 2.5 mL) was added, and the biphasic mixture stirred for 5 min. After this time, the layers were separated, and the aqueous was extracted with DCM (2×) and 9:1 DCM/MeOH. The combined organics were dried over Na₂SO₄ and concentrated. The crude material was purified via silica gel chromatography (0-20% (10% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-(quinuclidin-3-yl)acetate (0.106 g, 0.170 mmol, 47.1%) as a white solid. UPLC/ELSD: RT=2.57 min. MS (ES): m/z=566.6 [M+H]⁺ for C₃₈H₆₃NO₂; ¹H NMR (300 MHz, CDCl₃): δ 5.34-5.41 (m, 1H), 4.55-4.69 (m, 1H), 3.08-3.21 (m, 1H), 2.72-2.93 (m, 4H), 2.25-2.44 (br. m, 5H), 1.76-2.17 (br. m, 6H), 0.89-1.73 (br. m, 27H), 1.02 (s, 3H), 0.92 (d, 3H, J=6.5 Hz), 0.78-0.88 (m, 9H), 0.68 (s, 3H).

### C. Compound SA46: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-(dimethylamino)propyl)carbamate

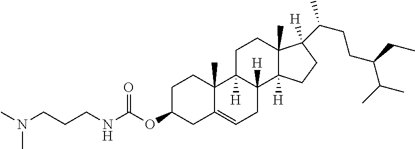

β-Sitosterol 4-nitrophenyl carbonate (0.150 g, 0.259 mmol), triethylamine (0.06 mL, 0.4 mmol), and dimethylaminopropylamine (0.04 mL, 0.3 mmol) were combined in CHCl₃ (2.4 mL) and stirred at 50° C. The reaction was monitored by TLC. At 21.5 hrs, the reaction mixture was cooled to rt and diluted with DCM (10 mL). The organics were washed with 5% aq. NaHCO₃ solution. The aqueous layer was extracted with DCM, and then the combined organics were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-(dimethylamino)propyl)carbamate (0.124 g, 0.218 mmol, 84.1%) as a white foam. UPLC/ELSD: RT=2.51 min. MS (ES): m/z=543.1 [M+H]⁺ for C₃₅H₆₂N₂O₂; ¹H NMR (300 MHz, CDCl₃): δ 5.28-5.40 (m, 2H), 4.42-4.56 (m, 1H), 3.23 (dt, 2H, J=6.4, 6.0 Hz), 2.17-2.42 (m, 4H), 2.22 (s, 6H), 1.77-2.05 (br. m, 5H), 0.88-1.72 (br. m, 24H), 1.01 (s, 3H), 0.92 (d, 3H, J=6.5 Hz), 0.77-0.88 (m, 9H), 0.68 (s, 3H).

### D. Compound SA47: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(dimethylamino)butyl)carbamate

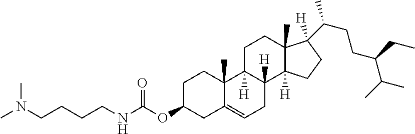

β-Sitosterol 4-nitrophenyl carbonate (0.150 g, 0.259 mmol), triethylamine (0.06 mL, 0.4 mmol), and (4-aminobutyl)dimethylamine (0.05 mL, 0.4 mmol) were combined in CHCl₃ (2.4 mL) and stirred at 50° C. The reaction was monitored by TLC. At 21.5 hrs, the reaction mixture was cooled to rt and diluted with DCM (10 mL). The organics were washed with 5% aq. NaHCO₃ solution. The aqueous layer was extracted with DCM, and then the combined organics were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(dimethylamino)butyl)carbamate (0.138 g, 0.232 mmol, 89.7%) as a white foam. UPLC/ELSD: RT=2.56 min. MS (ES): m/z=557.3 [M+H]⁺ for C₃₆H₆₄N₂O₂; ¹H NMR (300 MHz, CDCl₃): δ 5.34-5.40 (m, 1H), 5.09 (m, 1H), 4.41-4.56 (m, 1H), 3.17 (dt, 2H, J=5.9, 5.8 Hz), 2.17-2.44 (m, 4H), 2.21 (s, 6H), 1.77-2.05 (br. m, 5H), 0.88-1.73 (br. m, 26H), 1.01 (s, 3H), 0.92 (d, 3H, J=6.5 Hz), 0.77-0.87 (m, 9H), 0.68 (s, 3H).

### E. Compound SA48: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(bis(2-hydroxyethyl)amino)ethyl)carbamate

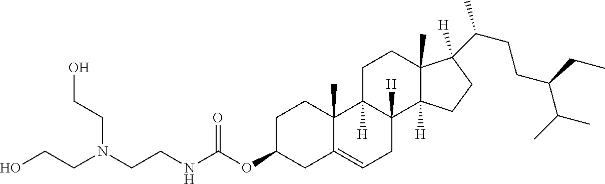

β-Sitosterol 4-nitrophenyl carbonate (0.150 g, 0.259 mmol), triethylamine (0.06 mL, 0.4 mmol), and 2-[(2-aminoethyl)(2-hydroxyethyl)amino]ethanol (0.05 mL, 0.4 mmol) were combined in CHCl₃ (2.5 mL) and stirred at 50° C. The reaction was monitored by TLC. At 21.5 hrs, the reaction mixture was cooled to rt and diluted with DCM (10 mL). The organics were washed with a 5% aq. NaHCO₃ solution. The aqueous layer was extracted with DCM, and then the combined organics were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(bis(2-hydroxyethyl)amino)ethyl)carbamate (0.056 g, 0.087 mmol, 33.6%) as a white solid. UPLC/ELSD: RT=2.43 min. MS (ES): m/z=589.2 [M+H]⁺ for C₃₆H₆₄N₂O₄; ¹H NMR (300 MHz, CDCl₃): δ 5.33-5.41 (m, 1H), 5.21 (br. s, 1H), 4.42-4.58 (m, 1H), 3.62 (t, 4H, J=5.0 Hz), 3.01-3.38 (br. m, 4H), 2.59-2.76 (m, 6H), 2.20-2.42 (m, 2H), 1.76-2.06 (br. m, 5H), 0.88-1.72 (br. m, 22H), 1.00 (s, 3H), 0.92 (d, 3H, J=6.4 Hz), 0.78-0.87 (m, 9H), 0.67 (s, 3H).

### F. Compound SA49: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(2-(dimethylamino)ethoxy)ethyl)carbamate

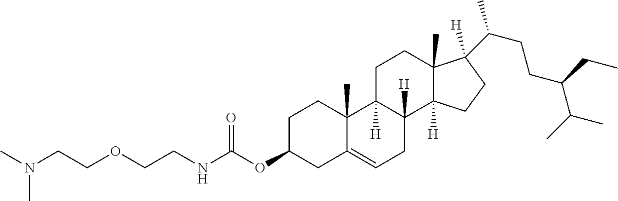

To a solution of β-sitosterol 4-nitrophenyl carbonate (0.150 g, 0.259 mmol), triethylamine (0.06 mL, 0.4 mmol) in CHCl₃ (2.5 mL) was added a solution of [2-(2-aminoethoxy)ethyl]dimethylamine (0.049 g, 0.37 mmol) in CHCl₃ (0.5 mL). The reaction mixture stirred at 50° C. and was monitored by TLC. At 21.5 hrs, the reaction mixture was cooled to rt and diluted with DCM (10 mL). The organics were washed with a 5% aq. NaHCO₃ solution. The aqueous was extracted with DCM, and then the combined organics were passed through a hydrophobic frit, dried over Na₂SO₄, and concentrated. The crude material was purified via silica gel chromatography (0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(2-(dimethylamino)ethoxy)ethyl)carbamate (0.123 g, 0.201 mmol, 77.8%) as a white foam. UPLC/ELSD: RT=2.55 min. MS (ES): m/z=573.3 [M+H]⁺ for C₃₆H₆₄N₂O₃; ¹H NMR (300 MHz, CDCl₃): δ 5.46-5.57 (m, 1H), 5.33-5.41 (m, 1H), 4.41-4.58 (m, 1H), 3.55 (t, 2H, J=5.6 Hz), 3.53 (t, 2H, J=4.5 Hz), 3.35 (dt, 2H, J=5.1, 5.0 Hz), 2.49 (t, 2H, J=5.6 Hz), 2.21-2.42 (m, 2H), 2.27 (s, 6H), 1.76-2.07 (br. m, 5H), 0.88-1.74 (br. m, 22H), 1.01 (s, 3H), 0.92 (d, 3H, J=6.4 Hz), 0.78-0.87 (m, 9H), 0.68 (s, 3H).

### G. Compound SA52: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-(4,7-dimethyl-1,4,7-triazonan-1-yl)-4-oxobutanoate

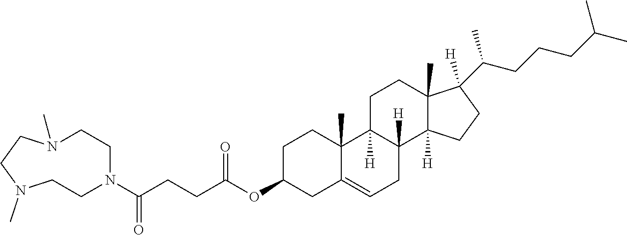

To a stirred solution of cholesteryl hemisuccinate (0.120 g, 0.247 mmol), 1,4-dimethyl-1,4,7-triazonane (Enamine, Monmouth Junction, NJ) (0.042 g, 0.27 mmol), and DMAP (cat.) in DCM (1.5 mL) was added 1-ethyl-3-(3-dimethylaminopropyl)carbodiimide hydrochloride (0.071 g, 0.37 mmol). The reaction mixture stirred at rt and was monitored by LCMS. At 46 h, water (2 mL) was added. The mixture stirred at rt for 16 h, then was diluted with 5% aq. NaHCO₃ solution (5 mL) and then extracted with DCM (3×10 mL). The combined organics were passed through a hydrophobic frit, dried over Na₂SO₄, and concentrated. The crude material was purified via silica gel chromatography (0-12% (5% conc. aq. NH₄OH in MeOH) in DCM). The material was purified again via silica gel chromatography (0-10% (5% conc. aq. NH₄OH in MeOH) in DCM) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(4,7-dimethyl-1,4,7-triazonan-1-yl)-4-oxobutanoate (0.100 g, 0.140 mmol, 56.7%) as a clear oil. UPLC/ELSD: RT=2.52 min. MS (ES): m/z=626.2 [M+H]⁺ for C₃₉H₆₇N₃O₃; ¹H NMR (300 MHz, CDCl₃): δ 5.31-5.40 (m, 1H), 4.52-4.69 (m, 1H), 3.40-3.58 (m, 4H), 2.89-2.98 (m, 2H), 2.71-2.81 (m, 2H), 2.61-2.68 (m, 4H), 2.45-2.53 (m, 4H), 2.27-2.42 (m, 2H), 2.40 (s, 3H), 2.35 (s, 3H), 1.74-2.04 (br. m, 5H), 0.93-1.68 (m, 21H), 1.00 (s, 3H), 0.90 (d, 3H, J=6.4 Hz), 0.86 (d, 3H, J=6.6 Hz), 0.85 (d, 3H, J=6.6 Hz), 0.67 (s, 3H).

### H. Compound SA53: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)-2-methylpropyl)carbamate

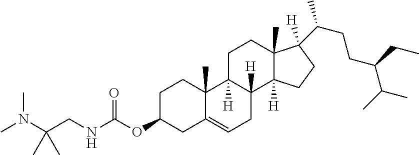

β-Sitosterol 4-nitrophenyl carbonate (0.150 g, 0.259 mmol), (1-amino-2-methylpropan-2-yl)dimethylamine (0.036 g, 0.31 mmol), and triethylamine (0.06 mL, 0.4 mmol) were combined in CHCl₃ (2.4 mL) and stirred at 50° C. The reaction was monitored by TLC. At 28 h, triethylamine (0.03 mL) and (1-amino-2-methylpropan-2-yl)dimethylamine (22 mg) were added. The reaction mixture stirred at 55° C. At 46 h, the reaction mixture was cooled to rt, diluted with a 5% aq. NaHCO₃ solution (10 mL), and extracted with DCM (2×10 mL). The combined organics were passed through a hydrophobic frit, dried over Na₂SO₄, and concentrated. The crude material was purified via silica gel chromatography (0-15% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-(dimethylamino)-2-methylpropyl)carbamate (0.091 g, 0.16 mmol, 60.4%) as an off-white solid. UPLC/ELSD: RT=2.55 min. MS (ES): m/z=557.4 [M+H]⁺ for C₃₆H₆₄N₂O₂; ¹H NMR (300 MHz, CDCl₃): δ 5.34-5.43 (m, 1H), 5.12-5.31 (m, 1H), 4.42-4.59 (m, 1H), 3.09 (m, 2H), 2.20-2.43 (m, 2H), 2.17 (s, 6H), 1.75-2.06 (br. m, 5H), 0.88-1.74 (br. m, 22H), 1.01 (s, 3H), 0.99 (s, 6H), 0.92 (d, 3H, J=6.5 Hz), 0.77-0.88 (m, 9H), 0.68 (s, 3H).

### I. Compound SA54: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl ((1-(dimethylamino)cyclopropyl)methyl)carbamate

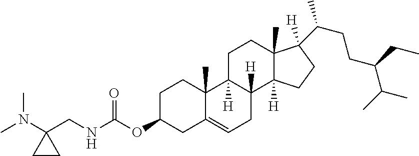

β-Sitosterol 4-nitrophenyl carbonate (0.150 g, 0.259 mmol), 1-(aminomethyl)-N,N-dimethylcyclopropan-1-amine (0.035 g, 0.31 mmol) and triethylamine (0.06 mL, 0.4 mmol) were combined in CHCl₃ (2.4 mL) and stirred at 50° C. The reaction was monitored by TLC. At 28 hrs, triethylamine (0.03 mL) and 1-(aminomethyl)-N,N-dimethylcyclopropan-1-amine (22 mg) were added. The reaction mixture stirred at 55° C. At 46 hrs, the reaction mixture was cooled to rt, diluted with 5% aq. NaHCO₃ solution (10 mL), and then extracted with DCM (2×10 mL). The combined organics were passed through a hydrophobic frit, dried over Na₂SO₄, and concentrated. The crude material was purified via silica gel chromatography (0-15% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl ((1-(dimethylamino)cyclopropyl)methyl)carbamate (0.130 g, 0.225 mmol, 86.9%) as an off-white solid. UPLC/ELSD: RT=2.57 min. MS (ES): m/z=555.3 [M+H]⁺ for C₃₆H₆₂N₂O₂; ¹H NMR (300 MHz, CDCl₃): δ 5.34-5.41 (m, 1H), 4.68 (br. s, 1H), 4.41-4.57 (m, 1H), 3.28 (d, 2H, J=4.9 Hz), 2.16-2.57 (m, 2H), 2.40 (s, 6H), 1.76-2.08 (br. m, 5H), 0.88-1.74 (br. m, 22H), 1.01 (s, 3H), 0.92 (d, 3H, J=6.5 Hz), 0.77-0.87 (m, 9H), 0.68 (s, 3H), 0.65 (br. s, 2H), 0.53 (br. s, 2H).

### J. Compound SA55: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-amino-2-methylpropyl)carbamate hydrochloride

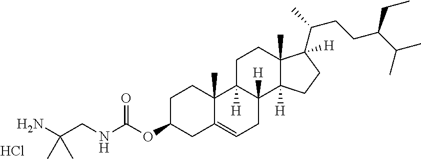

### Step 1: tert-Butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) (2,2-dimethylethane-1,2-diyl)dicarbamate

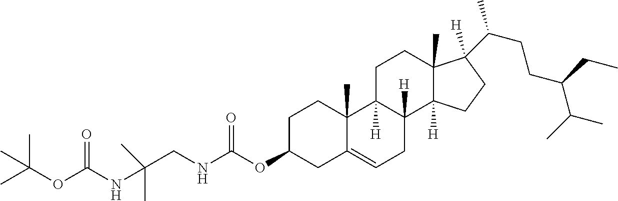

β-Sitosterol 4-nitrophenyl carbonate (0.225 g, 0.388 mmol), tert-butyl N-(1-amino-2-methylpropan-2-yl)carbamate (0.088 g, 0.47 mmol) and triethylamine (0.08 mL, 0.6 mmol) were combined in CHCl₃ (3.6 mL) and stirred at 50° C. The reaction was monitored by TLC. At 28 h, triethylamine (0.04 mL) and tert-butyl N-(1-amino-2-methylpropan-2-yl)carbamate (41 mg) were added. The reaction mixture stirred at 55° C. At 46 hrs, the reaction mixture was cooled to rt, diluted with 5% aq. NaHCO₃ solution (10 mL), and then extracted with DCM (2×10 mL). The combined organics were passed through a hydrophobic frit, dried over Na₂SO₄, and concentrated. The crude material was purified via silica gel chromatography (0-20% (5% conc. aq. NH₄OH in MeOH) in DCM) to afford tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) (2,2-dimethylethane-1,2-diyl)dicarbamate (0.220 g, 0.350 mmol, 90.1%) as a white solid. ¹H NMR (300 MHz, CDCl₃): δ 5.34-5.43 (m, 1H), 5.16 (br. s, 1H), 4.41-4.69 (m, 2H), 3.35 (d, 2H, J=6.3 Hz), 2.21-2.43 (m, 2H), 1.76-2.08 (br. m, 5H), 0.88-1.73 (br. m, 22H), 1.43 (s, 9H), 1.25 (s, 6H), 1.01 (s, 3H), 0.92 (d, 3H, J=6.4 Hz), 0.78-0.88 (m, 9H), 0.68 (s, 3H). UPLC/ELSD: RT=3.40 min. MS (ES): m/z=651.1 [M+Na]⁺ for C₃₉H₆₈N₂O₄.

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-JH-cyclopenta[a]phenanthren-3-yl (2-amino-2-methylpropyl)carbamate hydrochloride

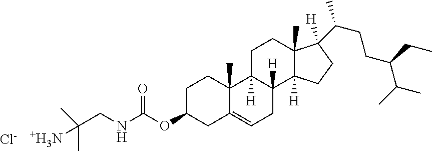

To a solution of tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) (2,2-dimethylethane-1,2-diyl)dicarbamate (0.211 g, 0.335 mmol) in iPrOH (2.1 mL) was added 5-6 N HCl in iPrOH (0.35 mL). The reaction mixture stirred at 40° C. and was monitored by LCMS. At 17 h, the reaction mixture was cooled to rt. ACN (4 mL) was added, and the suspension was cooled to 0° C. in an ice bath. Solids were collected by vacuum filtration and rinsed with 2:1 ACN:iPrOH to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (2-amino-2-methylpropyl)carbamate hydrochloride (0.174 g, 0.292 mmol, 87.0%) as a white solid. UPLC/ELSD: RT=2.51 min. MS (ES): m/z=529.3 [M+H]⁺ for C₃₄H₆₀N₂O₂; ¹H NMR (300 MHz, CD₃OD): δ 5.36-5.47 (m, 1H), 4.37-4.53 (m, 1H), 3.24 (s, 2H), 2.28-2.46 (m, 2H), 1.81-2.13 (br. m, 5H), 0.92-1.77 (br. m, 22H), 1.31 (s, 6H), 1.06 (s, 3H), 0.96 (d, 3H, J=6.4 Hz), 0.81-0.91 (m, 9H), 0.74 (s, 3H).

### K. Compound SA85: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)carbamate hydrochloride

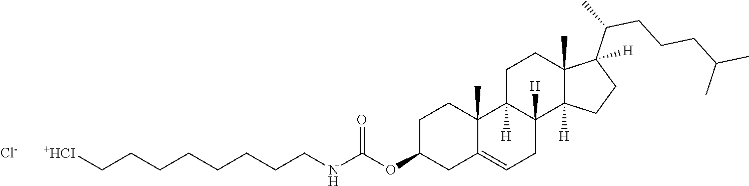

### Step 1: Tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) octane-1,8-diyldicarbamate

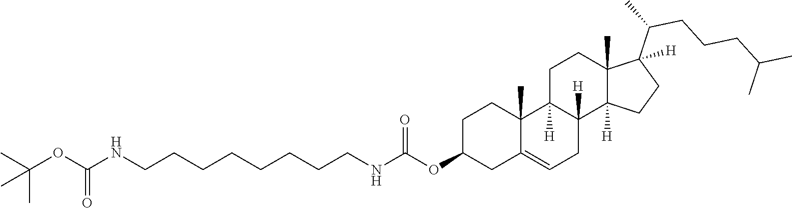

Cholesterol 4-nitrophenyl carbonate (0.200 g, 0.362 mmol), tert-butyl N-(8-aminooctyl)carbamate (0.106 g, 0.435 mmol), triethylamine (0.15 mL, 1.1 mmol) were combined in toluene (3.5 mL). The reaction mixture stirred at 90° C. and was monitored by LCMS. At 18 h, the reaction mixture was cooled to rt, diluted with dichloromethane (25 mL), then washed with 5% aq. NaHCO₃ solution (3×25 mL). The organics were passed through a hydrophobic frit, dried over Na₂SO₄, and concentrated. The crude material was purified via silica gel chromatography (0-30% ethyl acetate in hexanes) to afford tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) octane-1,8-diyldicarbamate (0.236 g, 0.359 mmol, 99.1%) as a clear oil. UPLC/ELSD: RT=3.34 min. MS (ES): m/z=658.36 [M+H]⁺ for C₄₁H₇₂N₂O₄; ¹H NMR (300 MHz, CDCl₃): δ 5.33-5.42 (m, 1H), 4.38-4.66 (m, 3H), 3.03-3.24 (m, 4H), 2.19-2.43 (m, 2H), 1.75-2.17 (m, 5H), 0.94-1.67 (br. m, 33H), 1.44 (s, 9H), 1.01 (s, 3H), 0.91 (d, 3H, J=6.4 Hz), 0.87 (d, 3H, J=6.6 Hz), 0.86 (d, 3H, J=6.5 Hz), 0.68 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)carbamate hydrochloride

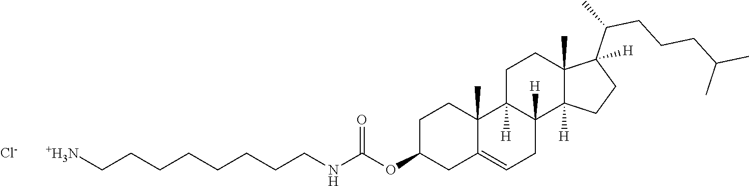

To a solution of tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) octane-1,8-diyldicarbamate (0.236 g, 0.359 mmol) in isopropanol (3.5 mL) was added 5-6 N HCl in isopropanol (0.25 mL). The reaction mixture stirred at 40° C. and was monitored by LCMS. At 16 h, 5-6 N HCl in isopropanol (0.25 mL) was added. At 20 h, acetonitrile (10.5 mL) was added, and the suspension was stirred at rt for 5 min. Then solids were collected by vacuum filtration rinsing with 3:1 acetonitrile/isopropanol to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)carbamate hydrochloride (0.126 g, 0.208 mmol, 57.9%) as a white solid. UPLC/ELSD: RT=2.62 min. MS (ES): m/z=558.16 [M+H]⁺ for C₃₆H₆₄N₂O₂; ¹H NMR (300 MHz, CD₃OD): δ 5.34-5.44 (m, 1H), 4.28-4.46 (m, 1H), 3.07 (t, 2H, J=6.4 Hz), 2.91 (t, 2H, J=7.0 Hz), 2.22-2.40 (m, 2H), 1.79-2.12 (m, 5H), 0.80-1.74 (br. m, 45H), 0.72 (s, 3H).

### L. Compound SA86: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)carbamate hydrochloride

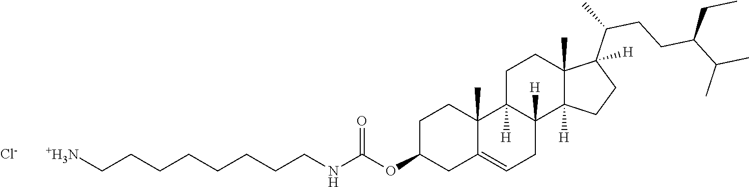

### Step 1: Tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) octane-1,8-diyldicarbamate

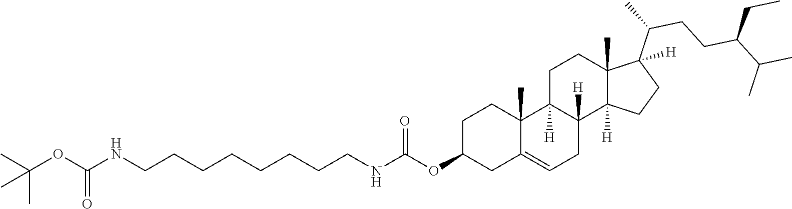

β-Sitosterol 4-nitrophenyl carbonate (0.200 g, 0.345 mmol), tert-butyl N-(8-aminooctyl)carbamate (0.105 g, 0.431 mmol), and triethylamine (0.15 mL, 1.1 mmol) were combined in toluene (3.5 mL). The reaction mixture stirred at 90° C. and was monitored by LCMS. At 18 h, the reaction mixture was cooled to rt, diluted with dichloromethane (25 mL) and then washed with 5% aq. NaHCO₃ solution (3×25 mL). The combined organics were passed through a hydrophobic frit, dried over Na₂SO₄, and concentrated. The crude material was purified via silica gel chromatography (0-30% ethyl acetate in hexanes) to afford tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) octane-1,8-diyldicarbamate (0.199 g, 0.29 mmol, 84.2%) as a white foam. UPLC/ELSD: RT=3.44 min. MS (ES): m/z=629.86 [(M+H)—(CH₃)₂C═CH₂]⁺ for C₄₃H₇₆N₂O₄; ¹H NMR (300 MHz, CDCl₃): δ 5.33-5.45 (m, 1H), 4.30-4.71 (m, 3H), 2.99-3.28 (m, 4H), 2.18-2.45 (m, 2H), 1.76-2.11 (m, 5H), 0.88-1.73 (br. m, 34H), 1.44 (s, 9H), 1.01 (s, 3H), 0.92 (d, 3H, J=6.4 Hz), 0.77-0.88 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)carbamate hydrochloride

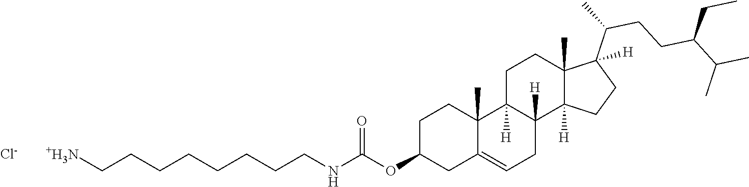

To a solution of tert-butyl ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl) octane-1,8-diyldicarbamate (0.188 g, 0.274 mmol) in isopropanol (2.8 mL) was added 5-6 N HCl in isopropanol (0.20 mL). The reaction mixture stirred at 40° C. and was monitored by LCMS. At 16 h, 5-6 N HCl in isopropanol (0.20 mL) was added. At 20 h, acetonitrile (8.4 mL) was added, and the suspension was stirred at rt for 5 min. Then solids were collected by vacuum filtration rinsing with 3:1 acetonitrile/isopropanol to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)carbamate hydrochloride (0.107 g, 0.162 mmol, 59.2%) as a white solid. UPLC/ELSD: RT=2.73 min. MS (ES): m/z=585.68 [M+H]⁺ for C₃₈H₆₈N₂O₂; ¹H NMR (300 MHz, CD₃OD): δ 5.33-5.45 (m, 1H), 4.27-4.47 (m, 1H), 3.07 (t, 2H, J=6.3 Hz), 2.91 (t, 2H, J=6.6 Hz), 2.21-2.40 (m, 2H), 1.79-2.12 (m, 5H), 0.79-1.77 (br. m, 49H), 0.73 (s, 3H).

### M. Compound SA91: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((2-aminoethyl)amino)-2-oxoethyl)disulfaneyl)acetate hydrochloride

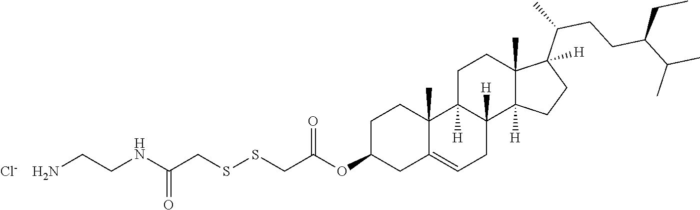

### Step 1: 2-((2-(((3S,8S,9S,10R,13R,14S,17R)-10,13-dimethyl-17-((R)-6-methylheptan-2-yl)-2,3,4, 7, 8,9,10,11,12,13,14,15,16,17-tetradecahydro-1H-cyclopenta[a]phenanthren-3-yl)oxy)-2-oxoethyl)disulfaneyl)acetic acid

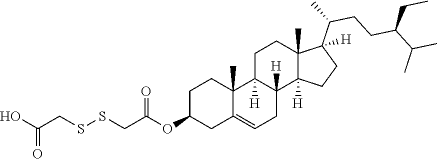

To a solution of cholesterol (5.00 g, 12.93 mmol) in dry DCM (100 mL) stirring under nitrogen was added dithiodiglycolic acid (4.53 mL, 25.86 mmol). The solution was then cooled to 0° C., and dimethylaminopyridine (0.32 g, 2.59 mmol) and 1-ethyl-3-(3-dimethylaminopropyl)carbodiimide hydrochloride (4.96 g, 25.86 mmol) were added, followed by dropwise addition of triethylamine (4.52 mL, 25.86 mmol). The solution was allowed to gradually warm to room temperature and stir overnight. The following day, the solution was washed with saturated sodium bicarbonate (1×25 mL) and water (1×25 mL), dried over sodium sulfate, filtered, and concentrated to a brown oil. The oil was taken up in DCM and purified on silica in hexanes with a 0-100% EtOAc gradient. Product-containing fractions were pooled and concentrated to give 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid as a dark brown solid (3.76 g, 6.82 mmol, 52.7%). UPLC/ELSD: RT: 3.11 min. MS (ES): m/z (MH⁺) 551.8 for C₃₁H₅₀O₄S₂. ¹H NMR (300 MHz, CDCl₃) δ: ppm 9.04 (br. s, 1H), 5.41 (m, 1H), 4.69 (br. m, 1H), 3.65 (s, 2H), 3.60 (s, 1H), 2.39 (d, 2H, J=9 Hz), 2.01 (br. m, 5H), 1.52 (br. m, 11H), 1.16 (br. m, 6H), 1.04 (s, 6H), 0.95 (d, 3H, J=6 Hz), 0.86 (d, 6H, J=6 Hz), 0.70 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 13,13-dimethyl-6,11-dioxo-12-oxa-3,4-dithia-7,10-diazatetradecanoate

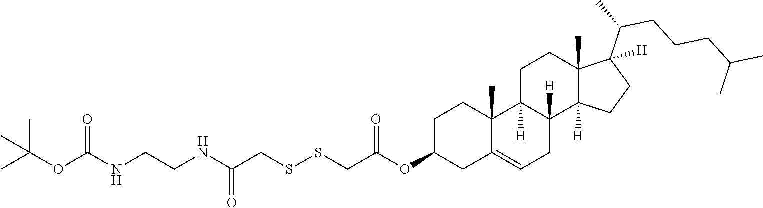

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.31 g, 0.57 mmol) in dry DCM (5 mL) stirring under nitrogen was added tert-butyl (2-aminoethyl)carbamate (0.14 mL, 0.85 mmol), dimethylaminopyridine (0.01 g, 0.06 mmol), 1-ethyl-3-(3-dimethylaminopropyl)carbodiimide hydrochloride (0.16 g, 0.85 mmol), and diisopropylethylamine (0.30 mL, 1.71 mmol). The solution was allowed to stir overnight at room temperature. The following day, the solution was washed with saturated sodium bicarbonate (1×5 mL) and brine (1×5 mL), dried over sodium sulfate, filtered, and concentrated to a white solid. The solid was taken up in DCM and purified on silica in DCM with a 0-60% (75:20:5 DCM/MeOH/aqueous NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 13,13-dimethyl-6,11-dioxo-12-oxa-3,4-dithia-7,10-diazatetradecanoate as a yellow oil (0.06 g, 0.08 mmol, 14.0%). UPLC/ELSD: RT: 3.19 min. MS (ES): m/z (MH⁺) 694.1 for C₃₈H₆₄N₂O₅S₂. ¹H NMR (300 MHz, CDCl₃) δ: ppm 5.40 (m, 1H), 4.72 (br. m, 3H), 4.12 (m, 1H), 3.79 (m, 2H), 3.56 (s, 2H), 3.48 (m, 6H), 3.33 (br. m, 3H), 2.38 (d, 2H, J=9 Hz), 1.88 (br. m, 11H), 1.46 (s, 24H), 1.27 (br. m, 12H), 1.04 (s, 6H), 0.94 (d, 4H, J=6 Hz), 0.89 (d, 6H, J=6 Hz), 0.69 (s, 3H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((2-aminoethyl)amino)-2-oxoethyl)disulfaneyl)acetate hydrochloride

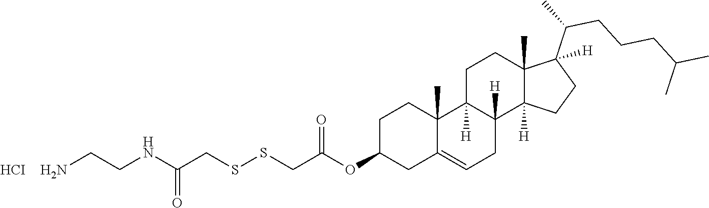

To a solution (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 13,13-dimethyl-6,11-dioxo-12-oxa-3,4-dithia-7,10-diazatetradecanoate (0.09 g, 0.12 mmol) in isopropanol (5 mL) set stirring under nitrogen was added hydrochloric acid (5 N in isopropanol, 0.25 mL, 1.23 mmol) dropwise. The solution was heated to 40° C. and allowed to proceed overnight. The following morning, the solution was cooled to room temperature and dry acetonitrile (10 mL) was added to the mixture. The mixture was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((2-aminoethyl)amino)-2-oxoethyl)disulfaneyl)acetate hydrochloride as a white solid (0.02 g, 0.03 mmol, 26.0%). UPLC/ELSD: RT=2.54 min. MS (ES): m/z (MH⁺) 593.7 for C₃₃H₅₇C₁N₂O₃S₂. ¹H NMR (300 MHz, MeOD) δ: ppm 8.41 (br. s, 1H), 5.43 (m, 1H), 4.62 (br. m, 2H), 4.03 (m, 1H), 3.65 (s, 3H), 3.57 (s, 3H), 3.11 (m, 3H), 2.37 (br. m, 2H), 1.93 (br. m, 5H), 1.55 (br. m, 11H), 1.17 (m, 6H), 1.08 (s, 4H), 0.98 (d, 3H, J=6 Hz), 0.91 (d, 5H, J=6 Hz), 0.75 (s, 3H).

### N. Compound SA92: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((6-aminohexyl)amino)-2-oxoethyl)disulfaneyl)acetate hydrochloride

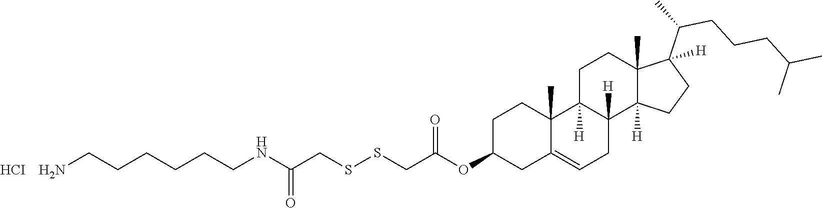

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 17,17-dimethyl-6,15-dioxo-16-oxa-3,4-dithia-7,14-diazaoctadecanoate

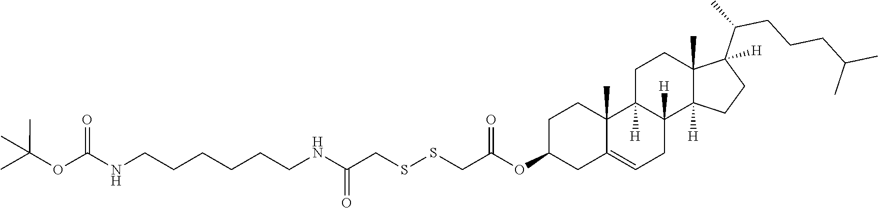

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.30 g, 0.55 mmol) in dry DCM (5 mL) stirring under nitrogen was added tert-butyl (6-aminohexyl)carbamate (0.25 mL, 1.09 mmol), dimethylaminopyridine (0.03 g, 0.27 mmol), and 1-ethyl-3-(3-dimethylaminopropyl)carbodiimide hydrochloride (0.21 g, 1.09 mmol). The solution was allowed to stir overnight at room temperature. The following day, the solution was diluted with DCM, washed with saturated sodium bicarbonate (1×10 mL) and brine (1×10 mL), dried over sodium sulfate, filtered, and concentrated to a white solid. The solid was taken up in DCM and purified on silica in hexanes with a 0-80% EtOAc gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 17,17-dimethyl-6,15-dioxo-16-oxa-3,4-dithia-7,14-diazaoctadecanoate as a light yellow oil (0.11 g, 0.14 mmol, 26.2%). UPLC/ELSD: RT: 3.28 min. MS (ES): m/z (MH⁺) 750.1 for C₄₂H₇₂N₂O₅S₂. ¹H NMR (300 MHz, CDCl₃) δ: ppm 6.80 (br. s, 1H), 5.40 (br. m, 1H), 4.66 (br. m, 2H), 3.66 (m, 1H), 3.54 (s, 2H), 3.46 (s, 2H), 3.31 (br. m, 3H), 3.11 (br. m, 3H), 2.37 (d, 2H, J=9 Hz), 2.04 (br. m, 6H), 1.56 (br. m, 7H), 1.44 (s, 21H), 1.35 (br. m, 10H), 1.14 (m, 7H), 1.03 (s, 6H), 0.90 (d, 4H, J=6 Hz), 0.87 (d, 6H, J=6 Hz), 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((6-aminohexyl)amino)-2-oxoethyl)disulfaneyl)acetate hydrochloride

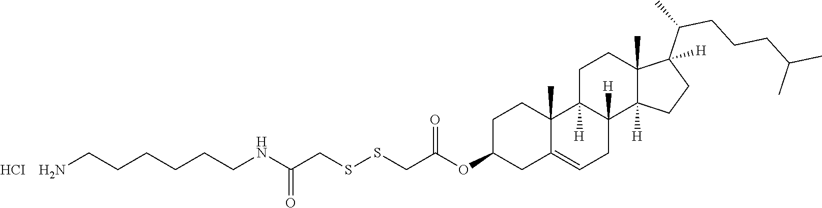

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 17,17-dimethyl-6,15-dioxo-16-oxa-3,4-dithia-7,14-diazaoctadecanoate (0.11 g, 0.14 mmol) in isopropanol (5 mL) set stirring under nitrogen was added hydrochloric acid (5 N in isopropanol, 0.29 mL, 1.43 mmol) dropwise.

The solution was heated to 40° C. and allowed to proceed overnight. The following morning, the solution was cooled to room temperature and dry acetonitrile (10 mL) was added to the mixture. The mixture was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((6-aminohexyl)amino)-2-oxoethyl)disulfaneyl)acetate hydrochloride as a white solid (0.04 g, 0.05 mmol, 34.4%). UPLC/ELSD: RT=2.53 min. MS (ES): m/z (MH⁺) 650.0 for C₃₇H₆₅ClN₂O₃S₂. ¹H NMR (300 MHz, MeOD) δ: ppm 5.32 (m, 1H), 4.49 (br. m, 1H), 3.83 (m, 1H), 3.58 (m, 2H), 3.52 (s, 2H), 3.38 (s, 2H), 3.22 (m, 5H), 3.14 (br. m, 3H), 2.83 (t, 5H), 2.25 (m, 2H), 1.86 (br. m, 7H), 1.58 (m, 19H), 1.33 (br. m, 17H), 1.06 (d, 13H, J=6 Hz), 0.96 (s, 7H), 0.86 (d, 5H, J=6 Hz), 0.80 (d, 8H, J=6 Hz), 0.63 (s, 4H).

### O. Compound SA93: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((8-aminooctyl)amino)-2-oxoethyl)disulfaneyl)acetate hydrochloride

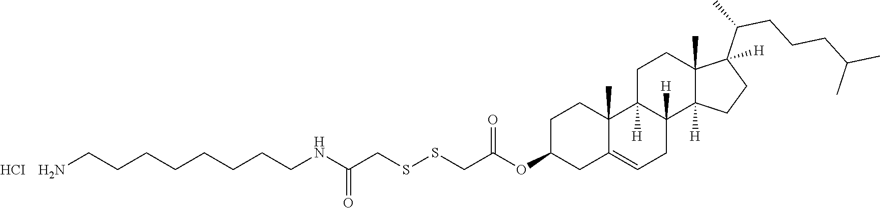

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 19,19-dimethyl-6,17-dioxo-18-oxa-3,4-dithia-7,16-diazaicosanoate

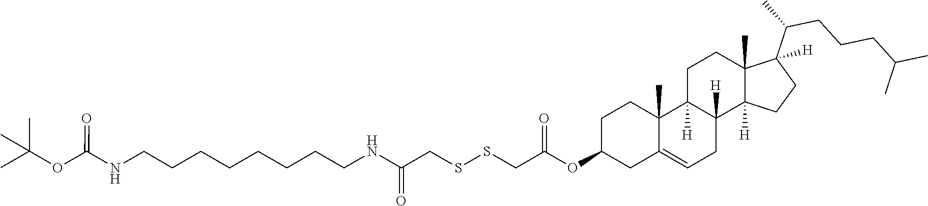

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.30 g, 0.55 mmol) in dry DCM (5 mL) stirring under nitrogen was added tert-butyl (8-aminooctyl)carbamate (0.27 mL, 1.09 mmol), dimethylaminopyridine (0.03 g, 0.27 mmol), and 1-ethyl-3-(3-dimethylaminopropyl)carbodiimide hydrochloride (0.21 g, 1.09 mmol). The solution was allowed to stir overnight at room temperature. The following day, the solution was diluted with DCM, washed with saturated sodium bicarbonate (1×10 mL) and brine (1×10 mL), dried over sodium sulfate, filtered, and concentrated to a white solid. The solid was taken up in DCM and purified on silica in hexanes with a 0-80% EtOAc gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 19,19-dimethyl-6,17-dioxo-18-oxa-3,4-dithia-7,16-diazaicosanoate as a light yellow oil (0.11 g, 0.14 mmol, 26.9%). UPLC/ELSD: RT: 3.34 min. MS (ES): m/z (MH⁺) 778.1 for C₄₄H₇₆N₂O₅S₂. ¹H NMR (300 MHz, CDCl₃) δ: ppm 6.75 (br. s, 1H), 5.40 (br. m, 1H), 4.66 (br. m, 2H), 3.54 (s, 2H), 3.46 (s, 2H), 3.28 (br. m, 2H), 3.08 (br. m, 2H), 2.37 (d, 2H, J=9 Hz), 1.91 (br. m, 6H), 1.44 (br. s, 22H), 1.31 (br. m, 13H), 1.11 (m, 7H), 1.03 (s, 6H), 0.93 (d, 4H, J=6 Hz), 0.88 (d, 6H, J=6 Hz), 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((8-aminooctyl)amino)-2-oxoethyl)disulfaneyl)acetate hydrochloride

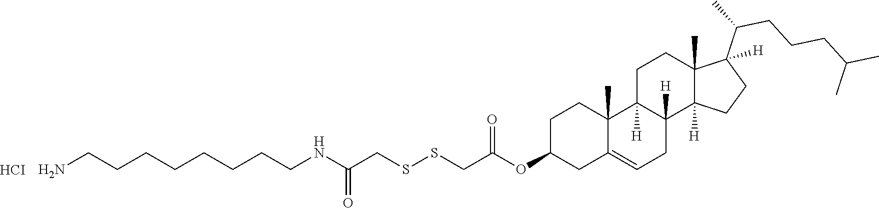

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 19,19-dimethyl-6,17-dioxo-18-oxa-3,4-dithia-7,16-diazaicosanoate (0.11 g, 0.15 mmol) in isopropanol (5 mL) set stirring under nitrogen was added hydrochloric acid (5 N in isopropanol, 0.29 mL, 1.47 mmol) dropwise. The solution was heated to 40° C. and allowed to proceed overnight. The following morning, the solution was cooled to room temperature and dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((8-aminooctyl)amino)-2-oxoethyl)disulfaneyl)acetate hydrochloride as a white solid (0.03 g, 0.04 mmol, 26.7%). UPLC/ELSD: RT=2.52 min. MS (ES): m/z (MH⁺) 677.9 for C₃₉H₆₉C₁N₂O₃S₂. ¹H NMR (300 MHz, MeOD) δ: ppm 5.31 (m, 1H), 4.48 (br. m, 1H), 3.82 (m, 1H), 3.51 (s, 2H), 3.36 (s, 2H), 3.21 (m, 7H), 3.12 (br. m, 2H), 2.81 (t, 2H), 2.27 (m, 2H), 1.94 (br. m, 11H), 1.53 (m, 18H), 1.28 (br. m, 15H), 1.04 (d, 14H, J=6 Hz), 0.96 (m, 10H), 0.86 (d, 6H, J=6 Hz), 0.80 (d, 10H, J=6 Hz), 0.63 (s, 5H).

### P. Compound SA94: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((10-aminodecyl)amino)-2-oxoethyl)disulfaneyl)acetate hydrochloride

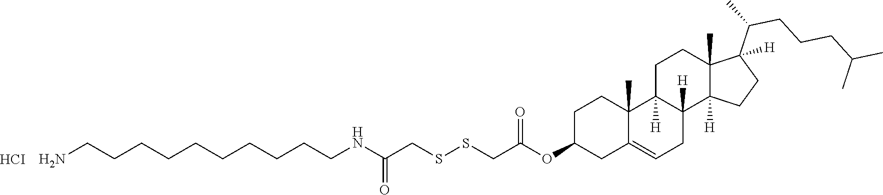

### Step 1: (3S,8S,9S,10R,13R,14S,17R)-10,13-dimethyl-17-((R)-6-methylheptan-2-yl)-2,3,4, 7, 8,9,10,11,12,13,14,15,16,17-tetradecahydro-1H-cyclopenta[a]phenanthren-3-yl 21,21-dimethyl-6,19-dioxo-20-oxa-3,4-dithia-7,18-diazadocosanoate

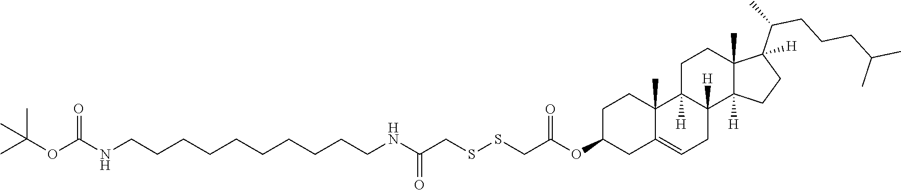

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.30 g, 0.55 mmol) in dry DCM (5 mL) stirring under nitrogen was added tert-butyl (10-aminodecyl)carbamate (0.31 g, 1.09 mmol), dimethylaminopyridine (0.03 g, 0.27 mmol), and 1-ethyl-3-(3-dimethylaminopropyl)carbodiimide hydrochloride (0.21 g, 1.09 mmol). The solution was allowed to stir overnight at room temperature. The following day, the solution was diluted with DCM, washed with saturated sodium bicarbonate (1×10 mL) and brine (1×10 mL), dried over sodium sulfate, filtered, and concentrated to a white solid. The solid was taken up in DCM and purified on silica in hexanes with a 0-80% EtOAc gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 21,21-dimethyl-6,19-dioxo-20-oxa-3,4-dithia-7,18-diazadocosanoate as a light yellow oil (0.14 g, 0.17 mmol, 31.5%). UPLC/ELSD: RT: 3.43 min. MS (ES): m/z (MH⁺) 806.3 for C₄₆H₈₀N₂O₅S₂. ¹H NMR (300 MHz, CDCl₃) δ: ppm 6.75 (br. s, 1H), 5.40 (br. m, 1H), 4.67 (br. m, 2H), 3.53 (s, 2H), 3.46 (s, 2H), 3.28 (br. m, 2H), 3.10 (br. m, 2H), 2.34 (d, 2H, J=9 Hz), 2.00 (br. m, 5H), 1.44 (br. s, 20H), 1.27 (br. m, 16H), 1.11 (m, 7H), 1.03 (s, 6H), 0.90 (d, 4H, J=6 Hz), 0.87 (d, 6H, J=6 Hz), 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((10-aminodecyl)amino)-2-oxoethyl)disulfaneyl)acetate hydrochloride

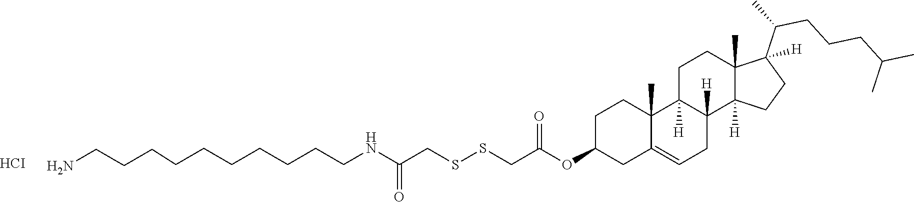

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 21,21-dimethyl-6,19-dioxo-20-oxa-3,4-dithia-7,18-diazadocosanoate (0.14 g, 0.17 mmol) in isopropanol (5 mL) set stirring under nitrogen was added hydrochloric acid (5 N in isopropanol, 0.34 mL, 1.71 mmol) dropwise. The solution was heated to 40° C. and allowed to proceed overnight. The following morning, the solution was cooled to room temperature, and dry acetonitrile (10 mL) was added to the mixture. The mixture was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((10-aminodecyl)amino)-2-oxoethyl)disulfaneyl)acetate hydrochloride as a white solid (0.05 g, 0.07 mmol, 38.1%). UPLC/ELSD: RT=2.62 min. MS (ES): m/z (MH⁺) 705.9 for C₄₁H₇₃ClN₂O₃S₂. ¹H NMR (300 MHz, MeOD) δ: ppm 5.32 (m, 1H), 4.49 (br. m, 1H), 3.82 (m, 2H), 3.51 (s, 2H), 3.37 (s, 2H), 3.21 (m, 3H), 3.11 (t, 2H), 2.84 (t, 2H), 2.25 (m, 2H), 2.14 (br. m, 1H), 1.94 (br. m, 8H), 1.53 (m, 15H), 1.25 (br. m, 18H), 1.06 (d, 21H, J=6 Hz), 0.96 (m, 8H), 0.86 (d, 6H, J=6 Hz), 0.80 (d, 9H, J=6 Hz), 0.63 (s, 5H).

### Q. Compound SA50: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate dihydrochloride

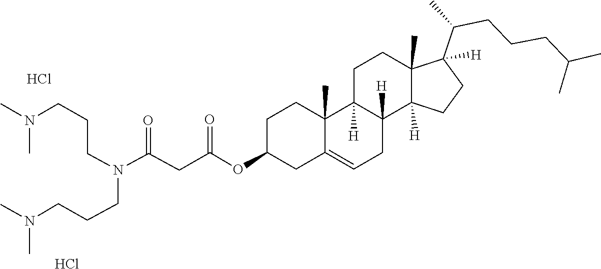

### Step 1: tert-Butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-H-cyclopenta[a]phenanthren-3-yl) malonate

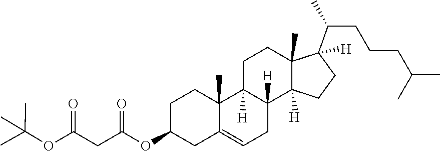

To a solution of cholesterol (4.00 g, 10.14 mmol) and 3-(tert-butoxy)-3-oxopropanoic acid (2.39 mL, 15.21 mmol) in dichloromethane (20 mL) stirring under nitrogen was added 1-ethyl-3-(3-dimethylaminopropyl)carbodiimide hydrochloride (2.95 g, 15.21 mmol). Then the reaction mixture was cooled to 0° C., and diisoproylethylamine (5.36 mL, 30.41 mmol) was added dropwise over 20 minutes. The resulting mixture was allowed to gradually warm to room temperature and proceed overnight. The mixture was then diluted with dichloromethane to 150 mL, washed with water (1×70 mL), saturated aqueous sodium bicarbonate (2×70 mL), and brine (1×70 mL), dried over sodium sulfate, filtered, and concentrated in vacuo to give a yellow oil. The oil was taken up in dichloromethane and purified on silica with a 0-25% ethyl acetate gradient in hexanes to give tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) malonate (4.99 g, 9.44 mmol, 93.1%) as an oil. UPLC/ELSD: RT: 3.36 min. MS (ES): m/z (MH⁺) 529.8 for C₃₄H₅₆O₄. ¹H NMR (300 MHz, CDCl₃) δ: ppm 5.41 (m, 1H), 4.67 (m, 1H), 3.27 (s, 2H), 2.38 (d, 2H), 1.91 (br. m, 10H), 1.49 (s, 12H), 1.35 (br. m, 6H), 1.04 (br. m, 17H), 0.91 (d, 3H, J=3 Hz), 0.87 (d, 3H, J=3 Hz), 0.70 (s, 3H).

### Step 2: 3-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-3-oxopropanoic acid

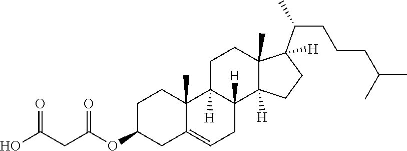

To a solution of tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) malonate (4.99 g, 9.44 mmol) in dichloromethane (50 mL) stirring under nitrogen at 0° C., was added trifluoroacetic acid (10.85 mL, 141.63 mmol) dropwise over 20 minutes. The clear, light yellow reaction mixture was allowed to gradually warm to room temperature and proceed overnight. The following morning, the reaction was quenched with 20 mL of a 5% aqueous sodium bicarbonate solution at 0° C. The organics were separated, washed with an additional 10 mL of 5% aqueous sodium bicarbonate, dried over sodium sulfate, filtered, and concentrated to give a white solid. The solid was taken up in dichloromethane and purified on silica with a 0-60% ethyl acetate gradient in hexanes to give 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-3-oxopropanoic acid (3.12 g, 6.61 mmol, 70.0%) as a white solid. UPLC/ELSD: RT: 2.97 min. MS (ES): m/z (MH⁺) 473.7 for C₃₀H₄₈O₄. ¹H NMR (300 MHz, CDCl₃) δ: ppm 10.99 (br. s, 1H), 5.42 (m, 1H), 4.73 (m, 1H), 3.45 (s, 2H), 2.37 (d, 2H, J=9 Hz), 1.89 (br. m, 5H), 1.35 (br. m, 18H), 1.05 (s, 5H), 0.94 (d, 4H, J=2 Hz), 0.89 (d, 6H, J=2 Hz), 0.70 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate

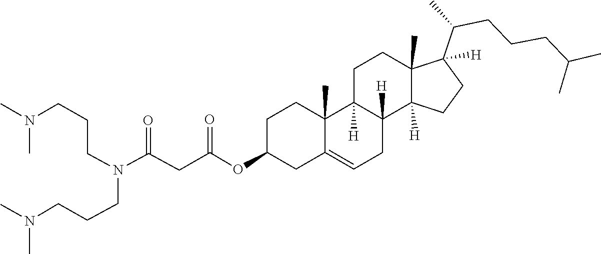

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-3-oxopropanoic acid (3.12 g, 6.61 mmol) in dichloromethane (60 ml) stirring under nitrogen was added tetramethyldipropylenetriamine (2.30 mL, 9.81 mmol), dimethylaminopyridine (0.08 g, 0.65 mmol), and 1-ethyl-3-(3-dimethylaminopropyl)carbodiimide hydrochloride (1.90 g, 9.81 mmol). The reaction mixture was cooled to 0° C. and diisopropylethylamine (3.46 mL, 19.62 mmol) was added dropwise over 20 minutes. The mixture was allowed to gradually warm to room temperature and proceed overnight. The solution was then diluted with dichloromethane, washed with saturated aqueous sodium bicarbonate (1×50 mL) and brine (1×50 mL), dried over sodium sulfate, filtered, and concentrated to an oil. The oil was taken up in dichloromethane and purified on silica with a 0-60% (9:1 methanol/conc. aqueous ammonium hydroxide) gradient in dichloromethane to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate (1.82 g, 2.84 mmol, 43.4%) as a yellow oil. UPLC/ELSD: RT: 1.85 min. MS (ES): m/z (MH⁺) 643.0 for C₄₀H₇₁N₃O₃. ¹H NMR (300 MHz, CDCl₃) δ: ppm 5.39 (m, 1H), 4.67 (m, 1H), 3.54 (s, 2H), 3.35 (br. m, 4H), 2.37 (br. m, 6H), 2.22 (d, 12H, J=3 Hz), 1.50 (br. m, 28H), 1.02 (br. s, 5H), 0.92 (d, 4H, J=6 Hz), 0.88 (d, 6H, J=9 Hz), 0.68 (s, 3H).

### Step 4: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate dihydrochloride

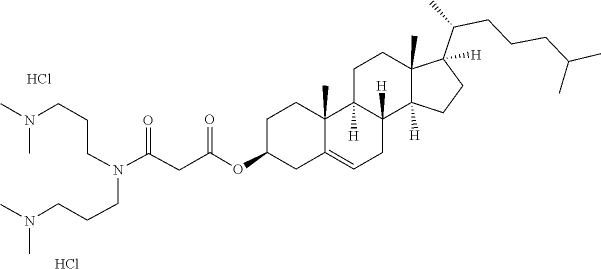

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate (0.22 g, 0.32 mmol) in diethyl ether (4.3 mL) and isopropanol (0.22 mL) was added hydrochloric acid (5.5 M in isopropanol, 0.37 mL, 1.85 mmol) dropwise. The mixture was cooled to 0° C. and stirred vigorously for 30 minutes, after which the white precipitate was filtered out via vacuum filtration and washed repeatedly with cold ether. The residue was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate dihydrochloride as a white waxy solid (0.11 g, 0.15 mmol, 46.6%). UPLC/ELSD: RT: 1.81 min. MS (ES): m/z (MH⁺) 627.99 for C₄₀H₇₃Cl₂N₃O₃. ¹H NMR (300 MHz, CD₃OD) δ: ppm 5.41 (br. s, 1H), 4.64 (br. m, 1H), 3.57 (br. m, 7H), 3.33 (br. s, 2H), 3.20 (br. m, 5H), 2.93 (d, 15H, J=6 Hz), 2.40 (d, 2H, J=9 Hz), 2.05 (br. m, 12H), 1.55 (br. m, 14H), 1.20 (br. m, 13H), 1.07 (s, 7H), 0.98 (d, 5H, J=6 Hz), 0.91 (d, 7H, J=6 Hz), 0.74 (s, 3H).

### R. Compound SA51: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate dihydrochloride

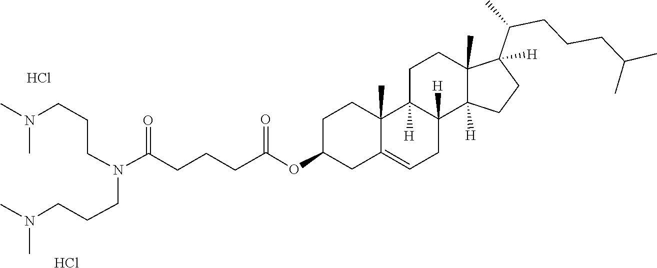

### Step 1: 5-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid

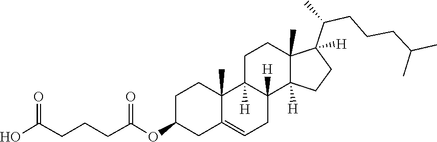

To a solution of cholesterol (5.00 g, 12.67 mmol) in acetone (50 mL) stirring under nitrogen was added glutaric anhydride (2.63 g, 22.81 mmol) and triethylamine (3.21 mL, 22.81 mmol). The reaction mixture was refluxed at 56° C., turning from a white slurry to a colorless clear solution, and allowed to proceed at reflux for 3 days. Following, the solution was cooled to room temperature, concentrated under vacuum, and taken up in 150 mL dichloromethane. This was then washed with 0.5 M HCl (1×100 mL) and saturated aqueous ammonium chloride (1×100 mL), dried over sodium sulfate, filtered, and concentrated to give a white solid. The solid was taken up in dichloromethane and purified on silica with a 0-50% ethyl acetate gradient in hexanes to give 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (6.011 g, 12.00 mmol, 94.7%) as a white solid. UPLC/ELSD: RT: 2.96 min. MS (ES): m/z (MH⁺) 501.7 for C₃₂H₅₂O₄. ¹H NMR (300 MHz, CDCl₃) δ: ppm 5.40 (m, 1H), 4.66 (m, 1H), 2.45 (br. m, 5H), 2.01 (br. m, 3H), 1.85 (br. m, 3H), 1.34 (br. m, 22H), 0.94 (d, 3H, J=6 Hz), 0.88 (d, 6H, J=9 Hz), 0.70 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate

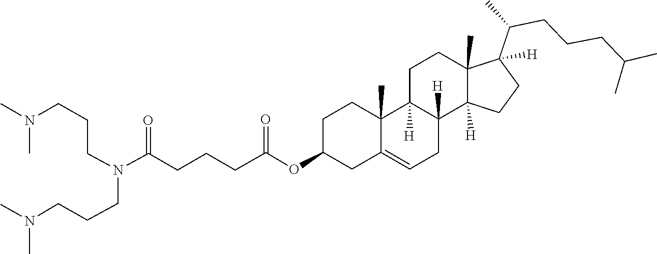

To a solution of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (6.01 g, 11.88 mmol) in dichloromethane (100 mL) stirring under nitrogen was added tetramethyldipropylenetriamine (4.19 mL, 17.82 mmol), dimethylaminopyridine (0.15 g, 1.19 mmol), and 1-ethyl-3-(3-dimethylaminopropyl)carbodiimide hydrochloride (3.45 g, 17.83 mmol). The solution was cooled to 0° C., and then diisopropylethylamine (6.29 mL, 35.65 mmol) was added dropwise. The reaction mixture was allowed to gradually warm to room temperature and proceed overnight. The solution was diluted further with dichloromethane, washed with saturated aqueous sodium bicarbonate (1×100 mL) and brine (1×100 mL), dried over sodium sulfate, filtered, and concentrated to give an oil. The material was taken up in dichloromethane and purified on silica in a 0-60% (9:1 methanol: aqueous ammonium hydroxide) gradient in dichloromethane to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate (2.30 g, 11.88 mmol, 28.9%) as an oil. UPLC/ELSD: RT: 2.02 min. MS (ES): m/z (MH) 671.1 for C₄₂H₇₅N₃O₃. ¹H NMR (300 MHz, CDCl₃) δ: ppm 5.36 (m, 1H), 4.60 (m, 1H), 3.33 (br. m, 5H), 2.39 (br. m, 12H), 2.23 (d, 11H, J=6 Hz), 1.99 (br. m, 4H), 1.84 (br. m, 3H), 1.71 (br. m, 5H), 1.33 (br. m, 11H), 1.14 (br. m, 7H), 1.02 (s, 6H), 0.92 (d, 3H, J=6 Hz), 0.87 (d, 5H, J=9 Hz), 0.68 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate dihydrochloride

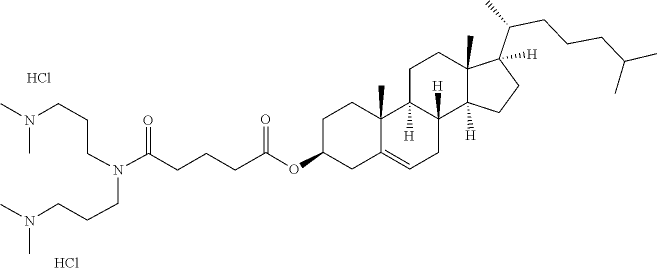

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate (0.23 g, 0.33 mmol) in diethyl ether (4.6 mL) and isopropanol (0.23 mL) was added hydrochloric acid (5.5M in isopropanol, 0.37 mL, 1.85 mmol) dropwise. The mixture was cooled to 0° C. and stirred vigorously for 30 minutes, after which the white precipitate was filtered out via vacuum filtration and washed repeatedly with cold ether. The residue was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate dihydrochloride as a white waxy solid (0.12 g, 0.16 mmol, 49.5%). UPLC/ELSD: RT: 1.86 min. MS (ES): m/z (MH⁺) 671.81 for C₄₂H₇₇Cl₂N₃O₃. ¹H NMR (300 MHz, CD₃OD) δ: ppm 5.41 (br. s, 1H), 4.55 (br. m, 1H), 3.54 (t, 5H, J=6 Hz), 3.24 (br. m, 6H), 2.94 (d, 14H, J=6 Hz), 2.55 (t, 2H, J=6 Hz), 2.43 (t, 2H, J=6 Hz), 2.35 (d, 2H, J=9 Hz), 2.05 (br. m, 6H), 1.90 (br. m, 6H), 1.55 (br. m, 12H), 1.19 (br. m, 10H), 1.07 (s, 7H), 0.98 (d, 4H, J=6 Hz), 0.90 (d, 7H, J=6 Hz), 0.74 (s, 3H).

### S. Compound SA56: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate

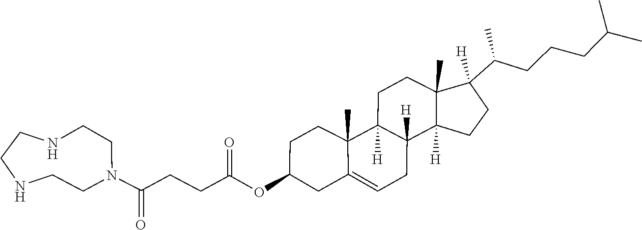

### Step 1: Di-tert-butyl 7-(4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoyl)-1,4,7-triazonane-1,4-dicarboxylate

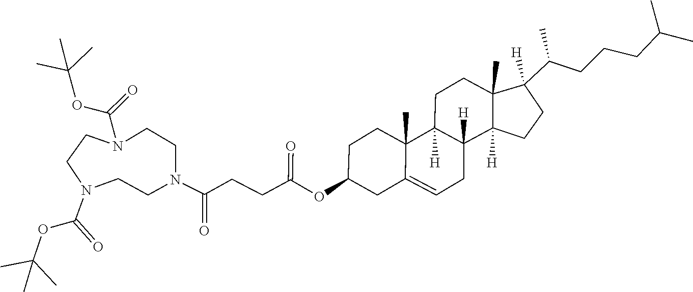

To a stirred solution of cholesteryl hemisuccinate (100 mg, 0.205 mmol), 1,4-di-tert-butyl 1,4,7-triazonane-1,4-dicarboxylate (Enamine, Monmouth Junction, NJ) (0.068 g, 0.20 mmol), and DMAP (cat.) in DCM (1.4 mL) was added 1-ethyl-3-(3-dimethylaminopropyl)carbodiimide hydrochloride (0.060 g, 0.31 mmol). The reaction mixture stirred at rt and was monitored by TLC. At 21.5 h water (1.5 mL) was added. After stirring for 16 h additional water (10 mL) was added. The mixture was then extracted with DCM (2×15 mL). The combined organics were passed through a hydrophobic frit, dried over Na₂SO₄, and then concentrated. The crude material was purified via silica gel chromatography (0-4% MeOH in DCM) to afford di-tert-butyl 7-(4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoyl)-1,4,7-triazonane-1,4-dicarboxylate (130 mg, 0.163 mmol, 79.3%) as a clear oil. UPLC/ELSD: RT=3.41 min. MS (ES): m/z=1619.2 [2M+Na]⁺ for C₄₇H₇₉N₃O7; ¹H NMR (300 MHz, CDCl₃): δ 5.33-5.39 (m, 1H), 4.52-4.68 (m, 1H), 3.18-3.79 (br. m, 12H), 2.49-2.71 (m, 4H), 2.24-2.39 (m, 2H), 1.74-2.06 (br. m, 5H), 0.93-1.71 (br. m, 39H), 1.01 (s, 3H), 0.91 (d, 3H, J=6.5 Hz), 0.87 (d, 3H, J=6.6 Hz), 0.86 (d, 3H, J=6.5 Hz), 0.67 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate dihydrochloride

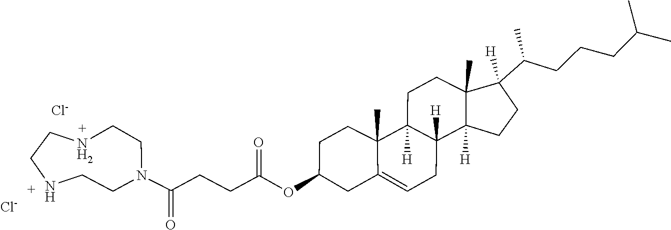

To a solution of di-tert-butyl 7-(4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoyl)-1,4,7-triazonane-1,4-dicarboxylate (123 mg, 0.154 mmol) in iPrOH (2.0 mL) was added 5-6 N HCl in iPrOH (0.18 mL). The reaction mixture stirred at 40° C. and was monitored by LCMS. At 17 h, additional iPrOH (2.0 mL) and 5-6 N HCl in iPrOH (0.06 mL) were added. At 41 h, the reaction mixture was cooled to rt, and ACN (4 mL) was added. Solids were collected by vacuum filtration and rinsed with ACN to afford (3S,8S,9S,10R,13R,14S,17R)-10,13-dimethyl-17-((R)-6-methylheptan-2-yl)-2, 3,4,7,8,9,10,11,12,13,14,15,16,17-tetradecahydro-1H-cyclopenta[a]phenanthren-3-yl 4-oxo-4-(1,4,7-triazonan-1-yl)butanoate dihydrochloride (0.082 g, 0.11 mmol, 73.8%) as a white solid. UPLC/ELSD: RT=2.30 min. MS (ES): m/z=598.1 [M+H]⁺ for C₃₇H₆₃N₃O₃; ¹H NMR (300 MHz, CDCl₃): δ 10.36 (br. s, 2H), 10.11 (br. s, 2H), 5.34-5.43 (m, 1H), 4.50-4.65 (m, 1H), 3.97-4.29 (m, 4H), 3.64-3.95 (m, 6H), 3.41-3.61 (m, 2H), 2.66-2.84 (m, 2H), 2.45-2.65 (m, 2H), 2.21-2.40 (m, 2H), 1.75-2.08 (br. m, 5H), 0.94-1.70 (br. m, 21H), 1.01 (s, 3H), 0.91 (d, 3H, J=6.4 Hz), 0.87 (d, 3H, J=6.5 Hz), 0.86 (d, 3H, J=6.6 Hz), 0.68 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate

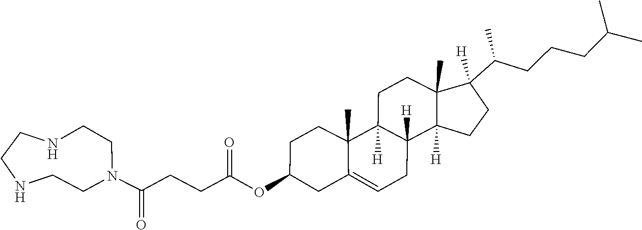

(3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate dihydrochloride (0.054 g, 0.075 mmol) was suspended in 5% aq. NaHCO₃ solution (10 mL) and then extracted with DCM (3×10 mL). K₂CO₃ (ca. 100 mg) was added to the aqueous layer. The aqueous layer was extracted with DCM (2×10 mL). The combined organics were passed through a hydrophobic frit, dried over Na₂SO₄, and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate (0.023 g, 0.037 mmol, 50.0%) as a white solid. ¹H NMR (300 MHz, CDCl₃): δ 5.33-5.40 (m, 1H), 4.54-4.70 (m, 1H), 3.41-3.57 (m, 4H), 2.99-3.15 (m, 4H), 2.72-2.84 (m, 4H), 2.58-2.72 (m, 4H), 2.24-2.39 (m, 2H), 1.74-2.19 (br. m, 7H), 0.94-1.70 (br. m, 21H), 1.01 (s, 3H), 0.91 (d, 3H, J=6.5 Hz), 0.87 (d, 3H, J=6.6 Hz), 0.86 (d, 3H, J=6.6 Hz), 0.67 (s, 3H). UPLC/ELSD: RT=2.39 min. MS (ES): m/z=598.6 [M+H]⁺ for C₃₇H₆₃N₃O₃.

### T. Compound SA57: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 6-(bis(3-(dimethylamino)propyl)amino)-6-oxohexanoate

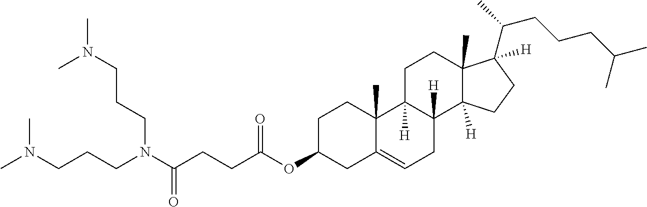

### Step 1: 6-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-6-oxohexanoic acid

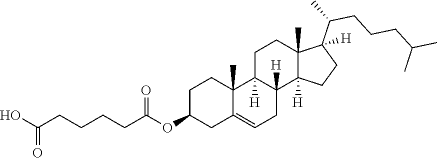

To a solution of cholesterol (5.00 g, 12.93 mmol) in dichloromethane (50 mL) stirring under nitrogen was added adipic anhydride (1.66 g, 12.93 mmol). Then, pyridine (3.97 mL, 28.45 mmol) was added dropwise over 10 minutes. The reaction mixture was heated to a reflux at 40° C. and proceeded overnight. Then, the mixture was allowed to cool to room temperature and concentrated to a yellow oil. The oil was taken up in dichloromethane and purified on silica without additional workup in a 0-30% ethyl acetate gradient in hexanes to give 6-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-6-oxohexanoic acid (1.97 g, 3.83 mmol, 29.6%) as a white solid. UPLC/ELSD: RT: 3.09 min. MS (ES): m/z (MH⁺) 515.7 for C₃₃H₅₄O₄. ¹H NMR (300 MHz, CDCl₃) δ: ppm 12.15 (br. s, 1H), 5.39 (m, 1H), 4.63 (br. m, 1H), 2.40 (br. m, 6H), 2.00 (br. m, 2H), 1.85 (br. m, 3H), 1.70 (br. m, 4H), 1.34 (br. m, 19H), 1.03 (s, 6H), 0.93 (d, 4H, J=6 Hz), 0.88 (d, 6H, J=6 Hz), 0.69 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 6-(bis(3-(dimethylamino)propyl)amino)-6-oxohexanoate

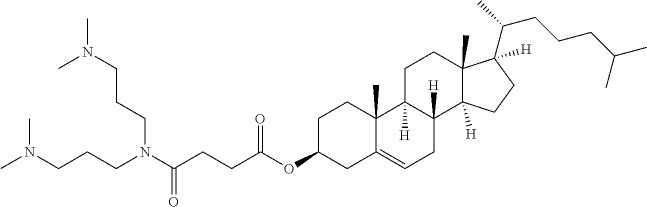

To a solution of 6-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-6-oxohexanoic acid (1.00 g, 1.92 mmol) in dichloromethane (25 mL) stirring under nitrogen was added tetramethyldipropylenetriamine (0.68 mL, 2.89 mmol), dimethylaminopyridine (0.02 g, 0.19 mmol), and 1-ethyl-3-(3-dimethylaminopropyl)carbodiimide hydrochloride (0.56 g, 2.89 mmol). The resulting solution was cooled to 0° C. and diisopropylethylamine (1.02 mL, 5.77 mmol) was added dropwise. The mixture was allowed to gradually warm to room temperature and proceed overnight. Then, the solution was diluted with dichloromethane, washed with saturated aqueous sodium bicarbonate (1×10 mL) and brine (1×10 mL), dried over sodium sulfate, filtered, and concentrated to an oil. The oil was taken up in DCM and purified on silica with a 0-60% (8:2:0.1 dichloromethane/methanol/conc. aqueous ammonium hydroxide) gradient in dichloromethane to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-(bis(3-(dimethylamino)propyl)amino)-6-oxohexanoate as a yellow oil by ¹H NMR, so the material was purified again on silica using a 0-25% (8:2:0.1 dichloromethane/methanol/conc. aqueous ammonium hydroxide) gradient in dichloromethane to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-(bis(3-(dimethylamino)propyl)amino)-6-oxohexanoate as a light yellow oil (0.38 g, 0.54 mmol, 28.2%). UPLC/ELSD: RT: 2.11 min. MS (ES): m/z (MH⁺) 685.1 for C₄₃H₇₇N₃O₃. ¹H NMR (300 MHz, CDCl₃) δ: ppm 5.40 (m, 3H), 5.20 (m, 1H), 4.40 (br. m, 1H), 3.22 (m, 4.48), 2.58 (s, 3H), 2.38 (t, 2H, J=9 Hz), 2.26 (s, 6H), 2.20 (d, 3H, J=9 Hz), 2.14 (br. s, 9H), 1.49 (br. m, 24H), 0.95 (br. m, 7H), 0.85 (s, 5H), 0.75 (d, 4H, J=6 Hz), 0.70 (d, 5H, J=9 Hz), 0.51 (s, 3H).

### U. Compound SA58: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride

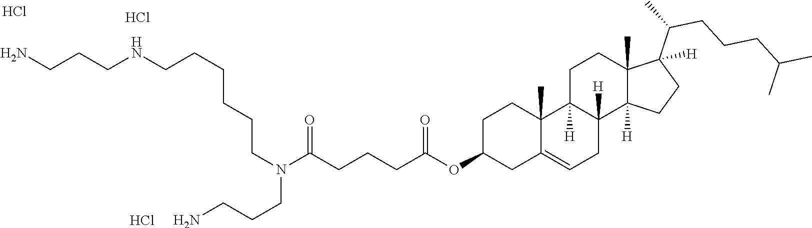

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate

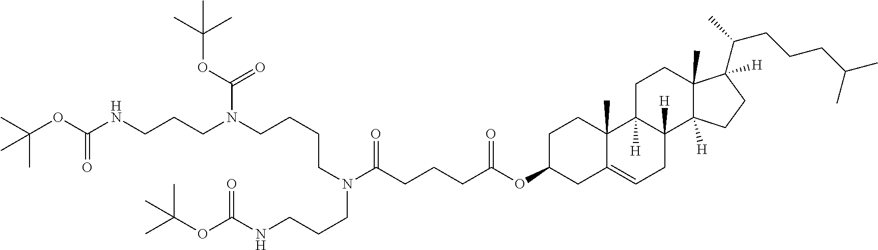

To a solution of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (0.76 g, 1.49 mmol) in dichloromethane (20 mL) stirring under nitrogen was added tert-butyl (3-((tert-butoxycarbonyl)amino)propyl)(4-((3-((tert-butoxycarbonyl)amino)propyl)amino)butyl)carbamate (0.75 g, 1.49 mmol), dimethylaminopyridine (0.02 g, 0.15 mmol), and 1-ethyl-3-(3-dimethylaminopropyl)carbodiimide hydrochloride (0.43 g, 2.24 mmol). The resulting solution was cooled to 0° C. and diisopropylethylamine (0.79 mL, 4.48 mmol) was added dropwise. The mixture was allowed to gradually warm to room temperature and proceed overnight. Then, the solution was diluted with dichloromethane, washed with saturated aqueous sodium bicarbonate (1×10 mL) and brine (1×10 mL), dried over sodium sulfate, filtered, and concentrated to an oil. The oil was taken up in DCM and purified on silica with a 0-60% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate as a light yellow oil (0.71 g, 0.72 mmol, 48.2%). UPLC/ELSD: RT: 3.37 min. MS (ES): m/z (MH⁺) 986.4 for C₅₇H₁₀₀N₄O₉. ¹H NMR (300 MHz, CDCl₃) δ: ppm 5.39 (m, 2H), 4.64 (br. m, 1H), 3.27 (br. m, 11H), 2.38 (br. m, 6H), 1.86 (br. m, 13H), 1.46 (br. d, 32H), 1.15 (br. m, 11H), 1.03 (s, 5H), 0.94 (d, 3H, J=9 Hz), 0.88 (d, 5H, J=9 Hz), 0.70 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride

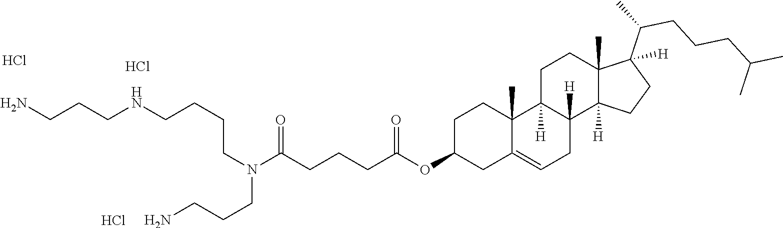

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate (0.71 g, 0.72 mmol) in 2-propanol (10 mL) stirring under nitrogen was added hydrochloric acid (5.5 M in 2-propanol, 1.44 mL, 7.20 mmol) dropwise. The mixture was heated to 45° C. and allowed to stir overnight. Then, the solution was cooled to room temperature, and acetonitrile (5 mL) was added to the mixture. It was then sonicated to remove precipitated solid off the side of the flask. After stirring for 30 minutes after sonication, the solid was filtered out by vacuum filtration, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride as a light purple solid (0.39 g, 0.47 mmol, 65.6%). UPLC/ELSD: RT: 1.68 min. MS (ES): m/z (MH⁺) 686.1 for C₄₂H₇₉Cl₃N₄O₃. ¹H NMR (300 MHz, CD₃OD) δ: ppm 5.40 (s, 1H), 4.90 (br. s, 9H), 4.55 (br. s, 1H), 3.33 (br. m, 12H), 2.32 (br. 6H), 2.16 (br. m, 2H), 2.05 (s, 5H), 1.91 (br. m, 10H), 1.54 (br. m, 7H), 1.39 (br. m, 4H), 1.17 (d, 8H, J=6 Hz), 1.06 (s, 5H), 0.97 (d, 4H, J=6 Hz), 0.90 (d, 6H, J=6 Hz), 0.73 (s, 3H).

### V. Compound SA59: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-3-oxopropanoate trihydrochloride

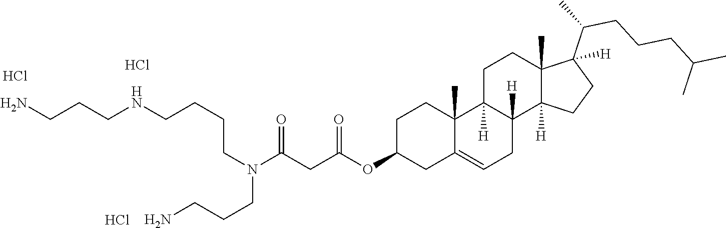

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2, 2-dimethyl-4,15-dioxo-3-oxa-5,9,14-triazaheptadecan-17-oate

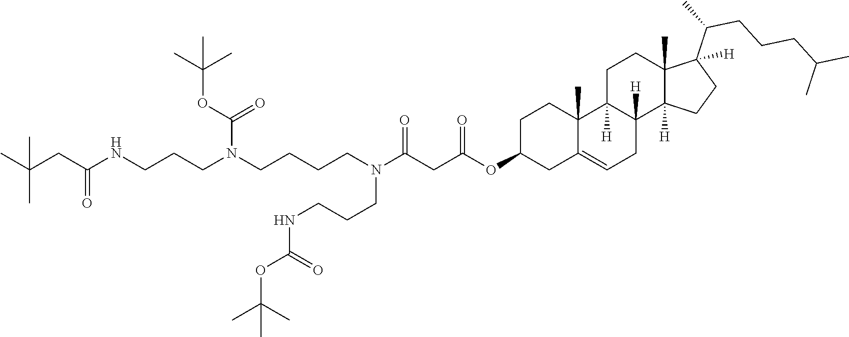

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-3-oxopropanoic acid (0.70 g, 1.47 mmol) in dichloromethane (20 ml) stirring under nitrogen was added tert-butyl (3-((tert-butoxycarbonyl)amino)propyl)(4-((3-((tert-butoxycarbonyl)amino)propyl)amino)butyl)carbamate (0.74 g, 1.47 mmol), dimethylaminopyridine (0.02 g, 0.15 mmol), and 1-ethyl-3-(3-dimethylaminopropyl)carbodiimide hydrochloride (0.43 g, 2.20 mmol). The resulting solution was cooled to 0° C., and diisopropylethylamine (0.78 mL, 4.40 mmol) was added dropwise. The mixture was allowed to gradually warm to room temperature and proceed overnight. Then, the solution was diluted with dichloromethane, washed with water (3×20 mL), dried over sodium sulfate, filtered, and concentrated to an oil. The oil was taken up in DCM and purified on silica with a 0-60% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaheptadecan-17-oate as a light yellow oil (1.13 g, 1.18 mmol, 80.4%). UPLC/ELSD: RT: 3.29 min. MS (ES): m/z (MH⁺) 958.4 for C₅₅H₉₆N₄O₉. ¹H NMR (300 MHz, CDCl₃) δ: ppm 5.39 (m, 2H). 4.70 (br. m, 2H), 3.26 (br. m, 13H), 2.37 (d, 2H, J=6 Hz), 1.86 (br. m, 16H), 1.45 (br. s, 28H), 1.23 (br. m, 12H), 1.03 (s, 4H), 0.94 (d, 3H, J=6 Hz), 0.88 (d, 5H, J=6 Hz). 0.69 (s, 3H).

### Step 2: (3S,8S,9S,10R,13R,14S,17R)-10,13-Dimethyl-7-((R)-6-methylheptan-2-yl)-2,3,4, 7, 8,9,10,11,12,13,14,15,16,17-tetradecahydro-1H-cyclopenta[a]phenanthren-3-yl 3-((3-aminopropyl)(4-((3-aminopropyl)amino)butyl)amino)-3-oxopropanoate trihydrochloride

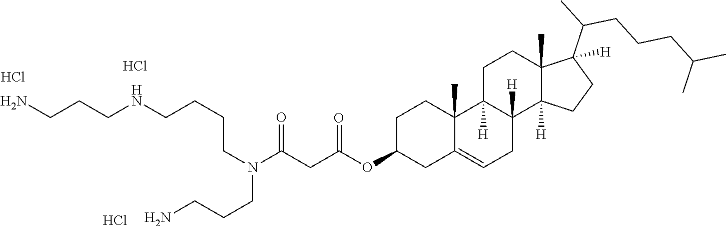

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaheptadecan-17-oate (1.13 g, 1.18 mmol) in 2-propanol (15 mL) stirring under nitrogen was added hydrochloric acid (5.5 M in 2-propanol, 2.36 mL, 11.79 mmol) dropwise. The mixture was heated to 40° C. and allowed to proceed overnight. Then, acetonitrile (5 mL) was added, and the solution was sonicated until all solid was displaced from the sides of the flask. After 30 minutes of stirring after sonication, the solid was filtered out by vacuum filtration and washed repeatedly with acetonitrile and dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-3-oxopropanoate trihydrochloride as a light purple solid (0.52 g, 0.64 mmol, 54.4%). UPLC/ELSD: RT: 1.57 min. MS (ES): m/z (MH⁺) 657.2 for C₄₀H₇₅N₄O₃. ¹H NMR (300 MHz, CD₃OD) δ: ppm 5.42 (m, 1H), 4.88 (br. s, 11H), 4.60 (m, 1H), 3.33 (br. m, 16H), 2.39 (d, 2H, J=3 Hz), 1.55 (br. m, 40H), 0.96 (d, 4H, J=6 Hz), 0.90 (d, 6H, J=6 Hz), 0.74 (s, 3H).

### W. Compound SA60: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride

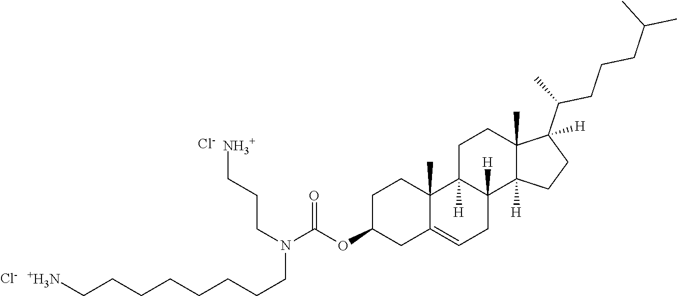

### Step 1: tert-Butyl N-{8-[(2-cyanoethyl)amino]octyl}carbamate

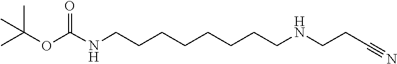 [SMILES: CC(C)(C)OC(=O)NCCCCCCCCNCCC#N]

To a foil-covered, stirred suspension of tert-butyl N-(8-aminooctyl)carbamate (2.50 g, 10.2 mmol) in water (100 mL) was added acrylonitrile (1.00 mL, 15.3 mmol). The suspension stirred at rt and was monitored by TLC. At 26 h, the reaction mixture was diluted with 5% aq. NaHCO₃ solution (200 mL) and then extracted with EtOAc (3×100 mL). The combined organics were washed with brine, dried over Na₂SO₄, and concentrated. The crude material was purified via silica gel chromatography (0-10% MeOH in DCM) to afford tert-butyl N-{8-[(2-cyanoethyl)amino]octyl}carbamate (1.833 g, 6.163 mmol, 60.2%) as a yellow oil. UPLC/ELSD: RT=0.28 min. MS (ES): m/z=197.9 [(M+H) ˜(CH₃)₂C═CH₂—CO₂]⁺ C₁₆H₃₁N₃O₂; ¹H NMR (300 MHz, CDCl₃): δ 4.50 (br. s, 1H), 3.09 (dt, 2H, 6.6, 6.5 Hz), 2.92 (t, 2H, J=6.6 Hz), 2.62 (t, 2H, J=7.1 Hz), 2.51 (t, 2H, J=6.7 Hz), 1.18-1.58 (m, 13H), 1.44 (s, 9H).

### Step 2: tert-Butyl N-{8-[benzyl(2-cyanoethyl)amino]octyl}carbamate

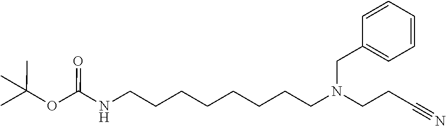 [SMILES: CC(C)(C)OC(=O)NCCCCCCCCN(CCC#N)Cc1ccccc1]

A mixture of tert-butyl N—{8-[(2-cyanoethyl)amino]octyl}carbamate (0.870 g, 2.92 mmol), potassium carbonate (0.808 g, 5.85 mmol), benzyl bromide (0.40 mL, 3.4 mmol), and potassium iodide (0.097 g, 0.58 mmol) in ACN (17.5 mL) was stirred at 65° C. The reaction was monitored by LCMS. At 3 h, the reaction mixture was cooled to rt, filtered through a pad of Celite, rinsed with MTBE, and concentrated. The residue was taken up in 5% aq. NaHCO₃ solution (50 mL) and then extracted with MTBE (3×30 mL). The combined organics were washed with brine, dried over Na₂SO₄, and concentrated. The crude material was purified via silica gel chromatography (0-40% EtOAc in hexanes) to afford tert-butyl N-{8-[benzyl(2-cyanoethyl)amino]octyl}carbamate (0.783 g, 2.02 mmol, 69.1%) as a clear oil. UPLC/ELSD: RT=0.44 min. MS (ES): m/z=331.9 [(M+H)—(CH₃)₂C═CH₂]⁺ for C₂₃H₃₇N₃O₂. ¹H NMR (300 MHz, CDCl₃): δ 7.21-7.39 (m, 5H), 4.49 (br. s, 1H), 3.60 (s, 2H), 3.09 (dt, 2H, J=6.5, 6.2 Hz), 2.78 (t, 2H, J=6.9 Hz), 2.48 (t, 2H, J=7.4 Hz), 2.39 (t, 2H, J=7.0 Hz), 1.38-1.54 (m, 4H), 1.44 (s, 9H), 1.19-1.36 (m, 8H).

### Step 3: tert-Butyl N-{3-[benzyl({8-[(tert-butoxycarbonyl)amino]octyl})amino]propyl}carbamate

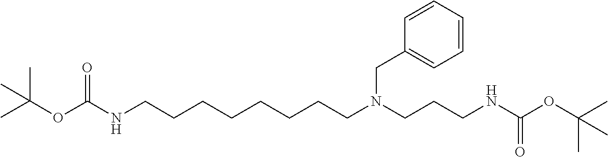 [SMILES: CC(C)(C)OC(=O)NCCCCCCCCN(CCCNC(=O)OC(C)(C)C)Cc1ccccc1]

To a stirred solution of tert-butyl N—{8-[benzyl(2-cyanoethyl)amino]octyl}carbamate (0.492 g, 1.27 mmol) in MeOH (8.8 mL) was added di-tert-butyl dicarbonate (0.693 g, 3.17 mmol) and nickel(II) chloride hexahydrate (0.030 g, 0.13 mmol). The reaction mixture was cooled to 0° C. in an ice bath and then NaBH₄ (0.336 g, 8.89 mmol) was added portionwise over 30 min to give a black suspension (CAUTION: VIGOROUS GAS EVOLUTION OCCURS DURING ADDITION). The reaction mixture stirred at rt and was monitored by LCMS. At 17.3 h, diethylenetriamine (0.15 mL, 1.4 mmol) was added dropwise, and the reaction mixture stirred at rt. After 30 min, additional diethylenetriamine (0.15 mL) was added. After 1.5 h, the reaction mixture was concentrated, taken up in 5% aq. NaHCO₃ solution and extracted with EtOAc (3×). The combined organics were washed with 5% aq. NaHCO₃ solution and brine, dried over Na₂SO₄, and concentrated. The crude material was purified via silica gel chromatography (20-65% EtOAc in hexanes) to afford tert-butyl N—{3-[benzyl({8-[(tert-butoxycarbonyl)amino]octyl})amino]propyl}carbamate (0.512 g, 1.04 mmol, 82.0%) as a clear oil. UPLC/ELSD: RT=0.92 min. MS (ES): m/z=492.5 [M+H]⁺ for C₂₈H₄₉N₃O₄; ¹H NMR (300 MHz, CDCl₃): δ 7.19-7.35 (m, 5H), 5.52 (br. s, 1H), 4.49 (br. s, 1H), 3.51 (s, 2H), 3.00-3.22 (m, 4H), 2.46 (t, 2H, J=6.2 Hz), 2.36 (t, 2H, J=7.4 Hz), 1.56-1.68 (m, 2H), 1.36-1.55 (m, 22H), 1.18-1.33 (m, 8H).

### Step 4: tert-Butyl N-[3-({8-[(tert-butoxycarbonyl)amino]octyl}amino)propyl]carbamate

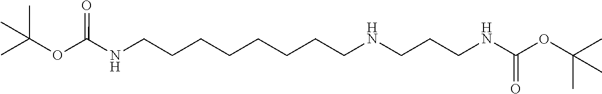 [SMILES: CC(C)(C)OC(=O)NCCCCCCCCNCCCNC(=O)OC(C)(C)C]

A solution of tert-butyl N—{3-[benzyl({8-[(tert-butoxycarbonyl)amino]octyl})amino]propyl}carbamate (0.496 g, 1.01 mmol) and 10% Pd/C (0.429 g, 0.202 mmol) in ethanol (10 mL) was stirred under a balloon of H₂. The reaction was monitored by TLC. At 3 h, the reaction mixture was diluted with EtOAc (20 mL), filtered through a pad of Celite, and rinsed with EtOAc. The filtrate was concentrated, taken up in EtOAc, and filtered using a 0.45 μm syringe filter. Filtered organics were concentrated to afford tert-butyl N-[3-({8-[(tert-butoxycarbonyl)amino]octyl}amino)propyl]carbamate (0.323 g, 0.805 mmol, 79.8%) as an off-white solid). UPLC/ELSD: RT=0.59 min. MS (ES): m/z=402.0 [M+H]⁺ for C₂₁H₄₃N₃O₄; ¹H NMR (300 MHz, CDCl₃): δ 5.17 (br. s, 1H), 4.50 (br. s, 1H), 3.20 (dt, 2H, J=6.0, 6.0 Hz), 3.09 (dt, 2H, J=6.5, 6.4 Hz), 2.67 (t, 2H, J=6.6 Hz), 2.58 (t, 2H, J=7.1 Hz), 1.89 (br. s, 1H), 1.59-1.74 (m, 2H), 1.37-1.55 (m, 22H), 1.21-1.37 (m, 8H).

### Step 5: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate

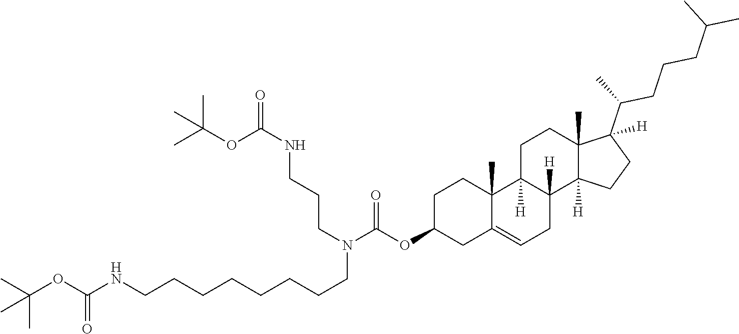

Cholesterol 4-nitrophenyl carbonate (0.300 g, 0.544 mmol), tert-butyl N-[3-({8-[(tert-butoxycarbonyl)amino]octyl}amino)propyl]carbamate (0.240 g, 0.598 mmol), and triethylamine (0.12 mL, 0.85 mmol) were combined in CHCl₃ (4.8 mL). The reaction mixture stirred at 50° C. and was monitored by TLC. At 20.25 h, tert-butyl N-[3-({8-[(tert-butoxycarbonyl)amino]octyl}amino)propyl]carbamate (77 mg) and triethylamine (0.04 mL) were added. The reaction mixture stirred at 60° C. At 95 h, the reaction mixture was cooled to rt, diluted with DCM (20 mL), and washed with water (25 mL). The aqueous layer was extracted with DCM (2×20 mL). The combined organics were passed through a hydrophobic frit, dried over Na₂SO₄, and concentrated. The crude material was purified via silica gel chromatography (0-30% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate (0.398 g, 0.489 mmol, 89.9%) as a clear oil. UPLC/ELSD: RT=3.47 min. MS (ES): m/z=836.5 [M+Na]⁺ for C₄₉H₅₇N₃O₆; ¹H NMR (300 MHz, CDCl₃): δ 5.22-5.43 (m, 2H), 4.40-4.84 (m, 2H), 3.00-3.39 (br. m, 8H), 2.21-2.44 (m, 2H), 1.73-2.07 (br. m, 5H), 0.93-1.71 (br. m, 53H), 1.02 (s, 3H), 0.91 (d, 3H, J=6.4 Hz), 0.86 (d, 3H, J=6.6 Hz), 0.86 (d, 3H, J=6.6 Hz), 0.67 (s, 3H).

### Step 6: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride

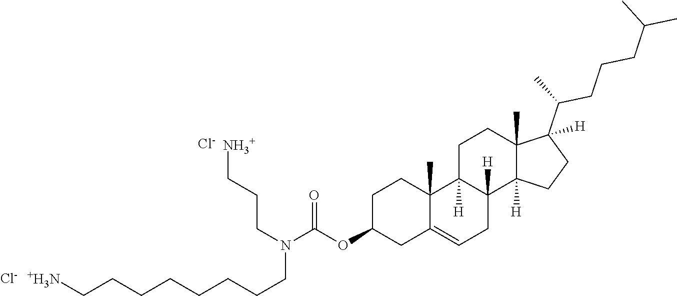

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate (0.395 g, 0.485 mmol) in iPrOH (2.5 mL) was added 5-6 N HCl in iPrOH (0.7 mL). The reaction mixture stirred at 40° C. and was monitored by LCMS. At 17.5 h, the reaction mixture was cooled to rt. ACN (5 mL) was added, the suspension was stirred for 15 min, and the solids were collected by vacuum filtration rinsing with 2:1 ACN:iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride (0.249 g, 0.356 mmol, 73.4%) as a white solid. UPLC/ELSD: RT=1.97 min. MS (ES): m/z=614.4 [M+H]⁺ for C₃₉H₇₃Cl₂N₃O₂; ¹H NMR (300 MHz, CDCl₃): δ 8.00-8.64 (br. m, 6H), 5.33-5.44 (m, 1H), 4.39-4.56 (m, 1H), 2.93-3.54 (br. m, 8H), 2.20-2.43 (m, 2H), 1.69-2.16 (br. m, 10H), 0.93-1.66 (br. m, 30H), 1.02 (s, 3H), 0.91 (d, 3H, J=6.3 Hz), 0.87 (d, 3H, J=6.5 Hz), 0.86 (d, 3H, J=6.5 Hz), 0.68 (s, 3H).

### X. Compound SA61: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-aminopropoxy)butyl)(3-aminopropyl)carbamate dihydrochloride

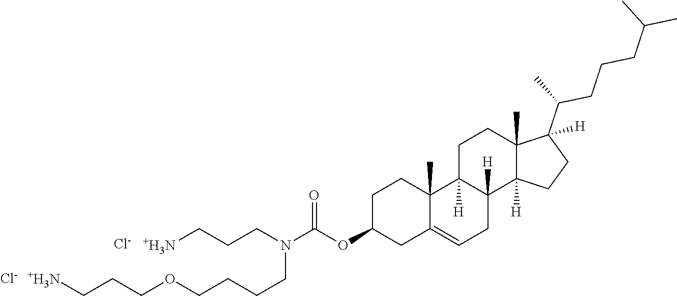

### Step 1: 3-(4-Hydroxybutoxy)propanenitrile

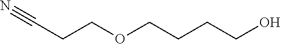

To a solution of 1,4-butanediol (8.0 mL, 91 mmol) and benzyltrimethylammonium hydroxide (0.20 mL, 1.3 mmol, 40 wt % in water) was added acrylonitrile (3.0 mL, 46 mmol). The reaction mixture stirred at rt while covered in foil and was monitored by TLC. At 2 h, the reaction mixture was diluted with water (150 mL) and extracted with 1:1 hexanes/MTBE (50 mL) and EtOAc (3×50 mL). The combined organics were washed with water and brine, dried over MgSO₄, and concentrated. The crude material was purified via silica gel chromatography (50-100% EtOAc in hexanes) to afford 3-(4-hydroxybutoxy)propanenitrile (1.374 g, 9.596 mmol, 21.0%) as a yellow oil. UPLC/ELSD: RT=0.20 min. ¹H NMR (300 MHz, CDCl₃): δ 3.67 (t, 2H, J=6.0 Hz), 3.66 (t, 2H, J=6.4 Hz), 3.50-3.57 (m, 2H), 2.60 (t, 2H, 6.4 Hz), 1.60-1.76 (m, 5H).

### Step 2: 4-(2-Cyanoethoxy)butyl methanesulfonate

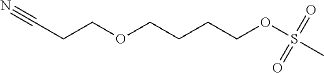

A stirred solution of 3-(4-hydroxybutoxy)propanenitrile (1.00 g, 6.98 mmol) and triethylamine (1.5 mL, 11 mmol) in DCM (10 mL) was cooled to 0° C. in an ice bath, and then methanesulfonyl chloride (0.60 mL, 7.8 mmol) was added dropwise. The reaction was monitored by TLC. The reaction mixture was allowed to slowly come to rt. At 2 h, the reaction mixture was cooled to 0° C. in an ice bath, and additional methanesulfonyl chloride (0.06 mL) was added. At 2 h 10 min, water (10 mL) was added, and the reaction mixture stirred at rt for 5 min. After this time, a 5% aq. NaHCO₃ solution (50 mL) was added, and then the reaction mixture was extracted with DCM (3×30 mL). The combined organics were washed with water and brine, dried over MgSO₄, and concentrated to afford 4-(2-cyanoethoxy)butyl methanesulfonate (1.556 g, 7.032 mmol, quant.) as a yellow oil. The material was carried forward without further purification into the next step. ¹H NMR (300 MHz, CDCl₃): δ 4.28 (t, 2H, J=6.4 Hz), 3.64 (t, 2H, J=6.2 Hz), 3.54 (t, 2H, J=5.9 Hz), 3.01 (s, 3H), 2.59 (t, 2H, J=6.2 Hz), 1.81-1.93 (m, 2H), 1.66-1.78 (m, 2H).

### Step 3: tert-Butyl N-(3-[[4-(2-cyanoethoxy)butyl]amino]propyl)carbamate

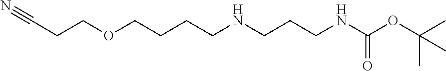 [SMILES: CC(C)(C)OC(=O)NCCCNCCCCOCCC#N]

A solution of tert-butyl N-(3-aminopropyl)carbamate (4.272 g, 24.52 mmol), 4-(2-cyanoethoxy)butyl methanesulfonate (1.550 g, 7.005 mmol), and EtOH (16 mL) was stirred at 65° C. The reaction was monitored by TLC. At 4 h, the reaction mixture was cooled to rt. At 21.5 h, the reaction mixture was concentrated and then taken up in a mixture of EtOAc (75 mL) and water (75 mL). The layers were separated, and the aqueous was extracted with EtOAc (50 mL). The combined organics were washed with water (3×) and brine, dried over Na₂SO₄, and concentrated. The crude material was purified via silica gel chromatography (0-20% (5% conc. aq. NH₄OH in MeOH) in DCM) to afford tert-butyl N-(3-{[4-(2-cyanoethoxy)butyl]amino}propyl)carbamate (1.396 g, 4.662 mmol, 66.6%) as a yellow oil. UPLC/ELSD: RT=0.22 min. MS (ES): m/z=243.8 [(M+H)−t-Bu]⁺ for C₁₅H₂₉N₃O₃; ¹H NMR (300 MHz, CDCl₃): δ 5.19 (br. s, 1H), 3.64 (t, 2H, J=6.3 Hz), 3.50 (t, 2H, J=6.0 Hz), 3.20 (dt, 2H, J=6.2, 5.9 Hz), 2.67 (t, 2H, J=6.6 Hz), 2.61 (t, 2H, J=6.7 Hz), 2.59 (t, 2H, J=6.4 Hz), 1.48-1.70 (m, 6H), 1.44 (s, 9H), 1.10 (br. s, 1H).

### Step 4: tert-Butyl N-(3-[benzyl[4-(2-cyanoethoxy)butyl]amino]propyl)carbamate

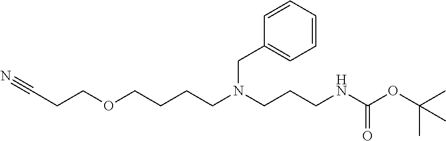 [SMILES: CC(C)(C)OC(=O)NCCCN(CCCCOCCC#N)Cc1ccccc1]

To a mixture of tert-butyl N-(3-{[4-(2-cyanoethoxy)butyl]amino}propyl)carbamate (1.380 g, 4.609 mmol), potassium carbonate (1.274 g, 9.218 mmol), and potassium iodide (0.150 g, 0.904 mmol) in ACN (20 mL) was added benzyl bromide (0.63 mL, 5.3 mmol). The reaction mixture stirred at 65° C. and was monitored by TLC. At 2.5 h, the reaction mixture was cooled to rt and filtered through a pad of Celite rinsing with ACN, and the filtrate was concentrated. The residue was taken up in 5% aq. NaHCO₃ solution (ca. 50 mL), then extracted with MTBE (2×25 mL), and EtOAc (25 mL). The combined organics were washed with brine, dried over Na₂SO₄, and concentrated. The crude material was purified via silica gel chromatography (30-70% EtOAc in hexanes) to afford tert-butyl N-(3-{benzyl[4-(2-cyanoethoxy)butyl]amino}propyl)carbamate (1.372 g, 3.522 mmol, 76.4%) as a yellow oil. UPLC/ELSD: RT=0.29 min. MS (ES): m/z=390.0 [M+H]⁺ for C₂₂H₃₅N₃O₃. ¹H NMR (300 MHz, CDCl₃): δ 7.20-7.39 (m, 5H), 5.41 (br. s, 1H), 3.60 (t, 2H, J=6.4 Hz), 3.51 (s, 2H), 3.38-3.47 (m, 2H), 3.15 (dt, 2H, J=5.8, 5.6 Hz), 2.56 (t, 2H, J=6.4 Hz), 2.47 (t, 2H, J=6.3 Hz), 2.35-2.43 (m, 2H), 1.51-1.69 (m, 6H), 1.44 (s, 9H).

### Step 5: tert-Butyl N-{3-[benzyl(4-{3-[(tert-butoxycarbonyl)amino]propoxy}butyl)amino]propyl}carbamate

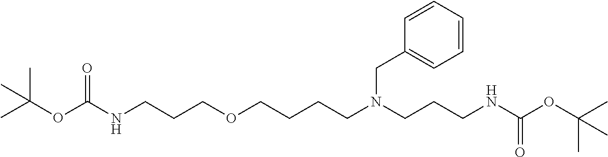 [SMILES: CC(C)(C)OC(=O)NCCCOCCCCN(CCCNC(=O)OC(C)(C)C)Cc1ccccc1]

To a stirred solution of tert-butyl N-(3-{benzyl[4-(2-cyanoethoxy)butyl]amino}propyl)carbamate (1.357 g, 3.484 mmol) in MeOH (23 mL) was added di-tert-butyl dicarbonate (1.901 g, 8.709 mmol) and nickel(II) chloride hexahydrate (0.083 g, 0.35 mmol). The reaction mixture was cooled to 0° C. in an ice bath, and then NaBH₄ (0.923 g, 24.4 mmol) was added portion wise over 40 min (CAUTION: VIGOROUS GAS EVOLUTION OCCURS DURING ADDITION). The reaction mixture stirred at rt and was monitored by LCMS. At 17.25 h, the reaction mixture was cooled to 0° C. in an ice bath, and then NaBH₄ (500 mg) was added portion wise over 30 min. The reaction mixture stirred at rt. At 18.5 h, the reaction mixture was cooled to 0° C. in an ice bath, and then NaBH₄ (100 mg) was added. The reaction mixture stirred at 0° C. At 19.5 h, NaBH₄ (101 mg) was added. At 20.5 h, NaBH₄ (102 mg) was added. At 21.5 h, Boc₂O (850 mg) and NaBH₄ (103 mg) were added. The reaction mixture was allowed to slowly come to rt. At 40.5 h, diethylenetriamine (0.55 mL, 5.1 mmol) was added dropwise, and the reaction mixture stirred at rt for 1 h. After this time, the reaction mixture was concentrated, taken up in 5% aq. NaHCO₃ solution, and extracted with DCM (3×). The biphasic mixture was concentrated to remove volatile organics, and then the mixture was extracted with MTBE (3×). The combined organics were washed with brine, dried over Na₂SO₄, and concentrated. The crude material was purified via silica gel chromatography (0-7% (5% conc. aq. NH₄OH in MeOH) in DCM) to afford tert-butyl N-{3-[benzyl(4-{3-[(tert-butoxycarbonyl)amino]propoxy}butyl)amino]propyl}carbamate (0.836 g, 1.69 mmol, 48.6%) as a light yellow oil. UPLC/ELSD: RT=0.65 min. MS (ES): m/z=494.5 [M+H]⁺ for C₂₇H₄₇N₃O₅; ¹H NMR (300 MHz, CDCl₃): δ 7.19-7.38 (m, 5H), 5.43 (br. s, 1H), 4.87 (br. s, 1H), 3.51 (s, 2H), 3.43 (t, 2H, J=5.9 Hz), 3.31-3.39 (m, 2H), 3.06-3.26 (m, 4H), 2.46 (t, 2H, J=6.2 Hz), 2.35-2.43 (m, 2H), 1.49-1.79 (br. m, 8H), 1.44 (s, 18H).

### Step 6: tert-Butyl N-{3-[4-({3-[(tert-butoxycarbonyl)amino]propyl}amino)butoxy]propyl}carbamate

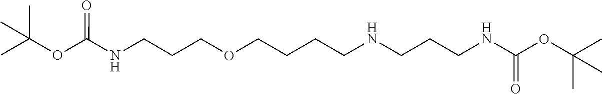 [SMILES: CC(C)(C)OC(=O)NCCCNCCCCOCCCNC(=O)OC(C)(C)C]

A solution of tert-butyl N-{3-[benzyl(4-{3-[(tert-butoxycarbonyl)amino]propoxy}butyl)amino]propyl}carbamate (0.825 g, 1.67 mmol) and 10% Pd/C (0.711 g, 0.334 mmol) in EtOH (10 mL) was stirred under a balloon of H₂. The reaction was monitored by TLC. At 18 h, the reaction mixture was diluted with EtOAc (40 mL) and then filtered through a pad of Celite rinsing with EtOAc. The filtrate was concentrated, taken up in EtOAc, and filtered using a 0.45 μm syringe filter. Filtered organics were concentrated to afford tert-butyl N-{3-[4-({3-[(tert-butoxycarbonyl)amino]propyl}amino)butoxy]propyl}carbamate (0.636 g, 1.58 mmol, 94.3%) as a yellow oil. UPLC/ELSD: RT=0.40 min. MS (ES): m/z=404.5 [M+H]⁺ for C₂₀H₄₁N₃O₅; ¹H NMR (300 MHz, CDCl₃): δ 5.20 (br. s, 1H), 4.91 (br. s, 1H), 3.47 (t, 2H, J=5.9 Hz), 3.41 (t, 2H, J=6.1 Hz), 3.13-3.23 (m, 4H), 2.67 (t, 2H, J=6.6 Hz), 2.61 (t, 2H, J=6.6 Hz), 1.48-1.80 (br. m, 9H), 1.44 (s, 18H).

### Step 7: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-((tert-butoxycarbonyl)amino)propoxy)butyl)(3-((tert-butoxycarbonyl)amino)propyl)carbamate

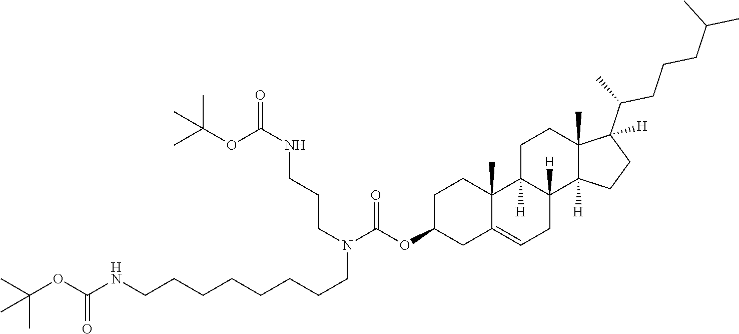

A solution of cholesterol 4-nitrophenyl carbonate (0.663 g, 1.20 mmol), tert-butyl N-{3-[4-({3-[(tert-butoxycarbonyl)amino]propyl}amino)butoxy]propyl}carbamate (0.630 g, 1.56 mmol), and triethylamine (0.50 mL, 3.6 mmol) in PhMe (10 mL) was stirred at 90° C. The reaction was monitored by LCMS. At 18 h, the reaction mixture was cooled to rt and concentrated. The residue was dissolved in DCM (50 mL) and then washed with water (3×30 mL). The organics were passed through a hydrophobic frit, dried over Na₂SO₄, and concentrated. The crude material was purified via silica gel chromatography (20-60% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-((tert-butoxycarbonyl)amino)propoxy)butyl)(3-((tert-butoxycarbonyl)amino)propyl)carbamate (0.825 g, 1.01 mmol, 84.2%) as a tacky white foam. UPLC/ELSD: RT=3.39 min. MS (ES): m/z=839.2 [M+Na]⁺ for C₄₈H₈₅N₃O₇; ¹H NMR (300 MHz, CDCl₃): δ 5.34-5.43 (m, 1H), 5.30 (br. s, 1H), 4.73-5.00 (m, 1H), 4.41-4.59 (m, 1H), 3.46 (t, 2H, J=5.9 Hz), 3.41 (t, 2H, J=5.9 Hz), 3.00-3.36 (br. m, 8H), 2.20-2.43 (m, 2H), 0.93-2.09 (br. m, 34H), 1.43 (s, 18H), 1.02 (s, 3H), 0.91 (d, 3H, J=6.4 Hz), 0.86 (d, 6H, J=6.5 Hz), 0.67 (s, 3H).

### Step 8: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-aminopropoxy)butyl)(3-aminopropyl)carbamate dihydrochloride

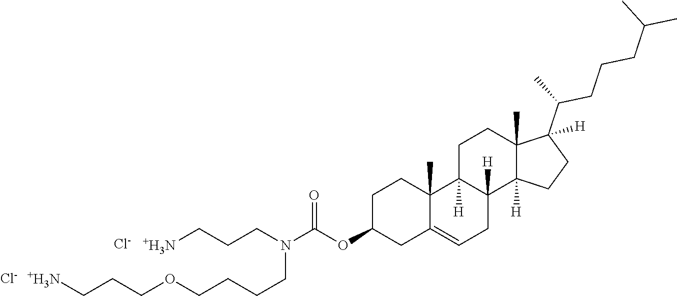

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-((tert-butoxycarbonyl)amino)propoxy)butyl)(3-((tert-butoxycarbonyl)amino)propyl)carbamate (0.809 g, 0.991 mmol) in iPrOH (6.0 mL) was added 5-6 N HCl in iPrOH (1.4 mL). The reaction mixture stirred at 40° C. and was monitored by LCMS. At 15.5 h, the reaction mixture was cooled to rt. ACN (18 mL) was added to the reaction mixture, and the suspension stirred at rt for 10 min. After this time, solids were collected by vacuum filtration and rinsed with 3:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-aminopropoxy)butyl)(3-aminopropyl)carbamate dihydrochloride (0.609 g, 0.828 mmol, 83.6%) as a white solid. UPLC/ELSD: RT=2.00 min. MS (ES): m/z=617.0 [M+H]⁺ for C₃₈H₆₉N₃O₃; ¹H NMR (300 MHz, CDCl₃): δ 8.51-8.82 (m, 3H), 8.05 (br. s, 3H), 5.33-5.42 (m, 1H), 4.42-4.57 (m, 1H), 3.63 (t, 2H, J=5.4 Hz), 2.97-3.58 (br. m, 10H), 2.19-2.43 (m, 2H), 0.93-2.13 (br. m, 34H), 1.02 (s, 3H), 0.91 (d, 3H, J=6.4 Hz), 0.86 (d, 3H, J=6.5 Hz), 0.86 (d, 3H, J=6.5 Hz), 0.67 (s, 3H).

### Y. Compound SA62: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-2-methyl-3-oxopropanoate trihydrochloride

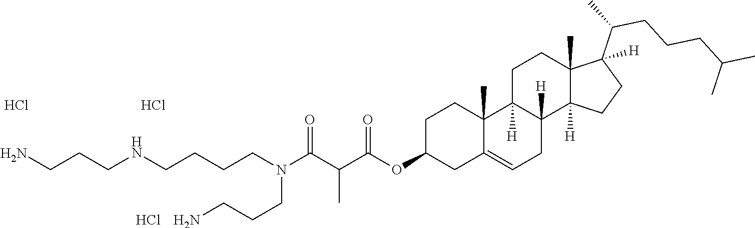

### Step 1: 1-(tert-Butyl) 3-((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 2-methylmalonate

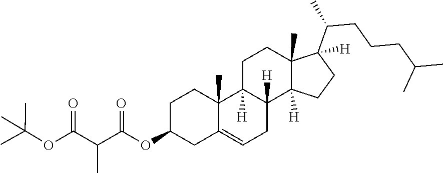

To a solution of cholesterol (1.85 g, 4.69 mmol) and 3-(tert-butoxy)-2-methyl-3-oxopropanoic acid (0.96 mL, 5.63 mmol) in dichloromethane (50 mL) stirring under nitrogen was added 1-ethyl-3-(3-dimethylaminopropyl)carbodiimide hydrochloride (1.36 g, 7.03 mmol). Then the reaction mixture was cooled to 0° C., and diisoproylethylamine (2.48 mL, 14.07 mmol) was added dropwise over 20 minutes. The resulting mixture was allowed to gradually warm to room temperature and proceed overnight. The mixture was then diluted with dichloromethane to 150 mL, washed with water (1×70 mL), saturated aqueous sodium bicarbonate (2×70 mL), and brine (1×70 mL), dried over sodium sulfate, filtered, and concentrated in vacuo to give a yellow oil. The oil was taken up in dichloromethane and purified on silica with a 0-25% ethyl acetate gradient in hexanes to give 1-(tert-butyl) 3-((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 2-methylmalonate as an oil (1.62 g, 2.99 mmol, 63.7%). UPLC/ELSD: RT: 3.41 min. MS (ES): m/z (MH⁺) 543.8 for C₃₅H₅₈O₄. ¹H NMR (300 MHz, CDCl₃) δ: ppm 5.31 (m, 1H), 4.58 (br. m, 1H), 3.21 (q, 1H, J=6 Hz), 2.27 (d, 2H, J=9 Hz), 1.87 (br. m, 6H), 1.50 (br. m, 6H), 1.39 (s, 12H), 1.28 (br. m, 12H), 1.07 (br. m, 8H), 0.95 (s, 4H), 0.91 (d, 2H, J=6 Hz), 0.86 (d, 4H, J=6 Hz), 0.80 (d, 8H, J=6 Hz), 0.61 (s, 3H).

### Step 2: 3-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-methyl-3-oxopropanoic acid

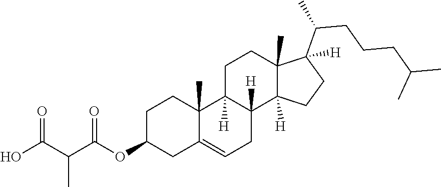

A solution of 1-(tert-butyl) 3-((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 2-methylmalonate (1.62 g, 2.99 mmol) in dichloromethane (50 mL) was cooled to 0° C. To this solution was added trifluoroacetic acid (3.43 mL, 44.79 mmol) dropwise over 20 minutes. The reaction mixture was allowed to gradually warm to room temperature and proceed for 5 hours, slowly turning a light pink. The crude reaction mixture was concentrated in vacuo to a pink solid, taken up in DCM, and purified on silica with a 0-40% ethyl acetate gradient in hexanes to give 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-methyl-3-oxopropanoic acid as a white solid (1.05 g, 2.16 mmol, 72.2%). UPLC/ELSD: RT: 3.02 min. MS (ES): m/z (MH⁺) 487.7 for C₃₁H₅₀O₄. ¹H NMR (300 MHz, CDCl₃) δ: ppm 11.03 (br. s, 1H), 5.40 (br. d, 1H), 4.72 (br. m, 1H), 3.49 (q, 1H, J=6 Hz), 2.38 (d, 2H, J=9 Hz), 2.01 (br. m, 5H), 1.61 (br. m, 5H), 1.50 (d, 5H, J=6 Hz), 1.27 (br. m, 12H), 1.04 (s, 5H), 0.95 (d, 4H, J=6 Hz), 0.90 (d, 6H, J=6 Hz), 0.70 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,16-trimethyl-4,15-dioxo-3-oxa-5,9,14-triazaheptadecan-17-oate

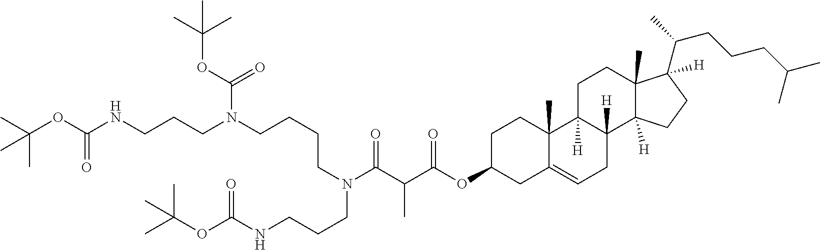

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-methyl-3-oxopropanoic acid (0.50 g, 1.02 mmol) in dichloromethane (10 mL) stirring under nitrogen was added tert-butyl (3-((tert-butoxycarbonyl)amino)propyl)(4-((3-((tert-butoxycarbonyl)amino)propyl)amino)butyl)carbamate (0.72 g, 1.42 mmol), dimethylaminopyridine (0.01 g, 0.10 mmol), and 1-ethyl-3-(3-dimethylaminopropyl)carbodiimide hydrochloride (0.30 g, 1.53 mmol). The resulting solution was cooled to 0° C. and diisopropylethylamine (0.54 mL, 3.05 mmol) was added dropwise. The mixture was allowed to gradually warm to room temperature and proceed overnight. Then, the solution was diluted with dichloromethane, washed with water (3×10 mL), dried over sodium sulfate, filtered, and concentrated to give an oil. The oil was taken up in DCM and purified on silica with a 0-60% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,16-trimethyl-4,15-dioxo-3-oxa-5,9,14-triazaheptadecan-17-oate as a light yellow oil (0.18 g, 0.19 mmol, 18.2%). UPLC/ELSD: RT: 3.26 min. MS (ES): m/z (MH⁺) 972.4 for C₅₆H₉₈N₄O₉. ¹H NMR (300 MHz, CDCl₃) δ: ppm 5.36 (br. s, 1H), 4.58 (br. m, 1H), 4.10 (q, 1H, J=6 Hz), 3.36 (br. m, 13H), 2.26 (br. m, 3H), 2.01 (s, 4H), 1.80 (br. m, 10H), 1.43 (br. m, 47H), 1.23 (t, 4H, J=9 Hz), 1.08 (br. m, 7H), 0.97 (s, 8H), 0.90 (d, 4H, J=9 Hz), 0.84 (d, 6H, J=6 Hz), 0.65 (s, 3H).

### Step 4: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-2-methyl-3-oxopropanoate trihydrochloride

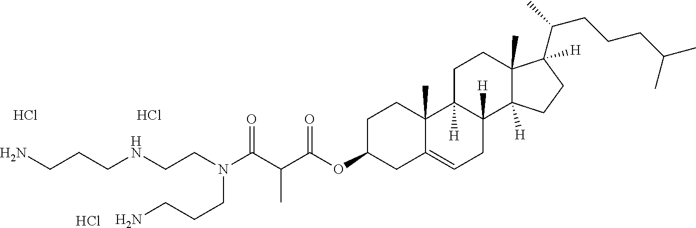

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,16-trimethyl-4,15-dioxo-3-oxa-5,9,14-triazaheptadecan-17-oate (0.18 g, 0.19 mmol) in isopropanol (10 mL) stirring under nitrogen was added hydrochloric acid (5.5 M in isopropanol, 0.37 mL, 1.85 mmol) dropwise. The mixture was heated to 45° C. and allowed to stir overnight. Then, the solution was cooled to room temperature and acetonitrile (5 mL) was added to the mixture. It was then sonicated to remove precipitated solid off the side of the flask. After stirring for 30 minutes after sonication, the solid was filtered out by vacuum filtration, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-2-methyl-3-oxopropanoate trihydrochloride as a white solid (0.06 g, 0.07 mmol, 38.7%). UPLC/ELSD: RT: 1.70 min. MS (ES): m/z (MH⁺) 672.1 for C₄₁H₇₇Cl₃N₄O₃. ¹H NMR (300 MHz, CD₃OD) δ: ppm 5.41 (s, 1H), 4.88 (br. m, 10H), 4.58 (br. m, 1H), 3.92 (br. m, 1H), 3.56 (br. m, 4H), 3.33 (s, 3H), 3.10 (br. m, 8H), 2.34 (br. m, 2H), 2.05 (br. m, 15H), 1.54 (br. m, 8H), 1.38 (br. m, 8H), 1.17 (d, 9H, J=6 Hz), 1.06 (s, 6H), 0.97 (d, 4H, J=6 Hz), 0.90 (d, 6H, J=6 Hz), 0.73 (s, 3H).

### Z. Compound SA63: (3S,8S,9S,10R,13S,14S,17S)-17-(2-Hydroxy-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate

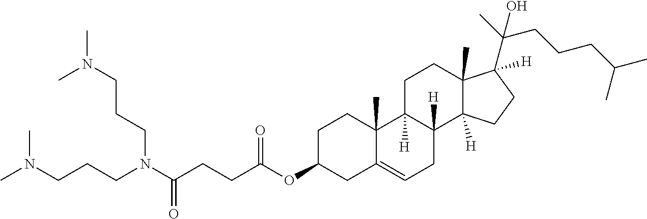

### Step 1: (3S,8S,9S,10R,13S,14S,17S)-17-(2-Hydroxy-6-methylheptan-2-yl)-10,13-dimethyl-2,3,4, 7, 8,9,10,11,12,13,14,15,16,17-tetradecahydro-1H-cyclopenta[a]phenanthren-3-ol

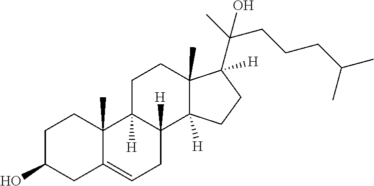

A mixture of magnesium turnings (2.21 g, 90.87 mmol) and iodine (1.54 g, 6.06 mmol) were purged twice with vacuum and nitrogen and then held under nitrogen. To this mixture was added dry tetrahydrofuran (50 ml) and set stirring under nitrogen. To this mixture was added 1-bromo-4-methylpentane (8.82 mL, 60.58 mmol) dropwise over 10 minutes, and then the reaction was allowed to proceed for one hour at room temperature. Following, the reaction mixture was refluxed at 66° C. for 3 hours, during which the grey reaction slurry turned to a clear colorless solution with some undissolved magnesium. The reaction was then cooled to 0° C. upon which the solution became cloudy again. At 0° C., a solution of pregnenolone (5.75 g, 18.17 mmol) in dry tetrahydrofuran (25 mL) was added dropwise over an hour, during which the reaction mixture solidified. Following, the solution was warmed to room temperature, an additional 50 mL tetrahydrofuran was added, and the reaction was allowed to continue at 30° C. overnight, during which the solidified mixture broke into smaller pieces stirring in the added solvent. The reaction was quenched the following day with saturated aqueous ammonium chloride (50 mL) and then diluted with 100 mL ethyl acetate. The aqueous layer was separated, and extracted again with 100 mL ethyl acetate. Then the organic layers were combined, washed with water (1×100 mL) and brine (1×100 mL), dried over sodium sulfate, filtered, and concentrated to dryness. The resulting residue was taken up in DCM and purified on silica with a 0-50% ethyl acetate gradient in hexanes to give (3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-ol as a white solid (2.68 g, 6.64 mmol, 36.6%). UPLC/ELSD: RT: 2.13 min. MS (ES): m/z (MH⁺) 403.7 for C₂₇H₄₆O₂. ¹H NMR (300 MHz, CDCl₃) δ: ppm 5.36 (br. d, 1H, J=6 Hz), 3.54 (br. m, 1H), 2.29 (br. m, 2H), 2.06 (br. m, 2H), 1.85 (br. m, 16H), 1.29 (s, 6H), 1.17 (br. m, 6H), 1.03 (s, 6H), 0.88 (d, 10H, J=6 Hz).

### Step 2: 4-(((3S,8S,9S,10R,13S,14S,17S)-17-(2-Hydroxy-6-methylheptan-2-yl)-10,13-dimethyl-2,3,4,7,8,9,10,11,12,13,14,15,16,17-tetradecahydro-1H-cyclopenta[a]phenanthren-3-yl)oxy)butanoic acid

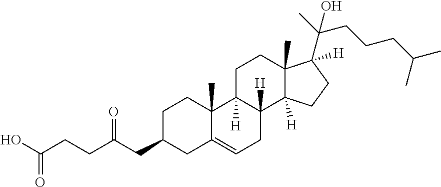

To a solution of (3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-ol (0.50 g, 1.24 mmol) in dichloromethane (10 mL) stirring under nitrogen was added succinic anhydride (0.12 g, 1.24 mmol). Then pyridine (0.17 mL, 1.24 mmol) was added dropwise at room temperature, and the mixture was refluxed at 40° C. overnight upon which all solid went into solution. The following day, TLC revealed incomplete conversion, and dimethylaminopyridine (0.05 g, 0.41 mmol) and succinic anhydride (0.03 g, 0.25 mmol) were added before the reaction mixture was allowed to reflux overnight again at 40° C. The following morning, the mixture was concentrated in vacuo to a yellow oil. The yellow oil was taken up in dichloromethane and purified on silica with a 0-30% ethyl acetate gradient in hexanes to give 4-(((3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-yl)oxy)butanoic acid as a white solid (0.27 g, 0.54 mmol, 43.3%). UPLC/ELSD: RT: 2.20 min. MS (ES): m/z (MH⁺) 503.8 for C₃₁H₅₀O₅. ¹H NMR (300 MHz, CDCl₃) δ: ppm 6.60 (br. s, 1H), 5.38 (br. s, 1H), 4.64 (br. m, 1H), 4.13 (q, 1H, J=6 Hz), 2.66 (dd, 4H, J=6 Hz), 2.33 (d, 2H, J=6 Hz), 2.05 (br. m, 2H), 1.84 (br. m, 3H), 1.51 (br. m, 12H), 1.28 (br. m, 8H), 1.13 (br. m, 5H), 1.02 (s, 4H), 0.86 (d, 10H, J=6 Hz).

### Step 3: (3S,8S,9S,10R,13S,14S,17S)-17-(2-Hydroxy-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate

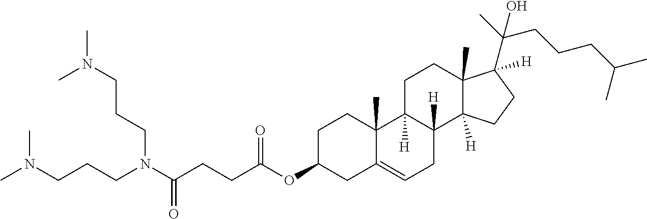

To a solution of 4-(((3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-yl)oxy)butanoic acid (0.35 g, 0.68 mmol) in dichloromethane (10 mL) stirring under nitrogen was added tetramethyldipropylenetriamine (0.24 mL, 1.02 mmol), dimethylaminopyridine (0.01 g, 0.07 mmol), and 1-ethyl-3-(3-dimethylaminopropyl)carbodiimide hydrochloride (0.20 g, 1.02 mmol). The resulting solution was cooled to 0° C., and diisopropylethylamine (0.36 mL, 2.04 mmol) was added dropwise. The mixture was allowed to gradually warm to room temperature and proceed overnight. Then, the solution was diluted with dichloromethane, washed with saturated aqueous sodium bicarbonate (1×10 mL) and brine (1×10 mL), dried over sodium sulfate, filtered, and concentrated to give an oil. The oil was taken up in DCM and purified on silica with a 0-60% (80:19:1 DCM/MeOH/NH₄OH) gradient in DCM to give (3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate as a light yellow oil (0.07 g, 0.09 mmol, 13.6%). UPLC/ELSD: RT: 1.25 min. MS (ES): m/z (MH⁺) 673.0 for C₄₁H₇₃N₃O₄. ¹H NMR (300 MHz, CDCl₃) δ: ppm 5.37 (br. s, 1H), 4.64 (br. m, 1H), 3.35 (br. t, 4H, J=9 Hz), 2.64 (s, 4H), 2.28 (br. m, 6H), 2.22 (s, 12H), 1.83 (br. m, 4H), 1.60 (br. m, 15H), 1.28 (br. s, 7H), 1.13 (br. m, 5H), 1.02 (s, 4H), 0.89 (d, 9H, J=6 Hz).

### AA. Compound SA64: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-(dimethylamino)octyl)(3-(dimethylamino)propyl)carbamate

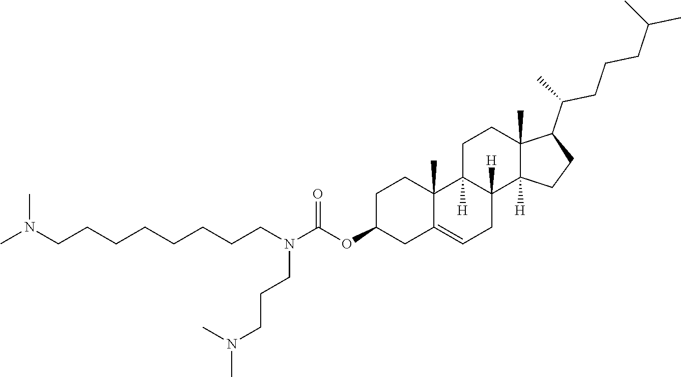

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)(3-aminopropyl)carbamate (105 mg, 0.15 mmol) and sodium acetate trihydrate (208 mg, 1.53 mmol) in 6 mL methanol at room temperature was added formaldehyde (0.12 mL, 37 wt % in water, 1.53 mmol) and sodium cyanoborohydride (96.1 mg, 1.53 mmol). The solution was stirred at room temperature for 16 hours, after which no starting aminosterol remained by LCMS. The mixture was diluted with 2 M aqueous NaOH solution and extracted three times with DCM. The organics were combined, washed once with brine, dried (MgSO₄), filtered, and concentrated. The residue was purified by silica gel chromatography (0-50% (mixture of 1% concentrated aq. NH₄OH and 20% MeOH in DCM) in DCM) to give 3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-(dimethylamino)octyl)(3-(dimethylamino)propyl)carbamate (63.2 mg, 0.091 mmol, 60%) as a colorless oil. UPLC/ELSD: RT=2.14 min. MS (ES): m/z (MH⁺) 671.2 for C₄₃H₈₀N₃O₂. ¹H NMR (300 MHz, CDCl₃) δ: ppm 5.35 (d, 1H, J=5 Hz); 4.48 (septet, 1H, J=5 Hz); 3.19 (s, 4H); 2.39-2.27 (m, 12H); 2.25 (s, 6H); 2.22 (s, 6H); 2.03-1.63 (m, 8H); 1.58-1.04 (m, 23H); 1.00 (s, 6H); 0.90 (d, 3H, J=6 Hz); 0.86 (d, 3H, J=1 Hz); 0.84 (d, 3H, J=1 Hz); 0.66 (s, 3H).

### AB. Compound SA65: ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride

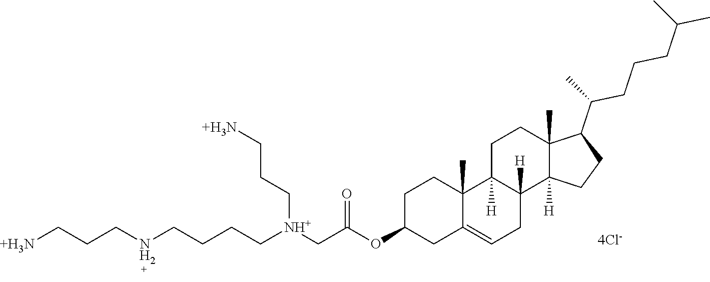

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloroacetate

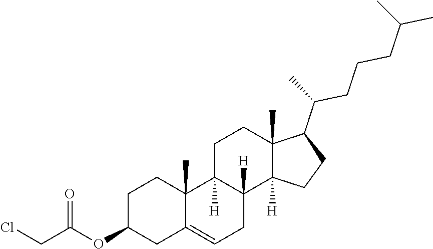

Cholesterol (2 g, 5.17 mmol), chloroacetic acid (573 mg, 5.69 mmol), DMAP (63 mg, 0.52 mmol), and DCC (1.17 g, 5.69 mmol) were dissolved in 10 mL DCM. The solution was stirred at room temperature for 17 hours. The mixture was filtered, and the filtrate was washed with ethyl acetate. The filtered solution was concentrated and dissolved in ethyl acetate. The organic layer was washed once with water and brine, dried (MgSO₄), filtered, and concentrated. The residue was purified by silica gel chromatography (0-40% ethyl acetate in hexanes) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloroacetate (0.71 g, 1.53 mmol, 30%) as a white solid. UPLC/ELSD: RT=3.43 min. ¹H NMR (300 MHz, CDCl₃) δ: ppm 5.40 (d, 1H, J=5 Hz); 4.48 (septet, 1H, J=4 Hz); 4.03 (s, 2H); 2.36 (d, 2H, J=8 Hz); 2.06-1.77 (m, 5H); 1.64-1.05 (m, 21H); 1.02 (s, 3H); 0.91 (d, 3H, J=6 Hz); 0.88 (s, 3H); 0.86 (s, 3H); 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2, 2-dimethyl-4-oxo-3-oxa-5,9,14-triazahexadecan-16-oate

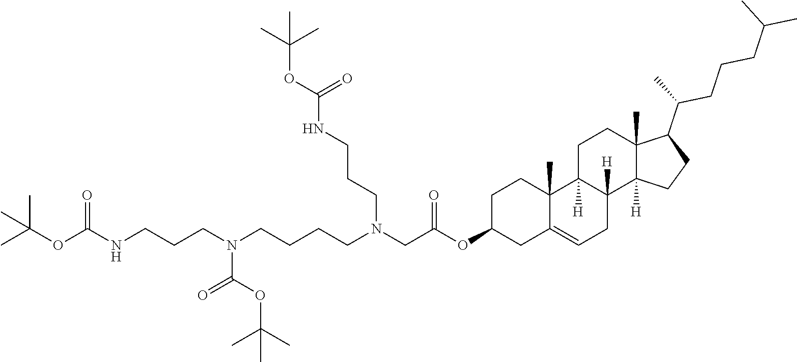

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloroacetate (350 mg, 0.75 mmol) and NaI (113 mg, 0.75 mmol) in 7.5 mL acetonitrile at room temperature was added a solution of tert-butyl N-{3-[(tert-butoxycarbonyl)amino]propyl}-N-[4-({3-[(tert-butoxycarbonyl)amino]propyl}amino)butyl]carbamate (378 mg, 0.75 mmol) and N,N-diisopropylethylamine (0.2 mL, 1.13 mmol) in 7.5 mL acetonitrile. The solution was stirred at 60° C. for 18 hours. The mixture was diluted with ethyl acetate, washed once with water and brine, dried over MgSO₄, filtered, and concentrated. The residue was purified by silica gel chromatography (0-100% (mixture of 1% NH₄OH, 20% MeOH in DCM) in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate (590 mg, 0.63 mmol, 84%) as a colorless oil. UPLC/ELSD: RT=2.94 min. MS (ES): m/z (MH⁺) 930.0 for C₅₄H₉₇N₄O₈. ¹H NMR (300 MHz, CDCl₃) δ: ppm 5.36 (d, 1H, J=5 Hz); 5.27 (br s, 1H); 4.80 (br s, 1H); 4.74-4.57 (m, 1H); 3.40 (br s, 1H); 3.31-2.99 (m, 7H); 2.75 (br s, 3H); 2.31 (d, 2H, J=8 Hz); 2.06-1.03 (m, 64H); 1.00 (s, 3H); 0.91 (d, 3H, J=6 Hz); 0.87 (s, 3H); 0.85 (s, 3H); 0.67 (s, 3H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride

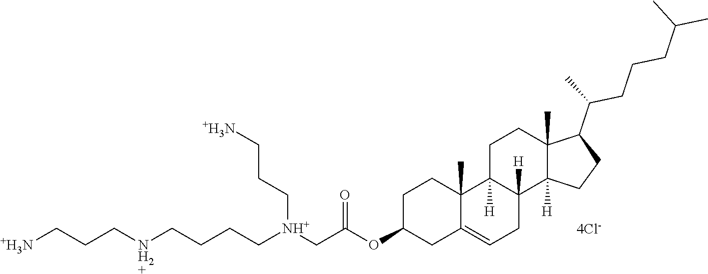

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate (590 mg, 0.64 mmol) in isopropanol (15 mL) was added a 5 M HCl solution in isopropanol (15 mL, 6.4 mmol). The solution was stirred at 40° C. for 41 hours. The mixture was cooled to room temperature and diluted with acetonitrile (15 mL). Resulting solid was precipitated by centrifugation (5000 g, 5 min). The supernatant was removed, and the pellet was dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate (350 mg, 0.45 mmol, 71%) as a white powder. UPLC/ELSD: RT=1.83 min. MS (ES): m/z ([M−3HCl—Cl⁻]⁺) 629.6 for C₃₉H₇₃N₄O₂. ¹H NMR (300 MHz, CD₃OD) δ: ppm 5.43 (d, 1H, J=4 Hz); 4.80-4.66 (m, 1H); 4.28 (s, 2H); 3.49-3.33 (m, 4H); 3.22-3.02 (m, 8H); 2.43 (d, 2H, J=7 Hz); 2.28-1.09 (m, 29H); 1.06 (s, 3H); 0.95 (d, 3H, J=6 Hz); 0.89 (s, 3H); 0.87 (s, 3H); 0.73 (s, 3H).

### AC. Compound SA66: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 2-((3-aminopropyl)(4-((3-aminopropyl)amino)butyl)amino)-2-oxoacetate trihydrochloride

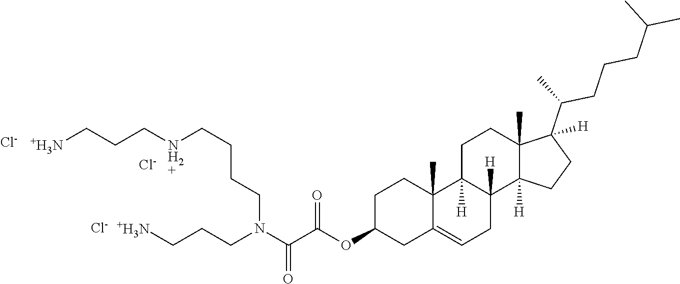

### Step 1: 2-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoacetic acid

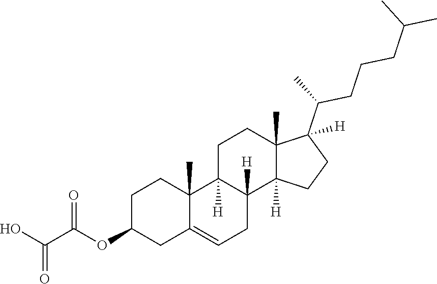

To a stirred solution of cholesterol (0.500 g, 1.29 mmol) in a mixture of Et₂O (6.5 mL) and DCM (2.0 mL) cooled to 0° C. in an ice bath was added oxalyl chloride (0.23 mL, 2.7 mmol) slowly dropwise. The reaction mixture was allowed to come to rt slowly and was monitored by TLC. At 24 h, the reaction mixture was cooled to 0° C. in an ice bath, and then water (3.0 mL) was added dropwise (CAUTION: VIGOROUS GAS EVOLUTION OCCURRED DURING ADDITION). The mixture stirred at rt for 1 h, and then the layers were separated. The aqueous layer was extracted with Et₂O (3×). The combined organics were washed with brine, dried over Na₂SO₄, and concentrated to afford 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoacetic acid (0.534 g, 1.16 mmol, 90.0%) as a white solid. UPLC/ELSD: RT=2.95 min. ¹H NMR (300 MHz, CDCl₃): δ 5.68 (br. s, 1H), 5.38-5.46 (m, 1H), 4.75-4.89 (m, 1H), 2.35-2.61 (m, 2H), 1.70-2.11 (br. m, 6H), 0.93-1.65 (br. m, 20H), 1.04 (s, 3H), 0.92 (d, 3H, J=6.5 Hz), 0.87 (d, 3H, J=6.6 Hz), 0.86 (d, 3H, J 5=6.5 Hz), 0.68 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 2-chloro-2-oxoacetate

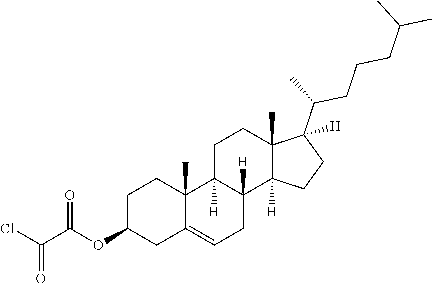

To a solution of 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoacetic acid (0.100 g, 0.218 mmol) and DMF (cat.) in DCM (2 mL) was added oxalyl chloride (0.03 mL, 0.4 mmol) slowly dropwise. The reaction mixture stirred at rt and was monitored by LCMS. At 40 min, the reaction mixture was concentrated, and then reconcentrated from PhMe to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloro-2-oxoacetate as a yellow solid. Material was carried forward without further purification assuming quantitative yield.

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazahexadecan-16-oate

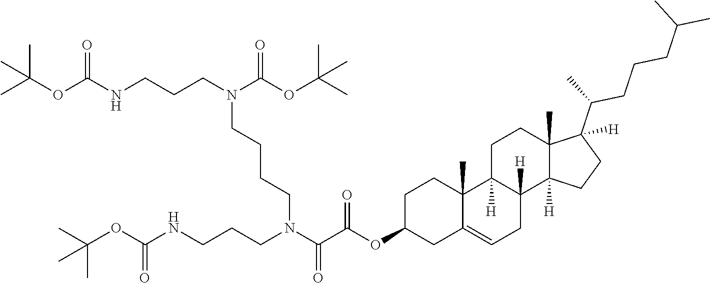

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloro-2-oxoacetate (0.104 g, 0.218 mmol) and triethylamine (0.10 mL, 0.71 mmol) in toluene (2.0 mL) cooled to 0° C. in an ice bath was added tert-butyl N-{3-[(tert-butoxycarbonyl)amino]propyl}-N-[4-({3-[(tert-butoxycarbonyl)amino]propyl}amino)butyl]carbamate (0.150 g, 0.298 mmol) in toluene (0.75 mL) dropwise. The reaction mixture stirred at rt and was monitored by LCMS. At 30 min, the reaction mixture stirred at 50° C. At 17 h, the reaction mixture was cooled to rt and then concentrated. The residue was taken up in DCM and washed with 5% aq. NaHCO₃ solution. The organics were passed through a hydrophobic frit, dried over Na₂SO₄, and concentrated. The crude material was purified via silica gel chromatography (0-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazahexadecan-16-oate (0.138 g, 0.146 mmol, 67.1%) as a yellow oil. UPLC/ELSD: RT=3.33 min. MS (ES): m/z=844.4 [(M+H)—(CH₃)₂C═CH₂—CO₂]⁺ for C₅₄H₉₄N₄O₉; ¹H NMR (300 MHz, CDCl₃): δ 5.38-5.45 (m, 1H), 5.21 (br. s, 1H), 4.65-4.87 (m, 2H), 3.32-3.47 (m, 2H), 3.02-3.31 (br. m, 10H), 2.35-2.53 (m, 2H), 0.94-2.08 (br. m, 34H), 1.46 (s, 9H), 1.44 (s, 18H), 1.02 (s, 3H), 0.92 (d, 3H, J=6.4 Hz), 0.87 (d, 3H, J=6.6 Hz), 0.86 (d, 3H, J=6.5 Hz), 0.68 (s, 3H).

### Step 4: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 2-((3-aminopropyl)(4-((3-aminopropyl)amino)butyl)amino)-2-oxoacetate trihydrochloride

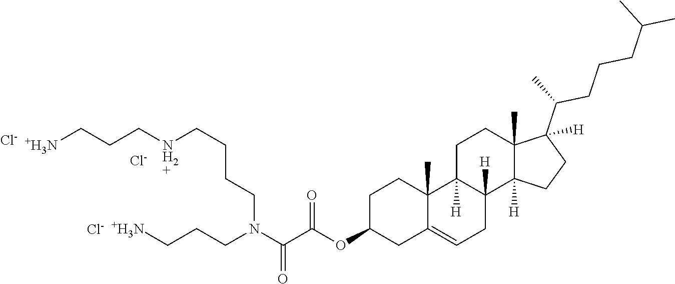

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazahexadecan-16-oate (0.132 g, 0.140 mmol) in iPrOH (1.3 mL) was added 5-6 N HCl in iPrOH (0.28 mL). The reaction mixture stirred at 40° C. and was monitored by LCMS. At 18 h, the reaction mixture was cooled to rt. ACN (3 mL) was added to the reaction mixture, and the suspension stirred at rt for 1 h. After this time, solids were collected by vacuum filtration to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((3-aminopropyl)(4-((3-aminopropyl)amino)butyl)amino)-2-oxoacetate trihydrochloride (0.085 g, 0.10 mmol, 73.2%) as a white solid. UPLC/ELSD: RT=1.70 min. MS (ES): m/z=643.8 [M+H]⁺ for C₃₉H₇₀N₄O₃; ¹H NMR (300 MHz, CD₃OD): δ 5.42-5.51 (m, 1H), 4.72-4.85 (m, 1H), 3.34-3.61 (br. m, 4H), 3.04-3.19 (br. m, 6H), 2.92-3.01 (m, 2H), 2.37-2.54 (m, 2H), 0.98-2.19 (br. m, 34H), 1.08 (s, 3H), 0.96 (d, 3H, J=6.4 Hz), 0.89 (d, 3H, J=6.6 Hz), 0.89 (d, 3H, J=6.6 Hz), 0.74 (s, 3H).

### AD. Compound SA67: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-(dimethylamino)propoxy)butyl)(3-(dimethylamino)propyl)carbamate

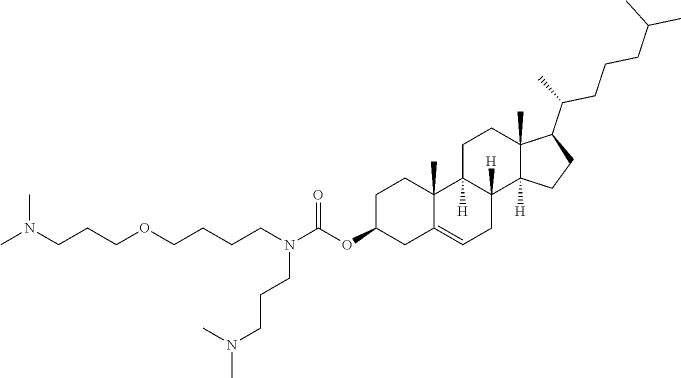

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-aminopropoxy)butyl)(3-aminopropyl)carbamate (100 mg, 0.15 mmol) and sodium acetate trihydrate (197.5 mg, 1.45 mmol) in 5.8 mL methanol at room temperature was added formaldehyde (0.11 mL, 37 wt % in water, 1.45 mmol) and sodium cyanoborohydride (91.2 mg, 1.45 mmol). The solution was stirred at room temperature for 6 hours, after which no starting aminosterol remained by LCMS. The mixture was diluted with 2 M aqueous NaOH solution and extracted three times with DCM. The organics were combined, washed once with brine, dried (MgSO₄), filtered, and concentrated. The residue was purified by silica gel chromatography (0-20% (mixture of 1% NH₄OH and 20% MeOH in DCM) in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-(dimethylamino)propoxy)butyl)(3-(dimethylamino)propyl)carbamate (35.6 mg, 0.051 mmol, 35%) as a colorless oil. UPLC/ELSD: RT=2.01 min. MS (ES): m/z (MH⁺) 673.0 for C₄₂H₇₈N₃O₃. ¹H NMR (300 MHz, CDCl₃) δ: ppm 5.36 (d, 1H, J=5 Hz); 4.49 (septet, 1H, J=5 Hz); 3.48-3.37 (m, 4H); 3.23 (s, 4H); 2.48-2.30 (m, 12H); 2.28 (s, 6H); 2.24 (s, 6H); 2.04-1.66 (m, 10H); 1.64-1.04 (m, 15H); 1.01 (s, 6H); 0.91 (d, 3H, J=6 Hz); 0.87 (d, 3H, J=1 Hz); 0.85 (d, 3H, J=1 Hz); 0.67 (s, 3H).

### AE. Compound SA68: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-(dimethylamino)propyl)(4-((3-(dimethylamino)propyl)(methyl)amino)butyl)carbamate

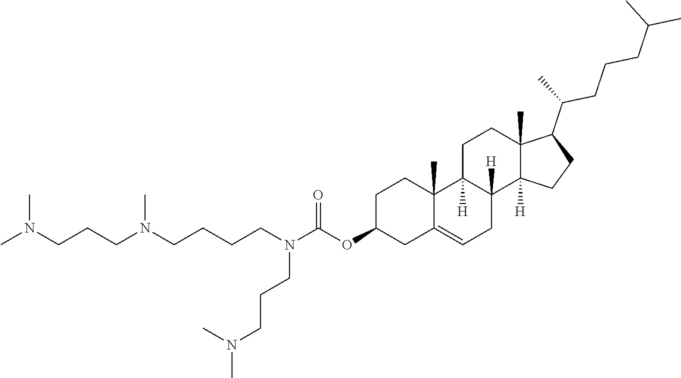

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate trihydrochloride (144 mg, 0.2 mmol) and sodium acetate trihydrate (162.3 mg, 1.19 mmol) in 2 mL methanol at room temperature was added formaldehyde (0.094 mL, 37 wt % in water, 1.19 mmol) and sodium cyanoborohydride (75 mg, 1.19 mmol). The solution was stirred at room temperature for 17 hours, after which no starting aminosterol remained by LCMS. The mixture was diluted with 2 M aqueous NaOH solution and extracted three times with DCM. The organics were combined, washed once with brine, dried (MgSO₄), filtered and concentrated. The residue was purified by silica gel chromatography (0-20% (mixture of 2% concentrated aq. NH₄OH and 20% MeOH in DCM) in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-(dimethylamino)propyl)(4-((3-(dimethylamino)propyl)(methyl)amino)butyl)carbamate (31.7 mg, 0.044 mmol, 22%) as a colorless oil. UPLC/ELSD: RT=1.71 min. MS (ES): m/z (MH⁺) 685.6 for C₄₃H₈₁N₄O₂. ¹H NMR (300 MHz, CDCl₃) δ: ppm 5.36 (d, 1H, J=5 Hz); 4.49 (septet, 1H, J=5 Hz); 3.47 (s, 4H); 3.22 (s, 4H); 2.38-2.24 (m, 12H); 2.22 (s, 6H); 2.21 (s, 6H); 2.20 (s, 3H); 2.05-1.95 (m, 2H); 1.72-1.06 (m, 23H); 1.01 (s, 6H); 0.91 (d, 3H, J=6 Hz); 0.87 (d, 3H, J=1 Hz); 0.85 (d, 3H, J=1 Hz); 0.67 (s, 3H).

### AF. Compound SA69: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride

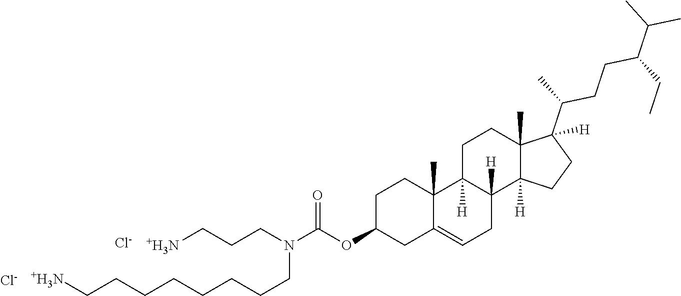

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate

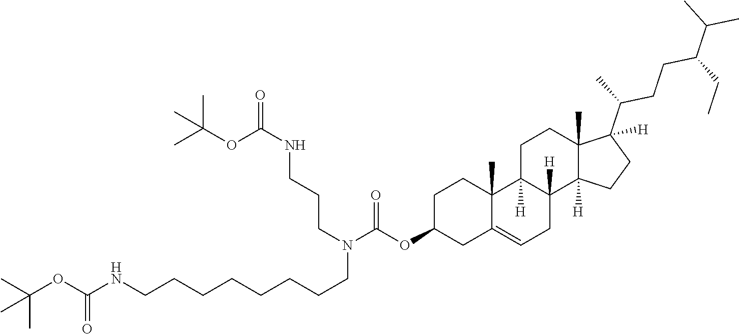

β-Sitosterol 4-nitrophenyl carbonate (0.300 g, 0.517 mmol), tert-butyl N-[3-({8-[(tert-butoxycarbonyl)amino]octyl}amino)propyl]carbamate (0.260 g, 0.647 mmol), and triethylamine (0.22 mL, 1.6 mmol) were combined in PhMe (4.5 mL). The reaction mixture stirred at 90° C. and was monitored by LCMS. At 18.25 h, the reaction mixture was cooled to rt and concentrated. The residue was taken up in DCM (20 mL) and washed with water (3×). The organic layer was passed through a hydrophobic frit, dried over Na₂SO₄, and concentrated. The crude material was purified via silica gel chromatography (20-50% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate (0.327 g, 0.388 mmol, 75.0%) as a white foam. UPLC/ELSD: RT=3.74 min. MS (ES): m/z=842.9 [M+H]⁺ for C₅₁H₉₁N₃O₆; ¹H NMR (300 MHz, CDCl₃): δ 5.15-5.47 (m, 2H), 4.40-4.86 (m, 2H), 2.98-3.41 (br. m, 8H). 2.20-2.45 (m, 2H), 1.76-2.12 (br. m, 5H), 0.89-1.75 (br. m, 54H), 1.02 (s, 3H), 0.92 (d, 3H, J=6.4 Hz), 0.77-0.88 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl(8-aminooctyl)(3-aminopropyl)carbamate dihydrochloride

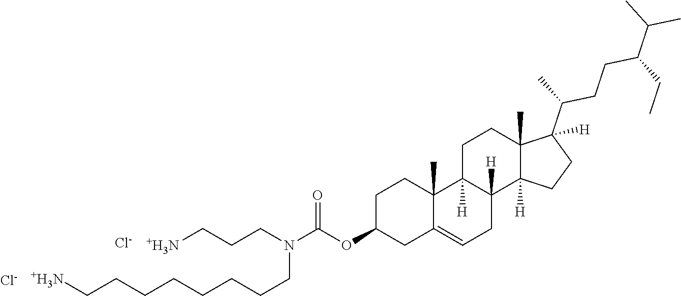

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate (0.315 g, 0.374 mmol) in iPrOH (4.0 mL) was added 5-6 N HCl in iPrOH (0.53 mL). The reaction mixture stirred at 40° C. and was monitored by LCMS. At 18 h, the reaction mixture was cooled to rt and ACN (12 mL) was added. The solids were collected via vacuum filtration and rinsed with 3:1 ACN/iPrOH to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride (0.236 g, 0.309 mmol, 82.5%) as a white solid. UPLC/ELSD: RT=3.54 min. MS (ES): m/z=342.6 [M+2Na]²⁺ for C₄₁H₇₇Cl₂N₃O₂; ¹H NMR (300 MHz, CDCl₃): δ 8.33 (br. s, 3H), 8.22 (br. s, 3H), 5.31-5.42 (m, 1H), 4.38-4.53 (m, 1H), 2.92-3.53 (br. m, 8H), 2.20-2.42 (m, 2H), 1.72-2.17 (br. m, 10H), 0.94-1.71 (br. m, 31H), 1.02 (s, 3H), 0.92 (d, 3H, J=6.3 Hz), 0.77-0.89 (m, 9H), 0.68 (s, 3H).

### AG. Compound SA70

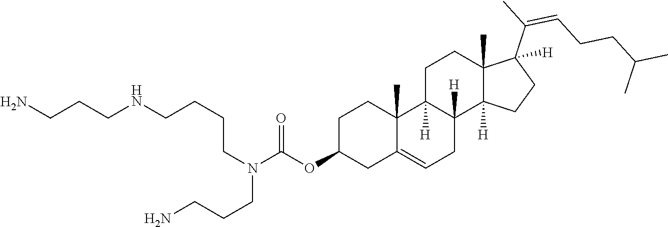

SA70 ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate analog olefin elimination by-product from hydroxycholesterol)

### AH. Compound SA71: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(3-aminopropyl)glycinate trihydrochloride

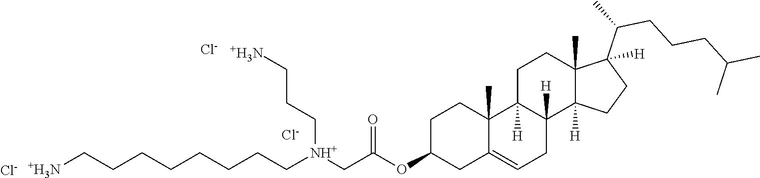

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(3-((tert-butoxycarbonyl)amino)propyl)glycinate

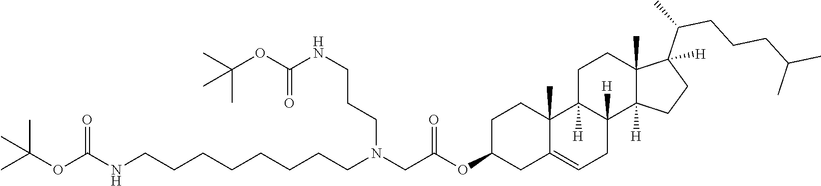

Cholesteryl chloroacetate (0.227 g, 0.490 mmol), tert-butyl N-[3-({8-[(tert-butoxycarbonyl)amino]octyl}amino)propyl]carbamate (0.236 g, 0.589 mmol), potassium carbonate (0.136 g, 0.980 mmol), and potassium iodide (0.008 g, 0.05 mmol) were combined in THF (3.5 mL). The reaction mixture stirred at 65° C. and was monitored by LCMS. At 4 h, the reaction mixture stirred at 60° C. At 93 h, the reaction mixture was cooled to rt. The reaction mixture was concentrated and then taken up in DCM. The organics was washed with water, passed through a hydrophobic frit, dried over Na₂SO₄, and concentrated. The crude material was purified via silica gel chromatography (20-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(3-((tert-butoxycarbonyl)amino)propyl)glycinate (0.318 g, 0.384 mmol, 78.3%) as a clear oil. UPLC/ELSD: RT=3.62 min. MS (ES): m/z=829.0 [M+H]⁺ for C₅₀Hs₉N₃O₆; ¹H NMR (300 MHz, CDCl₃): δ 5.45 (br. s, 1H), 5.35-5.41 (m, 1H), 4.58-4.72 (m, 1H), 4.50 (br. s, 1H), 3.25 (s, 2H), 3.20 (dt, 2H, J=5.7, 6.0 Hz), 3.09 (dt, 2H, J=6.4, 5.8 Hz), 2.59 (t, 2H, J=6.4 Hz), 2.50 (t, 2H, J=7.5 Hz), 2.28-2.36 (m, 2H), 1.75-2.08 (br. m, 5H), 0.94-1.70 (br. m, 53H), 1.02 (s, 3H), 0.91 (d, 3H, J=6.5 Hz), 0.87 (d, 3H, J=6.5 Hz), 0.86 (d, 3H, J=6.6 Hz), 0.68 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(3-aminopropyl)glycinate trihydrochloride

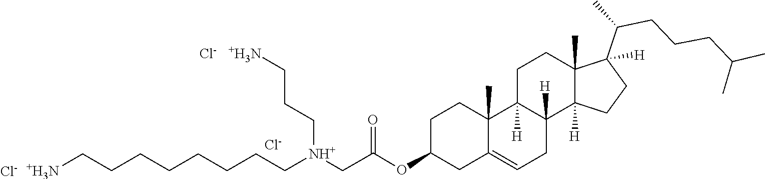

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(3-((tert-butoxycarbonyl)amino)propyl)glycinate (0.310 g, 0.374 mmol) in iPrOH (4.0 mL) was added 5-6 N HCl in iPrOH (0.53 mL). The reaction mixture stirred at 40° C. and was monitored by LCMS. At 21.75 h, the reaction mixture was cooled to rt, and ACN (12 mL) was added. The solids were collected via vacuum filtration and rinsed with 3:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(3-aminopropyl)glycinate trihydrochloride (0.208 g, 0.248 mmol, 66.4%) as a white solid. UPLC/ELSD: RT=1.83 min. MS (ES): m/z=335.4 [M+2Na]²′ for C₄₀H₇₃N₃O₂; ¹H NMR (300 MHz, CDCl₃): δ 10.72 (br. s, 1H), 8.41 (br. s, 3H), 8.27 (br. s, 3H), 5.38-5.48 (m, 1H), 4.59-4.82 (m, 1H), 2.91-4.42 (br. m, 10H), 2.22-2.72 (br. m, 4H), 1.72-2.18 (br. m, 10H), 0.93-1.70 (br. m, 28H), 1.01 (s, 3H), 0.91 (d, 3H, J=5.5 Hz), 0.86 (d, 6H, J=6.5 Hz), 0.67 (s, 3H).

### AI. Compound SA72: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-aminooctyl)(3-aminopropyl)amino)-4-oxobutanoate dihydrochloride

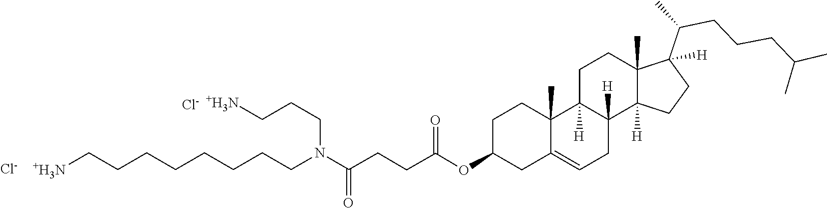

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-((tert-butoxycarbonyl)amino)octyl)(3-((tert-butoxycarbonyl)amino)propyl)amino)-4-oxobutanoate

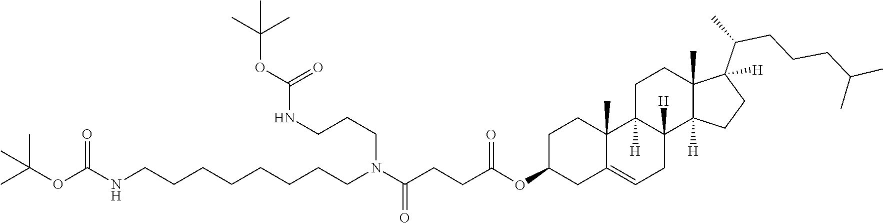

To a stirred solution of (−)-cholesterol NHS succinate (0.300 g, 0.514 mmol) in THF (3.0 mL) was added tert-butyl N-[3-({8-[(tert-butoxycarbonyl)amino]octyl}amino)propyl]carbamate (0.258 g, 0.642 mmol) in THF (1.0 mL). The reaction mixture stirred at rt and was monitored by LCMS. At 19 h, the reaction mixture was stirred at 50° C. At 23 h, the reaction mixture was cooled to rt and then concentrated. The residue was taken up in DCM and washed with water. The organics were passed through a hydrophobic frit, dried over Na₂SO₄, and concentrated. The crude material was purified via silica gel chromatography (20-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-((tert-butoxycarbonyl)amino)octyl)(3-((tert-butoxycarbonyl)amino)propyl)amino)-4-oxobutanoate (0.315 g, 0.362 mmol, 70.4%) as a white foam. UPLC/ELSD: RT=3.96 min. MS (ES): m/z=871.0 [M+H]⁺ for C₅₂H₉₁N₃O₇; ¹H NMR (300 MHz, CDCl₃): δ 5.27-5.46 (m, 2H), 4.39-4.76 (m, 2H), 2.95-3.48 (br. m, 8H), 2.53-2.72 (m, 4H), 2.24-2.39 (m, 2H), 1.75-2.06 (br. m, 5H), 0.93-1.70 (br. m, 53H), 1.01 (s, 3H), 0.91 (d, 3H, J=6.4 Hz), 0.86 (d, 3H, J=6.5 Hz), 0.86 (d, 3H, J=6.6 Hz), 0.67 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-aminooctyl)(3-aminopropyl)amino)-4-oxobutanoate dihydrochloride

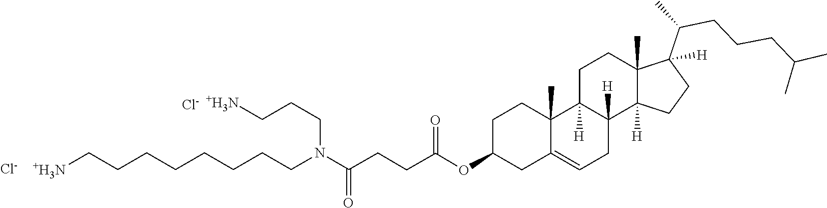

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-((tert-butoxycarbonyl)amino)octyl)(3-((tert-butoxycarbonyl)amino)propyl)amino)-4-oxobutanoate (0.307 g, 0.347 mmol) in iPrOH (4 mL) was added 5-6 N HCl in iPrOH (0.49 mL). The reaction mixture stirred at 40° C. and was monitored by LCMS. At 21.75 h, the reaction mixture was cooled to rt and then ACN (16 mL) was added. The solids were collected via vacuum filtration and rinsed with 4:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-aminooctyl)(3-aminopropyl)amino)-4-oxobutanoate dihydrochloride (0.169 g, 0.214 mmol, 61.8%) as a white solid. UPLC/ELSD: RT=2.15 min. MS (ES): m/z=336.0 [M+2H]²⁺ for C₄₂H₇₅N₃O₃; ¹H NMR (300 MHz, CDCl₃): δ 8.01-8.61 (m, 6H), 5.31-5.42 (m, 1H), 4.51-4.69 (m, 1H), 2.92-3.68 (br. m, 8H), 2.62 (s, 4H), 2.21-2.39 (m, 2H), 1.71-2.20 (br. m, 10H), 0.94-1.70 (br. m, 30H), 1.01 (s, 3H), 0.91 (d, 3H, J=6.4 Hz), 0.86 (d, 6H, J=6.5 Hz), 0.67 (s, 3H).

### AJ. Compound SA73: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate trihydrochloride

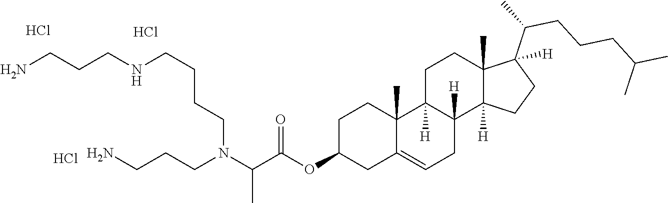

### Step 1: 9-(tert-Butoxycarbonyl)-14-(3-((tert-butoxycarbonyl)amino)propyl)-2,2,15-trimethyl-4-oxo-3-oxa-5,9,14-triazahexadecan-16-oic acid

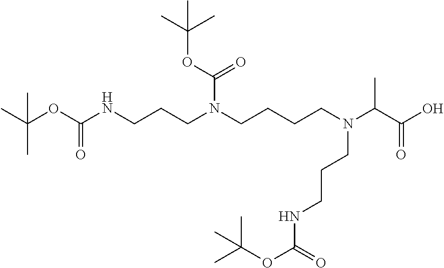 [SMILES: CC(C(=O)O)N(CCCCN(CCCNC(=O)OC(C)(C)C)C(=O)OC(C)(C)C)CCCNC(=O)OC(C)(C)C]

To a solution of tert-butyl (3-((tert-butoxycarbonyl)amino)propyl)(4-((3-((tert-butoxycarbonyl)amino)propyl)amino)butyl)carbamate (0.83 g, 1.65 mmol) and potassium hydroxide (0.37 g, 6.60 mmol) in methanol (10 mL) stirring under nitrogen was added 2-bromopropionic acid (0.30 mL, 3.30 mmol) dropwise at room temperature. The resulting solution was heated to 60° C. and allowed to proceed overnight. The following day, the solution was concentrated to an oil. The oil was taken up in dichloromethane and purified on silica with a 0-60% ethyl acetate gradient in hexanes to give 9-(tert-butoxycarbonyl)-14-(3-((tert-butoxycarbonyl)amino)propyl)-2,2,15-trimethyl-4-oxo-3-oxa-5,9,14-triazahexadecan-16-oic acid as an oil (0.13 g, 0.22 mmol, 13.2%). UPLC/ELSD: RT: 2.73 min. MS (ES): m/z (MH⁺) 575.8 for C₂₈H₅₄N₄O₈. ¹H NMR (300 MHz, CDCl₃) δ: ppm 7.31 (br. s, 1H), 5.72 (br. s, 1H), 3.17 (br. m, 13H), 1.90 (br. m, 2H), 1.64 (br. m, 7H), 1.40 (s, 26H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate

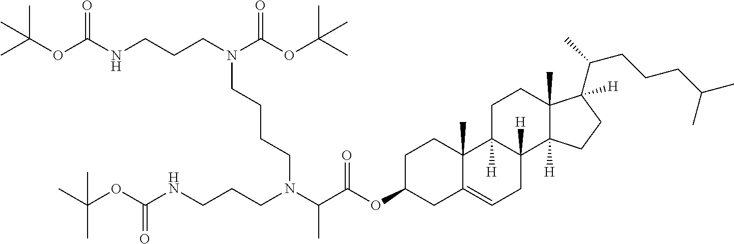

To a solution of cholesterol (0.10 g, 0.26 mmol) and 9-(tert-butoxycarbonyl)-14-(3-((tert-butoxycarbonyl)amino)propyl)-2,2,15-trimethyl-4-oxo-3-oxa-5,9,14-triazahexadecan-16-oic acid (0.13 g, 0.22 mmol) in dichloromethane (10 mL) stirring under nitrogen was added dimethylaminopyridine (0.01 g, 0.04 mmol) and 1-ethyl-3-(3-dimethylaminopropyl)carbodiimide hydrochloride (0.06 g, 0.33 mmol).

The resulting solution was cooled to 0° C. and diisopropylethylamine (0.12 mL, 0.65 mmol) was added dropwise. The mixture was allowed to gradually warm to room temperature and proceed overnight. Then, the solution was diluted with dichloromethane, washed with water (1×10 mL), saturated aqueous sodium bicarbonate (1×10 mL) and brine (1×10 mL), dried over sodium sulfate, filtered, and concentrated to an oil. The oil was taken up in DCM and purified on silica with a 0-25% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate as a colorless oil (0.05 g, 0.05 mmol, 21.9%). UPLC/ELSD: RT: 2.83 min. MS (ES): m/z (MH⁺) 944.4 for C₅₅H₉₈N₄O₈. The compound was not analyzed by H-NMR so as not to lose precious material needed for the following reaction.

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate trihydrochloride

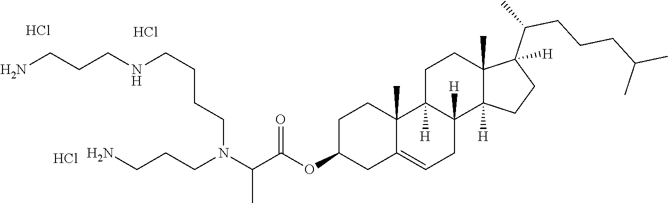

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate (0.05 g, 0.05 mmol) in 2-propanol (5 mL) stirring under nitrogen was added hydrochloric acid (5.5M in 2-propanol, 0.10 mL, 0.48 mmol) dropwise. The mixture was heated to 45° C. and allowed to stir overnight. Then, the solution was cooled to room temperature, and acetonitrile (3 mL) was added to the mixture. It was then sonicated to remove precipitated solid off the side of the flask. After stirring for 30 minutes after sonication, the solid was filtered out by vacuum filtration, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate trihydrochloride as a white solid (0.03 g, 0.03 mmol, 62.8%). UPLC/ELSD: RT: 1.51 min. MS (ES): m/z (MH⁺) 644.1 for C₄₀H₇₇Cl₃N₄O₂. ¹H NMR (300 MHz, CD₃OD) S: ppm 5.54 (br. s, 1H), 4.50 (br. m, 1H), 3.33 (br. d, 8H), 3.12 (br. m, 9H), 2.45 (br. m, 2H), 2.00 (br. m, 15H), 1.55 (br. m, 17H), 1.19 (br. m, 14H), 0.96 (d, 4H, J=6 Hz), 0.90 (d, 7H, J=6 Hz), 0.74 (s, 3H).

### AK. Compound SA74: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride

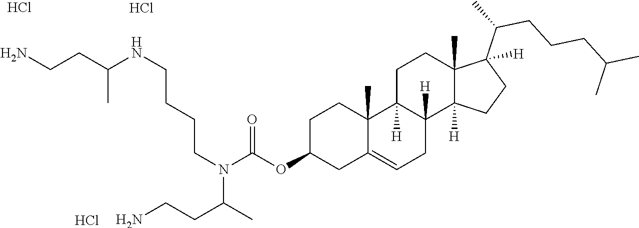

### Step 1: tert-butyl (3-((4-nitrophenyl)sulfonamido)butyl)carbamate

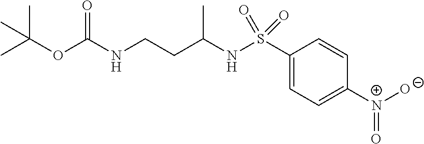 [SMILES: CC(CCNC(=O)OC(C)(C)C)NS(=O)(=O)c1ccc([N+](=O)[O-])cc1]

To a solution of tert-butyl (3-aminobutyl)carbamate (1.00 g, 5.31 mmol) in dry DCM (15 mL) stirring under nitrogen was added triethylamine (0.89 mL, 6.37 mmol). The solution was cooled to 0° C., and then a solution of 4-nitrobenzenesulfonyl chloride (1.30 g, 5.84 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1 M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (3-((4-nitrophenyl)sulfonamido)butyl)carbamate as a white solid (1.95 g, 5.22 mmol, 98.3%). UPLC/ELSD: RT=0.54 min. MS (ES): m/z (MH⁺) 374.4 for C₁₅H₂₃N₃O₆S. ¹H NMR (300 MHz, CDCl₃) δ: ppm 8.07 (m, 1H), 7.78 (m, 1H), 7.68 (m, 1H), 5.23 (m, 1H), 4.81 (br. s, 1H), 3.52 (m, 1H), 3.19 (m, 1H), 3.05 (m, 1H), 1.63 (m, 2H), 1.37 (s, 9H), 0.98 (d, 3H, J=6 Hz).

### Step 2: di-tert-butyl ((butane-1,4-diylbis(azanediyl))bis(butane-3,1-diyl))dicarbamate

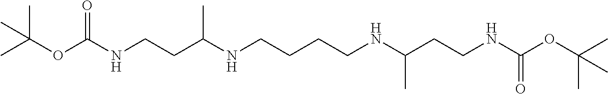 [SMILES: CC(CCNC(=O)OC(C)(C)C)NCCCCNC(C)CCNC(=O)OC(C)(C)C]

To a solution of tert-butyl (3-((4-nitrophenyl)sulfonamido)butyl)carbamate (1.95 g, 5.22 mmol) in dry DMF (20 mL) set stirring under nitrogen was added potassium carbonate (2.10 g, 15.17 mmol) and 1,4-diiodobutane (0.33 mL, 2.49 mmol). The solution was heated to 40° C. and allowed to proceed overnight. The following morning, benzyl bromide (0.25 mL, 2.06 mmol) was added, and the reaction was allowed to proceed at room temperature for 24 h. Then, thiophenol (0.98 mL, 9.57 mmol), potassium carbonate (1.03 g, 7.46 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to give an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(butane-3,1-diyl))dicarbamate as a colorless oil (0.76 g, 1.77 mmol, 71.0%). UPLC/ELSD: RT=0.42 min. MS (ES): m/z (MH⁺) 431.6 for C₂₂H₄₆N₄O₄. ¹H NMR (300 MHz, CDCl₃) δ: ppm 5.47 (m, 2H), 3.24 (br. m, 4H), 2.74 (br. m, 4H), 2.55 (m, 2H), 1.53 (m, 101H), 1.44 (s, 18H), 1.09 (d, 6H, J=6 Hz).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate

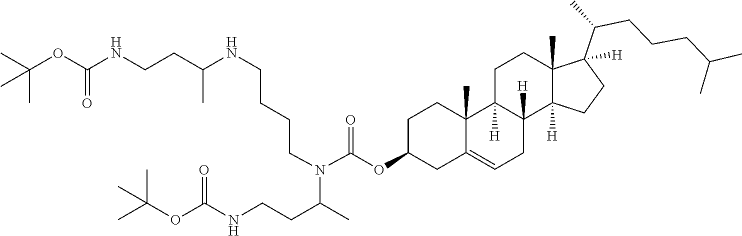

To a solution of di-tert-butyl ((butane-1,4-diylbis(azanediyl))bis(butane-3,1-diyl))dicarbamate (0.49 g, 1.15 mmol) in dry toluene (10 mL) set stirring under nitrogen was added triethylamine (0.48 mL, 3.43 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.63 g, 1.15 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to give an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate as a colorless oil (0.78 g, 0.92 mmol, 80.6%). UPLC/ELSD: RT=2.62 min. MS (ES): m/z (MH⁺) 844.3 for C₅₀H₉₀N₄O₆. ¹H NMR (300 MHz, CDCl₃) δ: ppm 5.28 (m, 1H), 3.14 (br. m, 5H), 2.59 (m, 4H), 2.25 (m, 3H), 1.90 (br. m, 7H), 1.46 (br. m, 22H), 1.34 (s, 23H), 1.09 (br. m, 28H), 0.83 (d, 5H, J=6 Hz), 0.79 (d, 7H, J=6 Hz), 0.59 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride

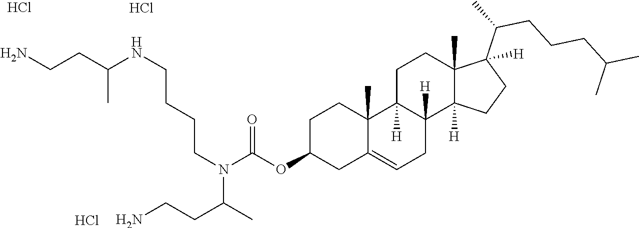

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate (0.78 g, 0.92 mmol) in isopropanol (10 mL) stirring under nitrogen was added hydrochloric acid (5 N in isopropanol, 1.85 mL, 9.23 mmol) dropwise. The solution was heated to 40° C. and allowed to proceed overnight. The following morning, dry acetonitrile (6 mL) was added to the mixture, which was sonicated and allowed to stir for an additional hour. The white solid was then filtered out of the solution, washed repeatedly with acetonitrile, and dried in vacuo to give SA74 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride as a white solid (0.57 g, 0.73 mmol, 78.8%). UPLC/ELSD: RT=1.67 min. MS (ES): m/z (MH⁺) 753.4 for C₄₀H₇₇Cl₃N₄O₂. ¹H NMR (300 MHz, CDCl₃) δ: ppm 5.42 (m, 1H), 4.49 (br. m, 1H), 4.12 (br. m, 1H), 3.45 (br. m, 1H), 3.33 (s, 2H), 3.24 (br. m, 2H), 3.13 (br. m, 4H), 2.90 (br. m, 2H), 2.41 (d, 2H, J=3 Hz), 2.32 (br. m, 1H), 1.93 (br. m, 19H), 1.43 (d, 6H, J=6 Hz), 1.31 (d, 5H, J=6 Hz), 1.08 (br. m, 12H), 0.97 (d, 4H, J=6 Hz), 0.90 (d, 6H, J=6 Hz), 0.75 (s, 3H).

### AL. Compound SA75: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-methylpropyl)(4-((3-amino-2-methylpropyl)amino)butyl)carbamate trihydrochloride

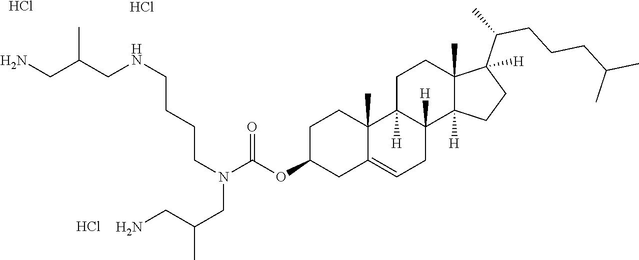

### Step 1: tert-butyl (2-methyl-3-((4-nitrophenyl)sulfonamido)propyl)carbamate

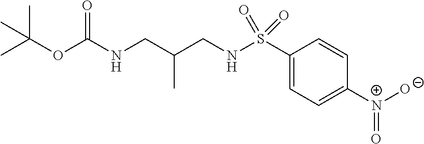 [SMILES: CC(CNC(=O)OC(C)(C)C)CNS(=O)(=O)c1ccc([N+](=O)[O-])cc1]

To a solution of tert-butyl (3-amino-2-methylpropyl)carbamate (1.00 g, 5.31 mmol) in dry DCM (15 mL) stirring under nitrogen was added triethylamine (0.89 mL, 6.37 mmol). The solution was cooled to 0° C., and then a solution of 4-nitrobenzenesulfonyl chloride (1.30 g, 5.84 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2-methyl-3-((4-nitrophenyl)sulfonamido)propyl)carbamate as a white solid (2.20 g, 5.90 mmol, quantitative). UPLC/ELSD: RT=0.58 min. MS (ES): m/z (MH⁺) 374.4 for C₁₅H₂₃N₃O₆S. ¹H NMR (300 MHz, CDCl₃) δ: ppm 8.12 (m, 1H), 7.84 (m, 1H), 7.74 (m, 1H), 6.20 (br. s, 1H), 4.82 (br. s, 1H), 3.19 (m, 1H), 3.04 (m, 3H), 1.84 (m, 1H), 1.41 (s, 9H), 0.91 (d, 3H, J=6 Hz).

### Step 2: di-tert-butyl ((butane-1,4-diylbis(azanediyl))bis(2-methylpropane-3,1-diyl))dicarbamate

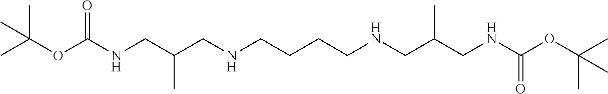 [SMILES: CC(CNCCCCNCC(C)CNC(=O)OC(C)(C)C)CNC(=O)OC(C)(C)C]

To a solution of tert-butyl (2-methyl-3-((4-nitrophenyl)sulfonamido)propyl)carbamate (2.20 g, 5.90 mmol) in dry DMF (20 mL) stirring under nitrogen was added potassium carbonate (2.37 g, 17.14 mmol) and 1,4-diiodobutane (0.37 mL, 2.81 mmol). The solution was heated to 40° C. and allowed to proceed overnight. The following morning, benzyl bromide (0.28 mL, 2.33 mmol) was added, and the reaction was allowed to proceed at room temperature for 24 h. Then, thiophenol (1.11 mL, 10.82 mmol), potassium carbonate (1.17 g, 8.43 mmol), and an additional 5 mL of dry DMF were added, and the reaction was allowed to proceed overnight again. The following morning, salts were removed from the supernatant via multiple rounds of centrifugation and rinsing with DMF. The combined supernatants were concentrated in vacuo to an oil which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2-methylpropane-3,1-diyl))dicarbamate as a colorless oil (0.85 g, 1.97 mmol, 70.0%). UPLC/ELSD: RT=0.43 min. MS (ES): m/z (MH⁺) 431.6 for C₂₂H₄₆N₄O₄. ¹H NMR (300 MHz, CDCl₃) δ: ppm 5.76 (m, 1H), 2.93 (m, 2H), 2.73 (m, 2H), 2.28 (m, 8H), 1.56 (m, 4H), 1.28 (s, 4H), 1.18 (s, 17H), 0.66 (d, 6H, J=6 Hz).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-methylpropyl)(4-((3-((tert-butoxycarbonyl)amino)-2-methylpropyl)amino)butyl)carbamate

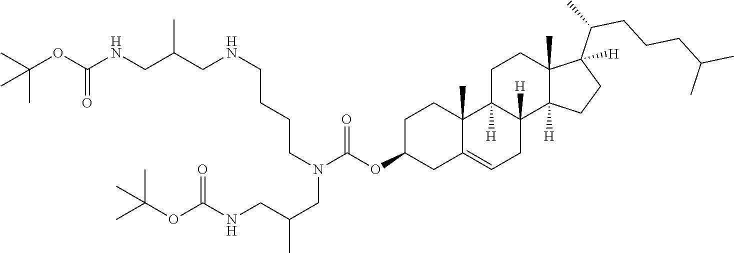

To a solution of di-tert-butyl ((butane-1,4-diylbis(azanediyl))bis(2-methylpropane-3,1-diyl))dicarbamate (1.06 g, 2.45 mmol) in dry toluene (20 mL) stirring under nitrogen was added triethylamine (0.86 mL, 6.13 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (1.13 g, 2.04 mmol) was added and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-methylpropyl)(4-((3-((tert-butoxycarbonyl)amino)-2-methylpropyl)amino)butyl)carbamate as a colorless oil (1.08 g, 1.28 mmol, 62.8%). UPLC/ELSD: RT=2.52 min. MS (ES): m/z (MH⁺) 844.3 for C₅₀H₉₀N₄O₆. ¹H NMR (300 MHz, CDCl₃) δ: ppm 5.29 (m, 1H), 4.42 (br. m, 1H), 3.07 (br. m, 5H), 2.87 (m, 3H), 2.51 (m, 4H), 2.25 (br. m, 2H), 1.79 (br. m, 7H), 1.46 (m, 8H), 1.34 (s, 18H), 1.05 (br. m, 10H), 0.94 (s, 5H), 0.82 (m, 14H), 0.59 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-methylpropyl)(4-((3-amino-2-methylpropyl)amino)butyl)carbamate trihydrochloride

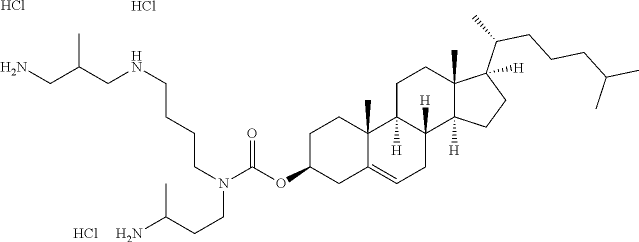

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-methylpropyl)(4-((3-((tert-butoxycarbonyl)amino)-2-methylpropyl)amino)butyl)carbamate (1.08 g, 1.28 mmol) in isopropanol (10 mL) set stirring under nitrogen was added hydrochloric acid (5 N in isopropanol, 2.57 mL, 12.83 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give SA75 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-methylpropyl)(4-((3-amino-2-methylpropyl)amino)butyl)carbamate trihydrochloride as a white solid (0.66 g, 0.85 mmol, 66.1%). UPLC/ELSD: RT=1.59 min. MS (ES): m/z (MH⁺) 753.4 for C₄₀H₇₇Cl₃N₄O₂. ¹H NMR (300 MHz, CDCl₃) δ: ppm 5.42 (m, 1H), 4.46 (br. m, 1H), 3.33 (br. m, 4H), 3.12 (br. m, 5H), 2.95 (m, 4H), 2.40 (d, 3H, J=9 Hz), 1.75 (br. m, 19H), 1.20 (br. m, 9H), 1.08 (m, 8H), 0.97 (d, 4H, J=6 Hz), 0.89 (d, 6H, J=6 Hz), 0.75 (s, 3H).

### AM. Compound SA76: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride

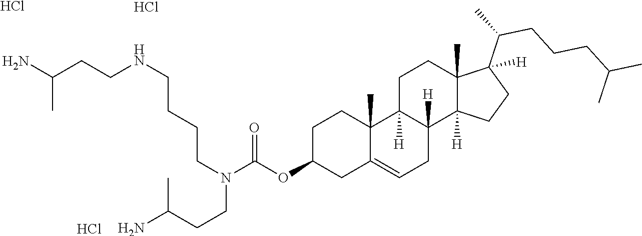

### Step 1: tert-butyl (4-((4-nitrophenyl)sulfonamido)butan-2-yl)carbamate

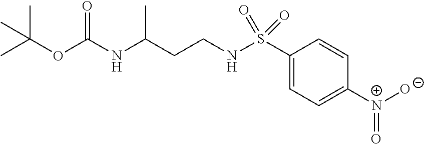 [SMILES: CC(CCNS(=O)(=O)c1ccc([N+](=O)[O-])cc1)NC(=O)OC(C)(C)C]

To a solution of tert-butyl (4-aminobutan-2-yl)carbamate (1.00 g, 5.31 mmol) in dry DCM (15 mL) stirring under nitrogen was added triethylamine (0.89 mL, 6.37 mmol). The solution was cooled to 0° C., and then a solution of 4-nitrobenzenesulfonyl chloride (1.30 g, 5.84 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (4-((4-nitrophenyl)sulfonamido)butan-2-yl)carbamate as a white solid (1.47 g, 3.94 mmol, 74.1%). UPLC/ELSD: RT=0.61 min. MS (ES): m/z (MH⁺) 374.4 for C₁₅H₂₃N₃O₆S. ¹H NMR (300 MHz, CDCl₃) δ: ppm 8.00 (m, 1H), 7.81 (m, 2H), 7.73 (m, 2H), 6.15 (br. s, 1H), 4.27 (br. s, 1H), 3.64 (br. s, 1H), 3.19 (br. s, 1H), 2.95 (br. s, 1H), 1.64 (m, 1H), 1.30 (s, 10H), 1.00 (d, 3H, J=6 Hz).

### Step 2: di-tert-butyl ((butane-1,4-diylbis(azanediyl))bis(butane-4,2-diyl))dicarbamate

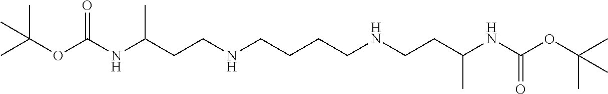 [SMILES: CC(CCNCCCCNCCC(C)NC(=O)OC(C)(C)C)NC(=O)OC(C)(C)C]

To a solution of tert-butyl (4-((4-nitrophenyl)sulfonamido)butan-2-yl)carbamate (1.47 g, 3.94 mmol) in dry DMF (20 mL) set stirring under nitrogen was added potassium carbonate (1.58 g, 11.44 mmol) and 1,4-diiodobutane (0.25 mL, 1.88 mmol). The solution was heated to 40° C. and allowed to proceed overnight. The following morning, benzyl bromide (0.19 mL, 1.56 mmol) was added, and the reaction was allowed to proceed at room temperature for 24 h. Then, thiophenol (0.74 mL, 7.22 mmol), potassium carbonate (0.78 g, 5.62 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(butane-4,2-diyl))dicarbamate as a colorless oil (0.17 g, 0.40 mmol, 21.2%). UPLC/ELSD: RT=0.45 min. MS (ES): m/z (MH⁺) 431.6 for C₂₂H₄₆N₄O₄. ¹H NMR (300 MHz, CDCl₃) δ: ppm 4.94 (m, 1H), 3.66 (m, 5H), 2.66 (m, 8H), 1.66 (m, 8H), 1.39 (s, 18H), 1.10 (d, 6H, J=9 Hz).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate

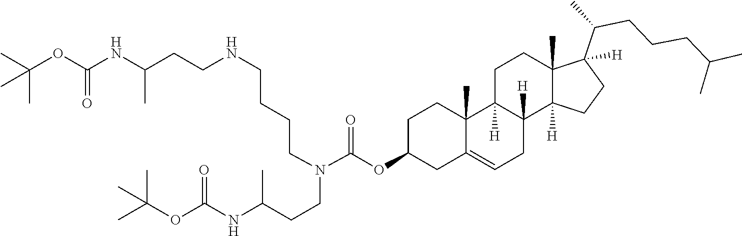

To a solution of di-tert-butyl ((butane-1,4-diylbis(azanediyl))bis(butane-4,2-diyl))dicarbamate (0.17 g, 0.40 mmol) in dry toluene (5 mL) set stirring under nitrogen was added triethylamine (0.15 mL, 1.08 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.20 g, 0.36 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate as a colorless oil (0.13 g, 0.16 mmol, 43.0%). UPLC/ELSD: RT=2.77 min. MS (ES): m/z (MH⁺) 844.3 for C₅₀H₉₀N₄O₆. ¹H NMR (300 MHz, CDCl₃) δ: ppm 5.36 (m, 1H), 4.89 (m, 1H), 4.50 (m, 2H), 3.67 (br. m, 2H), 3.22 (br. m, 4H), 2.59 (m, 4H), 2.33 (m, 2H), 1.98 (br. m, 6H), 1.54 (br. m, 15H), 1.43 (s, 22H), 1.34 (m, 5H), 1.14 (m, 14H), 1.02 (s, 7H), 0.82 (m, 14H), 0.91 (d, 4H, J=6 Hz), 0.86 (d, 6H, J=6 Hz), 0.67 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride

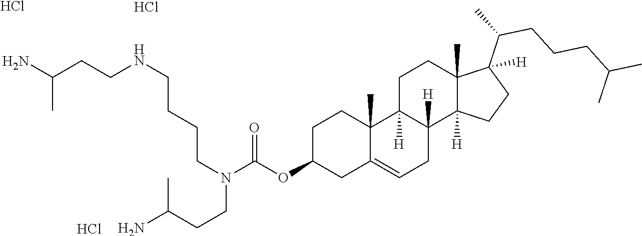

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate (0.13 g, 0.16 mmol) in isopropanol (10 mL) set stirring under nitrogen was added hydrochloric acid (5 N in isopropanol, 0.31 mL, 1.55 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give SA76 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride as a white solid (0.06 g, 0.07 mmol, 47.9%). UPLC/ELSD: RT=1.49 min. MS (ES): m/z (MH⁺) 753.4 for C₄₀H₇₇Cl₃N₄O₂. ¹H NMR (300 MHz, CDCl₃) δ: ppm 5.42 (m, 1H), 4.48 (br. m, 1H), 3.47 (br. m, 2H), 3.33 (br. m, 7H), 3.17 (m, 4H), 2.40 (d, 2H, J=9 Hz), 2.05 (br. m, 8H), 1.62 (br. m, 10H), 1.39 (d, 8H, J=9 Hz), 1.16 (d, 7H, J=6 Hz), 1.08 (br. m, 5H), 0.98 (d, 3H, J=6 Hz), 0.91 (d, 5H, J=6 Hz), 0.74 (s, 3H).

### AN. Compound SA77: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-dimethylpropyl)(4-((3-amino-2,2-dimethylpropyl)amino)butyl)carbamate trihydrochloride

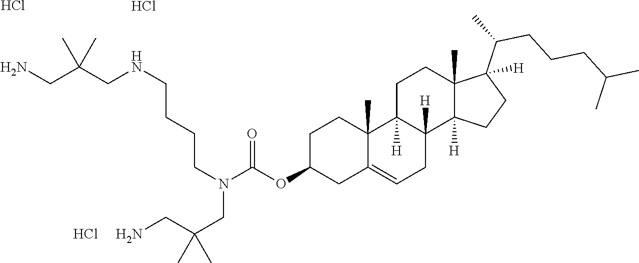

### Step 1: tert-butyl (2,2-dimethyl-3-((4-nitrophenyl)sulfonamido)propyl)carbamate

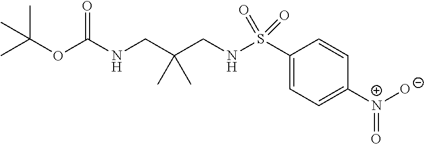 [SMILES: CC(C)(CNC(=O)OC(C)(C)C)CNS(=O)(=O)c1ccc([N+](=O)[O-])cc1]

To a solution of tert-butyl (3-amino-2,2-dimethylpropyl)carbamate (1.00 g, 4.94 mmol) in dry DCM (15 mL) set stirring under nitrogen was added triethylamine (0.83 mL, 5.93 mmol). The solution was cooled to 0° C., and then a solution of 4-nitrobenzenesulfonyl chloride (1.20 g, 5.44 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2,2-dimethyl-3-((4-nitrophenyl)sulfonamido)propyl)carbamate as a white solid (2.00 g, 5.15 mmol, 104.1%). UPLC/ELSD: RT=0.85 min. MS (ES): m/z (MH⁺) 388.4 for C₁₆H₂₅N₃O₆S. ¹H NMR (300 MHz, CDCl₃) δ: ppm 8.10 (m, 1H), 7.82 (m, 1H), 7.73 (m, 2H), 6.54 (br. s, 1H), 4.86 (br. s, 1H), 2.99 (d, 2H, J=6 Hz), 2.81 (d, 2H, J=9 Hz), 1.41 (s, 9H), 0.90 (s, 6H).

### Step 2: di-tert-butyl ((butane-1,4-diylbis(azanediyl))bis(2,2-dimethylpropane-3,1-diyl))dicarbamate

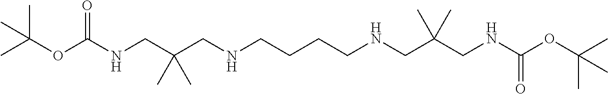 [SMILES: CC(C)(CNCCCCNCC(C)(C)CNC(=O)OC(C)(C)C)CNC(=O)OC(C)(C)C]

To a solution of tert-butyl (2,2-dimethyl-3-((4-nitrophenyl)sulfonamido)propyl)carbamate (2.00 g, 5.15 mmol) in dry DMF (20 mL) stirring under nitrogen was added potassium carbonate (2.07 g, 14.96 mmol) and 1,4-diiodobutane (0.32 mL, 2.45 mmol). The solution was heated to 40° C. and allowed to proceed overnight. The following morning, benzyl bromide (0.24 mL, 2.04 mmol) was added, and the reaction was allowed to proceed at room temperature for 24 h. Then, thiophenol (0.97 mL, 9.44 mmol), potassium carbonate (1.02 g, 7.36 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to give an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2,2-dimethylpropane-3,1-diyl))dicarbamate as a colorless oil (0.63 g, 1.38 mmol, 56.3%). UPLC/ELSD: RT=0.46 min. MS (ES): m/z (MH⁺) 459.7 for C₂₄H₅₀N₄O₄. ¹H NMR (300 MHz, CDCl₃) δ: ppm 5.84 (m, 2H), 2.82 (d, 4H, J=6 Hz), 2.39 (br. s, 4H), 2.22 (s, 4H), 1.33 (br. m, 4H), 1.24 (s, 18H), 0.70 (s, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-dimethylpropyl)(4-((3-((tert-butoxycarbonyl)amino)-2,2-dimethylpropyl)amino)butyl)carbamate

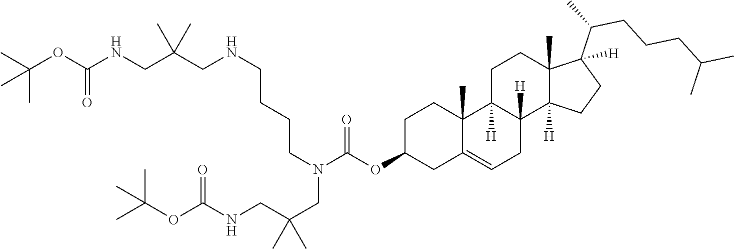

To a solution of di-tert-butyl ((butane-1,4-diylbis(azanediyl))bis(2,2-dimethylpropane-3,1-diyl))dicarbamate (0.66 g, 1.43 mmol) in dry toluene (10 mL) set stirring under nitrogen was added triethylamine (0.55 mL, 3.90 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.72 g, 1.30 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-dimethylpropyl)(4-((3-((tert-butoxycarbonyl)amino)-2,2-dimethylpropyl)amino)butyl)carbamate as a colorless oil (0.39 g, 0.45 mmol, 34.7%). UPLC/ELSD: RT=2.77 min. MS (ES): m/z (MH⁺) 872.3 for C₅₂H₉₄N₄O₆. ¹H NMR (300 MHz, CDCl₃) δ: ppm 5.38 (m, 1H), 5.09 (m, 1H), 4.23 (m, 1H), 2.92 (br. m, 2H), 2.72 (m, 6H), 2.27 (m, 2H), 2.10 (m, 4H), 1.70 (m, 5H), 1.28 (br. m, 8H), 1.14 (s, 21H), 1.05 (m, 4H), 0.85 (m, 8H), 0.74 (s, 7H), 0.60 (br. m, 22H), 0.39 (s, 4H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-dimethylpropyl)(4-((3-amino-2,2-dimethylpropyl)amino)butyl)carbamate trihydrochloride

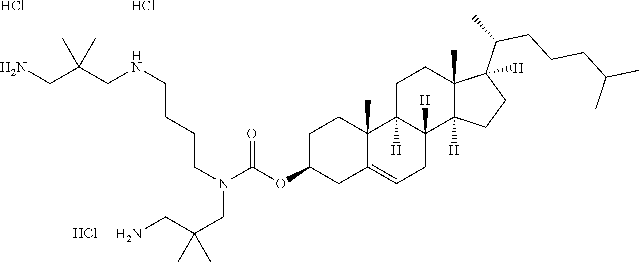

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-dimethylpropyl)(4-((3-((tert-butoxycarbonyl)amino)-2,2-dimethylpropyl)amino)butyl)carbamate (0.39 g, 0.45 mmol) in isopropanol (10 mL) stirring under nitrogen was added hydrochloric acid (5 N in isopropanol, 0.90 mL, 4.51 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-dimethylpropyl)(4-((3-amino-2,2-dimethylpropyl)amino)butyl)carbamate trihydrochloride as a white solid SA77 (0.21 g, 0.26 mmol, 58.0%). UPLC/ELSD: RT=1.49 min. MS (ES): m/z (MH⁺) 781.5 for C₄₂H₈₁Cl₃N₄O₂. ¹H NMR (300 MHz, CDCl₃) δ: ppm 5.42 (m, 1H), 4.51 (br. m, 1H), 3.33 (br. m, 5H), 3.08 (d, 6H, J=9 Hz), 2.73 (m, 2H), 2.42 (d, 2H, J=6 Hz), 1.75 (br. m, 16H), 1.39 (br. m, 4H), 1.22 (br. m, 11H), 1.09 (br. m, 11H), 0.97 (d, 4H, J=6 Hz), 0.90 (d, 6H, J=6 Hz), 0.74 (s, 3H).

### AO. Compound SA78: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride

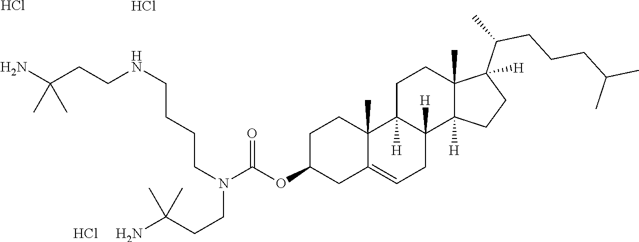

### Step 1: tert-butyl (2-methyl-4-((4-nitrophenyl)sulfonamido)butan-2-yl)carbamate

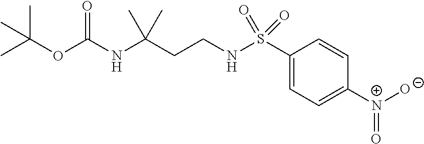 [SMILES: CC(C)(CCNS(=O)(=O)c1ccc([N+](=O)[O-])cc1)NC(=O)OC(C)(C)C]

To a solution of tert-butyl (4-amino-2-methylbutan-2-yl)carbamate (1.00 g, 4.94 mmol) in dry DCM (15 mL) stirring under nitrogen was added triethylamine (0.83 mL, 5.93 mmol). The solution was cooled to 0° C., and then a solution of 4-nitrobenzenesulfonyl chloride (1.20 g, 5.44 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2-methyl-4-((4-nitrophenyl)sulfonamido)butan-2-yl)carbamate as a white solid (1.86 g, 4.79 mmol, 96.9%). UPLC/ELSD: RT=0.69 min. MS (ES): m/z (MH⁺) 388.4 for C₁₆H₂₅N₃O₆S. ¹H NMR (300 MHz, CDCl₃) δ: ppm 8.10 (m, 1H), 7.84 (m, 1H), 7.75 (m, 2H), 5.46 (br. s, 1H), 4.47 (s, 1H), 3.15 (q, 2H), 1.94 (t, 2H), 1.39 (s, 9H), 1.23 (s, 6H).

### Step 2: di-tert-butyl ((butane-1,4-diylbis(azanediyl))bis(2-methylbutane-4,2-diyl))dicarbamate

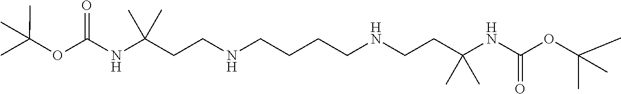 [SMILES: CC(C)(CCNCCCCNCCC(C)(C)NC(=O)OC(C)(C)C)NC(=O)OC(C)(C)C]

To a solution of tert-butyl (2-methyl-4-((4-nitrophenyl)sulfonamido)butan-2-yl)carbamate (1.86 g, 4.79 mmol) in dry DMF (20 mL) set stirring under nitrogen was added potassium carbonate (1.92 g, 13.92 mmol) and 1,4-diiodobutane (0.30 mL, 2.28 mmol). The solution was heated to 40° C. and allowed to proceed overnight. The following morning, benzyl bromide (0.23 mL, 1.89 mmol) was added, and the reaction was allowed to proceed at room temperature for 24 h. Then, thiophenol (0.90 mL, 8.78 mmol), potassium carbonate (0.95 g, 6.84 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2-methylbutane-4,2-diyl))dicarbamate as a colorless oil (0.57 g, 1.24 mmol, 54.3%). UPLC/ELSD: RT=0.46 min. MS (ES): m/z (MH⁺) 459.7 for C₂₄H₅₀N₄O₄. ¹H NMR (300 MHz, CDCl₃) δ: ppm 5.94 (m, 2H), 2.61 (m, 8H), 1.92 (br. m, 2H), 1.61 (m, 4H), 1.45 (br. m, 4H), 1.32 (s, 18H), 1.21 (s, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate

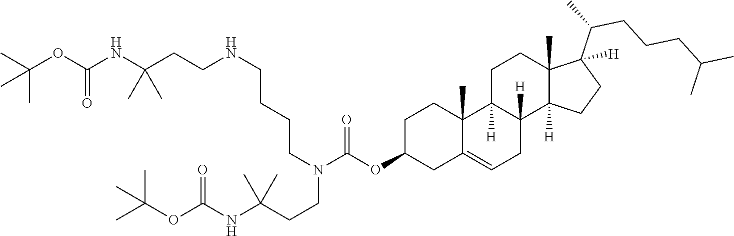

To a solution of di-tert-butyl ((butane-1,4-diylbis(azanediyl))bis(2-methylbutane-4,2-diyl))dicarbamate (0.68 g, 1.48 mmol) in dry toluene (10 mL) stirring under nitrogen was added triethylamine (0.57 mL, 4.03 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.74 g, 1.34 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate as a colorless oil (0.57 g, 0.65 mmol, 48.3%). UPLC/ELSD: RT=2.65 min. MS (ES): m/z (MH⁺) 872.3 for C₅₂H₉₄N₄O₆. ¹H NMR (300 MHz, CDCl₃) δ: ppm 5.99 (m, 1H), 5.27 (m, 1H), 4.40 (m, 2H), 3.11 (br. m, 4H), 2.59 (t, 2H), 2.50 (t, 21), 2.25 (m, 2H), 1.77 (m, 7H), 1.58 (m, 2H), 1.44 (br. m, 12H), 1.32 (s, 18H), 1.20 (d, 16H, J=9 Hz), 1.01 (m, 9H), 0.92 (s, 6H), 0.82 (d, 4H, J=6 Hz), 0.75 (d, 6H, J=9 Hz), 0.57 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride

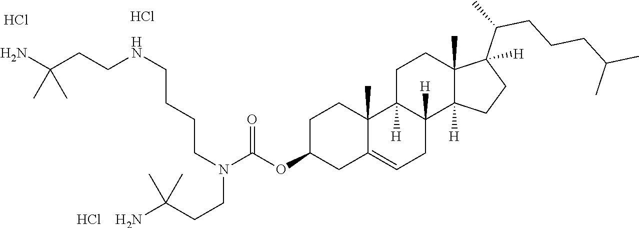

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate (0.57 g, 0.65 mmol) in isopropanol (10 mL) set stirring under nitrogen was added hydrochloric acid (5 N in isopropanol, 1.30 mL, 6.50 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give SA78 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride as a white solid (0.35 g, 0.44 mmol, 67.2%). UPLC/ELSD: RT=1.50 min. MS (ES): m/z (MH⁺) 781.5 for C₄₂H₈₁Cl₃N₄O₂. ¹H NMR (300 MHz, CDCl₃) δ: ppm 5.43 (m, 1H), 4.44 (br. m, 1H), 3.33 (br. m, 5H), 3.15 (br. m, 3H), 2.38 (m, 2H), 2.16 (br. m, 8H), 1.74 (br. m, 10H), 1.43 (br. m, 14H), 1.17 (d, 9H, J=6 Hz), 1.08 (br. m, 5H), 0.98 (d, 4H, J=6 Hz), 0.90 (d, 6H, J=6 Hz), 0.74 (s, 3H).

### AP. Compound SA79: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride

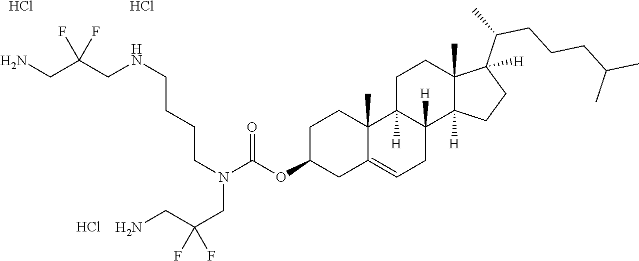

### Step 1: tert-butyl (2,2-difluoro-3-((4-nitrophenyl)sulfonamido)propyl)carbamate

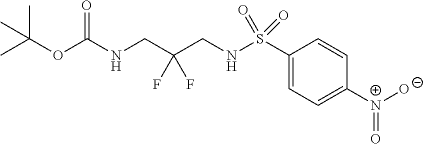 [SMILES: CC(C)(C)OC(=O)NCC(F)(F)CNS(=O)(=O)c1ccc([N+](=O)[O-])cc1]

To a solution of tert-butyl (3-amino-2,2-difluoropropyl)carbamate (0.95 g, 4.52 mmol) in dry DCM (15 mL) set stirring under nitrogen was added triethylamine (0.76 mL, 5.42 mmol). The solution was cooled to 0° C., and then a solution of 4-nitrobenzenesulfonyl chloride (1.10 g, 4.97 mmol) in 5 mL dry DCM was added dropwise over 30 minutes. The reaction was allowed to proceed at 0° C. for an hour, and then at room temperature for an additional three hours. Then the mixture was diluted with an additional 10 mL DCM, washed with 1 M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2,2-difluoro-3-((4-nitrophenyl)sulfonamido)propyl)carbamate as a white solid (1.66 g, 4.19 mmol, 92.6%). UPLC/ELSD: RT=0.61 min. MS (ES): m/z (MH⁺) 396.4 for C₁₄H₁₉F₂N₃O₆S. ¹H NMR (300 MHz, CDCl₃) δ: ppm 8.15 (m, 1H), 7.87 (m, 1H), 7.73 (m, 2H), 6.68 (br. s, 1H), 5.02 (br. s, 1H), 3.55 (br. m, 4H), 1.45 (s, 9H).

### Step 2: di-tert-butyl ((butane-1,4-diylbis(azanediyl))bis(2,2-difluoropropane-3,1-diyl))dicarbamate

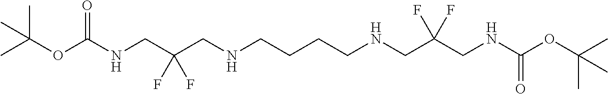 [SMILES: CC(C)(C)OC(=O)NCC(F)(F)CNCCCCNCC(F)(F)CNC(=O)OC(C)(C)C]

To a solution of tert-butyl (2,2-difluoro-3-((4-nitrophenyl)sulfonamido)propyl)carbamate (1.65 g, 4.18 mmol) in dry DMF (20 mL) set stirring under nitrogen was added potassium carbonate (1.68 g, 12.14 mmol) and 1,4-diiodobutane (0.26 mL, 1.99 mmol). The solution was heated to 40° C. and allowed to proceed overnight. The following morning, benzyl bromide (0.20 mL, 1.65 mmol) was added, and the reaction was allowed to proceed at room temperature for 24 h. Then thiophenol (0.78 mL, 7.67 mmol), potassium carbonate (0.83 g, 5.97 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2,2-difluoropropane-3,1-diyl))dicarbamate as a colorless oil (0.50 g, 1.07 mmol, 53.3%). UPLC/ELSD: RT=0.39 min. MS (ES): m/z (MH⁺) 475.5 for C₂₀H₃₈F₄N₄O₄. ¹H NMR (300 MHz, CDCl₃) δ: ppm 5.14 (m, 2H), 3.25 (m, 4H), 2.62 (m, 4H), 2.33 (br. m, 4H), 1.18 (br. m, 4H), 1.12 (s, 18H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate

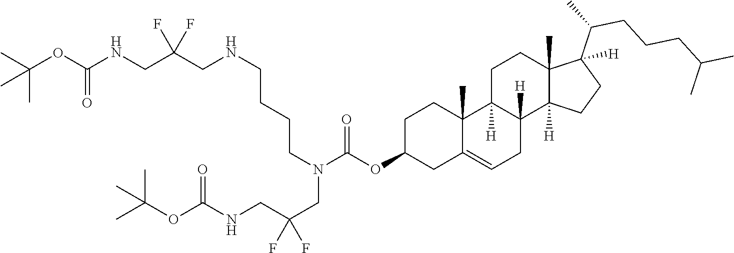

To a solution of di-tert-butyl ((butane-1,4-diylbis(azanediyl))bis(2,2-difluoropropane-3,1-diyl))dicarbamate (0.64 g, 1.35 mmol) in dry toluene (10 mL) stirring under nitrogen was added triethylamine (0.52 mL, 3.68 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.68 g, 1.23 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate as a colorless oil (0.12 g, 0.13 mmol, 10.7%). UPLC/ELSD: RT=2.63 min. MS (ES): m/z (MH⁺) 888.2 for C₄₈H₈₂F₄N₄O₆. ¹H NMR (300 MHz, CDCl₃) δ: ppm 5.61 (m, 1H), 5.31 (m, 1H), 4.98 (m, 1H), 4.55 (br. m, 1H), 3.63 (br. m, 5H), 3.32 (m, 2H), 2.97 (t, 2H), 2.69 (t, 2H), 2.36 (m, 2H), 2.05 (br. m, 5H), 1.60 (br. m, 5H), 1.46 (s, 21H), 1.15 (m, 6H), 1.04 (s, 5H), 0.93 (d, 3H, J=6 Hz), 0.89 (d, 5H, J=6 Hz), 0.69 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride

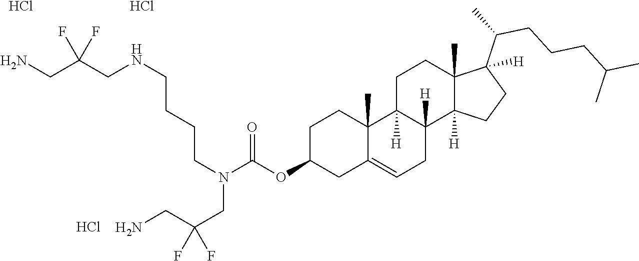

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate (0.12 g, 0.13 mmol) in isopropanol (10 mL) stirring under nitrogen was added hydrochloric acid (5 N in isopropanol, 0.26 mL, 1.31 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride as a white solid SA79 (0.05 g, 0.06 mmol, 42.8%). UPLC/ELSD: RT=1.52 min. MS (ES): m/z (MH⁺) 797.4 for C₃₈H₆₉Cl₃F₄N₄O₂. ¹H NMR (300 MHz, CDCl₃) δ: ppm 5.43 (m, 1H), 4.49 (br. m, 1H), 3.94 (br. m, 7H), 3.77 (br. m, 4H), 3.33 (m, 3H), 3.22 (m, 2H), 2.42 (m, 2H), 1.76 (br. m, 23H), 1.18 (d, 12H, J=6 Hz), 1.08 (br. m, 6H), 0.98 (d, 4H, J=9 Hz), 0.91 (d, 6H, J=6 Hz), 0.75 (s, 3H).

### AQ. Compound SA81: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl bis(6-aminohexyl)carbamate dihydrochloride

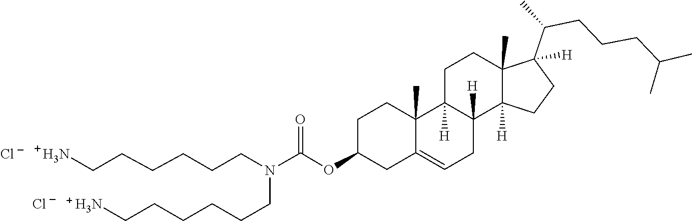

### Step 1: tert-Butyl N-{6-[benzyl({6-[(tert-butoxycarbonyl)amino]hexyl})amino]hexyl}carbamate

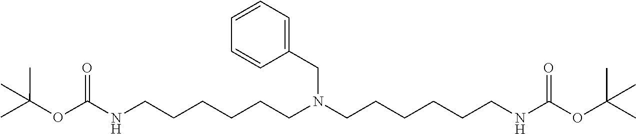 [SMILES: CC(C)(C)OC(=O)NCCCCCCN(CCCCCCNC(=O)OC(C)(C)C)Cc1ccccc1]

To a suspension of tert-butyl N-(6-bromohexyl)carbamate (2.694 g, 9.613 mmol), potassium carbonate (1.898 g, 13.73 mmol), and potassium iodide (0.152 g, 0.915 mmol) in dimethylformamide (7.5 mL) was added benzylamine (0.50 mL, 4.6 mmol). The reaction mixture stirred at 50° C. and was monitored by LCMS. At 23.5 h, the reaction mixture was cooled to rt and then diluted with methyl tert-butyl ether (150 mL). The diluted mixture was washed with water (4×) and brine, dried over Na₂SO₄, and concentrated. The crude material was purified via silica gel chromatography (20-70% methyl tert-butyl ether in hexanes) to afford tert-butyl N-{6-[benzyl({6-[(tert-butoxycarbonyl)amino]hexyl})amino]hexyl}carbamate (2.144 g, 4.239 mmol, 92.6%) as a clear oil. UPLC/ELSD: RT=0.87 min. MS (ES): m/z=506.70 [M+H]⁺ for C₂₉H₅₁N₃O₄; ¹H NMR (300 MHz, CDCl₃): δ 7.17-7.37 (m, 5H), 4.50 (br. s, 2H), 3.52 (s, 2H), 3.08 (dt, 4H, J=6.5, 6.2 Hz), 2.37 (t, 4H, J=7.2 Hz), 1.37-1.51 (m, 8H), 1.44 (s, 18H), 1.22-1.33 (m, 8H).

### Step 2: tert-Butyl N-[6-([6-[(tert-butoxycarbonyl)amino]hexyl]amino)hexyl]carbamate

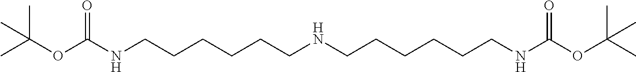 [SMILES: CC(C)(C)OC(=O)NCCCCCCNCCCCCCNC(=O)OC(C)(C)C]

Tert-butyl N-{6-[benzyl({6-[(tert-butoxycarbonyl)amino]hexyl})amino]hexyl}carbamate (2.12 g, 4.192 mmol) and 10% Pd/C (0.892 g, 0.419 mmol) were combined in ethanol (35 mL) and then stirred under a balloon of H₂ at rt. The reaction was monitored by TLC. At 19 h, the reaction mixture was filtered through a pad of Celite rinsing with ethyl acetate. The filtrate was concentrated, taken up in ethyl acetate, filtered through a 0.45 μm PTFE frit, and concentrated to afford tert-butyl N-[6-({6-[(tert-butoxycarbonyl)amino]hexyl}amino)hexyl]carbamate (1.537 g, 3.698 mmol, 88.2%) as an off-white solid. UPLC/ELSD: RT=0.62 min. MS (ES): m/z=416.60 [M+H]⁺ for C₂₂H₄₅N₃O₄; ¹H NMR (300 MHz, CDCl₃): δ 4.51 (br. s, 2H), 3.10 (dt, 4H, J=6.4, 6.4 Hz), 2.57 (t, 4H, J=7.1 Hz), 1.13-1.57 (br. m, 17H), 1.44 (s, 18H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate

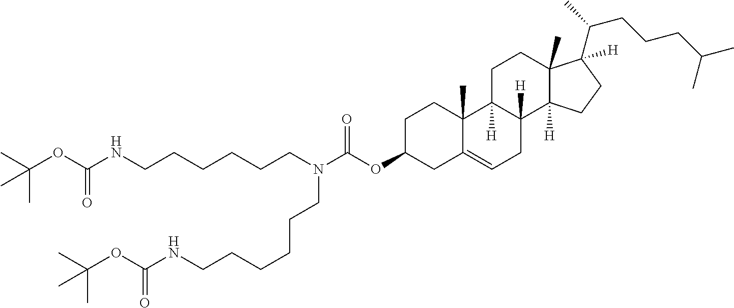

Cholesterol 4-nitrophenyl carbonate (0.200 g, 0.362 mmol), tert-butyl N-[6-({6-[(tert-butoxycarbonyl)amino]hexyl}amino)hexyl]carbamate (0.188 g, 0.453 mmol), and triethylamine (0.15 mL, 1.08 mmol) were combined in toluene (3.5 mL). The reaction mixture stirred at 90° C. and was monitored by LCMS. At 17 h, the reaction mixture was cooled to rt, diluted with dichloromethane (30 mL), and washed with 5% aq. NaHCO₃ solution (3×25 mL). The organics were passed through a hydrophobic frit, dried over Na₂SO₄, and concentrated. The crude material was purified via silica gel chromatography (0-50% ethyl acetate in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate (0.286 g, 0.345 mmol, 95.3%) as a clear oil.

UPLC/ELSD: RT=3.53 min. MS (ES): m/z=728.94 [(M+H)—(CH₃)₂C═CH₂— CO₂]⁺ for C₅₀H₈₉N₃O₆; ¹H NMR (300 MHz, CDCl₃): δ 5.33-5.42 (m, 1H), 4.41-4.64 (m, 3H), 2.99-3.28 (m, 8H), 2.20-2.41 (m, 2H), 1.75-2.08 (m, 5H), 0.93-1.67 (br. m, 37H), 1.44 (s, 18H), 1.02 (s, 3H), 0.91 (d, 3H, J=6.4 Hz), 0.87 (d, 3H, J=6.6 Hz), 0.86 (d, 3H, J=6.5 Hz), 0.68 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(6-aminohexyl)carbamate dihydrochloride

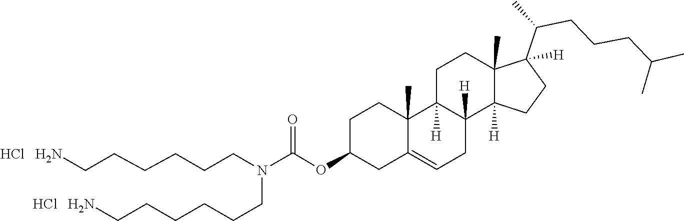

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate (0.280 g, 0.338 mmol) in isopropanol (3.5 mL) was added 5-6 N HCl in isopropanol (0.52 mL). The reaction mixture stirred at 40° C. and was monitored by LCMS. At 18 h, the reaction mixture was cooled to rt and then acetonitrile (10.5 mL) was added. The suspension was cooled to 0° C. in an ice bath. Solids were then collected by vacuum filtration rinsing with cold 3:1 acetonitrile/isopropanol to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(6-aminohexyl)carbamate dihydrochloride (0.179 g, 0.251 mmol, 74.4%) as a white solid).

UPLC/ELSD: RT=2.09 min. MS (ES): m/z=335.49 [(M+2H)+CH₃CN]²⁺ for C₄₀H₇₃N₃O₂; ¹H NMR (300 MHz, CDCl₃): δ 5.36-5.44 (m, 1H), 4.33-4.48 (m, 1H), 3.25 (t, 4H, J=7.2 Hz), 2.92 (t, 4H, J=7.6 Hz), 2.24-2.39 (m, 2H), 1.79-2.12 (m, 5H), 0.97-1.73 (br. m, 37H), 1.05 (s, 3H), 0.95 (d, 3H, J=6.4 Hz), 0.88 (d, 3H, J=6.5 Hz), 0.88 (d, 3H, J=6.6 Hz), 0.73 (s, 3H).

### AR. Compound SA82: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-aminohexyl)carbamate dihydrochloride

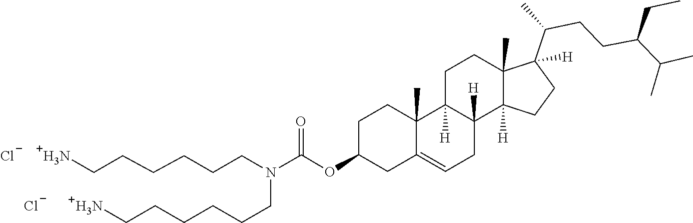

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate

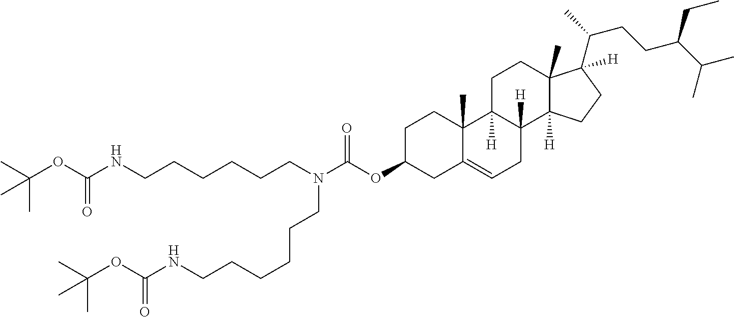

β-Sitosterol 4-nitrophenyl carbonate (0.200 g, 0.345 mmol), tert-butyl N-[6-({6-[(tert-butoxycarbonyl)amino]hexyl}amino)hexyl]carbamate (0.179 g, 0.431 mmol), triethylamine (0.15 mL, 1.1 mmol) were combined in toluene (3.5 mL). The reaction mixture stirred at 90° C. and was monitored by LCMS. At 17 h, the reaction mixture was cooled to rt, diluted with dichloromethane (ca. 30 mL) and washed with 5% aq. NaHCO₃ solution (3×25 mL). The combined washes were extracted with dichloromethane (25 mL). The combined organics were passed through a hydrophobic frit, dried over Na₂SO₄, and concentrated. The crude material was purified via silica gel chromatography (0-40% ethyl acetate in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate (0.259 g, 0.302 mmol, 87.7%) as a clear oil. UPLC/ELSD: RT=3.63 min. MS (ES): m/z=857.26 [M+H]⁺ for C₅₂H₉₃N₃O₆; ¹H NMR (300 MHz, CDCl₃): δ 5.33-5.42 (m, 1H), 4.41-4.61 (m, 3H), 3.02-3.28 (m, 8H), 2.19-2.42 (m, 2H), 1.76-2.08 (m, 5H), 0.88-1.73 (br. m, 38H), 1.44 (s, 18H), 1.02 (s, 3H), 0.92 (d, 3H, J=6.4 Hz), 0.79-0.89 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-JH-cyclopenta[a]phenanthren-3-yl bis(6-aminohexyl)carbamate dihydrochloride

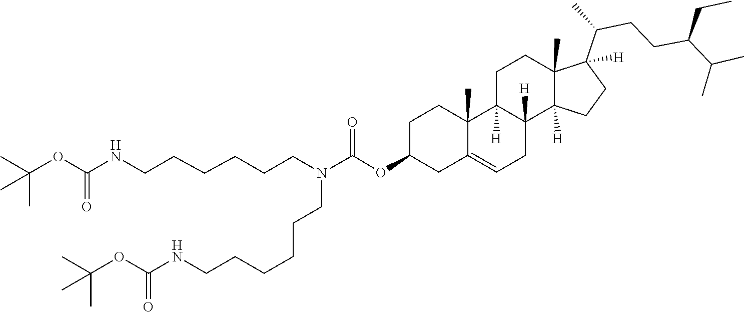

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate (0.256 g, 0.299 mmol) in isopropanol (3.2 mL) was added 5-6 N HCl in isopropanol (0.45 mL). The reaction mixture stirred at 40° C. and was monitored by LCMS. At 18 h, the reaction mixture was cooled to rt and then acetonitrile (9.6 mL) was added. The suspension was cooled to 0° C. in an ice bath, and solids were collected by vacuum filtration rinsing with cold 3:1 acetonitrile/isopropanol to afford (3S,8S,9S,10R,13R,14S,17R)-17-((2R,5R)-5-ethyl-6-methylheptan-2-yl)-10,13-dimethyl-2,4,7,8,9,10,11,12,13,14,15,16,17-tetradecahydro-1H-cyclopenta[a]phenanthren-3-yl bis(6-aminohexyl)carbamate dihydrochloride (0.167 g, 0.216 mmol, 72.1%) as a white solid. UPLC/ELSD: RT=2.23 min. MS (ES): m/z=349.94 [(M+2H)+CH₃CN]²⁺ for C₄₂H₇₇N₃O₂; ¹H NMR (300 MHz, CD₃OD): δ 5.36-5.54 (m, 1H), 4.33-4.48 (m, 1H), 3.25 (t, 4H, J=7.1 Hz), 2.92 (t, 4H, J=7.6 Hz), 2.25-2.39 (m, 2H), 1.79-2.13 (m, 5H), 0.91-1.76 (br. m, 38H), 1.05 (s, 3H), 0.96 (d, 3H, J=6.4 Hz), 0.81-0.91 (m, 9H), 0.73 (s, 3H).

### AS. Compound SA83: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride

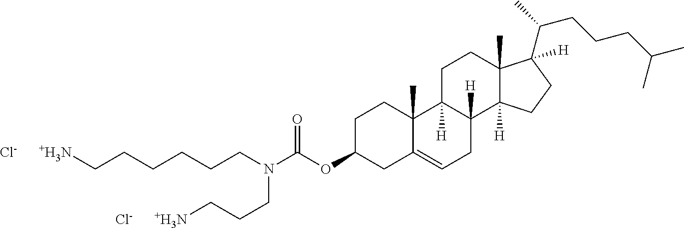

### Step 1: tert-Butyl N-[3-(2-nitrobenzenesulfonamido)propyl]carbamate

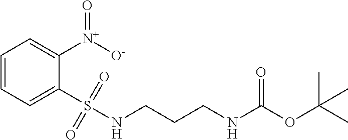 [SMILES: CC(C)(C)OC(=O)NCCCNS(=O)(=O)c1ccccc1[N+](=O)[O-]]

To a stirred solution of tert-butyl N-(3-aminopropyl)carbamate (1.50 g, 8.35 mmol) and triethylamine (1.50 mL, 10.7 mmol) in dichloromethane (40 mL) cooled to 0° C. in an ice bath, was added a solution of 2-nitrobenzenesulfonyl chloride (2.00 g, 8.75 mmol) in dichloromethane (10 mL) dropwise via addition funnel over 15 min. After this time, the reaction mixture was allowed to slowly warm to rt while stirring. The reaction was monitored by TLC. At 23 h, the reaction mixture was diluted with dichloromethane (50 mL) and then washed with 5% aq. citric acid. The organics were passed through a hydrophobic frit, dried over Na₂SO₄, and concentrated. The crude material was purified via silica gel chromatography (20-50% ethyl acetate in hexanes) to afford tert-butyl N-[3-(2-nitrobenzenesulfonamido)propyl]carbamate (2.611 g, 7.265 mmol, 87.0%) as a viscous, light yellow oil. UPLC/ELSD: RT=0.54 min. MS (ES): m/z=304.14 [(M+H) —(CH₃)₂C═CH₂]⁺ for C₁₄H₂₁N₃O₆S; ¹H NMR (300 MHz, CDCl₃): δ 8.09-8.17 (m, 1H), 7.81-7.89 (m, 1H), 7.68-7.77 (m, 2H), 5.86 (br. s, 1H), 4.66 (br. s, 1H), 3.21 (dt, 2H, J=6.2, 6.2 Hz), 3.15 (dt, 2H, J=6.4, 6.4 Hz), 1.63-1.76 (m, 2H), 1.42 (s, 9H).

### Step 2: Tert-butyl N-[3-(N-[6-[(tert-butoxycarbonyl)amino]hexyl]2-nitrobenzenesulfonamido)propyl]carbamate

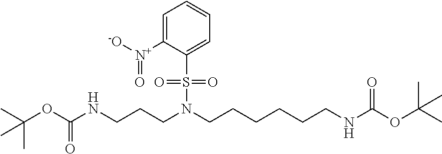

To a stirred mixture of tert-butyl N-[3-(2-nitrobenzenesulfonamido)propyl]carbamate (1.000 g, 2.782 mmol), potassium carbonate (0.769 g, 5.56 mmol), and potassium iodide (0.046 g, 0.28 mmol) in dimethylformamide (15 mL) was added a solution of tert-butyl N-(6-bromohexyl)carbamate (0.858 g, 3.06 mmol) in dimethylformamide (1.0 mL). The reaction mixture stirred at rt and was monitored by LCMS. At 20.3 h, the reaction mixture was heated to 50° C. At 25 h, the reaction mixture was cooled to rt. The reaction mixture was diluted with methyl tert-butyl ether and water. The layers were separated, and the organics were washed with water (4×) and brine, dried over Na₂SO₄, and concentrated. The crude material was purified via silica gel chromatography (10-85% methyl tert-butyl ether in hexanes) to afford tert-butyl N-[3-(N-{6-[(tert-butoxycarbonyl)amino]hexyl}2-nitrobenzenesulfonamido)propyl]carbamate (1.399 g, 2.504 mmol, 90.0%) as a clear oil. UPLC/ELSD: RT=1.43 min. MS (ES): m/z=403.26 [(M+H)−2[(CH₃)₂C═CH₂]—CO₂]⁺ for C₂₅H₄₂N₄O₈S; ¹H NMR (300 MHz, CDCl₃): δ 7.96-8.04 (m, 1H), 7.58-7.73 (m, 3H), 4.84 (br. s, 1H), 4.50 (br. s, 1H), 3.35 (t, 2H, J=7.0 Hz), 3.25 (t, 2H, J=7.6 Hz), 3.15 (td, 2H, J=6.3, 6.2 Hz), 3.06 (td, 2H, J=6.7, 6.5 Hz), 1.68-1.79 (m, 2H), 1.34-1.60 (m, 4H), 1.44 (s, 18H), 1.20-1.33 (m, 4H).

### Step 3: Tert-butyl N-[3-({6-[(tert-butoxycarbonyl)amino]hexyl}amino)propyl]carbamate

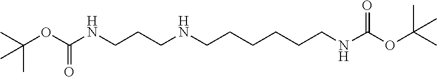 [SMILES: CC(C)(C)OC(=O)NCCCCCCNCCCNC(=O)OC(C)(C)C]

Tert-butyl N-[3-(N-{6-[(tert-butoxycarbonyl)amino]hexyl}2-nitrobenzenesulfonamido)propyl]carbamate (1.391 g, 2.490 mmol), potassium carbonate (1.032 g, 7.469 mmol), and thiophenol (0.39 mL, 3.82 mmol) were combined in dimethylformamide (20 mL). The reaction mixture stirred at rt and was monitored by LCMS. At 18 h, the reaction mixture was filtered through a pad of Celite rinsing with methyl tert-butyl ether. The filtrate was washed with saturated aq. NaHCO₃ solution, water (3×), and brine. The organics were dried over Na₂SO₄ and concentrated. The crude material was purified via silica gel chromatography (0-20% (5% conc. aq. NH₄OH in methanol) in dichloromethane) to afford tert-butyl N-[3-({6-[(tert-butoxycarbonyl)amino]hexyl}amino)propyl]carbamate (0.889 g, 2.38 mmol, 95.6%) as a white solid. UPLC/ELSD: RT=0.42 min. MS (ES): m/z=374.38 [M+H]⁺ for C₁₉H₃₉N₃O₄; ¹H NMR (300 MHz, CDCl₃): δ 5.15 (br. s, 1H), 4.52 (br. s, 1H), 3.19 (dt, 2H, J=5.9, 5.9 Hz), 3.10 (dt, 2H, J=6.4, 6.4 Hz), 2.66 (t, 2H, J=6.6 Hz), 2.57 (t, 2H, J=7.0 Hz), 1.59-1.71 (m, 2H), 1.24-1.55 (m, 9H), 1.44 (s, 18H).

### Step 4: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate

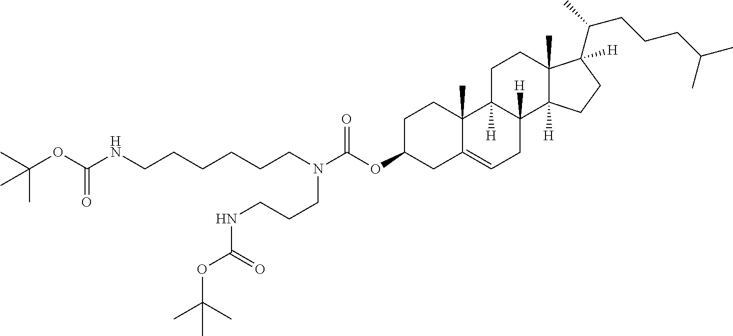

Cholesterol 4-nitrophenyl carbonate (0.200 g, 0.362 mmol), tert-butyl N-[3-({6-[(tert-butoxycarbonyl)amino]hexyl}amino)propyl]carbamate (0.169 g, 0.453 mmol), and triethylamine (0.15 mL, 1.1 mmol) were combined in toluene (3.5 mL). The reaction mixture stirred at 90° C. and was monitored by LCMS. At 17 h, the reaction mixture was cooled to rt, diluted with dichloromethane (30 mL), and washed with 5% aq. NaHCO₃ solution (3×30 mL). The organics were passed through a hydrophobic frit, dried over Na₂SO₄, and concentrated. The crude material was purified via silica gel chromatography (20-50% ethyl acetate in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate (0.240 g, 0.305 mmol, 84.2%) as a clear oil. UPLC/ELSD: RT=3.43 min. MS (ES): m/z=687.36 [(M+H)—(CH₃)₂C═CH₂—CO₂]⁺ for C₄₇H₈₃N₃O₆; ¹H NMR (300 MHz, CDCl₃): S65.17-5.44 (m, 2H), 4.40-4.86 (m, 2H), 2.98-3.40 (br. m, 8H), 2.19-2.44 (m, 2H), 1.74-2.10 (m, 5H), 0.93-1.73 (br. m, 31H), 1.44 (s, 18H), 1.02 (s, 3H), 0.91 (d, 3H, J=6.4 Hz), 0.86 (d, 3H, J=6.6 Hz), 0.86 (d, 3H, J=6.6 Hz), 0.68 (s, 3H₁).

### Step 5: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-JH-cyclopenta[a]phenanthren-3-yl (6-aminohexyl)(3-aminopropyl)carbamate dihydrochloride

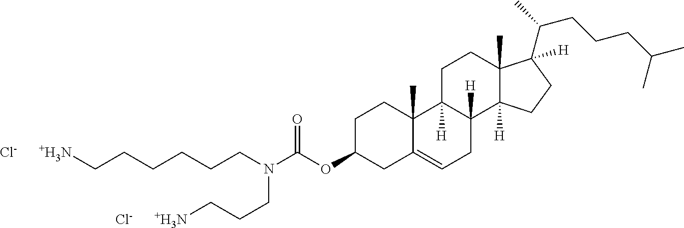

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate (0.234 g, 0.298 mmol) in isopropanol (2.8 mL) was added 5-6 N HCl in isopropanol (0.42 mL). The reaction mixture stirred at 40° C. and was monitored by LCMS. At 17 h, the reaction mixture was cooled to rt and then acetonitrile (8.4 mL) was added. The suspension stirred at rt for 1 h and then solids were collected by vacuum filtration rinsing with cold 3:1 acetonitrile/isopropanol to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride (0.177 g, 0.264 mmol, 88.8%) as a white solid. UPLC/ELSD: RT=1.98 min. MS (ES): m/z=314.63 [(M+2H)+CH₃CN]²⁺ for C₃₇H₆₇N₃O₂; ¹H NMR (300 MHz, CD₃OD): δ 5.35-5.44 (m, 1H), 4.36-4.52 (m, 1H), 3.37 (t, 2H, J=6.8 Hz), 3.28 (t, 2H, J=7.6 Hz), 2.88-2.99 (m, 4H), 2.26-2.46 (m, 2H), 1.78-2.14 (m, 7H), 0.98-1.75 (br. m, 29H), 1.06 (s, 3H), 0.95 (d, 3H, J=6.4 Hz), 0.88 (d, 3H, J=6.6 Hz), 0.88 (d, 3H, J=6.6 Hz), 0.73 (s, 3H).

### AT. Compound SA84: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride

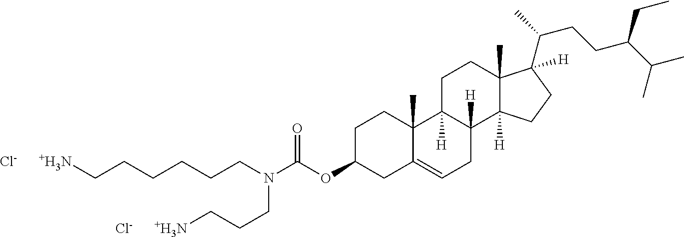

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate

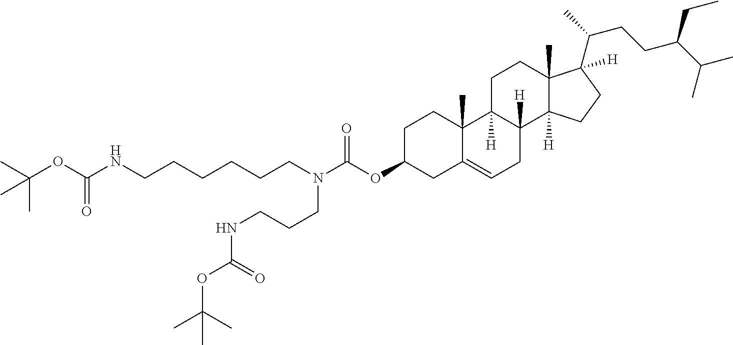

β-Sitosterol 4-nitrophenyl carbonate (0.200 g, 0.345 mmol), tert-butyl N-[3-({6-[(tert-butoxycarbonyl)amino]hexyl}amino)propyl]carbamate (0.161 g, 0.431 mmol), and triethylamine (0.15 mL, 1.1 mmol) were combined in toluene (3.5 mL). The reaction mixture stirred at 90° C. and was monitored by LCMS. At 17 h, the reaction mixture was cooled to rt, diluted with dichloromethane (30 mL), and washed with 5% aq. NaHCO₃ solution (3×30 mL). The organics were passed through a hydrophobic frit, dried over Na₂SO₄, and concentrated. The crude material was purified via silica gel chromatography (20-50% ethyl acetate in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate (0.218 g, 0.268 mmol, 77.6%) as a clear oil. UPLC/ELSD: RT=3.55 min. MS (ES): m/z=715.12 [(M+H)—(CH₃)₂C═CH₂—CO₂]⁺ for C₄₉H₈₇N₃O₆; ¹H NMR (300 MHz, CDCl₃): δ 5.13-5.44 (m, 2H), 4.35-4.88 (m, 2H), 2.98-3.40 (br. m, 8H), 2.20-2.45 (m, 2H), 1.76-2.09 (m, 5H), 0.88-1.75 (br. m, 32H), 1.44 (s, 18H), 1.02 (s, 3H), 0.92 (d, 3H, J=6.4 Hz), 0.78-0.89 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride

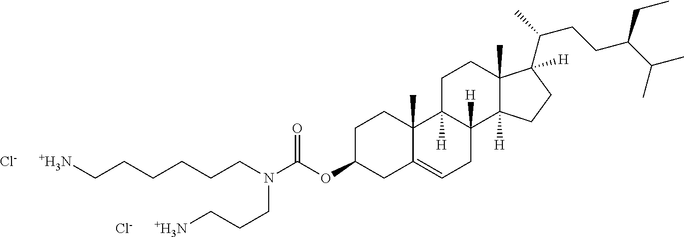

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate (0.213 g, 0.262 mmol) in isopropanol (2.6 mL) was added 5-6 N HCl in isopropanol (0.38 mL). The reaction mixture stirred at 40° C. and was monitored by LCMS. At 17 h, the reaction mixture was cooled to rt, and then acetonitrile (7.8 mL) was added. The suspension stirred at rt for 1 h, and then solids were collected by vacuum filtration rinsing with cold 3:1 acetonitrile/isopropanol to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride (0.169 g, 0.232 mmol, 88.8%) as a white solid. UPLC/ELSD: RT=2.17 min. MS (ES): m/z=328.70 [(M+2H)+CH₃CN]²⁺ for C₃₉H₇₁N₃O₂; ¹H NMR (300 MHz, CD₃OD): δ 5.36-5.45 (m, 1H), 4.36-5.53 (m, 1H), 3.37 (t, 2H, J=6.6 Hz), 3.28 (t, 2H, J=7.5 Hz), 2.88-2.98 (m, 4H), 2.26-2.43 (m, 2H), 1.80-2.12 (m, 7H), 0.91-1.77 (br. m, 30H), 1.06 (s, 3H), 0.96 (d, 3H, J=6.4 Hz), 0.82-0.91 (m, 9H), 0.73 (s, 3H).

### AU. Compound SA87: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-fluoropropyl)(4-((3-amino-2-fluoropropyl)amino)butyl)carbamate trihydrochloride

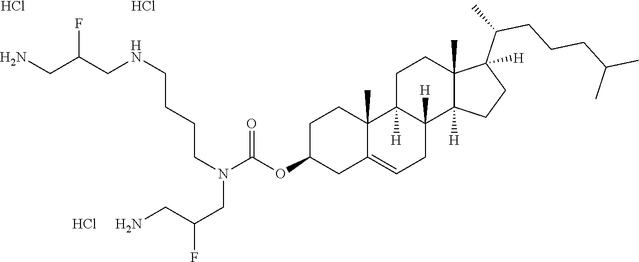

### Step 1: tert-butyl (2-fluoro-3-((2-nitrophenyl)sulfonamido)propyl)carbamate

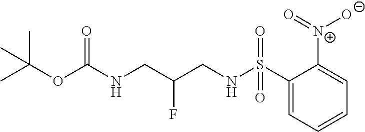 [SMILES: CC(C)(C)OC(=O)NCC(F)CNS(=O)(=O)c1ccccc1[N+](=O)[O-]]

To a solution of tert-butyl (3-amino-2-fluoropropyl)carbamate (1.00 g, 5.20 mmol) in dry DCM (15 mL) set stirring under nitrogen was added triethylamine (0.87 mL, 6.24 mmol). The solution was cooled to 0° C. and then a solution of 2-nitrobenzenesulfonyl chloride (1.27 g, 5.72 mmol) in 5 mL dry DCM was added dropwise over 30 minutes. The reaction was allowed to proceed at 0° C. for an hour and then at room temperature for an additional three hours. The mixture was then diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2-fluoro-3-((2-nitrophenyl)sulfonamido)propyl)carbamate as a white solid (1.93 g, 5.14 mmol, 98.7%). UPLC/ELSD: RT=1.76 min. MS (ES): m/z (MH⁺) 378.4 for C₁₄H₂₀FN₃O₆S. ¹H NMR (300 MHz, CDCl₃) δ: ppm 8.15 (m, 1H), 7.87 (m, 1H), 7.77 (m, 2H), 6.14 (br. s, 1H), 5.31 (m, 1H), 4.71 (m, 1H), 4.55 (m, 1H), 3.39 (m, 4H), 1.44 (s, 9H).

### Step 2: di-tert-butyl ((butane-1,4-diylbis(azanediyl))bis(2-fluoropropane-3,1-diyl))dicarbamate

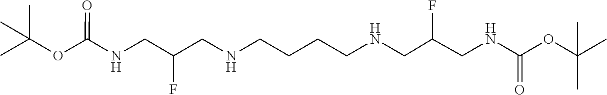 [SMILES: CC(C)(C)OC(=O)NCC(F)CNCCCCNCC(F)CNC(=O)OC(C)(C)C]

To a solution of tert-butyl (2-fluoro-3-((2-nitrophenyl)sulfonamido)propyl)carbamate (1.94 g, 5.14 mmol) in dry DMF (20 mL) set stirring under nitrogen was added potassium carbonate (2.06 g, 14.92 mmol) and 1,4-diiodobutane (0.32 mL, 2.45 mmol). The solution was heated to 40° C. and allowed to proceed overnight. The following morning, benzyl bromide (0.24 mL, 2.03 mmol) was added, and the reaction was allowed to proceed at room temperature for 8 h. Then, thiophenol (0.96 mL, 9.42 mmol), potassium carbonate (1.01 g, 7.34 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to give an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-80% (75:20:5 DCM/MeOH/concentrated aqueous ammonium hydroxide) gradient. Fractions containing product were combined and concentrated to give di-tert-butyl ((butane-1,4-diylbis(azanediyl))bis(2-fluoropropane-3,1-diyl))dicarbamate as a colorless oil (0.77 g, 1.76 mmol, 72.0%). UPLC/ELSD: RT=0.34 min. MS (ES): m/z (MH⁺) 439.6 for C₂₀H₄₀F₂N₄O₄. ¹H NMR (300 MHz, CDCl₃) δ: ppm 5.11 (m, 2H), 4.73 (br. s, 1H), 4.56 (br. s, 1H), 3.36 (m, 3H), 2.75 (br. m, 9H), 1.51 (m, 5H), 1.43 (s, 18H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-fluoropropyl)(4-((3-((tert-butoxycarbonyl)amino)-2-fluoropropyl)amino)butyl)carbamate

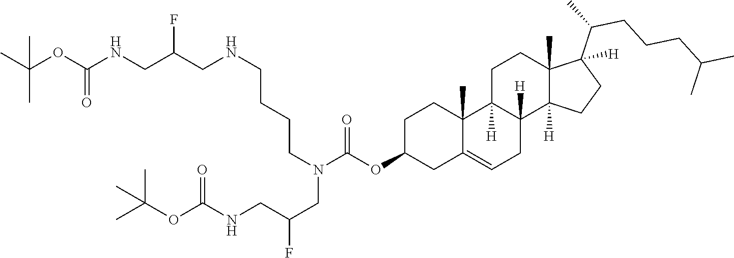

To a solution of di-tert-butyl ((butane-1,4-diylbis(azanediyl))bis(2-fluoropropane-3,1-diyl))dicarbamate (0.73 g, 1.67 mmol) in dry toluene (20 mL) set stirring under nitrogen was added triethylamine (0.59 mL, 4.18 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.77 g, 1.39 mmol) was added, and the solution was heated to 90° C. and allowed to proceed overnight. The following morning, the reaction mixture was allowed to cool to room temperature, diluted with toluene, and washed with water (3×15 mL), dried over sodium sulfate, filtered, and concentrated to give an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-fluoropropyl)(4-((3-((tert-butoxycarbonyl)amino)-2-fluoropropyl)amino)butyl)carbamate as a colorless oil (0.76 g, 0.89 mmol, 64.1%). UPLC/ELSD: RT=2.62 min. MS (ES): m/z (MH⁺) 852.3 for C₄₈H₈₄F₂N₄O₆. ¹H NMR (300 MHz, CDCl₃) δ: ppm 5.89 (m, 1H), 4.98 (br. m, 1H), 4.76 (br. m, 1H), 4.60 (m, 2H), 3.42 (br. m, 9H), 2.86 (d, 1H, J=6 Hz), 2.78 (m, 1H), 2.65 (m, 3H), 2.35 (m, 2H), 1.90 (br. m, 6H), 1.58 (br. m, 10H), 1.46 (s, 25H), 1.35 (m, 4H), 1.15 (br. m, 10H), 1.04 (s, 6H), 0.94 (d, 4H, J=6 Hz), 0.78 (d, 6H, J=6 Hz), 0.70 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-fluoropropyl)(4-((3-amino-2-fluoropropyl)amino)butyl)carbamate trihydrochloride

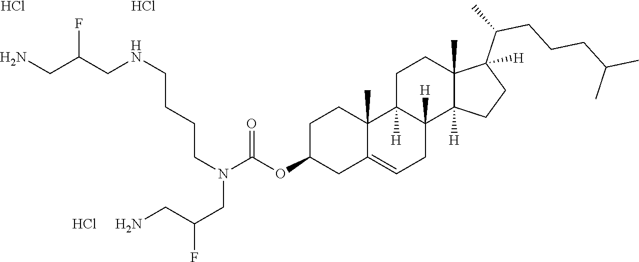

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-fluoropropyl)(4-((3-((tert-butoxycarbonyl)amino)-2-fluoropropyl)amino)butyl)carbamate (0.76 g, 0.89 mmol) in isopropanol (10 mL) set stirring under nitrogen was added hydrochloric acid (5N in isopropanol, 1.79 mL, 8.93 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-fluoropropyl)(4-((3-amino-2-fluoropropyl)amino)butyl)carbamate trihydrochloride as a white solid (0.54 g, 0.67 mmol, 75.4%). UPLC/ELSD: RT=1.65 min. MS (ES): m/z (MH⁺) 651.7 for C₃₈H₇₁Cl₃F₂N₄O₂. ¹H NMR (300 MHz, MeOD) δ: ppm 5.41 (m, 1H), 5.22 (br. m, 1H), 4.48 (br. m, 1H), 3.43 (br. m, 7H), 3.34 (s, 4H), 3.17 (m, 4H), 2.41 (d, 2H, J=3 Hz), 2.05 (br. m, 6H), 1.75 (br. m, 17H), 1.16 (br. m, 13H), 0.95 (d, 4H, J=6 Hz), 0.91 (d, 6H, J=6 Hz), 0.74 (s, 3H).

### AV. Compound SA88: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-hydroxypropyl)(4-((3-amino-2-hydroxypropyl)amino)butyl)carbamate trihydrochloride

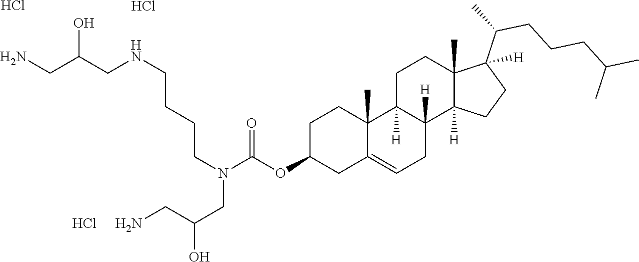

### Step 1: tert-butyl (2-hydroxy-3-((2-nitrophenyl)sulfonamido)propyl)carbamate

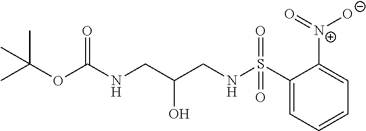 [SMILES: CC(C)(C)OC(=O)NCC(O)CNS(=O)(=O)c1ccccc1[N+](=O)[O-]]

To a solution of tert-butyl (3-amino-2-hydroxypropyl)carbamate (10.51 g, 55.27 mmol) in dry DCM (200 mL) set stirring under nitrogen was added triethylamine (9.24 mL, 66.37 mmol). The solution was cooled to 0° C., and then a solution of 2-nitrobenzenesulfonyl chloride (12.25 g, 55.27 mmol) in 100 mL dry DCM was added dropwise over 30 minutes. The reaction was allowed to proceed at 0° C. for an hour and then at room temperature for an additional three hours. The mixture was then diluted with an additional 10 mL DCM, washed with 1M aqueous sodium bicarbonate (2×100 mL), water (1×100 mL), 10% aqueous citric acid (2×100 mL), water (1×100 mL), and brine (2×100 mL), dried over sodium sulfate, filtered, and concentrated to give tert-butyl (2-hydroxy-3-((2-nitrophenyl)sulfonamido)propyl)carbamate as a white solid (17.54 g, 46.73 mmol, 84.5%). UPLC/ELSD: RT=1.23 min. MS (ES): m/z (MH⁺) 376.4 for C₁₄H₂₁N₃O₇S. ¹H NMR (300 MHz, CDCl₃) δ: ppm 8.13 (m, 1H), 7.88 (m, 1H), 7.78 (m, 2H), 6.01 (br. s, 1H), 5.01 (m, 1H), 3.86 (m, 1H), 3.29 (m, 4H), 3.12 (m, 1H), 1.45 (s, 9H).

### Step 2: di-tert-butyl ((butane-1,4-diylbis(azanediyl))bis(2-hydroxypropane-3,1-diyl))dicarbamate

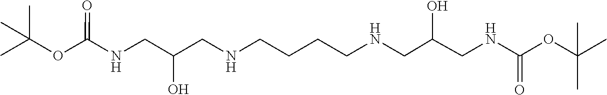 [SMILES: CC(C)(C)OC(=O)NCC(O)CNCCCCNCC(O)CNC(=O)OC(C)(C)C]

To a solution of give tert-butyl (2-hydroxy-3-((2-nitrophenyl)sulfonamido)propyl)carbamate (4.00 g, 10.66 mmol) in dry DMF (40 mL) set stirring under nitrogen was added potassium carbonate (4.28 g, 30.95 mmol) and 1,4-diiodobutane (0.67 mL, 5.07 mmol). The solution was heated to 40° C. and allowed to proceed overnight. The following morning, benzyl bromide (0.50 mL, 4.21 mmol) was added, and the reaction was allowed to proceed at room temperature for 16 h. Then, thiophenol (2.00 mL, 19.54 mmol), potassium carbonate (2.10 g, 15.22 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to give an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2-hydroxypropane-3,1-diyl))dicarbamate as a colorless oil (1.12 g, 2.58 mmol, 50.8%). UPLC/ELSD: RT=0.20 min. MS (ES): m/z (MH⁺) 435.6 for C₂₀H₄₂N₄O₆. ¹H NMR (300 MHz, CDCl₃) δ: ppm 5.47 (m, 2H), 4.72 (br. s, 1H), 3.76 (br. s, 2H), 3.47 (m, 9H), 3.22 (m, 3H), 3.05 (m, 3H), 2.62 (br. m, 8H), 1.53 (m, 5H), 1.42 (s, 18H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-hydroxypropyl)(4-((3-((tert-butoxycarbonyl)amino)-2-hydroxypropyl)amino)butyl)carbamate

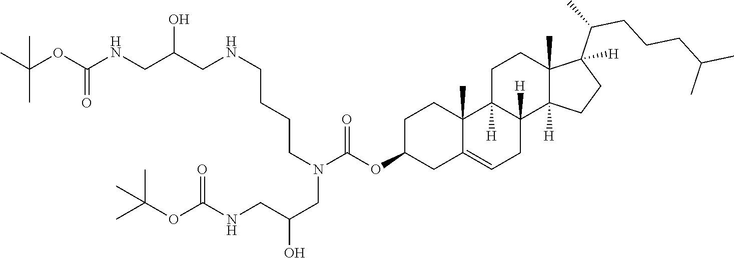

To a solution of di-tert-butyl ((butane-1,4-diylbis(azanediyl))bis(2-hydroxypropane-3,1-diyl))dicarbamate (1.12 g, 2.58 mmol) in dry toluene (20 mL) set stirring under nitrogen was added triethylamine (0.91 mL, 6.44 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (1.19 g, 2.15 mmol) was added and the solution was heated to 90° C. and allowed to proceed overnight. The following morning, the reaction mixture was allowed to cool to room temperature, diluted with toluene, washed with water (3×15 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-hydroxypropyl)(4-((3-((tert-butoxycarbonyl)amino)-2-hydroxypropyl)amino)butyl)carbamate as a colorless oil (0.08 g, 0.09 mmol, 4.1%). UPLC/ELSD: RT=2.45 min. MS (ES): m/z (MH⁺) 848.3 for C₄₈H₈₆N₄O₈. ¹H NMR (300 MHz, CDCl₃) δ: ppm 5.32 (m, 2H), 5.07 (br. m, 1H), 4.43 (br. m, 1H), 3.77 (m, 2H), 3.25 (br. m, 6H), 2.95 (m, 4H), 2.56 (m, 4H), 2.26 (m, 2H), 1.90 (m, 5H), 1.47 (m, 9H), 1.37 (s, 18H), 1.07 (m, 11H), 0.95 (s, 6H), 0.86 (d, 4H, J=6 Hz), 0.78 (d, 5H, J=6 Hz), 0.61 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl(3-amino-2-hydroxypropyl)(4-((3-amino-2-hydroxypropyl)amino)butyl)carbamate trihydrochloride

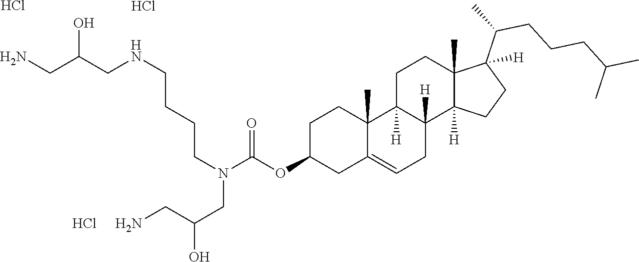

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-hydroxypropyl)(4-((3-((tert-butoxycarbonyl)amino)-2-hydroxypropyl)amino)butyl)carbamate (0.08 g, 0.09 mmol) in isopropanol (5 mL) set stirring under nitrogen was added hydrochloric acid (5N in isopropanol, 0.18 mL, 0.89 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-hydroxypropyl)(4-((3-amino-2-hydroxypropyl)amino)butyl)carbamate trihydrochloride as a white solid (0.04 g, 0.04 mmol, 49.3%). UPLC/ELSD: RT=1.53 min. MS (ES): m/z (MH⁺) 648.2 for C₃₈H₇₃Cl₃N₄O₄. ¹H NMR (300 MHz, MeOD) δ: ppm 5.42 (m, 1H), 4.45 (br. m, 1H), 4.26 (br. m, 1H), 4.08 (br. m, 1H), 3.44 (m, 3H), 3.13 (m, 9H), 2.41 (d, 2H, J=3 Hz), 2.05 (s, 3H), 1.92 (m, 3H), 1.55 (br. m, 13H), 1.16 (br. m, 11H), 0.97 (d, 3H, J=6 Hz), 0.91 (d, 5H, J=6 Hz), 0.74 (s, 3H).

### AW. Compound SA89: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride

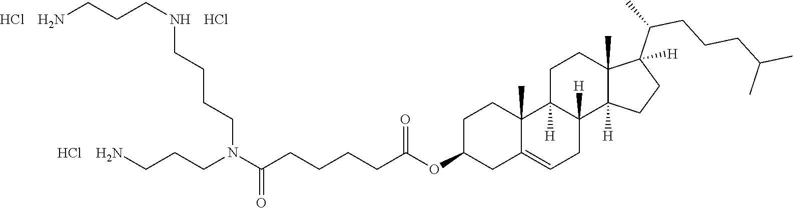

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate

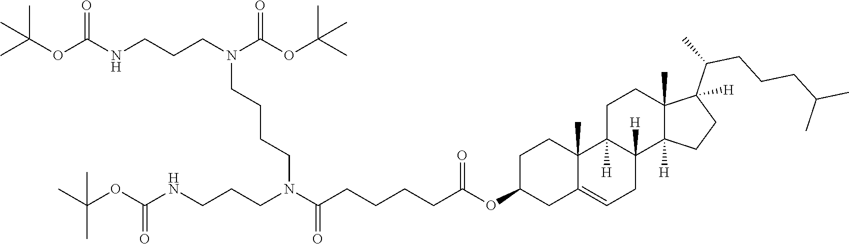

To a solution of 6-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-6-oxohexanoic acid (0.40 g, 0.77 mmol) in dry DCM (10 mL) stirring under nitrogen was added tert-butyl (3-((tert-butoxycarbonyl)amino)propyl)(4-((3-((tert-butoxycarbonyl)amino)propyl)amino)butyl)carbamate (0.43 g, 0.85 mmol), dimethylaminopyridine (0.02 g, 0.15 mmol), and 1-ethyl-3-(3-dimethylaminopropyl)carbodiimide hydrochloride (0.22 g, 1.15 mmol). The resulting solution was cooled to 0° C., and diisopropylethylamine (0.41 mL, 2.31 mmol) was added dropwise. The mixture was allowed to gradually warm to room temperature and proceed overnight. Then, the solution was diluted with dichloromethane, washed with saturated sodium bicarbonate (1×10 mL) and brine (1×10 mL), dried over sodium sulfate, filtered, and concentrated to an oil. The oil was taken up in DCM and purified on silica with a 0-80% ethyl acetate gradient in hexanes. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate as a light yellow oil (0.59 g, 0.59 mmol, 77.0%). UPLC/ELSD: RT: 3.40 min. MS (ES): m/z (MH⁺) 1000.4 for C₅₈H₁₀₂N₄O₉. ¹H NMR (300 MHz, CDCl₃) δ: ppm 5.25 (m, 1H). 4.47 (br. m, 1H), 4.00, (q, 1H), 3.12 (br. m, 12H), 2.20 (br. m, 6H), 1.91 (br. m, 8H), 1.54 (br. m, 16H), 1.31 (br. s, 33H), 1.13 (br. m, 13H), 0.90 (s, 6H), 0.81 (d, 4H, J=6 Hz), 0.73 (d, 6H, J=6 Hz), 0.56 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride

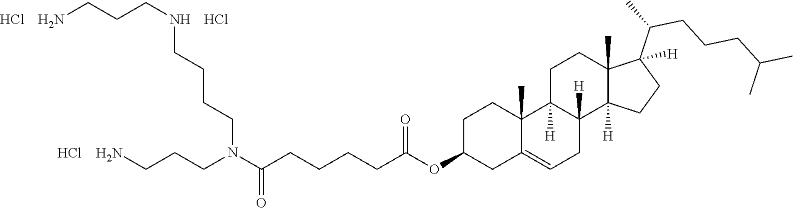

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate (0.59 g, 0.59 mmol) in isopropanol (10 mL) set stirring under nitrogen was added hydrochloric acid (5N in isopropanol, 1.19 mL, 5.92 mmol) dropwise. The solution was heated to 40° C. and allowed to proceed overnight. The following morning, dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride as a white solid (0.36 g, 0.43 mmol, 71.7%). UPLC/ELSD: RT=1.75 min. MS (ES): m/z (MH⁺) 700.1 for C₄₃H₈₁Cl₃N₄O₃. ¹H NMR (300 MHz, MeOD) δ: ppm 5.40 (m, 1H), 4.54 (br. m, 1H), 3.96 (m, 1H), 3.52 (br. m, 4H), 3.33 (s, 1H), 3.12 (m, 9H), 2.49 (br. m, 2H), 2.36 (br. m, 5H), 2.17 (m, 3H), 2.06 (s, 3H), 1.67 (br. m, 30H), 1.16 (d, 14H, J=6 Hz), 1.07 (s, 6H), 0.98 (d, 5H, J=6 Hz), 0.89 (d, 7H, J=6 Hz), 0.74 (s, 3H).

### AX. Compound SA90 (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride

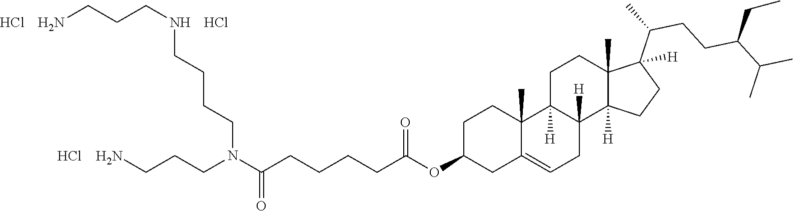

### Step 1: 6-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-6-oxohexanoic acid

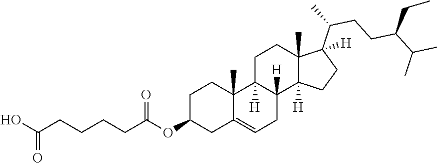

To a solution of sitosterol (0.44 g, 1.01 mmol) in dry DCM (10 mL) stirring under nitrogen was added oxepane-2,7-dione (0.13 g, 1.01 mmol), followed by dropwise addition of pyridine (0.31 mL, 2.22 mmol). The solution was then refluxed at 40° C. overnight, during which all solid went into solution. The following day, the mixture was cooled to room temperature, concentrated to a yellow oil, taken up in DCM, and purified on silica without further workup. The silica column was run in hexanes with a gradient of 0-30% EtOAc. Product-containing fractions were pooled and concentrated to give 6-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-6-oxohexanoic acid as a white solid (0.12 g, 0.21 mmol, 21.0%). UPLC/ELSD: RT: 3.23 min. MS (ES): m/z (MH⁺) 543.8 for C₃₅H₅₈O₄. ¹H NMR (300 MHz, CDCl₃) δ: ppm 5.36 (m, 1H), 3.54 (br. m, 1H), 2.28, (m, 2H), 2.04 (br. m, 3H), 1.86 (br. m, 3H), 1.49 (br. m, 19H), 1.02 (s, 6H), 0.94 (d, 5H, J=6 Hz), 0.86 (q, 10H), 0.69 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate

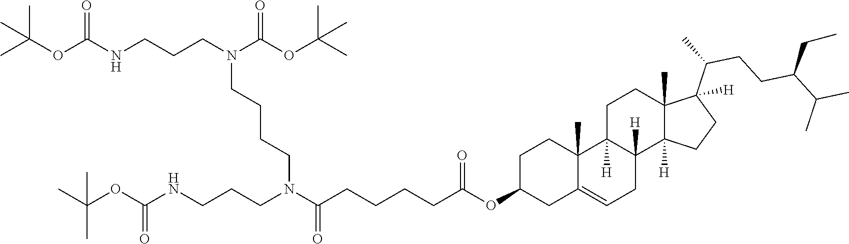

To a solution of 6-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-6-oxohexanoic acid (0.12 g, 0.21 mmol) in dry DCM (10 mL) stirring under nitrogen was added tert-butyl (3-((tert-butoxycarbonyl)amino)propyl)(4-((3-((tert-butoxycarbonyl)amino)propyl)amino)butyl)carbamate (0.13 g, 0.25 mmol), dimethylaminopyridine (0.01 g, 0.04 mmol), and 1-ethyl-3-(3-dimethylaminopropyl)carbodiimide hydrochloride (0.08 g, 0.42 mmol). The resulting solution was cooled to 0° C. and diisopropylethylamine (0.11 mL, 0.64 mmol) was added dropwise. The mixture was allowed to gradually warm to room temperature and proceed overnight. Then, the solution was diluted with dichloromethane, washed with saturated aqueous sodium bicarbonate (1×10 mL) and brine (1×10 mL), dried over sodium sulfate, filtered, and concentrated to give an oil. The oil was taken up in DCM and purified on silica with a 0-80% ethyl acetate gradient in hexanes to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate as a light yellow oil (0.15 g, 0.14 mmol, 67.5%). UPLC/ELSD: RT: 3.91 min. MS (ES): m/z (MH⁺) 1028.5 for C₆₀H₁₀₆N₄O₉. ¹H NMR (300 MHz, CDCl₃) δ: ppm 5.38 (m, 2H), 4.59 (br. m, 2H), 4.14 (m, 1H), 3.24 (br. m, 11H), 2.32 (br. m, 6H), 1.67 (br. m, 17H), 1.47 (s, 32H), 1.25 (br. m, 11H), 1.03 (s, 6H), 0.94 (d, 4H, J=9 Hz), 0.86 (q, 8H, J=9 Hz), 0.69 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride

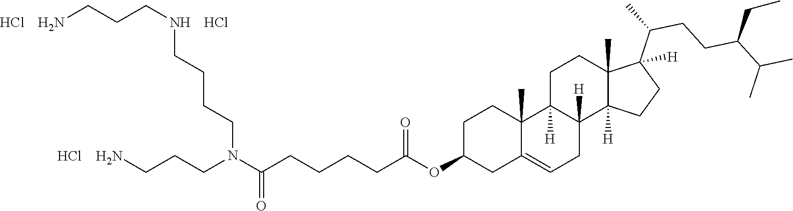

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate (0.15 g, 0.14 mmol) in isopropanol (5 mL) set stirring under nitrogen was added hydrochloric acid (5N in isopropanol, 0.29 mL, 1.43 mmol) dropwise. The solution was heated to 40° C. and allowed to proceed overnight. The following morning, dry acetonitrile (10 mL) was added to the mixture, which was sonicated and allowed to stir for an additional hour. The white solid was then filtered out of the solution, washed repeatedly with acetonitrile, and dried in vacuo to give ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride as a white solid (0.06 g, 0.06 mmol, 43.9%). UPLC/ELSD: RT=1.97 min. MS (ES): m/z (MH) 728.1 for C₄₅HSsCl₃N₄O₃. ¹H NMR (300 MHz, MeOD) δ: ppm 5.41 (m, 1H), 4.87 (br. m, 9H), 4.55 (br. m, 1H), 3.46 (m, 3H), 3.33 (s, 1H), 3.10 (m, 6H), 2.35 (br. m, 6H), 2.04 (s, 5H), 1.68 (br. m, 15H), 1.21 (m, 9H), 1.06 (s, 4H), 0.97 (d, 4H, J=6 Hz), 0.86 (q, 7H, J=6 Hz), 0.74 (s, 3H).

### AY. Compound SA95: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride

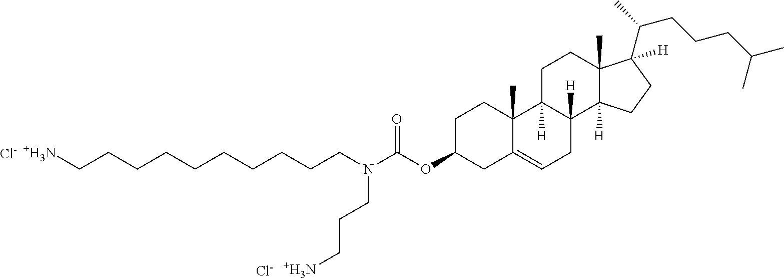

### Step 1: tert-Butyl N-{10-[(2-cyanoethyl)amino]decyl}carbamate

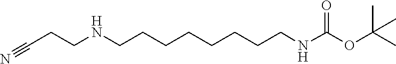 [SMILES: CC(C)(C)OC(=O)NCCCCCCCCNCCC#N]

To a suspension of tert-butyl N-(10-aminodecyl)carbamate (1.500 g, 5.506 mmol), water (15 mL), and glyme (15 mL) was added triton B (cat.). The suspension was stirred at 50° C., and then acrylonitrile (0.40 mL, 6.1 mmol) was added. The reaction mixture stirred at 50° C. and was monitored by LCMS. At 16 h, the reaction mixture was cooled to rt. The reaction mixture was concentrated and diluted with dichloromethane (100 mL) and 5% aq. NaHCO₃ solution (100 mL). The aqueous layer was extracted with dichloromethane (3×30 mL). The combined organics were passed through a hydrophobic frit, dried over Na₂SO₄, and concentrated. The crude material was purified via silica gel chromatography (0-10% methanol in dichloromethane) to afford tert-butyl N-{10-[(2-cyanoethyl)amino]decyl}carbamate (1.328 g, 3.150 mmol, 57.2%) as a white solid. UPLC/ELSD: RT=0.53 min. MS (ES): m/z=326.48 [M+H]⁺ for C₁₈H₃₅N₃O₂; ¹H NMR (300 MHz, CDCl₃): δ 4.49 (br. s, 1H), 3.09 (dt, 2H, J=6.5, 6.3 Hz), 2.93 (t, 2H, J=6.6 Hz), 2.62 (t, 2H, J=7.1 Hz), 2.52 (t, 2H, J=6.6 Hz), 1.22-1.54 (m, 17H), 1.44 (s, 9H).

### Step 2: tert-Butyl N-{10-[benzyl(2-cyanoethyl)amino]decyl}carbamate

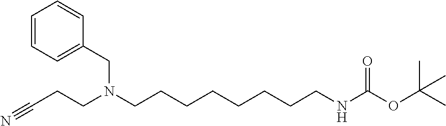 [SMILES: CC(C)(C)OC(=O)NCCCCCCCCN(CCC#N)Cc1ccccc1]

To a stirred suspension of tert-butyl N-{10-[(2-cyanoethyl)amino]decyl}carbamate (1.209 g, 2.867 mmol) and potassium carbonate (0.793 g, 5.74 mmol) in acetonitrile (18 mL) was added benzyl bromide (0.43 mL, 3.6 mmol). The reaction mixture stirred at 70° C. and was monitored by LCMS. At 16 h, the reaction mixture was cooled to rt and then filtered through a pad of Celite rinsing with acetonitrile. The filtrate was concentrated and then purified via silica gel chromatography (0-50% ethyl acetate in hexanes) to afford tert-butyl N-{10-[benzyl(2-cyanoethyl)amino]decyl}carbamate (1.283 g, quant.) as a clear oil. UPLC/ELSD: RT=0.74 min. MS (ES): m/z=416.47 [M+H]⁺ for C₂₅H₄₁N₃O₂; ¹H NMR (300 MHz, CDCl₃): δ 7.20-7.42 (m, 5H), 4.48 (br. s, 1H), 3.61 (s, 2H), 3.10 (td, 2H, J=6.6, 6.3 Hz), 2.78 (t, 2H, J=7.0 Hz), 2.48 (t, 2H, J=7.2 Hz), 2.39 (t, 2H, J=7.0 Hz), 1.39-1.58 (m, 4H), 1.44 (s, 9H), 1.21-1.35 (m, 12H).

### Step 3: Tert-butyl N-{3-[benzyl({10-[(tert-butoxycarbonyl)amino]decyl})amino]propyl}carbamate

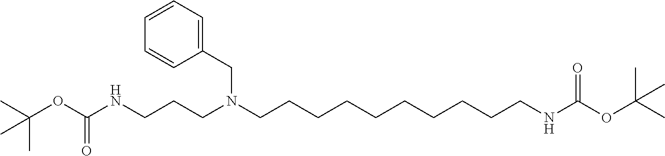 [SMILES: CC(C)(C)OC(=O)NCCCCCCCCCCN(CCCNC(=O)OC(C)(C)C)Cc1ccccc1]

Tert-butyl N-{10-[benzyl(2-cyanoethyl)amino]decyl}carbamate (0.050 g, 0.12 mmol), di-tert-butyl dicarbonate (0.053 g, 0.24 mmol), and nickel (II) chloride (0.016 g, 0.12 mmol) were combined in ethanol (1.0 mL). The stirred reaction mixture was cooled in an ice bath to 0° C., and then sodium borohydride (0.014 g, 0.36 mmol) was added. The reaction mixture was allowed to come to rt and was monitored by LCMS. At 21 h, diethylenetriamine (0.03 mL, 0.3 mmol) was added. The reaction mixture stirred at rt for 2 h and then filtered through a pad of Celite. The filtrate was concentrated, suspended in a 5% aq. NaHCO₃ solution (25 mL), and extracted with ethyl acetate (3×15 mL). The combined organics were washed with brine, dried over Na₂SO₄, and concentrated. The crude material was purified via silica gel chromatography (0-7% methanol in dichloromethane) to afford tert-butyl N-{3-[benzyl({10-[(tert-butoxycarbonyl)amino]decyl})amino]propyl}carbamate (0.040 g, 0.077 mmol, 64.0%) as a clear oil. UPLC/ELSD: RT=1.25 min. MS (ES): m/z=520.77 [M+H]⁺ for C₃₀H₅₃N₃O₄; ¹H NMR (300 MHz, CDCl₃): δ 7.19-7.39 (m, 5H), 5.52 (br. s, 1H), 4.49 (br. s, 1H), 3.51 (s, 2H), 3.01-3.22 (m, 4H), 2.46 (t, 2H, J=6.1 Hz), 2.37 (t, 2H, J=7.1 Hz), 1.37-1.68 (m, 6H), 1.44 (s, 9H), 1.44 (s, 9H), 1.15-1.35 (m, 12H).

### Step 4: Tert-butyl N-[3-({10-[(tert-butoxycarbonyl)amino]decyl}amino)propyl]carbamate

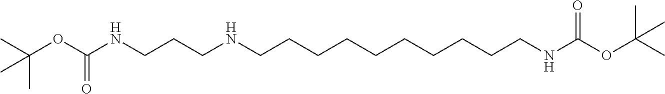 [SMILES: CC(C)(C)OC(=O)NCCCCCCCCCCNCCCNC(=O)OC(C)(C)C]

Tert-butyl N-{3-[benzyl({10-[(tert-butoxycarbonyl)amino]decyl})amino]propyl}carbamate (0.340 g, 0.654 mmol) and 10% Pd/C (0.139 g, 0.065 mmol) were combined in ethanol (5.1 mL) and then stirred under a balloon of H₂ at rt. The reaction was monitored by LCMS. At 18 h the reaction mixture was diluted with ethyl acetate (10 mL) and then was filtered through a pad of Celite rinsing with ethyl acetate. The filtrate was concentrated, taken up in ethyl acetate, filtered through a 0.45 μm PTFE syringe filter, and concentrated to afford tert-butyl N-[3-({10-[(tert-butoxycarbonyl)amino]decyl}amino)propyl]carbamate (0.238 g, 0.554 mmol, 84.7%) as a white solid. UPLC/ELSD: RT=0.97 min. MS (ES): m/z=430.42 [M+H]⁺ for C₂₃H₄₇N₃O₄; ¹H NMR (300 MHz, CDCl₃): δ 5.17 (br. s, 11H), 4.49 (br. s, 1H), 3.20 (dt, 2H, J=6.1, 6.0 Hz), 3.09 (dt, 2H, J=6.6, 6.4 Hz), 2.67 (t, 2H, J=6.6 Hz), 2.58 (t, 2H, J=7.1 Hz), 1.17-1.76 (br. m, 19H), 1.44 (s, 18H).

### Step 5: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate

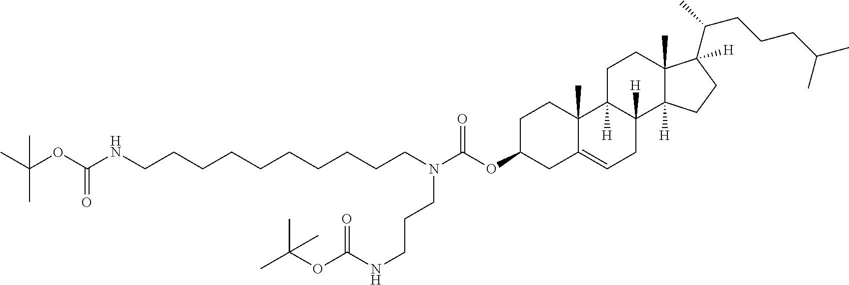

Cholesterol 4-nitrophenyl carbonate (0.112 g, 0.203 mmol), tert-butyl N-[3-({10-[(tert-butoxycarbonyl)amino]decyl}amino)propyl]carbamate (0.103 g, 0.240 mmol), and triethylamine (0.088 mL, 0.63 mmol) were combined in toluene (3.0 mL). The reaction mixture stirred at 90° C. and was monitored by LCMS. At 17 h the reaction mixture was heated at 100° C. At 20 h DMAP (cat.) was added. At 41 h the reaction mixture was cooled to rt, diluted with dichloromethane (20 mL), and then washed with 5% aq. NaHCO₃ solution (3×). The organics were passed through a hydrophobic frit, dried over Na₂SO₄, and concentrated. The crude material was purified via silica gel chromatography (10-30% ethyl acetate in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate (0.157 g, 0.186 mmol, 91.9%) as a clear oil. UPLC/ELSD: RT=3.72 min. MS (ES): m/z=743.62 [(M+H)—(CH₃)₂C═CH₂—CO₂]⁺ for C₅₁H₉₁N₃O₆; ¹H NMR (300 MHz, CDCl₃): δ 5.20-5.43 (m, 2H), 4.40-4.83 (m, 2H), 3.02-3.37 (m, 8H), 2.21-2.43 (m, 2H), 1.75-2.10 (m, 5H), 0.93-1.75 (br. m, 39H), 1.44 (s, 9H), 1.44 (s, 9H), 1.02 (s, 3H), 0.92 (d, 3H, J=6.4 Hz), 0.87 (d, 3H, J=6.5 Hz), 0.86 (d, 3H, J=6.5 Hz), 0.68 (s, 3H).

### Step 6: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride

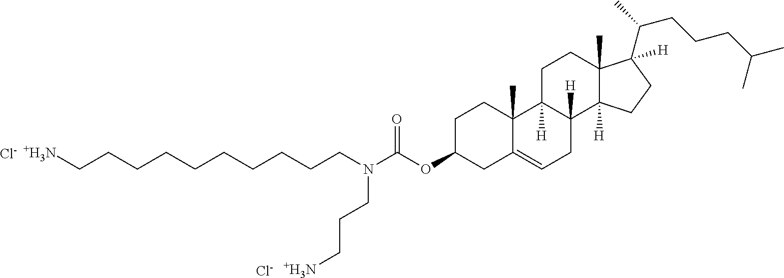

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate (0.154 g, 0.183 mmol) in isopropanol (2.5 mL) was added 5-6 N HCl in isopropanol (0.28 mL). The reaction mixture stirred at 40° C. and was monitored by LCMS. At 17 h the reaction mixture was cooled to rt and then acetonitrile (7.5 mL) was added. Solids were collected by vacuum filtration rinsing with cold 3:1 acetonitrile/isopropanol to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride (0.080 g, 0.11 mmol, 59.2%) as a white solid. UPLC/ELSD: RT=2.23 min. MS (ES): m/z=342.41 [(M+2H)+CH₃CN]²⁺ for C₄₁H₇₅N₃O₂; ¹H NMR (300 MHz, CD₃OD): δ 5.35-5.45 (m, 1H), 4.36-4.51 (m, 1H), 3.36 (t, 2H, J=6.9 Hz), 3.26 (t, 2H, J=7.4 Hz), 2.87-2.98 (m, 4H), 2.27-2.43 (m, 2H), 1.78-2.12 (m, 7H), 0.97-1.73 (br. m, 37H), 1.06 (s, 3H), 0.95 (d, 3H, J=6.4 Hz), 0.88 (d, 3H, J=6.6 Hz), 0.88 (d, 3H, J=6.6 Hz), 0.73 (s, 3H).

### AZ. Compound SA96: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride

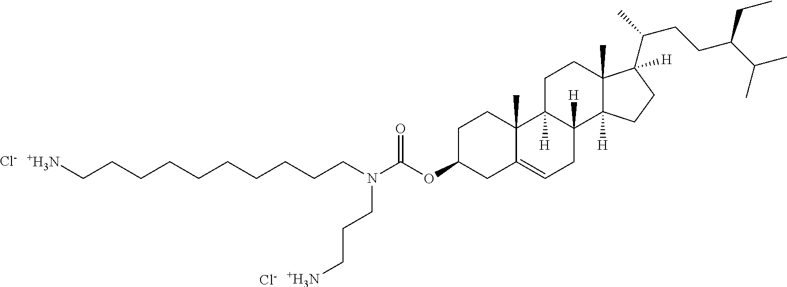

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate

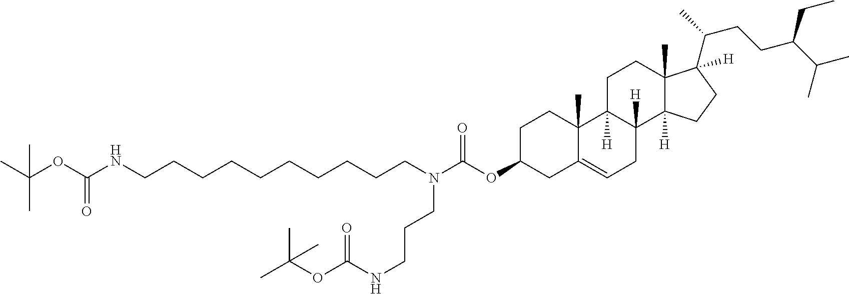

β-Sitosterol 4-nitrophenyl carbonate (0.140 g, 0.241 mmol), tert-butyl N-[3-({10-[(tert-butoxycarbonyl)amino]decyl}amino)propyl]carbamate (0.119 g, 0.277 mmol), and triethylamine (0.10 mL, 0.75 mmol) were combined in toluene (3.5 mL). The reaction mixture stirred at 90° C. and was monitored by LCMS. At 17 h the reaction mixture was heated to 100° C. At 20 h DMAP (cat.) was added. At 41 h the reaction mixture was cooled to rt, diluted with dichloromethane (20 mL), and then washed with 5% aq. NaHCO₃ solution (3×). The organics were passed through a hydrophobic frit, dried over Na₂SO₄, and concentrated. The crude material was purified via silica gel chromatography (10-30% ethyl acetate in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate (0.187 g, 0.215 mmol, 89.0%) as a clear oil. UPLC/ELSD: RT=3.80 min. MS (ES): m/z=770.03 [(M+H)—(CH₃)₂C═CH₂— CO₂]⁺ for C₅₃H₉₅N₃O₆; ¹H NMR (300 MHz, CDCl₃): δ 5.18-5.43 (m, 2H), 4.40-4.83 (m, 2H), 2.97-3.41 (m, 8H), 2.21-2.44 (m, 2H), 1.76-2.13 (m, 5H), 0.87-1.74 (br. m, 40H), 1.44 (s, 9H), 1.44 (s, 9H), 1.02 (s, 3H), 0.92 (d, 3H, J=6.4 Hz), 0.78-0.87 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-JH-cyclopenta[a]phenanthren-3-yl (10-aminodecyl)(3-aminopropyl)carbamate dihydrochloride

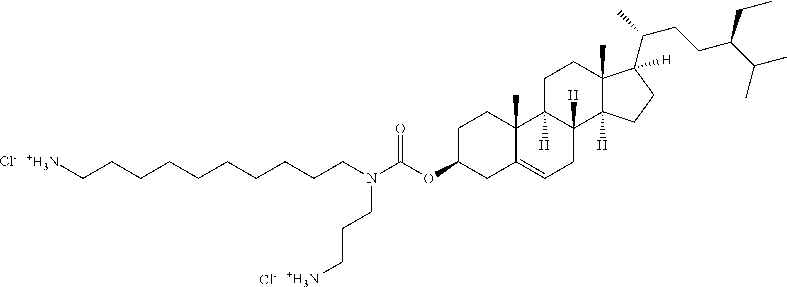

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate (0.182 g, 0.209 mmol) in isopropanol (2.5 mL) was added 5-6 N HCl in isopropanol (0.28 mL). The reaction mixture stirred at 40° C. and was monitored by LCMS. At 17 h the reaction mixture was cooled to rt and then acetonitrile (8.25 mL) was added. The mixture was concentrated. Methyl tert-butyl ether (ca. 10 mL) was added. The mixture was concentrated. The residue was dissolved in isopropanol (1.5 mL) and then added dropwise to acetonitrile (10 mL). The mixture was concentrated. Acetonitrile/methyl tert-butyl ether/isopropanol (85:10:5, ca. 10 mL) was added. The mixture was concentrated. The residue was dissolved in isopropanol (1.5 mL) and then added dropwise to 3:1 hexanes/ethyl acetate (10 mL). The mixture was concentrated. The residue was dissolved in isopropanol (1.5 mL) and then 9:1 acetonitrile/ethanol (10 mL) was added. Then acetonitrile (10 mL) was added. The supernatant was decanted, and solids were dried under vacuum to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride (0.075 g, 0.089 mmol, 42.7%) as a white solid. UPLC/ELSD: RT=2.34 min. MS (ES): m/z=356.73 [(M+2H)+CH₃CN]²⁺ for C₄₃H₇₉N₃O₂; ¹H NMR (300 MHz, CD₃OD): δ 5.36-5.46 (m, 1H), 4.36-4.52 (m, 1H), 3.36 (t, 2H, J=6.8 Hz), 3.26 (t, 2H, J=7.3 Hz), 2.87-2.98 (m, 4H), 2.27-2.44 (m, 2H), 1.80-2.12 (m, 7H), 0.91-1.77 (br. m, 38H), 1.06 (s, 3H), 0.96 (d, 3H, J=6.4 Hz), 0.79-0.91 (m, 9H), 0.73 (s, 3H).

### BA. Compound SA97: N-(8-Aminooctyl)-N-(3-aminopropyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

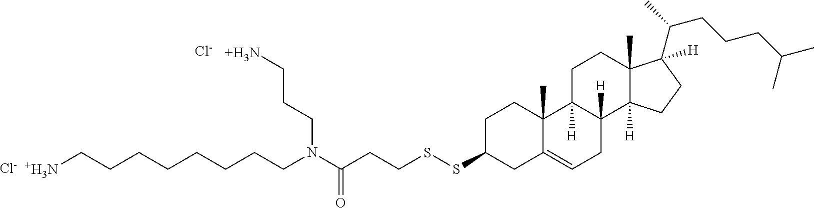

### Step 1: 2-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pyridine

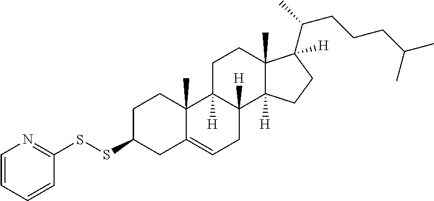

Thiocholesterol (2.000 g, 4.966 mmol) and 2,2′-dipyridyldisulfide (1.204 g, 5.463 mmol) were combined in chloroform (12.5 mL). The reaction mixture stirred at rt and was monitored by LCMS. At 20 h the reaction mixture was concentrated, and then methanol (35 mL) was added. The resulting mixture was let stand for 2 h. After this time, solids were ground with methanol as a slurry by mortar and pestle, and then solids were collected by vacuum filtration to afford 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pyridine (1.812 g, 3.540 mmol, 71.3%) as a light tan solid. UPLC/ELSD: RT=3.45 min. MS (ES): m/z=512.62 [M+H]⁺ for C₃₂H₄₉NS₂; ¹H NMR (300 MHz, CDCl₃): δ 8.39-8.49 (m, 1H), 7.72-7.83 (m, 1H), 7.57-7.69 (m, 1H), 7.01-7.12 (m, 1H), 5.27-5.43 (m, 1H), 2.70-2.88 (m, 1H), 2.20-2.47 (m, 2H), 0.78-2.11 (br. m, 38H), 0.66 (s, 3H).

### Step 2: 2-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate

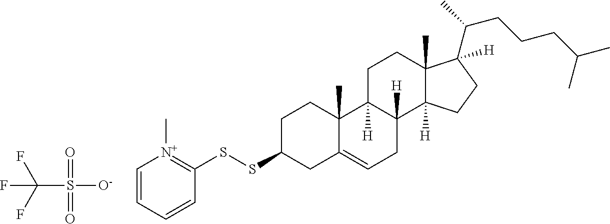

To a solution of 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pyridine (1.807 g, 3.530 mmol) in dichloromethane (3.5 ml) and heptanes (35 mL) was added methyl trifluoromethanesulfonate (0.40 mL, 3.5 mmol) dropwise over 10 min. The reaction mixture stirred at rt and was monitored by TLC. At 4 h additional trifluoromethanesulfonate (0.08 mL) was added dropwise. At 4 h 40 min solids were collected via vacuum filtration rinsing with heptanes to afford 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (2.011 g, 2.975 mmol, 84.3%) as an off-white solid. ¹H NMR (300 MHz, CD₃CN): δ 8.51-8.63 (m, 2H), 8.30-8.41 (m, 1H), 7.66-7.75 (m, 1H), 5.34-5.45 (m, 1H), 4.19 (s, 3H), 2.87-3.06 (m, 1H), 2.33-2.49 (m, 2H), 0.78-2.08 (br. m, 38H), 0.69 (s, 3H).

### Step 3: 3-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid

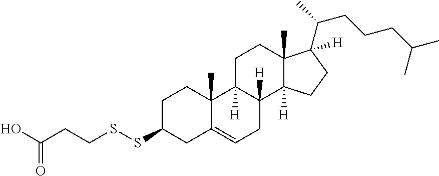

To a mixture of 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (1.000 g, 1.479 mmol) in dimethylformamide (6.5 mL) was added 3-mercaptopropionic acid (0.14 mL, 1.6 mmol). The reaction mixture stirred at rt and was monitored by LCMS. At 21 h additional 3-mercaptopropionic acid (0.02 mL) was added. At 24 h water (8 mL) was added, and the reaction mixture stirred at rt for 30 min and then was sonicated. Solids were collected by vacuum filtration rinsing with water. The wet solids were then dissolved in dichloromethane and passed through a hydrophobic frit, dried over Na₂SO₄, and concentrated. Acetonitrile (40 mL) was added to the residue, which was then sonicated. Solids were collected by vacuum filtration to afford 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.541 g, 1.07 mmol, 72.2%) as a white solid. UPLC/ELSD: RT=3.26 min.; ¹H NMR (300 MHz, CDCl₃): δ 10.32 (br. s, 1H), 5.31-5.40 (m, 1H), 2.86-2.95 (m, 2H), 2.75-2.84 (m, 2H), 2.59-2.73 (m, 1H), 2.23-2.41 (m, 2H), 1.74-2.08 (m, 5H), 0.93-1.70 (br. m, 21H), 1.00 (s, 3H), 0.92 (d, 3H, J=6.5 Hz), 0.87 (d, 3H, J=6.6 Hz), 0.86 (d, 3H, J=6.6 Hz), 0.68 (s, 3H).

### Step 4: tert-Butyl (3-(N-(8-((tert-butoxycarbonyl)amino)octyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)propyl)carbamate

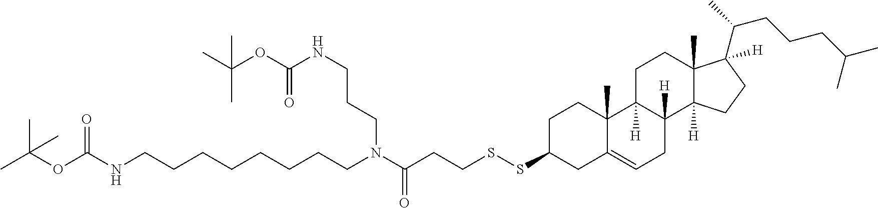

To a mixture of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.200 g, 0.395 mmol), tert-butyl N-[3-({8-[(tert-butoxycarbonyl)amino]octyl}amino)propyl]carbamate (0.222 g, 0.552 mmol), and N-hydroxysuccinimide (0.068 g, 0.59 mmol) in dichloromethane (6.0 mL) was added dicyclohexylcarbodiimide (0.138 g, 0.671 mmol). The reaction mixture stirred at rt and was monitored by LCMS. At 17 h, additional N-hydroxysuccinimide (15 mg) and dicyclohexylcarbodiimide (35 mg) were added. At 5 days the reaction mixture was filtered through a pad of Celite rinsing with dichloromethane. The filtrate was concentrated, taken up in 9:1 hexanes/ethyl acetate (10 mL), filtered, and concentrated. The crude material was purified via silica gel chromatography (10-50% ethyl acetate in hexanes) to afford tert-butyl (3-(N-(8-((tert-butoxycarbonyl)amino)octyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)propyl)carbamate (0.203 g, 0.228 mmol, 57.8%) as a clear oil. UPLC/ELSD: RT=3.61 min. MS (ES): m/z=790.32 [(M+H)—(CH₃)₂C═CH₂— CO₂]⁺ for C₅₁H₉₁N₃O₅S₂; ¹H NMR (300 MHz, CDCl₃): δ 5.24-5.42 (m, 2H), 4.42-4.67 (m, 1H), 2.91-3.48 (br. m, 10H), 2.57-2.78 (m, 3H), 2.27-2.38 (m, 2H), 0.93-2.09 (br. m, 40H), 1.44 (s, 9H), 1.43 (s, 9H), 1.00 (s, 3H), 0.91 (d, 3H, J=6.4 Hz), 0.87 (d, 3H, J=6.5 Hz), 0.86 (d, 3H, J=6.6 Hz), 0.68 (s, 3H).

### Step 5: N-(8-Aminooctyl)-N-(3-aminopropyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

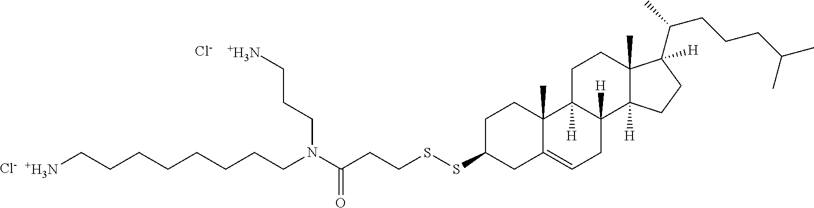

To a mixture of tert-butyl (3-(N-(8-((tert-butoxycarbonyl)amino)octyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)propyl)carbamate (0.200 g, 0.225 mmol) in isopropanol (3.0 mL) was added 5-6 N HCl in isopropanol (0.32 mL). The reaction mixture stirred at 40° C. and was monitored by LCMS. At 17 h the reaction mixture was cooled to rt, and then acetonitrile (9 mL) was added. The material was concentrated and then taken up in 4:1 acetonitrile/methanol (10 mL). The suspension was filtered rinsing with methanol. The filtrate was concentrated, triturated with 19:1 acetonitrile/ethanol (10 mL), dissolved in methanol, and concentrated to afford N-(8-aminooctyl)-N-(3-aminopropyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride (0.126 g, 0.154 mmol, 68.4%) as a white solid. UPLC/ELSD: RT=2.28 min. MS (ES): m/z=366.60 [(M+2H)+CH₃CN]²⁺ for C₄₁H₇₅N₃OS₂.

### BB. Compound SA98 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((3-aminopropyl)(4-((3-aminopropyl)amino)butyl)amino)-2-oxoethyl)disulfaneyl)acetate trihydrochloride

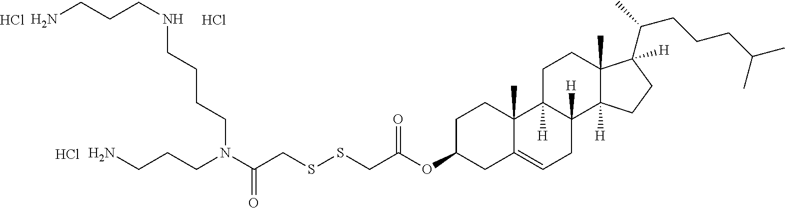

### Step 1: 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid

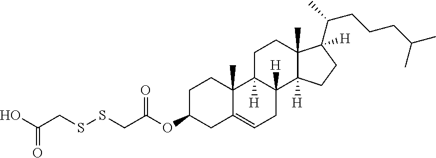

To a solution of cholesterol (5.00 g, 12.93 mmol) in dry DCM (100 mL) stirring under nitrogen was added dithiodiglycolic acid (4.53 mL, 25.86 mmol). The solution was then cooled to 0° C. and dimethylaminopyridine (0.32 g, 2.59 mmol) and 1-ethyl-3-(3-dimethylaminopropyl)carbodiimide hydrochloride (4.96 g, 25.86 mmol) were added, followed by dropwise addition of triethylamine (4.52 mL, 25.86 mmol). The solution was allowed to gradually warm to room temperature and stir overnight. The following day, the solution was washed with saturated sodium bicarbonate (1×25 mL) and water (1×25 mL), dried over sodium sulfate, filtered, and concentrated to a brown oil. The oil was taken up in DCM and purified on silica in hexanes with a 0-100% EtOAc gradient. Product-containing fractions were pooled and concentrated to give 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid as a dark brown solid (3.76 g, 6.82 mmol, 52.7%). UPLC/ELSD: RT: 3.11 min. MS (ES): m/z (MH⁺) 551.8 for C₃₁H₅₀O₄S₂. ¹H NMR (300 MHz, CDCl₃) δ: ppm 9.04 (br. s, 1H), 5.41 (m, 1H), 4.69 (br. m, 1H), 3.65 (s, 2H), 3.60 (s, 1H), 2.39 (d, 2H, J=9 Hz), 2.01 (br. m, 5H), 1.52 (br. m, 11H), 1.16 (br. m, 6H), 1.04 (s, 6H), 0.95 (d, 3H, J=6 Hz), 0.86 (d, 6H, J=6 Hz), 0.70 (s, 3H).

### Step 2: (3S,8S,9S,10R,13R,14S,17R)-10,13-dimethyl-17-((R)-6-methylheptan-2-yl)-2,3,4, 7, 8,9,10,11,12,13,14,15,16,17-tetradecahydro-JH-cyclopenta[a]phenanthren-3-yl 12-(tert-butoxycarbonyl)-7-(3-((tert-butoxycarbonyl)amino)propyl)-19,19-dimethyl-6,17-dioxo-18-oxa-3,4-dithia-7,12,16-triazaicosanoate

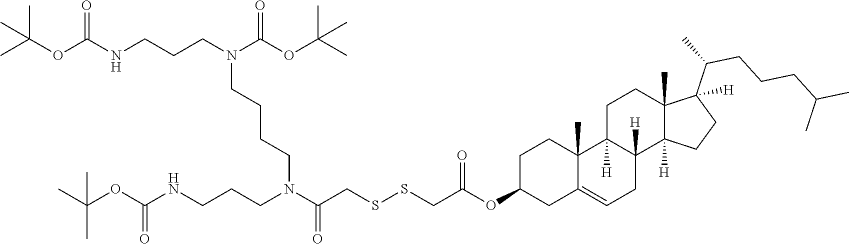

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.30 g, 0.55 mmol) in dry DCM (5 mL) stirring under nitrogen was added tert-butyl (3-((tert-butoxycarbonyl)amino)propyl)(4-((3-((tert-butoxycarbonyl)amino)propyl)amino)butyl)carbamate (0.41 g, 0.82 mmol), dimethylaminopyridine (0.03 g, 0.27 mmol), and 1-ethyl-3-(3-dimethylaminopropyl)carbodiimide hydrochloride (0.26 g, 1.36 mmol). The reaction was allowed to proceed at room temperature overnight. Then, the solution was diluted with dichloromethane, washed with saturated aqueous sodium bicarbonate (1×10 mL) and brine (1×10 mL), dried over sodium sulfate, filtered, and concentrated to an oil. The oil was taken up in DCM and purified on silica with a 0-80% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 12-(tert-butoxycarbonyl)-7-(3-((tert-butoxycarbonyl)amino)propyl)-19,19-dimethyl-6,17-dioxo-18-oxa-3,4-dithia-7,12,16-triazaicosanoate as a light yellow oil (0.33 g, 0.31 mmol, 57.6%). UPLC/ELSD: RT: 3.46 min. MS (ES): m/z (MH) 1036.5 for C₅₆H₉₈N₄O₉S2. ¹H NMR (300 MHz, CDCl₃) δ: ppm 5.33 (m, 2H), 4.61 (br. m, 1H), 3.72 (s, 2H), 3.51 (s, 2H), 3.29 (br. m, 11H), 2.28 (d, 2H, J=6 Hz), 1.81 (br. m, 6H), 1.50 (s, 26H), 1.20 (br. m, 11H), 0.97 (s, 5H), 0.88 (d, 3H, J=6 Hz), 0.82 (d, 5H, J=6 Hz), 0.63 (s, 3H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((3-aminopropyl)(4-((3-aminopropyl)amino)butyl)amino)-2-oxoethyl)disulfaneyl)acetate trihydrochloride

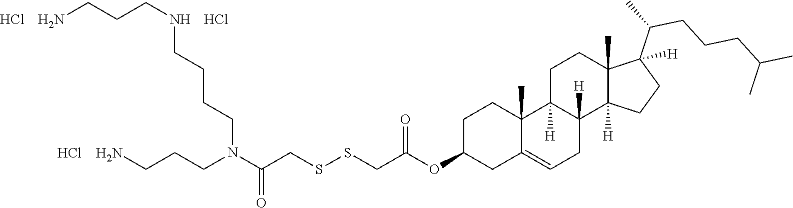

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 12-(tert-butoxycarbonyl)-7-(3-((tert-butoxycarbonyl)amino)propyl)-19,19-dimethyl-6,17-dioxo-18-oxa-3,4-dithia-7,12,16-triazaicosanoate (0.33 g, 0.31 mmol) in isopropanol (5 mL) set stirring under nitrogen was added hydrochloric acid (5N in isopropanol, 0.63 mL, 3.14 mmol) dropwise. The solution was heated to 40° C. and allowed to proceed overnight. The following morning, the solution was cooled to room temperature, and dry acetonitrile (10 mL) was added to the mixture, which was sonicated and allowed to stir for an additional hour. The white solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((3-aminopropyl)(4-((3-aminopropyl)amino)butyl)amino)-2-oxoethyl)disulfaneyl)acetate trihydrochloride as a white solid (0.21 g, 0.22 mmol, 70.0%). UPLC/ELSD: RT=2.00 min. MS (ES): m/z (MH⁺) 735.7 for C₄₁H₇₇Cl₃N₄O₃S₂. ¹H NMR (300 MHz, MeOD) δ: ppm 5.43 (m, 1H), 4.60 (br. m, 1H), 3.90 (m, 2H), 3.67 (s, 2H), 3.53 (m, 5H), 3.33 (s, 2H), 3.11 (m, 9H), 2.39 (m, 2H), 1.98 (br. m, 10H), 1.55 (br. m, 13H), 1.39 (m, 7H), 1.18 (br. m, 6H), 1.08 (s, 6H), 0.98 (d, 4H, J=6 Hz), 0.91 (d, 6H, J=6 Hz), 0.75 (s, 3H).

### BC. Compound SA110: N-(8-Aminooctyl)-N-(3-aminopropyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamide dihydrochloride

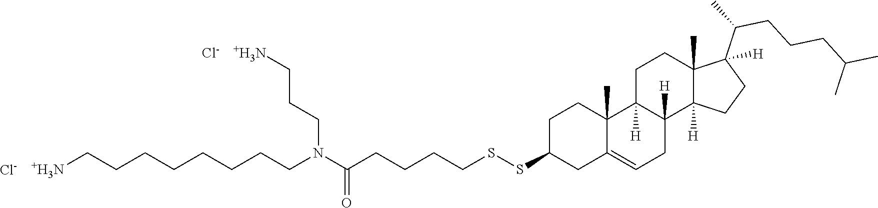

### Step 1: 5-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanoic acid

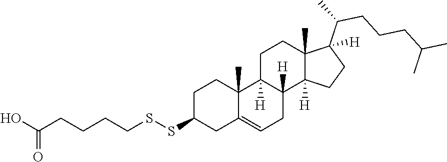

To a stirred mixture of 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (1.000 g, 1.479 mmol) in dimethylformamide (4.5 mL) was added 5-sulfanylpentanoic acid (0.208 g, 1.55 mmol) in dimethylformamide (2.0 mL). The reaction mixture stirred at rt and was monitored by LCMS. At 15 h additional 5-sulfanylpentanoic acid (60 mg) in dimethylformamide (0.5 mL) was added. At 40 h water (20 mL) was added, and the reaction mixture stirred at rt for 15 min and then was sonicated. Solids were collected by vacuum filtration and were rinsed with water. The solids were dissolved in dichloromethane, passed through a hydrophobic frit, dried over Na₂SO₄, and concentrated. Acetonitrile (25 mL) was added to the residue, and the suspension was sonicated. Solids were collected by vacuum filtration rinsing sparingly with cold acetonitrile to afford 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanoic acid (0.604 g, 1.13 mmol, 76.3%) as a white solid. UPLC/ELSD: RT=3.47 min; ¹H NMR (300 MHz, CDCl₃): δ 10.10 (br. s, 11H), 5.30-5.48 (m, 11H), 2.57-2.77 (m, 3H), 2.22-2.46 (m, 4H), 0.94-2.08 (br. m, 30H), 1.01 (s, 3H), 0.92 (d, 3H, J=6.5 Hz), 0.87 (d, 3H, J=6.6 Hz), 0.86 (d, 3H, J=6.6 Hz), 0.68 (s, 3H).

### Step 2: tert-Butyl (8-(N-(3-((tert-butoxycarbonyl)amino)propyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-JH-cyclopenta[a]phenanthren-3-yl)disulfaneyl)pentanamido)octyl)carbamate

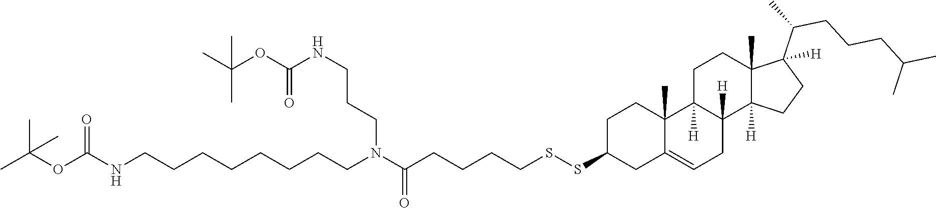

To a mixture of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanoic acid (0.250 g, 0.467 mmol), tert-butyl N-[3-({8-[(tert-butoxycarbonyl)amino]octyl}amino)propyl]carbamate (0.263 g, 0.654 mmol), and N-hydroxysuccinimide (0.081 g, 0.70 mmol) in dichloromethane (7.5 mL) was added dicyclohexylcarbodiimide (0.164 g, 0.795 mmol). The reaction mixture stirred at rt and was monitored by LCMS. At 50 h N-hydroxysuccinimide (34 mg) and dicyclohexylcarbodiimide (72 mg) were added. At 92 h hexanes (38 mL) was added, and then the reaction mixture was filtered through a pad of Celite rinsing with 5:1 hexanes/dichloromethane. The filtrate was concentrated and then purified via silica gel chromatography (10-50% ethyl acetate in hexanes) to afford tert-butyl (8-(N-(3-((tert-butoxycarbonyl)amino)propyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamido)octyl)carbamate (0.210 g, 0.229 mmol, 48.9%) as a clear oil. UPLC/ELSD: RT=3.53 min. MS (ES): m/z=919.93 [M+H]⁺ for C₅₃H₉₅N₃O₅S₂; ¹H NMR (300 MHz, CDCl₃): δ 5.23-5.48 (m, 2H), 4.38-4.67 (m, 1H), 2.99-3.45 (br. m, 8H), 2.56-2.76 (m, 3H), 2.22-2.41 (m, 4H), 0.93-2.08 (br. m, 44H), 1.44 (s, 9H), 1.43 (s, 9H), 1.00 (s, 3H), 0.91 (d, 3H, J=6.5 Hz), 0.87 (d, 3H, J=6.5 Hz), 0.86 (d, 3H, J=6.6 Hz), 0.68 (s, 3H).

### Step 3: N-(8-Aminooctyl)-N-(3-aminopropyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamide dihydrochloride

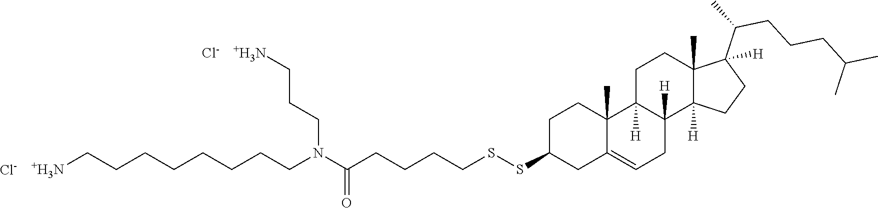

To a mixture of tert-butyl (8-(N-(3-((tert-butoxycarbonyl)amino)propyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamido)octyl)carbamate (0.208 g, 0.226 mmol) in isopropanol (3.0 mL) was added 5-6 N HCl in isopropanol (0.32 mL). The reaction mixture stirred at 40° C. and was monitored by LCMS. At 24 h the reaction mixture was diluted with methanol (3 mL) and filtered rinsing with methanol. The filtrate was concentrated, and then the residue was triturated with 19:1 acetonitrile/ethanol (2×3 mL). The residue was dissolved in methanol and then concentrated to afford N-(8-aminooctyl)-N-(3-aminopropyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamide dihydrochloride as a white foam (0.111 g, 0.140 mmol, 62.0%). UPLC/ELSD: RT=2.28 min. MS (ES): m/z=359.81 [M+2H]²⁺ for C₄₃H₇₉N₃OS₂.

¹H NMR (300 MHz, DMSO, reported as seen in spectrum): δ 7.67-8.29 (m, 8.78H), 5.25-5.43 (m, 1H), 3.14-3.43 (m, 7.91H), 2.58-2.86 (m, 10.97H), 2.17-2.39 (m, 5.35H), 0.79-2.07 (br m, 84.51H), 0.61-0.70 (m, 3.34H).

### BD. Compound SA111 (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride

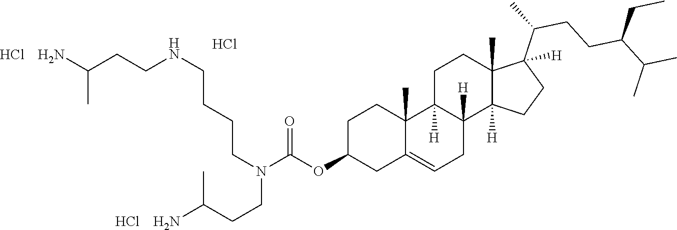

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-JH-cyclopenta[a]phenanthren-3-yl (3-((tert-butoxycarbonyl)amino)butyl)(4-((3-((tert-butoxycarbonyl)amino)butyl)amino)butyl)carbamate

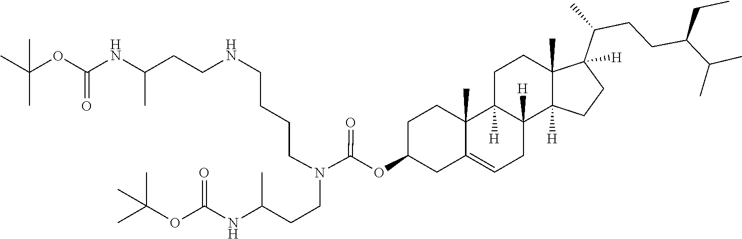

To a solution of di-tert-butyl ((butane-1,4-diylbis(azanediyl))bis(butane-4,2-diyl))dicarbamate (0.33 g, 0.77 mmol) in dry toluene (10 mL) set stirring under nitrogen was added triethylamine (0.32 mL, 2.30 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.44 g, 0.77 mmol) was added and the solution was heated to 90° C. and allowed to proceed overnight. The following morning, the reaction mixture was allowed to cool to room temperature, diluted with toluene, and washed with water (3×20 mL), dried over sodium sulfate, filtered, and concentrated to an oil. The oil was taken up in DCM and purified via silica gel chromatography in DCM with a 0-70% (70:25:5 DCM/MeOH/concentrated aqueous ammonium hydroxide) gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate as a light yellow oil (0.41 g, 0.48 mmol, 62.0%). UPLC/ELSD: RT=2.76 min. MS (ES): m/z (MH⁺) 872.3 for C₅₂H₉₄N₄O₆. ¹H NMR (300 MHz, CDCl₃) δ: ppm 5.08 (m, 1H), 4.67 (br. m, 1H), 4.19 (br. m, 2H), 3.42 (m, 2H), 3.13 (s, 3H), 2.90 (br. m, 4H), 2.29 (m, 4H), 2.05 (m, 4H), 1.69 (m, 6H), 1.35 (br. m, 14H), 1.14 (br. s, 17H), 0.99 (br. m, 6H), 0.86 (d, 9H, J=6 Hz), 0.73 (s, 5H), 0.64 (d, 5H, J=6 Hz), 0.55 (q, 8H, J=6 Hz), 0.38 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride

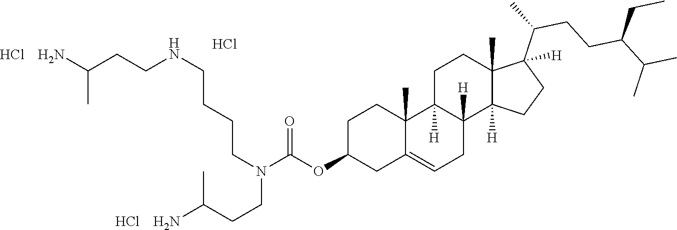

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate (0.41 g, 0.48 mmol) in isopropanol (7 mL) set stirring under nitrogen was added hydrochloric acid (5N in isopropanol, 0.95 mL, 4.75 mmol) dropwise. The solution was heated to 40° C. and allowed to proceed overnight. The following morning, dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride as a white solid (0.30 g, 0.36 mmol, 74.8%). UPLC/ELSD: RT=1.50 min. MS (ES): m/z (MH⁺) 672.3 for C₄₂H₈₁Cl₃N₄O₂. ¹H NMR (300 MHz, MeOD) δ: ppm 5.41 (m, 1H), 4.48 (br. m, 1H), 3.48 (br. m, 2H), 3.33 (s, 7H), 3.17 (m, 3H), 2.39 (d, 2H, J=3 Hz), 1.92 (br. m, 8H), 1.73 (br. m, 10H), 1.37 (br. m, 9H), 1.17 (d, 4H, J=6 Hz), 1.07 (s, 5H), 0.98 (d, 5H, J=6 Hz), 0.86 (q, 8H, J=6 Hz), 0.74 (s, 3H).

### BE. Compound SA113: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride

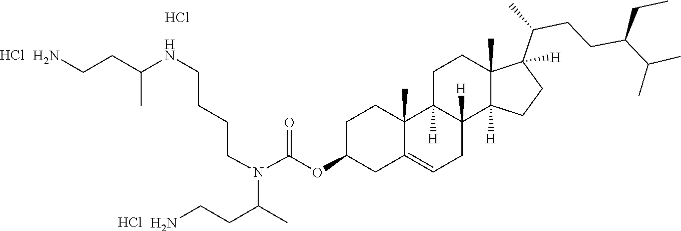

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate

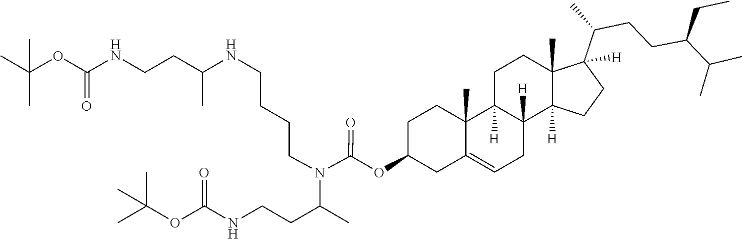

To a solution of di-tert-butyl ((butane-1,4-diylbis(azanediyl))bis(butane-3,1-diyl))dicarbamate (0.19 g, 0.43 mmol) in dry toluene (10 mL) set stirring under nitrogen was added triethylamine (0.18 mL, 1.31 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.25 g, 0.44 mmol) was added. The solution was heated to 90° C. and allowed to proceed for 48 h. Then, the reaction mixture was allowed to cool to room temperature, diluted with toluene, washed with water (3×20 mL), dried over sodium sulfate, filtered, and concentrated to give an oil. The oil was taken up in DCM and purified via silica gel chromatography in DCM with a 0-80% (70:25:5 DCM/MeOH/concentrated aqueous ammonium hydroxide) gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate as a light yellow oil (0.16 g, 0.18 mmol, 41.0%). UPLC/ELSD: RT=2.50 min. MS (ES): m/z (MH⁺) 872.3 for C₅₂H₉₄N₄O₆. ¹H NMR (300 MHz, CDCl₃) δ: ppm 5.34 (br. m, 2H), 4.48 (br. m, 1H), 4.17 (br. m, 1H), 3.15 (br. m, 6H), 2.48 (br. m, 7H), 1.95 (br. m, 7H), 1.51 (br. m, 15H), 1.39 (s, 25H), 1.11 (br. m, 15H), 1.04 (d, 5H, J=6 Hz), 0.98 (s, 6H), 0.89 (d, 6H, J=6 Hz), 0.78 (q, 10H, J=6 Hz), 0.64 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride

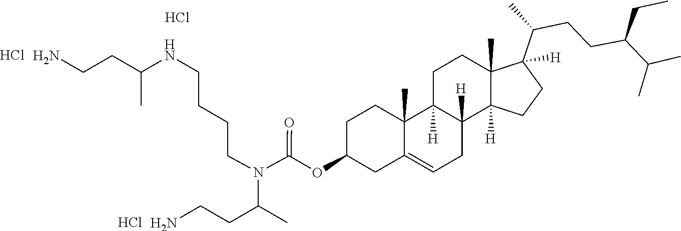

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate (0.16 g, 0.18 mmol) in isopropanol (5 mL) set stirring under nitrogen was added hydrochloric acid (5N in isopropanol, 0.36 mL, 1.80 mmol) dropwise. The solution was heated to 40° C. and allowed to proceed overnight. The following morning, dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride as a white solid (0.11 g, 0.13 mmol, 72.0%). UPLC/ELSD: RT=1.69 min. MS (ES): m/z (MH⁺) 673.2 for C₄₂H₈₁Cl₃N₄O₂. ¹H NMR (300 MHz, MeOD) δ: ppm 5.43 (m, 1H), 4.47 (br. m, 1H), 4.12 (m, 1H), 3.43 (br. m, 1H), 3.33 (s, 4H), 3.24 (br. m, 2H), 3.13 (m, 5H), 2.91 (br. m, 2H), 2.41 (d, 2H, J=3 Hz), 1.99 (br. m, 10H), 1.76 (br. m, 12H), 1.43 (d, 6H, J=6 Hz), 1.31 (d, 6H, J=6 Hz), 1.18 (br. m, 6H), 1.08 (s, 6H), 0.99 (d, 5H, J=6 Hz), 0.89 (q, 9H, J=6 Hz), 0.75 (s, 3H).

### BF. Compound SA114 (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride

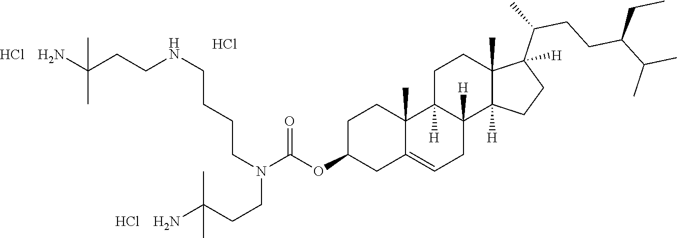

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate

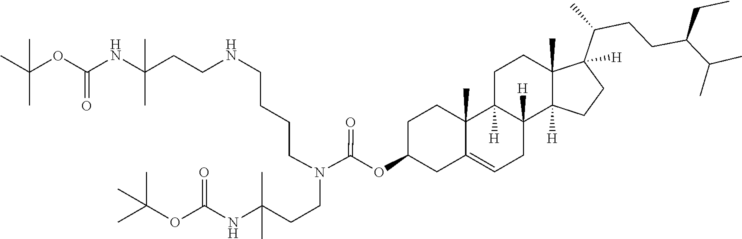

To a solution of di-tert-butyl ((butane-1,4-diylbis(azanediyl))bis(2-methylbutane-4,2-diyl))dicarbamate (0.30 g, 0.65 mmol) in dry toluene (10 mL) set stirring under nitrogen was added triethylamine (0.32 mL, 2.30 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.38 g, 0.65 mmol) was added and the solution was heated to 90° C. and allowed to proceed overnight. The following morning, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in DCM with a 0-70% (70:25:5 DCM/MeOH/concentrated aqueous ammonium hydroxide) gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate as a light yellow oil (0.36 g, 0.40 mmol, 60.5%). UPLC/ELSD: RT=2.86 min. MS (ES): m/z (MH⁺) 900.3 for C₅₄H₉₈N₄O₆. ¹H NMR (300 MHz, CDCl₃) S: ppm 5.63 (m, 1H), 5.09 (m, 1H), 5.01 (s, 1H), 4.22 (br. m, 2H), 2.93 (br. m, 4H), 2.40 (t, 2H), 2.32 (t, 2H), 2.05 (br. m, 2H), 1.59 (br. m, 7H), 1.26 (br. m, 13H), 1.14 (s, 20H), 1.02 (d, 16H, J=9 Hz), 0.84 (br. m, 9H), 0.73 (s, 6H), 0.65 (d, 6H, J=6 Hz), 0.56 (q, 10H, J=6 Hz), 0.39 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride

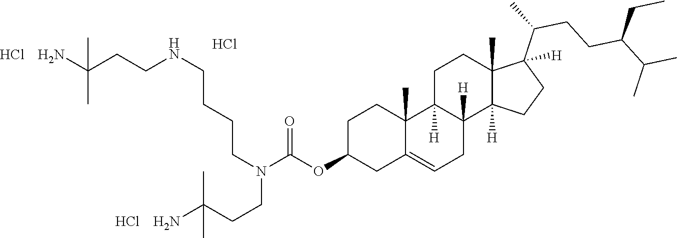

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate (0.36 g, 0.40 mmol) in isopropanol (7 mL) set stirring under nitrogen was added hydrochloric acid (5N in isopropanol, 0.79 mL, 3.96 mmol) dropwise. The solution was heated to 40° C. and allowed to proceed overnight. The following morning, dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride as a white solid (0.24 g, 0.27 mmol, 69.3%). UPLC/ELSD: RT=1.50 min. MS (ES): m/z (MH⁺) 700.3 for C₄₄H₈₅Cl₃N₄O₂. ¹H NMR (300 MHz, MeOD) δ: ppm 5.42 (m, 1H), 4.44 (br. m, 1H), 3.94 (m, 1H), 3.48 (br. m, 2H), 3.33 (br. m, 8H), 3.15 (m, 4H), 2.40 (d, 2H, J=3 Hz), 2.12 (br. m, 10H), 1.74 (br. m, 12H), 1.42 (d, 16H, J=6 Hz), 1.18 (d, 11H, J=6 Hz), 1.08 (s, 6H), 0.98 (d, 5H, J=6 Hz), 0.87 (q, 9H, J=6 Hz), 0.75 (s, 3H).

### BG. Compound SA116: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride

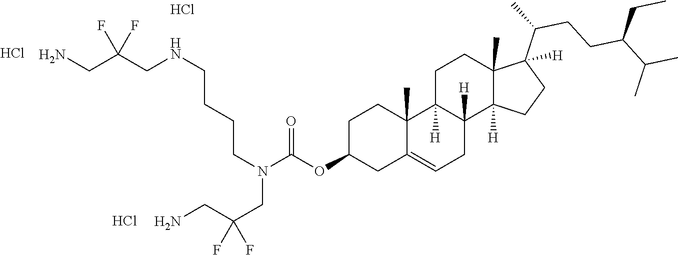

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate

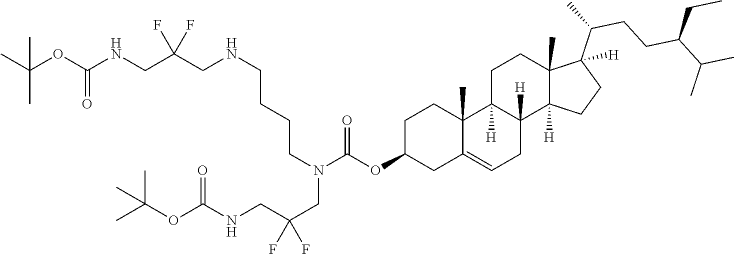

To a solution of di-tert-butyl ((butane-1,4-diylbis(azanediyl))bis(2,2-difluoropropane-3,1-diyl))dicarbamate (0.37 g, 0.78 mmol) in dry toluene (10 mL) set stirring under nitrogen was added triethylamine (0.33 mL, 2.33 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.45 g, 0.78 mmol) was added. The solution was heated to 90° C. and allowed to proceed for 48 h. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in DCM with a 0-80% (70:25:5 DCM/MeOH/concentrated aqueous ammonium hydroxide) gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate as a light yellow oil (0.05 g, 0.06 mmol, 7.3%). UPLC/ELSD: RT=2.77 min. MS (ES): m/z (MH⁺) 916.3 for C₅₀H₈₆F₄N₄O₆. ¹H NMR (300 MHz, CDCl₃) δ: ppm 5.61 (br. m, 1H), 5.41 (br. m, 1H), 5.00 (br. m, 1H), 4.55 (br. m, 1H), 3.65 (br. m, 4H), 3.33 (br. m, 2H), 2.97 (t, 2H), 2.69 (t, 1H), 2.36 (br. m, 2H), 1.87 (br. m, 4H), 1.59 (br. m, 7H), 1.46 (s, 17H), 1.14 (br. m, 14H), 1.04 (s, 5H), 0.95 (d, 4H, J=6 Hz), 0.86 (q, 8H, J=6 Hz), 0.70 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride

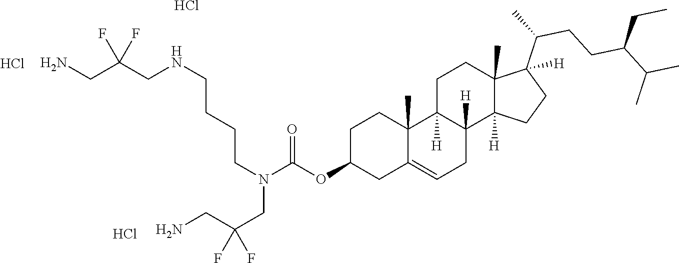

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate (0.05 g, 0.06 mmol) in isopropanol (5 mL) set stirring under nitrogen was added hydrochloric acid (5N in isopropanol, 0.11 mL, 0.57 mmol) dropwise. The solution was heated to 40° C. and allowed to proceed overnight. The following morning, dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride as a white solid (0.04 g, 0.04 mmol, 77.1%). UPLC/ELSD: RT=1.63 min. MS (ES): m/z (MH⁺) 716.1 for C₄₀H₇₃Cl₃F₄N₄O₂. ¹H NMR (300 MHz, MeOD) δ: ppm 5.32 (m, 1H), 4.38 (br. m, 1H), 3.81 (br. m, 6H), 3.33 (br. m, 4H), 3.22 (s, 5H), 3.08 (br. m, 2H), 2.28 (d, 2H, J=3 Hz), 1.93 (br. m, 5H), 1.54 (br. m, 9H), 1.26 (br. m, 6H), 1.06 (d, 6H, J=6 Hz), 0.97 (s, 5H), 0.87 (d, 4H, J=6 Hz), 0.77 (q, 7H, J=6 Hz), 0.63 (s, 3H).

### BH. Compound SA117: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride

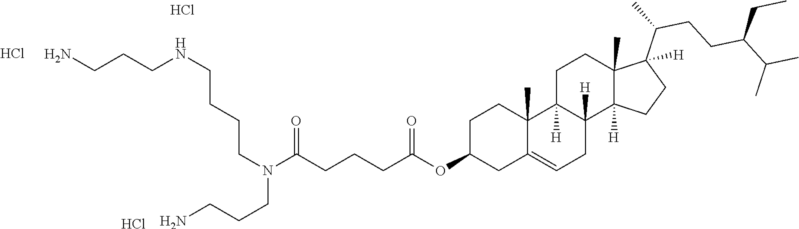

### Step 1: 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid

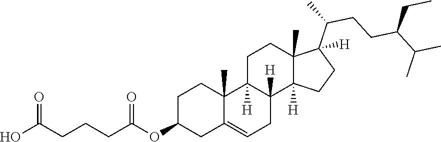

To a solution of sitosterol (2.50 g, 5.71 mmol) in acetone (30 mL) stirring under nitrogen was added glutaric anhydride (1.17 g, 10.28 mmol) and triethylamine (1.43 mL, 10.28 mmol). The reaction mixture was refluxed at 56° C., turning from a white slurry to a colorless clear solution, and allowed to proceed at reflux for 3 days. The solution was then cooled to room temperature, concentrated under vacuum, and taken up in 150 mL dichloromethane. This was then washed with 0.5 M HCl (1×100 mL), saturated aqueous ammonium chloride (1×100 mL), and water (1×100 mL), dried over sodium sulfate, filtered, and concentrated to a white solid. The solid was taken up in dichloromethane and purified on silica with a 0-80% ethyl acetate gradient in hexanes to give 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid as a white solid (2.33 g, 4.40 mmol, 77.1%). UPLC/ELSD: RT: 3.30 min. MS (ES): m/z (MH⁺) 529.8 for C₃₄H₅₆O₄. ¹H NMR (300 MHz, CDCl₃) δ: ppm 5.38 (m, 1H), 3.53 (m, 1H), 2.31 (br. m, 3H), 2.07 (br. m, 3H), 1.98 (br. m, 3H), 1.50 (br. m, 7H), 1.26 (br. m, 12H), 1.03 (s, 5H), 0.93 (d, 6H, J=6 Hz), 0.85 (q, 10H, J=6 Hz), 0.70 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate

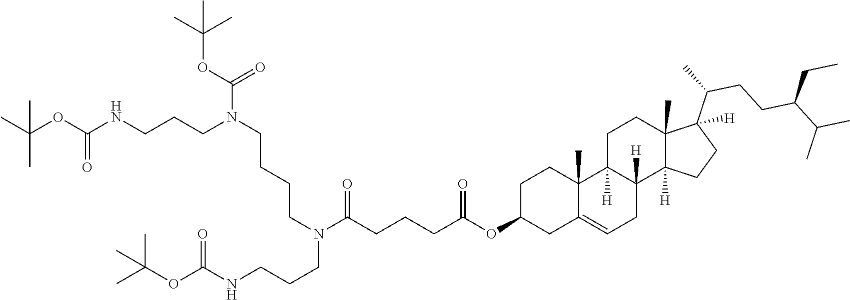

To a solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (1.50 g, 2.81 mmol) in dry DCM (25 mL) stirring under nitrogen was added tert-butyl (3-((tert-butoxycarbonyl)amino)propyl)(4-((3-((tert-butoxycarbonyl)amino)propyl)amino)butyl)carbamate (1.41 g, 2.81 mmol), dimethylaminopyridine (0.04 g, 0.28 mmol), and 1-ethyl-3-(3-dimethylaminopropyl)carbodiimide hydrochloride (1.09 g, 5.62 mmol). The resulting solution was cooled to 0° C. and diisopropylethylamine (1.49 mL, 8.42 mmol) was added dropwise. The mixture was allowed to gradually warm to room temperature and proceed for 48 h. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-80% (70:25:5 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate as a light yellow oil (0.41 g, 0.40 mmol, 14.3%). UPLC/ELSD: RT: 3.29 min. MS (ES): m/z (MH⁺) 1014.5 for C₄₉H₁₀₄N₄O₉. ¹H NMR (300 MHz, CDCl₃) δ: ppm 5.41 (m, 2H), 4.52 (br. m, 1H), 4.14 (m, 1H), 3.16 (br. m, 13H), 2.29 (br. m, 7H), 1.75 (br. m, 21H), 1.35 (d, 33H, J=6 Hz), 1.09 (br. m, 12H), 0.93 (s, 7H), 0.85 (d, 5H, J=6 Hz), 0.76 (q, 9H, J=6 Hz), 0.59 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride

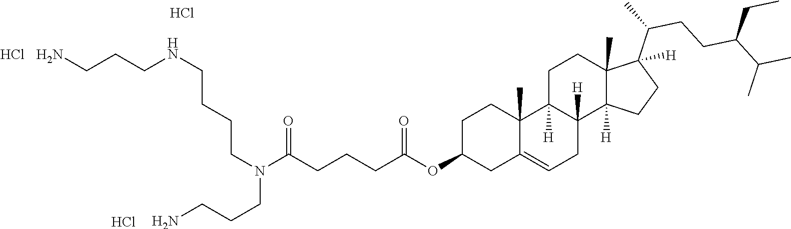

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate (0.41 g, 0.40 mmol) in isopropanol (5 mL) set stirring under nitrogen was added hydrochloric acid (5N in isopropanol, 0.80 mL, 4.01 mmol) dropwise. The solution was heated to 40° C. and allowed to proceed overnight. The following morning, dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride as a white solid (0.24 g, 0.27 mmol, 66.1%). UPLC/ELSD: RT=1.64 min. MS (ES): m/z (MH⁺) 714.3 for C₄₄H₈₃Cl₃N₄O₃. ¹H NMR (300 MHz, MeOD) δ: ppm 5.40 (m, 1H), 4.56 (br. m, 1H), 3.95 (m, 1H), 3.52 (br. m, 3H), 3.33 (s, 3H), 3.15 (br. m, 6H), 2.42 (br. m, 5H), 1.91 (br. m, 10H), 1.54 (br. m, 7H), 1.32 (br. m, 7H), 1.17 (d, 4H, J=6 Hz), 1.06 (s, 4H), 0.97 (d, 4H, J=6 Hz), 0.88 (q, 7H, J=6 Hz), 0.74 (s, 3H).

### BI. Compound SA119: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N₃-amino-aminopropyl)amino)butyl)glycinate tetrahydrochloride

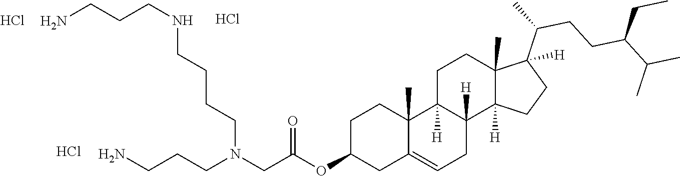

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloroacetate

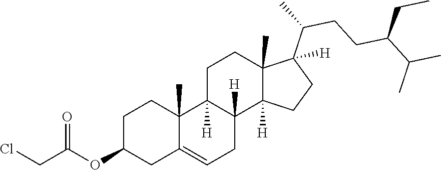

To a solution of sitosterol (2.00 g, 2.57 mmol) in dry dichloroethane (22.84 mL) was added DBU (2.05 mL, 13.70 mmol). The reaction was cooled to 0° C., and a solution of chloroacetyl chloride (0.73 mL, 9.13 mmol) in 5 mL dichloroethane was added to the reaction mixture dropwise, causing a change from a clear colorless solution to a cloudy dark brown mixture. The mixture was allowed to gradually warm to room temperature and stir overnight. The following morning, TLC suggested incomplete reaction progress, so the mixture was again cooled to 0° C. and an additional 0.50 mL DBU and 0.20 mL chloroacetyl chloride were added. The mixture warmed to room temperature, and the reaction was complete by TLC after 2 hours. The mixture was cooled to 0° C. again, and 30 mL water was added. Upon warming to room temperature, the aqueous layer was separated and washed with DCM (3×30 mL), and all organic layers were combined, dried over sodium sulfate, filtered, and concentrated to give a brown oil. The oil was taken up in DCM and purified on silica in hexanes with a 0-20% EtOAc gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloroacetate as a white solid (1.40 g, 2.84 mmol, 62.2%). UPLC/ELSD: RT: 3.49 min. MS (ES): m/z (MH⁺) 492.2 for C₃₁H₅₁ClO₂. ¹H NMR (300 MHz, CDCl₃) δ: ppm 5.41 (m, 1H), 4.70 (br. m, 1H), 4.06 (s, 2H), 2.40 (d, 2H, J=6 Hz), 1.93 (br. m, 5H), 1.52 (br. m, 7H), 1.20 (br. m, 11H), 1.05 (s, 6H), 0.96 (d, 5H, J=6 Hz), 0.85 (q, 9H, J=6 Hz), 0.70 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate

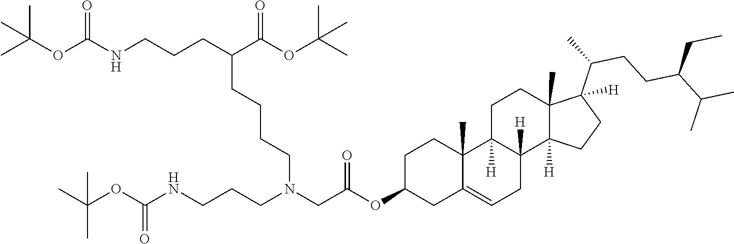

Both (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloroacetate (0.51 g, 1.04 mmol) and tert-butyl (3-((tert-butoxycarbonyl)amino)propyl)(4-((3-((tert-butoxycarbonyl)amino)propyl)amino)butyl)carbamate (0.71 g, 1.41 mmol) were combined in a vial and purged with three cycles of vacuum and nitrogen. Then, they were taken up in dry THF (10.42 mL), and triethylamine (0.29 mL, 2.09 mmol) was added. The mixture was set stirring under nitrogen, heated to 65° C., and allowed to stir for 48 h. Then, the mixture was cooled to room temperature and diluted with ethyl acetate (30 mL) and saturated aq. sodium bicarbonate (30 mL). The aqueous layer was separated and extracted with EtOAc (3×30 mL). All organic layers were combined, washed with brine (1×20 mL), dried over sodium sulfate, filtered, and concentrated to a yellow oil. The oil was taken up in DCM and purified on silica in hexanes with a 0-70% EtOAc gradient. Product-containing fractions were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate as a light yellow oil (0.47 g, 0.49 mmol, 46.8%). UPLC/ELSD: RT: 2.90 min. MS (ES): m/z (MH⁺) 958.4 for C₅₆H₁₀₀N₄O₈. ¹H NMR (300 MHz, CDCl₃) δ: ppm 5.39 (m, 1H), 4.64 (br. m, 1H), 4.14 (m, 1H), 3.26 (br. m, 9H), 2.60 (m, 4H), 2.35 (d, 2H, J=6 Hz), 2.05 (br. m, 6H), 1.65 (br. m, 8H), 1.47 (br. s, 30H), 1.20 (br. m, 11H), 1.03 (s, 5H), 0.95 (d, 5H, J=6 Hz), 0.86 (q, 8H, J=6 Hz), 0.69 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride

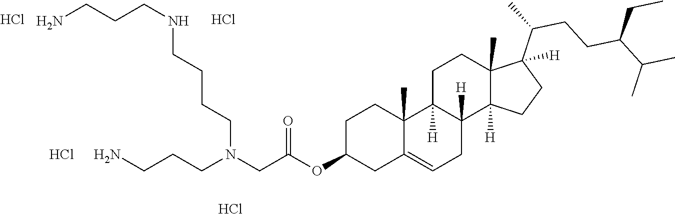

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate (0.47 g, 0.49 mmol) in isopropanol (7 mL) set stirring under nitrogen was added hydrochloric acid (5N in isopropanol, 1.17 mL, 5.85 mmol) dropwise. The solution was heated to 40° C. and allowed to proceed overnight. The following morning, the solution was cooled to room temperature and dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride as a white solid (0.36 g, 0.41 mmol, 83.5%). UPLC/ELSD: RT=1.71 min. MS (ES): m/z (MH⁺) 658.1 for C₄₁H₈₀Cl₄N₄O₂. ¹H NMR (300 MHz, MeOD) δ: ppm 5.42 (m, 1H), 4.73 (br. m, 2H), 4.30 (br. m, 2H), 3.93 (m, 1H), 3.32 (br. m, 6H), 3.10 (br. m, 8H), 2.43 (br. s, 2H), 2.17 (br. m, 4H), 2.03 (br. m, 10H), 1.53 (br. m, 8H), 1.31 (br. m, 9H), 1.15 (d, 6H, J=6 Hz), 1.05 (s, 6H), 0.95 (d, 5H, J=6 Hz), 0.86 (q, 9H, J=6 Hz), 0.72 (s, 3H).

### BJ. Compound SA120: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutyl)(3-aminopropyl)carbamate dihydrochloride

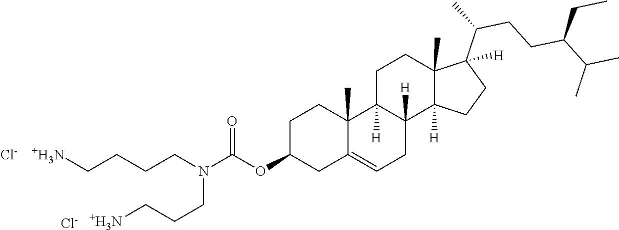

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butyl)(3-((tert-butoxycarbonyl)amino)propyl)carbamate

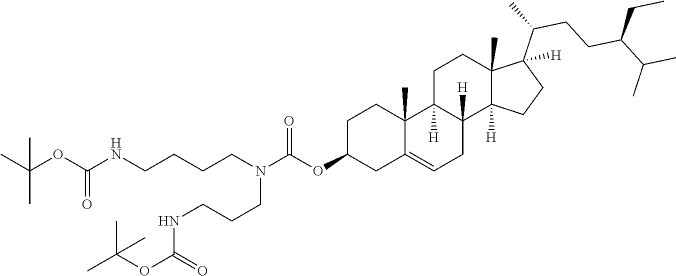

β-Sitosterol 4-nitrophenyl carbonate (0.200 g, 0.345 mmol), tert-butyl N-[3-({4-[(tert-butoxycarbonyl)amino]butyl}amino)propyl]carbamate (0.155 g, 0.448 mmol), and triethylamine (0.15 mL, 1.1 mmol) were combined in toluene (3.5 mL). The reaction mixture stirred at 90° C. and was monitored by LCMS. At 21 h the reaction mixture was cooled to rt, diluted with dichloromethane (20 mL), and washed with 5% aq. NaHCO₃ solution (3×10 mL). The organics were passed through a hydrophobic frit, dried over Na₂SO₄, and concentrated. The crude material was purified via silica gel chromatography (10-50% ethyl acetate in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butyl)(3-((tert-butoxycarbonyl)amino)propyl)carbamate (0.210 g, 0.267 mmol, 77.4%) as a white foam. UPLC/ELSD: RT=3.37 min. MS (ES): m/z=787.67 [M+H]⁴ for C₄₇H₈₃N₃O₆; ¹H NMR (300 MHz, CDCl₃): δ 5.10-5.44 (m, 2H), 4.42-4.89 (m, 2H), 3.01-3.40 (br. m, 8H), 2.22-2.42 (m, 2H), 1.76-2.09 (m, 5H), 0.88-1.75 (br. m, 28H), 1.44 (s, 18H), 1.02 (s, 3H), 0.92 (d, 3H, J=6.4 Hz), 0.78-0.88 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-JH-cyclopenta[a]phenanthren-3-yl (4-aminobutyl)(3-aminopropyl)carbamate dihydrochloride

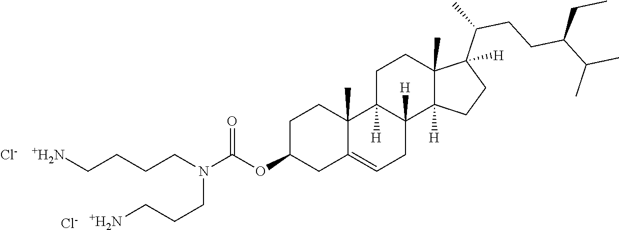

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butyl)(3-((tert-butoxycarbonyl)amino)propyl)carbamate (0.203 g, 0.258 mmol) in isopropanol (3.0 mL) was added 5-6 N HCl in isopropanol (0.37 mL). The reaction mixture stirred at 40° C. and was monitored by LCMS. At 24 h acetonitrile (9 mL) was added, and the reaction mixture stirred for 5 min. After this time solids were collected by vacuum filtration rinsing with 3:1 acetonitrile/isopropanol to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutyl)(3-aminopropyl)carbamate dihydrochloride (0.151 g, 0.218 mmol, 84.4%) as a white solid. UPLC/ELSD: RT=1.93 min. MS (ES): m/z=586.67 [M+H]⁺ for C₃₇H₆₇N₃O₂; ¹H NMR (300 MHz, CD₃OD): δ 5.38-5.47 (m, 1H), 4.39-4.55 (m, 1H), 3.28-3.46 (m, 4H), 2.91-3.06 (m, 4H), 2.31-2.47 (m, 2H), 1.83-2.14 (m, 7H), 0.93-1.79 (br. m, 26H), 1.08 (s, 3H), 0.98 (d, 3H, J=6.4 Hz), 0.82-0.93 (m, 9H), 0.75 (s, 3H).

### BK. Compound SA118: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate tetrahydrochloride

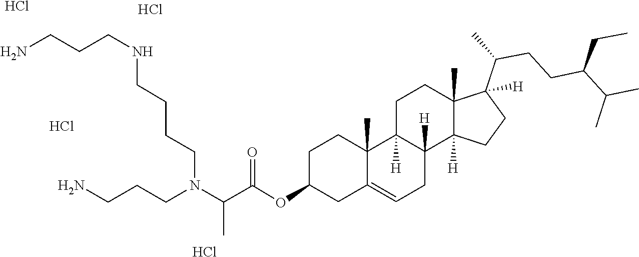

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloropropanoate

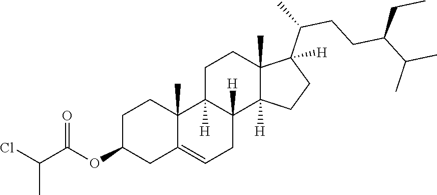

To a solution of β-sitosterol (1.25 g, 2.85 mmol) in dry DCM (15 mL) stirring under nitrogen was added 1,8-diazabicyclo[5.4.0]undec-7-ene (1.28 mL, 8.56 mmol). The reaction was cooled to 0° C., and a solution of 2-chloropropanoyl chloride (0.55 mL, 5.71 mmol) in 5 mL dry DCM was added dropwise over 20 minutes causing the solution to turn from a clear colorless mixture to a cloudy dark brown mixture. The reaction mixture was allowed to gradually warm to room temperature and proceed overnight. The following morning the reaction appeared to be incomplete via thin layer chromatography (7:3 hexanes/ethyl acetate, PMA stain), so the reaction mixture was again cooled to 0° C., and an additional 0.50 mL of 1,8-diazabicyclo[5.4.0]undec-7-ene was added. The reaction was allowed to warm to room temperature. After an hour, the reaction appeared complete by TLC. The reaction mixture was cooled to 0° C. and quenched with 20 mL of water. After the mixture warmed to room temperature, the layers were separated, and the aqueous layer was extracted with DCM (3×30 mL). All organic layers were combined, dried over sodium sulfate, filtered, and concentrated to a dark brown oil. The oil was taken up and purified on silica in hexanes with a gradient of 0-20% ethyl acetate. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloropropanoate as a white solid (1.12 g, 2.22 mmol, 77.7%). UPLC/ELSD: RT=3.39 min. MS (ES): m/z (MH⁺) 506.2 for C₃₂H₅₃C₁O₂. ¹H NMR (300 MHz, CDCl₃) δ: ppm 5.44 (m, 1H), 4.48 (br. m, 1H), 3.95 (m, 1H), 3.33 (br. m, 5H), 3.11 (br. m, 8H), 2.45 (br. m, 7H), 1.99 (br. m, 7H), 1.68 (br. m, 11H), 1.37 (br. m, 9H), 1.15 (d, 8H, J=6 Hz), 1.08 (s, 6H), 0.95 (d, 5H, J=6 Hz), 0.86 (q, 9H), 0.74 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2, 2,15-trimethyl-4-oxo-3-oxa-5,9,14-triazahexadecan-16-oate

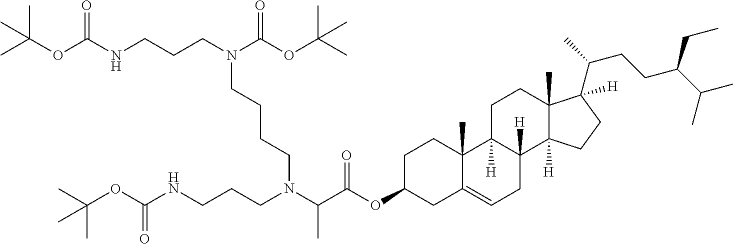

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloropropanoate (1.12 g, 2.22 mmol) and tert-butyl N-{3-[(tert-butoxycarbonyl)amino]propyl}-N-[4-({3-[(tert-butoxycarbonyl)amino]propyl}amino)butyl]carbamate (1.34 g, 2.66 mmol) in dry THF (22 ml) stirring under nitrogen was added triethylamine (0.62 mL, 4.43 mmol). The mixture was heated to 65° C. and allowed to proceed for a week, during which very little product was formed as monitored by LCMS. After a week, the mixture was cooled to room temperature and concentrated to an oil in vacuo. The oil was taken up in DCM and purified on silica in hexanes with a 0-50% ethyl acetate gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate as a light yellow oil (0.09 g, 0.10 mmol, 4.4%). UPLC/ELSD: RT=2.79 min. MS (ES): m/z (MH⁺) 972.5 for C₅₇H₁₀₂N₄O₈. ¹H NMR (300 MHz, CDCl₃) δ: ppm 5.31 (m, 1H), 4.54 (br. m, 1H), 3.39 (m, 1H), 3.07 (br. m, 7H), 2.52 (br. m, 4H), 2.23 (d, 2H, J=6 Hz), 1.77 (br. m, 6H), 1.54 (br. m, 9H), 1.36 (d, 30H, J=9 Hz), 1.17 (br. m, 14H), 0.95 (s, 5H), 0.86 (d, 5H, J=6 Hz), 0.75 (q, 8H), 0.61 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate tetrahydrochloride

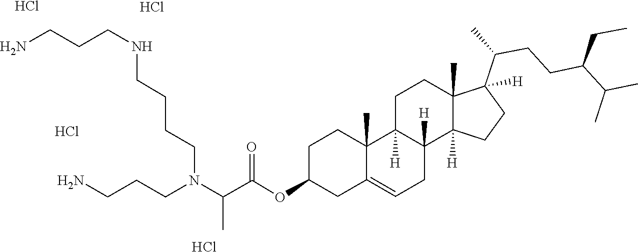

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate (0.09 g, 0.10 mmol) in isopropanol (5 mL) set stirring under nitrogen was added hydrochloric acid (5 N in isopropanol, 0.19 mL, 0.97 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate tetrahydrochloride as a white solid (0.07 g, 0.07 mmol, 76.9%). UPLC/ELSD: RT=1.61 min. MS (ES): m/z (MH⁺) 672.2 for C₄₂H₈₂Cl₄N₄O₂. ¹H NMR (300 MHz, MeOD) δ: ppm 5.44 (m, 1H), 4.48 (br. m, 1H), 3.95 (m, 1H), 3.32 (s, 5H), 3.11 (br. m, 8H), 2.27 (br. m, 7H), 1.99 (br. m, 7H), 1.68 (br. m, 11H), 1.37 (br. m, 9H), 1.15 (d, 8H, J=6 Hz), 1.08 (s, 6H), 0.95 (d, 5H, J=6 Hz), 0.86 (q, 9H), 0.74 (s, 3H).

### BL. Compound SA121: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride

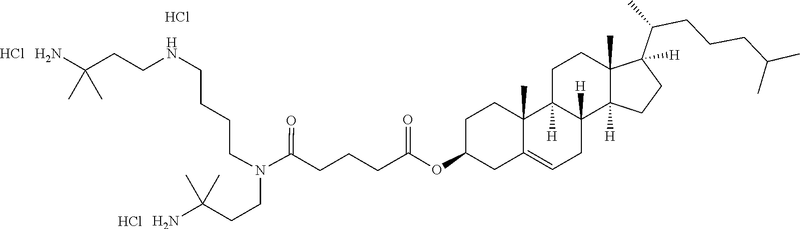

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate

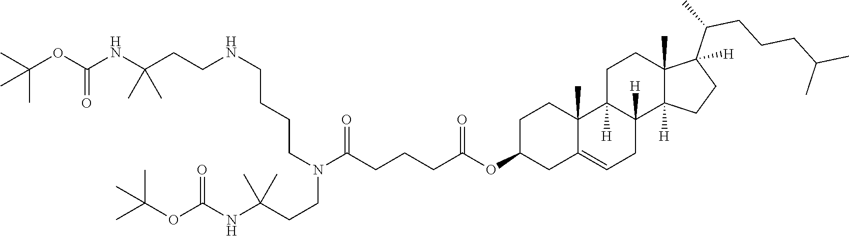

To a solution of 5-{[(1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl]oxy}-5-oxopentanoic acid (0.23 g, 0.45 mmol) in dry DCM (10 mL) stirring under nitrogen was added tert-butyl N-(4-{[4-({3-[(tert-butoxycarbonyl)amino]-3-methylbutyl}amino)butyl]amino}-2-methylbutan-2-yl)carbamate (0.23 g, 0.49 mmol), dimethylaminopyridine (0.01 g, 0.09 mmol), and 1-ethyl-3-(3-dimethylaminopropyl)carbodiimide hydrochloride (0.17 g, 0.89 mmol). The resulting solution was cooled to 0° C. and diisopropylethylamine (0.24 mL, 1.34 mmol) was added dropwise. The mixture was allowed to gradually warm to room temperature and proceed overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-80% (70:25:5 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate as a light yellow oil (0.09 g, 0.09 mmol, 20.2%). UPLC/ELSD: RT: 2.74 min. MS (ES): m/z (MH⁺) 942.4 for C₅₆H₁₀₀N₄O₇. ¹H NMR (300 MHz, CDCl₃) δ: ppm 5.30 (m, 1H), 4.52 (br. m, 2H), 3.23 (m, 4H), 2.93 (s, 1H), 2.61 (t, 2H) 2.54 (t, 2H), 2.29 (br. m, 6H), 1.87 (br. m, 10H), 1.62 (m, 3H), 1.49 (m, 6H), 1.36 (d, 24H, J=3 Hz), 1.23 (br. m, 17H), 1.06 (br. m, 7H), 0.94 (s, 7H), 0.85 (d, 4H, J=9 Hz), 0.81 (d, 7H, J=9 Hz), 0.61 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride

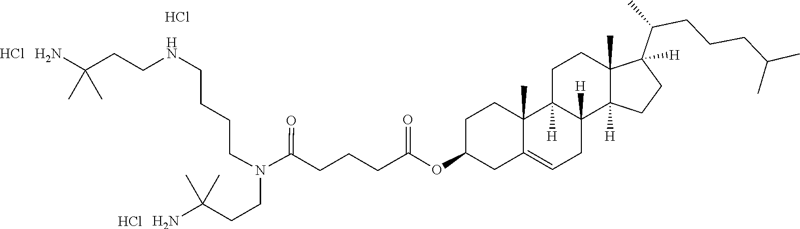

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate (0.06 g, 0.06 mmol) in isopropanol (5 mL) set stirring under nitrogen was added hydrochloric acid (5 N in isopropanol, 0.12 mL, 0.61 mmol) dropwise. The solution was heated to 42° C. and allowed to proceed overnight. The following morning, dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride as a white solid (0.03 g, 0.04 mmol, 58.0%). UPLC/ELSD: RT=1.62 min. MS (ES): m/z (MH⁺) 742.3 for C₄₆H₈₇Cl₃N₄O₃. ¹H NMR (300 MHz, MeOD) δ: ppm 5.42 (m, 1H), 4.57 (br. m, 1H), 3.48 (m, 4H), 3.33 (br. m, 3H), 3.16 (br. m, 4H), 2.48 (br. m, 5H), 2.14 (m, 2H), 1.91 (br. m, 10H), 1.54 (br. m, 6H), 1.42 (br. m, 14H), 1.16 (m, 6H), 1.06 (s, 5H), 0.97 (d, 3H, J=6 Hz), 0.91 (q, 5H, J=6 Hz), 0.74 (s, 3H).

### BM. Compound SA122: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(5-aminopentanoyl)glycinate dihydrochloride

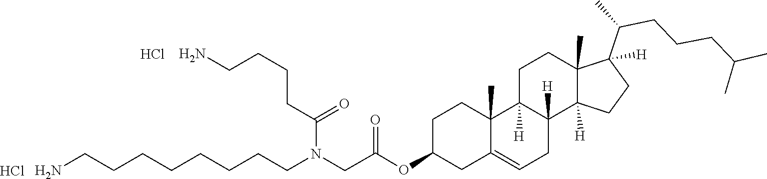

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate

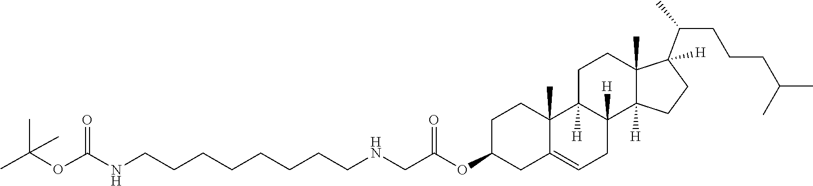

Tert-butyl N-(8-aminooctyl)carbamate (0.994 g, 4.07 mmol), cholesteryl chloroacetate (1.000 g, 2.159 mmol), potassium iodide (0.072 g, 0.43 mmol) and potassium carbonate (0.597 g, 4.32 mmol) were combined in dioxane (15 mL) in a sealed tube. The reaction mixture was monitored by LCMS. The reaction mixture was irradiated with microwaves at 140° C. for 3 h while stirring. The reaction mixture was irradiated with microwaves at 150° C. for 3 h while stirring, cooled to rt, and filtered through a pad of Celite rinsing with EtOAc. The filtrate was concentrated and then taken up in DCM (100 mL). The organics were washed with water, passed through a hydrophobic frit, dried over Na₂SO₄, and concentrated. The crude material was purified via silica gel chromatography (30-70% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.993 g, 1.48 mmol, 68.5%) as a viscous, amber oil. UPLC/ELSD: RT=2.56 min. MS (ES): m/z=672.06 [M+H]⁺ for C₄₂H₇₄N₂O₄. ¹H NMR (300 MHz, CDCl₃): δ 5.35-5.43 (m, 1H), 4.60-4.76 (m, 1H), 4.50 (br. s, 1H), 3.46 (s, 2H), 2.99-3.17 (m, 2H), 2.69 (t, 2H, J=7.3 Hz), 2.28-2.40 (m, 2H), 1.72-2.08 (m, 5H), 0.93-1.71 (br. m, 33H), 1.44 (s, 9H), 1.02 (s, 3H), 0.91 (d, 3H, J=6.5 Hz), 0.87 (d, 3H, J=6.5 Hz), 0.86 (d, 3H, J=6.6 Hz), 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(5-((tert-butoxycarbonyl)amino)pentanoyl)glycinate

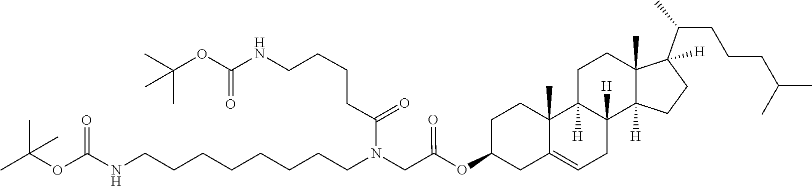

(3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.150 g, 0.224 mmol), 5-[(tert-butoxycarbonyl)amino]pentanoic acid (0.058 g, 0.268 mmol), and DMAP (cat.) were combined in DCM (3.0 mL). The reaction mixture was cooled to 0° C. in an ice bath and then 1-ethyl-3-(3-dimethylaminopropyl)carbodiimide hydrochloride (0.064 g, 0.34 mmol) was added. The reaction mixture stirred at rt and was monitored by LCMS. At 20 h, the reaction mixture was cooled to 0° C. in an ice bath, and then water (3 mL) was added. The biphasic mixture was diluted with DCM (5 mL). The layers were separated, and the aqueous was extracted with DCM (5 mL). The combined organics were passed through a hydrophobic frit, dried over Na₂SO₄, and concentrated. The crude material was purified via silica gel chromatography (20-60% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(5-((tert-butoxycarbonyl)amino)pentanoyl)glycinate (0.185 g, 0.213 mmol, 95.1%) as a clear oil. UPLC/ELSD: RT=3.27 min. MS (ES): m/z=872.43 [M+H]⁺ for C₅₂H₉₁N₃O₇. ¹H NMR (300 MHz, CDCl₃): δ 5.32-5.47 (m, 1H), 4.29-4.78 (m, 3H), 3.92-4.06 (m, 2H), 3.23-3.42 (m, 2H), 3.01-3.21 (m, 4H), 2.18-2.45 (m, 4H), 1.05-2.10 (br. m, 60H), 1.01 (s, 3H), 0.91 (d, 3H, J=6.2 Hz), 0.87 (d, 6H, J=6.5 Hz), 0.68 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(5-aminopentanoyl)glycinate dihydrochloride

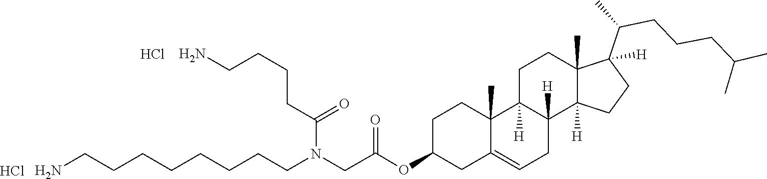

To a solution of (3S,8S,9S,1R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(5-((tert-butoxycarbonyl)amino)pentanoyl)glycinate (0.237 g, 0.272 mmol) in iPrOH (3.5 mL) was added 5-6 N HCl in iPrOH (0.39 mL). The reaction mixture was stirred at 40° C. and was monitored by LCMS. At 16 h, the reaction mixture was cooled to rt, and then ACN (10.5 mL) was added. Solids were collected by vacuum filtration rinsing with 3:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(5-aminopentanoyl)glycinate dihydrochloride (0.150 g, 0.185 mmol, 68.1%) as a white solid. UPLC/ELSD: RT=1.94 min. MS (ES): m/z=336.11 [M+2H]²⁺ for C₄₂H₇₅N₃O₃. ¹H NMR (300 MHz, DMSO): δ 7.90 (br. s, 6H), 5.31-5.40 (m, 11H), 4.39-4.61 (m, 1H), 3.89-4.24 (m, 2H), 3.18-3.39 (m, 2H), 2.65-2.88 (m, 4H), 2.13-2.41 (m, 4H), 1.70-2.05 (m, 5H), 0.91-1.67 (br. m, 37H), 0.98 (s, 3H), 0.89 (d, 3H, J=6.3 Hz), 0.84 (d, 6H, J=6.5 Hz), 0.65 (s, 3H).

### BN. Compound SA123: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-aminohexanoyl)-N-(8-aminooctyl)glycinate dihydrochloride

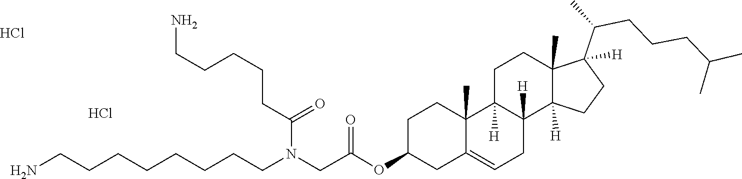

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-((tert-butoxycarbonyl)amino)hexanoyl)-N-(8-((tert-butoxycarbonyl)amino)octyl)glycinate

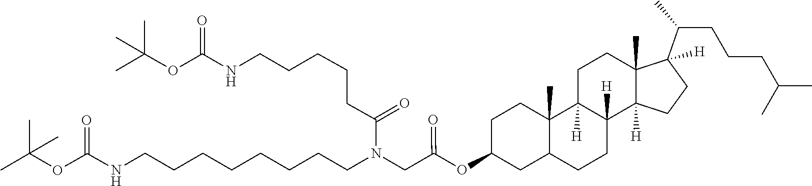

(3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.200 g, 0.298 mmol), 6-[(tert-butoxycarbonyl)amino]hexanoic acid (0.090 g, 0.39 mmol), and DMAP (cat.) were combined in DCM (4.0 mL). The reaction mixture was cooled to 0° C. in an ice bath, and 1-ethyl-3-(3-dimethylaminopropyl)carbodiimide hydrochloride (0.086 g, 0.45 mmol) was added. The reaction mixture was stirred at rt and was monitored by LCMS. At 20 h, the reaction mixture was cooled to 0° C. in an ice bath, and then water (4 mL) was added. The biphasic mixture was diluted with DCM (5 mL). The layers were separated, and the aqueous was extracted with DCM (5 mL). The combined organics were passed through a hydrophobic frit, dried over Na₂SO₄, and concentrated. The crude material was purified via silica gel chromatography (20-60% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-((tert-butoxycarbonyl)amino)hexanoyl)-N-(8-((tert-butoxycarbonyl)amino)octyl)glycinate (0.260 g, 0.294 mmol, 98.6%) as a clear oil. UPLC/ELSD: RT=3.28 min. MS (ES): m/z=885.39 [M+H]⁺ for C₅₃H₉₃N₃O₇. ¹H NMR (300 MHz, CDCl₃): δ 5.27-5.51 (m, 1H), 4.28-4.76 (m, 3H), 3.94-4.05 (m, 2H), 3.23-3.41 (m, 2H), 3.02-3.20 (m, 4H), 2.16-2.43 (m, 4H), 0.93-2.11 (br. m, 65H), 0.91 (d, 3H, J=6.5 Hz), 0.87 (d, 3H, J=6.6 Hz), 0.86 (d, 3H, J=6.6 Hz), 0.63-0.75 (m, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-aminohexanoyl)-N-(8-aminooctyl)glycinate dihydrochloride

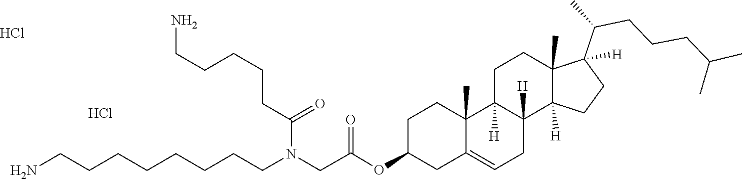

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-((tert-butoxycarbonyl)amino)hexanoyl)-N-(8-((tert-butoxycarbonyl)amino)octyl)glycinate (0.251 g, 0.284 mmol) in iPrOH (4.0 mL) was added 5-6 N HCl in iPrOH (0.40 mL). The reaction mixture stirred at 40° C. and was monitored by LCMS. At 16 h, the reaction mixture was cooled to rt, then ACN (12 mL) was added. Solids were collected by vacuum filtration rinsing with 3:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-aminohexanoyl)-N-(8-aminooctyl)glycinate dihydrochloride (0.163 g, 0.198 mmol, 69.8%) as a white solid. UPLC/ELSD: RT=1.97 min. MS (ES): m/z=343.02 [M+2H]²⁺ for C₄₃H₇₇N₃O₃. ¹H NMR (300 MHz, CD₃OD): δ 5.37-5.47 (m, 1H), 4.51-4.70 (m, 1H), 4.02-4.25 (m, 2H), 3.35-3.49 (m, 2H), 2.87-3.01 (m, 4H), 2.28-2.56 (m, 4H), 1.79-2.14 (m, 5H), 0.99-1.78 (br. m, 39H), 1.07 (s, 3H), 0.96 (d, 3H, J=6.4 Hz), 0.90 (d, 6H, J=6.6 Hz), 0.74 (s, 3H).

### BO. Compound SA124: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-aminocyclopropyl)ethyl)(4-((2-(1-aminocyclopropyl)ethyl)amino)butyl)carbamate trihydrochloride

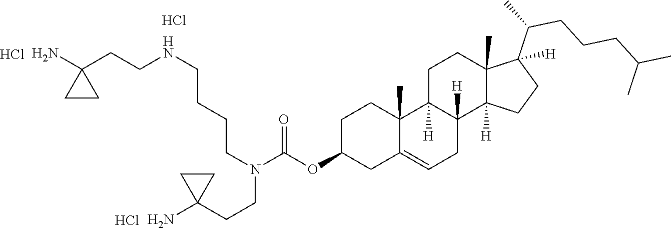

### Step 1: tert-butyl (1-(2-((2-nitrophenyl)sulfonamido)ethyl)cyclopropyl)carbamate

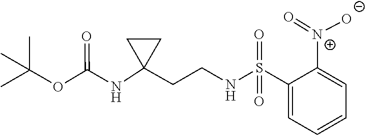 [SMILES: CC(C)(C)OC(=O)NC1(CCNS(=O)(=O)c2ccccc2[N+](=O)[O-])CC1]

To a solution of tert-butyl N-[1-(2-aminoethyl)cyclopropyl]carbamate (2.00 g, 9.49 mmol) in dry DCM (25 mL) set stirring under nitrogen was added triethylamine (2.64 mL, 18.98 mmol). The solution was cooled to 0° C., and then a solution of 2-nitrobenzenesulfonyl chloride (2.31 g, 10.44 mmol) in 25 mL dry DCM was added dropwise over 30 minutes. The reaction was allowed to proceed at 0° C. for an hour and then at room temperature for an additional three hours. Following, the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (1-(2-((2-nitrophenyl)sulfonamido)ethyl)cyclopropyl)carbamate as a white solid (3.73 g, 9.70 mmol, quantitative). UPLC/ELSD: RT=0.59 min. MS (ES): m/z (MH⁺) 386.4 for C₁₆H₂₃N₃O₆S. 1H NMR (300 MHz, CDCl₃) δ: ppm 8.13 (m, 1H), 7.84 (m, 1H), 7.73 (m, 2H), 6.45 (br. s, 1H), 4.89 (br. s, 1H), 3.27 (q, 2H), 1.73 (t, 2H), 1.38 (s, 9H), 0.82 (br. m, 2H), 0.69 (br. s, 2H).

### Step 2: di-tert-butyl (((butane-1,4-diylbis(azanediyl))bis(ethane-2,1-diyl))bis(cyclopropane-1,1-diyl))dicarbamate

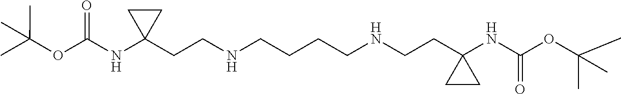 [SMILES: CC(C)(C)OC(=O)NC1(CCNCCCCNCCC2(NC(=O)OC(C)(C)C)CC2)CC1]

To a solution of tert-butyl (1-(2-((2-nitrophenyl)sulfonamido)ethyl)cyclopropyl)carbamate (3.74 g, 9.70 mmol) in dry DMF (50 mL) set stirring under nitrogen was added potassium carbonate (3.89 g, 28.16 mmol) and 1,4-diiodobutane (0.61 mL, 4.62 mmol). The solution was heated to 40° C. and allowed to proceed overnight. The following morning, benzyl bromide (0.46 mL, 3.83 mmol) was added, and the reaction was allowed to proceed at room temperature for 8 h. Then thiophenol (1.82 mL, 17.78 mmol), potassium carbonate (1.91 g, 13.85 mmol), and an additional 10 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl (((butane-1,4-diylbis(azanediyl))bis(ethane-2,1-diyl))bis(cyclopropane-1,1-diyl))dicarbamate as a colorless oil (0.47 g, 1.04 mmol, 22.5%). UPLC/ELSD: RT=0.35 min. MS (ES): m/z (MH⁺) 455.6 for C₂₄H₄₆N₄O₄. ¹H NMR (300 MHz, CDCl₃) δ: ppm 5.03 (m, 2H), 2.76 (t, 4H), 2.64 (m, 4H), 1.72 (m, 4H), 1.59 (m, 4H), 1.44 (s, 17H), 0.80 (m, 4H), 0.66 (m, 4H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-((tert-butoxycarbonyl)amino)cyclopropyl)ethyl)(4-((2-(1-((tert-butoxycarbonyl)amino)cyclopropyl)ethyl)amino)butyl)carbamate

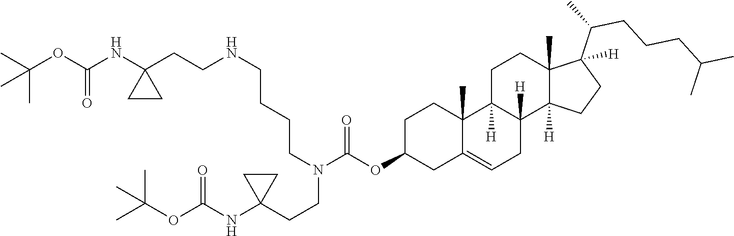

To a solution of di-tert-butyl (((butane-1,4-diylbis(azanediyl))bis(ethane-2,1-diyl))bis(cyclopropane-1,1-diyl))dicarbamate (0.47 g, 1.03 mmol) in dry toluene (20 mL) set stirring under nitrogen was added triethylamine (0.43 mL, 3.08 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.57 g, 1.03 mmol) was added, and the solution was heated to 90° C. and allowed to proceed overnight. The following morning, the reaction mixture was allowed to cool to room temperature, diluted with toluene, washed with water (3×15 mL), dried over sodium sulfate, filtered, and concentrated to an oil. The oil was taken up in DCM and purified via silica gel chromatography in DCM with a 0-70% (70:25:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-((tert-butoxycarbonyl)amino)cyclopropyl)ethyl)(4-((2-(1-((tert-butoxycarbonyl)amino)cyclopropyl)ethyl)amino)butyl)carbamate as a colorless oil (0.35 g, 0.41 mmol, 39.4%). UPLC/ELSD: RT=2.65 min. MS (ES): m/z (MH⁺) 868.3 for C₅₂H₉₀N₄O₆. ¹H NMR (300 MHz, CDCl₃) δ: ppm 5.80 (m, 1H), 5.40 (m, 1H), 4.96 (br. m, 2H), 4.50 (m, 1H), 3.42 (m, 2H), 3.15 (m, 2H), 2.74 (t, 2H), 2.61 (m, 2H), 2.34 (m, 2H), 2.00 (m, 5H), 1.70 (m, 4H), 1.56 (br. m, 8H), 1.44 (s, 18H), 1.15 (br. m, 10H), 1.03 (s, 5H), 0.94 (d, 4H, J=6 Hz), 0.89 (d, 5H, J=6 Hz), 0.69 (br. m, 11H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-aminocyclopropyl)ethyl)(4-((2-(1-aminocyclopropyl)ethyl)amino)butyl)carbamate trihydrochloride

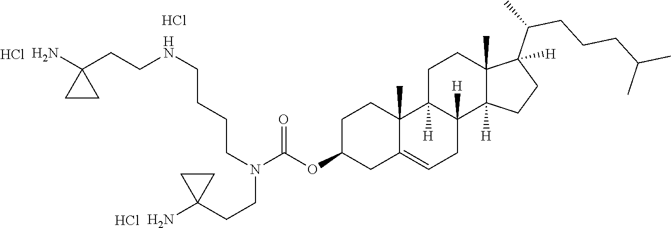

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-((tert-butoxycarbonyl)amino)cyclopropyl)ethyl)(4-((2-(1-((tert-butoxycarbonyl)amino)cyclopropyl)ethyl)amino)butyl)carbamate (0.35 g, 0.41 mmol) in isopropanol (10 mL) set stirring under nitrogen was added hydrochloric acid (5 N in isopropanol, 0.81 mL, 4.05 mmol) dropwise. The solution was heated to 40° C. and allowed to proceed overnight. The following morning, the mixture was cooled to room temperature, and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-aminocyclopropyl)ethyl)(4-((2-(1-aminocyclopropyl)ethyl)amino)butyl)carbamate trihydrochloride as a white solid (0.27 g, 0.32 mmol, 80.2%). UPLC/ELSD: RT=1.60 min. MS (ES): m/z (MH⁺) 668.7 for C₄₂H₇₇Cl₃N₄O₂. ¹H NMR (300 MHz, MeOD) δ: ppm 5.41 (m, 1H), 4.46 (br. m, 1H), 3.93 (br. m, 1H), 3.53 (m, 2H), 3.33 (m, 6H), 3.11 (m, 2H), 2.40 (m, 2H), 2.15 (br. m, 4H), 1.93 (br. m, 5H), 1.55 (br. m, 15H), 1.18 (br. m, 11H), 1.08 (m, 8H), 0.97 (m, 7H), 0.89 (m, 7H), 0.74 (s, 3H).

### BP. Compound SA125: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride

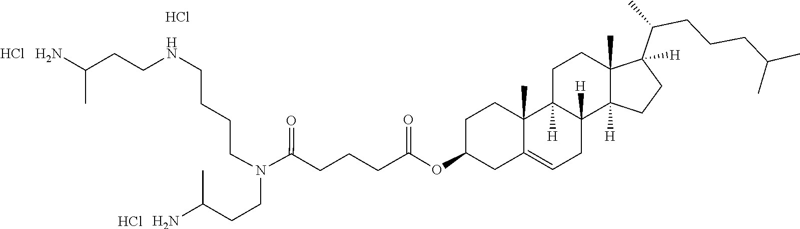

### Step 1: (3S,8S,9S,10R,13R,14S,17R)-10,13-dimethyl-7-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate

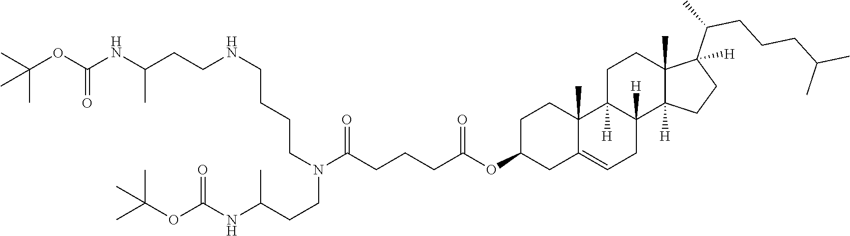

To a solution of 5-{[(1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl]oxy}-5-oxopentanoic acid (0.30 g, 0.59 mmol) in dry DCM (15 mL) stirring under nitrogen was added di-tert-butyl ((butane-1,4-diylbis(azanediyl))bis(butane-4,2-diyl))dicarbamate (0.77 g, 1.78 mmol), dimethylaminopyridine (0.15 g, 1.19 mmol), and 1-ethyl-3-(3-dimethylaminopropyl)carbodiimide hydrochloride (0.23 g, 1.19 mmol). The mixture was allowed to stir at room temperature and proceed overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate as a light yellow oil (0.18 g, 0.20 mmol, 32.9%). UPLC/ELSD: RT: 2.50 min. MS (ES): m/z (MH⁺) 914.4 for C₅₄H₉₆N₄O₇. ¹H NMR (300 MHz, CDCl₃) δ: ppm 5.33 (m, 1H), 4.82 (br. m, 3H), 3.62 (br. m, 2H), 3.24 (m, 4H), 2.55 (m, 4H), 2.31 (m, 6H), 1.89 (m, 7H), 1.54 (m, 12H), 1.39 (s, 20H), 1.28 (m, 6H), 1.11 (d, 12H, J=6 Hz), 0.97 (s, 6H), 0.87 (d, 4H, J=6 Hz), 0.82 (d, 6H, J=6 Hz), 0.63 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride

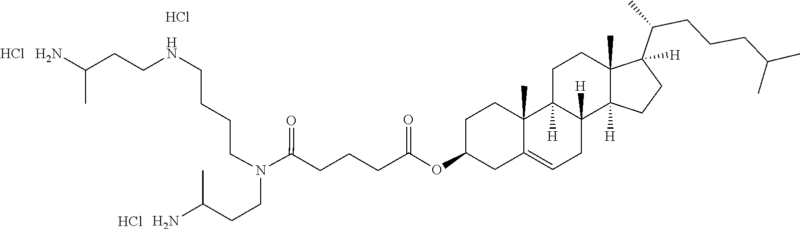

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate (0.18 g, 0.20 mmol) in isopropanol (5 mL) set stirring under nitrogen was added hydrochloric acid (5 N in isopropanol, 0.39 mL, 1.95 mmol) dropwise. The solution was heated to 40° C. and allowed to proceed overnight. The following morning, the mixture was cooled to room temperature and dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride as a white solid (0.07 g, 0.08 mmol, 40.1%). UPLC/ELSD: RT=1.60 min. MS (ES): m/z (MH⁺) 668.7 for C₄₂H₇₇Cl₃N₄O₂. ¹H NMR (300 MHz, MeOD) δ: ppm 5.41 (m, 1H), 4.46 (br. m, 1H), 3.93 (br. m, 1H), 3.53 (m, 2H), 3.33 (m, 6H), 3.11 (m, 2H), 2.40 (m, 2H), 2.15 (br. m, 4H), 1.93 (br. m, 5H), 1.55 (br. m, 15H), 1.18 (br. m, 11H), 1.08 (m, 8H), 0.97 (m, 7H), 0.89 (m, 7H), 0.74 (s, 3H).

### BQ. Compound SA126: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminopentan-2-yl)(4-((4-aminopentan-2-yl)amino)butyl)carbamate trihydrochloride

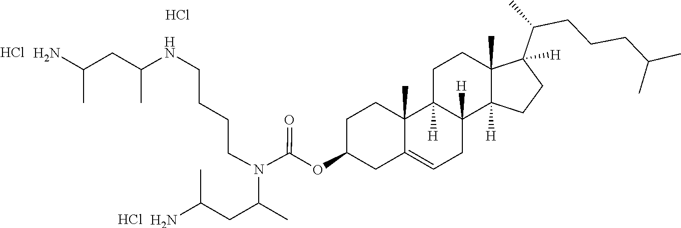

### Step 1: tert-butyl (4-((2-nitrophenyl)sulfonamido)pentan-2-yl)carbamate

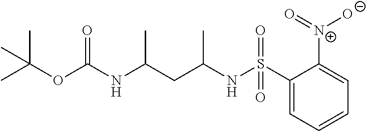 [SMILES: CC(CC(C)NS(=O)(=O)c1ccccc1[N+](=O)[O-])NC(=O)OC(C)(C)C]

To a solution of tert-butyl (4-aminopentan-2-yl)carbamate (2.50 g, 11.74 mmol) in dry DCM (50 mL) set stirring under nitrogen was added triethylamine (3.27 mL, 23.48 mmol). The solution was cooled to 0° C., and then a solution of 2-nitrobenzenesulfonyl chloride (2.86 g, 12.91 mmol) in 50 mL dry DCM was added dropwise over 30 minutes. The reaction was allowed to proceed at 0° C. for an hour and then at room temperature for an additional three hours. The mixture was then diluted with an additional 10 mL DCM, washed with saturated aqueous sodium bicarbonate (1×100 mL), water (1×100 mL), 10% aqueous citric acid (1×100 mL), water (1×100 mL), and brine (1×100 mL), dried over sodium sulfate, filtered, and concentrated to give tert-butyl (4-((2-nitrophenyl)sulfonamido)pentan-2-yl)carbamate as a white solid (4.56 g, 11.77 mmol, quantitative). UPLC/ELSD: RT=0.78 min. MS (ES): m/z (MH⁺) 388.4 for C₁₆H₂₅N₃O₆S. ¹H NMR (300 MHz, CDCl₃) δ: ppm 8.10 (m, 1H), 7.79 (m, 1H), 7.66 (m, 2H), 5.31 (br. s, 1H), 4.29 (br. s, 1H), 3.59 (m, 2H), 1.64 (m, 2H), 1.38 (s, 9H), 1.05 (t, 6H).

### Step 2: di-tert-butyl ((butane-1,4-diylbis(azanediyl))bis(pentane-4,2-diyl))dicarbamate

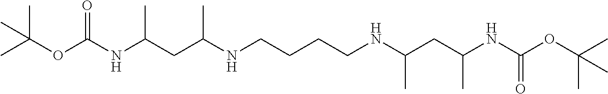 [SMILES: CC(CC(C)NC(=O)OC(C)(C)C)NCCCCNC(C)CC(C)NC(=O)OC(C)(C)C]

To a solution of tert-butyl (4-((2-nitrophenyl)sulfonamido)pentan-2-yl)carbamate (4.56 g, 11.77 mmol) in dry DMF (50 mL) set stirring under nitrogen was added potassium carbonate (4.72 g, 34.18 mmol) and 1,4-diiodobutane (0.74 mL, 5.60 mmol). The solution was heated to 40° C. and allowed to proceed overnight. The following morning, benzyl bromide (0.55 mL, 4.65 mmol) was added and the reaction was allowed to proceed at room temperature for 8 h. Then, thiophenol (2.21 mL, 21.57 mmol), potassium carbonate (2.32 g, 16.81 mmol), and an additional 20 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×10 mL), dried over potassium carbonate, filtered, and concentrated to an oil. The oil was taken up again in DCM and purified via silica gel chromatography in DCM with a 0-50% (50:45:5 DCM/MeOH/concentrated aqueous ammonium hydroxide) gradient. Fractions containing product were combined and concentrated to give di-tert-butyl ((butane-1,4-diylbis(azanediyl))bis(pentane-4,2-diyl))dicarbamate as a colorless oil (2.07 g, 4.51 mmol, 80.5%). UPLC/ELSD: RT=0.27 min. MS (ES): m/z (MH⁺) 459.6 for C₂₄H₅₀N₄O₄. ¹H NMR (300 MHz, CDCl₃) δ: ppm 5.31 (m, 2H), 3.77 (m, 2H), 2.75 (m, 4H), 2.51 (m, 2H), 1.56 (m, 7H), 1.45 (s, 20H), 1.17 (m, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)pentan-2-yl)(4-((4-((tert-butoxycarbonyl)amino)pentan-2-yl)amino)butyl)carbamate

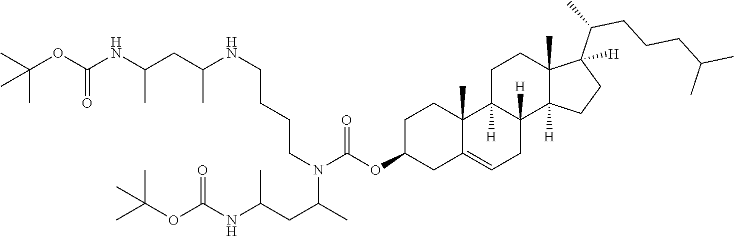

To a solution of di-tert-butyl ((butane-1,4-diylbis(azanediyl))bis(pentane-4,2-diyl))dicarbamate (0.83 g, 1.80 mmol) in dry toluene (20 mL) set stirring under nitrogen was added triethylamine (0.76 mL, 5.40 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.99 g, 1.80 mmol) was added, and the solution was heated to 90° C. and allowed to proceed overnight. The following morning, the reaction mixture was allowed to cool to room temperature, diluted with toluene, and washed with water (3×15 mL), dried over sodium sulfate, filtered, and concentrated to an oil. The oil was taken up in DCM and purified via silica gel chromatography in DCM with a 0-30% (50:45:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)pentan-2-yl)(4-((4-((tert-butoxycarbonyl)amino)pentan-2-yl)amino)butyl)carbamate as a colorless oil (0.70 g, 0.80 mmol, 44.4%). UPLC/ELSD: RT=2.74 min. MS (ES): m/z (MH⁺) 872.3 for C₅₂H₉₄N₄O₆. ¹H NMR (300 MHz, CDCl₃) δ: ppm 5.26 (m, 1H), 4.61 (m, 1H), 4.41 (br. m, 1H), 3.69 (br. m, 4H), 2.97 (m, 2H), 2.58 (m, 2H), 2.24 (br. m, 4H), 1.89 (m, 6H), 1.44 (m, 11H), 1.33 (s, 20H), 1.24 (br. m, 5H), 1.04 (m, 19H), 0.92 (s, 5H), 0.83 (d, 4H, J=6 Hz), 0.77 (d, 6H, J=6 Hz), 0.58 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminopentan-2-yl)(4-((4-aminopentan-2-yl)amino)butyl)carbamate trihydrochloride

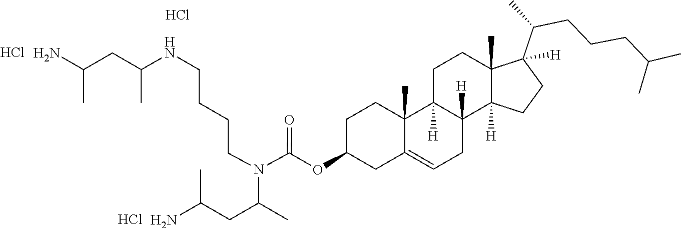

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)pentan-2-yl)(4-((4-((tert-butoxycarbonyl)amino)pentan-2-yl)amino)butyl)carbamate (0.70 g, 0.80 mmol) in isopropanol (10 mL) set stirring under nitrogen was added hydrochloric acid (5 N in isopropanol, 1.60 mL, 7.99 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminopentan-2-yl)(4-((4-aminopentan-2-yl)amino)butyl)carbamate trihydrochloride as a white solid (0.46 g, 0.57 mmol, 70.7%). UPLC/ELSD: RT=1.96 min. MS (ES): m/z (MH⁺) 672.1 for C₄₂H₈₁Cl₃N₄O₂. ¹H NMR (300 MHz, MeOD) δ: ppm 5.42 (m, 1H), 4.47 (br. m, 1H), 4.28 (m, 1H), 3.53 (br. m, 2H), 3.53 (m, 2H), 3.33 (s, 2H), 3.15 (m, 4H), 2.40 (m, 2H), 1.93 (br. m, 18H), 1.42 (br. m, 12H), 1.28 (br. m, 4H), 1.16 (d, 8H, J=6 Hz), 1.08 (m, 6H), 0.98 (d, 4H, J=9 Hz), 0.89 (d, 6H, J=6 Hz), 0.74 (s, 3H).

### BR. Compound SA127: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopentyl)(4-((3-aminopentyl)amino)butyl)carbamate trihydrochloride

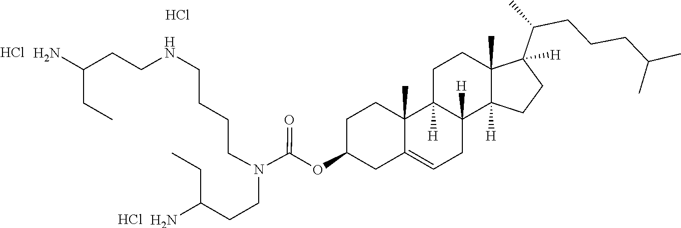

### Step 1: tert-butyl (1-((2-nitrophenyl)sulfonamido)pentan-3-yl)carbamate

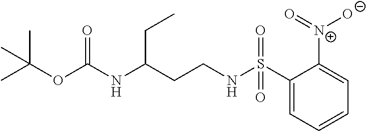 [SMILES: CCC(CCNS(=O)(=O)c1ccccc1[N+](=O)[O-])NC(=O)OC(C)(C)C]

To a solution of tert-butyl (1-aminopentan-3-yl)carbamate (2.00 g, 9.39 mmol) in dry DCM (50 mL) set stirring under nitrogen was added triethylamine (2.62 mL, 18.78 mmol). The solution was cooled to 0° C. and then a solution of 2-nitrobenzenesulfonyl chloride (2.29 g, 10.33 mmol) in 50 mL dry DCM was added dropwise over 30 minutes. The reaction was allowed to proceed at 0° C. for an hour and then at room temperature for an additional three hours. The mixture was then diluted with an additional 10 mL DCM, washed with saturated aqueous sodium bicarbonate (1×100 mL), water (1×100 mL), 10% aqueous citric acid (1×100 mL), water (1×100 mL), and brine (1×100 mL), dried over sodium sulfate, filtered, and concentrated to give tert-butyl (1-((2-nitrophenyl)sulfonamido)pentan-3-yl)carbamate as a white solid (3.73 g, 9.61 mmol, quantitative). UPLC/ELSD: RT=0.81 min. MS (ES): m/z (MH⁺) 388.4 for C₁₆H₂₅N₃O₆S. 1H NMR (300 MHz, CDCl₃) δ: ppm 8.12 (m, 1H), 7.83 (m, 1H), 7.74 (m, 2H), 6.26 (br. s, 1H), 4.27 (br. s, 1H), 3.54 (m, 1H), 3.31 (m, 1H), 3.03 (m, 1H), 1.76 (m, 1H), 1.41 (s, 9H), 0.87 (t, 3H).

### Step 2: di-tert-butyl ((butane-1,4-diylbis(azanediyl))bis(pentane-1,3-diyl))dicarbamate

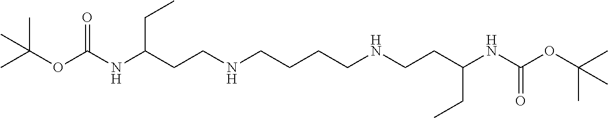 [SMILES: CCC(CCNCCCCNCCC(CC)NC(=O)OC(C)(C)C)NC(=O)OC(C)(C)C]

To a solution of tert-butyl (1-((2-nitrophenyl)sulfonamido)pentan-3-yl)carbamate (3.73 g, 9.61 mmol) in dry DMF (50 mL) set stirring under nitrogen was added potassium carbonate (3.86 g, 27.93 mmol) and 1,4-diiodobutane (0.60 mL, 4.58 mmol). The solution was heated to 40° C. and allowed to proceed overnight. The following morning, benzyl bromide (0.45 mL, 3.80 mmol) was added, and the reaction was allowed to proceed at room temperature for 8 h. Then thiophenol (1.80 mL, 17.63 mmol), potassium carbonate (1.90 g, 13.73 mmol), and an additional 20 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×10 mL), dried over potassium carbonate, filtered, and concentrated to an oil. The oil was taken up again in DCM and purified via silica gel chromatography in DCM with a 0-50% (50:45:5 DCM/MeOH/concentrated aqueous ammonium hydroxide) gradient. Fractions containing product were combined and concentrated to give di-tert-butyl ((butane-1,4-diylbis(azanediyl))bis(pentane-1,3-diyl))dicarbamate as a colorless oil (1.40 g, 3.05 mmol, 66.7%). UPLC/ELSD: RT=0.34 min. MS (ES): m/z (MH⁺) 459.6 for C₂₄H₅₀N₄O₄. ¹H NMR (300 MHz, CDCl₃) δ: ppm 4.94 (m, 2H), 3.29 (m, 4H), 2.47 (m, 8H), 1.53 (m, 2H), 1.36 (m, 8H), 1.21 (s, 19H), 0.69 (t, 6H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)pentyl)(4-((3-((tert-butoxycarbonyl)amino)pentyl)amino)butyl)carbamate

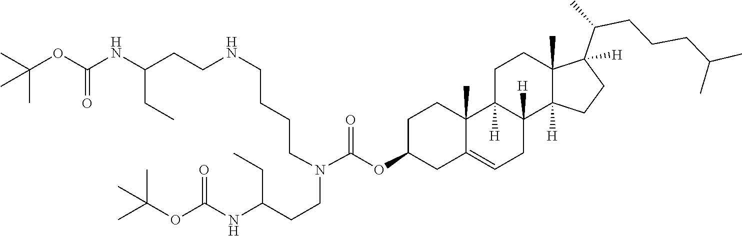

To a solution of di-tert-butyl ((butane-1,4-diylbis(azanediyl))bis(pentane-1,3-diyl))dicarbamate (0.58 g, 1.27 mmol) in dry toluene (20 mL) set stirring under nitrogen was added triethylamine (0.54 mL, 3.82 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.70 g, 1.27 mmol) was added, and the solution was heated to 90° C. and allowed to proceed overnight. The following morning, the reaction mixture was allowed to cool to room temperature, diluted with toluene, washed with water (3×15 mL), dried over sodium sulfate, filtered, and concentrated to an oil. The oil was taken up in DCM and purified via silica gel chromatography in DCM with a 0-30% (50:45:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)pentyl)(4-((3-((tert-butoxycarbonyl)amino)pentyl)amino)butyl)carbamate as a colorless oil (0.67 g, 0.77 mmol, 60.5%). UPLC/ELSD: RT=3.06 min. MS (ES): m/z (MH⁺) 872.3 for C₅₂H₉₄N₄O₆. ¹H NMR (300 MHz, CDCl₃) δ: ppm 5.24 (m, 1H), 4.61 (br. m, 3H), 3.43 (br. m, 2H), 3.11 (br. m, 4H), 2.47 (m, 4H), 2.22 (m, 2H), 1.87 (br. m, 8H), 1.42 (m, 13H), 1.32 (s, 24H), 1.14 (br. m, 13H), 1.04 (m, 19H), 0.91 (s, 6H), 0.79 (d, 9H, J=6 Hz), 0.76 (d, 7H, J=6 Hz), 0.56 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopentyl)(4-((3-aminopentyl)amino)butyl)carbamate trihydrochloride

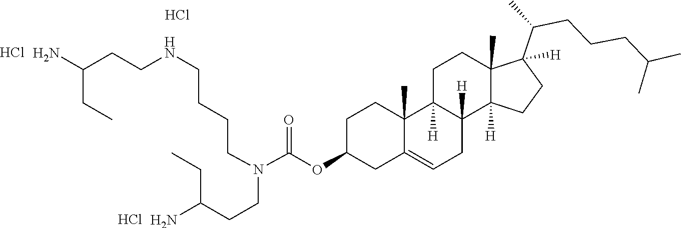

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)pentyl)(4-((3-((tert-butoxycarbonyl)amino)pentyl)amino)butyl)carbamate (0.67 g, 0.77 mmol) in isopropanol (10 mL) set stirring under nitrogen was added hydrochloric acid (5 N in isopropanol, 1.54 mL, 7.70 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopentyl)(4-((3-aminopentyl)amino)butyl)carbamate trihydrochloride as a white solid (0.52 g, 0.65 mmol, 84.4%). UPLC/ELSD: RT=1.90 min. MS (ES): m/z (MH⁺) 672.1 for C₄₂H₈₁Cl₃N₄O₂. ¹H NMR (300 MHz, MeOD) δ: ppm 5.31 (m, 1H), 4.34 (br. m, 1H), 3.81 (m, 2H), 3.22 (br. m, 6H), 3.01 (m, 5H), 2.26 (m, 2H), 1.98 (m, 2H), 1.94 (s, 4H), 1.81 (br. m, 5H), 1.63 (br. m, 8H), 1.44 (br. m, 7H), 1.28 (br. m, 5H), 1.06 (d, 15H, J=9 Hz), 0.96 (m, 11H), 0.84 (d, 4H, J=6 Hz), 0.80 (d, 6H, J=6 Hz), 0.63 (s, 3H).

### BS. Compound SA128: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(aminomethyl)cyclopropyl)methyl)(4-(((1-(aminomethyl)cyclopropyl)methyl)amino)butyl)carbamate trihydrochloride

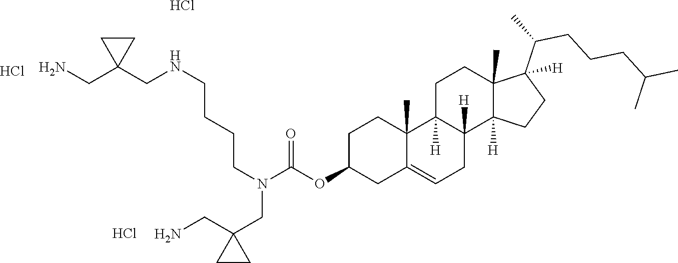

### Step 1: tert-butyl ((1-(((2-nitrophenyl)sulfonamido)methyl)cyclopropyl)methyl)carbamate

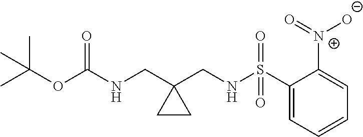 [SMILES: CC(C)(C)OC(=O)NCC1(CNS(=O)(=O)c2ccccc2[N+](=O)[O-])CC1]

To a solution of tert-butyl ((1-(aminomethyl)cyclopropyl)methyl)carbamate (2.50 g, 11.86 mmol) in dry DCM (25 mL) set stirring under nitrogen was added triethylamine (3.31 mL, 23.72 mmol). The solution was cooled to 0° C. and then a solution of 2-nitrobenzenesulfonyl chloride (2.89 g, 13.04 mmol) in 50 mL dry DCM was added dropwise over 30 minutes. The reaction was allowed to proceed at 0° C. for an hour, and then at room temperature for an additional three hours. Following, the mixture was diluted with an additional 10 mL DCM, washed with saturated aqueous sodium bicarbonate (1×100 mL), water (1×100 mL), 10% aqueous citric acid (1×100 mL), water (1×100 mL), and brine (1×100 mL), dried over sodium sulfate, filtered, and concentrated to give tert-butyl ((1-(((2-nitrophenyl)sulfonamido)methyl)cyclopropyl)methyl)carbamate as a white solid (4.60 g, 11.92 mmol, quantitative). UPLC/ELSD: RT=0.83 min. MS (ES): m/z (MH⁺) 386.4 for C₁₆H₂₃N₃O₆S. ¹H NMR (300 MHz, CDCl₃) δ: ppm 8.11 (m, 1H), 7.85 (m, 1H), 7.75 (m, 2H), 6.33 (br. s, 1H), 4.82 (br. s, 1H), 3.08 (d, 2H, J=6 Hz), 3.02 (d, 2H, J=6 Hz), 1.45 (s, 9H), 0.48 (m, 4H).

### Step 2: di-tert-butyl ((((butane-1,4-diylbis(azanediyl))bis(methylene))bis(cyclopropane-1,1-diyl))bis(methylene))dicarbamate

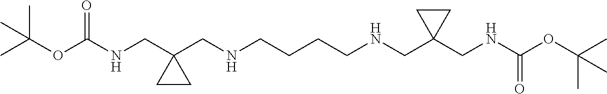 [SMILES: CC(C)(C)OC(=O)NCC1(CNCCCCNCC2(CNC(=O)OC(C)(C)C)CC2)CC1]

To a solution of tert-butyl ((1-(((2-nitrophenyl)sulfonamido)methyl)cyclopropyl)methyl)carbamate (4.60 g, 11.92 mmol) in dry DMF (50 mL) set stirring under nitrogen was added potassium carbonate (4.79 g, 34.64 mmol) and 1,4-diiodobutane (0.75 mL, 5.68 mmol). The solution was heated to 40° C. and allowed to proceed overnight. The following morning, benzyl bromide (0.56 mL, 4.71 mmol) was added, and the reaction was allowed to proceed at room temperature for 8 h. Then thiophenol (2.24 mL, 21.86 mmol), potassium carbonate (2.35 g, 17.03 mmol), and an additional 20 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×10 mL), dried over potassium carbonate, filtered, and concentrated to an oil. The oil was taken up again in DCM and purified via silica gel chromatography in DCM with a 0-50% (50:45:5 DCM/MeOH/concentrated aqueous ammonium hydroxide) gradient. Fractions containing product were combined and concentrated to give di-tert-butyl ((((butane-1,4-diylbis(azanediyl))bis(methylene))bis(cyclopropane-1,1-diyl))bis(methylene))dicarbamate as a colorless oil (2.47 g, 5.43 mmol, 95.6%). UPLC/ELSD: RT=0.28 min. MS (ES): m/z (MH⁺) 455.6 for C₂₄H₄₆N₄O₄. ¹H NMR (300 MHz, CDCl₃) δ: ppm 5.67 (m, 2H), 2.87 (d, 4H, J=6 Hz), 2.41 (m, 4H), 2.34 (s, 4H), 1.36 (m, 5H), 1.28 (s, 19H), 0.25 (m, 4H), 0.17 (m, 4H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(((tert-butoxycarbonyl)amino)methyl)cyclopropyl)methyl)(4-(((1-(((tert-butoxycarbonyl)amino)methyl)cyclopropyl)methyl)amino)butyl)carbamate

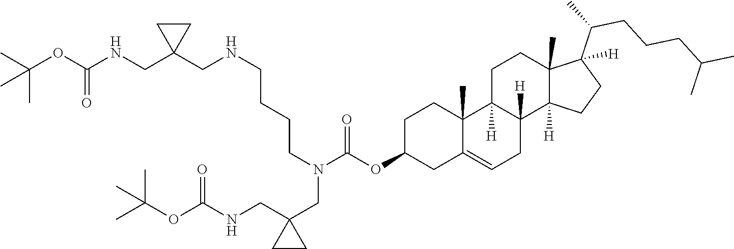

To a solution of di-tert-butyl ((((butane-1,4-diylbis(azanediyl))bis(methylene))bis(cyclopropane-1,1-diyl))bis(methylene))dicarbamate (0.92 g, 2.02 mmol) in dry toluene (20 mL) set stirring under nitrogen was added triethylamine (0.85 mL, 6.04 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (1.11 g, 2.02 mmol) was added. The solution was heated to 90° C. and allowed to proceed overnight. The following morning, the reaction mixture was allowed to cool to room temperature, diluted with toluene, and washed with water (3×15 mL), dried over sodium sulfate, filtered, and concentrated to an oil. The oil was taken up in DCM and purified via silica gel chromatography in DCM with a 0-30% (50:45:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(((tert-butoxycarbonyl)amino)methyl)cyclopropyl)methyl)(4-(((1-(((tert-butoxycarbonyl)amino)methyl)cyclopropyl)methyl)amino)butyl)carbamate as a colorless oil (0.83 g, 0.95 mmol, 47.3%). UPLC/ELSD: RT=3.05 min. MS (ES): m/z (MH⁺) 868.3 for C₅₂H₉₀N₄O₆. ¹H NMR (300 MHz, CDCl₃) δ: ppm 5.77 (br. m, 1H), 5.29 (m, 1H), 4.44 (br. m, 1H), 3.13 (br. m, 4H), 2.97 (m, 2H), 2.85 (m, 2H), 2.50 (t, 2H), 2.42 (s, 2H), 2.26 (br. m, 2H), 1.85 (br. m, 5H), 1.47 (m, 9H), 1.34 (s, 19H), 1.05 (br. m, 11H), 0.94 (s, 6H), 0.84 (d, 4H, J=6 Hz), 0.78 (d, 6H, J=6 Hz), 0.59 (s, 3H), 0.50 (m, 2H), 0.35 (m, 2H), 0.26 (m, 4H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(aminomethyl)cyclopropyl)methyl)(4-(((1-(aminomethyl)cyclopropyl)methyl)amino)butyl)carbamate trihydrochloride

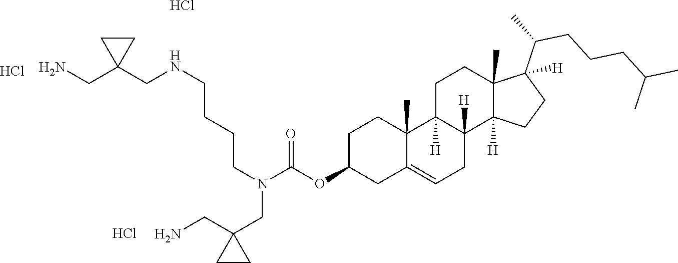

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(((tert-butoxycarbonyl)amino)methyl)cyclopropyl)methyl)(4-(((1-(((tert-butoxycarbonyl)amino)methyl)cyclopropyl)methyl)amino)butyl)carbamate (0.83 g, 0.95 mmol) in isopropanol (10 mL) set stirring under nitrogen was added hydrochloric acid (5 N in isopropanol, 1.91 mL, 9.54 mmol) dropwise. The solution was heated to 40° C. and allowed to proceed overnight. The following morning, the mixture was cooled to room temperature, and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(aminomethyl)cyclopropyl)methyl)(4-(((1-(aminomethyl)cyclopropyl)methyl)amino)butyl)carbamate trihydrochloride as a white solid (0.67 g, 0.83 mmol, 86.8%). UPLC/ELSD: RT=1.61 min. MS (ES): m/z (MH⁺) 668.1 for C₄₂H₇₇Cl₃N₄O₂. ¹H NMR (300 MHz, MeOD) δ: ppm 5.44 (m, 1H), 4.54 (br. m, 1H), 3.86 (m, 2H), 3.33 (br. m, 6H), 3.15 (m, 6H), 2.81 (m, 2H), 2.44 (m, 2H), 1.73 (br. m, 11H), 1.55 (br. m, 6H), 1.39 (m, 5H), 1.18 (d, 17H, J=6 Hz), 1.09 (s, 6H), 0.98 (d, 7H, J=9 Hz), 0.90 (d, 9H, J=6 Hz), 0.74 (br. m, 7H).

### BT. Compound SA129: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N—((S)-2,5-diaminopentanoyl)glycinate trihydrochloride

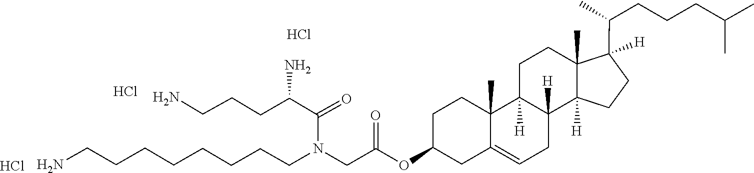

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N—((S)-2,5-bis((tert-butoxycarbonyl)amino)pentanoyl)-N-(8-((tert-butoxycarbonyl)amino)octyl)glycinate

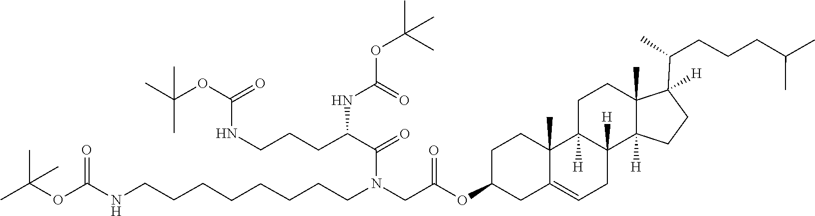

A solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.250 g, 0.373 mmol) and (2S)-2,5-bis[(tert-butoxycarbonyl)amino]pentanoic acid (0.161 g, 0.484 mmol) in DCM (3.75 mL) was cooled to 0° C. in an ice bath. Then 1-ethyl-3-(3-dimethylaminopropyl)carbodiimide hydrochloride (0.107 g, 0.559 mmol) was added. The reaction mixture stirred at rt and was monitored by LCMS. At 17 h, the reaction mixture was cooled to 0° C. in an ice bath, and then water (3.0 mL) was added. The layers were separated, and the aqueous layer was extracted with DCM (10 mL). The combined organics were washed with 5% aq. NaHCO₃ solution, passed through a hydrophobic frit, dried over Na₂SO₄, and concentrated. The crude material was purified via silica gel chromatography (10-40% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N—((S)-2,5-bis((tert-butoxycarbonyl)amino)pentanoyl)-N-(8-((tert-butoxycarbonyl)amino)octyl)glycinate (0.380 g, quant.) as a clear oil. UPLC/ELSD: RT=4.03 min. MS (ES): m/z=987.54 [M+H]⁺ for C₅₇H₁₀₀N₄O₉. ¹H NMR (300 MHz, CDCl₃): δ 5.20-5.43 (m, 2H), 4.24-4.80 (m, 4H), 4.29 (d, 1H, J=17.0 Hz), 3.72 (d, 1H, J=17.2 Hz), 3.02-3.53 (m, 6H), 2.20-2.42 (m, 2H), 0.93-2.18 (br. m, 69H), 1.01 (s, 3H), 0.91 (d, 3H, J=6.5 Hz), 0.86 (d, 3H, J=6.6 Hz), 0.86 (d, 3H, J=6.6 Hz), 0.63-0.76 (m, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N—((S)-2,5-diaminopentanoyl)glycinate trihydrochloride

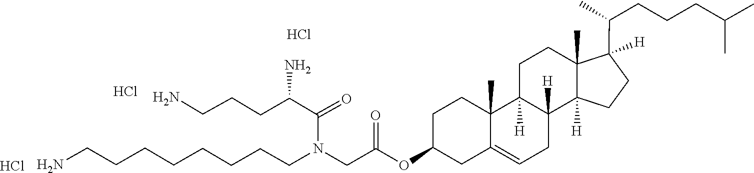

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N—((S)-2,5-bis((tert-butoxycarbonyl)amino)pentanoyl)-N-(8-((tert-butoxycarbonyl)amino)octyl)glycinate (0.356 g, 0.347 mmol) in iPrOH (3.5 mL) was added 5-6 N HCl in iPrOH (0.50 mL). The reaction mixture stirred at 40° C. and was monitored by LCMS. At 19 h, additional 5-6 N HCl in iPrOH (0.10 mL) was added. At 22 h, the reaction mixture was cooled to rt, and then ACN (7 mL) was added. The suspension was cooled in an ice bath, and then solids were collected by vacuum filtration rinsing with 2:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N—((S)-2,5-diaminopentanoyl)glycinate trihydrochloride (0.182 g, 0.203 mmol, 58.6%) as a white solid. UPLC/ELSD: RT=1.94 min. MS (ES): m/z=343.40 [M+2H]²⁺ for C₄₂H₇₆N₄O₃. ¹H NMR (300 MHz, DMSO): δ 7.85-8.59 (m, 9H), 5.24-5.55 (m, 1H), 4.25-4.68 (m, 2H), 4.17 (d, 1H, J=17.0 Hz), 3.95 (d, 1H, J=17.2 Hz), 3.12-3.61 (m, 2H), 2.65-2.85 (m, 4H), 2.20-2.42 (m, 2H), 0.92-2.03 (br. m, 42H), 0.98 (s, 3H), 0.89 (d, 3H, J=6.3 Hz), 0.84 (d, 3H, J=6.6 Hz), 0.84 (d, 3H, J=6.5 Hz), 0.65 (s, 3H).

### BU. Compound SA130: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(L-lysyl)-N-(8-aminooctyl)glycinate trihydrochloride

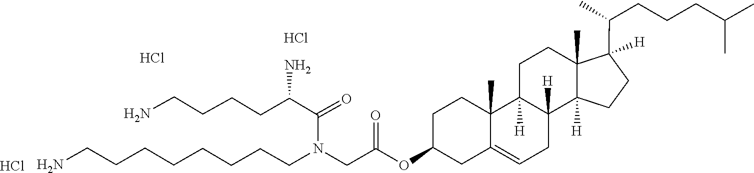

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N—(N₂,N₆-bis(tert-butoxycarbonyl)-L-lysyl)-N-(8-((tert-butoxycarbonyl)amino)octyl)glycinate

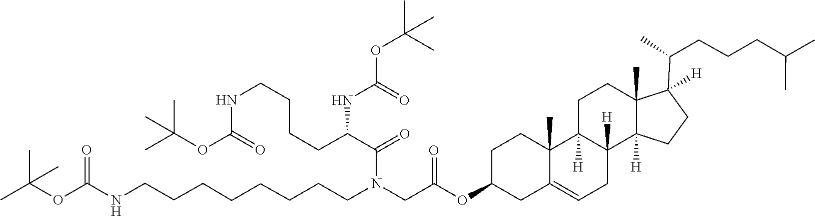

A solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.235 g, 0.350 mmol) and (2S)-2,6-bis[(tert-butoxycarbonyl)amino]hexanoic acid (0.121 g, 0.350 mmol) in DCM (3.5 mL) was cooled to 0° C. in an ice bath. Then 1-ethyl-3-(3-dimethylaminopropyl)carbodiimide hydrochloride (0.101 g, 0.525 mmol) was added. The reaction mixture stirred at rt and was monitored by LCMS. At 19 h, the reaction mixture was cooled to 0° C., and then additional (2S)-2,6-bis [(tert-butoxycarbonyl)aamino]hexanoic acid (23 mg) and 1-ethyl-3-(3-dimethylaminopropyl)carbodiimide hydrochloride (20 mg) were added. The reaction mixture stirred at rt. At 21 h, the reaction mixture was cooled to 0° C. in an ice bath, and then water (3.5 mL) was added. The layers were separated, and the aqueous layer was extracted with DCM (10 mL). The combined organics were washed with 5% aq. NaHCO₃ solution, passed through a hydrophobic frit, dried over Na₂SO₄, and concentrated. The crude material was purified via silica gel chromatography (10-40% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N—(N₂,N₆-bis(tert-butoxycarbonyl)-L-lysyl)-N-(8-((tert-butoxycarbonyl)amino)octyl)glycinate (0.323 g, 0.323 mmol, 92.3%) as a clear oil. UPLC/ELSD: RT=4.09 min. MS (ES): m/z=1001.35 [M+H]⁺ for C₅₈H₁₀₂N₄O₉. ¹H NMR (300 MHz, CDCl₃): δ 5.21-5.46 (m, 2H), 4.26-4.79 (m, 4H), 4.30 (d, 1H, J=17.1 Hz), 3.71 (d, 1H, J=17.0 Hz), 3.01-3.52 (m, 6H), 2.22-2.43 (m, 2H), 0.93-2.14 (br. m, 71H), 1.01 (s, 3H), 0.91 (d, 3H, J=6.4 Hz), 0.86 (d, 6H, J=6.6 Hz), 0.64-0.75 (m, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(L-lysyl)-N-(8-aminooctyl)glycinate trihydrochloride

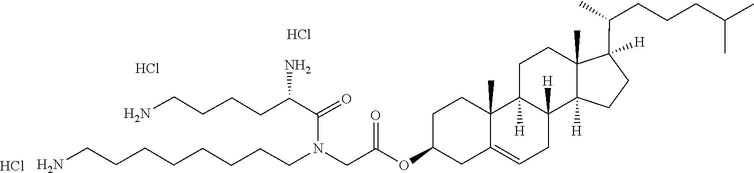

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N—(N₂,N₆-bis(tert-butoxycarbonyl)-L-lysyl)-N-(8-((tert-butoxycarbonyl)amino)octyl)glycinate (0.303 g, 0.303 mmol) in iPrOH (3.0 mL) was added 5-6 N HCl in iPrOH (0.45 mL). The reaction mixture stirred at 40° C. and was monitored by LCMS. At 19 h, additional 5-6 N HCl in iPrOH (0.10 mL) was added. At 22 h, the reaction mixture was cooled to rt, and then ACN (6 mL) was added. The suspension was cooled to 0° C. in an ice bath, and then solids were collected by vacuum filtration rinsing with 2:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(L-lysyl)-N-(8-aminooctyl)glycinate trihydrochloride (0.163 g, 0.173 mmol, 57.0%) as a white solid. UPLC/ELSD: RT =1.96 min. MS (ES): m/z=350.43 [M+2H]²⁺ for C₄₃H₇₈N₄O₃. ¹H NMR (300 MHz, DMSO): δ 7.89-8.51 (m, 9H), 5.22-5.41 (m, 1H), 4.23-4.61 (m, 2H), 4.19 (d, 1H, J=17.0 Hz), 3.93 (d, 1H, J=17.1 Hz), 3.13-3.51 (m, 2H), 2.64-2.85 (m, 4H), 2.22-2.36 (m, 2H), 0.92-2.04 (br. m, 44H), 0.98 (s, 3H), 0.90 (d, 3H, J=6.3 Hz), 0.84 (d, 6H, J=6.6 Hz), 0.65 (s, 3H).

### BV. Compound SA131: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)-L-lysinate trihydrochloride

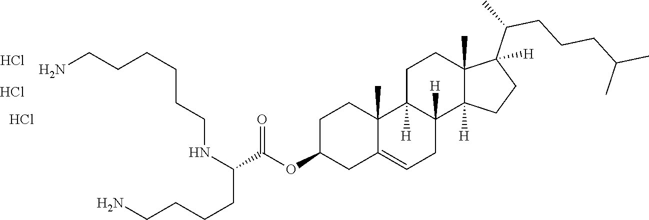

### Step 1: Methyl N₆-(tert-butoxycarbonyl)-N₂-((2-nitrophenyl)sulfonyl)-L-lysinate

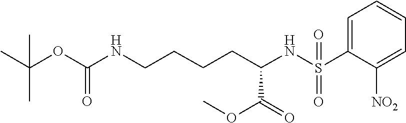 [SMILES: COC(=O)[C@H](CCCCNC(=O)OC(C)(C)C)NS(=O)(=O)c1ccccc1[N+](=O)[O-]]

A mixture of methyl (2S)-2-amino-6-[(tert-butoxycarbonyl)amino]hexanoate hydrochloride (1.000 g, 3.369 mmol), DMAP (cat.), and triethylamine (1.40 mL, 9.96 mmol) in DCM (15 mL) was cooled to 0° C., and then 2-nitrobenzenesulfonyl chloride (0.896 g, 4.04 mmol) in DCM (5 ml) was added dropwise.

The reaction mixture stirred at rt and was monitored by LCMS. At 1 h, the reaction mixture was cooled to 0° C., and then water (20 mL) was added. The layers were separated, and the organic layer was washed with 5% aq. NaHCO₃ solution, dried over Na₂SO₄, and concentrated. The crude material was purified via silica gel chromatography (30-70% EtOAc in hexanes) to afford methyl N₆-(tert-butoxycarbonyl)-N₂-((2-nitrophenyl)sulfonyl)-L-lysinate (1.44 g, 3.23 mmol, 95.9%) as a viscous, yellow oil. UPLC/ELSD: RT=0.78 min. MS (ES): m/z=390.30 [(M+H)—((CH₃)₂C═CH₂)]⁴ for C₁₈H₂₇N₃O₈S. ¹H NMR (300 MHz, CDCl₃): δ 8.02-8.10 (m, 1H), 7.89-7.97 (m, 1H), 7.68-7.78 (m, 2H), 6.08 (d, 1H, J=9.1 Hz), 4.53 (br. s, 1H), 4.16 (td, 1H, J=8.5, 5.0 Hz), 3.47 (s, 3H), 3.09 (td, 2H, J=6.1, 6.1 Hz), 1.33-1.94 (m, 6H), 1.44 (s, 9H).

### Step 2: Methyl N₆-(tert-butoxycarbonyl)-N₂-(6-((tert-butoxycarbonyl)amino)hexyl)-N₂-((2-nitrophenyl)sulfonyl)-L-lysinate

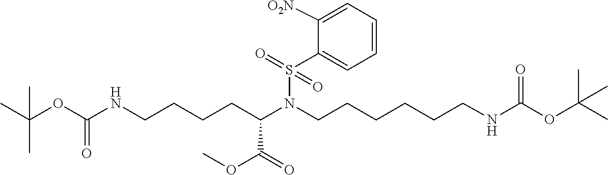 [SMILES: COC(=O)[C@H](CCCCNC(=O)OC(C)(C)C)N(CCCCCCNC(=O)OC(C)(C)C)S(=O)(=O)c1ccccc1[N+](=O)[O-]]

Methyl (2S)-6-[(tert-butoxycarbonyl)amino]-2-(2-nitrobenzenesulfonamido)hexanoate (0.603 g, 1.35 mmol), tert-butyl N-(6-bromohexyl)carbamate (0.504 g, 1.80 mmol), potassium carbonate (0.480 g, 3.48 mmol), and potassium iodide (0.046 g, 0.28 mmol) were combined in DMF (9.0 mL) and stirred at 80° C. Reaction was monitored by LCMS. At 18 h, the reaction mixture was cooled to rt, filtered, diluted with MTBE (100 mL), washed with water (3×) and brine, dried over Na₂SO₄, and concentrated. The crude material was purified via silica gel chromatography (20-70% EtOAc in hexanes) to afford methyl N₆-(tert-butoxycarbonyl)-N₂-(6-((tert-butoxycarbonyl)amino)hexyl)-N₂-((2-nitrophenyl)sulfonyl)-L-lysinate (0.693 g, 1.08 mmol, 79.4%) as a clear oil. UPLC/ELSD: RT=1.53 min. MS (ES): m/z=489.29 [(M+H)−2((CH₃)₂C═CH₂)—CO₂]⁺ for C₂₉H₄₈N₄O₁₀S. ¹H NMR (300 MHz, CDCl₃): δ 7.99-8.09 (m, 1H), 7.64-7.75 (m, 2H), 7.52-7.61 (m, 1H), 4.42-4.73 (m, 3H), 3.54 (s, 3H), 3.31-3.45 (m, 1H), 2.99-3.21 (m, 5H), 1.94-2.13 (m, 1H), 1.20-1.89 (br. m, 13H), 1.44 (s, 18H).

### Step 3: N₆-(tert-butoxycarbonyl)-N₂-(6-((tert-butoxycarbonyl)amino)hexyl)-N₂-((2-nitrophenyl)sulfonyl)-L-lysine

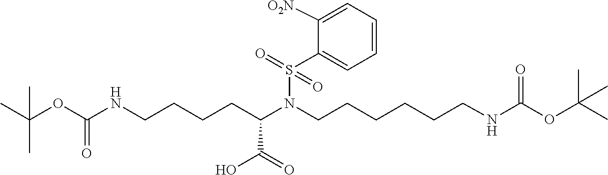 [SMILES: CC(C)(C)OC(=O)NCCCCCCN([C@@H](CCCCNC(=O)OC(C)(C)C)C(=O)O)S(=O)(=O)c1ccccc1[N+](=O)[O-]]

To a solution of methyl (2S)-6-[(tert-butoxycarbonyl)amino]-2-(N-{6-[(tert-butoxycarbonyl)amino]hexyl}-2-nitrobenzenesulfonamido)hexanoate (0.690 g, 1.07 mmol) in THF (7.0 mL) and MeOH (1.4 mL) was added aq. lithium hydroxide monohydrate (0.90 mL, 15 w/v %). The reaction mixture stirred at rt and was monitored by LCMS. At 19 h, the reaction mixture was concentrated to remove volatile organics, and then partitioned between water (50 mL) and EtOAc (50 mL). The biphasic mixture was washed with 5% aq. K₂CO₃ and 0.1 N aq. HCl, dried over Na₂SO₄, and concentrated to afford (2S)-6-[(tert-butoxycarbonyl)amino]-2-(N-{6-[(tert-butoxycarbonyl)amino]hexyl}-2-nitrobenzenesulfonamido)hexanoic acid (0.578 g, 0.916 mmol, 85.6%) as an amber oil. UPLC/ELSD: RT=1.27 min. MS (ES): m/z=475.35 [(M+H)—2((CH₃)₂C═CH₂)—CO₂]⁺ for C₂₈H₄₆N₄O₁₀S. ¹H NMR (300 MHz, CDCl₃): δ 8.03-8.12 (m, 1H), 7.63-7.74 (m, 2H), 7.53-7.62 (m, 1H), 4.48-4.79 (m, 3H), 2.94-3.42 (m, 6H), 1.92-2.18 (m, 1H), 1.20-1.83 (br. m, 13H), 1.44 (s, 18H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N₆-(tert-butoxycarbonyl)-N₂-(6-((tert-butoxycarbonyl)amino)hexyl)-N₂-((2-nitrophenyl)sulfonyl)-L-lysinate

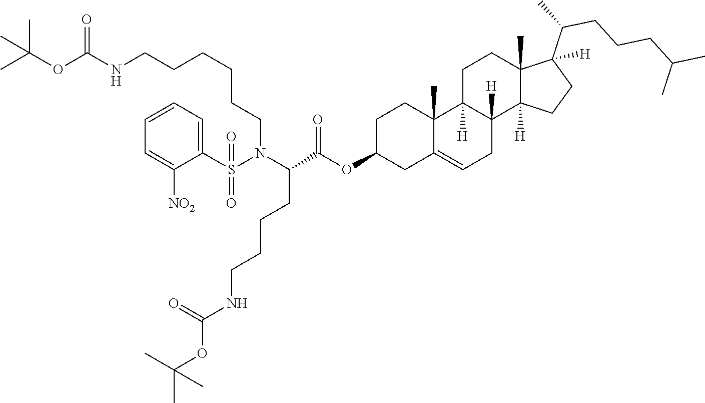

A mixture of (2S)-6-[(tert-butoxycarbonyl)amino]-2-(N-{6-[(tert-butoxycarbonyl)amino]hexyl}-2-nitrobenzenesulfonamido)hexanoic acid (0.560 g, 0.888 mmol), cholesterol (0.378 g, 0.977 mmol), and DMAP (cat.) in DCM (8.5 mL) was cooled to 0° C. Then 1-ethyl-3-(3-dimethylaminopropyl)carbodiimide hydrochloride (0.238 g, 1.24 mmol) was added. The reaction mixture slowly came to rt while stirring and was monitored by LCMS. At 19 h, the reaction mixture was cooled to 0° C., and then 5% aq. NaHCO₃ solution (8.5 mL) was added. Once the reaction mixture warmed to rt, the layers were separated. The aqueous layer was extracted with DCM (8 mL). The combined organics were passed through a hydrophobic frit, dried over Na₂SO₄, and concentrated. The crude material was purified via silica gel chromatography (0-40% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N₆-(tert-butoxycarbonyl)-N₂-(6-((tert-butoxycarbonyl)amino)hexyl)-N₂-((2-nitrophenyl)sulfonyl)-L-lysinate (0.562 g, 0.562 mmol, 63.3%) as a white foam. UPLC/ELSD: RT=3.62 min. MS (ES): m/z=900.19 [(M+H)—((CH₃)₂C═CH₂)—CO₂]⁺ for C₅₅H₉₀N₄O₁₀S. ¹H NMR (300 MHz, CDCl₃): δ 7.99-8.10 (m, 1H), 7.64-7.74 (m, 2H), 7.52-7.60 (m, 1H), 5.24-5.36 (m, 1H), 4.36-4.81 (m, 4H), 3.33-3.51 (m, 1H), 2.94-3.24 (m, 5H), 1.48 (br. m, 60H), 0.93 (s, 3H), 0.90 (d, 3H, J=6.4 Hz), 0.86 (d, 3H, J=6.6 Hz), 0.86 (d, 3H, J=6.6 Hz), 0.66 (s, 3H).

### Step 5: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N₆-(tert-butoxycarbonyl)-N₂-(6-((tert-butoxycarbonyl)amino)hexyl)-L-lysinate

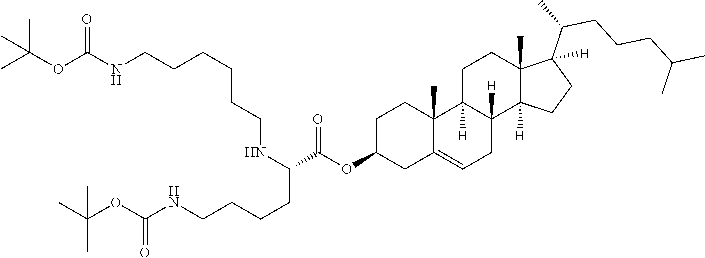

To a mixture of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N₆-(tert-butoxycarbonyl)-N₂-(6-((tert-butoxycarbonyl)amino)hexyl)-N₂-((2-nitrophenyl)sulfonyl)-L-lysinate (0.540 g, 0.540 mmol) and potassium carbonate (0.224 g, 1.621 mmol) in DMF (6.5 mL) was added thiophenol (0.10 mL, 0.980 mmol). The reaction mixture stirred at rt and was monitored by LCMS. At 17 h, the LCMS data was consistent with reaction completion. The reaction mixture was diluted with DCM (20 mL), and then filtered through a pad of Celite®. The filtrate was diluted to 80 mL with DCM, and then washed with water (3×) and 5% aq. NaHCO₃ solution. The organics were passed through a hydrophobic frit, dried over Na₂SO₄, and concentrated. The crude material was purified via silica gel chromatography (40-80% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N₆-(tert-butoxycarbonyl)-N₂-(6-((tert-butoxycarbonyl)amino)hexyl)-L-lysinate (0.400 g, 0.491 mmol, 90.9%) as a clear oil. UPLC/ELSD: RT=2.92 min. MS (ES): m/z=815.18 [M+H]⁺ for C₄₉H₈₇N₃O₆. ¹H NMR (300 MHz, CDCl₃): δ 5.34-5.43 (m, 1H), 4.30-4.75 (m, 3H), 3.20-3.32 (m, 1H), 3.00-3.20 (m, 4H), 2.47-2.71 (m, 2H), 2.22-2.43 (m, 2H), 0.93-2.13 (br. m, 58H), 1.02 (s, 3H), 0.91 (d, 3H, J=6.4 Hz), 0.86 (d, 3H, J=6.6 Hz), 0.86 (d, 3H, J=6.6 Hz), 0.68 (s, 3H).

### Step 6: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-JH-cyclopenta[a]phenanthren-3-yl (6-aminohexyl)-L-lysinate trihydrochloride

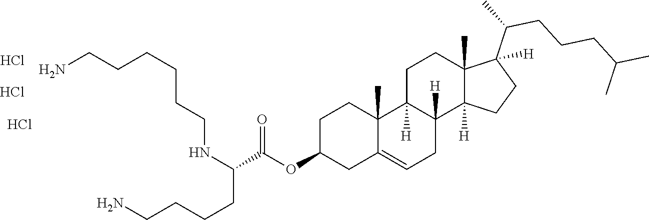

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N₆-(tert-butoxycarbonyl)-N₂-(6-((tert-butoxycarbonyl)amino)hexyl)-L-lysinate (0.379 g, 0.465 mmol) in iPrOH (5.5 mL) was added 5-6 N HCl in iPrOH (0.93 mL). The reaction mixture stirred at 40° C. and was monitored by LCMS. At 16 h, the reaction mixture was cooled to rt, and then iPrOH (30 mL) was added. The suspension was centrifuged (10,000×g for 30 min). The supernatant was decanted, and then solids were suspended in MTBE (35 mL). The suspension was centrifuged (10,000×g for 30 min). The supernatant was decanted, and the solids were suspended in heptanes and then concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)-L-lysinate trihydrochloride (0.133 g, 0.166 mmol, 35.7%) as a white solid. UPLC/ELSD: RT=1.85 min. MS (ES): m/z=328.58 [(M+2H)+CH₃CN]²⁺ for C₃₉H₇₁N₃O₂. ¹H NMR (300 MHz, DMSO): δ 9.85 (br. s, 1H), 9.33 (br. s, 1H), 7.71-8.43 (m, 6H), 5.26-5.52 (m, 1H), 4.50-4.76 (m, 1H), 3.87-4.07 (m, 1H), 2.67-3.06 (m, 6H), 2.25-2.44 (m, 2H), 0.92-2.11 (br. m, 40H), 0.99 (s, 3H), 0.89 (d, 3H, J=6.3 Hz), 0.84 (d, 6H, J=6.5 Hz), 0.65 (s, 3H).

### BW. Compound SA132: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-amino-2-((6-aminohexyl)amino)pentanoate trihydrochloride

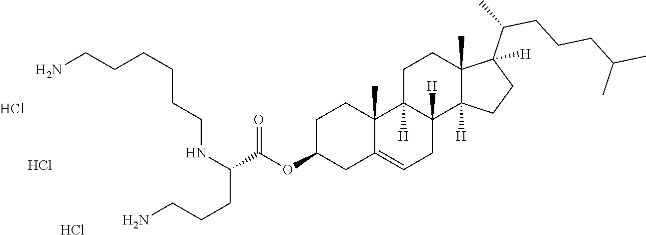

### Step 1: Methyl (2S)-5-[(tert-butoxycarbonyl)amino]-2-(2-nitrobenzenesulfonamido)pentanoate

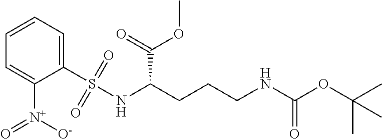 [SMILES: COC(=O)[C@H](CCCNC(=O)OC(C)(C)C)NS(=O)(=O)c1ccccc1[N+](=O)[O-]]

A solution of methyl (S)-2-amino-5-((tert-butoxycarbonyl)amino)pentanoate hydrochloride (1.000 g, 3.537 mmol) and triethylamine (1.50 mL, 10.7 mmol) in DCM (15 mL) was cooled to 0° C. in an ice bath, and then 2-nitrobenzenesulfonyl chloride (0.940 g, 4.24 mmol) in DCM (5.0 mL) was added dropwise. The reaction mixture stirred at rt and was monitored by LCMS. At 17 h, the reaction mixture was cooled to 0° C. in an ice bath, and then water (20 mL) was added. The layers were separated, and the organics were washed with 5% aq. NaHCO₃ solution, passed through a hydrophobic frit, dried over Na₂SO₄, and concentrated. The crude material was purified via silica gel chromatography (30-70% EtOAc in hexanes) to afford methyl (2S)-5-[(tert-butoxycarbonyl)amino]-2-(2-nitrobenzenesulfonamido)pentanoate (1.366 g, 3.166 mmol, 89.5%) as a viscous, yellow oil. UPLC/ELSD: RT=0.68 min. MS (ES): m/z=376.23 [(M+H)—(CH₃)₂C═CH₂]⁺ for C₁₇H₂₅N₃O₈S. ¹H NMR (300 MHz, CDCl₃): δ 8.03-8.10 (m, 1H), 7.88-7.96 (m, 1H), 7.68-7.78 (m, 2H), 6.14 (d, 1H, J=9.0 Hz), 4.55 (br. s, 1H), 4.18 (td, 1H, J=8.4, 5.2 Hz), 3.47 (s, 3H), 3.14 (dt, 2H, J=6.0, 5.7 Hz), 1.82-1.97 (m, 1H), 1.55-1.80 (m, 3H), 1.44 (s, 9H).

### Step 2: Methyl (2S)-5-[(tert-butoxycarbonyl)amino]-2-(N-{6-[(tert-butoxycarbonyl)amino]hexyl}-2-nitrobenzenesulfonamido)pentanoate

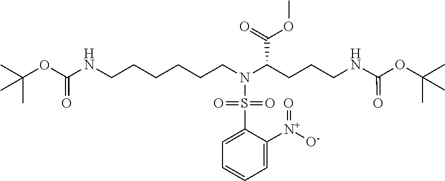 [SMILES: COC(=O)[C@H](CCCNC(=O)OC(C)(C)C)N(CCCCCCNC(=O)OC(C)(C)C)S(=O)(=O)c1ccccc1[N+](=O)[O-]]

Methyl (2S)-5-[(tert-butoxycarbonyl)amino]-2-(2-nitrobenzenesulfonamido)pentanoate (0.600 g, 1.39 mmol), tert-butyl N-(6-bromohexyl)carbamate (0.506 g, 1.81 mmol), potassium carbonate (0.480 g, 3.48 mmol), and potassium iodide (0.046 g, 0.28 mmol) were combined in DMF (9.0 mL). The reaction mixture stirred at 80° C. and was monitored by LCMS. At 2.5 h, the reaction mixture was cooled to rt and then filtered rinsing with MTBE. The filtrate was diluted with MTBE to 80 mL, washed with water (3×) and brine, dried over Na₂SO₄, and concentrated. The crude material was purified via silica gel chromatography (30-70% EtOAc in hexanes) to afford methyl (2S)-5-[(tert-butoxycarbonyl)amino]-2-(N-{6-[(tert-butoxycarbonyl)amino]hexyl}-2-nitrobenzenesulfonamido)pentanoate (0.720 g, 1.141 mmol, 82.1%) as a clear oil. UPLC/ELSD: RT=1.41 min. MS (ES): m/z=475.47 [(M+H)—2((CH₃)₂C═CH₂) —CO₂]⁺ for C₂₈H₄₆N₄O₁₀S. ¹H NMR (300 MHz, CDCl₃): δ 7.99-8.09 (m, 1H), 7.64-7.73 (m, 2H), 7.53-7.61 (m, 1H), 4.42-4.76 (m, 3H), 3.54 (s, 3H), 3.32-3.45 (m, 1H), 2.99-3.23 (m, 5H), 1.99-2.16 (m, 1H), 1.20-1.91 (br. m, 11H), 1.44 (s, 18H).

### Step 3: (2S)-5-[(tert-butoxycarbonyl)amino]-2-(N-[6-[(tert-butoxycarbonyl)amino]hexyl]-2-nitrobenzenesulfonamido)pentanoic acid

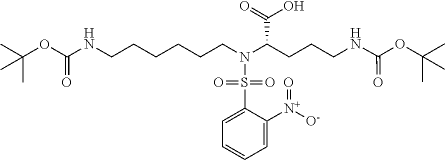 [SMILES: CC(C)(C)OC(=O)NCCCCCCN([C@@H](CCCNC(=O)OC(C)(C)C)C(=O)O)S(=O)(=O)c1ccccc1[N+](=O)[O-]]

To a solution of methyl (2S)-5-[(tert-butoxycarbonyl)amino]-2-(N-{6-[(tert-butoxycarbonyl)amino]hexyl}-2-nitrobenzenesulfonamido)pentanoate (0.716 g, 1.14 mmol) in THF (7.7 mL) and MeOH (1.5 mL) was added aq. lithium hydroxide monohydrate (0.96 mL, 15 w/v %). The reaction mixture stirred at rt and was monitored by LCMS. At 19 h, the reaction mixture was concentrated to remove volatile organics, taken up in water (50 mL), and extracted with EtOAc (3×25 mL). The combined organics were washed with a 5% aq. K₂CO₃ solution and then a 5% aq. citric acid solution, dried over Na₂SO₄, and concentrated to afford (2S)-5-[(tert-butoxycarbonyl)amino]-2-(N-{6-[(tert-butoxycarbonyl)amino]hexyl}-2-nitrobenzenesulfonamido)pentanoic acid (0.619 g, 1.00 mmol, 88.4%) as an amber oil. UPLC/ELSD: RT=1.22 min. MS (ES): m/z=461.4 [(M+H)—2((CH₃)₂C═CH₂)—CO₂]⁺ for C₂₇H₄₄N₄O₁₀S. ¹H NMR (300 MHz, CDCl₃): δ 8.02-8.11 (m, 1H), 7.62-7.72 (m, 2H), 7.54-7.62 (m, 1H), 4.47-4.84 (m, 3H), 2.98-3.44 (m, 6H), 1.94-2.13 (m, 1H), 1.20-1.83 (br. m, 11H), 1.44 (s, 18H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((N-(6-((tert-butoxycarbonyl)amino)hexyl)-2-nitrophenyl)sulfonamido)pentanoate

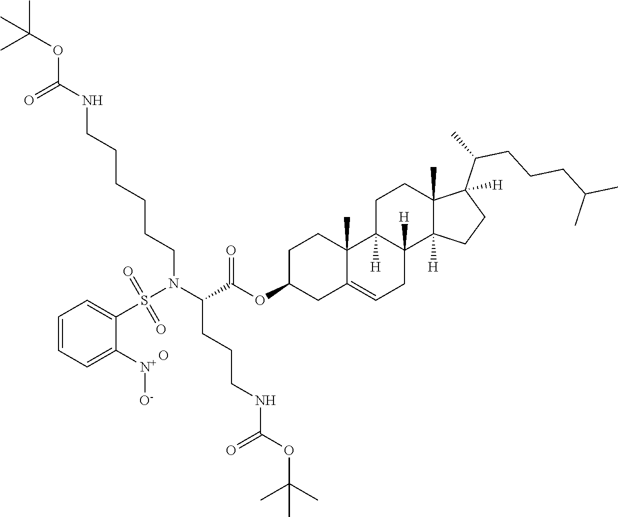

A mixture of (2S)-5-[(tert-butoxycarbonyl)amino]-2-(N-{6-[(tert-butoxycarbonyl)amino]hexyl}-2-nitrobenzenesulfonamido)pentanoic acid (0.520 g, 0.843 mmol), cholesterol (0.359 g, 0.927 mmol), and DMAP (cat.) in DCM (8.0 mL) was cooled to 0° C. Then 1-ethyl-3-(3-dimethylaminopropyl)carbodiimide hydrochloride (0.226 g, 1.18 mmol) was added. The reaction mixture slowly came to rt while stirring and was monitored by LCMS. At 22 h, the reaction mixture was cooled to 0° C., and then DMAP (cat.) and 1-ethyl-3-(3-dimethylaminopropyl)carbodiimide hydrochloride (105 mg) were added. The reaction mixture stirred at rt. At 26 h, the reaction mixture was cooled to 0° C. in an ice bath, and then water (8 mL) was added. The biphasic mixture came to rt and then was separated. The organics were washed with 5% aq. NaHCO₃ solution, passed through a hydrophobic frit, dried over Na₂SO₄, and concentrated. The crude material was purified via silica gel chromatography (0-40% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((N-(6-((tert-butoxycarbonyl)amino)hexyl)-2-nitrophenyl)sulfonamido)pentanoate (0.299 g, 0.303 mmol, 36.0%) as an amber foam. UPLC/ELSD: RT=3.59 min. MS (ES): m/z=829.74 [(M+H)—2((CH₃)₂C═CH₂)—CO₂]⁺ for C₅₄H₈₈N₄O₁₀S. ¹H NMR (300 MHz, CDCl₃): δ 8.00-8.09 (m, 1H), 7.63-7.74 (m, 2H), 7.53-7.60 (m, 1H), 5.23-5.35 (m, 1H), 4.35-4.84 (m, 4H), 3.33-3.53 (m, 1H), 2.93-3.27 (m, 5H), 0.94-2.17 (br. m, 58H), 0.92 (s, 3H), 0.90 (d, 3H, J=6.5 Hz), 0.86 (d, 3H, J=6.6 Hz), 0.86 (d, 3H, J=6.6 Hz), 0.66 (s, 3H).

### Step 5: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((6-((tert-butoxycarbonyl)amino)hexyl)amino)pentanoate

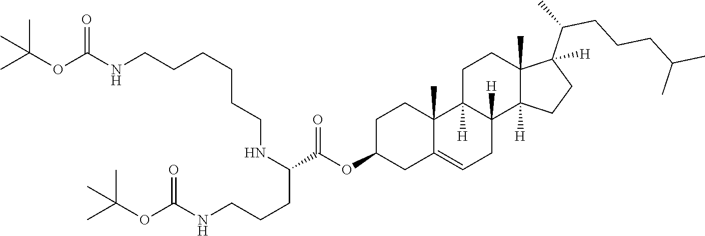

To a mixture of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((N-(6-((tert-butoxycarbonyl)amino)hexyl)-2-nitrophenyl)sulfonamido)pentanoate (0.284 g, 0.288 mmol) and potassium carbonate (0.119 g, 0.865 mmol) in DMF (5.0 mL) was added thiophenol (0.05 mL, 0.49 mmol). The reaction mixture stirred at rt and was monitored by LCMS. At 3 h, DCM (10 mL) was added, and the reaction mixture was filtered through a pad of Celite®. The filtrate was diluted with DCM to 80 mL and then was washed once with a 5% aq. NaHCO₃ solution and three times with water. The organics were passed through a hydrophobic frit, dried over Na₂SO₄, and concentrated. The crude material was purified via silica gel chromatography (40-80% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((6-((tert-butoxycarbonyl)amino)hexyl)amino)pentanoate (0.203 g, 0.254 mmol, 88.0%) as a clear, viscous oil. UPLC/ELSD: RT=2.92 min. MS (ES): m/z=801.37 [M+H]⁺ for C₄₈H₈₅N₃O₆. ¹H NMR (300 MHz, CDCl₃): δ 5.34-5.42 (m, 1H), 5.03-5.13 (m, 1H), 4.45-4.81 (m, 2H), 3.50-3.81 (m, 1H), 2.82-3.32 (m, 6H), 2.22-2.48 (m, 2H), 0.94-2.19 (br. m, 56H), 1.02 (s, 3H), 0.91 (d, 3H, J=6.4 Hz), 0.87 (d, 3H, J=6.6 Hz), 0.86 (d, 3H, J=6.6 Hz), 0.68 (s, 3H).

### Step 6: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-JH-cyclopenta[a]phenanthren-3-yl (S)-5-amino-2-((6-aminohexyl)amino)pentanoate trihydrochloride

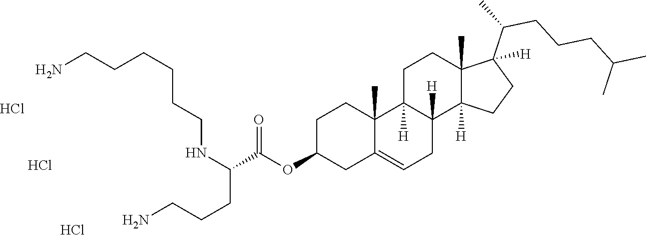

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((6-((tert-butoxycarbonyl)amino)hexyl)amino)pentanoate (0.197 g, 0.246 mmol) in iPrOH (3.0 mL) was added 5-6 N HCl in iPrOH (0.49 mL). The reaction mixture stirred at 40° C. and was monitored by LCMS. At 16 h, the reaction mixture was cooled to rt, and then ACN (9 mL) was added. The suspension was filtered, but particles passed through frit. The suspension was concentrated, and the residue suspended in MTBE (30 mL). The suspension was centrifuged (10,000×g for 30 min), and then the supernatant was decanted. Solids were suspended in heptanes, and then concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-amino-2-((6-aminohexyl)amino)pentanoate trihydrochloride (0.147 g, 0.170 mmol, 69.0%) as a white solid. UPLC/ELSD: RT=1.86 min. MS (ES): m/z=321.42 [(M+2H)+CH₃CN]²⁺ for C₃₈H₆₉N₃O₂. ¹H NMR (300 MHz, DMSO): δ 9.75 (br. s, 1H), 9.40 (br. s, 1H), 7.61-8.32 (m, 6H), 5.31-5.48 (m, 1H), 4.53-4.72 (m, 1H), 3.92-4.16 (m, 1H), 2.66-3.05 (m, 6H), 2.24-2.45 (m, 2H), 0.92-2.13 (br. m, 38H), 1.00 (s, 3H), 0.90 (d, 3H, J=6.3 Hz), 0.84 (d, 3H, J=6.6 Hz), 0.84 (d, 3H, J=6.6 Hz), 0.66 (s, 3H).

### BX. Compound SA133: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride

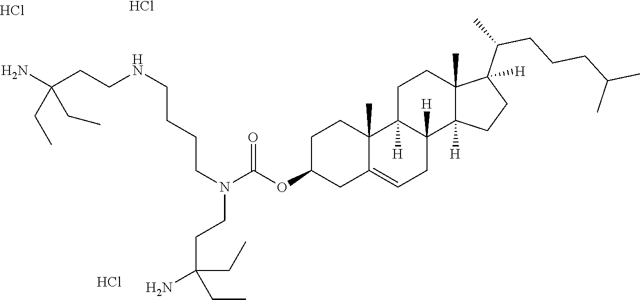

### Step 1: tert-butyl (3-ethyl-1-((2-nitrophenyl)sulfonamido)pentan-3-yl)carbamate

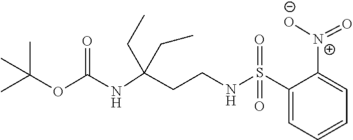 [SMILES: CCC(CC)(CCNS(=O)(=O)c1ccccc1[N+](=O)[O-])NC(=O)OC(C)(C)C]

To a solution of tert-butyl N-(1-amino-3-ethylpentan-3-yl)carbamate (2.50 g, 10.31 mmol) in dry DCM (50 mL) set stirring under nitrogen was added triethylamine (2.87 mL, 20.62 mmol). The solution was cooled to 0° C. and then a solution of 2-nitrobenzenesulfonyl chloride (2.51 g, 11.34 mmol) in 50 mL dry DCM was added dropwise over 30 minutes. The reaction was allowed to proceed at 0° C. for an hour and then at room temperature for an additional three hours. The mixture was then diluted with an additional 10 mL DCM, washed with saturated aqueous sodium bicarbonate (1×100 mL), water (1×100 mL), 10% aqueous citric acid (1×100 mL), water (1×100 mL), and brine (1×100 mL), dried over sodium sulfate, filtered, and concentrated to give tert-butyl (3-ethyl-1-((2-nitrophenyl)sulfonamido)pentan-3-yl)carbamate as a white solid (4.48 g, 10.31 mmol, quantitative). UPLC/ELSD: RT=1.27 min. MS (ES): m/z (MH⁺) 415.5 for C₁₈H₂₉N₃O₆S. ¹H NMR (300 MHz, CDCl₃) δ: ppm 8.12 (m, 1H), 7.85 (m, 1H), 7.76 (m, 2H), 5.41 (br. s, 1H), 4.19 (br. s, 1H), 3.14 (m, 2H), 1.92 (t, 2H), 1.63 (m, 2H), 1.45 (m, 2H), 1.40 (s, 9H), 0.78 (t, 6H).

### Step 2: di-tert-butyl ((butane-1,4-diylbis(azanediyl))bis(3-ethylpentane-1, 3-diyl))dicarbamate

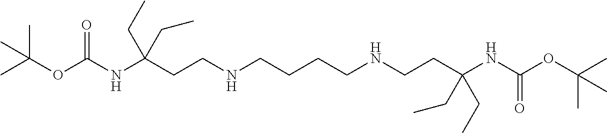 [SMILES: CCC(CC)(CCNCCCCNCCC(CC)(CC)NC(=O)OC(C)(C)C)NC(=O)OC(C)(C)C]

To a solution of tert-butyl (3-ethyl-1-((2-nitrophenyl)sulfonamido)pentan-3-yl)carbamate (4.48 g, 10.78 mmol) in dry DMF (50 mL) set stirring under nitrogen was added potassium carbonate (4.33 g, 31.32 mmol) and 1,4-diiodobutane (0.68 mL, 5.13 mmol). The solution was heated to 40° C. and allowed to proceed overnight. The following morning, benzyl bromide (0.51 mL, 4.26 mmol) was added and the reaction was allowed to proceed at room temperature for 8 h. Then, thiophenol (2.02 mL, 19.77 mmol), potassium carbonate (2.13 g, 15.40 mmol), and an additional 20 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×10 mL), dried over potassium carbonate, filtered, and concentrated to an oil. The oil was taken up again in DCM and purified via silica gel chromatography in DCM with a 0-50% (50:45:5 DCM/MeOH/concentrated aqueous ammonium hydroxide) gradient. Fractions containing product were combined and concentrated to give di-tert-butyl ((butane-1,4-diylbis(azanediyl))bis(3-ethylpentane-1,3-diyl))dicarbamate as a colorless oil (2.14 g, 4.17 mmol, 81.1%). UPLC/ELSD: RT=2.52 min. MS (ES): m/z (MH⁺) 515.6 for C₂₈H₅₈N₄O₄. ¹H NMR (300 MHz, CDCl₃) δ: ppm 5.20 (m, 2H), 2.45 (br. m, 8H), 1.48 (m, 13H), 1.35 (s, 4H), 1.23 (s, 19H), 0.62 (t, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-((tert-butoxycarbonyl)amino)-3-ethylpentyl)amino)butyl)carbamate

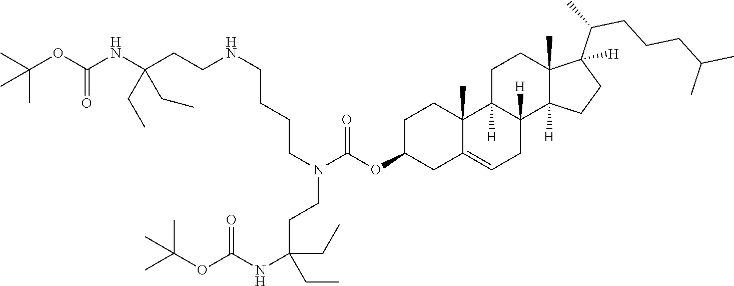

To a solution of di-tert-butyl ((butane-1,4-diylbis(azanediyl))bis(3-ethylpentane-1,3-diyl))dicarbamate (0.50 g, 0.97 mmol) in dry toluene (10 mL) set stirring under nitrogen was added triethylamine (0.41 mL, 2.91 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.54 g, 0.97 mmol) was added and the solution was heated to 90° C. and allowed to proceed overnight. The following morning, the reaction mixture was allowed to cool to room temperature, diluted with toluene, washed with water (3×15 mL), dried over sodium sulfate, filtered, and concentrated to an oil. The oil was taken up in DCM and purified via silica gel chromatography in DCM with a 0-30% (50:45:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-((tert-butoxycarbonyl)amino)-3-ethylpentyl)amino)butyl)carbamate as a colorless oil (0.55 g, 0.59 mmol, 60.9%). UPLC/ELSD: RT=3.06 min. MS (ES): m/z (MH⁺) 928.3 for C₅₆H₁₀₂N₄O₆. ¹H NMR (300 MHz, CDCl₃) δ: ppm 5.34 (br. m, 1H), 5.02 (m, 1H), 4.46 (br. m, 3H), 3.18 (br. m, 4H), 2.56 (m, 4H), 2.28 (m, 2H), 1.83 (m, 6H), 1.58 (br. m, 16H), 1.39 (s, 18H), 1.10 (br. m, 11H), 0.97 (s, 5H), 0.88 (d, 3H, J=6 Hz), 0.79 (m, 18H), 0.64 (s, 4H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride

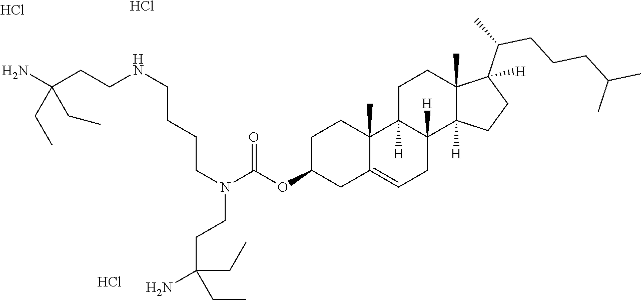

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-((tert-butoxycarbonyl)amino)-3-ethylpentyl)amino)butyl)carbamate (0.55 g, 0.59 mmol) in isopropanol (10 mL) set stirring under nitrogen was added hydrochloric acid (5 N in isopropanol, 1.18 mL, 5.90 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride as a white solid (0.34 g, 0.40 mmol, 68.1%). UPLC/ELSD: RT=2.08 min. MS (ES): m/z (MH⁺) 728.2 for C₄₆H₈₉Cl₃N₄O₂. ¹H NMR (300 MHz, MeOD) δ: ppm 5.45 (m, 1H), 4.48 (br. m, 1H), 3.94 (m, 1H), 3.37 (br. m, 3H), 3.14 (m, 4H), 2.40 (m, 2H), 2.11 (m, 3H), 1.93 (br. m, 6H), 1.74 (br. m, 13H), 1.55 (m, 12H), 1.18 (d, 14H, J=6 Hz), 1.04 (br. m, 17H), 0.98 (d, 4H, J=6 Hz), 0.91 (d, 6H, J=6 Hz), 0.74 (s, 3H).

### BY. Compound SA134: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride

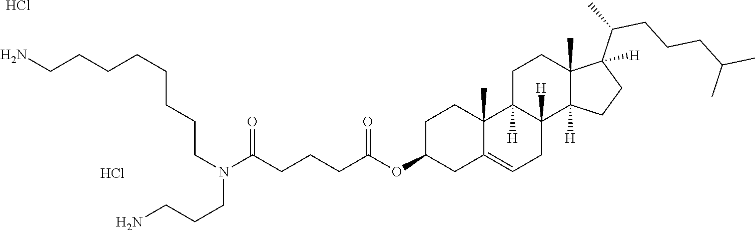

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate

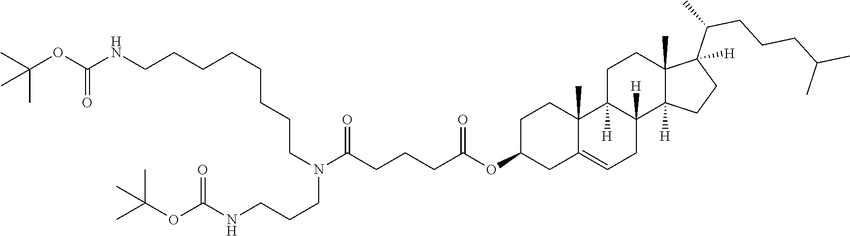

To a solution of 5-{[(1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl]oxy}-5-oxopentanoic acid (0.24 g, 0.48 mmol) in dry DCM (10 mL) stirring under nitrogen was added tert-butyl N-[3-({8-[(tert-butoxycarbonyl)amino]octyl}amino)propyl]carbamate (0.19 g, 0.48 mmol), dimethylaminopyridine (0.12 g, 0.95 mmol), and 1-ethyl-3-(3-dimethylaminopropyl)carbodiimide hydrochloride (0.18 g, 0.95 mmol). The resulting solution was stirred at room temperature and proceeded overnight. Then the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate as a light yellow oil (0.32 g, 0.37 mmol, 77.0%). UPLC/ELSD: RT: 3.52 min. MS (ES): m/z (MH⁺) 885.4 for C₅₃H₉₃N₃O₇. ¹H NMR (300 MHz, CDCl₃) δ: ppm 5.17 (m, 1H), 5.02 (m, 1H), 4.43 (br. m, 1H), 4.29 (br. m, 1H), 3.06 (m, 2H), 2.95 (m, 1H), 2.86 (m, 2H) 2.73 (m, 4H), 2.04 (br. m, 6H), 1.62 (br. m, 4H), 1.51 (m, 4H), 1.20 (br. m, 12H), 1.10 (s, 19H), 0.97 (br. m, 13H), 0.81 (br. m, 7H), 0.68 (s, 6H), 0.60 (d, 4H, J=6 Hz), 0.54 (d, 6H, J=6 Hz), 0.35 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride

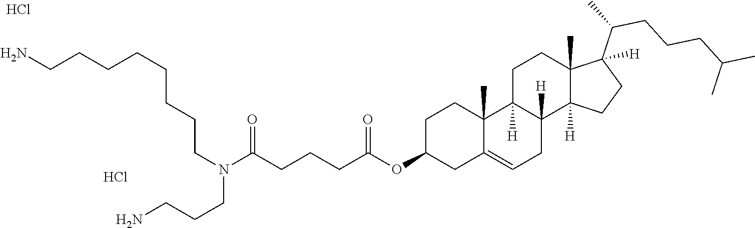

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate (0.32 g, 0.37 mmol) in isopropanol (5 mL) set stirring under nitrogen was added hydrochloric acid (5 N in isopropanol, 0.73 mL, 3.66 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride as a white solid (0.16 g, 0.19 mmol, 53.1%). UPLC/ELSD: RT=1.99 min. MS (ES): m/z (MH⁺) 684.9 for C₄₃H₇₉Cl₂N₃O₃. ¹H NMR (300 MHz, MeOD) δ: ppm 5.42 (m, 1H), 4.55 (br. m, 1H), 3.48 (t, 2H), 3.36 (m, 2H), 2.94 (m, 4H), 2.49 (t, 2H), 2.40 (m, 4H), 1.92 (br. m, 9H), 1.63 (br. m, 11H), 1.41 (br. m, 12H), 1.16 (m, 8H), 1.07 (s, 5H), 0.97 (d, 4H, J=6 Hz), 0.90 (d, 6H, J=6 Hz), 0.75 (s, 3H).

### BZ. Compound SA135: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride

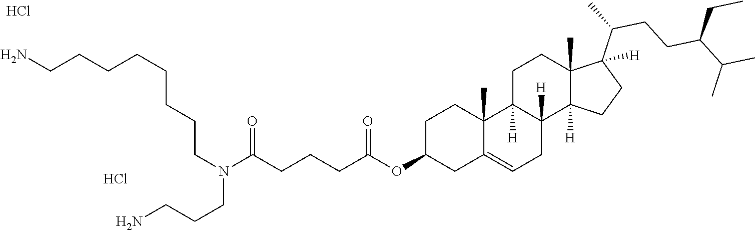

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate

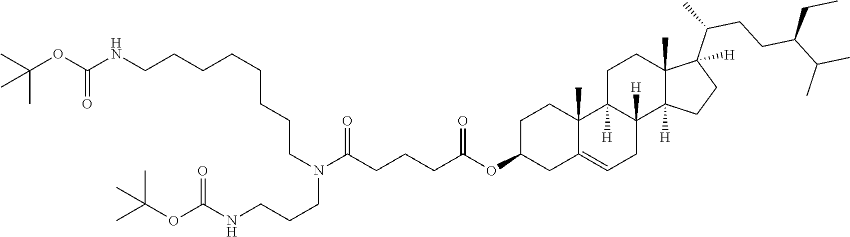

To a solution of 5-{[(1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,10H,11H-cyclopenta[a]phenanthren-7-yl]oxy}-5-oxopentanoic acid (0.24 g, 0.45 mmol) in dry DCM (10 mL) stirring under nitrogen was added tert-butyl N-[3-({8-[(tert-butoxycarbonyl)amino]octyl}amino)propyl]carbamate (0.18 g, 0.45 mmol), dimethylaminopyridine (0.11 g, 0.90 mmol), and 1-ethyl-3-(3-dimethylaminopropyl)carbodiimide hydrochloride (0.17 g, 0.90 mmol). The resulting solution was stirred at room temperature and proceeded overnight. Then the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate as a light yellow oil (0.27 g, 0.30 mmol, 67.0%). UPLC/ELSD: RT: 3.60 min. MS (ES): m/z (MH⁺) 913.4 for C₅₅H₉₇N₃O₇. ¹H NMR (300 MHz, CDCl₃) δ: ppm 5.03 (m, 1H), 4.44 (m, 1H), 4.25 (br. m, 1H), 3.06 (br. m, 2H), 2.95 (m, 1H), 2.86 (m, 2H) 2.74 (m, 4H), 2.04 (br. m, 6H), 1.62 (br. m, 4H), 1.50 (m, 4H), 1.32 (br. m, 11H), 1.10 (s, 19H), 0.97 (br. m, 12H), 0.79 (br. m, 7H), 0.68 (s, 5H), 0.60 (d, 5H, J=6 Hz), 0.51 (q, 9H), 0.35 (s, 4H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride

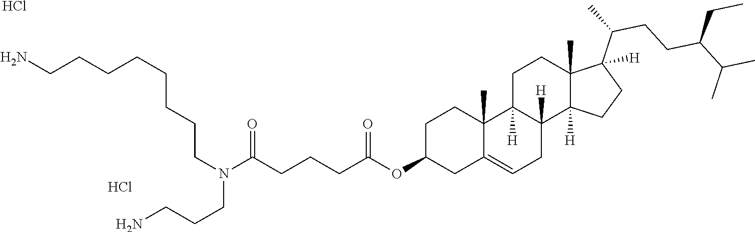

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate (0.27 g, 0.30 mmol) in isopropanol (5 mL) set stirring under nitrogen was added hydrochloric acid (5 N in isopropanol, 0.59 mL, 2.96 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. The white solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride as a white solid (0.12 g, 0.12 mmol, 44.6%). UPLC/ELSD: RT=2.23 min. MS (ES): m/z (MH⁺) 712.8 for C₄₅H₈₃C₂N₃O₃. ¹H NMR (300 MHz, MeOD) δ: ppm 5.40 (m, 1H), 4.55 (br. m, 1H), 3.48 (t, 2H), 3.33 (m, 1H), 2.91 (m, 3H), 2.49 (t, 2H), 2.40 (m, 4H), 1.91 (br. m, 7H), 1.66 (br. m, 11H), 1.41 (br. m, 15H), 1.18 (d, 6H, J=6 Hz), 1.07 (s, 6H), 0.99 (d, 5H, J=6 Hz), 0.89 (q, 9H), 0.75 (s, 3H).

### CA. Compound SA136: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride

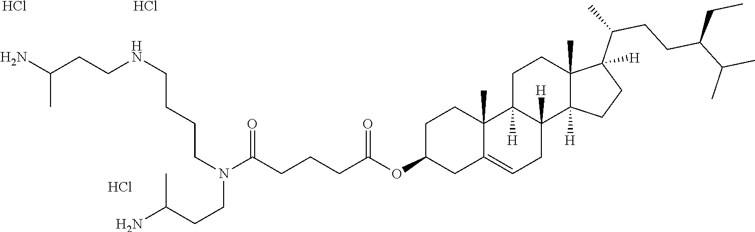

### Step 1: (3S,8S,9S,10R,13R,14S,17R)-17-((2R,5R)-5-ethyl-6-methylheptan-2-yl)-10,13-dimethyl-2,3,4, 7, 8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate

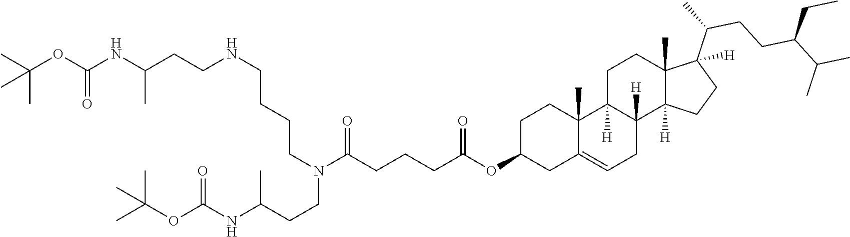

To a solution of 5-{[(1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,101H,11H-cyclopenta[a]phenanthren-7-yl]oxy}-5-oxopentanoic acid (0.33 g, 0.61 mmol) in dry DCM (10 mL) stirring under nitrogen was added tert-butyl N-(4-{[4-({3-[(tert-butoxycarbonyl)amino]butyl}amino)butyl]amino}butan-2-yl)carbamate (0.79 g, 1.83 mmol), dimethylaminopyridine (0.15 g, 1.22 mmol), and 1-ethyl-3-(3-dimethylaminopropyl)carbodiimide hydrochloride (0.24 g, 1.22 mmol). The resulting solution was stirred at room temperature and proceeded overnight. Then the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate as a light yellow oil (0.12 g, 0.12 mmol, 20.2%). UPLC/ELSD: RT: 2.81 min. MS (ES): m/z (MH⁺) 942.4 for C₅₆H₁₀₀N₄O₇. ¹H NMR (300 MHz, CDCl₃) δ: ppm 5.33 (m, 1H), 4.85 (m, 3H), 3.70 (br. m, 1H), 3.23 (br. m, 5H), 2.56 (br. m, 4H), 2.32 (m, 7H) 1.91 (m, 8H), 1.56 (br. m, 12H), 1.40 (s, 21H), 1.21 (m, 6H), 1.12 (m, 11H), 0.98 (s, 5H), 0.90 (d, 5H, J=6 Hz), 0.79 (q, 9H), 0.65 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride

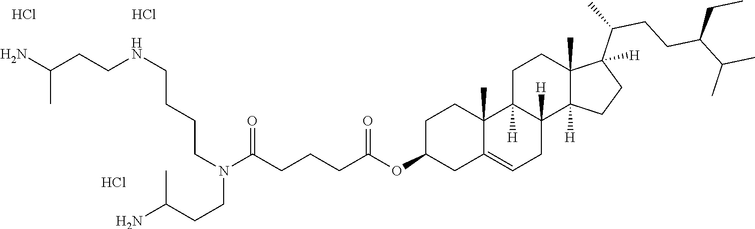

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate (0.12 g, 0.12 mmol) in isopropanol (5 mL) set stirring under nitrogen was added hydrochloric acid (5 N in isopropanol, 0.25 mL, 1.23 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride as a white solid (0.05 g, 0.05 mmol, 42.5%). UPLC/ELSD: RT=1.90 min. MS (ES): m/z (MH⁺) 742.0 for C₄₆H₈₇Cl₃N₄O₃. ¹H NMR (300 MHz, MeOD) δ: ppm 5.42 (m, 1H), 4.57 (br. m, 1H), 3.67 (m, 1H), 3.48 (m, 5H), 3.18 (m, 5H), 2.42 (m, 6H), 1.92 (br. m, 22H), 1.39 (m, 10H), 1.20 (m, 8H), 1.07 (s, 5H), 0.98 (d, 5H, J=6 Hz), 0.87 (q, 9H), 0.75 (s, 3H).

### CB. Compound SA137: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride

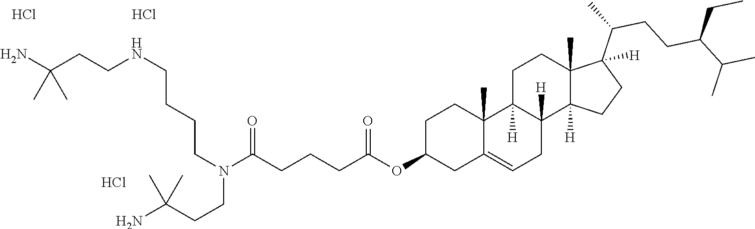

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate

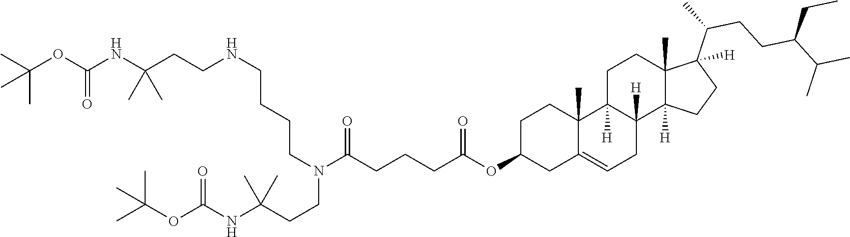

To a solution of 5-{[(1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,10H,11H-cyclopenta[a]phenanthren-7-yl]oxy}-5-oxopentanoic acid (0.31 g, 0.58 mmol) in dry DCM (10 mL) stirring under nitrogen was added tert-butyl N-(4-{[4-({3-[(tert-butoxycarbonyl)amino]-3-methylbutyl}amino)butyl]amino}-2-methylbutan-2-yl)carbamate (0.80 g, 1.75 mmol), dimethylaminopyridine (0.14 g, 1.17 mmol), and 1-ethyl-3-(3-dimethylaminopropyl)carbodiimide hydrochloride (0.23 g, 1.17 mmol). The resulting solution was stirred at room temperature and proceeded overnight. Then the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7, 8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate as a light yellow oil (0.20 g, 0.20 mmol, 35.0%). UPLC/ELSD: RT: 2.86 min. MS (ES): m/z (MH⁺) 970.4 for C₅₈H₁₀₄N₄O₇. ¹H NMR (300 MHz, CDCl₃) δ: ppm 5.54 (m, 1H), 5.04 (m, 1H), 4.23 (m, 2H), 2.91 (br. m, 4H), 2.35 (br. m, 4H), 2.03 (br. m, 6H), 1.61 (m, 8H) 1.35 (m, 10H), 1.10 (s, 19H), 0.94 (m, 15H), 0.83 (m, 6H), 0.68 (s, 6H), 0.60 (m, 5H), 0.49 (q, 9H), 0.35 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-H-cyclopenta[a]phenanthren-3-yl 5-((3-amino-3-methylbutyl)(4-((3-amino-3-methylbutyl)amino)butyl)amino)-5-oxopentanoate trihydrochloride

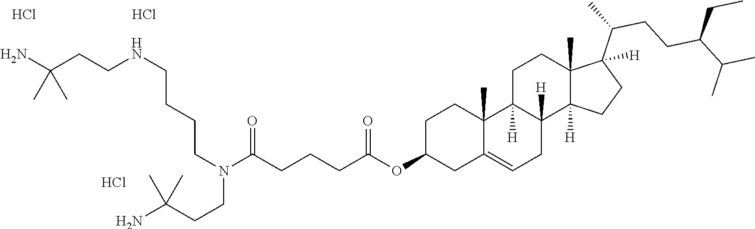

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate (0.20 g, 0.20 mmol) in isopropanol (5 mL) set stirring under nitrogen was added hydrochloric acid (5 N in isopropanol, 0.41 mL, 2.04 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride as a white solid (0.11 g, 0.11 mmol, 54.0%). UPLC/ELSD: RT=1.94 min. MS (ES): m/z (MH⁺) 770.0 for C₄₈H₉₁Cl₃N₄O₃. ¹H NMR (300 MHz, MeOD) δ: ppm 5.42 (m, 1H), 4.55 (br. m, 1H), 3.45 (m, 4H), 3.16 (m, 4H), 2.41 (m, 6H), 1.89 (br. m, 22H), 1.43 (m, 14H), 1.27 (m, 7H), 1.18 (m, 4H), 1.07 (s, 6H), 0.98 (d, 5H, J=6 Hz), 0.89 (q, 9H), 0.75 (s, 3H).

### CC. Compound SA138: N-(3-amino-3-methylbutyl)-N-(4-((3-amino-3-methylbutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

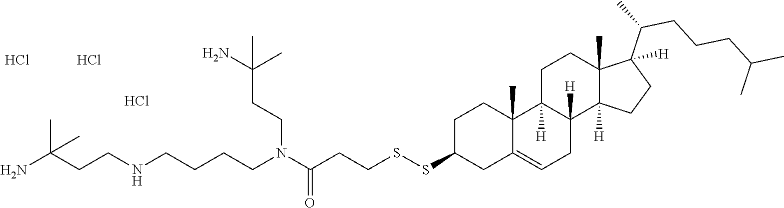

### Step 1: tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6,6,17-pentamethyl-4-oxo-3-oxa-5,9,14-triazaoctadecan-17-yl)carbamate

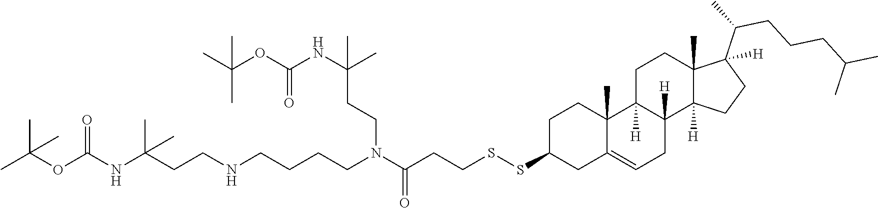

A solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.250 g, 0.493 mmol), tert-butyl N-(4-{[4-({3-[(tert-butoxycarbonyl)amino]-3-methylbutyl}amino)butyl]amino}-2-methylbutan-2-yl)carbamate (0.452 g, 0.986 mmol), and triethylamine (0.20 mL, 1.4 mmol) in DCM (2.5 mL) was cooled to 0° C. in an ice bath, and then propanephosphonic acid anhydride (50 wt % in DCM) (0.62 g, 0.97 mmol) was added dropwise. The reaction mixture stirred at rt and was monitored by LCMS. At 1.5 h, the reaction mixture was cooled to 0° C. in an ice bath, and 5% aq. NaHCO₃ solution (10 mL) was added. The reaction mixture then stirred at rt for 10 min. After this time, the mixture was extracted with DCM (3×15 mL). The combined organics were passed through a hydrophobic frit, dried over Na₂SO₄, and concentrated. The crude material was purified via silica gel chromatography (0-12% (5% conc. aq. NH₄OH in MeOH) in DCM) to afford tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6,6,17-pentamethyl-4-oxo-3-oxa-5,9,14-triazaoctadecan-17-yl)carbamate (0.269 g, 0.284 mmol, 57.6%) as a clear gel. UPLC/ELSD: RT=2.90 min. MS (ES): m/z=948.55 [M+H]⁺ for C₅₄H₉₈N₄O₅S₂. ¹H NMR (300 MHz, CDCl₃): δ 5.32-5.39 (m, 1H), 3.18-3.58 (m, 6H), 2.43-3.03 (m, 9H), 2.26-2.40 (m, 2H), 0.91-2.18 (br. m, 64H), 1.00 (s, 3H), 0.91 (d, 3H, J=6.5 Hz), 0.86 (d, 3H, J=6.6 Hz), 0.86 (d, 3H, J=6.6 Hz), 0.67 (s, 3H).

### Step 2: N-(3-amino-3-methylbutyl)-N-(4-((3-amino-3-methylbutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

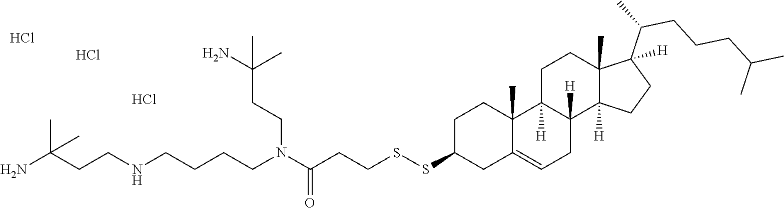

To a solution of tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6,6,17-pentamethyl-4-oxo-3-oxa-5,9,14-triazaoctadecan-17-yl)carbamate (0.266 g, 0.281 mmol) in DCM (2.6 mL) in a screwcap vial was added 4 N HCl in dioxane (0.49 mL). The reaction mixture stirred at rt and was monitored by LCMS. At 2 h, the reaction mixture was diluted with MTBE to 30 mL, and then centrifuged (10,000×g for 30 min). The supernatant was decanted. The solids were suspended in MTBE and then concentrated to afford N-(3-amino-3-methylbutyl)-N-(4-((3-amino-3-methylbutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride (0.203 g, 0.225 mmol, 80.3%) as a white solid. UPLC/ELSD: RT=1.96 min. MS (ES): m/z=264.75 [(M+3H)+CH₃CN]³⁺ for C₄₄H₈₂N₄OS₂. ¹H NMR (300 MHz, CD₃OD): δ 5.35-5.42 (m, 1H), 3.37-3.59 (m, 4H), 3.05-3.25 (m, 4H), 2.92-3.03 (m, 2H), 2.76-2.89 (m, 2H), 2.57-2.74 (m, 1H), 2.25-2.42 (m, 2H), 0.96-2.18 (br. m, 46H), 1.03 (s, 3H), 0.95 (d, 3H, J=6.5 Hz), 0.88 (d, 6H, J=6.6 Hz), 0.72 (s, 3H).

### CD. Compound SA139: N-(3-amino-3-methylbutyl)-N-(8-amino-8-methylnonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

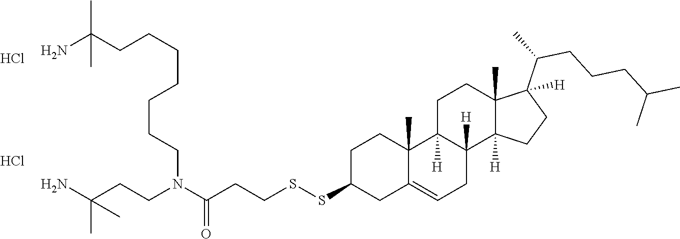

### Step 1: 4-methoxybenzyl (9-(N-(3-((tert-butoxycarbonyl)amino)-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylnonan-2-yl)carbamate

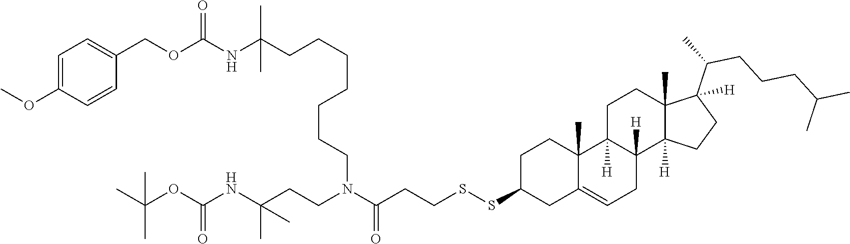

To a stirred solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.100 g, 0.197 mmol), tert-butyl N-(4-{[8-({[(4-methoxyphenyl)methoxy]carbonyl}amino)-8-methylnonyl]amino}-2-methylbutan-2-yl)carbamate (0.103 g, 0.197 mmol), and triethylamine (0.09 mL, 0.6 mmol) in DCM (1.0 mL) cooled to 0° C. was added 50 wt % propanephosphonic acid anhydride in DCM (0.20 mL, 0.39 mmol) dropwise. The reaction mixture was stirred at room temperature and was monitored by LCMS. At 16 hours, the reaction mixture was diluted with DCM (10 mL), and then washed with 5% aq. NaHCO₃ soln. The aqueous layer was extracted with DCM (10 mL). The combined organic layers were passed through a hydrophobic frit, dried over Na₂SO₄, and concentrated. The crude material was purified via silica gel chromatography (0-50% EtOAc in hexanes) to afford 4-methoxybenzyl (9-(N-(3-((tert-butoxycarbonyl)amino)-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylnonan-2-yl)carbamate (0.146 g, 0.144 mmol, 73.2%) as a clear oil. UPLC/ELSD: RT=3.80 min. MS (ES): m/z=1012.83 (M+H)⁺ for C₅₉H₉₉N₃O₆S2. ¹H NMR (300 MHz, CDCl₃) δ 7.32-7.26 (m, 2H), 6.93-6.84 (m, 2H), 5.40-5.31 (m, 1H), 4.97 (s, 2H), 4.73-4.34 (m, 2H), 3.80 (s, 3H), 3.37-3.17 (m, 4H), 3.03-2.88 (m, 2H), 2.76-2.56 (m, 3H), 2.42-2.25 (m, 2H), 2.12-0.94 (m, 61H), 0.99 (s, 3H), 0.91 (d, J=6.4 Hz, 3H), 0.86 (d, J=6.6 Hz, 6H), 0.67 (s, 3H).

### Step 2: N-(3-amino-3-methylbutyl)-N-(8-amino-8-methylnonyl)-3-(((3S,8S,9S,IOR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

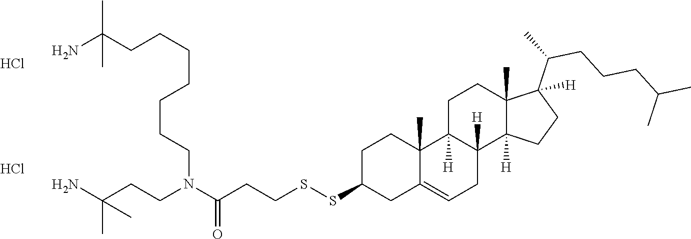

To a stirred solution of 4-methoxybenzyl (9-(N-(3-((tert-butoxycarbonyl)amino)-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylnonan-2-yl)carbamate (0.143 g, 0.142 mmol) in DCM (2.5 mL) cooled to 0° C. was added 4 N HCl in dioxane (0.25 mL). The reaction mixture was allowed to come to room temperature slowly while stirring and was monitored by LCMS. At 22 hours, 4 N HCl in dioxane (0.10 mL) was added. At 27 hours, MTBE (20 mL) added, and the reaction mixture was held at 4° C. overnight. The suspension was centrifuged (10,000×g for 30 min at 4° C.). The supernatant was decanted, the solids were suspended in MTBE, then concentrated to afford N-(3-amino-3-methylbutyl)-N-(8-amino-8-methylnonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride (0.067 g, 0.078 mmol, 55.0%) as a white solid. UPLC/ELSD: RT=2.33 min. MS (ES): m/z=374.56 (M+2H)²′ for C₄₅H₈₃N₃OS₂. ¹H NMR (300 MHz, MeOD) δ 5.45-5.33 (m, 1H), 3.53-3.34 (m, 4H), 3.01-2.89 (m, 2H), 2.85-2.75 (m, 2H), 2.74-2.57 (m, 1H), 2.40-2.27 (m, 2H), 2.14-1.77 (m, 7H), 1.73-0.97 (m, 33H), 1.37 (s, 6H), 1.33 (s, 6H), 1.03 (s, 3H), 0.95 (d, J=6.5 Hz, 3H), 0.89 (d, J=6.6 Hz, 3H), 0.88 (d, J=6.7 Hz, 3H), 0.73 (s, 3H).

### CE. Compound SA141: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride

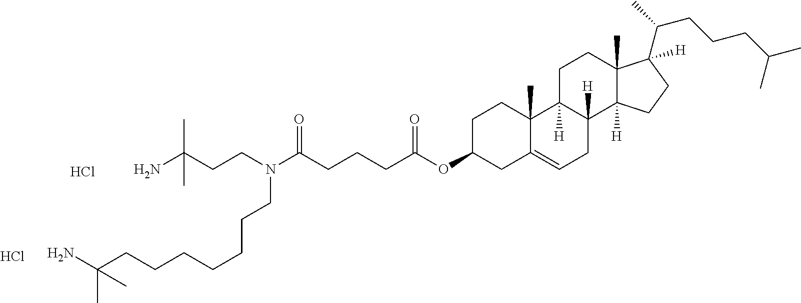

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate

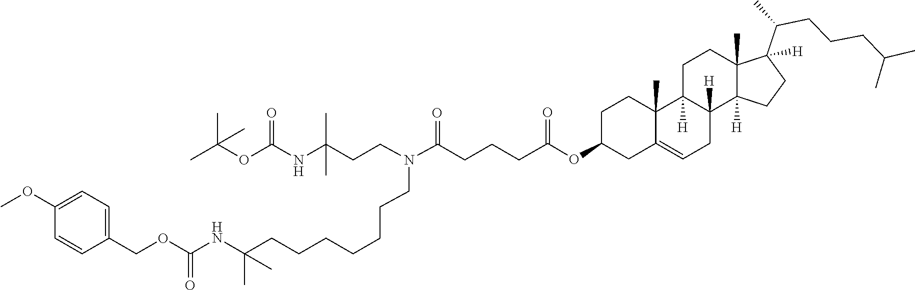

To a stirred solution of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (0.100 g, 0.200 mmol), tert-butyl N-(4-{[8-({[(4-methoxyphenyl)methoxy]carbonyl}amino)-8-methylnonyl]amino}-2-methylbutan-2-yl)carbamate (0.104 g, 0.200 mmol), and DMAP (0.049 g, 0.40 mmol) in DCM (2.0 mL) was added 1-ethyl-3-(3-dimethylaminopropyl)carbodiimide hydrochloride (0.077 g, 0.40 mmol). The reaction mixture was stirred at room temperature and was monitored by LCMS. At 15 hours, the reaction mixture was diluted with DCM (15 mL) and washed with 5% aq. NaHCO₃ soln. The aqueous was extracted with DCM (15 mL). The combined organics were passed through a hydrophobic frit, dried over Na₂SO₄, and concentrated. The crude material was purified via silica gel chromatography (0-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate (0.158 g, 0.157 mmol, 78.8%) as a clear oil. UPLC/ELSD: RT=3.68 min. MS (ES): m/z=1005.92 (M+H)⁺ for C₆₁H₁₀₁N₃O₈. ¹H NMR (300 MHz, CDCl₃) δ 7.32-7.26 (m, 2H), 6.92-6.84 (m, 2H), 5.41-5.30 (m, 1H), 4.97 (s, 2H), 4.76-4.34 (m, 3H), 3.80 (s, 3H), 3.37-3.13 (m, 4H), 2.41-2.24 (m, 6H), 2.08-0.94 (m, 63H), 1.01 (s, 3H), 0.91 (d, J=6.4 Hz, 3H), 0.86 (d, J=6.6 Hz, 3H), 0.86 (d, J=6.6 Hz, 3H), 0.67 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride

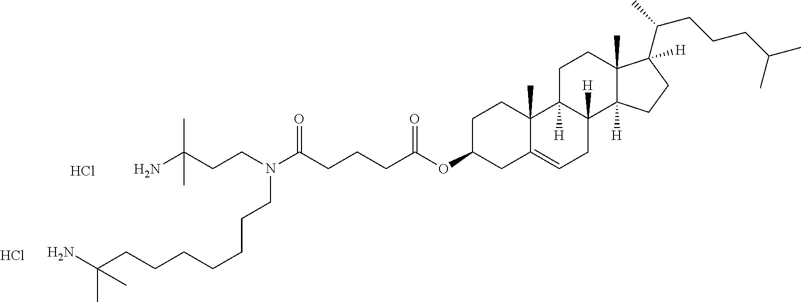

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate (0.143 g, 0.143 mmol) in DCM (2.2 mL) was added 4 N HCl in dioxane (0.25 mL). The reaction mixture was stirred at room temperature and was monitored by LCMS. At 17 hours, 4 N HCl in dioxane (0.10 mL) was added. At 22 hours, MTBE (15 mL) added, and the reaction mixture was held at 4° C. overnight. The suspension was centrifuged (10,000×g for 30 min at 4° C.). The supernatant was decanted. The solids were suspended in MTBE and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride (0.082 g, 0.098 mmol, 68.7%) as a white solid. UPLC/ELSD: RT=2.24 min. MS (ES): m/z=371.23 (M+2H)²⁺ for C₄₇H₈₅N₃O₃. ¹H NMR (300 MHz, MeOD) δ 5.43-5.35 (m, 1H), 4.62-4.47 (m, 1H), 3.51-3.33 (m, 4H), 2.49-2.27 (m, 6H), 2.11-1.78 (m, 9H), 1.71-0.98 (m, 33H), 1.37 (s, 6H), 1.33 (s, 6H), 1.05 (s, 3H), 0.95 (d, J=6.4 Hz, 3H), 0.89 (d, J=6.6 Hz, 3H), 0.88 (d, J=6.6 Hz, 3H), 0.73 (s, 3H).

### CF. Compound SA142: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride

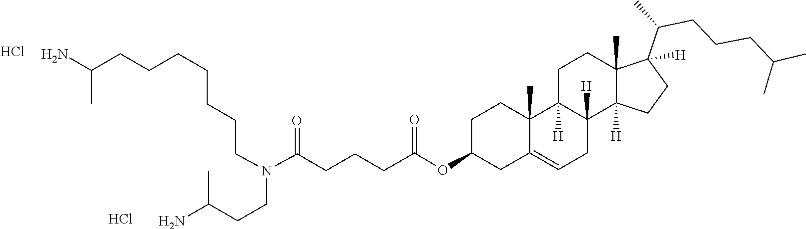

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-JH-cyclopenta[a]phenanthren-3-yl 5-((3-((tert-butoxycarbonyl)amino)butyl)(8-((tert-butoxycarbonyl)amino)nonyl)amino)-5-oxopentanoate

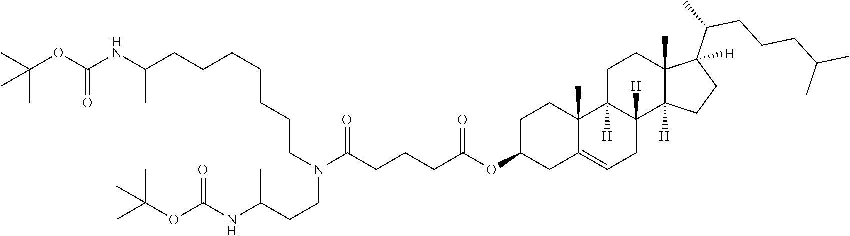

To a solution of 5-{[(1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl]oxy}-5-oxopentanoic acid (0.09 g, 0.18 mmol) in dry DCM (5 mL) stirring under nitrogen was added tert-butyl (9-((3-((tert-butoxycarbonyl)amino)butyl)amino)nonan-2-yl)carbamate (0.08 g, 0.18 mmol), dimethylaminopyridine (0.04 g, 0.35 mmol), and 1-ethyl-3-(3-dimethylaminopropyl)carbodiimide hydrochloride (0.07 g, 0.35 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate as a light yellow oil (0.13 g, 0.15 mmol, 82.9%). UPLC/ELSD: RT: 3.53 min. MS (ES): m/z (MH⁺) 913.4 for C₅₅H₉₇N₃O₇. ¹H NMR (300 MHz, CDCl₃) δ 5.35 (br. m, 1H), 4.59 (br. m, 2H), 4.38 (br. s, 1H), 3.59 (br. m, 2H), 3.22 (br. m, 4H), 2.32 (m, 6H), 1.93 (m, 4H), 1.81 (m, 3H), 1.50 (br. m, 10H), 1.42 (s, 18H), 1.27 (s, 14H), 1.09 (m, 12H), 0.99 (s, 6H), 0.90 (d, 4H, J=6 Hz), 0.83 (d, 6H, J=6 Hz), 0.66 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride

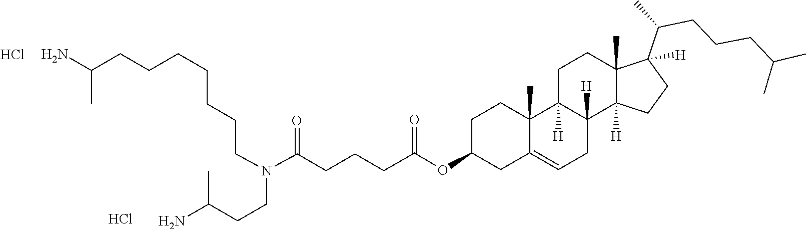

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate (0.13 g, 0.15 mmol) in DCM (3 mL) set stirring under nitrogen was added hydrochloric acid (4 N in dioxanes, 0.36 mL, 1.45 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride as a white solid (0.10 g, 0.12 mmol, 80.4%). UPLC/ELSD: RT=1.77 min. MS (ES): m/z (MH⁺) 713.3 for C₄₅H₈₃Cl₂N₃O₃. ¹H NMR (300 MHz, CDCl₃) δ 8.49 (br. m, 2H), 8.29 (br. m, 3H), 5.39 (s, 1H), 4.63 (br. m, 1H), 3.45 (br. m, 5H), 2.61 (br. m, 2H), 2.43 (br. m, 2H), 2.31 (d, 2H, J=9 Hz), 2.01 (br. m, 6H), 1.86 (br. m, 5H), 1.45 (br. m, 29H), 1.14 (br. m, 8H), 1.04 (s, 6H), 0.94 (d, 4H, J=6 Hz), 0.90 (d, 7H, J=6 Hz), 0.70 (s, 3H).

### CG. Compound SA144: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(8-aminononyl)carbamate dihydrochloride

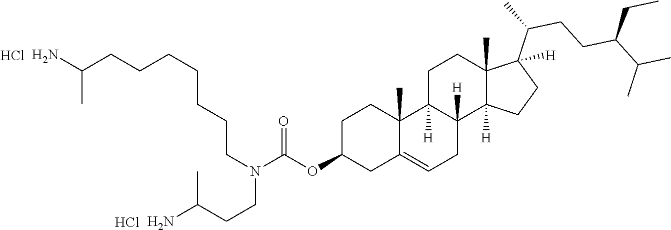

### Step 1: 8-bromo-N-methoxy-N-methyloctanamide

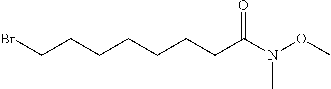

To a solution of 8-bromooctanoic acid (5.00 g, 22.41 mmol) in dry DCM (50 mL) stirring under nitrogen was added a solution of oxalyl chloride (16.81 mL, 33.62 mmol, 2M in DCM) dropwise. After the initial 3 mL of oxalyl chloride was added, catalytic dimethylformamide (0.17 mL, 2.24 mmol) was added, initiating gas formation seen by bubbling. Dropwise addition of the remainder of the oxalyl chloride solution followed. The reaction was allowed to proceed for 3 h at room temperature, and then the solution was concentrated in vacuo to a yellow oil. The residue was taken up in 30 mL DCM and added dropwise to a solution of N,O-dimethylhydroxylamine hydrochloride (2.69 g, 27.57 mmol) in 80 mL DCM, stirring under nitrogen. The solution was vented during addition as HCl gas was formed. The cloudy yellow reaction mixture was allowed to stir at room temperature overnight. Following, the mixture was diluted further with DCM, washed with water (1×30 mL), 1M HCl (1×30 mL), 1M NaOH (1×30 mL), and brine (1×30 mL), dried over sodium sulfate, filtered, and concentrated to give 8-bromo-N-methoxy-N-methyloctanamide as a clear yellow liquid used without further purification (5.87 g, 22.04 mmol, 98.4%). UPLC/ELSD: RT=0.67 min. MS (ES): m/z (MH⁺) 267.1 for C₁₀H₂₀BrNO₂. ¹H NMR (300 MHz, CDCl₃) δ: ppm 3.61 (s, 3H), 3.33 (t, 2H), 3.10 (s, 3H), 2.34 (t, 2H), 1.78 (qu, 2H), 1.56 (qu, 2H), 1.29 (br. m, 7H).

### Step 2: 9-bromononan-2-one

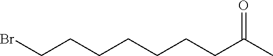

A solution of 8-bromo-N-methoxy-N-methyloctanamide (5.87 g, 22.04 mmol) in dry THF (100 mL) was set stirring under nitrogen and cooled to 0° C. Then, a solution of methylmagnesium bromide (11.02 ml, 33.06 mmol, 3M in diethyl ether) was added dropwise to the stirring mixture. The mixture was allowed to stir for 2.5 h at 0° C., and then allowed to gradually warm to room temperature for an additional 2 h. The mixture was then cooled again to 0° C., and the reaction was quenched with dropwise addition of hydrochloric acid (66.13 mL, 66.13 mmol, 1M). The mixture was allowed to continue stirring, gradually warming to room temperature over 30 minutes. The THF was removed under vacuum, and the mixture was extracted with EtOAc (3×50 mL). The combined organic phase was washed with brine (1×50 mL), dried over sodium sulfate, filtered, and concentrated in vacuo to an oil. The oil was taken up in DCM and purified on silica in hexanes with a 0-50% EtOAc gradient. Product-containing fractions were combined and concentrated to give 9-bromononan-2-one as a colorless oil (4.44 g, 20.08 mmol, 91.1%). UPLC/ELSD: RT=0.84 min. MS (ES): m/z (MH⁺) 222.1 for C₉H₁₇BrO. ¹H NMR (300 MHz, CDCl₃) δ: ppm 3.33 (t, 2H), 2.36 (t, 2H), 2.06 (s, 3H), 1.78 (qu, 2H), 1.50 (qu, 2H), 1.30 (br. m, 7H).

### Step 3: 9-bromononan-2-amine

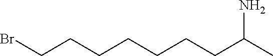

To a solution of 9-bromononan-2-one (3.44 g, 15.56 mmol) in dry MeOH (100 mL) was added ammonium acetate (10.79 g, 140.00 mmol) and sodium cyanoborohydride (1.27 g, 20.22 mmol). The solution was stirred vigorously for 36 h at room temperature. Following, the reaction was quenched with slow addition of HCl (100 mL, 2M). Then, 10M NaOH was added dropwise until the pH of the solution reached 11-12, measured qualitatively with pH paper. Then, the mixture was extracted with DCM (3×150 mL), and the combined organic phase was washed with brine (1×100 mL), dried over sodium sulfate, filtered, and concentrated to a yellow oil. The oil was taken up in DCM and purified on silica in DCM with a 0-50% (1:1 DCM/MeOH) gradient. Product-containing fractions were pooled and concentrated in vacuo to give 9-bromononan-2-amine as a colorless oil (1.23 g, 5.53 mmol, 35.6%). UPLC/ELSD: RT=0.89 min. MS (ES): m/z (MH⁺) 223.1 for C₉H₂₀BrN. ¹H NMR (300 MHz, CDCl₃) δ: ppm 3.29 (t, 2H), 2.82 (br. m, 2H), 2.66 (s, 3H), 1.74 (qu, 2H), 1.21 (br. m, 11H), 0.99 (d, 3H).

### Step 4: tert-butyl (9-bromononan-2-yl)carbamate

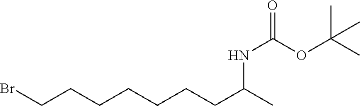 [SMILES: CC(CCCCCCCBr)NC(=O)OC(C)(C)C]

To a solution of 9-bromononan-2-amine (0.57 g, 2.54 mmol) in dry THF (10 mL) stirring under nitrogen at 0° C. was added di-tert-butyl dicarbonate (0.64 mL, 2.80 mmol). Then, triethylamine (0.39 mL, 2.80 mmol) was added dropwise, and the solution was allowed to gradually warm to room temperature and continue stirring overnight. Then solvent was removed under vacuum, and the resulting residue was taken up in DCM, washed with 5% aqueous HCl (1×15 mL) and water (1×15 mL), dried over sodium sulfate, filtered, and concentrated in vacuo to an oil. The oil was taken up in DCM and purified on silica in hexanes with a 0-20% EtOAc gradient. Product-containing fractions were pooled and concentrated in vacuo to give tert-butyl (9-bromononan-2-yl)carbamate as an oil (0.47 g, 1.47 mmol, 57.6%). UPLC/ELSD: RT=1.54 min. MS (ES): m/z (MH⁺) 323.3 for C₁₄H₂₈BrNO₂. ¹H NMR (300 MHz, CDCl₃) δ: ppm 4.35 (br. m, 1H), 3.60 (br. m, 1H), 3.37 (t, 2H), 1.84 (qu, 2H), 1.41 (br. s, 11H), 1.28 (br. m, 8H), 1.08 (d, 3H).

### Step 5: tert-butyl (9-((3-((tert-butoxycarbonyl)amino)butyl)amino)nonan-2-yl)carbamate

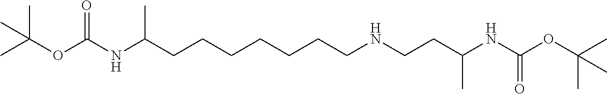 [SMILES: CC(CCCCCCCNCCC(C)NC(=O)OC(C)(C)C)NC(=O)OC(C)(C)C]

Both tert-butyl N-[4-(2-nitrobenzenesulfonamido)butan-2-yl]carbamate (0.88 g, 2.37 mmol) and tert-butyl (9-bromononan-2-yl)carbamate (0.76 g, 2.37 mmol) were dissolve in 20 mL dry DMF and set stirring under nitrogen. Then, potassium carbonate (1.96 g, 14.21 mmol) was added, and the solution was heated to 40° C. and allowed to stir overnight. The following morning, the mixture was cooled to room temperature, and benzyl bromide (0.17 mL, 1.42 mmol) was added. The solution stirred for 5 hours at room temperature. Then, thiophenol (0.727 mL, 7.10 mmol), potassium carbonate (0.98 g, 7.10 mmol), and an additional 10 mL dry DMF was added, and the reaction was allowed to stir for 2 days. Following, the salts were removed from the solution by centrifugation, and the supernatant was evaporated to a residue. The residue was taken up in 40 mL DCM and washed with water (2×10 mL) and brine (2×10 mL), dried over sodium sulfate, filtered, and concentrated to an oil. The oil was resuspended in DCM and purified on silica in DCM with a 0-50% (50:45:5 DCM/MeOH/aqueous NH₄OH) gradient. Product-containing fractions were pooled and concentrated to give tert-butyl (9-((3-((tert-butoxycarbonyl)amino)butyl)amino)nonan-2-yl)carbamate as a colorless oil (0.77 g, 1.80 mmol, 76.1%). UPLC/ELSD: RT=0.61 min. MS (ES): m/z (MH⁺) 430.6 for C₂₃H₄₇N₃O₄. ¹H NMR (300 MHz, CDCl₃) δ 4.94 (br. s, 1H), 4.33 (br. s, 1H), 3.62 (br. m, 2H), 2.61 (m, 4H), 1.66 (br. s, 1H), 1.45 (s, 21H), 1.29 (s, 9H), 1.14 (m, 6H).

### Step 6: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate

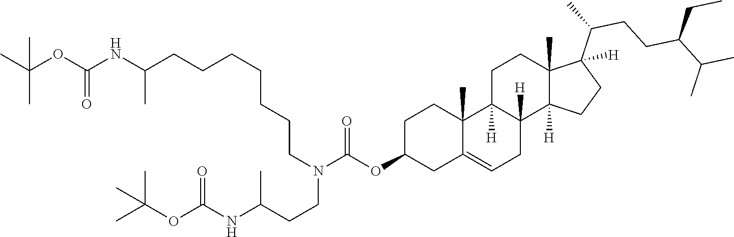

To a solution of tert-butyl (9-((3-((tert-butoxycarbonyl)amino)butyl)amino)nonan-2-yl)carbamate (0.11 g, 0.26 mmol) in dry toluene (5 mL) set stirring under nitrogen was added triethylamine (0.11 mL, 0.78 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.15 g, 0.26 mmol) was added, and the solution was heated to 90° C. and allowed to proceed for 2 days. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-30% EtOAc gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate as a light yellow oil (0.18 g, 0.21 mmol, 81.3%). UPLC/ELSD: RT=3.79 min. MS (ES): m/z (MH⁺) 871.4 for C₅₃H₉₅N₃O₆. ¹H NMR (300 MHz, CDCl₃) δ 5.07 (s, 1H), 4.14 (br. m, 3H), 3.33 (br. s, 2H), 2.91 (br. m, 4H), 2.07 (m, 2H), 1.66 (m, 6H), 1.26 (br. m, 10H), 1.14 (s, 21H), 0.98 (s, 15H), 0.83 (d, J=21.1 Hz, 12H), 0.73 (s, 6H), 0.65 (s, 5H), 0.56 (s, 9H), 0.39 (s, 3H).

### Step 7: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(8-aminononyl)carbamate dihydrochloride

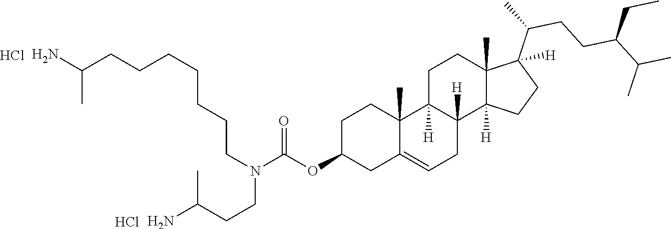

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate (0.18 g, 0.21 mmol) in DCM (3 mL) set stirring under nitrogen was added hydrochloric acid (4 N in dioxanes, 0.53 mL, 2.11 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(8-aminononyl)carbamate dihydrochloride as a white solid (0.14 g, 0.18 mmol, 83.4%). UPLC/ELSD: RT=1.89 min. MS (ES): m/z (MH⁺) 671.3 for C₄₃H₈₁Cl₂N₃O₂. ¹H NMR (300 MHz, CDCl₃) δ 8.52 (br. m, 3H), 8.33 (br. m, 3H), 5.40 (br. s, 1H), 4.52 (br. s, 1H), 3.33 (br. m, 6H), 2.37 (m, 2H), 2.01 (br. m, 7H), 1.34 (br. m, 31H), 1.18 (dd, J=13.1, 7.6 Hz, 4H), 1.11 (s, 2H), 1.04 (br. m, 4H), 0.93 (br. s, 4H), 0.85 (q, 8H), 0.68 (s, 3H).

### CH. Compound SA145: N-(3-amino-3-methylbutyl)-N-(4-amino-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

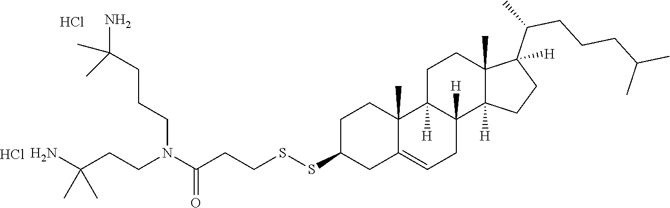

### Step 1: tert-butyl (4-(N-(4-((tert-butoxycarbonyl)amino)-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylbutan-2-yl)carbamate

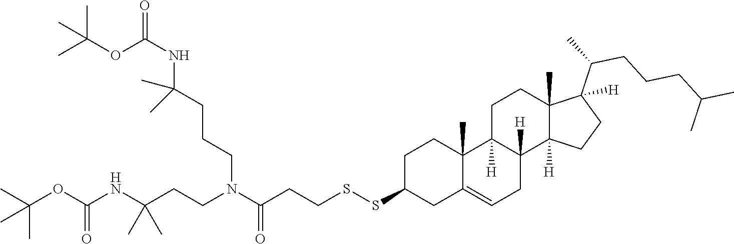

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.11 g, 0.22 mmol) in dry DCM (5 mL) stirring under nitrogen was added tert-butyl (5-((3-((tert-butoxycarbonyl)amino)-3-methylbutyl)amino)-2-methylpentan-2-yl)carbamate (0.09 g, 0.22 mmol), dimethylaminopyridine (0.06 g, 0.45 mmol), and 1-ethyl-3-(3-dimethylaminopropyl)carbodiimide hydrochloride (0.09 g, 0.45 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give tert-butyl (4-(N-(4-((tert-butoxycarbonyl)amino)-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylbutan-2-yl)carbamate as a light yellow oil (0.17 g, 0.19 mmol, 86.7%). UPLC/ELSD: RT: 3.70 min. MS (ES): m/z (MH⁺) 891.4 for C₅₁H₉₁N₃O₅S2. ¹H NMR (300 MHz, CDCl₃) δ 5.27 (br. s, 1H), 4.68 (s, 1H), 4.41 (br. s, 1H), 4.03 (q, 1H), 3.20 (br. m, 4H), 2.87 (br. m, 2H), 2.62 (br. m, 3H), 2.26 (br. m, 2H), 1.96 (br. m, 8H), 1.50 (br. m, 9H), 1.37 (s, 19H), 1.28 (br. m, 3H), 1.20 (m, 14H), 1.04 (br. m, 6H), 0.92 (s, 5H), 0.85 (d, 4H, J=6 Hz), 0.80 (d, 6H, J=6 Hz), 0.60 (s, 3H).

### Step 2: N-(3-amino-3-methylbutyl)-N-(4-amino-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

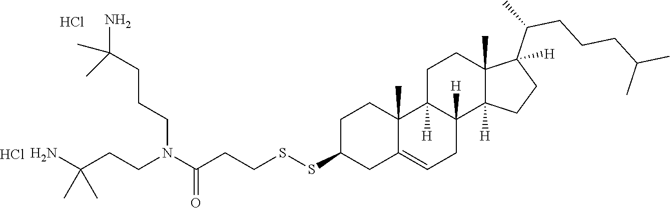

To a solution of tert-butyl (4-(N-(4-((tert-butoxycarbonyl)amino)-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylbutan-2-yl)carbamate (0.17 g, 0.19 mmol) in DCM (5 mL) set stirring under nitrogen was added hydrochloric acid (4 M in dioxanes, 0.49 mL, 1.94 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give N-(3-amino-3-methylbutyl)-N-(4-amino-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride as a white solid (0.13 g, 0.17 mmol, 85.9%). UPLC/ELSD: RT=2.12 min. MS (ES): m/z (MH⁺) 691.3 for C₄₁H₇₇Cl₂N₃₀S₂. ¹H NMR (300 MHz, MeOD) δ 5.39 (br. s, 1H), 3.48 (br. m, 4H), 3.33 (br. s, 2H), 2.98 (br. m, 2H), 2.86 (br. m, 2H), 2.67 (br. m, 1H), 2.37 (d, 2H, J=6 Hz), 1.97 (br. m, 7H), 1.66 (br. m, 12H), 1.46 (s, 4H), 1.41 (s, 13H), 1.31 (s, 3H), 1.17 (br. m, 8H), 1.05 (s, 5H), 0.96 (d, 4H, J=6 Hz), 0.90 (d, 8H, J=6 Hz), 0.75 (s, 3H).

### CI. Compound SA149: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride

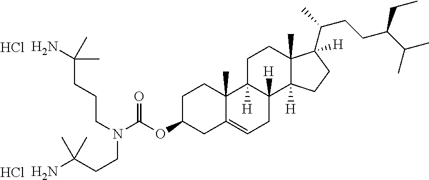

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpent 1carbamate

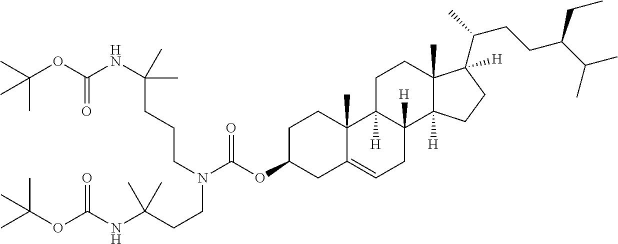

To a solution of tert-butyl (5-((3-((tert-butoxycarbonyl)amino)-3-methylbutyl)amino)-2-methylpentan-2-yl)carbamate (0.09 g, 0.22 mmol) in dry toluene (5 mL) set stirring under nitrogen was added triethylamine (0.07 mL, 0.09 mmol). Then, (1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,10H,11H-cyclopenta[a]phenanthren-7-yl 4-nitrophenyl carbonate (0.13 g, 0.22 mmol) was added, and the solution was heated to 90° C. and allowed to proceed for 2 days. Then, the reaction mixture was allowed to cool to room temperature, diluted with toluene, and washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate as a light yellow oil (0.14 g, 0.17 mmol, 74.7%). UPLC/ELSD: RT=3.78 min. MS (ES): m/z (MH⁺) 843.4 for C₅₁H₉₁N₃O₆. ¹H NMR (300 MHz, CDCl₃) δ 5.30 (br. s, 1H), 4.42 (br. m, 3H), 3.12 (br. s, 4H), 2.28 (br. m, 2H), 1.80 (br. m, 7H), 1.52 (br. m, 11H), 1.35 (s, 18H), 1.20 (s, 18H), 1.08 (br. m, 5H), 0.94 (s, 6H), 0.86 (d, 5H, J=6 Hz), 0.75 (q, 9H), 0.61 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride

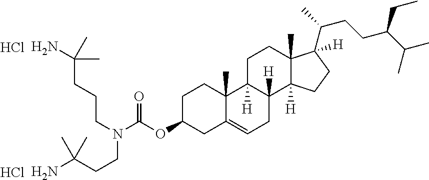

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate (0.14 g, 0.17 mmol) in DCM (5 mL) set stirring under nitrogen was added hydrochloric acid (4 M in dioxanes, 0.42 mL, 1.67 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride as a white solid (0.08 g, 0.10 mmol, 61.2%). UPLC/ELSD: RT=2.07 min. MS (ES): m/z (MH⁺) 643.3 for C₄₁H₇₇C₂N₃O₂. ¹H NMR (300 MHz, MeOD) δ 5.44 (br. s, 1H), 4.47 (br. m, 1H), 3.34 (br. m, 7H), 2.40 (br. m, 2H), 1.97 (br. m, 7H), 1.66 (br. m, 11H), 1.37 (d, 14H, J=6 Hz), 1.20 (br. m, 8H), 1.08 (s, 5H), 0.99 (d, 5H, J=6 Hz), 0.87 (q, 8H), 0.75 (s, 3H).

### CJ. Compound SA151: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(8-aminononyl)carbamate dihydrochloride

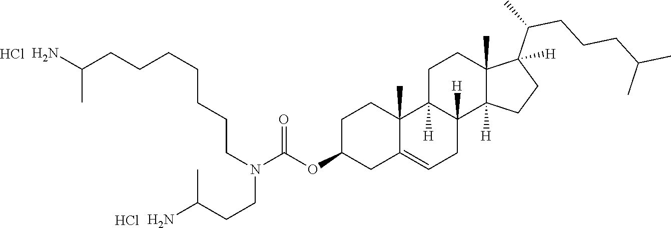

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-JH-cyclopenta[a]phenanthren-3-yl (3-((tert-butoxycarbonyl)amino)butyl)(8-((tert-butoxycarbonyl)amino)nonyl)carbamate

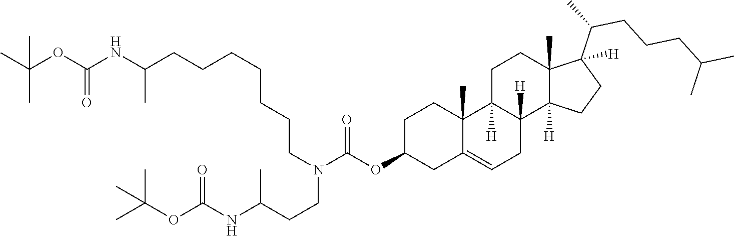

To a solution of tert-butyl (9-((3-((tert-butoxycarbonyl)amino)butyl)amino)nonan-2-yl)carbamate (0.12 g, 0.27 mmol) in dry toluene (5 mL) set stirring under nitrogen was added triethylamine (0.12 mL, 0.82 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.15 g, 0.27 mmol) was added, and the solution was heated to 90° C. and allowed to proceed for 2 days. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate as a light yellow oil (0.17 g, 0.21 mmol, 75.6%). UPLC/ELSD: RT=3.70 min. MS (ES): m/z (MH⁺) 843.4 for C₅₁H₉₁N₃O₆. ¹H NMR (300 MHz, CDCl₃) δ 5.35 (br. s, 1H), 4.46 (br. m, 3H), 3.59 (br. m, 2H), 3.19 (br. m, 4H), 2.29 (m, 2H), 2.01 (m, 6H), 1.59 (br. m, 10H), 1.40 (s, 20H), 1.25 (br. m, 15H), 1.10 (q, 12H), 0.99 (s, 6H), 0.90 (d, 4H, J=6 Hz), 0.83 (d, 6H, J=6 Hz), 0.65 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(8-aminononyl)carbamate dihydrochloride

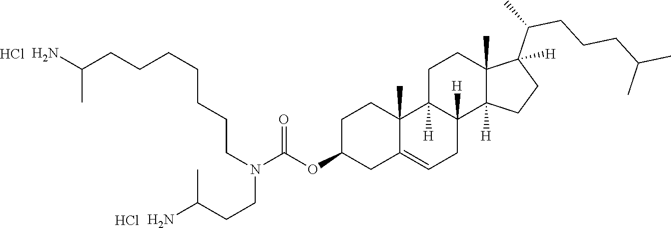

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate (0.17 g, 0.21 mmol) in DCM (3 mL) set stirring under nitrogen was added hydrochloric acid (4 N in dioxanes, 0.52 mL, 2.05 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(8-aminononyl)carbamate dihydrochloride as a white solid (0.15 g, 0.20 mmol, 95.7%). UPLC/ELSD: RT=1.83 min. MS (ES): m/z (MH⁺) 643.3 for C₄₁H₇₇Cl₂N₃O₂. ¹H NMR (301 MHz, CDCl₃) δ 8.51 (br. s, 3H), 8.32 (br. s, 3H), 5.39 (br. m, 1H), 4.50 (br. m, 1H), 3.34 (br. m, 6H), 2.37 (m, 2H), 2.01 (br. m, 7H), 1.45 (br. m, 29H), 1.11 (br. m, 8H), 1.04 (s, 4H), 0.93 (d, 3H, J=6 Hz, 3H), 0.90 (d, 3H, J=6 Hz, 6H), 0.70 (s, 3H).

### CK. Compound SA152: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride

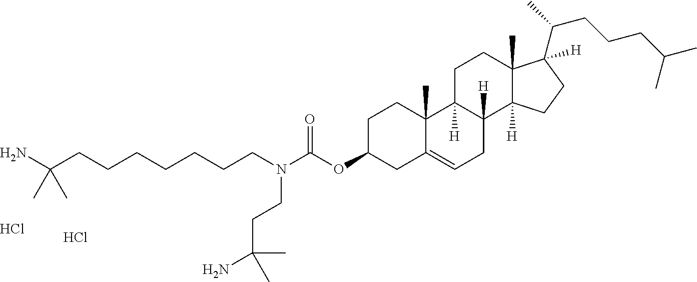

### Step 1: methyl 9-chloro-2,2-dimethylnonanoate

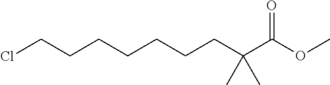

To a solution of THF (30 mL) and lithium diisopropylamide (19 mL, 2.0 M in THF) cooled to −78° C. was added methyl isobutyrate (3.0 mL, 26 mmol). The reaction mixture stirred at 0° C. for 50 min then was cooled to −78° C. 1-Bromo-7-chloroheptane (4.2 mL, 27 mmol) was added dropwise. The reaction mixture was stirred while slowly coming to room temperature and was monitored by TLC. At 20 hours, the reaction mixture was cooled to 0° C., and then aq. 1 N HCl (30 mL) was added dropwise. The biphasic mixture was separated, and the aqueous layer was extracted with EtOAc (2×30 mL). The combined organics were washed with brine, dried over Na₂SO₄, and concentrated to afford methyl 9-chloro-2,2-dimethylnonanoate (6.205 g, quant.) as an amber oil. Material was carried forward as is. 1H NMR (300 MHz, CDCl₃): δ 3.65 (s, 3H), 3.52 (t, J=6.7 Hz, 2H), 1.83-1.63 (m, 2H), 1.63-1.17 (m, 10H), 1.15 (s, 6H).

### Step 2: 9-chloro-2,2-dimethylnonanoic acid

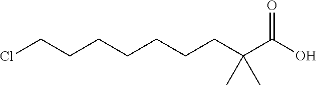

A mixture of methyl 9-chloro-2,2-dimethylnonanoate (6.2 g, 26 mmol), THF (60 mL), MeOH (45 mL), and aq. 10% NaOH (31 mL, 78 mmol) was stirred at 50° C. The reaction was monitored by TLC. At 23 hours, the reaction mixture was concentrated to remove volatile organics. The residue was taken up in water (70 mL), washed with MTBE (2×50 mL), and then acidified to pH ˜1 with aq. 2 N HCl. The aqueous was extracted with EtOAc (3×50 mL), dried over Na₂SO₄, and then concentrated to afford 9-chloro-2,2-dimethylnonanoic acid (4.997 g, 22.64 mmol, 85.7%) as an amber oil. UPLC/ELSD: RT=1.00 min. MS (ES): m/z=174.98 (M—CO₂H)⁺ for C₁₁H₂₁ClO₂. ¹H NMR (300 MHz, CDCl3): δ 9.71 (br. s, 1H), 3.53 (t, J=6.7 Hz, 2H), 1.88-1.66 (m, 2H), 1.62-1.22 (m, 10H), 1.19 (s, 6H).

### Step 3: (4-methoxyphenyl)methyl N-(9-chloro-2-methylnonan-2-yl)carbamate

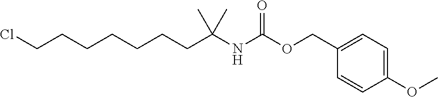 [SMILES: COc1ccc(COC(=O)NC(C)(C)CCCCCCCCl)cc1]

To a stirred solution of 9-chloro-2,2-dimethylnonanoic acid (2.00 g, 9.06 mmol) and triethylamine (1.8 mL, 13 mmol) in PhMe (30 mL) was added diphenylphosphoryl azide (2.4 mL, 11 mmol). The reaction mixture stirred at room temperature for 1.25 hours, then was stirred at 80° C. Gas evolution occurred. At 2 hours, the reaction mixture was cooled to room temperature, then washed with 5% aq. NaHCO₃ soln. (2×), water, and brine. The organics were dried over Na₂SO₄, and then 4-methoxybenzyl alcohol (2.2 mL, 18 mmol) and 1,8-diazabicyclo[5.4.0]undec-7-ene (2.8 mL, 19 mmol) were added sequentially. The reaction mixture was stirred at 80° C. and was monitored by LCMS. At 18 hours, the reaction mixture was cooled to room temperature, diluted with EtOAc (150 mL), washed with 5% aq. citric acid (2×), water, and brine, dried over Na₂SO₄, and concentrated. The crude material was purified via silica gel chromatography (0-30% EtOAc in hexanes) to afford (4-methoxyphenyl)methyl N-(9-chloro-2-methylnonan-2-yl)carbamate (1.613 g, 4.532 mmol, 50.0%) as a clear oil. UPLC/ELSD: RT=1.70 min. MS (ES): m/z=378.33 (M+Na)⁺ for C₁₉H₃₀ClNO₃. ¹H NMR (300 MHz, CDCl3): δ 7.29 (d, J=8.3 Hz, 2H), 6.88 (d, J=8.1 Hz, 2H), 4.98 (s, 2H), 4.58 (s, 1H), 3.81 (s, 3H), 3.53 (t, J=6.7 Hz, 2H), 1.84-1.69 (m, 2H), 1.68-1.16 (m, 16H).

### Step 4: tert-butyl N-(4-{N-[8-({[(4-methoxyphenyl)methoxy]carbonyl}amino)-8-methylnonyl]-2-nitrobenzenesulfonamido}-2-methylbutan-2-yl)carbamate

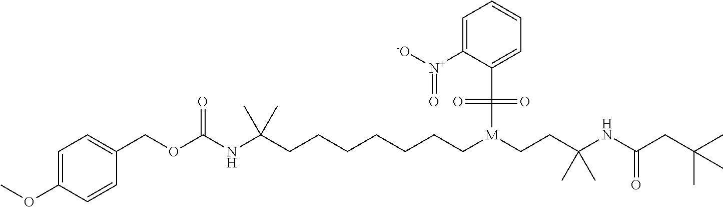

Tert-butyl N-[2-methyl-4-(2-nitrobenzenesulfonamido)butan-2-yl]carbamate (0.907 g, 2.34 mmol), (4-methoxyphenyl)methyl N-(9-chloro-2-methylnonan-2-yl)carbamate (0.700 g, 1.97 mmol), potassium carbonate (0.544 g, 3.93 mmol), potassium iodide (0.164 g, 0.983 mmol) and propionitrile (10.5 mL) were combined in a sealed tube. The reaction mixture was heated at 150° C. via microwave irradiation while stirring and was monitored by LCMS. At 12 hours, the reaction mixture was cooled to room temperature and filtered rinsing with ACN, and the filtrate was concentrated. The residue was taken up in EtOAc (100 mL), then washed with 5% aq. NaHCO₃ soln. and brine, dried over Na₂SO₄, and concentrated. The crude material was purified via silica gel chromatography (20-50% EtOAc in hexanes) to afford tert-butyl N-(4-{N-[8-({[(4-methoxyphenyl)methoxy]carbonyl}amino)-8-methylnonyl]-2-nitrobenzenesulfonamido}-2-methylbutan-2-yl)carbamate (1.267 g, 1.792 mmol, 91.1%) as a yellow oil. UPLC/ELSD: RT=2.00 min. MS (ES): m/z=607.64 [(M+H)—(CH₃)₂C═CH₂— CO₂]⁺ for C₃₅H₅₄N₄O₉S. ¹H NMR (300 MHz, CDCl₃) δ 8.06-7.93 (m, 1H), 7.77-7.52 (m, 3H), 7.37-7.20 (m, 2H), 6.97-6.78 (m, 2H), 4.97 (s, 2H), 4.59 (s, 1H), 4.39 (s, 1H), 3.80 (s, 3H), 3.41-3.20 (m, 4H), 2.00-1.84 (m, 2H), 1.66-1.10 (m, 33H).

### Step 5: tert-butyl N-(4-{[8-({[(4-methoxyphenyl)methoxy]carbonyl}amino)-8-methylnonyl]amino}-2-methylbutan-2-yl)carbamate

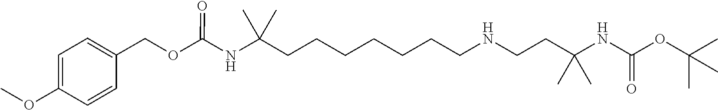 [SMILES: COc1ccc(COC(=O)NC(C)(C)CCCCCCCNCCC(C)(C)NC(=O)OC(C)(C)C)cc1]

To a mixture of tert-butyl N-(4-{N-[8-({[(4-methoxyphenyl)methoxy]carbonyl}amino)-8-methylnonyl]-2-nitrobenzenesulfonamido}-2-methylbutan-2-yl)carbamate (1.258 g, 1.780 mmol) and potassium carbonate (0.738 g, 5.34 mmol) in DMF (19 mL) was added thiophenol (0.33 mL, 3.2 mmol). The reaction mixture was stirred at room temperature and was monitored by LCMS. At 2 hours, the reaction mixture was filtered rinsing with EtOAc. The filtrate was diluted to 125 mL with EtOAc, washed with 5% aq. NaHCO₃ soln., water (3×), and brine, dried over Na₂SO₄, and concentrated. The crude material was purified via silica gel chromatography (0-16% (5% conc. aq. NH₄OH in MeOH) in DCM) to afford tert-butyl N-(4-{[8-({[(4-methoxyphenyl)methoxy]carbonyl}amino)-8-methylnonyl]amino}-2-methylbutan-2-yl)carbamate (0.699 g, 1.34 mmol, 75.3%) as a yellow oil. UPLC/ELSD: RT=0.89 min. MS (ES): m/z=522.74 (M+H)⁺ for C₂₉H₅₁N₃O₅. ¹H NMR (300 MHz, CDCl₃) δ 7.33-7.27 (m, 2H), 6.91-6.84 (m, 2H), 5.61 (s, 1H), 4.97 (s, 2H), 4.59 (s, 1H), 3.80 (s, 3H), 2.75 (t, J=7.2 Hz, 2H), 2.64 (t, J=7.3 Hz, 2H), 1.81 (t, J=7.2 Hz, 2H), 1.70-1.13 (m, 33H).

### Step 6: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-JH-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate

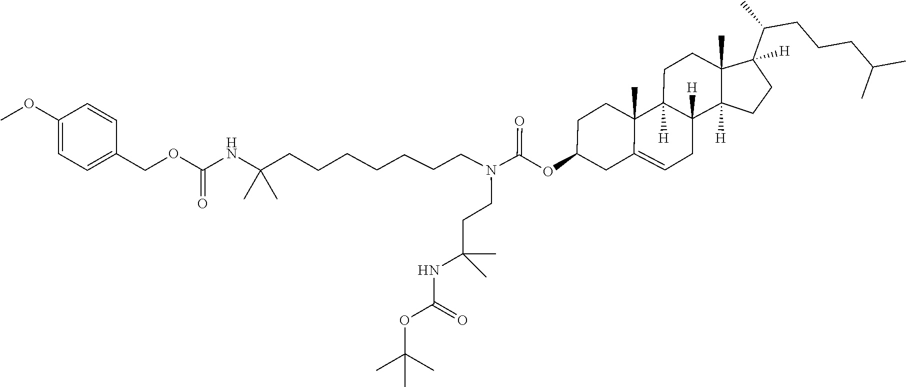

Cholesterol 4-nitrophenyl carbonate (0.150 g, 0.272 mmol), tert-butyl N-(4-{[8-({[(4-methoxyphenyl)methoxy]carbonyl}amino)-8-methylnonyl]amino}-2-methylbutan-2-yl)carbamate (0.184 g, 0.353 mmol), and triethylamine (0.12 mL, 0.86 mmol) were combined in PhMe (2.05 mL). The reaction mixture was stirred at 90° C. and was monitored by LCMS. At 20 hours, the reaction mixture was cooled to room temperature, diluted with DCM (30 mL), and then washed with 5% aq. NaHCO₃ soln. (3×). The organics were passed through a hydrophobic frit, dried over Na₂SO₄, and concentrated. The crude material was purified via silica gel chromatography (0-30% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate (0.227 g, 0.243 mmol, 89.4%) as a clear oil. UPLC/ELSD: RT=3.79 min. MS (ES): m/z=935.72 (M+H)⁺ for C₅₇H₉₅N₃O₇. ¹H NMR (300 MHz, CDCl₃) δ 7.34-7.24 (m, 2H), 6.95-6.83 (m, 2H), 5.42-5.32 (m, 1H), 4.97 (s, 2H), 4.64-4.32 (m, 3H), 3.80 (s, 3H), 3.33-3.07 (m, 4H), 2.48-2.21 (m, 2H), 2.12-0.94 (m, 61H), 1.02 (s, 3H), 0.91 (d, J=6.4 Hz, 3H), 0.87 (d, J=6.6 Hz, 6H), 0.68 (s, 3H).

### Step 7: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride

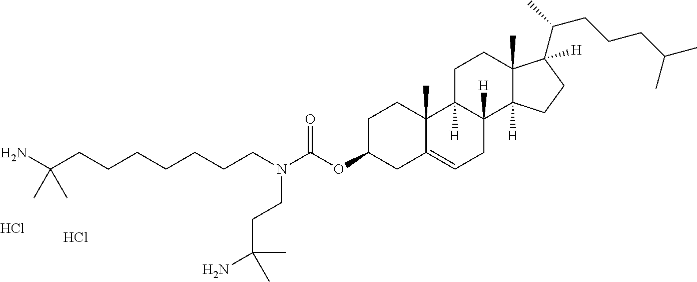

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate (0.222 g, 0.238 mmol) in DCM (2.6 mL) was added 4 N HCl in dioxane (0.43 mL). The reaction mixture was stirred at room temperature and was monitored by LCMS. At 18 hours, hexanes (30 mL) was added, and the mixture was centrifuged (10,000×g for 30 min). The supernatant was decanted, the solids were suspended in hexanes (30 mL), and the suspension was centrifuged (10,000×g for 30 min). The supernatant was decanted, and the solids were dried under reduced pressure to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride (0.125 g, 0.163 mmol, 68.8%) as a white solid. UPLC/ELSD: RT=1.87 min. MS (ES): m/z=335.74 (M+2H)²⁺ for C₄₃H₇₉N₃O₂. ¹H NMR (300 MHz, DMSO) δ 8.29-7.87 (m, 6H), 5.41-5.26 (m, 1H), 4.39-4.22 (m, 1H), 3.29-3.06 (m, 4H), 2.36-2.11 (m, 2H), 2.09-0.90 (m, 52H), 0.98 (s, 3H), 0.89 (d, J=6.2 Hz, 3H), 0.84 (d, J=6.6 Hz, 6H), 0.65 (s, 3H).

### CL. Compound SA153: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride

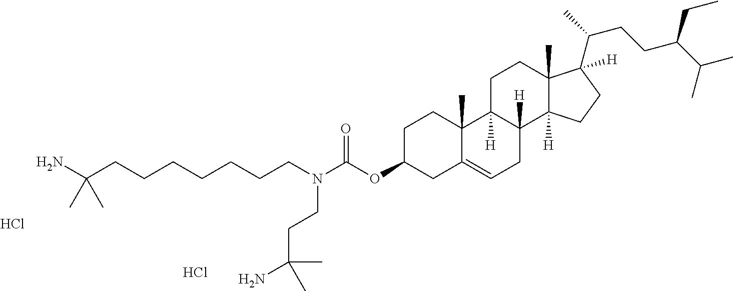

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate

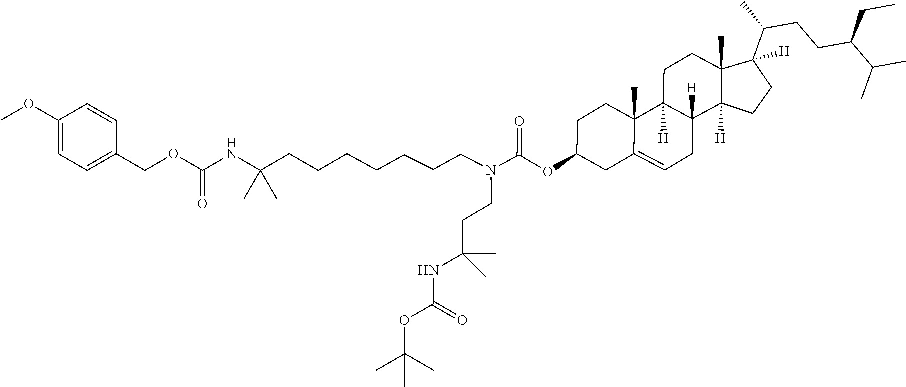

Sitosterol 4-nitrophenyl carbonate (0.175 g, 0.302 mmol), tert-butyl N-(4-{[8-({[(4-methoxyphenyl)methoxy]carbonyl}amino)-8-methylnonyl]amino}-2-methylbutan-2-yl)carbamate (0.205 g, 0.392 mmol), and triethylamine (0.13 mL, 0.93 mmol) were combined in PhMe (2.3 mL). The reaction mixture was stirred at 90° C. and was monitored by LCMS. At 20 hours, the reaction mixture was cooled to room temperature, diluted with DCM (30 mL), and then washed with 5% aq. NaHCO₃ soln. (3×). The organics were passed through a hydrophobic frit, dried over Na₂SO₄, and concentrated. The crude material was purified via silica gel chromatography (0-30% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate (0.252 g, 0.262 mmol, 86.8%) as a clear oil. UPLC/ELSD: RT=3.89 min. MS (ES): m/z=963.23 (M+H)⁺ for C₅₉H₉₉N₃O₇. ¹H NMR (300 MHz, CDCl₃) δ 7.32-7.24 (m, 2H), 6.94-6.85 (m, 2H), 5.42-5.32 (m, 1H), 4.97 (s, 2H), 4.66-4.40 (m, 3H), 3.81 (s, 3H), 3.33-3.08 (m, 4H), 2.47-2.20 (m, 2H), 2.13-0.77 (m, 71H), 1.02 (s, 3H), 0.92 (d, J=6.3 Hz, 3H), 0.68 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride

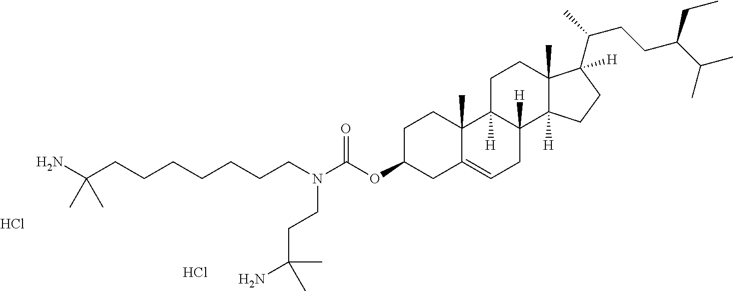

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate (0.248 g, 0.258 mmol) in DCM (2.6 mL) was added 4 N HCl in dioxane (0.46 mL). The reaction mixture was stirred at room temperature and was monitored by LCMS. At 18 hours, hexanes (30 mL) was added, and the mixture was centrifuged (10,000×g for 30 min). The supernatant was decanted, the solids suspended in hexanes (30 mL), and the suspension was centrifuged (10,000×g for 30 min). The supernatant was decanted, and the solids were dried under reduced pressure to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride (0.107 g, 0.130 mmol, 50.6%) as a white solid. UPLC/ELSD: RT=3.89 min. MS (ES): m/z=370.68 [(M+2H)+CH₃CN]²⁺ for C₄₅H₈₃N₃O₂. ¹H NMR (300 MHz, DMSO) δ 8.34-7.91 (m, 6H), 5.39-5.29 (m, 1H), 4.41-4.21 (m, 1H), 3.30-3.07 (m, 4H), 2.37-2.16 (m, 2H), 2.05-0.74 (m, 62H), 0.98 (s, 3H), 0.90 (d, J=6.3 Hz, 3H), 0.65 (s, 3H).

### CM. Compound SA154: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride

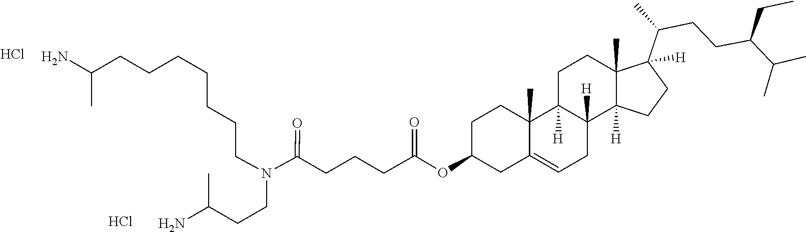

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate

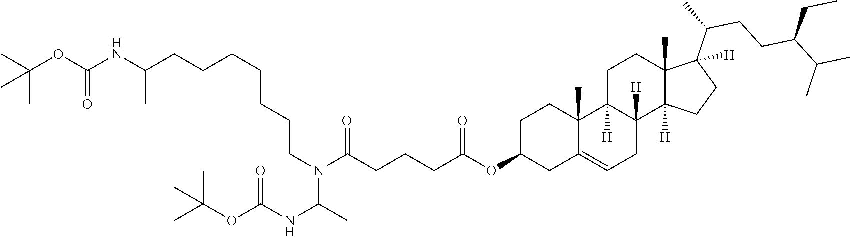

To a solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.09 g, 0.18 mmol) in dry DCM (5 mL) stirring under nitrogen was added tert-butyl (9-((3-((tert-butoxycarbonyl)amino)butyl)amino)nonan-2-yl)carbamate (0.08 g, 0.18 mmol), dimethylaminopyridine (0.04 g, 0.35 mmol), and 1-ethyl-3-(3-dimethylaminopropyl)carbodiimide hydrochloride (0.07 g, 0.35 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate as a light yellow oil (0.09 g, 0.10 mmol, 57.3%). UPLC/ELSD: RT: 3.63 min. MS (ES): m/z (MH⁺) 941.4 for C₅₇H₁₀₁N₃O₇. ¹H NMR (300 MHz, CDCl₃) δ 5.35 (br. s, 1H), 4.60 (br. m, 2H), 4.34 (br. m, 1H), 3.63 (br. m, 3H), 3.20 (br. m, 3H), 2.35 (m, 6H), 1.94 (m, 4H), 1.83 (br. m, 3H), 1.55 (br. m, 10H), 1.43 (s, 18H), 1.28 (br. m, 16H), 1.11 (m, 12H), 1.01 (s, 5H), 0.92 (d, 5H, J=6 Hz), 0.82 (q, 10H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride

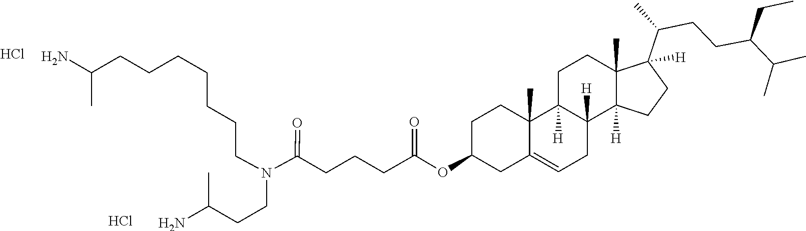

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate (0.09 g, 0.10 mmol) in DCM (3 mL) set stirring under nitrogen was added hydrochloric acid (4 N in dioxanes, 0.25 mL, 1.00 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride as a white solid (0.06 g, 0.06 mmol, 63.4%). UPLC/ELSD: RT=1.96 min. MS (ES): m/z (MH⁺) 741.3 for C₄₇H₈₇Cl₂N₃O₃. ¹H NMR (300 MHz, CDCl₃) δ 8.41 (m, 6H), 5.38 (br. s, 1H), 4.83 (br. s, 1H), 4.61 (br. m, 1H), 3.56 (br. m, 6H), 2.50 (br. m, 2H), 2.41 (br. m, 2H), 2.31 (d, 2H, J=9 Hz), 2.01 (br. m, 6H), 1.86 (br. m, 3H), 1.62 (br. m, 9H), 1.46 (br. m, 16H), 1.19 (br. m, 11H), 1.04 (s, 5H), 0.96 (d, 5H, J=6 Hz), 0.85 (q, 10H), 0.70 (s, 3H).

### CN. Compound SA155: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride

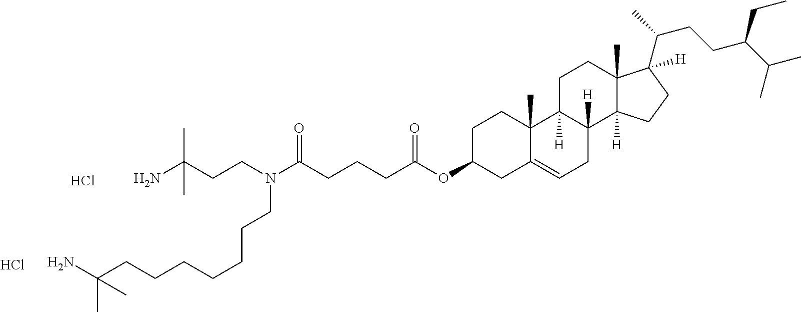

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate

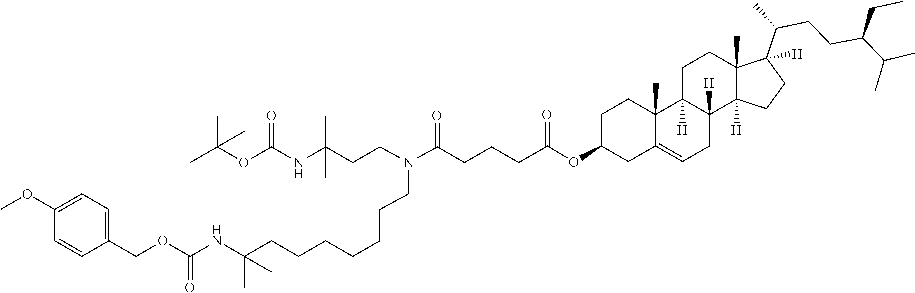

To a stirred solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.100 g, 0.189 mmol), tert-butyl N-(4-{[8-({[(4-methoxyphenyl)methoxy]carbonyl}amino)-8-methylnonyl]amino}-2-methylbutan-2-yl)carbamate (0.099 g, 0.19 mmol), and DMAP (0.046 g, 0.38 mmol) in DCM (2.0 mL) was added 1-ethyl-3-(3-dimethylaminopropyl)carbodiimide hydrochloride (0.073 g, 0.38 mmol). The reaction mixture was stirred at room temperature and was monitored by LCMS. At 15 hours, the reaction mixture was diluted with DCM (15 mL) and washed with 5% aq. NaHCO₃ soln. The aqueous was extracted with DCM (15 mL). The combined organics were passed through a hydrophobic frit, dried over Na₂SO₄, and concentrated. The crude material was purified via silica gel chromatography (0-50% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate (0.121 g, 0.117 mmol, 62.0%) as a clear oil. UPLC/ELSD: RT=3.77 min. MS (ES): m/z=1034.04 (M+H)⁺ for C₆₃H₁₀₅N₃O₈. ¹H NMR (300 MHz, CDCl₃) δ 7.33-7.27 (m, 2H), 6.92-6.84 (m, 2H), 5.42-5.31 (m, 1H), 4.97 (s, 2H), 4.77-4.30 (m, 3H), 3.81 (s, 3H), 3.36-3.14 (m, 4H), 2.43-2.24 (m, 6H), 2.16-0.76 (m, 73H), 1.01 (s, 3H), 0.92 (d, J=6.5 Hz, 3H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride

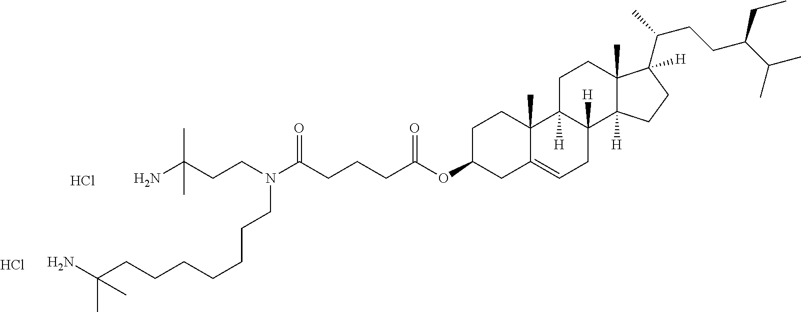

To a stirred solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate (0.116 g, 0.112 mmol) in DCM (2.0 mL) was added 4 N HCl in dioxane (0.20 mL). The reaction mixture was stirred at room temperature and was monitored by LCMS. At 17 hours, 4 N HCl in dioxane (0.10 mL) was added. At 22 hours, MTBE (20 mL) was added, and the reaction mixture was held at 4° C. overnight. The suspension was centrifuged (10,000×g for 30 min at 4° C.). The supernatant was decanted. The solids were suspended in MTBE and concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride (0.073 g, 0.080 mmol, 71.4%) as a white solid. UPLC/ELSD: RT=2.38 min. MS (ES): m/z=385.65 (M+2H)²⁺ for C₄₉H₈₉N₃O₃. ¹H NMR (300 MHz, MeOD) δ 5.43-5.32 (m, 1H), 4.62-4.47 (m, 1H), 3.50-3.33 (m, 4H), 2.51-2.23 (m, 6H), 2.13-1.77 (m, 9H), 1.76-0.77 (m, 43H), 1.37 (s, 6H), 1.33 (s, 6H), 1.05 (s, 3H), 0.96 (d, J=6.4 Hz, 3H), 0.75-0.70 (m, 3H).

### CO. Compound SA156: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-aminobutyl)amino)-5-oxopentanoate dihydrochloride

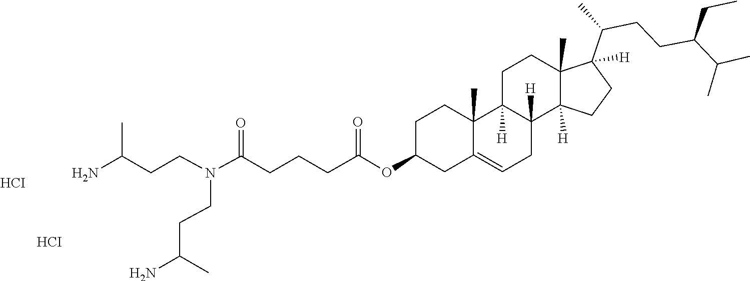

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-((tert-butoxycarbonyl)amino)butyl)amino)-5-oxopentanoate

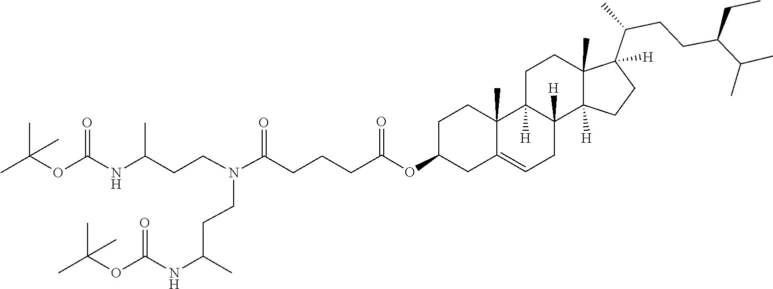

To a stirred solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.100 g, 0.189 mmol), tert-butyl N-[4-({3-[(tert-butoxycarbonyl)amino]butyl}amino)butan-2-yl]carbamate (0.075 g, 0.21 mmol), and DMAP (0.051 g, 0.42 mmol) in DCM (2.0 mL) was added 1-ethyl-3-(3-dimethylaminopropyl)carbodiimide hydrochloride (0.073 g, 0.38 mmol). The reaction mixture was stirred at room temperature and was monitored by LCMS. At 17 hours, the reaction mixture was diluted with DCM (10 mL), then washed with 5% aq. NaHCO₃ soln. The aqueous was extracted with DCM (10 mL). The combined organics were passed through a hydrophobic frit, dried over Na₂SO₄, and concentrated. The crude material was purified via silica gel chromatography (20-65% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-11H-cyclopenta[a]phenanthren-3-yl 5-(bis(3-((tert-butoxycarbonyl)amino)butyl)amino)-5-oxopentanoate (0.097 g, 0.11 mmol, 58.9%) as a white foam. UPLC/ELSD: RT=3.57 min. MS (ES): m/z=871.11 (M+H)⁺ for C₅₂H₉₁N₃O₇. ¹H NMR (300 MHz, CDCl3) δ 5.48-5.30 (m, 1H), 4.77-4.42 (m, 3H), 3.79-3.07 (m, 6H), 2.52-2.19 (m, 6H), 2.14-0.76 (m, 66H), 1.01 (s, 3H), 0.92 (d, J=6.4 Hz, 3H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-aminobutyl)amino)-5-oxopentanoate dihydrochloride

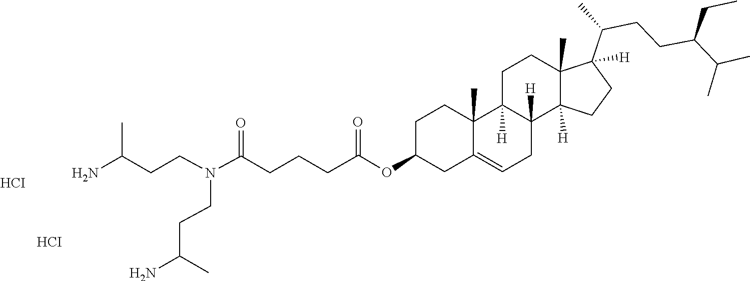

To a stirred solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-((tert-butoxycarbonyl)amino)butyl)amino)-5-oxopentanoate (0.086 g, 0.099 mmol) in DCM (1.8 mL) was added 4 N HCl in dioxane (0.25 mL). The reaction mixture was stirred at room temperature and was monitored by LCMS. At 16 hours, MTBE (20 ML) was added, and the reaction mixture was centrifuged (10,000×g for 15 min at 4° C.). The supernatant was drawn off, and the solids rinsed sparingly with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-aminobutyl)amino)-5-oxopentanoate dihydrochloride (0.066 g, 0.082 mmol, 82.8%) as a white solid. UPLC/ELSD: RT=2.22 min. MS (ES): m/z=670.59 (M+H)⁺ for C₄₂H₇₅N₃O₃. ¹H NMR (300 MHz, MeOD) δ 5.43-5.31 (m, 1H), 4.62-4.48 (m, 1H), 3.73-3.33 (m, 5H), 3.24-3.11 (m, 1H), 2.60-2.23 (m, 6H), 2.11-0.76 (m, 42H), 1.37 (d, J=6.6 Hz, 3H), 1.33 (d, J=6.6 Hz, 3H), 1.05 (s, 3H), 0.96 (d, J=6.4 Hz, 3H), 0.73 (s, 3H).

### CP. Compound SA157: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride

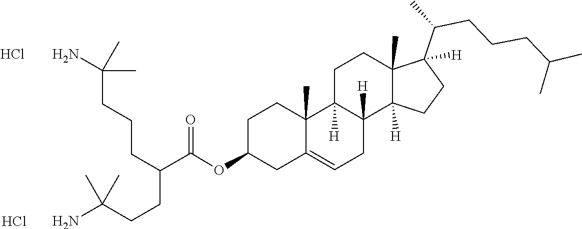

### Step 1: 4-((tert-butoxycarbonyl)amino)-4-methylpentyl 4-methylbenzenesulfonate

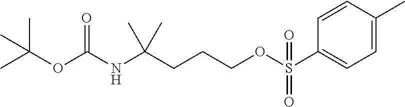 [SMILES: Cc1ccc(S(=O)(=O)OCCCC(C)(C)NC(=O)OC(C)(C)C)cc1]

To a solution of tert-butyl N-(5-hydroxy-2-methylpentan-2-yl)carbamate (2.50 g, 11.50 mmol) in dry DCM (30 mL) set stirring under nitrogen, was added triethylamine (8.02 mL, 57.52 mmol), dimethylaminopyridine (0.14 g, 1.15 mmol), and p-toluenesulfonyl chloride (4.39 g, 23.01 mmol). The solution was allowed to stir at room temperature for 6 hours, over which it turned a dark red color. The mixture was then further diluted with DCM, washed with water (1×30 mL), saturated aqueous sodium bicarbonate (1×30 mL) and brine (1×30 mL), dried over sodium sulfate, filtered, and concentrated to a dark brown oil. The oil was taken up in DCM and purified on silica in DCM with a 0-20% (80:19:1 DCM/MeOH/NH₄OH) gradient. Product-containing fractions were pooled and concentrated to give 4-((tert-butoxycarbonyl)amino)-4-methylpentyl 4-methylbenzenesulfonate as a light brown oil (3.55 g, 9.64 mmol, 83.0%). UPLC/ELSD: RT: 1.20 min. MS (ES): m/z (MH⁺) 372.4 for CisH₂₉NO₅S. ¹H NMR (300 MHz, CDCl₃) δ 7.78 (d, 2H, J=9 Hz), 7.36 (d, 2H, J=9 Hz), 4.37 (br. s, 1H), 4.00 (br. m, 2H), 2.45 (s, 3H), 1.64 (br. s, 4H), 1.40 (s, 10H), 1.20 (s, 6H).

### Step 2: tert-butyl (5-((3-((tert-butoxycarbonyl)amino)-3-methylbutyl)amino)-2-methylpentan-2-yl)carbamate

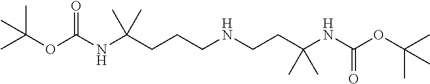 [SMILES: CC(C)(CCCNCCC(C)(C)NC(=O)OC(C)(C)C)NC(=O)OC(C)(C)C]

To a solution of tert-butyl N-[2-methyl-4-(2-nitrobenzenesulfonamido)butan-2-yl]carbamate (0.91 g, 2.34 mmol) in dry DMF (20 mL) stirring at room temperature under nitrogen, was added tert-butyl N-{2-methyl-5-[(4-methylbenzenesulfonyl)oxy]pentan-2-yl}carbamate (0.87 g, 2.34 mmol) and potassium carbonate (1.97 g, 14.25 mmol). The solution was warmed to 40° C. and stirred overnight. The following morning, the reaction was not complete by LC-MS, so it was heated to 100° C. and allowed to stir for an additional 3 hours. Then, the mixture was cooled to room temperature, and benzyl bromide (0.23 mL, 1.94 mmol) was added. The solution was stirred for 4 hours at room temperature and then thiophenol (0.92 mL, 8.99 mmol) was added, followed by additional potassium carbonate (0.97 g, 7.01 mmol) and DMF (20 mL). The solution stirred at room temperature overnight. The next morning, salts were removed from the mixture by centrifugation, and the supernatant was concentrated to a residue. The residue was taken up in 40 mL DCM, washed with water (2×10 mL) and brine (2×10 mL), dried over potassium carbonate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give tert-butyl (5-((3-((tert-butoxycarbonyl)amino)-3-methylbutyl)amino)-2-methylpentan-2-yl)carbamate as a colorless oil (0.49 g, 1.22 mmol, 52.24%). UPLC/ELSD: RT: 0.30 min. MS (ES): m/z (MH⁺) 402.4 for C₂₁H₄₃N₃O₄. ¹H NMR (300 MHz, CDCl₃) δ 5.88 (br. s, 1H), 4.69 (br. s, 1H), 2.61 (t, 2H), 2.52 (t, 2H), 1.61 (br. m, 4H), 1.35 (s, 21H), 1.18 (s, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate

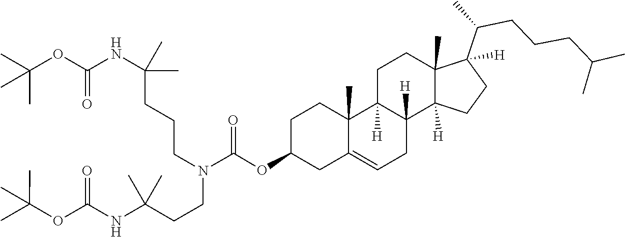

To a solution of tert-butyl (5-((3-((tert-butoxycarbonyl)amino)-3-methylbutyl)amino)-2-methylpentan-2-yl)carbamate (0.09 g, 0.22 mmol) in dry toluene (5 mL) set stirring under nitrogen was added triethylamine (0.07 mL, 0.09 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.12 g, 0.22 mmol) was added, and the solution was heated to 90° C. and allowed to proceed for 2 days. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate as a light yellow oil (0.16 g, 0.20 mmol, 89.9%). UPLC/ELSD: RT=3.67 min. MS (ES): m/z (MH⁺) 815.4 for C₄₉H₈₇N₃O₆. ¹H NMR (300 MHz, CDCl₃) δ 5.29 (br. s, 1H), 4.41 (br. m, 3H), 3.13 (br. m, 4H), 2.26 (br. m, 2H), 1.78 (br. m, 7H), 1.45 (br. m, 10H), 1.35 (s, 19H), 1.26 (br. m, 3H), 1.20 (s, 14H), 1.06 (br. m, 6H), 0.94 (s, 6H), 0.85 (d, 3H, J=6 Hz), 0.81 (d, 6H, J=6 Hz), 0.61 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride

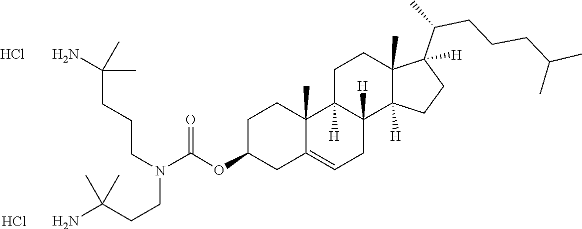

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate (0.16 g, 0.20 mmol) in DCM (5 mL) set stirring under nitrogen was added hydrochloric acid (4 M in dioxanes, 0.50 mL, 2.01 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride as a white solid (0.09 g, 0.12 mmol, 60.2%). UPLC/ELSD: RT=1.90 min. MS (ES): m/z (MH⁺) 614.3 for C₃₉H₇₃Cl₂N₃O₂. ¹H NMR (300 MHz, MeOD) δ 5.41 (br. s, 1H), 4.45 (br. m, 1H), 3.33 (br. m, 6H), 2.37 (br. m, 2H), 1.93 (br. m, 7H), 1.60 (br. m, 11H), 1.37 (s, 15H), 1.18 (br. m, 6H), 1.08 (s, 5H), 0.98 (d, 3H, J=6 Hz), 0.89 (d, 7H, J=6 Hz), 0.75 (s, 3H).

### CQ. Compound SA158: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-amino-3-methylbutyl)carbamate dihydrochloride

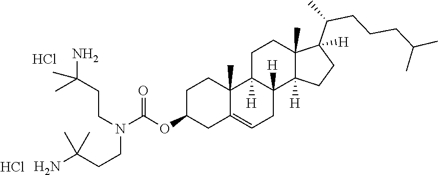

### Step 1: di-tert-butyl (azanediylbis(2-methylbutane-4,2-diyl))dicarbamate

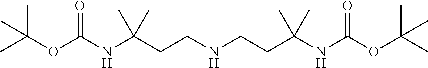 [SMILES: CC(C)(CCNCCC(C)(C)NC(=O)OC(C)(C)C)NC(=O)OC(C)(C)C]

Both tert-butyl (4-amino-2-methylbutan-2-yl)carbamate (0.50 g, 2.36 mmol) and tert-butyl (2-methyl-4-oxobutan-2-yl)carbamate (0.50 g, 2.36 mmol) were dissolve in 10 mL dry methanol and set stirring under nitrogen at room temperature. After two hours, sodium triacetoxyborohydride (1.25 g, 5.90 mmol) was added, and the reaction was allowed to continue stirring at room temperature overnight. The following morning, the amber reaction mixture was quenched with a few drops of water, concentrated to an orange oil, and taken back up in DCM. It was then washed with saturated sodium bicarbonate (1×15 mL) and brine (1×15 mL), dried over sodium sulfate, filtered, and concentrated to a yellow oil. The oil was resuspended in DCM and purified on silica in DCM with a 0-40% (50:45:5 DCM/MeOH/aqueous NH₄OH) gradient. Product-containing fractions were pooled and concentrated to give di-tert-butyl (azanediylbis(2-methylbutane-4,2-diyl))dicarbamate as a yellow oil (0.53 g, 1.37 mmol, 58.2%). UPLC/ELSD: RT=0.38 min. MS (ES): m/z (MH⁺) 388.6 for C₂₀H₄₁N₃O₄. ¹H NMR (300 MHz, CDCl₃) δ 5.56 (br. s, 2H), 2.67 (t, 4H), 1.77 (br. m, 5H), 1.62 (br. s, 4H), 1.44 (s, 18H), 1.30 (s, 12H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate

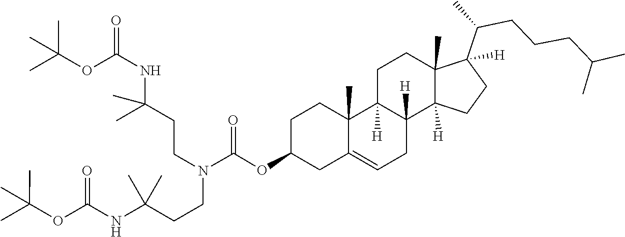

To a solution di-tert-butyl (azanediylbis(2-methylbutane-4,2-diyl))dicarbamate (0.10 g, 0.26 mmol) in dry toluene (5 mL) set stirring under nitrogen was added triethylamine (0.11 mL, 0.77 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.14 g, 0.26 mmol) was added, and the solution was heated to 90° C. and allowed to proceed for overnight. The following day, the reaction was not complete, so an additional 3 equivalents of triethylamine was added, and the reaction was allowed to proceed at 90° C. for an additional 24 hours. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate as a light yellow oil (0.15 g, 0.19 mmol, 72.2%). UPLC/ELSD: RT=3.41 min. MS (ES): mz (MHt) 801.4 for C₄₈H₈₅N₃O₆. ¹H NMR (300 MHz, CDCl₃) δ 5.29 (br. s, 1H), 4.57 (br. s, 1H), 4.44 (br. m, 2H), 3.15 (br. m, 4H), 2.26 (m, 2H), 1.80 (m, 9H), 1.42 (br. m, 7H), 1.35 (s, 19H), 1.27 (m, 3H), 1.21 (s, 13H), 1.05 (br. m, 8H), 0.95 (s, 6H), 0.85 (d, 4H, J=6 Hz), 0.80 (d, 6H, J=6 Hz), 0.60 (s, 3H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-amino-3-methylbutyl)carbamate dihydrochloride

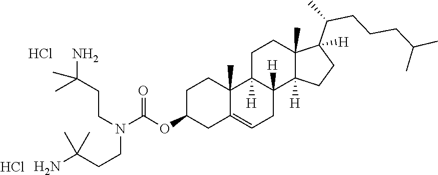

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate (0.15 g, 0.19 mmol) in DCM (3 mL) set stirring under nitrogen was added hydrochloric acid (4 M in dioxanes, 0.47 mL, 1.86 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-amino-3-methylbutyl)carbamate dihydrochloride as a white solid (0.10 g, 0.14 mmol, 74.1%). UPLC/ELSD: RT=1.78 min. MS (ES): m/z (MH⁺) 601.3 for C₃₈H₇₁C₂N₃O₂. ¹H NMR (300 MHz, MeOD) δ 5.42 (br. s, 1H), 4.45 (br. m, 1H), 3.40 (m, 6H), 2.41 (d, 2H, J=6 Hz), 1.94 (br. m, 10H), 1.56 (br. m, 7H), 1.41 (s, 16H), 1.17 (br. m, 7H), 1.06 (s, 6H), 0.98 (d, 4H, J=6 Hz), 0.90 (d, 6H, J=6 Hz), 0.75 (s, 4H).

### CR. Compound SA159: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-amino-3-methylbutyl)carbamate dihydrochloride

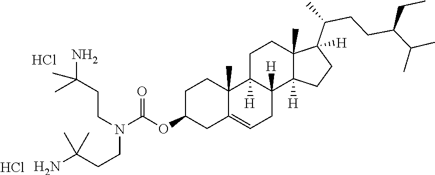

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate

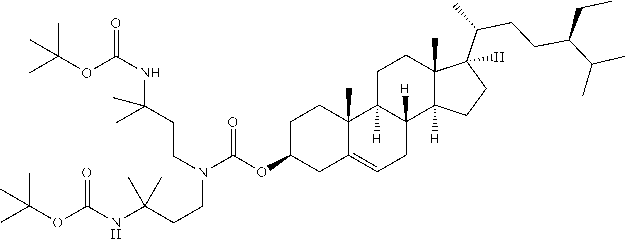

To a solution of di-tert-butyl (azanediylbis(2-methylbutane-4,2-diyl))dicarbamate (0.10 g, 0.26 mmol) in dry toluene (5 mL) set stirring under nitrogen was added triethylamine (0.11 mL, 0.77 mmol). Then, (1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,10H,11H-cyclopenta[a]phenanthren-7-yl 4-nitrophenyl carbonate (0.15 g, 0.26 mmol) was added, and the solution was heated to 90° C. and allowed to proceed for overnight. The following day, the reaction was not complete, so an additional 3 equivalents of triethylamine was added, and the reaction was allowed to proceed at 90° C. for an additional 24 hours. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate as a light yellow oil (0.17 g, 0.20 mmol, 78.6%). UPLC/ELSD: RT=3.55 min. MS (ES): m/z (MH⁺) 823.4 for C₅₀H₈₉N₃O₆. ¹H NMR (300 MHz, CDCl₃) δ 5.41 (br. s, 1H), 4.53 (br. m, 3H), 3.25 (br. m, 4H), 2.40 (m, 2H), 1.90 (br. m, 9H), 1.58 (m, 7H), 1.45 (s, 18H), 1.30 (s, 13H), 1.18 (br. m, 7H), 1.04 (s, 5H), 0.95 (d, 5H, J=6 Hz), 0.83 (q, 9H), 0.70 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-amino-3-methylbutyl)carbamate dihydrochloride

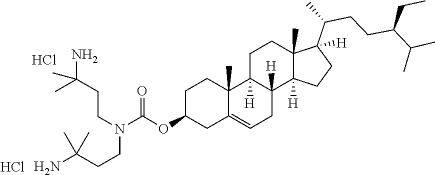

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate (0.17 g, 0.20 mmol) in DCM (3 mL) set stirring under nitrogen was added hydrochloric acid (4 M in dioxanes, 0.51 mL, 2.03 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-amino-3-methylbutyl)carbamate dihydrochloride as a white solid (0.12 g, 0.16 mmol, 77.4%). UPLC/ELSD: RT=1.94 min. MS (ES): m/z (MH⁺) 629.3 for C_{4D}H₇₅Cl₂N₃O₂. ¹H NMR (300 MHz, MeOD) δ 5.42 (br. s, 1H), 4.45 (br. m, 1H), 3.38 (br. m, 6H), 2.41 (d, 2H, J=6 Hz), 1.94 (br. m, 9H), 1.56 (br. m, 8H), 1.41 (s, 15H), 1.22 (br. m, 6H), 1.09 (s, 6H), 0.99 (d, 4H, J=6 Hz), 0.87 (q, 9H), 0.75 (s, 4H).

### CS. Compound SA160: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride

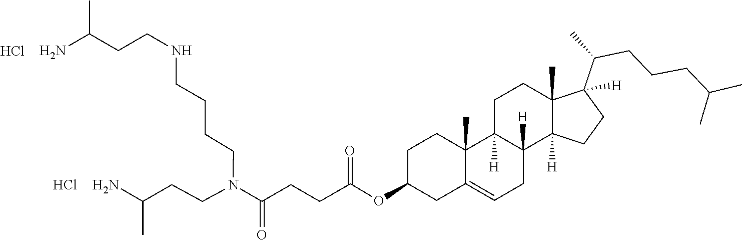

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate

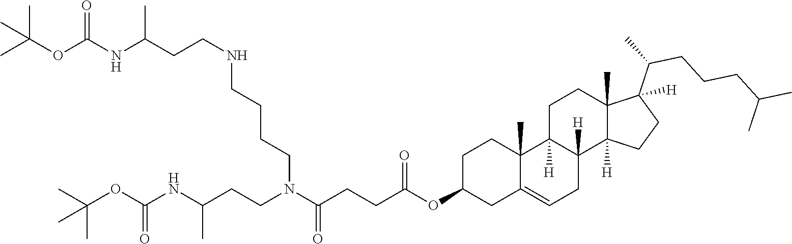

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.15 g, 0.31 mmol) in dry DCM (10 mL) stirring under nitrogen was added di-tert-butyl ((butane-1,4-diylbis(azanediyl))bis(butane-4,2-diyl))dicarbamate (0.33 g, 0.76 mmol), dimethylaminopyridine (0.08 g, 0.61 mmol), and 1-ethyl-3-(3-dimethylaminopropyl)carbodiimide hydrochloride (0.12 g, 0.61 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate as a light yellow oil (0.08 g, 0.09 mmol, 30.2%). UPLC/ELSD: RT: 2.49 min. MS (ES): m/z (MH⁺) 900.4 for C₅₃H₉₄N₄O₇. ¹H NMR (300 MHz, CDCl₃) δ 5.37 (br. s, 1H), 4.79 (br. m, 1H), 4.62 (br. m, 2H), 3.71 (br. m, 3H), 3.29 (m, 5H), 2.64 (br. m, 8H), 2.35 (t, 3H), 2.24 (s, 2H), 2.00 (br. m, 6H), 1.62 (br. m, 14H), 1.45 (s, 20H), 1.27 (br. m, 7H), 1.15 (m, 12H), 1.03 (s, 5H), 0.94 (d, 4H, J=6 Hz), 0.90 (d, 6H, J=6 Hz), 0.69 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride

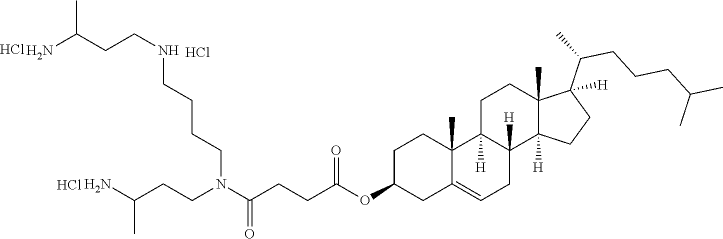

To a solution of (3S,8S,9S,1R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate (0.08 g, 0.09 mmol) in isopropanol (3 mL) set stirring under nitrogen was added hydrochloric acid (5.5 M in isopropanol, 0.19 mL, 0.92 mmol) dropwise. The solution was allowed to stir at room temperature overnight. The following morning, acetonitrile (25 mL) was added to the mixture, which was cooled to 0 15° C. and allowed to stir for 30 minutes. The solution was then filtered and washed with 3:1 acetonitrile/isopropanol. The resulting solid was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride as a white solid (0.05 g, 0.06 mmol, 60.0%). UPLC/ELSD: RT=1.36 min. MS (ES): m/z (MH⁺) 700.3 for C₄₃H₈₁Cl₃N₄O₃. ¹H NMR (300 MHz, MeOD) δ 5.28 (br. s, 1H), 4.41 (br. m, 1H), 3.85 (m, 1H), 3.39 (br. m, 5H), 3.20 (s, 2H), 3.02 (br. m, 5H), 2.60 (br. m, 4H), 2.23 (br. m, 4H), 1.92 (s, 5H), 1.71 (br. m, 9H), 1.43 (br. m, 8H), 1.28 (m, 12H), 1.06 (d, 11H, J=6 Hz), 0.95 (s, 6H), 0.86 (d, 4H, J=6 Hz), 0.80 (d, 6H, J=6 Hz), 0.63 (s, 3H).

### CT. Compound SA161: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride

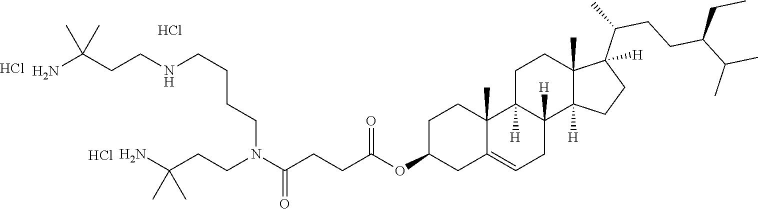

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate

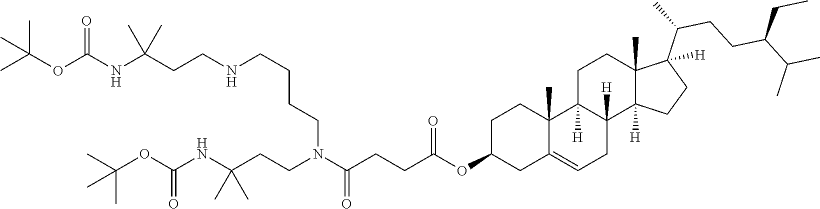

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.10 g, 0.19 mmol) in dry DCM (5 mL) stirring under nitrogen was added tert-butyl N-(4-{[4-({3-[(tert-butoxycarbonyl)amino]-3-methylbutyl}amino)butyl]amino}-2-methylbutan-2-yl)carbamate (0.22 g, 0.48 mmol), dimethylaminopyridine (0.05 g, 0.39 mmol), and 1-ethyl-3-(3-dimethylaminopropyl)carbodiimide hydrochloride (0.07 g, 0.39 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate as a light yellow oil (0.03 g, 0.03 mmol, 16.3%). UPLC/ELSD: RT: 2.77 min. MS (ES): m/z (MH⁺) 956.4 for C₅₇H₁₀₂N₄O₇. ¹H NMR (300 MHz, CDCl₃) δ 5.35 (br. s, 1H), 4.60 (br. m, 1H), 3.25 (br. m, 5H), 3.00 (br. m, 2H), 2.60 (br. m, 6H), 2.34 (d, 3H, J=6 Hz), 2.23 (m, 4H), 1.99 (br. m, 3H), 1.87 (br. m, 4H), 1.63 (br. m, 12H), 1.42 (s, 18H), 1.28 (d, 15H, J=6 Hz), 1.12 (br. m, 7H), 1.02 (s, 5H), 0.93 (d, 5H, J=6 Hz), 0.83 (q, 8H), 0.68 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride

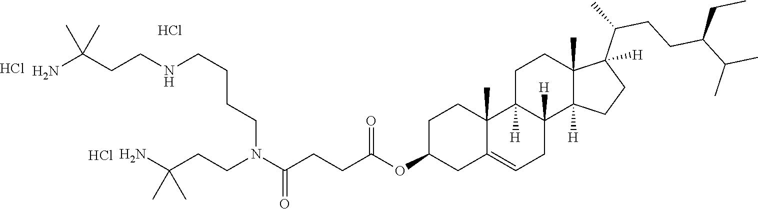

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate (0.03 g, 0.03 mmol) in DCM (1 mL) set stirring under nitrogen was added hydrochloric acid (4 M in dioxanes, 0.08 mL, 0.31 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride as a white solid (0.03 g, 0.03 mmol, 89.0%). UPLC/ELSD: RT=1.69 min. MS (ES): m/z (MH⁺) 756.3 for C₄₇H₈₉Cl₃N₄O₃. ¹H NMR (300 MHz, MeOD) δ 5.41 (br. s, 1H), 4.56 (br. m, 1H), 3.49 (br. m, 5H), 3.33 (br. s, 2H), 3.18 (br. m, 5H), 2.94 (m, 1H), 2.65 (br. m, 4H), 2.33 (d, 2H, J=6 Hz), 2.15 (br. m, 5H), 1.84 (br. m, 8H), 1.63 (br. m, 9H), 1.43 (t, 15H), 1.25 (br. m, 10H), 1.07 (s, 5H), 0.97 (d, 5H, J=6 Hz), 0.89 (q, 9H), 0.75 (s, 3H).

### CU. Compound SA162: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride

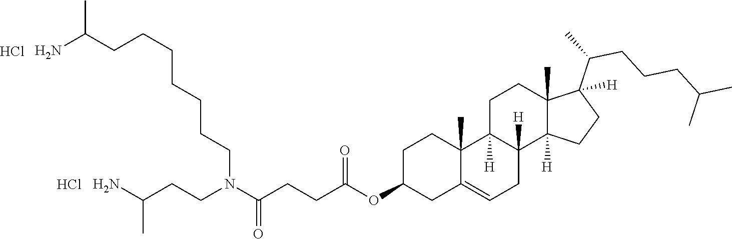

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate

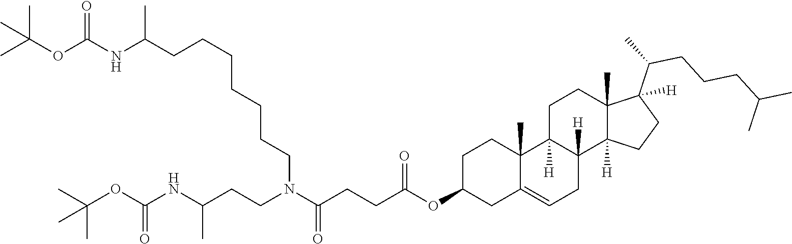

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.09 g, 0.18 mmol) in dry DCM (5 mL) stirring under nitrogen was added tert-butyl (9-((3-((tert-butoxycarbonyl)amino)butyl)amino)nonan-2-yl)carbamate (0.08 g, 0.18 mmol), dimethylaminopyridine (0.04 g, 0.35 mmol), and 1-ethyl-3-(3-dimethylaminopropyl)carbodiimide hydrochloride (0.07 g, 0.35 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate as a light yellow oil (0.11 g, 0.12 mmol, 67.6%). UPLC/ELSD: RT: 3.29 min. MS (ES): m/z (MH⁺) 899.4 for C₅₄H₉₅N₃O₇. ¹H NMR (300 MHz, CDCl₃) δ 5.30 (br. s, 1H), 4.53 (br. m, 2H), 4.34 (br. s, 1H), 3.54 (br. m, 2H), 3.14 (br. m, 4H), 2.56 (m, 4H), 2.26 (d, 2H, J=9 Hz), 1.85 (br. m, 5H), 1.49 (br. m, 9H), 1.36 (s, 19H), 1.24 (br. m, 13H), 1.02 (m, 13H), 0.94 (s, 5H), 0.85 (d, 4H, J=6 Hz), 0.81 (d, 6H, J=6 Hz), 0.60 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride

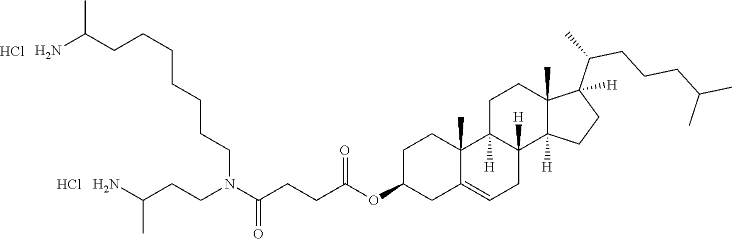

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate (0.11 g, 0.12 mmol) in DCM (3 mL) set stirring under nitrogen was added hydrochloric acid (4 N in dioxanes, 0.30 mL, 1.18 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride as a white solid (0.08 g, 0.10 mmol, 86.1%). UPLC/ELSD: RT=1.75 min. MS (ES): m/z (MH⁺) 699.3 for C₄₄H₈₁Cl₂N₃O₃. ¹H NMR (300 MHz, CDCl₃) δ 8.40 (m, 6H), 5.39 (br. s, 1H), 4.64 (br. m, 1H), 3.39 (br. m, 5H), 2.66 (s, 4H), 2.35 (d, 2H, J=6 Hz), 2.04 (br. m, 4H), 1.86 (m, 3H), 1.58 (br. m, 9H), 1.46 (br. m, 21H), 1.12 (br. m, 7H), 1.03 (s, 6H), 0.92 (d, 4H, J=6 Hz), 0.88 (d, 7H, J=6 Hz), 0.69 (s, 3H).

### CV. Compound SA163: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride

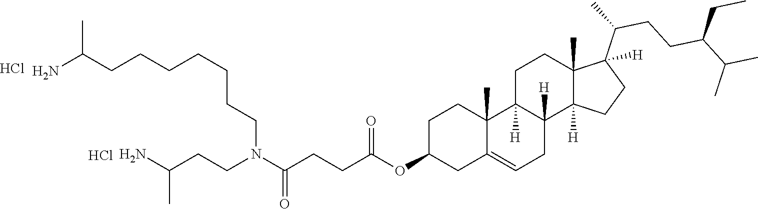

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate

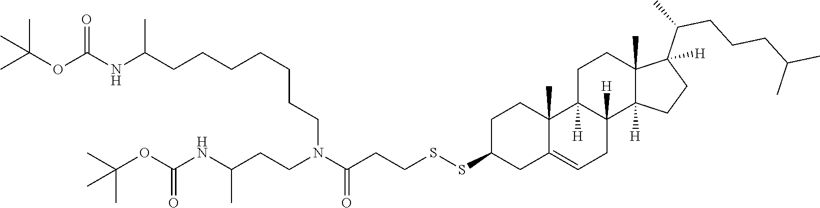

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.10 g, 0.19 mmol) in dry DCM (5 mL) stirring under nitrogen was added tert-butyl N-[4-({8-[(tert-butoxycarbonyl)amino]nonyl}amino)butan-2-yl]carbamate (0.08 g, 0.19 mmol), dimethylaminopyridine (0.05 g, 0.39 mmol), and 1-ethyl-3-(3-dimethylaminopropyl)carbodiimide hydrochloride (0.07 g, 0.39 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate as a light yellow oil (0.16 g, 0.17 mmol, 86.7%). UPLC/ELSD: RT: 3.65 min. MS (ES): m/z (MH⁺) 927.4 for C₅₆H₉₉N₃O₇. ¹H NMR (300 MHz, CDCl₃) δ 5.28 (br. s, 1H), 4.56 (br. m, 3H), 3.54 (br. m, 3H), 3.16 (br. m, 3H), 2.57 (m, 4H), 2.26 (d, 2H, J=3 Hz), 1.76 (br. m, 5H), 1.52 (br. m, 9H), 1.36 (s, 20H), 1.23 (br. m, 15H), 1.08 (br. m, 12H), 0.94 (s, 6H), 0.86 (d, 5H, J=6 Hz), 0.75 (q, 9H), 0.60 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride

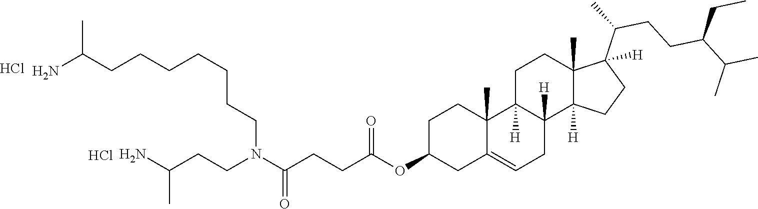

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate (0.16 g, 0.17 mmol) in DCM (3 mL) set stirring under nitrogen was added hydrochloric acid (4 M in dioxanes, 0.42 mL, 1.68 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride as a white solid (0.13 g, 0.13 mmol, 79.3%). UPLC/ELSD: RT=2.09 min. MS (ES): m/z (MH⁺) 728.3 for C₄₆H₈₅Cl₂N₃O₃. ¹H NMR (300 MHz, MeOD) δ 5.40 (br. s, 1H), 4.54 (br. m, 1H), 3.69 (br. m, 1H), 3.33 (s, 9H), 2.65 (br. m, 4H), 2.32 (d, 2H, J=6 Hz), 1.92 (br. m, 7H), 1.63 (br. m, 11H), 1.43 (br. m, 11H), 1.32 (t, 8H), 1.20 (br. m, 7H), 1.07 (s, 5H), 0.99 (d, 5H, J=6 Hz), 0.87 (q, 9H), 0.75 (s, 3H).

### CW. Compound SA164: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride

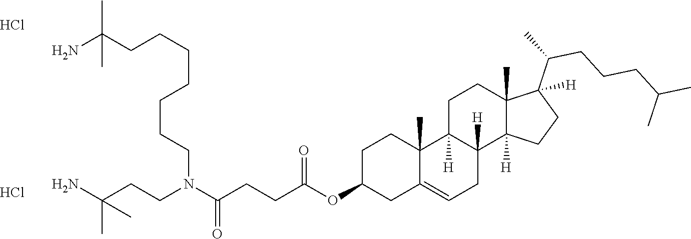

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate

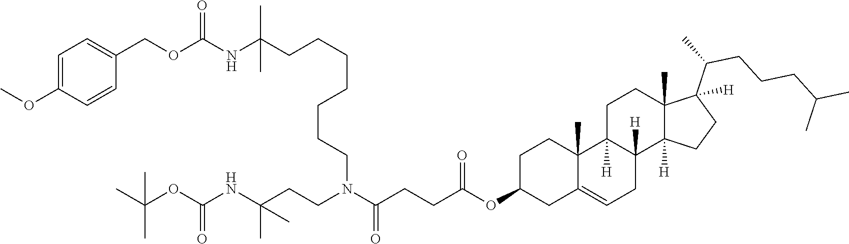

To a stirred solution of cholesteryl hemisuccinate (0.100 g, 0.205 mmol), tert-butyl N-(4-{[8-({[(4-methoxyphenyl)methoxy]carbonyl}amino)-8-methylnonyl]amino}-2-methylbutan-2-yl)carbamate (0.107 g, 0.205 mmol), and DMAP (cat.) in DCM (2 mL) cooled to 0° C. was added 1-ethyl-3-(3-dimethylaminopropyl)carbodiimide hydrochloride (0.079 g, 0.411 mmol). The reaction mixture was stirred at room temperature and was monitored by LCMS. At 16 hours, DMAP (0.050 g, 0.41 mmol) and 1-ethyl-3-(3-dimethylaminopropyl)carbodiimide hydrochloride (40 mg) were added. At 43 hours, 1-ethyl-3-(3-dimethylaminopropyl)carbodiimide hydrochloride (65 mg) was added. At 64 hours, the reaction mixture was diluted with DCM (15 mL), and then washed with 5% aq. NaHCO₃ soln. The aqueous was extracted with DCM (15 mL). The combined organics were washed with 5% aq. NaHCO₃ soln., passed through a hydrophobic frit, dried over Na₂SO₄, and concentrated. The crude material was purified via silica gel chromatography (0-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate (0.097 g, 0.098 mmol, 47.7%) as a clear oil. UPLC/ELSD: RT=3.68 min. MS (ES): m/z=990.87 (M+H)⁺ for C₆₀H₉₉N₃₀s. 1′H NMR (300 MHz, CDCl₃) δ 7.33-7.27 (m, 2H), 6.92-6.84 (m, 2H), 5.41-5.31 (m, 1H), 4.97 (s, 2H), 4.77-4.38 (m, 3H), 3.81 (s, 3H), 3.37-3.18 (m, 4H), 2.71-2.51 (m, 4H), 2.38-2.26 (m, 2H), 2.17-1.04 (m, 61H), 1.01 (s, 3H), 0.91 (d, J=5.9 Hz, 3H), 0.86 (d, J=6.6 Hz, 3H), 0.86 (d, J=6.6 Hz, 3H), 0.67 (s, 3H).

### Step 2: (3S,8S,9S,10R,13R,14S,17R)-10,13-dimethyl-17-((R)-6-methylheptan-2-yl)-2,3,4, 7, 8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride

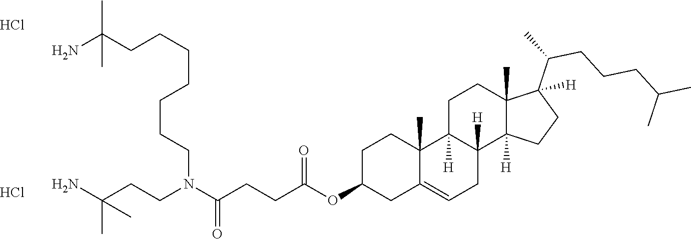

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate (0.093 g, 0.094 mmol) in DCM (1.5 mL) was added 4 N HCl in dioxane (0.17 mL). The reaction mixture was stirred at room temperature and was monitored by LCMS. At 17 hours, 4 N HCl in dioxane (0.07 mL) was added. At 22 hours, MTBE (10 mL) was added, and the reaction mixture was held at 4° C. overnight. The reaction mixture was blown down under N₂ stream until gelatinous. Then, ice cold MTBE (10 mL) was added, and the suspension was centrifuged (10,000×g for 1 h at 4° C.). The supernatant was decanted. The solids were rinsed with cold MTBE, suspended in MTBE, and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride (0.058 g, 0.068 mmol, 72.8%) as a white solid. UPLC/ELSD: RT=2.23 min. MS (ES): m/z=364.70 (M+2H)²⁺ for C₄₆H₈₃N₃O₃. ¹H NMR (300 MHz, MeOD) δ 5.43-5.34 (m, 1H), 4.62-4.45 (m, 1H), 3.56-3.34 (m, 4H), 2.73-2.57 (m, 4H), 2.42-2.26 (m, 2H), 2.14-1.77 (m, 7H), 1.75-0.97 (m, 33H), 1.05 (s, 3H), 1.36 (s, 6H), 1.33 (s, 6H), 0.95 (d, J=6.5 Hz, 3H), 0.88 (d, J=6.5 Hz, 6H), 0.73 (s, 3H).

### CX. Compound SA165: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride

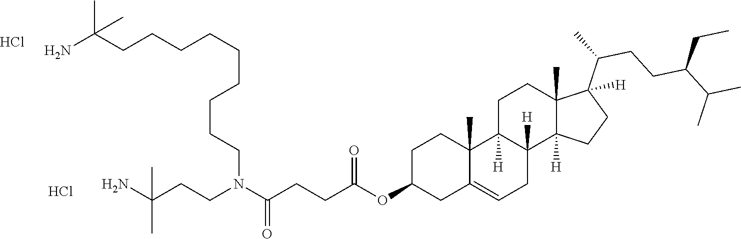

### Step 1: 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid

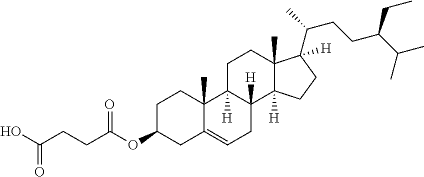

Sitosterol (3.00 g, 7.23 mmol) and succinic anhydride (0.941 g, 9.40 mmol) were combined in pyridine (6.0 mL). The reaction mixture was stirred at 80° C. and was monitored by TLC. At 19 hours, DMAP (cat.) added. At 89 hours, the reaction mixture was cooled to room temperature, diluted with DCM (100 mL), and washed with water. The organics were extracted with aq. 1 N NaOH (3×50 mL). A precipitate formed. The mixture was filtered. The solids were taken up in aq. 1 N HCl, and then extracted with DCM (3×50 mL). The organic extracts were washed with aq. 1 N HCl (2×) and water, passed through a hydrophobic frit, dried over Na₂SO₄, and concentrated. The residue was dissolved in DCM (5 mL) and hexanes (30 mL) was added while heating. Heat (hot water bath at 37° C.) was used to drive off solvent until solids formed. The solution was allowed to cool to room temperature, and was further cooled to 0° C. After 1.5 hours, white solids formed. The mixture was allowed to warm to room temperature, and solids were collected by vacuum filtration rinsing with cold 9:1 hexanes/DCM to afford 4-4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.436 g, 0.847 mmol, 11.7%) as an off-white solid. ¹H NMR (300 MHz, CDCl₃) δ 5.42-5.30 (m, 1H), 4.71-4.56 (m, 1H), 2.72-2.55 (m, 4H), 2.36-2.23 (m, 2H), 2.09-1.75 (m, 5H), 1.73-0.75 (m, 31H), 1.02 (s, 3H), 0.92 (d, J=6.4 Hz, 3H), 0.68 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate

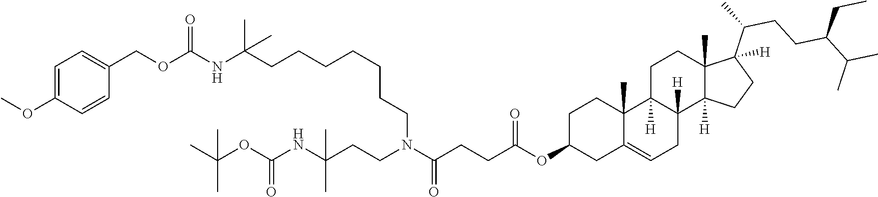

To a stirred solution of 4-4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.100 g, 0.194 mmol), tert-butyl N-(4-{[8-({[(4-methoxyphenyl)methoxy]carbonyl}amino)-8-methylnonyl]amino}-2-methylbutan-2-yl)carbamate (0.111 g, 0.214 mmol), and DMAP (cat.) in DCM (2.0 mL) cooled to 0° C. was added 1-ethyl-3-(3-dimethylaminopropyl)carbodiimide hydrochloride (0.074 g, 0.39 mmol). The reaction was mixture stirred at room temperature and was monitored by LCMS. At 16 hours, DMAP (0.047 g, 0.39 mmol) was added, followed by 1-ethyl-3-(3-dimethylaminopropyl)carbodiimide hydrochloride (45 mg). At 43 hours, 1-ethyl-3-(3-dimethylaminopropyl)carbodiimide hydrochloride (65 mg) was added. At 64 hours, the reaction mixture was diluted with DCM (15 mL), and washed with 5% aq. NaHCO₃ soln. The aqueous was extracted with DCM (15 mL). The combined organics were washed with 5% aq. NaHCO₃ soln., passed through a hydrophobic frit, dried over Na₂SO₄, and concentrated. The crude material was purified via silica gel chromatography (0-50% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate (0.097 g, 0.095 mmol, 49.0%) as a clear oil. UPLC/ELSD: RT=3.74 min. MS (ES): m/z=1018.87 (M+H)⁺ for C₆₂H₁₀₃N₃O₈. ¹H NMR (300 MHz, CDCl₃) δ 7.34-7.26 (m, 2H), 6.92-6.83 (m, 2H), 5.39-5.32 (m, 1H), 4.97 (s, 2H), 4.80-4.36 (m, 3H), 3.80 (s, 3H), 3.39-3.18 (m, 4H), 2.69-2.52 (m, 4H), 2.35-2.23 (m, 2H), 2.12-0.77 (m, 71H), 1.01 (s, 3H), 0.92 (d, J=6.4 Hz, 3H), 0.67 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride

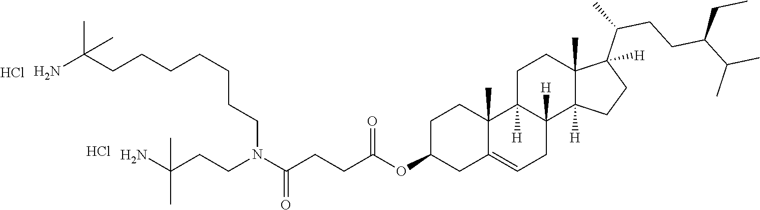

To a stirred solution (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl4-((3-((tert-butoxycarbonyl)amino)-3-methylbutyl)(8-((((4-methoxybenzyl)oxy)carbonyl)amino)-8-methylnonyl)amino)-4-oxobutanoate (0.093 g, 0.091 mmol) in DCM (1.5 mL) was added 4 N HCl in dioxane (0.17 mL). The reaction mixture was stirred at room temperature and was monitored by LCMS. At 17 hours, 4 N HCl in dioxane (0.07 mL) was added. At 22 hours, MTBE (10 mL) added, and the reaction mixture was held at 4° C. overnight. The reaction mixture was blown down under a stream of N₂ until gelatinous. Cold MTBE (10 mL) was added, and the suspension was centrifuged (10,000×g for 1 h at 4° C.). The supernatant was decanted, the solids were rinsed with cold MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride (0.034 g, 0.039 mmol, 42.2%) as a white solid. UPLC/ELSD: RT=2.34 min. MS (ES): m/z=377.76 (M+2H)²⁺ for C₄₈H₈₇N₃O₃. ¹H NMR (300 MHz, MeOD) δ 5.46-5.32 (m, 1H), 4.65-4.43 (m, 1H), 3.54-3.34 (m, 4H), 2.74-2.50 (m, 4H), 2.45-2.21 (m, 2H), 2.13-1.79 (m, 7H), 1.77-0.78 (m, 43H), 1.36 (s, 6H), 1.32 (s, 6H), 1.05 (s, 3H), 0.96 (d, J=6.5 Hz, 3H), 0.73 (s, 3H).

### CY. Compound SA166: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride

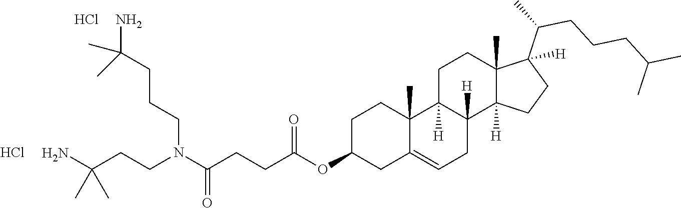

### Step 1: (3S,8S,9S,10R,13R,14S,17R)-10,13-dimethyl-17-((R)-6-methylheptan-2-yl)-2,3,4, 7, 8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate

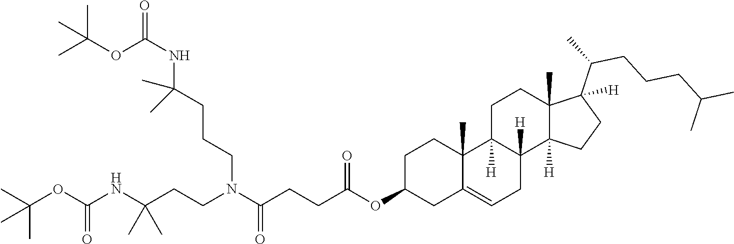

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.11 g, 0.22 mmol) in dry DCM (5 mL) stirring under nitrogen was added tert-butyl (5-((3-((tert-butoxycarbonyl)amino)-3-methylbutyl)amino)-2-methylpentan-2-yl)carbamate (0.09 g, 0.22 mmol), dimethylaminopyridine (0.06 g, 0.45 mmol), and 1-ethyl-3-(3-dimethylaminopropyl)carbodiimide hydrochloride (0.09 g, 0.45 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate as a light yellow oil (0.17 g, 0.19 mmol, 85.6%). UPLC/ELSD: RT: 3.55 min. MS (ES): m/z (MH⁺) 871.4 for C₅₂H₉₁N₃O₇. ¹H NMR (300 MHz, CDCl₃) δ 5.37 (br. s, 1H), 4.62 (br. m, 3H), 3.27 (br. m, 4H), 2.64 (br. m, 4H), 2.35 (d, 2H, J=6 Hz), 2.01 (br. m, 3H), 1.85 (br. m, 4H), 1.56 (br. m, 11H), 1.44 (s, 17H), 1.35 (br. m, 3H), 1.30 (m, 13H), 1.14 (br. m, 6H), 1.03 (s, 5H), 0.95 (d, 3H, J=6 Hz), 0.90 (d, 6H, J=6 Hz), 0.70 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride

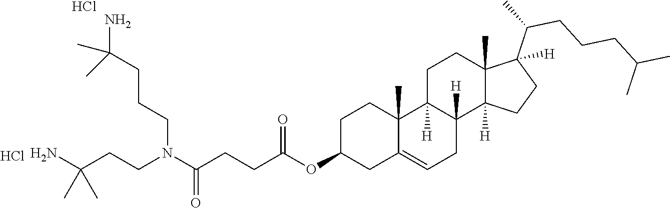

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate (0.17 g, 0.19 mmol) in DCM (5 mL) set stirring under nitrogen was added hydrochloric acid (4 M in dioxanes, 0.48 mL, 1.92 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride as a white solid (0.12 g, 0.14 mmol, 74.0%). UPLC/ELSD: RT=1.99 min. MS (ES): m/z (MH⁺) 671.3 for C₄₂H₇₇Cl₂N₃O₃. ¹H NMR (300 MHz, MeOD) δ 5.52 (s, 1H), 5.39 (br. s, 1H), 4.56 (br. m, 1H), 3.68 (s, 1H), 3.47 (br. m, 3H), 3.32 (br. s, 2H), 2.64 (br. m, 4H), 2.35 (br. m, 2H), 1.90 (br. m, 6H), 1.55 (br. m, 10H), 1.46 (s, 3H), 1.40 (br. s, 14H), 1.16 (br. m, 5H), 1.07 (s, 5H), 0.98 (d, 5H, J=6 Hz), 0.89 (d, 8H, J=6 Hz), 0.75 (s, 3H).

### CZ. Compound SA167: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride

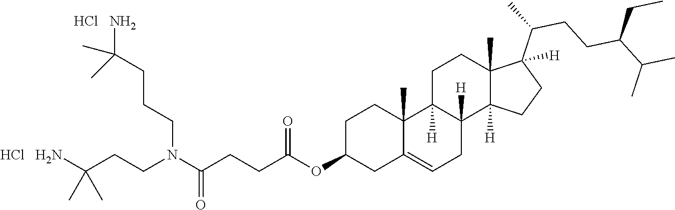

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate

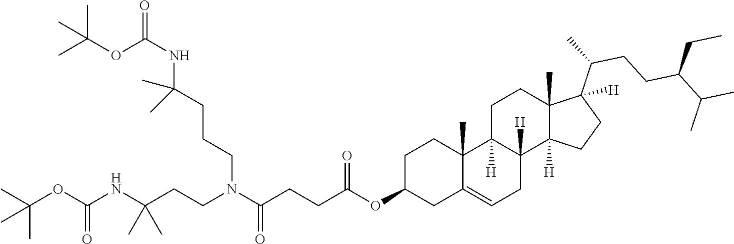

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.12 g, 0.22 mmol) in dry DCM (5 mL) stirring under nitrogen was added tert-butyl (5-((3-((tert-butoxycarbonyl)amino)-3-methylbutyl)amino)-2-methylpentan-2-yl)carbamate (0.09 g, 0.22 mmol), dimethylaminopyridine (0.06 g, 0.45 mmol), and 1-ethyl-3-(3-dimethylaminopropyl)carbodiimide hydrochloride (0.09 g, 0.45 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate as a light yellow oil (0.14 g, 0.15 mmol, 67.6%). UPLC/ELSD: RT: 3.64 min. MS (ES): m/z (MH⁺) 899.4 for C₅₄H₉₅N₃O₇. ¹H NMR (300 MHz, CDCl₃) δ 5.31 (br. s, 1H), 4.57 (br. m, 3H), 3.24 (br. m, 4H), 2.57 (br. m, 4H), 2.30 (d, 2H, J=6 Hz), 2.00 (br. m, 4H), 1.82 (br. m, 4H), 1.53 (br. m, 11H), 1.38 (s, 18H), 1.31 (br. m, 2H), 1.24 (m, 16H), 1.11 (br. m, 6H), 0.98 (s, 6H), 0.88 (d, 5H, J=6 Hz), 0.79 (q, 10H), 0.64 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride

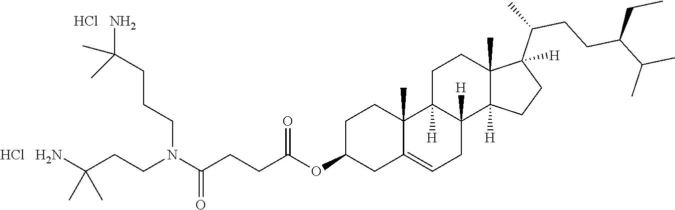

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate (0.14 g, 0.15 mmol) in DCM (5 mL) set stirring under nitrogen was added hydrochloric acid (4 M in dioxanes, 0.38 mL, 1.51 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride as a white solid (0.10 g, 0.12 mmol, 80.3%). UPLC/ELSD: RT=2.18 min. MS (ES): m/z (MH⁺) 699.3 for C₄₄H₈₁Cl₂N₃O₃. ¹H NMR (300 MHz, MeOD) δ 5.41 (br. s, 1H), 4.53 (br. m, 1H), 3.46 (br. m, 3H), 3.32 (m, 5H), 2.65 (br. m, 4H), 2.33 (br. m, 2H), 1.91 (br. m, 7H), 1.64 (br. m, 11H), 1.45 (s, 3H), 1.40 (s, 13H), 1.20 (br. m, 7H), 1.07 (s, 5H), 0.99 (d, 4H, J=6 Hz), 0.87 (q, 9H), 0.75 (s, 4H).

### DA. Compound SA168: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-aminobutyl)amino)-4-oxobutanoate dihydrochloride

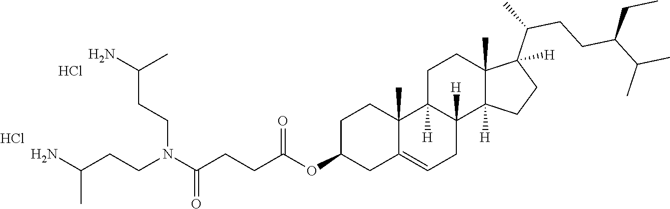

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)butyl)amino)-4-oxobutanoate

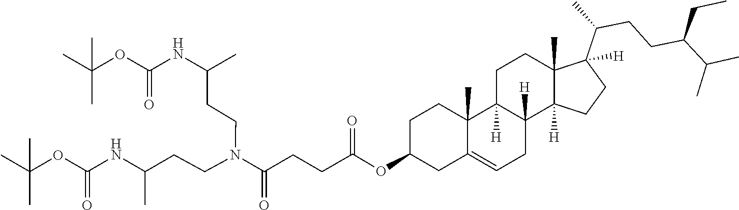

To a stirred solution of 4-4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.100 g, 0.194 mmol), tert-butyl N-[4-({3-[(tert-butoxycarbonyl)amino]butyl}amino)butan-2-yl]carbamate (0.077 g, 0.21 mmol), and DMAP (0.052 g, 0.43 mmol) in DCM (2.0 mL) was added 1-ethyl-3-(3-dimethylaminopropyl)carbodiimide hydrochloride (0.074 g, 0.39 mmol). The reaction mixture was stirred at room temperature and was monitored by LCMS. At 17 hours, the reaction mixture was diluted with DCM (15 mL), then washed with 5% aq. NaHCO₃ soln. The aqueous was extracted with DCM (15 mL). The combined organics were passed through a hydrophobic frit, dried over Na₂SO₄, and concentrated. The crude material was purified via silica gel chromatography (20-65% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)butyl)amino)-4-oxobutanoate (0.129 g, 0.151 mmol, 77.6%) as a clear oil. UPLC/ELSD: RT=3.53 min. MS (ES): m/z=856.81 (M+H)⁺ for C₅₁H₈₉N₃O₇. ¹H NMR (300 MHz, CDCl₃) δ 5.41-5.32 (m, 1H), 4.70-4.35 (m, 3H), 3.78-3.08 (m, 6H), 2.74-2.46 (m, 4H), 2.38-2.23 (m, 2H), 2.10-0.75 (m, 64H), 1.01 (s, 3H), 0.92 (d, J=6.3 Hz, 3H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-aminobutyl)amino)-4-oxobutanoate dihydrochloride

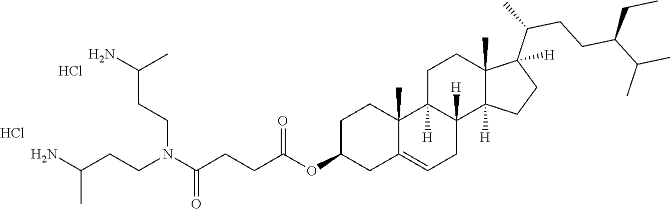

To a stirred solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)butyl)amino)-4-oxobutanoate (0.125 g, 0.146 mmol) in DCM (2.5 mL) was added 4 N HCl in dioxane (0.37 mL). The reaction mixture was stirred at room temperature and was monitored by LCMS. At 16 hours, MTBE (20 mL) was added, and the reaction mixture was centrifuged (10,000×g for 15 min at 4° C.). The supernatant was drawn off, and the solids rinsed sparingly with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-aminobutyl)amino)-4-oxobutanoate dihydrochloride (0.084 g, 0.11 mmol, 74.8%) as a white solid. UPLC/ELSD: RT=2.11 min. MS (ES): m/z=349.41 [(M+2H)+CH₃CN]²⁺ for C₄₁H₇₃N₃O₃. ¹H NMR (300 MHz, MeOD) δ 5.43-5.34 (m, 1H), 4.62-4.46 (m, 1H), 3.75-3.34 (m, 5H), 3.26-3.15 (m, 1H), 2.83-2.59 (m, 4H), 2.42-2.25 (m, 2H), 2.16-0.78 (m, 40H), 1.38 (d, J=6.6 Hz, 3H), 1.32 (d, J=6.5 Hz, 3H), 1.05 (s, 3H), 0.96 (d, J=6.4 Hz, 3H), 0.73 (s, 3H).

### DB. Compound SA169: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride

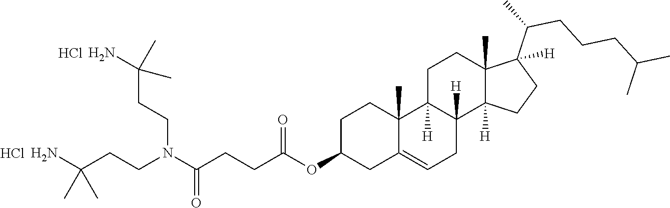

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate

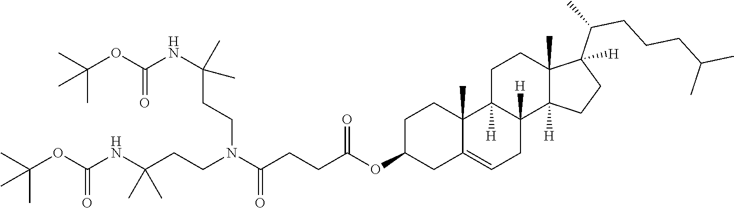

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.13 g, 0.26 mmol) in dry DCM (5 mL) stirring under nitrogen was added di-tert-butyl (azanediylbis(2-methylbutane-4,2-diyl))dicarbamate (0.10 g, 0.26 mmol), dimethylaminopyridine (0.06 g, 0.52 mmol), and 1-ethyl-3-(3-dimethylaminopropyl)carbodiimide hydrochloride (0.10 g, 0.52 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate as a light yellow oil (0.22 g, 0.26 mmol, 100.0%). UPLC/ELSD: RT: 3.58 min. MS (ES): m/z (MH⁺) 857.4 for C₅₁H₈₉N₃O₇. ¹H NMR (300 MHz, CDCl₃) δ 5.07 (br. s, 1H), 4.55 (br. s, 1H), 4.32 (br. m, 1H), 4.20 (br. s, 1H), 3.04 (br. m, 4H), 2.33 (s, 4H), 2.05 (br. s, 2H), 1.65 (br. m, 9H), 1.28 (br. m, 7H), 1.14 (s, 19H), 1.00 (s, 15H), 0.83 (br. m, 7H), 0.74 (s, 6H), 0.65 (d, 4H, J=6 Hz), 0.60 (d, 6H, J=6 Hz), 0.39 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride

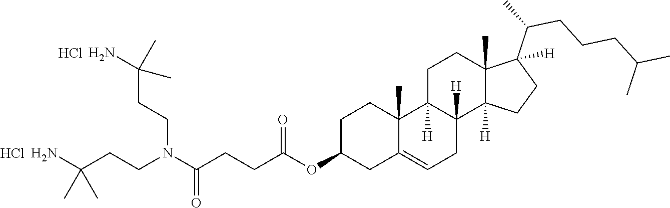

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate (0.22 g, 0.26 mmol) in DCM (5 mL) set stirring under nitrogen was added hydrochloric acid (4 M in dioxanes, 0.65 mL, 2.59 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride as a white solid (0.12 g, 0.16 mmol, 62.4%). UPLC/ELSD: RT=2.03 min. MS (ES): m/z (MH⁺) 657.3 for C₄₁H₇₅Cl₂N₃O₃. ¹H NMR (300 MHz, MeOD) δ 5.39 (br. s, 1H), 4.53 (br. m, 1H), 3.48 (br. m, 4H), 3.33 (br. s, 3H), 2.67 (br. m, 4H), 2.33 (br. m, 2H), 2.04 (br. m, 3H), 1.91 (br. m, 6H), 1.55 (br. m, 7H), 1.46 (s, 6H), 1.40 (s, 8H), 1.16 (br. m, 11H), 1.07 (s, 6H), 0.96 (d, 4H, J=6 Hz), 0.89 (d, 8H, J=6 Hz), 0.75 (s, 4H).

### DC. Compound SA170: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride

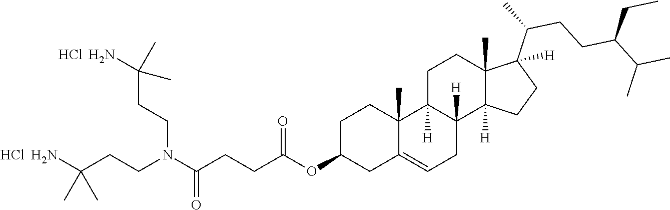

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate

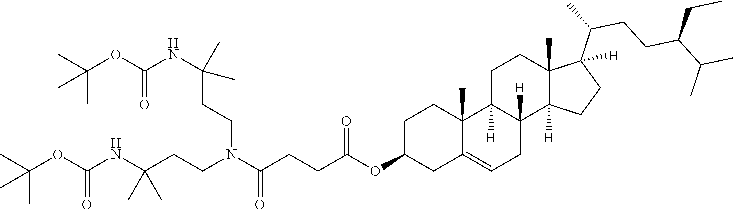

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.13 g, 0.26 mmol) in dry DCM (5 mL) stirring under nitrogen was added di-tert-butyl (azanediylbis(2-methylbutane-4,2-diyl))dicarbamate (0.10 g, 0.26 mmol), dimethylaminopyridine (0.06 g, 0.52 mmol), and 1-ethyl-3-(3-dimethylaminopropyl)carbodiimide hydrochloride (0.10 g, 0.52 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate as a light yellow oil (0.18 g, 0.21 mmol, 80.2%). UPLC/ELSD: RT: 3.65 min. MS (ES): m/z (MH⁺) 885.4 for C₅₃H₉₃N₃O₇. 11H NMR (300 MHz, CDCl₃) δ 5.06 (br. s, 1H), 4.54 (br. s, 1H), 4.33 (br. m, 1H), 4.21 (s, 1H), 3.03 (m, 4H), 2.32 (s, 4H), 2.02 (d, 2H, J=6 Hz), 1.63 (br. m, 9H), 1.29 (br. m, 7H), 1.14 (s, 19H), 0.99 (s, 15H), 0.84 (br. m, 6H), 0.73 (s, 5H), 0.65 (d, 5H, J=6 Hz), 0.54 (q, 9H), 0.39 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride

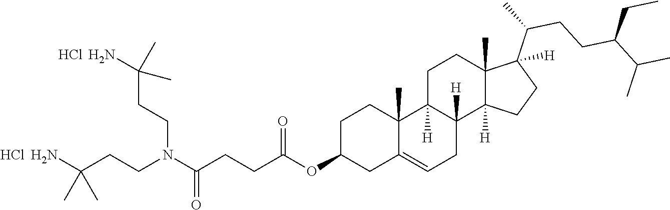

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate (0.18 g, 0.21 mmol) in DCM (5 mL) set stirring under nitrogen was added hydrochloric acid (4 M in dioxanes, 0.52 mL, 2.07 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride as a white solid (0.16 g, 0.19 mmol, 91.5%). UPLC/ELSD: RT=2.13 min. MS (ES): m/z (MH⁺) 685.3 for C₄₃H₇₉C₂N₃O₃. ¹H NMR (300 MHz, MeOD) δ 5.39 (br. s, 1H), 4.53 (br. m, 1H), 3.53 (m, 4H), 3.33 (br. s, 3H), 2.67 (d, 4H, J=3 Hz), 2.33 (d, 2H, J=6 Hz), 2.04 (br. m, 3H), 1.93 (br. m, 6H), 1.58 (br. m, 8H), 1.46 (s, 7H), 1.40 (s, 8H), 1.25 (br. m, 11H), 1.07 (s, 5H), 0.99 (m, 5H), 0.87 (q, 10H), 0.75 (s, 3H).

### DD. Compound SA171: N-(3-aminobutyl)-N-(4-((3-aminobutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

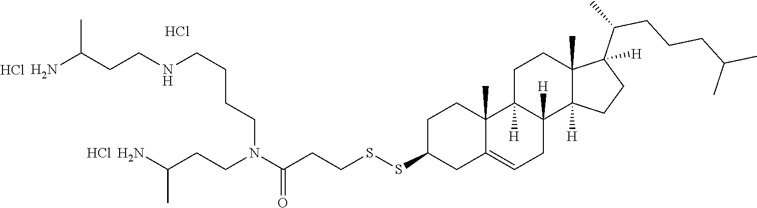

### Step 1: tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6-trimethyl-4-oxo-3-oxa-5,9,14-triazaoctadecan-17-yl)carbamate

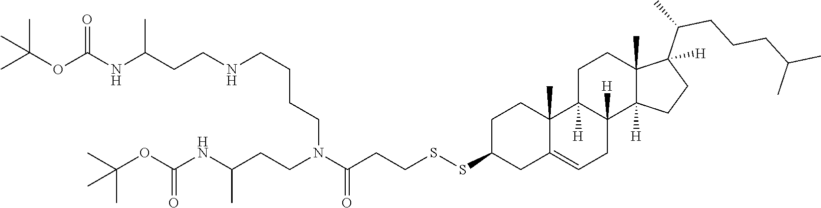

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.15 g, 0.30 mmol) in dry DCM (10 mL) stirring under nitrogen was added tert-butyl N-(4-{[4-({3-[(tert-butoxycarbonyl)amino]butyl}amino)butyl]amino}butan-2-yl)carbamate (0.32 g, 0.74 mmol), dimethylaminopyridine (0.07 g, 0.59 mmol), and 1-ethyl-3-(3-dimethylaminopropyl)carbodiimide hydrochloride (0.11 g, 0.59 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6-trimethyl-4-oxo-3-oxa-5,9,14-triazaoctadecan-17-yl)carbamate as a light yellow oil (0.08 g, 0.09 mmol, 28.7%). UPLC/ELSD: RT: 2.79 min. MS (ES): m/z (MH⁺) 920.4 for Cs₂H₉₄N₄O₅S₂. ¹H NMR (300 MHz, CDCl₃) δ 5.37 (br. s, 1H), 4.79 (br. m, 2H), 3.64 (br. m, 3H), 3.31 (br. m, 4H), 2.95 (t, 2H), 2.69 (br. m, 7H), 2.34 (d, 2H, J=6 Hz), 2.24 (m, 1H), 1.94 (br. m, 4H), 1.60 (br. m, 11H), 1.44 (s, 22H), 1.31 (br. m, 5H), 1.16 (br. m, 13H), 1.00 (s, 6H), 0.93 (d, 4H, J=6 Hz), 0.88 (d, 4H, J=6 Hz), 0.68 (s, 3H).

### Step 2: N-(3-aminobutyl)-N-(4-((3-aminobutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-JH-cyclopenta[a]phenanthren-3-yl)disulfaneyl)propanamide trihydrochloride

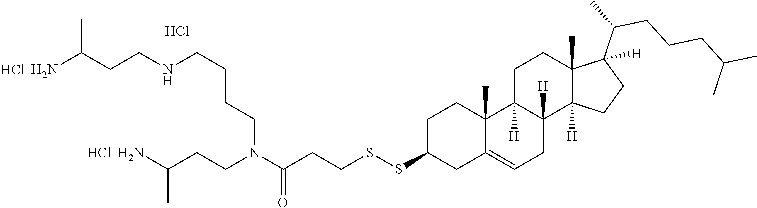

To a solution of tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6-trimethyl-4-oxo-3-oxa-5,9,14-triazaoctadecan-17-yl)carbamate (0.08 g, 0.09 mmol) in DCM (2 mL) set stirring under nitrogen was added hydrochloric acid (4 M in dioxanes, 0.21 mL, 0.85 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give N-(3-aminobutyl)-N-(4-((3-aminobutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride as a white solid (0.05 g, 0.05 mmol, 63.1%). UPLC/ELSD: RT=1.77 min. MS (ES): m/z (MH⁺) 720.3 for C₄₂H₈₁Cl₃N₄OS₂. ¹H NMR (300 MHz, MeOD) δ 5.39 (br. s, 1H), 3.68 (br. m, 1H), 3.46 (br. m, 6H), 3.33 (s, 4H), 3.14 (br. m, 6H), 3.00 (br. m, 5H), 2.63 (br. m, 1H), 2.37 (d, 2H, J=6 Hz), 1.99 (br. m, 16H), 1.53 (br. m, 8H), 1.40 (m, 13H), 1.18 (br. m, 8H), 1.05 (s, 4H), 0.98 (d, 5H, J=6 Hz), 0.90 (d, 8H, J=6 Hz), 0.74 (s, 3H).

### DE. Compound SA172: N-(3-aminobutyl)-N-(8-aminononyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

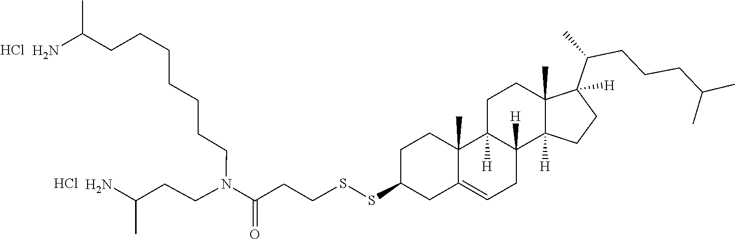

### Step 1: tert-butyl (4-(N-(8-((tert-butoxycarbonyl)amino)nonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)butan-2-yl)carbamate

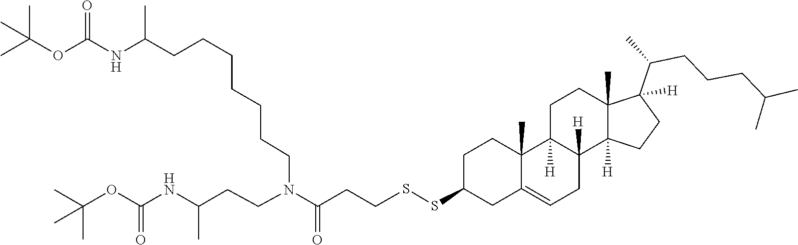

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.09 g, 0.18 mmol) in dry DCM (5 mL) stirring under nitrogen was added tert-butyl (9-((3-((tert-butoxycarbonyl)amino)butyl)amino)nonan-2-yl)carbamate (0.08 g, 0.18 mmol), dimethylaminopyridine (0.04 g, 0.35 mmol), and 1-ethyl-3-(3-dimethylaminopropyl)carbodiimide hydrochloride (0.07 g, 0.35 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give tert-butyl (4-(N-(8-((tert-butoxycarbonyl)amino)nonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)butan-2-yl)carbamate as a light yellow oil (0.11 g, 0.12 mmol, 66.1%). UPLC/ELSD: RT: 3.50 min. MS (ES): m/z (MH⁺) 919.4 for C₅₃H₉₅N₃O₅S2. ¹H NMR (300 MHz, CDCl₃) δ 5.29 (br. s, 1H), 4.59 (br. s, 1H), 4.35 (br. s, 1H), 3.55 (br. m, 3H), 3.17 (br. m, 3H), 2.89 (t, 2H), 2.63 (br. m, 3H), 2.27 (br. m, 2H), 1.86 (m, 5H), 1.51 (br. m, 9H), 1.36 (s, 19H), 1.24 (br. m, 14H), 1.05 (m, 13H), 0.93 (s, 6H), 0.86 (d, 4H, J=6 Hz), 0.80 (d, 6H, J=6 Hz), 0.61 (s, 3H).

### Step 2: N-(3-aminobutyl)-N-(8-aminononyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

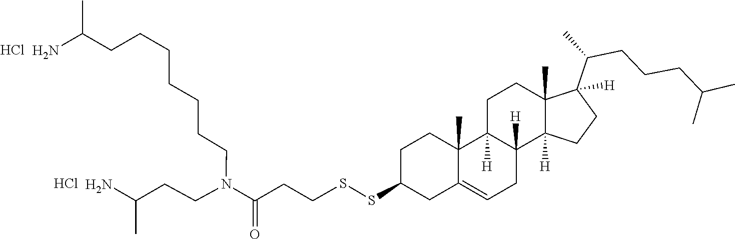

To a solution of tert-butyl (4-(N-(8-((tert-butoxycarbonyl)amino)nonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)butan-2-yl)carbamate (0.11 g, 0.12 mmol) in DCM (3 mL) set stirring under nitrogen was added hydrochloric acid (4 N in dioxanes, 0.29 mL, 1.15 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give N-(3-aminobutyl)-N-(8-aminononyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride as a white solid (0.09 g, 0.11 mmol, 94.3%). UPLC/ELSD: RT=1.86 min. MS (ES): m/z (MH⁺) 719.3 for C₄₃H₈₁Cl₂N₃OS₂. ¹H NMR (300 MHz, CDCl₃) δ 8.39 (br. m, 6H), 5.38 (br. s, 1H), 3.41 (br. m, 5H), 3.00 (br. s, 2H), 2.85 (br. s, 2H), 2.70 (br. m, 1H), 2.35 (br. m, 2H), 2.01 (m, 8H), 1.44 (br. m, 28H), 1.12 (br. m, 7H), 1.02 (s, 6H), 0.94 (d, 3H, J=6 Hz), 0.87 (d, 7H, J=6 Hz), 0.69 (s, 3H).

### DF. Compound SA173: N,N-bis(3-aminobutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

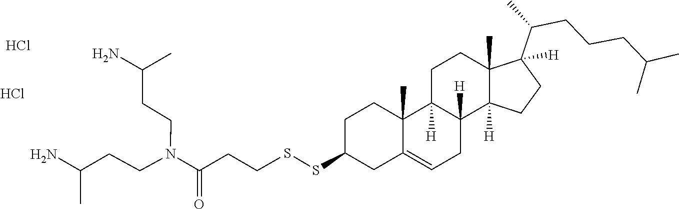

### Step 1: di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(butane-4,2-diyl))dicarbamate

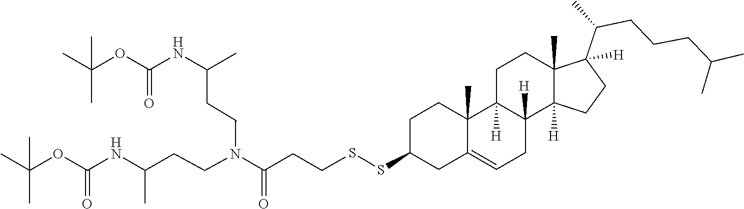

To a stirred solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.100 g, 0.197 mmol), tert-butyl N-[4-({3-[(tert-butoxycarbonyl)amino]butyl}amino)butan-2-yl]carbamate (0.078 g, 0.22 mmol), and triethylamine (0.08 mL, 0.6 mmol) in DCM (1.6 mL) cooled to 0° C. was added 50 wt % propanephosphonic acid anhydride in DCM (0.20 mL, 0.39 mmol) dropwise. The reaction mixture was stirred at room temperature and was monitored by LCMS. At 17 hours, the reaction mixture was diluted with DCM (10 mL), then washed with 5% aq. NaHCO₃ soln. The aqueous was extracted with DCM (10 mL). The combined organics were passed through a hydrophobic frit, dried over Na₂SO₄, and concentrated. The crude material was purified via silica gel chromatography (0-50% EtOAc in hexanes) to afford di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(butane-4,2-diyl))dicarbamate (0.124 g, 0.146 mmol, 74.1%) as a clear oil. UPLC/ELSD: RT=3.60 min. MS (ES): m/z=849.65 (M+H)⁺ for C₄₈H₈₅N₃O₅S2. ¹H NMR (300 MHz, CDCl3) δ 5.39-5.32 (m, 1H), 4.68-4.38 (m, 2H), 3.77-3.12 (m, 6H), 3.06-2.85 (m, 2H), 2.80-2.54 (m, 3H), 2.41-2.23 (m, 2H), 2.03-0.94 (m, 54H), 1.00 (s, 3H), 0.91 (d, J=6.4 Hz, 3H), 0.87 (d, J=6.6 Hz, 3H), 0.86 (d, J=6.6 Hz, 3H), 0.67 (s, 3H).

### Step 2: N,N-bis(3-aminobutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

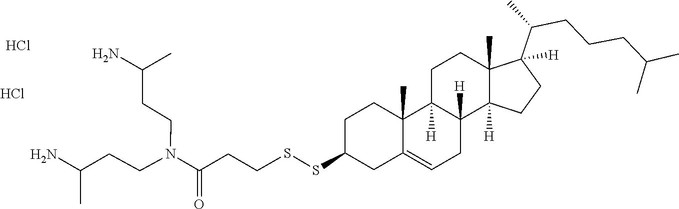

To a stirred solution of di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(butane-4,2-diyl))dicarbamate (0.121 g, 0.143 mmol) in DCM (2.5 mL) cooled to 0° C. was added 4 N HCl in dioxane (0.36 mL) dropwise. The reaction mixture was stirred at room temperature and was monitored by LCMS. At 16 hours, MTBE (20 mL) was added, and the reaction mixture was centrifuged (10,000×g for 15 min at 4° C.). The supernatant was removed, and the solids rinsed sparingly with MTBE. Solids were suspended in MTBE, then concentrated to afford N,N-bis(3-aminobutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride (0.089 g, 0.12 mmol, 83.6%) as a white solid. UPLC/ELSD: RT=2.12 min. MS (ES): m/z=648.64 (M+H)⁺ for C₃₈H₆₉N₃OS₂. ¹H NMR (300 MHz, MeOD) δ 5.45-5.31 (m, 1H), 3.77-3.33 (m, 5H), 3.27-3.17 (m, 1H), 3.05-2.74 (m, 4H), 2.73-2.59 (m, 1H), 2.42-2.27 (m, 2H), 2.15-1.75 (m, 9H), 1.72-0.96 (m, 21H), 1.39 (d, J=6.8 Hz, 3H), 1.33 (d, J=6.5 Hz, 3H), 1.03 (s, 3H), 0.95 (d, J=6.4 Hz, 3H), 0.88 (d, J=6.6 Hz, 6H), 0.73 (s, 3H).

### DG. Compound SA174: N,N-bis(3-amino-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

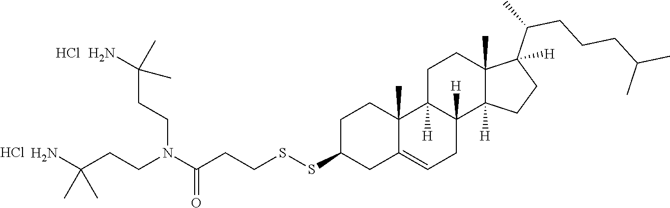

### Step 1: di-tert-butyl (((3-(((3S,8S,9S,10R,13R,14S,17R)-10,13-dimethyl-17-((R)-6-methylheptan-2-yl)-2,3,4, 7, 8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate

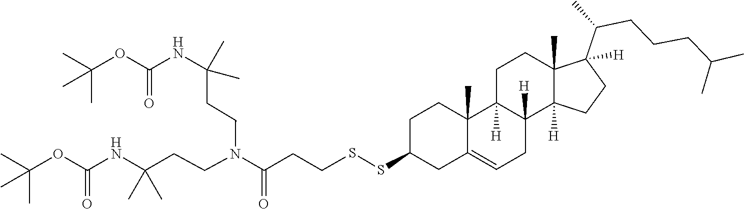

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.13 g, 0.26 mmol) in dry DCM (5 mL) stirring under nitrogen was added di-tert-butyl (azanediylbis(2-methylbutane-4,2-diyl))dicarbamate (0.10 g, 0.26 mmol), dimethylaminopyridine (0.06 g, 0.52 mmol), and 1-ethyl-3-(3-dimethylaminopropyl)carbodiimide hydrochloride (0.10 g, 0.52 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate as a light yellow oil (0.21 g, 0.24 mmol, 92.9%). UPLC/ELSD: RT: 3.72 min. MS (ES): m/z (MH⁺) 877.4 for C₅₀H₈₉N₃O₅S₂. ¹H NMR (300 MHz, CDCl₃) δ 5.07 (br. s, 1H), 5.02 (s, 1H), 4.50 (s, 1H), 4.18 (s, 1H), 3.01 (br. m, 4H), 2.64 (t, 2H), 2.43 (br. m, 3H), 2.03 (br. m, 2H), 1.61 (br. m, 9H), 1.27 (br. m, 5H), 1.14 (s, 19H), 0.99 (s, 15H), 0.83 (br. m, 8H), 0.71 (s, 5H), 0.64 (d, 4H, J=6 Hz), 0.58 (d, 6H, J=6 Hz), 0.39 (s, 3H).

### Step 2: N,N-bis(3-amino-3-methylbutyl)-3-(((3S,8S,9S,10R,13R,14S,17R)-10,13-dimethyl-17-((R)-6-methylheptan-2-yl)-2,3,4,7, 8,9,10,11,12,13,14,15,16,17-tetradecahydro-1H-cyclopenta[a]phenanthren-3-yl)disulfaneyl)propanamide dihydrochloride

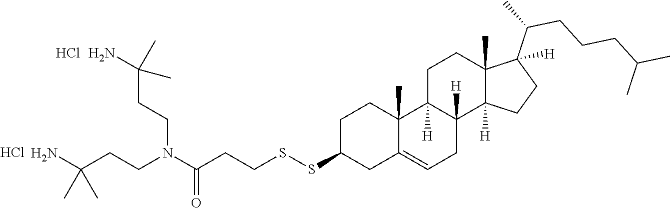

To a solution of di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate (0.21 g, 0.24 mmol) in DCM (5 mL) set stirring under nitrogen was added hydrochloric acid (4 M in dioxanes, 0.60 mL, 2.40 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give N,N-bis(3-amino-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride as a white solid (0.14 g, 0.17 mmol, 71.9%). UPLC/ELSD: RT=2.17 min. MS (ES): m/z (MH⁺) 677.3 for C₄₀H₇₅Cl₂N₃OS₂. ¹H NMR (300 MHz, MeOD) δ 5.40 (br. s, 1H), 3.51 (br. m, 4H), 3.32 (br. s, 2H), 2.99 (t, 2H), 2.86 (t, 2H), 2.64 (br. m, 1H), 2.37 (d, 2H, J=6 Hz), 2.07 (br. m, 9H), 1.55 (br. m, 8H), 1.47 (s, 6H), 1.41 (s, 8H), 1.18 (br. m, 11H), 1.05 (s, 5H), 0.96 (d, 4H, J=6 Hz), 0.92 (d, 7H, J=6 Hz), 0.74 (s, 3H).

### DH. Compound SA175: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride

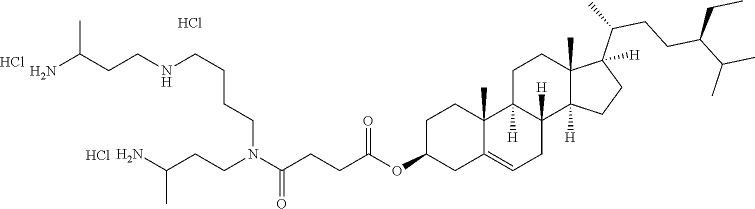

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate

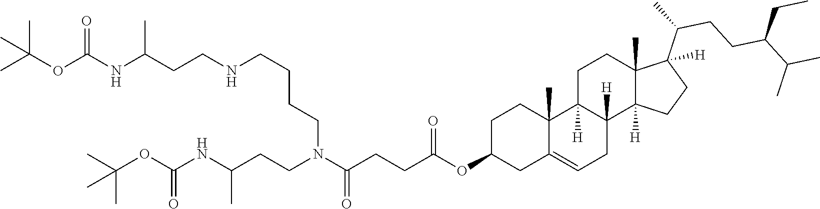

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.10 g, 0.19 mmol) in dry DCM (5 mL) stirring under nitrogen was added tert-butyl N-(4-{[4-({3-[(tert-butoxycarbonyl)amino]butyl}amino)butyl]amino}butan-2-yl)carbamate (0.21 g, 0.48 mmol), dimethylaminopyridine (0.05 g, 0.39 mmol), and 1-ethyl-3-(3-dimethylaminopropyl)carbodiimide hydrochloride (0.07 g, 0.39 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate as a light yellow oil (0.03 g, 0.03 mmol, 14.6%). UPLC/ELSD: RT: 2.76 min. MS (ES): m/z (MH⁺) 928.4 for C₅₅H₉₈N₄O₇. ¹H NMR (300 MHz, CDCl₃) δ 5.30 (br. s, 1H), 4.70 (br. m, 1H), 4.53 (br. m, 2H), 3.57 (br. m, 2H), 3.25 (br. m, 4H), 2.57 (br. m, 8H), 2.26 (d, 3H, J=6 Hz), 1.77 (br. m, 6H), 1.54 (br. m, 13H), 1.37 (s, 20H), 1.17 (br. m, 5H), 1.08 (br. m, 12H), 0.94 (s, 5H), 0.86 (d, 5H, J=6 Hz), 0.78 (q, 9H), 0.61 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride

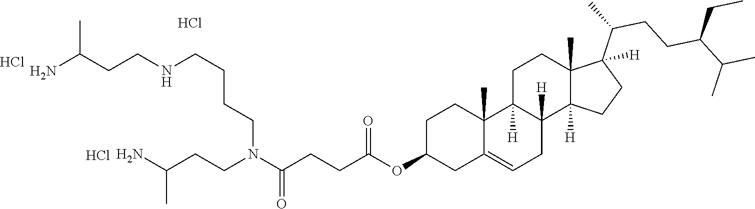

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate (0.03 g, 0.03 mmol) in DCM (1 mL) set stirring under nitrogen was added hydrochloric acid (4 M in dioxanes, 0.07 mL, 0.28 mmol) dropwise. The solution was allowed to stir at room temperature overnight. The following morning, hexanes (5 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride as a white solid (0.02 g, 0.02 mmol, 63.9%). UPLC/ELSD: RT=1.75 min. MS (ES): m/z (MH⁺) 728.3 for C₄₅H₈₅C₃N₄O₃. ¹H NMR (300 MHz, MeOD) δ 5.40 (br. s, 1H), 4.55 (br. m, 1H), 3.68 (br. s, 1H), 3.50 (br. m, 4H), 3.33 (br. m, 3H), 3.14 (br. m, 5H), 2.66 (br. m, 4H), 2.33 (br. m, 3H), 1.81 (br. m, 19H), 1.37 (m, 11H), 1.20 (br. m, 6H), 1.07 (s, 5H), 0.99 (d, 5H, J=6 Hz), 0.89 (q, 9H), 0.75 (s, 3H).

### DI. Compound SA176: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride

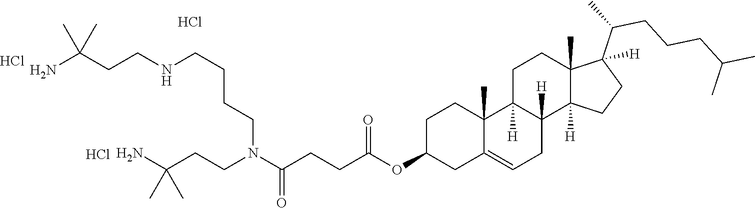

### Step 1: (3S,8S,9S,10R,13R,14S,17R)-10,13-dimethyl-17-((R)-6-methylheptan-2-yl)-2,3,4, 7, 8,9,10,11,12,13,14,15,16,17-tetradecahydro-JH-cyclopenta[a]phenanthren-3-yl 14-(3-((tert-butoxycarbonyl)amino)-3-methylbutyl)-2,2,6,6-tetramethyl-4,1S-dioxo-3-oxa-S,9,14-triazaoctadecan-18-oate

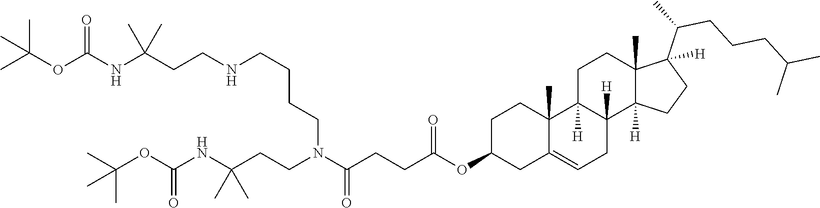

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.15 g, 0.31 mmol) in dry DCM (10 mL) stirring under nitrogen was added tert-butyl N-(4-{[4-({3-[(tert-butoxycarbonyl)amino]-3-methylbutyl}amino)butyl]amino}-2-methylbutan-2-yl)carbamate (0.35 g, 0.76 mmol), dimethylaminopyridine (0.8 g, 0.61 mmol), and 1-ethyl-3-(3-dimethylaminopropyl)carbodiimide hydrochloride (0.12 g, 0.61 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate as a light yellow oil (0.10 g, 0.10 mmol, 33.6%). UPLC/ELSD: RT: 2.70 min. MS (ES): m/z (MH⁺) 928.4 for C₅₅H₉₈N₄O₇. ¹H NMR (301 MHz, CDCl₃) δ 5.70 (br. s, 1H), 5.29 (br. s, 1H), 4.50 (br. m, 2H), 3.21 (br. m, 4H), 2.54 (br. m 8H), 2.24 (d, 2H, J=6 Hz), 1.87 (br. m, 7H), 1.53 (br. m, 10H), 1.36 (s, 22H), 1.23 (s, 15H), 1.02 (br. m, 7H), 0.94 (s, 6H), 0.85 (d, 4H, J=6 Hz), 0.80 (d, 6H, J=6 Hz), 0.60 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride

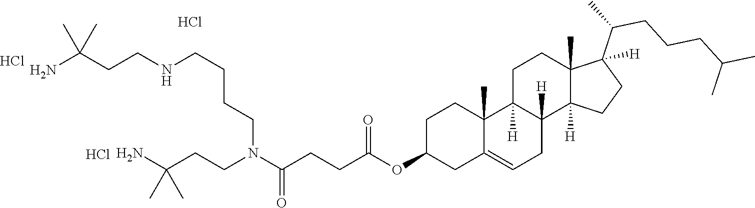

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate (0.10 g, 0.10 mmol) in DCM (2 mL) set stirring under nitrogen was added hydrochloric acid (4 M in dioxanes, 0.26 mL, 1.02 mmol) dropwise. The solution was allowed to stir at room temperature overnight. The following morning, hexanes (5 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride as a white solid (0.09 g, 0.08 mmol, 74.4%). UPLC/ELSD: RT=1.72 min. MS (ES): m/z (MH⁺) 728.3 for C₄₅H₈₅Cl₃N₄O₃. ¹H NMR (300 MHz, MeOD) δ 5.40 (br. s, 1H), 4.54 (br. m, 1H), 3.69 (br. s, 1H), 3.49 (br. m, 4H), 3.32 (br. s, 6H), 3.17 (br. m, 5H), 2.66 (br. m, 4H), 2.33 (br. m, 2H), 2.05 (br. m, 10H), 1.66 (br. m, 16H), 1.43 (br. m, 16H), 1.32 (br. s, 16H), 1.16 (br. m, 8H), 1.07 (s, 5H), 0.92 (br. m, 25H), 0.75 (s, 3H).

### DJ. Compound SA177: N,N-bis(3-amino-3-methylbutyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

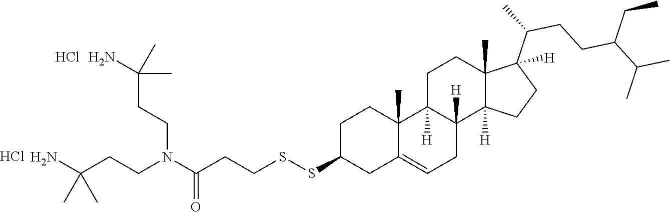

### Step 1: Sitosteryl Chloride

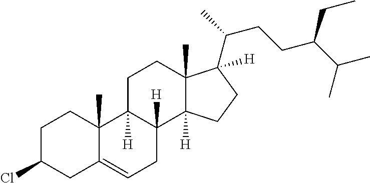

Sitosterol (3.000 g, 7.234 mmol) and thionyl chloride (3.00 mL, 41.4 mmol) were combined in PhMe (30 mL). The reaction mixture was stirred at 80° C. and was monitored by TLC. At 20 hours, the reaction mixture was concentrated, then re-concentrated from PhMe (2×). The solids were dissolved in hot 3:1 EtOH/EtOAc (45 mL), then allowed to cool to room temperature. Solid precipitated out of solution. The mother liquor was decanted, and the solids were rinsed sparingly with cold 3:1 EtOH/EtOAc to afford sitosteryl chloride (2.534 g, 5.850 mmol, 80.9%) as a clear solid. UPLC/ELSD: RT=3.74 min. ¹H NMR (300 MHz, CDCl₃) δ 5.41-5.33 (m, 1H), 3.87-3.67 (m, 1H), 2.69-2.39 (m, 2H), 2.17-1.75 (m, 6H), 1.73-0.76 (m, 30H), 1.03 (s, 3H), 0.92 (d, J=6.5 Hz, 3H), 0.68 (s, 3H). ¹³C NMR (75 MHz, CDCl₃) δ 140.96, 122.64, 60.50, 56.85, 56.19, 50.22, 45.98, 43.56, 42.46, 39.85, 39.27, 36.53, 36.29, 34.09, 33.53, 31.98, 31.93, 29.30, 28.38, 26.22, 24.43, 23.22, 21.11, 19.98, 19.41, 19.19, 18.93, 12.13, 12.00.

### Step 2: Sitosteryl Thiocyanate

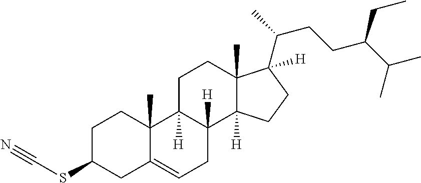

Sitosteryl chloride (2.748 g, 6.344 mmol) and sodium thiocyanate (19.235 g, 237.27 mmol) were refluxed in EtOH (105 mL). The reaction was monitored by TLC. At 64 hours, the reaction mixture was filtered hot, rinsing with a copious amount of DCM. The filtrate was concentrated, taken up in DCM (150 mL), and washed with water. The organic layer was passed through a hydrophobic frit, dried over Na₂SO₄, and concentrated. The resultant solid was dissolved in near boiling 1:1 EtOAc/hexanes (19 mL), then allowed to cool slowly. Once reaching room temperature, the mixture was further cooled to 4° C. Solids were collected by vacuum filtration, rinsing sparingly with cold 1:1 EtOAc/hexanes to afford sitosteryl thiocyanate (2.183 g, 4.789 mmol, 75.5%) as an off-white solid. UPLC/ELSD: RT=3.47 min. ¹H NMR (300 MHz, CDCl₃) δ 5.48-5.35 (m, 1H), 3.23-2.97 (m, 1H), 2.65-2.35 (m, 2H), 2.10-0.76 (m, 36H), 1.03 (s, 3H), 0.92 (d, J=6.4 Hz, 3H), 0.68 (s, 3H). ¹³C NMR (75 MHz, CDCl3) δ 140.10, 123.29, 111.40, 56.80, 56.18, 50.20, 48.23, 45.98, 42.45, 39.89, 39.79, 39.51, 36.61, 36.28, 34.08, 31.96, 31.86, 30.10, 29.29, 28.37, 26.21, 24.41, 23.21, 21.06, 19.97, 19.35, 19.18, 18.93, 12.13, 12.00.

### Step 3: Thiositosterol

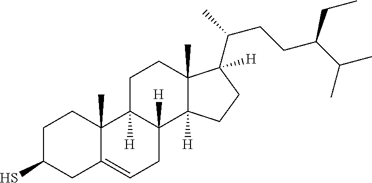

To a stirred solution of THF (30 mL) and 2.3 M lithium aluminum hydride in 2-methyltetrahydrofuran (4.7 mL) was added dropwise a solution of sitosteryl thiocyanate (2.100 g, 4.607 mmol) in PhMe (20 mL) dropwise over 15 min. The reaction mixture was stirred at room temperature and was monitored by TLC. At 2.5 hours, the reaction mixture was cooled to 0° C., then aq. 3 N HCl (50 mL) was added slowly dropwise over 10 min. Upon completion of addition, the layers were separated. The aqueous layer was extracted with MTBE (3×30 mL). The combined organics layers were washed with water and brine, dried over Na₂SO₄, and concentrated to afford thiositosterol (1.944 g, 4.513 mmol, 97.9%) as a white solid. UPLC/ELSD: RT=3.70 min. ¹H NMR (300 MHz, CDCl₃) δ 5.43-5.22 (m, 1H), 2.80-2.60 (m, 1H), 2.45-2.23 (m, 2H), 2.10-1.75 (m, 5H), 1.74-0.78 (m, 32H), 1.00 (s, 3H), 0.92 (d, J=6.4 Hz, 3H), 0.67 (s, 3H). ¹³C NMR (75 MHz, CDCl₃) δ 142.07, 121.19, 56.90, 56.20, 50.35, 45.98, 44.35, 42.45, 40.08, 39.89, 39.60, 36.49, 36.30, 34.22, 34.09, 31.95, 29.29, 28.39, 26.21, 24.43, 23.21, 21.04, 19.98, 19.48, 19.18, 18.93, 12.13, 12.00.

### Step 4: 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)pyridine

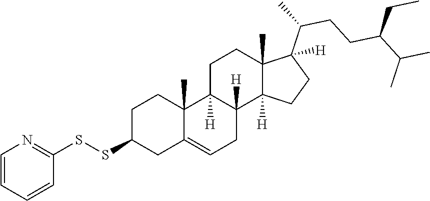

Thiositosterol (1.92 g, 4.46 mmol) and Aldrithiol (1.08 g, 4.90 mmol) were combined in chloroform (12 mL). The reaction mixture was stirred at room temperature and was monitored by LCMS. At 24 hours, Aldrithiol (0.25 g) was added. At 6 days, the reaction mixture was concentrated, taken up in MeOH (30 mL), and sonicated. The solids were collected by vacuum filtration rinsing sparingly with MeOH to afford 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)pyridine (2.098 g, 3.886 mmol, 87.2%) as a light yellow solid. UPLC/ELSD: RT=3.56 min. MS (ES): m/z=540.62 (M+H)⁺ for C₃₄H₅₃NS₂. ¹H NMR (300 MHz, CDCl₃) δ 8.51-8.39 (m, 1H), 7.83-7.71 (m, 1H), 7.69-7.57 (m, 1H), 7.13-7.00 (m, 1H), 5.40-5.28 (m, 1H), 2.89-2.69 (m, 1H), 2.41-2.28 (m, 2H), 2.08-1.75 (m, 5H), 1.74-0.74 (m, 31H), 0.98 (s, 3H), 0.91 (d, J=6.3 Hz, 3H), 0.67 (s, 3H).

### Step 5: 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate

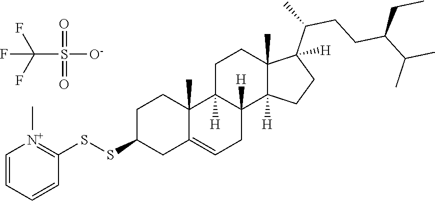

To a solution of 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)pyridine (2.000 g, 3.704 mmol) in heptanes (30 mL) and DCM (3.0 mL) was added methyl trifluoromethanesulfonate (0.51 mL, 4.5 mmol) dropwise over 10 min. The reaction mixture was stirred at room temperature and was monitored by TLC. At 19 hours, solids were collected by vacuum filtration rinsing with heptanes to afford 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (2.443 g, 3.470 mmol, 93.7%) as a white solid. UPLC/ELSD: RT=2.67 min. MS (ES): m/z=554.80 (M)j for C₃₅H₅₆NS₂. ¹H NMR (300 MHz, CD₃CN) δ 8.60-8.49 (m, 2H), 8.39-8.30 (m, 1H), 7.75-7.66 (m, 1H), 5.40-5.35 (m, 1H), 4.19 (s, 3H), 3.04-2.89 (m, 1H), 2.49-2.32 (m, 2H), 2.08-0.76 (m, 36H), 1.01 (s, 3H), 0.93 (d, J=6.5 Hz, 3H), 0.69 (s, 3H).

### Step 6: 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid

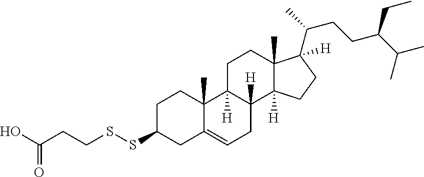

To a solution of 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (2.400 g, 3.409 mmol) in DMF (15 mL) was added 3-mercaptopropionic acid (0.34 mL, 3.9 mmol). The reaction mixture was stirred at room temperature and was monitored by LCMS. At 23 hours, the reaction mixture was poured into water (30 mL) and sonicated. The solids were collected by vacuum filtration rinsing with water. The solids were dissolved in DCM, passed through a hydrophobic frit, dried over Na₂SO₄, and concentrated. ACN (15 mL) was added to the residue. The suspension was sonicated and cooled in an ice bath. Then the solids were collected by vacuum filtration rinsing sparingly with cold ACN. The solids were taken up in ACN (15 mL) and sonicated. Solids were collected by vacuum filtration rinsing with ACN to afford 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (1.333 g, 2.492 mmol, 73.1%) as a white solid. UPLC/ELSD: RT=3.28 min. ¹H NMR (300 MHz, CDCl₃) δ 11.10 (br. s, 1H), 5.46-5.25 (m, 1H), 2.96-2.85 (m, 2H), 2.84-2.74 (m, 2H), 2.74-2.56 (m, 1H), 2.42-2.22 (m, 2H), 2.09-1.75 (m, 5H), 1.75-0.75 (m, 31H), 1.00 (s, 3H), 0.92 (d, J=6.5 Hz, 3H), 0.68 (s, 3H).

### Step 7: di-tert-butyl (((3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-JH-cyclopenta[a]phenanthren-3-yl)disulfaneyl)propanoyl)azanediyl)bis(2-methylbutane-4,2-diyl))dicarbamate

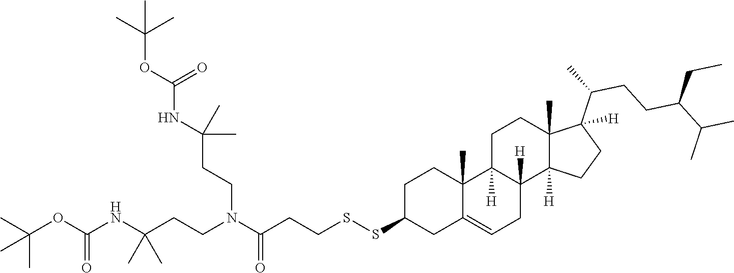

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.110 g, 0.206 mmol), tert-butyl N-[4-({3-[(tert-butoxycarbonyl)amino]-3-methylbutyl}amino)-2-methylbutan-2-yl]carbamate (0.088 g, 0.226 mmol), and triethylamine (0.09 mL, 0.6 mmol) in DCM (1.1 mL) cooled to 0° C. was added 50 wt % propanephosphonic acid anhydride in DCM (0.21 mL, 0.41 mmol) dropwise. The reaction mixture was stirred at room temperature and was monitored by LCMS. At 19 hours, the reaction mixture was diluted with DCM to 10 mL, then washed with 5% aq. NaHCO₃ soln. The aqueous was extracted with DCM (10 mL). The combined organics were washed with water, passed through a hydrophobic frit, dried over Na₂SO₄, and concentrated. The crude material was purified via silica gel chromatography (20-50% EtOAc in hexanes) to afford di-tert-butyl (((3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate (0.139 g, 0.154 mmol, 74.7%) as a clear oil. UPLC/ELSD: RT=3.65 min. MS (ES): m/z=905.77 (M+H)⁺ for C₅₂H₉₃N₃O₅S₂. ¹H NMR (300 MHz, CDCl₃) δ 5.39-5.30 (m, 1H), 4.73 (s, 1H), 4.39 (s, 1H), 3.40-3.18 (m, 4H), 3.04-2.90 (m, 2H), 2.78-2.56 (m, 3H), 2.42-2.22 (m, 2H), 2.11-0.78 (m, 70H), 1.00 (s, 3H), 0.92 (d, J=6.3 Hz, 3H), 0.68 (s, 3H).

### Step 8: N,N-bis(3-amino-3-methylbutyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

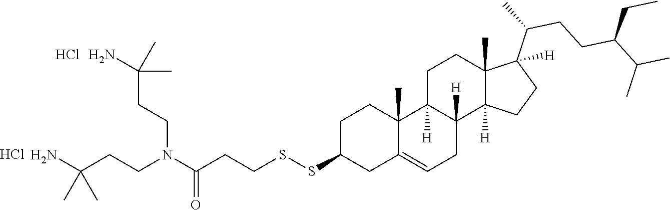

To a solution of di-tert-butyl (((3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate (0.131 g, 0.145 mmol) in DCM (2.0 mL) was added 4 N HCl in dioxane (0.26 mL) dropwise. The reaction mixture was stirred at room temperature and was monitored by LCMS. At 19 hours, the reaction mixture was diluted with MBTE to 30 mL, then was centrifuged (10,000×g for 15 min at 4° C.). The supernatant was drawn off. The solids were suspended in MTBE, then concentrated to afford N,N-bis(3-amino-3-methylbutyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride (0.089 g, 0.109 mmol, 75.4%) as a white solid. UPLC/ELSD: RT=2.25 min. MS (ES): m/z=373.20 [(M+2H)+CH₃CN]²⁺ for C₄₂H₇₇N₃OS₂. ¹H NMR (300 MHz, MeOD) δ 5.46-5.31 (m, 1H), 3.61-3.39 (m, 4H), 3.01-2.91 (m, 2H), 2.88-2.77 (m, 2H), 2.74-2.58 (m, 1H), 2.40-2.24 (m, 2H), 2.16-1.79 (m, 9H), 1.77-0.78 (m, 31H), 1.44 (s, 6H), 1.39 (s, 6H), 1.03 (s, 3H), 0.96 (d, J=6.4 Hz, 3H), 0.73 (s, 3H).

### DK. Compound SA178: N-(3-aminobutyl)-N-(8-aminononyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

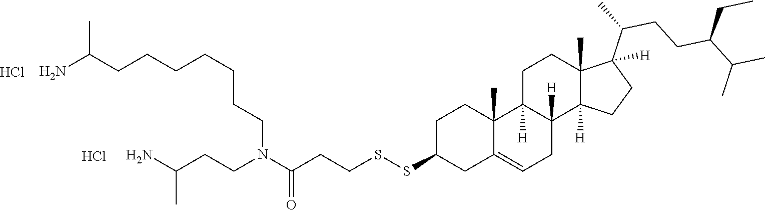

### Step 1: tert-butyl (4-(N-(8-((tert-butoxycarbonyl)amino)nonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamido)butan-2-yl)carbamate

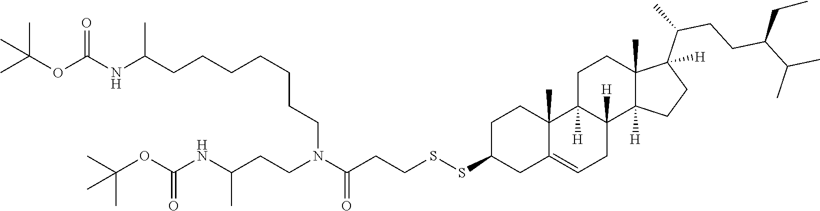

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.100 g, 0.187 mmol), tert-butyl N-[4-({8-[(tert-butoxycarbonyl)amino]nonyl}amino)butan-2-yl]carbamate (0.088 g, 0.206 mmol), and triethylamine (0.08 mL, 0.569 mmol) in DCM (2.5 mL) cooled to 0° C. was added 50 wt % propanephosphonic acid anhydride DCM (0.19 mL, 0.37 mmol) dropwise. The reaction mixture stirred at room temperature. At 19 hours, the reaction mixture was diluted with DCM to 10 mL, then washed with 5% aq. NaHCO₃ soln. The aqueous was extracted with DCM (10 mL). The combined organic layers were passed through a hydrophobic frit, dried over Na₂SO₄, and concentrated. The crude material was purified via silica gel chromatography (20-50% EtOAc in hexanes) to afford tert-butyl (4-(N-(8-((tert-butoxycarbonyl)amino)nonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamido)butan-2-yl)carbamate (0.130 g, 0.137 mmol, 73.5%) as a white foam. UPLC/ELSD: RT=3.65 min. MS (ES): m/z=946.96 (M+H)⁺ for C₅₅H₉₉N₃O₅S₂. ¹H NMR (300 MHz, CDCl₃) δ 5.45-5.29 (m, 1H), 4.70-4.18 (m, 2H), 3.80-3.10 (m, 6H), 3.05-2.87 (m, 2H), 2.83-2.56 (m, 3H), 2.43-2.25 (m, 2H), 2.14-0.75 (m, 74H), 1.00 (s, 3H), 0.92 (d, J=6.4 Hz, 3H), 0.67 (s, 3H).

### Step 2: N-(3-aminobutyl)-N-(8-aminononyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

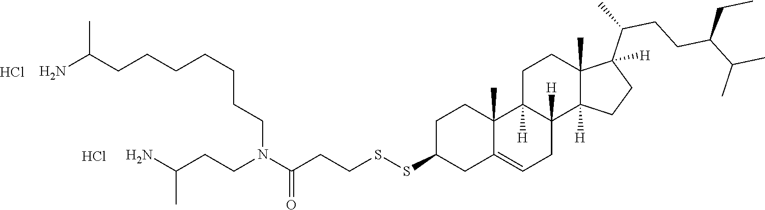

To a stirred solution of tert-butyl (4-(N-(8-((tert-butoxycarbonyl)amino)nonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamido)butan-2-yl)carbamate (0.126 g, 0.133 mmol) in DCM (1.9 mL) was added 4 N HCl in dioxane (0.24 mL) dropwise. The reaction mixture was stirred at room temperature and was monitored by LCMS. At 19 hours, the reaction mixture was diluted with MTBE (30 mL), and the reaction mixture was centrifuged (10,000×g for 15 min at 4° C.). The supernatant was drawn off, and the solids were suspended in MTBE and concentrated to afford N-(3-aminobutyl)-N-(8-aminononyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride (0.101 g, 0.115 mmol, 86.7%). UPLC/ELSD: RT=2.28 min. MS (ES): m/z=373.82 (M+2H)²⁺ for C₄₅H₈₃N₃₀S₂. ¹H NMR (300 MHz, MeOD) δ 5.42-5.33 (m, 1H), 3.76-3.59 (m, 1H), 3.56-3.10 (m, 5H), 3.03-2.57 (m, 5H), 2.42-2.25 (m, 2H), 2.12-0.78 (m, 56H), 1.04 (s, 3H), 0.96 (d, J=6.4 Hz, 3H), 0.73 (s, 3H).

### DL. Compound SA179: N-(3-amino-3-methylbutyl)-N-(8-amino-8-methylnonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

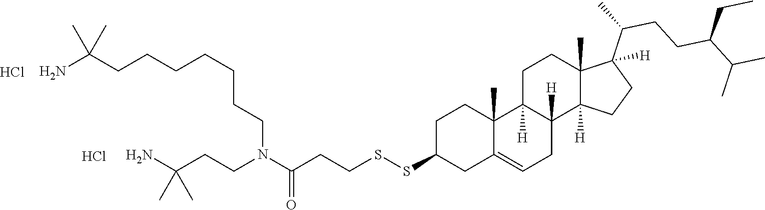

### Step 1: tert-butyl (4-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-N-(8-((((4-methoxybenzyl)oxy)carbonyl)amino)-8-methylnonyl)propanamido)-2-methylbutan-2-yl)carbamate

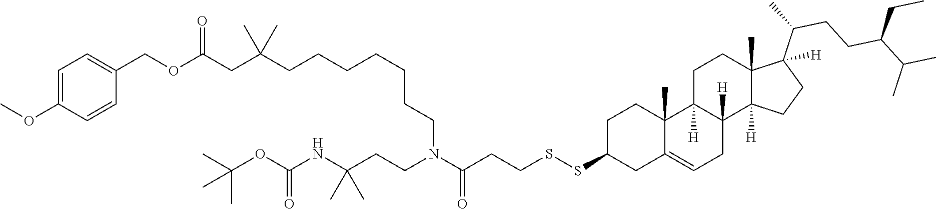

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.102 g, 0.191 mmol), tert-butyl N-(4-{[8-({[(4-methoxyphenyl)methoxy]carbonyl}amino)-8-methylnonyl]amino}-2-methylbutan-2-yl)carbamate (0.113 g, 0.217 mmol), and triethylamine (0.08 mL, 0.6 mmol) in DCM (2.5 mL) cooled to 0° C. was added 50% propanephosphonic acid anhydride in DCM (0.19 mL, 0.37 mmol) dropwise. The reaction mixture was stirred at room temperature and was monitored by LCMS. At 19 hours, the reaction mixture was diluted with DCM to 10 mL, then washed with 5% aq. NaHCO₃ soln. The aqueous was extracted with DCM (10 mL). The combined organics were passed through a hydrophobic frit, dried over Na₂SO₄, and concentrated. The crude material was purified via silica gel chromatography (20-50% EtOAc in hexanes) to afford tert-butyl (4-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-N-(8-((((4-methoxybenzyl)oxy)carbonyl)amino)-8-methylnonyl)propanamido)-2-methylbutan-2-yl)carbamate (0.160 g, 0.154 mmol, 80.8%) as a clear oil. UPLC/ELSD: RT=3.68 min. MS (ES): m/z=1039.59 (M+H)⁺ for C₆₁H₁₀₃N₃O₆S2. ¹H NMR (300 MHz, CDCl₃) δ 7.33-7.27 (m, 2H), 6.92-6.85 (m, 2H), 5.39-5.30 (m, 1H), 4.97 (s, 2H), 4.71-4.35 (m, 2H), 3.81 (s, 3H), 3.38-3.16 (m, 4H), 3.01-2.89 (m, 2H), 2.79-2.55 (m, 3H), 2.39-2.25 (m, 2H), 2.09-0.77 (m, 71H), 1.00 (s, 3H), 0.92 (d, J=6.4 Hz, 3H), 0.68 (s, 3H).

### Step 2: N-(3-amino-3-methylbutyl)-N-(8-amino-8-methylnonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

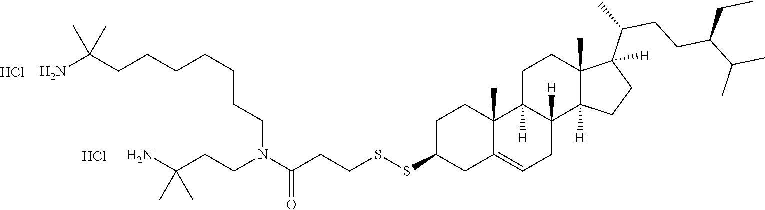

To a solution of tert-butyl (4-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-N-(8-((((4-methoxybenzyl)oxy)carbonyl)amino)-8-methylnonyl)propanamido)-2-methylbutan-2-yl)carbamate (0.154 g, 0.148 mmol) in DCM (2.4 mL) was added 4 N HCl in dioxane (0.26 mL) dropwise. The reaction mixture was stirred at room temperature and was monitored by LCMS. At 19 hours, the reaction mixture was diluted with MTBE to 30 mL, then was centrifuged (10,000×g for 15 min at 4° C.). The supernatant was drawn off. The solids were suspended in MTBE (30 mL), then centrifuged (10,000×g for 15 min at 4° C.). The supernatant was drawn off. The solids were suspended in MBTE and then concentrated to afford N-(3-amino-3-methylbutyl)-N-(8-amino-8-methylnonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride (0.042 g, 0.047 mmol, 31.6%) as a white solid. UPLC/ELSD: RT=2.33 min. MS (ES): m/z=388.12 (M+2H)²⁺ for C₄₇H₈₇N₃OS₂. ¹H NMR (300 MHz, MeOD) δ 5.45-5.33 (m, 1H), 3.52-3.34 (m, 4H), 3.02-2.89 (m, 2H), 2.87-2.74 (m, 2H), 2.73-2.57 (m, 1H), 2.40-2.26 (m, 2H), 2.13-1.79 (m, 7H), 1.37 (s, 6H), 1.33 (s, 6H), 1.77-0.78 (m, 43H), 1.04 (s, 3H), 0.96 (d, J=6.4 Hz, 3H), 0.73 (s, 3H).

### DM. Compound SA180: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(methylamino)butyl)(3-(methylamino)propyl)carbamate dihydrochloride

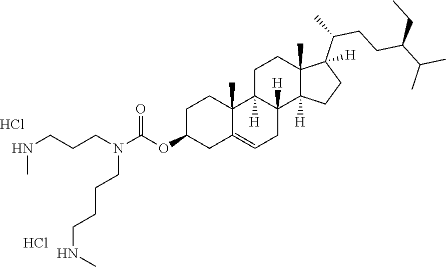

### Step 1: tert-butyl N-methyl-N-[4-(2-nitrobenzenesulfonamido)butyl]carbamate

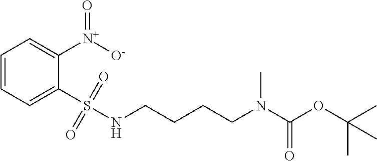 [SMILES: CN(CCCCNS(=O)(=O)c1ccccc1[N+](=O)[O-])C(=O)OC(C)(C)C]

To a stirred solution of tert-butyl N-(4-aminobutyl)-N-methylcarbamate (1.000 g, 4.943 mmol) and triethylamine (0.95 mL, 6.8 mmol) in DCM (15 mL) cooled to 0° C. was added dropwise a solution of 2-nitrobenzenesulfonyl chloride (1.315 g, 5.932 mmol) in DCM (5 mL). After addition, the reaction mixture was stirred at room temperature and was monitored by TLC. At 17 hours, the reaction mixture was cooled to 0° C., then 5% aq. NaHCO₃ soln. (10 mL) was added. After warming to room temperature, the layers were separated. The aqueous layer was extracted with DCM (15 mL). The combined organics were washed with 5% aq. citric acid soln. and water (2×), passed through a hydrophobic frit, dried over Na₂SO₄, and concentrated to afford tert-butyl N-methyl-N-[4-(2-nitrobenzenesulfonamido)butyl]carbamate (1.932 g, quant.) as an amber oil. UPLC/ELSD: RT=0.76 min. MS (ES): m/z=288.09 [(M+H)—(CH₃)₂C═CH₂— CO₂]⁺ for C₁₆H₂₅N₃O₆S. ¹H NMR (300 MHz, CDCl₃) δ 8.18-8.08 (m, 1H), 7.90-7.80 (m, 1H), 7.80-7.68 (m, 2H), 5.34 (br. s, 1H), 3.23-3.06 (m, 4H), 2.79 (s, 3H), 1.60-1.46 (m, 4H), 1.43 (s, 9H).

### Step 2: tert-butyl N-[3-(N-{4-[(tert-butoxycarbonyl)(methyl)amino]butyl}-2-nitrobenzenesulfonamido)propyl]-N-methylcarbamate

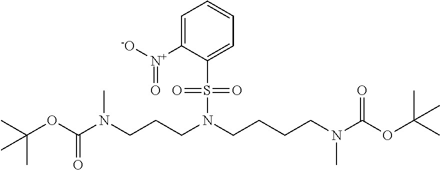

Tert-butyl N-methyl-N-[4-(2-nitrobenzenesulfonamido)butyl]carbamate (0.750 g, 1.94 mmol), tert-butyl N-(3-bromopropyl)-N-methylcarbamate (0.586 g, 2.32 mmol), and potassium carbonate (0.535 g, 3.87 mmol) were combined in DMF (11.25 mL). The reaction mixture was stirred at 80° C. and was monitored by LCMS. At 19 hours, the reaction mixture was filtered rinsing with EtOAc. The filtrate was diluted with EtOAc to 150 mL, then washed with 5% aq. NaHCO₃ soln., water (3×), and brine. The organics were dried over Na₂SO₄ and concentrated. The crude material was purified via silica gel chromatography (20-80% EtOAc in hexanes) to afford tert-butyl N-[3-(N-{4-[(tert-butoxycarbonyl)(methyl)amino]butyl}-2-nitrobenzenesulfonamido)propyl]-N-methylcarbamate (0.836 g, 1.50 mmol, 82.8%) as a white solid. UPLC/ELSD: RT=1.46 min. MS (ES): m/z=559.37 (M+H)⁺ for C₂₅H₄₂N₄O₈S. ¹H NMR (300 MHz, CDCl₃) δ 8.06-7.93 (m, 1H), 7.76-7.55 (m, 3H), 3.42-3.10 (m, 8H), 2.80 (s, 6H), 1.89-1.70 (m, 2H), 1.70-1.46 (m, 4H), 1.44 (s, 18H).

### Step 3: tert-butyl N-[3-({4-[(tert-butoxycarbonyl)(methyl)amino]butyl}amino)propyl]-N-methylcarbamate

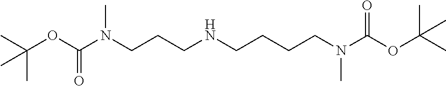 [SMILES: CN(CCCCNCCCN(C)C(=O)OC(C)(C)C)C(=O)OC(C)(C)C]

To a mixture of tert-butyl N-[3-(N-{4-[(tert-butoxycarbonyl)(methyl)amino]butyl}-2-nitrobenzenesulfonamido)propyl]-N-methylcarbamate (0.830 g, 1.49 mmol) and potassium carbonate (0.616 g, 4.46 mmol) in DMF (12.5 mL) was added thiophenol (0.28 mL, 2.7 mmol). The reaction mixture was stirred at room temperature and was monitored by LCMS. At 23 hours, the reaction mixture was filtered rinsing with EtOAc. The filtrate was diluted with EtOAc to 150 mL, then washed with 5% aq. K₂CO₃ (2×), water (3×), and brine. The organics were dried over Na₂SO₄, and then concentrated. The crude material was purified via silica gel chromatography (0-20% (5% conc. aq. NH₄OH in MeOH) in DCM) to afford tert-butyl N-[3-({4-[(tert-butoxycarbonyl)(methyl)amino]butyl}amino)propyl]-N-methylcarbamate (0.477 g, 1.28 mmol, 86.0%) as a yellow oil. UPLC/ELSD: RT=0.45 min. MS (ES): m/z=374.56 (M+H)⁺ for C₁₉H₃₉N₃O₄. ¹H NMR (300 MHz, CDCl₃) δ 3.33-3.11 (m, 4H), 2.83 (s, 6H), 2.70-2.50 (m, 4H), 1.76-1.62 (m, 2H), 1.62-1.37 (m, 22H).

### Step 4: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)(methyl)amino)butyl)(3-((tert-butoxycarbonyl)(methyl)amino)propyl)carbamate

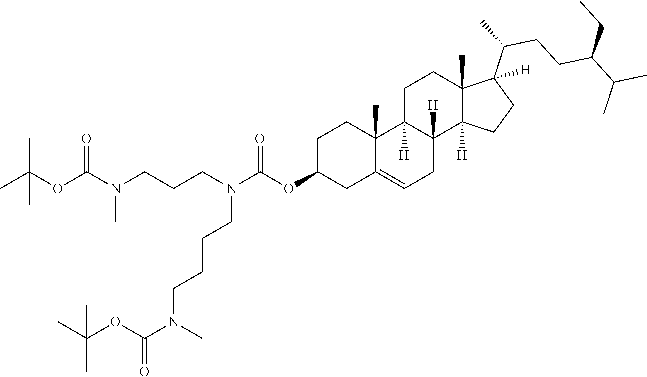

Sitosteryl 4-nitrophenyl carbonate (0.120 g, 0.207 mmol), tert-butyl N-[3-({4-[(tert-butoxycarbonyl)(methyl)amino]butyl}amino)propyl]-N-methylcarbamate (0.077 g, 0.207 mmol), and triethylamine (0.09 mL, 0.6 mmol) were combined in PhMe (1.8 mL). The reaction mixture was stirred at 90° C. and was monitored by LCMS. At 24 hours, the reaction mixture was cooled to room temperature, diluted with DCM to 10 mL, and washed with 5% aq. K₂CO₃ soln. (2×). The aqueous was extracted with DCM (10 mL). The combined organics were passed through a hydrophobic frit, dried over Na₂SO₄, and concentrated. The crude material was purified via silica gel chromatography (0-50% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)(methyl)amino)butyl)(3-((tert-butoxycarbonyl)(methyl)amino)propyl)carbamate (0.152 g, 0.187 mmol, 90.2%) as a clear oil. UPLC/ELSD: RT=3.59 min. MS (ES): m/z=814.88 (M+H)⁺ for C₄₉H₈₇N₃O₆. ¹H NMR (300 MHz, CDCl₃) δ 5.41-5.33 (m, 1H), 4.58-4.42 (m, 1H), 3.32-3.10 (m, 8H), 2.88-2.79 (m, 6H), 2.41-2.20 (m, 2H), 2.09-0.75 (m, 60H), 1.02 (s, 3H), 0.92 (d, J=6.4 Hz, 3H), 0.68 (s, 3H).

### Step 5: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(methylamino)butyl)(3-(methylamino)propyl)carbamate dihydrochloride

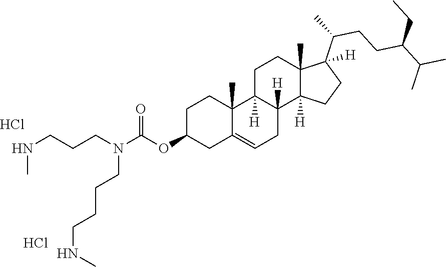

To a solution of (3S,8S,9S,1R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)(methyl)amino)butyl)(3-((tert-butoxycarbonyl)(methyl)amino)propyl)carbamate (0.143 g, 0.176 mmol) in DCM (2.2 mL) was added 4 N HCl in dioxane (0.31 mL). The reaction mixture was stirred at room temperature and was monitored by LCMS. At 18 hours, the reaction mixture was diluted with MTBE to 20 mL, then centrifuged (10,000×g for 30 min at 4° C.). The supernatant was drawn off. The solids were rinsed with MTBE, suspended in MTBE, and concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(methylamino)butyl)(3-(methylamino)propyl)carbamate dihydrochloride (0.102 g, 0.131 mmol, 74.8%) as a white solid. UPLC/ELSD: RT=2.03 min. MS (ES): m/z=328.45 [(M+2H)+CH₃CN]²⁺ for C₃₉H₇₁N₃O₂. ¹H NMR (300 MHz, MeOD) δ 5.44-5.33 (m, 1H), 4.52-4.37 (m, 1H), 3.47-3.33 (m, 4H), 3.10-2.90 (m, 4H), 2.78-2.63 (m, 6H), 2.44-2.27 (m, 2H), 2.19-0.78 (m, 42H), 1.06 (s, 3H), 0.96 (d, J=6.4 Hz, 3H), 0.73 (s, 3H).

### DN. Compound SA181: N-(3-amino-3-ethylpentyl)-N-(4-((3-amino-3-ethylpentyl)amino)butyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide trihydrochloride

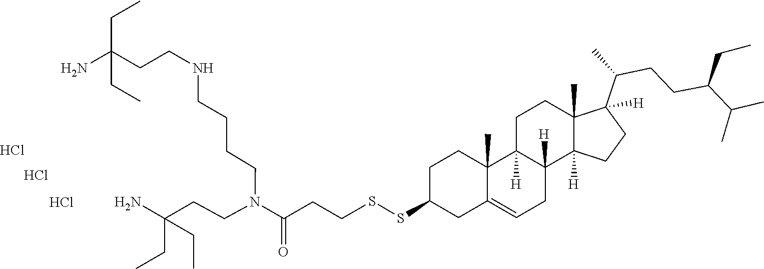

### Step 1: tert-Butyl (6,6,17-triethyl-9-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)-2,2-dimethyl-4-oxo-3-oxa-5,9,14-triazanonadecan-17-yl)carbamate

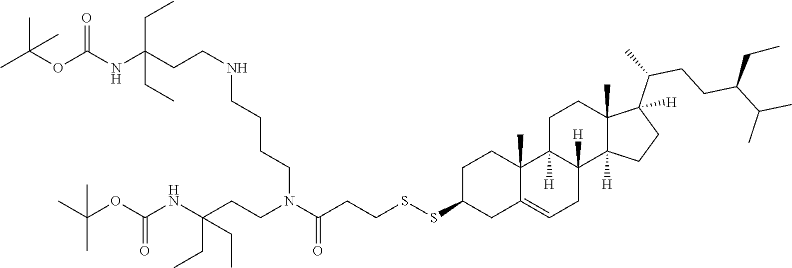

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.150 g, 0.280 mmol), tert-butyl N-(1-{[4-({3-[(tert-butoxycarbonyl)amino]-3-ethylpentyl}amino)butyl]amino}-3-ethylpentan-3-yl)carbamate (0.217 g, 0.421 mmol), and triethylamine (0.14 mL, 1.0 mmol) in DCM (3.75 mL) was added 50 wt % propanephosphonic acid anhydride in DCM (0.29 mL, 0.56 mmol) dropwise. The reaction mixture stirred at rt and was monitored by LCMS. At 17 h, the reaction mixture was diluted with DCM to 15 mL, then washed with 5% aq. NaHCO₃ solution. The aqueous was extracted with DCM (2×15 mL). The combined organics were dried over Na₂SO₄ and concentrated. The crude material was purified via silica gel chromatography (3:2 EtOAc/hexanes, then 0-20% (5% conc. aq. NH₄OH in MeOH) in DCM) to afford tert-butyl (6,6,17-triethyl-9-(3-(((3 S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)-2,2-dimethyl-4-oxo-3-oxa-5,9,14-triazanonadecan-17-yl)carbamate (0.110 g, 0.107 mmol, 38.2%) as a white foam. UPLC/ELSD: RT=3.01 min. MS (ES): m/z=1032.19 (M+H)⁺ for C₆₀H₁₁₀N₄O₅S₂. ¹H NMR (300 MHz, CDCl₃) δ 5.44-5.32 (m, 1H), 4.89 (br. s, 1H), 4.53-4.16 (m, 1H), 3.43-3.14 (m, 4H), 3.11-2.86 (m, 2H), 2.84-2.43 (m, 5H), 2.43-2.22 (m, 2H), 2.10-0.73 (m, 84H), 0.99 (s, 3H), 0.92 (d, J=6.6 Hz, 3H), 0.67 (s, 3H).

### Step 2: N-(3-amino-3-ethylpentyl)-N-(4-((3-amino-3-ethylpentyl)amino)butyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide trihydrochloride

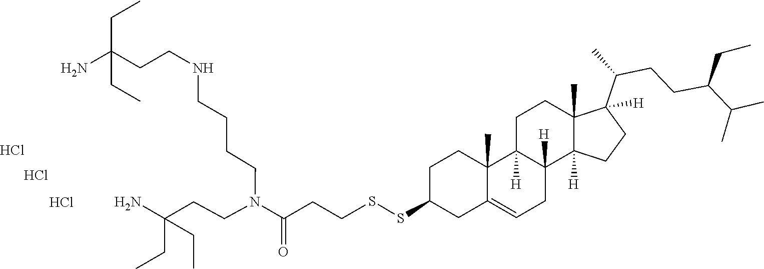

To a stirred solution of tert-butyl (6,6,17-triethyl-9-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)-2,2-dimethyl-4-oxo-3-oxa-5,9,14-triazanonadecan-17-yl)carbamate (0.103 g, 0.100 mmol) in DCM (2.1 mL) was added 4 N HCl in dioxane (0.17 mL, 0.68 mmol). The reaction mixture stirred at rt and was monitored by LCMS. At 16 h, the reaction mixture was diluted with MTBE to 20 mL, then centrifuged (10,000×g for 30 min at 4° C.). The supernatant was drawn off, and the solids were rinsed with MTBE. The solids were suspended in MTBE, then concentrated to afford N-(3-amino-3-ethylpentyl)-N-(4-((3-amino-3-ethylpentyl)amino)butyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide trihydrochloride (0.092 g, 0.088 mmol, 87.8%) as a white solid. UPLC/ELSD: RT=2.17 min. MS (ES): m/z=416.72 (M+2H)²⁺ for CsoH₉₄N₄OS₂. ¹H NMR (300 MHz, MeOD) δ 5.41-5.33 (m, 1H), 3.58-3.37 (m, 4H), 3.19-3.06 (m, 4H), 3.03-2.92 (m, 2H), 2.88-2.78 (m, 2H), 2.73-2.57 (m, 1H), 2.39-2.27 (m, 2H), 2.20-0.77 (m, 70H), 0.72 (s, 3H).

### DO. Compound SA182: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride

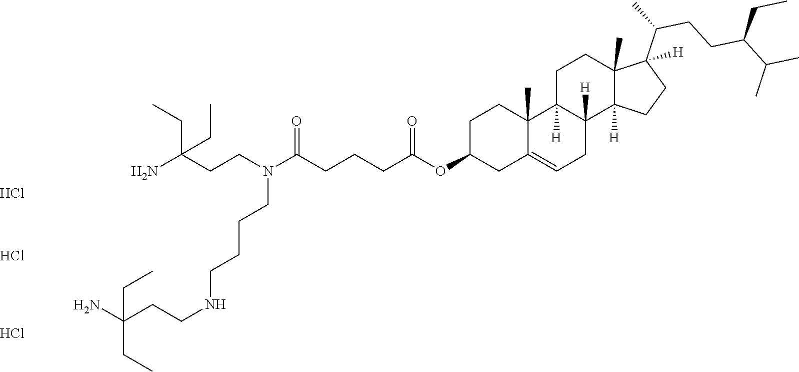

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate

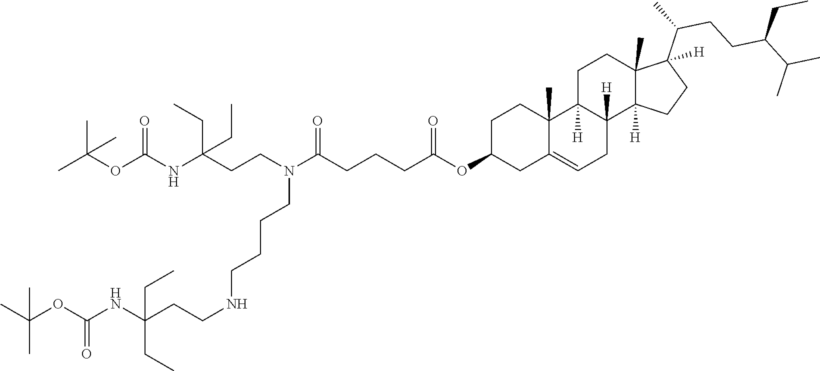

To a stirred solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.150 g, 0.284 mmol), tert-butyl N-(1-{[4-({3-[(tert-butoxycarbonyl)amino]-3-ethylpentyl}amino)butyl]amino}-3-ethylpentan-3-yl)carbamate (0.219 g, 0.425 mmol), and triethylamine (0.14 mL, 1.0 mmol) in DCM (3.75 mL) was added 50 wt % propanephosphonic acid anhydride in DCM (0.29 mL, 0.56 mmol) dropwise. The reaction mixture stirred at rt and was monitored by LCMS. At 17 h, the reaction mixture was diluted with DCM to 15 mL, then washed with 5% aq. NaHCO₃ solution. The aqueous was extracted with DCM (2×15 mL). The combined organics were passed through a hydrophobic frit, dried over Na₂SO₄, and concentrated. The crude material was purified via silica gel chromatography (3:2 EtOAc/hexanes, then 0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate (0.123 g, 0.120 mmol, 42.3%) as a white foam. UPLC/ELSD: RT=2.96 min. MS (ES): m/z=1026.39 (M+H)⁺ for C₆₂H₁₁₂N₄O₇. ¹H NMR (300 MHz, CDCl₃) δ 5.41-5.31 (m, 1H), 5.00-4.70 (m, 1H), 4.69-4.51 (m, 1H), 4.51-4.16 (m, 1H), 3.37-3.11 (m, 4H), 3.11-2.53 (m, 4H), 2.43-2.21 (m, 6H), 2.12-0.72 (m, 84H), 1.01 (s, 3H), 0.92 (d, J=6.4 Hz, 3H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride

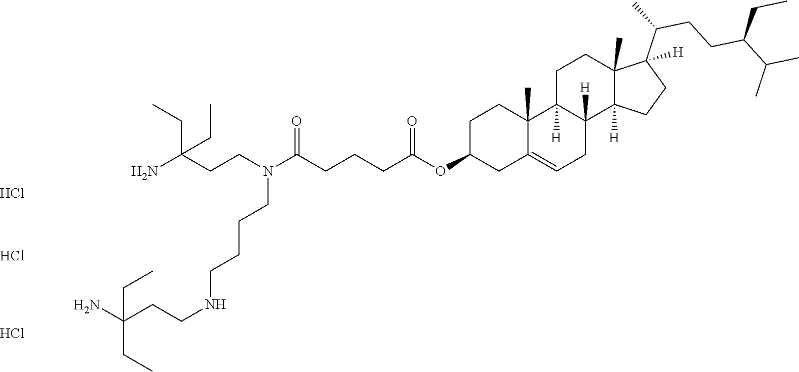

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate (0.119 g, 0.116 mmol) in DCM (2.4 mL) was added 4 N HCl in dioxane (0.20 mL). The reaction mixture stirred at rt and was monitored by LCMS. At 16 h, the reaction mixture was diluted with MTBE to 20 mL, then centrifuged (10,000×g for 30 min at 4° C.). The supernatant was drawn off, and the solids were rinsed with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride (0.105 g, 0.092 mmol, 79.5%) as an off-white solid. UPLC/ELSD: RT=2.09 min. MS (ES): m/z=413.39 (M+2H)²⁺ for Cs₂H₉₆N₄O₃. ¹H NMR (300 MHz, MeOD) δ 5.42-5.35 (m, 1H), 4.62-4.46 (m, 1H), 3.57-3.36 (m, 4H), 3.18-3.01 (m, 4H), 2.56-2.25 (m, 6H), 2.20-0.77 (m, 72H), 0.72 (s, 3H).

### DP. Compound SA183: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride

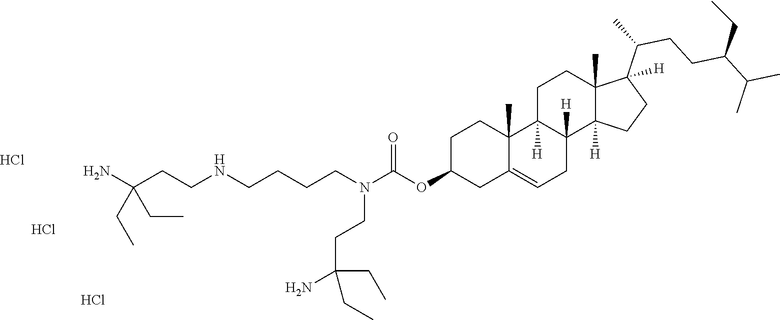

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-ethyl-3-(((neopentyloxy)carbonyl)amino)pentyl)amino)butyl)carbamate

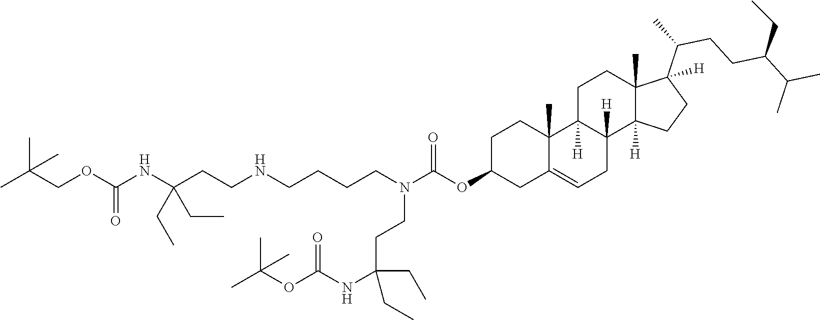

Sitosteryl 4-nitrophenyl carbonate (0.140 g, 0.241 mmol), tert-butyl N-(1-{[4-({3-[(tert-butoxycarbonyl)amino]-3-ethylpentyl}amino)butyl]amino}-3-ethylpentan-3-yl)carbamate (0.187 g, 0.362 mmol), and triethylamine (0.14 mL, 1.0 mmol) were combined in PhMe (3.5 mL). The reaction mixture stirred at 100° C. and was monitored by LCMS. At 16 h, the reaction mixture was cooled to rt, diluted with DCM to 15 mL, and then washed with 5% aq. K₂CO₃ solution. (2×). The combined washes were extracted with DCM (2×15 mL). The combined organics were passed through a hydrophobic frit, dried over Na₂SO₄, and concentrated. The crude material was purified via silica gel chromatography (0-20% (5% conc. aq. NH₄OH in MeOH) in DCM). The material was further purified via silica gel chromatography (1:1 EtOAc/hexanes, then 0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-ethyl-3-(((neopentyloxy)carbonyl)amino)pentyl)amino)butyl)carbamate (0.097 g, 0.10 mmol, 41.5%) as a white foam. UPLC/ELSD: RT=2.95 min. MS (ES): m/z=956.34 (M+H)⁺ for C₅₈H₁₀₆N₄O₆. ¹H NMR (300 MHz, CDCl₃) δ 5.44-5.29 (m, 1H), 5.17-4.91 (m, 1H), 4.59-4.41 (m, 1H), 4.35-4.06 (m, 1H), 3.35-3.02 (m, 4H), 2.70-2.53 (m, 4H), 2.46-2.17 (m, 2H), 2.08-0.73 (m, 82H), 1.01 (s, 3H), 0.92 (d, J=6.5 Hz, 3H), 0.68 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride

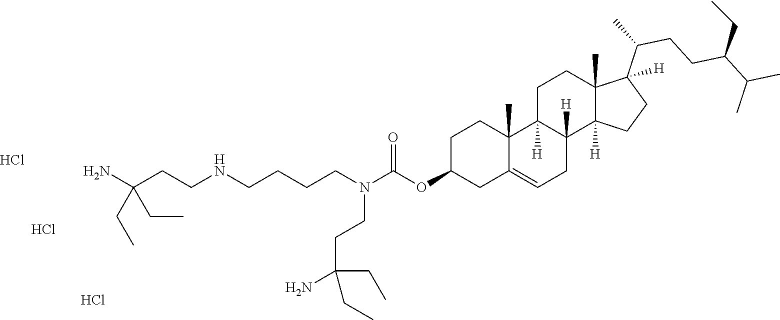

To a stirred solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-ethyl-3-(((neopentyloxy)carbonyl)amino)pentyl)amino)butyl)carbamate (0.094 g, 0.098 mmol) in DCM (1.9 mL) was added 4 N HCl in dioxane (0.17 mL). The reaction mixture stirred at rt and was monitored by LCMS. At 16 h, the reaction mixture was diluted with MTBE to 20 mL and then centrifuged (10,000×g for 30 min at 4° C.). The supernatant was drawn off, and the solids were rinsed with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride (0.083 g, 0.089 mmol, 90.1%) as a white solid. UPLC/ELSD: RT=1.99 min. MS (ES): m/z=378.75 (M+2H)²⁺ for C₄₈H₉₀N₄O₂. ¹H NMR (300 MHz, MeOD) δ 5.45-5.36 (m, 1H), 4.52-4.35 (m, 1H), 3.44-3.34 (m, 4H), 3.17-3.05 (m, 4H), 2.42-2.31 (m, 2H), 2.16-0.78 (m, 70H), 0.73 (s, 3H).

### DQ. Compound SA184: N-(3-amino-3-ethylpentyl)-N-(4-((3-amino-3-ethylpentyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

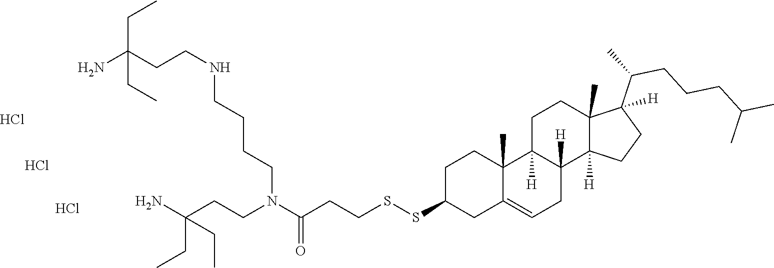

### Step 1: tert-butyl (14-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-6,6,17-triethyl-2,2-dimethyl-4-oxo-3-oxa-5,9,14-triazanonadecan-17-yl)carbamate

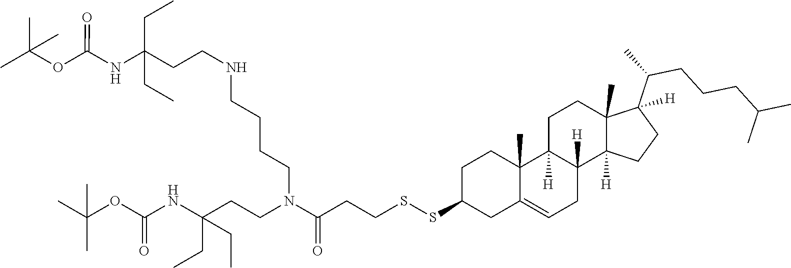

To a stirred solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.140 g, 0.276 mmol), tert-butyl N-(1-{[4-({3-[(tert-butoxycarbonyl)amino]-3-ethylpentyl}amino)butyl]amino}-3-ethylpentan-3-yl)carbamate (0.213 g, 0.414 mmol), and triethylamine (0.14 mL, 1.0 mmol) in DCM (3.5 mL) was added 50 wt % propanephosphonic acid anhydride in DCM (0.24 mL, 0.468 mmol) dropwise. The reaction mixture stirred at rt and was monitored by LCMS. At 3 h, the reaction mixture was diluted with DCM to 15 mL, then washed with 5% aq. NaHCO₃ solution. The aqueous was extracted with DCM (2×15 mL). The combined organics were passed through a hydrophobic frit, dried over Na₂SO₄, and concentrated. The crude material was purified via silica gel chromatography (0-20% (5% conc. aq. NH₄OH in MeOH) in DCM) to afford tert-butyl (14-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-6,6,17-triethyl-2,2-dimethyl-4-oxo-3-oxa-5,9,14-triazanonadecan-17-yl)carbamate (0.097 g, 0.097 mmol, 35.1%) as a clear, yellow oil. UPLC/ELSD: RT=2.95 min. MS (ES): m/z=1004.81 (M+H)⁺ for C₅₈H₁₀₆N₄O₅S2. ¹H NMR (300 MHz, CDCl₃) δ 5.39-5.31 (m, 1H), 5.03-4.74 (m, 1H), 4.52-4.25 (m, 1H), 3.39-3.10 (m, 4H), 3.10-2.85 (m, 3H), 2.85-2.42 (m, 6H), 2.42-2.22 (m, 2H), 2.09-0.72 (m, 78H), 0.99 (s, 3H), 0.91 (d, J=6.5 Hz, 3H), 0.67 (s, 3H).

### Step 2: N-(3-amino-3-ethylpentyl)-N-(4-((3-amino-3-ethylpentyl)amino)butyl)-3-(((3S,8S,9S,10R,13R,14S,17R)-10,13-dimethyl-17-((R)-6-methylheptan-2-yl)-2,3,4, 7, 8,9,10,11,12,13,14,15,16,17-tetradecahydro-1H-cyclopenta[a]phenanthren-3-yl)disulfaneyl)propanamide trihydrochloride

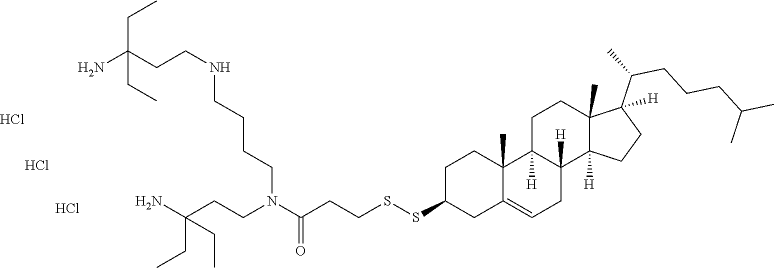

To a stirred solution of tert-butyl (14-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-6,6,17-triethyl-2,2-dimethyl-4-oxo-3-oxa-5,9,14-triazanonadecan-17-yl)carbamate (0.091 g, 0.091 mmol) in DCM (2.3 mL) was added 4 N HCl in dioxane (0.16 mL). The reaction mixture stirred at rt and was monitored by LCMS. At 16 h, the reaction mixture was diluted with MTBE to 20 mL, then centrifuged (10,000×g for 30 min at 4° C.). The supernatant was drawn off. The solids were rinsed with MTBE, then suspended in MTBE and concentrated to afford N-(3-amino-3-ethylpentyl)-N-(4-((3-amino-3-ethylpentyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride (0.080 g, 0.080 mmol, 88.4%) as a white solid. UPLC/ELSD: RT=2.04 min. MS (ES): m/z=402.54 (M+2H)²⁺ for C₄₈H₉₀N₄OS₂. ¹H NMR (300 MHz, MeOD) δ 5.45-5.31 (m, 1H), 3.58-3.36 (m, 4H), 3.19-3.04 (m, 4H), 3.03-2.89 (m, 2H), 2.89-2.76 (m, 2H), 2.73-2.55 (m, 1H), 2.42-2.23 (m, 2H), 2.18-0.96 (m, 57H), 0.94 (d, J=6.5 Hz, 3H), 0.88 (d, J=6.6 Hz, 6H), 0.72 (s, 3H).

### DR. Compound SA185: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride

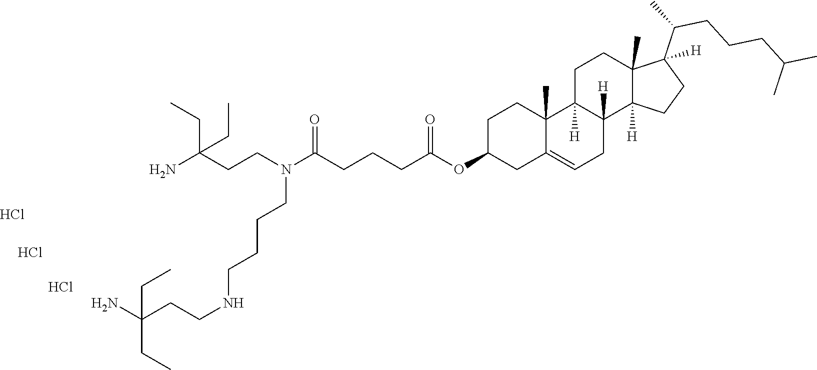

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate

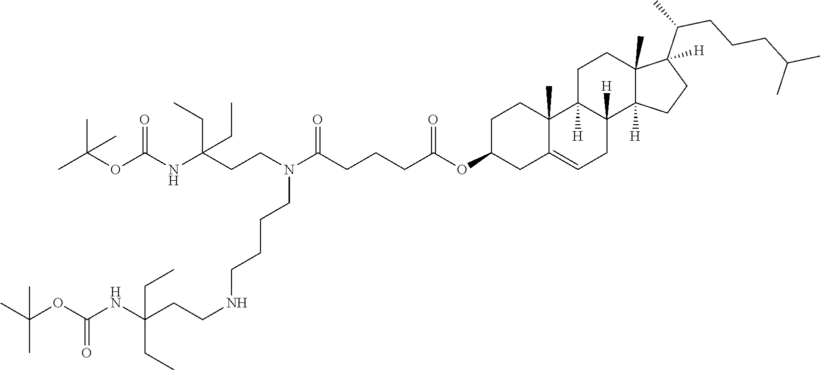

To a stirred solution of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (0.140 g, 0.28 mmol), tert-butyl N-(1-{[4-({3-[(tert-butoxycarbonyl)amino]-3-ethylpentyl}amino)butyl]amino}-3-ethylpentan-3-yl)carbamate (0.216 g, 0.419 mmol), and triethylamine (0.14 mL, 1.0 mmol) in DCM (3.5 mL) was added 50 wt % propanephosphonic acid anhydride in DCM (0.28 mL, 0.546 mmol) dropwise. The reaction mixture stirred at rt and was monitored by LCMS. At 3 h, the reaction mixture was diluted with DCM to 15 mL, then washed with 5% aq. NaHCO₃ solution. The aqueous was extracted with DCM (2×15 mL). The combined organics were passed through a hydrophobic frit, dried over Na₂SO₄, and concentrated. The crude material was purified via silica gel chromatography (0-14% (5% conc. aq. NH₄OH in MeOH) in DCM) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate (0.071 g, 0.071 mmol, 25.4%) as a clear, yellow oil. UPLC/ELSD: RT=2.89 min. MS (ES): m/z=998.15 (M+H)⁺ for C_{W}H₁₀₈N₄O₇. ¹H NMR (300 MHz, CDCl₃) δ 5.43-5.31 (m, 1H), 5.11-4.87 (m, 1H), 4.68-4.51 (m, 1H), 4.40-4.12 (m, 1H), 3.37-3.12 (m, 4H), 2.72-2.51 (m, 4H), 2.43-2.21 (m, 6H), 2.07-0.74 (m, 80H), 1.01 (s, 3H), 0.91 (d, J=6.5 Hz, 3H), 0.67 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride

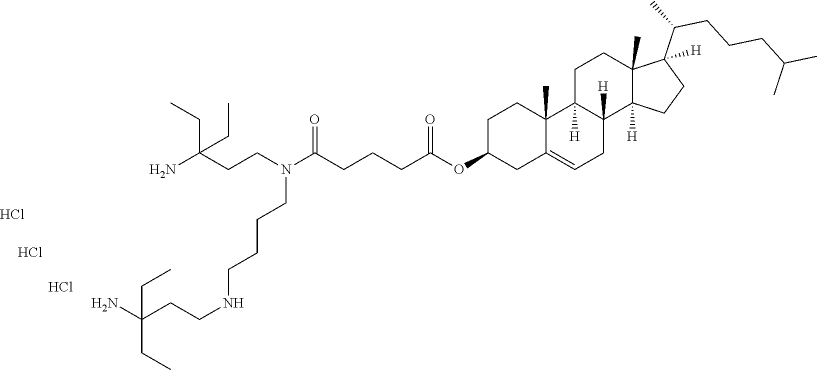

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate (0.067 g, 0.067 mmol) in DCM (1.8 mL) was added 4 N HCl in dioxane (0.12 mL). The reaction mixture stirred at rt and was monitored by LCMS. At 16 h, the reaction mixture was diluted with MTBE to 20 mL, then centrifuged (10,000×g for 30 min at 4° C.). The supernatant was drawn off. The solids were rinsed with MTBE, then suspended in MTBE and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride (0.061 g, 0.066 mmol, 97.5%) as a white solid. UPLC/ELSD: RT=1.96 min. MS (ES): m/z=399.58 (M+2H)²⁺ for C₅₀H₉₂N₄O₃. ¹H NMR (300 MHz, MeOD) δ 5.45-5.33 (m, 1H), 4.62-4.46 (m, 1H), 3.56-3.36 (m, 4H), 3.18-3.03 (m, 4H), 2.55-2.25 (m, 6H), 2.18-0.98 (m, 59H), 0.95 (d, J=6.5 Hz, 3H), 0.88 (d, J=6.6 Hz, 6H), 0.73 (s, 3H).

### DS. Compound SA186: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(2-(3-aminocyclobutyl)ethyl)carbamate dihydrochloride

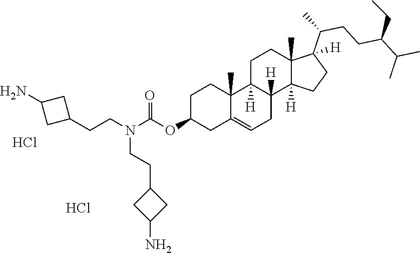

### Step 1: Tert-butyl N-{3-[2-(2-nitrobenzenesulfonamido)ethyl]cyclobutyl}carbamate

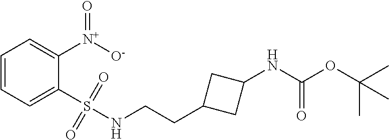 [SMILES: CC(C)(C)OC(=O)NC1CC(CCNS(=O)(=O)c2ccccc2[N+](=O)[O-])C1]

To a solution of tert-butyl N-[3-(2-aminoethyl)cyclobutyl]carbamate (1.000 g, 4.666 mmol) and triethylamine (0.90 mL, 6.5 mmol) in DCM (15 mL) cooled to 0° C. was added 2-nitrobenzenesulfonyl chloride (1.241 g, 5.599 mmol) in DCM (5 mL) dropwise, then the reaction mixture stirred at rt. The reaction was monitored by TLC. At 17 h, the reaction mixture was cooled to 0° C., then 5% aq. NaHCO₃ soln. (10 mL) was added. After warming to rt, the layers were separated. The aq. was extracted with DCM (15 mL). The combined organics were washed with 5% aq. citric acid soln. and water (2×), passed through a hydrophobic frit, dried over Na₂SO₄, and concentrated. The crude material was purified via silica gel chromatography (20-60% EtOAc in hexanes) to afford tert-butyl N-{3-[2-(2-nitrobenzenesulfonamido)ethyl]cyclobutyl}carbamate (1.287 g, 3.222 mmol, 69.1%) as a yellow solid and as a mixture of geometric isomers. ¹H NMR (300 MHz, CDCl₃, reported as observed in spectrum): δ 8.22-8.04 (m, 0.96H), 7.93-7.81 (m, 1.01H), 7.81-7.63 (m, 1.97H), 5.24 (t, J=6.0 Hz, 0.95H), 4.83-4.38 (m, 0.90H), 4.22-3.75 (m, 1.04H), 3.17-2.92 (m, 2.00H), 2.56-2.36 (m, 1.32H), 2.29-2.12 (m, 0.39H), 2.08-1.78 (m, 2.19H), 1.78-1.55 (m, 2.44H), 1.54-1.33 (m, 10.43H). UPLC/ELSD: RT=0.77 min. MS (ES): m/z=344.11 [(M+H)—(CH₃)₂C═CH₂]⁺ for C₁₇H₂₅N₃O₆S.

### Step 2: tert-butyl N-(3-[2-[N-(2-[3-[(tert-butoxycarbonyl)amino]cyclobutyl]ethyl)-2-nitrobenzenesulfonamido]ethyl]cyclobutyl)carbamate

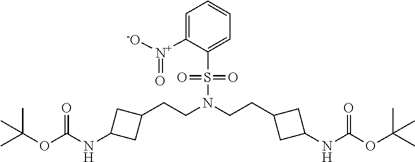

Tert-butyl N-{3-[2-(2-nitrobenzenesulfonamido)ethyl]cyclobutyl}carbamate (1.050 g, 2.629 mmol), tert-butyl N-[3-(2-bromoethyl)cyclobutyl]carbamate (0.877 g, 3.15 mmol), potassium carbonate (0.727 g, 5.26 mmol), and potassium iodide (0.218 g, 1.31 mmol) were combined in propionitrile (16 mL) in a sealed tube. The reaction mixture was heated at 150° C. via microwave irradiation for 4 h. Reaction was monitored by LCMS. The reaction mixture was filtered rinsing with ACN. The filtrate was concentrated, taken up in DCM (75 mL), and then washed with 5% aq. NaHCO₃ soln. The organics were passed through a hydrophobic frit, dried over Na₂SO₄, and concentrated. The crude material was purified via silica gel chromatography (30-60% EtOAc in hexanes) to afford tert-butyl N-(3-{2-[N-(2-{3-[(tert-butoxycarbonyl)amino]cyclobutyl}ethyl)-2-nitrobenzenesulfonamido]ethyl}cyclobutyl)carbamate (1.459 g, 2.445 mmol, 93.0%) as a yellow oil and as a mixture of geometric isomers. ¹H NMR (300 MHz, CDCl₃, reported as observed in spectrum): δ 8.05-7.88 (m, 1.00H), 7.76-7.51 (m, 2.93H), 4.79-4.43 (m, 1.85H), 3.92 (br. s, 1.51H), 3.29-2.98 (m, 3.96H), m, 2.57-2.31 (m, 2.80H), 2.19-1.51 (br. m, 13.81H), 1.51-1.30 (m, 20.03H). UPLC/ELSD: RT=1.50 min. MS (ES): m/z=597.22 [M+H]⁺ for C₂₈H₄₄N₄O₈S.

### Step 3: tert-butyl N-(3-{2-[(2-{3-[(tert-butoxycarbonyl)amino]cyclobutyl}ethyl)amino]ethyl}cyclobutyl)carbamate

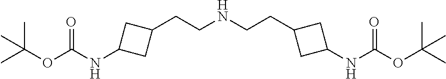 [SMILES: CC(C)(C)OC(=O)NC1CC(CCNCCC2CC(NC(=O)OC(C)(C)C)C2)C1]

To a stirred solution of tert-butyl N-(3-{2-[N-(2-{3-[(tert-butoxycarbonyl)amino]cyclobutyl}ethyl)-2-nitrobenzenesulfonamido]ethyl}cyclobutyl)carbamate (1.447 g, 2.425 mmol) and potassium carbonate (1.005 g, 7.275 mmol) in DMF (21 mL) was added thiophenol (0.45 mL, 4.4 mmol). The reaction mixture stirred at rt and was monitored by LCMS. At 23 h, the reaction mixture was filtered rinsing with copious EtOAc. The filtrate was diluted with EtOAc to 150 mL, then washed with 5% aq. NaHCO₃ soln. (2×), water (3×), and brine. The organics were dried over Na₂SO₄ and concentrated. The crude material was purified via silica gel chromatography (0-17% (5% conc. aq. NH₄OH in MeOH) in DCM) to afford tert-butyl N-(3-{2-[(2-{3-[(tert-butoxycarbonyl)amino]cyclobutyl}ethyl)amino]ethyl}cyclobutyl)carbamate (0.668 g, 1.62 mmol, 66.9%) as a yellow oil and as a mixture of geometric isomers. ¹H NMR (300 MHz, CDCl₃, reported as observed in spectrum): δ 4.87-4.26 (m, 2.26H), 4.26-4.05 (m, 0.67H), 4.05-3.76 (m, 1.54H), 2.64-2.36 (m, 7.07H), 2.35-1.77 (m, 5.12H), 1.71-1.28 (m, 23.32H), 0.88 (br. s, 1.00H). UPLC/ELSD: RT=0.41 min. MS (ES): m/z=412.28 [M+H]⁺ for C₂₂H₄₁N₃O₄.

### Step 4: tert-butyl (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(2-(3-((tert-butoxycarbonyl)amino)cyclobutyl)ethyl)carbamate

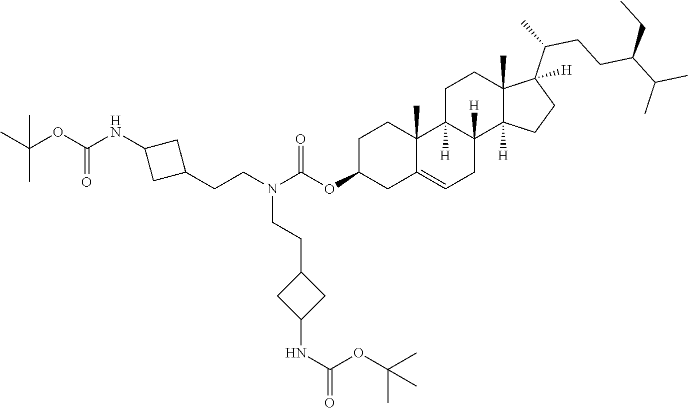

Sitosteryl 4-nitrophenyl carbonate (0.100 g, 0.172 mmol), tert-butyl N-(3-{2-[(2-{3-[(tert-butoxycarbonyl)amino]cyclobutyl}ethyl)amino]ethyl}cyclobutyl)carbamate (0.085 g, 0.21 mmol), triethylamine (0.05 mL, 0.4 mmol), and DMAP (cat.) were combined in PhMe (1.5 mL). The reaction mixture stirred at 90° C. and was monitored by LCMS. At 18 h, the reaction mixture was cooled to rt, diluted with DCM (15 mL) and washed with 5% aq. K₂CO₃ soln. The aqueous was extracted with DCM (10 mL). The combined organics were passed through a hydrophobic frit, dried over Na₂SO₄, and concentrated. The crude material was purified via silica gel chromatography (0-50% hexanes in EtOAc) to afford tert-butyl (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(2-(3-((tert-butoxycarbonyl)amino)cyclobutyl)ethyl)carbamate (0.162 g, 0.171 mmol, quant.) as a clear oil and as a mixture of geometric isomers. ¹H NMR (300 MHz, CDCl₃, reported as observed in spectrum): δ 5.45-5.31 (m, 1.00H), 4.77-4.38 (m, 2.92H), 4.18 (br. s, 0.50H), 3.95 (br. s, 1.57H), 3.19-2.95 (m, 3.87H), 2.57-2.18 (m, 5.02H), 2.18-0.72 (br. m, 84.47H), 0.68 (s, 2.86H). UPLC/ELSD: RT=3.52 min. MS (ES): m/z=852.74 [M+H]⁺ for C₅₂H₈₉N₃₀₆.

### Step 5: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(2-(3-aminocyclobutyl)ethyl)carbamate dihydrochloride

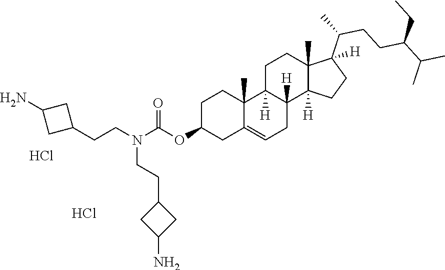

To a stirred solution of tert-butyl (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(2-(3-((tert-butoxycarbonyl)amino)cyclobutyl)ethyl)carbamate (0.139 g, 0.163 mmol) in DCM (2.2 mL) was added 4 N HCl in dioxane (0.41 mL). The reaction mixture stirred at rt and was monitored by LCMS. At 19 h, the reaction mixture was diluted with MTBE to 20 mL, then centrifuged (10,000×g for 15 min at 4° C.). The supernatant was drawn off, and the solids were rinsed with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(2-(3-aminocyclobutyl)ethyl)carbamate dihydrochloride (0.105 g, 0.138 mmol, 84.6%) as a white solid and as mixture of geometric isomers. ¹H NMR (300 MHz, CD₃OD, reported as observed in spectrum): δ 5.45-5.29 (m, 1.00H), 3.96-3.74 (m, 0.76H), 3.74-3.50 (m, 1.67H), 3.30-3.23 (m, 2.28H), 3.04-2.86 (m, 2.17H), 2.84-2.72 (m, 2.05H), 2.71-0.78 (br. m, 62.42H), 0.77-0.69 (m, 2.98H). UPLC/ELSD: RT=2.07 min. MS (ES): m/z=347.56 [(M+2H)+CD₃CN]²⁺ for C₄₂H₇₃N₃O₂.

### DT. Compound SA187: N,N-bis(2-(3-aminocyclobutyl)ethyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

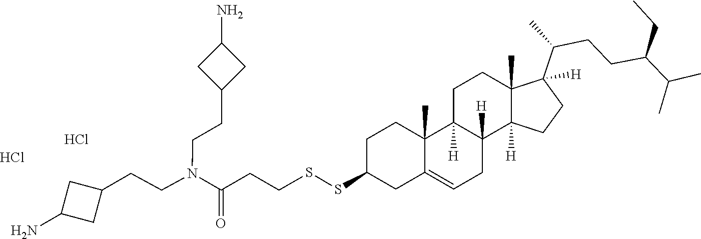

### Step 1: di-tert-butyl ((((3-(((3S,8S,9S,10R,13R,14S,17R)-17-((2R,5R)-5-ethyl-6-methylheptan-2-yl)-10,13-dimethyl-2,3,4,7, 8,9,10,11,12,13,14,15,16,17-tetradecahydro-1H-cyclopenta[a]phenanthren-3-yl)disulfaneyl)propanoyl)azanediyl)bis(ethane-2,1-diyl))bis(cyclobutane-3,1-diyl))dicarbamate

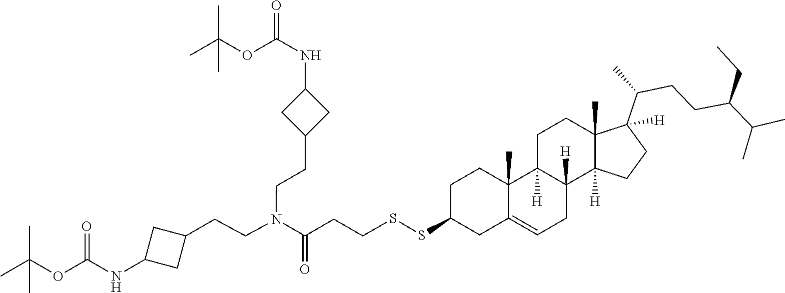

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.120 g, 0.224 mmol), tert-butyl N-(3-{2-[(2-{3-[(tert-butoxycarbonyl)amino]cyclobutyl}ethyl)amino]ethyl}cyclobutyl)carbamate (0.102 g, 0.247 mmol), and triethylamine (0.10 mL, 0.71 mmol) cooled to 0° C. was added 50 wt % propanephosphonic acid anhydride in DCM (0.23 mL, 0.45 mmol) dropwise. The reaction mixture stirred at rt and was monitored by LCMS. At 18 h, the reaction mixture was diluted with DCM (15 mL) and washed with 5% aq. NaHCO₃ soln. The aqueous was extracted with DCM (10 mL). The combined organics were washed with water, passed through a hydrophobic frit, dried over Na₂SO₄, and concentrated. The crude material was purified via silica gel chromatography (25-65% EtOAc in hexanes) to afford di-tert-butyl ((((3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)azanediyl)bis(ethane-2,1-diyl))bis(cyclobutane-3,1-diyl))dicarbamate (0.195 g, 0.210 mmol, 93.6%) as a clear oil and as a mixture of geometric isomers. ¹H NMR (300 MHz, CDCl₃, reported as observed in spectrum): δ 5.42-5.25 (m, 1.00H), 4.80-4.48 (m, 1.80H), 3.96 (br. s, 1.54H), 3.29-3.02 (m, 3.89H), 3.02-2.85 (m, 2.01H), 2.79-2.58 (m, 2.96H), 2.58-2.39 (m, 2.81H), 2.39-2.22 (m, 2.12H), 2.20-0.73 (br. m, 85.87H), 0.67 (s, 2.78H). UPLC/ELSD: RT=3.55 min. MS (ES): m/z=928.71 [M+H]⁺ for C₅₄H₉₃N₃O₅S₂.

### Step 2: N,N-bis(2-(3-aminocyclobutyl)ethyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

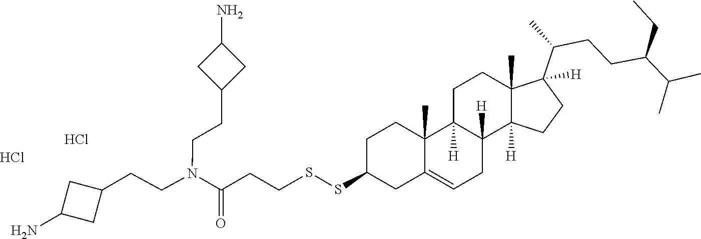

To a stirred solution of di-tert-butyl ((((3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)azanediyl)bis(ethane-2,1-diyl))bis(cyclobutane-3,1-diyl))dicarbamate (0.182 g, 0.196 mmol) in DCM (2.8 mL) cooled to 0° C. was added 4 N HCl in dioxane (0.49 mL) dropwise. The reaction mixture stirred at rt and was monitored by LCMS. At 19 h, the reaction mixture was diluted with MTBE to 20 mL, then centrifuged (10,000×g for 15 min at 4° C.). Supernatant was decanted, the solids were rinsed with MBTE, suspended in MTBE, then concentrated to afford N,N-bis(2-(3-aminocyclobutyl)ethyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride (0.129 g, 0.154 mmol, 78.7%) as a white solid. ¹H NMR (300 MHz, CD₃OD, reported as observed in spectrum): δ 5.45-5.30 (m, 1.00H), 3.98-3.73 (m, 0.80H), 3.73-3.48 (m, 1.70H), 3.02-2.87 (m, 2.18H), 2.85-2.72 (m, 2.11H), 2.72-0.78 (br. m, 64.25H), 0.78-0.68 (m, 2.99H). UPLC/ELSD: RT=2.24 min. MS (ES): m/z=386.14 [M+2Na]²⁺ for C₄₄H₇₉Cl₂N₃OS₂.

### DU. Compound SA188: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(2-(3-aminocyclobutyl)ethyl)amino)-5-oxopentanoate dihydrochloride

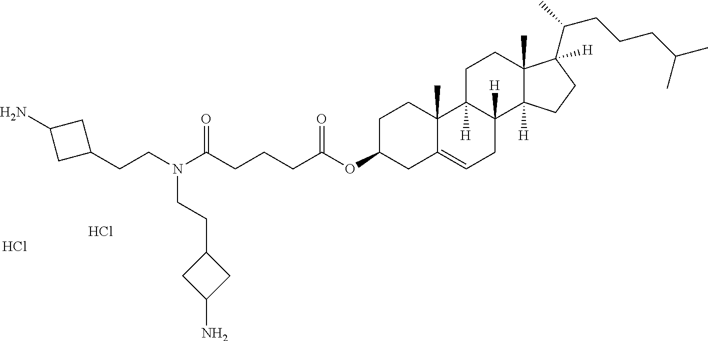

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(2-(3-((tert-butoxycarbonyl)amino)cyclobutyl)ethyl)amino)-5-oxopentanoate

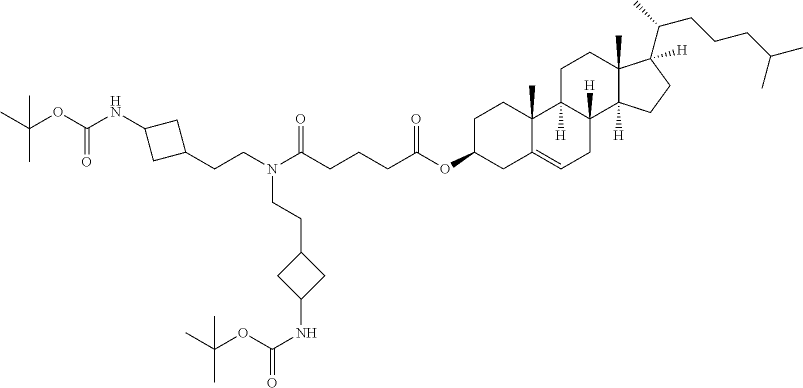

To a solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.100 g, 0.200 mmol), tert-butyl N-(3-{2-[(2-{3-[(tert-butoxycarbonyl)amino]cyclobutyl}ethyl)amino]ethyl}cyclobutyl)carbamate (0.082 g, 0.20 mmol), and DMAP (0.051 g, 0.42 mmol) in DCM (2 mL) was added EDC-HCl (0.077 g, 0.40 mmol). The reaction mixture stirred at rt and was monitored by LCMS. At 18 h, the reaction mixture was diluted with DCM (15 mL) and washed with 5% aq. NaHCO₃ soln. The aqueous was extracted with DCM (10 mL). The combined organics were passed through a hydrophobic frit, dried over Na₂SO₄, and concentrated. The crude material was purified via silica gel chromatography (35-70% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(2-(3-((tert-butoxycarbonyl)amino)cyclobutyl)ethyl)amino)-5-oxopentanoate (0.145 g, 0.162 mmol, 81.2%) as a clear oil and as a mixture of geometric isomers. ¹H NMR (300 MHz, CDCl₃, reported as observed in spectrum): δ 5.45-5.29 (m, 1.00H), 4.83-4.50 (m, 2.68H), 4.20 (br. s, 0.55H), 3.96 (br. s, 1.48H), 3.30-2.99 (m, 3.84H), 2.61-2.40 (m, 2.80H), 2.40-2.20 (m, 5.64H), 2.19-0.74 (br. m, 80.09H), 0.74-0.61 (m, 2.90H). UPLC/ELSD: RT=3.33 min. MS (ES): m/z=894.79 [M+H]⁺ for C₅₄H₉₁N₃O₇.

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(2-(3-aminocyclobutyl)ethyl)amino)-5-oxopentanoate dihydrochloride

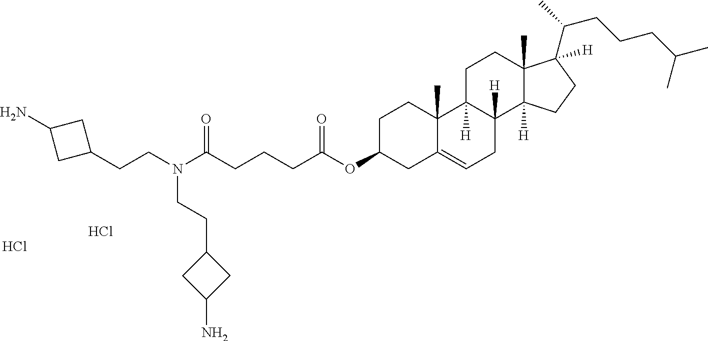

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(2-(3-((tert-butoxycarbonyl)amino)cyclobutyl)ethyl)amino)-5-oxopentanoate (0.138 g, 0.154 mmol) in DCM (2.1 mL) was added 4 N HCl in dioxane (0.37 mL). The reaction mixture stirred at rt and was monitored by LCMS. At 19 h, additional 4 N HCl in dioxane (0.10 mL) was added. At 69 h, the reaction mixture was diluted with MTBE to 20 mL, then centrifuged (10,000×g for 15 min at 4° C.). The supernatant was decanted, the solids were rinsed with MBTE, suspended in MTBE, then concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(2-(3-aminocyclobutyl)ethyl)amino)-5-oxopentanoate dihydrochloride (0.107 g, 0.138 mmol, 89.2%) as a white solid and as a mixture of geometric isomers. ¹H NMR (300 MHz, CD₃OD, reported as observed in spectrum): δ 5.44-5.25 (m, 1.06H), 4.60-4.45 (m, 1.00H), 3.93-3.73 (m, 0.72H), 3.73-3.48 (m, 1.70H), 3.28-3.18 (m, 3.35H), 2.61-0.79 (br. m, 60.78H), 0.77-0.63 (m, 3.00H). UPLC/ELSD: RT=1.99 min. MS (ES): m/z=347.93 [M+2H]²⁺ for C₄₄H₇₅N₃O₃.

### DV. Compound SA189: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis((3-aminobicyclo[1.1.1]pentan-1-yl)methyl)carbamate dihydrochloride

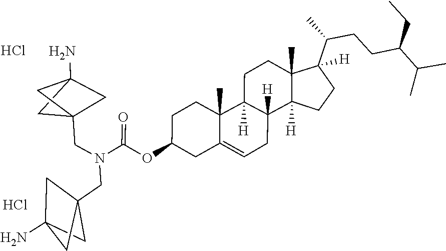

### Step 1: tert-butyl N-{3-[(2-nitrobenzenesulfonamido)methyl]bicyclo[1.1.1]pentan-1-yl}carbamate

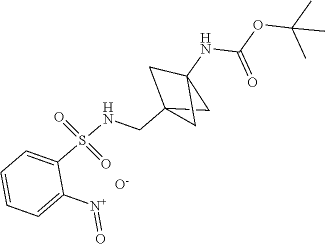

To a stirred solution of tert-butyl N-[3-(aminomethyl)bicyclo[1.1.1]pentan-1-yl]carbamate (0.980 g, 4.62 mmol) and triethylamine (0.80 mL, 5.7 mmol) in DCM (15 mL) cooled to 0° C. was added dropwise a solution of 2-nitrobenzenesulfonyl chloride (1.125 g, 5.078 mmol) in DCM (3 mL). After addition, the reaction mixture stirred at rt and was monitored by TLC. At 24 h, the reaction mixture was cooled to 0° C., then 5% aq. NaHCO₃ soln. (10 mL) was added. After warming to rt, the layers were separated. The aqueous was extracted with DCM (10 mL). The combined organics were passed through a hydrophobic frit, dried over Na₂SO₄, and concentrated. The crude material was purified via silica gel chromatography (20-60% EtOAc in hexanes) to afford tert-butyl N-{3-[(2-nitrobenzenesulfonamido)methyl]bicyclo[1.1.1]pentan-1-yl}carbamate (1.524 g, 3.834 mmol, 83.1%) as a yellow oil. UPLC/ELSD: RT=0.73 min. MS (ES): m/z=383.19 [(M+H)—(CH₃)₂C═CH₂+CH₃CN]⁺ for C₁₇H₂₃N₃O₆S. ¹H NMR (301 MHz, CDCl₃) δ 8.16-8.06 (m, 1H), 7.89-7.82 (m, 1H), 7.79-7.70 (m, 2H), 5.25 (t, J=6.0 Hz, 1H), 4.88 (br. s, 1H), 3.30 (d, J=5.9 Hz, 2H), 1.84 (s, 6H), 1.41 (s, 9H).

### Step 2: tert-butyl N-(3-{[N-({3-[(tert-butoxycarbonyl)amino]bicyclo[1.1.1]pentan-1-yl}methyl)-2-nitrobenzenesulfonamido]methyl}bicyclo[1.1.1]pentan-1-yl)carbamate

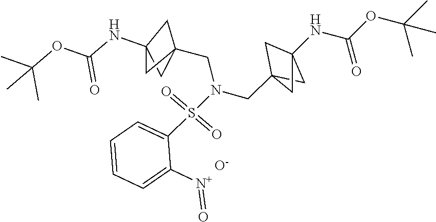

Tert-butyl N-{3-[(2-nitrobenzenesulfonamido)methyl]bicyclo[1.1.1]pentan-1-yl}carbamate (0.530 g, 1.33 mmol), tert-butyl N-[3-(bromomethyl)bicyclo[1.1.1]pentan-1-yl]carbamate (0.442 g, 1.60 mmol), potassium carbonate (0.369 g, 2.67 mmol), potassium iodide (0.111 g, 0.667 mmol), and propionitrile (8.0 mL) were combined in a sealed tube. The reaction mixture was heated at 150° C. with microwaves irradiation and was monitored by LCMS. At 4 h, the reaction mixture was filtered through a pad of celite rinsing with ACN. The filtrate was concentrated, then taken up in DCM (50 mL) and washed with 5% aq. NaHCO₃ soln. The aqueous was extracted with DCM (25 mL). The combined organics were passed through a hydrophobic frit, dried over Na₂SO₄, and concentrated. The crude material was purified via silica gel chromatography (30-60% EtOAc in hexanes) to afford tert-butyl N-(3-{[N-({3-[(tert-butoxycarbonyl)amino]bicyclo[1.1.1]pentan-1-yl}methyl)-2-nitrobenzenesulfonamido]methyl}bicyclo[1.1.1]pentan-1-yl)carbamate (0.549 g, 0.926 mmol, 69.5%) as a clear oil. UPLC/ELSD: RT=1.37 min. MS (ES): m/z=1185.75 (2M+H)⁺ for C₂₈H₄₀N₄O₈S. ¹H NMR (300 MHz, CDCl₃) δ 8.11-8.00 (m, 1H), 7.74-7.55 (m, 3H), 4.86 (br. s, 2H), 3.56 (s, 4H), 1.81 (s, 12H), 1.40 (s, 18H).

### Step 3: tert-butyl N-(3-{[({3-[(tert-butoxycarbonyl)amino]bicyclo[1.1.1]pentan-1-yl}methyl)amino]methyl}bicyclo[1.1.1]pentan-1-yl)carbamate

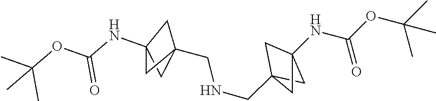 [SMILES: CC(C)(C)OC(=O)NC12CC(CNCC34CC(NC(=O)OC(C)(C)C)(C3)C4)(C1)C2]

To a mixture of tert-butyl N-(3-{[N—({3-[(tert-butoxycarbonyl)amino]bicyclo[1.1.1]pentan-1-yl}methyl)-2-nitrobenzenesulfonamido]methyl}bicyclo[1.1.1]pentan-1-yl)carbamate (0.529 g, 0.893 mmol) and potassium carbonate (0.370 g, 2.68 mmol) in DMF (8.0 mL) was added thiophenol (0.17 mL, 1.7 mmol). The reaction mixture stirred at rt and was monitored by LCMS. At 23 h, the reaction mixture was filtered rinsing with copious EtOAc. The filtrate was diluted with EtOAc to 150 mL, then washed with 5% aq. potassium carbonate (2×), water (3×), and brine. The organics were dried over Na₂SO₄ and concentrated. The crude material was purified via silica gel chromatography (0-20% (5% conc. aq. NH₄OH in MeOH) in DCM) to afford tert-butyl N-(3-{[({3-[(tert-butoxycarbonyl)amino]bicyclo[1.1.1]pentan-1-yl}methyl)amino]methyl}bicyclo[1.1.1]pentan-1-yl)carbamate (0.312 g, 0.766 mmol, 85.8%) as a white solid. UPLC/ELSD: RT=0.33 min. MS (ES): m/z=408.21 (M+H)⁺ for C₂₂H₃₇N₃O₄. ¹H NMR (300 MHz, CDCl₃) δ 4.92 (br. s, 2H), 2.76 (s, 4H), 1.90 (s, 12H), 1.44 (s, 18H), 0.67 (br. s, 1H).

### Step 4: di-tert-butyl (((((((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)carbonyl)azanediyl)bis(methylene))bis(bicyclo[1.1.1]pentane-3,1-diyl))dicarbamate

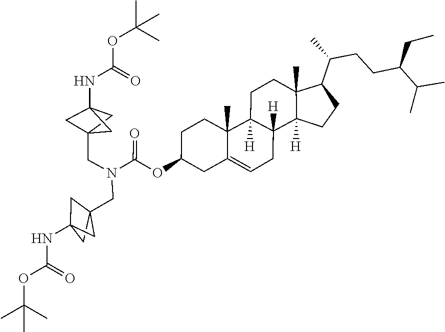

Sitosteryl 4-nitrophenyl carbonate (0.120 g, 0.207 mmol), tert-butyl N-(3-{[({3-[(tert-butoxycarbonyl)amino]bicyclo[1.1.1]pentan-1-yl}methyl)amino]methyl}bicyclo[1.1.1]pentan-1-yl)carbamate (0.101 g, 0.248 mmol), and triethylamine (0.09 mL, 0.65 mmol) were combined in PhMe (1.8 mL). The reaction mixture stirred at 90° C. and was monitored by LCMS. At 24 h, the reaction mixture stirred at 100° C. At 42 h, the reaction mixture was cooled to rt, diluted with DCM (10 mL), then washed with 5% aq. K₂CO₃ soln. (2×). The aqueous was extracted with DCM (10 mL). The combined organics were passed through a hydrophobic frit, dried over Na₂SO₄, and concentrated. The crude material was purified via silica gel chromatography (0-50% EtOAc in hexanes) to afford di-tert-butyl (((((((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)carbonyl)azanediyl)bis(methylene))bis(bicyclo[1.1.1]pentane-3,1-diyl))dicarbamate (0.137 g, 0.162 mmol, 78.0%) as a clear oil. UPLC/ELSD: RT=3.48 min. MS (ES): m/z=848.91 (M+H)⁺ for C₅₂H₈₅N₃O₆. ¹H NMR (300 MHz, CDCl₃) δ 5.43-5.28 (m, 1H), 4.89 (br. s, 2H), 4.54-4.37 (m, 1H), 3.45-3.22 (m, 4H), 2.40-2.17 (m, 2H), 2.08-1.75 (m, 17H), 1.73-0.76 (m, 49H), 1.01 (s, 3H), 0.92 (d, J=6.3 Hz, 3H), 0.68 (s, 3H).

### Step 5: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis((3-aminobicyclo[1.1.1]pentan-1-yl)methyl)carbamate dihydrochloride

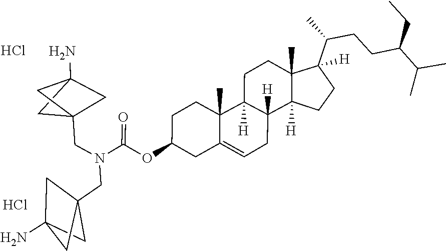

To a stirred solution of di-tert-butyl (((((((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)carbonyl)azanediyl)bis(methylene))bis(bicyclo[1.1.1]pentane-3,1-diyl))dicarbamate (0.131 g, 0.154 mmol) in DCM (2.0 mL) was added 4 N HCl in dioxane (0.27 mL, 1.1 mmol). The reaction mixture stirred at rt and was monitored by LCMS. At 22 h, the reaction mixture was diluted with MTBE to 20 mL, then centrifuged (10,000×g for 30 min at 4° C.). The supernatant was drawn off. The solids were rinsed sparingly with MTBE, suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis((3-aminobicyclo[1.1.1]pentan-1-yl)methyl)carbamate dihydrochloride (0.108 g, 0.133 mmol, 86.2%) as a white solid. UPLC/ELSD: RT=1.89 min. MS (ES): m/z=366.17 [(M+2H)+2CH₃CN]²⁺ for C₄₂H₆₉N₃O₂. ¹H NMR (300 MHz, MeOD) δ 5.44-5.34 (m, 1H), 4.50-4.33 (m, 1H), 3.55-3.36 (m, 4H), 2.41-2.27 (m, 2H), 2.14-0.77 (m, 48H), 1.06 (s, 3H), 0.96 (d, J=6.4 Hz, 3H), 0.73 (s, 3H).

### Example 7

### Protein Expression Data in Human Cervical Cancer Epithelial Cell (HeLa) Model

LNPs were prepared according to Example 1 using NPI-Luc as the mRNA construct. NPI-Luc is a dual read reporter made by adding a 5×V5 tag and a C-myc nuclear localization sequence at the N-terminus of Firefly Luciferase to enhance the signal to noise ratio. Protein expression can be detected using OneGLo assays with luminescence readout or by immunofluorescence with anti-V5 antibodies. Protein expression was evaluated according to the procedure outlined in Example 6. The LNPs are dosed in 4 wells and the average response was reported. For the HeLa assay the luminescence read (RLU) were normalized to cell counts. The results are shown in Table 9.

TABLE 9

| | | Average | Standard |
|---|---|---|---|
| | | protein | deviation |
| | | expression | protein |
| Sterol | | per cell | expression |
| Amine | LNP Core | (RLU per cell) | per cell |
| | | 0.4* | 0.0* |
| SA3 | Compound 18, DSPC, Cholesterol, | 111.0 | 11.1 |
| | and DMG-PEG 2K | | |
| SA4 | Compound 18, DSPC, Cholesterol, | 248.5 | 80.5 |
| | and Compound 428 | | |
| SA6 | Compound 18, DSPC, Cholesterol, | 196.2 | 30.3 |
| | and Compound 428 | | |
| SA10 | Compound 18, DSPC, Cholesterol, | 236.6 | 81.3 |
| | and Compound 428 | | |
| SA11 | Compound 18, DSPC, Cholesterol, | 5.3 | 2.9 |
| | and DMG-PEG 2K | | |
| SA16 | Compound 18, DSPC, Cholesterol, | 14.5 | 0.8 |
| | and Compound 428 | | |
| SA23 | Compound 18, DSPC, β-sitosterol, | 868.8 | 72.8 |
| | Cholesterol, and DMG-PEG 2K | | |
| SA24 | Compound 18, DSPC, Cholesterol, | 457.2 | 45.2 |
| | and DMG-PEG 2K | | |
| SA26 | Compound 18, DSPC, Cholesterol, | 84.3 | 35.3 |
| | and DMG-PEG 2K | | |
| SA29 | Compound 18, DSPC, β-sitosterol, | 550.3 | 91.6 |
| | Cholesterol, and DMG-PEG 2K | | |
| SA30 | Compound 18, DSPC, β-sitosterol, | 648.3 | 157.5 |
| | Cholesterol, and DMG-PEG 2K | | |
| SA31 | Compound 18, DSPC, β-sitosterol, | 643.6 | 69.5 |
| | Cholesterol, and DMG-PEG 2K | | |
| SA39 | Compound 18, DSPC, Cholesterol, | 201.9 | 6.0 |
| | and DMG-PEG 2K | | |
| SA32 | Compound 18, DSPC, Cholesterol, | 301.8 | 44.5 |
| | and DMG-PEG 2K | | |

*Data taken in phosphate buffered saline solution

### Example 8

### LNP Cellular Uptake and Protein Expression Data in Healthy HBE Cells

LNPs were prepared according to Example 1 using NPI-Luc as the mRNA construct. NP-Luc is a dual read reporter made by adding a 5×V5 tag and a C-myc nuclear localization sequence at the N-terminus of Firefly Luciferase to enhance the signal to noise ratio. Protein expression can be detected using OneGLo assays with luminescence readout or by immunofluorescence with anti-V antibodies. LNP cellular uptake and protein expression was evaluated according to the procedure outlined in Example 5. The results are shown in Table 10.

TABLE 10

| | | | Standard | | Standard |
|---|---|---|---|---|---|
| | | Average | deviation | Average | deviation |
| | | accumulation | accumulation | protein | protein |
| | | of LNP in | of LNP in | expression in | expression in |
| | | healthy HBE | healthy HBE | healthy HBE | healthy HBE |
| Sterol | | (% positive | (% positive | (% V5 positive | (% V5 positive |
| Amine | LNP Core | cells) | cells) | cells) | cells) |
| | | 1.6* | 0.2* | 0.1* | 0.0* |
| SA3 | Compound 18, | 43.7 | 1.9 | 9.4 | 1.2 |
| | DSPC, Cholesterol, | | | | |
| | and DMG-PEG 2K | | | | |
| SA23 | Compound 18, | 34.3 | 2.2 | 9.1 | 1.4 |
| | DSPC, β-sitosterol, | | | | |
| | Cholesterol, and | | | | |
| | DMG-PEG 2K | | | | |
| SA29 | Compound 18, | 56.2 | 3.7 | 12.1 | 3.5 |
| | DSPC, β-sitosterol, | | | | |
| | Cholesterol, and | | | | |
| | DMG-PEG 2K | | | | |
| SA30 | Compound 18, | 49.9 | 2.0 | 11.9 | 3.8 |
| | DSPC, β-sitosterol, | | | | |
| | Cholesterol, and | | | | |
| | DMG-PEG 2K | | | | |
| SA31 | Compound 18, | 43.2 | 3.8 | 11.1 | 2.5 |
| | DSPC, β-sitosterol, | | | | |
| | Cholesterol, and | | | | |
| | DMG-PEG 2K | | | | |

*Data taken in phosphate buffered saline solution

### Example 9

### Nanoparticle Zeta Potential

LNPs were prepared according to Example 1. Zeta potential was measured by diluting LNPs to [mRNA]0.01 mg/mL in 0.1×PBS on a Malvern Zetasizer (Nano ZS). The results are shown in Table 11.

TABLE 11

| | | Zeta |
|---|---|---|
| Sterol | | Potential |
| Amine | Core LNP | (mV) |
| SA3 | Compound 18, DSPC, Cholesterol, and DMG-PEG | 10.6 |
| | 2K | |
| SA23 | Compound 18, DSPC, β-sitosterol, Cholesterol, and | 12.7 |
| | DMG-PEG 2K | |
| SA29 | Compound 18, DSPC, β-sitosterol, Cholesterol, and | 8.3 |
| | DMG-PEG 2K | |
| SA30 | Compound 18, DSPC, β-sitosterol, Cholesterol, and | 8.7 |
| | DMG-PEG 2K | |
| SA31 | Compound 18, DSPC, β-sitosterol, Cholesterol, and | 8.6 |
| | DMG-PEG 2K | |

### Example 10

### In Vivo Studies

Dosing Procedure A: Intratracheal mRNA Delivery

Animals are anesthetized under isoflurane. The tongue is displaced and a small diameter cannula is inserted into the trachea (oropharyngeal route). The cannula tip is passed through the vocal chords, down the trachea so that the tip is very near, but not touching, the carina. Upon placement, 50 μL (mouse) or 200 μL (rat) of formulation is infused into the lungs. After 30 seconds upright, animads are released into a recovery cage and returned to their respective cages once recovered.

### Dosing Procedure B: Nose-Only Aerosol Exposure

Aerosol is generated using a vibrating mesh nebulizer and a defined inlet air flow rate. Aerosol is introduced into the rodent nose-only directed flow exposure chamber by first passing through a mixing chamber before flowing into the exposure tier. Animals are exposed to fresh aerosol at each nose port, which is then exhausted out of the system.

Animals were trained to the nose-only dosing cones for three days prior to initiation of the study. On study day, animals were placed into the dosing cones that were then attached to the aerosol exposure chamber for designated exposure times of 60, 120 or 240 minutes per group for lung doses of 0.4, 0.6 and 1.1 mpk. Animals were monitored continuously throughout the entire exposure and subsequently for any observable adverse reactions. Aerosol concentration (mRNA) and aerodynamic particle size distribution were monitored at the dosing port before and after each dosing occasion to evaluate achieved dose levels and respirable aerosol particle size targets (1-4 μm for rat) respectively.

### Sample Collection and Assays Procedure A: Tissue Collection for Histology

Trachea, lungs and for the aerosol study nasal cavities, nasopharynx and larynx are collected for analysis. Lungs are inflated with 10% NBF fixative and trachea tied off to maintain inflation. Lungs are removed en bloc with attached trachea, bronchi and lobes. Whole lungs en bloc are fixed in 10% NBF at room temperature for at least 24 hours with a maximum of 48 hours and then removed from fixative and placed in PBS. Samples are immediately sent to be processed for paraffin 5-micron sections and H&E staining.

For the aerosol study, nasal cavities, nasopharynx and larynx were also collected in addition to trachea and lungs.

### Sample Collection and Assays Procedure B: Immunohistochemistry (IHC)

IHC was performed on FFPE sections using the Leica Bond RX autostainer. NPI-Luc protein expression was detected by anti-V5 tag antibody at a 1:100 dilution. V5 antibody was detected with the Bond Polymer Refine Detection kit followed by hematoxylin and bluing reagent counterstain. Images were imaged at 20× magnification with the Panoramic 250 Flash III whole slide scanner. Image analysis was completed with Indica Labs HALO image analysis software. Trachea, lung and/or nasal cavity images were analyzed to capture total tracheal, bronchial or nasal epithelial cells and data expressed when appropriate as % V5 positive epithelial cells per total epithelial cells per animal.

LNP Protein Expression Data in Mouse after Single Dose of mRNA-LNP by Intratracheal Delivery

LNPs were prepared according to Example 1 using NPI-Luc as the mRNA construct. LNPs were delivered to mice by intratracheal instillation for a dose of ˜0.7 mpk. LNP protein expression in respiratory epithelium was evaluated according to sample collection and assay procedures A and B. The results are shown in Table 12. LNPs with cationic agent disposed primarily on the outer surface demonstrated positive respiratory epithelium protein expression in the trachea and bronchi.

TABLE 12

| | | | | Standard |
|---|---|---|---|---|
| | | | Average | deviation |
| | | | % V5 | % V5 |
| | | | positive | positive |
| Sterol | | | cells/ | cells/ |
| Amine | LNP Core | Tissue | total cells | total cells |
| | | Mouse | 0.09* | 0.04* |
| SA3 | Compound 18, DSPC, | Trachea | 40.7 | 0.39 |
| | Cholesterol, and DMG-PEG | | | |
| | 2K | | | |
| SA23 | Compound 18, DSPC, β- | | 0.8 | 0.66 |
| | sitosterol, Cholesterol, and | | | |
| | DMG-PEG 2K | | | |
| SA31 | Compound 18, DSPC, β- | | 27.07 | 8.96 |
| | sitosterol, Cholesterol, and | | | |
| | DMG-PEG 2K | | | |
| | | Mouse | 0.08* | 0.05* |
| SA3 | Compound 18, DSPC, | Bronchi | 9.33 | 4.93 |
| | Cholesterol, and DMG-PEG | (left | | |
| | 2K | lung) | | |
| SA23 | Compound 18, DSPC, β- | | 0.98 | 0.42 |
| | sitosterol, Cholesterol, and | | | |
| | DMG-PEG 2K | | | |
| SA31 | Compound 18, DSPC, β- | | 10.52 | 6.59 |
| | sitosterol, Cholesterol, and | | | |
| | DMG-PEG 2K | | | |

*Data taken in phosphate buffered saline solution

LNP Protein Expression Data in Rat after Single Dose of mRNA-LNP by Intratracheal Delivery

LNPs were prepared according to Example 1 using NPI-Luc as the mIRNA construct. LNPs were delivered to rats by intratracheal instillation for a dose of ˜1.2 mpk. LNP protein expression in respiratory epithelium was evaluated according to sample collection and assay procedures A and B. The results are shown in Table 13. LNPs with cationic agent disposed primarily on the outer surface demonstrated positive respiratory epithelium protein expression in the trachea and bronchi.

TABLE 13

| | | | Average | Standard |
|---|---|---|---|---|
| | | | % V5 | deviation |
| | | | positive | % V5 |
| | | | cells/ | positive |
| Sterol | | | total | cells/ |
| Amine | LNP Core | Tissue | cells | total cells |
| | | Rat | 0.03* | 0.03* |
| SA3 | Compound 18, DSPC, | Trachea | 2.97 | 2.14 |
| | Cholesterol, and DMG-PEG 2K | | | |
| SA23 | Compound 18, DSPC, β- | | 0.83 | 0.58 |
| | sitosterol, Cholesterol, and | | | |
| | DMG-PEG 2K | | | |
| | | Rat | 0.11* | 0.11* |
| SA3 | Compound 18, DSPC, | Bronchi | 4.95 | 4.38 |
| | Cholesterol, and DMG-PEG 2K | (left and | | |
| SA23 | Compound 18, DSPC, β- | right | 1.38 | 0.95 |
| | sitosterol, Cholesterol, and | lung) | | |
| | DMG-PEG 2K | | | |

*Data taken in phosphate buffered saline solution

LNP Protein Expression Data in Rat after Single Dose of mRNA-LNP by Aerosol Delivery

LNPs were prepared according to Example 7 using NPI-Luc as the mRNA construct. LNPs were delivered to rats by aerosol delivery using a nose-only aerosol dosing system. LNP protein expression in respiratory epithelium was evaluated according to sample collection and assay procedures A and B. The results are shown in Table 14. Respiratory epithelium in the nasal cavity, trachea and bronchi were positive for protein expression after aerosol delivery of LNPs.

TABLE 14

| | | | | Average | Standard |
|---|---|---|---|---|---|
| | | | | % V5 | deviation |
| | | | | positive | % V5 |
| | | | | cells/ | positive |
| Sterol | | | Dose | total | cells/ |
| Amine | LNP Core | Tissue | (mpk) | cells | total cells |
| | | Rat | | 0.20* | 0.19* |
| SA3 | Compound 18, DSPC, | Trachea | 0.4 | 0.29 | 0.16 |
| | Cholesterol, and | | 0.6 | 0.16 | 0.15 |
| | DMG-PEG 2K | | 1.1 | 0.65 | 0.39 |
| | | Rat | | 0.03* | 0.02* |
| SA3 | Compound 18, DSPC, | Bronchi | 0.4 | 0.13 | 0.07 |
| | Cholesterol, and | (left and | 0.6 | 0.13 | 0.03 |
| | DMG-PEG 2K | right | 1.1 | 0.46 | 0.17 |
| | | lung) | | | |
| | | Rat | | 0.18* | 0.16* |
| SA3 | Compound 18, DSPC, | Nasal | 0.4 | 0.64 | 0.76 |
| | Cholesterol, and | Cavity | 0.6 | 1.20 | 1.08 |
| | DMG-PEG 2K | | 1.1 | 2.84 | 3.05 |

*Data taken in Tris based buffered solution

### Example 11

### In Vivo Studies—Localization of Drug Product after Intranasal Administration

Mice were anesthetized under isoflurane. The tongue was displaced, and a small diameter cannula was inserted into the trachea (oropharyngeal route). The cannula tip was passed through the vocal chords, down the trachea so that the tip is very near, but not touching, the carina. Upon placement, 50 μL (mouse) of formulation was infused into the lungs. After 30 seconds upright, animals were released into a recovery cage and returned to their respective cages once recovered. Two vaccine dose levels, 20 μg and 5 μg, were tested at two different volumes, 10 μL per nostril and 25 μL per nostril. PBS at the two different volumes was used as a control.

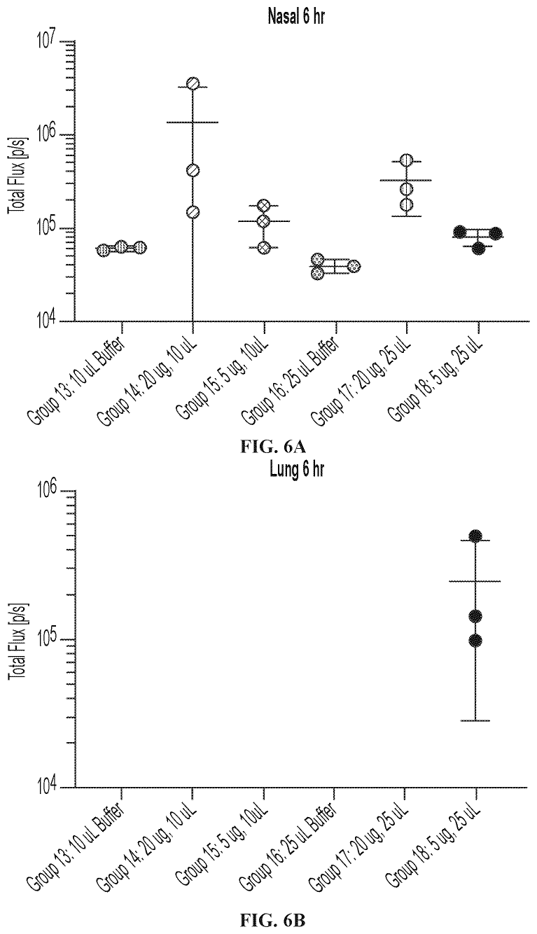

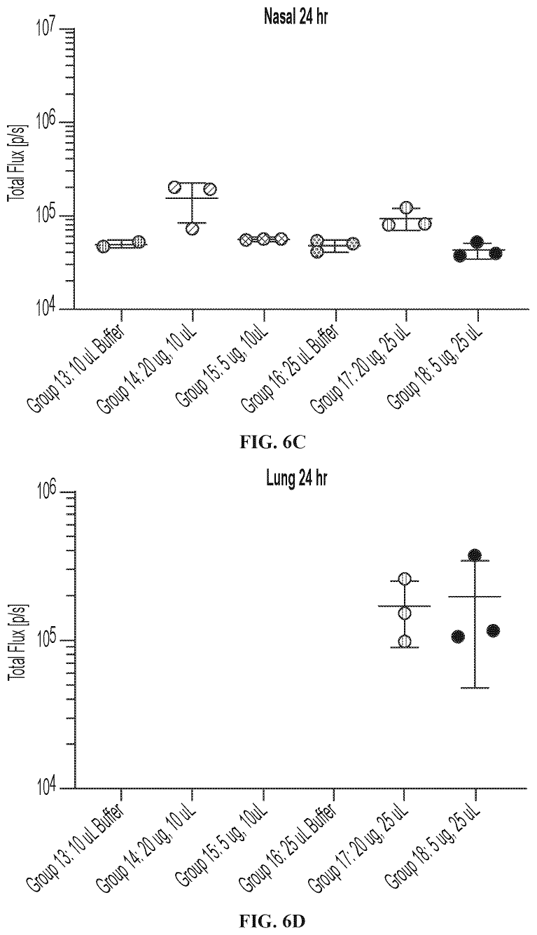

Six and 24 hours after dosing, whole body IVIS imaging, focusing on the nasal cavity and lungs was performed on the mice. The results are shown in FIGS. 6A-6D. At the six hour time point, the 20 μg doses were found to lead expression in the nasal cavity (FIG. 6A), while only one mouse in the 5 μg/25 μL group showed any luciferase expression in the lung (FIG. 6B). At 24 hours, the 20 μg doses were found to lead expression levels in the nasal cavity; however, there was decreased expression relative to the 6 hour time point (FIG. 6C). With respect to luciferase expression in the lung at 24 hours, expression was only observed in the two 25 μL groups, and it was decreased relative to the level observed at six hours (FIG. 6D).

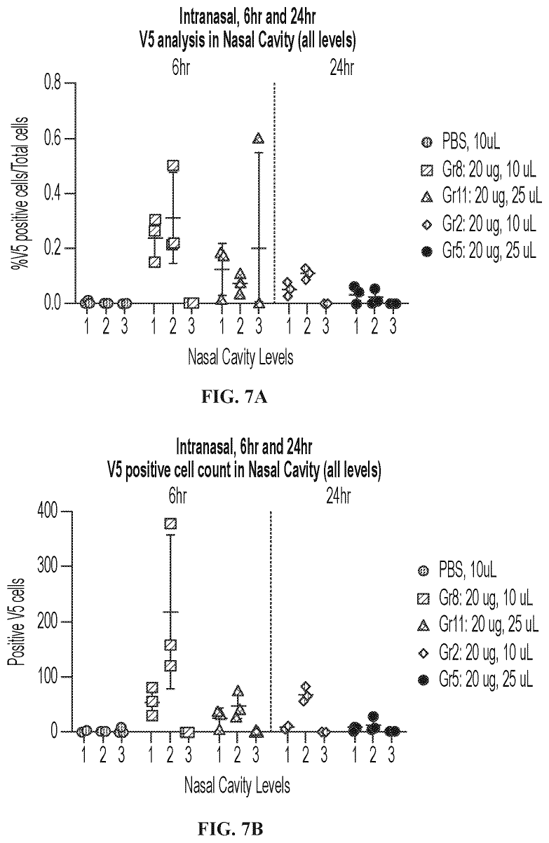

Six and 24 hours after dosing, immunohistochemistry (IHC) was performed to analyze histological sections the respiratory system, including the trachea, lungs, and nasal-associated lymphoid tissue (NALT). The results from the nasal cavity at both time points are shown in FIGS. 7A-7B. Overall, low levels of mRNA and protein expression were observed at both time points at volumes of 10 μL per nostril and 25 μL per nostril at 20 μg doses. The IHC lung and nasal cavity data was consistent with the imaging results. In particular, protein expression was only observed in a few animals from the 20 μg dose groups and was localized to inflammatory regions in the lung. In the trachea, some positive tracheal epithelial cells were observed. With respect to the nasal cavities, slightly higher luciferase protein expression was observed in the nasal cavities from the 10 VL20 μg dose groups as compared to the 25 μL/20 μg dose groups at both time points. Levels of mRNA were very low for both of the 20 μg dose groups.

### Example 12

### Immunogenicity and Efficacy of an mRNA Vaccine Encoding a Protein Antigen (AG1) Delivered Intranasally in Syrian Golden Hamster Model

The immunogenicity of an mRNA vaccine encoding a protein antigen, AG1, delivered intranasally in a Syrian golden hamster model was examined. Briefly, Syrian golden hamsters were administered a prime dose of the vaccine on day 1 and a boost on day 22 via intranasal (IN) or intramuscular (IM) routes. Serum was collected on days 21 and 42. On day 43, the hamsters were challenged with a virus comprising AG1 at 10⁵ PFU/100 μL, and samples were collected 3 and 14 days after the challenge. The experimental groups are shown in the table below:

TABLE 15

| | | | | Route | | |
|---|---|---|---|---|---|---|
| | | | | (20 | Day 1 | Day 22 |
| Group | N | Vaccine | LNP Formulation | μL/nare) | Immunization | Immunization |
| 1 | 10 | No vaccine | — | IN | PBS | PBS |
| 2 | 10 | mRNA | Compound 18/DMG | IN | 25 μg | 25 μg |
| 3 | 10 | encoding | Compound 18/DMG | IN | 5 μg | 5 μg |
| 4 | 10 | AG1 | Compound 18/DMG PA SA3 | IN | 25 μg | 25 μg |
| 5 | 10 | | Compound 18/DMG PA SA3 | IN | 5 μg | 5 μg |
| 6 | 10 | | Compound 18/SA23/DMG | IN | 25 μg | 25 ug |
| 7 | 10 | | Compound 18/SA23/DMG | IN | 5 μg | 5 μg |
| 8 | 10 | | Compound 25/DMG | IM (50 μL) | 1 μg | 1 μg |
| 9 | 10 | | | IM (50 μL) | 0.4 μg | 0.4 μg |

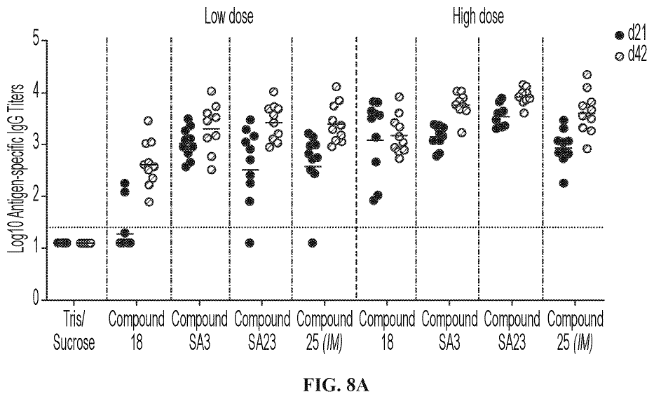

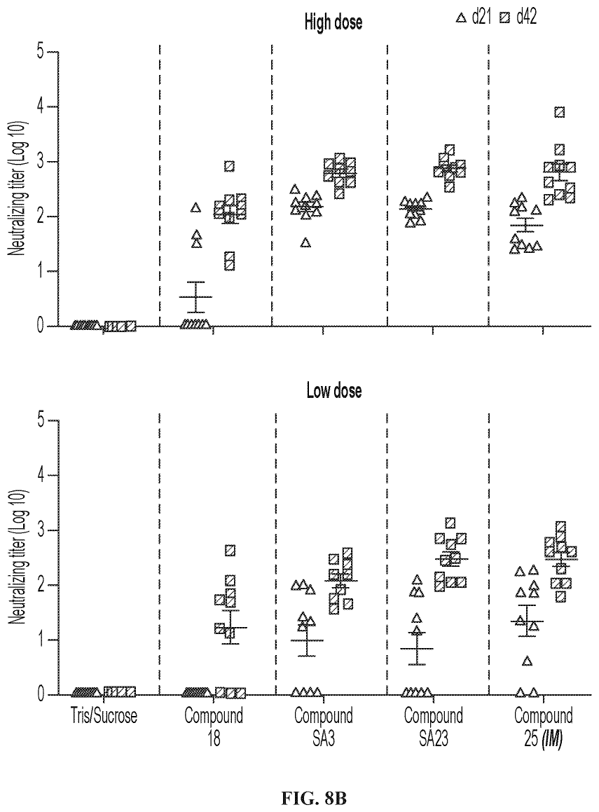

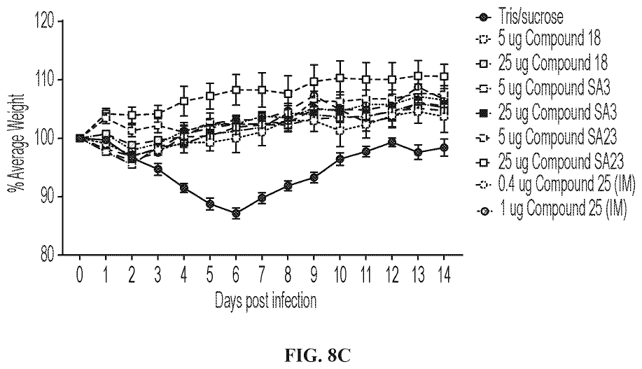

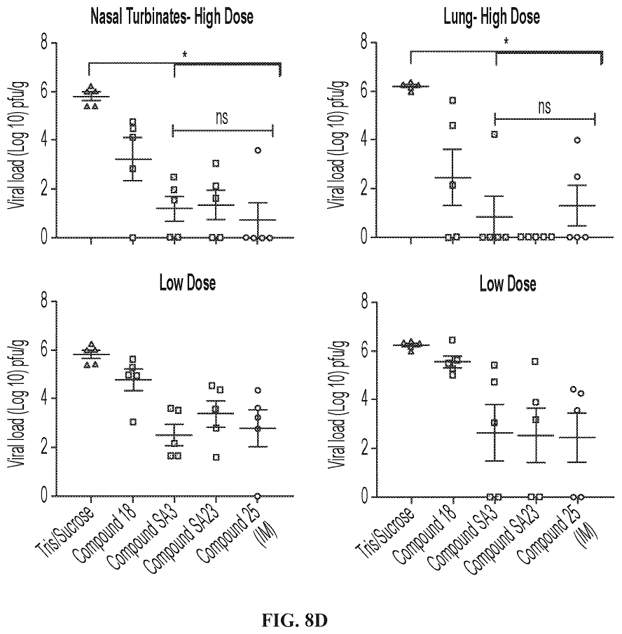

The results are shown in FIGS. 8A-8D. In particular, intranasal administration of the LNPs was found to induce specific binding titers to AG1 systemically. An intranasal boost increased the binding titers (FIG. 8A). Similarly, with respect to neutralizing titers, it was found that intranasal administration of the LNPs induces neutralizing titers systemically, and that the intranasal boost dose increases neutralizing titers (FIG. 8B). In addition, the two respiratory LNPs were found to outperform the control LNPs with respect to intranasal administration and were comparable to the intramuscular dose tested (FIG. 8B). After challenge, the immunized mice had minimal weight loss in comparison to a tris/sucrose buffer control group (FIG. 8C). With respect to viral load, respiratory LNPs were found to have decreased viral load in nasal turbinates and lungs compared to saline control three days after the viral challenge (FIG. 8D).

In a further experiment, the durability and efficacy of the vaccine is examined. The same protocol described above is used to administer two doses of intranasal vaccines comprising respiratory LNPs and mRNA encoding an AG1 protein. Following administration of the second dose on day 22, serum samples are taken monthly for six months. On day 168, the mice are challenged with a virus comprising AG1, 10⁵ PFU/100 μl challenge. Serum, nasal wash, and bronchoalveolar lavage samples are taken on day 168. Three days after the challenge, further samples are collected.

### Example 13

### Immunogenicity and Expression of a Seasonal mRNA Vaccine Encoding an Antigenic Protein of a Seasonal Virus Delivered Intranasally

The immunogenicity and expression of an mRNA vaccine encoding an mRNA vaccine comprising an ORF encoding AG2 delivered intranasally in respiratory LNPs in BALB/c mice was examined. Briefly, BALB/c mice were administered a prime dose of the vaccine on day 1 (low dose, 5 μg or high dose, 20 μg) and a boost on day 22 (same dose as received on day 1) intranasally. An alternative LNP formulation was used to deliver the vaccine intramuscularly (1 μg). Twenty-four hours after the first dose, in vivo expression in spleen, draining lymph nodes, lungs, and nasal tissue was measured. Serum was collected on days 21 and 43. Adaptive immune responses were measured at day 29 and 43 in spleen, draining lymph node, lungs, and nasal tissue.

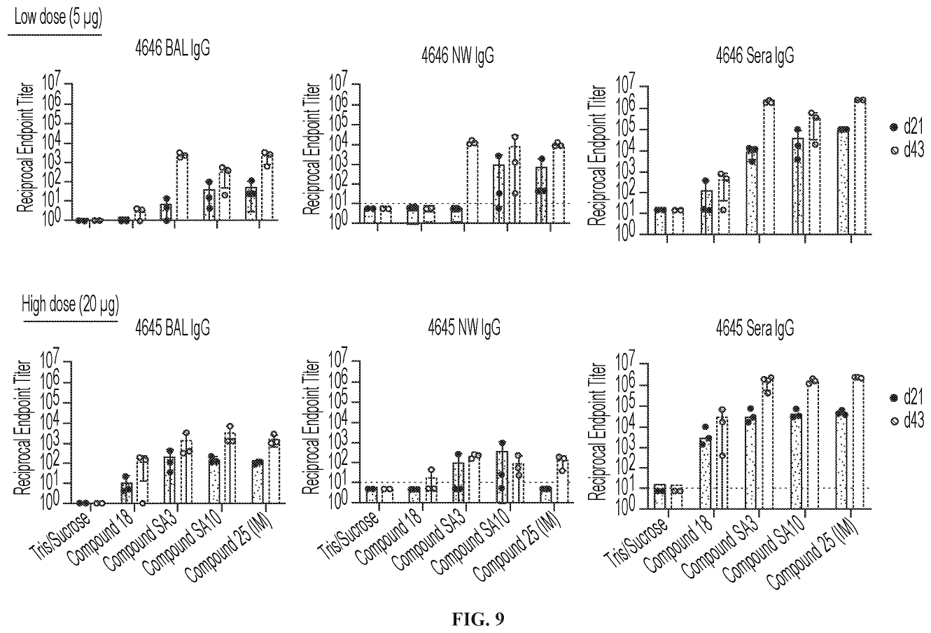

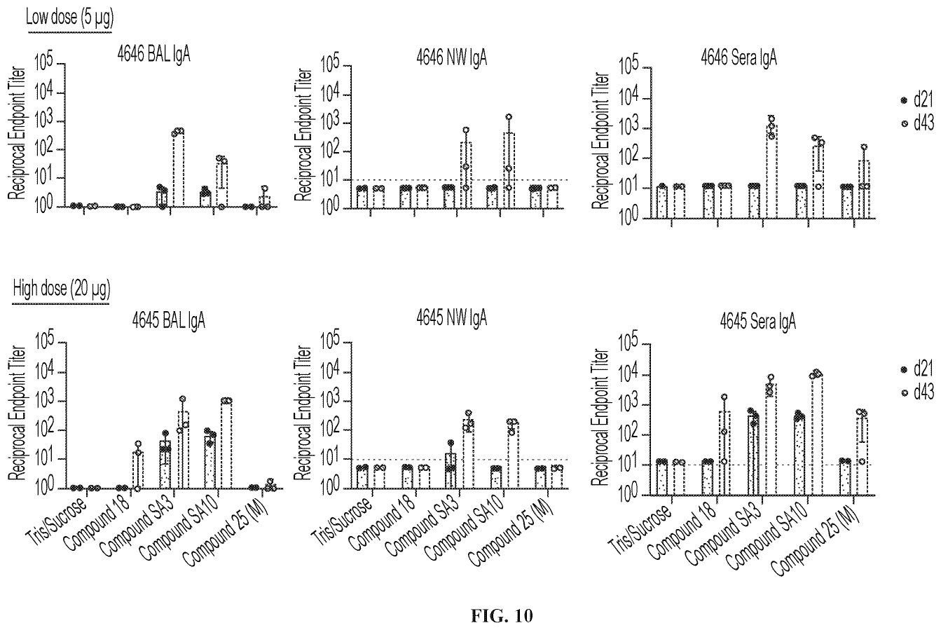

The results are shown in FIGS. 9 (IgG binding titers) and 10 (IgA binding titers), and demonstrate that the binding titers increased between doses and that the respiratory LNPs demonstrated titers that were comparable to the IM administration in both mucosal and systemic compartments (bronchoalveolar lavage, nasal wash, and serum) at both low and high doses.

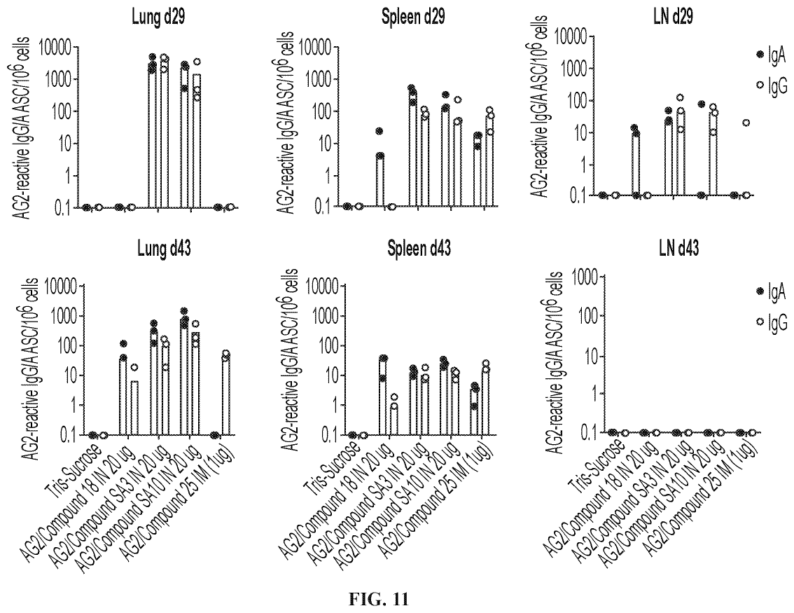

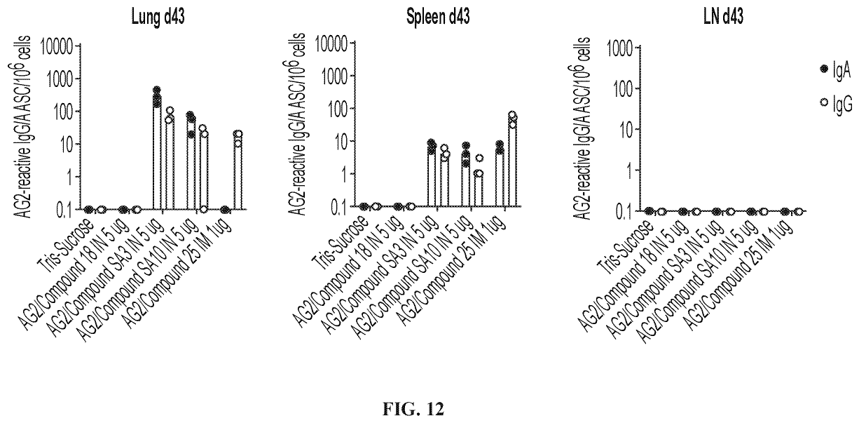

A B-cell ELISpot assay was performed. Briefly, plates were coated with antigen and a cell suspension from tissue was added. The plates were incubated overnight. Then, the cells were washed off, and the remaining bound antibody was detected with an antigen-specific antibody. The results are shown in FIGS. 11 and 12. Intranasal immunization was found to lead to both antigen-specific IgG and IgA secreting B cells in the mucosal components (lung, spleen, and lymph node) at the high dose (FIG. 11) and low dose (FIG. 12).

Neutralization was also measured with an assay. The results are shown in FIG. 13, and demonstrate that neutralization was present in both the high and low dose groups. The boost dose increased neutralization titers in all groups, except for the group formulated with Compound 18.

### Example 14

### Expression and Immunogenicity of an mRNA Vaccine Encoding an Antigenic Protein Delivered Intranasally in Mice

The immunogenicity and expression of an mRNA vaccine encoding an AG1 protein delivered intranasally in respiratory LNPs in BALB/c mice was examined. Briefly, BALB/c mice were administered a prime dose of the vaccine on day 1 (1 μg, 5 μg, or 20 μg) and a boost on day 22 (same dose as received on day 1) intranasally. An alternative LNP formulation was used to deliver the vaccine intramuscularly (1 μg). Twenty-four hours after the first dose, in vivo expression in spleen, draining lymph nodes, lungs, and nasal tissue was measured. Serum, bronchoalveolar lavage and nasal wash were collected on days 21 and 43. Adaptive immune responses were measured at day 29 and 43 in spleen, draining lymph node, lungs, and nasal tissue.

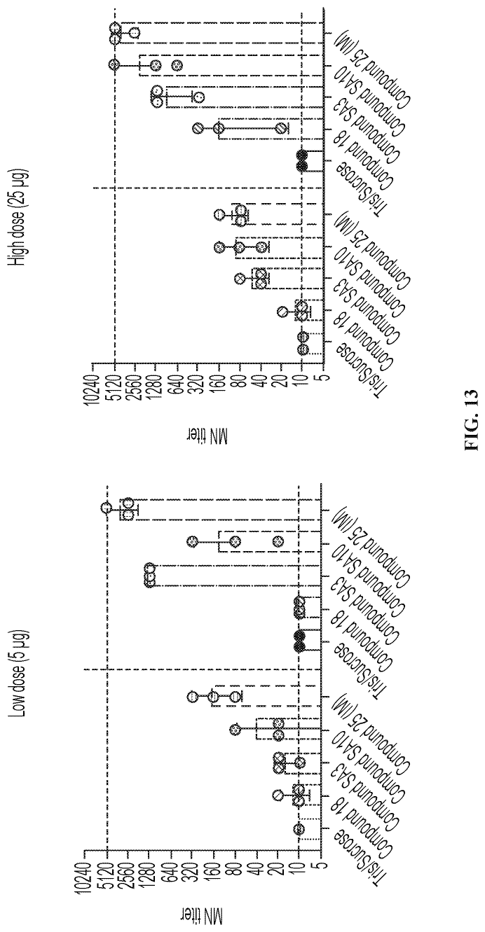

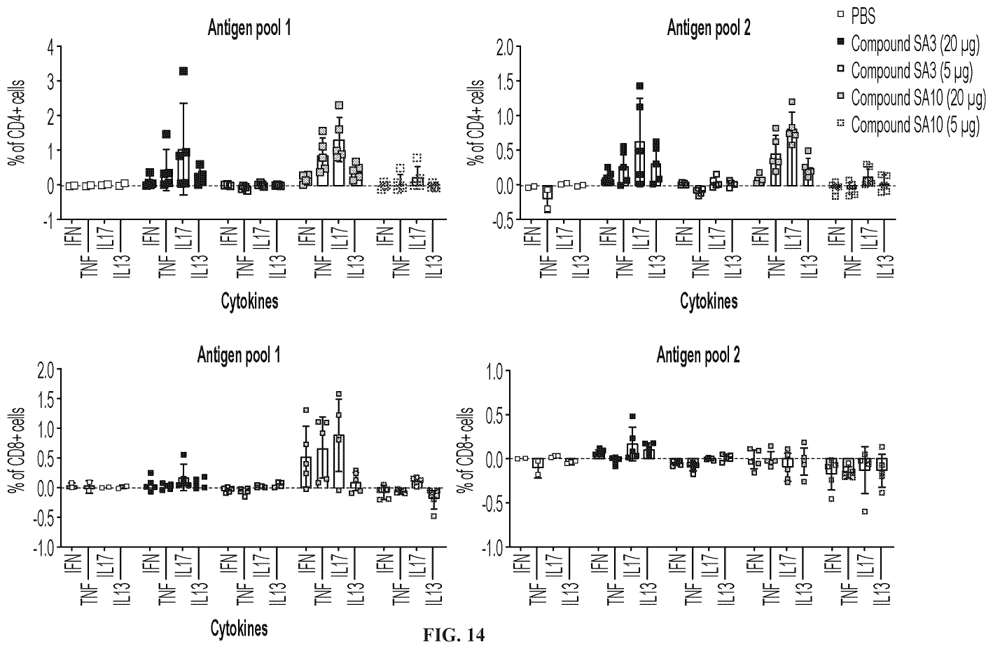

Antigen-specific T cell responses were measured, as shown in FIG. 14. The intranasal administration of mice with mRNA encoding an antigenic protein was found to induce both CD4+ and CD8+ effector responses, which was generally found to be increased more at the higher (20 μg) dose.

### Example 15

### Protection Study: mRNA Vaccine Encoding a Seasonal Viral Antigenic Protein Delivered Intranasally in Ferrets

The level of protection from an mRNA vaccine comprising an ORF encoding AG2 formulated in respiratory LNPs and delivered intranasally is examined in ferrets. Briefly, ferrets are administered the vaccine (75 μg delivered as 150 μL/nare) on days 1 and 22. On day 43, the ferrets are challenged with two different strains of the seasonal virus comprising AG2. Prior to each vaccination and the challenge, the serostatus of the ferrets is determined. Following challenge, nasal and throat swab samples are obtained daily and virus titers are measured. On day 46, pathology is performed.

### Example 16

### Immunogenicity and Efficacy of an mRNA Vaccine Encoding an Antigenic Protein Delivered Intranasally in Non-Human Primates

The immunogenicity and efficacy of an mRNA vaccine encoding an antigenic protein delivered intranasally to non-human primates (NHPs) is examined. Two different types of intransal adminstration are tested: droplet intranasal administration and administration with a device (MAD Nasal). Briefly, NHPs administered a prime dose of the vaccine on day 1 and a boost on day 29 via intranasal (IN) or intramuscular (IM) routes. On day 57, the NHPs are challenged with a virus comprising AG1. Serum, bronchoalveolar lavage and nasal wash are collected five days before the first administration of the vaccine, and on days 3, 6, 9, and 12 following the challenge. Serum samples are also collected on days 1, 15, 29, 43, and 52. Bronchoalveolar lavage and nasal wash samples are also collected on days 22 and 45. On day 36, peripheral blood mononuclear cells (PBMCs) are collected. Lung tissue samples are taken on days 13-16 following challenge.

### Example 17

### Heterologous Routes of Administration in Mice

To examine heterologous routes of administration, the following protocols are tested. Briefly, BALB/c mice (12/group) are administered a first dose of an mRNA vaccine comprising an ORF encoding AG2 and formulated in lipid nanoparticles is administered intranasally (IN) or intramuscularly (IM) on day 1 and then intranasally or intramuscularly on day 22, such that each of the combinations are tested: IN/IN, IN/IM, IM/IN, and IM/IM. Serum, bronchoalveolar lavage, and nasal wash are collected on days 21 and 43, and the adaptive immune response is measured on days 29 and 43 (with spleen, lung, and nasal tissue).

### Example 18

### Expression of COV2-2072 mAB in Nose and Lungs Following Intranasal Versus Intravenous Administration

The expression of an mRNA vaccine encoding a COV2-2072 protein (SEQ ID NOs: 20 and 23; Table 36) delivered intranasally in respiratory LNPs in BALB/c mice was examined. Briefly, BALB/c mice were administered a prime dose of the vaccine on day 1 (20 μg) intranasally. An alternative LNP formulation was used to deliver the vaccine intravenously (20 μg). At twenty-four, forty-eight, and ninety-six hours after the first dose, in vivo expression in lung, serum, nasal wash, and bronchoalveolar lavage was measured. The experimental groups are shown in the following table:

TABLE 16

| | | | LNP | | Volume/ | Dose |
|---|---|---|---|---|---|---|
| Group | N | Vaccine | Formulation | Route | nare (μL) | (μg) |
| 1 | 2 | No Vaccine | — | IN | 25 | — |
| 2 | 5 | COV2- | Compound 18/ | IV | 100 | 20 |
| | | 2072 | DMG | | | |
| 3 | 5 | | Compound SA3 | IN | 10 | 20 |
| 4 | 5 | | Compound SA3 | IN | 25 | 20 |
| 5 | 5 | | Compound SA10 | IN | 10 | 20 |
| 6 | 5 | | Compound SA10 | IN | 25 | 20 |

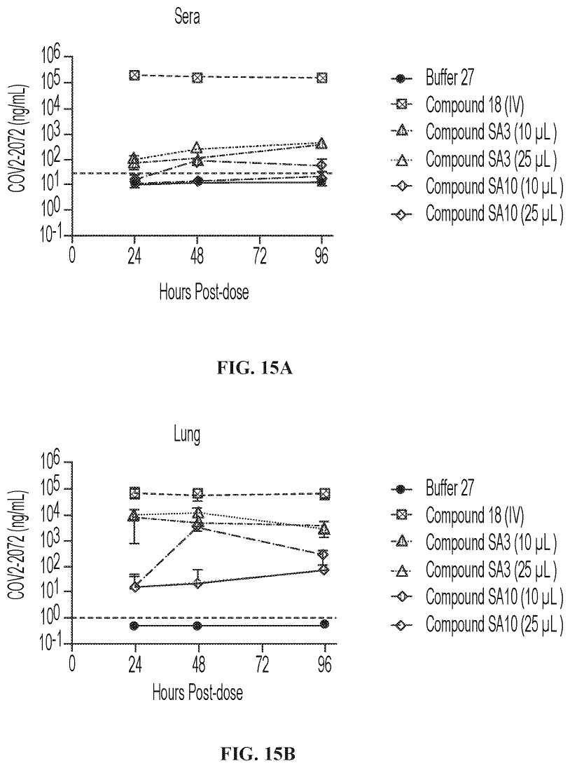

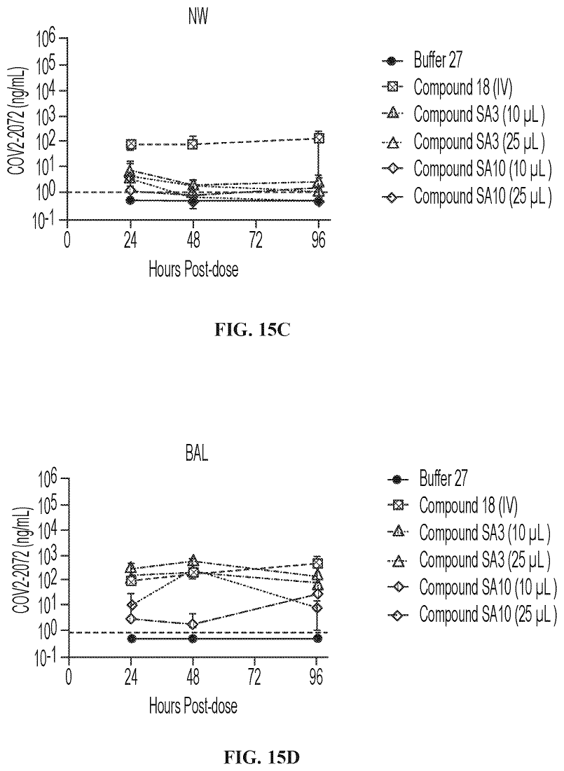

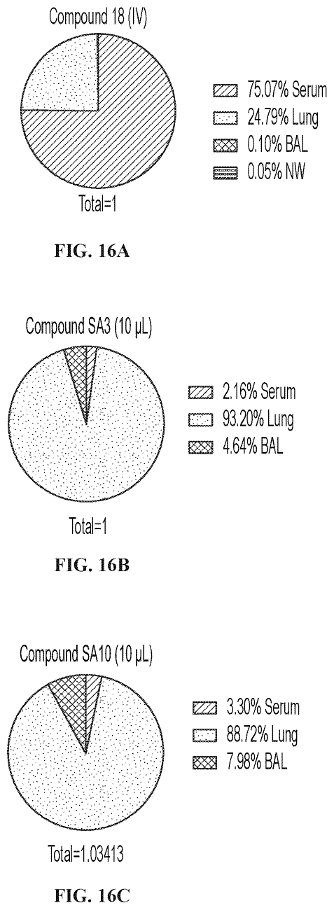

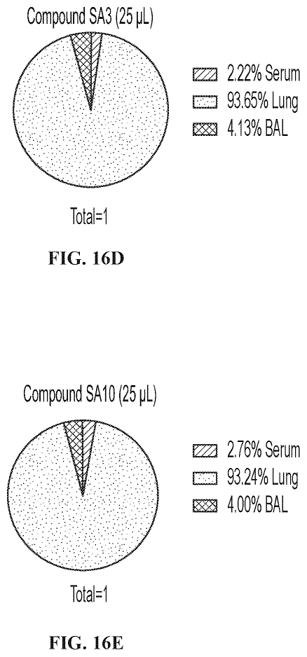

The results are shown in FIGS. 15A-15D and 16A-16E and demonstrate that the respiratory LNPs had detectable protein levels in sera (FIG. 15A), lung (FIG. 15B), nasal wash (FIG. 15C), and bronchoalveolar lavage (FIG. 15D). Protein expression levels in lung and bronchoalveolar lavage for mRNA vaccines encapsulated in Compound SA3 were similar to the mRNA vaccine administered intravenously. In addition, the antibody ratios revealed preferentially targeting of different compartments according to the route of administration. After intravenous administration, 75.07% of COV2-2072 antibodies in a sample originated from sera (FIG. 16A). In contrast, after intranasal administration with any LNP formulation, upwards of 88.72% of COV2-2072 antibodies in a sample originated from lung tissue (FIGS. 16B-16E).

### Example 19

### In Vivo Imaging to Screen Alternative Routes of Mucosal Immunization

To screen for alternative routes of mucosal immunization, mRNA-LNPs were generated by incorporating a codon-optimized firefly luciferase into a LNP formulation. CD-1 mice were intranasally administered a 20 μg dose of the mRNA-LNP on day 1. Whole body IVIS imaging was performed on the dorsal and ventral side at 6 hours and 18 hours post-dose. The experimental groups are shown in the following table:

TABLE 17

| Group | N | Vaccine | LNP Formulation |
|---|---|---|---|
| 1 | 4 | ffLuc | — |
| 2 | 4 | | Compound SA3 |
| 3 | 4 | | Compound SA10 |

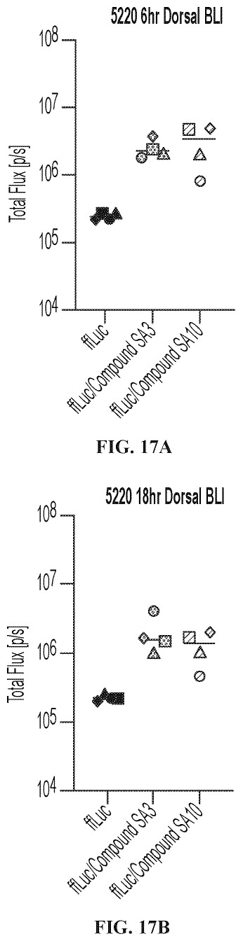

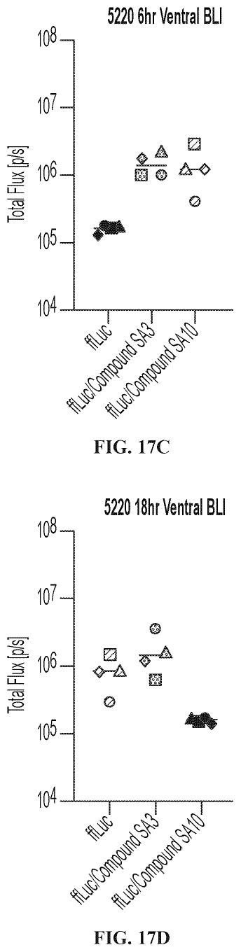

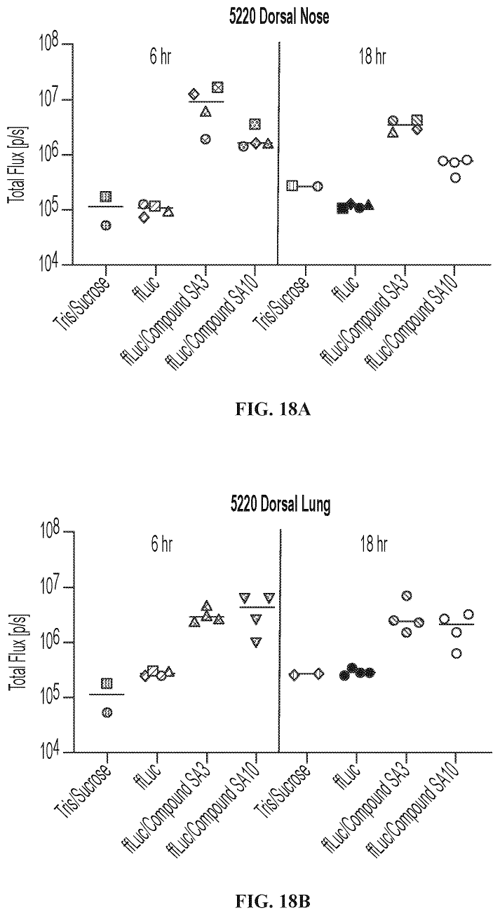

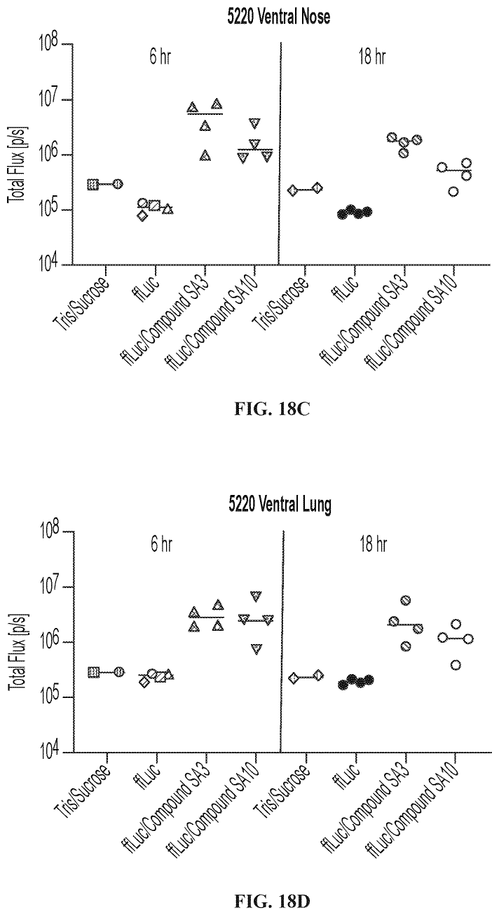

The results are shown in FIGS. 17A-17D and 18A-18D. Intranasal administration of mRNA-LNPs resulted in elevated expression in the nasal and lung cavity at 6 and 18 hours post-administration (FIG. 17A-17D). Intranasal administration of naked mRNA did not result in significant expression in the nose and lung relative to the control (FIG. 18A-18D).

### Example 20

### Vaccine Protection Study in Ferrets

The level of protection from an mRNA vaccine comprising an ORF encoding A/Victoria/2570/2019 (H₁N₁) (SEQ ID NO: 26; Table 36) formulated in LNPs and delivered intranasally is examined in ferrets. Briefly, ferrets are administered the vaccine on days 21 and 22. On day 43, the ferrets are challenged with two different strains of the virus. Prior to each vaccination and the challenge, the serostatus of the ferrets is determined. Following challenge, nasal washes and throat swab samples are obtained daily and virus titers are measured. On day 47, pathology is performed.

The experimental groups are shown in the following table:

TABLE 18

| | | | LNP | | Dose | Dose | Challenge (IN) |
|---|---|---|---|---|---|---|---|
| Group | N | mRNA Vaccine | Formulation | Route | Volume | (μg) | (250 μL/nare; 10⁴ pfu/mL) |
| 1 | 6 | A/Victoria/2570/2019 | pLNP #2 | IN | 150 | 75 | A/Victoria/2570/2019 |
| 2 | 6 | (H1N1) | | | μL/nare | 25 | (H1N1) |
| 3 | 6 | | Lipid H | IM | 500 μL | 75 | |
| 4 | 6 | | | | | 25 | |
| 5 | 6 | Mock (control mRNA) | pLNP #2 | IN | 150 | 75 | |
| 6 | 3 | | | | μL/nare | 75 | — |
| 7 | 6 | A/Victoria/2570/2019 | pLNP #2 | IN | 150 | 75 | A/Netherlands/602/2009 |
| 8 | 6 | (H1N1) | | | μL/nare | 25 | (H1N1) |
| 9 | 6 | | Lipid H | IM | 500 uL | 75 | |
| 10 | 6 | | | | | 25 | |
| 11 | 6 | Mock (control mRNA) | pLNP #2 | IN | 150 | 75 | |
| 12 | 3 | | | | μL/nare | 75 | — |

### Example 21

### Dose Curve for Intranasal Administration

The immunogenicity and expression of an mRNA vaccine comprising an ORF encoding HexaPro (ORF, SEQ ID NO: 3; HexaPro protein, SEQ ID NO: 5; Table 36) delivered intranasally in respiratory LNPs in mice is examined. Briefly, the mouse is administered a prime dose of the vaccine (10 μL/nare) on day 1 and a boost on day 22 (same dose as received on day 1) intranasally. Bronchoalveolar lavage, nasal wash, and sera samples are collected on day 21 and day 36. Lung, spleen, and nasal polyp tissues are additionally collected on day 36. IgG and IgA binding titers are evaluated on day 21 and day 36. T Cell ELISpot, B Cell ELISpot, and IPT are additionally evaluated on day 36. The experimental groups are shown in Table 19.

TABLE 19

| Group | N | Vaccine | Dose | LNP Formulation |
|---|---|---|---|---|
| 1 | 6 | Tris/Sucrose | Buffer 27 | — |
| 2 | 10 | HexaPro | 100 μg | Compound SA3 |
| 3 | 10 | HexaPro | 80 μg | Compound SA3 |
| 4 | 10 | HexaPro | 40 μg | Compound SA3 |
| 5 | 10 | HexaPro | 20 μg | Compound SA3 |
| 6 | 10 | HexaPro | 10 μg | Compound SA3 |
| 7 | 10 | HexaPro | 5 μg | Compound SA3 |

### Example 22

### Intranasal Immunization of Mice for an Intravaginal Immune Response in HSV-Vaccinated Mice

To examine heterologous routes of administration, the following protocols were tested. Briefly, C57BL/6 mice were administered a first dose of an mRNA vaccine against HSV-2 and formulated in lipid particles. The sequences of the mRNAs encoding the HSV proteins are provided in Table 36. The vaccine was administered intranasally (IN) or intramuscularly (IM) on day 1 (prime) and then intranasally or intramuscularly on day 22 (boost), such that each of the combinations were tested: IN₁/IN₁, IN₁/IN₂, IM/IN, and IM/IM, where IN₁ and IN₂ are different intranasal vaccine formulations. All IM injections were at 1 μg in 50 μL using Compound 25. Bronchoalveolar lavage, nasal wash, sera, and female reproductive tract (FRT) samples were collected on days 21 and 36. Lung and spleen tissue are additionally collected on day 36. IgA and IgG binding titers were evaluated on day 21 and on day 36. Additional assays, including neutralization assay, antibody-dependent cellular cytotoxicity (ADCC), T Cell EliSpot, B Cell EliSpot, and histology of the female reproductive tract, were performed on day 36. The experimental groups are shown in the following table:

TABLE 20

| | | | | LNP | |
|---|---|---|---|---|---|
| Group | N | Vaccine | Dose | Formulation | Route |
| 1 | 6 | Buffer 27 | Buffer | — | IM/IN |
| | | | 27 | | (10 μL |
| | | | | | per nare) |
| 2 | 10 | gB(delta), | 40 μg | Compound | IN/IN |
| | | gC, | | SA3 (IN) | (10 μL |
| | | gD (2:1:1) | | | per nare) |
| 3 | 10 | gB(delta), | 40 μg | Compound | IM/IN |
| | | gC, | | 25 (IM) | (10 μL |
| | | gD (2:1:1) | | Compound | per nare) |
| | | | | SA3 (IN) | |
| 4 | 10 | gB(delta), | 40 μg | Compound | IN/IN |
| | | gC, | | SA3 (IN) | (10 μL |
| | | gD (2:1:1) | | Compound | per nare) |
| | | | | 25 (IM) | |
| 5 | 10 | gB(delta), | 1 μg | Compound | IM/IM |
| | | gC, | | 25 (IM) | |
| | | gD (2:1:1) | | | |

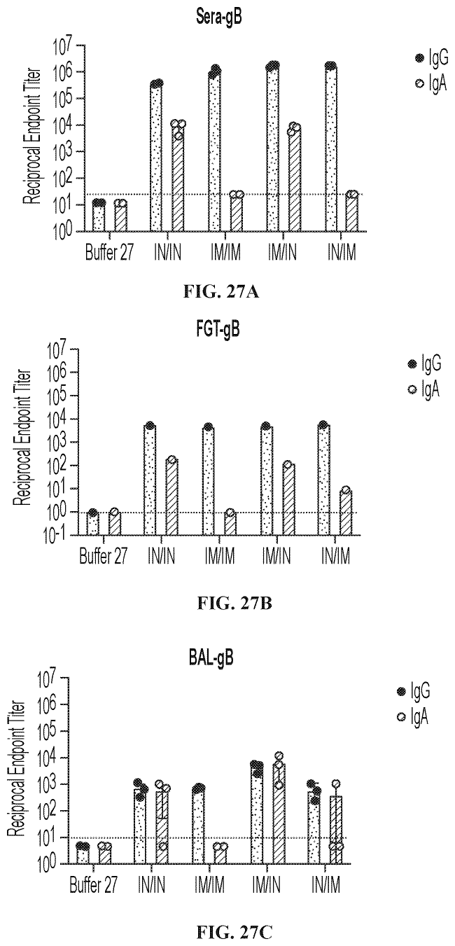

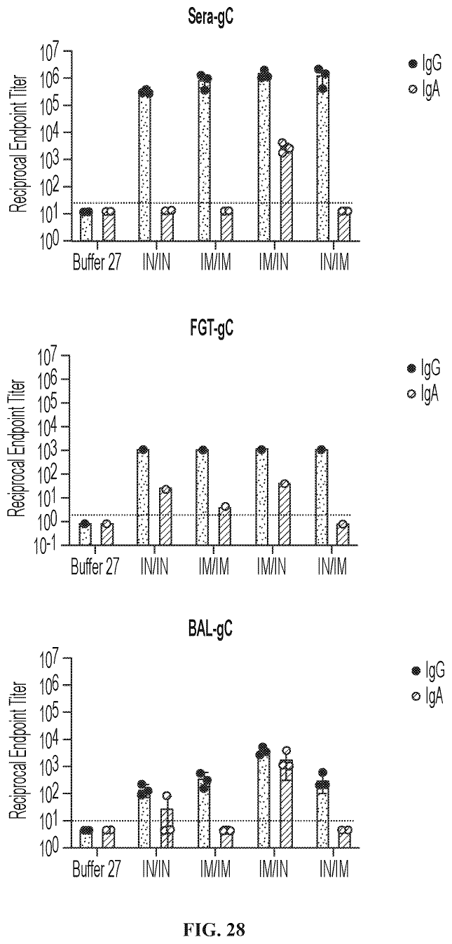

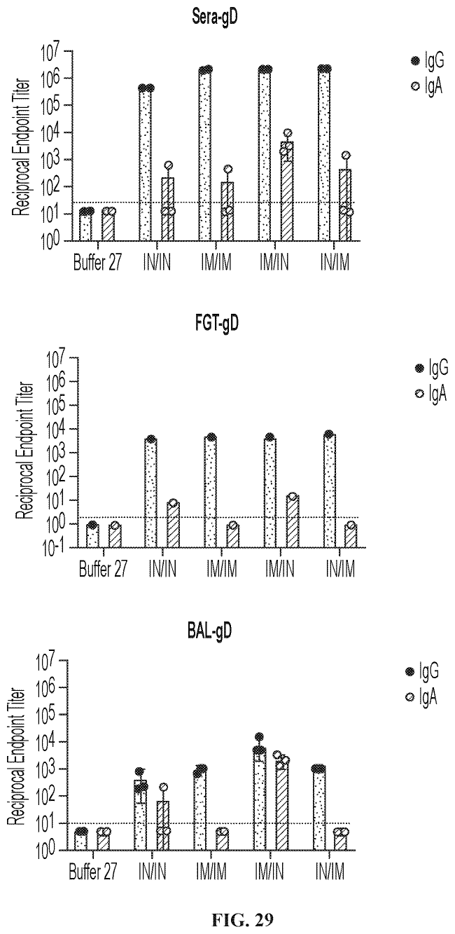

The results are shown in FIGS. 32-34. In particular, IN/IN and IM/IN vaccination protocols were found to induce IgA titers in mucosal and systemic compartments for gB (FIGS. 27A-7C), gC (FIG. 28), and gD (FIG. 29) at day 36.

### Example 23

### Immunogenicity and Efficacy of Intranasal Immunization in Guinea Pigs

The immunogenicity and expression of an mRNA vaccine against HSV-2 in guinea pigs is examined. Briefly, Guinea pigs are administered a prime dose of the vaccine on day 1 and a boost on day 35 (same dose as received on day 1) intranasally the experimental groups are shown in the following table:

TABLE 21

| Group | Treatment |
|---|---|
| 1 | PBS Control |
| 2 | Positive Control |
| 3 | Vaccine Composition IM |
| 4 | Vaccine Composition IN |

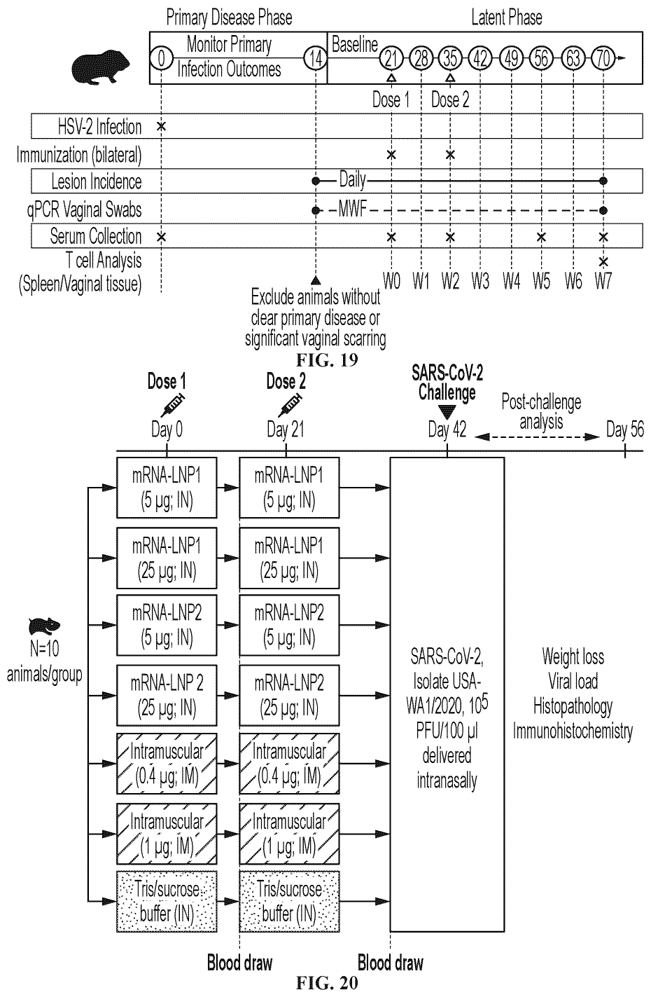

The study schedule is illustrated in FIG. 19. Guinea pigs are infected with HSV-2 on day 0 and monitored for 14 days. Guinea pigs without clear primary disease or significant vaginal scarring are excluded from the latent phase, which beings on day 14 and ends on day 70. During the latent phase, lesion incidence is evaluated daily. qPCR vaginal swabs are collected Mondays, Wednesdays, and Fridays during the latent phase to evaluate viral shedding frequency and load. Serum is collected on day 0 prior to virus exposure, day 21 prior to receiving the prime dose of the vaccine on day 21, day 35 prior to receiving the boost dose of the vaccine on day 35, and day 70 at the end of the study. At the end of the study, spleen and tissue samples are taken for T cell analysis.

### Example 24

### Production of Nanoparticle Compositions

Lipids are dissolved in ethanol at a concentration of 24 mg/mL and molar ratios of 49.0:11.2:39.3:0.5 (IL1:DSPC:cholesterol:PEG-DMG-2K) and mixed with the acidification buffer (45 mM acetate buffer at pH 4). The lipid solution and acidification buffer are mixed using a multi-inlet vortex mixer at a 3:7 volumetric ratio of lipid:buffer for mixer 1 and mixer 2 and a 1:3 volumetric ratio of lipid:buffer (25% ethanol) for mixer 3. After a 5 second residence time, the resulting nanoparticles are mixed with 55 mM sodium acetate at pH 5.6 at a volumetric ratio of 5:7 of nanoparticle:buffer. See Table 22 for mixing parameters. The resulting dilute nanoparticles are then buffer exchanged and concentrated using tangential flow filtration (TFF) into a final buffer containing 5 mM sodium acetate pH 5.0. See Table 23 for TFF parameters. Then a 70% sucrose solution in 5 mM acetate buffer at pH 5 is subsequently added.

TABLE 22

| | Mixer | | |
|---|---|---|---|
| Stream | 1 | 2 | 3 |
| 1 mL/min | 21 | 75 | 281 |
| 2 mL/min | 49 | 175 | 844 |
| 3 mL/min | 98 | 350 | 1575 |
| Nano-precipitation | 30% | 30% | 25% |
| (1 + 2) | | | |
| Mixed product | 12.5% | 12.5% | 10.4% |
| (1 + 2 + 3) | | | |

TABLE 23

| TFF Parameter | Mixer Value | |
|---|---|---|
| Feed flux | 240 | L/m²/hr |
| Loading | 100-300 | g/m³ |
| Initial concentration: Factor | 10x | |
| Diavolumes (DVs) | 8 | DVs |
| Final concentration: Factor | 4x | |
| TMP | 4-6 | psi |

The resulting nanoparticles at a lipid concentration of 7.33 mg/mL in 5 mM acetate (pH 5) and 75 g/L sucrose are mixed with mRNA at a concentration of 0.625 mg/mL in 42.5 mM sodium acetate pH 5.0, with N:P of 4.93. The mRNA solution and nanoparticles are mixed using a multi-inlet vortex mixer at a 3:2 volumetric ratio of nanoparticle:mRNA. Once the nanoparticles are loaded with mRNA, they undergo a 300 second residence time prior to addition of neutralization buffer containing 120 mM TRIS pH 8.12 at a ratio of 5:1 solution to buffer. Following this, PEG-DMG-2K dissolved in a 20 mM TRIS buffer (pH 7.5) is added to the neutralized nanoparticle solution at a ratio of 1:6, bringing the solution to the final molar ratios of IL1:DSPC:cholesterol:PEG-DMG-2K of 48.5:11.1:38.9:1.5%. This nanoparticle formulation is then modified with lipid amines. In a typical example, nanoparticle formulation at a concentration of 0.18 mg/mL mRNA and a 0.56 mL volume is modified with lipid amine SA3 (179.5 nmol) prepared in buffer containing 20 mM TRIS, 14.3 mM sodium acetate, 32 g/L sucrose and 140 mM NaCl—pH 7.5 at a volumetric ratio of 1:1 of nanoparticle:buffer. The resulting nanoparticle suspension is filtered through a 0.8/0.2 μm capsule filter and has an mRNA concentration of 0.09 mg/mL.

### Example 25

### Production of Nanoparticle Compositions

Lipid nanoparticles were prepared using ethanol drop nanoprecipitation followed by solvent exchange into suitable aqueous buffer using dialysis. An exemplary lipid nanoparticle composition can be prepared by a process where lipids are dissolved in ethanol at a concentration of 12.5 mM and molar ratios of 33:15:11:39.5:1.5 (e.g., ionizable lipid:SA46:phospholipid:cholesterol:PL1). Lipid to mRNA is maintained at a N/P ratio of 4.9. Then mRNA is diluted with 25 mM sodium acetate (pH 5.0) and combined with the lipid mixture at a volume ratio of 3:1 (aqueous: ethanol). Resulting formulations are dialyzed against 20 mM tris/8% sucrose/70 mM sodium chloride (pH 7.4) at a volume of 300 times that of the primary product using Slide-A-Lyzer dialysis cassettes (Thermo Scientific, Rockford, IL, USA) with a molecular cutoff of 10 KDa for at least 18 h. The first dialysis is carried out at room temperature in a digital orbital shaker (VWR, Radnor, PA, USA) at 85 rpm for 3 h and then dialyzed overnight at 4° C. Formulations are concentrated using Amicon ultra-centrifugal filters (EMD Millipore, Billerica, MA, USA), passed through a 0.22-μm filter and stored at 4° C. until use. Lipid nanoparticle solutions are typically adjusted to specific mRNA concentrations between 0.1 and 1 mg/mL.

### Example 26

### Production of Nanoparticle Compositions

Lipids are dissolved in ethanol at a concentration of 24 mg/mL and molar ratios of 49.0:11.2:39.3:0.5 (IL1:DSPC:cholesterol:PEG-DMG-2K) and mixed with the acidification buffer (45 mM acetate buffer at pH 4). The lipid solution and acidification buffer are mixed using a multi-inlet vortex mixer at a 3:7 volumetric ratio of lipid:buffer for mixer 1 and mixer 2 and a 1:3 volumetric ratio of lipid:buffer (25% ethanol) for mixer 3. After a 5 second residence time, the resulting nanoparticles are mixed with 55 mM sodium acetate at pH 5.6 at a volumetric ratio of 5:7 of nanoparticle:buffer. See Table 24 for mixing parameters. The resulting dilute nanoparticles are then buffer exchanged and concentrated using tangential flow filtration (TFF) into a final buffer containing 5 mM sodium acetate pH 5.0. See Table 25 for TFF parameters. Then a 70% sucrose solution in 5 mM acetate buffer at pH 5 is subsequently added.

TABLE 24
Mixing Parameters

| | Mixer | | |
|---|---|---|---|
| Stream | 1 | 2 | 3 |
| 1 mL/min | 21 | 75 | 281 |
| 2 mL/min | 49 | 175 | 844 |
| 3 mL/min | 98 | 350 | 1575 |
| Nano-precipitation (1 + 2) | 30% | 30% | 25% |
| Mixed product (1 + 2 + 3) | 12.5% | 12.5% | 10.4% |

TABLE 25
TFF Parameters

| TFF Parameter | Mixer Value | |
|---|---|---|
| Feed flux | 240 | L/m²/hr |
| Loading | 100-300 | g/m³ |
| Initial concentration: Factor | 10x | |
| Diavolumes (DVs) | 8 | DVs |
| Final concentration: Factor | 4x | |
| TMP | 4-6 | psi |

The resulting nanoparticles at a lipid concentration of 7.33 mg/mL in 5 mM acetate (pH 5) and 75 g/L sucrose are mixed with mRNA (luciferease) at a concentration of 0.625 mg/mL in 42.5 mM sodium acetate pH 5.0, with N:P of 4.93. The nanoparticle solution and nanoparticles are mixed using a multi-inlet vortex mixer at a 3:2 volumetric ratio of nanoparticle:mRNA. Once loaded with mRNA, these intermediate nanoparticles undergo a 300 second residence time prior addition of neutralization buffer containing 120 mM TRIS pH 8.12 at a volumetric ratio of 5:1 of nanoparticle:buffer.

For HeLa studies evaluating luciferase protein expression, PEG-DMG-2K dissolved in a 20 mM TRIS buffer (pH 7.5) is added to the neutralized intermediate nanoparticle solution at a ratio of 1:6, bringing the solution to the final molar ratios of IL1:DSPC:cholesterol:PEG-DMG-2K of 48.5:11.1:38.9:1.5%. This nanoparticle formulation is then modified with lipid amines. In a typical example, nanoparticle formulation at a concentration of 0.18 mg/mL mRNA and a 0.56 mL volume is modified with lipid amine SA50 (467.2 nmol) prepared in buffer containing 20 mM IRIS, 14.3 mM sodium acetate, 32 g/L sucrose and 140 mM NaCl—pH 7.5 at a volumetric ratio of 1:1 of nanoparticle: buffer.

Resulting nanoparticle suspensions are filtered through a 0.8/0.2 μm capsule filter and filled into glass vials at an mRNA strength of about 0.1-1 mg/mL Biophysical data (Diameter and PDI from DLS measurements and % Encapsulation using Ribogreen assay) for luciferase mRNA nanoparticles with lipid amines is shown in Table 26.

TABLE 26
Luciferase mRNA nanoparticle biophysical data

| SA | Diameter | | % Encapsulation |
|---|---|---|---|
| # | (nm) | PDI | (Ribogreen) |
| SA59 | 74 | 0.15 | 99.7 |
| SA60 | 74 | 0.13 | 98.8 |
| SA61 | 77 | 0.16 | 99.1 |
| SA62 | 78 | 0.20 | 99.0 |
| SA66 | 72 | 0.18 | 88.2 |
| SA69 | 71 | 0.16 | 98.9 |
| SA70 | 76 | 0.21 | 97.9 |
| SA71 | 73 | 0.15 | 99.1 |
| SA72 | 72 | 0.11 | 99.1 |
| SA74 | 75 | 0.19 | 99.1 |
| SA75 | 74 | 0.16 | 98.8 |
| SA76 | 75 | 0.20 | 98.1 |
| SA77 | 74 | 0.20 | 98.2 |
| SA78 | 76 | 0.20 | 99.8 |
| SA79 | 74 | 0.18 | 97.0 |
| SA81 | 72 | 0.12 | 99.8 |
| SA82 | 73 | 0.11 | 99.1 |
| SA83 | 75 | 0.14 | 99.3 |
| SA84 | 74 | 0.16 | 99.9 |
| SA87 | 75 | 0.18 | 98.2 |
| SA88 | 75 | 0.13 | 99.8 |
| SA89 | 77 | 0.16 | 99.7 |
| SA90 | 76 | 0.19 | 99.6 |
| SA95 | 75 | 0.20 | 99.7 |
| SA96 | 71 | 0.15 | 99.6 |
| SA97 | 72 | 0.16 | 98.8 |
| SA98 | 70 | 0.15 | 97.0 |
| SA110 | 70 | 0.14 | 98.8 |
| SA111 | 76 | 0.18 | 99.6 |
| SA113 | 77 | 0.23 | 99.7 |
| SA114 | 75 | 0.18 | 99.7 |
| SA116 | 73 | 0.20 | 97.6 |
| SA117 | 77 | 0.28 | 99.7 |
| SA118 | 74 | 0.19 | 99.7 |
| SA119 | 78 | 0.18 | 99.4 |
| SA120 | 76 | 0.28 | 99.1 |
| SA122 | 75 | 0.15 | 99.4 |
| SA123 | 73 | 0.14 | 99.5 |
| SA124 | 74 | 0.14 | 99.8 |
| SA125 | 77 | 0.19 | 99.7 |
| SA126 | 75 | 0.18 | 99.7 |
| SA127 | 74 | 0.17 | 99.7 |
| SA128 | 75 | 0.16 | 99.4 |
| SA129 | 70 | 0.16 | 99.5 |
| SA131 | 72 | 0.12 | 99.6 |
| SA132 | 81 | 0.41 | 99.3 |
| SA133 | 73 | 0.17 | 99.0 |
| SA134 | 72 | 0.13 | 99.5 |
| SA135 | 71 | 0.12 | 99.3 |
| SA136 | 75 | 0.16 | 99.7 |
| SA137 | 79 | 0.20 | 99.6 |
| SA138 | 72 | 0.19 | 99.2 |
| SA139 | 72 | 0.12 | 99.4 |
| SA141 | 73 | 0.15 | 98.9 |
| SA142 | 72 | 0.19 | 99.5 |
| SA144 | 71 | 0.15 | 99.2 |
| SA145 | 77 | 0.17 | 99.7 |
| SA149 | 75 | 0.15 | 99.5 |
| SA151 | 72 | 0.12 | 95.8 |
| SA152 | 78 | 0.15 | 98.2 |
| SA153 | 72 | 0.15 | 95.8 |
| SA154 | 73 | 0.18 | 99.6 |
| SA155 | 73 | 0.11 | 98.9 |
| SA156 | 74 | 0.15 | 99.5 |
| SA157 | 75 | 0.13 | 99.6 |
| SA158 | 79 | 0.28 | 99.7 |
| SA160 | 75 | 0.11 | 99.7 |
| SA161 | 77 | 0.16 | 99.6 |
| SA162 | 75 | 0.15 | 99.4 |
| SA163 | 71 | 0.17 | 99.1 |
| SA164 | 73 | 0.14 | 98.0 |
| SA165 | 72 | 0.13 | 98.8 |
| SA166 | 75 | 0.14 | 99.2 |
| SA167 | 75 | 0.15 | 98.7 |
| SA168 | 79 | 0.16 | 99.3 |
| SA169 | 77 | 0.20 | 99.4 |
| SA170 | 75 | 0.21 | 99.6 |
| SA171 | 73 | 0.23 | 99.2 |
| SA172 | 74 | 0.10 | 99.4 |
| SA173 | 73 | 0.18 | 99.5 |
| SA174 | 73 | 0.17 | 99.5 |
| SA178 | 75 | 0.20 | 98.8 |
| SA179 | 73 | 0.17 | 99.0 |
| SA180 | 76 | 0.17 | 98.7 |
| SA181 | 74 | 0.16 | 99.5 |
| SA182 | 73 | 0.14 | 99.0 |
| SA183 | 76 | 0.21 | 99.6 |
| SA184 | 74 | 0.12 | 98.8 |
| SA185 | 75 | 0.16 | 99.4 |
| SA186 | 73 | 0.14 | 99.3 |
| SA187 | 73 | 0.17 | 99.0 |
| SA188 | 80 | 0.13 | 99.6 |
| SA189 | 78 | 0.33 | 98.1 |

### Example 27

### Protein Expression in Human Cervical Cancer Epithelial Cell (HeLa)

Lipid nanoparticle compositions were prepared in a manner analogous to that in Example 27. To evaluate LNP cellular uptake and protein expression in vitro, HeLa cells from ATCC.org (ATCC CCL-2) are used. The cells are cultured in complete Minimum Essential Medium (MEM) and are plated in 96 well Cell Carrier Ultra plate with PDL coated surface (PerkinElmer) prior to running an experiment.

### Example 28

### Luciferase Protein Expression Assay in HeLa Cells

Cells were transfected with buffer control (PBS) or LNPs encapsulating Luciferase mRNA (25 ng per well; N=4 replicate wells) in serum-free MEM media. LNP transfected cells were incubated for 5 h, followed by media removal and supplementation with complete MEM media. Cells were further incubated in complete MEM media overnight (24 h). Following the 24 hr incubation, luciferase protein expression was measured using the ONE-Glo™ Luciferase Assay (Promega). Cells were lysed using 1× Passive Lysis Buffer (Cat. #E194A) for 10 min in a microplate mixer at room temperature. Luciferase in the supernatant was measured by adding Luciferase Assay Reagent (Cat. #E151A) containing luciferin. Bioluminescence was then immediately measured on a Synergy H1 plate reader (BioTek). The results shown in Table 27 show the Average Relative Light Units (RLU) of each sample tested.

TABLE 27
Luciferase Expression Results

| SA | Average Relative Light Units ± |
|---|---|
| # | Standard Deviation |
| SA59 | 17590.1 ± 6267.4 |
| SA60 | 30508.5 ± 6869.7 |
| SA61 | 14343.1 ± 5298.1 |
| SA62 | 18807.1 ± 6618.4 |
| SA66 | 13951.3 ± 6740.3 |
| SA69 | 35256.9 ± 8309.9 |
| SA70 | 11822.1 ± 4128.6 |
| SA71 | 32758.9 ± 5897.1 |
| SA72 | 30826.5 ± 5913.2 |
| SA74 | 23899.2 ± 6943.7 |
| SA75 | 37732.4 ± 6382.7 |
| SA76 | 38232.8 ± 4603.5 |
| SA77 | 39191.9 ± 4319 |
| SA78 | 14341.8 ± 3497.6 |
| SA79 | 105.1 ± 20 |
| SA81 | 19619.8 ± 4362.6 |
| SA82 | 33396.2 ± 4902.7 |
| SA83 | 35394.8 ± 5033.2 |
| SA84 | 29639.9 ± 7554 |
| SA87 | 13346.1 ± 2590.8 |
| SA88 | 17105.3 ± 7097.3 |
| SA89 | 8209.2 ± 4138.5 |
| SA90 | 9337.4 ± 3055.2 |
| SA95 | 22952.2 ± 5937.7 |
| SA96 | 17172.8 ± 2176 |
| SA97 | 13144.4 ± 4350.5 |
| SA98 | 80.7 ± 36.7 |
| SA110 | 14227 ± 2513.9 |
| SA111 | 26647.6 ± 9522.1 |
| SA113 | 20665.4 ± 7510.5 |
| SA114 | 22594.3 ± 9128.4 |
| SA116 | 110.9 ± 26.9 |
| SA117 | 10134.1 ± 3860.2 |
| SA118 | 12261.7 ± 4328.5 |
| SA119 | 18979.6 ± 7864.8 |
| SA120 | 34695.5 ± 9492.2 |
| SA122 | 29605.6 ± 6326.9 |
| SA123 | 28520.5 ± 6429.4 |
| SA124 | 45805.9 ± 5630.6 |
| SA125 | 15625.8 ± 3399.4 |
| SA126 | 31706.7 ± 7386.3 |
| SA127 | 41246.9 ± 5672.4 |
| SA128 | 39386.4 ± 5261.4 |
| SA129 | 144.8 ± 21.6 |
| SA131 | 40915.5 ± 4983.3 |
| SA132 | 131.6 ± 15.5 |
| SA133 | 26696.2 ± 5831.4 |
| SA134 | 29194.3 ± 6336.9 |
| SA135 | 27499.8 ± 7269.5 |
| SA136 | 16245.8 ± 6554.6 |
| SA137 | 15239.8 ± 6248.1 |
| SA138 | 22039.8 ± 8550.1 |
| SA139 | 24497.9 ± 3998.9 |
| SA141 | 35801.9 ± 8884.3 |
| SA142 | 44925.4 ± 6006.5 |
| SA144 | 33357.5 ± 5288.8 |
| SA145 | 36033.7 ± 4254.5 |
| SA149 | 31035.2 ± 6333.7 |
| SA151 | 36345.5 ± 6730.7 |
| SA152 | 41692.8 ± 9336.8 |
| SA153 | 35859.3 ± 5894.9 |
| SA154 | 28128.6 ± 4655.3 |
| SA155 | 31922.9 ± 8053.8 |
| SA156 | 37662.9 ± 8258.6 |
| SA157 | 24130.4 ± 4741.7 |
| SA158 | 33097.8 ± 7405.1 |
| SA160 | 19694.4 ± 6954.5 |
| SA161 | 17839.3 ± 7760 |
| SA162 | 34454.6 ± 3101.8 |
| SA163 | 30168 ± 7878.9 |
| SA164 | 17129.2 ± 2374.1 |
| SA165 | 33585.6 ± 7766.5 |
| SA166 | 33377.3 ± 8879.8 |
| SA167 | 38742.4 ± 6037.5 |
| SA168 | 35639.5 ± 6688.9 |
| SA169 | 28034.9 ± 4050.3 |
| SA170 | 23687.3 ± 3865 |
| SA171 | 22232.4 ± 9084.1 |
| SA172 | 27528.4 ± 6654.4 |
| SA173 | 33596.1 ± 6033 |
| SA174 | 31280.7 ± 5830.7 |
| SA178 | 10196.5 ± 602.4 |
| SA179 | 12298.9 ± 928.6 |
| SA180 | 35476 ± 6770.8 |
| SA181 | 12107.3 ± 1481.4 |
| SA182 | 38564.9 ± 12453 |
| SA183 | 33915.2 ± 6639.9 |
| SA184 | 26222.4 ± 4612.1 |
| SA185 | 36168.3 ± 7638.9 |
| SA186 | 39332.2 ± 8317.2 |
| SA187 | 23953.8 ± 3430 |
| SA188 | 19740.5 ± 4837 |
| SA189 | 386 ± 44.3 |

### Example 29

### Intranasal Messenger RNA (mRNA)-Lipid Nanoparticle (LNP) Vaccination Protects Hamsters from SARS-CoV-2 Infection

Intranasal vaccination represents a promising approach for preventing disease caused by respiratory pathogens by eliciting a mucosal immune response in the respiratory tract that may act as an early barrier to infection and transmission. This Example investigated immunogenicity and protective efficacy of intranasally administered messenger RNA (mRNA)-lipid nanoparticle (LNP) encapsulated vaccines against severe acute respiratory syndrome coronavirus 2 (SARS-CoV-2) in Syrian golden hamsters. Intranasal mRNA-LNP vaccination systemically induced spike-specific binding (IgG and IgA) and neutralizing antibodies with similar robustness as to intramuscular controls. Additionally, intranasal vaccination also decreased viral loads in the respiratory tract, reduced lung pathology, and prevented weight loss after SARS-CoV-2 challenge.

### INTRODUCTION

Disease caused by respiratory pathogens remains a pre-eminent threat to global public health.¹ With over 600 million cases and 6.5 million deaths reported worldwide as of November 2022, the ongoing coronavirus disease 2019 (COVID-19) pandemic caused by severe acute respiratory syndrome coronavirus 2 (SARS-CoV-2) is the most current and vivid example of the impact of respiratory diseases on global populations.² Prior to the COVID-19 pandemic, upper and lower respiratory tract infections were responsible for over 17.7 billion cases and 2.5 million deaths globally, and primarily caused by viruses and bacteria such as Streptococcus pneumoniae, respiratory syncytial virus, and influenza virus.³ There remains a continual risk of emerging respiratory infectious diseases,⁴ as evidenced by evolving SARS-CoV-2 variants in the current COVID-19 pandemic as well as by notable prior pandemics caused by pathogens such as influenza virus.² Vaccination remains a pivotal strategy to address infectious disease-related morbidity and mortality,⁵ with a need for innovative vaccination strategies and technologies that can be deployed quickly and establish robust local mucosal immune responses in the upper respiratory tract to impede infection and transmission.^{6,7}

Currently, most licensed vaccines against respiratory diseases are administered intramuscularly, which robust systemic immunity but elicit suboptimal immunity at the mucosal sites targeted by respiratory pathogens.⁶⁻⁹ Intranasal vaccination can induce both systemic and local mucosal immune responses, and is a promising approach to combat respiratory pathogens as it has the potential to limit infection and minimize transmission.^{7,9-14} Further, this approach could increase vaccination rates and compliance with recommended schedules, as its minimally invasive delivery may facilitate administration without the need for trained healthcare personnel.^{7,15,16} Additionally, intranasal vaccination by an aerosolizing device could potentially bypass injection-associated phobias that are a known factors for vaccine hesitancy.¹⁷

Messenger RNA (mRNA)-lipid nanoparticle (LNP) encapsulated vaccines have already demonstrated the ability to protect against infectious respiratory pathogens, as shown by currently available COVID-19 vaccines: mRNA-1273 (Spikevax; Moderna, Inc., Cambridge, MA, USA²⁰) and BNT162b2 (Comirnaty; Pfizer Inc, New York, NY, USA; BioNTech Manufacturing GmbH, Mainz, Germany).²¹⁻²⁶ Moreover, mRNA-LNP vaccines encode only targeted proteins and therefore do not induce a vector-specific immune response and thus have the potential for repeat administration without diminishment of effect caused by anti-vector immunity,^{27,28} mRNA is also non-infectious and non-integrative.²⁹ Further, LNPs have potential for delivery of mRNA to specific cells, tissues, and organs.^{30,31}

In this Example, a 2-dose regimen of intranasally administered mRNA-based SARS-CoV-2 vaccines is demonstrated to be immunogenic and to protect against viral infection in a Syrian golden hamster model.

### Results

Intranasal mRNA-LNP Vaccination Induces Binding and Neutralizing Antibody Responses in Sera

To assess the immunogenic potential of intranasally administered N₁-methyl-pseudourine-modified mRNA-LNPs, SARS-CoV-2 vaccines formulated with two different LNP compositions: mRNA-LNP1 and mRNA-LNP2 were developed. mRNA-LNP1 is similar in composition to the LNP used in mRNA-1273, with analogous but chemically distinct ionizable lipids, and mRNA-LNP2 is a composition further developed for improved respiratory tract delivery. All vaccines encoded a prefusion-stabilized SARS-CoV-2 spike (S) protein, stabilized with six proline mutations (protein, SEQ ID NO: 5; ORF, SEQ ID NO: 3; Table 36).³² Syrian golden hamsters (n=10 per group) were vaccinated three weeks apart with 2 doses of either mRNA-LNP vaccines at 5 μg or 25 μg or with tris/sucrose buffer (mock-vaccinated) via the intranasal rout (Days 0 and 21; FIG. 20). For comparison purposes, two groups of hamsters were intramuscularly vaccinated with 0.4 μg or 1 μg of vaccine with the same mRNA included in the intranasal compositions but formulated with the preclinical version of the same LNP utilized in injectable mRNA-1273. Immunogenicity was assessed at 3 weeks after dose 1 (Day 21) and 3 weeks after dose 2 (Day 41); S-specific serum immunoglobulin (Ig) G or A binding antibody responses were measured by enzyme-linked immunosorbent assay (ELISA) and serum neutralizing antibody titers were measured by a plaque reduction neutralization test (PRNT).

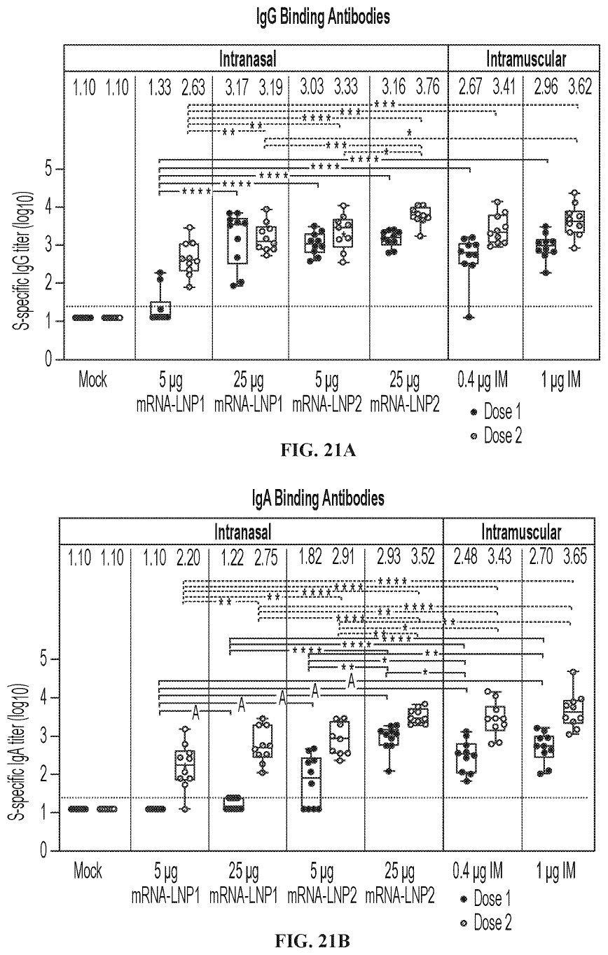

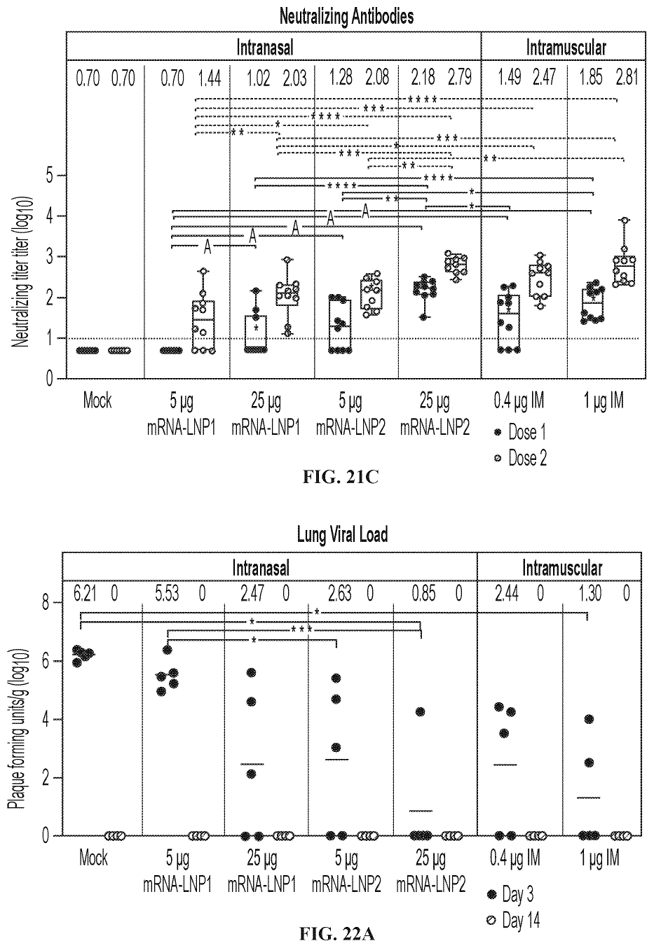

Three weeks after the first dose, both intranasal vaccines (25 μg dose level) elicited S-specific serum IgG binding titers comparable to those induced by intramuscular administration (0.4 μg and 1 μg). At the 5-μg dose level, mRNA-LNP2 induced similar titers to the 25-μg dose level and to intramuscular controls; (0.4 μg and 1 μg); mRNA-LNP2 titers at this lower dose level were significantly higher than mRNA-LNP1 titers (adjusted P<0.0001; FIG. 21A; Table 28). After the second dose, S-specific IgG titers generally increased across all vaccine groups and dose levels, with mRNA-LNP2 eliciting significantly higher titers than mRNA-LNP1 at the corresponding dose levels (5 μg, adjusted P<0.01; 25 μg, adjusted P<0.001).

TABLE 28
Statistical comparisons of S-specific serum binding
IgG antibody titers after vaccination

| | | |
|---|---|---|
| | Estimated | Adjusted |
| | Fold-change | P- |
| Comparison | (95% CI) | value |

Dose 1 (Day 21)

| | | |
|---|---|---|
| 25 μg mRNA-LNP1 over 5 μg mRNA-LNP1 | 68.53 | <1e−6 |
| | (25.63-185.69) | |
| 25 μg mRNA-LNP2 over 5 μg mRNA-LNP1 | 67.68 | <1e−6 |
| | (25.04-182.43) | |
| 5 μg mRNA-LNP2 over 5 μg mRNA-LNP1 | 50.61 | <1e−6 |
| | (22.1-117.24) | |
| 25 μg mRNA-LNP1 over 0.4 μg IM | 3.11 | 0.9619 |
| | (0.86-11.06) | |
| 25 μg mRNA-LNP1 over 1 μg IM | 1.63 | 0.8426 |
| | (0.61-4.38) | |
| 5 μg mRNA-LNP1 over 0.4 μg IM | 0.05 | <1e−6 |
| | (0.02-0.14) | |
| 5 μg mRNA-LNP1 over 1 μg IM | 0.02 | <1e−6 |
| | (0.01-0.05) | |
| 25 μg mRNA-LNP2 over 25 μg mRNA-LNP1 | 0.99 | 0.4892 |
| | (0.31-3.14) | |
| 25 μg mRNA-LNP2 over 5 μg mRNA-LNP2 | 1.34 | 0.7183 |
| | (0.5-3.71) | |
| 25 μg mRNA-LNP2 over 0.4 μg IM | 3.07 | 0.9635 |
| | (0.9-11.02) | |
| 25 μg mRNA-LNP2 over 1 μg IM | 1.61 | 0.8312 |
| | (0.6-4.4) | |
| 5 μg mRNA-LNP2 over 25 μg mRNA-LNP1 | 0.74 | 0.2668 |
| | (0.27-2.03) | |
| 5 μg mRNA-LNP2 over 0.4 μg IM | 2.3 | 0.9359 |
| | (0.75-6.89) | |
| 5 μg mRNA-LNP2 over 1 μg IM | 1.2 | 0.6883 |
| | (0.54-2.69) | |
| 1 μg IM over 0.4 μg IM | 1.91 | 0.8853 |
| | (0.63-5.83) | |

Dose 2 (Day 42)

| | | |
|---|---|---|
| 25 μg mRNA-LNP1 over 5 μg mRNA-LNP1 | 3.64 | 0.0024 |
| | (1.54-8.55) | |
| 25 μg mRNA-LNP2 over 25 μg mRNA-LNP1 | 3.68 | 6e−04 |
| | (1.78-7.55) | |
| 25 μg mRNA-LNP2 over 5 μg mRNA-LNP1 | 13.38 | <1e−6 |
| | (5.58-31.97) | |
| 25 μg mRNA-LNP2 over 5 μg mRNA-LNP2 | 2.7 | 0.0156 |
| | (1.1-6.61) | |
| 5 μg mRNA-LNP2 over 5 μg mRNA-LNP1 | 4.95 | 0.0023 |
| | (1.8-13.8) | |
| 25 μg mRNA-LNP1 over 0.4 μg IM | 0.61 | 0.1004 |
| | (0.27-1.34) | |
| 25 μg mRNA-LNP1 over 1 μg IM | 0.37 | 0.0138 |
| | (0.17-0.86) | |
| 5 μg mRNA-LNP1 over 0.4 μg IM | 0.17 | 2e−04 |
| | (0.06-0.43) | |
| 5 μg mRNA-LNP1 over 1 μg IM | 0.1 | 2e−04 |
| | (0.04-0.27) | |
| 25 μg mRNA-LNP2 over 0.4 μg IM | 2.24 | 0.9742 |
| | (0.99-4.93) | |
| 25 μg mRNA-LNP2 over 1 μg IM | 1.38 | 0.7796 |
| | (0.61-3.2) | |
| 5 μg mRNA-LNP2 over 25 μg mRNA-LNP1 | 1.36 | 0.7592 |
| | (0.56-3.32) | |
| 5 μg mRNA-LNP2 over 0.4 μg IM | 0.83 | 0.3406 |
| | (0.32-2.27) | |
| 5 μg mRNA-LNP2 over 1 μg IM | 0.51 | 0.0894 |
| | (0.19-1.38) | |
| 1 μg IM over 0.4 μg IM | 1.63 | 0.8621 |
| | (0.63-3.98) | |

CI, confidence interval;
IgG, immunoglobulin G;
IM, intramuscular;
LNP, lipid nanoparticle;
mRNA, messenger RNA.

A single intranasal administration of mRNA-LNP2 (5 μg and 25 μg) elicited S-specific serum IgA binding antibody titers in sera (FIG. 21B), with the 25-μg dose level eliciting higher (adjusted P<0.05) or similar titers as intramuscular administration (0.4 μg and 1 μg, respectively; Table 29). mRNA-LNP1 at the 5 μg and 25 μg dose levels elicited lower titers than intramuscular controls and respective mRNA-LNP2 doses. A second dose of either intranasal vaccine composition increased IgA binding titers, with the 25 μg dose levels eliciting significantly higher titers than the respective 5-μg dose level (adjusted P<0.01). Additionally, mRNA-LNP2 at the 25-μg dose level elicited comparable IgA titers to intramuscular administration (0.4 μg and 1 μg) after either dose.

TABLE 29
Statistical comparisons of S-specific serum binding
IgA antibody titers after vaccination

| | | |
|---|---|---|
| | Estimated Fold- | Adjusted |
| Comparison | change (95% CI) | P-value |

Dose 1 (Day 21)

| | | |
|---|---|---|
| 25 μg mRNA-LNP2 over 25 μg | 83.81 | <1e−6 |
| mRNA-LNP1 | (38.04-224.25) | |
| 25 μg mRNA-LNP2 over 5 μg | 32.7 | 0.0035 |
| mRNA-LNP2 | (3.92-716.22) | |
| 25 μg mRNA-LNP2 over 0.4 μg IM | 2.84 | 0.0104 |
| | (1.18-6.73) | |
| 25 μg mRNA-LNP1 over 0.4 μg IM | 0.03 | <1e−6 |
| | (0.01-0.09) | |
| 25 μg mRNA-LNP1 over 1 μg IM | 0.02 | <1e−6 |
| | (0.01-0.05) | |
| 25 μg mRNA-LNP2 over 1 μg IM | 1.7 | 0.8993 |
| | (0.72-3.93) | |
| 5 μg mRNA-LNP2 over 25 μg | 2.56 | 0.8043 |
| mRNA-LNP1 | (0.12-24.18) | |
| 5 μg mRNA-LNP2 over 0.4 μg IM | 0.09 | 0.0171 |
| | (<1e−6-0.8) | |
| 5 μg mRNA-LNP2 over 1 μg IM | 0.05 | 0.0066 |
| | (<1e−6-0.44) | |
| 1 μg IM over 0.4 μg IM | 1.67 | 0.8746 |
| | (0.67-4.14) | |

Dose 2 (Day 42)

| | | |
|---|---|---|
| 25 μg mRNA-LNP1 over 5 μg | 3.74 | 0.0051 |
| mRNA-LNP1 | (1.37-10.19) | |
| 25 μg mRNA-LNP2 over 25 μg | 5.77 | <1e−6 |
| mRNA-LNP1 | (2.57-12.95) | |
| 25 μg mRNA-LNP2 over 5 μg | 21.57 | <1e−6 |
| mRNA-LNP1 | (7.93-60.5) | |
| 25 μg mRNA-LNP2 over 5 μg | 4.05 | 0.0066 |
| mRNA-LNP2 | (1.37-11.73) | |
| 5 μg mRNA-LNP2 over 5 μg | 5.33 | 0.006 |
| mRNA-LNP1 | (1.55-18.18) | |
| 25 μg mRNA-LNP1 over 0.4 μg IM | 0.21 | 0.0025 |
| | (0.08-0.52) | |
| 25 μg mRNA-LNP1 over 1 μg IM | 0.13 | <1e−6 |
| | (0.05-0.33) | |
| 5 μg mRNA-LNP1 over 0.4 μg IM | 0.06 | <1e−6 |
| | (0.02-0.18) | |
| 5 μg mRNA-LNP1 over 1 μg IM | 0.03 | <1e−6 |
| | (0.01-0.1) | |
| 25 μg mRNA-LNP2 over 0.4 μg IM | 1.21 | 0.6658 |
| | (0.47-3.09) | |
| 25 μg mRNA-LNP2 over 1 μg IM | 0.73 | 0.2411 |
| | (0.28-1.88) | |
| 5 μg mRNA-LNP2 over 25 μg | 1.43 | 0.7531 |
| mRNA-LNP1 | (0.51-4.12) | |
| 5 μg mRNA-LNP2 over 0.4 μg IM | 0.3 | 0.0219 |
| | (0.09-0.95) | |
| 5 μg mRNA-LNP2 over 1 μg IM | 0.18 | 0.0044 |
| | (0.06-0.57) | |
| 1 μg IM over 0.4 μg IM | 1.67 | 0.8428 |
| | (0.58-4.79) | |

CI, confidence interval;
IgA, immunoglobulin A;
IM, intramuscular;
LNP, lipid nanoparticle;
mRNA, messenger RNA.

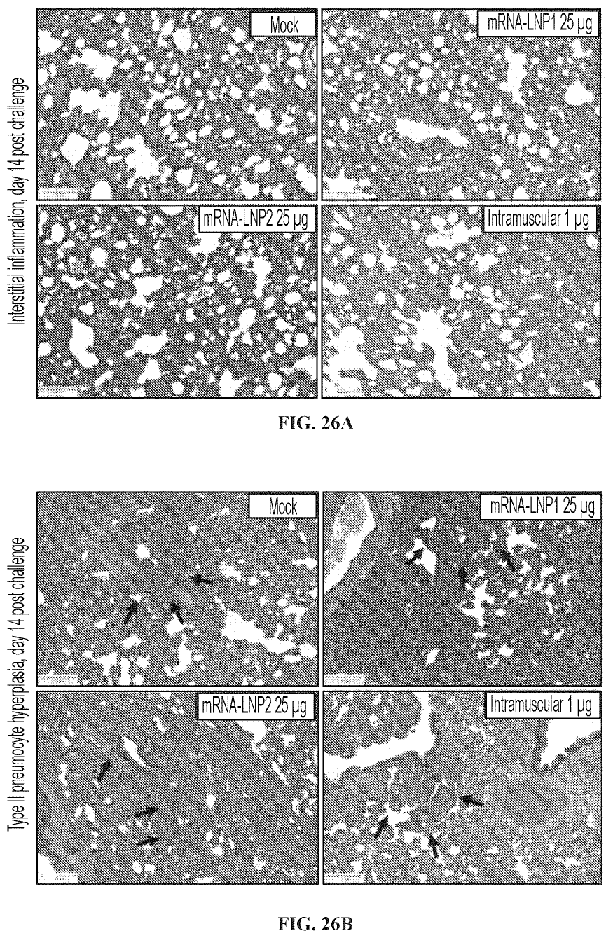

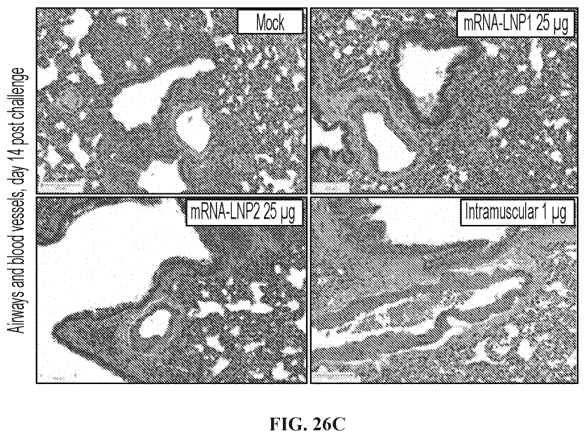

In addition to S-specific binding titers, neutralizing antibody responses in sera were evaluated (FIG. 26C). Three weeks after the first dose, titers were variable after mRNA-LNP1 (25-μg dose), mRNA-LNP2 (5-μg dose), or intramuscular administration (0.4-μg dose), with titers not detected in all hamsters. No hamsters administered mRNA-LNP1 5 μg had detectable titers after the first dose. However, mRNA-LNP2 (25 μg dose) elicited neutralizing antibody titers in all vaccinated hamsters and titers were significantly higher (P<0.05) or comparable to intramuscular controls (0.5 1 μg, respectively; Table 30). Neutralizing antibody titers increased for all vaccine groups after the second dose. mRNA-LNP2 (5 μg and 25 μg) induced significantly higher titers than mRNA-LNP1 at the respective dose level (5 μg, adjusted P<0.05; 25 g, adjusted P<0.001). Two doses of mRNA-LNP2 at either dose level induced similar neutralizing titers to intramuscular vaccination (0.4 μg and 1 μg).

TABLE 30
Statistical comparisons of serum neutralizing
antibody titers after vaccination

| | | |
|---|---|---|
| | Estimated Fold- | Adjusted |
| Comparison | change (95% CI) | P-value |

Dose 1 (Day 21)

| | | |
|---|---|---|
| 25 μg mRNA-LNP2 over 25 μg | 38.84 | <1e−6 |
| mRNA-LNP1 | (8.7-287.37) | |
| 25 μg mRNA-LNP2 over 5 μg | 17.46 | 0.0044 |
| mRNA-LNP2 | (2.21-275.24) | |
| 25 μg mRNA-LNP2 over 0.4 μg IM | 8.09 | 0.0205 |
| | (1.09-96.32) | |
| 25 μg mRNA-LNP1 over 0.4 μg IM | 0.21 | 0.0801 |
| | (0.02-2.64) | |
| 25 μg mRNA-LNP1 over 1 μg IM | 0.06 | <1e−6 |
| | (0.01-0.21) | |
| 25 μg mRNA-LNP2 over 1 μg IM | 2.16 | 0.8996 |
| | (0.63-7.5) | |
| 5 μg mRNA-LNP2 over 25 μg | 2.22 | 0.7781 |
| mRNA-LNP1 | (0.12-24.4) | |
| 5 μg mRNA-LNP2 over 0.4 μg IM | 0.46 | 0.275 |
| | (0.02-7.72) | |
| 5 μg mRNA-LNP2 over 1 μg IM | 0.12 | 0.0149 |
| | (0.01-0.77) | |
| 1 μg IM over 0.4 μg IM | 3.74 | 0.9309 |
| | (0.6-39.29) | |

Dose 2 (Day 42)

| | | |
|---|---|---|
| 25 μg mRNA-LNP1 over 5 μg | 5.07 | 0.0054 |
| mRNA-LNP1 | (1.53-18.22) | |
| 25 μg mRNA-LNP2 over 25 μg | 5.7 | 0.0008 |
| mRNA-LNP1 | (2.28-14.07) | |
| 25 μg mRNA-LNP2 over 5 μg | 28.89 | <1e−6 |
| mRNA-LNP1 | (8.5-111.28) | |
| 25 μg mRNA-LNP2 over 5 μg | 5.08 | 0.0059 |
| mRNA-LNP2 | (1.45-18.06) | |
| 5 μg mRNA-LNP2 over 5 μg | 5.69 | 0.0119 |
| mRNA-LNP1 | (1.32-27.13) | |
| 25 μg mRNA-LNP1 over 0.4 μg IM | 0.36 | 0.0173 |
| | (0.14-0.91) | |
| 25 μg mRNA-LNP1 over 1 μg IM | 0.17 | 0.0004 |
| | (0.06-0.45) | |
| 5 μg mRNA-LNP1 over 0.4 μg IM | 0.07 | 0.0002 |
| | (0.02-.024) | |
| 5 μg mRNA-LNP1 over 1 μg IM | 0.03 | <1e−6 |
| | (0.01-0.12) | |
| 25 μg mRNA-LNP2 over 0.4 μg IM | 2.06 | 0.94 |
| | (0.83-5.26) | |
| 25 μg mRNA-LNP2 over 1 μg IM | 0.96 | 0.4737 |
| | (0.33-2.65) | |
| 5 μg mRNA-LNP2 over 25 μg | 1.12 | 0.5776 |
| mRNA-LNP1 | (0.32-3.86) | |
| 5 μg mRNA-LNP2 over 0.4 μg IM | 0.41 | 0.0758 |
| | (0.12-1.43) | |
| 5 μg mRNA-LNP2 over 1 μg IM | 0.19 | 0.0065 |
| | (0.05-0.69) | |
| 1 μg IM over 0.4 μg IM | 2.15 | 0.9333 |
| | (0.77-6.24) | |

CI, confidence interval,
IM, intramuscular;
LNP, lipid nanoparticle;
mRNA, messenger RNA.

Intranasal mRNA-LNP Vaccination Limits Viral Replication in the Respiratory Tract and Protects Against Disease

Three weeks after the second dose (Day 42), all vaccinated and mock-vaccinated hamsters were challenged intranasally with 105 plaque-forming units (PFU) of SARS-CoV-2 (isolate USA-WA1/2020; FIG. 20). This isolate was selected for challenge as ancestral SARS-CoV-2 isolates are more pathogenic and drive more severe disease in hamsters than omicron lineage viruses.³³ Viral loads in nasal turbinates and lung were then assessed 3 days (Day 45; n=5 animals per group) and 14 days (Day 56; n=5 animals per group) after challenge. Body weight was evaluated daily.

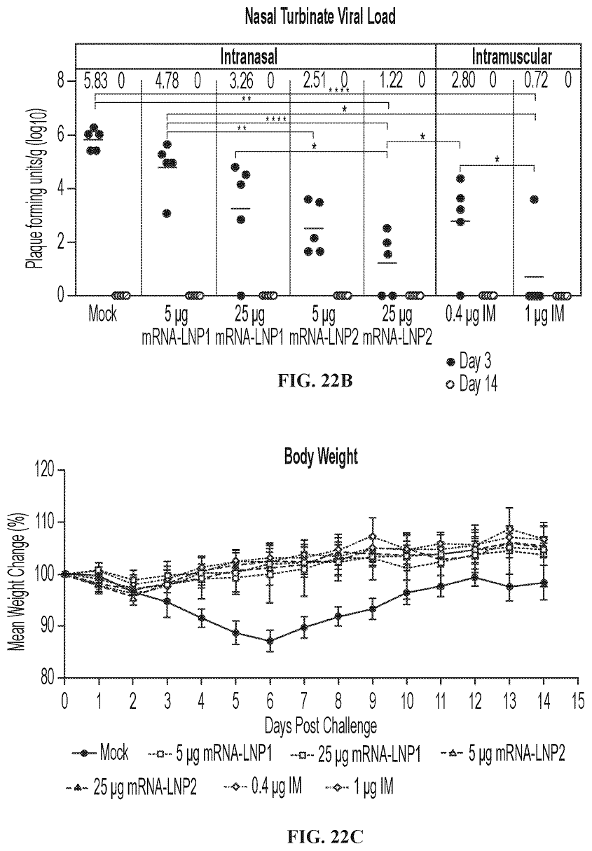

At 3 days after SARS-CoV-2 challenge, intranasally vaccinated hamsters had lower viral loads in both the lung and nasal turbinates relative to mock-vaccination, as determined by plaque assay (FIGS. 22A and 22B, respectively). In the lung, viral loads were below the levels of detection in 4 of 5 hamsters vaccinated with mRNA-LNP2 25 μg, which was significantly reduced relative to mock-vaccinated controls (5 of 5 animals; P<0.05; Table 31). Viral loads were detected in 3 hamsters vaccinated with mRNA-LNP2 5 μg or mRNA-LNP1 25 μg, similar to the 0.4-μg intramuscular vaccine dose level (3 of 5 hamsters). At the 1 μg intramuscular dose level, which is considered protective in this hamster model,³⁴ 2 hamsters had detectable viral loads, which was significantly reduced compared with mock-vaccinated controls (P<0.05). Overall, viral loads in the lung were lower among animals intranasally administered mRNA-LNP2 than mRNA-LNP1 at the respective dose levels, which was significant at the 5-μg dose level (P<0.05).

TABLE 31
Statistical comparisons of viral load by plaque assay in the lungs of
vaccinated hamsters at 3 days after SARS-CoV-2 challenge

| | Fold-change | t- | P- |
|---|---|---|---|
| Comparison | (standard error) | ratio | value |
| 0.4 μg IM over 1 μg IM | 2.32 | 1.88 | 0.783 |
| | (1.23) | | |
| 0.4 μg IM over 25 μg mRNA-LNP1 | 1.15 | 0.98 | 1 |
| | (1.17) | | |
| 0.4 μg IM over 5 μg mRNA-LNP1 | −1.91 | −1.72 | 0.881 |
| | (1.11) | | |
| 0.4 μg IM over 25 μg mRNA-LNP2 | 2.77 | 2.64 | 0.245 |
| | (1.05) | | |
| 0.4 μg IM over 5 μg mRNA-LNP2 | 0.99 | 1.01 | 1 |
| | (0.98) | | |
| 0.4 μg IM over mock | −2.60 | −2.88 | 0.148 |
| | (0.90) | | |
| 1 μg IM over 25 μg mRNA-LNP1 | 0.01 | 0.01 | 1 |
| | (1.23) | | |
| 1 μg IM over 5 μg mRNA-LNP1 | −3.05 | −2.60 | 0.266 |
| | (1.17) | | |
| 1 μg IM over 25 μg mRNA-LNP2 | 1.63 | 1.46 | 0.97 |
| | (1.11) | | |
| 1 μg IM over 5 μg mRNA-LNP2 | −0.15 | −0.15 | 1 |
| | (1.05) | | |
| 1 μg IM over mock | −3.73 | −3.82 | 0.014 |
| | (0.98) | | |
| 25 μg mRNA-LNP1 over 5 μg | −1.88 | −1.53 | 0.955 |
| mRNA-LNP1 | (1.23) | | |
| 25 μg mRNA-LNP1 over 25 μg | 2.79 | 2.39 | 0.401 |
| mRNA-LNP2 | (1.17) | | |
| 25 μg mRNA-LNP1 over 5 μg | 1.02 | 0.91 | 1 |
| mRNA-LNP2 | (1.11) | | |
| 25 μg mRNA-LNP1 over mock | −2.57 | −2.45 | 0.354 |
| | (1.05) | | |
| 5 μg mRNA-LNP1 over 25 μg | 5.85 | 4.76 | 0.001 |
| mRNA-LNP2 | (1.23) | | |
| 5 μg mRNA-LNP1 over 5 μg | 4.07 | 3.48 | 0.035 |
| mRNA-LNP2 | (1.17) | | |
| 5 μg mRNA-LNP1 over mock | 0.49 | 0.44 | 1 |
| | (1.11) | | |
| 25 μg mRNA-LNP2 over 5 μg | −0.60 | −0.49 | 1 |
| mRNA-LNP2 | (1.23) | | |
| 25 μg mRNA-LNP2 over mock | −4.18 | −3.57 | 0.027 |
| | (1.17) | | |
| 5 μg mRNA-LNP2 over mock | −2.40 | −1.96 | 0.731 |
| | (1.23) | | |

ªViral loads (log₁₀ transformed) were assessed by ordinary linear regression;
only modeled data at Day 3 after challenge was evaluated since viral loads on Day 14 were zero for all hamsters.
A degree of freedom of 28 was used.
IM, intramuscular;
LNP, lipid nanoparticle;
mRNA, messenger RNA.

Similarly, in nasal turbinates, viral loads were undetected in 1 of 5 hamsters vaccinated with the 25-μg dose level of mRNA-LNP1 and 2 of 5 hamsters vaccinated with the 25-μg dose level mRNA-LNP2; loads were significantly lower with mRNA-LNP2 (25 μg) than mock-vaccination (P<0.0; Table 32). Viral titers among hamsters intranasally vaccinated with the 5-μg dose level of either intranasal composition remained detectable at 3 days after infection, but titers were numerically lower relative to mock-vaccination and were generally similar to intramuscular vaccination (0.4 μg). Overall, viral reduction in the lungs and nasal turbinates of groups intranasally vaccinated with either dose level of mRNA-LNP2 was comparable to intramuscularly vaccinated groups at the respective dose level. By 14 days after challenge, SARS-CoV-2 virus was not detectable in lung or nasal turbinates of any intranasally or intramuscularly vaccinated hamsters, including mock-vaccinated hamsters (FIGS. 22A and 22B).

TABLE 32
Statistical comparisons of viral load by plaque assay in the nasal turbinates
of vaccinated hamsters at 3 days after SARS-CoV-2 challenge

| | Fold-change | t- | P- |
|---|---|---|---|
| Comparison | (standard error) | ratio | value |
| 0.4 μg IM over 1 μg IM | 3.09 | 3.56 | 0.028 |
| | (0.87) | | |
| 0.4 μg IM over 25 μg mRNA- | 0.55 | 0.67 | 1 |
| LNP1 | (0.83) | | |
| 0.4 μg IM over 5 μg mRNA-LNP1 | −0.97 | −1.24 | 0.995 |
| | (0.78) | | |
| 0.4 μg IM over 25 μg mRNA- | 2.59 | 3.52 | 0.031 |
| LNP2 | (0.74) | | |
| 0.4 μg IM over 5 μg mRNA-LNP2 | 1.30 | 1.89 | 0.779 |
| | (0.69) | | |
| 0.4 μg IM over mock | −2.02 | −3.18 | 0.073 |
| | (0.64) | | |
| 1 μg IM over 25 μg mRNA-LNP1 | −1.53 | −1.76 | 0.86 |
| | (0.87) | | |
| 1 μg IM over 5 μg mRNA-LNP1 | −3.05 | −3.69 | 0.02 |
| | (0.83) | | |
| 1 μg IM over 25 μg mRNA-LNP2 | 0.52 | 0.66 | 1 |
| | (0.78) | | |
| 1 μg IM over 5 μg mRNA-LNP2 | −0.78 | −1.05 | 0.999 |
| | (0.74) | | |
| 1 μg IM over mock | −4.09 | −5.95 | 0 |
| | (0.69) | | |
| 25 μg mRNA-LNP1 over 5 μg | −0.51 | −0.59 | 1 |
| mRNA-LNP1 | (0.87) | | |
| 25 μg mRNA-LNP1 over 25 μg | 3.06 | 3.70 | 0.019 |
| mRNA-LNP2 | (0.83) | | |
| 25 μg mRNA-LNP1 over 5 μg | 1.76 | 2.25 | 0.5 |
| mRNA-LNP2 | (0.78) | | |
| 25 μg mRNA-LNP1 over mock | −1.56 | −2.11 | 0.607 |
| | (0.74) | | |
| 5 μg mRNA-LNP1 over 25 μg | 4.58 | 5.29 | 0 |
| mRNA-LNP2 | (0.87) | | |
| 5 μg mRNA-LNP1 over 5 μg | 3.28 | 3.98 | 0.009 |
| mRNA-LNP2 | (0.83) | | |
| 5 μg mRNA-LNP1 over mock | −0.03 | −0.04 | 1 |
| | (0.78) | | |
| 25 μg mRNA-LNP2 over 5 μg | −0.28 | −0.33 | 1 |
| mRNA-LNP2 | (0.87) | | |
| 25 μg mRNA-LNP2 over mock | −3.60 | −4.36 | 0.003 |
| | (0.83) | | |
| 5 μg mRNA-LNP2 over mock | −2.31 | −2.66 | 0.236 |
| | (0.87) | | |

^{a}Viral loads (log₁₀ transformed) were assessed by ordinary linear regression;
only modeled data at Day 3 after challenge was evaluated since viral loads on Day 14 were zero for all hamsters.
A degree of freedom of 28 was used.
IM, intramuscular;
LNP, lipid nanoparticle;
mRNA, messenger RNA.

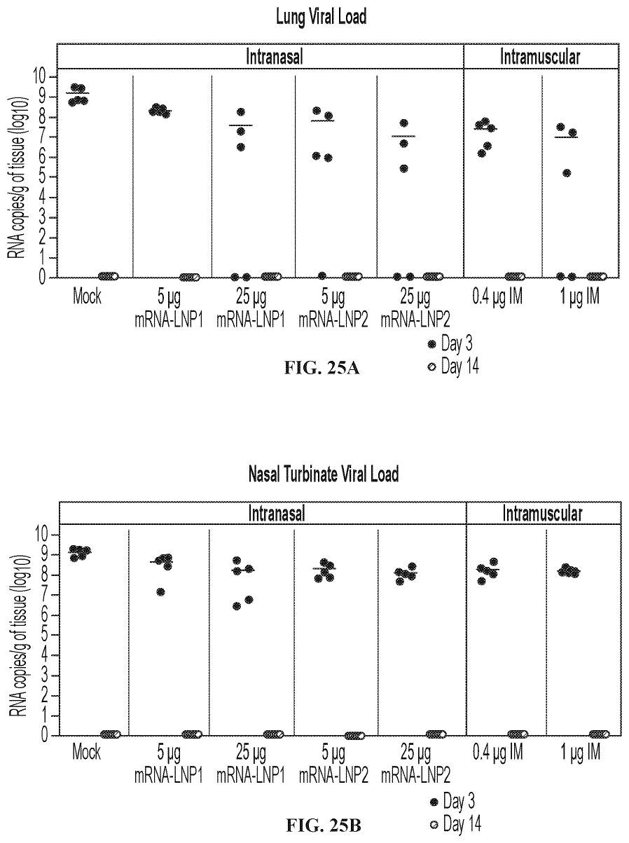

Viral load in respiratory tissues was ailso determined through assessment of viral subgenomic RNA (sgRNA) levels by quantitative reverse transcription polymerase chain reaction (qRT-PCR). Corroborating the plaque assay results, all vaccinated hamsters at 3 days post SARS-CoV-2 challenge had slightly lower viral sgRNA levels relative to mock-vaccinated controls, regardless of dosage and route of administration (FIGS. 25A and 25B). By 14 days after challenge, sgRNA was not detectable in lung or nasal turbinates of any hamsters, including those mock-vaccinated.

SARS-CoV-2 infection was performed with a sub-lethal viral dose known to result in disease characteristics such as weight-loss in Syrian golden hamsters.³⁵ Over the course of infection, mock-vaccinated hamsters experienced a maximum mean (±standard error) weight loss of 12.9% (±1.02) by day 6 post-challenge (FIG. 22C). Comparatively, all intranasally or intramuscularly vaccinated hamsters maintained their bodyweights over the 14-day post-challenge period.

Intranasal mRNA-LNP Vaccination Reduces Severity of Viral Pathology in the Lungs

In the Syrian golden hamster model, SARS-CoV-2 infection with ancestral strains causes severe pathological lesions in lung tissue by 3 days after infection that typically begins to resolve by 10 days post-infection.³⁵ Therefore, to examine the ability of intranasal mRNA-LNP vaccination to reduce lung pathology after infection, histopathological examination of the lower left lobe of the lung of hamsters was performed at 3 days and 14 days after challenge.

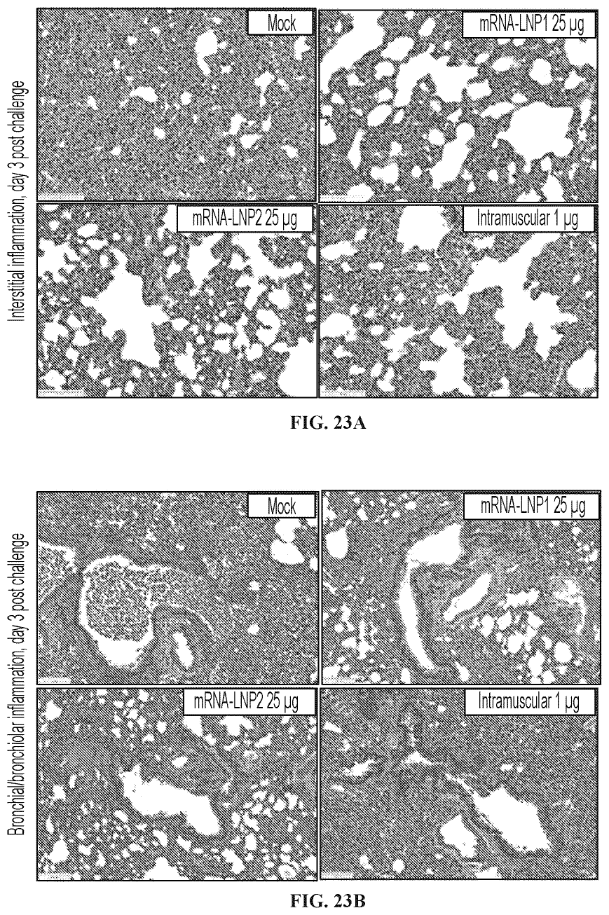

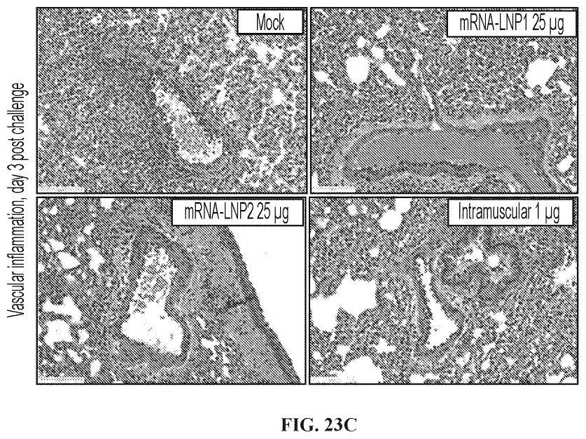

Three days after infection, all vaccinated and mock-vaccinated hamsters exhibited acute pulmonary parenchymal tissue damage and inflammation. There were regionally extensive areas of interstitial infiltration by mixed inflammatory cells, alveolar accumulation of fibrin, hemorrhage, infiltration of bronchial/bronchiolar epithelium by neutrophils, large clusters of intraluminal neutrophils within bronchi/bronchioles with or without epithelial degeneration/necrosis, and vascular inflammation. However, there were marked vaccine group- and dose-dependent differences in severity. Hamsters vaccinated with high dose levels of either intranasal (25 μg) or intramuscular (1 μg) vaccine compositions had similar levels of pulmonary parenchymal inflammation (FIG. 23A) to mock-vaccinated controls, but exhibited lower severity scores for bronchial/bronchiolar inflammation (FIG. 23B; Table 33) and vascular inflammation (FIG. 23C; Table 33). No major difference in lung histopathology were observed for the lower dose levels compared to mock-vaccination (Table 33). Major histopathological findings and severity scores for each group and dose level at 3 days after challenge are summarized in Table 33.

TABLE 33
Major pulmonary histopathological findings and severity scores
after SARS-CoV-2 challenge by vaccine group (Day 3)

| | | Intranasal | | | | | |
|---|---|---|---|---|---|---|---|
| | | mRNA- | mRNA- | mRNA- | mRNA- | | |
| | | LNP1 | LNP1 | LNP2 | LNP2 | Intramuscular | |
| | Mock | 5 μg | 25 μg | 5 μg | 25 μg | 0.4 μg | 1 μg |
| | (n = 5) | (n = 5) | (n = 5) | (n = 5) | (n = 5) | (n = 5) | (n = 5) |
| Interstitial | | | | | | | |
| inflammation, n | | | | | | | |
| Group total | 5 | 5 | 5 | 5 | 5 | 5 | 5 |
| 2 (mild) | 2 | 4 | 2 | 2 | 1 | 5 | 2 |
| 3 (moderate) | 3 | 1 | 3 | 3 | 4 | 0 | 3 |
| Bronchial/bronchiolar | | | | | | | |
| inflammation, n | | | | | | | |
| Group total | 4 | 5 | 1 | 3 | 1 | 3 | 4 |
| 1 (minimal) | 2 | 1 | 1 | 1 | 0 | 2 | 3 |
| 2 (mild) | 0 | 3 | 0 | 2 | 1 | 1 | 1 |
| 3 (moderate) | 2 | 1 | 0 | 0 | 0 | 0 | 0 |
| Vascular | | | | | | | |
| inflammation, n | | | | | | | |
| Group total | 4 | 3 | 2 | 3 | 2 | 4 | 1 |
| 1 (minimal) | 0 | 1 | 1 | 1 | 1 | 3 | 0 |
| 2 (mild) | 2 | 2 | 1 | 1 | 0 | 1 | 1 |
| 3 (moderate) | 2 | 0 | 0 | 1 | 1 | 0 | 0 |

Fourteen days after SARS-CoV-2 infection, there were still regionally extensive areas of interstitial inflammation for all hamsters regardless of vaccine administration route or dose level. However, fibrin accumulation, hemorrhage, bronchial/bronchiolar inflammation, and vascular inflammation regressed, with evidence of tissue recovery such as type II pneumocyte hyperplasia (Table 34). Nonetheless, compared with mock-vaccination controls, all vaccinated groups exhibited lower severity of pulmonary inflammation irrespective of vaccine group or dose level (Table 34). Histopathology at 14 days after challenge for high dose levels are shown in FIGS. 26A-26C.

TABLE 34
Major pulmonary histopathological findings and severity scores
after SARS-CoV-2 challenge by vaccine group (Day 14)

| | | Intranasal | | | | | |
|---|---|---|---|---|---|---|---|
| | | mRNA- | mRNA- | mRNA- | mRNA- | | |
| | | LNP1 | LNP1 | LNP2 | LNP2 | Intramuscular | |
| | Mock | 5 μg | 25 μg | 5 μg | 25 μg | 0.4 μg | 1 μg |
| | (n = 5) | (n = 5) | (n = 5) | (n = 4) | (n = 4) | (n = 5) | (n = 5) |
| Interstitial | | | | | | | |
| inflammation, n | | | | | | | |
| Group total | 5 | 5 | 5 | 4 | 4 | 5 | 5 |
| 2 (mild) | 0 | 5 | 4 | 0 | 4 | 4 | 3 |
| 3 (moderate) | 5 | 0 | 1 | 4 | 0 | 1 | 2 |
| Type II | | | | | | | |
| pneumocyte | | | | | | | |
| hyperplasia, n | | | | | | | |
| Group total | 5 | 4 | 5 | 4 | 2 | 1 | 5 |
| 1 (minimal) | 0 | 3 | 3 | 1 | 2 | 0 | 0 |
| 2 (mild) | 4 | 1 | 1 | 2 | 0 | 0 | 3 |
| 3 (moderate) | 1 | 0 | 1 | 1 | 0 | 1 | 2 |
| Bronchial/ | | | | | | | |
| bronchiolar | | | | | | | |
| inflammation, n | | | | | | | |
| Group total | 4 | 3 | 2 | 3 | 1 | 1 | 3 |
| 1 (minimal) | 2 | 3 | 2 | 1 | 1 | 1 | 3 |
| 2 (mild) | 2 | 0 | 0 | 2 | 0 | 0 | 0 |
| Vascular | | | | | | | |
| inflammation, n | | | | | | | |
| Group total | 1 | 4 | 4 | 4 | 1 | 5 | 3 |
| 1 (minimal) | 1 | 4 | 4 | 4 | 1 | 5 | 3 |

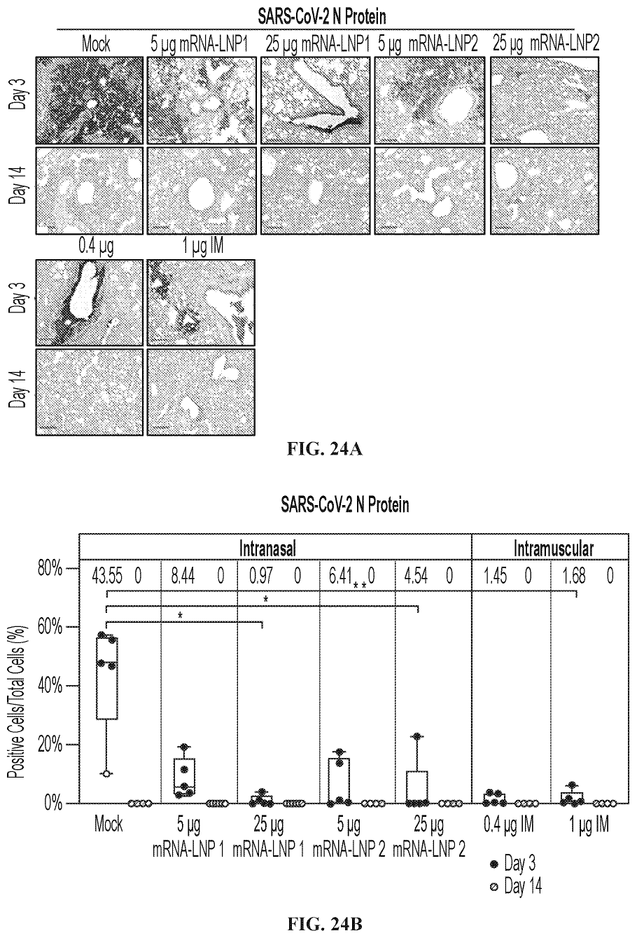

Additionally, lung tissue samples were stained for the SARS-CoV-2 nucleocapsid protein (N protein) by immunohistochemistry to identify cells infected with SARS-CoV-2 (FIGS. 24A-24C). Three days after challenge, all 5 mock-vaccinated hamsters had N-protein+ cells (group mean: 43.55%±19.23% positive cells of total cells quantified); signal was absent at the corresponding 14 days post-challenge, suggesting antibody efficacy in target binding (FIG. 24B). While all mock-vaccinated and vaccinated groups had hamsters positive for N-protein in the lung tissue (Table 35), the vaccinated groups had a lower percentage of N-protein+ cells compared to the mock-vaccinated controls. In intranasally vaccinated groups, the percentage of positive cells decreased in a dose-dependent manner (mRNA-LNP1: 5 μg, 8.44±6.90%; 25 μg, 0.97±1.64%; mRNA-LNP2: 5 μg, 6.41% 8.46; 25 μg, 4.54%±10.12%). All high dose level groups had significantly a lower percentage of N-protein+ cells from mock-vaccinated controls at 3 days after challenge, regardless of administration route (mRNA-LNP1 25 μg P=0.017; mRNA-LNP2 25 μg, P=0.033; intramuscular 1 μg, P=0.004). Percentage of N-protein+ cells for lower dose level groups were not significantly different from mock-vaccinated controls at either 3 or 14 days after challenge. Of note, the mRNA-LNP2 25 μg group had 4 of 5 hamsters with <1% N-protein positive cells, with one hamster with N-protein+ cells comparable to mock-vaccinated control, suggesting a potential breakthrough infection. Generally, intranasal vaccination at higher dose levels was as effective at preventing SARS-CoV-2 infection as intramuscular controls (1.45%±1.82% and 1.68%±2.71% for 0.4 μg and 1 μg, respectively), while the lower dose levels of intranasal vaccines lowered the severity of infection when compared to mock-vaccination. None of the hamsters were positive for N protein at 14 days after challenge (FIG. 24B).

TABLE 35
Summary-Animals Positive for SARS-CoV-2
Nucleocapsid Protein of N Protein Positive

| | | Ratio of N Protein Positive |
|---|---|---|
| | Administration | Hamsters to Total |
| Vaccine Group | Route* | Population (n/N) |
| Mock | IN | 5/5 |
| mRNA-LNP1 5 μg | IN | 5/5 |
| mRNA-LNP1 25 μg | IN | 2/5 |
| mRNA-LNP2 5 μg | IN | 4/5 |
| mRNA-LNP2 25 μg | IN | 4/5 |
| Intramuscular 0.4 μg | IM | 3/5 |
| Intramuscular 1 μg | IM | 2/5 |

*IN, intranasal;
IM, intramuscular;
LNP, lipid nanoparticle;
mRNA, messenger RNA;
N protein, nucleocapsid protein;
SARS-CoV-2, severe acute respiratory syndrome coronavirus 2.

### Discussion

Intranasal vaccine regimens may establish local immunity at upper respiratory sites and act as an early, protective barrier to reduce viral infection and subsequent transmission;^{7,8,12,18} however, vaccine development for intranasal administration is challenging. The respiratory tract is protected by a slightly acidic mucosal layer containing proteolytic enzymes that form a barrier over the epithelial cell lining that undergoes continuous mucosal clearance.⁸ These mechanisms act to defend against entry of respiratory pathogens but can subsequently prohibit antigen delivery during intranasal vaccination.⁸

An mRNA-LNP-based approach for intranasal vaccination to respiratory pathogens, including SARS-CoV-2, may offer additional advantages over more traditional vaccine development platforms.^{7,16} For example, mRNA-encoded antigens more closely resemble the structure and presentation of viral proteins expressed during a natural infection.⁴² Additionally, an mRNA-based approach uses a single vaccine platform across different pathogens,⁴² with this platform enabling flexible antigen design, inclusion of multiple or modified antigens, and rapid incorporation of sequence substitutions that may be needed due to the emergence of variants.⁴² mRNA-based vaccines may also minimize safety concerns associated with more traditional approaches utilized for mucosal vaccines, including those reliant on a live attenuated virus that have a theoretical risk of reverting to its pathogenic form. In addition, mRNA vaccines have a vector-less approach and thus can avoid the potential for diminished immunogenicity with repeat dosing sometimes observed with vector-based vaccines. Moreover, the utility of intramuscularly administered mRNA vaccines against respiratory pathogens such as SARS-CoV-2 has been established, demonstrating robust immune responses and high real-world effectiveness against disease.^{22,43,44}

This Example demonstrates the immunogenicity and protective efficacy of intranasally administered mRNA-LNP vaccines using SARS-CoV-2 as a model pathogen. Overall, intranasal vaccination elicited systemic immune responses while resulting in lower SARS-CoV-2 infection levels and severity of infection versus mock-vaccinated controls after viral challenge. In particular, two doses of mRNA-LNP2 elicited systemic immune responses that were generally similar to intramuscular administration (0.4 μg and 1.0 μg). Further, vaccination with mRNA-LNP2 led to lower post-challenge viral titers in the lung and nasal turbinates relative to mRNA-LNP1 at the respective 5 μg and 25 μg dose levels, suggesting improved protection against SARS-CoV-2. Both intranasal vaccine formulations at the 25 μg dose level prevented severe lung pathology and reduced SARS-CoV-2 infection within the lungs to a similar degree as intramuscular vaccination. Taken together, these findings indicate that intranasal vaccination with an mRNA-LNP SARS-CoV-2 vaccine is protective and can induce systemic immune responses similar to intramuscular vaccination, which has already been shown to be highly effective against COVID-19.^{21,22,45}

Therefore, as shown herein, intranasally administered mRNA-LNP vaccines delivered as a primary two-dose regimen to naive hamsters are immunogenic and can protect against SARS-CoV-2 infection. Further, LNPs designed for improved delivery to the respiratory tract were more immunogenic and better protected against infection than traditional LNPs delivered intranasally.

### Methods

### Hamster Studies

Female Syrian golden hamsters (6-7 weeks old; Envigo) were intranasally vaccinated with a SARS-CoV-2 vaccine on a 2-dose schedule with 3 weeks between doses (Day 10 and Day 21; FIG. 20). Hamsters (n=10 per vaccine group) were intranasally administered 40 μL (split between each naris) of SARS-CoV-2 vaccine (5 μg or 25 μg) formulated in 2 different LNP compositions; as a control, 1 group (n=10) was administered tris/sucrose buffer (mock-vaccination) intranasally. An additional 2 groups (n=10 per group) were intramuscularly vaccinated into the hind leg with the SARS-CoV-2 vaccine (0.4 μg or 1 μg), formulated with Compound 25, into the hind leg. Serum samples for immunogenicity assessments were collected at 3 weeks after dose 1 (Day 20) and 3 weeks after dose 2 (Day 41).

At 21 days after dose 2 (Day 42), all vaccinated hamsters were infected with 100 μL (50 μL/naris) SARS-CoV-2 (2019-nCoV/USA-WA1/2020; Genbank: MN985325.1) at 10⁵ PFU. Through 14 days after viral challenge, hamsters were monitored daily for weight changes. At 3 days and 14 days post-infection, lungs and nasal turbinates were collected from each vaccine group (n=5 animals per timepoint). Prior to SARS-CoV-2 challenge, one animal each in the mRNA-LNP2 5-μg and 25-μg 2 groups died, one succumbed to territorial behavior and the other cause of death was unknown.

Preclinical mRNA and Lipid Nanoparticle Production Process

A sequence-optimized mRNA encoding the SARS-CoV-2 S protein with 6 proline mutations³² was in vitro synthesized and oligo-dT affinity purified as previously described.²⁷ mRNA was LNP-encapsulated via nanoprecipitation by microfluidic mixing of ionzizable,^{27,47} structural, helper, and polyethylene glycol lipids in acetate buffer (pH 5.0), followed by buffer exchange, concentration via tangential flow filtration, and filtration through a 0.8/0.2 μm membrane; an additional lipid was added for mRNA-LNP2. The drug product was analytically characterized, and the products were evaluated as acceptable for in vivo use.

### S-2P-Specific ELISA

MaxiSorp 96-well flat-bottom plates (Thermo Fisher Scientific) were coated with 1 μg/mL (for IgG) or 5 μg/mL (for IgA) S-2P protein (GenScript), corresponding to the spike protein of the Wuhan-Hu-1 virus stabilized with 2 proline mutations, and incubated at 4° C. overnight. The plates were then washed 4 times with PBS+0.05% Tween-20 and blocked with SuperBlock buffer in PBS (Thermo Fisher Scientific) for 1.5 hours at 37° C. After washing, 5-fold serial dilutions of serum (assay diluent: PBS+5% goat serum [Gibco]+0.05% Tween-20) was added and incubated for 2 hours at 37° C. (IgG) or overnight (IgA). Plates were washed and bound antibodies were detected with horseradish peroxidase (HRP)-conjugated goat anti-hamster IgG antibodies (1:10,000; Abcam; AB7146) or HRP-conjugated rabbit anti-hamster IgA antibodies (1:5,000; Brookwood Biomedical; sab3003) for 1 hour at 37° C. Plates were washed and bound antibody detected with SureBlue TMB substrate (Kirkegaard & Perry Labs, Inc.). After incubating at room temperature in the dark for 12 minutes, 3,3,5,5-tetramethylbenzidine stop solution (Kirkegaard & Perry Labs, Inc.) was added and absorbance was measured at 450 nM. GraphPad Prism (V 9.4.0) was used to determine titers using a 4-parameter logistic curve fit for IgG or defined as the reciprocal dilution at approximately optical density (OD) for IgA with baseline defined as 3-fold above the OD of the blank.

### SARS-CoV-2 Neutralization Assay

Two-fold dilutions of serum (heat inactivated, at an initial 1:10 dilution) were prepared in serum-free minimal essential media (MEM), then incubated with SARS-CoV-2 (2019-nCoV/USA-WA01/2020 at a final concentration of 100 PFU) at 37° C. for 1 hour. Mixtures of virus-sera were then absorbed onto monolayers of Vero-E6 cells for 1 hour at 37° C. in 96-well plates, then replaced with an overlay of MEM/methylcellulose/2% fetal bovine serum (FBS) and incubated for 2 days at 37° C. in humidified 5% CO₂. Plaques were immunostained as described below for viral load analysis by plaque assay and then counted with the ImmunoSpot analyzer (CTL); neutralization titers were determined at an endpoint of 60% plaque reduction.

### Analysis of Viral Load by Plaque Assay

Nasal turbinates and right lung were homogenized in Leibovitz L-15 medium (Thermo Fisher Scientific) supplemented with 10% FBS and 1× antibiotic-antimycotic by a TissueLyser II bead mill with 5-mm stainless steel beads (Qiagen). After brief centrifugation, 10-fold serial dilutions of homogenates were prepared in serum-free MEM, then absorbed on 48-well plates of Vero-E6 monolayers for 1 hour at 37° C. The virus inoculum was removed, replaced with an overlay of MEM/methylcellulose/2% FBS, and incubated for 3 days. Plaques were then immunostained using a human monoclonal antibody cocktail specific for the SARS-CoV-2 S protein (Clones DB_A03-09, 12; DB_B01-04, B07-10, 12; DB_C01-05, 07, 09, 10; DB_D01, 02; DB_E01-04, 06, 07; DB_F02-03; Distributed Bio) and an anti-human IgG HRP-conjugated secondary antibody (Cat No. 5220-0456; Sera Care) and then counted to determine virus load per gram of tissue.

Analysis of Viral Load by qRT-PCR

Replicating viral RNA in lung and nasal turbinates was determined via qRT-PCR measuring subgenomic SARS-CoV-2 E gene RNA using previously described primers, probe, and cycle conditions.⁴⁸ In brief, RNA was extracted from homogenates using TRIzol LS (Thermo Fisher Scientific) and Direct-zol RNA Microprep kit (Zymo Research). Quantitative one-step real-time PCR was performed using extracted RNA (10 ng), TaqMan Fast Virus 1-step Master Mix (Thermo Fisher Scientific), primers, and a FAM-ZEN/Iowa Black FQ labeled probe sequence (Integrated DNA Technologies) on the QuantStudio 6 system (Applied Biosystems). An Ultramer DNA oligonucleotide spanning the amplicon (Integrated DNA Technologies) was used for standard curve generation to calculate subgenomic RNA copies per gram of tissue.

### Histopathology

Histological analysis of lung samples proceeded as follows. The lower left lobe of the lung was fixed in 10% neutral buffered formalin, paraffin-embedded, sectioned (5 m), and stained with hematoxylin and eosin (H&E). Sections were evaluated in a blinded manner by a board-certified veterinary pathologist under light microscopy with an Olympus BX51 microscope. Slides were scanned with a 20× (N.A. 0.8) objective at a single layer with continuous stage movement scanning method and images were captured using a Pannoramic 250 Flash III (3DHISTECH). Glass slides were examined, and microscopic diagnoses were graded independently on a 5-level severity scale (grades 1 to 5: minimal, mild, moderate, marked, and severe) by 2 veterinary pathologists.

### Immunohistochemistry

Immunohistochemistry (IHC) was performed on Formalin-Fixed Paraffin Embedded (FFPE) sections using the Lecia Bond RX auto-stainer (Lecia Microsystems). Sections were baked for one hour prior to staining and dewaxed on the instrument. Antigen retrieval was then performed for 20 minutes at 95° C. using Lecia Epitope Retrieval Buffer 2 followed by treatment with Dako serum-free protein block (X090930-2, Agilent Dako) for 15 minutes to prevent non-specific binding of the antibody. Tissue was incubated with 0.083 g/mL of anti-SARS-CoV-2 nucleocapsid protein (GTX135357, GeneTex) for 30 minutes and then detected using Bond Polymer Refine Detection Kit (DS9800, Lecia Microsystems) and bluing reagent (3802918, Lecia Microsystems) to enhance the color. Images were taken at 20× magnification using a Panoramic 250 Flash II scanner (3DHISTECH). Image analysis software was performed using Halo software (Indica Labs).

### Statistical Modeling and Hypothesis Testing

Bayesian linear mixed model was used to model IgG, IgA and neutralization titers, separately. A Bayesian model was chosen for its flexibility in model estimation when the data was censored (left at the limit of detection) and presented heterogeneous group variances. Since the Bayesian model was employed for ease of model fitting, but not as a means to include prior information, we opt for non-informative prior in our analysis. For IgG, IgA, and neutralization titers (log₁₀ titers), each dosing day was modeled separately with one main effect of composition and dose combination (6 levels) and residual variance specific to each dose level (5 μg, 25 μg, 0.4 μg, and 1 μg). Default priors in the brms R package was used, with non-informative flat priors used for all regression coefficients. Holm's method was used to adjust P-values for multiple comparisons. For viral loads (log₁₀ transformed), ordinary linear regression was used with modeled data on Day 3 only as viral loads on Day 14 were zero for all hamsters. Sidak's method was used to adjust P-values for multiple comparisons. All hypothesis testing was done two-sided at alpha level of 0.05, except when noted otherwise. R version 4.1.2 (7) was used for statistical modelling.⁴⁹

Kruskal-Wallis non-parametric test was implemented in hypothesis testing for image analysis using GraphPad's Prism software. This form of ANOVA accounts for the small sample size in each experimental group, as well as the small percentage of N-protein positive cells among animals in the vaccinated groups.

### REFERENCES (EXAMPLE 28)

1 World Health Organization. The top 10 causes of death, <https://www.who.int/news-room/fact-sheets/detail/the-top-10-causes-of-death> (2020).
2 World Health Organization. COVID-19 Vaccine Tracker and Landscape, <https://www.who.int/publications/m/item/draft-landscape-of-covid-19-candidate-vaccines> (2022).
3 Collaborators, G. B. D. L. R. I. Estimates of the global, regional, and national morbidity, mortality, and aetiologies of lower respiratory infections in 195 countries, 1990-2016: a systematic analysis for the Global Burden of Disease Study 2016. Lancet Infect Dis 18, 1191-1210, doi:10.1016/S1473-3099(18)30310-4 (2018).
4 McCloskey, B., Dar, O., Zumla, A. & Heymann, D. L. Emerging infectious diseases and pandemic potential: status quo and reducing risk of global spread. Lancet Infect Dis 14, 1001-1010, doi:10.1016/S1473-3099(14)70846-1 (2014).
5 Centers for Disease, C. & Prevention. Ten great public health achievements—worldwide, 2001-2010. MMWR Morb Mortal Wkly Rep 60, 814-818 (2011).
6 Lavelle, E. C. & Ward, R. W. Mucosal vaccines—fortifying the frontiers. Nat Rev Immunol 22, 236-250, doi:10.1038/s41577-021-00583-2 (2022).
7 Mouro, V. & Fischer, A. Dealing with a mucosal viral pandemic: lessons from COVID-19 vaccines. Mucosal Immunol 15, 584-594, doi:10.1038/s41385-022-00517-8 (2022).
8 Alu, A. et al. Intranasal COVID-19 vaccines: From bench to bed. EBioMedicine 76, 103841, doi:10.1016/j.ebiom.2022.103841 (2022).
9 Yusuf, H. & Kett, V. Current prospects and future challenges for nasal vaccine delivery. Hum Vaccin Immunother 13, 34-45, doi:10.1080/21645515.2016.1239668 (2017).
10 Russell, M. W., Moldoveanu, Z., Ogra, P. L. & Mestecky, J. Mucosal Immunity in COVID-19: A Neglected but Critical Aspect of SARS-CoV-2 Infection. Front Immunol 11, 611337, doi:10.3389/fimmu.2020.611337 (2020).
11 Lapuente, D. et al. Protective mucosal immunity against SARS-CoV-2 after heterologous systemic prime-mucosal boost immunization. Nat Commun 12, 6871, doi:10.1038/s41467-021-27063-4 (2021).
12 Hassan, A. O. et al. A Single-Dose Intranasal ChAd Vaccine Protects Upper and Lower Respiratory Tracts against SARS-CoV-2. Cell 183, 169-184.e113, doi:10.1016/j.cell.2020.08.026 (2020).
13 Hartwell, B. L. et al. Intranasal vaccination with lipid-conjugated immunogens promotes antigen transmucosal uptake to drive mucosal and systemic immunity. Sci Transl Med 14, eabn1413, doi:10.1126/scitranslmed.abn1413 (2022).
14 van Doremalen, N. et al. Intranasal ChAdOx1 nCoV-19/AZD1222 vaccination reduces viral shedding after SARS-CoV-2 D614G challenge in preclinical models. Sci Transl Med 13, doi:10.1126/scitranslmed.abh0755 (2021).
15 Chavda, V. P., Vora, L. K., Pandya, A. K. & Patravale, V. B. Intranasal vaccines for SARS-CoV-2: From challenges to potential in COVID-19 management. Drug Discov Today 26, 2619-2636, doi:10.1016/j.drudis.2021.07.021 (2021).
16 Birkhoff, M., Leitz, M. & Marx, D. Advantages of Intranasal Vaccination and Considerations on Device Selection. Indian Journal of Pharmaceutical Sciences 71, 729-731 (2009).
17 Freeman, D. et al. Injection fears and COVID-19 vaccine hesitancy. Psychol Med, 1-11, doi:10.1017/S0033291721002609 (2021).
18 Krammer, F. SARS-CoV-2 vaccines in development. Nature 586, 516-527, doi:10.1038/s41586-020-2798-3 (2020).
19 Waltz, E. China and India approve nasal COVID vaccines—are they a game changer?Nature 609, 450, doi:10.1038/d41586-022-02851-0 (2022).
20 Package Insert—SPIKEVAX, <https://www.fda.gov/media/155675/download> (2022).
21 Bruxvoort, K. et al. Real-World Effectiveness of the mRNA-1273 Vaccine Against COVID-19: Interim Results from a Prospective Observational Cohort Study. Preprints with THE LANCET, doi:Available at SSRN: https://ssrn.com/abstract=3916094 or http://dx.doi.org/10.2139/ssrn.3916094 (2021).
22 Chemaitelly, H. et al. mRNA-1273 COVID-19 vaccine effectiveness against the B.1.1.7 and B.1.351 variants and severe COVID-19 disease in Qatar. Nat Med, doi:10.1038/s41591-021-01446-y (2021).
23 Dickerman, B. A. et al. Comparative Effectiveness of BNT162b2 and mRNA-1273 Vaccines in U.S. Veterans. New England Journal of Medicine 386, 105-115, doi:10.1056/NEJMoa2115463 (2021).
24 Polack, F. P. et al. Safety and efficacy of the BNT162b2 mRNA Covid-19 vaccine. N Engl J Med 383, 2603-2615, doi:10.1056/NEJMoa2034577 (2020).
25 Pilishvili, T. et al. Effectiveness of mRNA Covid-19 Vaccine among U.S. Health Care Personnel. N Engl J Med 385, e90, doi:10.1056/NEJMoa2106599 (2021).
26 Baden, L. R. et al. Efficacy and safety of the mRNA-1273 SARS-CoV-2 vaccine. N Engl J Med 384, 403-416, doi:10.1056/NEJMoa2035389 (2021).
27 Hassett, K. J. et al. Optimization of Lipid Nanoparticles for Intramuscular Administration of mRNA Vaccines. Mol Ther Nucleic Acids 15, 1-11, doi:10.1016/j.omtn.2019.01.013 (2019).
28 Mendonca, S. A., Lorincz, R., Boucher, P. & Curiel, D. T. Adenoviral vector vaccine platforms in the SARS-CoV-2 pandemic. NPJ Vaccines 6, 97, doi:10.1038/s41541-021-00356-x (2021).
29 Pardi, N., Hogan, M. J., Porter, F. W. & Weissman, D. mRNA vaccines—a new era in vaccinology. Nat Rev Drug Discov 17, 261-279, doi:10.1038/nrd.2017.243 (2018).
30 Veiga, N., Diesendruck, Y. & Peer, D. Targeted lipid nanoparticles for RNA therapeutics and immunomodulation in leukocytes. Adv Drug Deliv Rev 159, 364-376, doi:10.1016/j.addr.2020.04.002 (2020).
31 Tombacz, I. et al. Highly efficient CD4+ T cell targeting and genetic recombination using engineered CD4+ cell-homing mRNA-LNPs. Mol Ther 29, 3293-3304, doi:10.1016/j.ymthe.2021.06.004 (2021).
32 Hsieh, C. L. et al. Structure-based design of prefusion-stabilized SARS-CoV-2 spikes. Science 369, 1501-1505, doi:10.1126/science.abd0826 (2020).
33 Halfmann, P. J. et al. SARS-CoV-2 Omicron virus causes attenuated disease in mice and hamsters. Nature 603, 687-692, doi:10.1038/s41586-022-04441-6 (2022).
34 Meyer, M. et al. Attenuated activation of pulmonary immune cells in mRNA-1273-vaccinated hamsters after SARS-CoV-2 infection. J Clin Invest 131, doi:10.1172/JC1148036 (2021).
35 Imai, M. et al. Syrian hamsters as a small animal model for SARS-CoV-2 infection and countermeasure development. Proc Natl Acad Sci USA 117, 16587-16595, doi:10.1073/pnas.2009799117 (2020).
36 Bharat Biotech International Limited. iNCOVACC®, World's first Intranasal Vaccine to receive both Primary series and Heterologous booster approval, <https://www.bharatbiotech.com/images/press/bharat-biotech-incovacc-booster-approval-press-release.pdf> (2022).
37 CanSino Biologics Inc. CanSinoBIO's Convidecia Air™ Receives Approval in China, <https://www.cansinotech.com/html/1/179/180/1100.html> (2022).
38 Wong, T. Y. et al. Intranasal administration of BReC-CoV-2 COVID-19 vaccine protects K18-hACE2 mice against lethal SARS-CoV-2 challenge. npj Vaccines 7, 36, doi:10.1038/s41541-022-00451-7 (2022).
39 Zhang, Z. et al. Aerosolized Ad5-nCoV booster vaccination elicited potent immune response against the SARS-CoV-2 Omicron variant after inactivated COVID-19 vaccine priming. medRxiv, 2022.2003.2008.22271816, doi:10.1101/2022.03.08.22271816 (2022).
40 Tioni, M. F. et al. One mucosal administration of a live attenuated recombinant COVID-19 vaccine protects nonhuman primates from SARS-CoV-2. bioRxiv, 2021.2007.2016.452733, doi:10.1101/2021.07.16.452733 (2021).
41 Madhavan, M. et al. Tolerability and immunogenicity of an intranasally-administered adenovirus-vectored 60 COVID-19 vaccine: An open-label partially-randomised ascending dose phase I trial. EBioMedicine, 104298, doi:10.1016/j.ebiom.2022.104298 (2022).
42 Edwards, D. K. & Carfi, A. Messenger ribonucleic acid vaccines against infectious diseases: current concepts and future prospects. Curr Opin Immunol 77, 102214, doi:10.1016/j.coi.2022.102214 (2022).
43 Choi, A. et at. Safety and immunogenicity of SARS-CoV-2 variant mRNA vaccine boosters in healthy adults: an interim analysis. Nat Med, doi: 10.1038/s41591-021-01527-y (2021).
44 Bruxvoort, K. J. et al. Real-world effectiveness of the mRNA-1273 vaccine against COVID-19: Interim results from a prospective observational cohort study. Lancet Reg Health Am, 100134, doi:10.1016/j.lana.2021.100134 (2021).
45 El Sahly, H. M. et al. Efficacy of the mRNA-1273 SARS-CoV-2 Vaccine at Completion of Blinded Phase. N Engl J Med 385, 1774-1785, doi:10.1056/NEJMoa213017 (2021).
46 Chan, J. F. et al. Simulation of the Clinical and Pathological Manifestations of Coronavirus Disease 2019 (COVID-19) in a Golden Syrian Hamster Model: Implications for Disease Pathogenesis and Transmissibility. Clin Infect Dis 71, 2428-2446, doi: 10.1093/cid/ciaa325 (2020).
47 Sabnis, S. et al. A Novel Amino Lipid Series for mRNA Delivery: Improved Endosomal Escape and Sustained Pharmacology and Safety in Non-human Primates. Mol Ther 26, 1509-1519, doi:10.1016/j.ymthe.2018.03.010 (2018).
48 Wolfel, R. et al. Virological assessment of hospitalized patients with COVID-2019. Nature 581, 465-469, doi: 10.1038/s41586-020-2196-x (2020).
49 R Core Team. R: A language and environment for statistical computing. R Foundation for Statistical Computing, Vienna, Austria. URL https://www.R-project.org/. 2021).

TABLE 36
Sequences
HexaPro (Examples 21 and 28)

| SEQ ID NO: 1 consists of from 5′ end to 3′ end: 5′ UTR SEQ ID NO: 2, mRNA | | SEQ ID |
|---|---|---|
| ORF SEQ ID NO: 3, and 3′ UTR SEQ ID NO: 4. | | NO: 1 |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCGGC | SEQ ID |
| | GCCGCCACC | NO: 2 |
| ORF of mRNA | AUGUUCGUGUUCCUGGUGCUGCUGCCCCUGGUGAGCAGCCAGUGCGUG | SEQ ID |
| (excluding the stop | AACCUGACCACCCGGACCCAGCUGCCACCAGCCUACACCAACAGCUUC | NO: 3 |
| codon) | ACCCGGGGCGUCUACUACCCCGACAAGGUGUUCCGGAGCAGCGUCCUG | |
| | CACAGCACCCAGGACCUGUUCCUGCCCUUCUUCAGCAACGUGACCUGG | |
| | UUCCACGCCAUCCACGUGAGCGGCACCAACGGCACCAAGCGGUUCGAC | |
| | AACCCCGUGCUGCCCUUCAACGACGGCGUGUACUUCGCCAGCACCGAG | |
| | AAGAGCAACAUCAUCCGGGGCUGGAUCUUCGGCACCACCCUGGACAGC | |
| | AAGACCCAGAGCCUGCUGAUCGUGAAUAACGCCACCAACGUGGUGAUC | |
| | AAGGUGUGCGAGUUCCAGUUCUGCAACGACCCCUUCCUGGGCGUGUAC | |
| | UACCACAAGAACAACAAGAGCUGGAUGGAGAGCGAGUUCCGGGUGUAC | |
| | AGCAGCGCCAACAACUGCACCUUCGAGUACGUGAGCCAGCCCUUCCUG | |
| | AUGGACCUGGAGGGCAAGCAGGGCAACUUCAAGAACCUGCGGGAGUUC | |
| | GUGUUCAAGAACAUCGACGGCUACUUCAAGAUCUACAGCAAGCACACC | |
| | CCAAUCAACCUGGUGCGGGAUCUGCCCCAGGGCUUCUCAGCCCUGGAG | |
| | CCCCUGGUGGACCUGCCCAUCGGCAUCAACAUCACCCGGUUCCAGACC | |
| | CUGCUGGCCCUGCACCGGAGCUACCUGACCCCAGGCGACAGCAGCAGC | |
| | GGGUGGACAGCAGGCGCGGCUGCUUACUACGUGGGCUACCUGCAGCCC | |
| | CGGACCUUCCUGCUGAAGUACAACGAGAACGGCACCAUCACCGACGCC | |
| | GUGGACUGCGCCCUGGACCCUCUGAGCGAGACCAAGUGCACCCUGAAG | |
| | AGCUUCACCGUGGAGAAGGGCAUCUACCAGACCAGCAACUUCCGGGUG | |
| | CAGCCCACCGAGAGCAUCGUGCGGUUCCCCAACAUCACCAACCUGUGC | |
| | CCCUUCGGCGAGGUGUUCAACGCCACCCGGUUCGCCAGCGUGUACGCC | |
| | UGGAACCGGAAGCGGAUCAGCAACUGCGUGGCCGACUACAGCGUGCUG | |
| | UACAACAGCGCCAGCUUCAGCACCUUCAAGUGCUACGGCGUGAGCCCC | |
| | ACCAAGCUGAACGACCUGUGCUUCACCAACGUGUACGCCGACAGCUUC | |
| | GUGAUCCGUGGCGACGAGGUGCGGCAGAUCGCACCCGGCCAGACAGGC | |
| | AAGAUCGCCGACUACAACUACAAGCUGCCCGACGACUUCACCGGCUGC | |
| | GUGAUCGCCUGGAACAGCAACAACCUCGACAGCAAGGUGGGCGGCAAC | |
| | UACAACUACCUGUACCGGCUGUUCCGGAAGAGCAACCUGAAGCCCUUC | |
| | GAGCGGGACAUCAGCACCGAGAUCUACCAAGCCGGCUCCACCCCUUGC | |
| | AACGGCGUGGAGGGCUUCAACUGCUACUUCCCUCUGCAGAGCUACGGC | |
| | UUCCAGCCCACCAACGGCGUGGGCUACCAGCCCUACCGGGUGGUGGUG | |
| | CUGAGCUUCGAGCUGCUGCACGCCCCAGCCACCGUGUGUGGCCCCAAG | |
| | AAGAGCACCAACCUGGUGAAGAACAAGUGCGUGAACUUCAACUUCAAC | |
| | GGCCUUACCGGCACCGGCGUGCUGACCGAGAGCAACAAGAAAUUCCUG | |
| | CCCUUUCAGCAGUUCGGCCGGGACAUCGCCGACACCACCGACGCUGUG | |
| | CGGGAUCCCCAGACCCUGGAGAUCCUGGACAUCACCCCUUGCAGCUUC | |
| | GGCGGCGUGAGCGUGAUCACCCCAGGCACCAACACCAGCAACCAGGUG | |
| | GCCGUGCUGUACCAGGACGUGAACUGCACCGAGGUGCCCGUGGCCAUC | |
| | CACGCCGACCAGCUGACACCCACCUGGCGGGUCUACAGCACCGGCAGC | |
| | AACGUGUUCCAGACCCGGGCCGGUUGCCUGAUCGGCGCCGAGCACGUG | |
| | AACAACAGCUACGAGUGCGACAUCCCCAUCGGCGCCGGCAUCUGUGCC | |
| | AGCUACCAGACCCAGACCAAUUCACCCGGCAGCGGCGGCAGCGUGGCC | |
| | AGCCAGAGCAUCAUCGCCUACACCAUGAGCCUGGGCGCCGAGAACAGC | |
| | GUGGCCUACAGCAACAACAGCAUCGCCAUCCCCACCAACUUCACCAUC | |
| | AGCGUGACCACCGAGAUUCUGCCCGUGAGCAUGACCAAGACCAGCGUG | |
| | GACUGCACCAUGUACAUCUGCGGCGACAGCACCGAGUGCAGCAACCUG | |
| | CUGCUGCAGUACGGCAGCUUCUGCACCCAGCUGAACCGGGCCCUGACC | |
| | GGCAUCGCCGUGGAGCAGGACAAGAACACCCAGGAGGUGUUCGCCCAG | |
| | GUGAAGCAGAUCUACAAGACCCCUCCCAUCAAGGACUUCGGCGGCUUC | |
| | AACUUCAGCCAGAUCCUGCCCGACCCCAGCAAGCCCAGCAAGCGGAGC | |
| | CCCAUCGAGGACCUGCUGUUCAACAAGGUGACCCUAGCCGACGCCGGC | |
| | UUCAUCAAGCAGUACGGCGACUGCCUCGGCGACAUAGCCGCCCGGGAC | |
| | CUGAUCUGCGCCCAGAAGUUCAACGGCCUGACCGUGCUGCCUCCCCUG | |
| | CUGACCGACGAGAUGAUCGCCCAGUACACCAGCGCCCUGUUAGCCGGA | |
| | ACCAUCACCAGCGGCUGGACUUUCGGCGCUGGACCCGCUCUGCAGAUC | |
| | CCCUUCCCCAUGCAGAUGGCCUACCGGUUCAACGGCAUCGGCGUGACC | |
| | CAGAACGUGCUGUACGAGAACCAGAAGCUGAUCGCCAACCAGUUCAAC | |
| | AGCGCCAUCGGCAAGAUCCAGGACAGCCUGAGCAGCACCCCCAGCGCC | |
| | CUGGGCAAGCUGCAGGACGUGGUGAACCAGAACGCCCAGGCCCUGAAC | |
| | ACCCUGGUGAAGCAGCUGAGCAGCAACUUCGGCGCCAUCAGCAGCGUG | |
| | CUGAACGACAUCCUGAGCCGGCUGGACCCUCCCGAGGCCGAGGUGCAG | |
| | AUCGACCGGCUGAUCACUGGCCGGCUGCAGAGCCUGCAGACCUACGUG | |
| | ACCCAGCAGCUGAUCCGGGCCGCCGAGAUUCGGGCCAGCGCCAACCUG | |
| | GCCGCCACCAAGAUGAGCGAGUGCGUGCUGGGCCAGAGCAAGCGGGUG | |
| | GACUUCUGCGGCAAGGGCUACCACCUGAUGAGCUUUCCCCAGAGCGCA | |
| | CCCCACGGAGUGGUGUUCCUGCACGUGACCUACGUGCCCGCCCAGGAG | |
| | AAGAACUUCACCACCGCCCCAGCCAUCUGCCACGACGGCAAGGCCCAC | |
| | UUUCCCCGGGAGGGCGUGUUCGUGAGCAACGGCACCCACUGGUUCGUG | |
| | ACCCAGCGGAACUUCUACGAGCCCCAGAUCAUCACCACCGACAACACC | |
| | UUCGUGAGCGGCAACUGCGACGUGGUGAUCGGCAUCGUGAACAACACC | |
| | GUGUACGAUCCCCUGCAGCCCGAGCUGGACAGCUUCAAGGAGGAGCUG | |
| | GACAAGUACUUCAAGAAUCACACCAGCCCCGACGUGGACCUGGGCGAC | |
| | AUCAGCGGCAUCAACGCCAGCGUGGUGAACAUCCAGAAGGAGAUCGAU | |
| | CGGCUGAACGAGGUGGCCAAGAACCUGAACGAGAGCCUGAUCGACCUG | |
| | CAGGAGCUGGGCAAGUACGAGCAGUACAUCAAGUGGCCCUGGUACAUC | |
| | UGGCUGGGCUUCAUCGCCGGCCUGAUCGCCAUCGUGAUGGUGACCAUC | |
| | AUGCUGUGCUGCAUGACCAGCUGCUGCAGCUGCCUGAAGGGCUGUUGC | |
| | AGCUGCGGCAGCUGCUGCAAGUUCGACGAGGACGACAGCGAGCCCGUG | |
| | CUGAAGGGCGUGAAGCUGCACUACACC | |
| 3′ UTR | UGAUAAUAGGCUGGAGCCUCGGUGGCCUAGCUUCUUGCCCCUUGGGCC | SEQ ID |
| | UCCCCCCAGCCCCUCCUCCCCUUCCUGCACCCGUACCCCCGUGGUCUU | NO: 4 |
| | UGAAUAAAGUCUGAGUGGGCGGC | |
| Corresponding amino acid | MFVFLVLLPLVSSQCVNLTTRTQLPPAYTNSFTRGVYYPDKVFRSSVL | SEQ ID |
| sequence | HSTQDLFLPFFSNVTWFHAIHVSGTNGTKRFDNPVLPFNDGVYFASTE | NO: 5 |
| | KSNIIRGWIFGTTLDSKTQSLLIVNNATNVVIKVCEFQFCNDPFLGVY | |
| | YHKNNKSWMESEFRVYSSANNCTFEYVSQPFLMDLEGKQGNFKNLREF | |
| | VFKNIDGYFKIYSKHTPINLVRDLPQGFSALEPLVDLPIGINITRFQT | |
| | LLALHRSYLTPGDSSSGWTAGAAAYYVGYLQPRTFLLKYNENGTITDA | |
| | VDCALDPLSETKCTLKSFTVEKGIYQTSNFRVQPTESIVRFPNITNLC | |
| | PFGEVFNATRFASVYAWNRKRISNCVADYSVLYNSASFSTFKCYGVSP | |
| | TKLNDLCFTNVYADSFVIRGDEVRQIAPGQTGKIADYNYKLPDDFTGC | |
| | VIAWNSNNLDSKVGGNYNYLYRLFRKSNLKPFERDISTEIYQAGSTPC | |
| | NGVEGFNCYFPLQSYGFQPTNGVGYQPYRVVVLSFELLHAPATVCGPK | |
| | KSTNLVKNKCVNFNFNGLTGTGVLTESNKKELPFQQFGRDIADTTDAV | |
| | RDPQTLEILDITPCSFGGVSVITPGTNTSNQVAVLYQDVNCTEVPVAI | |
| | HADQLTPTWRVYSTGSNVFQTRAGCLIGAEHVNNSYECDIPIGAGICA | |
| | SYQTQTNSPGSGGSVASQSIIAYTMSLGAENSVAYSNNSIAIPTNFTI | |
| | SVTTEILPVSMTKTSVDCTMYICGDSTECSNLLLQYGSFCTQLNRALT | |
| | GIAVEQDKNTQEVFAQVKQIYKTPPIKDFGGFNFSQILPDPSKPSKRS | |
| | PIEDLLFNKVTLADAGFIKQYGDCLGDIAARDLICAQKFNGLTVLPPL | |
| | LTDEMIAQYTSALLAGTITSGWTFGAGPALQIPFPMQMAYRFNGIGVT | |
| | QNVLYENQKLIANQFNSAIGKIQDSLSSTPSALGKLQDVVNQNAQALN | |
| | TLVKQLSSNFGAISSVLNDILSRLDPPEAEVQIDRLITGRLQSLQTYV | |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKWPWYIWLGFIAGLIAIVMVTIMLCCMTSCCSCLKGCC | |
| | SCGSCCKFDEDDSEPVLKGVKLHYT | |
| PolyA tail | 100 nt | |

HSV gB Protein (Example 22)

| SEQ ID NO: 6 consists of from 5′ end to 3′ end: 5′ UTR SEQ ID NO: 7, mRNA | | SEQ ID |
|---|---|---|
| ORF SEQ ID NO: 8, and 3′ UTR SEQ ID NO: 4. | | NO: 6 |
| Chemistry | 1-methylpseudouridine | |
| Cap | 7mG(5′)ppp(5′)NlmpNp | |
| 5′ UTR | GAGGAAAUAAGAGAGAAAAGAAGAGUAAGAAGAAAUAUAAGACCCCGG | SEQ ID |
| | CGCCGCCACC | NO: 7 |
| ORF of mRNA | AUGCGGGGUGGCGGACUGGUGUGCGCCCUGGUGGUCGGUGCACUUGUG | SEQ ID |
| (excluding the stop | GCCGCUGUGGCUAGCGCUGCACCAGCCGCACCCAGAGCCAGCGGAGGU | NO: 8 |
| codon) | GUGGCCGCGACCGUUGCAGCCAACGGAGGCCCGGCUUCUCAGCCUCCU | |
| | CCUGUGCCCAGCCCCGCUACCACCAAGGCCCGUAAGCGGAAGACCAAG | |
| | AAGCCUCCCAAGCGGCCCGAGGCUACACCACCACCCGACGCCAACGCA | |
| | ACUGUUGCUGCUGGCCACGCCACCCUGAGGGCCCACCUGCGGGAGAUC | |
| | AAGGUGGAGAACGCCGACGCCCAGUUCUACGUGUGUCCACCUCCAACC | |
| | GGCGCCACCGUGGUACAGUUCGAGCAACCUCGCCGGUGCCCCACUCGA | |
| | CCCGAGGGCCAGAACUACACCGAGGGCAUCGCCGUGGUGUUCAAGGAG | |
| | AACAUCGCCCCAUACAAGUUCAAGGCCACCAUGUACUACAAGGACGUG | |
| | ACCGUGAGCCAGGUGUGGUUCGGCCACCGGUACAGCCAGUUCAUGGGC | |
| | AUCUUCGAGGAUCGGGCCCCAGUGCCCUUCGAGGAGGUGAUCGACAAG | |
| | AUCAACGCCAAGGGCGUGUGCCGGAGCACCGCCAAGUACGUGCGGAAC | |
| | AACAUGGAGACCACCGCCUUCCACCGGGACGACCACGAGACCGACAUG | |
| | GAGCUGAAGCCCGCCAAGGUGGCCACACGGACAAGCCGGGGCUGGCAC | |
| | ACCACCGACCUGAAGUACAACCCAAGCCGCGUGGAGGCGUUCCACCGG | |
| | UACGGCACCACCGUGAACUGCAUCGUGGAGGAGGUGGACGCCCGGAGC | |
| | GUGUACCCCUACGACGAAUUCGUGCUGGCCACCGGCGACUUCGUGUAC | |
| | AUGAGCCCCUUCUACGGCUACCGGGAGGGCAGCCACACCGAGCACACC | |
| | AGCUACGCCGCCGACCGGUUCAAGCAGGUGGACGGCUUCUACGCCCGG | |
| | GACCUGACCACUAAAGCCCGGGCCACCUCACCAACCACCCGGAACCUG | |
| | CUGACCACCCCAAAGUUCACCGUGGCCUGGGACUGGGUGCCCAAGCGA | |
| | CCCGCCGUGUGCACCAUGACCAAGUGGCAGGAAGUGGACGAGAUGCUG | |
| | CGGGCCGAAUACGGCGGCAGCUUCCGGUUCAGCAGCGACGCCAUCAGC | |
| | ACCACCUUCACCACCAACCUGACCCAGUACAGCCUGAGCCGGGUGGAU | |
| | CUGGGCGACUGCAUCGGCAGAGACGCUCGCGAGGCCAUCGACCGGAUG | |
| | UUCGCCCGGAAGUACAACGCCACCCACAUCAAGGUGGGCCAGCCCCAG | |
| | UACUACCUCGCCACCGGCGGCUUCCUGAUCGCCUACCAGCCCUUGCUG | |
| | AGCAACACCCUGGCCGAGCUGUACGUGCGGGAGUACAUGCGGGAGCAG | |
| | GACCGGAAGCCCCGGAACGCCACACCCGCCCCUCUGAGAGAAGCCCCU | |
| | UCCGCCAACGCCAGCGUGGAGCGGAUCAAGACCACCAGCAGCAUCGAG | |
| | UUCGCCCGGCUGCAGUUCACCUACAACCACAUCCAGCGGCACGUGAAC | |
| | GACAUGCUGGGUCGGAUCGCCGUGGCUUGGUGCGAGCUGCAGAACCAC | |
| | GAGCUGACCCUGUGGAACGAGGCCCGGAAGCUGAACCCCAACGCCAUC | |
| | GCCAGCGCUACUGUGGGCCGGAGGGUAAGCGCUCGGAUGCUGGGCGAC | |
| | GUGAUGGCCGUGAGCACGUGUGUGCCCGUGGCCCCAGACAACGUGAUC | |
| | GUGCAGAACAGCAUGCGGGUGAGUAGCCGGCCUGGAACCUGCUACAGC | |
| | CGGCCCCUGGUGUCUUUCCGGUACGAGGACCAGGGUCCCCUGAUCGAG | |
| | GGCCAGCUGGGCGAGAACAACGAGCUGCGGCUGACUCGCGACGCUCUG | |
| | GAGCCCUGCACCGUGGGCCACAGACGGUACUUCAUCUUCGGCGGCGGC | |
| | UACGUGUACUUCGAGGAGUACGCCUACAGCCACCAGCUGAGCCGGGCC | |
| | GACGUGACCACCGUGAGCACCUUCAUCGACCUGAACAUCACCAUGCUG | |
| | GAGGACCACGAGUUCGUGCCCCUGGAGGUGUACACCCGGCACGAGAUC | |
| | AAGGACAGCGGCCUGCUGGAUUACACCGAGGUGCAGCGGCGGAACCAG | |
| | CUGCACGACCUGCGGUUCGCCGACAUCGACACCGUGAUACGGGCCGAC | |
| | GCAAACGCCGCCAUGUUCGCCGGCCUGUGCGCCUUCUUCGAGGGCAUG | |
| | GGCGACCUGGGCAGAGCCGUGGGCAAGGUGGUGAUGGGCGUGGUGGGC | |
| | GGAGUGGUAAGCGCCGUGAGCGGCGUGAGCAGCUUCAUGAGCAACCCC | |
| | UUCGGUGCCCUGGCAGUGGGCCUGCUGGUGCUUGCUGGCCUGGUCGCU | |
| | GCCUUCUUCGCCUUCCGGUACGUGCUACAGCUGCAGCGGAAC | |
| 3′ UTR | UGAUAAUAGGCUGGAGCCUCGGUGGCCUAGCUUCUUGCCCCUUGGGCC | SEQ ID |
| | UCCCCCCAGCCCCUCCUCCCCUUCCUGCACCCGUACCCCCGUGGUCUU | NO: 4 |
| | UGAAUAAAGUCUGAGUGGGCGGC | |
| Corresponding amino acid | MRGGGLVCALVVGALVAAVASAAPAAPRASGGVAATVAANGGPASQPP | SEQ ID |
| sequence | PVPSPATTKARKRKTKKPPKRPEATPPPDANATVAAGHATLRAHLREI | NO: 9 |
| | KVENADAQFYVCPPPTGATVVQFEQPRRCPTRPEGQNYTEGIAVVFKE | |
| | NIAPYKFKATMYYKDVTVSQVWFGHRYSQFMGIFEDRAPVPFEEVIDK | |
| | INAKGVCRSTAKYVRNNMETTAFHRDDHETDMELKPAKVATRTSRGWH | |
| | TTDLKYNPSRVEAFHRYGTTVNCIVEEVDARSVYPYDEFVLATGDFVY | |
| | MSPFYGYREGSHTEHTSYAADRFKQVDGFYARDLITKARATSPTTRNL | |
| | LTTPKFTVAWDWVPKRPAVCTMTKWQEVDEMLRAEYGGSFRFSSDAIS | |
| | TTFTTNLTQYSLSRVDLGDCIGRDAREAIDRMFARKYNATHIKVGQPQ | |
| | YYLATGGFLIAYQPLLSNTLAELYVREYMREQDRKPRNATPAPLREAP | |
| | SANASVERIKTTSSIEFARLQFTYNHIQRHVNDMLGRIAVAWCELQNH | |
| | ELTLWNEARKLNPNAIASATVGRRVSARMLGDVMAVSTCVPVAPDNVI | |
| | VQNSMRVSSRPGTCYSRPLVSFRYEDQGPLIEGQLGENNELRLTRDAL | |
| | EPCTVGHRRYFIFGGGYVYFEEYAYSHQLSRADVITVSTFIDLNITML | |
| | EDHEFVPLEVYTRHEIKDSGLLDYTEVQRRNQLHDLRFADIDTVIRAD | |
| | ANAAMFAGLCAFFEGMGDLGRAVGKVVMGVVGGVVSAVSGVSSFMSNP | |
| | FGALAVGLLVLAGLVAAFFAFRYVLQLQRN | |
| PolyA tail | 100 nt | |

HSV gC Protein (Example 22)

| SEQ ID NO: 10 consists of from 5′ end to 3′ end: 5′ UTR SEQ ID NO: 11, mRNA | | SEQ ID |
|---|---|---|
| ORF SEQ ID NO: 12, and 3′ UTR SEQ ID NO: 13. | | NO: 10 |
| Chemistry | 1-methylpseudouridine | |
| Cap | 7mG(5′)ppp(5′)NlmpNp | |
| 5′ UTR | GAGGAAAUCGCAAAAUUUGCUCUUCGCGUUAGAUUUCUUUUAGUUUUC | SEQ ID |
| | UCGCAACUAGCAAGCUUUUUGUUCUCGCC | NO: 11 |
| ORF of mRNA | AUGGCACUUGGACGCGUCGGUCUGGCUGUCGGCCUGUGGGGCCUGCUG | SEQ ID |
| (excluding the stop | UGGGUGGGCGUGGUGGUGGUGCUGGCUAACGCCAGCCCCGGACGUACC | NO: 12 |
| codon) | AUCACCGUGGGGCCCAGAGGCAACGCCAGUAACGCCGCGCCAUCAGCC | |
| | AGCCCGAGAAACGCUAGUGCUCCCCGGACCACGCCAACUCCACCACAG | |
| | CCCCGGAAGGCCACCAAGAGCAAGGCCAGCACCGCCAAGCCCGCUCCA | |
| | CCUCCCAAGACCGGCCCACCCAAGACCAGCAGCGAGCCCGUGCGGUGC | |
| | AACCGGCACGAUCCUCUGGCCCGGUACGGCUCACGGGUGCAGAUCCGG | |
| | UGCCGGUUCCCCAACAGCACCAGGACCGAGAGCCGGCUGCAGAUCUGG | |
| | CGGUACGCCACCGCCACAGACGCCGAGAUCGGCACCGCCCCAAGCCUG | |
| | GAGGAGGUGAUGGUGAACGUGUCUGCUCCACCUGGCGGCCAGCUGGUG | |
| | UACGACAGCGCACCCAACCGGACCGAUCCCCACGUGAUCUGGGCAGAA | |
| | GGCGCUGGUCCUGGCGCUAGCCCUCGACUGUACAGCGUGGUCGGCCCA | |
| | CUGGGCAGGCAGCGGCUGAUCAUCGAGGAGCUGACCCUGGAGACCCAG | |
| | GGCAUGUACUACUGGGUGUGGGGCCGGACAGAUCGGCCUAGCGCCUAC | |
| | GGGACCUGGGUGCGGGUUCGCGUGUUCCGGCCACCUAGCCUGACCAUC | |
| | CAUCCCCACGCCGUGCUGGAGGGCCAGCCCUUCAAAGCCACCUGUACC | |
| | GCCGCCACCUACUACCCAGGCAACCGGGCCGAGUUCGUGUGGUUCGAG | |
| | GACGGACGGCGCGUGUUCGACCCCGCCCAGAUCCACACCCAGACCCAG | |
| | GAGAACCCCGACGGCUUCAGCACCGUGAGCACGGUGACCAGCGCUGCC | |
| | GUUGGAGGCCAAGGCCCUCCCAGAACCUUCACCUGCCAGCUGACCUGG | |
| | CACCGGGACAGCGUGAGCGCUUCUCGCCGGAACGCCAGCGGAACUGCC | |
| | AGCGUGCUGCCACGGCCCACCAUCACCAUGGAGUUCACCGGCGACCAC | |
| | GCCGUGUGCACAGCCGGCUGCGUACCCGAGGGCGUGACCUUCGCCUGG | |
| | UUCCUGGGCGACGACAGCAGCCCCGCCGAGAAGGUGGCCGUGGCCAGC | |
| | CAGACCAGCUGUGGAAGACCCGGAACCGCCACCAUCCGGAGCACCCUG | |
| | CCCGUGAGCUACGAGCAGACCGAGUACAUCUGUCGGCUGGCCGGCUAC | |
| | CCUGACGGCAUCCCCGUGUUGGAGCACCACGGCAGCCACCAGCCUCCU | |
| | CCUCGGGAUCCCACCGAGCGGCAGGUGAUCAGGGCCGUGGAGGGUGCA | |
| | GGCAUCGGCGUGGCCGUGCUGGUGGCCGUAGUGCUAGCCGGCACCGCC | |
| | GUGGUAUACCUGACC | |
| 3′ UTR | UAAAGCUCCCCGGGGGCCUCGGUGGCCUAGCUUCUUGCCCCUUGGGCC | SEQ ID |
| | UCCCCCCAGCCCCUCCUCCCCUUCCUGCAGGAUUGAGACUACGGGUGG | NO: 13 |
| | UCUUUGAAUAAAGUCUGAGUGGGCGGC | |
| Corresponding amino acid | MALGRVGLAVGLWGLLWVGVVVVLANASPGRTITVGPRGNASNAAPSA | SEQ ID |
| sequence | SPRNASAPRTTPTPPQPRKATKSKASTAKPAPPPKTGPPKTSSEPVRC | NO: 14 |
| | NRHDPLARYGSRVQIRCRFPNSTRTESRLQIWRYATATDAEIGTAPSL | |
| | EEVMVNVSAPPGGQLVYDSAPNRTDPHVIWAEGAGPGASPRLYSVVGP | |
| | LGRQRLIIEELTLETQGMYYWVWGRTDRPSAYGTWVRVRVFRPPSLTI | |
| | HPHAVLEGQPFKATCTAATYYPGNRAEFVWFEDGRRVFDPAQIHTQTQ | |
| | ENPDGFSTVSTVTSAAVGGQGPPRTFTCQLTWHRDSVSASRRNASGTA | |
| | SVLPRPTITMEFTGDHAVCTAGCVPEGVTFAWFLGDDSSPAEKVAVAS | |
| | QTSCGRPGTATIRSTLPVSYEQTEYICRLAGYPDGIPVLEHHGSHQPP | |
| | PRDPTERQVIRAVEGAGIGVAVLVAVVLAGTAVVYLT | |
| PolyA tail | 100 nt | |

HSV gD Protein (Example 22)

| SEQ ID NO: 15 consists of from 5′ end to 3′ end: 5′ UTR SEQ ID NO: 16, mRNA | | SEQ ID |
|---|---|---|
| ORF SEQ ID NO: 17, and 3′ UTR SEQ ID NO: 18. | | NO: 15 |
| Chemistry | 1-methylpseudouridine | |
| Cap | 7mG(5′)ppp(5′)NlmpNp | |
| 5′ UTR | GGGAAAUCGCAAAAUUUGCUCUUCGCGUUAGAUUUCUUUUAGUUUUCU | SEQ ID |
| | CGCAACUAGCAAGCUUUUUGUUCUCGCC | NO: 16 |
| ORF of mRNA | AUGGGUCGGCUGACCAGCGGAGUUGGCACCGCCGCACUCCUGGUGGUG | SEQ ID |
| (excluding the stop | GCCGUAGGCCUGCGGGUGGUGUGCGCCAAGUACGCCCUGGCCGACCCA | NO: 17 |
| codon) | UCCCUGAAGAUGGCCGACCCUAACCGGUUCCGGGGCAAGAAUCUGCCC | |
| | GUGCUGGAUCAGCUGACCGAUCCUCCUGGCGUGAAGCGGGUGUACCAC | |
| | AUCCAGCCCAGCCUGGAGGAUCCCUUCCAGCCACCCUCCAUCCCCAUC | |
| | ACCGUUUACUACGCCGUGCUGGAGAGAGCCUGCCGCAGCGUGCUGCUG | |
| | CACGCUCCAUCCGAGGCGCCCCAGAUCGUGCGGGGCGCAAGCGACGAG | |
| | GCCCGGAAGCACACCUACAACCUGACCAUCGCCUGGUACCGGAUGGGC | |
| | GACAACUGCGCCAUCCCUAUCACCGUGAUGGAGUACACCGAGUGCCCC | |
| | UACAACAAGAGCCUGGGAGUGUGCCCCAUCCGGACCCAGCCUCGGUGG | |
| | UCCUACUACGACAGCUUCAGCGCCGUGUCCGAGGACAACCUGGGCUUC | |
| | CUGAUGCACGCCCCUGCCUUCGAGACCGCCGGCACCUACCUGCGGCUG | |
| | GUGAAGAUCAACGACUGGACCGAGAUCACCCAGUUCAUCCUGGAGCAC | |
| | CGGGCAAGGGCCAGCUGCAAGUACGCGCUGCCUCUGCGGAUCCCUCCC | |
| | GCUGCUUGCCUGACCAGCAAGGCCUACCAGCAGGGCGUGACCGUGGAC | |
| | AGCAUCGGCAUGCUGCCCAGGUUCAUCCCCGAGAACCAGCGCACCGUG | |
| | GCCCUGUACAGCCUGAAGAUCGCAGGCUGGCACGGACCCAAGCCUCCU | |
| | UACACCUCCACCCUGCUGCCACCCGAGCUGAGCGACACCACCAACGCC | |
| | ACCCAGCCCGAGCUGGUGCCCGAGGACCCCGAGGACUCCGCCCUGCUG | |
| | GAGGAUCCGGCCGGCACCGUAAGCUCCCAGAUCCCACCCAACUGGCAC | |
| | AUCCCCAGCAUCCAGGACGUGGCCCCACAUCACGCGCCUGCCGCUCCA | |
| | AGCAACCCCGGCCUGAUCAUUGGAGCUCUGGCCGGGAGCACUCUGGCG | |
| | GUGCUGGUGAUCGGCGGCAUCGCCUUCUGGGUGCGUCGGAGAGCCCAG | |
| | AUGGCCCCUAAGCGGCUGCGGCUGCCACACAUACGGGACGACGACGCG | |
| | CCACCAUCCCACCAGCCCCUGUUCUAC | |
| 3′ UTR | UAAAGCUCCCCGGGGGCCUCGGUGGCCUAGCUUCUUGCCCCUUGGGCC | SEQ ID |
| | UCCCCCCAGCCCCUCCUCCCCUUCCUGCACCCGUACCCCCGUGGUCUU | NO: 18 |
| | UGAAUAAAGUCUGAGUGGGCGGC | |
| Corresponding amino acid | MGRLTSGVGTAALLVVAVGLRVVCAKYALADPSLKMADPNRFRGKNLP | SEQ ID |
| sequence | VLDQLTDPPGVKRVYHIQPSLEDPFQPPSIPITVYYAVLERACRSVLL | NO: 19 |
| | HAPSEAPQIVRGASDEARKHTYNLTIAWYRMGDNCAIPITVMEYTECP | |
| | YNKSLGVCPIRTQPRWSYYDSFSAVSEDNLGFLMHAPAFETAGTYLRL | |
| | VKINDWTEITQFILEHRARASCKYALPLRIPPAACLTSKAYQQGVTVD | |
| | SIGMLPRFIPENQRTVALYSLKIAGWHGPKPPYTSTLLPPELSDTTNA | |
| | TQPELVPEDPEDSALLEDPAGTVSSQIPPNWHIPSIQDVAPHHAPAAP | |
| | SNPGLIIGALAGSTLAVLVIGGIAFWVRRRAQMAPKRLRLPHIRDDDA | |
| | PPSHQPLFY | |
| PolyA tail | 100 nt | |

COV2-2072_hc (Example 18)

| SEQ ID NO: 20 consists of from 5′ end to 3′ end: 5′ UTR SEQ ID NO: 16, mRNA | | SEQ ID |
|---|---|---|
| ORF SEQ ID NO: 21, and 3′ UTR SEQ ID NO: 4. | | NO: 20 |
| Chemistry | 1-methylpseudouridine | |
| Cap | 7mG(5′)ppp(5′)NlmpNp | |
| 5′ UTR | GGGAAAUCGCAAAAUUUGCUCUUCGCGUUAGAUUUCUUUUAGUUUUCU | SEQ ID |
| | CGCAACUAGCAAGCUUUUUGUUCUCGCC | NO: 16 |
| ORF of mRNA | AUGGAAACCGACACACUGCUGCUGUGGGUGCUGCUUCUUUGGGUGCCC | SEQ ID |
| (excluding the stop | GGAUCUACAGGAGAGGUGCAGCUGGUGCAGAGCGGCCCCGAGGUGAAG | NO: 21 |
| codon) | AAGCCCGGCACCAGCGUGAAGGUGAGCUGCAAGACCAGCGGCUUCACC | |
| | UUCACCAGCAGCGCCAUCCAGUGGGUGAGGCAGGCUAGAGGCCAGCGG | |
| | CUGGAGUGGAUCGGCUGGAUCGUGGUGGGCAGCGGCAACACCAACUAC | |
| | GCCCAGAAGUUCCAGGAGCGGGUGACCAUCACCCGGGACAUGAGCACC | |
| | AGCACCGCCUACAUGGAGCUGAGCAGCCUGCGGAGCGAGGACACCGCC | |
| | GUGUACUACUGCGCCGCCCCACACUGCAACCGGACCAGCUGCUACGAC | |
| | GCCUUCGACCUGUGGGGCCAGGGCACCAUGGUCACCGUGAGCAGCGCC | |
| | UCUACAAAGGGACCCAGCGUGUUCCCUCUGGCUCCUAGCAGCAAGAGC | |
| | ACAAGCGGAGGAACAGCCGCUCUGGGCUGUCUGGUCAAGGACUACUUU | |
| | CCCGAGCCUGUGACCGUGUCCUGGAAUUCUGGCGCUCUGACAUCCGGC | |
| | GUGCACACCUUUCCAGCUGUGCUGCAAAGCAGCGGCCUGUACUCUCUG | |
| | AGCAGCGUCGUGACAGUGCCAAGCAGCUCUCUGGGCACCCAGACCUAC | |
| | AUCUGCAACGUGAACCACAAGCCUAGCAACACCAAGGUGGACAAGAAG | |
| | GUGGAACCCAAGAGCUGCGACAAGACCCACACCUGUCCACCCUGUCCU | |
| | GCUCCAGAACUGCUCGGCGGACCUUCCGUGUUCCUGUUUCCUCCAAAG | |
| | CCUAAGGACACCCUGAUGAUCAGCAGAACACCCGAAGUGACCUGCGUG | |
| | GUGGUGGACGUGUCUCACGAGGACCCUGAAGUGAAGUUCAAUUGGUAC | |
| | GUGGACGGCGUGGAAGUGCACAACGCCAAGACCAAGCCUAGAGAGGAA | |
| | CAGUACAACAGCACCUACAGAGUGGUGUCCGUGCUGACCGUGCUGCAC | |
| | CAGGAUUGGCUGAACGGCAAAGAGUACAAGUGCAAGGUGUCCAACAAG | |
| | GCCCUGCCUGCUCCUAUCGAGAAGACCAUCAGCAAGGCCAAGGGCCAG | |
| | CCUAGGGAACCUCAGGUGUACACACUGCCUCCAAGCAGGGACGAGCUG | |
| | ACCAAGAAUCAGGUGUCCCUGACCUGCCUCGUGAAGGGCUUCUACCCU | |
| | UCCGAUAUCGCCGUGGAGUGGGAGAGCAACGGCCAGCCUGAGAACAAC | |
| | UACAAGACCACUCCUCCUGUGCUGGACAGCGACGGCUCAUUCUUCCUG | |
| | UACAGCAAGCUGACAGUGGACAAGUCCAGGUGGCAGCAGGGCAACGUG | |
| | UUCAGCUGCAGCGUGCUGCACGAAGCCCUGCACAGCCACUACACCCAG | |
| | AAGUCCCUGUCUCUGAGCCCUGGCAAA | |
| 3′ UTR | UGAUAAUAGGCUGGAGCCUCGGUGGCCUAGCUUCUUGCCCCUUGGGCC | SEQ ID |
| | UCCCCCCAGCCCCUCCUCCCCUUCCUGCACCCGUACCCCCGUGGUCUU | NO: 4 |
| | UGAAUAAAGUCUGAGUGGGCGGC | |
| Corresponding amino acid | METDTLLLWVLLLWVPGSTGEVQLVQSGPEVKKPGTSVKVSCKTSGFT | SEQ ID |
| sequence | FTSSAIQWVRQARGQRLEWIGWIVVGSGNTNYAQKFQERVTITRDMST | NO: 22 |
| | STAYMELSSLRSEDTAVYYCAAPHCNRTSCYDAFDLWGQGTMVTVSSA | |
| | STKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDEL | |
| | TKNQVSLTCLVKGFYPSDIAVEWESNGQPENNYKTTPPVLDSDGSFFL | |
| | YSKLTVDKSRWQQGNVFSCSVLHEALHSHYTQKSLSLSPGK | |
| PolyA tail | 100 nt | |

COV2-2072_lc_kappa (Example 18)

| SEQ ID NO: 23 consists of from 5′ end to 3′ end: 5′ UTR SEQ ID NO: 16, mRNA | | SEQ ID |
|---|---|---|
| ORF SEQ ID NO: 24, and 3′ UTR SEQ ID NO: 4. | | NO: 23 |
| Chemistry | 1-methylpseudouridine | |
| Cap | 7mG(5′)ppp(5′)NlmpNp | |
| 5′ UTR | GGGAAAUCGCAAAAUUUGCUCUUCGCGUUAGAUUUCUUUUAGUUUUCU | SEQ ID |
| | CGCAACUAGCAAGCUUUUUGUUCUCGCC | NO: 16 |
| ORF of mRNA | AUGGAAACCGACACACUGCUGCUGUGGGUGCUGCUUCUUUGGGUGCCC | SEQ ID |
| (excluding the stop | GGAUCUACAGGAGAGGUGCAGCUGGUGCAGAGCGGCCCCGAGGUGAAG | NO: 24 |
| codon) | AAGCCCGGCACCAGCGUGAAGGUGAGCUGCAAGACCAGCGGCUUCACC | |
| | UUCACCAGCAGCGCCAUCCAGUGGGUGAGGCAGGCUAGAGGCCAGCGG | |
| | CUGGAGUGGAUCGGCUGGAUCGUGGUGGGCAGCGGCAACACCAACUAC | |
| | GCCCAGAAGUUCCAGGAGCGGGUGACCAUCACCCGGGACAUGAGCACC | |
| | AGCACCGCCUACAUGGAGCUGAGCAGCCUGCGGAGCGAGGACACCGCC | |
| | GUGUACUACUGCGCCGCCCCACACUGCAACCGGACCAGCUGCUACGAC | |
| | GCCUUCGACCUGUGGGGCCAGGGCACCAUGGUCACCGUGAGCAGCGCC | |
| | UCUACAAAGGGACCCAGCGUGUUCCCUCUGGCUCCUAGCAGCAAGAGC | |
| | ACAAGCGGAGGAACAGCCGCUCUGGGCUGUCUGGUCAAGGACUACUUU | |
| | CCCGAGCCUGUGACCGUGUCCUGGAAUUCUGGCGCUCUGACAUCCGGC | |
| | GUGCACACCUUUCCAGCUGUGCUGCAAAGCAGCGGCCUGUACUCUCUG | |
| | AGCAGCGUCGUGACAGUGCCAAGCAGCUCUCUGGGCACCCAGACCUAC | |
| | AUCUGCAACGUGAACCACAAGCCUAGCAACACCAAGGUGGACAAGAAG | |
| | GUGGAACCCAAGAGCUGCGACAAGACCCACACCUGUCCACCCUGUCCU | |
| | GCUCCAGAACUGCUCGGCGGACCUUCCGUGUUCCUGUUUCCUCCAAAG | |
| | CCUAAGGACACCCUGAUGAUCAGCAGAACACCCGAAGUGACCUGCGUG | |
| | GUGGUGGACGUGUCUCACGAGGACCCUGAAGUGAAGUUCAAUUGGUAC | |
| | GUGGACGGCGUGGAAGUGCACAACGCCAAGACCAAGCCUAGAGAGGAA | |
| | CAGUACAACAGCACCUACAGAGUGGUGUCCGUGCUGACCGUGCUGCAC | |
| | CAGGAUUGGCUGAACGGCAAAGAGUACAAGUGCAAGGUGUCCAACAAG | |
| | GCCCUGCCUGCUCCUAUCGAGAAGACCAUCAGCAAGGCCAAGGGCCAG | |
| | CCUAGGGAACCUCAGGUGUACACACUGCCUCCAAGCAGGGACGAGCUG | |
| | ACCAAGAAUCAGGUGUCCCUGACCUGCCUCGUGAAGGGCUUCUACCCU | |
| | UCCGAUAUCGCCGUGGAGUGGGAGAGCAACGGCCAGCCUGAGAACAAC | |
| | UACAAGACCACUCCUCCUGUGCUGGACAGCGACGGCUCAUUCUUCCUG | |
| | UACAGCAAGCUGACAGUGGACAAGUCCAGGUGGCAGCAGGGCAACGUG | |
| | UUCAGCUGCAGCGUGCUGCACGAAGCCCUGCACAGCCACUACACCCAG | |
| | AAGUCCCUGUCUCUGAGCCCUGGCAAA | |
| 3′ UTR | UGAUAAUAGGCUGGAGCCUCGGUGGCCUAGCUUCUUGCCCCUUGGGCC | SEQ ID |
| | UCCCCCCAGCCCCUCCUCCCCUUCCUGCACCCGUACCCCCGUGGUCUU | NO: 4 |
| | UGAAUAAAGUCUGAGUGGGCGGC | |
| Corresponding amino acid | METPAQLLFLLLLWLPDTTGEIVLTQSPGTLSLSPGERATLSCRASQS | SEQ ID |
| sequence | VSSSYLGWYQQKPGQAPRLLIYGASSRATGIPDRFSGSGSGTDFTLTI | NO: 25 |
| | SRLEPEDFAVYYCQQYGSSPWTFGQGTKVEIKRTVAAPSVFIFPPSDE | |
| | QLKSGTASVVCLLNNFYPREAKVQWKVDNALQSGNSQESVTEQDSKDS | |
| | TYSLSSTLTLSKADYEKHKVYACEVTHQGLSSPVTKSFNRGEC | |
| PolyA tail | 100 nt | |

H1/N1 A/Victoria/2019 HA (Example 20)

| SEQ ID NO: 26 consists of from 5′ end to 3′ end: 5′ UTR SEQ ID NO: 2, mRNA | | SEQ ID |
|---|---|---|
| ORF SEQ ID NO: 27, and 3′ UTR SEQ ID NO: 4. | | NO: 26 |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCGGC | SEQ ID |
| | GCCGCCACC | NO: 2 |
| ORF of mRNA | AUGAAGGCCAUCCUGGUCGUGAUGCUGUACACCUUCACCACCGCCAAC | SEQ ID |
| (excluding the stop codon) | GCCGACACCCUGUGCAUCGGCUACCACGCCAACAACAGCACCGACACC | NO: 27 |
| | GUGGACACCGUGCUGGAGAAGAACGUGACCGUGACCCACAGCGUGAAC | |
| | CUGCUGGAGGACAAGCACAACGGCAAGCUGUGCAAGCUGAGGGGAGUG | |
| | GCACCCCUGCACCUGGGCAAGUGCAACAUCGCCGGCUGGAUCCUGGGC | |
| | AACCCCGAGUGCGAGAGCCUGAGCACAGCCCGGAGCUGGAGCUACAUC | |
| | GUGGAGACCAGCAACAGCGACAACGGCACCUGUUACCCCGGCGACUUC | |
| | AUCAACUACGAGGAGCUGCGGGAGCAGCUGAGCAGCGUGAGCAGCUUC | |
| | GAGCGGUUCGAGAUCUUCCCCAAGACCAGCAGCUGGCCCAACCACGAC | |
| | AGCGACAACGGCGUGACAGCAGCCUGUCCACACGCCGGAGCCAAGAGC | |
| | UUCUACAAGAACCUGAUCUGGCUGGUGAAGAAGGGCAAGAGCUACCCC | |
| | AAGAUCAACCAGACCUACAUCAACGACAAGGGCAAGGAGGUGCUGGUG | |
| | CUGUGGGGCAUCCACCACCCACCUACCAUCGCCGACCAGCAGAGCCUG | |
| | UACCAGAACGAGGACGCCUACGUGUUCGUGGGCACCAGCCGGUACAGC | |
| | AAGAAGUUCAAGCCAGAGAUCGCCACCCGGCCCAAGGUGAGAGACAGA | |
| | GAGGGCCGGAUGAACUACUACUGGACCCUGGUGGAGCCCGGAGACAAG | |
| | AUUACCUUCGAGGCCACCGGCAACCUGGUGGCCCCUCGGUACGCCUUC | |
| | ACCAUGGAACGGGACGCUGGCAGCGGCAUCAUCAUCAGCGACACUCCC | |
| | GUGCACGACUGCAACACCACCUGCCAGACUCCCGAGGGCGCUAUCAAC | |
| | ACCAGCCUGCCCUUCCAGAACGUGCACCCCAUCACCAUCGGCAAGUGC | |
| | CCCAAGUACGUAAAGAGCACCAAAUUGCGGCUGGCCACCGGACUCAGG | |
| | AACGUGCCCAGCAUCCAAAGCCGGGGCCUGUUUGGCGCAAUCGCCGGC | |
| | UUCAUCGAGGGCGGCUGGACUGGCAUGGUGGACGGCUGGUACGGCUAC | |
| | CACCACCAGAACGAACAGGGGAGCGGCUACGCAGCUGACCUGAAGAGC | |
| | ACCCAGAACGCCAUCGACAAGAUCACCAACAAGGUGAACAGCGUGAUC | |
| | GAGAAGAUGAACACCCAGUUCACCGCCGUGGGCAAGGAGUUCAACCAC | |
| | CUGGAGAAGCGGAUCGAGAACCUGAACAAGAAGGUGGACGACGGCUUC | |
| | CUGGACAUCUGGACCUACAACGCCGAGCUGCUGGUUCUGCUGGAGAAC | |
| | GAGCGGACCCUGGACUAUCACGACAGCAACGUGAAGAACCUGUACGAG | |
| | AAGGUGCGGAACCAGCUGAAGAACAACGCCAAGGAGAUCGGCAACGGC | |
| | UGCUUCGAGUUCUACCACAAGUGCGACAACACCUGCAUGGAGAGCGUG | |
| | AAGAACGGCACCUACGACUACCCCAAGUACAGCGAGGAGGCCAAGCUG | |
| | AACCGGGAGAAGAUCGACGGCGUGAAGCUGGACAGCACCCGGAUCUAC | |
| | CAGAUCCUGGCCAUCUACAGCACCGUGGCCAGCAGCCUGGUGCUGGUG | |
| | GUGAGCCUGGGCGCCAUCAGCUUCUGGAUGUGCAGCAACGGCAGCCUG | |
| | CAGUGCCGGAUCUGCAUC | |
| 3′ UTR | UGAUAAUAGGCUGGAGCCUCGGUGGCCUAGCUUCUUGCCCCUUGGGCC | SEQ ID |
| | UCCCCCCAGCCCCUCCUCCCCUUCCUGCACCCGUACCCCCGUGGUCUU | NO: 4 |
| | UGAAUAAAGUCUGAGUGGGCGGC | |
| Corresponding amino acid | MKAILVVMLYTFTTANADTLCIGYHANNSTDTVDTVLEKNVTVTHSVN | SEQ ID |
| sequence | LLEDKHNGKLCKLRGVAPLHLGKCNIAGWILGNPECESLSTARSWSYI | NO: 28 |
| | VETSNSDNGTCYPGDFINYEELREQLSSVSSFERFEIFPKTSSWPNHD | |
| | SDNGVTAACPHAGAKSFYKNLIWLVKKGKSYPKINQTYINDKGKEVLV | |
| | LWGIHHPPTIADQQSLYQNEDAYVFVGTSRYSKKFKPEIATRPKVRDR | |
| | EGRMNYYWTLVEPGDKITFEATGNLVAPRYAFTMERDAGSGIIISDTP | |
| | VHDCNTTCQTPEGAINTSLPFQNVHPITIGKCPKYVKSTKLRLATGLR | |
| | NVPSIQSRGLFGAIAGFIEGGWTGMVDGWYGYHHQNEQGSGYAADLKS | |
| | TQNAIDKITNKVNSVIEKMNTQFTAVGKEFNHLEKRIENLNKKVDDGF | |
| | LDIWTYNAELLVLLENERTLDYHDSNVKNLYEKVRNQLKNNAKEIGNG | |
| | CFEFYHKCDNTCMESVKNGTYDYPKYSEEAKLNREKIDGVKLDSTRIY | |
| | QILAIYSTVASSLVLVVSLGAISFWMCSNGSLQCRICI | |
| PolyA tail | 100 nt | |

*Any one of the open reading frames and/or corresponding amino acid sequences described in the Table may include or exclude the signal sequence. It should also be understood that the signal sequence may be replaced by a different signal sequence.

All publications, patent applications, patents, and other references mentioned herein are incorporated by reference in their entirety. In case of conflict, the present specification, including definitions, will control.

1. A method for inducing a mucosal immune response, comprising
administering to a mucosal surface of a subject a composition comprising an mRNA encoding an antigen and a nanoparticle, wherein the nanoparticle comprises a lipid nanoparticle core comprising an ionizable lipid, a phospholipid, a structural lipid, and a PEG-lipid, and a cationic agent dispersed primarily on the outer surface of the core in an effective amount to induce a mucosal immune response.

2. The method of claim 1, wherein the mRNA is encapsulated within the core.

3. The method of claim 1, wherein the nanoparticle has a greater than neutral zeta potential at physiological pH.

4. The method of claim 1, wherein a weight ratio of the cationic agent to mRNA is about 1:1 to about 4:1, about 1.25:1 to about 3.75:1, about 1.25:1, about 2.5:1, or about 3.75:1.

5. The method of claim 1, wherein the antigen is an infectious disease antigen.

6. The method of claim 1, wherein the mucosal surface comprises a cell population selected from respiratory mucosal cells, oral mucosal cells, intestinal mucosal cells, vaginal mucosal cells, rectal mucosal cells, and buccal mucosal cells.

7. A method for expressing a protein in mucosal tissue, comprising
administering to a mucosal surface of a subject a composition comprising an mRNA encoding a protein and a nanoparticle, wherein the nanoparticle comprises a lipid nanoparticle core comprising an ionizable lipid, a phospholipid, a structural lipid, and a PEG-lipid, and a cationic agent dispersed primarily on the outer surface of the core in an effective amount to induce expression of the protein in a mucosal tissue.

8. The method of claim 7, wherein the mRNA encodes a therapeutic protein.

9.-20. (canceled)

21. The method of claim 1, wherein greater than about 80%, greater than 90%, greater than 95%, or greater than 95% of the cationic agent is on the surface on the nanoparticle.

22. The method of claim 1, wherein at least about 50%, at least about 75%, at least about 90%, or at least about 95% of the mRNA is encapsulated within the core.

23-30. (canceled)

31. The method of claim 1, wherein the cationic agent is a cationic lipid and the cationic lipid is a sterol amine comprising a hydrophobic moiety and a hydrophilic moiety.

32. The method of claim 31, wherein the hydrophilic moiety comprises an amine group comprising one to four primary, secondary, or tertiary amines or mixtures thereof.

33.-35. (canceled)

36. The method of claim 32, wherein the amine group has a pKa value of greater than about 8.

37. (canceled)

38. The method of claim 31, wherein the sterol amine is a compound of Formula (A1):
A-L-B (A1)
or a salt thereof, wherein:
A is an amine group, L is an optional linker, and B is a sterol.

39. The method of claim 31, wherein the sterol amine has Formula A2a: 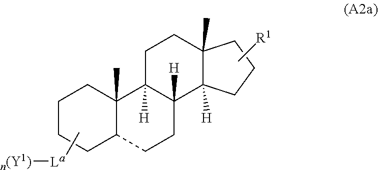
or a salt thereof, wherein: [symbol]
is a single or double bond;
R₁ is C₁₋₁₄ alkyl or C₁₋₁₄ alkenyl;
L^{a} is absent, —O—, —S—S—, —OC(═O)—, —C(═O)N—, —OC(═O)N—, CH₂—NH—C(O)—, —C(═O)O—, —OC(═O)—CH₂—CH₂—C(═O)N—, —S—S—CH₂, —SS—CH₂—CH₂—C(═O)N—, or a group of formula (a): 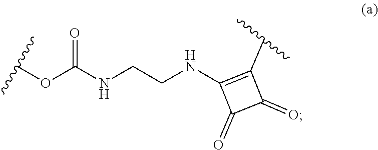 [SMILES: *OC(=O)NCCNc1c(*)c(=O)c1=O]
Y¹ is C₁₋₁₀ alkyl, 3 to 8-membered heterocycloalkyl, 5 to 6-membered heteroaryl, —C₁₋₆ alkyl-(3 to 8-membered heterocycloalkyl), or —C₁₋₆ alkyl-(5 to 6-membered heteroaryl),
wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) are each optionally substituted with 1, 2, 3, or 4 substituents independently selected from C₁₋₆ alkyl, halo, —OH, —O(C₁₋₆ alkyl), —C₁₋₆ alkyl-OH, —NH₂, —NH(C₁₋₆ alkyl), —N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH-(3 to 8-membered heterocycloalkyl), and —NH(5 to 6-membered heteroaryl); and
n is 1 or 2, and
optionally:
wherein [symbol] is a double bond,
wherein [symbol] is a single bond,
wherein L^{a} is —OC(═O)—, —OC(═O)N—, or —OC(═O)—CH₂—CH₂—C(═O)N—,
wherein n is 1,
wherein n is 2,
wherein R₁ is C₁₋₁₄ alkyl,
wherein R₁ is C₁₋₁₄ alkenyl,
wherein Rₜ is 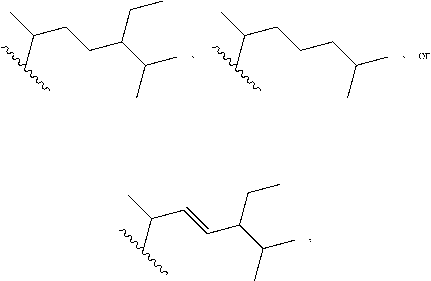
and/or
wherein Y^{t} is C₁₋₁₀ alkyl, 3 to 8-membered heterocycloalkyl, —C₁₋₆ alkyl-(3 to 8-membered heterocycloalkyl), or —C₁₋₆ alkyl-(5 to 6-membered heteroaryl), wherein the C₁₋₁₀ alkyl, 3 to 8-membered heterocycloalkyl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof; and wherein the C₁₋₁₀ alkyl, C₁₋₆ alkyl-(3 to 8-membered heterocycloalkyl), and C₁₋₆ alkyl-(5 to 6-membered heteroaryl) are each optionally substituted with C₁₋₆ alkyl, —OH, —C₁₋₆ alkyl-OH, or —NH₂.

40. The method of claim 39, wherein Y¹ is selected from:
(1); 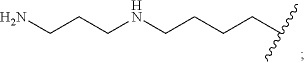 [SMILES: *CCCCNCCCN]
(2) 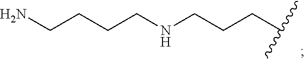 [SMILES: *CCCNCCCCN]
(3) 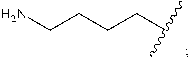 [SMILES: *CCCCN]
(4) 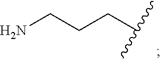 [SMILES: *CCCN]
(5) 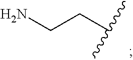 [SMILES: *CCN]
(6) 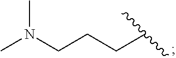 [SMILES: *CCCN(C)C]
(7) 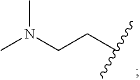 [SMILES: *CCN(C)C]
(8) 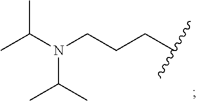 [SMILES: *CCCN(C(C)C)C(C)C]
(9) 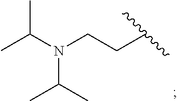 [SMILES: *CCN(C(C)C)C(C)C]
(10) 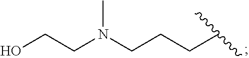 [SMILES: *CCCN(C)CCO]
(11) 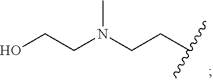 [SMILES: *CCN(C)CCO]
(12) 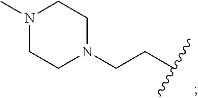 [SMILES: *CCN1CCN(C)CC1]
(13) 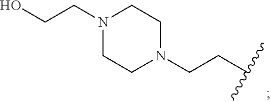 [SMILES: *CCN1CCN(CCO)CC1]
(14) 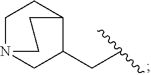 [SMILES: *CC1CN2CCC1CC2]
(15) 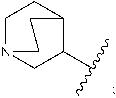 [SMILES: *C1CN2CCC1CC2]
(16) 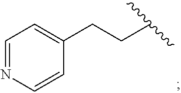 [SMILES: *CCc1ccncc1]
(17) 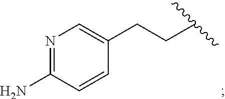 [SMILES: *CCc1ccc(N)nc1]
(18) 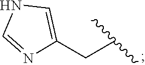 [SMILES: *Cc1c[nH]cn1]
(19) 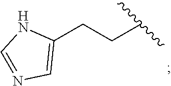 [SMILES: *CCc1cnc[nH]1]
(20) 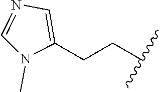 [SMILES: *CCc1cncn1C]
(21) 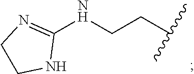 [SMILES: *CCNC1=NCCN1]
(22) 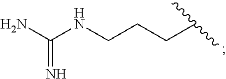 [SMILES: *CCCNC(=N)N]
(23) 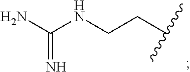 [SMILES: *CCNC(=N)N]
(28) —N(CH₃)₂; (29) 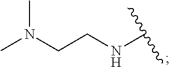 [SMILES: *NCCN(C)C]
(30) 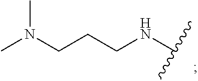 [SMILES: *NCCCN(C)C]
(31) 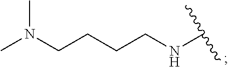 [SMILES: *NCCCCN(C)C]
and (32)

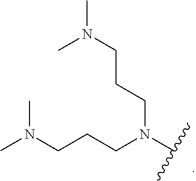 [SMILES: *N(CCCN(C)C)CCCN(C)C]

41. The method of claim 31, wherein the sterol amine has Formula A4 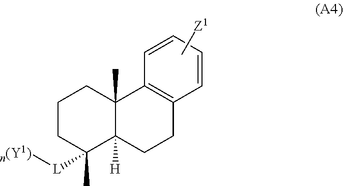
or a salt thereof, wherein:
Z¹ is —OH or C_{3.6} alkyl;
L is absent, —O—, —S—S—, —OC(═O)—, —C(═O)N—, —OC(═O)N—, —CH₂—NH—C(═O)—, —C(═O)O—, —OC(═O)—CH₂—CH₂—C(═O)N—, —S—S—CH₂—, or —SS—CH₂—CH₂—C(O)N—;
Y¹ is C₁₋₁₀ alkyl, 3 to 8-membered heterocycloalkyl, 5 to 6-membered heteroaryl, —C₁₋₆ alkyl-(3 to 8 membered heterocycloalkyl), or —C₁₋₆ alkyl-(5 to 6 membered heteroaryl),
wherein the C₁₋₁₀ alkyl, 3 to 8-membered heterocycloalkyl, 5 to 6-membered heteroaryl, —C₁₋₆ alkyl-(3 to 8-membered heterocycloalkyl), and —C₁₋₆ alkyl-(5 to 6-membered heteroaryl) comprises one to five primary, secondary, or tertiary amines or combination thereof;
and wherein the C₁₋₁₀ alkyl, 3 to 8-membered heterocycloalkyl, 5 to 6 membered heteroaryl, —C₁₋₆ alkyl-(3 to 8-membered heterocycloalkyl), and —C₁₋₆ alkyl-(5 to 6-membered heteroaryl) are each optionally substituted with 1, 2, 3, or 4 substituents selected from C₁₋₆ alkyl, halo, —OH, —O(C₁₋₆ alkyl), —C₁₋₆ alkyl-OH, —NH₂, —NH(C₁₋₆ alkyl), —N(C₁₋₆ alkyl)₂, 3 to 8-membered heterocycloalkyl (optionally substituted with C₁₋₁₄ alkyl comprising one to five primary, secondary, or tertiary amines or combination thereof), 5 to 6-membered heteroaryl, —NH(3 to 8-membered heterocycloalkyl), and —NH(5 to 6-membered heteroaryl); and
n is 1 or 2, and
optionally:
wherein Z¹ is —OH,
wherein Z¹ is C₃₋₆ alkyl,
wherein L is —C(═O)N—, —CH₂—NH—C(═O)—, or —C(═O)O—,
wherein Y¹ is C₁₋₁₀ alkyl comprising one to five primary, secondary, or tertiary amines or combination thereof,
wherein Y¹ is 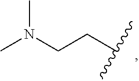
wherein n is 1, and/or
wherein n is 2.

42. The method of claim 31, wherein the sterol amine is selected from: SA3, SA10, SA18, SA24, SA58, SA78, SA121, SA137, SA138, SA158, and SA183.

43-46. (canceled)

47. The method of claim 1, wherein the ionizable lipid is a compound of Formula (I): 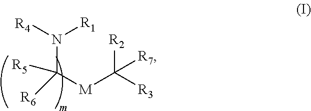
or a salt thereof, wherein:
R₁ is selected from the group consisting of C₅₋₃₀ alkyl, C₅₋₂₀ alkenyl, and —R″M′R′:
R₂ and R₃ are independently selected from the group consisting of C₁₋₁₄ alkyl and C₂₋₁₄ alkenyl;
R₄ is —(CH₂)ₙQ, wherein Q is —OR, wherein n is selected from 1, 2, 3, 4, and 5;
each R₅ is H;
each R₆ is H;
M and M′ are independently selected from —C(O)O— and —OC(O)—;
R₇ is H;
R is H;
R′ is selected from the group consisting of C₁₋₁₈ alkyl and C₂₋₁₈ alkenyl;
R″ is selected from the group consisting of C₃₋₁₄ alkyl and C₃₋₁₄ alkenyl; and
m is selected from 5, 6, 7, 8, 9, 10, 11, 12, and 13.

48. (canceled)

49. The method of claim 1, wherein the phospholipid is 1,2-distearoyl-sn-glycero-3-phosphocholine (DSPC).

50.-52. (canceled)


SEQUENCE LISTING (30 sequences)

SEQ ID NO: 1 - RNA, 3995 nt, Synthetic construct
  source 1..3995: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccaggacgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcacccggcagcggcggcagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagccccatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggacccgctctgcagatccccttccccatgcagatggcctaccggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 2 - RNA, 57 nt, Synthetic construct
  source 1..57: mol_type = other RNA
gggaaataagagagaaaagaagagtaagaagaaatataagaccccggcgccgccacc

SEQ ID NO: 3 - RNA, 3819 nt, Synthetic construct
  source 1..3819: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccaggacgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcacccggcagcggcggcagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagccccatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggacccgctctgcagatccccttccccatgcagatggcctaccggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 4 - RNA, 119 nt, Synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 5 - protein, 1273 aa, Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - RNA, 2571 nt, Synthetic construct
  source 1..2571: mol_type = other RNA
gaggaaataagagagaaaagaagagtaagaagaaatataagaccccggcgccgccaccatgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcacgtgaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaactgataataggctggagcctcggtggcctagcttcttgccccttgggcctccccccagcccctcctccccttcctgcacccgtacccccgtggtctttgaataaagtctgagtgggcggc

SEQ ID NO: 7 - RNA, 58 nt, Synthetic construct
  source 1..58: mol_type = other RNA
gaggaaataagagagaaaagaagagtaagaagaaatataagaccccggcgccgccacc

SEQ ID NO: 8 - RNA, 2394 nt, Synthetic construct
  source 1..2394: mol_type = other RNA
atgcggggtggcggactggtgtgcgccctggtggtcggtgcacttgtggccgctgtggctagcgctgcaccagccgcacccagagccagcggaggtgtggccgcgaccgttgcagccaacggaggcccggcttctcagcctcctcctgtgcccagccccgctaccaccaaggcccgtaagcggaagaccaagaagcctcccaagcggcccgaggctacaccaccacccgacgccaacgcaactgttgctgctggccacgccaccctgagggcccacctgcgggagatcaaggtggagaacgccgacgcccagttctacgtgtgtccacctccaaccggcgccaccgtggtacagttcgagcaacctcgccggtgccccactcgacccgagggccagaactacaccgagggcatcgccgtggtgttcaaggagaacatcgccccatacaagttcaaggccaccatgtactacaaggacgtgaccgtgagccaggtgtggttcggccaccggtacagccagttcatgggcatcttcgaggatcgggccccagtgcccttcgaggaggtgatcgacaagatcaacgccaagggcgtgtgccggagcaccgccaagtacgtgcggaacaacatggagaccaccgccttccaccgggacgaccacgagaccgacatggagctgaagcccgccaaggtggccacacggacaagccggggctggcacaccaccgacctgaagtacaacccaagccgcgtggaggcgttccaccggtacggcaccaccgtgaactgcatcgtggaggaggtggacgcccggagcgtgtacccctacgacgaattcgtgctggccaccggcgacttcgtgtacatgagccccttctacggctaccgggagggcagccacaccgagcacaccagctacgccgccgaccggttcaagcaggtggacggcttctacgcccgggacctgaccactaaagcccgggccacctcaccaaccacccggaacctgctgaccaccccaaagttcaccgtggcctgggactgggtgcccaagcgacccgccgtgtgcaccatgaccaagtggcaggaagtggacgagatgctgcgggccgaatacggcggcagcttccggttcagcagcgacgccatcagcaccaccttcaccaccaacctgacccagtacagcctgagccgggtggatctgggcgactgcatcggcagagacgctcgcgaggccatcgaccggatgttcgcccggaagtacaacgccacccacatcaaggtgggccagccccagtactacctcgccaccggcggcttcctgatcgcctaccagcccttgctgagcaacaccctggccgagctgtacgtgcgggagtacatgcgggagcaggaccggaagccccggaacgccacacccgcccctctgagagaagccccttccgccaacgccagcgtggagcggatcaagaccaccagcagcatcgagttcgcccggctgcagttcacctacaaccacatccagcggcacgtgaacgacatgctgggtcggatcgccgtggcttggtgcgagctgcagaaccacgagctgaccctgtggaacgaggcccggaagctgaaccccaacgccatcgccagcgctactgtgggccggagggtaagcgctcggatgctgggcgacgtgatggccgtgagcacgtgtgtgcccgtggccccagacaacgtgatcgtgcagaacagcatgcgggtgagtagccggcctggaacctgctacagccggcccctggtgtctttccggtacgaggaccagggtcccctgatcgagggccagctgggcgagaacaacgagctgcggctgactcgcgacgctctggagccctgcaccgtgggccacagacggtacttcatcttcggcggcggctacgtgtacttcgaggagtacgcctacagccaccagctgagccgggccgacgtgaccaccgtgagcaccttcatcgacctgaacatcaccatgctggaggaccacgagttcgtgcccctggaggtgtacacccggcacgagatcaaggacagcggcctgctggattacaccgaggtgcagcggcggaaccagctgcacgacctgcggttcgccgacatcgacaccgtgatacgggccgacgcaaacgccgccatgttcgccggcctgtgcgccttcttcgagggcatgggcgacctgggcagagccgtgggcaaggtggtgatgggcgtggtgggcggagtggtaagcgccgtgagcggcgtgagcagcttcatgagcaaccccttcggtgccctggcagtgggcctgctggtgcttgctggcctggtcgctgccttcttcgccttccggtacgtgctacagctgcagcggaac

SEQ ID NO: 9 - protein, 798 aa, Synthetic construct
MRGGGLVCALVVGALVAAVASAAPAAPRASGGVAATVAANGGPASQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRN

SEQ ID NO: 10 - RNA, 1607 nt, Synthetic construct
  source 1..1607: mol_type = other RNA
gaggaaatcgcaaaatttgctcttcgcgttagatttcttttagttttctcgcaactagcaagctttttgttctcgccatggcacttggacgcgtcggtctggctgtcggcctgtggggcctgctgtgggtgggcgtggtggtggtgctggctaacgccagccccggacgtaccatcaccgtggggcccagaggcaacgccagtaacgccgcgccatcagccagcccgagaaacgctagtgctccccggaccacgccaactccaccacagccccggaaggccaccaagagcaaggccagcaccgccaagcccgctccacctcccaagaccggcccacccaagaccagcagcgagcccgtgcggtgcaaccggcacgatcctctggcccggtacggctcacgggtgcagatccggtgccggttccccaacagcaccaggaccgagagccggctgcagatctggcggtacgccaccgccacagacgccgagatcggcaccgccccaagcctggaggaggtgatggtgaacgtgtctgctccacctggcggccagctggtgtacgacagcgcacccaaccggaccgatccccacgtgatctgggcagaaggcgctggtcctggcgctagccctcgactgtacagcgtggtcggcccactgggcaggcagcggctgatcatcgaggagctgaccctggagacccagggcatgtactactgggtgtggggccggacagatcggcctagcgcctacgggacctgggtgcgggttcgcgtgttccggccacctagcctgaccatccatccccacgccgtgctggagggccagcccttcaaagccacctgtaccgccgccacctactacccaggcaaccgggccgagttcgtgtggttcgaggacggacggcgcgtgttcgaccccgcccagatccacacccagacccaggagaaccccgacggcttcagcaccgtgagcacggtgaccagcgctgccgttggaggccaaggccctcccagaaccttcacctgccagctgacctggcaccgggacagcgtgagcgcttctcgccggaacgccagcggaactgccagcgtgctgccacggcccaccatcaccatggagttcaccggcgaccacgccgtgtgcacagccggctgcgtacccgagggcgtgaccttcgcctggttcctgggcgacgacagcagccccgccgagaaggtggccgtggccagccagaccagctgtggaagacccggaaccgccaccatccggagcaccctgcccgtgagctacgagcagaccgagtacatctgtcggctggccggctaccctgacggcatccccgtgttggagcaccacggcagccaccagcctcctcctcgggatcccaccgagcggcaggtgatcagggccgtggagggtgcaggcatcggcgtggccgtgctggtggccgtagtgctagccggcaccgccgtggtatacctgacctaaagctccccgggggcctcggtggcctagcttcttgccccttgggcctccccccagcccctcctccccttcctgcaggattgagactacgggtggtctttgaataaagtctgagtgggcggc

SEQ ID NO: 11 - RNA, 77 nt, Synthetic construct
  source 1..77: mol_type = other RNA
gaggaaatcgcaaaatttgctcttcgcgttagatttcttttagttttctcgcaactagcaagctttttgttctcgcc

SEQ ID NO: 12 - RNA, 1407 nt, Synthetic construct
  source 1..1407: mol_type = other RNA
atggcacttggacgcgtcggtctggctgtcggcctgtggggcctgctgtgggtgggcgtggtggtggtgctggctaacgccagccccggacgtaccatcaccgtggggcccagaggcaacgccagtaacgccgcgccatcagccagcccgagaaacgctagtgctccccggaccacgccaactccaccacagccccggaaggccaccaagagcaaggccagcaccgccaagcccgctccacctcccaagaccggcccacccaagaccagcagcgagcccgtgcggtgcaaccggcacgatcctctggcccggtacggctcacgggtgcagatccggtgccggttccccaacagcaccaggaccgagagccggctgcagatctggcggtacgccaccgccacagacgccgagatcggcaccgccccaagcctggaggaggtgatggtgaacgtgtctgctccacctggcggccagctggtgtacgacagcgcacccaaccggaccgatccccacgtgatctgggcagaaggcgctggtcctggcgctagccctcgactgtacagcgtggtcggcccactgggcaggcagcggctgatcatcgaggagctgaccctggagacccagggcatgtactactgggtgtggggccggacagatcggcctagcgcctacgggacctgggtgcgggttcgcgtgttccggccacctagcctgaccatccatccccacgccgtgctggagggccagcccttcaaagccacctgtaccgccgccacctactacccaggcaaccgggccgagttcgtgtggttcgaggacggacggcgcgtgttcgaccccgcccagatccacacccagacccaggagaaccccgacggcttcagcaccgtgagcacggtgaccagcgctgccgttggaggccaaggccctcccagaaccttcacctgccagctgacctggcaccgggacagcgtgagcgcttctcgccggaacgccagcggaactgccagcgtgctgccacggcccaccatcaccatggagttcaccggcgaccacgccgtgtgcacagccggctgcgtacccgagggcgtgaccttcgcctggttcctgggcgacgacagcagccccgccgagaaggtggccgtggccagccagaccagctgtggaagacccggaaccgccaccatccggagcaccctgcccgtgagctacgagcagaccgagtacatctgtcggctggccggctaccctgacggcatccccgtgttggagcaccacggcagccaccagcctcctcctcgggatcccaccgagcggcaggtgatcagggccgtggagggtgcaggcatcggcgtggccgtgctggtggccgtagtgctagccggcaccgccgtggtatacctgacc

SEQ ID NO: 13 - RNA, 123 nt, Synthetic construct
  source 1..123: mol_type = other RNA
taaagctccccgggggcctcggtggcctagcttcttgccccttgggcctccccccagcccctcctccccttcctgcaggattgagactacgggtggtctttgaataaagtctgagtgggcggc

SEQ ID NO: 14 - protein, 469 aa, Synthetic construct
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSASRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLT

SEQ ID NO: 15 - RNA, 1374 nt, Synthetic construct
  source 1..1374: mol_type = other RNA
gggaaatcgcaaaatttgctcttcgcgttagatttcttttagttttctcgcaactagcaagctttttgttctcgccatgggtcggctgaccagcggagttggcaccgccgcactcctggtggtggccgtaggcctgcgggtggtgtgcgccaagtacgccctggccgacccatccctgaagatggccgaccctaaccggttccggggcaagaatctgcccgtgctggatcagctgaccgatcctcctggcgtgaagcgggtgtaccacatccagcccagcctggaggatcccttccagccaccctccatccccatcaccgtttactacgccgtgctggagagagcctgccgcagcgtgctgctgcacgctccatccgaggcgccccagatcgtgcggggcgcaagcgacgaggcccggaagcacacctacaacctgaccatcgcctggtaccggatgggcgacaactgcgccatccctatcaccgtgatggagtacaccgagtgcccctacaacaagagcctgggagtgtgccccatccggacccagcctcggtggtcctactacgacagcttcagcgccgtgtccgaggacaacctgggcttcctgatgcacgcccctgccttcgagaccgccggcacctacctgcggctggtgaagatcaacgactggaccgagatcacccagttcatcctggagcaccgggcaagggccagctgcaagtacgcgctgcctctgcggatccctcccgctgcttgcctgaccagcaaggcctaccagcagggcgtgaccgtggacagcatcggcatgctgcccaggttcatccccgagaaccagcgcaccgtggccctgtacagcctgaagatcgcaggctggcacggacccaagcctccttacacctccaccctgctgccacccgagctgagcgacaccaccaacgccacccagcccgagctggtgcccgaggaccccgaggactccgccctgctggaggatccggccggcaccgtaagctcccagatcccacccaactggcacatccccagcatccaggacgtggccccacatcacgcgcctgccgctccaagcaaccccggcctgatcattggagctctggccgggagcactctggcggtgctggtgatcggcggcatcgccttctgggtgcgtcggagagcccagatggcccctaagcggctgcggctgccacacatacgggacgacgacgcgccaccatcccaccagcccctgttctactaaagctccccgggggcctcggtggcctagcttcttgccccttgggcctccccccagcccctcctccccttcctgcacccgtacccccgtggtctttgaataaagtctgagtgggcggc

SEQ ID NO: 16 - RNA, 76 nt, Synthetic construct
  source 1..76: mol_type = other RNA
gggaaatcgcaaaatttgctcttcgcgttagatttcttttagttttctcgcaactagcaagctttttgttctcgcc

SEQ ID NO: 17 - RNA, 1179 nt, Synthetic construct
  source 1..1179: mol_type = other RNA
atgggtcggctgaccagcggagttggcaccgccgcactcctggtggtggccgtaggcctgcgggtggtgtgcgccaagtacgccctggccgacccatccctgaagatggccgaccctaaccggttccggggcaagaatctgcccgtgctggatcagctgaccgatcctcctggcgtgaagcgggtgtaccacatccagcccagcctggaggatcccttccagccaccctccatccccatcaccgtttactacgccgtgctggagagagcctgccgcagcgtgctgctgcacgctccatccgaggcgccccagatcgtgcggggcgcaagcgacgaggcccggaagcacacctacaacctgaccatcgcctggtaccggatgggcgacaactgcgccatccctatcaccgtgatggagtacaccgagtgcccctacaacaagagcctgggagtgtgccccatccggacccagcctcggtggtcctactacgacagcttcagcgccgtgtccgaggacaacctgggcttcctgatgcacgcccctgccttcgagaccgccggcacctacctgcggctggtgaagatcaacgactggaccgagatcacccagttcatcctggagcaccgggcaagggccagctgcaagtacgcgctgcctctgcggatccctcccgctgcttgcctgaccagcaaggcctaccagcagggcgtgaccgtggacagcatcggcatgctgcccaggttcatccccgagaaccagcgcaccgtggccctgtacagcctgaagatcgcaggctggcacggacccaagcctccttacacctccaccctgctgccacccgagctgagcgacaccaccaacgccacccagcccgagctggtgcccgaggaccccgaggactccgccctgctggaggatccggccggcaccgtaagctcccagatcccacccaactggcacatccccagcatccaggacgtggccccacatcacgcgcctgccgctccaagcaaccccggcctgatcattggagctctggccgggagcactctggcggtgctggtgatcggcggcatcgccttctgggtgcgtcggagagcccagatggcccctaagcggctgcggctgccacacatacgggacgacgacgcgccaccatcccaccagcccctgttctac

SEQ ID NO: 18 - RNA, 119 nt, Synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 19 - protein, 393 aa, Synthetic construct
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 20 - RNA, 1614 nt, Synthetic construct
  source 1..1614: mol_type = other RNA
gggaaatcgcaaaatttgctcttcgcgttagatttcttttagttttctcgcaactagcaagctttttgttctcgccatggaaaccgacacactgctgctgtgggtgctgcttctttgggtgcccggatctacaggagaggtgcagctggtgcagagcggccccgaggtgaagaagcccggcaccagcgtgaaggtgagctgcaagaccagcggcttcaccttcaccagcagcgccatccagtgggtgaggcaggctagaggccagcggctggagtggatcggctggatcgtggtgggcagcggcaacaccaactacgcccagaagttccaggagcgggtgaccatcacccgggacatgagcaccagcaccgcctacatggagctgagcagcctgcggagcgaggacaccgccgtgtactactgcgccgccccacactgcaaccggaccagctgctacgacgccttcgacctgtggggccagggcaccatggtcaccgtgagcagcgcctctacaaagggacccagcgtgttccctctggctcctagcagcaagagcacaagcggaggaacagccgctctgggctgtctggtcaaggactactttcccgagcctgtgaccgtgtcctggaattctggcgctctgacatccggcgtgcacacctttccagctgtgctgcaaagcagcggcctgtactctctgagcagcgtcgtgacagtgccaagcagctctctgggcacccagacctacatctgcaacgtgaaccacaagcctagcaacaccaaggtggacaagaaggtggaacccaagagctgcgacaagacccacacctgtccaccctgtcctgctccagaactgctcggcggaccttccgtgttcctgtttcctccaaagcctaaggacaccctgatgatcagcagaacacccgaagtgacctgcgtggtggtggacgtgtctcacgaggaccctgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaagaccatcagcaaggccaagggccagcctagggaacctcaggtgtacacactgcctccaagcagggacgagctgaccaagaatcaggtgtccctgacctgcctcgtgaagggcttctacccttccgatatcgccgtggagtgggagagcaacggccagcctgagaacaactacaagaccactcctcctgtgctggacagcgacggctcattcttcctgtacagcaagctgacagtggacaagtccaggtggcagcagggcaacgtgttcagctgcagcgtgctgcacgaagccctgcacagccactacacccagaagtccctgtctctgagccctggcaaatgataataggctggagcctcggtggcctagcttcttgccccttgggcctccccccagcccctcctccccttcctgcacccgtacccccgtggtctttgaataaagtctgagtgggcggc

SEQ ID NO: 21 - RNA, 1419 nt, Synthetic construct
  source 1..1419: mol_type = other RNA
atggaaaccgacacactgctgctgtgggtgctgcttctttgggtgcccggatctacaggagaggtgcagctggtgcagagcggccccgaggtgaagaagcccggcaccagcgtgaaggtgagctgcaagaccagcggcttcaccttcaccagcagcgccatccagtgggtgaggcaggctagaggccagcggctggagtggatcggctggatcgtggtgggcagcggcaacaccaactacgcccagaagttccaggagcgggtgaccatcacccgggacatgagcaccagcaccgcctacatggagctgagcagcctgcggagcgaggacaccgccgtgtactactgcgccgccccacactgcaaccggaccagctgctacgacgccttcgacctgtggggccagggcaccatggtcaccgtgagcagcgcctctacaaagggacccagcgtgttccctctggctcctagcagcaagagcacaagcggaggaacagccgctctgggctgtctggtcaaggactactttcccgagcctgtgaccgtgtcctggaattctggcgctctgacatccggcgtgcacacctttccagctgtgctgcaaagcagcggcctgtactctctgagcagcgtcgtgacagtgccaagcagctctctgggcacccagacctacatctgcaacgtgaaccacaagcctagcaacaccaaggtggacaagaaggtggaacccaagagctgcgacaagacccacacctgtccaccctgtcctgctccagaactgctcggcggaccttccgtgttcctgtttcctccaaagcctaaggacaccctgatgatcagcagaacacccgaagtgacctgcgtggtggtggacgtgtctcacgaggaccctgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaagaccatcagcaaggccaagggccagcctagggaacctcaggtgtacacactgcctccaagcagggacgagctgaccaagaatcaggtgtccctgacctgcctcgtgaagggcttctacccttccgatatcgccgtggagtgggagagcaacggccagcctgagaacaactacaagaccactcctcctgtgctggacagcgacggctcattcttcctgtacagcaagctgacagtggacaagtccaggtggcagcagggcaacgtgttcagctgcagcgtgctgcacgaagccctgcacagccactacacccagaagtccctgtctctgagccctggcaaa

SEQ ID NO: 22 - protein, 473 aa, Synthetic construct
METDTLLLWVLLLWVPGSTGEVQLVQSGPEVKKPGTSVKVSCKTSGFTFTSSAIQWVRQARGQRLEWIGWIVVGSGNTNYAQKFQERVTITRDMSTSTAYMELSSLRSEDTAVYYCAAPHCNRTSCYDAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 23 - RNA, 1614 nt, Synthetic construct; identical to SEQ ID NO: 20
  source 1..1614: mol_type = other RNA

SEQ ID NO: 24 - RNA, 1419 nt, Synthetic construct; identical to SEQ ID NO: 21
  source 1..1419: mol_type = other RNA

SEQ ID NO: 25 - protein, 235 aa, Synthetic construct
METPAQLLFLLLLWLPDTTGEIVLTQSPGTLSLSPGERATLSCRASQSVSSSYLGWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - RNA, 1874 nt, Synthetic construct
  source 1..1874: mol_type = other RNA
gggaaataagagagaaaagaagagtaagaagaaatataagaccccggcgccgccaccatgaaggccatcctggtcgtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgaggggagtggcacccctgcacctgggcaagtgcaacatcgccggctggatcctgggcaaccccgagtgcgagagcctgagcacagcccggagctggagctacatcgtggagaccagcaacagcgacaacggcacctgttaccccggcgacttcatcaactacgaggagctgcgggagcagctgagcagcgtgagcagcttcgagcggttcgagatcttccccaagaccagcagctggcccaaccacgacagcgacaacggcgtgacagcagcctgtccacacgccggagccaagagcttctacaagaacctgatctggctggtgaagaagggcaagagctaccccaagatcaaccagacctacatcaacgacaagggcaaggaggtgctggtgctgtggggcatccaccacccacctaccatcgccgaccagcagagcctgtaccagaacgaggacgcctacgtgttcgtgggcaccagccggtacagcaagaagttcaagccagagatcgccacccggcccaaggtgagagacagagagggccggatgaactactactggaccctggtggagcccggagacaagattaccttcgaggccaccggcaacctggtggcccctcggtacgccttcaccatggaacgggacgctggcagcggcatcatcatcagcgacactcccgtgcacgactgcaacaccacctgccagactcccgagggcgctatcaacaccagcctgcccttccagaacgtgcaccccatcaccatcggcaagtgccccaagtacgtaaagagcaccaaattgcggctggccaccggactcaggaacgtgcccagcatccaaagccggggcctgtttggcgcaatcgccggcttcatcgagggcggctggactggcatggtggacggctggtacggctaccaccaccagaacgaacaggggagcggctacgcagctgacctgaagagcacccagaacgccatcgacaagatcaccaacaaggtgaacagcgtgatcgagaagatgaacacccagttcaccgccgtgggcaaggagttcaaccacctggagaagcggatcgagaacctgaacaagaaggtggacgacggcttcctggacatctggacctacaacgccgagctgctggttctgctggagaacgagcggaccctggactatcacgacagcaacgtgaagaacctgtacgagaaggtgcggaaccagctgaagaacaacgccaaggagatcggcaacggctgcttcgagttctaccacaagtgcgacaacacctgcatggagagcgtgaagaacggcacctacgactaccccaagtacagcgaggaggccaagctgaaccgggagaagatcgacggcgtgaagctggacagcacccggatctaccagatcctggccatctacagcaccgtggccagcagcctggtgctggtggtgagcctgggcgccatcagcttctggatgtgcagcaacggcagcctgcagtgccggatctgcatctgataataggctggagcctcggtggcctagcttcttgccccttgggcctccccccagcccctcctccccttcctgcacccgtacccccgtggtctttgaataaagtctgagtgggcggc

SEQ ID NO: 27 - RNA, 1698 nt, Synthetic construct
  source 1..1698: mol_type = other RNA
atgaaggccatcctggtcgtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgaggggagtggcacccctgcacctgggcaagtgcaacatcgccggctggatcctgggcaaccccgagtgcgagagcctgagcacagcccggagctggagctacatcgtggagaccagcaacagcgacaacggcacctgttaccccggcgacttcatcaactacgaggagctgcgggagcagctgagcagcgtgagcagcttcgagcggttcgagatcttccccaagaccagcagctggcccaaccacgacagcgacaacggcgtgacagcagcctgtccacacgccggagccaagagcttctacaagaacctgatctggctggtgaagaagggcaagagctaccccaagatcaaccagacctacatcaacgacaagggcaaggaggtgctggtgctgtggggcatccaccacccacctaccatcgccgaccagcagagcctgtaccagaacgaggacgcctacgtgttcgtgggcaccagccggtacagcaagaagttcaagccagagatcgccacccggcccaaggtgagagacagagagggccggatgaactactactggaccctggtggagcccggagacaagattaccttcgaggccaccggcaacctggtggcccctcggtacgccttcaccatggaacgggacgctggcagcggcatcatcatcagcgacactcccgtgcacgactgcaacaccacctgccagactcccgagggcgctatcaacaccagcctgcccttccagaacgtgcaccccatcaccatcggcaagtgccccaagtacgtaaagagcaccaaattgcggctggccaccggactcaggaacgtgcccagcatccaaagccggggcctgtttggcgcaatcgccggcttcatcgagggcggctggactggcatggtggacggctggtacggctaccaccaccagaacgaacaggggagcggctacgcagctgacctgaagagcacccagaacgccatcgacaagatcaccaacaaggtgaacagcgtgatcgagaagatgaacacccagttcaccgccgtgggcaaggagttcaaccacctggagaagcggatcgagaacctgaacaagaaggtggacgacggcttcctggacatctggacctacaacgccgagctgctggttctgctggagaacgagcggaccctggactatcacgacagcaacgtgaagaacctgtacgagaaggtgcggaaccagctgaagaacaacgccaaggagatcggcaacggctgcttcgagttctaccacaagtgcgacaacacctgcatggagagcgtgaagaacggcacctacgactaccccaagtacagcgaggaggccaagctgaaccgggagaagatcgacggcgtgaagctggacagcacccggatctaccagatcctggccatctacagcaccgtggccagcagcctggtgctggtggtgagcctgggcgccatcagcttctggatgtgcagcaacggcagcctgcagtgccggatctgcatc

SEQ ID NO: 28 - protein, 566 aa, Synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNEDAYVFVGTSRYSKKFKPEIATRPKVRDREGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 29 - RNA, 47 nt, Synthetic construct
  source 1..47: mol_type = other RNA
gggaaataagagagaaaagaagagtaagaagaaatataagagccacc

SEQ ID NO: 30 - RNA, 119 nt, Synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc